(19) United States
(12) Patent Application Publication
(10) Pub. No.: US 2023/0382925 A1
(43) Pub. Date: Nov. 30, 2023

(54) MODULATORS OF CYSTIC FIBROSIS TRANSMEMBRANE CONDUCTANCE REGULATOR

(71) Applicant: VERTEX PHARMACEUTICALS INCORPORATED, Boston, MA (US)

(72) Inventors: Jason MCCARTNEY, Cardiff by the Sea, CA (US); Alexander Russell ABELA, Escondido, CA (US); Sunny ABRAHAM, San Diego, CA (US); Corey Don ANDERSON, Brighton, MA (US); Vijayalaksmi ARUMUGAM, San Marcos, CA (US); Jaclyn CHAU, San Diego, CA (US); Jeremy CLEMENS, San Diego, CA (US); Thomas CLEVELAND, San Marcos, CA (US); Timothy Richard COON, Carlsbad, CA (US); Timothy A. DWIGHT, Quincy, MA (US); Lev Tyler Dewey FANNING, San Marcos, CA (US); Bryan A. FRIEMAN, La Jolla, CA (US); Peter GROOTENHUIS, Del Mar, CA (US); Anton V. GULEVICH, San Diego, CA (US); Sara Sabina HADIDA RUAH, La Jolla, CA (US); Yoshihiro ISHIHARA, San Diego, CA (US); Haripada KHATUYA, San Diego, CA (US); Paul KRENITSKY, San Francisco, CA (US); Vito MELILLO, Escondido, CA (US); Mark Thomas MILLER, Rancho Santa Fe, CA (US); Prasuna PARASELLI, San Diego, CA (US); Fabrice PIERRE, La Jolla, CA (US); Alina SILINA, Needham, MA (US); Joe A. TRAN, San Marcos, CA (US); Johnny UY, San Diego, CA (US); Lino VALDEZ, San Diego, CA (US); Jinglan ZHOU, San Diego, CA (US)

(21) Appl. No.: 18/030,529

(22) PCT Filed: Oct. 6, 2021
(86) PCT No.: PCT/US2021/053860

§ 371 Date: Apr. 6, 2023

Related U.S. Application Data
(60) Provisional application No. 63/088,935, filed on Oct. 7, 2020.

(51) Int. Cl. C07D 515/08; A61K 45/06

(52) U.S. Cl. CPC C07D 515/08; A61K 45/06

(57) ABSTRACT

This disclosure provides modulators of Cystic Fibrosis Transmembrane Conductance Regulator (CFTR) having the core structure: pharmaceutical compositions containing at least one such modulator, methods of treatment of CFTR mediated diseases, including cystic fibrosis, using such modulators and pharmaceutical compositions, combination pharmaceutical compositions and combination therapies, and processes and intermediates for making such modulators.

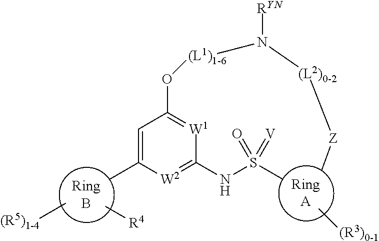

This application claims the benefit of priority of U.S. Provisional Application No. 63/088,935, filed Oct. 7, 2020, the contents of which are incorporated by reference herein in their entirety.

The disclosure relates to modulators of Cystic Fibrosis Transmembrane Conductance Regulator (CFTR), pharmaceutical compositions containing the modulators, methods of treating CFTR mediated diseases, including cystic fibrosis, using such modulators and pharmaceutical compositions, combination therapies and combination pharmaceutical compositions employing such modulators, and processes and intermediates for making such modulators.

Cystic fibrosis (CF) is a recessive genetic disease that affects approximately 70,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to increased mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, greater than 2000 mutations in the CF gene have been identified; currently, the CFTR2 database contains information on only 432 of these identified mutations, with sufficient evidence to define 352 mutations as disease causing. The most prevalent disease-causing mutation is a deletion of phenylalanine at position 508 of the CFTR amino acid sequence and is commonly referred to as the F508del mutation. This mutation occurs in many of the cases of cystic fibrosis and is associated with severe disease.

The deletion of residue 508 in CFTR prevents the nascent protein from folding correctly. This results in the inability of the mutant protein to exit the endoplasmic reticulum (ER) and traffic to the plasma membrane. As a result, the number of CFTR channels for anion transport present in the membrane is far less than observed in cells expressing wild-type CFTR, i.e., CFTR having no mutations. In addition to impaired trafficking, the mutation results in defective channel gating. Together, the reduced number of channels in the membrane and the defective gating lead to reduced anion and fluid transport across epithelia. (Quinton, P. M. (1990), FASEB J. 4: 2709-2727). The channels that are defective because of the F508del mutation are still functional, albeit less functional than wild-type CFTR channels. (Dalemans et al. (1991), Nature Lond. 354: 526-528; Pasyk and Foskett (1995), J. Cell. Biochem. 270: 12347-50). In addition to F508del, other disease-causing mutations in CFTR that result in defective trafficking, synthesis, and/or channel gating could be up- or down-regulated to alter anion secretion and modify disease progression and/or severity.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of 1480 amino acids that encode a protein which is made up of a tandem repeat of transmembrane domains, each containing six transmembrane helices and a nucleotide binding domain. The two transmembrane domains are linked by a large, polar, regulatory (R)—domain with multiple phosphorylation sites that regulate channel activity and cellular trafficking.

Chloride transport takes place by the coordinated activity of ENaC and CFTR present on the apical membrane and the Na⁺—K⁺-ATPase pump and Cl— channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Na⁺—K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

A number of CFTR modulating compounds have recently been identified. However, compounds that can treat or reduce the severity of cystic fibrosis and other CFTR mediated diseases, and particularly the more severe forms of these diseases, are still needed.

One aspect of the disclosure provides novel compounds, including compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing.

Formula I encompasses compounds falling within the following structure:

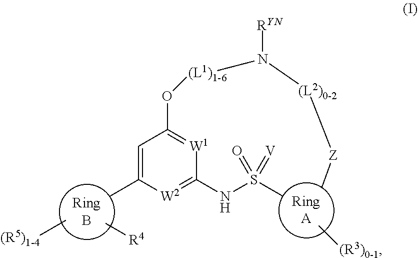

and includes tautomers of those compounds, deuterated derivatives of any of the compounds and tautomers, and pharmaceutically acceptable salts of any of the foregoing, wherein:
Ring A is selected from:
   C₆-C₁₀ aryl,
   C₃-C₁₀ cycloalkyl,
   3- to 10-membered heterocyclyl, and
   5- to 10-membered heteroaryl;
Ring B is selected from:
   C₆-C₁₀ aryl,
   C₃-C₁₀ cycloalkyl,
   3- to 10-membered heterocyclyl, and
   5- to 10-membered heteroaryl;
V is selected from O and NH;
W¹ is selected from N and CH;
W² is selected from N and CH, provided that at least one of W¹ and W² is N;
Z is selected from O, NR^{ZN}, and C(R^{ZC})₂, provided that when L² is absent, Z is C(R^{ZC})₂
each L¹ is independently selected from C(R^{L1})₂ and

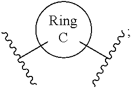

each L² is independently selected from C(R^{L2})₂;
Ring C is selected from C₆-C₁₀ aryl optionally substituted with 1-3 groups independently selected from:
   halogen,
   C₁-C₆ alkyl, and
   N(R^{N})₂;
each R³ is independently selected from:
   halogen,
   C₁-C₆ alkyl,
   C₁-C₆ alkoxy,
   C₃-C₁₀ cycloalkyl,
   C₆-C₁₀ aryl optionally substituted with 1-3 groups independently selected from C₁-C₆ alkyl, and
   3- to 10-membered heterocyclyl;
R⁴ is selected from hydrogen and C₁-C₆ alkyl;
each R⁵ is independently selected from:
   hydrogen,
   halogen,
   hydroxyl,
   N(R^{N})₂,
   —SO-Me,
   CH═C(R^{LC})₂, wherein both R^{LC} are taken together to form a C₃-C₁₀ cycloalkyl,
      C₁-C₆ alkyl optionally substituted with 1-3 groups independently selected from:
      hydroxyl,
      C₁-C₆ alkoxy optionally substituted with 1-3 groups independently selected from C₁-C₆ alkoxy and C₆-C₁₀ aryl,
      C₃-C₁₀ cycloalkyl,
      —(O)₀₋₁—(C₆-C₁₀ aryl) optionally substituted with 1-3 groups independently selected from C₁-C₆ alkyl and C₁-C₆ alkoxy,
      3- to 10-membered heterocyclyl, and
      N(R^{N})₂,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl,
   C₆-C₁₀ aryl, and
   3- to 10-membered heterocyclyl;
R^{YN} is selected from:
   C₃-C₁₀ cycloalkyl optionally substituted with 1-3 groups independently selected from:
      hydroxyl,
      oxo,
      halogen,
      cyano,
      N(R^{N})₂,
         C₁-C₆ alkyl optionally substituted with 1-3 groups independently selected from:
         hydroxyl,
         oxo,
         N(R^{N})₂,
         C₁-C₆ alkoxy, and
         C₆-C₁₀ aryl,
      C₁-C₆ alkoxy optionally substituted with 1-3 groups independently selected from halogen, oxo, C₆-C₁₀ aryl, and N(R^{N})₂,
      halogen,
      C₃-C₁₀ cycloalkyl,
      3- to 10-membered heterocyclyl optionally substituted with 1-3 groups independently selected from C₁-C₆ alkyl, and
      5- to 10-membered heteroaryl optionally substituted with 1-3 groups independently selected from:
         hydroxyl,
         cyano,
         oxo,
         halogen,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         C₆-C₁₀ aryl, and
         3- to 10-membered heterocyclyl optionally substituted with 1-3 groups independently selected from C₁-C₆ alkyl,
   C₆-C₁₀ aryl,
   3- to 10-membered heterocyclyl optionally substituted with 1-3 groups independently selected from:
      oxo,
      C₁-C₆ alkyl optionally substituted with 1-3 groups independently selected from:
         oxo,
         hydroxyl,
         N(R^{N})₂, and
         C₁-C₆ alkoxy optionally substituted with 1-3 groups independently selected from halogen and C₆-C₁₀ aryl, and-(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl, and
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl);
R^{ZN} is selected from:
   hydrogen,
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         hydroxyl,
         C₁-C₆ alkyl optionally substituted with 1-3 groups independently selected from hydroxyl, oxo, C₁-C₆ alkoxy, C₆-C₁₀ aryl, and N(R^{N})₂,
         C₁-C₆ fluoroalkyl,
         C₁-C₆ alkoxy,
         COOH,
         N(R^{N})₂,
         C₆-C₁₀ aryl, and
         3- to 10-membered heterocyclyl optionally substituted with 1-3 groups independently selected from oxo and C₁-C₆ alkyl,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl,
         —(O)₀₋₁—(C₆-C₁₀ aryl), and
         —(O)₀₋₁-(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
      3- to 10-membered heterocyclyl optionally substituted with 1-4 groups independently selected from:
         hydroxyl,
         oxo,
         N(R^{N})₂,
         C₁-C₆ alkyl optionally substituted with 1-3 groups independently selected from oxo and C₁-C₆ alkoxy,
         C₁-C₆ alkoxy,
         C₁-C₆ fluoroalkyl,
         C₆-C₁₀ aryl optionally substituted with 1-3 groups independently selected from halogen, and
         5- to 10-membered heteroaryl, and
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy (optionally substituted with 1-3 —SiMe₃), and N(R^{N})₂,
         C₁-C₆ alkoxy optionally substituted with 1-3 groups independently selected from hydroxyl, oxo, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         —(O)₀₋₁—(C₆-C₁₀ aryl),
         —(O)₀₋₁-(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, and 3- to 10-membered heterocyclyl (optionally substituted with 1-3 groups independently selected from C₁-C₆ fluoroalkyl), and
         5- to 10-membered heteroaryl optionally substituted with 1-4 groups independently selected from C₁-C₆ alkyl and C₃-C₁₀ cycloalkyl,
   C₁-C₆ fluoroalkyl,
   C₃-C₁₀ cycloalkyl optionally substituted with 1-3 groups independently selected from:
      hydroxyl,
      oxo,
      halogen,
      cyano,
      N(R^{N})₂,
      C₁-C₆ alkyl optionally substituted with 1-3 groups independently selected from:
         hydroxyl,
         oxo,
         N(R^{N})₂,
         C₁-C₆ alkoxy, and
         C₆-C₁₀ aryl,
      C₁-C₆ alkoxy optionally substituted with 1-3 groups independently selected from halogen, oxo, C₆-C₁₀ aryl, and N(R^{N})₂,
      halogen,
      C₃-C₁₀ cycloalkyl,
      3- to 10-memember heterocyclyl optionally substituted with 1-3 groups independently selected from C₁-C₆ alkyl, and
      5- to 10-membered heteroaryl optionally substituted with 1-3 groups independently selected from:
         hydroxyl,
         cyano,
         oxo,
         halogen,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         C₆-C₁₀ aryl, and
         3- to 10-membered heterocyclyl optionally substituted with 1-3 groups independently selected from C₁-C₆ alkyl,
   C₆-C₁₀ aryl,
   3- to 10-membered heterocyclyl optionally substituted with 1-3 groups independently selected from:
      oxo,
      C₁-C₆ alkyl optionally substituted with 1-3 groups independently selected from:
         oxo,
         hydroxyl,
         N(R^{N})₂,
         C₁-C₆ alkoxy optionally substituted with 1-3 groups independently selected from halogen and C₆-C₁₀ aryl, and
         —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl,
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
   R^{F};
each R^{ZC} is independently selected from:
   hydrogen,
   C₁-C₆ alkyl optionally substituted with 1-3 groups independently selected from C₆-C₁₀ aryl (optionally substituted with 1-3 groups independently selected from C₁-C₆ alkyl),
   C₆-C₁₀ aryl optionally substituted with 1-3 groups independently selected from C₁-C₆ alkyl, and
   R^{F};
or two R^{ZC} are taken together to form an oxo group;
each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen and C₁-C₆ fluoroalkyl,
      C₆-C₁₀ aryl optionally substituted with 1-3 groups independently selected from C₁-C₆ alkyl, and
      3- to 10-membered heterocyclyl optionally substituted with 1-3 groups independently selected from C₁-C₆ alkyl (optionally substituted with 1-3 groups independently selected from hydroxyl and oxo),
   C₃-C₁₀ cycloalkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from:
         hydroxyl,
         oxo,
         cyano,
         SiMe₃,
         N(R^{N})₂, and
         C₃-C₁₀ cycloalkyl optionally substituted with 1-3 groups independently selected from C₁-C₆ fluoroalkyl,
      C₁-C₆ alkoxy optionally substituted with 1-3 groups independently selected from:
         C₃-C₁₀ cycloalkyl optionally substituted with 1-3 groups independently selected from C₁-C₆ fluoroalkyl, and
         C₁-C₆ alkoxy,
      C₁-C₆ fluoroalkyl,
      C₃-C₁₀ cycloalkyl optionally substituted with 1-3 groups independently
      selected from C₁-C₆ alkyl and C₁-C₆ fluoroalkyl,
      C₆-C₁₀ aryl,
      3- to 10-membered heterocyclyl optionally substituted with 1-3 groups independently selected from C₁-C₆ alkyl, and
      5- to 10-membered heteroaryl,
   3- to 10-membered heterocyclyl optionally substituted with 1-3 groups independently selected from:
      C₁-C₆ alkyl optionally substituted with 1-3 groups independently selected from:
         oxo, and
         C₁-C₆ alkoxy,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
      C₆-C₁₀ aryl optionally substituted with 1-3 groups independently selected
   from C₁-C₆ alkyl, and
   R^{F};
or two R^{L1} on the same carbon atom are taken together to form an oxo group;
each R^{L2} is independently selected from hydrogen and R^{F};
or two R^{L2} on the same carbon atom are taken together to form an oxo group;
each R^{N} is independently selected from:
   hydrogen,
   C₁-C₈ alkyl optionally substituted with 1-3 groups independently selected from:
      oxo,
      halogen,
      hydroxyl,
      NH₂,
      NHMe,
      NMe₂,
   NHCOMe,
      C₁-C₆ alkoxy optionally substituted with 1-3 groups independently selected from C₆-C₁₀ aryl,
      —(O)₀₋₁—(C₃-C₁₀ cycloalkyl),
      C₆-C₁₀ aryl optionally substituted with 1-3 groups independently selected from halogen and C₁-C₆ alkyl,
      3- to 14-membered heterocyclyl optionally substituted with 1-4 groups independently selected from oxo and C₁-C₆ alkyl, and
      5- to 14-membered heteroaryl optionally substituted with 1-4 groups independently selected from oxo and C₁-C₆ alkyl,
   C₃-C₁₀ cycloalkyl optionally substituted with 1-3 groups independently selected from:
      hydroxyl,
      NH₂,
      NHMe, and
      C₁-C₆ alkyl optionally substituted with 1-3 groups independently selected from hydroxyl, and
   C₆-C₁₀ aryl, and
   3- to 10-membered heterocyclyl;
or two R^{N} on the same nitrogen atom are taken together with the nitrogen to which they are bonded to form a 3- to 10-membered heterocyclyl optionally substituted with 1-3 groups selected from:
   hydroxyl,
   oxo,
   cyano,
   C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
   C₁-C₆ alkoxy, and
   C₁-C₆ fluoroalkyl;
or one R⁴ and one R^{L1} are taken together to form a C₆-C₈ alkylene;
when R^{F} is present, two R^{F} taken together with the atoms to which they are bonded form a group selected from:
   C₃-C₁₀ cycloalkyl optionally substituted with 1-3 groups independently selected from C₁-C₆ alkyl,
   C₆-C₁₀ aryl optionally substituted with 1-3 groups independently selected from:
      halogen,
      C₁-C₆ alkyl,
      N(R^{N})₂, and
      3- to 10-membered heterocyclyl optionally substituted with 1-3 groups independently selected from hydroxyl,
   3- to 11-membered heterocyclyl optionally substituted with 1-3 groups independently selected from:
      oxo,
      N(R^{N})₂,
      C₁-C₉ alkyl optionally substituted with 1-4 groups independently selected from:
         oxo,
         halogen,
         hydroxyl,
         N(R^{N})₂,
         —SO₂—(C₁-C₆ alkyl),
         C₁-C₆ alkoxy optionally substituted with 1-3 groups independently selected from halogen, C₆-C₁₀ aryl,
         C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N},
         —O-(5- to 12-membered heteroaryl) optionally substituted with 1-3 groups independently selected from C₆-C₁₀ aryl (optionally substituted with 1-3 groups independently selected from halogen) and C₁-C₆ alkyl, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₃-C₁₂ cycloalkyl optionally substituted with 1-4 groups independently
      selected from halogen, C₁-C₆ alkyl, and C₁-C₆ fluoroalkyl,
      C₆-C₁₀ aryl,
      3- to 10-membered heterocyclyl, and
      5- to 10-membered heteroaryl optionally substituted with 1-3 groups independently selected from C₁-C₆ alkoxy and C₁-C₆ fluoroalkyl, and
5- to 12-membered heteroaryl optionally substituted with 1-3 groups independently selected from C₁-C₆ alkyl and C₁-C₆ fluoroalkyl;
   with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.

Formula I also includes compounds of Formula Ia:

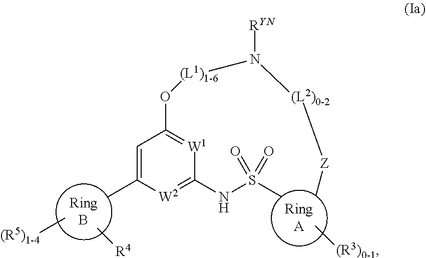

tautomers of those compounds, deuterated derivatives of any of the compounds and tautomers, and pharmaceutically acceptable salts of any of the foregoing, wherein Ring A, Ring B, W¹, W², Z, L¹, L², R³, R⁴, R⁵, and R^{YN} are as defined for Formula I, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.

Formula I also includes compounds of Formula IIa:

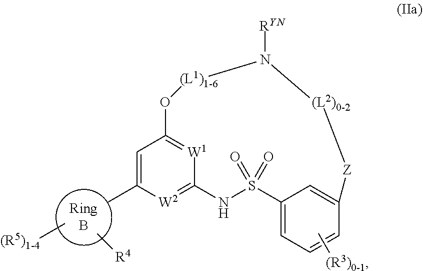

tautomers of those compounds, deuterated derivatives of any of the compounds and tautomers, and pharmaceutically acceptable salts of any of the foregoing, wherein Ring B, W¹, W², Z, L¹,
L², R³, R⁴, R⁵, and R^{YN} are as defined for Formula I, with the proviso that the compound is not selected from.
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.

Formula I also includes compounds of Formula IIb:

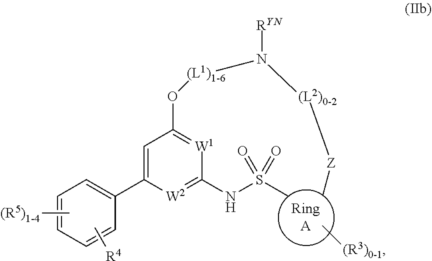

tautomers of those compounds, deuterated derivatives of any of the compound and tautomers, and pharmaceutically acceptable salts of any of the foregoing, wherein Ring A, W¹, W², Z, L¹, L², R³, R⁴, R⁵, and R^{YN} are as defined for Formula I, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.

Formula I also includes compounds of Formula III:

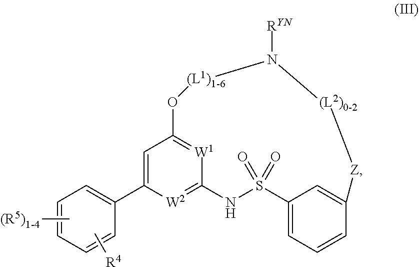

tautomers of those compounds, deuterated derivatives of any of the compounds and tautomers, and pharmaceutically acceptable salts of any of the foregoing, wherein W¹, W², Z, L¹, L², R⁴, R⁵, and R^{YN} are as defined for Formula I, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.

Formula I also includes compounds of Formula IV:

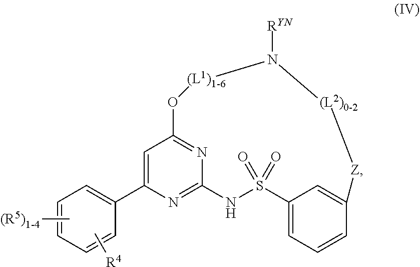

tautomers of those compounds, deuterated derivatives of any of the compounds and tautomers, and pharmaceutically acceptable salts of any of the foregoing, wherein Z, L¹, L², R¹, R⁴, R⁵, and R^{YN} are as defined for Formula I, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.

Formula I also includes compounds of Formula V:

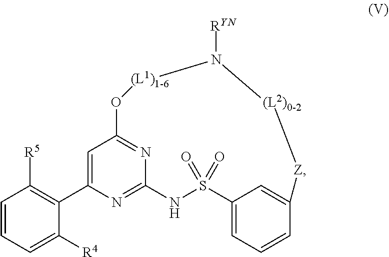

tautomers of those compounds, deuterated derivatives of any of the compounds and tautomers, and pharmaceutically acceptable salts of any of the foregoing, wherein Z, L¹, L², R⁴, R⁵, and R^{YN} are as defined for Formula I, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.

Formula I also includes compounds of Formula VI:

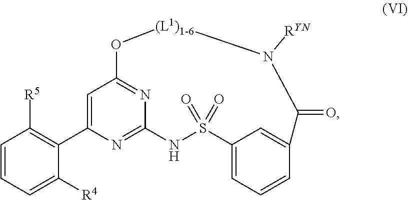

tautomers of those compounds, deuterated derivatives of any of the compounds and tautomers, and pharmaceutically acceptable salts of any of the foregoing, wherein L¹, R⁴, R⁵, and R^{YN} are as defined for Formula I, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.

Another aspect of the disclosure provides pharmaceutical compositions comprising at least one compound chosen from the novel compounds disclosed herein, tautomers thereof, deuterated derivatives of those compounds and tautomers, and pharmaceutically acceptable salts of any of the foregoing, and at least one pharmaceutically acceptable carrier. These compositions may further include at least one additional active pharmaceutical ingredient. In some embodiments of the pharmaceutical compositions disclosed herein, the at least one additional active pharmaceutical ingredient is at least one other CFTR modulator. In some embodiments, the at least one other CFTR modulator is selected from CFTR potentiators and CFTR modulators.

Thus, another aspect of the disclosure provides methods of treating the CFTR-mediated disease cystic fibrosis comprising administering at least one of compound chosen from the novel compounds disclosed herein, pharmaceutically acceptable salts thereof, and deuterated derivatives of any of the foregoing, and at least one pharmaceutically acceptable carrier. In some embodiments, the methods comprise administering a pharmaceutical composition disclosed herein, wherein the pharmaceutical composition comprises at least one additional active pharmaceutical ingredient. In some embodiments, the at least one additional active ingredient is at least one other CFTR modulator. In some embodiments, the at least one other CFTR modulator is selected from CFTR potentiators and CFTR modulators.

In certain embodiments, the pharmaceutical compositions of the disclosure comprise at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing. In some embodiments, compositions comprising at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, may optionally further comprise (a) at least one compound chosen from (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (tezacaftor), 3-(6-(1-(2,2-difluorobenzo[d][1,3]dioxol-5-yl)cyclopropane carboxamido)-3-methylpyridin-2-yl)benzoic acid (lumacaftor), and deuterated derivatives and pharmaceutically acceptable salts of tezacaftor and lumacaftor; and/or (b) at least one compound chosen from N-[2,4-bis(1,1-dimethylethyl)-5-hydroxyphenyl]-1,4-dihydro-4-oxoquinoline-3-carboxamide (ivacaftor), N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (deutivacaftor), (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol, deuterated derivatives of ivacaftor, deutivacaftor, and (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol, and pharmaceutically acceptable salts of any of the foregoing.

Another aspect of the disclosure provides methods of treating the CFTR-mediated disease cystic fibrosis comprising administering to a patient in need thereof at least one compound chosen from the novel compounds disclosed herein, pharmaceutically acceptable salts thereof, and deuterated derivatives of any of the foregoing, and optionally further administering one or more additional CFTR modulating agents selected from tezacaftor, ivacaftor, deutivacaftor, (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol, and lumacaftor.

In a further aspect, compounds of the disclosure (e.g., compounds of Formula I, compounds of any of Formulae Ia, IIa, IIb, III, IV, V, and VI, Compounds 1-508, tautomers thereof, deuterated derivatives of those compounds and tautomers, and pharmaceutically acceptable salts of any of the foregoing) and pharmaceutical compositions comprising those compounds, and optionally further comprising one or more CFTR modulating agents, are used in therapy or in the manufacture of a medicament. In some embodiments, the one or more additional CFTR modulating agents are selected from CFTR potentiators. In some embodiments, the one or more additional CFTR modulating agents are selected from CFTR correctors. In some embodiments, the one or more additional CFTR modulating agents are selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol, and deuterated derivatives and pharmaceutically acceptable salts of any of the foregoing.

A further aspect of the disclosure provides intermediates and methods for making the compounds and compositions disclosed herein.

### DEFINITIONS

“Tezacaftor,” as used herein, refers to (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide, which can be depicted with the following structure:

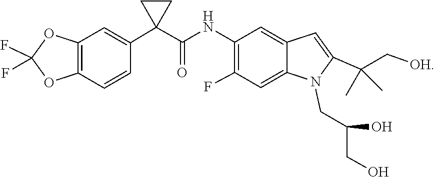 [SMILES: CC(C)(CO)c1cc2cc(NC(=O)C3(c4ccc5c(c4)OC(F)(F)O5)CC3)c(F)cc2n1C[C@@H](O)CO]

Tezacaftor may be in the form of a deuterated derivative, a pharmaceutically acceptable salt, or a pharmaceutically acceptable salt of a deuterated derivative. Tezacaftor and methods of making and using tezacaftor are disclosed in WO 2010/053471, WO 2011/119984, WO 2011/133751, WO 2011/133951, WO 2015/160787, and US 2009/0131492, each of which is incorporated herein by reference.

“Ivacaftor,” as used throughout this disclosure, refers to N-[2,4-bis(1,1-dimethylethyl)-5-hydroxyphenyl]-1,4-dihydro-4-oxoquinoline-3-carboxamide, which is depicted by the structure:

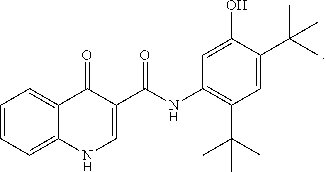 [SMILES: CC(C)(C)c1cc(C(C)(C)C)c(NC(=O)c2c[nH]c3ccccc3c2=O)cc1O]

Ivacaftor may also be in the form of a deuterated derivative, a pharmaceutically acceptable salt, or a pharmaceutically acceptable salt of a deuterated derivative. Ivacaftor and methods of making and using ivacaftor are disclosed in WO 2006/002421, WO 2007/079139, WO 2010/108162, and WO 2010/019239, each of which is incorporated herein by reference.

In some embodiments, a deuterated derivative of ivacaftor (deutivacaftor) is employed in the compositions and methods disclosed herein. A chemical name for deutivacaftor is N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide, as depicted by the structure:

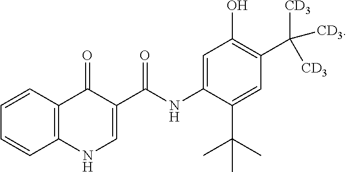 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

Deutivacaftor may be in the form of a further deuterated derivative, a pharmaceutically acceptable salt, or a pharmaceutically acceptable salt of a deuterated derivative. Deutivacaftor and methods of making and using deutivacaftor are disclosed in WO 2012/158885, WO 2014/078842, and U.S. Pat. No. 8,865,902, each of which is incorporated herein by reference.

“Lumacaftor” as used herein, refers to 3-(6-(1-(2,2-difluorobenzo[d][1,3]dioxol-5-yl)cyclopropanecarboxamido)-3-methylpyridin-2-yl)benzoic acid, which is depicted by the chemical structure:

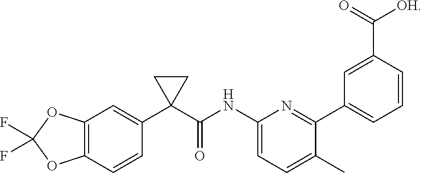 [SMILES: Cc1ccc(NC(=O)C2(c3ccc4c(c3)OC(F)(F)O4)CC2)nc1-c1cccc(C(=O)O)c1]

Lumacaftor may be in the form of a deuterated derivative, a pharmaceutically acceptable salt, or a pharmaceutically acceptable salt of a deuterated derivative. Lumacaftor and methods of making and using lumacaftor are disclosed in WO 2007/056341, WO 2009/073757, and WO 2009/076142, each of which is incorporated herein by reference.

As used herein, the term “alkyl” refers to a saturated or partially saturated, branched, or unbranched aliphatic hydrocarbon containing carbon atoms (such as, for example, 1, 2, 3, 4, 5, 6, 7, 8, 9, 10, 11, 12, 13, 14, 15, 16, 17, 18, 19, or 20 carbon atoms) in which one or more bonds between adjacent carbon atoms may be a double bond (alkenyl) or a triple bond (alkynyl). Alkyl groups may be substituted or unsubstituted.

As used herein, the term “haloalkyl group” refers to an alkyl group substituted with one or more halogen atoms, e.g., fluoroalkyl, which refers to an alkyl group substituted with one or more fluorine atoms.

The term “alkoxy,” as used herein, refers to an alkyl or cycloalkyl covalently bonded to an oxygen atom. Alkoxy groups may be substituted or unsubstituted.

As used herein, the term “haloalkoxyl group” refers to an alkoxy group substituted with one or more halogen atoms.

As used herein, “cycloalkyl” refers to a cyclic, bicyclic, tricyclic, or polycyclic non-aromatic hydrocarbon groups having 3 to 12 carbons (such as, for example 3-10 carbons) and may include one or more unsaturated bonds. “Cycloalkyl” groups encompass monocyclic, bicyclic, tricyclic, bridged, fused, and spiro rings, including mono spiro and dispiro rings. Non-limiting examples of cycloalkyl groups are cyclopropyl, cyclobutyl, cyclopentyl, cyclohexyl, adamantyl, norbornyl, dispiro[2.0.2.1]heptane, and spiro[2,3]hexane. Cycloalkyl groups may be substituted or unsubstituted.

The term “aryl,” as used herein, is a functional group or substituent derived from an aromatic ring and encompasses monocyclic aromatic rings and bicyclic, tricyclic, and fused ring systems wherein at least one ring in the system is aromatic. Non-limiting examples of aryl groups include phenyl, naphthyl, and 1,2,3,4-tetrahydronaphthalenyl.

The term “heteroaryl ring,” as used herein, refers to an aromatic ring comprising at least one ring atom that is a heteroatom, such as O, N, or S. Heteroaryl groups encompass monocyclic rings and bicyclic, tricyclic, bridged, fused, and spiro ring systems (including mono spiro and dispiro rings) wherein at least one ring in the system is aromatic. Non-limiting examples of heteroaryl rings include pyridine, quinoline, indole, and indoline.

As used herein, the term “heterocyclyl ring” refers to a non-aromatic hydrocarbon containing 3 to 12 atoms in a ring (such as, for example, 3-10 atoms) comprising at least one ring atom that is a heteroatom, such as O, N, or S, and may include one or more unsaturated bonds. “Heterocyclyl” rings encompass monocyclic, bicyclic, tricyclic, polycyclic, bridged, fused, and spiro rings, including mono spiro and dispiro rings.

“Substituted,” whether preceded by the term “optionally” or not, indicates that at least one hydrogen of the “substituted” group is replaced by a substituent. Unless otherwise indicated, an “optionally substituted” group may have a suitable substituent at each substitutable position of the group, and when more than one position in any given structure may be substituted with more than one substituent chosen from a specified group, the substituent may be either the same or different at each position.

Non-limiting examples of protecting groups for nitrogen include, for example, t-butyl carbamate (Boc), benzyl (Bn), para-methoxybenzyl (PMB), tetrahydropyranyl (THP), 9-fluorenylmethyl carbamate (Fmoc), benzyl carbamate (Cbz), methyl carbamate, ethyl carbamate, 2,2,2-trichloroethyl carbamate (Troc), 2-trimethylsilylethyl carbamate (Teoc), allyl carbamate (Aloc or Alloc), formamide, acetamide, benzamide, allylamine, trifluoroacetamide, triphenylmethylamine, benzylideneamine, and p-toluenesulfonamide. A comprehensive list of nitrogen protecting groups can be found in Wuts, P. G. M. “Greene's Protective Groups in Organic Synthesis: Fifth Edition,” 2014, John Wiley and Sons.

As used herein, “deuterated derivative(s)” refers to a compound having the same chemical structure as a reference compound, with one or more hydrogen atoms replaced by a deuterium atom. In some embodiments, the one or more hydrogens replaced by deuterium are part of an alkyl group. In some embodiments, the one or more hydrogens replaced by deuterium are part of a methyl group.

The phrase “and deuterated derivatives and pharmaceutically acceptable salts thereof” is used interchangeably with “and deuterated derivatives and pharmaceutically acceptable salts thereof of any of the forgoing” in reference to one or more specified compounds. These terms, as used herein, are intended to include deuterated derivatives of the specified compound or compounds and pharmaceutically acceptable salts of the specified compound or compounds, as well as pharmaceutically acceptable salts of deuterated derivatives of the specified compound or compounds.

As used herein, “CFTR” means cystic fibrosis transmembrane conductance regulator.

As used herein, the term “CFTR modulator” refers to a compound that increases the activity of CFTR. The increase in activity resulting from a CFTR modulator includes, but is not limited to, compounds that correct, potentiate, stabilize, and/or amplify CFTR.

As used interchangeably herein, the terms “CFTR corrector” or “corrector” refer to a compound that facilitates the processing and trafficking of CFTR to increase the amount of CFTR at the cell surface. The novel compounds disclosed herein are CFTR correctors. Tezacaftor, lumacaftor, and deuterated derivatives and pharmaceutically acceptable salts thereof, as referenced herein, are also CFTR correctors.

The terms “CFTR potentiator” and “potentiator,” as used interchangeably herein, refer to a compound that increases the channel activity of CFTR protein located at the cell surface, resulting in enhanced ion transport. Ivacaftor, deutivacaftor, (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol, and their deuterated derivatives and pharmaceutically acceptable salts are CFTR potentiators. It will be appreciated that when a description of a combination of compound selected from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, the combination will typically, but not necessarily, include a CFTR potentiator, such as, e.g., ivacaftor, deutivacaftor, (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol, or a deuterated derivative or pharmaceutically acceptable salt of any of the foregoing. In addition, the combination will typically, but not necessarily, include only a single potentiator, but may include more than a single corrector. Thus, in some embodiments, a combination of at least one compound selected from compounds of Formula I, compounds of any of Formulae Ia, IIa, IIb, III, IV, V, and VI, Compounds 1-508, tautomers thereof, deuterated derivatives of those compounds and tautomers, and pharmaceutically acceptable salts of any of the foregoing, will include a potentiator selected from ivacaftor, deutivacaftor, (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol, and deuterated derivatives and pharmaceutically acceptable salts thereof and may also include another CFTR corrector, such as, e.g., a corrector compound selected from tezacaftor, lumacaftor, and deuterated derivatives and pharmaceutically acceptable salts thereof.

The term “at least one compound selected from,” as used herein, refers to the selection of one or more of the compounds from a specified group.

A reference to “Compounds 1-508” in this disclosure is intended to represent a reference to each of Compounds 1 through 508 individually or a reference to groups of compounds, such as, e.g., Compounds 1-474, Compounds 475-506, and Compounds 507 and 508.

As used herein, the term “active pharmaceutical ingredient” or “therapeutic agent” (“API”) refers to a biologically active compound.

The terms “patient” and “subject” are used interchangeably and refer to an animal, including a human.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement in one or more symptoms of CF or lessening the severity of CF or one or more symptoms of CF in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in or lessening the severity of any of these symptoms can be readily assessed according to standard methods and techniques known in the art.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other.

It should be understood that references herein to methods of treatment (e.g., methods of treating a CFTR mediated disease or a method of treating cystic fibrosis) using one or more compounds of the disclosure optionally in combination with one or more additional CFTR modulating agents (e.g., a compound chosen from compounds of Formula I, compounds of any of Formulae Ia, IIa, IIb, III, IV, V, and VI, Compounds 1-508, tautomers thereof, deuterated derivatives of those compounds and tautomers, and pharmaceutically acceptable salts of any of the foregoing, optionally in combination with one or more additional CFTR modulating agents) should also be interpreted as references to:
one or more compounds (e.g., a compound chosen from compounds of Formula I, compounds of any of Formulae Ia, IIa, IIb, III, IV, V, and VI, Compounds 1-508, tautomers thereof, deuterated derivatives of those compounds and tautomers, and pharmaceutically acceptable salts of any of the foregoing, optionally in combination with one or more additional CFTR modulating agents) for use in methods of treating, e.g., cystic fibrosis, optionally in combination with one or more additional CFTR modulating agents; and/or
the use of one or more compounds (e.g., a compound chosen from compounds of Formula I, compounds of any of Formulae Ia, IIa, IIb, III, IV, V, and VI, Compounds 1-508, tautomers thereof, deuterated derivatives of those compounds and tautomers, and pharmaceutically acceptable salts of any of the foregoing, optionally in combination with one or more additional CFTR modulating agents) in the manufacture of a medicament for treating, e.g., cystic fibrosis.

It should be also understood that references herein to methods of treatment (e.g., methods of treating a CFTR mediated disease or a method of treating cystic fibrosis) using a pharmaceutical composition of the disclosure (e.g., a pharmaceutical composition comprising at least one compound chosen from compounds of Formula I, compounds of any of Formulae Ia, IIa, IIb, III, IV, V, and VI, Compounds 1-508, tautomers thereof, deuterated derivatives of those compounds and tautomers, and pharmaceutically acceptable salts of any of the foregoing, and optionally further comprising one or more additional CFTR modulating agents) should also be interpreted as references to:
a pharmaceutical composition (e.g., a pharmaceutical composition comprising at least one compound chosen from compounds of Formula I, compounds of any of Formulae Ia, IIa, IIb, III, IV, V, and VI, Compounds 1-508, tautomers thereof, deuterated derivatives of those compounds and tautomers, and pharmaceutically acceptable salts of any of the foregoing, and optionally further comprising one or more additional CFTR modulating agents) for use in methods of treating, e.g., cystic fibrosis; and/or
the use of a pharmaceutical composition (e.g., a pharmaceutical composition comprising at least one compound chosen from compounds of Formula I, compounds of any of Formulae Ia, IIa, IIb, III, IV, V, and VI, Compounds 1-508, tautomers thereof, deuterated derivatives of those compounds and tautomers, and pharmaceutically acceptable salts of any of the foregoing, and optionally further comprising one or more additional CFTR modulating agents) in the manufacture of a medicament for treating, e.g., cystic fibrosis.

The terms “about” and “approximately” may refer to an acceptable error for a particular value as determined by one of skill in the art, which depends in part on how the values are measured or determined. In some embodiments, the terms “about” and “approximately” mean within 20%, 15%, 10%, 5%, 4%, 3%, 2%, 1%, or 0.5% of a given value or range.

As used herein, the term “solvent” refers to any liquid in which the product is at least partially soluble (solubility of product >1 g/l).

As used herein, the term “room temperature” or “ambient temperature” means 15° C. to 30° C.

It will be appreciated that certain compounds of this disclosure may exist as separate stereoisomers or enantiomers and/or mixtures of those stereoisomers or enantiomers.

Certain compounds disclosed herein may exist as tautomers and both tautomeric forms are intended, even though only a single tautomeric structure is depicted. For example, a description of Compound X is understood to include its tautomer Compound Y and vice versa, as well as mixtures thereof:

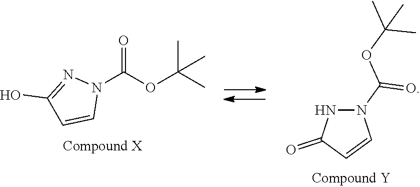

As used herein, “minimal function (MF) mutations” refer to CFTR gene mutations associated with minimal CFTR function (little-to-no functioning CFTR protein) and include, for example, mutations associated with severe defects in ability of the CFTR channel to open and close, known as defective channel gating or “gating mutations”; mutations associated with severe defects in the cellular processing of CFTR and its delivery to the cell surface; mutations associated with no (or minimal) CFTR synthesis; and mutations associated with severe defects in channel conductance.

As used herein, the term “pharmaceutically acceptable salt” refers to a salt form of a compound of this disclosure, wherein the salt is nontoxic. Pharmaceutically acceptable salts of the compounds of this disclosure include those derived from suitable inorganic and organic acids and bases. A “free base” form of a compound, for example, does not contain an ionically bonded salt.

The phrase “and deuterated derivatives and pharmaceutically acceptable salts thereof” is used interchangeably with “and deuterated derivatives and pharmaceutically acceptable salts thereof of any of the forgoing” in reference to one or more compounds or formulae of the disclosure. These phrases are intended to encompass pharmaceutically acceptable salts of any one of the referenced compounds, deuterated derivatives of any one of the referenced compounds, and pharmaceutically acceptable salts of those deuterated derivatives.

One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |
| | Triethiodide | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid, or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

The terms “selected from” and “chosen from” are used interchangeably herein.

### Methods of Treatment

Any of the novel compounds disclosed herein, such as, for example, compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, can act as a CFTR modulator, i.e., it modulates CFTR activity in the body. Individuals suffering from a mutation in the gene encoding CFTR may benefit from receiving a CFTR modulator. A CFTR mutation may affect the CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect). Some CFTR mutations exhibit characteristics of multiple classes. Certain mutations in the CFTR gene result in cystic fibrosis.

Thus, in some embodiments, the disclosure provides methods of treating, lessening the severity of, or symptomatically treating cystic fibrosis in a patient comprising administering to the patient an effective amount of any of the novel compounds disclosed herein, such as for example, compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, alone or in combination with another active ingredient, such as one or more CFTR modulating agents. In some embodiments, the one or more CFTR modulating agents are selected from ivacaftor, deutivacaftor, lumacaftor, and tezacaftor. In some embodiments, the patient has an F508del/minimal function (MF) genotype, F508del/F508del genotype (homozygous for the F508del mutation), F508del/gating genotype, or F508del/residual function (RF) genotype. In some embodiments, the patient is heterozygous and has one F508del mutation. In some embodiments, the patient is homozygous for the N1303K mutation.

In some embodiments, 5 mg to 500 mg of a compound disclosed herein, a tautomer thereof, a deuterated derivatives of the compound or tautomer, or a pharmaceutically acceptable salt of any of the foregoing are administered daily.

In some embodiments, the patient has at least one F508del mutation in the CFTR gene. In some embodiments, the patient has a CFTR gene mutation that is responsive to a compound, tautomer, deuterated derivative, or pharmaceutically acceptable salt of the disclosure based on in vitro data. In some embodiments, the patient is heterozygous and has an F508del mutation on one allele and a mutation on the other allele selected from Table 2:

TABLE 2
CFTR Mutations

| MF Category | Mutation | | | | |
|---|---|---|---|---|---|
| Nonsense mutations | Q2X | L218X | Q525X | R792X | E1104X |
| | S4X | Q220X | G542X | E822X | W1145X |
| | W19X | Y275X | G550X | W882X | R1158X |
| | G27X | C276X | Q552X | W846X | R1162X |
| | Q39X | Q290X | R553X | Y849X | S1196X |
| | W57X | G330X | E585X | R851X | W1204X |
| | E60X | W401X | G673X | Q890X | L1254X |
| | R75X | Q414X | Q685X | S912X | S1255X |
| | L88X | S434X | R709X | Y913X | W1282X |
| | E92X | S466X | K710X | Q1042X | Q1313X |
| | Q98X | S489X | Q715X | W1089X | Q1330X |
| | Y122X | Q493X | L732X | Y1092X | E1371X |
| | E193X | W496X | R764X | W1098X | Q1382X |
| | W216X | C524X | R785X | R1102X | Q1411X |
| Canonical splice | 185 + 1G→T | 711 + 5G→A | 1717 − 8G→A | 2622 + 1G→A | 3121 − 1G→A |
| mutations | 296 + 1G→A | 712 − 1G→T | 1717 − 1G→A | 2790 − 1G→C | 3500 − 2A→G |
| | 296 + 1G→T | 1248 + 1G→A | 1811 + 1G→C | 3040G→C (G970R) | 3600 + 2insT |
| | 405 + 1G→A | 1249 − 1G→A | 1811 + 1.6kbA→G | | 3850 − 1G→A |
| | 405 + 3A→C | 1341 + 1G→A | 1811 + 1643G→T | 3120G→A | 4005 + 1G→A |
| | 406 − 1G→A | 1525 − 2A→G | 1812 − 1G→A | 3120 + 1G→A | 4374 + 1G→T |
| | 621 + 1G→T | 1525 − 1G→A | 1898 + 1G→A | 3121 − 2A→G | |
| | 711 + 1G→T | | 1898 + 1G→C | | |
| Small (≤3 nucleotide) | 182delT | 1078delT | 1677delTA | 2711delT | 3737delA |
| insertion/deletion | 306insA | 1119delA | 1782delA | 2732insA | 3791delC |
| (ins/del) frameshift | 306delTAGA | 1138insG | 1824delA | 2869insG | 3821delT |
| mutations | 365-366insT | 1154insTC | 1833delT | 2896insAG | 3876delA |
| | 394delTT | 1161delC | 2043delG | 2942insT | 3878delG |
| | 442delA | 1213delT | 2143delT | 2957delT | 3905insT |
| | 444delA | 1259insA | 2183AA→G^{a} | 3007delG | 4016insT |
| | 457TAT→G | 1288insTA | 2184delA | 3028delA | 4021dupT |
| | 541delC | 1343delG | 2184insA | 3171delC | 4022insT |
| | 574delA | 1471delA | 2307insA | 3171insC | 4040delA |
| | 663delT | 1497delGG | 2347delG | 3271delGG | 4279insA |
| | 849delG | 1548delG | 2585delT | 3349insT | 4326delTC |
| | 935delA | 1609del CA | 2594delGT | 3659delC | |
| Non-small (>3 | CFTRdele1 | CFTRdele16-17b | 1461ins4 | | |
| nucleotide) | CFTRdele2 | CFTRdele17a,17b | 1924del7 | | |
| insertion/deletion | CFTRdele2,3 | CFTRdele17a-18 | 2055del9→A | | |
| (ins/del) frameshift | CFTRdele2-4 | CFTRdele19 | 2105-2117del13insAGAAA | | |
| mutations | CFTRdele3-10,14b-16 | CFTRdele19-21 | 2372del8 | | |
| | CFTRdele4-7 | CFTRdele21 | 2721del11 | | |
| | CFTRdele4-11 | CFTRdele22-24 | 2991del32 | | |
| | CFTR50kbdel | CFTRdele22,23 | 3667ins4 | | |
| | CFTRdup6b-10 | 124del23bp | 4010del4 | | |
| | CFTRdele11 | 602del14 | 4209TGTT→AA | | |
| | CFTRdele13,14a | 852del22 | | | |
| | CFTRdele14b-17b | 991del5 | | | |
| Missense mutations that | A46D | V520F | Y569D | N1303K | |
| Are not responsive in | G85E | A559T | L1065P | | |
| vitro to TEZ, IVA, or | R347P | R560T | R1066C | | |
| TEZ/IVA and | L467P | R250S | L1077P | | |
| % PI > 50% and | I507del | A561E | M1101K | | |
| SwCl⁻ > 86 mmol/L | | | | | |

^{a}Also known as 2183delAA→G.
CFTR: cystic fibrosis transmembrane conductance regulator;
IVA: ivacaftor.
SwCl: sweat chloride.
TEZ: tezacaftor.
Source: CFTR2.org [Internet]. Baltimore (MD): Clinical and functional translation of CFTR. The Clinical and Functional Translation of CFTR (CFTR2), US Cystic Fibrosis Foundation, Johns Hopkins University, the Hospital for Sick Children. Available at: http://www.cftr2.org/. Accessed 15 May 2018.
Notes:
% PI: percentage of F508del-CFTR heterozygous patients in the CFTR2 patient registry who are pancreatic insufficient; SwCl: mean sweat chloride of F508del-CFTR heterozygous patients in the CFTR2 patient registry.

In some embodiments, the disclosure also is directed to methods of treatment using isotope-labelled compounds of the afore-mentioned compounds, or pharmaceutically acceptable salts thereof, wherein the formula and variables of such compounds and salts are each and independently as described above or any other embodiments described above, provided that one or more atoms therein have been replaced by an atom or atoms having an atomic mass or mass number which differs from the atomic mass or mass number of the atom which usually occurs naturally (isotope labelled). Examples of isotopes which are commercially available and suitable for the disclosure include isotopes of hydrogen, carbon, nitrogen, oxygen, phosphorus, fluorine and chlorine, for example ²H, ³H, ¹³C, 14C, ¹⁵N, ¹⁸O, ¹⁷O, ³¹P, ³²P, ³⁵S, ¹⁸F and ³⁶Cl, respectively.

The isotope-labelled compounds and salts can be used in a number of beneficial ways. They can be suitable for medicaments and/or various types of assays, such as substrate tissue distribution assays. For example, tritium (³H)— and/or carbon-14 (¹⁴C)—labelled compounds are particularly useful for various types of assays, such as substrate tissue distribution assays, due to relatively simple preparation and excellent detectability. For example, deuterium (²H)—labelled ones are therapeutically useful with potential therapeutic advantages over the non-²H—labelled compounds. In general, deuterium (²H)—labelled compounds and salts can have higher metabolic stability as compared to those that are not isotope-labelled owing to the kinetic isotope effect described below. Higher metabolic stability translates directly into an increased in vivo half-life or lower dosages, which could be desired. The isotope-labelled compounds and salts can usually be prepared by carrying out the procedures disclosed in the synthesis schemes and the related description, in the example part and in the preparation part in the present text, replacing a non-isotope-labelled reactant by a readily available isotope-labelled reactant.

In some embodiments, the isotope-labelled compounds and salts are deuterium (²H)—labelled ones. In some embodiments, the isotope-labelled compounds and salts are deuterium (²H)—labelled, wherein one or more hydrogen atoms therein have been replaced by deuterium. In chemical structures, deuterium is represented as “D.”

The concentration of the isotope(s) (e.g., deuterium) incorporated into the isotope-labelled compounds and salts of the disclosure may be defined by the isotopic enrichment factor. The term “isotopic enrichment factor,” as used herein, means the ratio between the isotopic abundance and the natural abundance of a specified isotope. In some embodiments, if a substituent in a compound of the disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Combination Therapies

One aspect disclosed herein provides methods of treating cystic fibrosis and other CFTR mediated diseases using any of the novel compounds disclosed herein, such as, for example, compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, in combination with at least one additional active pharmaceutical ingredient.

In some embodiments, at least one additional active pharmaceutical ingredient is selected from mucolytic agents, bronchodilators, antibiotics, anti-infective agents, and anti-inflammatory agents.

In some embodiments, the additional therapeutic agent is an antibiotic. Exemplary antibiotics useful herein include tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.

In some embodiments, the additional agent is a mucolyte. Exemplary mucolytes useful herein includes Pulmozyme®.

In some embodiments, the additional agent is a bronchodilator. Exemplary bronchodilators include albuterol, metaprotenerol sulfate, pirbuterol acetate, salmeterol, or tetrabuline sulfate.

In some embodiments, the additional agent is an anti-inflammatory agent, i.e., an agent that can reduce the inflammation in the lungs. Exemplary such agents useful herein include ibuprofen, docosahexanoic acid (DHA), sildenafil, inhaled glutathione, pioglitazone, hydroxychloroquine, or simavastatin.

In some embodiments, the additional agent is a nutritional agent. Exemplary nutritional agents include pancrelipase (pancreatic enzyme replacement), including Pancrease®, Pancreacarb®, Ultrase®, or Creon®, Liprotomase® (formerly Trizytek®), Aquadeks®, or glutathione inhalation. In some embodiments, the additional nutritional agent is pancrelipase.

In some embodiments, at least one additional active pharmaceutical ingredient is selected from CFTR modulating agents. In some embodiments, the at least one additional active pharmaceutical ingredient is selected from CFTR potentiators. In some embodiments, the potentiator is selected from ivacaftor, deutivacaftor, (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol, and deuterated derivatives and pharmaceutically acceptable salts of any of the foregoing. In some embodiments, the at least one additional active pharmaceutical ingredient is chosen from CFTR correctors. In some embodiments, the correctors are selected from lumacaftor, tezacaftor, and deuterated derivatives and pharmaceutically acceptable salts of any of the foregoing.

In some embodiments, the at least one additional active pharmaceutical ingredient is chosen from (a) tezacaftor, lumacaftor, and deuterated derivatives and pharmaceutically acceptable salts thereof; and/or (b) ivacaftor, deutivacaftor, (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol, and deuterated derivatives and pharmaceutically acceptable salts of any of the foregoing.

Thus, in some embodiments, the combination therapies provided herein comprise (a) a compound selected from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing; and (b) at least one compound selected from tezacaftor, lumacaftor, and deuterated derivatives and pharmaceutically acceptable salts thereof; or (c) at least one compound selected from ivacaftor, deutivacaftor, and deuterated derivatives and pharmaceutically acceptable salts thereof. In other embodiments, the combination therapies provided herein comprise (a) at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing; (b) at least one compound selected from tezacaftor, lumacaftor, and deuterated derivatives and pharmaceutically acceptable salts thereof; and (c) at least one compound selected from ivacaftor, deutivacaftor, and deuterated derivatives and pharmaceutically acceptable salts thereof. In still other embodiments, the combination therapies provided herein comprise (a) at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing; (b) at least one compound selected from tezacaftor, lumacaftor, and deuterated derivatives and pharmaceutically acceptable salts thereof; and/or (c) at least one compound selected from (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol and deuterated derivatives and pharmaceutically acceptable salts thereof.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in combination with at least one compound chosen from tezacaftor and pharmaceutically acceptable salts thereof. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in combination with at least one compound chosen from lumacaftor and pharmaceutically acceptable salts thereof. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in combination with at least one compound chosen from ivacaftor and pharmaceutically acceptable salts thereof. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in combination with at least one compound chosen from deutivacaftor and pharmaceutically acceptable salts thereof. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in combination with at least one compound chosen from (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol and pharmaceutically acceptable salts thereof.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from ivacaftor and deuterated derivatives and pharmaceutically acceptable salts thereof. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from deutivacaftor and deuterated derivatives and pharmaceutically acceptable salts thereof. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from ivacaftor and deuterated derivatives and pharmaceutically acceptable salts thereof. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from deutivacaftor and deuterated derivatives and pharmaceutically acceptable salts thereof. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

Each of the compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, independently can be administered once daily, twice daily, or three times daily.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, tautomers thereof, deuterated derivatives of those compounds and tautomers, and pharmaceutically acceptable salts of any of the foregoing, is administered once daily. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered twice daily.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered once daily. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered twice daily.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, and at least one compound chosen from lumacaftor and deuterated derivatives and pharmaceutically acceptable salts thereof are administered once daily. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, and at least one compound chosen from lumacaftor and deuterated derivatives and pharmaceutically acceptable salts thereof are administered twice daily.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, and at least one compound chosen from ivacaftor, deutivacaftor, and deuterated derivatives and pharmaceutically acceptable salts thereof are administered once daily. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, and at least one compound chosen from ivacaftor, deutivacaftor, and deuterated derivatives and pharmaceutically acceptable salts thereof are administered twice daily.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, and at least one compound chosen from (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol and deuterated derivatives and pharmaceutically acceptable salts thereof are administered once daily. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, and at least one compound chosen from (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol and deuterated derivatives and pharmaceutically acceptable salts thereof are administered twice daily.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, at least one compound chosen from tezacaftor and deuterated derivatives and pharmaceutically acceptable salts thereof, and at least one compound chosen from ivacaftor, deutivacaftor, and deuterated derivatives and pharmaceutically acceptable salts thereof are administered once daily. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, at least one compound chosen from tezacaftor and deuterated derivatives and pharmaceutically acceptable salts thereof, and at least one compound chosen from ivacaftor, deutivacaftor, and deuterated derivatives and pharmaceutically acceptable salts thereof are administered twice daily.

Compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, at least one compound chosen from ivacaftor, deutivacaftor, and deuterated derivatives and pharmaceutically acceptable salts thereof, and at least one compound chosen from lumacaftor and deuterated derivatives and pharmaceutically acceptable salts thereof, are administered once daily. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, at least one compound chosen from ivacaftor, deutivacaftor, and deuterated derivatives and pharmaceutically acceptable salts thereof, and at least one compound chosen from lumacaftor and deuterated derivatives and pharmaceutically acceptable salts thereof, are administered twice daily.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, at least one compound chosen from tezacaftor, lumacaftor, and deuterated derivatives and pharmaceutically acceptable salts thereof, and at least one compound chosen from (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol and deuterated derivatives and pharmaceutically acceptable salts thereof, are administered once daily. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, at least one compound chosen from tezacaftor, lumacaftor, and deuterated derivatives and pharmaceutically acceptable salts thereof, and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof, are administered twice daily.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, and at least one compound chosen from tezacaftor and pharmaceutically acceptable salts thereof, are administered once daily and at least one compound chosen from ivacaftor and pharmaceutically acceptable salts thereof, are administered twice daily. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, and at least one compound chosen from lumacaftor and pharmaceutically acceptable salts thereof, are administered once daily and at least one compound chosen from ivacaftor and pharmaceutically acceptable salts thereof, are administered twice daily.

Compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, tezacaftor, ivacaftor, and deutivacaftor, and their pharmaceutically acceptable salts and deuterated derivatives thereof can be administered in a single pharmaceutical composition or separate pharmaceutical compositions. Such pharmaceutical compositions can be administered once daily or multiple times daily, such as twice daily. As used herein, the phrase that a given amount of API (e.g., tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol, or a deuterated derivative or a pharmaceutically acceptable salt thereof) is administered once or twice daily or per day means that said given amount is administered per dosing once or twice daily.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; and at least one compound chosen from ivacaftor and deuterated derivatives and pharmaceutically acceptable salts thereof is administered in a third pharmaceutical composition.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; at least one compound chosen from deutivacaftor and deuterated derivatives and pharmaceutically acceptable salts thereof is administered in a third pharmaceutical composition.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in a first pharmaceutical composition; at least one compound chosen from ivacaftor, deutivacaftor, and deuterated derivatives and pharmaceutically acceptable salts thereof is administered in a second pharmaceutical composition; at least one compound chosen from lumacaftor and deuterated derivatives and pharmaceutically acceptable salts thereof is administered in a third pharmaceutical composition.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in a first pharmaceutical composition; at least one compound chosen from tezacaftor, lumacaftor, and deuterated derivatives and pharmaceutically acceptable salts thereof is administered in a second pharmaceutical composition; at least one compound chosen from (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol and deuterated derivatives and pharmaceutically acceptable salts thereof is administered in a third pharmaceutical composition.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in a first pharmaceutical composition; and at least one compound chosen from tezacaftor and deuterated and pharmaceutically acceptable salts thereof and at least one compound chosen from ivacaftor, deutivacaftor, and deuterated derivatives and pharmaceutically acceptable salts thereof are administered in a second pharmaceutical composition. In some embodiments, the second pharmaceutical composition comprises a half of a daily dose of ivacaftor and the other half of the daily dose of ivacaftor is administered in a third pharmaceutical composition.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in a first pharmaceutical composition; and at least one compound chosen from lumacaftor and deuterated and pharmaceutically acceptable salts thereof and at least one compound chosen from ivacaftor, deutivacaftor, and deuterated derivatives and pharmaceutically acceptable salts thereof are administered in a second pharmaceutical composition. In some embodiments, the second pharmaceutical composition comprises a half of a daily dose of ivacaftor and the other half dose of ivacaftor is administered in a third pharmaceutical composition.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, is administered in a first pharmaceutical composition; and at least one compound chosen from tezacaftor, lumacaftor and deuterated and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol and deuterated and pharmaceutically acceptable salts thereof are administered in a second pharmaceutical composition.

In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing; at least one compound chosen from tezacaftor and pharmaceutically acceptable salts thereof and at least one compound chosen from ivacaftor, deutivacaftor, and deuterated derivatives and pharmaceutically acceptable salts thereof are administered in a first pharmaceutical composition. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing; at least one compound chosen from lumacaftor and pharmaceutically acceptable salts thereof and at least one compound chosen from ivacaftor, deutivacaftor, and deuterated derivatives and pharmaceutically acceptable salts thereof are administered in a first pharmaceutical composition. In some embodiments, at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing; at least one compound chosen from tezacaftor, lumacaftor, and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol and deuterated derivatives and pharmaceutically acceptable salts thereof are administered in a first pharmaceutical composition. In some embodiments, the first pharmaceutical composition is administered to the patient twice daily. In some embodiments, the first pharmaceutical composition is administered once daily. In some embodiments, the first pharmaceutical composition is administered once daily and, when the first pharmaceutical composition comprises ivacaftor, a second composition comprising only ivacaftor is administered once daily.

Any suitable pharmaceutical compositions can be used for compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing. Some exemplary pharmaceutical compositions for tezacaftor and its pharmaceutically acceptable salts can be found in WO 2011/119984 and WO 2014/014841, incorporated herein by reference. Some exemplary pharmaceutical compositions for ivacaftor and its pharmaceutically acceptable salts can be found in WO 2007/134279, WO 2010/019239, WO 2011/019413, WO 2012/027731, and WO 2013/130669, and some exemplary pharmaceutical compositions for deutivacaftor and its pharmaceutically acceptable salts can be found in U.S. Pat. Nos. 8,865,902, 9,181,192, 9,512,079, WO 2017/053455, and WO 2018/080591, all of which are incorporated herein by reference. Some exemplary pharmaceutical compositions for lumacaftor and its pharmaceutically acceptable salts can be found in WO 2010/037066, WO 2011/127421, and WO 2014/071122, incorporated herein by reference.

### Pharmaceutical Compositions

Another aspect of the disclosure provides a pharmaceutical composition comprising at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, and at least one pharmaceutically acceptable carrier.

In some embodiments, the disclosure provides pharmaceutical compositions comprising at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, in combination with at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator. In some embodiments, the pharmaceutical composition comprises at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, and at least two additional active pharmaceutical ingredients, one of which is a CFTR corrector and one of which is a CFTR potentiator.

In some embodiments, the disclosure provides a pharmaceutical composition comprising (a) at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, and (c) at least one pharmaceutically acceptable carrier.

In some embodiments, the disclosure provides a pharmaceutical composition comprising (a) at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, (b) at least one compound chosen from ivacaftor, deutivacaftor, and deuterated derivatives and pharmaceutically acceptable salts thereof, and (c) at least one pharmaceutically acceptable carrier.

In some embodiments, the disclosure provides a pharmaceutical composition comprising (a) at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, (b) at least one compound chosen from lumacaftor and deuterated derivatives and pharmaceutically acceptable salts thereof, and (c) at least one pharmaceutically acceptable carrier.

In some embodiments, the disclosure provides a pharmaceutical composition comprising (a) at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, (b) at least one compound chosen from (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol and deuterated derivatives and pharmaceutically acceptable salts thereof, and (c) at least one pharmaceutically acceptable carrier.

In some embodiments, the disclosure provides a pharmaceutical composition comprising (a) at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from ivacaftor and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from deutivacaftor and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, (b) at least one compound chosen from ivacaftor, deutivacaftor, and deuterated derivatives and pharmaceutically acceptable salts thereof, (c) at least one compound chosen from lumacaftor and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of Formulae Ia, IIa, IIb, III, IV, V, and VI, Compounds 1-508, tautomers thereof, deuterated derivatives of those compounds and tautomers, and pharmaceutically acceptable salts of any of the foregoing, (b) at least one compound chosen from tezacaftor, lumacaftor, and deuterated derivatives and pharmaceutically acceptable salts thereof, (c) at least one compound chosen from (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol and deuterated derivatives and pharmaceutically acceptable salts thereof, and (d) at least one pharmaceutically acceptable carrier.

Any pharmaceutical composition disclosed herein may comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, and lubricants.

The pharmaceutical compositions described herein are useful for treating cystic fibrosis and other CFTR mediated diseases.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York disclose various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### Exemplary Embodiments

The following provides a non-limiting list of exemplary embodiments:

1. A compound of Formula I:

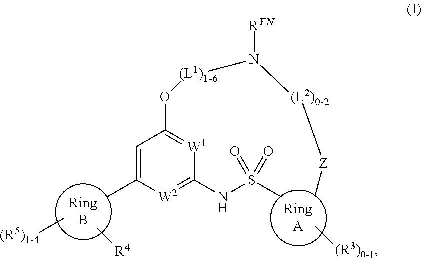

a tautomer thereof, a deuterated derivative of the compound or tautomer, or a pharmaceutically acceptable salt of any of the foregoing, wherein:
   Ring A is selected from:
      C₆-C₁₀ aryl,
      C₃-C₁₀ cycloalkyl,
      3- to 10-membered heterocyclyl, and
      5- to 10-membered heteroaryl;
   Ring B is selected from:
      C₆-C₁₀ aryl,
      C₃-C₁₀ cycloalkyl,
      3- to 10-membered heterocyclyl, and
      5- to 10-membered heteroaryl;
   V is selected from O and NH;
   W¹ is selected from N and CH;
   W² is selected from N and CH, provided that at least one of W¹ and W² is N;
   Z is selected from O, NR^{ZN}, and C(R^{ZC})₂, provided that when L² is absent, Z is C(R^{ZC})₂
   each L¹ is independently selected from C(R^{L1})₂ and

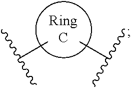

each L² is independently selected from C(R^{L2})₂;
Ring C is selected from C₆-C₁₀ aryl optionally substituted with 1-3 groups independently selected from:
   halogen,
   C₁-C₆ alkyl, and
   N(R^{N})₂;
each R³ is independently selected from:
   halogen,
   C₁-C₆ alkyl,
   C₁-C₆ alkoxy,
   C₃-C₁₀ cycloalkyl,
   C₆-C₁₀ aryl optionally substituted with 1-3 groups independently selected from C₁-C₆ alkyl, and
   3- to 10-membered heterocyclyl;
R⁴ is selected from hydrogen and C₁-C₆ alkyl;
each R⁵ is independently selected from:
   hydrogen,
   halogen,
   hydroxyl,
   N(R^{N})₂,
   —SO-Me,
   —CH═C(R^{LC})₂, wherein both R^{LC} are taken together to form a C₃-C₁₀ cycloalkyl,
   C₁-C₆ alkyl optionally substituted with 1-3 groups independently selected from:
      hydroxyl,
      C₁-C₆ alkoxy optionally substituted with 1-3 groups independently selected from C₁-C₆ alkoxy and C₆-C₁₀ aryl,
      C₃-C₁₀ cycloalkyl,
      —(O)₀₋₁—(C₆-C₁₀ aryl) optionally substituted with 1-3 groups independently selected from C₁-C₆ alkyl and C₁-C₆ alkoxy,
      3- to 10-membered heterocyclyl, and
      N(R^{N})₂,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl,
   C₆-C₁₀ aryl, and
   3- to 10-membered heterocyclyl;
R^{YN} is selected from:
   C₃-C₁₀ cycloalkyl optionally substituted with 1-3 groups independently selected from:
      hydroxyl,
      oxo,
      halogen,
      cyano,
      N(R^{N})₂,
      C₁-C₆ alkyl optionally substituted with 1-3 groups independently selected from:
      hydroxyl,
      oxo,
      N(R^{N})₂,
      C₁-C₆ alkoxy, and
      C₆-C₁₀ aryl,
      C₁-C₆ alkoxy optionally substituted with 1-3 groups independently selected from halogen, oxo, C₆-C₁₀ aryl, and N(R^{N})₂,
      halogen,
      C₃-C₁₀ cycloalkyl,
      3- to 10-memember heterocyclyl optionally substituted with 1-3 groups independently selected from C₁-C₆ alkyl, and
      5- to 10-membered heteroaryl optionally substituted with 1-3 groups independently selected from:
      hydroxyl,
      cyano,
      oxo,
      halogen,
      N(R^{N})₂,
      C₁-C₆ alkyl optionally substituted with 1-3 groups independently selected from hydroxyl, oxo, C₁-C₆ alkoxy, and N(R^{N})₂,
      C₁-C₆ alkoxy optionally substituted with 1-3 groups independently selected from hydroxyl, C₁-C₆ alkoxy, N(R^{N})₂, and C₃-C₁₀ cycloalkyl,
      C₁-C₆ fluoroalkyl,
      —(O)₀₋₁—(C₃-C₁₀ cycloalkyl) optionally substituted with 1-3 groups independently selected from C₁-C₆ alkyl,
      C₆-C₁₀ aryl, and
      3- to 10-membered heterocyclyl optionally substituted with 1-3 groups independently selected from C₁-C₆ alkyl,
   C₆-C₁₀ aryl,
   3- to 10-membered heterocyclyl optionally substituted with 1-3 groups independently selected from:
      oxo,
      C₁-C₆ alkyl optionally substituted with 1-3 groups independently selected from:
      oxo,
      hydroxyl,
      N(R^{N})₂,
      C₁-C₆ alkoxy optionally substituted with 1-3 groups independently selected from halogen and C₆-C₁₀ aryl, and
      —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl, and
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl);
R^{ZN} is selected from:
   hydrogen,
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
      hydroxyl,
      C₁-C₆ alkyl optionally substituted with 1-3 groups independently selected from hydroxyl, oxo, C₁-C₆ alkoxy, C₆-C₁₀ aryl, and N(R^{N})₂,
      C₁-C₆ fluoroalkyl,
      C₁-C₆ alkoxy,
      COOH,
      N(R^{N})₂,
      C₆-C₁₀ aryl, and
      3- to 10-membered heterocyclyl optionally substituted with 1-3 groups independently selected from oxo and C₁-C₆ alkyl,
      C₆-C₁₀ aryl optionally substituted with 1-3 groups independently selected from:
      halogen,
      hydroxyl,
      cyano,
      SiMe₃,
      SO₂Me,
      SF₅,
      N(R^{N})₂,
      P(O)Me₂,
      —(O)₀₋₁—(C₃-C₁₀ cycloalkyl) optionally substituted with 1-3 groups independently selected from C₁-C₆ fluoroalkyl,
      C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
      C₁-C₆ alkoxy optionally substituted with 1-3 groups independently selected from hydroxyl, oxo, N(R^{N})₂, and C₆-C₁₀ aryl,
      C₁-C₆ fluoroalkyl,
      3- to 10-membered heterocyclyl optionally substituted with 1-3 groups independently selected from C₁-C₆ alkyl,
      —(O)₀₋₁—(C₆-C₁₀ aryl), and
      —(O)₀₋₁—(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
      3- to 10-membered heterocyclyl optionally substituted with 1-4 groups independently selected from:
      hydroxyl,
      oxo,
      N(R^{N})₂,
      C₁-C₆ alkyl optionally substituted with 1-3 groups independently selected from oxo and C₁-C₆ alkoxy,
      C₁-C₆ alkoxy,
      C₁-C₆ fluoroalkyl,
      C₆-C₁₀ aryl optionally substituted with 1-3 groups independently selected from halogen, and
      5- to 10-membered heteroaryl, and
      5- to 10-membered heteroaryl optionally substituted with 1-3 groups independently selected from:
      hydroxyl,
      cyano,
      oxo,
      halogen,
      B(OH)_{2,}
      N(R^{N})₂,
      C₁-C₆ alkyl optionally substituted with 1-3 groups independently selected from hydroxyl, oxo, C₁-C₆ alkoxy (optionally substituted with 1-3 —SiMe₃), and N(R^{N})₂,
      C₁-C₆ alkoxy optionally substituted with 1-3 groups independently selected from hydroxyl, oxo, C₁-C₆ alkoxy, N(R^{N})₂, and C₃-C₁₀ cycloalkyl,
      C₁-C₆ fluoroalkyl,
      —(O)₀₋₁—(C₃-C₁₀ cycloalkyl) optionally substituted with 1-3 groups independently selected from C₁-C₆ alkyl,
      —(O)₀₋₁—(C₆-C₁₀ aryl),
      —(O)₀₋₁—(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, and 3- to 10-membered heterocyclyl (optionally substituted with 1-3 groups independently selected from C₁-C₆ fluoroalkyl), and
      5- to 10-membered heteroaryl optionally substituted with 1-4 groups independently selected from C₁-C₆ alkyl and C₃-C₁₀ cycloalkyl,
   C₁-C₆ fluoroalkyl,
   C₃-C₁₀ cycloalkyl optionally substituted with 1-3 groups independently selected from:
      hydroxyl,
      oxo,
      halogen,
      cyano,
      N(R^{N})₂,
      C₁-C₆ alkyl optionally substituted with 1-3 groups independently selected from:
      hydroxyl,
      oxo,
      N(R^{N})₂,
      C₁-C₆ alkoxy, and
      C₆-C₁₀ aryl,
      C₁-C₆ alkoxy optionally substituted with 1-3 groups independently selected from halogen, oxo, C₆-C₁₀ aryl, and N(R^{N})₂,
      halogen,
      C₃-C₁₀ cycloalkyl,
      3- to 10-memember heterocyclyl optionally substituted with 1-3 groups independently selected from C₁-C₆ alkyl, and
      5- to 10-membered heteroaryl optionally substituted with 1-3 groups independently selected from:
      hydroxyl,
      cyano,
      oxo,
      halogen,
      N(R^{N})₂,
      C₁-C₆ alkyl optionally substituted with 1-3 groups independently selected from hydroxyl, oxo, C₁-C₆ alkoxy, and N(R^{N})₂,
      C₁-C₆ alkoxy optionally substituted with 1-3 groups independently selected from hydroxyl, C₁-C₆ alkoxy, N(R^{N})₂, and C₃-C₁₀ cycloalkyl,
      C₁-C₆ fluoroalkyl,
      —(O)₀₋₁—(C₃-C₁₀ cycloalkyl) optionally substituted with 1-3 groups independently selected from C₁-C₆ alkyl,
      C₆-C₁₀ aryl, and
      3- to 10-membered heterocyclyl optionally substituted with 1-3 groups independently selected from C₁-C₆ alkyl,
   C₆-C₁₀ aryl,
   3- to 10-membered heterocyclyl optionally substituted with 1-3 groups independently selected from:
      oxo,
      C₁-C₆ alkyl optionally substituted with 1-3 groups independently selected from:
      oxo,
      hydroxyl,
      N(R^{N})₂,
      C₁-C₆ alkoxy optionally substituted with 1-3 groups independently selected from halogen and C₆-C₁₀ aryl, and
      —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl,
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
   R^{F};
each R^{ZC} is independently selected from:
   hydrogen,
   C₁-C₆ alkyl optionally substituted with 1-3 groups independently selected from C₆-C₁₀ aryl (optionally substituted with 1-3 groups independently selected from C₁-C₆ alkyl),
   C₆-C₁₀ aryl optionally substituted with 1-3 groups independently selected from C₁-C₆ alkyl, and
   R^{F};
or two R^{ZC} are taken together to form an oxo group;
each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen and C₁-C₆ fluoroalkyl,
      C₆-C₁₀ aryl optionally substituted with 1-3 groups independently selected from C₁-C₆ alkyl, and
      3- to 10-membered heterocyclyl optionally substituted with 1-3 groups independently selected from C₁-C₆ alkyl (optionally substituted with 1-3 groups independently selected from hydroxyl and oxo),
   C₃-C₁₀ cycloalkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from:
      hydroxyl,
      oxo,
      cyano,
      SiMe₃,
      N(R^{N})₂, and
      C₃-C₁₀ cycloalkyl optionally substituted with 1-3 groups independently selected from C₁-C₆ fluoroalkyl,
      C₁-C₆ alkoxy optionally substituted with 1-3 groups independently selected from:
      C₃-C₁₀ cycloalkyl optionally substituted with 1-3 groups independently selected from C₁-C₆ fluoroalkyl, and
      C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl,
      3- to 10-membered heterocyclyl optionally substituted with 1-3 groups independently selected from C₁-C₆ alkyl, and
      5- to 10-membered heteroaryl,
   3- to 10-membered heterocyclyl optionally substituted with 1-3 groups independently selected from:
      C₁-C₆ alkyl optionally substituted with 1-3 groups independently selected from:
      oxo, and
      C₁-C₆ alkoxy,
   5- to 10-membered heteroaryl optionally substituted with 1-3 groups independently selected from:
      C₁-C₆ alkyl optionally substituted with 1-3 groups independently selected from:
      C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl, and
   R^{F};
or two R^{L1} on the same carbon atom are taken together to form an oxo group;
each R^{L2} is independently selected from hydrogen and R^{F};
or two R^{L2} on the same carbon atom are taken together to form an oxo group;
each R^{N} is independently selected from:
   hydrogen,
   C₁-C₈ alkyl optionally substituted with 1-3 groups independently selected from:
      oxo,
      halogen,
      hydroxyl,
      NH₂,
      NHMe,
      NMe₂,
      NHCOMe,
      C₁-C₆ alkoxy optionally substituted with 1-3 groups independently selected from C₆-C₁₀ aryl,
      —(O)₀₋₁—(C₃-C₁₀ cycloalkyl),
      C₆-C₁₀ aryl optionally substituted with 1-3 groups independently selected from halogen and C₁-C₆ alkyl,
      3- to 14-membered heterocyclyl optionally substituted with 1-4 groups independently selected from oxo and C₁-C₆ alkyl, and
      5- to 14-membered heteroaryl optionally substituted with 1-4 groups independently selected from oxo and C₁-C₆ alkyl,
   C₃-C₁₀ cycloalkyl optionally substituted with 1-3 groups independently selected from:
      hydroxyl,
      NH₂,
      NHMe, and
      C₁-C₆ alkyl optionally substituted with 1-3 groups independently selected from hydroxyl,
   C₆-C₁₀ aryl, and
   3- to 10-membered heterocyclyl;
or two R^{N} on the same nitrogen atom are taken together with the nitrogen to which they are bonded to form a 3- to 10-membered heterocyclyl optionally substituted with 1-3 groups selected from:
   hydroxyl,
   oxo,
   cyano,
   C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
   C₁-C₆ alkoxy, and
   C₁-C₆ fluoroalkyl;
or one R⁴ and one R^{L1} are taken together to form a C₆-C₈ alkylene;
when R^{F} is present, two R^{F} taken together with the atoms to which they are bonded form a group selected from:
   C₃-C₁₀ cycloalkyl optionally substituted with 1-3 groups independently selected from C₁-C₆ alkyl,
   C₆-C₁₀ aryl optionally substituted with 1-3 groups independently selected from:
      halogen,
      C₁-C₆ alkyl,
      N(R^{N})₂, and
      3- to 10-membered heterocyclyl optionally substituted with 1-3 groups independently selected from hydroxyl,
   3- to 11-membered heterocyclyl optionally substituted with 1-3 groups independently selected from:
      oxo,
      N(R^{N})₂,
      C₁-C₉ alkyl optionally substituted with 1-4 groups independently selected from:
      oxo,
      halogen,
      hydroxyl,
      N(R^{N})₂,
      —SO₂—(C₁-C₆ alkyl),
      C₁-C₆ alkoxy optionally substituted with 1-3 groups independently selected from halogen and C₆-C₁₀ aryl,
      C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
      —(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
      3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N},
      —O-(5- to 12-membered heteroaryl) optionally substituted with 1-3 groups independently selected from C₆-C₁₀ aryl (optionally substituted with 1-3 groups independently selected from halogen) and C₁-C₆ alkyl, and
      5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and
      C₃-C₁₀ cycloalkyl,
      C₃-C₁₂ cycloalkyl optionally substituted with 1-4 groups independently selected from halogen, C₁-C₆ alkyl, and C₁-C₆ fluoroalkyl,
      C₆-C₁₀ aryl,
      3- to 10-membered heterocyclyl, and
      5- to 10-membered heteroaryl optionally substituted with 1-3 groups independently selected from C₁-C₆ alkoxy and C₁-C₆ fluoroalkyl, and
   5- to 12-membered heteroaryl optionally substituted with 1-3 groups independently selected from C₁-C₆ alkyl and C₁-C₆ fluoroalkyl;
with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.
   2. The compound, tautomer, deuterated derivative, or pharmaceutically acceptable salt according to embodiment 1, wherein Ring A is selected from C₆-C₁₀ aryl and 5- to 10-membered heteroaryl.
   3. The compound, tautomer, deuterated derivative, or pharmaceutically acceptable salt according to embodiment 1 or 2, wherein Ring A is selected from phenyl, pyridyl, pyrazinyl, and pyrazolyl.
   4. The compound, tautomer, deuterated derivative, or pharmaceutically acceptable salt according to any one of embodiments 1 to 3, wherein Ring A is phenyl.
   5. The compound, tautomer, deuterated derivative, or pharmaceutically acceptable salt according to any one of embodiments 1 to 4, wherein Ring B is selected from C₆-C₁₀ aryl and C₃-C₁₀ cycloalkyl.
   6. The compound, tautomer, deuterated derivative, or pharmaceutically acceptable salt according to any one of embodiments 1 to 5, wherein Ring B is selected from phenyl and cyclohexyl.
   7. The compound, tautomer, deuterated derivative, or pharmaceutically acceptable salt according to any one of embodiments 1 to 6, wherein Ring B is phenyl.
   8. The compound, tautomer, deuterated derivative, or pharmaceutically acceptable salt according to any one of embodiments 1 to 7, wherein V is O.
   9. The compound, tautomer, deuterated derivative, or pharmaceutically acceptable salt according to any one of embodiments 1 to 8, wherein W¹ is N and W² is N.
   10. The compound, tautomer, deuterated derivative, or pharmaceutically acceptable salt according to any one of embodiments 1 to 8, wherein W¹ is CH and W² is N.
   11. The compound, tautomer, deuterated derivative, or pharmaceutically acceptable salt according to any one of embodiments 1 to 10, wherein Z is C(R^{ZC}).
   12. The compound, tautomer, deuterated derivative, or pharmaceutically acceptable salt according to any one of embodiments 1 to 11, wherein two R^{ZC} are taken together to form an oxo group.
   13. The compound, tautomer, deuterated derivative, or pharmaceutically acceptable salt according to any one of embodiments 1 to 12, wherein each R³ is independently selected from C₁-C₆ alkyl.
   14. The compound, tautomer, deuterated derivative, or pharmaceutically acceptable salt according to any one of embodiments 1 to 13, wherein each R³ is methyl.
   15. The compound, tautomer, deuterated derivative, or pharmaceutically acceptable salt according to any one of embodiments 1 to 12, wherein R³ is absent.
   16. The compound, tautomer, deuterated derivative, or pharmaceutically acceptable salt according to any one of embodiments 1 to 15, wherein R⁴ is selected from hydrogen and methyl.
   17. The compound, tautomer, deuterated derivative, or pharmaceutically acceptable salt according to any one of embodiments 1 to 16, wherein R⁴ is methyl.
   18. The compound, tautomer, deuterated derivative, or pharmaceutically acceptable salt according to any one of embodiments 1 to 17, wherein each R⁵ is independently selected from hydrogen, halogen, C₁-C₆ alkyl, C₁-C₆ fluoroalkyl, and C₆-C₁₀ aryl.
   19. The compound, tautomer, deuterated derivative, or pharmaceutically acceptable salt according to any one of embodiments 1 to 18, wherein R^{YN} is selected from:
   C₃-C₁₀ cycloalkyl optionally substituted with 1-3 groups independently selected from:
      hydroxyl,
      cyano,
      N(R^{N})₂,
      C₁-C₆ alkyl optionally substituted with 1-3 groups independently selected from:
         hydroxyl,
         oxo,
         N(R^{N})₂, and
         C₆-C₁₀ aryl,
      C₁-C₆ alkoxy optionally substituted with 1-3 groups independently selected from halogen, oxo, C₆-C₁₀ aryl, and N(R^{N})₂,
      C₃-C₁₀ cycloalkyl, and
      5- to 10-membered heteroaryl optionally substituted with 1-3 groups independently selected from:
         hydroxyl,
         oxo,
         N(R^{N})₂,
         C₁-C₆ alkyl optionally substituted with 1-3 groups independently selected from C₁-C₆ alkoxy, and
         C₁-C₆ alkoxy optionally substituted with 1-3 groups independently selected from C₃-C₁₀ cycloalkyl,
   3- to 10-membered heterocyclyl optionally substituted with 1-3 groups independently selected from:
      C₁-C₆ alkyl optionally substituted with 1-3 groups independently selected from:
         oxo,
         hydroxyl,
         N(R^{N})₂,
         C₁-C₆ alkoxy optionally substituted with 1-3 groups independently selected from C₆-C₁₀ aryl, and
         —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl, and
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl).
         20. The compound, tautomer, deuterated derivative, or pharmaceutically acceptable salt according to any one of embodiments 1 to 19, wherein each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl, and
      C₃-C₁₀ cycloalkyl optionally substituted with 1-3 groups independently selected from halogen and C₁-C₆ fluoroalkyl, and
   C₃-C₁₀ cycloalkyl.
      21. The compound, tautomer, deuterated derivative, or pharmaceutically acceptable salt according to any one of embodiments 1 to 20, wherein each R^{N} is independently selected from:
   hydrogen,
   C₁-C₈ alkyl optionally substituted with 1-3 groups independently selected from:
      NH₂,
      NHCOMe,
      C₁-C₆ alkoxy,
      —(O)₀₋₁—(C₃-C₁₀ cycloalkyl),
      C₆-C₁₀ aryl, and
      3- to 14-membered heterocyclyl optionally substituted with 1-4 groups independently selected from C₁-C₆ alkyl, and
   C₆-C₁₀ aryl, and
   or two R^{N} on the same nitrogen atom are taken together with the nitrogen to which they are bonded to form a 3- to 10-membered heterocyclyl optionally substituted with 1-3 groups selected from:
   cyano,
   C₁-C₆ alkyl, and
   C₁-C₆ alkoxy.
      22. A compound of Formula Ia:

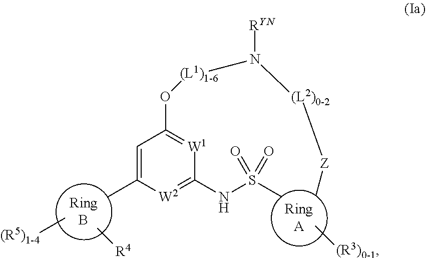

a tautomer thereof, a deuterated derivative of the compound or tautomer, or a pharmaceutically acceptable salt of any of the foregoing, wherein Ring A, Ring B, W¹, W², Z, L¹, L², R³, R⁴, R⁵, and R^{YN} are defined as according to embodiment 1, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.
   23. The compound, tautomer, deuterated derivative, or pharmaceutically acceptable salt according to embodiment 22, wherein Ring A is selected from C₆-C₁₀ aryl and 5- to 10-membered heteroaryl.
   24. The compound, tautomer, deuterated derivative, or pharmaceutically acceptable salt according to embodiment 22 or 23, wherein Ring A is selected from phenyl, pyridyl, pyrazinyl, and pyrazolyl.
   25. The compound, tautomer, deuterated derivative, or pharmaceutically acceptable salt according to any one of embodiments 22 to 24, wherein Ring A is phenyl.
   26. The compound, tautomer, deuterated derivative, or pharmaceutically acceptable salt according to any one of embodiments 22 to 25, wherein Ring B is selected from C₆-C₁₀ aryl and C₃-C₁₀ cycloalkyl.
   27. The compound, tautomer, deuterated derivative, or pharmaceutically acceptable salt according to any one of embodiments 22 to 26, wherein Ring B is selected from phenyl and cyclohexyl.
   28. The compound, tautomer, deuterated derivative, or pharmaceutically acceptable salt according to any one of embodiments 22 to 27, wherein Ring B is phenyl.
   29. The compound, tautomer, deuterated derivative, or pharmaceutically acceptable salt according to any one of embodiments 22 to 28, wherein W¹ is N and W² is N.
   30. The compound, tautomer, deuterated derivative, or pharmaceutically acceptable salt according to any one of embodiments 22 to 29, wherein W¹ is CH and W² is N.
   31. The compound, tautomer, deuterated derivative, or pharmaceutically acceptable salt according to any one of embodiments 22 to 30, wherein Z is C(R^{ZC}).
   32. The compound, tautomer, deuterated derivative, or pharmaceutically acceptable salt according to any one of embodiments 22 to 31, wherein two R^{ZC} are taken together to form an oxo group.
   33. The compound, tautomer, deuterated derivative, or pharmaceutically acceptable salt according to any one of embodiments 22 to 32, wherein each R³ is independently selected from C₁-C₆ alkyl.
   34. The compound, tautomer, deuterated derivative, or pharmaceutically acceptable salt according to any one of embodiments 22 to 33, wherein each R³ is methyl.
   35. The compound, tautomer, deuterated derivative, or pharmaceutically acceptable salt according to any one of embodiments 22 to 32, wherein R³ is absent.
   36. The compound, tautomer, deuterated derivative, or pharmaceutically acceptable salt according to any one of embodiments 22 to 35, wherein R⁴ is selected from hydrogen and methyl.
   37. The compound, tautomer, deuterated derivative, or pharmaceutically acceptable salt according to any one of embodiments 22 to 36, wherein R⁴ is methyl.
   38. The compound, tautomer, deuterated derivative, or pharmaceutically acceptable salt according to any one of embodiments 22 to 37, wherein each R⁵ is independently selected from hydrogen, halogen, C₁-C₆ alkyl, C₁-C₆ fluoroalkyl, and C₆-C₁₀ aryl.
   39. The compound, tautomer, deuterated derivative, or pharmaceutically acceptable salt according to any one of embodiments 22 to 38, wherein R^{YN} is selected from:
   C₃-C₁₀ cycloalkyl optionally substituted with 1-3 groups independently selected from:
      hydroxyl,
      cyano,
      N(R^{N})₂,
      C₁-C₆ alkyl optionally substituted with 1-3 groups independently selected from:
         hydroxyl,
         oxo,
         N(R^{N})₂, and
         C₆-C₁₀ aryl,
      C₁-C₆ alkoxy optionally substituted with 1-3 groups independently selected from halogen, oxo, C₆-C₁₀ aryl, and N(R^{N})₂,
      C₃-C₁₀ cycloalkyl, and
      5- to 10-membered heteroaryl optionally substituted with 1-3 groups independently selected from:
         hydroxyl,
         oxo,
         N(R^{N})₂,
         C₁-C₆ alkyl optionally substituted with 1-3 groups independently selected from C₁-C₆ alkoxy, and
         C₁-C₆ alkoxy optionally substituted with 1-3 groups independently selected from C₃-C₁₀ cycloalkyl,
   3- to 10-membered heterocyclyl optionally substituted with 1-3 groups independently selected from:
      C₁-C₆ alkyl optionally substituted with 1-3 groups independently selected from:
         oxo,
         hydroxyl,
         N(R^{N})₂,
         C₁-C₆ alkoxy optionally substituted with 1-3 groups independently selected from C₆-C₁₀ aryl, and
         —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl, and
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl).
         40. The compound, tautomer, deuterated derivative, or pharmaceutically acceptable salt according to any one of embodiments 22 to 39, wherein each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl, and
      C₃-C₁₀ cycloalkyl optionally substituted with 1-3 groups independently selected from halogen and C₁-C₆ fluoroalkyl, and
   C₃-C₁₀ cycloalkyl.
      41. The compound, tautomer, deuterated derivative, or pharmaceutically acceptable salt according to any one of embodiments 22 to 40, wherein each R^{N} is independently selected from:
   hydrogen,
   C₁-C₈ alkyl optionally substituted with 1-3 groups independently selected from:
      NH₂,
      NHCOMe,
      C₁-C₆ alkoxy,
      —(O)₀₋₁—(C₃-C₁₀ cycloalkyl),
      C₆-C₁₀ aryl, and
      3- to 14-membered heterocyclyl optionally substituted with 1-4 groups independently selected from C₁-C₆ alkyl, and
   C₆-C₁₀ aryl, and
   or two R^{N} on the same nitrogen atom are taken together with the nitrogen to which they are bonded to form a 3- to 10-membered heterocyclyl optionally substituted with 1-3 groups selected from:
   cyano,
   C₁-C₆ alkyl, and
   C₁-C₆ alkoxy.
      42. A compound of Formula IIa:

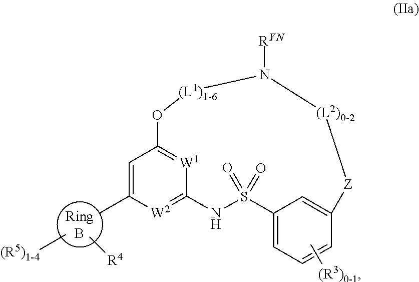

a tautomer thereof, a deuterated derivative of the compound or tautomer, or a pharmaceutically acceptable salt of any of the foregoing, wherein Ring B, W¹, W², Z, L¹, L², R³, R⁴, R⁵, and R^{YN} are defined as according to embodiment 1, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.
   43. The compound, tautomer, deuterated derivative, or pharmaceutically acceptable salt according to embodiment 42, wherein Ring B is selected from C₆-C₁₀ aryl and C₃-C₁₀ cycloalkyl.
   44. The compound, tautomer, deuterated derivative, or pharmaceutically acceptable salt according to embodiment 42 or 43, wherein Ring B is selected from phenyl and cyclohexyl.
   45. The compound, tautomer, deuterated derivative, or pharmaceutically acceptable salt according to any one of embodiments 42 to 44, wherein Ring B is phenyl.
   46. The compound, tautomer, deuterated derivative, or pharmaceutically acceptable salt according to any one of embodiments 42 to 45, wherein W¹ is N and W² is N.
   47. The compound, tautomer, deuterated derivative, or pharmaceutically acceptable salt according to any one of embodiments 42 to 46, wherein W¹ is CH and W² is N.
   48. The compound, tautomer, deuterated derivative, or pharmaceutically acceptable salt according to any one of embodiments 42 to 47, wherein Z is C(R^{ZC}).
   49. The compound, tautomer, deuterated derivative, or pharmaceutically acceptable salt according to any one of embodiments 42 to 48, wherein two R^{ZC} are taken together to form an oxo group.
   50. The compound, tautomer, deuterated derivative, or pharmaceutically acceptable salt according to any one of embodiments 42 to 49, wherein each R³ is independently selected from C₁-C₆ alkyl.
   51. The compound, tautomer, deuterated derivative, or pharmaceutically acceptable salt according to any one of embodiments 42 to 50, wherein each R³ is methyl.
   52. The compound, tautomer, deuterated derivative, or pharmaceutically acceptable salt according to any one of embodiments 42 to 49, wherein R³ is absent.
   53. The compound, tautomer, deuterated derivative, or pharmaceutically acceptable salt according to any one of embodiments 42 to 52, wherein R⁴ is selected from hydrogen and methyl.
   54. The compound, tautomer, deuterated derivative, or pharmaceutically acceptable salt according to any one of embodiments 42 to 53, wherein R⁴ is methyl.
   55. The compound, tautomer, deuterated derivative, or pharmaceutically acceptable salt according to any one of embodiments 42 to 54, wherein each R⁵ is independently selected from hydrogen, halogen, C₁-C₆ alkyl, C₁-C₆ fluoroalkyl, and C₆-C₁₀ aryl.
   56. The compound, tautomer, deuterated derivative, or pharmaceutically acceptable salt according to any one of embodiments 42 to 55, wherein R^{YN} is selected from:
   C₃-C₁₀ cycloalkyl optionally substituted with 1-3 groups independently selected from:
      hydroxyl,
      cyano,
      N(R^{N})₂,
      C₁-C₆ alkyl optionally substituted with 1-3 groups independently selected from:
         hydroxyl,
         oxo,
         N(R^{N})₂, and
         C₆-C₁₀ aryl,
      C₁-C₆ alkoxy optionally substituted with 1-3 groups independently selected from halogen, oxo, C₆-C₁₀ aryl, and N(R^{N})₂,
      C₃-C₁₀ cycloalkyl, and
      5- to 10-membered heteroaryl optionally substituted with 1-3 groups independently selected from:
         hydroxyl,
         oxo,
         N(R^{N})₂,
         C₁-C₆ alkyl optionally substituted with 1-3 groups independently selected from C₁-C₆ alkoxy, and
         C₁-C₆ alkoxy optionally substituted with 1-3 groups independently selected from C₃-C₁₀ cycloalkyl,
   3- to 10-membered heterocyclyl optionally substituted with 1-3 groups independently selected from:
      C₁-C₆ alkyl optionally substituted with 1-3 groups independently selected from:
         oxo,
         hydroxyl,
         N(R^{N})₂,
         C₁-C₆ alkoxy optionally substituted with 1-3 groups independently selected from C₆-C₁₀ aryl, and
         —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl, and
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl).
         57. The compound, tautomer, deuterated derivative, or pharmaceutically acceptable salt according to any one of embodiments 42 to 56, wherein each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl, and
      C₃-C₁₀ cycloalkyl optionally substituted with 1-3 groups independently selected from halogen and C₁-C₆ fluoroalkyl, and
   C₃-C₁₀ cycloalkyl.
      58. The compound, tautomer, deuterated derivative, or pharmaceutically acceptable salt according to any one of embodiments 42 to 57, wherein each R^{N} is independently selected from:
   hydrogen,
   C₁-C₈ alkyl optionally substituted with 1-3 groups independently selected from:
      NH₂,
      NHCOMe,
      C₁-C₆ alkoxy,
      —(O)₀₋₁—(C₃-C₁₀ cycloalkyl),
      C₆-C₁₀ aryl, and
      3- to 14-membered heterocyclyl optionally substituted with 1-4 groups independently selected from C₁-C₆ alkyl, and
   C₆-C₁₀ aryl,
   or two R^{N} on the same nitrogen atom are taken together with the nitrogen to which they are bonded to form a 3- to 10-membered heterocyclyl optionally substituted with 1-3 groups selected from:
   cyano,
   C₁-C₆ alkyl, and
   C₁-C₆ alkoxy.
      59. A compound of Formula IIb:

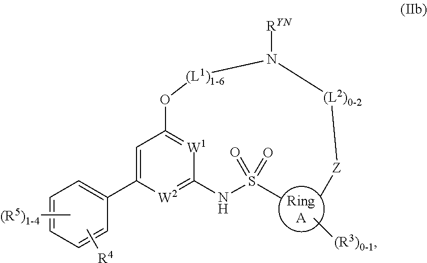

a tautomer thereof, a deuterated derivative of the compound or tautomer, or a pharmaceutically acceptable salt of any of the foregoing, wherein Ring A, W¹, W², Z, L¹, L², R³, R⁴, R⁵, and R^{YN} are defined as according to embodiment 1, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.
   60. The compound, tautomer, deuterated derivative, or pharmaceutically acceptable salt according to embodiment 59, wherein Ring A is selected from C₆-C₁₀ aryl and 5- to 10-membered heteroaryl.
   61. The compound, tautomer, deuterated derivative, or pharmaceutically acceptable salt according to embodiment 59 or 60, wherein Ring A is selected from phenyl, pyridyl, pyrazinyl, and pyrazolyl.
   62. The compound, tautomer, deuterated derivative, or pharmaceutically acceptable salt according to any one of embodiments 59 to 61, wherein Ring A is phenyl.
   63. The compound, tautomer, deuterated derivative, or pharmaceutically acceptable salt according to any one of embodiments 59 to 62, wherein W¹ is N and W² is N.
   64. The compound, tautomer, deuterated derivative, or pharmaceutically acceptable salt according to any one of embodiments 59 to 63, wherein W¹ is CH and W² is N.
   65. The compound, tautomer, deuterated derivative, or pharmaceutically acceptable salt according to any one of embodiments 59 to 64, wherein Z is C(R^{ZC}).
   66. The compound, tautomer, deuterated derivative, or pharmaceutically acceptable salt according to any one of embodiments 59 to 65, wherein two R^{ZC} are taken together to form an oxo group.
   67. The compound, tautomer, deuterated derivative, or pharmaceutically acceptable salt according to any one of embodiments 59 to 66, wherein each R³ is independently selected from C₁-C₆ alkyl.
   68. The compound, tautomer, deuterated derivative, or pharmaceutically acceptable salt according to any one of embodiments 59 to 67, wherein each R³ is methyl.
   69. The compound, tautomer, deuterated derivative, or pharmaceutically acceptable salt according to any one of embodiments 59 to 66, wherein R³ is absent.
   70. The compound, tautomer, deuterated derivative, or pharmaceutically acceptable salt according to any one of embodiments 59 to 69, wherein R⁴ is selected from hydrogen and methyl.
   71. The compound, tautomer, deuterated derivative, or pharmaceutically acceptable salt according to any one of embodiments 59 to 70, wherein R⁴ is methyl.
   72. The compound, tautomer, deuterated derivative, or pharmaceutically acceptable salt according to any one of embodiments 59 to 71, wherein each R⁵ is independently selected from hydrogen, halogen, C₁-C₆ alkyl, C₁-C₆ fluoroalkyl, and C₆-C₁₀ aryl.
   73. The compound, tautomer, deuterated derivative, or pharmaceutically acceptable salt according to any one of embodiments 59 to 72, wherein R^{YN} is selected from:
   C₃-C₁₀ cycloalkyl optionally substituted with 1-3 groups independently selected from:
      hydroxyl,
      cyano,
      N(R^{N})₂,
      C₁-C₆ alkyl optionally substituted with 1-3 groups independently selected from:
         hydroxyl,
         oxo,
         N(R^{N})₂, and
         C₆-C₁₀ aryl,
      C₁-C₆ alkoxy optionally substituted with 1-3 groups independently selected from halogen, oxo, C₆-C₁₀ aryl, and N(R^{N})₂,
      C₃-C₁₀ cycloalkyl, and
      5- to 10-membered heteroaryl optionally substituted with 1-3 groups independently selected from:
         hydroxyl,
         oxo,
         N(R^{N})₂,
         C₁-C₆ alkyl optionally substituted with 1-3 groups independently selected from C₁-C₆ alkoxy, and
         C₁-C₆ alkoxy optionally substituted with 1-3 groups independently selected from C₃-C₁₀ cycloalkyl,
   3- to 10-membered heterocyclyl optionally substituted with 1-3 groups independently selected from:
      C₁-C₆ alkyl optionally substituted with 1-3 groups independently selected from:
         oxo,
         hydroxyl,
         N(R^{N})₂,
         C₁-C₆ alkoxy optionally substituted with 1-3 groups independently selected from C₆-C₁₀ aryl, and
         —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl, and
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl).
         74. The compound, tautomer, deuterated derivative, or pharmaceutically acceptable salt according to any one of embodiments 59 to 73, wherein each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl, and
      C₃-C₁₀ cycloalkyl optionally substituted with 1-3 groups independently selected from halogen and C₁-C₆ fluoroalkyl, and
   C₃-C₁₀ cycloalkyl.
      75. The compound, tautomer, deuterated derivative, or pharmaceutically acceptable salt according to any one of embodiments 59 to 74, wherein each R^{N} is independently selected from:
   hydrogen,
   C₁-C₈ alkyl optionally substituted with 1-3 groups independently selected from:
      NH₂,
      NHCOMe,
      C₁-C₆ alkoxy,
      —(O)₀₋₁—(C₃-C₁₀ cycloalkyl),
      C₆-C₁₀ aryl, and
      3- to 14-membered heterocyclyl optionally substituted with 1-4 groups independently selected from C₁-C₆ alkyl,
   C₆-C₁₀ aryl, and
   or two R^{N} on the same nitrogen atom are taken together with the nitrogen to which they are bonded to form a 3- to 10-membered heterocyclyl optionally substituted with 1-3 groups selected from:
   cyano,
   C₁-C₆ alkyl, and
   C₁-C₆ alkoxy.
      76. A compound of Formula III:

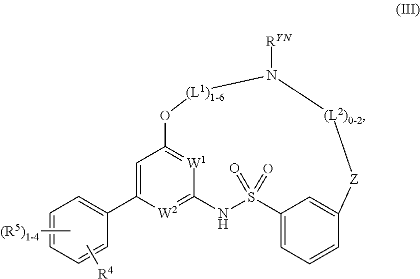

a tautomer thereof, a deuterated derivative of the compound or tautomer, or a pharmaceutically acceptable salt of any of the foregoing, wherein W¹, W², Z, L¹, L², R⁴, R⁵, and R^{YN} are defined as according to embodiment 1, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.
   77. The compound, tautomer, deuterated derivative, or pharmaceutically acceptable salt according to embodiment 76, wherein W¹ is N and W² is N.
   78. The compound, tautomer, deuterated derivative, or pharmaceutically acceptable salt according to embodiment 76 or 77, wherein W¹ is CH and W² is N.
   79. The compound, tautomer, deuterated derivative, or pharmaceutically acceptable salt according to any one of embodiments 76 to 78, wherein Z is C(R^{ZC}).
   80. The compound, tautomer, deuterated derivative, or pharmaceutically acceptable salt according to any one of embodiments 76 to 79, wherein two R^{ZC} are taken together to form an oxo group.
   81. The compound, tautomer, deuterated derivative, or pharmaceutically acceptable salt according to any one of embodiments 76 to 80, wherein R⁴ is selected from hydrogen and methyl.
   82. The compound, tautomer, deuterated derivative, or pharmaceutically acceptable salt according to any one of embodiments 76 to 81, wherein R⁴ is methyl.
   83. The compound, tautomer, deuterated derivative, or pharmaceutically acceptable salt according to any one of embodiments 76 to 82, wherein each R⁵ is independently selected from hydrogen, halogen, C₁-C₆ alkyl, C₁-C₆ fluoroalkyl, and C₆-C₁₀ aryl.
   84. The compound, tautomer, deuterated derivative, or pharmaceutically acceptable salt according to any one of embodiments 76 to 83, wherein R^{YN} is selected from:
   C₃-C₁₀ cycloalkyl optionally substituted with 1-3 groups independently selected from:
      hydroxyl,
      cyano,
      N(R^{N})₂,
      C₁-C₆ alkyl optionally substituted with 1-3 groups independently selected from:
         hydroxyl,
         oxo,
         N(R^{N})₂, and
      C₆-C₁₀ aryl,
      C₁-C₆ alkoxy optionally substituted with 1-3 groups independently selected from halogen, oxo, C₆-C₁₀ aryl, and N(R^{N})₂,
      C₃-C₁₀ cycloalkyl, and
      5- to 10-membered heteroaryl optionally substituted with 1-3 groups independently selected from:
         hydroxyl,
         oxo,
         N(R^{N})₂,
         C₁-C₆ alkyl optionally substituted with 1-3 groups independently selected from C₁-C₆ alkoxy, and
         C₁-C₆ alkoxy optionally substituted with 1-3 groups independently selected from C₃-C₁₀ cycloalkyl,
   3- to 10-membered heterocyclyl optionally substituted with 1-3 groups independently selected from:
      C₁-C₆ alkyl optionally substituted with 1-3 groups independently selected from:
         oxo,
         hydroxyl,
         N(R^{N})₂,
         C₁-C₆ alkoxy optionally substituted with 1-3 groups independently selected from C₆-C₁₀ aryl, and
         —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl, and
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl).
         85. The compound, tautomer, deuterated derivative, or pharmaceutically acceptable salt according to any one of embodiments 76 to 84, wherein each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl, and
      C₃-C₁₀ cycloalkyl optionally substituted with 1-3 groups independently selected from halogen and C₁-C₆ fluoroalkyl, and
   C₃-C₁₀ cycloalkyl.
      86. The compound, tautomer, deuterated derivative, or pharmaceutically acceptable salt according to any one of embodiments 76 to 85, wherein each R^{N} is independently selected from:
   hydrogen,
   C₁-C₈ alkyl optionally substituted with 1-3 groups independently selected from:
      NH₂,
      NHCOMe,
      C₁-C₆ alkoxy,
      —(O)₀₋₁—(C₃-C₁₀ cycloalkyl),
      C₆-C₁₀ aryl, and
      3- to 14-membered heterocyclyl optionally substituted with 1-4 groups independently selected from C₁-C₆ alkyl, and
   C₆-C₁₀ aryl, and
   or two R^{N} on the same nitrogen atom are taken together with the nitrogen to which they are bonded to form a 3- to 10-membered heterocyclyl optionally substituted with 1-3 groups selected from:
   cyano,
   C₁-C₆ alkyl, and
   C₁-C₆ alkoxy.
      87. A compound of Formula IV:

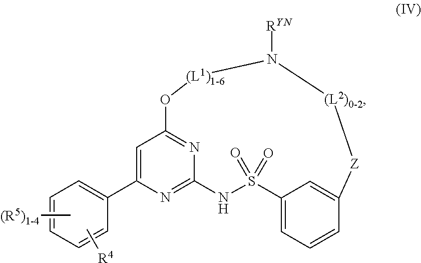

a tautomer thereof, a deuterated derivative of the compound or tautomer, or a pharmaceutically acceptable salt of any of the foregoing, wherein Z, L¹, L², R⁴, R⁵, and R^{YN} are defined as according to embodiment 1, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.
   88. The compound, tautomer, deuterated derivative, or pharmaceutically acceptable salt according to embodiment 87, wherein Z is C(R^{ZC}).
   89. The compound, tautomer, deuterated derivative, or pharmaceutically acceptable salt according to embodiment 87 or 88, wherein two R^{ZC} are taken together to form an oxo group.
   90. The compound, tautomer, deuterated derivative, or pharmaceutically acceptable salt according to any one of embodiments 87 to 89, wherein R⁴ is selected from hydrogen and methyl.
   91. The compound, tautomer, deuterated derivative, or pharmaceutically acceptable salt according to any one of embodiments 87 to 90, wherein R⁴ is methyl.
   92. The compound, tautomer, deuterated derivative, or pharmaceutically acceptable salt according to any one of embodiments 87 to 91, wherein each R⁵ is independently selected from hydrogen, halogen, C₁-C₆ alkyl, C₁-C₆ fluoroalkyl, and C₆-C₁₀ aryl.
   93. The compound, tautomer, deuterated derivative, or pharmaceutically acceptable salt according to any one of embodiments 87 to 92, wherein R^{YN} is selected from:
   C₃-C₁₀ cycloalkyl optionally substituted with 1-3 groups independently selected from:
      hydroxyl,
      cyano,
      N(R^{N})₂,
      C₁-C₆ alkyl optionally substituted with 1-3 groups independently selected from:
         hydroxyl,
         oxo,
         N(R^{N})₂, and
         C₆-C₁₀ aryl,
      C₁-C₆ alkoxy optionally substituted with 1-3 groups independently selected from halogen, oxo, C₆-C₁₀ aryl, and N(R^{N})₂,
      C₃-C₁₀ cycloalkyl, and
      5- to 10-membered heteroaryl optionally substituted with 1-3 groups independently selected from:
         hydroxyl,
         oxo,
         N(R^{N})₂,
         C₁-C₆ alkyl optionally substituted with 1-3 groups independently selected from C₁-C₆ alkoxy, and
         C₁-C₆ alkoxy optionally substituted with 1-3 groups independently selected from C₃-C₁₀ cycloalkyl,
   3- to 10-membered heterocyclyl optionally substituted with 1-3 groups independently selected from:
      C₁-C₆ alkyl optionally substituted with 1-3 groups independently selected from:
         oxo,
         hydroxyl,
         N(R^{N})₂,
         C₁-C₆ alkoxy optionally substituted with 1-3 groups independently selected from C₆-C₁₀ aryl, and
         —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl, and
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl).
         94. The compound, tautomer, deuterated derivative, or pharmaceutically acceptable salt according to any one of embodiments 87 to 93, wherein each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl, and
      C₃-C₁₀ cycloalkyl optionally substituted with 1-3 groups independently selected from halogen and C₁-C₆ fluoroalkyl, and
   C₃-C₁₀ cycloalkyl.
      95. The compound, tautomer, deuterated derivative, or pharmaceutically acceptable salt according to any one of embodiments 87 to 94, wherein each R^{N} is independently selected from:
   hydrogen,
   C₁-C₈ alkyl optionally substituted with 1-3 groups independently selected from:
      NH₂,
      NHCOMe,
      C₁-C₆ alkoxy,
      —(O)₀₋₁—(C₃-C₁₀ cycloalkyl),
      C₆-C₁₀ aryl, and
      3- to 14-membered heterocyclyl optionally substituted with 1-4 groups independently selected from C₁-C₆ alkyl,
   C₆-C₁₀ aryl, and
   or two R^{N} on the same nitrogen atom are taken together with the nitrogen to which they are bonded to form a 3- to 10-membered heterocyclyl optionally substituted with 1-3 groups selected from:
   cyano,
   C₁-C₆ alkyl, and
   C₁-C₆ alkoxy.
      96. A compound of Formula V:

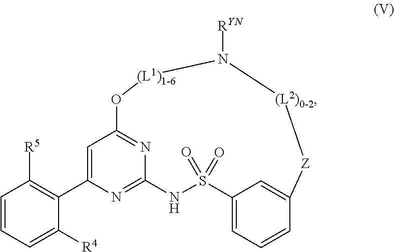

a tautomer thereof, a deuterated derivative of the compound or tautomer, or a pharmaceutically acceptable salt of any of the foregoing, wherein Z, L¹, L², R⁴, R⁵, and R^{YN} are defined as according to embodiment 1, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.
   97. The compound, tautomer, deuterated derivative, or pharmaceutically acceptable salt according to embodiment 96, wherein Z is C(R^{ZC}).
   98. The compound, tautomer, deuterated derivative, or pharmaceutically acceptable salt according to embodiment 96 or 97, wherein two R^{ZC} are taken together to form an oxo group.
   99. The compound, tautomer, deuterated derivative, or pharmaceutically acceptable salt according to any one of embodiments 96 to 98, wherein R⁴ is selected from hydrogen and methyl.
   100. The compound, tautomer, deuterated derivative, or pharmaceutically acceptable salt according to any one of embodiments 96 to 99, wherein R⁴ is methyl.
   101. The compound, tautomer, deuterated derivative, or pharmaceutically acceptable salt according to any one of embodiments 96 to 100, wherein each R⁵ is independently selected from hydrogen, halogen, C₁-C₆ alkyl, C₁-C₆ fluoroalkyl, and C₆-C₁₀ aryl.
   102. The compound, tautomer, deuterated derivative, or pharmaceutically acceptable salt according to any one of embodiments 96 to 101, wherein R^{YN} is selected from:
   C₃-C₁₀ cycloalkyl optionally substituted with 1-3 groups independently selected from:
      hydroxyl,
      cyano,
      N(R^{N})₂,
      C₁-C₆ alkyl optionally substituted with 1-3 groups independently selected from:
         hydroxyl,
         oxo,
         N(R^{N})₂, and
         C₆-C₁₀ aryl,
      C₁-C₆ alkoxy optionally substituted with 1-3 groups independently selected from halogen, oxo, C₆-C₁₀ aryl, and N(R^{N})₂,
      C₃-C₁₀ cycloalkyl,
      5- to 10-membered heteroaryl optionally substituted with 1-3 groups independently selected from:
         hydroxyl,
         oxo,
         N(R^{N})₂,
         C₁-C₆ alkyl optionally substituted with 1-3 groups independently selected from C₁-C₆ alkoxy, and
         C₁-C₆ alkoxy optionally substituted with 1-3 groups independently selected from C₃-C₁₀ cycloalkyl,
   3- to 10-membered heterocyclyl optionally substituted with 1-3 groups independently selected from:
      C₁-C₆ alkyl optionally substituted with 1-3 groups independently selected from:
         oxo,
         hydroxyl,
         N(R^{N})₂,
         C₁-C₆ alkoxy optionally substituted with 1-3 groups independently selected from C₆-C₁₀ aryl, and
         —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl, and
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl).
         103. The compound, tautomer, deuterated derivative, or pharmaceutically acceptable salt according to any one of embodiments 96 to 102, wherein each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl, and
      C₃-C₁₀ cycloalkyl optionally substituted with 1-3 groups independently selected from halogen and C₁-C₆ fluoroalkyl, and
   C₃-C₁₀ cycloalkyl.
      104. The compound, tautomer, deuterated derivative, or pharmaceutically acceptable salt according to any one of embodiments 96 to 103, wherein each R^{N} is independently selected from:
   hydrogen,
   C₁-C₈ alkyl optionally substituted with 1-3 groups independently selected from:
      NH₂,
      NHCOMe,
      C₁-C₆ alkoxy,
      —(O)₀₋₁—(C₃-C₁₀ cycloalkyl),
      C₆-C₁₀ aryl, and
      3- to 14-membered heterocyclyl optionally substituted with 1-4 groups independently selected from C₁-C₆ alkyl, and
   C₆-C₁₀ aryl,
   or two R^{N} on the same nitrogen atom are taken together with the nitrogen to which they are bonded to form a 3- to 10-membered heterocyclyl optionally substituted with 1-3 groups selected from:
   cyano,
   C₁-C₆ alkyl, and
   C₁-C₆ alkoxy.
      105. A compound of Formula VI:

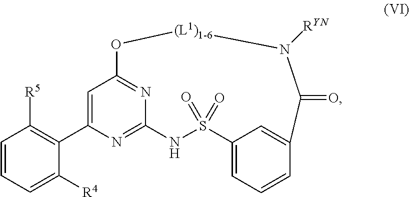

a tautomer thereof, a deuterated derivative of the compound or tautomer, or a pharmaceutically acceptable salt of any of the foregoing, wherein L¹, R⁴, R⁵, and R^{YN} are defined as according to embodiment 1, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.
   106. The compound, tautomer, deuterated derivative, or pharmaceutically acceptable salt according to embodiment 105, wherein R⁴ is selected from hydrogen and methyl.
   107. The compound, tautomer, deuterated derivative, or pharmaceutically acceptable salt according to embodiment 105 or 106, wherein R⁴ is methyl.
   108. The compound, tautomer, deuterated derivative, or pharmaceutically acceptable salt according to any one of embodiments 105 to 107, wherein each R⁵ is independently selected from hydrogen, halogen, C₁-C₆ alkyl, C₁-C₆ fluoroalkyl, and C₆-C₁₀ aryl.
   109. The compound, tautomer, deuterated derivative, or pharmaceutically acceptable salt according to any one of embodiments 105 to 108, wherein R^{YN} is selected from:
   C₃-C₁₀ cycloalkyl optionally substituted with 1-3 groups independently selected from:
      hydroxyl,
      cyano,
      N(R^{N})₂,
      C₁-C₆ alkyl optionally substituted with 1-3 groups independently selected from:
         hydroxyl,
         oxo,
         N(R^{N})₂, and
         C₆-C₁₀ aryl,
      C₁-C₆ alkoxy optionally substituted with 1-3 groups independently selected from halogen, oxo, C₆-C₁₀ aryl, and N(R^{N})₂,
      C₃-C₁₀ cycloalkyl,
      5- to 10-membered heteroaryl optionally substituted with 1-3 groups independently selected from:
         hydroxyl,
         oxo,
         N(R^{N})₂,
         C₁-C₆ alkyl optionally substituted with 1-3 groups independently selected from C₁-C₆ alkoxy, and
         C₁-C₆ alkoxy optionally substituted with 1-3 groups independently selected from C₃-C₁₀ cycloalkyl,
   3- to 10-membered heterocyclyl optionally substituted with 1-3 groups independently selected from:
      C₁-C₆ alkyl optionally substituted with 1-3 groups independently selected from:
         oxo,
         hydroxyl,
         N(R^{N})₂,
         C₁-C₆ alkoxy optionally substituted with 1-3 groups independently selected from C₆-C₁₀ aryl, and
         —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl, and
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl).
         110. The compound, tautomer, deuterated derivative, or pharmaceutically acceptable salt according to any one of embodiments 105 to 109, wherein each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl, and
      C₃-C₁₀ cycloalkyl optionally substituted with 1-3 groups independently selected from halogen and C₁-C₆ fluoroalkyl, and
   C₃-C₁₀ cycloalkyl.
      111. The compound, tautomer, deuterated derivative, or pharmaceutically acceptable salt according to any one of embodiments 105 to 110, wherein each R^{N} is independently selected from:
   hydrogen,
   C₁-C₈ alkyl optionally substituted with 1-3 groups independently selected from:
      NH₂,
      NHCOMe,
      C₁-C₆ alkoxy,
      —(O)₀₋₁—(C₃-C₁₀ cycloalkyl),
      C₆-C₁₀ aryl, and
      3- to 14-membered heterocyclyl optionally substituted with 1-4 groups independently selected from C₁-C₆ alkyl, and
   C₆-C₁₀ aryl,
   or two R^{N} on the same nitrogen atom are taken together with the nitrogen to which they are bonded to form a 3- to 10-membered heterocyclyl optionally substituted with 1-3 groups selected from:
   cyano,
   C₁-C₆ alkyl, and
   C₁-C₆ alkoxy.
      112. The compound, tautomer, deuterated derivative, or pharmaceutically acceptable salt according to any one of embodiments 1 to 111, selected from compounds of Formulae I, Ia, IIa, IIb, III, IV, V, and VI, tautomers thereof, deuterated derivatives of those compounds and tautomers, and pharmaceutically acceptable salts of any of the foregoing.
      113. The compound, tautomer, deuterated derivative, or pharmaceutically acceptable salt according to any one of embodiments 1 to 112, selected from Compounds 1-474 (Tables 8, 9, 10, 11), Compounds 475-506 (Table 7), Compounds 507 and 508 (Table 12), tautomers thereof, deuterated derivatives of those compounds and tautomers, and pharmaceutically acceptable salts of any of the foregoing.
      114. A pharmaceutical composition comprising the compound, tautomer, deuterated derivative, or pharmaceutically acceptable salt according to any one of embodiments 1 to 113, and a pharmaceutically acceptable carrier.
      115. The pharmaceutical composition of embodiment 114, further comprising one or more additional therapeutic agents.
      116. The pharmaceutical composition of embodiment 115, wherein the one or more additional therapeutic agents is selected from mucolytic agents, bronchodilators, antibiotics, anti-infective agents, and anti-inflammatory agents.
      117. The pharmaceutical composition of embodiment 115, wherein the one or more additional therapeutic agent is an antibiotic selected from tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.
      118. The pharmaceutical composition of embodiment 115, wherein the one or more additional therapeutic agent is one or more CFTR modulating agents.
      119. The pharmaceutical composition of embodiment 118, wherein the one or more CFTR modulating agents are selected from CFTR potentiators.
      120. The pharmaceutical composition of embodiment 118, wherein the one or more CFTR modulating agents are selected from CFTR correctors.
      121. The pharmaceutical composition of embodiment 118, wherein the one or more CFTR modulating agents comprises at least one CFTR potentiator and at least one CFTR corrector.
      122. The pharmaceutical composition of any one of embodiments 118 to 121, wherein the one or more CFTR modulating agents are selected from (a) tezacaftor, lumacaftor, and deuterated derivatives and pharmaceutically acceptable salts thereof; and (b) ivacaftor, deutivacaftor, and deuterated derivatives and pharmaceutically acceptable salts of any of the foregoing.
      123. The pharmaceutical composition of any one of embodiments 118 to 121, wherein the one or more CFTR modulating agents are selected from (a) tezacaftor, lumacaftor, and deuterated derivatives and pharmaceutically acceptable salts thereof; or (b) (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol and deuterated derivatives and pharmaceutically acceptable salts thereof.
      124. The pharmaceutical composition of any one of embodiments 118 to 121, wherein the composition comprises tezacaftor and ivacaftor.
      125. The pharmaceutical composition of any one of embodiments 118 to 121, wherein the composition comprises tezacaftor and deutivacaftor.
      126. The pharmaceutical composition of any one of embodiments 118 to 121, wherein the composition comprises tezacaftor and (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol.
      127. The pharmaceutical composition of any one of embodiments 118 to 121, wherein the composition comprises lumacaftor and ivacaftor.
      128. The pharmaceutical composition of any one of embodiments 118 to 121, wherein the composition comprises lumacaftor and deutivacaftor.
      129. The pharmaceutical composition of any one of embodiments 118 to 121, wherein the composition comprises lumacaftor and (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol.
      130. A method of treating cystic fibrosis comprising administering to a patient in need thereof the compound, tautomer, deuterated derivative, or pharmaceutically acceptable salt according to any one of embodiments 1 to 113, or a pharmaceutical composition according to any one of embodiments 114 to 129.
      131. The method of embodiment 130, further comprising administering to the patient one or more additional therapeutic agents prior to, concurrent with, or subsequent to the compound, tautomer, deuterated derivative, or pharmaceutically acceptable salt according to any one of embodiments 1 to 113, or the pharmaceutical composition according to embodiment 114.
      132. The method of embodiment 131, wherein the one or more additional therapeutic agents is(are) selected from CFTR modulating agents.
      133. The method of embodiment 132, wherein the one or more CFTR modulating agents are selected from CFTR potentiators.
      134. The method of embodiment 132, wherein the one or more CFTR modulating agents are selected from CFTR correctors.
      135. The method of embodiment 132, wherein the one or more CFTR modulating agents comprise both a CFTR potentiator and an additional CFTR corrector.
      136. The method of embodiment 133 and 135, wherein the CFTR potentiator is selected from ivacaftor, deutivacaftor, (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol, and deuterated derivatives and pharmaceutically acceptable salts of any of the foregoing.
      137. The method of embodiment 134 or embodiment 135, wherein the CFTR corrector is selected from tezacaftor, lumacaftor, and deuterated derivatives and pharmaceutically acceptable salts thereof.
      138. The method of embodiment 131, wherein the one or more additional therapeutic agent(s) is a compound selected from tezacaftor, ivacaftor, deutivacaftor, lumacaftor, and pharmaceutically acceptable salts thereof.
      139. The method of embodiment 131, wherein the one or more additional therapeutic agent(s) is a compound selected from (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol and deuterated derivatives and pharmaceutically acceptable salts thereof.
      140. The method of embodiment 131, wherein the one or more additional therapeutic agents are tezacaftor and ivacaftor.
      141. The method of embodiment 131, wherein the one or more additional therapeutic agents are tezacaftor and deutivacaftor.
      142. The method of embodiment 131, wherein the one or more additional therapeutic agents are tezacaftor and (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol.
      143. The method of embodiment 131, wherein the one or more additional therapeutic agents are lumacaftor and ivacaftor.
      144. The method of embodiment 131, wherein the one or more additional therapeutic agents are lumacaftor and deutivacaftor.
      145. The method of embodiment 131, wherein the one or more additional therapeutic agents are lumacaftor and (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol.
      146. The compound, tautomer, deuterated derivative, or pharmaceutically acceptable salt according to any one of embodiments 1 to 113, or the pharmaceutical composition according to any one of embodiments 114 to 129 for use in the treatment of cystic fibrosis.
      147. The compound, tautomer, deuterated derivative, or pharmaceutically acceptable salt according to any one of embodiments 1 to 113, or the pharmaceutical composition according to any one of embodiments 114 to 129 for use in the manufacture of a medicament for the treatment of cystic fibrosis.
      148. A compound selected from Compounds 1-508, tautomers thereof, deuterated derivatives of those compounds and tautomers, and pharmaceutically acceptable salts of any of the foregoing.
      149. A deuterated derivative of a compound selected from Compounds 1-508.
      150. A pharmaceutically acceptable salt of a compound selected from Compounds 1-508.
      151. A compound selected from Compounds 1-508.
      152. A pharmaceutical composition comprising a compound selected from Compounds 1-508, tautomers thereof, deuterated derivatives of those compounds and tautomers, and pharmaceutically acceptable salts of any of the foregoing and a pharmaceutically acceptable carrier.
      153. A pharmaceutical composition comprising a deuterated derivative of a compound selected from Compounds 1-508 and a pharmaceutically acceptable carrier.
      154. A pharmaceutical composition comprising a pharmaceutically acceptable salt of a compound selected from Compounds 1-508 and a pharmaceutically acceptable carrier.
      155. A pharmaceutical composition comprising a compound selected from Compounds 1-508 and a pharmaceutically acceptable carrier.
      156. A pharmaceutical composition comprising (a) a compound selected from Compounds 1-508, tautomers thereof, deuterated derivatives of those compounds and tautomers, and pharmaceutically acceptable salts of any of the foregoing; (b) a CFTR potentiator; and (c) a pharmaceutically acceptable carrier.
      157. A pharmaceutical composition comprising (a) a deuterated derivative of a compound selected from Compounds 1-508; (b) a CFTR potentiator; and (c) a pharmaceutically acceptable carrier.
      158. A pharmaceutical comprising (a) a pharmaceutically acceptable salt of a compound selected from Compounds 1-508; (b) a CFTR potentiator; and (c) a pharmaceutically acceptable carrier.
      159. A pharmaceutical composition comprising (a) a compound selected from Compounds 1-508; (b) a CFTR potentiator; and (c) a pharmaceutically acceptable carrier.
      160. A pharmaceutical composition comprising (a) a compound selected from Compounds 1-508, tautomers thereof, deuterated derivatives of those compounds and tautomers, and pharmaceutically acceptable salts of any of the foregoing; (b) an additional CFTR corrector; and (c) a pharmaceutically acceptable carrier.
      161. A pharmaceutical composition comprising (a) a deuterated derivative of a compound selected from Compounds 1-508; (b) an additional CFTR corrector; and (c) a pharmaceutically acceptable carrier.
      162. A pharmaceutical composition comprising (a) a pharmaceutically acceptable salt of a compound selected from Compounds 1-508; (b) an additional CFTR corrector; and (c) a pharmaceutically acceptable carrier.
      163. A pharmaceutical composition comprising (a) a compound selected from Compounds 1-508; (b) an additional CFTR corrector; and (c) a pharmaceutically acceptable carrier.
      164. A pharmaceutical composition comprising (a) a compound selected from Compounds 1-508, tautomers thereof, deuterated derivatives of those compounds and tautomers, and pharmaceutically acceptable salts of any of the foregoing; (b) an additional CFTR corrector; (c) a CRTR potentiator; and (d) a pharmaceutically acceptable carrier.
      165. A pharmaceutical composition comprising (a) a deuterated derivative of a compound selected from Compounds 1-508; (b) an additional CFTR corrector; (c) a CFTR potentiator; and (d) a pharmaceutically acceptable carrier.
      166. A pharmaceutical composition comprising (a) a pharmaceutically acceptable salt of a compound selected from Compounds 1-508; (b) an additional CFTR corrector; (c) a CFTR potentiator; and (d) a pharmaceutically acceptable carrier.
      167. A pharmaceutical composition comprising (a) a compound selected from Compounds 1-508; (b) an additional CFTR corrector; (c) a CFTR potentiator; and (d) a pharmaceutically acceptable carrier.
      168. A compound selected from Compounds 1-508, tautomers thereof, deuterated derivatives of those compounds and tautomers, and pharmaceutically acceptable salts of any of the foregoing for use in a method of treating cystic fibrosis.
      169. A deuterated derivative of a compound selected from Compounds 1-508 for use in a method of treating cystic fibrosis.
      170. A pharmaceutically acceptable salt of a compound selected from Compounds 1-508 for use in a method of treating cystic fibrosis.
      171. A compound selected from Compounds 1-508 for use in a method of treating cystic fibrosis.
      172. A pharmaceutical composition comprising a compound selected from Compounds 1-508, tautomers thereof, deuterated derivatives of those compounds and tautomers, and pharmaceutically acceptable salts of any of the foregoing and a pharmaceutically acceptable carrier for use in a method of treating cystic fibrosis.
      173. A pharmaceutical composition comprising a deuterated derivative of a compound selected from Compounds 1-508 and a pharmaceutically acceptable carrier for use in a method of treating cystic fibrosis.
      174. A pharmaceutical composition comprising a pharmaceutically acceptable salt of a compound selected from Compounds 1-508 and a pharmaceutically acceptable carrier for use in a method of treating cystic fibrosis.
      175. A pharmaceutical composition comprising a compound selected from Compounds 1-508 and a pharmaceutically acceptable carrier for use in a method of treating cystic fibrosis.
      176. A pharmaceutical composition comprising (a) a compound selected from Compounds 1-508, tautomers thereof, deuterated derivatives of those compounds and tautomers, and pharmaceutically acceptable salts of any of the foregoing; (b) a CFTR potentiator; and (c) a pharmaceutically acceptable carrier for use in a method of treating cystic fibrosis.
      177. A pharmaceutical comprising (a) a deuterated derivative of a compound selected from Compounds 1-508; (b) a CFTR potentiator; and (c) a pharmaceutically acceptable carrier for use in a method of treating cystic fibrosis.
      178. A pharmaceutical composition comprising (a) a pharmaceutically acceptable salt of a compound selected from Compounds 1-508; (b) a CFTR potentiator; and (c) a pharmaceutically acceptable carrier for use in a method of treating cystic fibrosis.
      179. A pharmaceutical composition comprising (a) a compound selected from Compounds 1-508; (b) a CFTR potentiator; and (c) a pharmaceutically acceptable carrier.
      180. A pharmaceutical composition comprising (a) a compound selected from Compounds 1-508, tautomers thereof, deuterated derivatives of those compounds and tautomers, and pharmaceutically acceptable salts of any of the foregoing; (b) an additional CFTR corrector; and (c) a pharmaceutically acceptable carrier for use in a method of treating cystic fibrosis.
      181. A pharmaceutical composition comprising (a) a deuterated derivative of a compound selected from Compounds 1-508; (b) an additional CFTR corrector; and (c) a pharmaceutically acceptable carrier for use in a method of treating cystic fibrosis.
      182. A pharmaceutical composition comprising (a) a pharmaceutically acceptable salt of a compound selected from Compounds 1-508; (b) an additional CFTR corrector; and (c) a pharmaceutically acceptable carrier for use in a method of treating cystic fibrosis.
      183. A pharmaceutical composition comprising (a) a compound selected from Compounds 1-508; (b) an additional CFTR corrector; and (c) a pharmaceutically acceptable carrier for use in a method of treating cystic fibrosis.
      184. A pharmaceutical composition comprising (a) a compound selected from Compounds 1-508, tautomers thereof, deuterated derivatives of those compounds and tautomers, and pharmaceutically acceptable salts of any of the foregoing; (b) an additional CFTR corrector; (c) a CRTR potentiator; and (d) a pharmaceutically acceptable carrier for use in a method of treating cystic fibrosis.
      185. A pharmaceutical composition comprising (a) a deuterated derivative of a compound selected from Compounds 1-508; (b) an additional CFTR corrector; (c) a CFTR potentiator; and (d) a pharmaceutically acceptable carrier for use in a method of treating cystic fibrosis.
      186. A pharmaceutical composition comprising (a) a pharmaceutically acceptable salt of a compound selected from Compounds 1-508; (b) an additional CFTR corrector; (c) a CFTR potentiator; and (d) a pharmaceutically acceptable carrier for use in a method of treating cystic fibrosis.
      187. A pharmaceutical composition comprising (a) a compound selected from Compounds 1-508; (b) an additional CFTR corrector; (c) a CFTR potentiator; and (d) a pharmaceutically acceptable carrier for use in a method of treating cystic fibrosis.

### EXAMPLES

### I. Abbreviation List

ACN: Acetonitrile
Boc anhydride ((Boc)₂O): Di-tert-butyl dicarbonate
CDCl₃: Chloroform-d
CDI: Carbonyl diimidazole
CDMT: 2-Chloro-4,6-dimethoxy-1,3,5-triazine
CH₂Cl₂: Dichloromethane
CH₃CN: Acetonitrile
COMU: (1-Cyano-2-ethoxy-2-oxoethylidenaminooxy)dimethylamino-morpholino-carbenium hexafluorophosphate
Cmpd: Compound
DABCO: 1,4-Diazabicyclo[2.2.2]octane
DBU: 1,8-Diazabicyclo(5.4.0)undec-7-ene
DCE: 1,2-Dichloroethane
DCM: Dichloromethane
DI: Deionized
DIAD: Diisopropyl azodicarboxylate
DIEA: (DIPEA, DiPEA): N,N-diisopropylethylamine
DMA: N,N-Dimethylacetamide
DMAP: 4-Dimethylaminopyridine
DMF: N,N-Dimethylformamide
DMSO: Dimethyl sulfoxide
DMP: Dess-Martin periodinane
EA: Ethyl acetate
EDC: 1-Ethyl-3-(3-dimethylaminopropyl)carbodiimide
ELSD: Evaporative light scattering detector
diethylether: Diethyl ether
ESI-MS: Electrospray ionization mass spectrometry
EtOAc: Ethyl acetate
EtOH: Ethanol
GC: Gas chromatography
Grubbs 1^{st} Generation catalyst: Dichloro(benzylidene)bis(tricyclohexylphosphine)ruthenium(II)
Grubbs 2^{nd} Generation catalyst: [1,3-Bis(2,4,6-trimethylphenyl)imidazolidin-2-ylidene]-dichloro-[(2-isopropoxyphenyl)methylene]ruthenium
HATU: 1-[Bis(dimethylamino)methylene]-1H-1,2,3-triazolo[4,5-b]pyridinium 3-oxid hexafluorophosphate
HPLC: High-performance liquid chromatography
Hoveyda-Grubbs 2^{nd} Generation catalyst: (1,3-Bis-(2,4,6-trimethylphenyl)-2-imidazolidinylidene)dichloro(o-isopropoxyphenylmethylene)ruthenium, Dichloro[1,3-bis(2,4,6-trimethylphenyl)-2-imidazolidinylidene](2-isopropoxyphenylmethylene)ruthenium(II)
IPA: Isopropanol
KHSO₄: Potassium bisulfate
LC: Liquid chromatography
LCMS: Liquid chromatography mass spectrometry
LCMS Met.: LCMS method
LCMS Rt: LCMS retention time
LDA: Lithium diisopropylamide
LiOH: Lithium hydroxide
MeCN: Acetonitrile
MeOH: Methanol
MTBE: Methyl tert-butyl ether
MeTHF or 2-MeTHF: 2-Methyltetrahydrofuran
MgSO₄: Magnesium sulfate
NaHCO₃: Sodium bicarbonate
NaOH: Sodium hydroxide
NMP: N-Methyl-2-pyrrolidone
NMM: N-Methylmorpholine
Pd₂(dba)₃: Tris(dibenzylideneacetone)dipalladium(0)
Pd/C: Palladium on carbon
Pd(dppf)Cl₂: [1,1′-Bis(diphenylphosphino)ferrocene]dichloropalladium(II)
Pd(OAc)₂: Palladium(II) acetate
PTFE: Polytetrafluoroethylene
rt, RT: Room temperature
RuPhos: 2-Dicyclohexylphosphino-2′,6′-diisopropoxybiphenyl
SFC: Supercritical fluid chromatography
TBAI: Tetrabutylammonium iodide
TEA: Triethylamine
TFA: Trifluoroacetic acid
THF: Tetrahydrofuran
TLC: Thin layer chromatography
TMS: Trimethylsilyl
TMSCl: Trimethylsilyl chloride
T3P: Propanephosphonic acid anhydride
UPLC: Ultra Performance Liquid Chromatography
XANTPHOS: 4,5-Bis(diphenylphosphino)-9,9-dimethylxanthene
XPhos: 2-Dicyclohexylphosphino-2′,4′,6′-triisopropylbiphenyl

### II. General Methods

Reagents and starting materials were obtained by commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz and 28860 points of acquisition. FID were zero-filled to 32 k points and a line broadening of 0.3 Hz was applied before Fourier transform. ¹⁹F NMR spectra were recorded at 282 MHz using a 30 degree pulse angle, a spectral width of 100 kHz and 59202 points were acquired. FID were zero-filled to 64 k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz and 128 k points of acquisition. FID were zero-filled to 256 k points and a line broadening of 0.3 Hz was applied before Fourier transform. 19F NMR spectra were recorded at 377 MHz using a 30 deg pulse angle, a spectral width of 89286 Hz and 128 k points were acquired. FID were zero-filled to 256 k points and a line broadening of 0.3 Hz was applied before Fourier transform.

NMR spectra were also recorded on a Bruker AC 250 MHz instrument equipped with a: 5 mm QNP(H1/C13/F19/P31) probe (type: 250-SB, s #23055/0020) or on a Varian 500 MHz instrument equipped with a ID PFG, 5 mm, 50-202/500 MHz probe (model/part #99337300).

Final purity of compounds was determined by reversed phase UPLC using an Acquity UPLC BEH C₁₈ column (50×2.1 mm, 1.7 m particle) made by Waters (pn: 186002350), and a dual gradient run from 1-99% mobile phase B over 3.0 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+1] species obtained using a single quadrupole mass spectrometer equipped with an electrospray ionization (ESI) source capable of achieving a mass accuracy of 0.1 Da and a minimum resolution of 1000 (no units on resolution) across the detection range. Optical purity of methyl (2S)-2,4-dimethyl-4-nitro-pentanoate was determined using chiral gas chromatography (GC) analysis on an Agilent 7890A/MSD 5975C instrument, using a Restek Rt-βDEXcst (30 m×0.25 mm×0.25 μm_df) column, with a 2.0 mL/min flow rate (H₂ carrier gas), at an injection temperature of 220° C. and an oven temperature of 120° C., 15 minutes.

### III. General UPLC/HPLC Analytical Methods

LC method A: Analytical reverse phase UPLC using an Acquity UPLC BEH C18 column (50×2.1 mm, 1.7 m particle) made by Waters (pn: 186002350), and a dual gradient run from 1-99% mobile phase B over 3.0 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

LC method D: Acquity UPLC BEH C₁₈ column (30×2.1 mm, 1.7 m particle) made by Waters (pn: 186002349), and a dual gradient run from 1-99% mobile phase B over 1.0 minute. Mobile phase A=H₂O (0.05% CF₃CO₂H). Mobile phase B═CH₃CN (0.035% CF₃CO₂H). Flow rate=1.5 mL/min, injection volume=1.5 μL, and column temperature=60° C.

LC method I: Acquity UPLC BEH C₁₈ column (50×2.1 mm, 1.7 m particle) made by Waters (pn:186002350), and a dual gradient run from 1-99% mobile phase B over 5.0 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

LC method J: Reverse phase UPLC using an Acquity UPLC BEH C₁₈ column (50×2.1 mm, 1.7 m particle) made by Waters (pn: 186002350), and a dual gradient run from 1-99% mobile phase B over 2.9 minutes. Mobile phase A=H₂O (0.05% NH₄HCO₂). Mobile phase B═CH₃CN. Flow rate=1.2 mL/min, injection volume=1.5 μL, and column temperature=60° C.

LC method K: Kinetex Polar C₁₈ 3.0×50 mm 2.6 μm, 3 min, 5-95% ACN in H₂O (0.1% Formic Acid) 1.2 mL/min.

LC method Q: Reversed phase UPLC using an Acquity UPLC BEH C₁₈ column (50×2.1 mm, 1.7 m particle) made by Waters (pn: 186002350), and a dual gradient run from 30-99% mobile phase B over 2.9 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

LC method S: Merckmillipore Chromolith SpeedROD C₁₈ column (50×4.6 mm) and a dual gradient run from 5-100% mobile phase B over 12 minutes. Mobile phase A=water (0.1% CF₃CO₂H). Mobile phase B=acetonitrile (0.1% CF₃CO₂H).

LC method T: Merckmillipore Chromolith SpeedROD C₁₈ column (50×4.6 mm) and a dual gradient run from 5-100% mobile phase B over 6 minutes. Mobile phase A=water (0.1% CF₃CO₂H). Mobile phase B=acetonitrile (0.1% CF₃CO₂H).

LC method U: Kinetex Polar C₁₈ 3.0×50 mm 2.6 μm, 6 min, 5-95% ACN in H₂O (0.1% Formic Acid) 1.2 mL/min.

LC method V: Acquity UPLC BEH C₁₈ column (50×2.1 mm, 1.7 m particle) made by Waters (pn: 186002350), and a dual gradient run from 1-30% mobile phase B over 2.9 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

LC method W: water Cortex 2.7μ C₁₈ (3.0 mm×50 mm), Temp: 55° C.; Flow: 1.2 mL/min; mobile phase: 100% water with 0.1% trifluoroacetic (TFA) acid then 100% acetonitrile with 0.1% TFA acid, grad:5% to 100% B over 4 min, with stay at 100% B for 0.5 min, equilibration to 5% B over 1.5 min.

LC method X: UPLC Luna C₁₈(2) 50×3 mm 3 μm. run: 2.5 min. Mobile phase: Initial 95% H₂O 0.1% FA/5% MeCN 0.1% FA, linear grad to 95% MeCN 0.1% FA over 1.3 min, hold 1.2 min 95% CH₃CN 0.1% FA, T: 45 C, Flow: 1.5 mL/min

LC method Y: UPLC SunFire C₁₈ 75×4.6 mm 3.5 μm, run: 6 min. Mobile phase conditions: Initial 95% H₂O+0.1% FA/5% CH₃CN+0.1% FA, linear gradient to 95% CH₃CN for 4 min, hold for 2 min at 95% CH₃CN. T:45° C., Flow: 1.5 mL/min

LC method 1A: Reversed phase UPC2 using a Viridis BEH 2-Ethylpyridine column (150×2.1 mm, 3.5 μm particle) made by Waters (pn: 186006655), and a dual gradient run from 5-80% mobile phase B over 4.5 minutes. Mobile phase A=CO₂. Mobile phase B=MeOH (20 mM NH₃). Variable flow rate=1.30-0.40 mL/min to maintain constant pressure, injection volume=2.0 μL, and column temperature=55° C.

### IV. Synthesis of Common Intermediates

### Example A: Preparation of 3-[[4-Chloro-6-(2,6-dimethylphenyl)pyrimidin-2-yl]sulfamoyl]benzoic acid

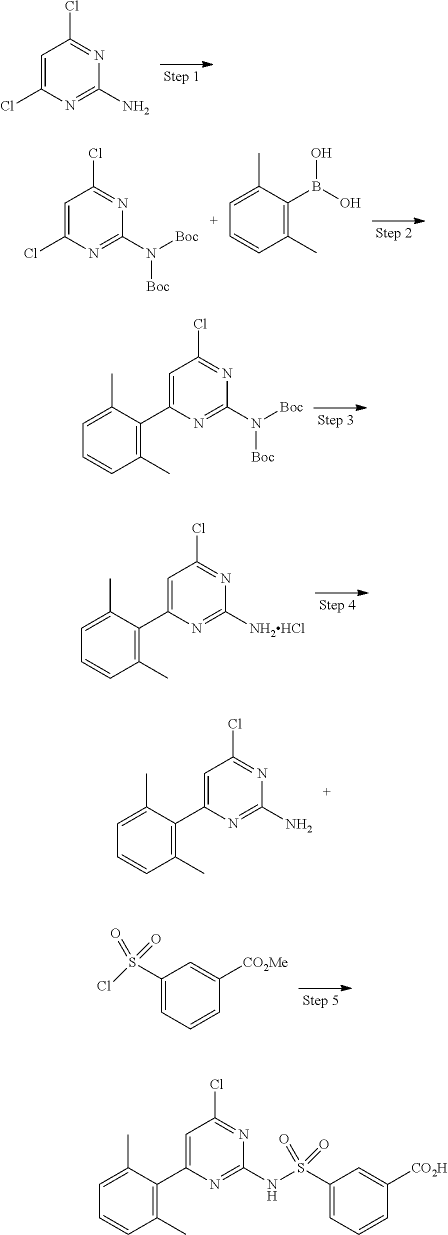 [1218] Step 1: tert-Butyl N-tert-butoxycarbanyl-N-(4,6-dichloropyrimidin-2-yl)carbamate

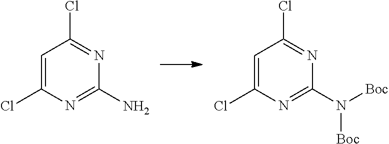 [SMILES: Nc1nc(Cl)cc(Cl)n1 ; CC(C)(C)OC(=O)N(C(=O)OC(C)(C)C)c1nc(Cl)cc(Cl)n1]

To a solution of 4,6-dichloropyrimidin-2-amine (300 g, 1.829 mol) in DCM (2.1 L) was added (BOC)₂O (838 g, 3.840 mol) followed by DMAP (5.6 g, 45.84 mmol). The mixture was stirred at ambient temperature for 6 h. Additional DMAP (5.6 g, 45.84 mmol) was added and the reaction was continued to stir at ambient temperature for 24 h. The mixture was diluted with water (2.1 L) and the organic phase separated. The organic phase was washed with water (2.1 L), 2.1 L of brine, dried over magnesium sulfate, filtered over Celite and concentrated in vacuo affording a light orange oil which had a silt in the slurry. The mixture was diluted with ˜500 mL of heptane and filtered using an M filter. The precipitate (SM) was washed with 250 mL of heptane. The filtrate was concentrated in vacuo affording a thick orange oil which was seeded with solid from a previous experiment and crystallized on standing, affording a light orange hard solid. tert-butyl N-tert-butoxycarbonyl-N-(4,6-dichloropyrimidin-2-yl)carbamate (645 g, 97%). ¹H NMR (400 MHz, DMSO-d6) δ 8.07 (s, 1H), 1.44 (s, 18H). ESI-MS m/z calc. 363.07526, found 364.1 (M+1)+; Retention time: 2.12 minutes (LC method A).

### Step 2: tert-Butyl N-tert-butoxycarbonyl-N-[4-chloro-6-(2,6-dimethylphenyl)pyrimidin-2-yl]carbamate

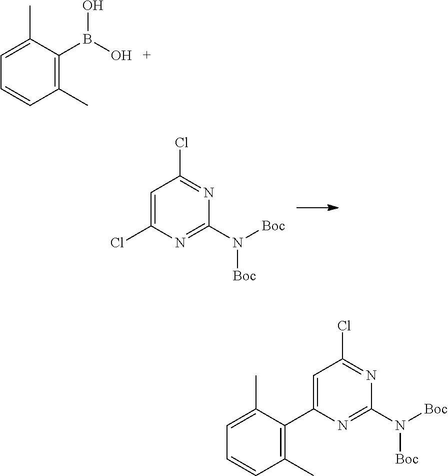

All solvents were degassed prior to use. To a slurry of tert-butyl N-tert-butoxycarbonyl-N-(4,6-dichloropyrimidin-2-yl)carbamate (88 g, 241.6 mmol), (2,6-dimethylphenyl)boronic acid (approximately 36.24 g, 241.6 mmol) and Cs₂CO₃ (approximately 196.8 g, 604.0 mmol) in DME (704 mL) and water (176 mL) were added Pd(dppf)Cl₂ (approximately 8.839 g, 12.08 mmol) was added and the mixture was vigorously stirred under nitrogen at 80° C. (reflux) for 1 h (no SM remained). The reaction was cooled to ambient temperature and diluted with water (704 mL). The aqueous phase was separated and extracted with EtOAc (704 mL). The organic phase was washed with 700 mL of brine, dried over magnesium sulfate, filtered and concentrated in vacuo. The crude product was chromatographed on a 1500 g silica gel column eluting with 0-30% EtOAc/hexanes. The product fractions (eluted at 15% EtOAc) were combined and concentrated in vacuo affording the product as a clear oil which crystallized on standing. tert-butyl N-tert-butoxycarbonyl-N-[4-chloro-6-(2,6-dimethylphenyl)pyrimidin-2-yl]carbamate (81.3 g, 78%). ¹H NMR (400 MHz, DMSO-d₆) δ 7.88 (s, 1H), 7.30 (dd, J=8.2, 7.0 Hz, 1H), 7.21-7.16 (m, 2H), 2.03 (s, 6H), 1.38 (s, 18H). ESI-MS m/z calc. 433.17682, found 434.1 (M+1)+; Retention time: 2.32 minutes (LC method A).

### Step 3: 4-Chloro-6-(2,6-dimethylphenyl)pyrimidin-2-amine (hydrochloride salt)

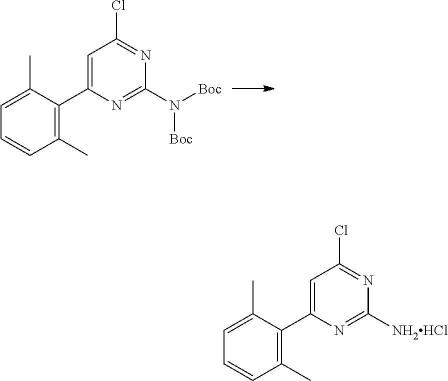

tert-Butyl N-tert-butoxycarbonyl-N-[4-chloro-6-(2,6-dimethylphenyl) pyrimidin-2-yl]carbamate (514.8 g, 915.9 mmol) was dissolved in dichloromethane (4 L). Hydrogen chloride in p-dioxane (1 L, 4 mol) was added and the mixture was stirred overnight at room temperature. The resulting precipitate was collected by vacuum filtration and dried in vacuo to obtain 4-chloro-6-(2,6-dimethylphenyl)pyrimidin-2-amine hydrochloride as a white solid (213.5 g, 82%). ¹H NMR (250 MHz, DMSO-d₆) δ 7.45-6.91 (m, 3H), 6.73 (s, 1H), 2.08 (s, 6H). ESI-MS m/z calc. 233.072, found 234.1 (M+1)+; Retention time: 2.1 minutes (LC Method C).

### Step 4: 4-Chloro-6-(2,6-dimethylphenyl)pyrimidin-2-amine

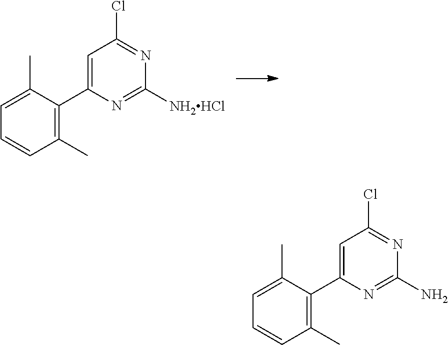

4-Chloro-6-(2,6-dimethylphenyl)pyrimidin-2-amine (hydrochloride salt) (166 g, 614.5 mmol) and 4-chloro-6-(2,6-dimethylphenyl)pyrimidin-2-amine (hydrochloride salt) (30 g, 111.0 mmol) were suspended in DCM (2.5 L), treated with NaOH (725 mL of 1 M, 725.0 mmol) and stirred at room temperature for 1 hour. The mixture was transferred into a separatory funnel and left standing over night. The DCM phase was separated and the aqueous phase with insoluble material was extracted twice more with DCM (2×500 mL). The combined brown DCM phases were stirred over magnesium sulfate and charcoal for 1 hour, filtered and the yellow solution concentrated to a volume of ˜ 500 mL. The solution was diluted with heptane (750 mL) and DCM was removed under reduced pressure at 60° C. to give a cream suspension. It was stirred at room temperature for 1 hour, filtered, washed with cold heptane and dried to give 4-chloro-6-(2,6-dimethylphenyl)pyrimidin-2-amine (157 g, 91%) as a cream solid. ¹H NMR (400 MHz, DMSO-d₆) δ 7.28-7.14 (m, 3H), 7.10 (d, J=7.5 Hz, 2H), 6.63 (s, 1H), 2.06 (s, 6H). ESI-MS m/z calc. 233.07198, found 234.0 (M+1)+; Retention time: 1.45 minutes (LC method A).

### Step 5: 3-[[4-Chloro-6-(2,6-dimethylphenyl)pyrimidin-2-yl]sulfamoyl]benzoic acid

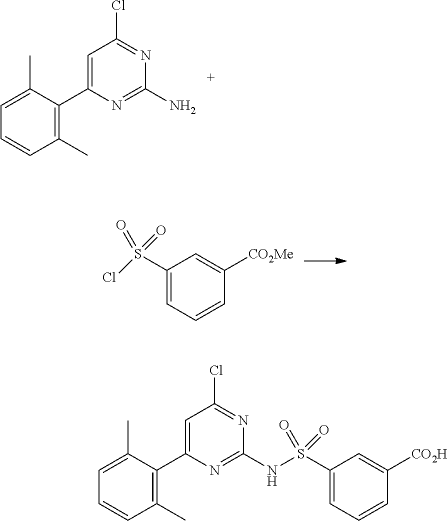

4-Chloro-6-(2,6-dimethylphenyl)pyrimidin-2-amine (235 g, 985.5 mmol) was dissolved in MeTHF (2.3 L) and cooled in an ice bath under stirring and nitrogen. To the cold solution methyl 3-chlorosulfonylbenzoate (347 g, 1.479 mol) was added in one portion (seems slightly endothermic) and to the cold pale-yellow solution a solution of 2-methyl-butan-2-ol (Lithium salt) (875 mL of 3.1 M, 2.712 mol) (in heptane) was added dropwise over 1.25 hour (exothermic, internal temperature from 0 to 10° C.). The ice bath was removed and the greenish solution was stirred for 4 hours at room temperature. To the greenish solution cold HCl (2 L of 1.5 M, 3.000 mol) was added, the phases separated and the organic phase was washed once with water (1 L) and once with brine (500 mL). The aqueous phases were back extracted once with MeTHF (350 mL) and the organic phases were combined. This yellow MeTHF solution of methyl 3-[[4-chloro-6-(2,6-dimethylphenyl)pyrimidin-2-yl]sulfamoyl]benzoate (ESI-MS m/z calc. 431.07065, found 432.0 (M+1)+; Retention time: 1.81 minutes) was treated with NaOH (2.3 L of 2 M, 4.600 mol) and stirred at room temperature for 1 hour. The phases were separated and the NaOH phase was washed twice with MeTHF (2×500 mL) and the combined organic phases were extracted once with 2M NaOH (1×250 mL). The combined NaOH phases were combined, stirred in an ice bath and slowly acidified by addition of HCl (416 mL of 36% w/w, 4.929 mol) while keeping the internal temperature between 10 and 20° C. At the end of the addition (pH˜5-6) the final pH was adjusted to 2-3 by addition of solid citric acid. The formed yellow tacky suspension was stirred at room temperature overnight to give a cream crisp suspension. The solid was collected by filtration, washed with plenty of water and sucked dry for 3 hours. The solid was dried under reduced pressure with a nitrogen leak at 45-50° C. for 120 hours. 3-[[4-chloro-6-(2,6-dimethylphenyl)pyrimidin-2-yl]sulfamoyl]benzoic acid (395 g, 96%) was isolated as an off-white solid. ¹H NMR (400 MHz, DMSO-d₆) δ 13.44 (s, 1H), 12.46 (s, 1H), 8.48-8.39 (m, 1H), 8.25-8.15 (m, 1H), 8.15-8.08 (m, 1H), 7.68 (t, J=7.8 Hz, 1H), 7.31 (s, 1H), 7.28-7.18 (m, 1H), 7.10 (d, J=7.6 Hz, 2H), 1.84 (s, 6H). ESI-MS m/z calc. 417.055, found 418.0 (M+1)+; Retention time: 1.56 minutes. (LC method A).

### Example B: Preparation of 3-[[4-[(2R)-2-(tert-Butoxycarbonylamino)-4-methyl-pentoxy]-6-(2,6-dimethylphenyl)pyrimidin-2-yl]sulfamoyl]benzoic acid

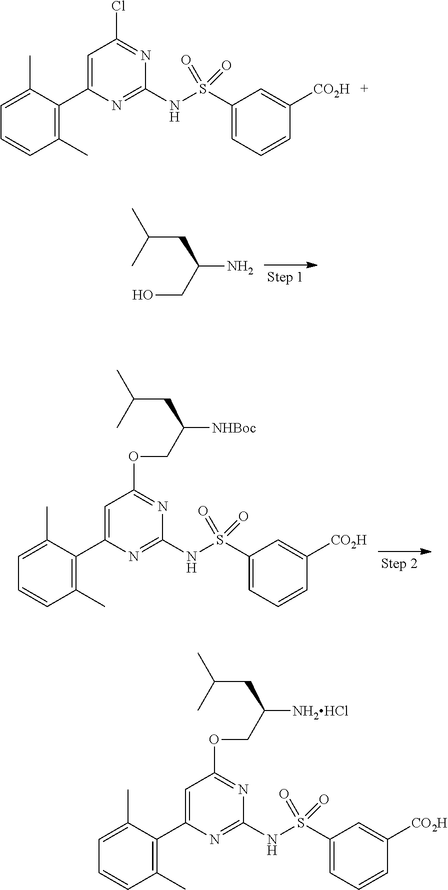

### Step 1: 3-[[4-[(2R)-2-(tert-Butoxycarbonylamino)-4-methyl-pentoxy]-6-(2,6-dimethylphenyl)pyrimidin-2-yl]sulfamoyl]benzoic acid

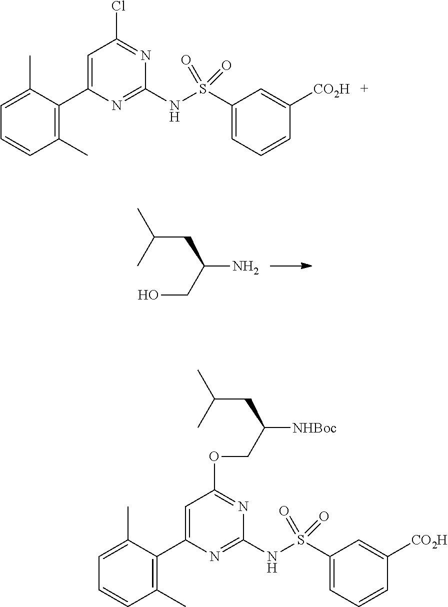

To a stirring solution of (2R)-2-amino-4-methyl-pentan-1-ol (12.419 g, 105.97 mmol) in anhydrous THE (200 mL) at room temperature under nitrogen was added sodium tert-butoxide (15.276 g, 158.95 mmol). The reaction mixture was stirred for 10 minutes and 3-[[4-chloro-6-(2,6-dimethylphenyl)pyrimidin-2-yl]sulfamoyl]benzoic acid (22.14 g, 52.983 mmol) was added. The reaction mixture was placed on a water bath preheated to 60° C. and stirred for 20 minutes. After cooling to room temperature, di-tert-butyl dicarbonate (69.381 g, 317.90 mmol) was added and the reaction mixture was stirred for 3 hours. The reaction was quenched with saturated aqueous ammonium chloride (150 mL). Volatiles were removed under vacuum and the aqueous layer was acidified to pH˜3 with 10% aqueous citric acid. The product was extracted with ethyl acetate (3×200 mL). The combined organic layers were washed with brine (80 mL), dried over anhydrous sodium sulfate and concentrated to a residual volume of ˜250 mL. The product was precipitated out into excess hexanes (750 mL) and collected by vacuum filtration. The obtained white solid was re-purified by silica gel chromatography using 0-40% acetone (0.15% acetic acid buffer) gradient in hexanes (0.15% acetic acid buffer) to afford 3-[[4-[(2R)-2-(tert-butoxycarbonylamino)-4-methyl-pentoxy]-6-(2,6-dimethylphenyl)pyrimidin-2-yl]sulfamoyl]benzoic acid (20.73 g, 61%) as a white solid. ESI-MS m/z calc. 598.2461, found 599.4 (M+1)⁺; Retention time: 5.85 minutes (LC Method S).

### Step 2: 3-[[4-[(2R)-2-Amino-4-methyl-pentoxy]-6-(2,6-dimethylphenyl)pyrimidin-2-yl]sulfamoyl]benzoic acid (hydrochloride salt)

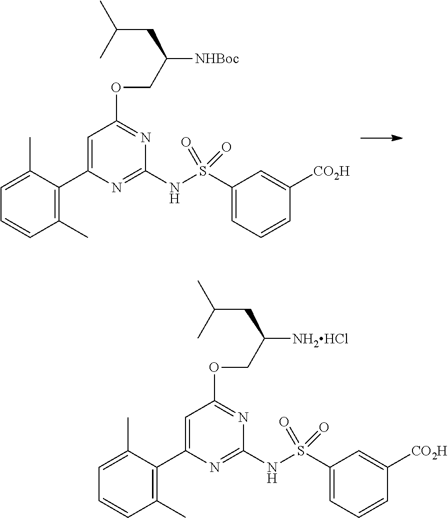

To a stirring solution of 3-[[4-[(2R)-2-(tert-butoxycarbonylamino)-4-methyl-pentoxy]-6-(2,6-dimethylphenyl)pyrimidin-2-yl]sulfamoyl]benzoic acid (20.73 g, 34.624 mmol) in DCM (200 mL) at room temperature was added HCl (87 mL of 4 M solution in 1,4-dioxane, 346.24 mmol). The reaction mixture was stirred for 2 hours. Volatiles were removed under vacuum and the obtained solid was triturated with diethyl ether (150 mL). After removal of the volatiles, the product was dried under vacuum to afford 3-[[4-[(2R)-2-amino-4-methyl-pentoxy]-6-(2,6-dimethylphenyl)pyrimidin-2-yl]sulfamoyl]benzoic acid (hydrochloride salt) (19.68 g, 100%) as a white solid. ¹H NMR (250 MHz, DMSO-d₆) δ 8.56-8.27 (m, 4H), 8.14 (t, J=6.8 Hz, 2H), 7.70 (t, J=7.8 Hz, 1H), 7.34-7.18 (m, 1H), 7.17-7.02 (m, 2H), 6.31 (s, 1H), 4.42-4.23 (m, 1H), 4.23-4.06 (m, 1H), 3.5-3.4 (m, 1H, overlapped with water), 2.01 (s, 6H), 1.82-1.31 (m, 3H), 1.02-0.78 (m, 6H). ESI-MS m/z calc. 498.1937, found 499.3 (M+1)⁺; Retention time: 1.63 minutes (LC Method T).

### Example C: Preparation of 3-[[4-[(2R)-2-amino-4,4-dimethyl-pentoxy]-6-(2,6-dimethylphenyl)pyrimidin-2-yl]sulfamoyl]benzoic acid

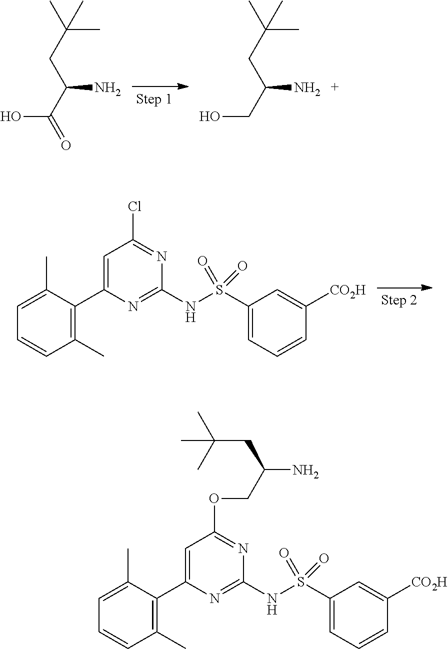

### Step 1: (2R)-2-Amino-4,4-dimethyl-pentan-1-ol

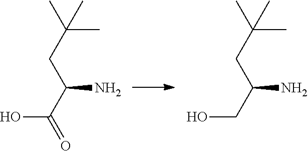

To a solution of (2R)-2-amino-4,4-dimethyl-pentanoic acid (15 g, 103.3 mmol) in THE (150 mL) at 0° C. was added borane-THF (260 mL of 1 M, 260.0 mmol) dropwise keeping the reaction temperature <10° C. The addition took approximately 30 min. The mixture was allowed to warm to ambient temperature and stirred for 22 h. The reaction was quenched with the slow addition of methanol (80 mL, 1.975 mol) and the solvent was removed in vacuo. The residue was co-evaporated 3× with methanol (200 mL, 4.937 mol) The crude residue was diluted with HCl (200 mL of 1 M, 200.0 mmol) and washed with 200 mL of MTBE. The aqueous phase was evaporated to remove residual organic solvent. The water was further removed in vacuo affording an off-white solid. The solid was further dried using an acetonitrile azeotrope. The solid was slurried in 200 mL of ACN and the precipitate collected using an M frit. The solid was air dried for 1 h, then in vacuo at 45° C. for 20 h to give (2R)-2-amino-4,4-dimethyl-pentan-1-ol (hydrochloride salt) (14.73 g, 85%). ¹H NMR (400 MHz, DMSO-d₆) δ 7.80 (s, 3H), 5.36 (t, J=5.1 Hz, 1H), 3.59 (dt, J=11.7, 4.1 Hz, 1H), 3.42-3.34 (m, 1H), 3.10 (dq, J=7.7, 3.8 Hz, 1H), 1.46 (dd, J=14.5, 7.1 Hz, 1H), 1.33 (dd, J=14.5, 3.5 Hz, 1H), 0.91 (s, 9H). ESI-MS m z calc. 131.13101, found 132.1 (M+1)⁺; Retention time: 0.51 minutes (LC method A).

### Step 2: 3-[[4-[(2R)-2-Amino-4,4-dimethyl-pentoxy]-6-(2,6-dimethylphenyl)pyrimidin-2-yl]sulfamoyl]benzoic acid

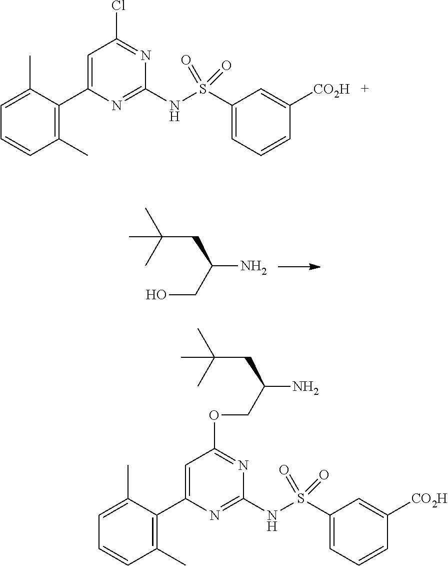

3-[[4-Chloro-6-(2,6-dimethylphenyl)pyrimidin-2-yl]sulfamoyl]benzoic acid (20 g, 47.862 mmol) was suspended in a mixture of 2-methyltetrahydrofuran (80 mL) and DMF (20 mL) and the solution was cooled to −5° C. Sodium tert-butoxide (23 g, 239.33 mmol) was then dissolved in 2-methyltetrahydrofuran (100 mL), cooled to 5° C. and added over 10 minutes, followed by (2R)-2-amino-4,4-dimethyl-pentan-1-ol (hydrochloride salt) (8.02 g, 47.830 mmol). The reaction was then warmed to 10° C. and stirred for 4 hours. It was then cooled to 0° C. and quenched by adding an aqueous solution of hydrochloric acid (2 M, 200 mL) over 10 minutes. The phases were separated, and the aqueous phase extracted with 2-methyltetrahydrofuran (200 mL). The organic phases were combined and washed with an aqueous solution of sodium chloride (15% w/w, 2×200 mL), dried over sodium sulfate (60 g), filtered and evaporated to dryness. The solid was then triturated using ethyl acetate (200 mL) for 16 hours, filtered, washed with ethyl acetate and dried in a vacuum oven at 50° C. for 20 hours to give 3-[[4-[(2R)-2-amino-4,4-dimethyl-pentoxy]-6-(2,6-dimethylphenyl)pyrimidin-2-yl]sulfamoyl]benzoic acid (hydrochloride salt) (22.29 g, 80%). ¹H NMR (400 MHz, DMSO-d₆) δ 13.26 (br. s., 2H), 8.45 (t, J=1.6 Hz, 1H), 8.28-8.06 (m, 5H), 7.69 (t, J=7.8 Hz, 1H), 7.31-7.21 (m, 1H), 7.13 (d, J=7.6 Hz, 2H), 6.29 (br. s., 1H), 4.30 (dd, J=11.7, 2.7 Hz, 1H), 4.10 (dd, J=11.5, 7.1 Hz, 1H), 3.56 (br. s., 1H), 2.13-1.90 (s, 6H), 1.62-1.47 (m, 2H), 0.94 (s, 9H). ESI-MS m/z calc. 512.20935, found 513.0 (M+1)⁺; Retention time: 2.334 minutes; LC method U.

### Example D: Preparation of 3-[[4-[(2R)-2-amino-5,5,5-trifluoro-4,4-dimethyl-pentoxy]-6-(2,6-dimethylphenyl)pyrimidin-2-yl]sulfamoyl]benzoic acid

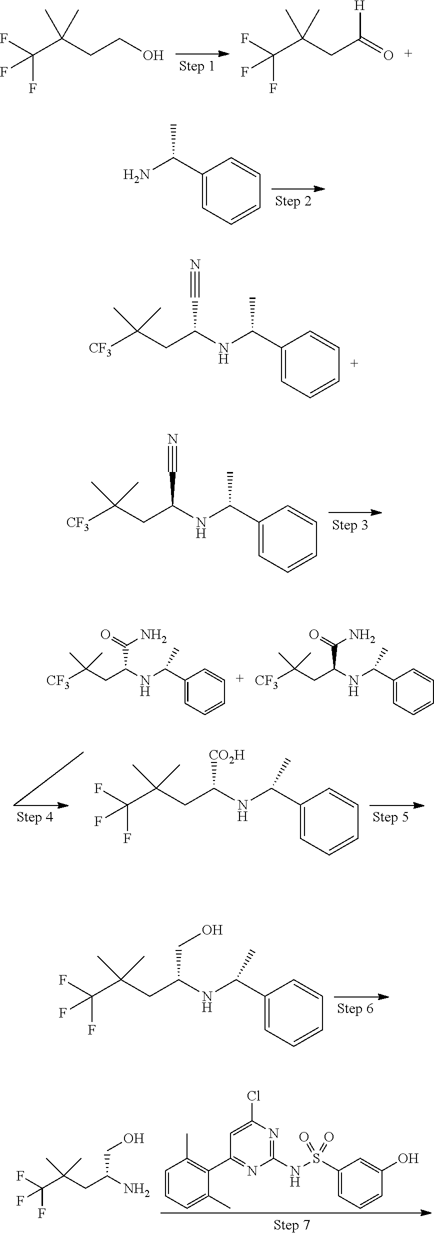

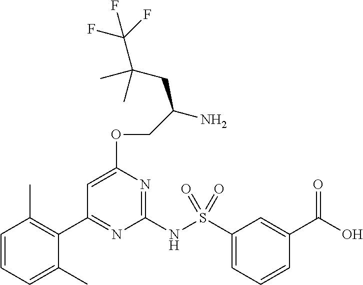 [SMILES: Cc1cccc(C)c1-c1cc(OC[C@H](N)CC(C)(C)C(F)(F)F)nc(NS(=O)(=O)c2cccc(C(=O)O)c2)n1]

### Step 1: 4,4,4-Trifluoro-3,3-dimethyl-butanal

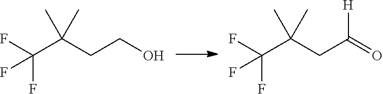

A 1 L three-neck flask was charged with 4,4,4-trifluoro-3,3-dimethyl-butan-1-ol (8.987 g, 57.555 mmol), DCM (63 mL), water (63 mL), NaBr (544 mg, 5.2870 mmol), sodium bicarbonate (12.32 g, 146.66 mmol) and TEMPO (92 mg, 0.5888 mmol). The mixture was cooled with ice-water bath. An aqueous solution of NaOCl (47 mL of 1.31 M, 61.570 mmol) was added dropwise over 2 h at 2.5-4.4° C. After the addition, the mixture was stirred for 10 min. The two layers was separated. The aqueous phase was extracted with DCM (2×15 mL). The combined organic layers were dried with sodium sulfate and filtered to give 113.7 g (about 80 mL) of crude product in DCM, which was used directly the next step. ¹H NMR (300 MHz, CDCl₃) δ 9.82-9.78 (m, 1H), 2.54 (d, J=2.6 Hz, 2H), 1.28 (s, 6H). ¹⁹F NMR (282 MHz, CDCl₃) δ-79.11 (s, 3F).

### Step 2: (2R)-5,5,5-Trifluoro-4,4-dimethyl-2-[[(1R)-1-phenylethyl]amino]pentanenitrile and (2S)-5,5,5-trifluoro-4,4-dimethyl-2-[[(1R)-1-phenylethyl]amino]pentanenitrile

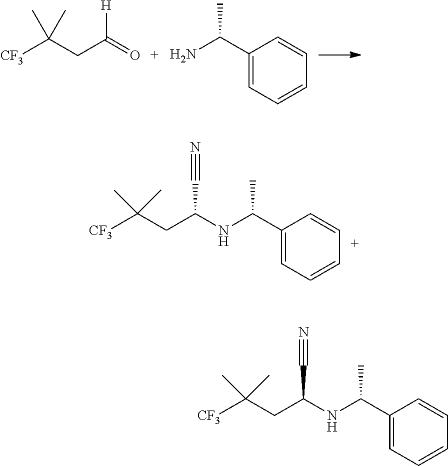

To a DCM (80 mL) solution of 4,4,4-trifluoro-3,3-dimethyl-butanal (113.7 g, 57.549 mmol) (purity about 7.8%) was added MeOH (110 mL). The mixture was cooled with ice-water bath. (1R)-1-phenylethanamine (8.46 g, 69.814 mmol) was added, followed by acetic acid (4.41 g, 73.436 mmol). The mixture was stirred at 0° C. for 10 min, then NaCN (3.56 g, 72.642 mmol) was added. The mixture was allowed to warm to rt slowly and stirred overnight. The reaction mixture was cooled to 0° C. and a solution of potassium carbonate (4 g) in water (20 mL) was added dropwise, followed by brine (40 mL). The mixture was extracted with DCM (2×100 mL). The organic layers were dried with sodium sulfate, filtered and concentrated. The residue was purified by flash chromatography (120 g silica gel, heptanes/EtOAc 0-30%) to afford a 4:1 mixture of (2R)-5,5,5-trifluoro-4,4-dimethyl-2-[[(1R)-1-phenylethyl]amino]pentanenitrile and (2S)-5,5,5-trifluoro-4,4-dimethyl-2-[[(1R)-1-phenylethyl]amino]pentanenitrile (14.87 g, 91%) as a colorless oil. ESI-MS m/z calc. 284.15002, found 285.2 (M+1)⁺; Retention time: 3.38 minutes; LC method U.

### Step 3: (2R)-5,5,5-Trifluoro-4,4-dimethyl-2-[[(1R)-1-phenylethyl]amino]pentanamide and (2S)-5,5,5-trifluoro-4,4-dimethyl-2-[[(1R)-1-phenylethyl]amino]pentanamide

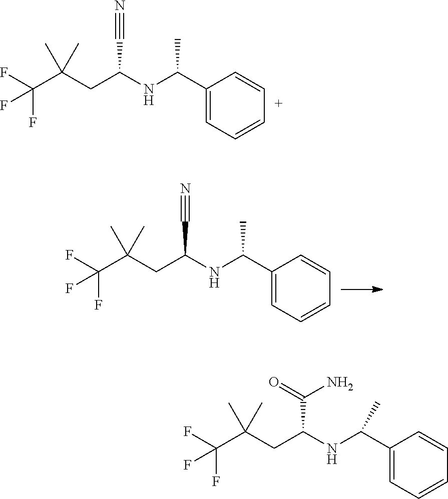 [SMILES: C[C@@H](N[C@@H](C#N)CC(C)(C)C(F)(F)F)c1ccccc1 ; C[C@@H](N[C@H](C#N)CC(C)(C)C(F)(F)F)c1ccccc1 ; C[C@@H](N[C@H](CC(C)(C)C(F)(F)F)C(N)=O)c1ccccc1]

To a solution of a 4:1 mixture of (2R)-5,5,5-trifluoro-4,4-dimethyl-2-[[(1R)-1-phenylethyl]amino]pentanenitrile and (2S)-5,5,5-trifluoro-4,4-dimethyl-2-[[(1R)-1-phenylethyl]amino]pentanenitrile (14.87 g, 52.300 mmol) in DCM (105 mL) was added sulfuric acid (56.3 g, 551.06 mmol). The mixture was stirred at rt overnight, poured on crude ice (200 g) and neutralized to pH 9 with 28% NH₃ in water (100 mL). The mixture was extracted with DCM (500 mL). The organic layer was dried with sodium sulfate, filtered and concentrated. The residue was purified by flash chromatography (330 g silica gel, heptanes/EtOAc 20-50%) to afford (2R)-5,5,5-trifluoro-4,4-dimethyl-2-[[(1R)-1-phenylethyl]amino]pentanamide (10.77 g, 68%) as a white solid. ¹H NMR (300 MHz, CDCl₃) δ 7.39-7.22 (m, 5H), 6.35 (br. s., 1H), 5.55 (br. s., 1H), 3.65 (q, J=6.5 Hz, 1H), 2.93 (dd, J=7.6, 3.8 Hz, 1H), 1.87 (dd, J=15.0, 3.8 Hz, 1H), 1.65-1.56 (m, 2H), 1.35 (d, J=6.5 Hz, 3H), 1.04 (s, 3H), 1.00 (s, 3H). ¹⁹F NMR (282 MHz, CDCl₃) δ-78.77 (s, 3F). 99.4% de by 19F NMR.

### Step 4: (2R)-5,5,5-Trifluoro-4,4-dimethyl-2-[[(1R)-1-phenylethyl]amino]pentanoic acid

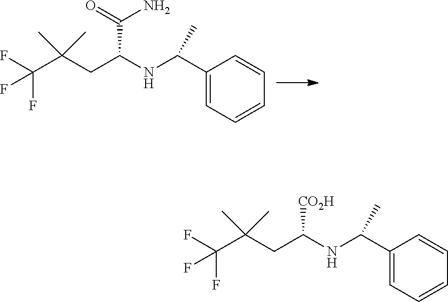 [SMILES: C[C@@H](N[C@H](CC(C)(C)C(F)(F)F)C(N)=O)c1ccccc1 ; C[C@@H](N[C@H](CC(C)(C)C(F)(F)F)C(=O)O)c1ccccc1]

To a solution of (2R)-5,5,5-trifluoro-4,4-dimethyl-2-[[(1R)-1-phenylethyl]amino]pentanamide (11.35 g, 37.541 mmol) in HOAc (50 mL) was added conc. HCl (65 mL of 11.8 M, 767.00 mmol), followed by water (50 mL). A white precipitate appeared. The mixture was heated at 100° C. for 66 h. More conc. HCl (40 mL of 11.8 M, 472.00 mmol) and HOAc (10 mL) were added. The mixture was stirred at 100° C. overnight. More HCl in water (20 mL of 6 M, 120.00 mmol) was added. After 7 h at 100° C., more HCl in water (20 mL of 6 M, 120.00 mmol) was added. The mixture was stirred at 100° C. overnight. It became a clear solution. More HCl in water (20 mL of 6 M, 120.00 mmol) was added. The mixture was stirred at 100° C. for 7 h, more HCl in water (20 mL of 6 M, 120.00 mmol) was added. The mixture was stirred at 100° C. overnight. The mixture was concentrated and co-evaporated with water (50 mL). The residue (17 g) was mixed with water (25 mL) at 50° C. for 20 min, cooled with ice-water bath for 20 min and filtered. The crude product was mixed with 1,4-dioxane (60 mL). The mixture was concentrated and dried on vacuum overnight to give (2R)-5,5,5-trifluoro-4,4-dimethyl-2-[[(1R)-1-phenylethyl]amino]pentanoic acid (hydrochloride salt) (13.04 g, 97%) as an off-white solid. ¹H NMR (300 MHz, DMSO-d₆) δ 10.09 (br. s., 1H), 7.54-7.31 (m, 5H), 7.29-7.05 (m, 1H), 4.07 (q, J=5.9 Hz, 1H), 3.16-2.98 (m, 1H), 2.08-1.83 (m, 2H), 1.49 (d, J=6.5 Hz, 3H), 0.99 (s, 3H), 0.92 (s, 3H). ¹⁹F NMR (282 MHz, DMSO-d₆) δ-78.28 (s, 3F). ESI-MS m/z calc. 303.14462, found 304.2 (M+1)⁺; Retention time: 1.98 minutes; LC method U.

### Step 5: (2R)-5,5,5-Trifluoro-4,4-dimethyl-2-[[(1R)-1-phenylethyl]amino]pentan-1-ol

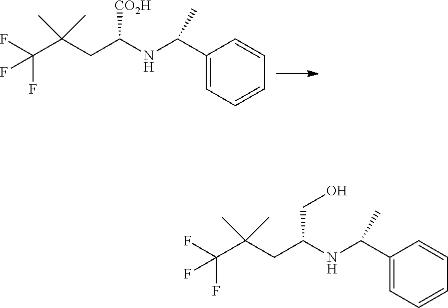 [SMILES: C[C@@H](N[C@H](CC(C)(C)C(F)(F)F)C(=O)O)c1ccccc1 ; C[C@@H](N[C@@H](CO)CC(C)(C)C(F)(F)F)c1ccccc1]

To a suspension of (2R)-5,5,5-trifluoro-4,4-dimethyl-2-[[(1R)-1-phenylethyl]amino]pentanoic acid (hydrochloride salt) (13.04 g, 36.267 mmol) in THE (200 mL) at 35° C. was added LAH in THE (100 mL of 1 M, 100.00 mmol) dropwise. The mixture was stirred at 40° C. for 2 h, cooled to 10° C. with ice-water bath and diluted with THE (200 mL). A mixture of water (3.8 g) and THE (50 mL) was added dropwise, followed by 25% aqueous NaOH (3.8 g) and water (10 g). The resulting mixture was stirred at rt for 30 min and at 50° C. for 1 h, filtered and washed with warm THF. The filtrate was concentrated to give 12.02 g of product (free amine) as a colorless oil. ¹H NMR (300 MHz, CDCl₃) δ 7.37-7.24 (m, 5H), 3.82 (q, J=6.5 Hz, 1H), 3.72-3.67 (m, 1H), 3.21 (dd, J=10.6, 4.7 Hz, 1H), 2.67 (quin, J=4.6 Hz, 1H), 1.66 (dd, J=14.7, 5.9 Hz, 1H), 1.54-1.45 (m, 1H), 1.36 (d, J=6.5 Hz, 3H), 1.03 (s, 3H), 0.97 (s, 3H). ¹⁹F NMR (282 MHz, CDCl₃) δ-78.83 (s, 3F). The above crude product (12.02 g) was dissolved in diethyl ether (20 mL) and diluted with heptanes (80 mL) and cooled in an ice-water bath. HCl in 1,4-dioxane (10.5 mL of 4 M, 42.000 mmol) was added dropwise. The mixture was stirred at rt for 30 min and filtered to give (2R)-5,5,5-trifluoro-4,4-dimethyl-2-[[(1R)-1-phenylethyl]amino]pentan-1-ol (hydrochloride salt) (11.56 g, 98%) as a white solid. ¹H NMR (300 MHz, DMSO-d₆) δ 9.57 (br. s., 1H), 9.25 (t, J=9.8 Hz, 1H), 7.80-7.59 (m, 2H), 7.53-7.32 (m, 3H), 5.63 (br. s., 1H), 4.58 (t, J=6.3 Hz, 1H), 3.81-3.65 (m, 1H), 3.64-3.51 (m, 1H), 2.91-2.74 (m, 1H), 1.98-1.85 (m, 1H), 1.85-1.74 (m, 1H), 1.63 (d, J=6.8 Hz, 3H), 0.91 (s, 3H), 0.88 (s, 3H). ¹⁹F NMR (282 MHz, DMSO-d₆) δ-77.71 (s, 3F). ESI-MS m/z calc. 289.16534, found 290.2 (M+1)⁺; Retention time: 2.08 minutes; LC method U.

### Step 6: (2R)-2-Amino-5,5,5-trifluoro-4,4-dimethyl-pentan-1-ol

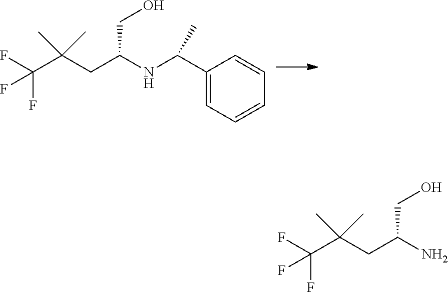 [SMILES: C[C@@H](N[C@@H](CO)CC(C)(C)C(F)(F)F)c1ccccc1 ; CC(C)(C[C@@H](N)CO)C(F)(F)F]

To a solution of (2R)-5,5,5-trifluoro-4,4-dimethyl-2-[[(1R)-1-phenylethyl]amino]pentan-1-ol (hydrochloride salt) (11.56 g, 35.482 mmol) in EtOH (200 mL) was added 10% palladium on carbon, 50% wet (5 g, 2.3492 mmol). The mixture was hydrogenated in a Parr shaker hydrogenation apparatus at 40 psi of hydrogen at rt for 9 h. More 10% palladium on carbon, 50% wet (1 g, 0.4698 mmol) was added. The mixture was shaken at 40 psi for 7 h. The mixture was filtered through Celite and washed with EtOH. The filtrate was concentrated. The residue (7.9 g) was triturated with a mixture of 2-methyltetrahydrofuran (28 mL) and heptanes (200 mL) and stirred overnight. The mixture was filtered, and the white solid was dried on vacuum to give (2R)-2-amino-5,5,5-trifluoro-4,4-dimethyl-pentan-1-ol (hydrochloride salt) (7.66 g, 93%) as a white solid. ¹H NMR (300 MHz, DMSO-d₆) δ 8.08 (br. s., 3H), 5.46 (t, J=5.0 Hz, 1H), 3.67-3.52 (m, 1H), 3.43 (dt, J=11.7, 5.8 Hz, 1H), 3.29-3.16 (m, 1H), 1.88-1.73 (m, 1H), 1.72-1.58 (m, 1H), 1.15 (s, 3H), 1.10 (s, 3H). ¹⁹F NMR (282 MHz, DMSO-d₆) δ-78.07 (s, 3F). ESI-MS m/z calc. 185.10275, found 186.2 (M+1)⁺; Retention time: 0.64 minutes; LC method U.

### Step 7: 3-[[4-[(2R)-2-Amino-5,5,5-trifluoro-4,4-dimethyl-pentoxy]-6-(2,6-dimethylphenyl)pyrimidin-2-yl]sulfamoyl]benzoic acid

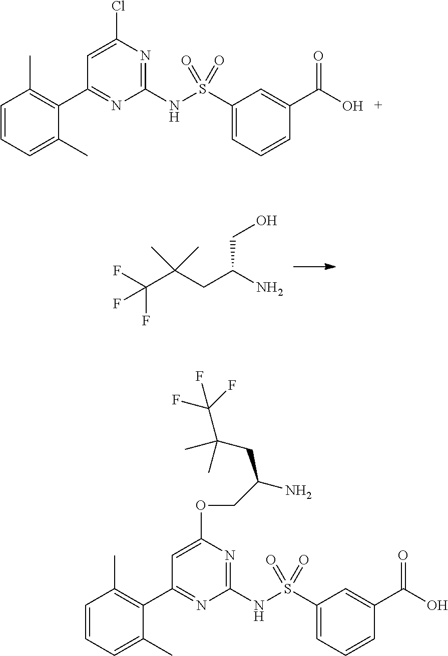 [SMILES: Cc1cccc(C)c1-c1cc(Cl)nc(NS(=O)(=O)c2cccc(C(=O)O)c2)n1 ; CC(C)(C[C@@H](N)CO)C(F)(F)F ; Cc1cccc(C)c1-c1cc(OC[C@H](N)CC(C)(C)C(F)(F)F)nc(NS(=O)(=O)c2cccc(C(=O)O)c2)n1]

3-[[4-Chloro-6-(2,6-dimethylphenyl)pyrimidin-2-yl]sulfamoyl]benzoic acid (6.12 g, 14.65 mmol) and (2R)-2-amino-5,5,5-trifluoro-4,4-dimethyl-pentan-1-ol (hydrochloride salt) (3.27 g, 14.75 mmol) were combined in THE (30 mL) and the resulting suspension was cooled in a water-ice bath. Sodium tert-butoxide (5.63 g, 58.58 mmol) was added inducing rapid partial dissolution of the solid. After 5 minutes, the cooling bath was removed, and the reaction was stirred at room temperature for 1 hour (90% conversion). More (2R)-2-amino-5,5,5-trifluoro-4,4-dimethyl-pentan-1-ol (hydrochloride salt) (363 mg, 1.638 mmol) was added and the mixture was stirred for one hour (no change). More sodium tert-butoxide (744 mg, 7.742 mmol) was added and the mixture was stirred for 40 min (96% conversion). Ethyl acetate (100 mL), HCl (90 mL of 1 M, 90.00 mmol) and brine (50 mL) were added and the resulting two phases were separated. The organic phase was washed with brine (50 mL), dried over sodium sulfate and concentrated. The residue was triturated in EtOAc/MeOH/Hexanes and the solvents were evaporated to give 3-[[4-[(2R)-2-amino-5,5,5-trifluoro-4,4-dimethyl-pentoxy]-6-(2,6-dimethylphenyl)pyrimidin-2-yl]sulfamoyl]benzoic acid (hydrochloride salt) (8.88 g, 93%) as a cream solid. ¹H NMR (400 MHz, DMSO-d₆) δ 13.15 (very broad s, 1H), 8.61-8.30 (m, 4H), 8.14 (dd, J=7.9, 1.9 Hz, 2H), 7.69 (t, J=7.8 Hz, 1H), 7.31-7.20 (m, 1H), 7.12 (d, J=7.6 Hz, 2H), 6.33 (s, 1H), 4.43 (dd, J=11.9, 3.3 Hz, 1H), 4.29-4.15 (m, 1H), 3.74 (s, 1H), 2.06-1.94 (broad m, 6H), 1.94-1.85 (m, 2H), 1.22 (s, 3H), 1.16 (s, 3H). ESI-MS m/z calc. 566.1811, found 567.62 (M+1)⁺; Retention time: 1.13 minutes (LC method A).

### Example E: Preparation of 3-[[4-[(2R)-2-amino-3-[1-(trifluoromethyl)cyclopropyl]propoxy]-6-(2,6-dimethylphenyl)pyrimidin-2-yl]sulfamoyl]benzoic acid

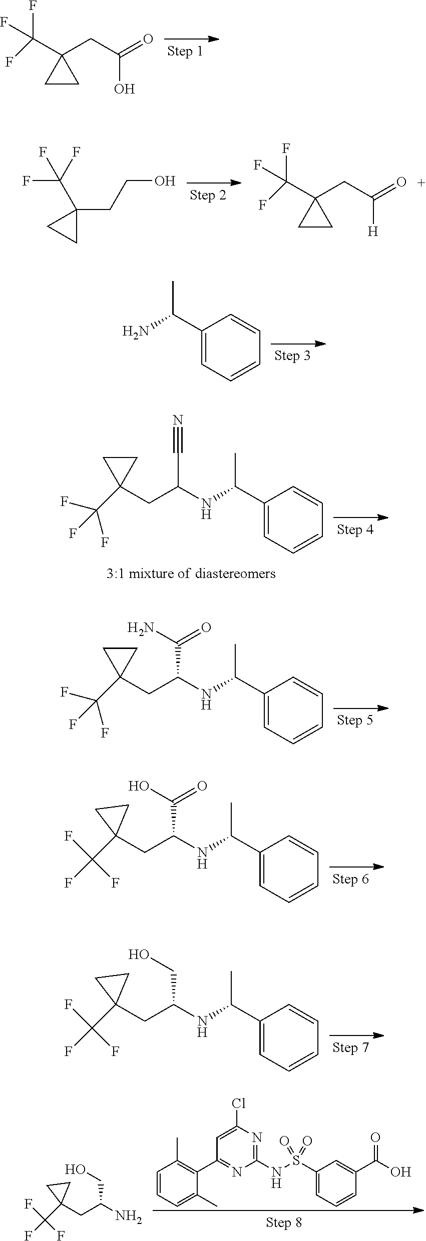

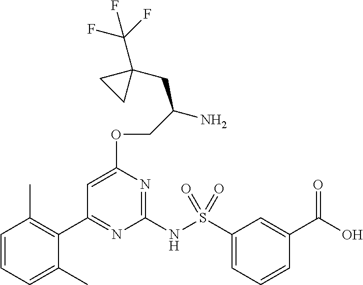 [SMILES: Cc1cccc(C)c1-c1cc(OC[C@H](N)CC2(C(F)(F)F)CC2)nc(NS(=O)(=O)c2cccc(C(=O)O)c2)n1]

### Step 1: 2-[1-(Trifluoromethyl)cyclopropyl]ethanol

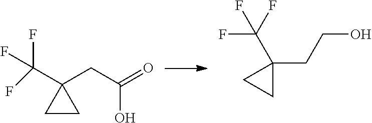 [SMILES: O=C(O)CC1(C(F)(F)F)CC1 ; OCCC1(C(F)(F)F)CC1]

LAH (49.868 g, 1.3139 mol) was added to THE (1700 mL) under nitrogen and the mixture was stirred for 30 minutes before being cooled to 0° C. 2-[1-(trifluoromethyl)cyclopropyl]acetic acid (190.91 g, 1.0107 mol) in THE (500 mL) was added dropwise while controlling the temperature <5° C. The mixture was allowed to warm up to room temperature and stirred for 24 hours. The resulting suspension was cooled to 0° C., water (50 mL) was added very slowly, followed by 15% w/w sodium hydroxide (50 mL) and water (150 mL). The mixture was stirred at 0° C. for 30 minutes, and filtered through Celite pad, the filter cake was washed with THF (2×500 mL). The combined filtrates were evaporated in vacuo to give 2-[1-(trifluoromethyl)cyclopropyl]ethanol (160.27 g, 98%) as amber oil containing ˜5% w/w of THF (by NMR). ¹H NMR (250 MHz, DMSO-d₆) δ 4.57 (t, J=5.2 Hz, 1H), 3.55-3.39 (m, 2H), 1.74 (t, J=7.3 Hz, 2H), 1.00-0.58 (m, 4H).

### Step 2: 2-[1-(Trifluoromethyl)cyclopropyl]acetaldehyde

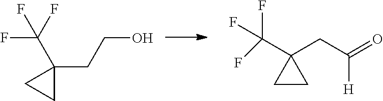 [SMILES: OCCC1(C(F)(F)F)CC1 ; O=CCC1(C(F)(F)F)CC1]

To a solution of 2-[1-(trifluoromethyl)cyclopropyl]ethanol (80 g, 467.1 mmol) in methylene chloride (1.1 L) was stirred at room temperature and treated with Dess-Martin periodinane (250 g, 589.4 mmol) portionwise (exothermic! cooled in ice bath and kept T<15° C.). To the mixture was added water (12 mL, 666.1 mmol) slowly added over 0.5 h (exothermic during addition up to 33° C., kept between 20 and 33° C. by cooling with cold water) giving a thick suspension. After the addition, the pale-yellow fine suspension was stirred at room temperature for 18 h. The yellow suspension was diluted with diethylether (500 mL) (yellow suspension) and stirred for 30 min. The slurry was filtered over Celite and the precipitate washed with 100 mL of Diethylether. diethylether. The organic phase was carefully treated with a saturated aqueous solution of sodium carbonate (500 ml, strong gas evolution, pH˜10 at the end). The three-phase mixture was stirred at room temperature for 1 h and the solid was removed by filtration (large glass frit). The phases (yellow cloudy Diethylether phase, colorless water phase) were separated and the organic phase was washed once more with a saturated aqueous solution of sodium carbonate (250 mL), once with 1M sodium thiosulfate (250 mL) and once with brine (250 mL). The aqueous phases were back extracted once with diethyl ether (150 mL) and the combined organic phases were dried, filtered and evaporated to give 2-[1-(trifluoromethyl)cyclopropyl]acetaldehyde (40 g, 56%) as a yellow liquid.

### Step 3: 2-[[(1R)-1-Phenylethyl]amino]-3-[1-(trifluoromethyl)cyclopropyl]propanenitrile

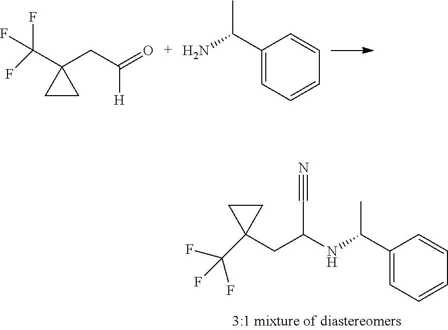

2-[1-(Trifluoromethyl)cyclopropyl]acetaldehyde (102 g, 670.5 mmol) in MeOH (700 mL) was treated with (1R)-1-phenylethanamine (86 mL, 667.1 mmol) and cooled in an ice bath. The solution was treated with acetic acid (38 mL, 668.2 mmol), stirred for 20 min in the ice bath, then solid NaCN (CAUTION, 33 g, 673.4 mmol) was added in one portion and the suspension was stirred in the melting ice bath for 14 hours. The solution was concentrated under reduced pressure (CAUTION, HCN!, the exhaust from the pump was running through a bleach trap) and the residue was extracted with MTBE (1000 mL) and saturated sodium carbonate/water 1:1 (1000 mL) and washed with brine (350 mL). The aqueous phases were back extracted once with MTBE (250 mL) and the combined organic phases were dried, filtered and evaporated to give 2-[[(1R)-1-phenylethyl]amino]-3-[1-(trifluoromethyl)cyclopropyl]propanenitrile (180.8 g, 96%) as 3:1 mixture of diastereomers. ESI-MS m/z calc. 282.13437, found 283.0 (M+1)⁺; Retention time: 1.69 minutes (major isomer) and 1.62 minutes (minor isomer), LC method A.

### Step 4: (2R)-2-[[(1R)-1-Phenylethyl]amino]-3-[1-(trifluoromethyl)cyclopropyl]propenamide

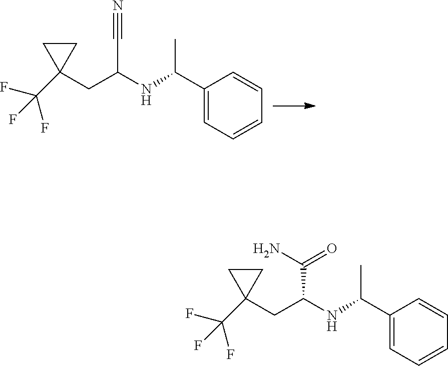 [SMILES: C[C@@H](NC(C#N)CC1(C(F)(F)F)CC1)c1ccccc1 ; C[C@@H](N[C@H](CC1(C(F)(F)F)CC1)C(N)=O)c1ccccc1]

In a 2 L flask equipped with mechanical stirring and a temperature probe, sulfuric acid (285 mL of 18 M, 5.130 mol) was added it was cooled in an ice bath. At an internal temperature of 5° C., a solution of 2-[[(1R)-1-phenylethyl]amino]-3-[1-(trifluoromethyl)cyclopropyl]propanenitrile (180.8 g, 640.4 mmol, 3:1 mixture of diastereomers) in DCM (900 mL) was added dropwise over 20 minutes. The ice bath was removed, and the deep orange emulsion was stirred at room temperature for 18 h and at 30-40° C. for 2 h. The deep orange emulsion was carefully added to a mixture of ice and water (2.2 L) under mechanical stirring to give a yellow three phase mixture which was basified by slow addition of ammonium hydroxide (1.33 L of 30% w/w, 10.25 mol) under ice cooling (very exothermic, internal temperature kept between 10 and 25° C. by adding ice). The yellow emulsion was stirred for 10 minutes at room temperature (pH˜10), diluted with DCM (500 mL) and the phases were separated. The aqueous phase was washed twice more with DCM (400 and 200 mL) and the combined organic phases were washed once with water/brine 1:1 (500 mL). The DCM phase was dried, filtered and evaporated to give crude 2-[[(1R)-1-phenylethyl]amino]-3-[1-(trifluoromethyl)cyclopropyl]propanamide (189.5 g, 99%) as a yellow-orange oil. ESI-MS m z calc. 300.14496, found 301.0 (M+1)⁺; Retention time: 1.40 minutes (major isomer) and 1.50 minutes (minor isomer) (3:1 mixture of diastereomers). The product was dissolved in ethanol (1.5 L) and it was treated quickly with HCl (240 mL of 4 M, 960.0 mmol) (4M in dioxane) and the resulting thick suspension was stirred at room temperature overnight under mechanic stirring. The solid was collected by filtration, washed with cold ethanol and dried under vacuum with a nitrogen bleed at 40-45° C. to give (2R)-2-[[(1R)-1-phenylethyl]amino]-3-[1-(trifluoromethyl)cyclopropyl]propanamide (hydrochloride salt) (147 g, 68%). ¹H NMR (499 MHz, DMSO-d₆) δ 9.74 (d, J=67.9 Hz, 2H), 8.16-7.94 (m, 1H), 7.86 (s, 1H), 7.64-7.51 (m, 2H), 7.51-7.34 (m, 3H), 4.22 (s, 1H), 3.46-3.37 (m, 1H), 2.45 (d, J=15.9 Hz, 1H), 1.85 (dd, J=15.1, 10.4 Hz, 1H), 1.58 (d, J=6.7 Hz, 3H), 0.89 (pd, J=9.6, 9.2, 4.3 Hz, 2H), 0.84-0.66 (m, 2H). ESI-MS m/z calc. 300.14496, found 301.0 (M+1)⁺; Retention time: 1.40 minutes (major isomer) and 1.40 minutes (minor isomer), 97:3 mixture of diastereomers (LC method V).

### Step 5: (2R)-2-[[(1R)-1-Phenylethyl]amino]-3-[1-(trifluoromethyl)cyclopropyl]propanoic acid

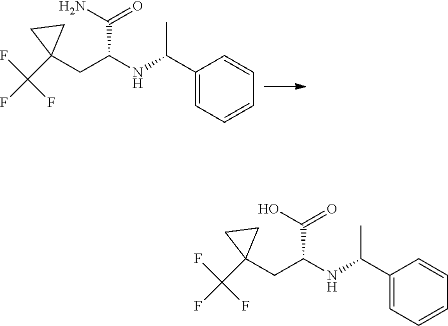 [SMILES: C[C@@H](N[C@H](CC1(C(F)(F)F)CC1)C(N)=O)c1ccccc1 ; C[C@@H](N[C@H](CC1(C(F)(F)F)CC1)C(=O)O)c1ccccc1]

In a 5 L flask equipped with mechanical stirring, (2R)-2-[[(1R)-1-phenylethyl]amino]-3-[1-(trifluoromethyl)cyclopropyl]propanamide (hydrochloride salt) (147 g, 436.5 mmol) was added to acetic acid (735 mL) under stirring and the thick colorless suspension was treated with HCl (1.3 L of 12 M, 15.60 mol). The colorless suspension was carefully heated to 60-65° C. (strong foaming, acetic acid (145 mL) was added) and the suspension was stirred at 60-65° C. for 16 h. The suspension was then slowly heated to 100° C. (over 4 h, strong foaming) and the resulting solution was stirred at 100° C. for another 20 h. The pale-yellow solution was concentrated under reduced pressure at 65° C. to a semisolid mass and it was treated with water (1.5 L). The thick suspension was heated to 70-80° C. and left to cool to room temperature under stirring for 2 h. The solid was collected by filtration, washed with water and sucked dry overnight. The wet solid was further dried under reduced pressure at 50-60° C. for 4 h to give (2R)-2-[[(1R)-1-phenylethyl]amino]-3-[1-(trifluoromethyl)cyclopropyl]propanoic acid (hydrochloride salt) (135 g, 92%) as an off-white solid. ESI-MS m/z calc. 301.12897, found 302.0 (M+1)⁺; Retention time: 1.82 minutes; (LC method V).

### Step 6: (2R)-2-[[(1R)-1-phenylethyl]amino]-3-[1-(trifluoromethyl)cyclopropyl]propan-1-ol

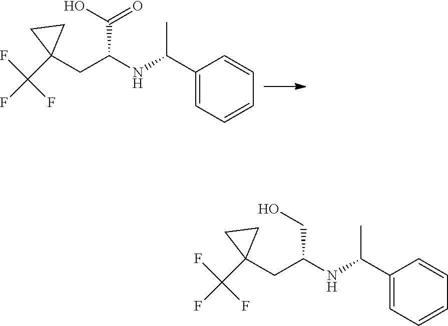 [SMILES: C[C@@H](N[C@H](CC1(C(F)(F)F)CC1)C(=O)O)c1ccccc1 ; C[C@@H](N[C@@H](CO)CC1(C(F)(F)F)CC1)c1ccccc1]

In a 5 L flask equipped with mechanical stirring and under dry nitrogen atmosphere, (2R)-2-[[(1R)-1-phenylethyl]amino]-3-[1-(trifluoromethyl)cyclopropyl]propanoic acid (hydrochloride salt) (135 g, 399.7 mmol) was suspended in THE (2 L) (thick suspension). It was heated to 35-40° C. and LAH (47.3 g, 1.214 mol) (pellets) was slowly added over 1 hour, while keeping the internal temperature between 30 and 40° C. by external cooling. The mixture was stirred for 1 hour at 30-40° C. (almost no hydrogen evolution anymore, grey suspension, most starting material in solution) and it was heated at 50-55° C. for 1 h. The grey suspension was left stirring in the cooling heating mantel overnight. The grey suspension was cooled in an ice bath and quenched by careful addition of water (44 mL, 2.442 mol), NaOH (41 mL of 6 M, 246.0 mmol) and water (44 mL, 2.442 mol) (high exotherm with first water addition, kept between 5° C. and 30° C. by cooling). The grey suspension was heated to 50-55° C. for 1 h, by which time a colorless suspension was obtained. The warm suspension was filtered over a pad of Celite covered over magnesium sulfate. The solids were washed with hot THF and evaporated to give crude (2R)-2-[[(1R)-1-phenylethyl]amino]-3-[1-(trifluoromethyl)cyclopropyl]propan-1-ol (121 g, 105%) as an oil. The crude was dissolved in diethyl ether (1 L, clear solution) and slowly treated with HCl (101 mL of 4 M, 404.0 mmol) (4M in dioxane) under cooling. The resulting thick suspension was stirred at room temperature for 1 h, the solid collected by filtration, washed with diethyl ether and dried under reduced pressure at 40-45° C. with a nitrogen bleed to give (2R)-2-[[(1R)-1-phenylethyl]amino]-3-[1-(trifluoromethyl)cyclopropyl]propan-1-ol (hydrochloride salt) (126.6 g, 98%) as an off-white solid. ¹H NMR (500 MHz, DMSO-d₆) δ 9.34 (s, 2H), 7.66 (d, J=7.4 Hz, 2H), 7.43 (dt, J=25.1, 7.4 Hz, 3H), 5.59 (s, 1H), 4.58 (q, J=6.6 Hz, 1H), 3.83 (d, J=12.6 Hz, 1H), 3.62-3.54 (m, 1H), 2.89 (s, 1H), 2.33-2.24 (m, 1H), 1.67-1.51 (m, 4H), 0.97-0.81 (m, 3H), 0.71 (s, 1H). ESI-MS m/z calc. 287.1497, found 288.0 (M+1)⁺; Retention time: 0.99 minutes (LC method A).

### Step 7: (2R)-2-Amino-3-[1-(trifluoromethyl)cyclopropyl]propan-1-ol

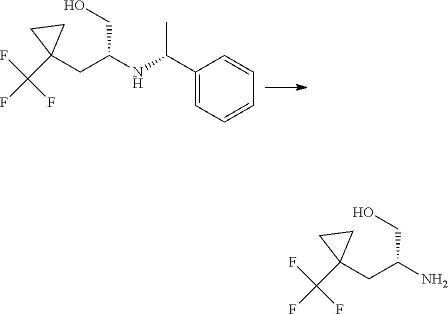 [SMILES: C[C@@H](N[C@@H](CO)CC1(C(F)(F)F)CC1)c1ccccc1 ; N[C@@H](CO)CC1(C(F)(F)F)CC1]

In a 1 L hydrogenation reactor, (2R)-2-[[(1R)-1-phenylethyl]amino]-3-[1-(trifluoromethyl)cyclopropyl]propan-1-ol (hydrochloride salt) (63.3 g, 195.5 mmol) was dissolved in EtOH (630 mL) (under warming), and it was treated with Pd/C (6.3 g of 10% w/w, 5.920 mmol) (12.5 g of 50% water wet) and the reaction was stirred under 2 bar of hydrogen at 40° C. for 24 h. The reaction mixture was filtered over Celite. The pad was washed with ethanol and the colorless filtrate was evaporated to a solid mass, which was triturated with diethyl ether. The suspension was stirred at room temperature for 1 h. The solid was filtered, washed with plenty of diethyl ether and dried to give (2R)-2-amino-3-[1-(trifluoromethyl)cyclopropyl]propan-1-ol (hydrochloride salt) (41.8 g, 97%) as an off-white solid. ¹H NMR (500 MHz, DMSO-d₆) δ 8.18 (s, 3H), 5.45 (t, J=4.9 Hz, 1H), 3.71 (dt, J 11.6, 3.9 Hz, 1H), 3.55 (dt, J=11.2, 5.4 Hz, 1H), 3.24 (h, J=4.7 Hz, 1H), 2.08 (dd, J=15.1, 5.4 Hz, 1H), 1.69 (dd, J=15.1, 9.4 Hz, 1H), 0.97 (h, J=6.5, 5.9 Hz, 2H), 0.86 (s, 2H). ESI-MS m/z calc. 183.0871, found 184.0 (M+1)⁺; Retention time: 0.65 minutes; LC method A.

### Step 8: 3-[[4-[(2R)-2-Amino-3-[1-(trifluoromethyl)cyclopropyl]propoxy]-6-(2,6-dimethylphenyl)pyrimidin-2-yl]sulfamoyl]benzoic acid

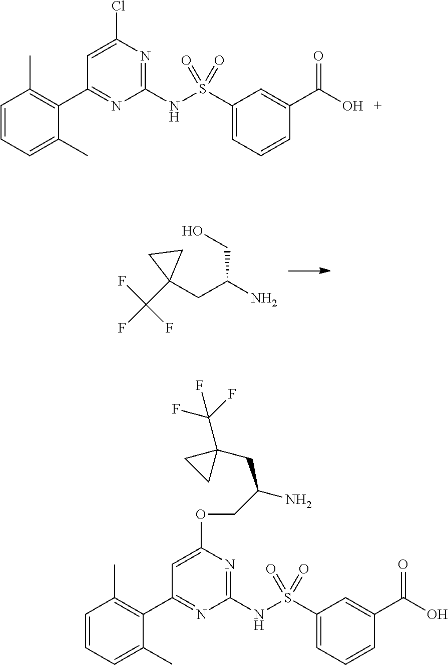

3-[[4-Chloro-6-(2,6-dimethylphenyl)pyrimidin-2-yl]sulfamoyl]benzoic acid (19.09 g, 45.68 mmol) and (2R)-2-amino-3-[1-(trifluoromethyl)cyclopropyl]propan-1-ol (hydrochloride salt) (10.18 g, 46.35 mmol) were dissolved in THE (100 mL) and cooled in an ice water bath. Sodium tert-butoxide (18.14 g, 188.8 mmol) was added and the reaction was allowed to warm to room temperature. The reaction was stirred for 1 h, then partitioned between ethyl acetate (500 mL) and aqueous HCl (275 mL of 1 M, 275.0 mmol). The organics were separated, washed with brine, dried over sodium sulfate and evaporated to give 3-[[4-[(2R)-2-amino-3-[1-(trifluoromethyl)cyclopropyl]propoxy]-6-(2,6-dimethylphenyl)pyrimidin-2-yl]sulfamoyl]benzoic acid (hydrochloride salt) (26.74 g, 94%). ESI-MS m/z calc. 564.1654, found 565.1 (M+1)⁺; Retention time: 0.48 minutes; LC method D.

### Example F: Preparation of (2R)-4-methyl-2-(spiro[2.3]hexan-5-ylamino)pentan-1-ol Step 1: (2R)-4-Methyl-2-(spiro[2.3]hexan-5-ylamino)pentan-1-ol

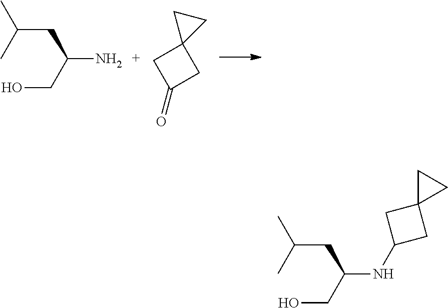

A mixture of spiro [2.3]hexan-5-one (100 g, 1.040 mol) and (2R)-2-amino-4-methyl-pentan-1-ol (123.5 g, 1.054 mol) in DCE (1.5 L) was stirred at ambient temperature for 1 h. To the mixture was added sodium triacetoxyborohydride (228 g, 1.076 mol) portionwise. The mixture was stirred at ambient temperature for 18 h. The reaction mixture was diluted with HCl (1.1 L of 2 M, 2.200 mol) until pH was ˜1. The aqueous phase was separated, and the organic phase extracted with HCl (600 mL of 2 M, 1.200 mol). The organic phase (DCE) was separated and the aqueous layer was basified with NaOH (550 g of 50% w/w, 6.875 mol) affording a solution at ˜ pH 12. The mixture was extracted 2× with EtOAc (1 L) and the combined organic phases were washed with brine (150 mL), dried over MgSO₄, filtered and concentrated in vacuo affording a clear oil. Used without further purification. (2R)-4-methyl-2-(spiro[2.3]hexan-5-ylamino)pentan-1-ol (160.7 g, 78%). ESI-MS m/z calc. 197.17796, found 198.2 (M+1)⁺; Retention time: 0.54 minutes (LC method A)

### Step 2: (2R)-4-Methyl-2-(spiro[2.3]hexan-5-ylamino)pentan-1-ol (hydrochloride salt)

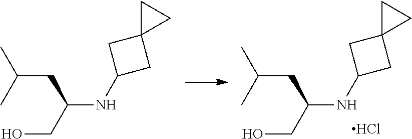

HCl (354 mL of 4 M, 1.416 mol) (4 M in dioxane) was added to a stirring (mechanical) solution of (2R)-4-methyl-2-(spiro[2.3]hexan-5-ylamino)pentan-1-ol (254 g, 1.287 mol) in diethyl ether (2.286 L) in an ice/ice water bath over 20 minutes, keeping the internal temp between 10° C. and 22° C. After the addition was complete, the solution was stirred at r.t. for 1.5 hours. The product was filtered out and rinsed with 2000 mL diethyl ether. The exact same process was repeated again on the exact same scale (a total of 508 g of amino alcohol SM was used). The product was dried under vacuum at 35° C. overnight and gave 562.3 g. (2R)-4-methyl-2-(spiro[2.3]hexan-5-ylamino)pentan-1-ol (Hydrochloride salt) (562.3 g, 93%). ¹H NMR (500 MHz, DMSO-d₆) δ 9.17-8.84 (m, 2H), 5.38 (s, 1H), 3.99 (p, J=7.2 Hz, 1H), 3.70-3.60 (m, 1H), 3.55-3.45 (m, 1H), 3.03-2.91 (m, 1H), 2.63-2.54 (m, 2H), 2.20-2.05 (m, 2H), 1.73-1.60 (m, 1H), 1.60-1.48 (m, 1H), 1.43-1.30 (m, 1H), 0.93-0.83 (m, 6H), 0.55-0.45 (m, 2H), 0.45-0.36 (m, 2H).

### Example G: Preparation of 3-[1-(trifluoromethyl)cyclopropyl]propan-1-ol

### Step 1: 2-[1-(Trifluoromethyl)cyclopropyl]ethyl methanesulfonate

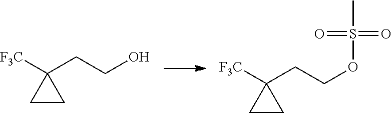

A 1000 mL, 3-neck round bottom flask was fitted with a mechanical stirrer, a cooling bath, a J-Kem temperature probe, an addition funnel and a nitrogen inlet/outlet. The vessel was charged under a nitrogen atmosphere with 2-[1-(trifluoromethyl)cyclopropyl]ethanol (125 g, 811.0 mmol) and 2-methyltetrahydrofuran (625 mL) which provided a clear colorless solution. Stirring was commenced and the pot temperature was recorded at 19° C. The vessel was then charged with triethylamine (124.3 mL, 891.8 mmol) added neat in one portion. The cooling bath was then charged with crushed ice/water and the pot temperature was lowered to 0° C. The addition funnel was charged with a solution of methanesulfonyl chloride (62.77 mL, 811.0 mmol) in 2-methyltetrahydrofuran (125 mL, 2 mL/g) which was subsequently added dropwise over 90 min which resulted in a white suspension and an exotherm to 1° C. The mixture was allowed to slowly warm to room temperature and continue to stir at room temperature for 1 h at which point the mixture was poured into ice cold water (250 mL) and then transferred to a separatory funnel. The organic was removed and washed with 20 wt % potassium bicarbonate solution (250 mL), dried over sodium sulfate (200 g) and then filtered through a glass frit Buchner funnel. The clear filtrate was concentrated under reduced pressure to provide 2-[1-(trifluoromethyl)cyclopropyl]ethyl methanesulfonate (185 g, 98%) as a clear pale yellow oil. ¹H NMR (400 MHz, Chloroform-d) δ 4.36 (ddt, J=7.1, 6.4, 0.7 Hz, 2H), 3.02 (s, 3H), 2.03 (t, J=7.1 Hz, 2H), 1.11-0.98 (m, 2H), 0.81-0.66 (m, 2H).

### Step 2: 3-[1-(Trifluoromethyl)cyclopropyl]propanenitrile

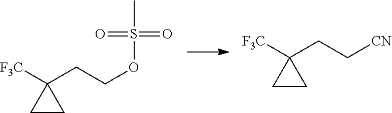

A 1000 mL, 3-nec round bottom as was fitted with a mechanical stirrer, a heating mantle, a J-Kem temperature probe/controller, a water cooled reflux condenser and a nitrogen inlet/outlet. The vessel was charged under a nitrogen atmosphere with 2-[1-(trifluoromethyl)cyclopropyl]ethyl methanesulfonate (50 g, 215.3 mmol) and dimethyl sulfoxide (250 mL) which provided a clear pale yellow solution. Stirring was commenced and the pot temperature was recorded at 19° C. The vessel was charged with sodium cyanide (13.19 g, 269.1 mmol), added as a solid in one portion. The mixture was heated to a pot temperature of 70° C. and the condition was maintained for 24 h. Upon heating all of the sodium cyanide dissolved and the reaction mixture turned to a light amber suspension. After cooling to room temperature, the reaction mixture was poured into water (500 mL) and then transferred to a separatory funnel and partitioned with methyl tert-butyl ether (500 mL). The organic was removed and the residual aqueous was extracted with methyl tert-butyl ether (3×250 mL). The combined organic layers were washed with water (2×250 mL), dried over sodium sulfate (200 g) and then filtered through a glass frit Buchner funnel. The clear filtrate was concentrated under reduced pressure to provide 3-[1-(trifluoromethyl)cyclopropyl]propanenitrile (30 g, 85%) as a clear amber oil. ¹H NMR (400 MHz, Chloroform-d) δ 2.55 (t, J=7.6 Hz, 2H), 1.93 (t, J=7.7 Hz, 2H), 1.11-1.04 (m, 2H), 0.78-0.70 (m, 2H).

### Step 3: 3-[1-(Trifluoromethyl)cyclopropyl]propanoic acid

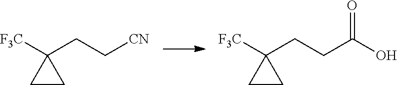 [SMILES: N#CCCC1(C(F)(F)F)CC1 ; O=C(O)CCC1(C(F)(F)F)CC1]

A 1000 mL, 3-neck round bottom flask was fitted with a mechanical stirrer, a heating mantle, a J-Kem temperature probe/controller, a water cooled reflux condenser and a nitrogen inlet/outlet. The vessel was subsequently charged under a nitrogen atmosphere with 3-[1-(trifluoromethyl)cyclopropyl]propanenitrile (25 g, 153.2 mmol) and ethyl alcohol (375 mL) which provided a clear amber solution. Stirring was commenced and the pot temperature was recorded at 19° C. The vessel was then charged with sodium hydroxide (102.1 mL of 6 M, 612.6 mmol), added in one portion. The resulting clear amber solution was heated to a pot temperature of 70° C. and the condition was maintained for 24 h. After cooling to room temperature, the reaction mixture was concentrated to remove the ethyl alcohol. The residual aqueous was diluted with water (150 mL) and then transferred to a separatory funnel and partitioned with methyl tert-butyl ether (50 mL). The aqueous was removed and the pH was adjusted to pH˜ 1 with 6 M hydrochloric acid solution. The resulting aqueous solution was transferred to a separatory funnel and partitioned with methyl tert-butyl ether (250 mL). The organic was removed and the residual aqueous was extracted with methyl tert-butyl ether (2×150 mL). The combined organic was dried over sodium sulfate (150 g) and then filtered through a glass frit Buchner funnel. The clear filtrate was concentrated under reduced pressure to provide 3-[1-(trifluoromethyl)cyclopropyl]propanoic acid (26 g, 93%) as a clear amber oil. ¹H NMR (400 MHz, Chloroform-d) δ 2.63-2.50 (m, 2H), 1.96-1.84 (m, 2H), 1.03-0.95 (m, 2H), 0.66-0.58 (m, J=1.7 Hz, 2H).

### Step 4: 3-[1-(Trifluoromethyl)cyclopropyl]propan-1-ol

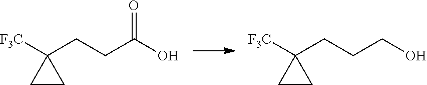 [SMILES: O=C(O)CCC1(C(F)(F)F)CC1 ; OCCCC1(C(F)(F)F)CC1]

A 1000 mL, 3-neck round bottom flask was fitted with a mechanical stirrer, a cooling bath, an addition funnel, a J-Kem temperature probe and a nitrogen inlet/outlet. The vessel was charged under a nitrogen atmosphere with lithium aluminum hydride pellets (6.775 g, 178.5 mmol). The vessel was then charged under a nitrogen atmosphere with tetrahydrofuran (250 mL). Stirring was commenced and the pot temperature was recorded at 20° C. The mixture was allowed to stir at room temperature for 0.5 h to allow the pellets to dissolve. The pot temperature of the resulting grey suspension was recorded at 24° C. The cooling bath was then charged with crushed ice/water and the pot temperature was lowered to 0° C. The addition funnel was charged with a solution of 3-[1-(trifluoromethyl)cyclopropyl]propanoic acid (25 g, 137.3 mmol) in tetrahydrofuran (75 mL, 3 mL/g) and the clear pale yellow solution was added dropwise over 1 h. After the addition was completed, the pot temperature of the resulting greyish-brown suspension was recorded at 5° C. The mixture was allowed to slowly warm to room temperature and continue to stir at room temperature for 24 h. The suspension was cooled to 0° C. with a crushed ice/water cooling bath and then quenched by the very slow dropwise addition of water (6.775 mL), followed by 15 wt % sodium hydroxide solution (6.775 mL) and then finally with water (20.32 mL). The pot temperature of the resulting white suspension was recorded at 5° C. The suspension was continued to stir at ˜5° C. for 30 min and then filtered through a glass frit Buchner funnel with a 20 mm layer of celite. The filter cake was displacement washed with tetrahydrofuran (2×150 mL) and then dried under vacuum for 15 min. The filtrate was dried over sodium sulfate (250 g) and then filtered through a glass frit Buchner funnel. The filtrate was concentrated under reduced pressure to provide a clear pale amber oil as the desired product, 3-[1-(trifluoromethyl)cyclopropyl]propan-1-ol (21.2 g, 92%). ¹H NMR (400 MHz, Chloroform-d) δ 3.65 (t, J=6.0 Hz, 2H), 1.78-1.59 (m, 4H), 0.99-0.91 (m, 2H), 0.59 (dp, J=4.7, 1.7 Hz, 2H).

### Example H: Preparation of 6-[[4-chloro-6-(2,6-dimethylphenyl)pyrimidin-2-yl]sulfamoyl]pyridine-2-carboxylic acid

### Step 1: Methyl 6-benzylsulfanylpyridine-2-carboxylate

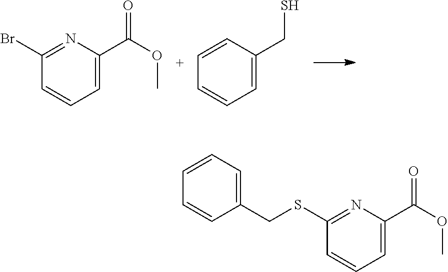

To a solution of phenylmethanethiol (28.408 g, 26.800 mL, 228.72 mmol) in THE (600 mL) was added NaH (11.200 g, 60% w/w, 280.03 mmol) in a few portions at 0° C. The slurry was warmed to room temperature and stirred for 30 min, then methyl 6-bromopyridine-2-carboxylate (50 g, 231.45 mmol) was added as a single portion. After 3 h, the reaction was diluted with ether (800 mL) and quenched with water (400 mL) and saturated sodium bicarbonate (50 mL). The layers were separated, and the organic layer was washed with brine, dried over sodium sulfate, and concentrated under reduced pressure to yield methyl 6-benzylsulfanylpyridine-2-carboxylate (56.35 g, 89%) as a yellow oil. ¹H NMR (500 MHz, DMSO-d₆) δ 7.84-7.77 (m, 1H), 7.77-7.73 (m, 1H), 7.52 (m, 1H), 7.48 (d, J=7.8 Hz, 2H), 7.28(t, J=7.2, 7.2 Hz, 2H), 7.24-7.18 (m, 1H), 4.44 (s, 2H), 3.90 (d, J=1.2 Hz, 3H). ESI-MS m/z calc. 259.0667, found 260.1 (M+1)⁺; Retention time: 3.2 minutes; LC method T.

### Step 2: Methyl 6-chlorosulfonylpyridine-2-carboxylate

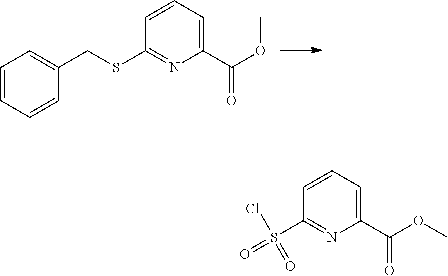 [SMILES: COC(=O)c1cccc(SCc2ccccc2)n1 ; COC(=O)c1cccc(S(=O)(=O)Cl)n1]

A solution of methyl 6-benzylsulfanylpyridine-2-carboxylate (121.62 g, mmol) in DCM (950 mL) and DI water (300 mL) was cooled in a −1-0° C. ice bath and, with vigorous stirring, sulfuryl chloride (228.14 g, 140 mL, 1.6396 mol) was added dropwise while the temperature was maintained below 5° C. After the addition, the organic phase was separated, washed with DI water (2×500 mL), dried over anhydrous sodium sulfate, filtered and concentrated under vacuum. The residue was dissolved in DCM (500 mL). Hexanes (1000 mL) was added and the DCM was slowly evaporated off. The white precipitate was filtered by vacuum and the solids were washed with Hexanes (2×500 mL). The filtered solids were collected. The residue solids in the filtrate were filtered and dissolved in DCM (500 mL). The DCM solution was transferred to a 1 L round-bottom flask and concentrated under vacuum. The residue was dissolved in DCM (200 mL). Hexanes (600 mL) was added and the DCM was slowly evaporated off. The white precipitation was filtered by vacuum and the solids were washed with hexanes (2×500 mL) After drying, methyl 6-chlorosulfonylpyridine-2-carboxylate (56.898 g, 55%) was isolated. ¹H NMR (500 MHz, Chloroform-d) δ 8.48 (dd, J=7.8, 1.1 Hz, 1H), 8.31 (dd, J=7.9, 1.1 Hz, 1H), 8.25 (t, J=7.8 Hz, 1H), 4.08 (s, 3H). ESI-MS m/z calc. 234.97061, found 236.1 (M+1)⁺; Retention time: 1.74 minutes; LC method T.

### Step 3: Methyl 6-[[4-chloro-6-(2,6-dimethylphenyl)pyrimidin-2-yl]sulfamoyl]pyridine-2-carboxylate

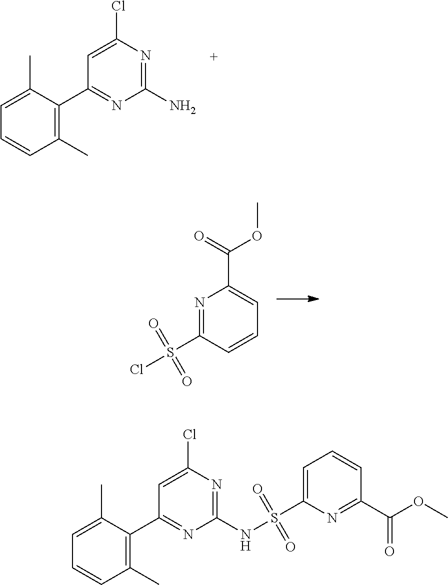 [SMILES: Cc1cccc(C)c1-c1cc(Cl)nc(N)n1 ; COC(=O)c1cccc(S(=O)(=O)Cl)n1 ; COC(=O)c1cccc(S(=O)(=O)Nc2nc(Cl)cc(-c3c(C)cccc3C)n2)n1]

A solution of 4-chloro-6-(2,6-dimethylphenyl)pyrimidin-2-amine (16.63 g, 71.161 mmol) and methyl 6-chlorosulfonylpyridine-2-carboxylate (16.8 g, 71.294 mmol) dissolved in anhydrous THE (680 mL) was cooled to −78° C. Then Lithium bis(trimethylsilyl)amide (143 mL of 1 M, 143.00 mmol) in solution in THE was added dropwise. The mixture was allowed to warm up to 0° C. slowly and then 1M aqueous HCl (146 mL) was added, followed by DI water (680 mL). The THF was evaporated and the aqueous phase was extracted with chloroform (3×250 mL). The combined organic layers were washed with saturated aqueous NaCl (300 mL), dried over anhydrous sodium sulfate, filtered, and concentrated under vacuum. The crude was recrystallized in 10% Acetone in Hexanes (500 mL). The white precipitate was filtered and rinsed with acetone (2×100 mL) to give methyl 6-[[4-chloro-6-(2,6-dimethylphenyl)pyrimidin-2-yl]sulfamoyl]pyridine-2-carboxylate (15.79 g, 50%). ESI-MS m/z calc. 432.06592, found 433.3 (M+1)⁺; Retention time: 5.5 minutes; LC method S.

### Step 4: 6-[[4-Chloro-6-(2,6-dimethylphenyl)pyrimidin-2-yl]sulfamoyl]pyridine-2-carboxylic acid

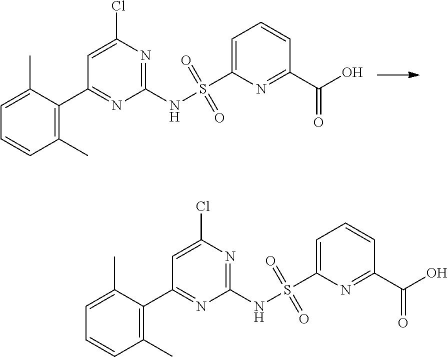 [SMILES: Cc1cccc(C)c1-c1cc(Cl)nc(NS(=O)(=O)c2cccc(C(=O)O)n2)n1 ; Cc1cccc(C)c1-c1cc(Cl)nc(NS(=O)(=O)c2cccc(C(=O)O)n2)n1]

To a solution of methyl 6-[[4-chloro-6-(2,6-dimethylphenyl)pyrimidin-2 yl]sulfamoyl]pyridine-2-carboxylate (15.79 g, 36.477 mmol) in THE (180 mL) was added aqueous sodium hydroxide (182 mL of 1 M, 182.00 mmol). The reaction was stirred at RT for 1 h. The THF was evaporated, and the aqueous layer was washed with diethyl ether (2×200 mL). The aqueous layer was acidified to pH 2 with 1 M Aqueous HCl (250 mL). The precipitate was filtered and the a white solid were rinsed with DI water (2×250 mL). The solids were dried under vacuum to give 6-[[4-chloro-6-(2,6-dimethylphenyl)pyrimidin-2-yl]sulfamoyl]pyridine-2-carboxylic acid (14.3444 g, 93%). ¹H NMR (250 MHz, DMSO-d₆) δ 8.14-7.99 (m, 3H), 7.21-7.11 (m, 1H), 7.03 (d, J=7.7 Hz, 2H), 6.92 (s, 1H), 1.78 (s, 6H). ESI-MS m/z calc. 418.05026, found 419.1 (M+1)⁺; Retention time: 2.61 minutes; LC method T.

### Example I: Preparation of 3-[[4-[(2R)-2-Amino-4-methyl-pentoxy]-6-(2,6-dimethylphenyl)-2-pyridyl]sulfamoyl]benzoic acid

### Step 1: 4-Chloro-6-(26-dimethylphenyl)pyridin-2-amine

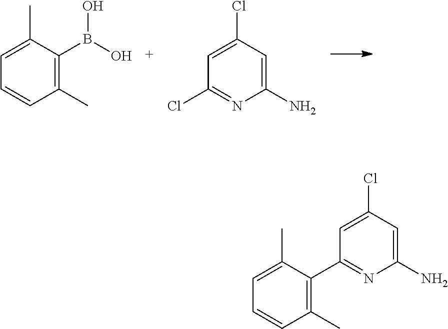 [SMILES: Cc1cccc(C)c1B(O)O ; Nc1cc(Cl)cc(Cl)n1 ; Cc1cccc(C)c1-c1cc(Cl)cc(N)n1]

To a stirring solution of (2,6-dimethylphenyl)boronic acid (11.515 g, 76.775 mmol) and 4,6-dichloropyridin-2-amine (12.513 g, 76.765 mmol) in Toluene (425 mL) and EtOH (213 mL) was added an aqueous solution of Sodium carbonate (115 mL of 2 M, 230.00 mmol) and the reaction mixture was degassed with nitrogen gas for 45 min. Pd(dppf)Cl₂ (6.271 g, 7.6791 mmol) was then added with degassing continuing for an additional 15 min. Then the reaction vial was sealed, and the mixture heated to 100° C. and stirred at that temperature for 24 h. After this time, volatiles were removed under reduced pressure and the residue was extracted with ethyl acetate (3×200 mL). The combined organic layers were washed with brine, dried over anhydrous sodium sulfate and concentrated under reduced pressure. The crude product was purified by silica gel column chromatography (0-25% EtOAc in Hexanes) and triturated with Hexanes to afford 4-chloro-6-(2,6-dimethylphenyl)pyridin-2-amine (6.469 g, 34%) as an off-white solid. ESI-MS m/z calc. 232.07672, found 233.1 (M+1)⁺; Retention time: 2.31 minutes; (LC method T).

### Step 2: Methyl 3-[[4-chloro-6-(2,6-dimethylphenyl)-2-pyridyl]sulfamoyl]benzoate

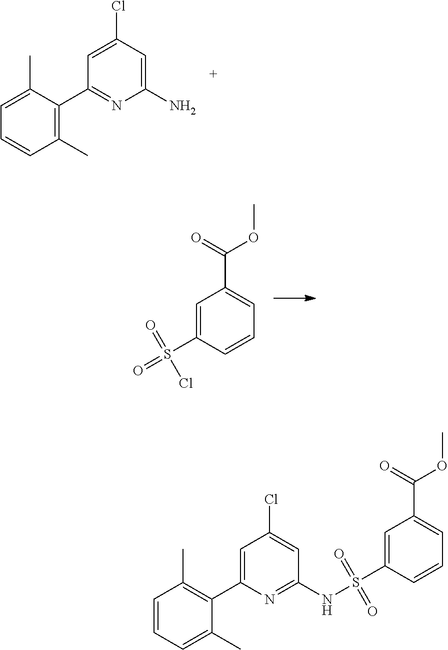 [SMILES: Cc1cccc(C)c1-c1cc(Cl)cc(N)n1 ; COC(=O)c1cccc(S(=O)(=O)Cl)c1 ; COC(=O)c1cccc(S(=O)(=O)Nc2cc(Cl)cc(-c3c(C)cccc3C)n2)c1]

To a solution of 4-chloro-6-(2,6-dimethylphenyl)pyridin-2-amine (4.9 g, 20.635 mmol) and methyl 3-chlorosulfonylbenzoate (4.9 g, 20.046 mmol) in THE (200 mL) was added dropwise Lithium bis(trimethylsilyl)amide (45 mL of 1 M, 45.000 mmol) at −78° C. under nitrogen. The reaction mixture was stirred for 30 minutes at −78° C.; then warmed up to 0° C. and stirred for 2 hours at 0° C. The reaction was quenched with cold 1.0 M Hydrochloric acid (50 mL) and diluted with water (200 mL). The mixture was extracted with ethyl acetate (2×400 mL). The organic layers were combined, washed with brine (500 mL), dried over sodium sulfate, filtered and concentrated. The residue was purified by chromatography using 0-20% ethyl acetate in hexanes to afford methyl 3-[[4-chloro-6-(2,6-dimethylphenyl)-2-pyridyl]sulfamoyl]benzoate (6.2 g, 68%) as a white solid. ESI-MS m/z calc. 430.0754, found 431.5 (M+1)⁺; Retention time: 3.65 minutes; (LC method T).

### Step 3: 3-[[4-chloro-6-(2,6-dimethylphenyl)-2-pyridyl]sulfamoyl]benzoic acid

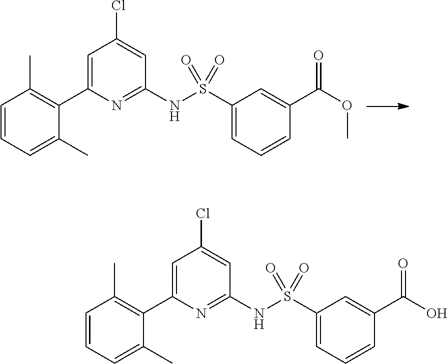 [SMILES: COC(=O)c1cccc(S(=O)(=O)Nc2cc(Cl)cc(-c3c(C)cccc3C)n2)c1 ; Cc1cccc(C)c1-c1cc(Cl)cc(NS(=O)(=O)c2cccc(C(=O)O)c2)n1]

To a stirring solution of 3-[4-chloro-6-(2,6-dimethyl-phenyl)-pyridin-2-ylsulfamoyl]-benzoic acid methyl ester (5.3 g, 12.3 mmol) in a mixture of tetrahydrofuran (80 mL) and water (80 mL) at room temperature was added lithium hydroxide monohydrate (1.55 g, 36.9 mmol) and the reaction mixture was stirred at 45 C for 2 hours. Tetrahydrofuran was removed under vacuum and the residue was diluted with water (100 mL). The aqueous layer was washed with diethyl ether (2×50 mL), hexanes (50 mL) and acidified with 1.0 M hydrochloric acid to pH=2-3. The precipitated product was collected by filtration and dried in a vacuum oven at 75° C. to constant weight to afford 3-[4-chloro-6-(2,6-dimethyl-phenyl)-pyridin-2-ylsulfamoyl]-benzoic acid (4.8 g, 93%) as a white solid. ¹H NMR (250 MHz, DMSO-d₆) δ (ppm): 8.32 (d, J=1.9 Hz, 1H), 8.14 (d, J=7.7 Hz, 1H), 8.03 (d, J=8.0 Hz, 1H), 7.63 (t, J=7.8 Hz, 1H), 7.28-6.96 (m, 5H), 1.77 (s, 6H). ESI-MS m/z calc. 416.8, found 417.0 (M1). Retention time: 5.11 minutes.

### Step 4: 3-[[4-[(2R)-2-Amino-4-methyl-pentoxy]-6-(2,6-dimethylphenyl)-2-pyridyl]sulfamoyl]benzoic acid

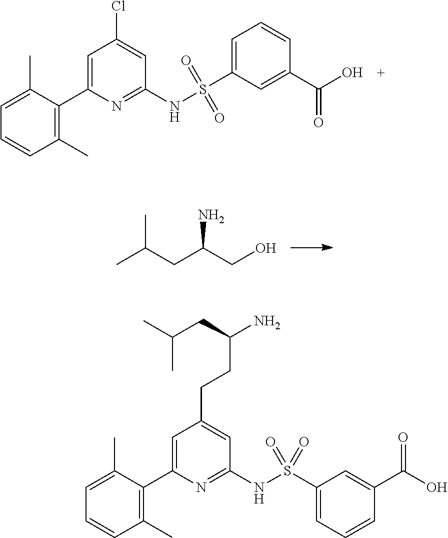

A 20 mL vial was charged with 3-[[4-chloro-6-(2,6-dimethylphenyl)-2-pyridyl]sulfamoyl]benzoic acid (300 mg, 0.7196 mmol), (2R)-2-amino-4-methyl-pentan-1-ol (110 mg, 0.9387 mmol) and anhydrous tetrahydrofuran (12 mL), in that order. Then the vial was purged with nitrogen for 30 seconds, and solid potassium tert-butoxide (350 mg, 3.119 mmol) was added capped under nitrogen. After stirred at 105° C. for 14 h (overnight), the reaction was allowed to cool to ambient temperature. Then glacial acetic acid (200 μL, 3.517 mmol) was added and the volatiles were removed under reduced pressure. To the residue, DMSO (5 mL) was added and microfiltered. Purification by reverse phase chromatography (Cis column, 1-99% acetonitrile in water over 15 min) gave 3-[[4-[(2R)-2-amino-4-methyl-pentoxy]-6-(2,6-dimethylphenyl)-2-pyridyl]sulfamoyl]benzoic acid (hydrochloride salt)(278 mg, 72%) as yellowish solid. ESI-MS m/z calc. 497.19846, found 498.2 (M+1)⁺; Retention time: 0.43 minutes (LC method D).

### Example J: Preparation of methyl 3-[[4-chloro-6-(2,6-dimethylphenyl)pyrimidin-2-yl]-(methoxymethyl)sulfamoyl]benzoate

### Step 1: Methyl 3-[[4-chloro-6-(2,6-dimethylphenyl)pyrimidin-2-yl]-(methoxymethyl)sulfamoyl]benzoate

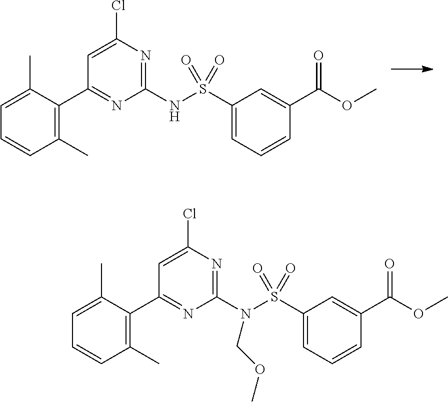 [SMILES: COC(=O)c1cccc(S(=O)(=O)Nc2nc(Cl)cc(-c3c(C)cccc3C)n2)c1 ; COCN(c1nc(Cl)cc(-c2c(C)cccc2C)n1)S(=O)(=O)c1cccc(C(=O)OC)c1]

To a solution of methyl 3-[[4-chloro-6-(2,6-dimethylphenyl)pyrimidin-2-yl]sulfamoyl]benzoate (35.04 g, 81.131 mmol) in Acetonitrile (525 mL) and 1,2-dichloroethane (525 mL) was added potassium carbonate (16.8 g, 121.56 mmol) followed by Chloromethyl methyl ether (7.5260 g, 7.1 mL, 93.475 mmol). The reaction mixture was stirred at room temperature for overnight. The solvent was evaporated, and the resulting material was partitioned between water (300 mL) and EtOAc (300 mL). The aqueous layer was extracted with EtOAc (2×200 mL). The combined organic layers were washed with water (300 mL) and brine (300 mL), dried over anhydrous sodium sulfate and concentrated under vacuum. The residue was purified by silica gel chromatography using 0 to 40% EtOAc in Hexane to afford methyl 3-[[4-chloro-6-(2,6-dimethylphenyl)pyrimidin-2-yl]-(methoxymethyl)sulfamoyl]benzoate (30.95 g, 80%) as clear jell. ESI-MS m/z calc. 475.0969, found 476.3 (M+1)⁺; Retention time: 3.96 minutes, LC method T.

### Example K: Preparation of 3-[[4-(2-amino-4,4,4-trifluoro-butoxy)-6-(2,6-dimethylphenyl)pyrimidin-2-yl]sulfamoyl]benzoic acid

### Step 1: 3-[[4-[2-(tert-Butoxycarbonylamino)-4,4,4-trifluoro-butoxy]-6-(2,6-dimethylphenyl)pyrimidin-2-yl]sulfamoyl]benzoic acid

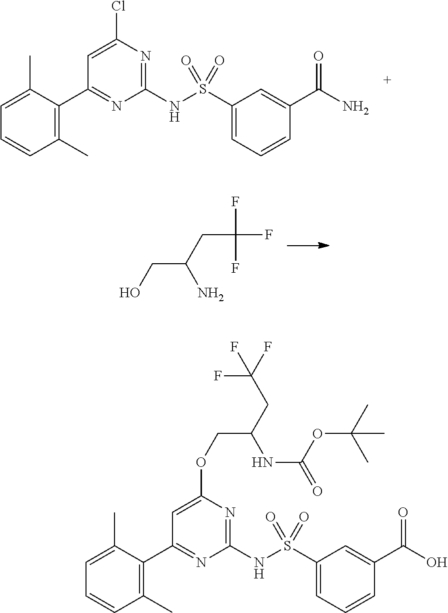 [SMILES: Cc1cccc(C)c1-c1cc(Cl)nc(NS(=O)(=O)c2cccc(C(N)=O)c2)n1 ; NC(CO)CC(F)(F)F ; Cc1cccc(C)c1-c1cc(OCC(CC(F)(F)F)NC(=O)OC(C)(C)C)nc(NS(=O)(=O)c2cccc(C(=O)O)c2)n1]

A solution of 3-[[4-chloro-6-(2,6-dimethylphenyl)pyrimidin-2-yl]sulfamoyl]benzoic acid (0.63 g, 1.508 mmol), 2-amino-4,4,4-trifluoro-butan-1-ol (hydrochloride salt) (0.54 g, 3.007 mmol), and sodium t-butoxide (0.73 g, 7.596 mmol) in THF (8 mL) was stirred for five minutes, turning bright yellow. The reaction was placed in a preheated 60° C. bath and stirred for 25 minutes. UPLCMS showed complete conversion to amino intermediate. After cooling to room temperature, di-tert-butyl dicarbonate (0.67 g, 3.070 mmol) was added, and the reaction was stirred for 17 hours. The reaction was quenched with 1 M hydrochloric acid, diluted with water, and extracted with ethyl acetate. The combined extracts were washed with water, dried over sodium sulfate, and evaporated under vacuum. The residue was purified by silica gel column chromatography with 0-10% methanol in dichloromethane to give a mixture containing product. The mixture was re-purified by silica gel column chromatography with 0-9% methanol in dichloromethane to give 3-[[4-[2-(tert-butoxycarbonylamino)-4,4,4-trifluoro-butoxy]-6-(2,6-dimethylphenyl)pyrimidin-2-yl]sulfamoyl]benzoic acid (0.54 g, 57%) ESI-MS m/z calc. 624.1866, found 625.3 (M+1)⁺; Retention time: 0.67 minutes as a colorless solid, LC method D.

### Step 2: 3-[[4-(2-Amino-4,4,4-trifluoro-butoxy)-6-(2,6-dimethylphenyl)pyrimidin-2-yl]sulfamoyl]benzoic acid

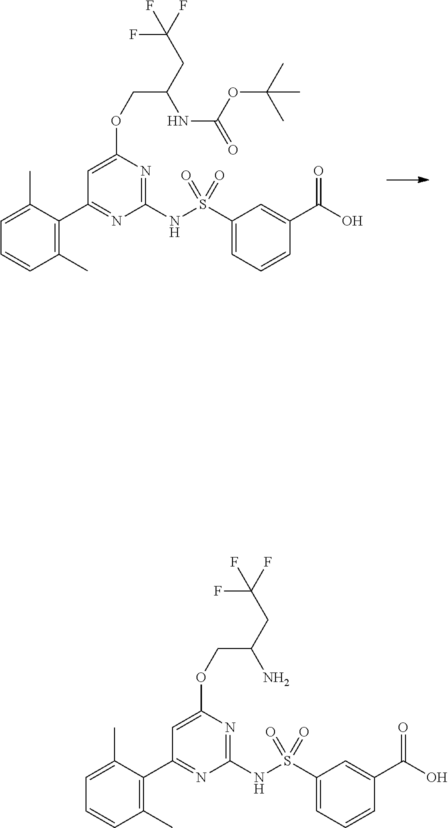 [SMILES: Cc1cccc(C)c1-c1cc(OCC(CC(F)(F)F)NC(=O)OC(C)(C)C)nc(NS(=O)(=O)c2cccc(C(=O)O)c2)n1 ; Cc1cccc(C)c1-c1cc(OCC(N)CC(F)(F)F)nc(NS(=O)(=O)c2cccc(C(=O)O)c2)n1]

A solution of 3-[[4-[2-(tert-butoxycarbonylamino)-4,4,4-trifluoro-butoxy]-6-(2,6-dimethylphenyl)pyrimidin-2-yl]sulfamoyl]benzoic acid (83 mg, 0.1329 mmol) and HCl (4 mL of 4 M, 16.00 mmol) (in dioxane) was stirred for one hour. The solvent was removed under vacuum, and the solids were triturated with diethyl ether to give 3-[[4-(2-amino-4,4,4-trifluoro-butoxy)-6-(2,6-dimethylphenyl)pyrimidin-2-yl]sulfamoyl]benzoic acid (hydrochloride salt) (81 mg, 109%) ESI-MS m/z calc. 524.13416, found 525.2 (M+1)⁺; Retention time: 0.39 minutes as a colorless solid, LC method D.

### Example L: Preparation of 3-[[4-[(2R)-2-aminopropoxy]-6-(2,6-dimethylphenyl)pyrimidin-2-yl]sulfamoyl]benzoic acid

### Step 1: 3-[[4-[(2R)-2-(tert-Butoxycarbonylamino)propoxy]-6-(2,6-dimethylphenyl)pyrimidin-2-yl]sulfamoyl]benzoic acid

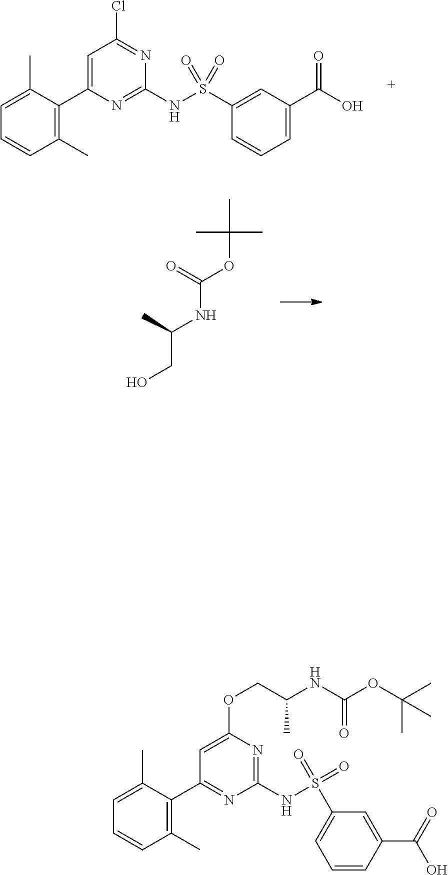 [SMILES: Cc1cccc(C)c1-c1cc(Cl)nc(NS(=O)(=O)c2cccc(C(=O)O)c2)n1 ; C[C@H](CO)NC(=O)OC(C)(C)C ; Cc1cccc(C)c1-c1cc(OC[C@@H](C)NC(=O)OC(C)(C)C)nc(NS(=O)(=O)c2cccc(C(=O)O)c2)n1]

A solution of 3-[[4-chloro-6-(2,6-dimethylphenyl)pyrimidin-2-yl]sulfamoyl]benzoic acid (75 mg, 0.1795 mmol) in THE (0.7 mL) was added to tert-butyl N-[(1R)-2-hydroxy-1-methyl-ethyl]carbamate (approximately 47.17 mg, 0.2692 mmol). Solid sodium tert-butoxide (approximately 86.25 mg, 0.8975 mmol) was added after. The reaction mixture was allowed to stir overnight at room temperature. Acetic acid (approximately 64.68 mg, 61.25 μL, 1.077 mmol) was added. The reaction mixture was diluted with DCM and washed with HCl (1 M, 1×7 mL) and brine (2×75 mL). The organic layer was dried over sodium sulfate, filtered and concentrated under reduced pressure. The crude product was chromatographed on a 12 gram silica gel column eluting with a EtOAc/hexane gradient. 3-[[4-[(2R)-2-(tert-Butoxycarbonylamino)propoxy]-6-(2,6-dimethylphenyl)pyrimidin-2-yl]sulfamoyl]benzoic acid (1.65 g, 2.964 mmol) (65 mg, 65%) was obtained. ESI-MS m/z calc. 556.19916, found 557.3 (M+1)⁺; Retention time: 1.63 minutes; LC method A.

### Step 2: 3-[[4-[(2R)-2-Aminopropoxy]-6-(2,6-dimethylphenyl)pyrimidin-2-yl]sulfamoyl]benzoic acid

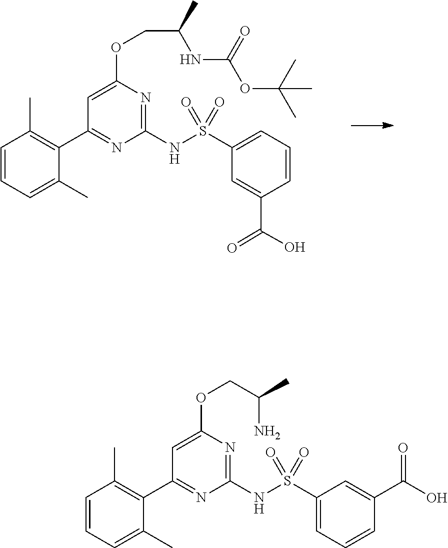 [SMILES: Cc1cccc(C)c1-c1cc(OC[C@@H](C)NC(=O)OC(C)(C)C)nc(NS(=O)(=O)c2cccc(C(=O)O)c2)n1 ; Cc1cccc(C)c1-c1cc(OC[C@@H](C)N)nc(NS(=O)(=O)c2cccc(C(=O)O)c2)n1]

A solution of 3-[[4-[(2R)-2-(tert-butoxycarbonylamino)propoxy]-6-(2,6-dimethylphenyl)pyrimidin-2-yl]sulfamoyl]benzoic acid (1.65 g, 2.964 mmol) in HCl (8 mL of 4 M, 32.00 mmol) (in dioxane) was stirred for two hours, and the solvent was removed under vacuum. The solids were triturated with diethyl ether and dried under vacuum to give 3-[[4-[(2R)-2-aminopropoxy]-6-(2,6-dimethylphenyl)pyrimidin-2-yl]sulfamoyl]benzoic acid (hydrochloride salt) (1.55 g, 106%) as a colorless solid. ESI-MS m/z calc. 456.14673, found 457.2 (M+1)⁺; Retention time: 0.37 minutes, LC method D.

### Example M: Preparation of 3-[[4-[(2R)-2-amino-5-hydroxy-5-methyl-hexoxy]-6-(2,6-dimethylphenyl)pyrimidin-2-yl]sulfamoyl]benzoic acid

### Step 1: Benzyl (4R)-4-(tert-butoxycarbonylamino)-5-hydroxy-pentanoate

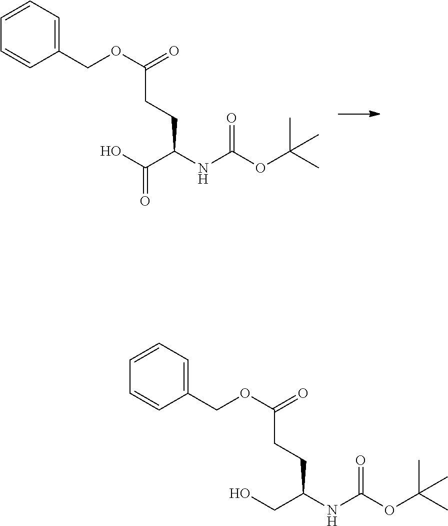 [SMILES: CC(C)(C)OC(=O)N[C@H](CCC(=O)OCc1ccccc1)C(=O)O ; CC(C)(C)OC(=O)N[C@@H](CO)CCC(=O)OCc1ccccc1]

(2R)-5-Benzyloxy-2-(tert-butoxycarbonylamino)-5-oxo-pentanoic acid (10 g, 29.641 mmol) was dissolved in dimethoxyethane (30 mL) and the solution was cooled to −15° C. N-methylmorpholine (3.0360 g, 3.3 mL, 30.016 mmol) was added followed by a slow addition of isobutyl chloroformate (4.1067 g, 3.9 mL, 30.069 mmol) such that the reaction temperature was kept below −10° C. The mixture was stirred for 30 minutes. The solids were quickly filtered and washed with dimethoxyethane (30 mL). The filtrate was cooled to −40° C. and a solution of sodium borohydride (1.45 g, 38.327 mmol) in water (15 mL) was added slowly such that the reaction temperature was maintained between −30° C. and −15° C. The mixture was stirred for 15 minutes. Water (180 mL) was then added dropwise at −15° C. and the temperature was slowly raised to 5° C. while controlling the gas evolution. The suspension was filtered and washed with water (300 mL). The solid was dissolved in dichloromethane (100 mL) and transferred in a separatory funnel. Phases were separated, the organic phase was dried over sodium sulfate, filtered and evaporated to dryness to give benzyl (4R)-4-(tert-butoxycarbonylamino)-5-hydroxy-pentanoate (7.98 g, 83%) as a white solid. ¹H NMR (400 MHz, CDCl₃) δ 7.42-7.30 (m, 5H), 5.13 (s, 2H), 4.81 (br. s., 1H), 3.65 (br. s., 2H), 3.60-3.51 (m, 1H), 2.57-2.36 (m, 3H), 1.98-1.87 (m, 1H), 1.86-1.73 (m, 1H), 1.44 (s, 9H). ESI-MS m/z calc. 323.1733, found 224.4 (M-99)⁺; Retention time: 1.696 minutes, LC method X.

### Step 2: Benzyl 3-[(4R)-2-oxooxazolidin-4-yl]propanoate

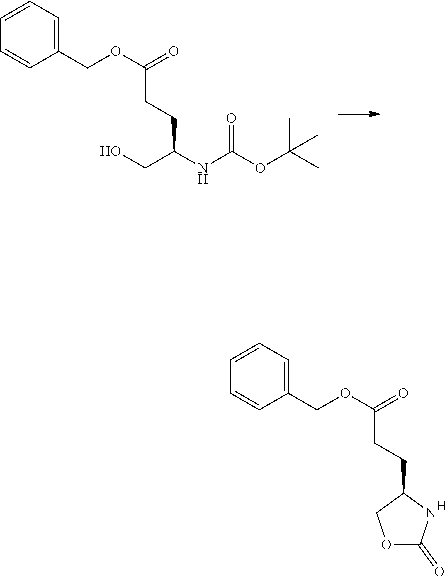

To a solution of benzyl (4R)-4-(tert-butoxycarbonylamino)-5-hydroxy-pentanoate (7.98 g, 24.652 mmol) in dichloroethane (80 mL) was added pyridine (48.900 g, 50 mL, 618.21 mmol). p-toluenesulfonic anhydride (8.65 g, 25.972 mmol) was then added and the mixture was stirred at room temperature for 1 hour and then heated to 90° C. for 2 hours. The mixture was cooled, diluted with dichloromethane (150 mL) and washed with 1N HCl (3×100 mL). The combined organic layers were washed with brine, dried with sodium sulfate and the solvents were removed in vacuo. The residue was purified by silica-gel column chromatography on a 80 g column, eluting from 20% to 80% of EtOAc in heptane to yield benzyl 3-[(4R)-2-oxooxazolidin-4-yl]propanoate (4.85 g, 77%) as a pale brown oil that slowly crystalized over time. ¹H NMR (400 MHz, CDCl₃) δ 7.43-7.30 (m, 5H), 6.15 (br. s., 1H), 5.13 (s, 2H), 4.48 (t, J=8.4 Hz, 1H), 4.02 (dd, J=8.6, 6.1 Hz, 1H), 3.97-3.88 (m, 1H), 2.45 (t, J=7.3 Hz, 2H), 2.00-1.85 (m, 2H). ESI-MS m/z calc. 249.1001, found 250.2 (M+1)⁺; Retention time: 1.511 minutes, LC method X.

### Step 3: (4R)-4-(3-Hydroxy-3-methyl-butyl)oxazolidin-2-one

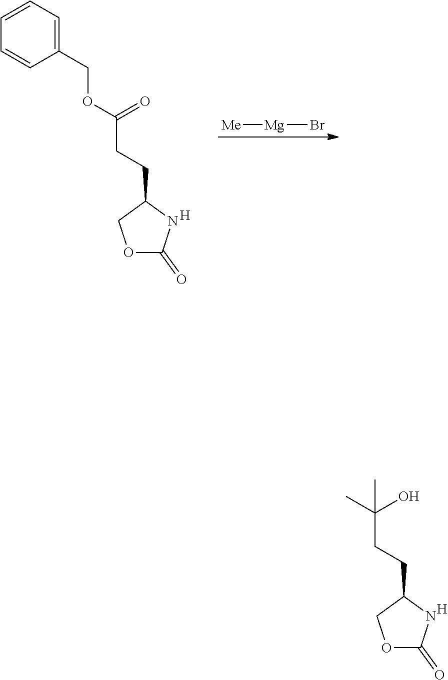

Methylmagnesium bromide (26 mL of 3 M, 78.000 mmol) in diethyl ether was added to a mixture of toluene (42 mL) and tetrahydrofuran (42 mL) at −20° C. (methanol+water+dried ice). A warm tetrahydrofuran (22 mL) solution of benzyl 3-[(4R)-2-oxooxazolidin-4-yl]propanoate (4.85 g, 19.457 mmol) was then added dropwise maintaining the temperature below −10° C. The mixture was warmed up to room temperature and stirred for 2 hours. The reaction mixture was cooled to 0° C., quenched with a 10% aqueous acetic acid solution (50 mL) and the resultant mixture was stirred for 1 hour at room temperature. The layers were separated. The aqueous layer was extracted with methyl-THF (3×100 mL) and then with dichloromethane (2×100 mL). The organic phases were combined, dried on anhydrous sodium sulfate, filtered and concentrated under reduced pressure. The residue was purified by silica-gel column chromatography on a 50 g and 120 g column, eluting from 0 to 15% of isopropanol in dichloromethane to afford (4R)-4-(3-hydroxy-3-methyl-butyl)oxazolidin-2-one (1.73 g, 51%) as a white solid. ¹H NMR (400 MHz, CDCl₃) δ 6.05 (br. s., 1H), 4.50 (t, J=8.4 Hz, 1H), 4.03 (dd, J=8.4, 6.2 Hz, 1H), 3.95-3.81 (m, 1H), 1.76-1.64 (m, 2H), 1.59-1.44 (m, 3H), 1.25 (s, 6H). ESI-MS m/z calc. 173.1052, found 174.2 (M+1)⁺; Retention time: 0.95 minutes, LC method X.

### Step 4: (2R)-2-Amino-5-methyl-hexane-1,5-diol

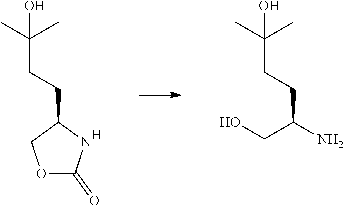

A mixture of (4R)-4-(3-hydroxy-3-methyl-butyl)oxazolidin-2-one (307 mg, 1.7724 mmol), barium hydroxide octahydrate (1.69 g, 5.3572 mmol), ethanol (12 mL) and water (12 mL) was heated at 95° C. to reflux for 2 hours. Reaction mixture was cooled to room temperature before dry ice was slowly added (˜1.8 g) and mixture was stirred vigorously for 2 days. The suspension was filtered over a Celite pad and rinsed with ethanol (˜15 mL). The filtrate was diluted with toluene, co-evaporated three times and concentrated under reduced pressure. Barium salts were observed on the walls of the flask. A minimum of ethanol was added, and the solution was filtered a second time over a Celite pad. The filtrate was concentrated under pressure to provide (2R)-2-amino-5-methyl-hexane-1,5-diol (338.4 mg, 130%) as a yellow oil. The crude was used for the next step without purification. ¹H NMR (400 MHz, DMSO-d₆) δ 3.40-3.28 (m, 1H), 3.25-3.11 (m, 1H), 2.64 (br. s, 1H), 1.81 (s, 2H), 1.51-1.37 (m, 2H), 1.37-1.29 (m, 1H), 1.29-1.18 (m, 1H), 1.06 (d, J=1.0 Hz, 6H). ESI-MS m/z calc. 147.1259, found 148.4 (M+1)⁺; Retention time: 0.22 minutes, LC method X.

### Step 5: 3-[[4-[(2R)-2-Amino-5-hydroxy-5-methyl-hexoxy]-6-(2,6-dimethylphenyl)pyrimidin-2-yl]sulfamoyl]benzoic acid

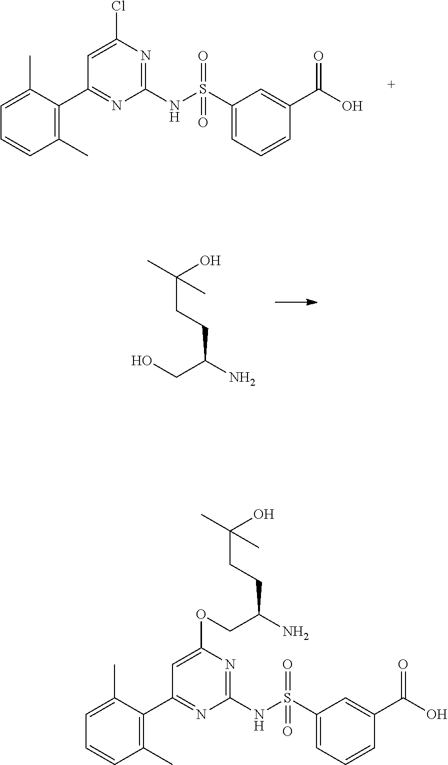

To a solution of 3-[[4-chloro-6-(2,6-dimethylphenyl)pyrimidin-2-yl]sulfamoyl]benzoic acid (371 mg, 0.8878 mmol) and (2R)-2-amino-5-methyl-hexane-1,5-diol (261 mg, 1.7729 mmol) in THE cooled down to 0° C. was slowly added Sodium tert-butoxide (375 mg, 3.9020 mmol). After 2 hours sodium tert-butoxide (76 mg, 0.7908 mmol) was slowly added to the reaction and stirred at room temperature. After 2 hours following the addition, sodium tert-butoxide in THE (200 μL of 2 M, 0.4000 mmol) was slowly added and the reaction was stirred at room temperature overnight. The reaction was partitioned between ethyl acetate (6 mL) and hydrochloric acid 1N (6 mL). The aqueous phase was extracted with ethyl acetate (2×6 mL) and 2-methyltetrahydrofuran (3×6 mL). The organic phases were combined, dried over sodium sulfate, filtered and concentrated to dryness. The solid was triturated with ethyl acetate (10 mL) and the precipitate was filtered then washed with ethyl acetate (2×10 mL) to afford 3-[[4-[(2R)-2-amino-5-hydroxy-5-methyl-hexoxy]-6-(2,6-dimethylphenyl)pyrimidin-2-yl]sulfamoyl]benzoic acid (653.4 mg, 139%, higher mass recovery might be due to salt contamination) as a pale-yellow solid. The crude was used for the next step without purification. ¹H NMR (400 MHz, DMSO-d₆) δ 13.24 (br. s, 1H), 8.43 (s, 1H), 8.19-8.06 (m, 3H), 7.70 (t, J=7.6 Hz, 1H), 7.32-7.19 (m, 1H), 7.18-7.05 (m, 2H), 6.30 (s, 1H), 4.46-4.32 (m, 1H), 4.30-4.18 (m, 1H), 3.53 (s, 1H), 1.99 (s, 6H), 1.78-1.61 (m, 2H), 1.57-1.37 (m, 2H), 1.11 (d, J 7.8 Hz,6H). ESI-MS m/z calc. 528.2043, found 529.2 (M+1)⁺; Retention time: 1.3 minutes, LC method X.

### Example N: Preparation of 3-[[4-[(2R)-2-amino-4-methyl-pentoxy]-6-[2-(benzyloxymethyl)-6-methyl-phenyl]pyrimidin-2-yl]sulfamoyl]benzoic acid

### Step 1: (2-Bromo-3-methyl-phenyl)methanol

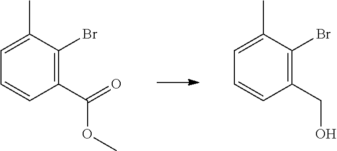

To a solution of methyl 2-bromo-3-methyl-benzoate (10.0281 g, 42.902 mmol) in anhydrous THE (100 mL) stirring at 0° C. was added Lithium Borohydride (4.9305 g, 215.02 mmol). The reaction mixture was then heated to and stirred at 50° C. for 4 h. The reaction was diluted with DI water (30 mL) and extracted with EtOAc (3×50 mL). The combined EtOAc layers were washed with saturated aqueous NaCl (100 mL), dried over anhydrous sodium sulfate, filtered and concentrated under vacuum. The crude product (8.637 g) was obtained as a light orange solid. (2-bromo-3-methyl-phenyl)methanol (8.637 g, 100%). ¹H NMR (500 MHz, DMSO-d₆) δ 7.37 (d, J=7.5 Hz, 1H), 7.28 (t, J=7.5, 7.5 Hz, 1H), 7.23 (d, J=7.3 Hz, 1H), 5.39 (t, J=5.6, 5.6 Hz, 1H), 4.51 (d, J=5.7 Hz, 2H), 2.35 (s, 3H).

### Step 2: 1-(Benzyloxymethyl)-2-bromo-3-methyl-benzene

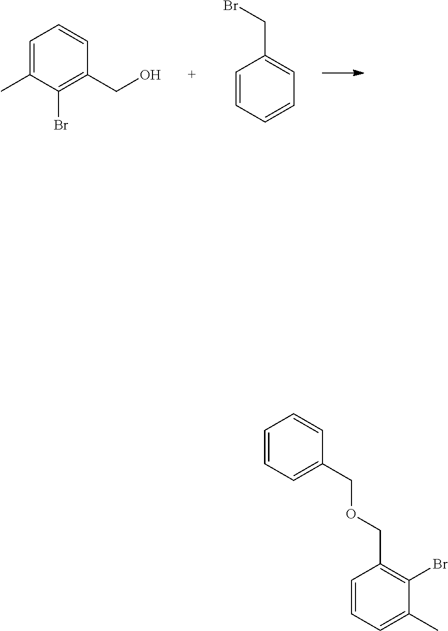

To (2-bromo-3-methyl-phenyl)methanol (1.87 g, 9.301 mmol) in DMSO (38 mL) cooled to 0° C. in an ice bath was added NaH (1.227 g of 60% w/w, 30.68 mmol) and the reaction was stirred for 15 minutes. Then bromomethylbenzene (1.75 mL, 14.71 mmol) was added and the mixture was allowed warm to rt and stir for 16 h. The mixture was partitioned between EtOAc and water. The organic layer was washed with brine, dried (sodium sulfate), filtered and concentrated to a solid which was purified by silica gel chromatography (80 gram column) using a shallow gradient from 100% hexanes to 40% EtOAc (compound elutes at 18% ethyl acetate) giving 1-(benzyloxymethyl)-2-bromo-3-methyl-benzene (2.69 g, 99%). ESI-MS m/z calc. 290.03064, found 291.2 (M+1)⁺; Retention time: 2.06 minutes; LC method A.

### Step 3: 2-[2-(Benzyloxymethyl)-6-methyl-phenyl]-4,4,5,5-tetramethyl-1,3,2-dioxaborolane

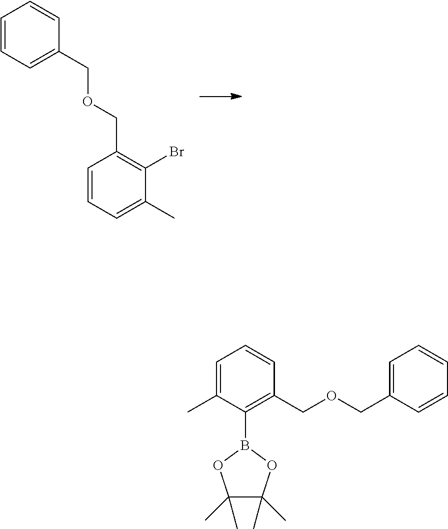 [SMILES: Cc1cccc(COCc2ccccc2)c1Br ; Cc1cccc(COCc2ccccc2)c1B1OC(C)(C)C(C)(C)O1]

In a 350 mL sealed vessel was dissolved 1-(benzyloxymethyl)-2-bromo-3-methyl-benzene (5.4 g, 18.55 mmol) in dioxane (55 mL) and to it was added KOAc (3.85 g, 39.23 mmol) and the mixture was degassed with nitrogen for several minutes. Then bis(pinacol)diboron (7.25 g, 28.55 mmol) was added, followed by Pd(dppf)Cl₂ (1.41 g, 1.932 mmol) and the reaction was purged again by N₂, sealed and heated to 100° C. for 16 hours. After the reaction was cooled to room temperature, saturated ammonium chloride was added, and the reaction was extracted with ethyl acetate. The combined organic extracts were washed with brine, dried over sodium sulfate, filtered and concentrated. The resulting brown oil was purified by silica gel column chromatography (220 gram column) using a gradient of 100% hexanes to 30% ethyl acetate in hexanes (compound elutes at 10% ethyl acetate) to obtain the desired compound as a white solid 2-[2-(benzyloxymethyl)-6-methyl-phenyl]-4,4,5,5-tetramethyl-1,3,2-dioxaborolane (4.63 g, 74%). ESI-MS m/z calc. 338.20532, found 339.4 (M+1)⁺; Retention time: 2.23 minutes; LC method A. ¹H NMR (499 MHz, Chloroform-d) δ 7.37-7.32 (m, 4H), 7.32-7.27 (m, 1H), 7.25-7.20 (m, 1H), 7.10 (dd, J=23.3, 7.5 Hz, 2H), 4.62 (s, 2H), 4.46 (s, 2H), 2.45 (s, 3H), 1.34 (s, 12H).

### Step 4: tert-Butyl N-[4-[2-(benzyloxymethyl)-6-methyl-phenyl]-6-chloro-pyrimidin-2-yl]-N-tert-butoxycarbonyl-carbamate

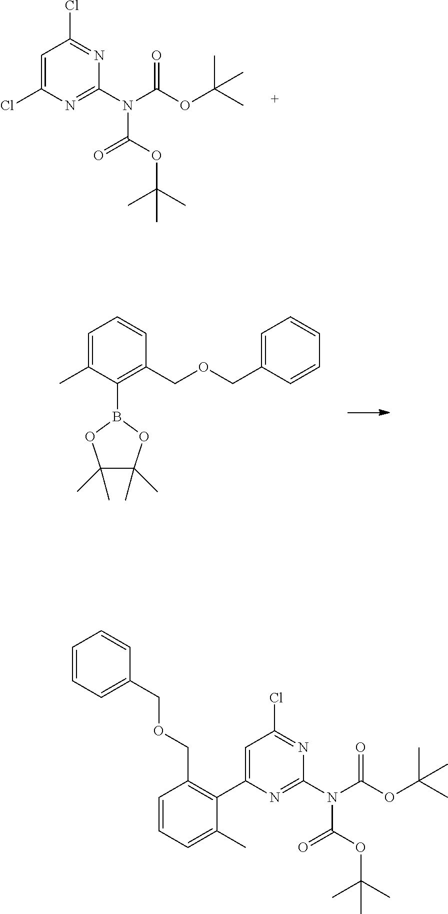 [SMILES: CC(C)(C)OC(=O)N(C(=O)OC(C)(C)C)c1nc(Cl)cc(Cl)n1 ; Cc1cccc(COCc2ccccc2)c1B1OC(C)(C)C(C)(C)O1 ; Cc1cccc(COCc2ccccc2)c1-c1cc(Cl)nc(N(C(=O)OC(C)(C)C)C(=O)OC(C)(C)C)n1]

tert-Butyl N-tert-butoxycarbonyl-N-(4,6-dichloropyrimidin-2-yl)carbamate (1.5 g, 4.118 mmol) and 2-[2-(benzyloxymethyl)-6-methyl-phenyl]-4,4,5,5-tetramethyl-1,3,2-dioxaborolane (1.4 g, 4.139 mmol) were combined in dimethoxyethane (36 mL) and water (6 mL). Added to the mixture were [1,1′-bis(diphenylphosphino)ferrocene]dichloropalladium(II) (315 mg, 0.4305 mmol) and potassium carbonate (1.5 g, 10.85 mmol) and nitrogen was bubbled through the suspension for 1 minute. The reaction was capped and heated to 80° C. for 2 hours. The mixture was cooled to ambient temperature and saturated ammonium chloride was added and the it was extracted with ethyl acetate. The combined organic extracts were washed with brine, dried over sodium sulfate, filtered and concentrated. The residue was purified by silica gel column chromatography (80 gram column) using a gradient of 100% hexanes to 50% ethyl acetate in hexanes (compound elutes at 30% EtOAc) to give a pale-yellow oil tert-butyl N-[4-[2-(benzyloxymethyl)-6-methyl-phenyl]-6-chloro-pyrimidin-2-yl]-N-tert-butoxycarbonyl-carbamate (1.73 g, 78%). ESI-MS m/z calc. 539.2187, found 540.2 (M+1)⁺; Retention time: 1.87 minutes; LC method Q. ¹H NMR (499 MHz, Chloroform-d) δ 7.39-7.35 (m, 2H), 7.35-7.30 (m, 3H), 7.29-7.23 (m, 4H), 4.40 (s, 2H), 4.30 (s, 2H), 2.13 (s, 3H), 1.44 (s, 18H).

### Step 5: 4-[2-(Benzyloxymethyl)-6-methyl-phenyl]-6-chloro-pyrimidin-2-amine

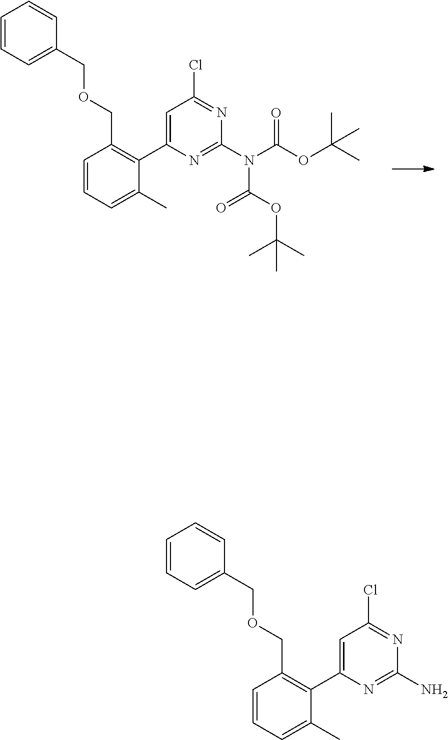 [SMILES: Cc1cccc(COCc2ccccc2)c1-c1cc(Cl)nc(N(C(=O)OC(C)(C)C)C(=O)OC(C)(C)C)n1 ; Cc1cccc(COCc2ccccc2)c1-c1cc(Cl)nc(N)n1]

tert-Butyl N-[4-[2-(benzyloxymethyl)-6-methyl-phenyl]-6-chloro-pyrimidin-2-yl]-N-tert-butoxycarbonyl-carbamate (1.7 g, 3.148 mmol) was dissolved in DCM (35 mL) and to the mixture was added HCl (4M in dioxane) (21 mL of 4 M, 84.00 mmol) and the reaction was stirred at room temperature. After 6 h, the mixture was evaporated to dryness, then diluted with ether (50 mL×2) and then hexanes: dichloromethane (1:1 mixture, 50 mL) and concentrated. The material was then placed on the high vacuum pump for 16 h to afford a pale-yellow gum as product 4-[2-(benzyloxymethyl)-6-methyl-phenyl]-6-chloro-pyrimidin-2-amine (1.07 g, 100%). ESI-MS m/z calc. 339.11383, found 340.2 (M+1)⁺; Retention time: 1.67 minutes; LC method A. ¹H NMR (499 MHz, DMSO-d₆) δ 7.35-7.30 (m, 4H), 7.29-7.24 (m, 2H), 7.24-7.15 (m, 4H), 6.65 (s, 1H), 4.37 (s, 2H), 4.33 (s, 2H), 2.10 (s, 3H).

### Step 6: Methyl 3-[[4-[2-(benzyloxymethyl)-6-methyl-phenyl]-6-chloro-pyrimidin-2-yl]sulfamoyl]benzoate

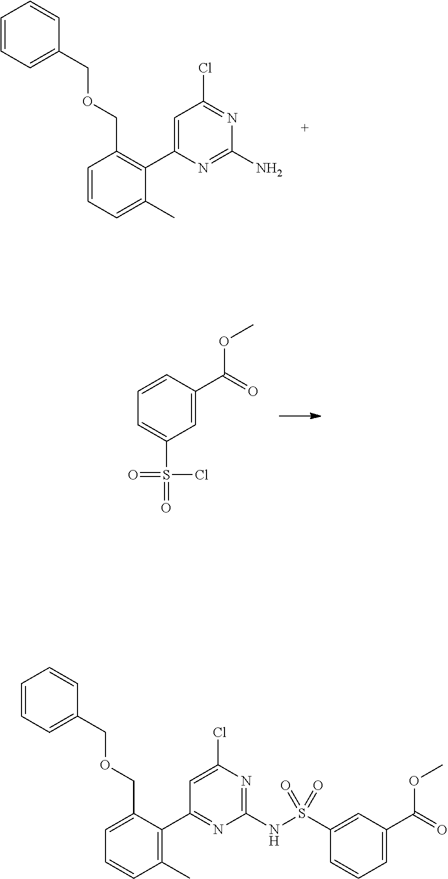 [SMILES: Cc1cccc(COCc2ccccc2)c1-c1cc(Cl)nc(N)n1 ; COC(=O)c1cccc(S(=O)(=O)Cl)c1 ; COC(=O)c1cccc(S(=O)(=O)Nc2nc(Cl)cc(-c3c(C)cccc3COCc3ccccc3)n2)c1]

4-[2-(benzyloxymethyl)-6-methyl-phenyl]-6-chloro-pyrimidin-2-amine (2.74 g, 8.063 mmol) was dissolved in THF (50 mL) and cooled in an ice bath to 0° C. methyl 3-chlorosulfonylbenzoate (2.85 g, 12.15 mmol) was added in one portion. lithium tert-amoxide (7.25 mL of 40% w/w, 22.50 mmol) was added dropwise and the reaction was allowed to slowly warm to room temperature. The reaction was stirred for 6 h. The mixture was pumped on high vacuum overnight, then re-subjected to dilution in THE (25 mL). After cooling in an ice bath to methyl 3-chlorosulfonylbenzoate (1.0 g) was added followed by the addition of lithium tert-amoxide (3 mL of 40% w/w) added dropwise and the reaction was allowed to warm at room temperature for 4 hours. The mixture was acidified the addition of 1 HCl. The reaction mixture was extracted with ethyl acetate. The organics were washed with brine, dried over sodium sulfate and evaporated. The crude material was purified utilizing silica gel column chromatography (120 gram column) using a gradient of 100% hexanes to 80% ethyl acetate in hexanes (compound elutes at 50% EtOAc) to give a pale-yellow oil which solidified upon high vacuum to produce methyl 3-[[4-[2-(benzyloxymethyl)-6-methyl-phenyl]-6-chloro-pyrimidin-2-yl]sulfamoyl]benzoate (2.06 g, 47%). ESI-MS m/z calc. 537.11255, found 538.2 (M+1)⁺; Retention time: 1.97 minutes; LC method A.

### Step 7: 3-[[4-[2-(Benzyloxymethyl)-6-methyl-phenyl]-6-chloro-pyrimidin-2-yl]sulfamoyl]benzoic acid

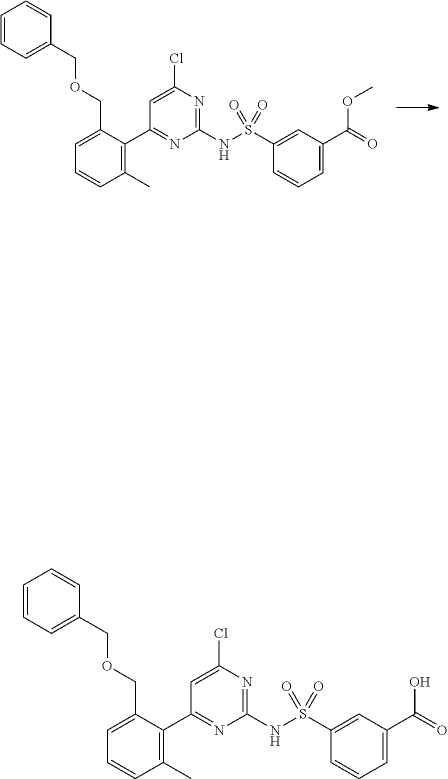 [SMILES: COC(=O)c1cccc(S(=O)(=O)Nc2nc(Cl)cc(-c3c(C)cccc3COCc3ccccc3)n2)c1 ; Cc1cccc(COCc2ccccc2)c1-c1cc(Cl)nc(NS(=O)(=O)c2cccc(C(=O)O)c2)n1]

Methyl 3-[[4-[2-(benzyloxymethyl)-6-methyl-phenyl]-6-chloro-pyrimidin-2-yl]sulfamoyl]benzoate (2.06 g, 3.829 mmol) and NaOH (30 mL of 1 M, 30.00 mmol) were combined in THE (25 mL) and stirred at room temperature for 2 h. The reaction was made acidic by the addition of 1M HCl and extracted with ethyl acetate. The organics were washed with brine, dried over sodium sulfate and evaporated. The material was placed on high vac overnight to give 3-[[4-[2-(benzyloxymethyl)-6-methyl-phenyl]-6-chloro-pyrimidin-2-yl]sulfamoyl]benzoic acid (1.95 g, 97%). ESI-MS m/z calc. 523.09686, found 524.1 (M+1)⁺; Retention time: 1.72 minutes; LC method A.

### Step 8: 3-[[4-[(2R)-2-Amino-4-methyl-pentoxy]-6-[2-(benzyloxymethyl)-6-methyl-phenyl]pyrimidin-2-yl]sulfamoyl]benzoic acid

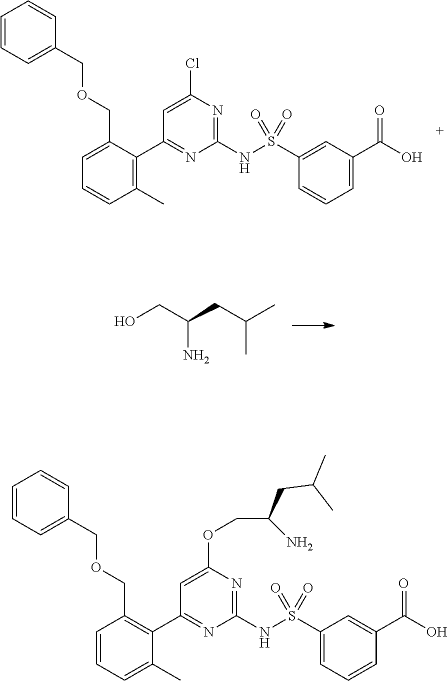

In a 500 mL flask, 3-[[4-[2-(benzyloxymethyl)-6-methyl-phenyl]-6-chloro-pyrimidin-2-yl]sulfamoyl]benzoic acid (2.0 g, 3.817 mmol), (2R)-2-amino-4-methyl-pentan-1-ol (460 mg, 3.925 mmol) and THE (40 mL) were mixed and cooled in an ice bath at 0° C., to which KOtBu (2.15 g, 19.16 mmol) was added. This mixture was stirred 2 h. The reaction was acidified by the addition of HCl (4M in dioxane) (7 mL of 4 M, 28.00 mmol), stirred for 15 minutes and then concentrated in vacuo. The material was dissolved in DCM/ether and triturated, filtered and dried under high vacuum to afford off-white solid 3-[[4-[(2R)-2-amino-4-methyl-pentoxy]-6-[2-(benzyloxymethyl)-6-methyl-phenyl]pyrimidin-2-yl]sulfamoyl]benzoic acid (hydrochloride salt) (2.4 g, 98%). ESI-MS m/z calc. 604.23553, found 605.2 (M+1)⁺; Retention time: 1.29 minutes; LC method A.

### Example O: Preparation of tert-butyl 2-[[(1R)-4,4,4-trifluoro-1-(hydroxymethyl)-3,3-dimethyl-butyl]amino]-7-azaspiro[3.5]nonane-7-carboxylate

### Step 1: tert-Butyl 2-[[(1R)-4,4,4-trifluoro-1-(hydroxymethyl)-3,3-dimethyl-butyl]amino]-7-azaspiro[3.5]nonane-7-carboxylate

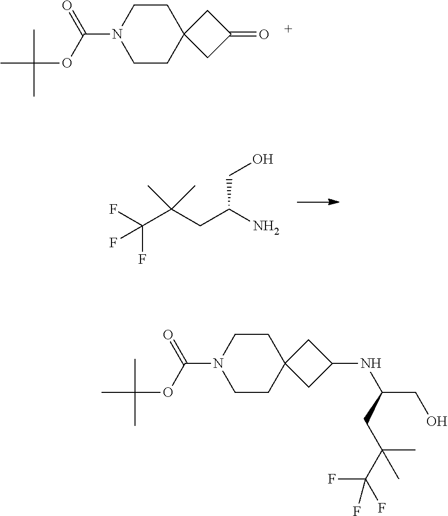 [SMILES: CC(C)(C)OC(=O)N1CCC2(CC1)CC(=O)C2 ; CC(C)(C[C@@H](N)CO)C(F)(F)F ; CC(C)(C)OC(=O)N1CCC2(CC1)CC(N[C@@H](CO)CC(C)(C)C(F)(F)F)C2]

In a 250 mL flask, (2R)-2-amino-5,5,5-trifluoro-4,4-dimethyl-pentan-1-ol (hydrochloride salt) (1.513 g, 6.826 mmol) was added to a solution of tert-butyl 2-oxo-7-azaspiro[3.5]nonane-7-carboxylate (1.496 g, 6.251 mmol) in anhydrous DCE (10 mL) under nitrogen and stirred at rt for 20 minutes (cloudy solution). Sodium triacetoxyborohydride (3.98 g, 18.78 mmol) was divided into 3 separate portions and added to give a thick suspension (magnetic stirring still efficient). The mixture was stirred at rt for 22 hours. The mixture was cooled in an ice bath (internal temp=2° C.), then HCl (10 mL of 4 M, 40.00 mmol) was added very slowly, keeping the temperature between 2° C. and 6° C. A solution of potassium carbonate (10 g, 72.36 mmol) in water (10 mL) was added, while keeping the temperature below 10° C., then another portion of water (15 mL) (final pH=11) was added, followed by DCM (20 mL). The two phases were separated. The aqueous phase was further extracted with DCM (30 mL). The combined extracts were washed with saturated sodium bicarbonate (30 mL), dried over sodium sulfate and the solvents were evaporated. Drying under vacuum gave crude tert-butyl 2-[[(1R)-4,4,4-trifluoro-1-(hydroxymethyl)-3,3-dimethyl-butyl]amino]-7-azaspiro[3.5]nonane-7-carboxylate (2.446 g, 96%) as a brown honey-like resin. ESI-MS m/z calc. 408.25998, found 409.26 (M+1)⁺; Retention time: 1.32 minutes; LC method A. ¹H NMR (400 MHz, DMSO-d₆) δ 4.54 (t, J=5.5 Hz, 1H), 3.90 (s, 2H), 3.31-3.13 (m, 7H), 2.10-2.02 (m, 1H), 1.63-1.51 (m, 2H), 1.47-1.27 (m, 15H), 1.19-1.12 (m, 3H), 1.12-1.00 (m, 3H).

### Example P: Preparation of 6-[[4-[(2R)-2-amino-4,4-dimethyl-pentoxy]-6-(2,6-dimethylphenyl)pyrimidin-2-yl]sulfamoyl]pyridine-2-carboxylic acid

### Step 1: 6-[[4-[(2R)-2-Amino-4,4-dimethyl-pentoxy]-6-(2,6-dimethylphenyl)pyrimidin-2-yl]sulfamoyl]pyridine-2-carboxylic acid

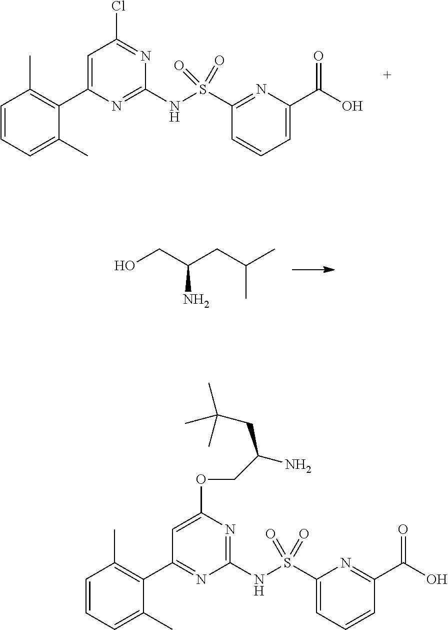

6-[[4-chloro-6-(2,6-dimethylphenyl)pyrimidin-2-yl]sulfamoyl]pyridine-2-carboxylic acid (5.03 g, 12.01 mmol) and (2R)-2-amino-4,4-dimethyl-pentan-1-ol (hydrochloride salt) (2.05 g, 12.23 mmol) were combined in THE (35 mL). To the resulting suspension (hard to stir), sodium tert-butoxide (4.62 g, 48.07 mmol) was added in 3 equal portions resulting in partial dissolution of the solids and a slightly exothermic reaction. The mixture was stirred at room temperature for 5 hours (cloudy suspension). More (2R)-2-amino-4,4-dimethyl-pentan-1-ol (hydrochloride salt) (338 mg, 2.016 mmol) and sodium tert-butoxide (Sodium salt) (610 mg, 6.347 mmol) were added and the mixture was stirred for an additional 1.5 h. The reaction was diluted with ethyl acetate (80 mL), HCl (75 mL of 1 M, 75.00 mmol) and brine (50 mL) and the resulting two phases separated. The aqueous phase was further extracted with EtOAc (3×20 mL). The combined organic extracts were dried over sodium sulfate and concentrated. The residue was triturated in a 1:3 EtOAc: hexanes mixture and stirred in this solvent mixture over the weekend. The solid was filtered and dried to give 6-[[4-[(2R)-2-amino-4,4-dimethyl-pentoxy]-6-(2,6-dimethylphenyl)pyrimidin-2-yl]sulfamoyl]pyridine-2-carboxylic acid (hydrochloride salt) (6.397 g, 97%) as a white solid. ESI-MS m/z calc. 513.2046, found 514.6 (M+1)⁺; Retention time: 1.05 minutes; LC method A. ¹H NMR (400 MHz, DMSO-d₆) δ 13.36 (broad s, 1H), 8.43-7.87 (m, 6H), 7.28 (t, J=7.6 Hz, 1H), 7.14 (d, J=7.6 Hz, 2H), 6.31 (s, 1H), 4.20 (dd, J=12.3, 2.9 Hz, 1H), 4.09-3.91 (m, 1H), 3.61 (s, 1H), 2.03 (s, 6H), 1.57 (dd, J=14.7, 7.3 Hz, 1H), 1.46 (dd, J=14.6, 3.7 Hz, 1H), 0.93 (s, 9H).

### V. Synthesis of New Compounds

### Example 1: Preparation of Compound 1

### Step 1: tert-Butyl N-[(1R)-1-(cyclohexylmethyl)-2-[methoxy(methyl)amino]-2-oxo-ethyl]carbamate

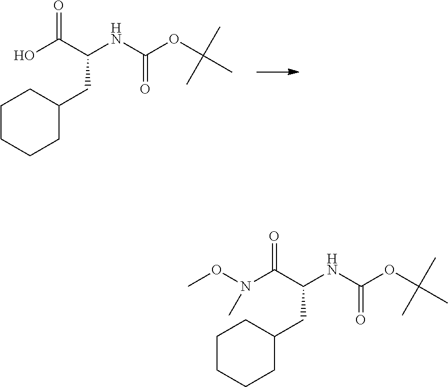 [SMILES: CC(C)(C)OC(=O)N[C@H](CC1CCCCC1)C(=O)O ; CON(C)C(=O)[C@@H](CC1CCCCC1)NC(=O)OC(C)(C)C]

To a solution of (2R)-2-(tert-butoxycarbonylamino)-3-cyclohexyl-propanoic acid (10.36 g, 38.18 mmol) and 1-hydroxybenzotriazole (5.5 g, 40.70 mmol) in DMF (120 mL) was added DIPEA (20 mL, 114.8 mmol) followed by EDCI-HCl (7.9 g, 41.21 mmol) then N-methoxymethanamine (hydrochloride salt) (4.9 g, 50.23 mmol) and DIPEA (10 mL, 57.41 mmol), and the reaction mixture was stirred at room temperature for 16 h. The reaction mixture was poured in to 0.1 N HCl (500 mL), the pH adjusted to 4 with 1 N HCl and then extracted with EtOAc (3×). The organics were combined, washed with 0.1 N HCl, water, saturated aqueous sodium bicarbonate (2×), brine, dried over sodium sulfate and evaporated to dryness. Purification by column chromatography (220 g silica; 0-30% EtOAc in hexanes) gave a clear oil, tert-butyl N-[(1R)-1-(cyclohexylmethyl)-2-[methoxy(methyl)amino]-2-oxo-ethyl]carbamate (11.1 g, 93%); ¹H NMR (400 MHz, Chloroform-d) δ 5.03 (d, J=9.6 Hz, 1H), 4.75 (s, 1H), 3.78 (s, 3H), 3.20 (s, 3H), 1.91 (d, J=12.9 Hz, 1H), 1.76-1.59 (m, 4H), 1.57-1.32 (m, 12H), 1.32-1.08 (m, 3H), 1.02-0.82 (m, 2H). ESI-MS m/z calc. 314.22055, found 315.3 (M+1)⁺; Retention time: 0.69 minutes; LC method D.

### Step 2: (1R,2R)-2-Amino-3-cyclohexyl-1-cyclopropyl-propan-1-ol

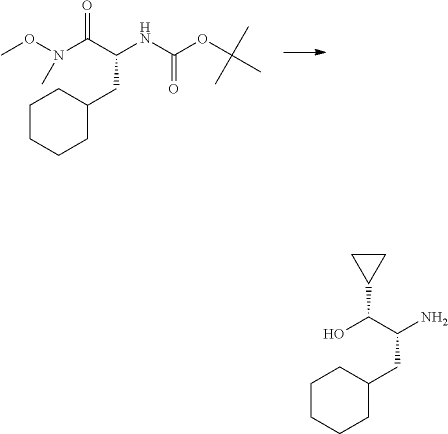 [SMILES: CON(C)C(=O)[C@@H](CC1CCCCC1)NC(=O)OC(C)(C)C ; N[C@H](CC1CCCCC1)[C@H](O)C1CC1]

Stage 1: A THE (12 mL) solution of tert-butyl N-[(1R)-1-(cyclohexylmethyl)-2-[methoxy(methyl)amino]-2-oxo-ethyl]carbamate (2.18 g, 6.933 mmol) was cooled to 0° C. using an ice-water bath and treated with a solution of LAH in THE (7 mL of 1 M, 7.00 mmol) dropwise. The reaction was stirred for 30 min and then quenched with citric acid (15 mL of 1 M, 15.00 mmol) carefully. The mixture was extracted with ethyl acetate (3×50 mL). The combined organic extracts were washed with water (100 mL) and saturated aqueous sodium chloride solution (100 mL), then dried over sodium sulfate, filtered, and evaporated in vacuo to give a light yellow oil. The aldehyde product was used in the next step without further purification.

Stage 2: The Stage 1 product from above was taken up in THE (12 mL), cooled to 0° C. and treated with bromo(cyclopropyl)magnesium in MeTHF (15 mL of 1 M, 15.00 mmol) and the reaction was warmed to room temperature and stirred for 2 h. Then, it was quenched with aqueous HCl (20 mL of 1 M, 20.00 mmol) and diluted with ethyl acetate (15 mL). The organic phase was separated and washed with water (10 mL) followed by brine (10 mL). The organic layer was dried over anhydrous sodium sulfate, filtered, and concentrated in vacuo. The crude product was used in the next step without further purification.

Stage 3: The Stage 2 product from above was treated with HCl in dioxane (approximately 1.733 mL of 4 M, 6.933 mmol), stirred at room temperature for 90 min and then concentrated in vacuo. The residue was taken up in MeOH (3 mL) purified by reverse-phase preparative HPLC (C₁₈) to afford (1R,2R)-2-amino-3-cyclohexyl-1-cyclopropyl-propan-1-ol (hydrochloride salt) (170.8 mg, 11%); ¹H NMR (400 MHz, DMSO-d₆) δ 7.76 (s, 2H), 3.15-2.97 (m, 1H), 2.87 (dd, J=7.8, 5.4 Hz, 1H), 1.71-1.57 (m, 5H), 1.56-1.47 (m, 1H), 1.46-1.33 (m, 2H), 1.27-1.05 (m, 4H), 0.96-0.73 (m, 3H), 0.52-0.38 (m, 2H), 0.34-0.28 (m, 1H), 0.28-0.16 (m, 1H). ESI-MS m/z calc. 197.17796, found 198.2 (M+1)⁺; Retention time: 1.01 minutes; LC method A.

### Step 3: (1R,2R)-3-Cyclohexyl-1-cyclopropyl-2-(spiro[2.3]hexan-5-ylamino)propan-1-Ol

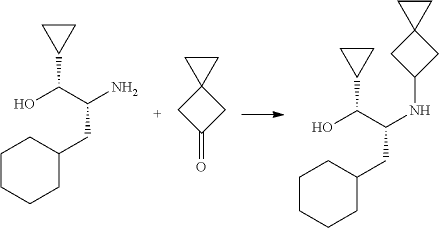 [SMILES: N[C@H](CC1CCCCC1)[C@H](O)C1CC1 ; O=C1CC2(CC2)C1 ; O[C@H](C1CC1)[C@@H](CC1CCCCC1)NC1CC2(CC2)C1]

To a solution of spiro[2.3]hexan-5-one (13.2 mg, 0.1373 mmol) in DCM (0.5 mL) was added (1R,2R)-2-amino-3-cyclohexyl-1-cyclopropyl-propan-1-ol (hydrochloride salt) (33.7 mg, 0.1442 mmol), followed by sodium triacetoxyborohydride (51.2 mg, 0.2416 mmol) and the mixture was stirred for 3 h. The volatiles were removed in vacuo, then the residue was filtered and purified using reverse-phase preparative HPLC (Cis; HCl modifier was critical to prevent isomerization to give (1R,2R)-3-cyclohexyl-1-cyclopropyl-2-(spiro[2.3]hexan-5-ylamino)propan-1-ol (30.2 mg, 75%) ESI-MS m/z calc. 277.24057, found 278.3 (M+1)⁺; Retention time: 0.55 minutes; LC method D.

### Step 4: 3-[[4-[(1R,2R)-3-Cyclohexyl-1-cyclopropyl-2-(spiro[2.3]hexan-5-ylamino)propoxy]-6-(2,6-dimethylphenyl)pyrimidin-2-yl]sulfamoyl]benzoic acid

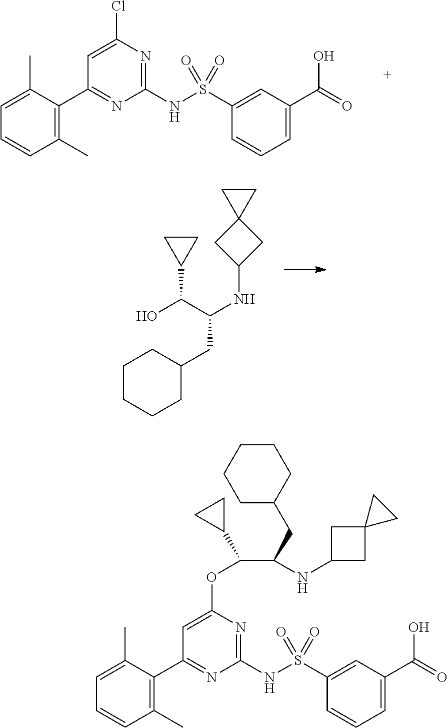

A THE (2 mL) mixture of (1R,2R)-3-cyclohexyl-1-cyclopropyl-2-(spiro[2.3]hexan-5-ylamino)propan-1-ol (36.2 mg, 0.1305 mmol), 3-[[4-chloro-6-(2,6-dimethylphenyl)pyrimidin-2-yl]sulfamoyl]benzoic acid (66.5 mg, 0.1591 mmol), and sodium t-butoxide (62.6 mg, 0.6514 mmol) was stirred at room temperature for 2 h. The solutions were filtered and the filtrate was diluted with 0.8 mL MeOH, and purified by reverse-phase preparative HPLC (C₁₈) to give 3-[[4-[(1R,2R)-3-cyclohexyl-1-cyclopropyl-2-(spiro[2.3]hexan-5-ylamino)propoxy]-6-(2,6-dimethylphenyl)pyrimidin-2-yl]sulfamoyl]benzoic acid (hydrochloride salt) (23.9 mg, 26%) ESI-MS m/z calc. 658.3189, found 659.6 (M+1)⁺; Retention time: 2.05 minutes; LC method A.

### Step 5: (10R,11R)-11-(Cyclohexylmethyl)-10-cyclopropyl-6-(2,6-dimethylphenyl)-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (Compound 1)

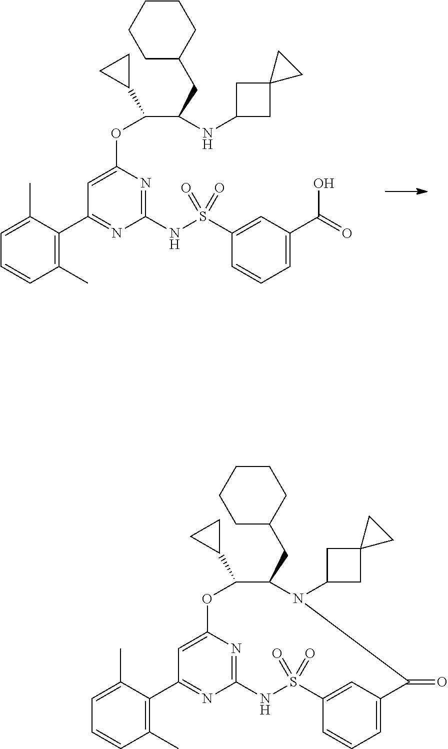 [SMILES: Cc1cccc(C)c1-c1cc(O[C@H](C2CC2)[C@@H](CC2CCCCC2)NC2CC3(CC3)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CC3)C1)[C@H](CC1CCCCC1)[C@@H](C1CC1)O2]

A DMF (1.8 mL) solution of 3-[[4-[(1R,2R)-3-cyclohexyl-1-cyclopropyl-2-(spiro[2.3]hexan-5-ylamino)propoxy]-6-(2,6-dimethylphenyl)pyrimidin-2-yl]sulfamoyl]benzoic acid (hydrochloride salt) (13.5 mg, 0.01942 mmol), COMU (13.2 mg, 0.03082 mmol), and triethylamine (20 μL, 0.1435 mmol) was stirred at room temperature for 1 h. The solutions were filtered and the filtrate was purified by reverse-phase preparative HPLC (C₁₈) to give (10R,11R)-11-(cyclohexylmethyl)-10-cyclopropyl-6-(2,6-dimethylphenyl)-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (3.8 mg, 31%) ESI-MS m/z calc. 640.30835, found 641.7 (M+1)⁺; Retention time: 2.39 minutes; LC method A.

### Example 2: Preparation of Compound 2

### Step 1: 3-Methyl-2-(4,4,5,5-tetramethyl-1,3,2-dioxaborolan-2-yl)benzaldehyde

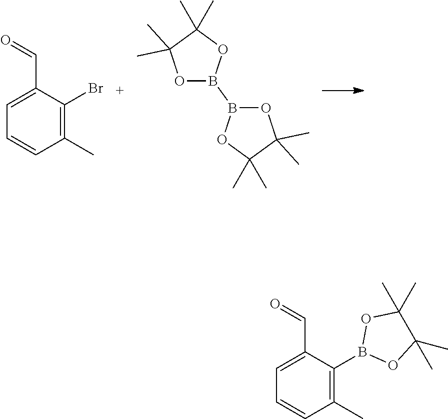

A solution of 2-bromo-3-methyl-benzaldehyde (22.5 g, 113.04 mmol), bis(pinacolato)diboron (43.1 g, 169.73 mmol), and KOAc (22.2 g, 226.20 mmol) in 1,4-dioxane (500 mL) was prepared. The resulting slurry was sparged with a nitrogen stream for five minutes, then Pd(dppf)Cl₂ (8.3 g, 11.343 mmol) was added and the mixture was refluxed under nitrogen for twenty hours, then cooled to room temperature and quenched with 1M hydrochloric acid until the pH was approximately 3-4. The phases were then separated: the aqueous phase was discarded and the organic phase was concentrated in vacuo, combined with the crude product from another reaction run on 2.5 g, and purified by silica gel chromatography using 0 to 10% ethyl acetate in hexane to obtain 3-methyl-2-(4,4,5,5-tetramethyl-1,3,2-dioxaborolan-2-yl)benzaldehyde (22.5 g, 81%) as a pale-yellow oil. ¹H NMR (500 MHz, Chloroform-d) δ 9.98 (s, 1H), 7.63 (dd, J=6.6, 2.1 Hz, 1H), 7.43 (d, J=6.6 Hz, 2H), 2.49 (s, 3H), 1.49 (s, 12H). The product still contains ˜25 mole % of bis(pinacolato)diboron. ESI-MS m/z calc. 246.14273, found 247.2 (M+1)⁺; Retention time: 0.66 minutes; LC method S.

### Step 2: 2-[(11R)-11-Isobutyl-3-(methoxymethyl)-2,2,13-trioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-6-yl]-3-methyl-benzaldehyde

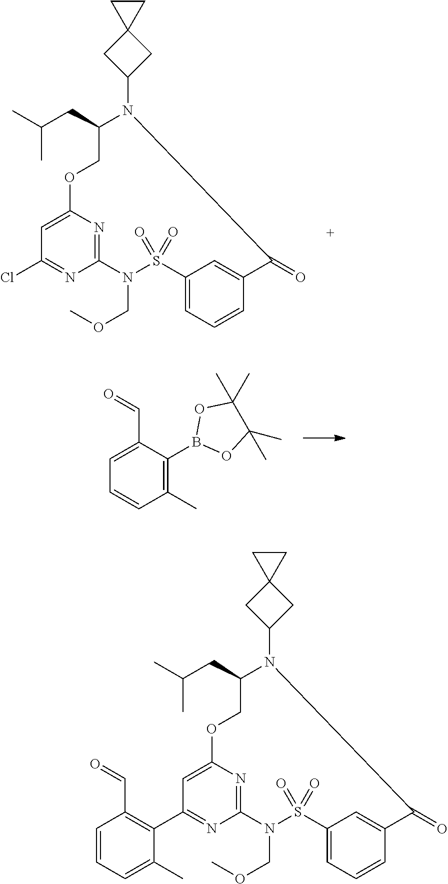

A heterogeneous solution of (11R)-6-chloro-11-isobutyl-3-(methoxymethyl)-2,2-dioxo-12-spiro[2.3]hexan-5-yl-9-oxa-2λ⁶-thia-3,5,12,19-tetrazatricyclo[12.3.1.14,8]nonadeca-1(18),4,6,8(19),14,16-hexaen-13-one (90 mg, 0.1682 mmol), 3-methyl-2-(4,4,5,5-tetramethyl-1,3,2-dioxaborolan-2-yl)benzaldehyde (78 mg, 0.3169 mmol), potassium carbonate (77 mg, 0.5571 mmol), and Pd(dppf)Cl₂ (26 mg, 0.0318 mmol) in DMA (2 mL) was heated to 100° C. in a heating block for 0.5 hours, quenched with a small amount of 1M hydrochloric acid, then diluted with DCM (10 mL). The phases were separated: and the organic phase was concentrated in vacuo. The crude material was combined with other batches from reactions run on a similar scale, before silica gel chromatography (0-60% diethyl ether in hexane) to obtain 2-[(11R)-11-isobutyl-3-(methoxymethyl)-2,2,13-trioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-6-yl]-3-methyl-benzaldehyde (65 mg, 62%) ESI-MS m/z calc. 618.2512, found 619.7 (M+1)⁺; Retention time: 7.46 minutes; LC method S.

### Step 3: (11R)-6-[2-(Hydroxymethyl)-6-methyl-phenyl]-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (Compound 2)

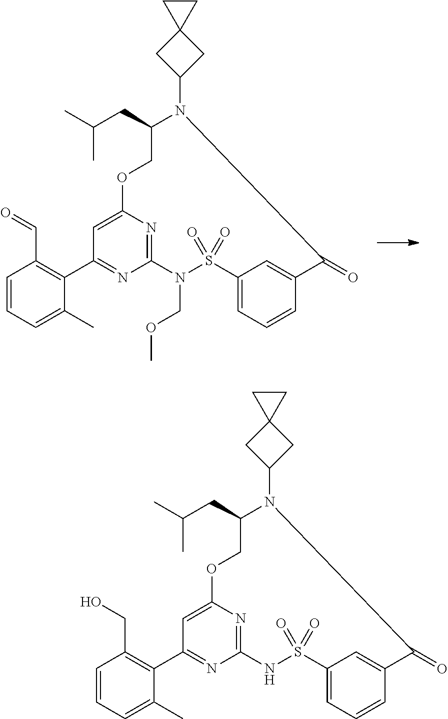 [SMILES: COCN1c2nc(cc(-c3c(C)cccc3C=O)n2)OC[C@@H](CC(C)C)N(C2CC3(CC3)C2)C(=O)c2cccc(c2)S1(=O)=O ; Cc1cccc(CO)c1-c1cc2nc(n1)NS(=O)(=O)c1cccc(c1)C(=O)N(C1CC3(CC3)C1)[C@H](CC(C)C)CO2]

A solution of 2-[(11R)-11-isobutyl-3-(methoxymethyl)-2,2,13-trioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-6-yl]-3-methyl-benzaldehyde (93 mg, 0.1618 mmol) and sodium borohydride (31 mg, 0.0328 mL, 0.8194 mmol) in ethanol (10 mL) was stirred at room temperature for 1.5 hours. The reaction mixture was quenched with a small amount of hydrochloric acid and then concentrated in vacuo to obtain (11R)-6-[2-(hydroxymethyl)-6-methyl-phenyl]-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (93 mg, 70%) as a yellow solid (mixture of MOM protected and unprotected material). This material was treated with HCl (1 mL of 12 M, 12.000 mmol) in ethanol (5 mL) was stirred at room temperature for 2.5 hours, then was concentrated in vacuo and was purified by HPLC (5-100% acetonitrile in water w/0.1% HCl buffer) to obtain (11R)-6-[2-(hydroxymethyl)-6-methyl-phenyl]-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (28 mg, 30%). H NMR (500 MHz, Chloroform-d) δ 8.68 (s, 1H), 8.00 (d, J=7.9 Hz, 1H), 7.90 (d, J=7.6 Hz, 1H),7.64 (t, J=7.7 Hz, 1H), 7.48 (d, J=7.6 Hz, 1H), 7.39 (t, J=7.6 Hz, 1H), 7.26 (d, J=7.6 Hz, 1H), 6.36 (s, 1H), 5.38 (dd, J=10.8, 4.1 Hz, 1H), 4.48 (d, J=12.0 Hz, 1H), 4.31 (d, J=12.1 Hz, 1H), 4.20-4.06 (m, 2H), 3.86 (td, J=9.6, 7.8, 5.7 Hz, 1H), 3.27 (dt, J=25.9, 9.5 Hz, 2H), 2.26-2.21 (m, 1H), 2.19 (s, 3H), 2.18-2.12 (m, 1H), 1.70 (ddd, J=14.2, 11.0, 2.8 Hz, 1H), 1.46 (tt, J=6.5, 3.1 Hz, 1H), 1.28 (ddd, J=13.9, 10.6, 2.9 Hz, 1H), 0.83 (d, J=6.6 Hz, 3H), 0.56 (d, J=3.4 Hz, 4H), 0.35 (d, J=6.4 Hz, 3H). ESI-MS m/z calc. 576.24066, found 577.6 (M+1)⁺; Retention time: 2.56 minutes; LC method W.

### Example 3: Preparation of Compound 3

### Step 1: (2R)-2-Amino-5-methyl-hexan-1-ol (hydrochloride salt)

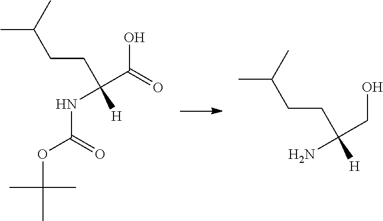

Stage 1: (2R)-2-(tert-Butoxycarbonylamino)-5-methyl-hexanoic acid (1.8 g, 7.337 mmol) was dissolved in THF (15 mL), cooled in an ice bath, and BH₃ (22.5 mL of 1 M, 22.50 mmol) was added dropwise. After the addition was complete, the ice bath was removed, and the reaction mixture was stirred at room temperature for 2 hours. The reaction was then cooled again to 0° C., and methanol was added dropwise (bubbled vigorously). The reaction was allowed to slowly warm to room temperature over an hour, then was concentrated under reduced pressure. 10 mL of methanol was added, and the reaction mixture was again concentrated (twice). This process was repeated three times with 10 mL THE and concentrating under reduced pressure to give a colorless oil, tert-butyl N-[(1R)-1-(hydroxymethyl)-4-methyl-pentyl]carbamate (1.691 g, 100%) ESI-MS m/z calc. 231.18344, found 232.3 (M+1)⁺; Retention time: 0.57 minutes (LC method D).

Stage 2: The product was dissolved in dichloromethane (15 mL) and HCl (22 mL of 4 M, 88.00 mmol) in dioxane was added. The reaction mixture was stirred at room temperature for 30 minutes, then concentrated. Hexanes and dichloromethane were added, and the reaction mixture was concentrated a second time to give the boc-protected material with some residual solvent, used in next step without further purification. (2R)-2-amino-5-methyl-hexan-1-ol (hydrochloride salt) (1.21 g, 98%). ¹H NMR (400 MHz, DMSO) δ 7.89 (s, 2H), 3.58 (dd, J=11.5, 3.8 Hz, 1H), 3.47 (ddt, J=28.1, 11.5, 5.7 Hz, 1H), 3.40-3.30 (m, 1H), 3.00 (dq, J=11.2, 6.1 Hz, 1H), 1.50 (qd, J=6.7, 3.9 Hz, 3H), 1.28-1.15 (m, 2H), 0.87 (dt, J=6.5, 1.7 Hz, 6H).

### Step 2: 3-[[4-[(2R)-2-Amino-5-methyl-hexoxy]-6-(2,6-dimethylphenyl)pyrimidin-2-yl]sulfamoyl]benzoic acid

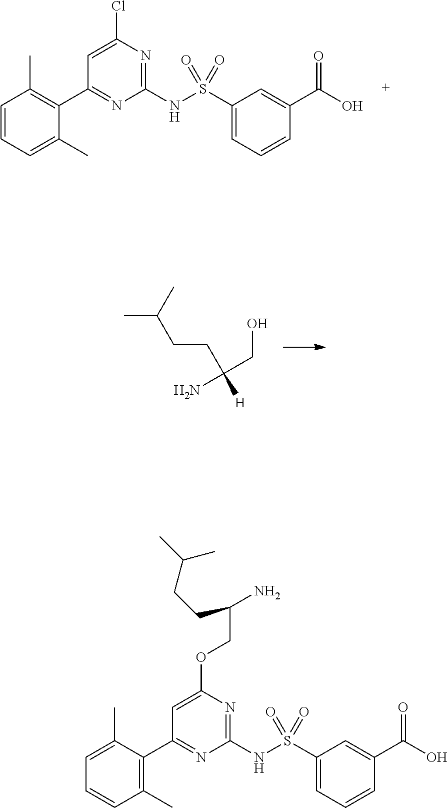

Stage 1: 3-[[4-chloro-6-(2,6-dimethylphenyl)pyrimidin-2-yl]sulfamoyl]benzoic acid (1.495 g, 3.578 mmol), (2R)-2-amino-5-methyl-hexan-1-ol (hydrochloride salt) (1.2 g, 7.157 mmol), and Sodium tert-butoxide (1.725 g, 17.95 mmol) were combined in anhydrous THE (8.418 mL), and warmed to 60° C. for 15 (list 1) to 30 (list 2) minutes. The reaction mixture was then cooled to room temperature, and Boc anhydride (1.565 g, 7.171 mmol) was added. After stirring at room temperature for 2 hours, additional Boc anhydride (700 mg, 3.207 mmol) and sodium tert-butoxide (600 mg, 6.243 mmol) were added and stirring was continued for an additional 2 hours. The reaction mixture was poured into a separatory funnel containing 0.2 M HCl and ethyl acetate. The layers were separated, and the aqueous was extracted an additional 4× ethyl acetate, and the combined organics were washed with brine, dried over sodium sulfate and concentrated. The resulting crude material was purified by column chromatography on silica gel, eluting with 0-100% ethyl acetate in hexanes. Fractions containing product were combined and concentrated.

Stage 2: The resulting crude material was dissolved in dichloromethane (10 mL), HCl (18 mL of 4 M, 72.00 mmol) (in dioxane) was added and the reaction was stirred at room temperature for a 30 minutes, then concentrated under reduced pressure, suspended in dichloromethane and hexanes and concentrated a second time, before drying overnight under high vacuum to give as a white solid, 3-[[4-[(2R)-2-amino-5-methyl-hexoxy]-6-(2,6-dimethylphenyl)pyrimidin-2-yl]sulfamoyl]benzoic acid (hydrochloride salt) (750 mg, 38%). ESI-MS m/z calc. 512.20935, found 513.4 (M+1)⁺; Retention time: 0.44 minutes; LC method D. ¹H NMR (400 MHz, DMSO) δ 8.46 (q, J=1.8 Hz, 1H), 8.21 (s, 2H), 8.18-8.10 (m, 2H), 7.70 (t, J=7.8 Hz, 1H), 7.26 (t, J=7.7 Hz, 1H), 7.13 (d, J=7.6 Hz, 2H), 6.33 (d, J=17.4 Hz, 1H), 4.41-4.33 (m, 1H), 4.23 (dd, J=11.9, 6.5 Hz, 1H), 3.74-3.70 (m, 1H), 3.52-3.44 (m, 3H), 2.01 (s, 6H), 1.60 (ddt, J=10.4, 7.8, 3.8 Hz, 2H), 1.55-1.48 (m, 1H), 1.31-1.17 (m, 2H), 0.87 (dt, J=6.8, 2.4 Hz, 6H).

### Step 3: (11R)-6-(2,6-Dimethylphenyl)-11-isopentyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (Compound 3)

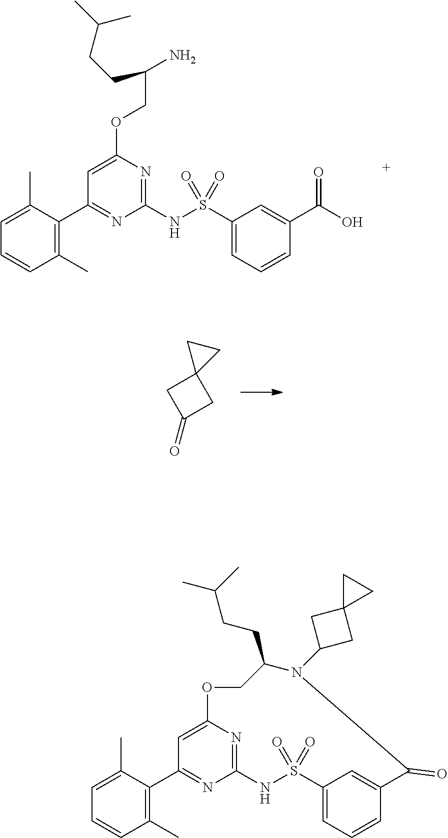 [SMILES: Cc1cccc(C)c1-c1cc(OC[C@H](N)CCC(C)C)nc(NS(=O)(=O)c2cccc(C(=O)O)c2)n1 ; O=C1CC2(CC2)C1 ; Cc1cccc(C)c1-c1cc2nc(n1)NS(=O)(=O)c1cccc(c1)C(=O)N(C1CC3(CC3)C1)[C@H](CCC(C)C)CO2]

Stage 1: 3-[[4-[(2R)-2-amino-5-methyl-hexoxy]-6-(2,6-dimethylphenyl)pyrimidin-2-yl]sulfamoyl]benzoic acid (hydrochloride salt) (60 mg, 0.1093 mmol) and spiro[2.3]hexan-5-one (approximately 21.01 mg, 0.2186 mmol) were combined in dichloromethane (0.5 mL), and sodium triacetoxyborohydride (approximately 46.33 mg, 0.2186 mmol) was added. The reaction mixture was stirred at room temperature for 1 hour, then a second portion of sodium triacetoxyborohydride (approximately 46.33 mg, 0.2186 mmol) was added, and the reaction mixture was stirred at room temperature for an additional hour. An additional portion of spiro[2.3]hexan-5-one (approximately 21.01 mg, 0.2186 mmol) was added, followed 30 minutes later by a third portion of sodium triacetoxyborohydride (approximately 46.33 mg, 0.2186 mmol). After a total of four hours reaction time, the reaction mixture was added to a separatory funnel containing ethyl acetate and 0.5 M HCl. The layers were separated, and the aqueous was extracted an additional 3× with ethyl acetate. The combined organics were washed with brine, dried over sodium sulfate, and concentrated. The resulting solid was used in the next step without further purification.

Stage 2: The crude product was combined with HATU (approximately 66.50 mg, 0.1749 mmol) in DMF and DIPEA (approximately 84.76 mg, 114.2 μL, 0.6558 mmol) was added. The reaction was stirred at room temperature for 3 hours. The reaction mixture was then added to a separatory funnel containing 25 mL 0.5 M HCl and 25 mL ethyl acetate. The layers were separated and the aqueous was extracted 2×15 mL ethyl acetate, and the combined organics were washed with water, brine, and dried over sodium sulfate then concentrated. The resulting crude was purified by reverse phase HPLC (1-99% ACN in water, HCl modifier, 15 min run) to give (11R)-6-(2,6-dimethylphenyl)-11-isopentyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (5 mg, 8%). ESI-MS m/z calc. 574.26135, found 575.4 (M+1)⁺; Retention time: 2.11 minutes; LC method A.

### Example 4: Preparation of Compound 4 and Compound 5

### Step 1: Methyl 6-[(11R)-6-(2,6-dimethylphenyl)-11-(3-methylbutyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]spiro[3.3]heptane-2-carboxylate

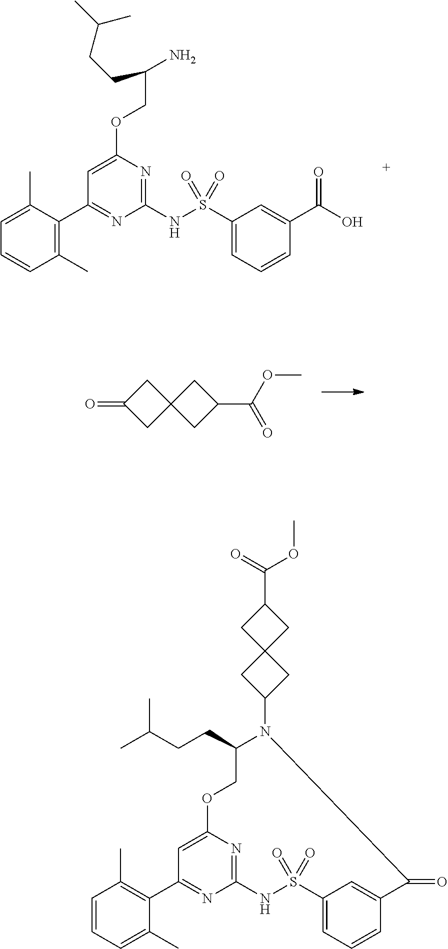 [SMILES: Cc1cccc(C)c1-c1cc(OC[C@H](N)CCC(C)C)nc(NS(=O)(=O)c2cccc(C(=O)O)c2)n1 ; COC(=O)C1CC2(CC(=O)C2)C1 ; COC(=O)C1CC2(C1)CC(N1C(=O)c3cccc(c3)S(=O)(=O)Nc3nc(cc(-c4c(C)cccc4C)n3)OC[C@H]1CCC(C)C)C2]

In a 4 mL vial, 3-[[4-[(2R)-2-amino-5-methyl-hexoxy]-6-(2,6-dimethylphenyl)pyrimidin-2-yl]sulfamoyl]benzoic acid (hydrochloride salt) (118 mg, 0.2149 mmol) was combined under nitrogen with methyl 2-oxospiro[3.3]heptane-6-carboxylate (64 mg, 0.3805 mmol) in anhydrous DCM (0.6 mL) and stirred for 5 minutes at room temperature. sodium triacetoxyborohydride (150 mg, 0.7077 mmol) was added and the mixture was stirred at room temperature for 3.5 h. The reaction mixture was partitioned between 1M HCl, brine and ethyl acetate. The layers were separated and the aqueous was extracted an additional three times with ethyl acetate. The combined organics were washed with brine, dried over sodium sulfate, and concentrated. The residue was dissolved in DMSO (2 mL). The solution was microfiltered through a syringe filter disc and purified by reverse phase preparative HPLC (C₁₈) using a gradient of acetonitrile in water (1 to 99% over 15 min) and HCl as a modifier. Evaporation gave 3-[[4-(2,6-dimethylphenyl)-6-[(2R)-2-[(6-methoxycarbonylspiro[3.3]heptan-2-yl)amino]-5-methyl-hexoxy]pyrimidin-2-yl]sulfamoyl]benzoic acid (hydrochloride salt) (77 mg, 51%) as a white solid. ESI-MS m/z calc. 664.2931, found 665.35 (M+1)⁺; Retention time: 1.32 minutes (LC method A).

A 20 mL vial was charged under nitrogen with [dimethylamino(triazolo[4,5-b]pyridin-3-yloxy)methylene]-dimethyl-ammonium (Phosphorus Hexafluoride Ion) (193 mg, 0.5076 mmol) (HATU), anhydrous DMF (5 mL) and DIEA (0.19 mL, 1.091 mmol). A solution of the intermediate above in DMF (1 mL) was added dropwise through syringe. The mixture was stirred at room temperature for 21 h. The mixture was concentrated under reduced pressure then partitioned between 1M HCl, brine and ethyl acetate. The layers were separated, and the aqueous was extracted an additional 2×with ethyl acetate. The combined organics were washed with brine, dried over sodium sulfate and concentrated. The resulting crude material was dissolved in DCM/MeOH and purified by flash chromatography on silica gel (12 g column) using a gradient of ethyl acetate (0 to 100% over 20 min) in hexanes. Evaporation of the solvents gave methyl 6-[(11R)-6-(2,6-dimethylphenyl)-11-(3-methylbutyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]spiro[3.3]heptane-2-carboxylate (27.7 mg, 20%) as a white solid (1:1 mixture of isomers). ESI-MS m/z calc. 646.28253, found 647.35 (M+1)⁺; Retention time: 2.0 minutes; second isomer, retention time 2.02 minutes (LC method A).

### Step 2: (11R)-6-(2,6-Dimethylphenyl)-12-[6-(2-hydroxypropan-2-yl)spiro[3.3]heptan-2-yl]-11-(3-methylbutyl)-9-oxa-2λ⁶-thia-3,5,12,19-tetraazatricyclo[12.3.1.14,8]nonadeca-1(17),4(19),5,7,14(18),15-hexaene-2,2,13-trione, more polar isomer peak 1 (Compound 4), and (11R)-6-(2,6-dimethylphenyl)-12-[6-(2-hydroxypropan-2-yl)spiro[3.3]heptan-2-yl]-11-(3-methylbutyl)-9-oxa-2λ⁶-thia-3,5,12,19-tetraazatricyclo[12.3.1.14,8]nonadeca-1(17),4(19),5,7,14(18),15-hexaene-2,2,13-trione, less polar isomer peak 2 (Compound 5)

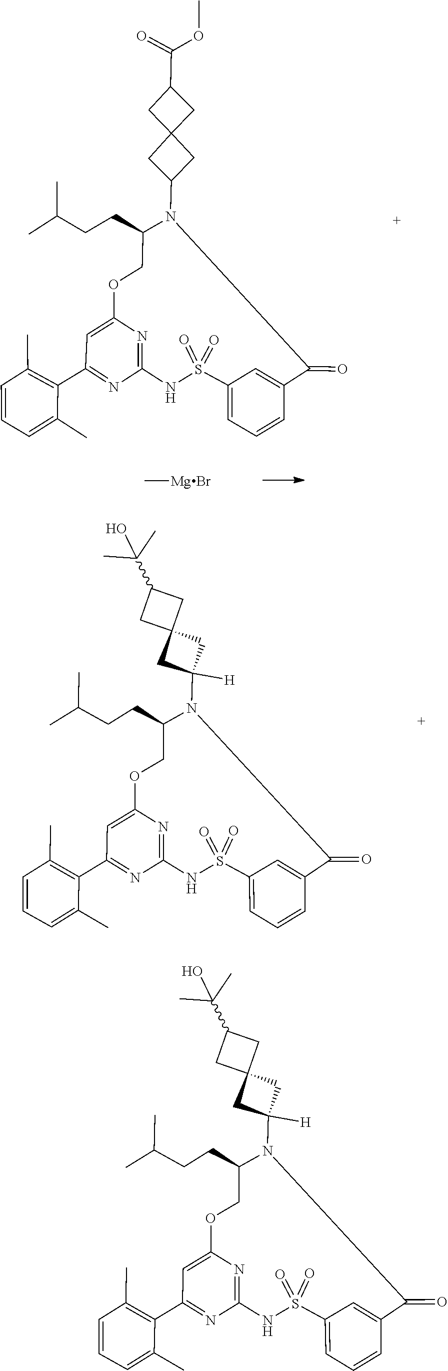

A 4 mL vial was charged under nitrogen with methyl 6-[(11R)-6-(2,6-dimethylphenyl)-11-(3-methylbutyl)-2,2,13-trioxo-9-oxa-2 λ6-thia-3,5,12,19-tetraazatricyclo[12.3.1.14,8]nonadeca-1(17),4(19), 5,7,14(18),15-hexaen-12-yl]spiro[3.3]heptane-2-carboxylate (27 mg, 0.04174 mmol) (isomer ratio 1:1), anhydrous THF (300 μL) and the solution was cooled down in an ice bath. Methyl Magnesium Bromide (0.050 mL of 3 M, 0.1500 mmol) (3M solution in diethyl ether) was added dropwise. The reaction mixture was stirred in the ice bath for 5 min, then it was stirred at room temperature for 4 h. The mixture was cooled down in ice and quenched by adding an aqueous saturated solution of ammonium chloride (5 drops) and DMSO (1 mL). The solution was microfiltered through a syringe filter disc and purified by reverse phase preparative HPLC (C₁₈) using a gradient of acetonitrile in water (0-60% over 20 min then 60-100% over 5 min) and HCl as a modifier, which resulted after evaporation in the isolation of two separated isomers: More polar isomer peak 1 (11R)-6-(2,6-dimethylphenyl)-12-[6-(2-hydroxypropan-2-yl)spiro[3.3]heptan-2-yl]-11-(3-methylbutyl)-9-oxa-2λ⁶-thia-3,5,12,19-tetraazatricyclo[12.3.1.14,8]nonadeca-1(17),4(19),5,7,14(18),15-hexaene-2,2,13-trione (10 mg, 74%). ESI-MS m/z calc. 646.3189, found 647.35 (M+1)⁺; Retention time: 1.96 minutes (LC method A). ¹H NMR (400 MHz, DMSO-d₆) δ 13.19-11.69 (broad m, 1H), 8.36 (s, 1H), 7.88 (s, 1H), 7.64 (s, 2H), 7.24 (d, J=7.8 Hz, 1H), 7.12 (d, J=7.5 Hz, 2H), 6.36 (s, 1H), 5.10 (dd, J=11.0, 3.9 Hz, 1H), 4.34 (t, J=11.2 Hz, 1H), 3.98 (s, 1H), 3.82 (p, J=8.6 Hz, 1H), 3.69-3.55 (m, 1H), 2.90 (t, J=9.6 Hz, 1H), 2.82 (t, J=9.8 Hz, 1H), 2.32-2.25 (m, 1H), 2.19-1.88 (m, 11H), 1.80 (t, J=9.5 Hz, 1H), 1.68-1.53 (m, 1H), 1.53-1.37 (m, 1H), 1.18-0.88 (m, 8H), 0.83-0.71 (m, 1H), 0.68 (d, J=6.4 Hz, 3H), 0.60 (d, J=6.4 Hz, 3H); and the less polar isomer peak 2 (11R)-6-(2,6-dimethylphenyl)-12-[6-(2-hydroxypropan-2-yl)spiro[3.3]heptan-2-yl]-11-(3-methylbutyl)-9-oxa-2λ⁶-thia-3,5,12,19-tetraazatricyclo[12.3.1.14,8]nonadeca-1(17),4(19),5,7,14(18),15-hexaene-2,2,13-trione (10.3 mg, 74%). ESI-MS m/z calc. 646.3189, found 647.35 (M+1)⁺; Retention time: 2.0 minutes (LC method A). ¹H NMR (400 MHz, DMSO-d₆) δ 13.45-11.43 (broad m, 1H), 8.36 (s, 1H), 7.88 (s, 1H), 7.65 (s, 2H), 7.32-7.19 (m, 1H), 7.12 (d, J=7.7 Hz, 2H), 6.36 (s, 1H), 5.09 (dd, J=10.8, 3.8 Hz, 1H), 4.32 (t, J=11.2 Hz, 1H), 3.99 (s, 1H), 3.82 (p, J=8.6 Hz, 1H), 3.59 (d, J=12.0 Hz, 1H), 2.92 (t, J=9.5 Hz, 1H), 2.80 (t, J=9.8 Hz, 1H), 2.32-2.26 (m, 1H), 2.20-1.88 (m, 11H), 1.85-1.74 (m, 1H), 1.70-1.53 (m, 1H), 1.53-1.39 (m, 1H), 1.14-0.90 (m, 8H), 0.86-0.73 (m, 1H), 0.68 (d, J=6.3 Hz, 3H), 0.61 (d, J=6.3 Hz, 3H).

### Example 5: Preparation of Compound 6

### Step 1: Iodo-1-(2-methoxyethyl)pyrazole

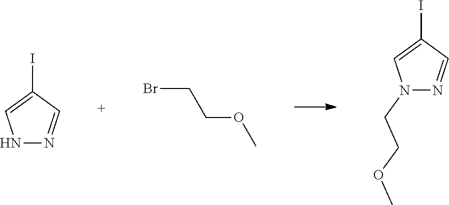

4-Iodo-1H-pyrazole (2 g, 10.31 mmol) was combined with cesium carbonate (5.1 g, 15.65 mmol) in anhydrous acetonitrile (15 mL). 1-Bromo-2-methoxy-ethane (1.15 mL, 12.24 mmol) was added and the reaction mixture was stirred vigorously at room temperature for 18 hours. The reaction mixture was then filtered through Celite, eluting with acetonitrile. The filtrate was concentrated, then dissolved in diethyl ether and filtered through Celite a second time. The filtrate was again concentrated to give as a slightly yellow oil, 4-iodo-1-(2-methoxyethyl)pyrazole (2.2 g, 85%) ESI-MS m/z calc. 251.97595, found 253.3 (M+1)⁺; Retention time: 0.41 minutes (LC method D). ¹H NMR (400 MHz, Chloroform-d) δ 7.53 (s, 1H), 7.51 (s, 1H), 4.29 (t, J=5.1 Hz, 2H), 3.71 (t, J=5.1 Hz, 2H), 3.33 (s, 3H).

### Step 2: (2R)-2-[[1-(2-Methoxyethyl)pyrazol-4-yl]amino]-5-methyl-hexan-1-ol

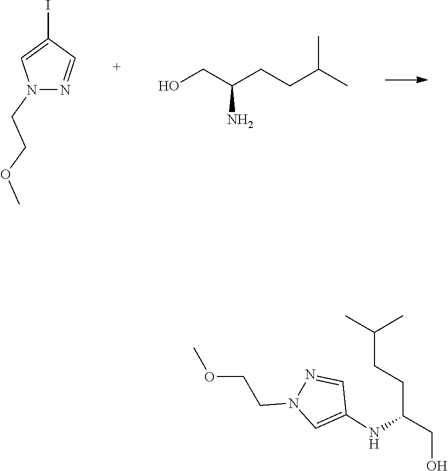

4-Iodo-1-(2-methoxyethyl)pyrazole (approximately 150.3 mg, 0.5965 mmol) was combined with the (2R)-2-amino-5-methyl-hexan-1-ol (hydrochloride salt) (100 mg, 0.5964 mmol), CuI (approximately 11.36 mg, 0.05965 mmol), and NaOH (approximately 95.43 mg, 2.386 mmol) (ground with mortar and pestle) in a screw cap vial, which was then purged with nitrogen. DMSO (0.3 mL) and water (0.15 mL) were added and the reaction mixture was stirred at 90° C. for 16 hours. After cooling to room temperature, the reaction mixture was diluted with methanol and filtered. The filtrate was concentrated by rotary evaporation and the resulting residue was dissolved in 1:1 DMSO/methanol, filtered a second time and purified by reverse phase HPLC (1-50% ACN in water, HCl modifier, 15 min run) to give the indicated (2R)-2-[[1-(2-methoxyethyl)pyrazol-4-yl]amino]-5-methyl-hexan-1-ol (hydrochloride salt) (115 mg, 66%) upon drying. ESI-MS m/z calc. 255.19467, found 256.6 (M+1)⁺; Retention time: 0.32 minutes; LC method D.

### Step 3: (11R)-6-(2,6-Dimethylphenyl)-11-isopentyl-12-[1-(2-methoxyethyl)pyrazol-4-yl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 6)

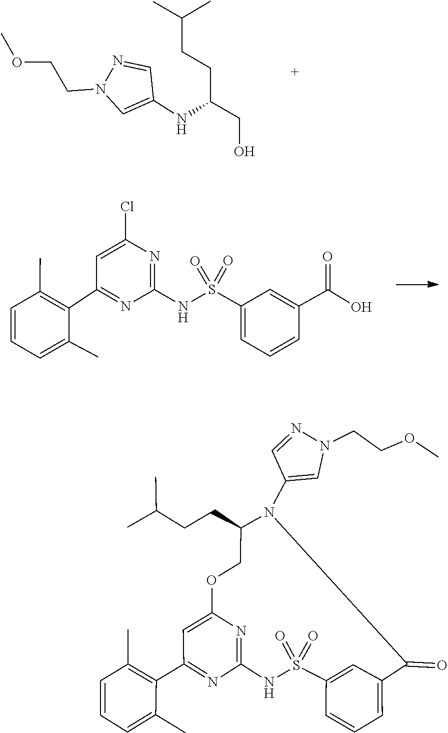 [SMILES: COCCn1cc(N[C@@H](CO)CCC(C)C)cn1 ; Cc1cccc(C)c1-c1cc(Cl)nc(NS(=O)(=O)c2cccc(C(=O)O)c2)n1 ; COCCn1cc(N2C(=O)c3cccc(c3)S(=O)(=O)Nc3nc(cc(-c4c(C)cccc4C)n3)OC[C@H]2CCC(C)C)cn1]

(2R)-2-[[1-(2-Methoxyethyl)pyrazol-4-yl]amino]-5-methyl-hexan-1-ol (hydrochloride salt) (115 mg, 0.3941 mmol) was combined with 3-[[4-chloro-6-(2,6-dimethylphenyl)pyrimidin-2-yl]sulfamoyl]benzoic acid (approximately 126.7 mg, 0.3032 mmol) in THE (0.75 mL) and stirred until the solids had mostly dissolved/become a suspension. Sodium tert-butoxide (approximately 174.9 mg, 1.820 mmol) was added and the reaction briefly became slightly warm. Stirring was continued for 15 minutes with no external heating. The reaction mixture was then partitioned between 1M HCl and ethyl acetate. The layers were separated, and the aqueous was extracted an additional 4×with ethyl acetate. The combined organics were washed with brine, dried over sodium sulfate, and concentrated. The resulting crude material was dissolved in 1:1 DMSO/methanol, filtered, and purified by reverse phase HPLC (1-60 ACN in water, HCl modifier, 15 min run) to give the SNAr product. The product was dissolved in DMF (8 mL) and NN (approximately 122.7 mg, 133.4 μL, 1.213 mmol) was added. The reaction mixture was cooled to 0° C. and CDMT (approximately 79.87 mg, 0.4549 mmol) was added. The reaction was allowed to warm to room temperature as the ice melted and stirred for 48 hours. The reaction mixture was quenched with several drops of water, partially concentrated, diluted with 1:1 DMSO/methanol, filtered, and purified by reverse phase HPLC (1-70% ACN in water, HCl modifier, 30 min run) to give (11R)-6-(2,6-dimethylphenyl)-11-isopentyl-12-[1-(2-methoxyethyl)pyrazol-4-yl]-2,2-dioxo-9-oxa-2λ⁶-thia-3,5,12,19-tetrazatricyclo[12.3.1.14,8]nonadeca-1(18),4(19),5,7,14,16-hexaen-13-one (30 mg, 16%). ESI-MS m/z calc. 618.26245, found 619.5 (M+1)⁺; Retention time: 1.65 minutes; LC method A.

### Example 6: Preparation of Compound 7

### Step 1: (2R)-5-Methyl-2-[(1-methylpyrazol-4-yl)amino]hexan-1-ol

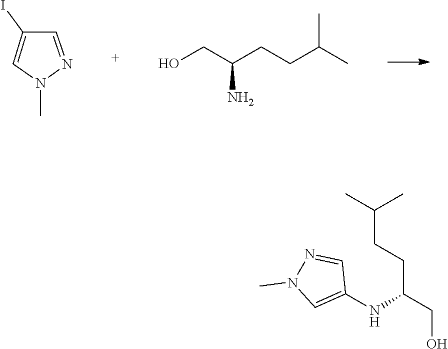

The 4-iodo-1-methyl-pyrazole (approximately 124.1 mg, 0.5965 mmol) was combined with the (2R)-2-amino-5-methyl-hexan-1-ol (hydrochloride salt) (100 mg, 0.5964 mmol), CuI (approximately 11.36 mg, 0.05965 mmol), and NaOH (approximately 95.43 mg, 2.386 mmol) (ground with mortar and pestle) in a screw cap vial, which was then purged with nitrogen. DMSO (0.3 mL) and water (0.15 mL) were added and the reaction mixture was stirred at 90° C. for 16 hours. After cooling to room temperature, the reaction mixture was diluted with methanol and filtered. The filtrate was concentrated by rotary evaporation and the resulting residue was dissolved in 1:1 DMSO/methanol, filtered a second time and purified by reverse phase HPLC (1-50% ACN in water, HCl modifier, 15 min run) to give the indicated (2R)-5-methyl-2-[(1-methylpyrazol-4-yl)amino]hexan-1-ol (hydrochloride salt) (86 mg, 58%) upon drying. ESI-MS m/z calc. 211.16846, found 212.6 (M+1)⁺; Retention time: 0.29 minutes; LC method D.

### Step 2: (11R)-6-(2,6-Dimethylphenyl)-11-isopentyl-12-(1-methylpyrazol-4-yl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 7)

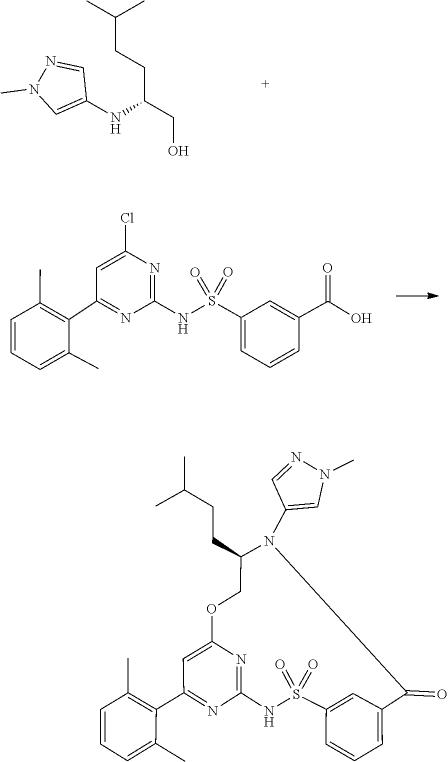 [SMILES: CC(C)CC[C@H](CO)Nc1cnn(C)c1 ; Cc1cccc(C)c1-c1cc(Cl)nc(NS(=O)(=O)c2cccc(C(=O)O)c2)n1 ; Cc1cccc(C)c1-c1cc2nc(n1)NS(=O)(=O)c1cccc(c1)C(=O)N(c1cnn(C)c1)[C@H](CCC(C)C)CO2]

(2R)-5-methyl-2-[(1-methylpyrazol-4-yl)amino]hexan-1-ol (hydrochloride salt) (86 mg, 0.3471 mmol) was combined with 3-[[4-chloro-6-(2,6-dimethylphenyl)pyrimidin-2-yl]sulfamoyl]benzoic acid (approximately 111.6 mg, 0.2670 mmol) in THE (0.75 mL) and stirred until the solids had mostly dissolved. Sodium tert-butoxide (approximately 154.0 mg, 1.602 mmol) was added and the reaction briefly became slightly warm. Stirring was continued for 15 minutes with no external heating. The reaction mixture was then partitioned between 1M HCl and ethyl acetate. The layers were separated, and the aqueous was extracted an additional 4 with ethyl acetate. The combined organics were washed with brine, dried over sodium sulfate, and concentrated. The resulting crude material was dissolved in 1:1 DMSO/methanol, filtered, and purified by reverse phase HPLC (1-60 ACN in water, HCl modifier, 15 min run) to give the SNAr product. The product was dissolved in DMF (8 mL) and NN (approximately 162.0 mg, 176.1 μL, 1.602 mmol) was added. The reaction mixture was cooled to 0° C. and CDMT (approximately 70.32 mg, 0.4005 mmol) was added. The reaction was allowed to warm to room temperature as the ice melted and stirred for 48 hours. The reaction mixture was quenched with several drops of water, partially concentrated, diluted with 1:1 DMSO/methanol, filtered, and purified by reverse phase HPLC (1-70% ACN in water, HCl modifier, 30 min run) to give (11R)-6-(2,6-dimethylphenyl)-11-isopentyl-12-(1-methylpyrazol-4-yl)-2,2-dioxo-9-oxa-2λ⁶-thia-3,5,12,19-tetrazatricyclo[12.3.1.14,8]nonadeca-1(18),4(19),5,7,14,16-hexaen-13-one (29.0 mg, 19%). ESI-MS m/z calc. 574.2362, found 575.5 (M+1)⁺; Retention time: 1.61 minutes; LC method A.

### Example 7: Preparation of Compound 8 and Compound 9

### Step 1: Methyl 2-(tert-butoxycarbonylamino)-5,5-dimethyl-hex-2-enoate

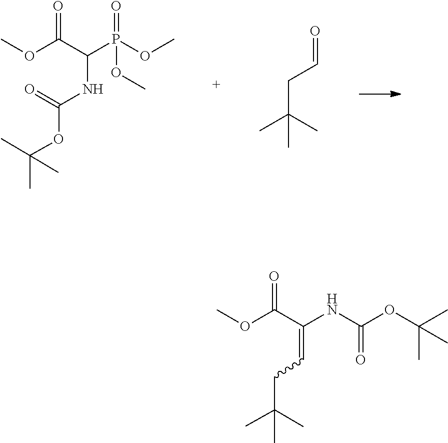

To a stirred solution of methyl 2-(tert-butoxycarbonylamino)-2-dimethoxyphosphoryl-acetate (2.86 g, 9.6218 mmol) and DBU (1.4252 g, 1.4 mL, 9.3617 mmol) in DCM (20 mL) was added 3,3-dimethylbutyraldehyde (997.50 mg, 1.25 mL, 8.7358 mmol). The reaction mixture was stirred at room temperature for 16 h. Aqueous HCl (1 N) (25 mL) was added and the phases were separated. The aqueous layer was washed with DCM (2×20 mL). The combined organic layers were dried over magnesium sulfate, filtered and concentrated under reduced pressure. The crude residue was purified by chromatography on a 40 g silica gel cartridge using a gradient of 0-30% EtOAc in heptanes to afford methyl 2-(tert-butoxycarbonylamino)-5,5-dimethyl-hex-2-enoate (2.305 g, 97%) as a clear oil that crystallized to a white solid. ESI-MS m/z calc. 271.1784, found 216.4 (M-55)⁺; Retention time: 1.91 minutes LC method X. ¹H NMR (400 MHz, CDCl₃) δ 6.67 (t, J=7.6 Hz, 1H), 5.86 (br. s., 1H), 3.79 (s, 3H), 2.12 (d, J=7.6 Hz, 2H), 1.47 (s, 9H), 0.96 (s, 9H).

### Step 2: Methyl (2R)-2-(tert-butoxycarbonylamino)-5,5-dimethyl-hexanoate

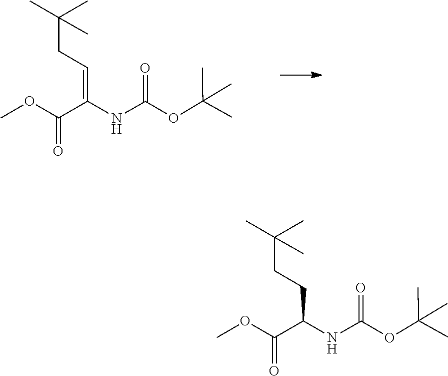 [SMILES: COC(=O)/C(=C\CC(C)(C)C)NC(=O)OC(C)(C)C ; COC(=O)[C@@H](CCC(C)(C)C)NC(=O)OC(C)(C)C]

To a solution of methyl (E)-2-(tert-butoxycarbonylamino)-4,5,5-trimethyl-hex-2-enoate (2 g, 7.0082 mmol) in ethanol (27 mL) and 1,4-dioxane (9 mL) was bubbled nitrogen for 5 min. Then, 1,2-bis[(2R,5R)-2,5-diethylphospholano]benzene(1,5-cyclooctadiene)rhodium(I) trifluoromethanesulfonate (51 mg, 0.0706 mmol) was added and the mixture was put in an ultrasound bath for 5 min. under nitrogen. The reaction mixture was hydrogenated under 50 psi (3.5 bar) of hydrogen pressure and at room temperature for 16 h. Silica gel was added to the reaction mixture and it was evaporated to dryness. The product was purified by chromatography on a 40 g silica gel cartridge using a gradient of 0-30% EtOAc in heptanes to afford methyl (2R)-2-(tert-butoxycarbonylamino)-5,5-dimethyl-hexanoate (1.91 g, 100%). ESI-MS m/z calc. 273.194, found 218.4 (M-55)⁺; Retention time: 1.96 minutes, LC method X. ¹H NMR (400 MHz, CDCl₃) δ 5.08-4.88 (m, 1H), 4.36-4.21 (m, 1H), 3.75 (s, 3H), 1.85-1.74 (m, 1H), 1.67-1.59 (m, 1H), 1.45 (s, 9H), 1.26-1.16 (m, 2H), 0.87 (s, 9H).

### Step 3: tert-Butyl N-[(1R)-1-(hydroxymethyl)-4,4-dimethyl-pentyl]carbamate

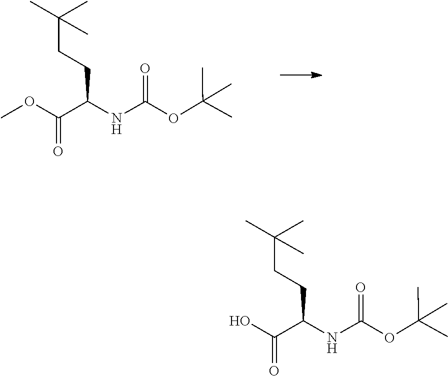 [SMILES: COC(=O)[C@@H](CCC(C)(C)C)NC(=O)OC(C)(C)C ; CC(C)(C)CC[C@@H](NC(=O)OC(C)(C)C)C(=O)O]

To a solution of methyl (2R)-2-(tert-butoxycarbonylamino)-5,5-dimethyl-hexanoate (1.9 g, 6.9503 mmol) in THE (20 mL) was added LiBH4 (2 M solution in THF) (8.8 mL of 2 M, 17.600 mmol). The reaction mixture was stirred at room temperature for 2.5 h. The reaction mixture was then poured slowly over a saturated aqueous solution of ammonium chloride (50 mL) at 0° C. (strong evolution of gas, but no exotherm). The product was extracted with EtOAc (3×50 mL). The combined organic layers were washed with brine (50 mL), dried over magnesium sulfate, filtered and concentrated under reduced pressure to afford crude product tert-butyl N-[(1R)-1-(hydroxymethyl)-4,4-dimethyl-pentyl]carbamate (1.725 g, 101%) as a clear oil. ¹H NMR. ESI-MS m/z calc. 245.1991, found 190.2 (M-55)⁺; Retention time: 1.81 minutes. ¹H NMR (400 MHz, CDCl₃) δ 4.65-4.51 (m, 1H), 3.74-3.65 (m, 1H), 3.62-3.51 (m, 2H), 2.35 (br. s., 1H), 1.46 (s, 9H), 1.42-1.17 (m, 4H), 0.89 (s, 9H). LC method X.

### Step 4:(2R)-2-Amino-5,5-dimethyl-hexan-1-ol

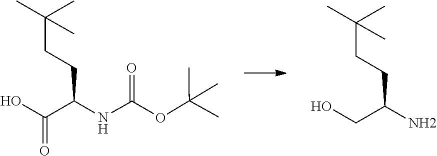

To a solution of tert-butyl N-[(1R)-1-(hydroxymethyl)-4,4-dimethyl-pentyl]carbamate (1.72 g, 7.0102 mmol) in 1,4-dioxane (9 mL) was added hydrogen chloride (4 N in 1,4-dioxane) (9 mL of 4 M, 36.000 mmol). The reaction mixture was stirred at room temperature for 16 h. The mixture was evaporated to give (2R)-2-amino-5,5-dimethyl-hexan-1-ol (hydrochloride salt) (1.19 g, 93%) as a white solid. ¹H NMR (400 MHz, DMSO-d₆) δ 7.81 (br. s., 3H), 5.26 (t, J=4.9 Hz, 1H), 3.58 (dt, J=11.5, 4.0 Hz, 1H), 3.47-3.37 (m, 1H), 2.98 (br. s., 1H), 1.53-1.41 (m, 2H), 1.26-1.14 (m, 2H), 0.87 (s, 9H). ESI-MS m/z calc. 145.14667, found 146.4 (M+1)⁺; Retention time: 1.05 minutes; LC method X.

### Step 5: 3-[[4-[(2R)-2-Amino-5,5-dimethyl-hexoxy]-6-(2,6-dimethylphenyl)pyrimidin-2-yl]sulfamoyl]benzoic acid

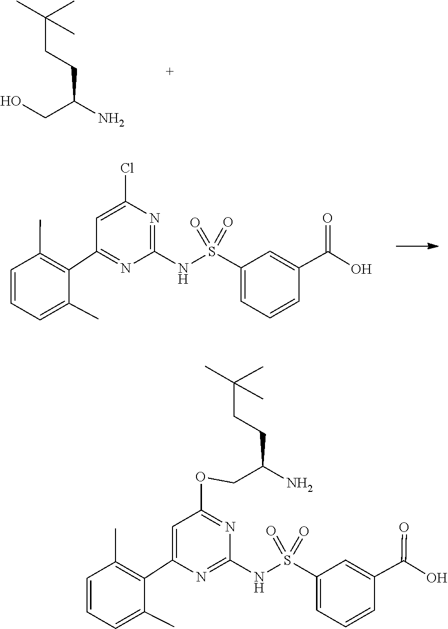

To a solution of 3-[[4-chloro-6-(2,6-dimethylphenyl)pyrimidin-2-yl]sulfamoyl]benzoic acid (hydrochloride salt) (2.71 g, 5.9649 mmol) and (2R)-2-amino-5,5-dimethyl-hexan-1-ol (hydrochloride salt) (1.19 g, 6.5491 mmol) in DMF (15 mL) maintained at 15° C. with a water bath was added sodium tert-butoxide (2.87 g, 29.864 mmol) and the mixture was stirred at room temperature for 1 h. 2-MeTHF (50 mL) was added followed by 1 N aqueous HCl (50 mL). The phases were separated, and the aqueous phase was washed with 2-MeTHF (4×50 mL). The combined organic layers were washed with 15% brine (2×50 mL), dried over magnesium sulfate, filtered and concentrated under reduced pressure. The crude foam (4 g, 119%) was triturated in EtOAc (50 mL) under stirring for 16 h. The solid partially dissolved after 16 h of stirring. The solvent was evaporated to dryness and the residue was then triturated in 1:1 heptanes/EtOAc (50 mL) for 1 h. The solid was filtered on a Buchner funnel and rinsed with 1:1 heptanes/EtOAc (50 mL). The solid was dried in vacuo to provide the product (3.3 g, 99%) as an off-white solid. The product was further purified by reverse phase chromatography on a 120 g C₁₈ cartridge using a gradient of 10-100% MeOH in water (with 0.1% HCl) to afford after lyophilization in water (15 mL) and MeCN (10 mL) 3-[[4-[(2R)-2-amino-5,5-dimethyl-hexoxy]-6-(2,6-dimethylphenyl)pyrimidin-2-yl]sulfamoyl]benzoic acid (hydrochloride salt) (2.37 g, 70%) as a pale pink solid. ESI-MS m/z calc. 526.225, found 527.2 (M+1)⁺; Retention time: 2.5 minutes. ¹H NMR (400 MHz, DMSO-d₆) δ 14.11-12.43 (m, 2H), 8.45 (s, 1H), 8.40-8.07 (m, 5H), 7.69 (t, J=7.8 Hz, 1H), 7.31-7.21 (m, 1H), 7.13 (d, J=7.8 Hz, 2H), 6.31 (br. s., 1H), 4.43-4.35 (m, 1H), 4.34-4.25 (m, 1H), 3.53-3.42 (m, 1H), 2.00 (br. s., 6H), 1.67-1.50 (m, 2H), 1.37-1.25 (m, 1H), 1.24-1.12 (m, 1H), 0.86 (s, 9H). LC method Y.

### Step 6: 3-[[4-(2,6-Dimethylphenyl)-6-[(2R)-2-[(6-methoxycarbonylspiro[3.3]heptan-2-yl)amino]-5,5-dimethyl-hexoxy]pyrimidin-2-yl]sulfamoyl]benzoic acid

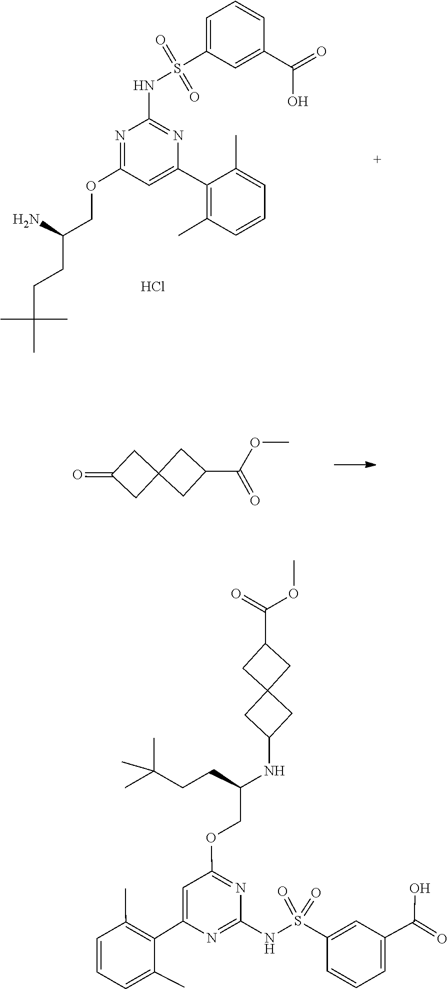 [SMILES: Cc1cccc(C)c1-c1cc(OC[C@H](N)CCC(C)(C)C)nc(NS(=O)(=O)c2cccc(C(=O)O)c2)n1 ; Cl ; COC(=O)C1CC2(CC(=O)C2)C1 ; COC(=O)C1CC2(CC(N[C@H](CCC(C)(C)C)COc3cc(-c4c(C)cccc4C)nc(NS(=O)(=O)c4cccc(C(=O)O)c4)n3)C2)C1]

In a 20 mL vial, 3-[[4-[(2R)-2-amino-5,5-dimethyl-hexoxy]-6-(2,6-dimethylphenyl)pyrimidin-2-yl]sulfamoyl]benzoic acid (hydrochloride salt) (298 mg, 0.4970 mmol) was combined under nitrogen with methyl 2-oxospiro[3.3]heptane-6-carboxylate (135 mg, 0.8027 mmol) in anhydrous DCM (1.5 mL) and stirred for 5 minutes at room temperature. sodium triacetoxyborohydride (347 mg, 1.637 mmol) was added and the mixture was stirred at room temperature for 4 h. The reaction mixture was partitioned between 1M HCl, brine and ethyl acetate. The layers were separated and the aqueous was extracted an additional three times with ethyl acetate. The combined organics were dried over sodium sulfate, and concentrated. The residue was triturated in diethylether and the resulting solid was filtered and dried to give crude 3-[[4-(2,6-dimethylphenyl)-6-[(2R)-2-[(6-methoxycarbonylspiro[3.3]heptan-2-yl)amino]-5,5-dimethyl-hexoxy]pyrimidin-2-yl]sulfamoyl]benzoic acid (hydrochloride salt) (243 mg, 68%) as a white solid. The product was used for the next step without any further purification. ESI-MS m/z calc. 678.3087, found 679.34 (M+1)⁺; Retention time: 1.38 minutes. LC method A.

### Step 7: Methyl 2-[(11R)-11-(3,3-dimethylbutyl)-6-(2,6-dimethylphenyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]spiro[3.3]heptane-6-carboxylate

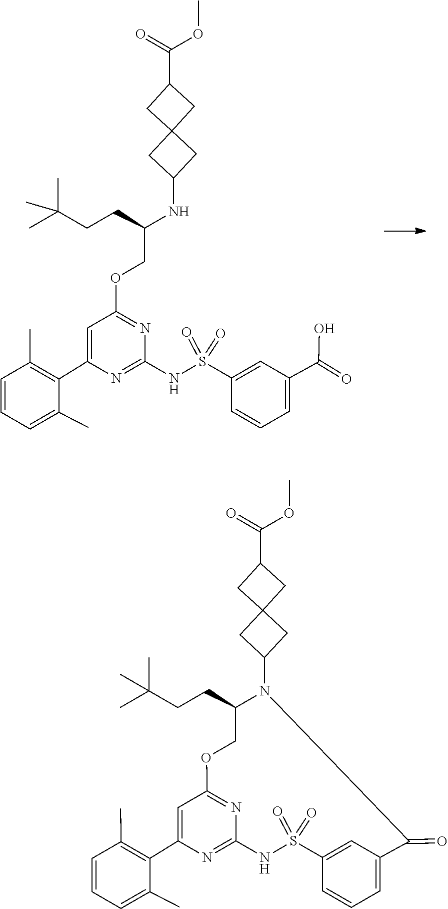 [SMILES: COC(=O)C1CC2(CC(N[C@H](CCC(C)(C)C)COc3cc(-c4c(C)cccc4C)nc(NS(=O)(=O)c4cccc(C(=O)O)c4)n3)C2)C1 ; COC(=O)C1CC2(C1)CC(N1C(=O)c3cccc(c3)S(=O)(=O)Nc3nc(cc(-c4c(C)cccc4C)n3)OC[C@H]1CCC(C)(C)C)C2]

3-[[4-(2,6-dimethylphenyl)-6-[(2R)-2-[(6-methoxycarbonylspiro[3.3]heptan-2-yl)amino]-5,5-dimethyl-hexoxy]pyrimidin-2-yl]sulfamoyl]benzoic acid (hydrochloride salt) (243 mg, 0.3397 mmol) was dissolved in DMF (2.4 mL). N-methylmorpholine (57 μL, 0.5185 mmol) was added, and the solution was cooled to 0° C. before the addition of 2-chloro-4,6-dimethoxy-1,3,5-triazine (78 mg, 0.4443 mmol). The reaction mixture was allowed to warm to room temperature, stirred for 3 hours and stored overnight in a 4° C. refrigerator. The reaction mixture was then diluted with EtOAc (50 mL) and washed with aqueous HCl (1×50 mL). The aqueous layer was further extracted with EtOAc (2×50 mL). All organic layers were combined, dried over sodium sulfate, filtered and concentrated under reduced pressure. The crude product was purified by chromatography on a 12 gram silica gel column eluting with a 0-50% EtOAc/hexane gradient over 40 minutes to give methyl 2-[(11R)-11-(3,3-dimethylbutyl)-6-(2,6-dimethylphenyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]spiro[3.3]heptane-6-carboxylate (183 mg, 82%) was obtained as a white solid. ESI-MS m/z calc. 660.29816, found 661.3 (M+1)⁺; Retention time: 2.11 minutes; the second diastereomer had retention time of 2.13 minutes. LC method A.

### Step 8: (11R)-11-(3,3-Dimethylbutyl)-6-(2,6-dimethylphenyl)-12-[6-(1-hydroxy-1-methyl-ethyl)spiro[3.3]heptan-2-yl]-2,2-dioxo-9-oxa-2λ⁶-thia-3,5,12,19-tetrazatricyclo[12.3.1.14,8]nonadeca-1(18),4(19),5,7,14,16-hexaen-13-one, less polar isomer (Compound 8), and (11R)-11-(3,3-dimethylbutyl)-6-(2,6-dimethylphenyl)-12-[6-(1-hydroxy-1-methyl-ethyl)spiro[3.3]heptan-2-yl]-2,2-dioxo-9-oxa-2λ⁶-thia-3,5,12,19-tetrazatricyclo[12.3.1.14,8]nonadeca-1(18),4(19),5,7,14,16-hexaen-13-one, more polar isomer (Compound 9)

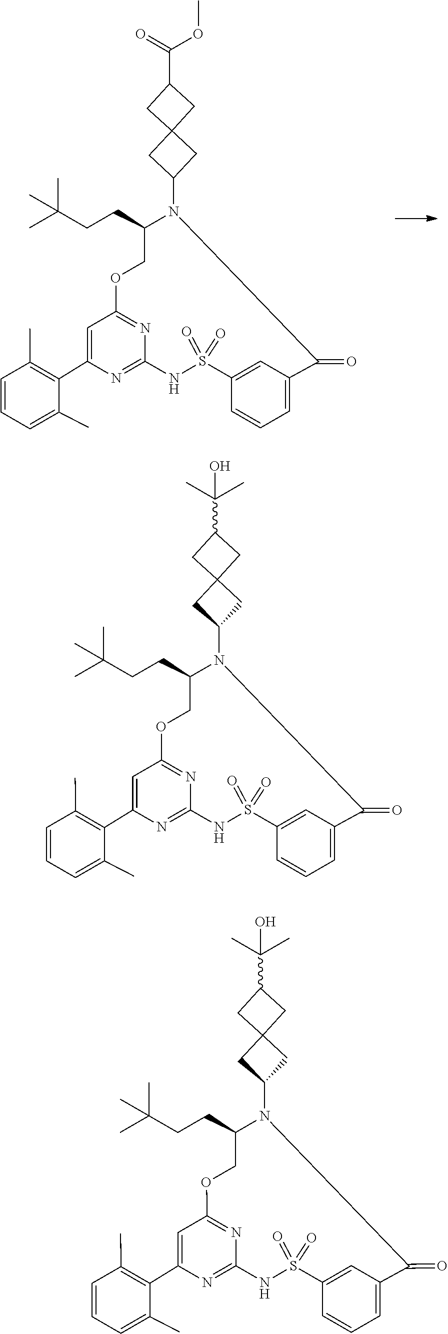

Methyl 2-[(11R)-11-(3,3-dimethylbutyl)-6-(2,6-dimethylphenyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]spiro[3.3]heptane-6-carboxylate (187 mg, 0.2830 mmol) was dissolved in THE (2.00 mL). At 0° C. under nitrogen, bromo(methyl)magnesium (340 μL of 3 M, 1.020 mmol) in diethyl ether was slowly added. The reaction mixture was stirred at 0° C. for 5 minutes, allowed to warm to room temperature and then allowed to stir for 2 hours. The reaction mixture was diluted with methanol (1 mL) and DMSO (3 mL) and purified by UV-triggered reverse-phase HPLC eluting with a 35-50% acetonitrile/water gradient over 30 minutes with 5 mM HCl acid modifier. Less polar isomer, (11R)-11-(3,3-dimethylbutyl)-6-(2,6-dimethylphenyl)-12-[6-(1-hydroxy-1-methyl-ethyl)spiro[3.3]heptan-2-yl]-2,2-dioxo-9-oxa-2λ⁶-thia-3,5,12,19-tetrazatricyclo[12.3.1.14,8]nonadeca-1(18),4(19),5,7,14,16-hexaen-13-one (63.3 mg, 68%) was obtained as a white solid. ESI-MS m/z calc. 660.33453, found 661.3 (M+1)⁺; Retention time: 2.99 minutes (LC method I); More polar isomer, (11R)-11-(3,3-dimethylbutyl)-6-(2,6-dimethylphenyl)-12-[6-(1-hydroxy-1-methyl-ethyl)spiro[3.3]heptan-2-yl]-2,2-dioxo-9-oxa-2λ⁶-thia-3,5,12,19-tetrazatricyclo[12.3.1.14,8]nonadeca-1(18),4(19),5,7,14,16-hexaen-13-one (27.3 mg, 29%) was obtained as a white solid. ESI-MS m/z calc. 660.33453, found 661.3 (M+1)⁺; Retention time: 2.93 minutes, LC method I.

### Example 8: Preparation of Compound 10

### Step 1: (4-Fluoro-2,6-dimethyl-phenyl)boronic acid

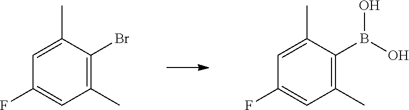

To a solution of 2-bromo-5-fluoro-1,3-dimetyl-benzene 30 g, 147.75 mmol) in THE (300.00 mL) was added n-BuLi (74 mL of 2.5 M, 185.00 mmol) dropwise at −78° C. The solution was stirred for 2 h at −78° C. before Trimethyl Borate (35.319 g, 38.6 mL, 339.89 mmol) was added dropwise. The solution was stirred at room temperature overnight before being quenched with 1M HCl (300 mL) and water (200 mL). This solution was stirred for 1 h then diluted with diethyl ether (200 mL) and washed with water (200 mL). The organic layer was then separated, dried over sodium sulfate and concentrated. The solid was then triturated with hexanes (2×100 mL) to give (4-fluoro-2,6-dimethyl-phenyl)boronic acid (11.154 g, 38%) as a white solid. ¹H NMR (500 MHz, DMSO-d₆) δ 8.18 (s, 1H), 6.76 (d, J=10.4 Hz, 2H), 2.28 (s, 6H).

### Step 2: tert-Butyl N-tert-butoxycarbonyl-N-[4-chloro-6-(4-fluoro-2,6-dimethyl-phenyl)pyrimidin-2-yl]carbamate

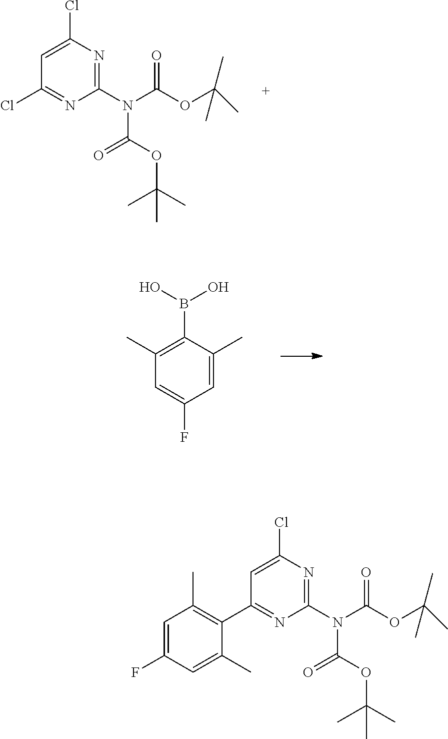

To a solution of tert-butyl N-tert-butoxycarbonyl-N-(4,6-dichloropyrimidin-2-yl)carbamate (12.43 g, 34.127 mmol) dissolved in DME (87 mL) and water (12 mL) was added (4-fluoro-2,6-dimethyl-phenyl)boronic acid (7.45 g, 44.352 mmol) and cesium carbonate (28.9 g, 88.700 mmol) at room temperature. The solution was stirred for 10 min while being bubbled with a nitrogen stream. Then Pd(dppf)Cl₂ (2.5 g, 3.4167 mmol) was added to the solution and heated to 80° C. for 2 h. The solution was cooled to room temperature before being diluted with water (100 mL) and extracted with ethyl acetate (2×100 mL). The combined organic layer was washed with brine (200 mL) and dried over sodium sulfate before being concentrated in vacuum. The organic residue was purified by silica gel chromatography eluting 0-20% ethyl acetate-hexanes to give tert-butyl N-tert-butoxycarbonyl-N-[4-chloro-6-(4-fluoro-2,6-dimethyl-phenyl)pyrimidin-2-yl]carbamate (9.05 g, 59%). ESI-MS m/z calc. 451.1674, found 452.1 (M+1)⁺; Retention time: 4.11 minutes, LC method T.

### Step 3: 4-Chloro-6-(4-fluoro-2,6-dimethyl-phenyl)pyrimidin-2-amine

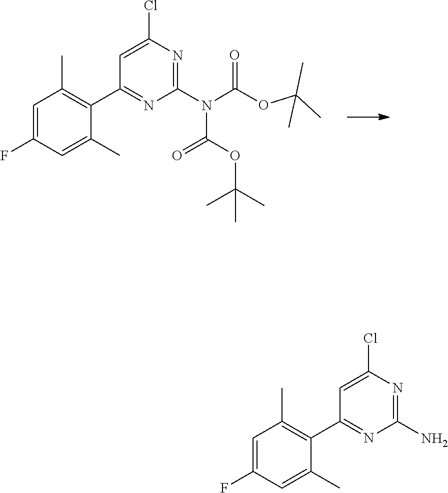 [SMILES: Cc1cc(F)cc(C)c1-c1cc(Cl)nc(N(C(=O)OC(C)(C)C)C(=O)OC(C)(C)C)n1 ; Cc1cc(F)cc(C)c1-c1cc(Cl)nc(N)n1]

To a solution of tert-butyl N-tert-butoxycarbonyl-N-[4-chloro-6-(4-fluoro-2,6-dimethyl-phenyl)pyrimidin-2-yl]carbamate (9.05 g, 20.026 mmol) in DCM (100 mL) was added TFA (29.600 g, 20 mL, 259.60 mmol) and the reaction was stirred for 2 h at room temperature. Volatiles were removed under vacuum and the residue was taken up in sodium bicarbonate (100 mL) and extracted with ethyl acetate (3×100 mL) and washed with brine (100 mL). The organic layer was dried over sodium sulfate and concentrated to give 4-chloro-6-(4-fluoro-2,6-dimethyl-phenyl)pyrimidin-2-amine (5.59 g, 111%) as a white foam solid. ESI-MS m/z calc. 251.0626, found 252.3 (M+1)⁺; Retention time: 2.29 minutes. ¹H NMR (500 MHz, DMSO-d₆) δ 7.24 (s, 2H), 6.98 (d, J=9.8 Hz, 2H), 6.65 (s, 1H), 2.08 (s, 6H)., LC method T.

### Step 4: Methyl 3-[[4-chloro-6-(4-fluoro-2,6-dimethyl-phenyl)pyrimidin-2-yl]sulfamoyl]benzoate

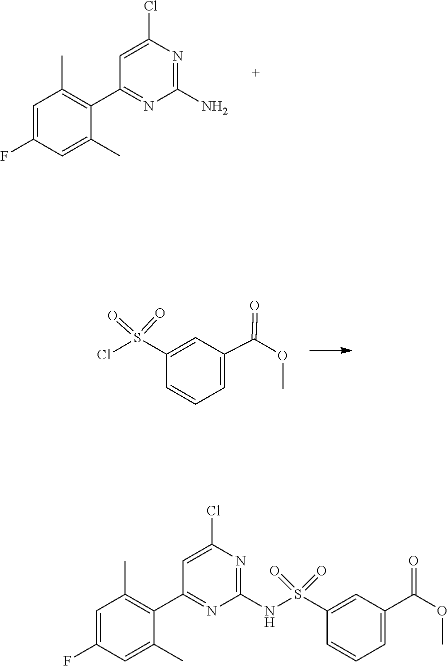 [SMILES: Cc1cc(F)cc(C)c1-c1cc(Cl)nc(N)n1 ; COC(=O)c1cccc(S(=O)(=O)Cl)c1 ; COC(=O)c1cccc(S(=O)(=O)Nc2nc(Cl)cc(-c3c(C)cc(F)cc3C)n2)c1]

To a solution of 4-chloro-6-(4-fluoro-2,6-dimethyl-phenyl)pyrimidin-2-amine (2.78 g, 11.045 mmol) in THE (40 mL) at 0° C. was added methyl 3-chlorosulfonylbenzoate (4.32 g, 18.410 mmol). Then Lithium tert-amoxide (6.1320 g, 21 mL of 40% w/w, 26.071 mmol) was added to the solution dropwise keeping the temperature below 5° C. The solution was allowed to warm to room temperature while it stirred overnight. The solution was acidified with 2M HCl (50 mL) and extracted with ethyl acetate (100 mL). The organic layer was washed with brine (200 mL) and dried over sodium sulfate. The organic layer was then concentrated in vacuum and purified using silica gel chromatography eluting 0-40% hexanes-ethyl acetate to give methyl 3-[[4-chloro-6-(4-fluoro-2,6-dimethyl-phenyl)pyrimidin-2-yl]sulfamoyl]benzoate (792 mg, 16%). ESI-MS m/z calc. 449.0612, found 450.0 (M+1)⁺; Retention time: 3.49 minutes, LC method T.

### Step 5: 3-[[4-Chloro-6-(4-fluoro-2,6-dimethyl-phenyl)pyrimidin-2-yl]sulfamoyl]benzoic acid

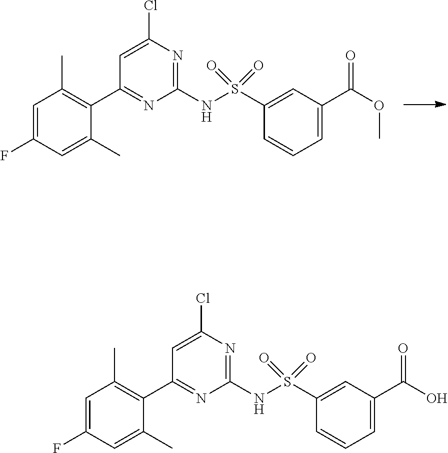 [SMILES: COC(=O)c1cccc(S(=O)(=O)Nc2nc(Cl)cc(-c3c(C)cc(F)cc3C)n2)c1 ; Cc1cc(F)cc(C)c1-c1cc(Cl)nc(NS(=O)(=O)c2cccc(C(=O)O)c2)n1]

To a solution of methyl 3-[[4-chloro-6-(4-fluoro-2,6-dimethyl-phenyl)pyrimidin-2-yl]sulfamoyl]benzoate (792 mg, 1.7605 mmol) in THE (50 mL) was added an aqueous solution of NaOH (10 mL of 1 M, 10.000 mmol) and stirred for 1 hour at room temperature. The solution was washed with diethyl ether (2×100 mL) before being acidified using 1M HCl (50 mL) and extracted with ethyl acetate (2×200 mL) before being washed with brine (200 mL). The organic layer was dried over sodium sulfate and concentrated in vacuum before being purified by prep-hplc using TFA as a buffer. The pure fractions were combined and extracted with ethyl acetate (3×150 mL) and then washed with brine (150 mL). The organic layer was then dried over sodium sulfate and concentrated in vacuum to give 3-[[4-chloro-6-(4-fluoro-2,6-dimethyl-phenyl)pyrimidin-2-yl]sulfamoyl]benzoic acid (424.4 mg, 54%). ESI-MS m/z calc. 435.0456, found 436.0 (M+1)⁺; Retention time: 2.41 minutes, LC method T. ¹H NMR (500 MHz, DMSO-d₆) δ 13.44 (s, 1H), 12.45 (s, 1H), 8.44 (t, J=1.8 Hz, 1H), 8.18 (dt, J=7.8, 1.4 Hz, 1H), 8.12 (ddd, J=7.9, 2.0, 1.2 Hz, 1H), 7.69 (t, J=7.8 Hz, 1H), 7.32 (s, 1H), 6.98 (d, J=9.7 Hz, 2H), 1.86 (s, 6H).

### Step 6: 3-[[4-(4-Fluoro-2,6-dimethyl-phenyl)-6-[(2R)-4-methyl-2-(spiro[2.3]hexan-5-ylamino)pentoxy]pyrimidin-2-yl]sulfamoyl]benzoic acid

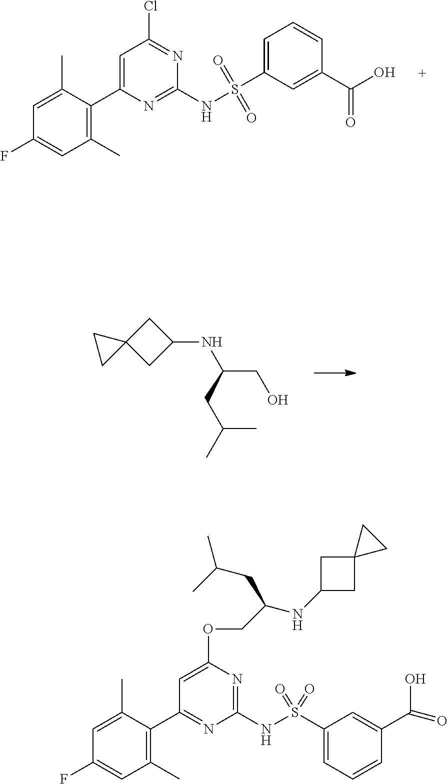 [SMILES: Cc1cc(F)cc(C)c1-c1cc(Cl)nc(NS(=O)(=O)c2cccc(C(=O)O)c2)n1 ; CC(C)C[C@H](CO)NC1CC2(CC2)C1 ; Cc1cc(F)cc(C)c1-c1cc(OC[C@@H](CC(C)C)NC2CC3(CC3)C2)nc(NS(=O)(=O)c2cccc(C(=O)O)c2)n1]

3-[[4-Chloro-6-(4-fluoro-2,6-dimethyl-phenyl)pyrimidin-2-yl]sulfamoyl]benzoic acid (76.2 mg, 0.1748 mmol), (2R)-4-methyl-2-(spiro[2.3]hexan-5-ylamino)pentan-1-ol (hydrochloride salt) (41.2 mg, 0.1762 mmol), and sodium tert-butoxide (85.2 mg, 0.8865 mmol) were combined in THE (1 mL) and stirred at room temperature for 1.5 h. The reaction mixture was partitioned between ethyl acetate and a 1M HCl solution. The organics were separated, washed with brine, dried over sodium sulfate and evaporated to give 3-[[4-(4-fluoro-2,6-dimethyl-phenyl)-6-[(2R)-4-methyl-2-(spiro[2.3]hexan-5-ylamino)pentoxy]pyrimidin-2-yl]sulfamoyl]benzoic acid (hydrochloride salt) (102 mg, 92%) ESI-MS m/z calc. 596.2469, found 597.4 (M+1)⁺; Retention time: 0.53 minutes, LC method D.

### Step 7: (11R)-6-(4-Fluoro-2,6-dimethyl-phenyl)-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (Compound 10)

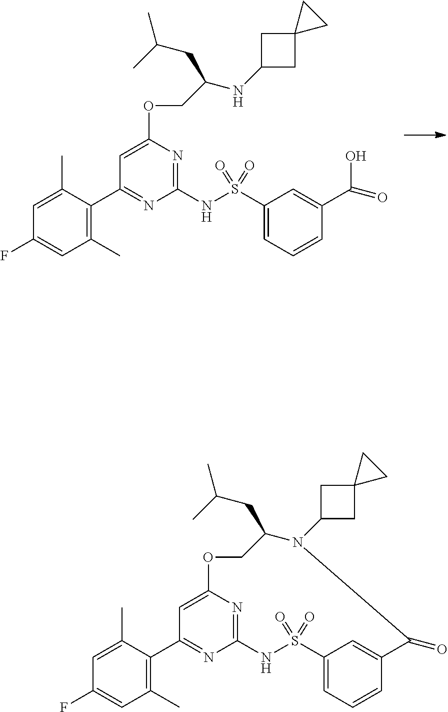 [SMILES: Cc1cc(F)cc(C)c1-c1cc(OC[C@@H](CC(C)C)NC2CC3(CC3)C2)nc(NS(=O)(=O)c2cccc(C(=O)O)c2)n1 ; Cc1cc(F)cc(C)c1-c1cc2nc(n1)NS(=O)(=O)c1cccc(c1)C(=O)N(C1CC3(CC3)C1)[C@H](CC(C)C)CO2]

3-[[4-(4-Fluoro-2,6-dimethyl-phenyl)-6-[(2R)-4-methyl-2-(spiro[2.3]hexan-5-ylamino)pentoxy]pyrimidin-2-yl]sulfamoyl]benzoic acid (hydrochloride salt) (102 mg, 0.1611 mmol), HATU (70.5 mg, 0.1854 mmol), and triethylamine (90 μL, 0.6457 mmol) were combined in DMF (3 mL) and stirred at room temperature for 4 h. The reaction mixture was filtered and purified by reverse-phase HPLC utilizing a gradient of 1-99% acetonitrile in 5 mM aqueous HCl to yield (11R)-6-(4-fluoro-2,6-dimethyl-phenyl)-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (45.1 mg, 48%) ESI-MS m/z calc. 578.2363, found 579.2 (M+1)⁺; Retention time: 2.1 minutes, LC method A.

### Example 9: Preparation of Compound 11 and Compound 12

### Step 1: tert-Butyl 2-[[(1R)-1-(hydroxymethyl)-3-methyl-butyl]amino]-7-azaspiro[3.5]nonane-7-carboxylate

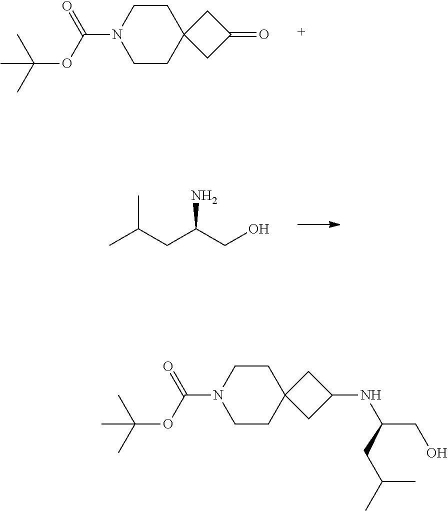

(2R)-2-amino-4-methyl-pentan-1-ol (63 mL, 522.0 mmol) was added to a solution of tert-butyl 2-oxo-7-azaspiro[3.5]nonane-7-carboxylate (100 g, 417.9 mmol) in anhydrous DCE (715 mL) under nitrogen and stirred at rt for 15 minutes. Sodium triacetoxyborohydride (266 g, 1.255 mol) was divided into 3 separate portions and added while keeping the temperature below 27° C., then stirred at rt for 18 hours. The mixture was cooled to 4° C., then HCl (420 mL of 4 M, 1.680 mol) was added very slowly, keeping the temperature between 4° C. and 12° C. (large delayed exotherm, gas evolution, and foaming in the beginning of the HCl addition). A solution of potassium carbonate (694 g, 5.022 mol) in water (650 mL) was added, while keeping the temperature below 10° C., then another portion of water (600 mL) was added, followed by MTBE (715 mL). The organic layer was separated, washed with a solution of potassium carbonate (58 g, 419.7 mmol) in water (100 mL), dried over magnesium sulfate, then concentrated to give tert-butyl 2-[[(1R)-1-(hydroxymethyl)-3-methyl-butyl]amino]-7-azaspiro[3.5]nonane-7-carboxylate (148.29 g, 100%) ESI-MS m/z calc. 340.27258, found 341.3 (M+1)⁺; Retention time: 1.15 minutes, LC method A.

### Step 2: 3-[[4-[(2R)-2-[(7-tert-Butoxycarbonyl-7-azaspiro[3.5]nonan-2-yl)amino]-4-methyl-pentoxy]-6-chloro-pyrimidin-2-yl]sulfamoyl]benzoic acid

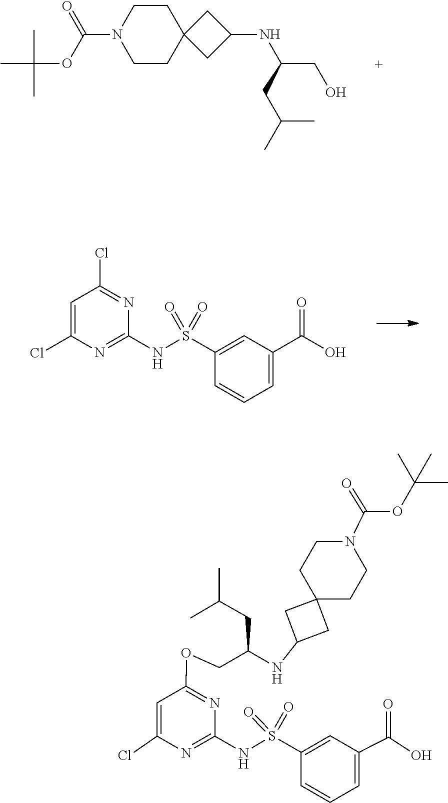 [SMILES: CC(C)C[C@H](CO)NC1CC2(CCN(C(=O)OC(C)(C)C)CC2)C1 ; O=C(O)c1cccc(S(=O)(=O)Nc2nc(Cl)cc(Cl)n2)c1 ; CC(C)C[C@H](COc1cc(Cl)nc(NS(=O)(=O)c2cccc(C(=O)O)c2)n1)NC1CC2(CCN(C(=O)OC(C)(C)C)CC2)C1]

tert-Butyl 2-[[(1R)-1-(hydroxymethyl)-3-methyl-butyl]amino]-7-azaspiro[3.5]nonane-7-carboxylate (hydrochloride salt) (1.042 g, 2.764 mmol) and 3-[(4,6-dichloropyrimidin-2-yl)sulfamoyl]benzoic acid (960.4 mg, 2.758 mmol) were combined in THE (19 mL). Sodium tert-butoxide (1.349 g, 14.04 mmol) was added and the reaction mixture was stirred at room temperature for 16 h. The reaction mixture was partitioned between ethyl acetate (40 mL) and a 1M HCl solution (40 mL). The organics were separated, washed with brine (40 mL), and dried over sodium sulfate. The reaction mixture was filtered and concentrated. The solid was further dried to give 3-[[4-[(2R)-2-[(7-tert-butoxycarbonyl-7-azaspiro[3.5]nonan-2-yl)amino]-4-methyl-pentoxy]-6-chloro-pyrimidin-2-yl]sulfamoyl]benzoic acid (1.783 g, 99%) ESI-MS m/z calc. 651.2493, found 652.3 (M+1)⁺; Retention time: 0.53 minutes, LC method D.

### Step 3: tert-Butyl 2-[(11R)-6-chloro-11-isobutyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]-7-azaspiro[3.5]nonane-7-carboxylate

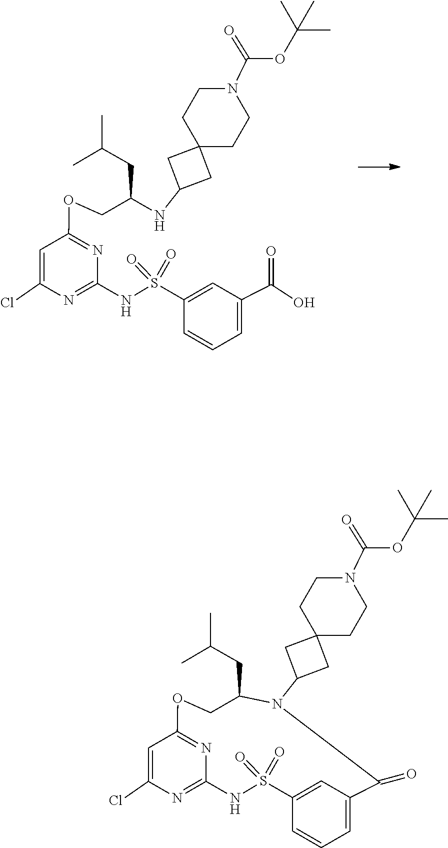 [SMILES: CC(C)C[C@H](COc1cc(Cl)nc(NS(=O)(=O)c2cccc(C(=O)O)c2)n1)NC1CC2(CCN(C(=O)OC(C)(C)C)CC2)C1 ; CC(C)C[C@@H]1COc2cc(Cl)nc(n2)NS(=O)(=O)c2cccc(c2)C(=O)N1C1CC2(CCN(C(=O)OC(C)(C)C)CC2)C1]

3-[[4-[(2R)-2-[(7-tert-butoxycarbonyl-7-azaspiro[3.5]nonan-2-yl)amino]-4-methyl-pentoxy]-6-chloro-pyrimidin-2-yl]sulfamoyl]benzoic acid (1.01 g, 1.549 mmol), HATU (591 mg, 1.554 mmol), and triethylamine (875 μL, 6.278 mmol) were combined in DMF (10 mL) and stirred at room temperature for 16 h. The reaction mixture was partitioned between ethyl acetate (20 mL) and a 1M HCl solution (20 mL). The organics were separated, washed with brine (2×20 mL), dried over sodium sulfate and evaporated. The crude material was purified by silica gel chromatography eluting with 0-70% ethyl acetate in hexanes to give tert-butyl 2-[(11R)-6-chloro-11-isobutyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]-7-azaspiro[3.5]nonane-7-carboxylate (405 mg, 41%) ESI-MS m/z calc. 633.2388, found 634.3 (M+1)⁺; Retention time: 0.8 minutes, LC method D.

### Step 4: tert-Butyl 2-[(11R)-11-isobutyl-6-(2-isopropylphenyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]-7-azaspiro[3.5]nonane-7-carboxylate (Compound 12)

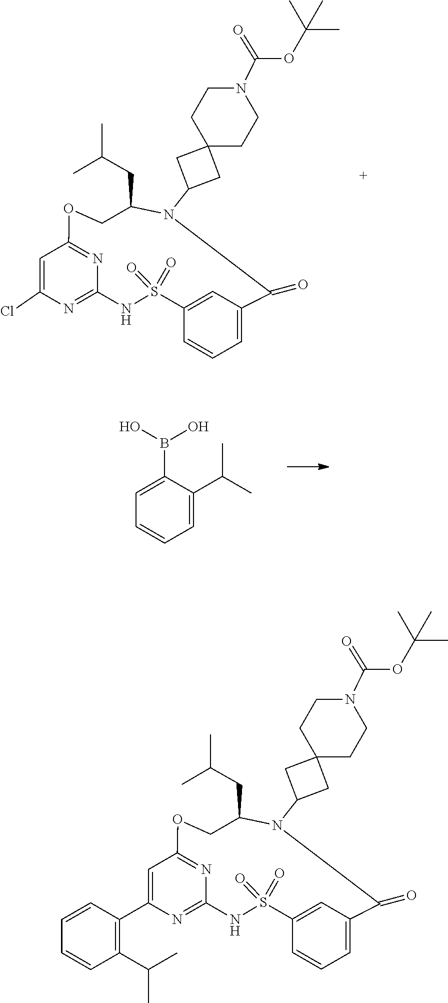 [SMILES: CC(C)C[C@@H]1COc2cc(Cl)nc(n2)NS(=O)(=O)c2cccc(c2)C(=O)N1C1CC2(CCN(C(=O)OC(C)(C)C)CC2)C1 ; CC(C)c1ccccc1B(O)O ; CC(C)C[C@@H]1COc2cc(-c3ccccc3C(C)C)nc(n2)NS(=O)(=O)c2cccc(c2)C(=O)N1C1CC2(CCN(C(=O)OC(C)(C)C)CC2)C1]

tert-Butyl 2-[(11R)-6-chloro-11-isobutyl-2,2,13-trioxo-9-oxa-2λ⁶-thia-3,5,12,19-tetrazatricyclo[12.3.1.14,8]nonadeca-1(18),4, 6, 8(19),14,16-hexaen-12-yl]-7-azaspiro[3.5]nonane-7-carboxylate (53.2 mg, 0.08389 mmol), (2-isopropylphenyl)boronic acid (28.0 mg, 0.1707 mmol), PEPPSI-Ipr (6.2 mg, 0.009111 mmol), and 2M aqueous potassium carbonate (210 μL of 2 M, 0.4200 mmol) were combined in isopropanol (530 μL) and heated at 100° C. for 16 h. The reaction mixture was filtered and purified by LC/MS utilizing a gradient of 1-99% acetonitrile in 5 mM aqueous HCl to yield tert-butyl 2-[(11R)-11-isobutyl-6-(2-isopropylphenyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]-7-azaspiro[3.5]nonane-7-carboxylate (13.4 mg, 22%) ESI-MS m/z calc. 717.356, found 718.4 (M+1)⁺; Retention time. 0.84 minutes, LC method D.

### Step 5: (11R)-12-(7-Azaspiro[3.5]nonan-2-yl)-11-isobutyl-6-(2-isopropylphenyl)-2,2-dioxo-9-oxa-2λ⁶-thia-3,5,12,19-tetrazatricyclo[12.3.1.14,8]nonadeca-1(18),4,6,8(19),14,16-hexaen-13-one

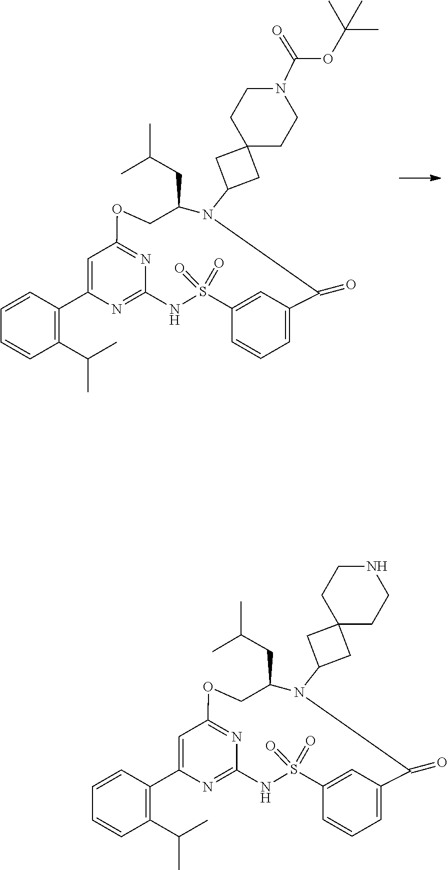 [SMILES: CC(C)C[C@@H]1COc2cc(-c3ccccc3C(C)C)nc(n2)NS(=O)(=O)c2cccc(c2)C(=O)N1C1CC2(CCN(C(=O)OC(C)(C)C)CC2)C1 ; CC(C)C[C@@H]1COc2cc(-c3ccccc3C(C)C)nc(n2)NS(=O)(=O)c2cccc(c2)C(=O)N1C1CC2(CCNCC2)C1]

tert-Butyl 2-[(11R)-11-isobutyl-6-(2-isopropylphenyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]-7-azaspiro[3.5]nonane-7-carboxylate (11 mg, 0.01532 mmol) was dissolved in 4M HCl in dioxane (0.2 mL of 4 M, 0.8000 mmol) and stirred at room temperature. After 20 min, the reaction was evaporated to give (11R)-12-(7-azaspiro[3.5]nonan-2-yl)-11-isobutyl-6-(2-isopropylphenyl)-2,2-dioxo-9-oxa-2λ⁶-thia-3,5,12,19-tetrazatricyclo[12.3.1.14,8]nonadeca-1(18),4,6,8(19),14,16-hexaen-13-one (hydrochloride salt) (10 mg, 100%) ESI-MS m/z calc. 617.3036, found 618.5 (M+1)⁺; Retention time: 0.54 minutes, LC method D.

### Step 6: (11R)-11-Isobutyl-6-(2-isopropylphenyl)-12-(7-methyl-7-azaspiro[3.5]nonan-2-yl)-2,2-dioxo-9-oxa-2 λ6-thia-3,5,12,19-tetrazatricyclo[12.3.1.14,8]nonadeca-1(18),4,6,8(19),14,16-hexaen-13-one (Compound 11)

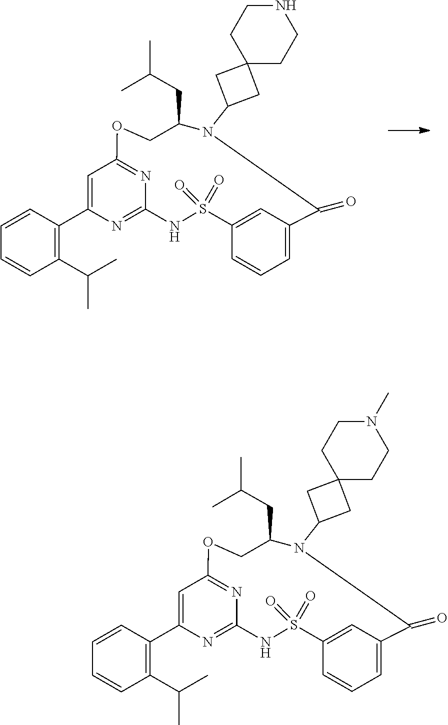 [SMILES: CC(C)C[C@@H]1COc2cc(-c3ccccc3C(C)C)nc(n2)NS(=O)(=O)c2cccc(c2)C(=O)N1C1CC2(CCNCC2)C1 ; CC(C)C[C@@H]1COc2cc(-c3ccccc3C(C)C)nc(n2)NS(=O)(=O)c2cccc(c2)C(=O)N1C1CC2(CCN(C)CC2)C1]

(11R)-1z-(-Azaspiro[3.5 ]nonan-2-yl)-11-isobutyl-6-(2-isopropylphenyl)-2,2-dioxo-9-oxa-2λ⁶-thia-3,5,12,19-tetrazatricyclo[12.3.1.14,8]nonadeca-1(18),4,6,8(19),14,16-hexaen-13-one (hydrochloride salt) (10 mg, 0.01528 mmol) was dissolved in formaldehyde (200 μL, 7.260 mmol): formic acid (200 μL) and heated at 90° C. for 20 h in a screwcap vial. The reaction was diluted with methanol (0.6 mL) and purified by LC/MS utilizing a gradient of 1-99% acetonitrile in 5 mM aqueous HCl to yield (11R)-11-isobutyl-6-(2-isopropylphenyl)-12-(7-methyl-7-azaspiro[3.5]nonan-2-yl)-2,2-dioxo-9-oxa-2λ⁶-thia-3,5,12,19-tetrazatricyclo[12.3.1.14,8]nonadeca-1(18),4,6,8(19),14,16-hexaen-13-one (hydrochloride salt) (4.2 mg, 41%) ESI-MS m/z calc. 631.3192, found 632.6 (M+1)⁺; Retention time: 1.36 minutes, LC method D.

### Example 10: Preparation of Compound 13 and Compound 14

### Step 1: tert-Butyl 2-[[(1R)-1-(hydroxymethyl)-3-methyl-butyl]amino]-7-azaspiro[3.5]nonane-7-carboxylate

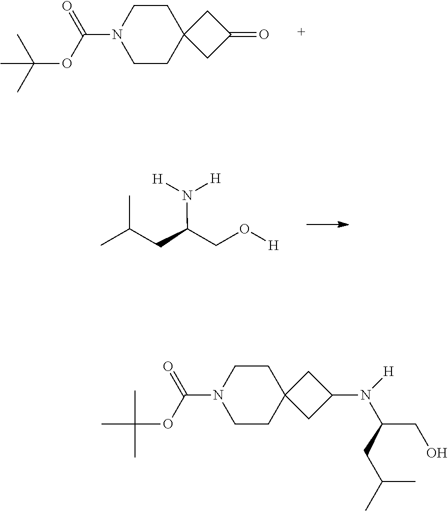

(2R)-2-Amino-4-methyl-pentan-1-ol (4.0 mL, 31.30 mmol) was added to a solution of tert-butyl 2-oxo-7-azaspiro[3.5]nonane-7-carboxylate (5.00 g, 20.89 mmol) in anhydrous DCE (30 mL) under nitrogen and stirred at rt for 30 minutes. Sodium triacetoxyborohydride (6.64 g, 31.33 mmol) was added and the reaction was stirred at rt for 1 hour 45 minutes, then another portion of sodium triacetoxyborohydride (3.33 g, 15.71 mmol) was added and the reaction was stirred for 2 hours. A third portion of sodium triacetoxyborohydride (3.33 g, 15.71 mmol) was added and the reaction mixture was stirred for 2 hours. HCl (84 mL of 1 M, 84.00 mmol) was added and stirred for 10 minutes, then a solution of potassium carbonate (12.13 g, 87.77 mmol) in water (20 mL) was added. The organic layer was separated, and the aqueous layer was extracted with DCM (30 mL). The organic layers were combined and dried over magnesium sulfate, then concentrated to give tert-butyl 2-[[(1R)-1-(hydroxymethyl)-3-methyl-butyl]amino]-7-azaspiro[3.5]nonane-7-carboxylate (8.79 g, 108%) ESI-MS m/z calc. 340.27258, found 341.3 (M+1)⁺; Retention time: 1.19 minutes; LC method A.

### Step 2: 3-[[4-[(2R)-2-[(7-tert-Butoxycarbonyl-7-azaspiro[3.5]nonan-2-yl)amino]-4-methyl-pentoxy]-6-(2,6-dimethylphenyl)pyrimidin-2-yl]sulfamoyl]benzoic acid

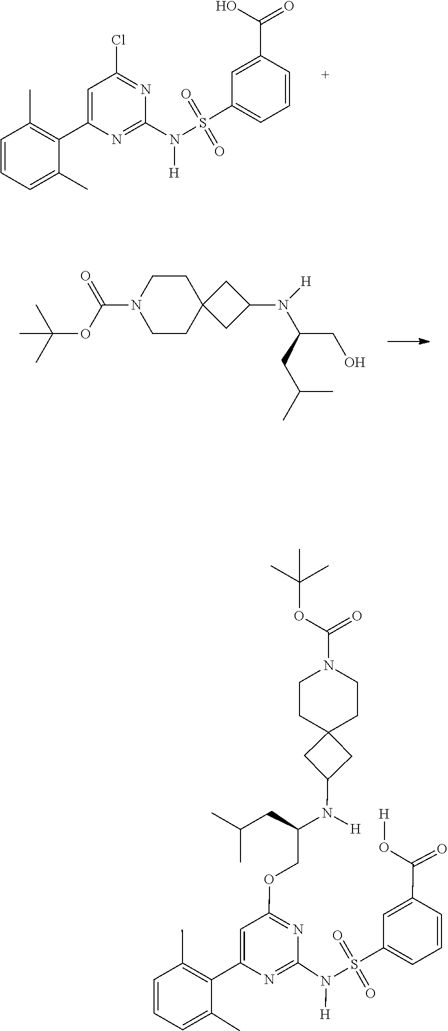 [SMILES: Cc1cccc(C)c1-c1cc(Cl)nc(NS(=O)(=O)c2cccc(C(=O)O)c2)n1 ; CC(C)C[C@H](CO)NC1CC2(CCN(C(=O)OC(C)(C)C)CC2)C1 ; Cc1cccc(C)c1-c1cc(OC[C@@H](CC(C)C)NC2CC3(CCN(C(=O)OC(C)(C)C)CC3)C2)nc(NS(=O)(=O)c2cccc(C(=O)O)c2)n1]

NaOtBu (227.1 g, 2.363 mol) was added to THE (2,000 mL) at −25° C. (exotherm). A solution of tert-butyl 2-[[(1R)-1-(hydroxymethyl)-3-methyl-butyl]amino]-7-azaspiro[3.5]nonane-7-carboxylate (178.6 g, 517.7 mmol) in THF (600 mL) was added at −15° C. 3-[[4-chloro-6-(2,6-dimethylphenyl)pyrimidin-2-yl]sulfamoyl]benzoic acid (197.5 g, 472.6 mmol) was added portion wise (delayed exotherm) in order to maintain a temperature around −15° C. It was then allowed to warm up gradually in the cold bath and stirred at rt for 14 hours. This reaction was combined with another reaction run on a similar scale using 194.5 g of 3-[[4-chloro-6-(2,6-dimethylphenyl)pyrimidin-2-yl]sulfamoyl]benzoic acid before work-up. 5400 g of ice was put into a 50 Liter reactor. Concentrated 12 M HCl (494 mL) was then added, which made this a 1 M HCl solution. EtOAc (10.4 L) was added. Then the combined reaction mixture was added under stirring. The organic layer was separated, and the aqueous layer was set aside. The organic layer was washed with brine (2600 mL), then the brine layer was separated and added to the first aqueous layer. The combined aqueous layer was extracted with EtOAc (1500 mL), then the organic layers were combined and dried over magnesium sulfate. The mixture was then concentrated in a 20 Liter flask under reduced pressure in a water bath at 45° C. The resulting product was combined with another batch (starting 6.4 g of the chloro pyrimidine starting material) and the combined solids were recrystallized in EtOAc to give 503.4 g of 3-[[4-[(2R)-2-[(7-tert-butoxycarbonyl-7-azaspiro[3.5]nonan-2-yl)amino]-4-methyl-pentoxy]-6-(2,6-dimethylphenyl)pyrimidin-2-yl]sulfamoyl]benzoic acid (hydrochloride salt) (503.4 g, 68% adjusted yield). ESI-MS m/z calc. 721.3509, found 722.2 (M+1)⁺; Retention time: 1.45 minutes; LC method A.

### Step 3: tert-Butyl 2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (Compound 13)

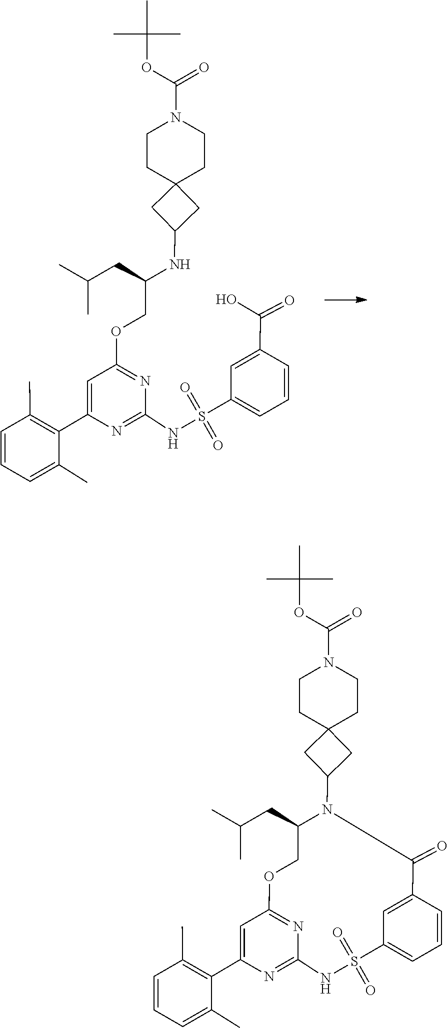 [SMILES: Cc1cccc(C)c1-c1cc(OC[C@@H](CC(C)C)NC2CC3(CCN(C(=O)OC(C)(C)C)CC3)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CCN(C(=O)OC(C)(C)C)CC3)C1)[C@H](CC(C)C)CO2]

3-[[4-[(2R)-2-[(7-tert-butoxycarbonyl-7-azaspiro[3.5]nonan-2-yl)amino]-4-methyl-pentoxy]-6-(2,6-dimethylphenyl)pyrimidin-2-yl]sulfamoyl]benzoic acid (hydrochloride salt)(52 g, 68.57 mmol) was dissolved in DMF (1 L), treated first with DIEA (48 mL, 275.6 mmol) and immediately followed by HATU (39 g, 102.6 mmol). The solution was stirred at room temperature for 13 hours. The deep orange solution was evaporated under reduced pressure at 45-50° C. to an orange mass and treated with citric acid (550 mL of 1 M, 550.0 mmol) to give a light brown suspension which was stirred at room temperature for 2 h. The solid was collected by filtration and the wet solid was dissolved in DCM, which was washed with 1M citric acid and brine and the aqueous phases were back extracted once with DCM. The combined organic phases were dried (magnesium sulfate), treated with charcoal, filtered over Celite and evaporated to give 52.3 g of a deep orange foam. Half of the crude product (26 g) was purified by chromatography over silica gel (330 g, liquid load with DCM) with a linear gradient of hexane to 50% ethyl acetate to give tert-Butyl 2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (18.86 g, 78%) as a yellow foam. ESI-MS m/z calc. 703.34033, found 704.0 (M+1)⁺; Retention time: 2.19 minutes; LC method A.

### Step 4: (11R)-12-(7-Azaspiro[3.5]nonan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 14)

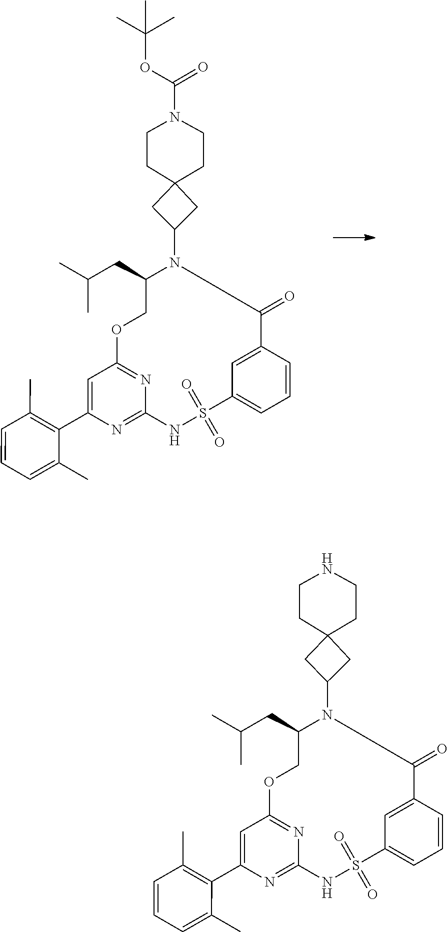 [SMILES: Cc1cccc(C)c1-c1cc2nc(n1)NS(=O)(=O)c1cccc(c1)C(=O)N(C1CC3(CCN(C(=O)OC(C)(C)C)CC3)C1)[C@H](CC(C)C)CO2 ; Cc1cccc(C)c1-c1cc2nc(n1)NS(=O)(=O)c1cccc(c1)C(=O)N(C1CC3(CCNCC3)C1)[C@H](CC(C)C)CO2]

To tert-butyl 2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (62 g, 88.08 mmol) in MeOH (300 mL) and Toluene (150 mL) was added HCl (70 mL of 4 M, 280.0 mmol). The mixture was stirred at ambient temperature for 4 h. The solvents were removed in vacuo. The semi solid was evaporated twice from MeTHF (300 mL) and the solid stirred at ambient temperature in MeTHF (300 mL) for 48 h. The precipitate was collected using a M frit and washed with MeTHF. The solid was air dried for 4 h, then in vacuo at 45° C. for 24 h to give (11R)-12-(7-azaspiro[3.5]nonan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt)(58.24 g, 103%). ¹H NMR (500 MHz, DMSO-d₆) δ 8.56 (d, J=25.7 Hz, 2H), 8.39 (s, 1H), 7.91 (d, J=7.1 Hz, 1H), 7.67 (d, J=8.1 Hz, 2H), 7.26 (t, J=7.6 Hz, 1H), 7.12 (d, J=7.7 Hz, 2H), 6.39 (s, 1H), 5.10 (dd, J=10.6, 4.3 Hz, 1H), 4.36 (t, J=11.0 Hz, 2H), 4.20 (s, 1H), 4.06 (p, J=8.9 Hz, 2H), 3.79-3.67 (m, 2H), 3.01 (d, J=33.4 Hz, 4H), 2.83 (q, J=10.8 Hz, 2H), 2.11-1.81 (m, 12H), 1.63 (t, J=12.6 Hz, 1H), 1.38-1.24 (m, 1H), 1.12 (d, J=6.1 Hz, 2H), 0.74 (d, J=6.7 Hz, 3H). ESI-MS m/z calc. 603.2879, found 604.2 (M+1)⁺; Retention time: 1.79 minutes; LC method I.

### Example 11: Preparation of Compound 15

### Step 1: (11R)-6-(2,6-Dimethylphenyl)-11-isobutyl-12-(7-methyl-7-azaspiro[3.5]nonan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 15)

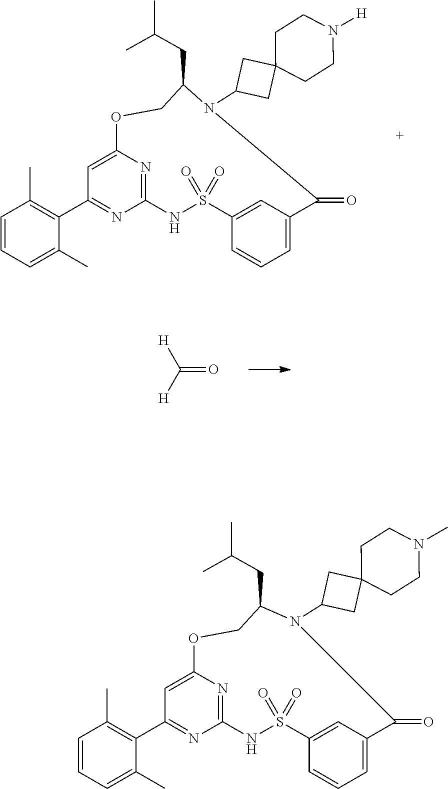 [SMILES: Cc1cccc(C)c1-c1cc2nc(n1)NS(=O)(=O)c1cccc(c1)C(=O)N(C1CC3(CCNCC3)C1)[C@H](CC(C)C)CO2 ; C=O ; Cc1cccc(C)c1-c1cc2nc(n1)NS(=O)(=O)c1cccc(c1)C(=O)N(C1CC3(CCN(C)CC3)C1)[C@H](CC(C)C)CO2]

(11R)-12-(7-azaspiro[3.5]nonan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (110 mg, 0.1718 mmol) was dissolved in formic acid (1 mL) and combined with aqueous formaldehyde (1.2 mL, 43.56 mmol) and heated to 90° C. for 20 hours in a screwcap vial. The reaction mixture was then partially concentrated under reduced pressure, diluted with methanol, filtered, then purified by reverse phase HPLC (1-70% ACN in water, HCl modifier, 15 minutes) in two batches. The fractions containing product were combined and concentrated to give after drying as a white powder, (11R)-6-(2,6-dimethylphenyl)-11-isobutyl-12-(7-methyl-7-azaspiro[3.5]nonan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (39 mg, 34%), ESI-MS m/z calc. 617.3036, found 618.6 (M+1)⁺; Retention time: 1.24 minutes; LC method A.; ¹H NMR (400 MHz, DMSO) δ 9.68 (s, 1H), 8.39 (d, J=2.5 Hz, 1H), 7.91 (d, J=6.9 Hz, 1H), 7.68 (s, 2H), 7.26 (t, J=7.5 Hz, 1H), 7.13 (d, J=7.7 Hz, 2H), 6.40 (s, 1H), 5.10 (d, J=10.5 Hz, 1H), 4.35 (td, J=10.9, 7.1 Hz, 1H), 4.14-4.00 (m, 1H), 3.73 (s, 1H), 3.39-3.25 (m, 2H), 2.89 (tt, J=24.2, 12.6 Hz, 3H), 2.79-2.70 (m, 3H), 2.06 (ddd, J=35.5, 23.2, 9.2 Hz, 9H), 1.84-1.69 (m, 2H), 1.61 (t, J=11.9 Hz, 1H), 1.27 (dd, J=17.4, 6.5 Hz, 2H), 1.19-1.08 (m, 1H), 0.91-0.82 (m, 1H), 0.74 (dd, J=11.6, 6.6 Hz, 3H), 0.20 (t, J=5.7 Hz, 3H).

### Example 12: Preparation of Compound 16

### Step 1: (11R)-12-(7-Acetyl-7-azaspiro[3.5]nonan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 16)

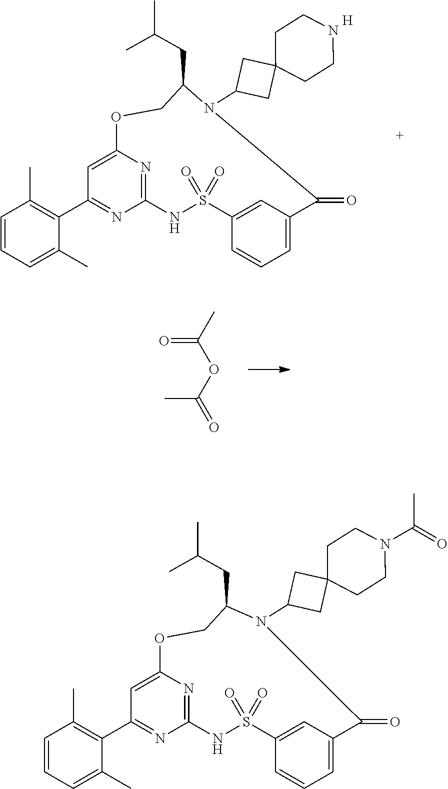

(11R)-12-(7-azaspiro[3.5]nonan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (100 mg, 0.1562 mmol) was combined in dichloromethane (1 mL) with acetic anhydride (40 μL, 0.4239 mmol), and triethylamine (110 μL, 0.7892 mmol). The reaction mixture was stirred at room temperature for 15 minutes then diluted with ethyl acetate and washed with aqueous 1M HCl. The aqueous layer was extracted two additional times with ethyl acetate, and the combined organics were washed with brine then dried over sodium sulfate and concentrated. The resulting crude material was purified by silica gel chromatography using a gradient of 0-10% methanol in dichloromethane (elutes around 5% methanol). The fractions containing product were combined and concentrated to give as a white powder, (11R)-12-(7-acetyl-7-azaspiro[3.5]nonan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (32 mg, 32%)¹H NMR (400 MHz, DMSO) δ 13.28-12.53 (m, 1H), 8.38 (s, 1H), 7.90 (s, 1H), 7.68 (s, 2H), 7.25 (d, J=8.1 Hz, 1H), 7.12 (d, J=7.5 Hz, 2H), 6.38 (s, 1H), 5.23-4.99 (m, 1H), 4.37 (t, J=10.9 Hz, 1H), 4.13-4.00 (m, 1H), 3.72 (s, 1H), 3.40 (d, J=20.7 Hz, 2H), 2.82 (dt, J=19.1, 9.3 Hz, 2H), 2.15-2.03 (m, 3H), 2.02-1.87 (m, 7H), 1.69 (dd, J=11.7, 5.5 Hz, 2H), 1.60 (t, J=8.2 Hz, 3H), 1.33-1.22 (m, 2H), 1.17-1.09 (m, 1H), 0.91-0.77 (m, 2H), 0.73 (d, J=6.6 Hz, 3H), 0.20 (d, J=6.0 Hz, 3H). ESI-MS m/z calc. 645.29846, found 646.5 (M+1)⁺; Retention time: 1.66 minutes; LC method A.

### Example 13: Preparation of Compound 17

### Step 1: (11R)-6-(2,6-Dimethylphenyl)-11-isobutyl-12-(7-isopropyl-7-azaspiro[3.5]nonan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 17)

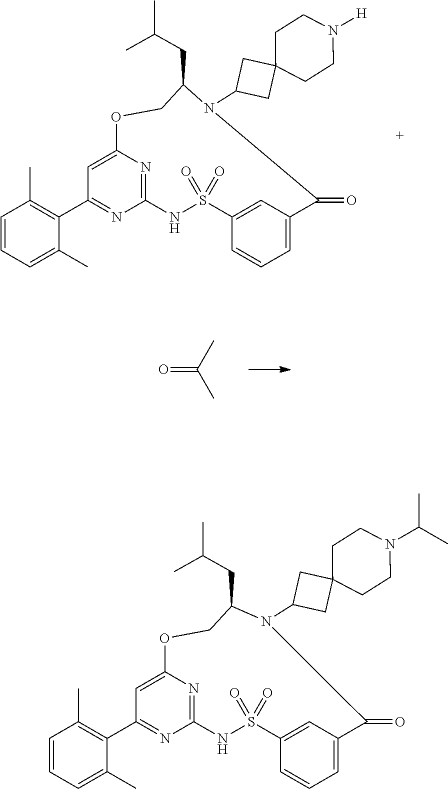

(11R)-12-(7-Azaspiro[3.5]nonan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (100 mg, 0.1562 mmol) was combined with acetone (500 μL, 6.810 mmol) in dichloroethane (500 μL) and stirred at room temperature. After 5 minutes, sodium triacetoxyborohydride (150 mg, 0.7077 mmol) was added and the reaction temperature was increased to 50° C. After 90 minutes, an additional portion of sodium triacetoxyborohydride (150 mg, 0.7077 mmol), was added and stirring was continued for an additional 90 minutes at 50° C. The reaction mixture was then concentrated, diluted with 1.5 mL 1:1 DMSO/methanol and 0.2 mL acetic acid, filtered, then purified by reverse phase HPLC (1-99% ACN in water, HCl modifier, 15 min run) to give as a white solid, (11R)-6-(2,6-dimethylphenyl)-11-isobutyl-12-(7-isopropyl-7-azaspiro[3.5]nonan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (38 mg, 35%) ESI-MS m/z calc. 645.3349, found 646.6 (M+1)⁺; Retention time: 1.32 minutes (LC method A). ¹H NMR (400 MHz, DMSO) δ 9.86 (d, J=28.1 Hz, 1H), 8.39 (d, J=6.3 Hz, 1H), 7.98-7.83 (m, 1H), 7.68 (dd, J=7.5, 4.7 Hz, 2H), 7.26 (t, J=7.6 Hz, 1H), 7.13 (d, J=7.5 Hz, 2H), 6.40 (s, 1H), 5.10 (dt, J=10.1, 3.8 Hz, 1H), 4.37 (t, J=11.0 Hz, 1H), 4.09 (h, J=9.2, 8.3 Hz, 1H), 3.71 (dd, J=7.4, 3.5 Hz, 1H), 3.41 (d, J=5.1 Hz, 1H), 3.37-3.21 (m, 3H), 2.96-2.70 (m, 4H), 2.24-2.05 (m, 5H), 2.04-1.85 (m, 7H), 1.63 (t, J=12.3 Hz, 1H), 1.28 (d, J=6.4 Hz, 7H), 1.21-1.06 (m, 1H), 0.74 (t, J=6.3 Hz, 3H), 0.20 (d, J=6.2 Hz, 3H).

### Example 14: Preparation of Compound 18

### Step 1: 7-(2,2,2-Trifluoroethyl)-7-azaspiro[3.5]nonan-2-one

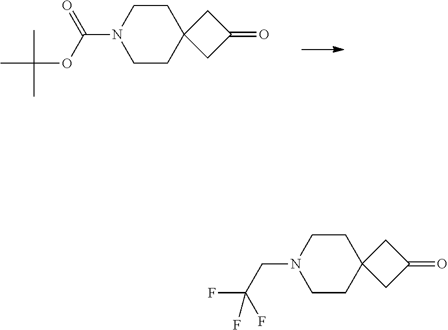 [SMILES: CC(C)(C)OC(=O)N1CCC2(CC1)CC(=O)C2 ; O=C1CC2(CCN(CC(F)(F)F)CC2)C1]

tert-Butyl 2-oxo-7-azaspiro[3.5]nonane-7-carboxylate (250 mg, 1.045 mmol) was combined in dichloromethane (2.5 mL) with HCl (2.5 mL of 4 M, 10.00 mmol) and stirred for 30 minutes at room temperature. The reaction mixture was then concentrated to give a slightly yellow amorphous solid. This material was triturated in diethyl ether then collected by filtration as an off white solid, 7-azaspiro[3.5]nonan-2-one (hydrochloride salt)(180 mg, 98%) ESI-MS m/z calc. 139.09972, found 140.0 (M+1)⁺; Retention time: 0.09 minutes (LC method D).

The product was dissolved in a screwcap vial with anhydrous acetone (4 mL), 2,2,2-trifluoroethyl trifluoromethanesulfonate (188 μL, 1.305 mmol) and triethylamine (750 μL, 5.381 mmol) and heated to 55° C. for 5 hours. The reaction mixture was then cooled to room temperature and concentrated. The resulting residue was dissolved in 15 mL dichloromethane and washed with 15 mL aqueous sodium bicarbonate. The aqueous layer was extracted with an additional 2×15 mL dichloromethane and the combined organics were washed with brine, dried over sodium sulfate and concentrated to give a reddish oil that was used in the next step without further purification 7-(2,2,2-trifluoroethyl)-7-azaspiro[3.5]nonan-2-one (215 mg, 93%). ESI-MS m/z calc. 221.10275, found 222.1 (M+1)⁺; Retention time: 0.21 minutes (LC method D).

### Step 2: (11R)₆-(2,6-Dimethylphenyl)-11-isobutyl-2,2-dioxo-12-[7-(2,2,2-trifluoroethyl)-7-azaspiro[3.5]nonan-2-yl]-9-oxa-2λ⁶-thia-3,5,12,19-tetrazatricyclo[12.3.1.14,8]nonadeca-1(18),4(19),5,7,14,16-hexaen-13-one (Compound 18)

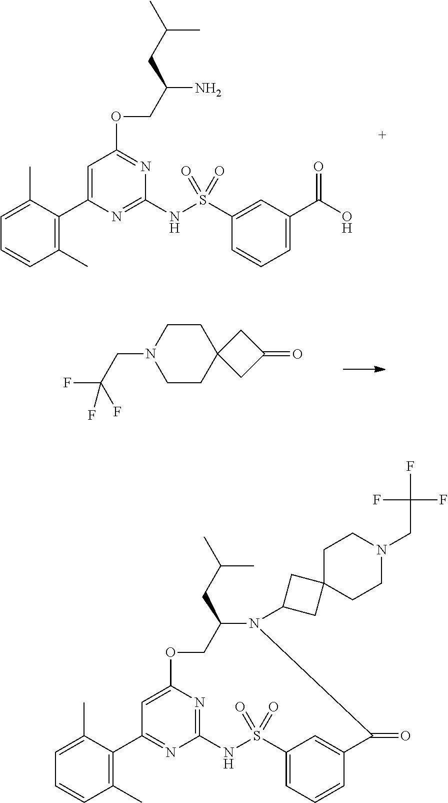 [SMILES: Cc1cccc(C)c1-c1cc(OC[C@H](N)CC(C)C)nc(NS(=O)(=O)c2cccc(C(=O)O)c2)n1 ; O=C1CC2(CCN(CC(F)(F)F)CC2)C1 ; Cc1cccc(C)c1-c1cc2nc(n1)NS(=O)(=O)c1cccc(c1)C(=O)N(C1CC3(CCN(CC(F)(F)F)CC3)C1)[C@H](CC(C)C)CO2]

3-[[4-[(2R)-2-Amino-4-methyl-pentoxy]-6-(2,6-dimethylphenyl)pyrimidin-2-yl]sulfamoyl]benzoic acid (hydrochloride salt) (50 mg, 0.09345 mmol) and 7-(2,2,2-trifluoroethyl)-7-azaspiro[3.5]nonan-2-one (approximately 41.35 mg, 0.1869 mmol) were combined in DCM (0.5 mL) and sodium triacetoxyborohydride (approximately 39.61 mg, 0.1869 mmol) was added. After one hour, an additional portion of sodium triacetoxyborohydride (approximately 39.61 mg, 0.1869 mmol) was added followed by an additional portion of 7-(2,2,2-trifluoroethyl)-7-azaspiro[3.5]nonan-2-one (approximately 41.35 mg, 0.1869 mmol). After an additional five hours, the reaction mixture was poured into a separatory funnel containing 20 mL 0.5 M HCl and 20 mL ethyl acetate. The layers were separated, and the aqueous was extracted three additional times with 15 mL ethyl acetate. 20 mL brine was added to the aqueous layer and it was further extracted 10×15 mL ethyl acetate. The combined organics were dried over sodium sulfate and concentrated. The resulting crude was combined in DMF (3.5 mL) with HATU (approximately 53.31 mg, 0.1402 mmol), and DIPEA (approximately 72.47 mg, 97.67 μL, 0.5607 mmol) was added. After stirring at room temperature for two hours, the reaction mixture was filtered and purified by reverse phase HPLC (1-70% ACN, HCl modifier, 15 minutes) in two batches to give the corresponding (11R)-6-(2,6-dimethylphenyl)-11-isobutyl-2,2-dioxo-12-[7-(2,2,2-trifluoroethyl)-7-azaspiro[3.5]nonan-2-yl]-9-oxa-2λ⁶-thia-3,5,12,19-tetrazatricyclo[12.3.1.14,8]nonadeca-1(18),4(19),5,7,14,16-hexaen-13-one (hydrochloride salt) (12 mg, 17%). ESI-MS m/z calc. 685.29095, found 686.5 (M+1)⁺; Retention time: 1.5 minutes; LC method A.

### Example 15: Preparation of Compound 19

### Step 1: Methyl 2-[(11R)-6-(2,6-dimethylphenyl)-11-isobutyl-2,2,13-trioxo-9-oxa-2%⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (Compound 19)

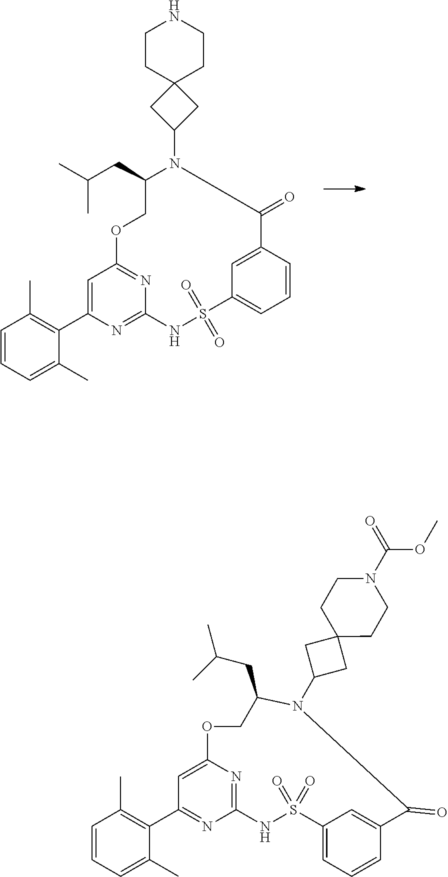 [SMILES: Cc1cccc(C)c1-c1cc2nc(n1)NS(=O)(=O)c1cccc(c1)C(=O)N(C1CC3(CCNCC3)C1)[C@H](CC(C)C)CO2 ; COC(=O)N1CCC2(CC1)CC(N1C(=O)c3cccc(c3)S(=O)(=O)Nc3nc(cc(-c4c(C)cccc4C)n3)OC[C@H]1CC(C)C)C2]

Methylchloroformate (224 mL of 0.25 M, 56.00 mmol) (0.25 M in DCM) was added to a solution of (11R)-12-(7-azaspiro[3.5]nonan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (37.3 g, 56.51 mmol) and triethylamine (26.0 mL, 186.5 mmol) in DCM (1000 mL) under nitrogen while maintaining a temperature between −18° C. and −10° C., and the mixture was stirred at −10° C. for 35 minutes. A solution of citric acid (43.5 g, 226.4 mmol) in water (200 mL) was added, stirred for 45 minutes, then the organic layer was separated, dried over magnesium sulfate, and concentrated. The product was purified by running two successive silica columns, using a gradient eluent of 0% to 3% MeOH in DCM to obtain methyl 2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (26.52 g, 71%). ESI-MS m/z calc. 661.2934, found 662.2 (M+1)⁺; Retention time: 1.92 minutes; LC method A. ¹H NMR (499 MHz, dimethylsulfoxide-d₆) δ 13.57-11.47 (bs, 1H), 8.38 (s, 1H), 7.90 (d, J=7.2 Hz, 1H), 7.77-7.55 (m, 2H), 7.25 (t, J=7.5 Hz, 1H), 7.12 (d, J=7.0 Hz, 2H), 6.37 (s, 1H), 5.09 (dd, J=10.7, 4.3 Hz, 1H), 4.36 (t, J=11.1 Hz, 1H), 4.05 (p, J=8.9 Hz, 1H), 3.72 (td, J=11.0, 10.5, 5.4 Hz, 1H), 3.58 (s, 3H), 3.36 (t, J=5.5 Hz, 2H), 3.29 (t, J=5.6 Hz, 2H), 2.80 (dt, J=16.2, 9.7 Hz, 2H), 2.09-1.93 (m, 8H), 1.70-1.56 (m, 5H), 1.38-1.24 (m, 1H), 1.20-1.06 (m, 1H), 0.73 (d, J=6.6 Hz, 3H), 0.20 (d, J=6.3 Hz, 3H).

### Example 16: Preparation of Compound 20

### Step 1: (11R)-6-(2,6-Dimethylphenyl)-11-isobutyl-2,2-dioxo-12-[7-(3,3,3-trifluoropropyl)-7-azaspiro[3.5]nonan-2-yl]-9-oxa-2λ⁶-thia-3,5,12,19-tetrazatricyclo[12.3.1.14,8]nonadeca-1(18),4(19),5,7,14,16-hexaen-13-one (Compound 20)

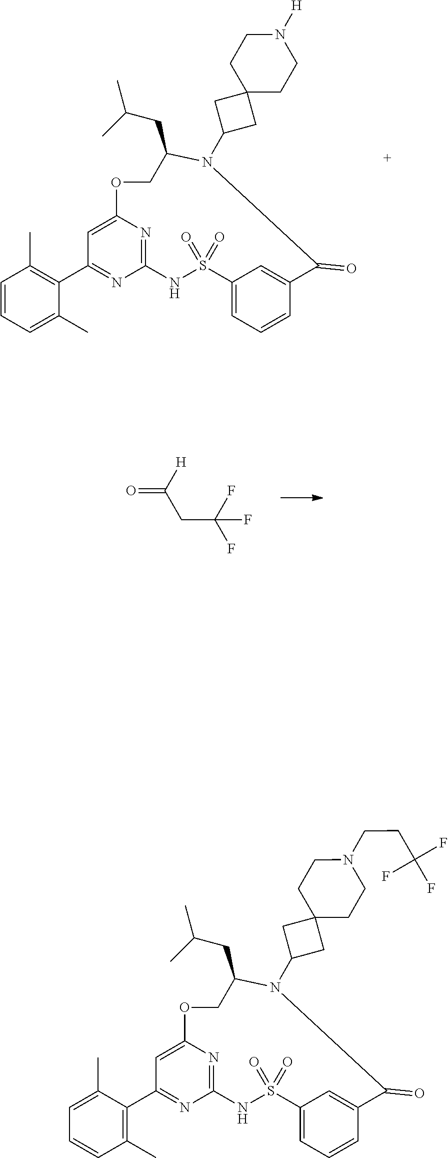 [SMILES: Cc1cccc(C)c1-c1cc2nc(n1)NS(=O)(=O)c1cccc(c1)C(=O)N(C1CC3(CCNCC3)C1)[C@H](CC(C)C)CO2 ; O=CCC(F)(F)F ; Cc1cccc(C)c1-c1cc2nc(n1)NS(=O)(=O)c1cccc(c1)C(=O)N(C1CC3(CCN(CCC(F)(F)F)CC3)C1)[C@H](CC(C)C)CO2]

(11R)-12-(7-Azaspiro[3.5]nonan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (20 mg, 0.03124 mmol) was combined with 3,3,3-trifluoropropanal (7 mg, 0.06247 mmol) in DCM (0.5 mL) and sodium triacetoxyborohydride (26 mg, 0.1227 mmol) was added. The reaction was stirred at room temperature for 1 hour, then was partially concentrated, diluted with 1:1 DMSO and methanol, filtered and purified by reverse phase HPLC (1-99% ACN in water, HCl modifier) to give (11R)-6-(2,6-dimethylphenyl)-11-isobutyl-2,2-dioxo-12-[7-(3,3,3-trifluoropropyl)-7-azaspiro[3.5]nonan-2-yl]-9-oxa-2λ⁶-thia-3,5,12,19-tetrazatricyclo[12.3.1.14,8]nonadeca-1(18),4(19),5,7,14,16-hexaen-13-one (hydrochloride salt). ESI-MS m/z calc. 699.30664, found 700.6 (M+1)⁺; Retention time: 1.36 minutes; LC method A.

### Example 17: Preparation of Compound 21

### Step 1: Isopropyl 2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (Compound 21)

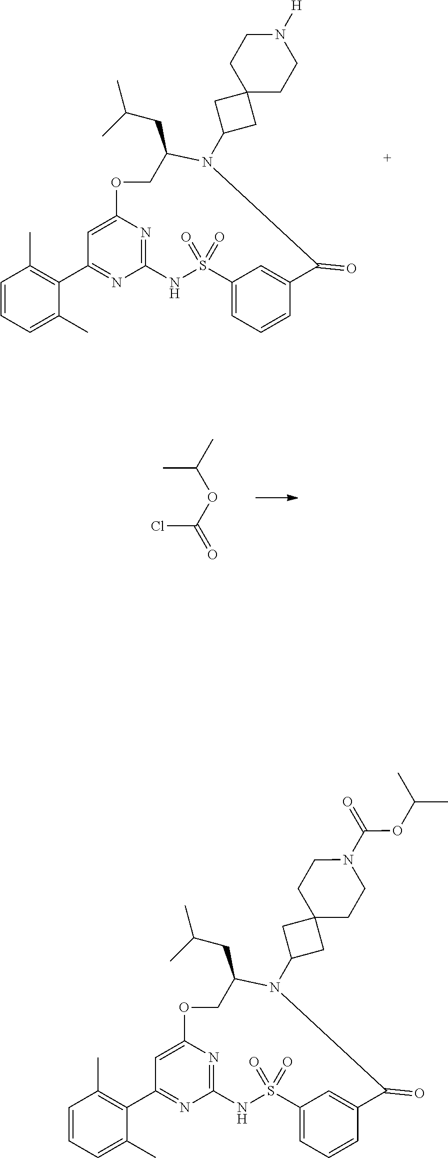

(11R)-12-(7-Azaspiro[3.5]nonan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (52.5 mg, 0.08200 mmol), isopropyl chloroformate (85 μL of 2 M, 0.1700 mmol), and triethylamine (48 μL, 0.3444 mmol) were combined in DMF (1 mL) and stirred at room temperature for 15 min. The reaction mixture was filtered and purified by LC/MS utilizing a gradient of 1-99% acetonitrile in 5 mM aqueous HCl to yield isopropyl 2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (32.1 mg, 57%) ESI-MS m/z calc. 689.3247, found 690.3 (M+1)⁺; Retention time: 2.11 minutes (LC method A).

### Example 18: Preparation of Compound 22

### Step 1: (11R)-6-(2,6-dimethylphenyl)-11-isobutyl-12-[7-(2-methoxyethyl)-7-azaspiro[3.5]nonan-2-yl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 22)

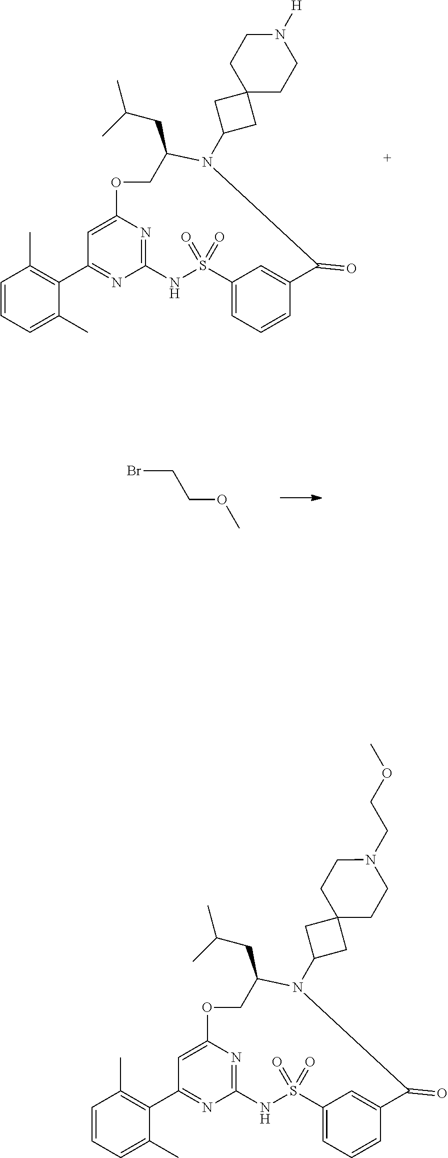

(11R)-12-(7-Azaspiro[3.5]nonan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (25 mg, 0.03905 mmol) was combined with triethylamine (35 μL, 0.2511 mmol) in acetonitrile (0.5 mL) in a screwcap vial, and 1-bromo-2-methoxy-ethane (4.5 μL, 0.04788 mmol) was added. The reaction mixture was heated to 55° C. for 20 hours. The reaction mixture was cooled to room temperature, diluted with methanol, filtered, then purified by reverse phase HPLC (15-75ACN in water, HCl modifier, 15 min run). One of the main fractions overlapped with starting material and was re-purified by reverse phase HPLC (1-50% ACN in water, HCl modifier). The pure fractions from both runs were combined and dried to give (11R)-6-(2,6-dimethylphenyl)-11-isobutyl-12-[7-(2-methoxyethyl)-7-azaspiro[3.5]nonan-2-yl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (8.3 mg, 30%); ESI-MS m/z calc. 661.3298, found 662.7 (M+1)⁺; Retention time: 1.38 minutes; LC method A.

### Example 19: Preparation of Compound 23

### Step 1: (11R)-6-(2,6-dimethylphenyl)-11-isobutyl-12-[7-(3-methoxypropyl)-7-azaspiro[3.5]nonan-2-yl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 23)

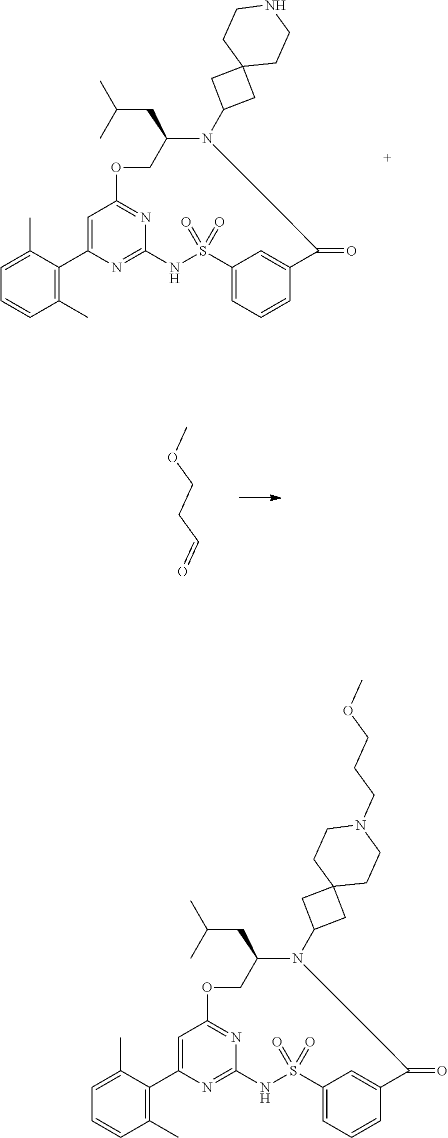

(11R)-12-(7-Azaspiro[3.5]nonan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (40 mg, 0.06248 mmol) was combined with 3-methoxypropanal (33 mg, 0.3746 mmol) in DCM (0.3 mL) and stirred for 15 minutes at room temperature. Then, sodium triacetoxyborohydride (100 mg, 0.4718 mmol) was added and the reaction mixture was stirred for an additional 15 minutes. The reaction mixture was then quenched with several drops of 1M HCl, diluted slightly with methanol and stirred for 5 minutes. The reaction was partially concentrated, diluted with 1:1 methanol/DMSO, filtered, and purified by reverse phase HPLC 1-70% ACN in water, HCl modifier, to give (11R)-6-(2,6-dimethylphenyl)-11-isobutyl-12-[7-(3-methoxypropyl)-7-azaspiro[3.5]nonan-2-yl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (29.4 mg, 65%). ¹H NMR (400 MHz, DMSO-d₆) δ 13.08 (s, 1H), 9.64 (s, 1H), 8.39 (s, 1H), 7.91 (s, 1H), 7.68 (s, 2H), 7.26 (t, J=7.6 Hz, 1H), 7.12 (d, J=7.6 Hz, 2H), 6.39 (s, 1H), 5.10 (d, J=10.4 Hz, 1H), 4.35 (td, J=11.2, 6.0 Hz, 1H), 4.18-4.03 (m, 1H), 3.73 (s, 1H), 3.42 (s, 1H), 3.41 (s, 3H), 3.25 (s, 3H), 3.07 (s, 2H), 2.97-2.73 (m, 4H), 2.23-1.71 (m, 13H), 1.63 (q, J=11.4, 10.9 Hz, 1H), 1.29 (s, 1H), 1.14 (dd, J=15.1, 9.1 Hz, 1H), 0.74 (dd, J=10.4, 6.6 Hz, 3H), 0.20 (t, J=5.3 Hz, 3H). ESI-MS m/z calc. 675.34546, found 676.7 (M+1)⁺; Retention time: 1.32 minutes; LC method A.

### Example 20: Preparation of Compound 24

### Step 1: (11R)-6-(2,6-Dimethylphenyl)-11-isobutyl-12-[7-(oxetan-3-yl)-7-azaspiro[3.5]nonan-2-yl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 24)

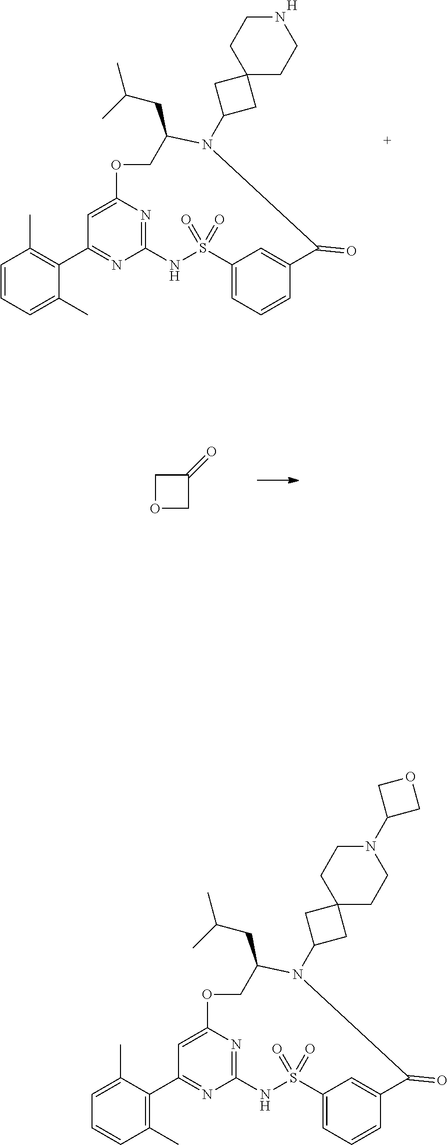

(11R)-12-(7-Azaspiro[3.5]nonan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (20 mg, 0.03124 mmol) and the oxetan-3-one (10 mg, 0.1388 mmol) were combined in DCM (0.3 mL) and sodium triacetoxyborohydride (approximately 39.72 mg, 0.1874 mmol) was added. The reaction was stirred for one hour at room temperature, then was partially concentrated, dissolved in 1:1 methanol/DMSO, filtered, and purified by reverse phase HPLC (1-70% ACN in water HCl modifier, 15 min run) to give the indicated (11R)-6-(2,6-dimethylphenyl)-11-isobutyl-12-[7-(oxetan-3-yl)-7-azaspiro[3.5]nonan-2-yl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (5.9 mg, 27%). ESI-MS m/z calc. 659.31415, found 660.7 (M+1)⁺; Retention time: 1.27 minutes; LC method A.

### Example 21: Preparation of Compound 25

### Step 1: (11R)-12-{7-[2-(Benzyloxy)acetyl]-7-azaspiro[3.5]nonan-2-yl}-6-(2,6-dimethylphenyl)-11-(2-methylpropyl)-9-oxa-2λ⁶-thia-3,5,12,19-tetraazatricyclo[12.3.1.14,8]nonadeca-1(17),4(19),5,7,14(18),15-hexaene-2,2,13-trione

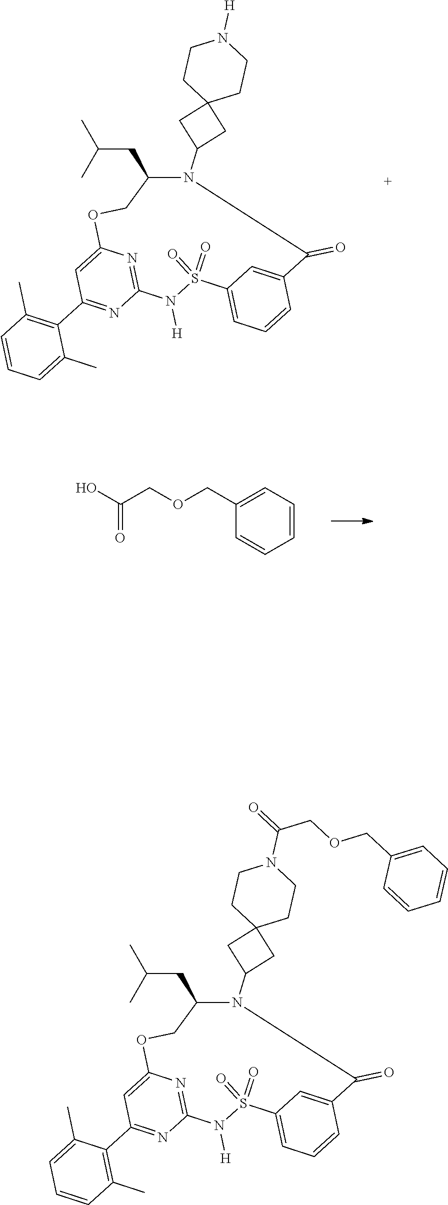 [SMILES: Cc1cccc(C)c1-c1cc2nc(n1)NS(=O)(=O)c1cccc(c1)C(=O)N(C1CC3(CCNCC3)C1)[C@H](CC(C)C)CO2 ; O=C(O)COCc1ccccc1 ; Cc1cccc(C)c1-c1cc2nc(n1)NS(=O)(=O)c1cccc(c1)C(=O)N(C1CC3(CCN(C(=O)COCc4ccccc4)CC3)C1)[C@H](CC(C)C)CO2]

(11R)-12-(7-Azaspiro[3.5]nonan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (25 mg, 0.03905 mmol), 2-benzyloxyacetic acid (approximately 6.489 mg, 5.584 μL, 0.03905 mmol), HATU (approximately 14.85 mg, 0.03905 mmol), and triethylamine (approximately 15.81 mg, 21.78 μL, 0.1562 mmol) were combined in DMF (1 mL) and stirred at room temperature for 15 min. The reaction mixture was filtered and purified by LC/MS utilizing a gradient of 1-99% acetonitrile in 5 mM aqueous HCl to yield (11R)-12-{7-[2-(benzyloxy)acetyl]-7-azaspiro[3.5]nonan-2-yl}-6-(2,6-dimethylphenyl)-11-(2-methylpropyl)-9-oxa-2λ⁶-thia-3,5,12,19-tetraazatricyclo[12.3.1.14,8]nonadeca-1(17),4(19),5,7,14(18),15-hexaene-2,2,13-trione (16.4 mg, 56%). ESI-MS m/z calc. 751.34033, found 752.3 (M+1)⁺; Retention time: 2.01 minutes; LC method A.

### Step 2: (11R)-6-(2,6-Dimethylphenyl)-12-[7-(2-hydroxyacetyl)-7-azaspiro[3.5]nonan-2-yl]-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 25)

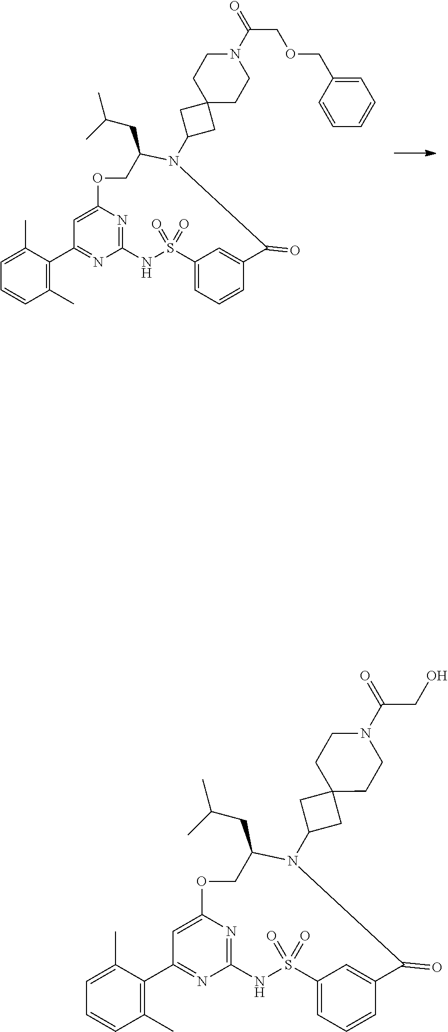 [SMILES: Cc1cccc(C)c1-c1cc2nc(n1)NS(=O)(=O)c1cccc(c1)C(=O)N(C1CC3(CCN(C(=O)COCc4ccccc4)CC3)C1)[C@H](CC(C)C)CO2 ; Cc1cccc(C)c1-c1cc2nc(n1)NS(=O)(=O)c1cccc(c1)C(=O)N(C1CC3(CCN(C(=O)CO)CC3)C1)[C@H](CC(C)C)CO2]

(11R)-12-[7-(2-Benzyloxyacetyl)-7-azaspiro[3.5]nonan-2-yl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (16.4 mg, 0.02181 mmol) and palladium on carbon (10 mg of 5% w/w, 0.004698 mmol) were combined in methanol (2 mL) under a balloon of hydrogen. The reaction was stirred for 1 h, filtered and purified by LC/MS utilizing a gradient of 1-99% acetonitrile in 5 mM aqueous HCl to yield (11R)-6-(2,6-dimethylphenyl)-12-[7-(2-hydroxyacetyl)-7-azaspiro[3.5]nonan-2-yl]-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (9.1 mg, 63%) ESI-MS m/z calc. 661.2934, found 662.3 (M+1)⁺; Retention time: 1.63 minutes (LC method A).

### Example 22: Preparation of Compound 26

### Step 1: 2-[(11R)-6-(2,6-Dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-N,N-dimethyl-7-azaspiro[3.5]nonane-7-carboxamide (Compound 26)

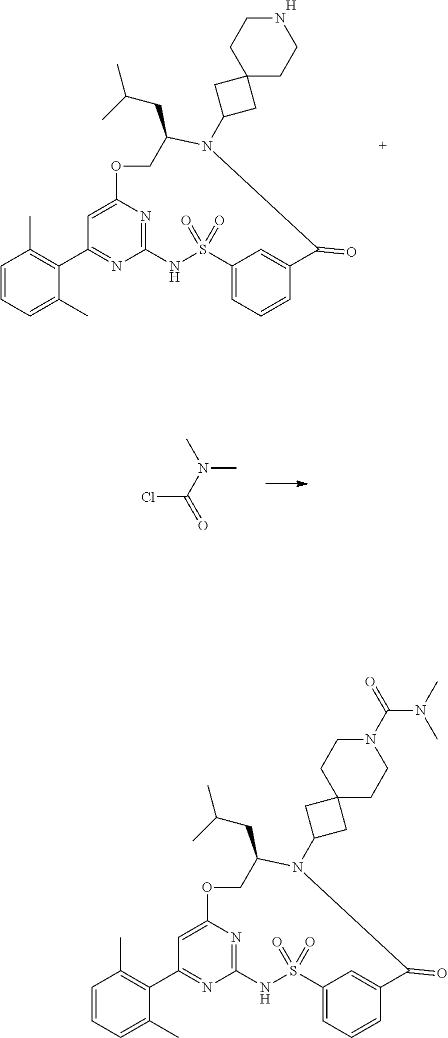

(11R)-12-(7-Azaspiro[3.5]nonan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (20 mg, 0.03124 mmol) was combined with N,N-dimethyl carbamoyl chloride (12 μL, 0.1308 mmol) and triethylamine (40 μL, 0.2870 mmol) in DCM (0.3 mL) and was stirred for 15 minutes at room temperature. After this time the reaction mixture was quenched with several drops of 1M HCl and partially concentrated. The resulting residue was dissolved in 1:1 methanol/DMSO, filtered and purified by reverse phase HPLC 1-99 ACN in water, HCl modifier, 15 min run) to give 2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-N,N-dimethyl-7-azaspiro[3.5]nonane-7-carboxamide (14 mg, 66%) ESI-MS m z calc. 674.325, found 675.7 (M+1)⁺; Retention time: 1.81 minutes; LC method A.

### Example 23: Preparation of Compound 27

### Step 1: 3-[[4-[(2R)-2-(Cyclobutylamino)-4-methyl-pentoxy]-6-(2,6-dimethylphenyl)pyrimidin-2-yl]sulfamoyl]benzoic acid

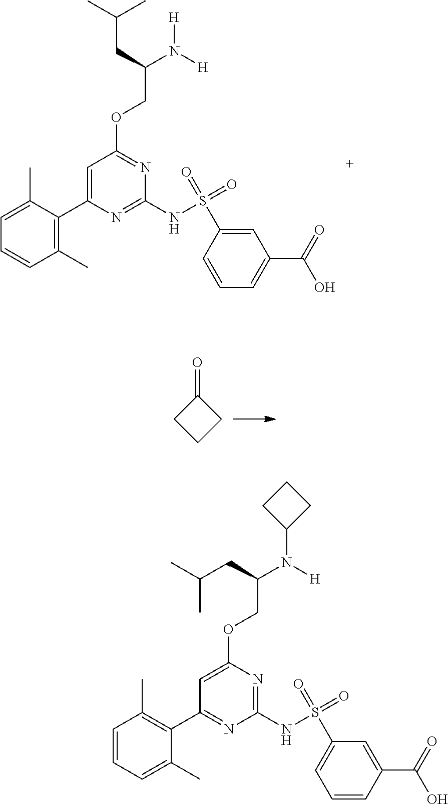 [SMILES: Cc1cccc(C)c1-c1cc(OC[C@H](N)CC(C)C)nc(NS(=O)(=O)c2cccc(C(=O)O)c2)n1 ; O=C1CCC1 ; Cc1cccc(C)c1-c1cc(OC[C@@H](CC(C)C)NC2CCC2)nc(NS(=O)(=O)c2cccc(C(=O)O)c2)n1]

To cyclobutanone (approximately 7.030 mg, 0.1003 mmol) was added a solution of 3-[[4-[(2R)-2-amino-4-methyl-pentoxy]-6-(2,6-dimethylphenyl)pyrimidin-2-yl]sulfamoyl]benzoic acid (50 mg, 0.1003 mmol) in NMP (0.2 mL) and dichloromethane (0.4 mL). After stirring at room temperature for 10 minutes, sodium triacetoxyborohydride (approximately 106.3 mg, 0.5015 mmol) was added. After stirring at room temperature for 30 minutes, the reaction mixture was filtered and purified by UV-triggered reverse-phase HPLC: Samples were purified using a reverse phase HPLC method using a Luna C₁₈ (2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. 3-[[4-[(2R)-2-(Cyclobutylamino)-4-methyl-pentoxy]-6-(2,6-dimethylphenyl)pyrimidin-2-yl]sulfamoyl]benzoic acid was obtained.

### Step 2: (11R)-12-cyclobutyl-6-(2,6-dimethylphenyl)-11-isobutyl-2,2-dioxo-9-oxa-21 6-thia-3,5,12,19-tetrazatricyclo[12.3.1.14,8]nonadeca-1(18),4(19),5,7,14,16-hexaen-13-one (Compound 27)

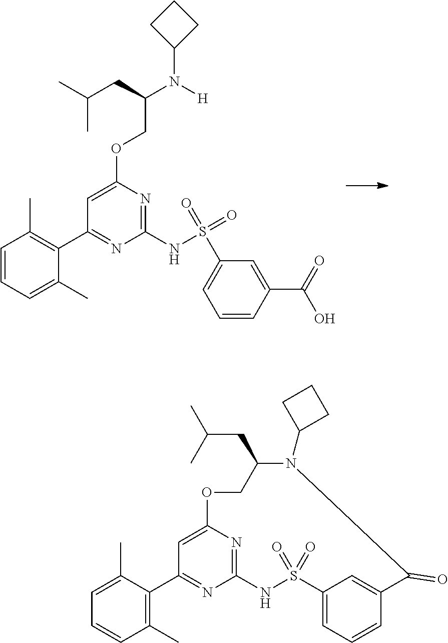 [SMILES: Cc1cccc(C)c1-c1cc(OC[C@@H](CC(C)C)NC2CCC2)nc(NS(=O)(=O)c2cccc(C(=O)O)c2)n1 ; Cc1cccc(C)c1-c1cc2nc(n1)NS(=O)(=O)c1cccc(c1)C(=O)N(C1CCC1)[C@H](CC(C)C)CO2]

3-[[4-[(2R)-2-(cyclobutylamino)-4-methyl-pentoxy]-6-(2,6-dimethylphenyl)pyrimidin-2-yl]sulfamoyl]benzoic acid was dissolved in DMF. HATU was added. After stirring at room temperature for 5 minutes, triethylamine was added. After 5 minutes of stirring, the product was isolated by UV-triggered reverse-phase HPLC: Gilson: Samples were purified using a reverse phase HPLC method using a Luna C₁₈ (2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. (11R)-12-cyclobutyl-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one was obtained. ESI-MS m/z calc. 534.2301, found 535.2 (M+1)⁺; Retention time: 1.91 minutes; LC method A.

### Example 24: Preparation of Compound 28

### Step 1: 3-[[4-[(2R)-2-[(3,3-Dimethylcyclobutyl)amino]-4-methyl-pentoxy]-6-(2,6-dimethylphenyl)pyrimidin-2-yl]sulfamoyl]benzoic acid (hydrochloride salt)

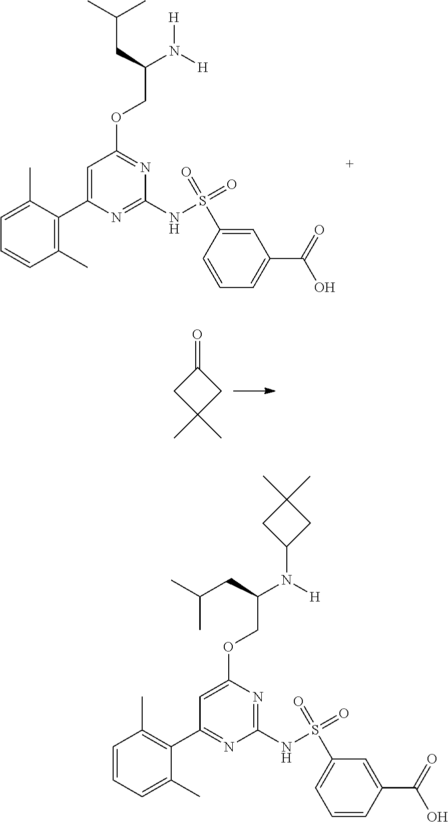

3-[[4-[(2R)-2-amino-4-methyl-pentoxy]-6-(2,6-dimethylphenyl)pyrimidin-2-yl]sulfamoyl]benzoic acid (hydrochloride salt) (40 mg, 0.07476 mmol) was combined with 3,3-dimethylcyclobutanone (approximately 22.01 mg, 0.2243 mmol) and acetic acid (approximately 35.92 mg, 34.02 μL, 0.5981 mmol) in DCE (0.4 mL) and stirred at room temperature for 20 minutes, at which point sodium cyanoborohydride (approximately 18.79 mg, 0.2990 mmol) was added. The reaction was stirred for 2 h at room temperature, and an additional portion of 3,3-dimethylcyclobutanone (approximately 22.01 mg, 0.2243 mmol) followed by sodium cyanoborohydride (approximately 18.79 mg, 0.2990 mmol) were added, and the reaction was stirred for an additional 1-6 h. At this point the reaction was quenched with 2 drops 1 M HCl, concentrated then dissolved in 1:1 DMSO/methanol, filtered and purified by reverse phase HPLC (1-70% ACN, HCl modifier) to give the corresponding 3-[[4-[(2R)-2-[(3,3-dimethylcyclobutyl)amino]-4-methyl-pentoxy]-6-(2,6-dimethylphenyl)pyrimidin-2-yl]sulfamoyl]benzoic acid (hydrochloride salt) (12 mg, 28%). ESI-MS m/z calc. 580.2719, found 581.5 (M+1)⁺; Retention time: 0.5 minutes; LC method D.

### Step 2: (11R)-12-(3,3-Dimethylcyclobutyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 28)

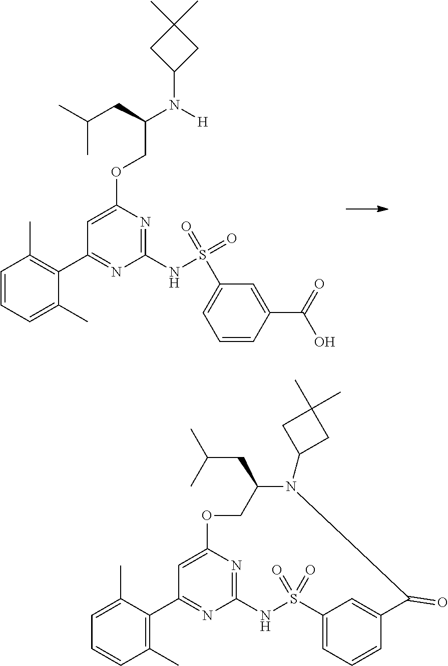

3-[[4-[(2R)-2-[(3,3-Dimethylcyclobutyl)amino]-4-methyl-pentoxy]-6-(2,6-dimethylphenyl)pyrimidin-2-yl]sulfamoyl]benzoic acid (hydrochloride salt) (11 mg, 0.01894 mmol) was combined with HATU (approximately 9.361 mg, 0.02462 mmol) in DMF (1 mL), and DIPEA (approximately 12.24 mg, 16.50 μL, 0.09470 mmol) was added. The reaction was stirred at room temperature for 1-2 hours, then filtered and purified by reverse phase HPLC (1-99% ACN in water, HCl modifier) to give (11R)-12-(3,3-dimethylcyclobutyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (6.4 mg, 60%) products after drying. ESI-MS m/z calc. 562.26135, found 563.5 (M+1)⁺; Retention time: 2.11 minutes; LC method A.

### Example 25: Preparation of Compound 29

### Step 1: 3-[[4-(2,6-Dimethylphenyl)-6-[(2R)-4-methyl-2-(2-oxaspiro[3.3]heptan-6-ylamino)pentoxy]pyrimidin-2-yl]sulfamoyl]benzoic acid

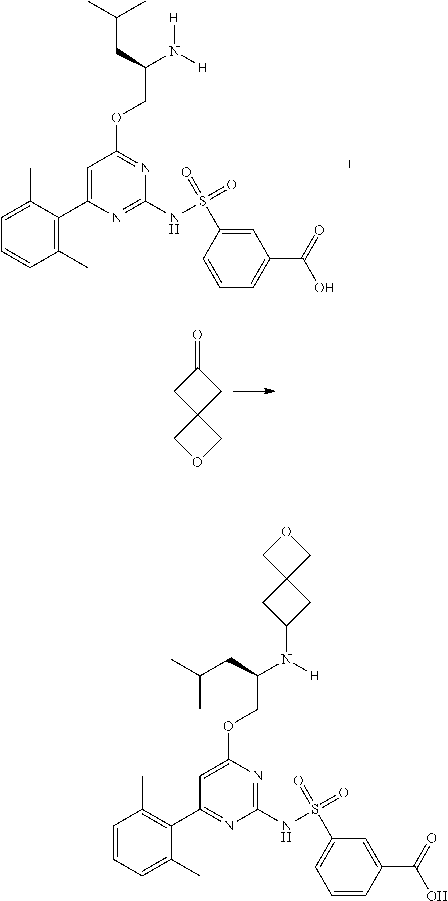 [SMILES: Cc1cccc(C)c1-c1cc(OC[C@H](N)CC(C)C)nc(NS(=O)(=O)c2cccc(C(=O)O)c2)n1 ; O=C1CC2(COC2)C1 ; Cc1cccc(C)c1-c1cc(OC[C@@H](CC(C)C)NC2CC3(COC3)C2)nc(NS(=O)(=O)c2cccc(C(=O)O)c2)n1]

3-[[4-[(2R)-2-Amino-4-methyl-pentoxy]-6-(2,6-dimethylphenyl)pyrimidin-2-yl]sulfamoyl]benzoic acid (hydrochloride salt) (50 mg, 0.09345 mmol), and 2-oxaspiro[3.3]heptan-6-one (approximately 31.44 mg, 0.2804 mmol) compound were combined in DCE (0.4 mL) with acetic acid (approximately 33.67 mg, 31.88 μL, 0.5607 mmol) and stirred at room temperature. After 30 minutes, sodium cyanoborohydride (approximately 23.49 mg, 0.3738 mmol) and 4 more equivalent of ketone were added and stirring at room temperature was continued for 4 hour. Another 4 equivalent of both reagents were added, and the reaction was stirred for 4 h. At this time the reaction mixture was quenched with 1 drop 1 M HCl, concentrated, then diluted with DMSO/methanol (1:1) and purified by reverse phase HPLC (1-99% ACN in water (no modifier) to give the 3-[[4-(2,6-dimethylphenyl)-6-[(2R)-4-methyl-2-(2-oxaspiro[3.3]heptan-6-ylamino)pentoxy]pyrimidin-2-yl]sulfamoyl]benzoic acid (13.1 mg, 24%) product. ESI-MS m/z calc. 594.2512, found 595.5 (M+1)⁺; Retention time: 0.43 minutes; LC method D.

### Step 2: (11R)-6-(2,6-Dimethylphenyl)-11-isobutyl-12-(2-oxaspiro[3.3]heptan-6-yl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 29)

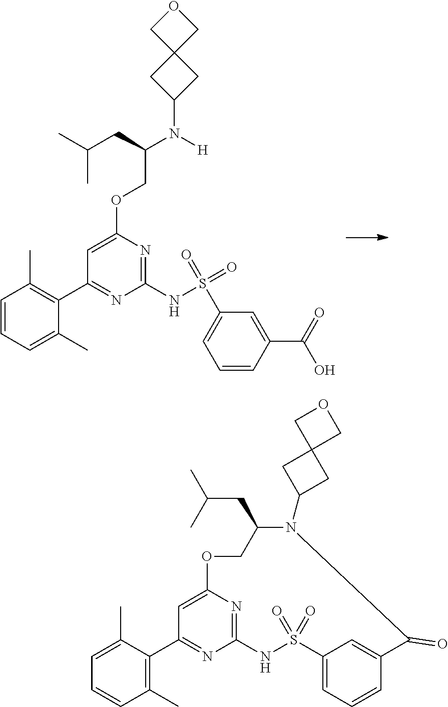

3-[[4-(2,6-dimethylphenyl)-6-[(2R)-4-methyl-2-(2-oxaspiro[3.3]heptan-6-ylamino)pentoxy]pyrimidin-2-yl]sulfamoyl]benzoic acid (15 mg, 0.02522 mmol) was combined with HATU (approximately 11.51 mg, 0.03026 mmol) in DMF (1 mL) and DIPEA (approximately 16.30 mg, 21.97 μL, 0.1261 mmol) was added. The reaction was stirred at room temperature for 30 minutes, then was filtered and purified by reverse phase HPLC (1-99% ACN in water without modifier, 15 min run). The compound was further purified by passing through a plug of silica, eluting with 50-100% ethyl acetate/hexanes to give (11R)-6-(2,6-dimethylphenyl)-11-isobutyl-12-(2-oxaspiro[3.3]heptan-6-yl)-2,2-dioxo-9-oxa-2λ⁶-thia-3,5,12,19-tetrazatricyclo[12.3.1.14,8]nonadeca-1(18),4(19),5,7,14,16-hexaen-13-one. ESI-MS m/z calc. 576.24066, found 577.5 (M+1)⁺; Retention time: 1.64 minutes; LC method A.

### Example 26: Preparation of Compound 30

### Step 1: 3-[[4-[(2R)-2-(Cyclopentylamino)-4-methyl-pentoxy]-6-(2,6-dimethylphenyl)pyrimidin-2-yl]sulfamoyl]benzoic acid

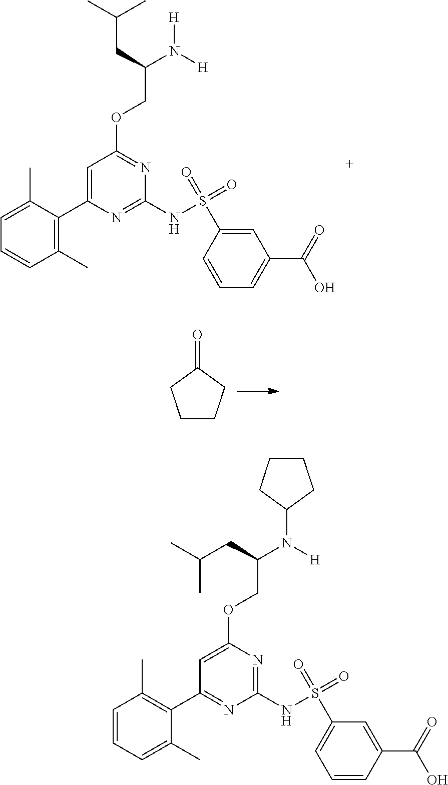 [SMILES: Cc1cccc(C)c1-c1cc(OC[C@H](N)CC(C)C)nc(NS(=O)(=O)c2cccc(C(=O)O)c2)n1 ; O=C1CCCC1 ; Cc1cccc(C)c1-c1cc(OC[C@@H](CC(C)C)NC2CCCC2)nc(NS(=O)(=O)c2cccc(C(=O)O)c2)n1]

3-[[4-[(2R)-2-amino-4-methyl-pentoxy]-6-(2,6-dimethylphenyl)pyrimidin-2-yl]sulfamoyl]benzoic acid (hydrochloride salt) (50 mg, 0.09345 mmol), and cyclopentanone (approximately 31.44 mg, 33.06 μL, 0.3738 mmol) compound were combined in DCE (0.4 mL) with acetic acid (approximately 33.67 mg, 31.88 μL, 0.5607 mmol) and stirred at room temperature. After 30 minutes, sodium cyanoborohydride (approximately 23.49 mg, 0.3738 mmol) was added, and stirring at room temperature was continued for 1 hour. An additional 4 equivalent of the ketone was added and the reaction was stirred for 1 hour. At this time the reaction mixture was quenched with 1 drop 1M HCl, concentrated, then diluted with DMSO/methanol (1:1) and purified by reverse phase HPLC (1-70% ACN in water HCl modifier [except as noted]) to give the 3-[[4-[(2R)-2-(cyclopentylamino)-4-methyl-pentoxy]-6-(2,6-dimethylphenyl)pyrimidin-2-yl]sulfamoyl]benzoic acid (38.6 mg, 73%) product. ESI-MS m z calc. 566.2563, found 567.5 (M+1)⁺; Retention time: 0.47 minutes; LC method D.

### Step 2: (11R)-12-Cyclopentyl-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 30)

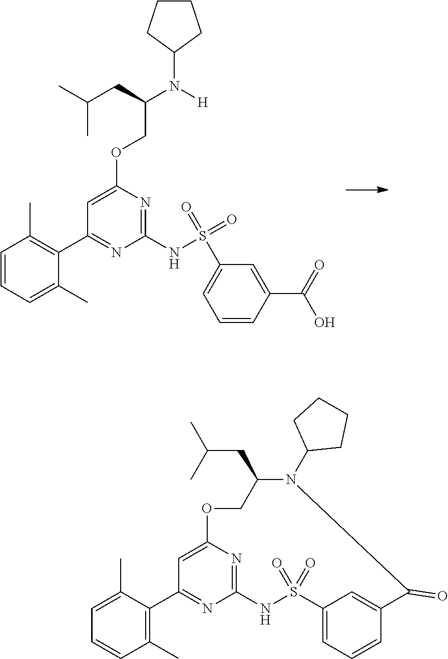 [SMILES: Cc1cccc(C)c1-c1cc(OC[C@@H](CC(C)C)NC2CCCC2)nc(NS(=O)(=O)c2cccc(C(=O)O)c2)n1 ; Cc1cccc(C)c1-c1cc2nc(n1)NS(=O)(=O)c1cccc(c1)C(=O)N(C1CCCC1)[C@H](CC(C)C)CO2]

3-[[4-[(2R)-2-(cyclopentylamino)-4-methyl-pentoxy]-6-(2,6-dimethylphenyl)pyrimidin-2-yl]sulfamoyl]benzoic acid (10 mg, 0.01765 mmol) was combined with HATU (25 mg, 0.06575 mmol) in DMSO (1 mL) and DIPEA (30 μL, 0.1722 mmol) was added. The reaction was stirred at room temperature for 18 h, then was filtered and purified by reverse phase HPLC (1-99% ACN with HCl modifier, 15 min run) to give the corresponding (11R)-12-cyclopentyl-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one. ESI-MS m/z calc. 548.2457, found 549.5 (M+1)⁺; Retention time: 1.98 minutes; LC method A.

### Example 27: Preparation of Compound 31

### Step 1: (11R)-6-(2,6-Dimethylphenyl)-11-isobutyl-12-(7-oxaspiro[3.5]nonan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 31)

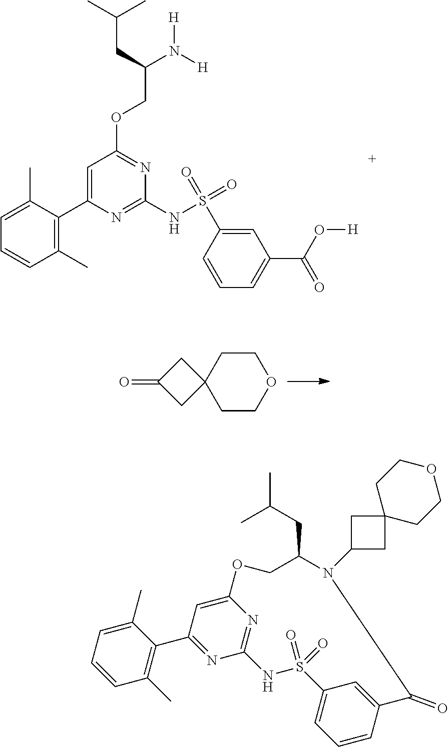 [SMILES: Cc1cccc(C)c1-c1cc(OC[C@H](N)CC(C)C)nc(NS(=O)(=O)c2cccc(C(=O)O)c2)n1 ; "+" ; O=C1CC2(CCOCC2)C1 ; Cc1cccc(C)c1-c1cc2nc(n1)NS(=O)(=O)c1cccc(c1)C(=O)N(C1CC3(CCOCC3)C1)[C@H](CC(C)C)CO2]

3-[[4-[(2R)-2-Amino-4-methyl-pentoxy]-6-(2,6-dimethylphenyl)pyrimidin-2-yl]sulfamoyl]benzoic acid (hydrochloride salt) (50 mg, 0.09345 mmol) was combined with 7-oxaspiro[3.5]nonan-2-one (approximately 26.20 mg, 0.1869 mmol) in dichloromethane. Sodium triacetoxyborohydride (approximately 59.43 mg, 0.2804 mmol) was added and the reaction was stirred at room temperature for an hour, at which time, if the reaction did not show complete conversion to the reductive amination product, an additional portion of sodium triacetoxyborohydride (approximately 59.43 mg, 0.2804 mmol) was added followed by an additional hour at room temperature. The reaction mixture was then added to a separatory funnel containing 50 mL 0.5 M HCl and 50 mL ethyl acetate. The layers were separated and the aqueous was extracted an additional 3×30 mL ethyl acetate. The combined organics were washed with brine, dried over sodium sulfate and concentrated.

The crude product was combined with HATU (approximately 56.84 mg, 0.1495 mmol) in DMF and DIPEA (approximately 60.38 mg, 81.37 μL, 0.4672 mmol) was added. After stirring 2 hours at room temperature the reaction mixture was diluted with 75 mL ethyl acetate and 100 mL 0.5 M HCl. The layers were separated, and the aqueous was extracted with an additional 50 mL ethyl acetate. The combined organics were washed 4×25 mL water, followed by brine, then dried over sodium sulfate and concentrated. The resulting crude material was dissolved in 1:1 DMSO/methanol, filtered, and purified by reverse phase HPLC (1-99% ACN in water, HCl modifier, 15 min run) to give as a white powder upon drying, the (11R)-6-(2,6-dimethylphenyl)-11-isobutyl-12-(7-oxaspiro[3.5]nonan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one (16 mg, 28%). ESI-MS m/z calc. 604.2719, found 605.5 (M+1)⁺; Retention time: 1.81 minutes; LC method A. ¹H NMR (400 MHz, DMSO) δ 13.15 (bs, 1H), 8.38 (s, 1H), 7.89 (s, 1H), 7.67 (s, 2H), 7.25 (s, 1H), 7.12 (s, 2H), 6.38 (s, 1H), 5.08 (d, J=10.7 Hz, 1H), 4.37 (s, 1H), 4.04 (t, J=8.8 Hz, 1H), 3.72 (s, 1H), 13.43-12.85 (m, 1H), 3.55 (t, J=5.2 Hz, 2H), 3.48 (t, J=5.2 Hz, 2H), 3.31-3.30 (m, 2H), 2.91-2.72 (m, 2H), 2.22-1.88 (m, 7H), 1.76-1.57 (m, 4H), 1.30 (s, 1H), 1.13 (t, J=11.9 Hz, 1H), 0.74 (d, J=6.6 Hz, 3H), 0.20 (s, 3H).

### Example 28: Preparation of Compound 32

### Step 1: (11R)-6-(2,6-dimethylphenyl)-11-isobutyl-2,2-dioxo-12-spiro[3.3]heptan-2-yl-9-oxa-2λ⁶-thia-3,5,12,19-tetrazatricyclo[12.3.1.14,8]nonadeca-1(18),4(19),5,7,14,16-hexaen-13-one (Compound 32)

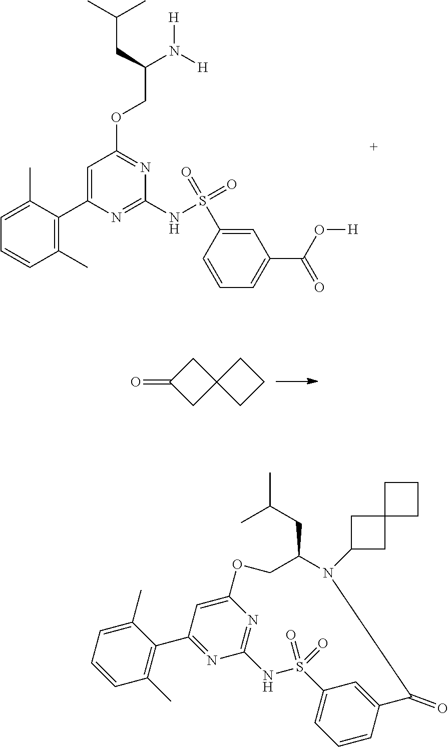 [SMILES: Cc1cccc(C)c1-c1cc(OC[C@H](N)CC(C)C)nc(NS(=O)(=O)c2cccc(C(=O)O)c2)n1 ; "+" ; O=C1CC2(CCC2)C1 ; Cc1cccc(C)c1-c1cc2nc(n1)NS(=O)(=O)c1cccc(c1)C(=O)N(C1CC3(CCC3)C1)[C@H](CC(C)C)CO2]

3-[[4-[(2R)-2-Amino-4-methyl-pentoxy]-6-(2,6-dimethylphenyl)pyrimidin-2-yl]sulfamoyl]benzoic acid (hydrochloride salt) (80 mg, 0.1495 mmol) was combined with spiro[3.3]heptan-2-one (approximately 32.94 mg, 0.2990 mmol) in dichloromethane. Sodium triacetoxyborohydride (approximately 63.37 mg, 0.2990 mmol) was added and the reaction was stirred at room temperature for an hour, at which time, if the reaction did not show complete conversion to the reductive amination product, an additional portion of sodium triacetoxyborohydride (approximately 63.37 mg, 0.2990 mmol) was added followed by an additional hour at room temperature. The reaction mixture was then added to a separatory funnel containing 50 mL 0.5 M HCl and 50 mL ethyl acetate. The layers were separated and the aqueous was extracted an additional 3×30 mL ethyl acetate. The combined organics were washed with brine, dried over sodium sulfate and concentrated.

The crude product was combined with HATU (approximately 113.7 mg, 0.2990 mmol) in DMF and DIPEA (approximately 96.61 mg, 130.2 μL, 0.7475 mmol) was added. After stirring 2 hours at room temperature the reaction mixture was diluted with 75 mL ethyl acetate and 100 mL 0.5 M HCl. The layers were separated, and the aqueous was extracted with an additional 50 mL ethyl acetate. The combined organics were washed 4×25 mL water, followed by brine, then dried over sodium sulfate and concentrated. The resulting crude material was dissolved in 1:1 DMSO/methanol, filtered, and purified by reverse phase HPLC (1-99% ACN in water, HCl modifier, 15 min run) to give as a white powder upon drying, the (11R)-6-(2,6-dimethylphenyl)-11-isobutyl-2,2-dioxo-12-spiro[3.3]heptan-2-yl-9-oxa-2λ⁶-thia-3,5,12,19-tetrazatricyclo[12.3.1.14,8]nonadeca-1(18),4(19),5,7,14,16-hexaen-13-one (39 mg, 44%). ESI-MS m/z calc. 574.26135, found 575.5 (M+1)⁺; Retention time: 2.15 minutes; LC method A. ¹H NMR (400 MHz, DMSO) δ 13.06 (s, 1H), 8.38 (s, 1H), 7.90 (s, 1H), 7.67 (s, 2H), 7.26 (t, J=7.8 Hz, 1H), 7.12 (d, J=7.9 Hz, 2H), 6.38 (s, 1H), 5.10 (dd, J=11.3, 4.3 Hz, 1H), 4.34 (t, J=10.9 Hz, 1H), 3.84 (p, J=9.5 Hz, 1H), 3.70 (d, J=11.6 Hz, 1H), 2.93 (t, J=9.8 Hz, 2H), 2.23 (q, J=7.9 Hz, 2H), 2.16-1.89 (m, 10H), 1.81 (p, J=7.9 Hz, 2H), 1.63 (t, J=12.0 Hz, 1H), 1.29 (s, 1H), 1.14 (dd, J=14.0, 10.0 Hz, 1H), 0.74 (d, J=6.7 Hz, 3H), 0.21 (d, J=6.2 Hz, 3H).

### Example 29: Preparation of Compound 33 and Compound 34

### Step 1: tert-Butyl 2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-6-azaspiro[3.4]octane-6-carboxylate

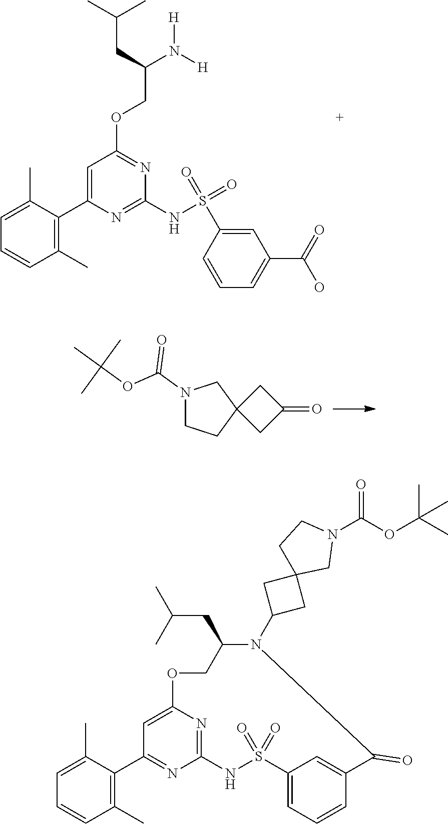 [SMILES: Cc1cccc(C)c1-c1cc(OC[C@H](N)CC(C)C)nc(NS(=O)(=O)c2cccc(C(=O)O)c2)n1 ; CC(C)(C)OC(=O)N1CCC2(CC(=O)C2)C1 ; Cc1cccc(C)c1-c1cc2nc(n1)NS(=O)(=O)c1cccc(c1)C(=O)N(C1CC3(CCN(C(=O)OC(C)(C)C)C3)C1)[C@H](CC(C)C)CO2]

3-[[4-[(2R)-2-Amino-4-methyl-pentoxy]-6-(2,6-dimethylphenyl)pyrimidin-2-yl]sulfamoyl]benzoic acid (hydrochloride salt) (150 mg, 0.2803 mmol) and tert-butyl 2-oxo-6-azaspiro[3.4]octane-6-carboxylate (approximately 126.3 mg, 0.5606 mmol) were combined in DCM (0.5 mL) and sodium triacetoxyborohydride (approximately 118.8 mg, 0.5606 mmol) (2 equiv) was added. After stirring for 30 minutes at room temperature, an additional portion of sodium triacetoxyborohydride (approximately 59.41 mg, 0.2803 mmol) (1 equiv) was added, followed by a final portion of sodium triacetoxyborohydride (approximately 59.41 mg, 0.2803 mmol) (1 equiv) after a further 30 minutes. The reaction was allowed to stir at room temperature for 30 minutes after the final addition then was added to a separatory funnel containing 20 mL of 0.5M HCl and 20 mL ethyl acetate. The layers were separated and the aqueous was extracted with an additional 2×10 mL ethyl acetate. The organics were combined, washed with brine and dried over sodium sulfate. The reaction mixture was concentrated and the crude product was combined with HATU (approximately 159.8 mg, 0.4204 mmol) in DMF (20 mL), and DIPEA (approximately 181.2 mg, 244.2 μL, 1.402 mmol) was added. The reaction mixture was stirred for 3 hours at room temperature then was poured into a separatory funnel containing 60 mL 0.5 M HCl and 60 mL ethyl acetate. The layers were separated and the aqueous was extracted an additional 2×40 mL ethyl acetate. The organics were combined, washed with water, brine, dried over sodium sulfate, filtered, and concentrated. The resulting crude material was purified by chromatography on silica gel, eluting with a gradient of 0-10% methanol in dichloromethane to give tert-butyl 2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-6-azaspiro[3.4]octane-6-carboxylate (130 mg, 67%). ESI-MS m/z calc. 689.3247, found 690.5 (M+1)⁺; Retention time: 0.81 minutes; LC method D.

### Step 2: (11R)-12-(6-Azaspiro[3.4]octan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one

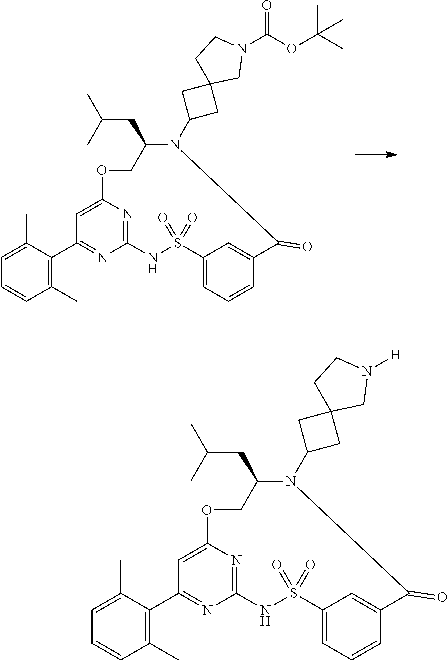 [SMILES: Cc1cccc(C)c1-c1cc2nc(n1)NS(=O)(=O)c1cccc(c1)C(=O)N(C1CC3(CCN(C(=O)OC(C)(C)C)C3)C1)[C@H](CC(C)C)CO2 ; Cc1cccc(C)c1-c1cc2nc(n1)NS(=O)(=O)c1cccc(c1)C(=O)N(C1CC3(CCNC3)C1)[C@H](CC(C)C)CO2]

The tert-Butyl 2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-6-azaspiro[3.4]octane-6-carboxylate (130 mg, 0.1884 mmol) was dissolved in dichloromethane (0.5 mL) and HCl (0.5 mL of 4 M, 2.000 mmol) in dioxane was added. The reaction mixture was stirred at room temperature for one hour. The reaction mixture was concentrated, then 0.5 mL hexanes and 0.5 mL dichloromethane were added and the reaction mixture was concentrated a second time and dried on high vac to give the corresponding (11R)-12-(6-azaspiro[3.4]octan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (110 mg, 93%) (as mixtures of syn and anti-cyclobutanones). ESI-MS m/z calc. 589.27, found 590.5 (M+1)⁺; Retention time: 0.52 minutes; LC method A.

### Step 3: (11R)-6-(2,6-dimethylphenyl)-11-isobutyl-12-(6-isopropyl-6-azaspiro[3.4]octan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one, (hydrochloride salt), diastereomer 1 (Compound 33), and (11R)-6-(2,6-dimethylphenyl)-11-isobutyl-12-(6-isopropyl-6-azaspiro[3.4]octan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt), diastereomer 2 (Compound 34)

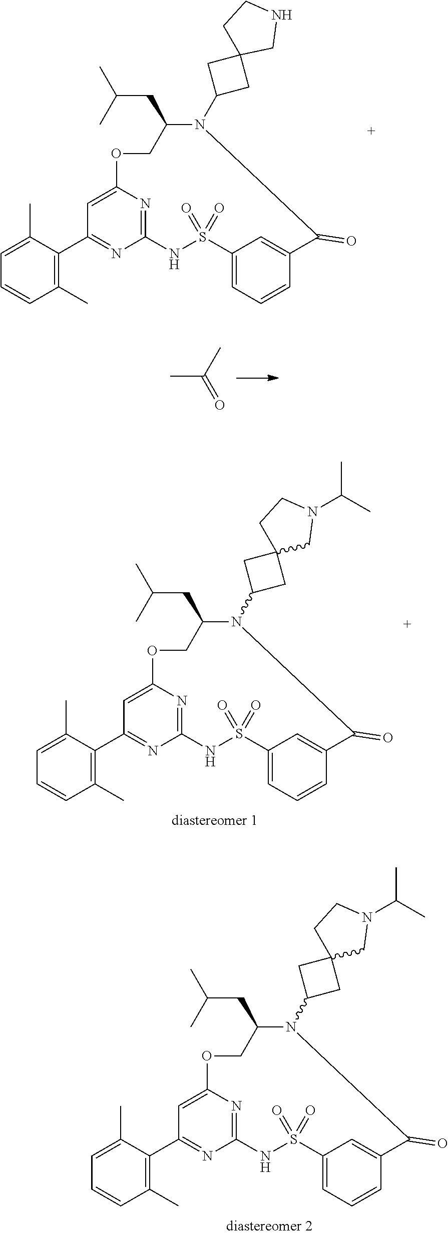

11R)-12-(6-Azaspiro[3.4]octan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (19 mg, 0.03034 mmol) was combined with acetone (9 μL, 0.1226 mmol) in dichloromethane (0.5 mL). Sodium triacetoxyborohydride (40 mg, 0.1887 mmol) was added, and the reaction was stirred at room temperature for 3 hours. The reaction mixture was then partially concentrated and re-dissolved in 1:1 DMSO/methanol and filtered. A reverse phase HPLC run of (1-99 ACN in water, HCl modifier, 30 minutes) only partially separated the cis/trans isomers. Fractions containing the partially separated cis/trans isomers were further purified separately by reverse phase HPLC (15-65ACN in water, HCl modifier) to give separately peak 1, (11R)-6-(2,6-dimethylphenyl)-11-isobutyl-12-(6-isopropyl-6-azaspiro[3.4]octan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt), diastereomer 1 (2.4 mg, 12%); ESI-MS m/z calc. 631.3192, found 632.6 (M+1)⁺; Retention time: 1.37 minutes and peak 2 (11R)-6-(2,6-dimethylphenyl)-11-isobutyl-12-(6-isopropyl-6-azaspiro[3.4]octan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) diastereomer 2 (2.8 mg, 13%) ESI-MS m/z calc. 631.3192, found 632.5 (M+1)⁺; Retention time: 1.4 minutes. (LC method A).

### Example 30: Preparation of Compound 35

### Step 1: tert-Butyl 3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutanecarboxylate

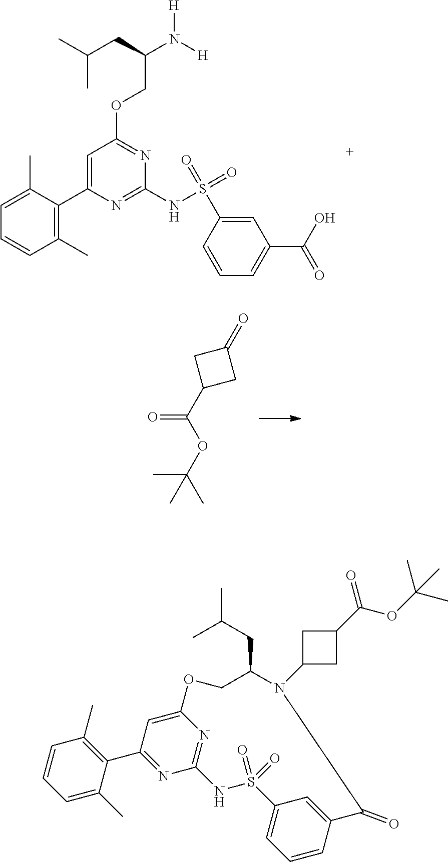

3-[[4-[(2R)-2-Amino-4-methyl-pentoxy]-6-(2,6-dimethylphenyl)pyrimidin-2-yl]sulfamoyl]benzoic acid (hydrochloride salt) (200 mg, 0.3738 mmol) was combined with tert-butyl 3-oxocyclobutanecarboxylate (approximately 127.2 mg, 0.7476 mmol) in DCM (0.5 mL). Sodium triacetoxyborohydride (approximately 237.6 mg, 1.121 mmol) was added, and the reaction was stirred for 1 hour at room temperature. Additional sodium triacetoxyborohydride (approximately 158.4 mg, 0.7476 mmol) was added and the reaction mixture was stirred for an additional 2 hours. The reaction mixture was then poured into a separatory funnel containing 0.5 M HCl, and ethyl acetate. The layers were separated and the aqueous was extracted three additional times with ethyl acetate. The organics were combined and washed with brine, dried over sodium sulfate, and concentrated. The resulting crude material was combined with HATU (approximately 284.3 mg, 0.7476 mmol) in DMF (15 mL), and DIEA (approximately 241.6 mg, 325.6 μL, 1.869 mmol) was added. The reaction mixture was stirred for 16 hours, then was poured into a separatory funnel containing ethyl acetate and 1M HCl. The layers were separated and the aqueous was extracted 3 additional times with ethyl acetate. The combined organics were washed with brine dried over sodium sulfate and concentrated. The compound was then dissolved in 1:1 DMSO/methanol, filtered, and purified by reverse phase HPLC (MeCN in water 1-99% HCl modifier) to give the corresponding tert-butyl 3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutanecarboxylate (188 mg, 79%). ESI-MS m/z calc. 634.28253, found 635.5 (M+1)⁺; Retention time: 0.79 minutes; LC method D.

### Step 2:3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutanecarboxylic acid, 70:30%, unknown absolute configuration, syn/anti mixture (Compound 35)

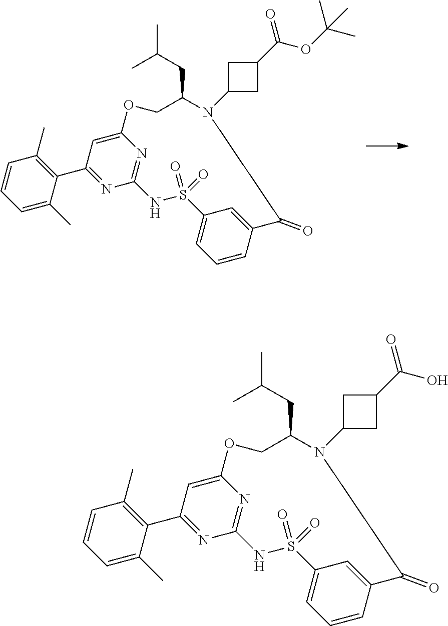 [SMILES: Cc1cccc(C)c1-c1cc2nc(n1)NS(=O)(=O)c1cccc(c1)C(=O)N(C1CC(C(=O)OC(C)(C)C)C1)[C@H](CC(C)C)CO2 ; Cc1cccc(C)c1-c1cc2nc(n1)NS(=O)(=O)c1cccc(c1)C(=O)N(C1CC(C(=O)O)C1)[C@H](CC(C)C)CO2]

tert-butyl 3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutanecarboxylate (188 mg, 0.2962 mmol) was dissolved in HCl (1.5 mL of 4 M, 6.000 mmol) and stirred at room temperature for 1 hour. The reaction mixture was then diluted with dichloromethane and concentrated. Hexanes were added and the reaction mixture was concentrated a second time to give as a slightly yellow solid, 3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutanecarboxylic acid (170 mg, 99%) ESI-MS m z calc. 578.2199, found 579.3 (M+1)⁺; Retention time: 0.62 minutes (mix of syn and anti-substituted cyclobutane), LC method D. A 8 mg portion of this material was further purified by reverse phase HPLC (1-99% ACN in water, HCl modifier, 15 min run) to give 3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutanecarboxylic acid diastereomer 1 (4 mg, 2%) ESI-MS m/z calc. 578.2199, found 579.3 (M+1)⁺; Retention time: 1.57 minutes (LC method A).

### Example 31: Preparation of Compound 36

### Step 1: (11R)-6-(2,6-dimethylphenyl)-11-isobutyl-12-[3-(4-methylpiperazine-1-carbonyl)cyclobutyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 36)

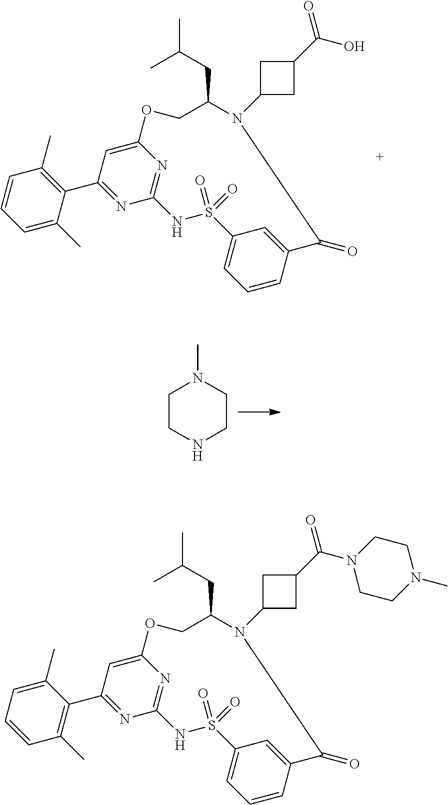

3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutanecarboxylic acid (15 mg, 0.02592 mmol) was combined with 1-methylpiperazine (approximately 5.192 mg, 0.05184 mmol) and HATU (approximately 19.71 mg, 0.05184 mmol) in DMF and DIPEA (approximately 16.75 mg, 22.57 μL, 0.1296 mmol) was added. The reaction was stirred at room temperature for 2 hours then was filtered and purified by reverse phase HPLC (1-99% ACN in water, HCl modifier, 15 min run) to give (11R)-6-(2,6-dimethylphenyl)-11-isobutyl-12-[3-(4-methylpiperazine-1-carbonyl)cyclobutyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (2.4 mg, 13%). ESI-MS m/z calc. 660.3094, found 661.6 (M+1)⁺; Retention time: 1.119 minutes; LC method A.

### Example 32: Preparation of Compound 37

### Step 1: 3-[[4-[(2R)-2-[[(3aR,6aS)-5-Methoxy-1,2,3,3a,4,5,6,6a-octahydropentalen-2-yl]amino]-4-methyl-pentoxy]-6-(2,6-dimethylphenyl)pyrimidin-2-yl]sulfamoyl]benzoic acid

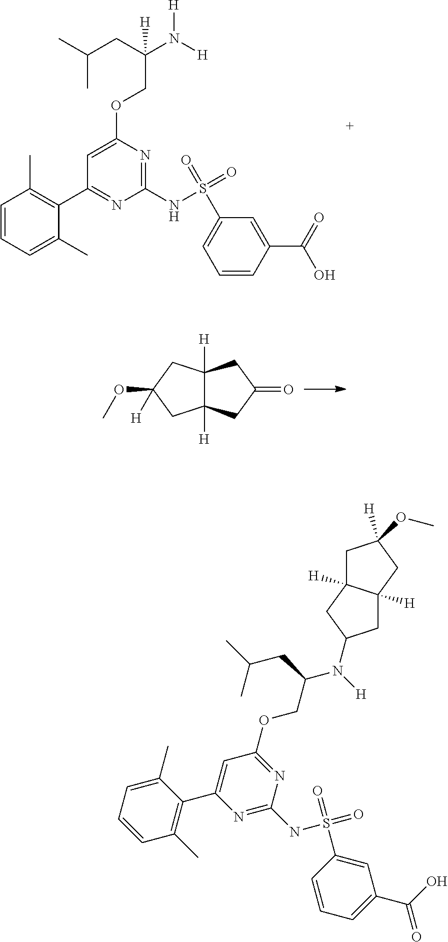 [SMILES: Cc1cccc(C)c1-c1cc(OC[C@H](N)CC(C)C)nc(NS(=O)(=O)c2cccc(C(=O)O)c2)n1 ; CO[C@H]1C[C@H]2CC(=O)C[C@H]2C1 ; CO[C@H]1C[C@H]2CC(N[C@@H](COc3cc(-c4c(C)cccc4C)nc(NS(=O)(=O)c4cccc(C(=O)O)c4)n3)CC(C)C)C[C@H]2C1]

In a 4 mL vial, to a stirred mixture of 3-[[4-[(2R)-2-amino-4-methyl-pentoxy]-6-(2,6-dimethylphenyl)pyrimidin-2-yl]sulfamoyl]benzoic acid (hydrochloride salt) (245 mg, 0.4579 mmol) and (3aS,6aR)-5-methoxy-3,3a,4,5,6,6a-hexahydro-1H-pentalen-2-one (75 mg, 0.4864 mmol) in anhydrous dichloromethane (1 mL) was added sodium triacetoxyborohydride (310 mg, 1.463 mmol). The vial was briefly purged with nitrogen and the mixture was stirred at ambient temperature for 20 h (overnight). Then methanol (0.2 mL) and water (0.2 mL) were added in that order, and the mixture was concentrated under reduced pressure. The residue was taken up in DMSO (3 mL), micro-filtered, and purified by reverse-phase HPLC, C₁₈ column, 1-99% acetonitrile in water over 15 min, HCl as modifier) to furnish 3-[[4-[(2R)-2-[[(3aR,6aS)-5-methoxy-1,2,3,3a,4,5,6,6a-octahydropentalen-2-yl]amino]-4-methyl-pentoxy]-6-(2,6-dimethylphenyl)pyrimidin-2-yl]sulfamoyl]benzoic acid (hydrochloride salt) (141 mg, 46%) as a white solid. ESI-MS m/z calc. 636.29816, found 637.2 (M+1)⁺; Retention time: 1.29 minutes; LC method A.

### Step 2: (11R)-12-[(3aR,6aS)-5-Methoxy-1,2,3,3a,4,5,6,6a-octahydropentalen-2-yl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 37)

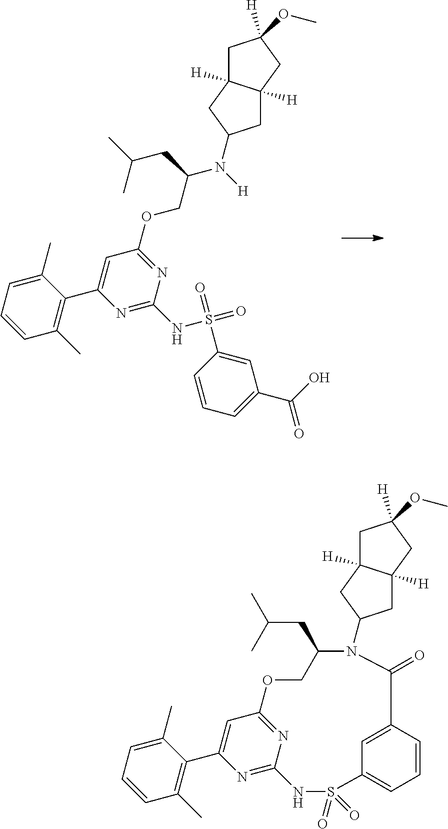 [SMILES: CO[C@H]1C[C@H]2CC(N[C@@H](COc3cc(-c4c(C)cccc4C)nc(NS(=O)(=O)c4cccc(C(=O)O)c4)n3)CC(C)C)C[C@H]2C1 ; CO[C@H]1C[C@H]2CC(N3C(=O)c4cccc(c4)S(=O)(=O)Nc4nc(cc(-c5c(C)cccc5C)n4)OC[C@H]3CC(C)C)C[C@H]2C1]

In a 4 mL vial, to a stirred solution of 3-[[4-[(2R)-2-[[(3aR,6aS)-5-methoxy-1,2,3,3a,4,5,6,6a-octahydropentalen-2-yl]amino]-4-methyl-pentoxy]-6-(2,6-dimethylphenyl)pyrimidin-2-yl]sulfamoyl]benzoic acid (hydrochloride salt) (50 mg, 0.07427 mmol) in anhydrous DMF (2.5 mL) were added 4-(6-cyano-2-methyl-7-oxo-4,8-dioxa-2,5-diazadec-5-en-3-ylidene)morpholin-4-ium hexafluorophosphate(V) (42 mg, 0.09807 mmol) (COMU) and DIEA (50 μL, 0.2871 mmol), in that order, Nitrogen gas was purged for 20 sec and capped. The reaction was stirred at ambient temperature for 14 h (overnight). The reaction mixture was poured into a stirred solution of water (150 mL) and HCl (35 mL of 1 M, 35.00 mmol). The mixture was diluted with DMSO (0.8 mL), micro-filtered, and purified by reverse-phase HPLC, C₁₈ column, 1-99% acetonitrile in water over 15 min, HCl as modifier) to furnish (11R)-12-[(3aR,6aS)-5-methoxy-1,2,3,3a,4,5,6,6a-octahydropentalen-2-yl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (14 mg, 30%) as white solid. ESI-MS m/z calc. 618.2876, found 619.1 (M+1)⁺; Retention time: 2.03 minutes; LCMS Method A.

### Example 33: Preparation of Compound 38 and Compound 39

### Step 1: tert-Butyl 3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]pyrrolidine-1-carboxylate

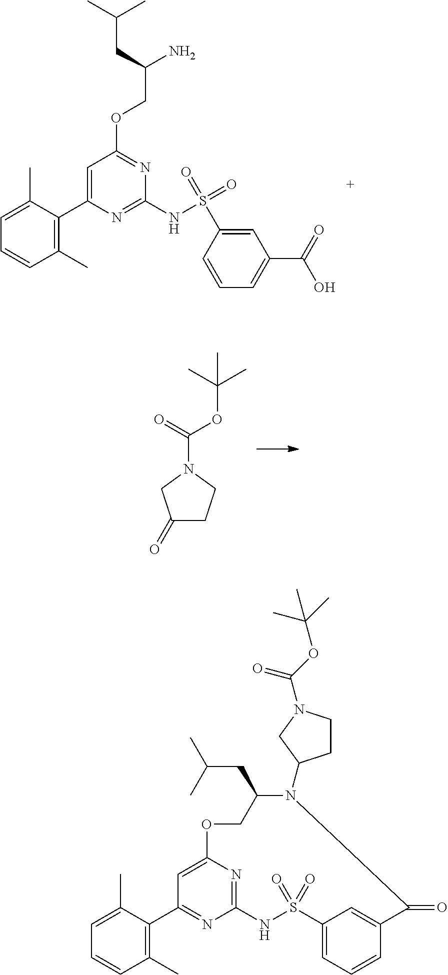 [SMILES: Cc1cccc(C)c1-c1cc(OC[C@H](N)CC(C)C)nc(NS(=O)(=O)c2cccc(C(=O)O)c2)n1 ; CC(C)(C)OC(=O)N1CCC(=O)C1 ; Cc1cccc(C)c1-c1cc2nc(n1)NS(=O)(=O)c1cccc(c1)C(=O)N(C1CCN(C(=O)OC(C)(C)C)C1)[C@H](CC(C)C)CO2]

3-[[4-[(2R)-2-amino-4-methyl-pentoxy]-6-(2,6-dimethylphenyl)pyrimidin-2-yl]sulfamoyl]benzoic acid (hydrochloride salt) (300 mg, 0.5607 mmol) was combined with tert-butyl 3-oxopyrrolidine-1-carboxylate (approximately 155.8 mg, 0.8411 mmol) in DCM (10 μL) and stirred at room temperature for one hour. A second portion of sodium triacetoxyborohydride (approximately 356.5 mg, 1.682 mmol) was then added and the reaction was stirred for an additional two hours. The reaction mixture was then partitioned between 0.5M HCl and ethyl acetate. The layers were separated and the aqueous was extracted an additional three times with ethyl acetate. The combined organics were washed with brine, dried over sodium sulfate and concentrated. The resulting material was dissolved in 5 mL DMF and added dropwise to a stirring solution of COMU (approximately 480.1 mg, 1.121 mmol) and DIPEA (approximately 434.8 mg, 586.0 μL, 3.364 mmol) in sufficient DMF to give a final concentration of 0.01 M. The reaction mixture was then stirred at room temperature for 16 hours. After this time the reaction mixture was partitioned between 1M HCl and ethyl acetate. The layers were separated and the aqueous was extracted an additional 3×with ethyl acetate. The combined organics were washed with brine, dried over sodium sulfate, and concentrated. The compound was purified by chromatography on silica gel (0-100 ethyl acetate in hexanes) to give tert-butyl 3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]pyrrolidine-1-carboxylate (101 mg, 28%). ESI-MS m/z calc. 649.2934, found 650.5 (M+1)⁺; Retention time: 0.76 minutes; LC method D.

### Step 2: (11R)-6-(2,6-Dimethylphenyl)-11-isobutyl-2,2-dioxo-12-pyrrolidin-3-yl-9-oxa-2λ⁶-thia-3,5,12,19-tetrazatricyclo[12.3.1.14,8]nonadeca-1(18),4(19),5,7,14,16-hexaen-13-one, diastereomer 1, and (11R)-6-(2,6-dimethylphenyl)-11-isobutyl-2,2-dioxo-12-pyrrolidin-3-yl-9-oxa-2λ⁶-thia-3,5,12,19-tetrazatricyclo[12.3.1.14,8]nonadeca-1(18),4(19),5,7,14,16-hexaen-13-one (hydrochloride salt), diastereomer 2

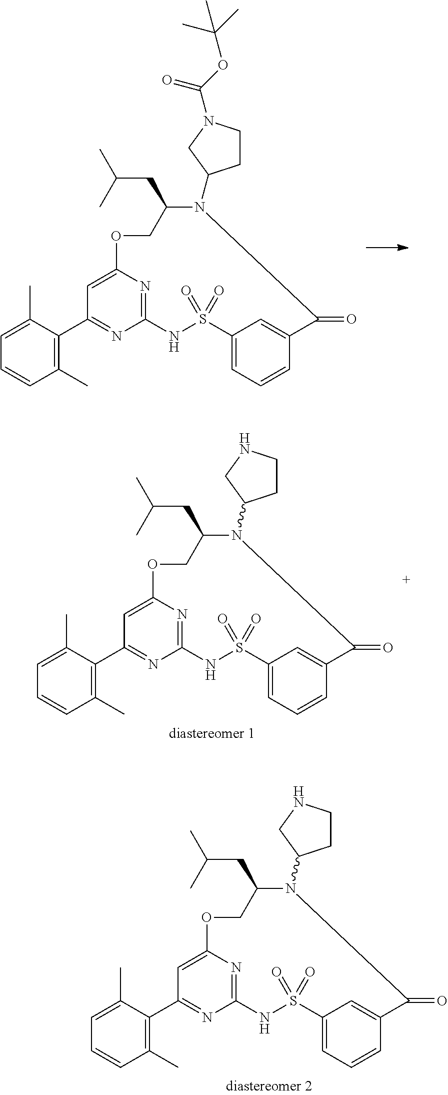 [SMILES: Cc1cccc(C)c1-c1cc2nc(n1)NS(=O)(=O)c1cccc(c1)C(=O)N(C1CCN(C(=O)OC(C)(C)C)C1)[C@H](CC(C)C)CO2 ; Cc1cccc(C)c1-c1cc2nc(n1)NS(=O)(=O)c1cccc(c1)C(=O)N(C1CCNC1)[C@H](CC(C)C)CO2 ; Cc1cccc(C)c1-c1cc2nc(n1)NS(=O)(=O)c1cccc(c1)C(=O)N(C1CCNC1)[C@H](CC(C)C)CO2]

tert-Butyl 3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]pyrrolidine-1-carboxylate (153 mg, 0.2355 mmol) was dissolved in dichloromethane (1 mL), and HCl (600 μL of 4 M, 2.400 mmol) was added. The reaction mixture was stirred for 20 minutes at room temperature then the reaction mixture was evaporated. The resulting material was purified by reverse phase (1-99% MeOH in water, HCl modifier, 30 min run with shallow initial gradient) to give the two diastereomers separately (absolute configuration unknown), (11R)-6-(2,6-dimethylphenyl)-11-isobutyl-2,2-dioxo-12-pyrrolidin-3-yl-9-oxa-2λ⁶-thia-3,5,12,19-tetrazatricyclo[12.3.1.14,8]nonadeca-1(18),4(19),5,7,14,16-hexaen-13-one (hydrochloride salt), diastereomer 1 (30 mg, 22%); ESI-MS m/z calc. 549.24097, found 550.5 (M+1)⁺; Retention time: 0.47 minutes, LCMS method A; and (11R)-6-(2,6-dimethylphenyl)-11-isobutyl-2,2-dioxo-12-pyrrolidin-3-yl-9-oxa-2λ⁶-thia-3,5,12,19-tetrazatricyclo[12.3.1.14,8]nonadeca-1(18),4(19),5,7,14,16-hexaen-13-one (hydrochloride salt) diastereomer 2 (13 mg, 9%); ESI-MS m/z calc. 549.24097, found 550.5 (M+1)⁺; Retention time: 0.49 minutes (LC method A).

### Step 3: propan-2-yl 3-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]pyrrolidine-1-carboxylate, diastereomer 1 (Compound 38), and propan-2-yl 3-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]pyrrolidine-1-carboxylate, diastereomer 2 (Compound 39)

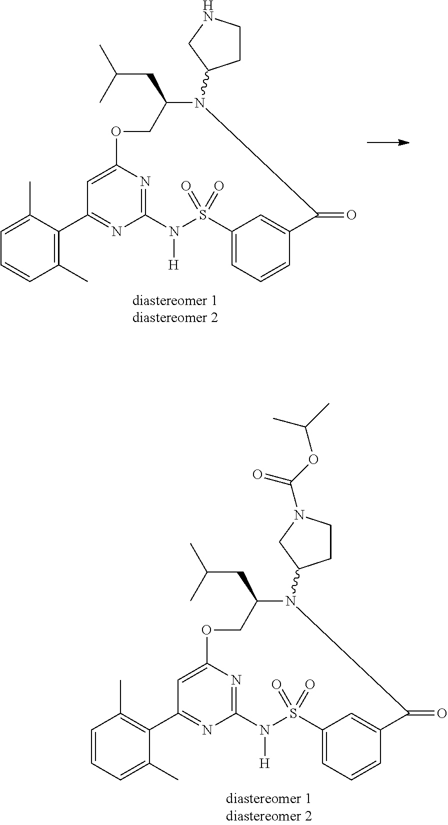 [SMILES: Cc1cccc(C)c1-c1cc2nc(n1)NS(=O)(=O)c1cccc(c1)C(=O)N(C1CCNC1)[C@H](CC(C)C)CO2 ; Cc1cccc(C)c1-c1cc2nc(n1)NS(=O)(=O)c1cccc(c1)C(=O)N(C1CCN(C(=O)OC(C)C)C1)[C@H](CC(C)C)CO2]

Each previously separated diastereomer 1 and 2 was reacted in a separate vial to give the corresponding pure diastereomeric product of unknown configuration at the pyrrolidine ring. (11R)-6-(2,6-Dimethylphenyl)-11-isobutyl-2,2-dioxo-12-pyrrolidin-3-yl-9-oxa-2λ⁶-thia-3,5,12,19-tetrazatricyclo[12.3.1.14,8]nonadeca-1(18),4(19),5,7,14,16-hexaen-13-one (hydrochloride salt) (10 mg, 0.01706 mmol) was combined in DCM (0.5 mL) with isopropyl chloroformate (approximately 17.06 μL of 2 M, 0.03412 mmol) (in toluene). DIPEA (approximately 11.02 mg, 14.85 μL, 0.08530 mmol) was added and the reaction mixture was stirred for 30 minutes at room temperature. The reaction mixture was then quenched with several drops of 1M HCl, partially concentrated, then diluted with 1:1 DMSO/methanol, and filtered. After purification by reverse phase HPLC (1-99% ACN in water, HCl modifier, 15 min run) the products were obtained as a white solid upon drying: Diastereomer 1, propan-2-yl 3-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]pyrrolidine-1-carboxylate (8 mg, 74%); ESI-MS m/z calc. 635.2778, found 636.5 (M+1)⁺; Retention time: 1.83 minutes; (LC method A), and diastereomer 2, propan-2-yl 3-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]pyrrolidine-1-carboxylate (4.5 mg, 63.82%); ESI-MS m/z calc. 635.2778, found 636.5 (M+1)⁺; Retention time: 1.87 minutes; LC method A.

### Example 34: Preparation of Compound 40

### Step 1: 3-[[4-(2,6-Dimethylphenyl)-6-[(2R)-2-[[3-(hydroxymethyl)cyclobutyl]amino]-4-methyl-pentoxy]pyrimidin-2-yl]sulfamoyl]benzoic acid

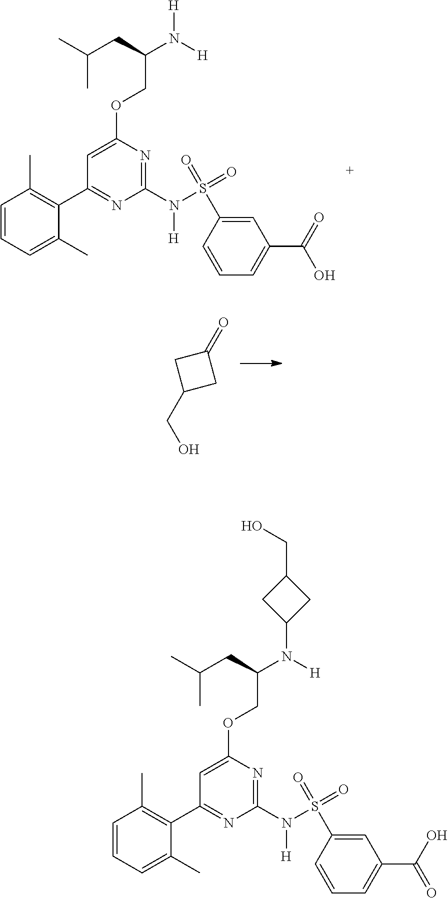 [SMILES: Cc1cccc(C)c1-c1cc(OC[C@H](N)CC(C)C)nc(NS(=O)(=O)c2cccc(C(=O)O)c2)n1 ; O=C1CC(CO)C1 ; Cc1cccc(C)c1-c1cc(OC[C@@H](CC(C)C)NC2CC(CO)C2)nc(NS(=O)(=O)c2cccc(C(=O)O)c2)n1]

3-[[4-[(2R)-2-Amino-4-methyl-pentoxy]-6-(2,6-dimethylphenyl)pyrimidin-2-yl]sulfamoyl]benzoic acid (40 mg, 0.08023 mmol) and 3-(hydroxymethyl)cyclobutanone (approximately 24.10 mg, 0.2407 mmol) were combined in DCE with acetic acid (approximately 38.54 mg, 36.50 μL, 0.6418 mmol) and stirred at room temperature for 20 minutes. Sodium cyanoborohydride (approximately 20.17 mg, 0.3209 mmol) was added and the reaction was stirred at room temperature for 1 hour. An additional portion of 3-(hydroxymethyl)cyclobutanone (approximately 24.10 mg, 0.2407 mmol) was added and the reaction was stirred for an additional 3 hours at room temperature. The reaction mixture was quenched with several drops of water, and partially concentrated. The reaction mixture was then re-dissolved in 1 mL 1:1 DMSO/methanol, then filtered and purified by reverse phase HPLC (1-70% ACN in water, HCl modifier) on a 15 min run. The fractions containing product were concentrated to give as a white solid, 3-[[4-(2,6-dimethylphenyl)-6-[(2R)-2-[[3-(hydroxymethyl)cyclobutyl]amino]-4-methyl-pentoxy]pyrimidin-2-yl]sulfamoyl]benzoic acid (hydrochloride salt) (20.3 mg, 41%); ESI-MS m/z calc. 582.2512, found 583.5 (M+1)⁺; Retention time: 0.43 minutes; LC method D.

### Step 2: (11R)-6-(2,6-Dimethylphenyl)-12-[3-(hydroxymethyl)cyclobutyl]-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 40)

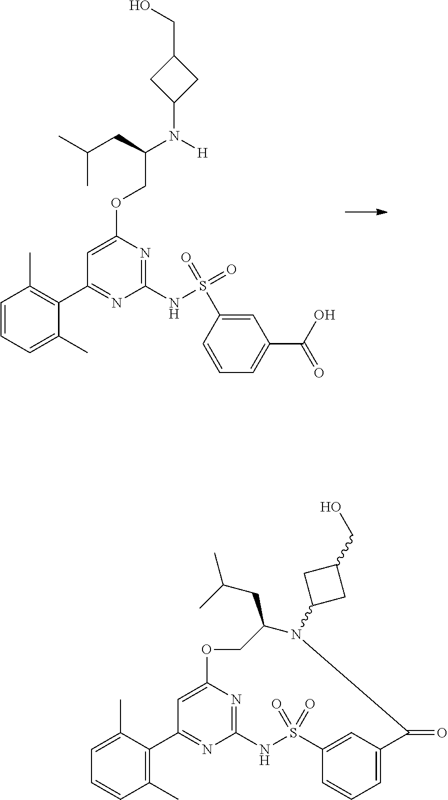 [SMILES: Cc1cccc(C)c1-c1cc(OC[C@@H](CC(C)C)NC2CC(CO)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC(CO)C1)[C@H](CC(C)C)CO2]

3-[[4-(2,6-dimethylphenyl)-6-[(2R)-2-[[3-(hydroxymethyl)cyclobutyl]amino]-4-methyl-pentoxy]pyrimidin-2-yl]sulfamoyl]benzoic acid (hydrochloride salt) (20 mg, 0.03230 mmol) was combined with HATU (approximately 15.97 mg, 0.04199 mmol) in DMF (1 mL) and DIPEA (approximately 20.87 mg, 28.13 μL, 0.1615 mmol) was added. The reaction mixture was then stirred at room temperature for 1 h. The reaction was filtered and purified by reverse phase HPLC (1-99% ACN in water, HCl modifier, 15 min run) to give (11R)-6-(2,6-dimethylphenyl)-12-[3-(hydroxymethyl)cyclobutyl]-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (single isomer of unknown cyclobutane configuration, 2 mg, 11%). ESI-MS m/z calc. 564.24066, found 565.5 (M+1)⁺; Retention time: 1.51 minutes; LC method A.

### Example 35: Preparation of Compound 41

### Step 1: Methyl 3-[[4-chloro-6-(2,6-dimethylphenyl)pyrimidin-2-yl]-(methoxymethyl)sulfamoyl]benzoate

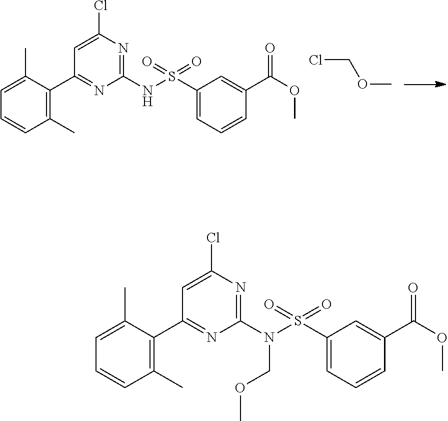

To a solution of methyl 3-[[4-chloro-6-(2,6-dimethylphenyl)pyrimidin-2-yl]sulfamoyl]benzoate (68.5 g, 158.60 mmol) in DMF (400 mL) at 0° C. was added potassium carbonate (44 g, 318.37 mmol) and chloro(methoxy)methane (13.992 g, 13.2 mL, 173.78 mmol). The reaction was stirred at room temperature for 1 h. Water (800 mL) was added and the product was extracted with DCM (3×150 mL). Combined organic layers were washed with a 1:1 mix of water and brine (4×200 mL), and then brine (1×150 mL). The resulting combined organic layers were dried over sodium sulfate, filtered and concentrated under reduced pressure. Afforded methyl 3-[[4-chloro-6-(2,6-dimethylphenyl)pyrimidin-2-yl]-(methoxymethyl)sulfamoyl]benzoate (80.4 g, 90%) as a brown oil. ESI-MS m/z calc. 475.09686, found 476.2 (M+1)⁺; Retention time: 2.06 minutes; LC method X.

### Step 2: 3-[[4-Chloro-6-(2,6-dimethylphenyl)pyrimidin-2-yl]-(methoxymethyl)sulfamoyl]benzoic acid

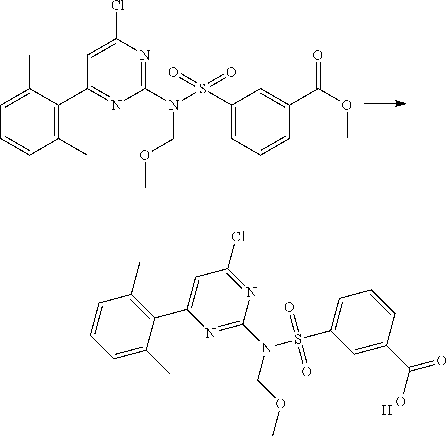

A mixture of methyl 3-[[4-chloro-6-(2,6-dimethylphenyl)pyrimidin-2-yl]-(methoxymethyl)sulfamoyl]benzoate (47.89 g, 80.698 mmol) in THE (475 mL) and water (475 mL) was treated with lithium hydroxide hydrate (5.07 g, 120.82 mmol) and it was stirred at room temperature for 4 hours. Most of the THF was removed under reduced pressure, and the remaining aqueous layer was acidified to a pH of about 2-3 using 1N aqueous HCl (250 ml). The product was extracted with ethyl acetate (3×450 mL). The combined organic layers were washed with brine (100 mL), dried over sodium sulfate, filtered and concentrated under reduced pressure. The resulting sticky solid was triturated twice in ethyl acetate (100 ml and 75 ml) to afford 3-[[4-chloro-6-(2,6-dimethylphenyl)pyrimidin-2-yl]-(methoxymethyl)sulfamoyl]benzoic acid (26.045 g, 65%) as a white solid. ¹H NMR (400 MHz, DMSO-d₆) δ 13.37 (br. s., 1H), 8.48 (s, 1H), 8.20-8.10 (m, 2H), 7.61 (t, J=7.8 Hz, 1H), 7.44 (s, 1H), 7.28-7.20 (m, 1H), 7.10 (d, J=7.6 Hz, 2H), 5.61 (s, 2H), 3.30 (s, 3H), 1.84 (s, 6H). ESI-MS m/z calc. 461.0812, found 462.1 (M+1)⁺; Retention time: 4.32 minutes; LC method Y.

### Step 3: 3-[[4-[(2R)-2-Amino-4-methyl-pentoxy]-6-(2,6-dimethylphenyl)pyrimidin-2-yl]-(methoxymethyl)sulfamoyl]benzoic acid

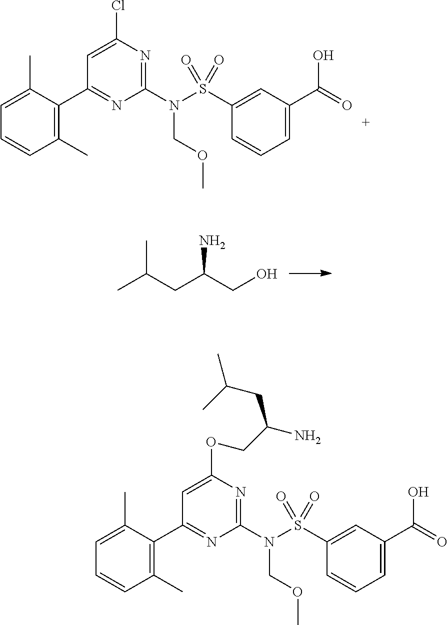

In a reaction vial, 3-[[4-chloro-6-(2,6-dimethylphenyl)pyrimidin-2-yl]-(methoxymethyl)sulfamoyl]benzoic acid (2.6 g, 5.629 mmol), (2R)-2-amino-4-methyl-pentan-1-ol (725 μL, 5.673 mmol), and sodium tert-butoxide (1.75 g, 18.21 mmol) were combined in THE (7 mL) and stirred at room temperature for 2 h. The reaction was diluted with ethyl acetate and washed with a 1M HCl solution. The organics were further washed with brine, dried over sodium sulfate and evaporated. The crude material was recrystallized from ethyl acetate to provide the product as a white solid 3-[[4-[(2R)-2-amino-4-methyl-pentoxy]-6-(2,6-dimethylphenyl)pyrimidin-2-yl]-(methoxymethyl)sulfamoyl]benzoic acid (hydrochloride salt)(1.95 g, 60%) ESI-MS m/z calc. 542.2199, found 543.3 (M+1)⁺; Retention time: 1.4 minutes (LC method A).

### Step 4: (11R)-6-(2,6-Dimethylphenyl)-11-isobutyl-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one

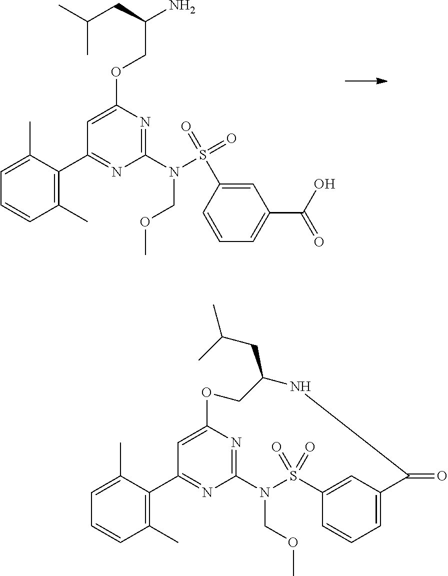 [SMILES: COCN(c1nc(OC[C@H](N)CC(C)C)cc(-c2c(C)cccc2C)n1)S(=O)(=O)c1cccc(C(=O)O)c1 ; COCN1c2nc(cc(-c3c(C)cccc3C)n2)OC[C@@H](CC(C)C)NC(=O)c2cccc(c2)S1(=O)=O]

3-[[4-[(2R)-2-amino-4-methyl-pentoxy]-6-(2,6-dimethylphenyl)pyrimidin-2-yl]-(methoxymethyl)sulfamoyl]benzoic acid (hydrochloride salt) (797 mg, 1.376 mmol) was dissolved in DMF (6 mL) and added to a solution of HATU (640.2 mg, 1.684 mmol) and triethylamine (766 μL, 5.496 mmol) in DMF (7 mL). The reaction was stirred at room temperature for 20 min. The reaction mixture was poured into water (20 mL) and the resulting solid was collected via filtration. The solids were dissolved in ethyl acetate and washed with a 1M HCl solution, then brine. The organics were dried over sodium sulfate and evaporated to give (11R)-6-(2,6-dimethylphenyl)-11-isobutyl-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (720 mg, 100%) ESI-MS m/z calc. 524.20935, found 525.3 (M+1)⁺; Retention time: 0.77 minutes; LC method D.

### Step 5: (11R)-6-(2,6-Dimethylphenyl)-12-(1,1-dioxothietan-3-yl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 41)

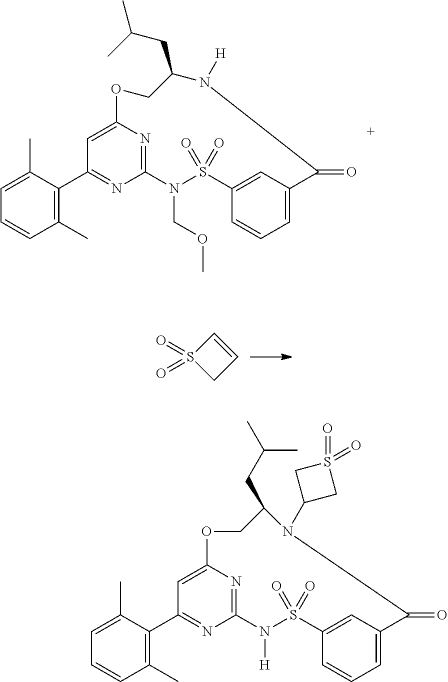 [SMILES: COCN1c2nc(cc(-c3c(C)cccc3C)n2)OC[C@@H](CC(C)C)NC(=O)c2cccc(c2)S1(=O)=O ; O=S1(=O)C=CC1 ; Cc1cccc(C)c1-c1cc2nc(n1)NS(=O)(=O)c1cccc(c1)C(=O)N(C1CS(=O)(=O)C1)[C@H](CC(C)C)CO2]

(11R)-6-(2,6-Dimethylphenyl)-11-isobutyl-3-(methoxymethyl)-2,2-dioxo-9-oxa-2λ⁶, thia-3,5,12,19-tetrazatricyclo[12.3.1.14,8]nonadeca-1(18),4(19),5,7,14,16-hexaen-13-one (10 mg, 0.01906 mmol) was combined with 2H-thiete 1,1-dioxide (3 mg, 0.02881 mmol) and potassium carbonate (4 mg, 0.02894 mmol) in DMF (0.5 mL) and stirred for two hours at room temperature. More sodium hydride (1.3 mg, 0.03250 mmol) was added. After one minute, the reaction mixture was quenched into 1M HCl, then it was extracted 3×with ethyl acetate. The combined organics were washed with brine, dried over sodium sulfate, filtered, and concentrated. The resulting product was dissolved in DCM (0.3 mL) and TFA (0.3 mL, 3.894 mmol) was added. The reaction was stirred for 15 minutes at room temperature. The reaction mixture was concentrated under reduced pressure and the resulting crude material was dissolved in 1:1 DMSO/methanol, filter4ed and purified by reverse phase HPLC (1-99% ACN in water, HCl modifier, 15 min run) to give (11R)-6-(2,6-dimethylphenyl)-12-(1,1-dioxothietan-3-yl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one. ESI-MS m/z calc. 584.17633, found 585.4 (M+1)⁺; Retention time: 1.5 minutes; LC method A.

### Example 36: Preparation of Compound 42

### Step 1: (2R)-2-[(3-Benzyloxycyclobutyl)amino]-4-methyl-pentan-1-ol

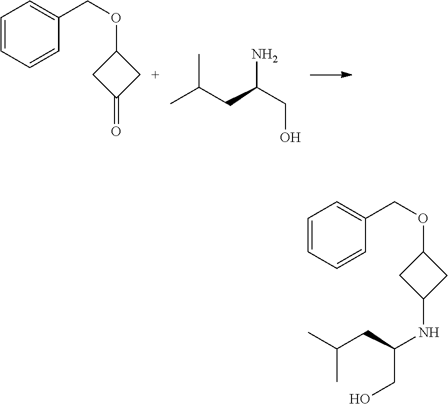

Into a solution of (2R)-2-amino-4-methyl-pentan-1-ol (2.0 g, 17.066 mmol) in anhydrous DCE (25 mL) was added 3-benzyloxycyclobutanone (2.389 g, 13.558 mmol). The reaction was stirred at room temperature for 30 minutes. Sodium triacetoxyborohydride (6.32 g, 29.820 mmol) was added to the reaction, and then it was stirred at room temperature overnight. The reaction was poured into 2 N sodium carbonate (30 mL). The reaction was extracted with DCM (3×30 mL). The combined organic layers were dried over anhydrous sodium sulfate and concentrated under vacuum. The residue was purified by silica gel chromatography using 0 to 10% methanol in DCM (buffered with 0.2% ammonium hydroxide) to furnish (2R)-2-[(3-benzyloxycyclobutyl)amino]-4-methyl-pentan-1-ol (3.379 g, 69%) as a clear oil. ESI-MS m/z calc. 277.2042, found 278.3 (M+1)⁺; Retention time: 3.68 minutes; LC method S.

### Step 2: 3-[[4-[(2R)-2-[(3-Benzyloxycyclobutyl)amino]-4-methyl-pentoxy]-6-(2,6-dimethylphenyl)pyrimidin-2-yl]sulfamoyl]benzoic acid

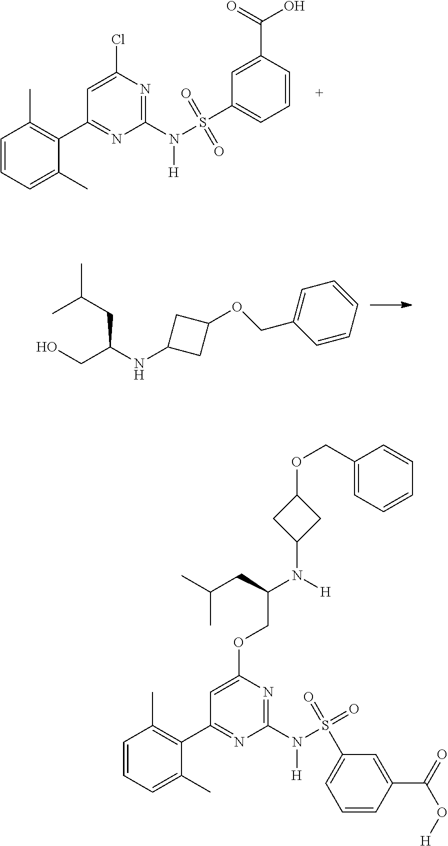 [SMILES: Cc1cccc(C)c1-c1cc(Cl)nc(NS(=O)(=O)c2cccc(C(=O)O)c2)n1 ; CC(C)C[C@H](CO)NC1CC(OCc2ccccc2)C1 ; Cc1cccc(C)c1-c1cc(OC[C@@H](CC(C)C)NC2CC(OCc3ccccc3)C2)nc(NS(=O)(=O)c2cccc(C(=O)O)c2)n1]

In a 250 mL flask, to a stirred solution of 3-[[4-chloro-6-(2,6-dimethylphenyl)pyrimidin-2-yl]sulfamoyl]benzoic acid (5.61 g, 13.43 mmol) in anhydrous tetrahydrofuran (100 mL) was added a solution of (2R)-2-[(3-benzyloxycyclobutyl)amino]-4-methyl-pentan-1-ol (hydrochloride salt) (4.25 g, 13.54 mmol) in anhydrous tetrahydrofuran (10 mL). The heterogeneous mixture was stirred for 5 min while purging nitrogen through it, to form a uniform milky emulsion. To the emulsion, was added sodium tert-butoxide (6.46 g, 67.22 mmol) at once. The reaction was stirred for 1 h at room temperature. The reaction mixture was partitioned between ethyl acetate (150 mL) and an ice-cold hydrochloric acid (82 mL of 1 M, 82.00 mmol) (pH was about 2). The aqueous layer was re-extracted with ethyl acetate (2×50 mL). The combined organics were washed with brine (50 mL), dried over anhydrous sodium sulfate, filtered and concentrated under reduced pressure to obtain crude material 3-[[4-[(2R)-2-[(3-benzyloxycyclobutyl)amino]-4-methyl-pentoxy]-6-(2,6-dimethylphenyl)pyrimidin-2-yl]sulfamoyl]benzoic acid (hydrochloride salt) (9.401 g, 101%) as a white solid. ESI-MS m z calc. 658.28253, found 659.1 (M+1)⁺; Retention time: 1.36 minutes; LC method A.

### Step 3: (11R)-12-(3-benzyloxycyclobutyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 42)

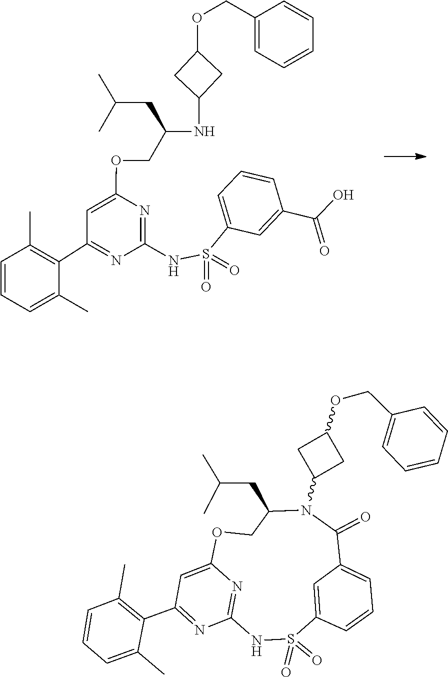 [SMILES: Cc1cccc(C)c1-c1cc(OC[C@@H](CC(C)C)NC2CC(OCc3ccccc3)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC(OCc3ccccc3)C1)[C@H](CC(C)C)CO2]

In a 500 mL flask, to a stirred solution of 3-[[4-[(2R)-2-[(3-benzyloxycyclobutyl)amino]-4-methyl-pentoxy]-6-(2,6-dimethylphenyl)pyrimidin-2-yl]sulfamoyl]benzoic acid (hydrochloride salt) (9.40 g, 13.52 mmol) in anhydrous DMF (175 mL) were added [dimethylamino(triazolo[4,5-b]pyridin-3-yloxy)methylene]-dimethyl-ammonium (Phosphorus Hexafluoride Ion) (5.71 g, 15.02 mmol) (HATU) and DIEA (12.0 mL, 68.89 mmol), in that order, at 0-5° C. (ice-water bath) under nitrogen. The reaction was stirred at that temperature for 30 min, then the bath was removed, and the reaction was allowed to warm to room temperature. After it was stirred overnight (15 h total time) DMF was removed under reduced pressure. The concentrated reaction mixture was poured into a stirred solution of ice-water (150 mL) and hydrochloric acid (80 mL of 1.0 M, 80.00 mmol). The mixture was stirred for 20 min and the resulting pinkish solid was collected by vacuum filtration. The solid was dissolved in ethyl acetate (100 mL) and washed with 1M HCl (100 mL), brine (100 mL), then dried over sodium sulfate and evaporated. The crude material was purified by silica gel (330 g column) chromatography eluting with 0-5% methanol in dichloromethane over 30 min to give as a pink solid (11R)-12-(3-benzyloxycyclobutyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (7.36 g, 85%). Single isomer of unknown syn/anti configuration. ¹H NMR (400 MHz, DMSO-d₆) δ 13.00 (s, 1H), 8.42 (s, 1H), 7.91 (d, J=7.3 Hz, 1H), 7.68 (dtt, J=7.4, 5.1, 2.4 Hz, 2H), 7.36 (d, J=4.2 Hz, 4H), 7.30 (dd, J=5.0, 3.7 Hz, 1H), 7.25 (d, J=7.7 Hz, 1H), 7.12 (d, J=7.7 Hz, 2H), 6.38 (s, 1H), 5.15 (dd, J=10.7, 4.2 Hz, 1H), 4.44 (s, 2H), 4.31 (t, J=11.1 Hz, 1H), 3.81 (dd, J=13.5, 6.5 Hz, 1H), 3.75-3.66 (m, 1H), 3.63-3.51 (m, 1H), 2.96 (q, J=11.1 Hz, 2H), 2.46 (dd, J=7.2, 3.7 Hz, 1H), 2.04-1.91 (m, 6H), 1.70-1.60 (m, 1H), 1.29 (ddd, J=9.3, 6.6, 3.2 Hz, 1H), 1.18-1.13 (m, 1H), 0.89 (dq, J=11.5, 6.1, 5.3 Hz, 1H), 0.74 (d, J=6.6 Hz, 3H), 0.22 (d, J=6.4 Hz, 3H). ESI-MS m/z calc. 640.2719, found 641.1 (M+1)⁺; Retention time: 2.08 minutes LC method A.

### Example 37: Preparation of Compound 43 and Compound 44

### Step 1: (11R)-6-(2,6-dimethylphenyl)-12-(3-hydroxycyclobutyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound 43), and (11R)-6-(2,6-dimethylphenyl)-12-(3-hydroxycyclobutyl)-11-isobutyl-2,2-dioxo-9-oxa-2%⁶-thia-3,5,12,19-tetrazatricyclo[12.3.1.14,8]nonadeca-1(18),4(19),5,7,14,16-hexaen-13-one, diastereomer 2 (Compound 44)

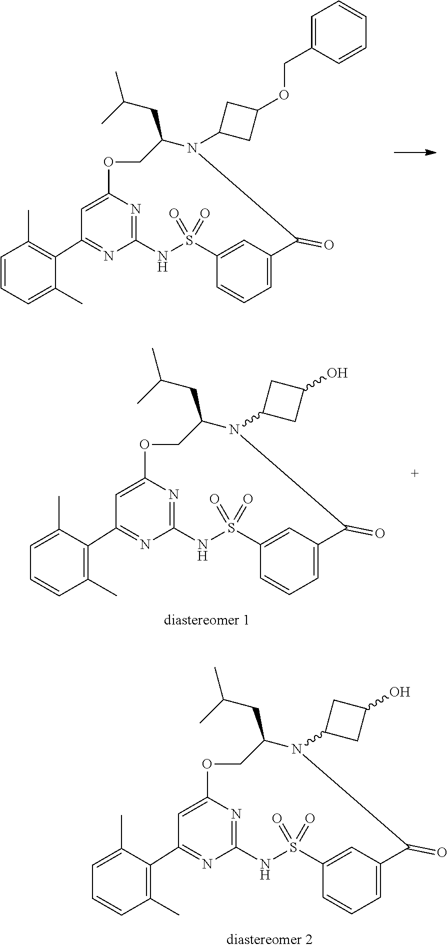 [SMILES: Cc1cccc(C)c1-c1cc2nc(n1)NS(=O)(=O)c1cccc(c1)C(=O)N(C1CC(OCc3ccccc3)C1)[C@H](CC(C)C)CO2 ; Cc1cccc(C)c1-c1cc2nc(n1)NS(=O)(=O)c1cccc(c1)C(=O)N(C1CC(O)C1)[C@H](CC(C)C)CO2 ; Cc1cccc(C)c1-c1cc2nc(n1)NS(=O)(=O)c1cccc(c1)C(=O)N(C1CC(O)C1)[C@H](CC(C)C)CO2]

(11R)-12-(3-Benzyloxycyclobutyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (5 mg, 0.007803 mmol) was dissolved in methanol (1 mL), dihydroxypalladium (2 mg, 0.002848 mmol) was added, and the reaction vessel was purged with nitrogen. Hydrogen gas was bubbled through the reaction mixture from a balloon for 1 hour. The reaction mixture was then purged with nitrogen, filtered, and purified by reverse phase HPLC (1-99% ACN with HCl modifier, 30 min run) to give separately two presumed relative isomers of the cyclobutane (syn and anti not known), (11R)-6-(2,6-dimethylphenyl)-12-(3-hydroxycyclobutyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (1.2 mg, 28%) diastereomer 1 ESI-MS m/z calc. 550.225, found 551.5 (M+1)⁺; Retention time: 1.44 minutes (Peak 1), LC method A; and (11R)-6-(2,6-dimethylphenyl)-12-(3-hydroxycyclobutyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (2.2 mg, 51%) diastereomer 2, ESI-MS m/z calc. 550.225, found 551.4 (M+1)⁺; Retention time: 1.51 minutes (Peak 2); LC method A.

### Example 38: Preparation of Compound 45, Compound 46, and Compound 47

### Step 1: 3-[[4-(2,6-Dimethylphenyl)-6-[(2R)-2-[(3-isopropoxycyclobutyl)amino]-4-methyl-pentoxy]pyrimidin-2-yl]sulfamoyl]benzoic acid

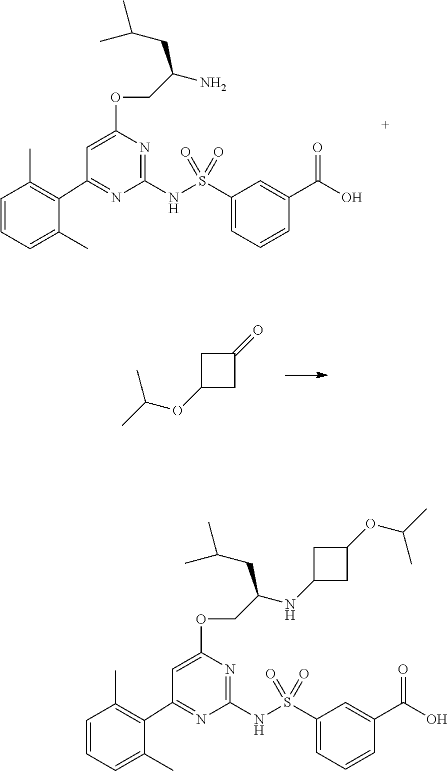

3-[[4-[(2R)-2-amino-4-methyl-pentoxy]-6-(2,6-dimethylphenyl)pyrimidin-2-yl]sulfamoyl]benzoic acid (hydrochloride salt) (98.6 mg, 0.1843 mmol), 3-isopropoxycyclobutanone (31.5 mg, 0.2458 mmol), and sodium triacetoxyborohydride (104.5 mg, 0.5531 mmol) were combined in DCM (0.3 mL) and stirred at room temperature for 5 h. The reaction was diluted with methanol (0.7 mL) and DMSO (2 mL), filtered and purified by LC/MS utilizing a gradient of 1-99% acetonitrile in 5 mM aqueous HCl to yield 3-[[4-(2,6-dimethylphenyl)-6-[(2R)-2-[(3-isopropoxycyclobutyl)amino]-4-methyl-pentoxy]pyrimidin-2-yl]sulfamoyl]benzoic acid (hydrochloride salt) (38.3 mg, 32%) ESI-MS m/z calc. 610.28253, found 611.4 (M+1)⁺; Retention time: 0.51 minutes (LC method A).

### Step 2: (11R)-6-(2,6-Dimethylphenyl)-11-isobutyl-12-(3-isopropoxycyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 47)

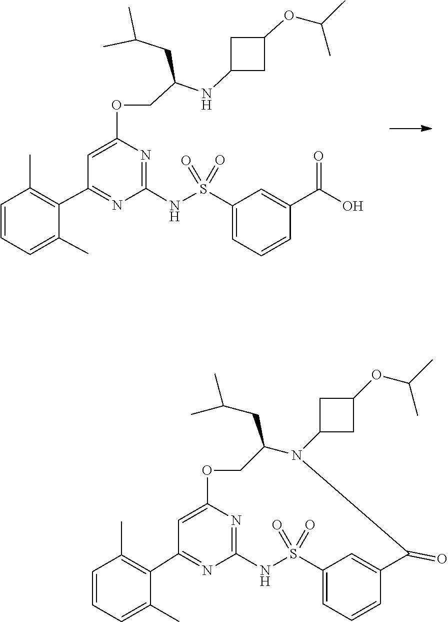 [SMILES: Cc1cccc(C)c1-c1cc(OC[C@@H](CC(C)C)NC2CC(OC(C)C)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC(OC(C)C)C1)[C@H](CC(C)C)CO2]

3-[[4-(2,6-Dimethylphenyl)-6-[(2R)-2-[(3-isopropoxycyclobutyl)amino]-4-methyl-pentoxy]pyrimidin-2-yl]sulfamoyl]benzoic acid (hydrochloride salt) (38.3 mg, 0.05918 mmol), HATU (27 mg, 0.07101 mmol), and triethylamine (29.68 μL, 0.2129 mmol) were combined in DMF (1 mL) and stirred at room temperature for 2 h. The reaction was filtered and purified by LC/MS utilizing a gradient of 1-99% acetonitrile in 5 mM aqueous HCl to yield (11R)-6-(2,6-dimethylphenyl)-11-isobutyl-12-(3-isopropoxycyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one (22.2 mg, 53%) ESI-MS m/z calc. 592.2719, found 593.2 (M+1)⁺; Retention time: 1.98 minutes (LC method A).

### Step 3: (11R)-6-(2,6-Dimethylphenyl)-11-isobutyl-12-(3-isopropoxycyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound 45), and (11R)-6-(2,6-dimethylphenyl)-11-isobutyl-12-(3-isopropoxycyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 2 (Compound 46)

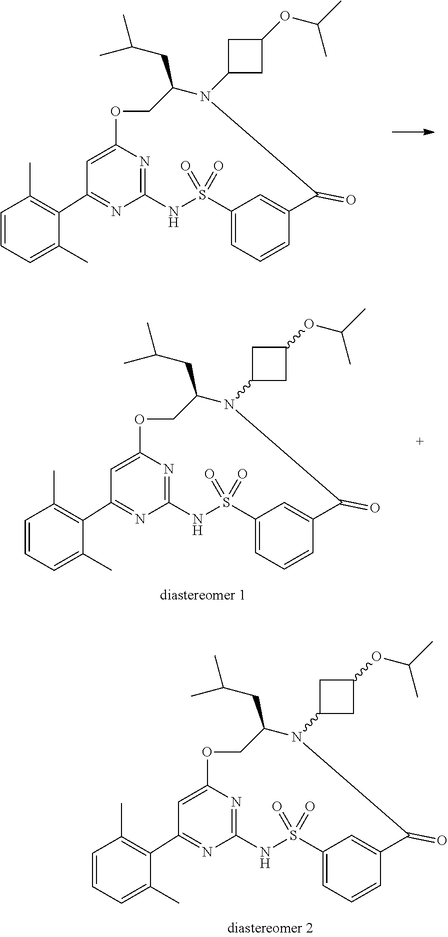 [SMILES: Cc1cccc(C)c1-c1cc2nc(n1)NS(=O)(=O)c1cccc(c1)C(=O)N(C1CC(OC(C)C)C1)[C@H](CC(C)C)CO2 ; Cc1cccc(C)c1-c1cc2nc(n1)NS(=O)(=O)c1cccc(c1)C(=O)N(C1CC(OC(C)C)C1)[C@H](CC(C)C)CO2 ; Cc1cccc(C)c1-c1cc2nc(n1)NS(=O)(=O)c1cccc(c1)C(=O)N(C1CC(OC(C)C)C1)[C@H](CC(C)C)CO2]

(11R)-6-(2,6-Dimethylphenyl)-11-isobutyl-12-(3-isopropoxycyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one (20 mg, 0.03374 mmol) was submitted for SFC separation using a Chiral Pak AS-H (250×21.2 mm), 5 m column at 40° C., mobile phase:14% MeOH (no modifier), 86% CO₂, flow rate 70 mL/min, concentration 24 mg/mL, injection volume 500 μL, 158 bar, 210 nm. Two isomers were isolated: Peak 1, diastereomer 1, (11R)-6-(2,6-dimethylphenyl)-11-isobutyl-12-(3-isopropoxycyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one (2.9 mg, 15%) ESI-MS m/z calc. 592.2719, found 593.3 (M+1)⁺; Retention time: 2.07 minutes (LC method A), and peak 2, diastereomer 2, (11R)-6-(2,6-dimethylphenyl)-11-isobutyl-12-(3-isopropoxycyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one (14.8 mg, 74%) ESI-MS m/z calc. 592.2719, found 593.3 (M+1)⁺; Retention time: 2.06 minutes (LC method A).

### Example 39: Preparation of Compound 48

### Step 1: (11R)-6-(2,6-Dimethylphenyl)-12-(3-hydroxycyclobutyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one

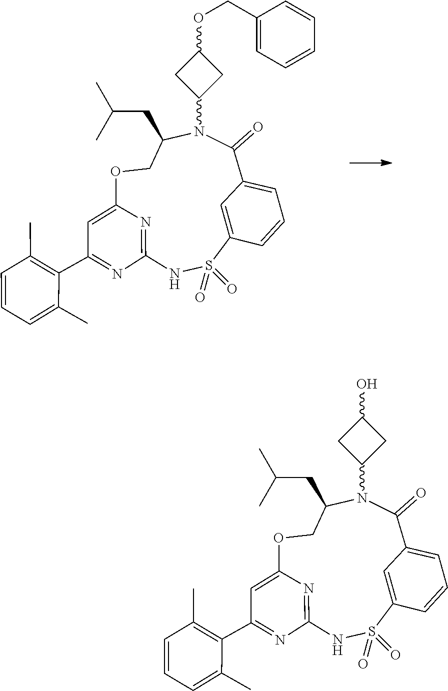 [SMILES: Cc1cccc(C)c1-c1cc2nc(n1)NS(=O)(=O)c1cccc(c1)C(=O)N(C1CC(OCc3ccccc3)C1)[C@H](CC(C)C)CO2 ; Cc1cccc(C)c1-c1cc2nc(n1)NS(=O)(=O)c1cccc(c1)C(=O)N(C1CC(O)C1)[C@H](CC(C)C)CO2]

In a 250 mL 3-necked flask, a stirred solution of (11R)-12-(3-benzyloxycyclobutyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (7.0 g, 10.92 mmol) in anhydrous methanol (100 mL) was purged with nitrogen for 10 min. Carefully palladium hydroxide (1.50 g of 20% w/w, 2.136 mmol) was added and the reaction vessel was evacuated and replenished with nitrogen again (twice). Then hydrogen gas-filled balloon was connected and stirring continued at ambient temperature for 5 h. More palladium hydroxide (1.50 g of 20% w/w, 2.136 mmol) was added under nitrogen and the above purging was repeated. After 8 h, more palladium hydroxide (1.50 g of 20% w/w, 2.136 mmol) and stirring was continued overnight (total 24 h). The flask was evacuated and replenished with nitrogen and water (5 mL was added and the reaction was stirred for 10 min, and the dark reaction mixture was filtered over a pad of Celite and the filter cake was further washed with methanol. The filtrates were concentrated under reduced pressure to give (11R)-6-(2,6-dimethylphenyl)-12-(3-hydroxycyclobutyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (5.87 g, 98%) off-white color. ESI-MS m/z calc. 550.225, found 551.0 (M+1)⁺; Retention time: 1.53 minutes LC method A.

### Step 2: [3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl] N,N-dimethylcarbamate (Compound 48)

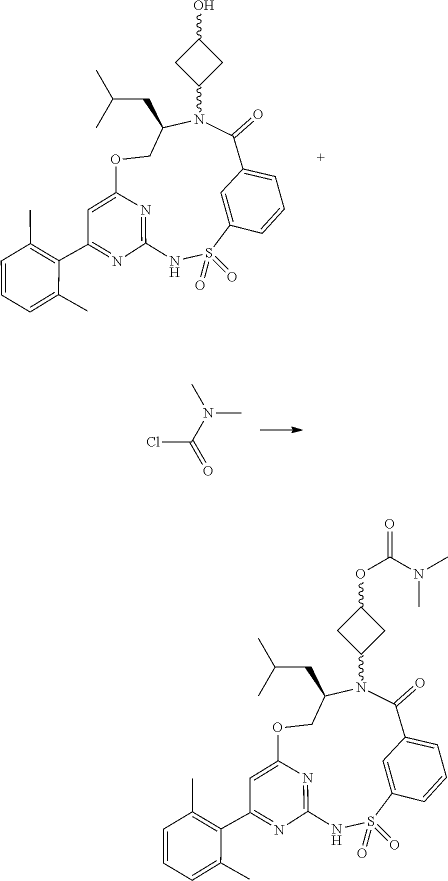

(11R)-6-(2,6-Dimethylphenyl)-12-(3-hydroxycyclobutyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (10 mg, 0.01816 mmol) was combined with NaH (6 mg, 0.1500 mmol) in DMF (0.5 mL) and N,N-dimethylcarbamoyl chloride (8 mg, 0.07439 mmol) was added, and the reaction was stirred for 2 hours at room temperature. The reaction mixture was then quenched with several drops of 1M HCl, diluted with methanol, filtered and purified by reverse phase HPLC (1-99% ACN in water, HCl modifier, 15 min run). A small amount of the minor cyclobutyl stereoisomer overlapped, and the product was re-purified by reverse phase HPLC (1-70% ACN in water, HCl modifier, 15 min run) to give [3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl] N,N-dimethylcarbamate (4.2 mg, 37%) as a white powder ESI-MS m/z calc. 621.2621, found 622.7 (M+1)⁺; Retention time: 1.73 minutes (LC method A).

### Example 40: Preparation of Compound 49 and Compound 50

### Step 1: 3-Benzyloxy-1-methyl-cyclobutanol

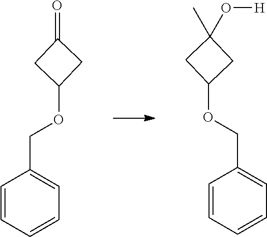

3-Benzyloxycyclobutanone (503 mg, 2.8545 mmol) was dissolved in diethylether (1.4 mL) then methyl magnesium bromide 3M in diethylether (1.40 mL of 3 M, 4.2000 mmol) was added drop wise at room temperature. The reaction was stirred for an hour then cooled to 0° C. and quenched with ammonium chloride (5 mL). The mixture was diluted with EtOAc (5 mL) and the layers separated. The aqueous layer was extracted with EtOAc 2 more times (2×5 mL), dried over sodium sulfate and concentrated. The residue was dry loaded on to silica gel and purified by flash column chromatography using 0-30% EtOAc in Hexanes to give 3-benzyloxy-1-methyl-cyclobutanol (283 mg, 46%) as a colorless oil and as 1:1 mixture of isomers. ¹H NMR (250 MHz, DMSO-d₆) δ 7.40-7.24 (m, 5H), 4.40-4.29 (m, 2H), 2.33-2.12 (m, 2H), 2.02-1.84 (m, 2H), 1.15 and 1.28 (two s, 3H total).

### Step 2: [3-Methyl-3-(trifluoromethoxy)cyclobutoxy]methylbenzene

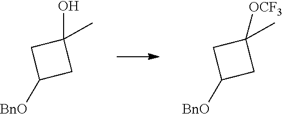 [SMILES: CC1(O)CC(OCc2ccccc2)C1 ; CC1(OC(F)(F)F)CC(OCc2ccccc2)C1]

3-Benzyloxy-1-methyl-cyclobutanol (9.23 g, 48.009 mmol) was dissolved in ethyl acetate (325 mL) then silver triflate (37.05 g, 144.20 mmol), Selectfluor (25.61 g, 72.292 mmol) and potassium fluoride (11.02 g, 189.68 mmol) were added. The vessel was flushed with nitrogen and 2-fluoropyridine (14.100 g, 12.5 mL, 145.23 mmol) and trifluoromethyltrimethylsilane (20.683 g, 21.5 mL, 145.46 mmol) were added. The mixture was allowed to stir for 3 days at room temperature under a nitrogen atmosphere. The mixture was filtered through a pad of Celite, and dry loaded on to silica gel and purified by flash column chromatography using 0-30% ethyl acetate in hexanes. The appropriate fractions were collected to give [3-methyl-3-(trifluoromethoxy)cyclobutoxy]methylbenzene (2.58 g, 19%) as a colorless oil. ¹H NMR (250 MHz, CDCl₃) δ 7.47-7.16 (m, 5H), 4.43 (s, 2H), 3.77 (p, J=6.9 Hz, 1H), 2.49 (d, J=6.3 Hz, 4H), 1.50 (s, 3H). Note: Peak at 5.30 is DCM

### Step 3: 3-Methyl-3-(trifluoromethoxy)cyclobutanol

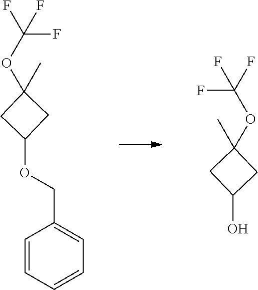 [SMILES: CC1(OC(F)(F)F)CC(OCc2ccccc2)C1 ; CC1(OC(F)(F)F)CC(O)C1]

[3-Methyl-3-(trifluoromethoxy)cyclobutoxy]methylbenzene (635 mg, 2.4399 mmol) was dissolved in methyl acetate (15.875 mL) and Pd/C (683 mg, 10% w/w, 0.6418 mmol) was added. The reaction was placed under a hydrogen atmosphere (balloon) and allowed to stir 48 h. Celite was added and the solids filtered off and rinsed with diethylether. The filtrate was concentrated to give 3-methyl-3-(trifluoromethoxy)cyclobutanol (364.5 mg, 79%) as a colorless oil. ¹H NMR (250 MHz, CDCl₃) δ 4.04 (p, J=7.0 Hz, 1H), 2.67-2.49 (m, 2H), 2.49-2.32 (m, 2H), 1.87 (bs, 1H), 1.55-1.42 (m, 3H).

### Step 4: (2R)-4-Methyl-2-[[3-methyl-3-(trifluoromethoxy)cyclobutyl]amino]pentan-1-ol

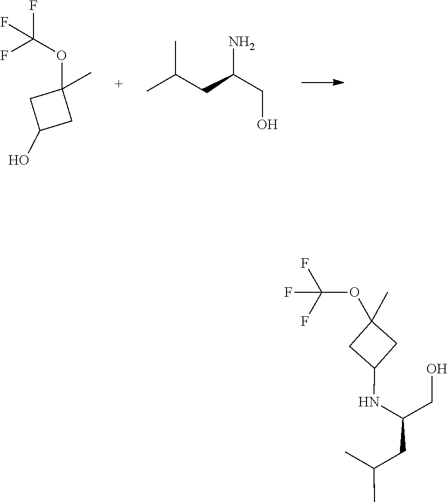

Into a solution of 3-methyl-3-(trifluoromethoxy)cyclobutanol (100 mg, 0.5878 mmol) and pyridine (119 mg, 1.5044 mmol) in anhydrous DCM (1 mL) was added trifluoromethylsulfonyl trifluoromethanesulfonate (270 mg, 0.9570 mmol) at 0° C. The reaction was stirred at 25° C. for 2 hours. The reaction was diluted with hexane (5 mL), and the solution was washed with 10% HCl (2 mL), saturated sodium bicarbonate (2 mL) and brine (2 mL). The solution was dried over anhydrous sodium sulfate and concentrated under vacuum at room temperature bath to furnish a crude triflate. This product and (2R)-2-amino-4-methyl-pentan-1-ol (93 mg, 0.7936 mmol) were dissolved in MeCN (1 mL). 4 Å molecular sieves (50 mg) and potassium carbonate (398 mg, 2.8798 mmol) were added to the reaction mixture. The reaction was stirred at room temperature for 16 hours. The reaction was heated to 80° C. for 1 h and then the molecular sieves were filtered off through a pad of Celite. The filtrate was concentrated under vacuum. The residue was purified by silica gel chromatography using ethyl acetate to furnish (2R)-4-methyl-2-[[3-methyl-3-(trifluoromethoxy)cyclobutyl]amino]pentan-1-ol (113.5 mg, 65%) as a light yellow solid (mixture of diastereomers), ESI-MS m/z calc. 269.1603, found 270.5 (M+1)⁺; Retention time: 2.19 minutes; LC method T.

### Step 5: 3-[[4-(2,6-Dimethylphenyl)-6-[(2R)-4-methyl-2-[[3-methyl-3-(trifluoromethoxy)cyclobutyl]amino]pentoxy]pyrimidin-2-yl]sulfamoyl]benzoic acid

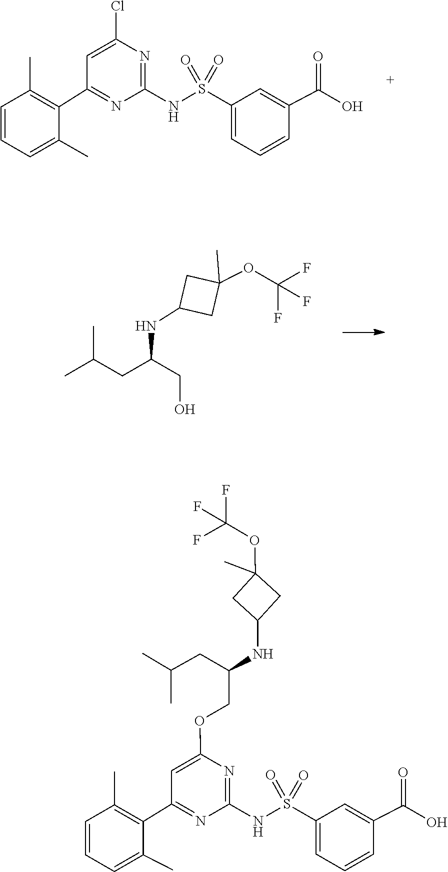 [SMILES: Cc1cccc(C)c1-c1cc(Cl)nc(NS(=O)(=O)c2cccc(C(=O)O)c2)n1 ; CC(C)C[C@H](CO)NC1CC(C)(OC(F)(F)F)C1 ; Cc1cccc(C)c1-c1cc(OC[C@@H](CC(C)C)NC2CC(C)(OC(F)(F)F)C2)nc(NS(=O)(=O)c2cccc(C(=O)O)c2)n1]

(2R)-4-Methyl-2-[[3-methyl-3-(trifluoromethoxy)cyclobutyl]amino]pentan-1-ol (171 mg, 0.6699 mmol) and 3-[[4-chloro-6-(2,6-dimethylphenyl)pyrimidin-2-yl]sulfamoyl]benzoic acid (313 mg, 0.7490 mmol) were dissolved in THE (9 mL) then sodium tert-butoxide (654 mg, 6.8052 mmol) was added and the reaction stirred at room temperature for 1 hour. The reaction was quenched with 2M HCl (12 mL), then extracted with CHCl3 three times (3×10 mL). The organic layers were washed with brine (12 mL), then dried over sodium sulfate and concentrated. The crude residue was combined with a crude from another reaction and purified using 0-10% MeOH in DCM to give 3-[[4-(2,6-dimethylphenyl)-6-[(2R)-4-methyl-2-[[3-methyl-3-(trifluoromethoxy)cyclobutyl]amino]pentoxy]pyrimidin-2-yl]sulfamoyl]benzoic acid (hydrochloride salt) (337.8 mg, 66% corrected yield) as a white solid. ESI-MS m/z calc. 650.2386, found 651.6 (M+1)⁺; Retention time: 2.77 minutes; LC method T.

### Step 6: (11R)-6-(2,6-dimethylphenyl)-11-isobutyl-12-[3-methyl-3-(trifluoromethoxy)cyclobutyl]-2,2-dioxo-9-oxa-2λ⁶-thia-3,5,12,19-tetrazatricyclo[12.3.1.14,8]nonadeca-1(18),4(19),5,7,14,16-hexaen-13-one, major isomer (Compound 49), and (11R)-6-(2,6-dimethylphenyl)-11-isobutyl-12-[3-methyl-3-(trifluoromethoxy)cyclobutyl]-2,2-dioxo-9-oxa-2λ⁶-thia-3,5,12,19-tetrazatricyclo[12.3.1.14,8]nonadeca-1(18),4(19),5,7,14,16-hexaen-13-one, minor isomer (Compound 50)

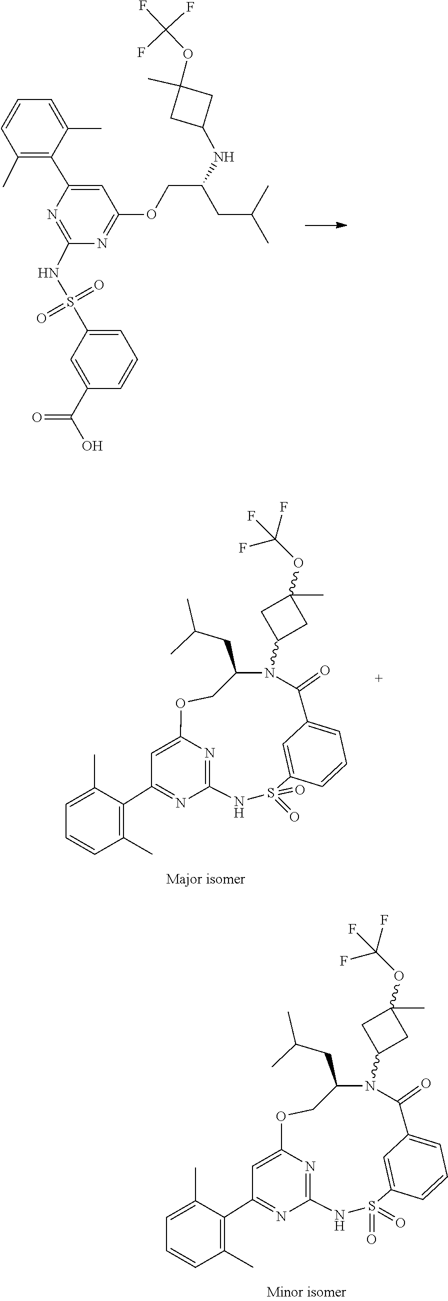

3-[[4-(2,6-dimethylphenyl)-6-[(2R)-4-methyl-2-[[3-methyl-3-(trifluoromethoxy)cyclobutyl]amino]pentoxy]pyrimidin-2-yl]sulfamoyl]benzoic acid (417.8 mg, 0.6421 mmol) was dissolved in DMF (7 mL) and DIPEA (408.10 mg, 0.55 mL, 3.1576 mmol) was added. Then a solution of HATU (349 mg, 0.9179 mmol) in DMF (7 mL) was added drop wise at room temperature. The reaction was stirred at room temperature overnight, and then quenched by the addition of brine (60 mL). The aqueous layer was extracted three times with EtOAc (3×20 mL). The organic layer was washed 4 times with brine (4×10 mL), dried over sodium sulfate and concentrated. The crude residue was dry loaded on to silica gel and purified by flash column chromatography using 0-10% MeOH in DCM. The appropriate fractions were collected and submitted for purification by reverse phase HPLC using 0 to 100% acetonitrile in water (buffered with 0.1% TFA) to furnish to give two diastereomers of (11R)-6-(2,6-dimethylphenyl)-11-isobutyl-12-[3-methyl-3-(trifluoromethoxy)cyclobutyl]-2,2-dioxo-9-oxa-2λ⁶-thia-3,5,12,19-tetrazatricyclo[12.3.1.14,8]nonadeca-1(18),4(19),5,7,14,16-hexaen-13-one (160 mg, 37%) and (11R)-6-(2,6-dimethylphenyl)-11-isobutyl-12-[3-methyl-3-(trifluoromethoxy)cyclobutyl]-2,2-dioxo-9-oxa-2λ⁶-thia-3,5,12,19-tetrazatricyclo[12.3.1.14,8]nonadeca-1(18),4(19),5,7,14,16-hexaen-13-one as tan solids.

Minor isomer (11.4 mg, 3%): ESI-MS m/z calc. 632.228, found 633.5 (M+1)⁺; Retention time: 3.02 minutes; LC method W. ¹H NMR (500 MHz, DMSO-d₆) δ 8.45 (s, 1H), 7.92 (d, J=7.6 Hz, 1H), 7.80-7.60 (m, 2H), 7.27 (t, J=7.6 Hz, 1H), 7.13 (d, J=7.6 Hz, 2H), 6.40 (s, 1H), 5.15 (dd, J=10.7, 4.3 Hz, 1H), 4.41 (t, J=11.1 Hz, 1H), 3.92-3.64 (m, 3H), 3.64-3.53 (m, 2H), 2.43-2.26 (m, 2H), 1.98 (s, 6H), 1.66 (s, 3H), 1.32-1.21 (m, 1H), 1.16 (t, J=13.5 Hz, 1H), 0.78-0.68 (m, 3H), 0.21 (d, J=6.3 Hz, 3H)

Major Isomer (160 mg, 37%): ESI-MS m/z calc. 632.228, found 633.5 (M+1)⁺; Retention time: 3.16 minutes; LC method W. ¹H NMR (500 MHz, DMSO-d₆) δ 8.44 (s, 1H), 7.92 (d, J=7.6 Hz, 1H), 7.78-7.61 (m, 2H), 7.27 (t, J=7.6 Hz, 1H), 7.13 (d, J=7.6 Hz, 2H), 6.40 (s, 1H), 5.14 (dd, J=10.7, 4.3 Hz, 1H), 4.40 (t, J=11.1 Hz, 1H), 4.25 (p, J=8.9 Hz, 1H), 3.74 (td, J=11.1, 9.4, 5.5 Hz, 2H), 3.20 (ddd, J=11.7, 8.5, 2.6 Hz, 2H), 2.65 (dddd, J=42.7, 13.0, 9.0, 4.1 Hz, 2H), 1.99 (s, 6H), 1.74 (d, J=1.8 Hz, 3H), 1.60 (ddd, J=13.8, 10.7, 2.7 Hz, 1H), 1.32-1.12 (m, 2H), 0.73 (d, J=6.5 Hz, 3H), 0.21 (d, J=6.3 Hz, 3H).

### Example 41: Preparation of Compound 51

### Step 1: Benzyl 2-[(4R)-2-oxooxazolidin-4-yl]acetate

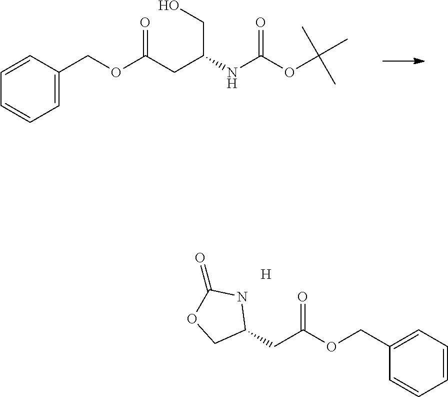

To a solution of benzyl (3R)-3-(tert-butoxycarbonylamino)-4-hydroxy-butanoate (27.8 g, 89.864 mmol)benzyl (3R)-3-(tert-butoxycarbonylamino)-4-hydroxy-butanoate (27.8 g, 89.864 mmol) in 1,2-dichloroethane (250 mL) was added pyridine (65.526 g, 67 mL, 828.40 mmol) and the mixture was cooled to 0-5° C. p-toluenesulfonic anhydride (32.263 g, 98.850 mmol) was added and the mixture was warmed to room temperature and stirred for 2 hours and then heated to 90° C. for 2 hours. The mixture was cooled, diluted with dichloromethane (500 mL) and washed with 1N HCl (3×200 mL). The combined aqueous layers were back extracted with dichloromethane (2×150 mL). The combined organic layers were dried with sodium sulfate, filtered and concentrated to dryness. The crude material was purified by flash chromatography (330 g) using a gradient of 20% to 100% ethyl acetate in heptane to afford enantiopure benzyl 2-[(4R)-2-oxooxazolidin-4-yl]acetate (18.11 g, 86%) as a white solid. ¹H NMR (400 MHz, CDCl₃) δ 7.44-7.31 (m, 5H), 5.58 (br. s., 1H), 5.16 (s, 2H), 4.56 (t, J=8.6 Hz, 1H), 4.25 (qd, J=7.0, 5.9 Hz, 1H), 4.06 (dd, J=8.9, 5.7 Hz, 1H), 2.76-2.63 (m, 2H). ESI-MS m/z calc. 235.0845, found 236.2 (M+1)⁺, 471.2 (2M+H)⁺; Retention time: 1.49 minutes; LC method X.

### Step 2: (4R)-4-(2-Hydroxy-2-methyl-propyl)oxazolidin-2-one

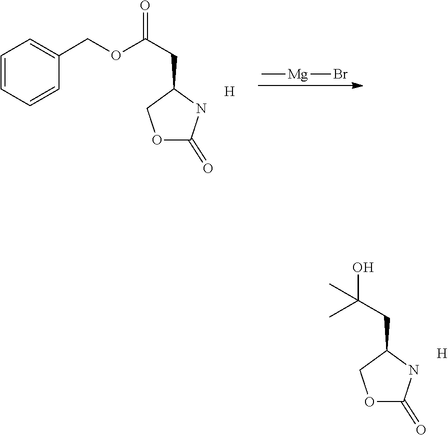

Bromo(methyl)magnesium in diethyl ether (105 mL of 3 M, 315.00 mmol) was added to a mixture of toluene (150 mL) and THE (150 mL) at −20 oC. A warm THE (80 mL) solution of benzyl 2-[(4R)-2-oxooxazolidin-4-yl]acetate (18.1 g, 76.944 mmol) was then added dropwise maintaining the temperature below −10 oC. The mixture was warm up to room temperature and stirred for 18 hours. The mixture was added via canula to a solution of acetic acid (85 mL) in water (440 mL) at 0° C. The resultant mixture was stirred for 1 hour at room temperature. The layers were separated. The aqueous layer was saturated with brine (200 mL) and further extracted with 2-methyltetrahydrofuran (3×250 mL) and with ethanol/chloroform (½, 3×330 mL). The combined organic extracts were dried over anhydrous sodium sulfate, filtered and concentrated. The residue was co-evaporated with heptanes (4×100 mL). The crude material was purified in two equal batches by flash chromatography (330 g) eluting with 6% isopropanol in dichloromethane) to give (4R)-4-(2-hydroxy-2-methyl-propyl)oxazolidin-2-one (8.88 g, 69%) as an off-white solid. ¹H NMR (400 MHz, DMSO-d₆) δ 7.36 (s, 1H), 4.45-4.38 (m, 1H), 4.36 (s, 1H), 4.00-3.91 (m, 2H), 1.68-1.54 (m, 2H), 1.10 (s, 6H). ESI-MS m/z calc. 159.0895, found 160.2 (M+1)⁺; Retention time: 0.77 minutes, LC method X.

### Step 3: (2R)-2-Amino-4-methyl-pentane-1,4-diol

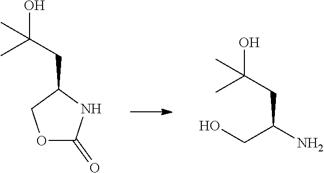

A mixture of (4R)-4-(2-hydroxy-2-methyl-propyl)oxazolidin-2-one (904 mg, 4.2592 mmol) and barium hydroxide octahydrate (4.03 g, 12.775 mmol) in ethanol (20 mL) and water (20 mL) was stirred at 90-95° C. for 4 hours. After cooling down to room temperature, dry ice (˜7 g) was added and the mixture was stirred vigorously for 2 days. The suspension was filtered over a Celite pad and rinsed with ethanol (20 mL). The filtrate was diluted with toluene and concentrated under reduced pressure to provide (2R)-2-amino-4-methyl-pentane-1,4-diol (780 mg) which was used without further purification for the next step. ¹H NMR (400 MHz, DMSO-d₆) δ 5.12 (br. s., 2H), 3.30-3.16 (m, 2H), 2.94 (dd, J=9.0, 3.4 Hz, 1H), 1.83 (s, 2H), 1.49-1.40 (m, 1H), 1.33-1.21 (m, 1H), 1.11 (d, J=11.0 Hz, 6H). ESI-MS m/z calc. 133.1103, found 134.4 (M+1)⁺; Retention time: 0.21 minutes, LC method X.

### Step 4: 3-[[4-[(2R)-2-Amino-4-hydroxy-4-methyl-pentoxy]-6-(2,6-dimethylphenyl)pyrimidin-2-yl]sulfamoyl]benzoic acid

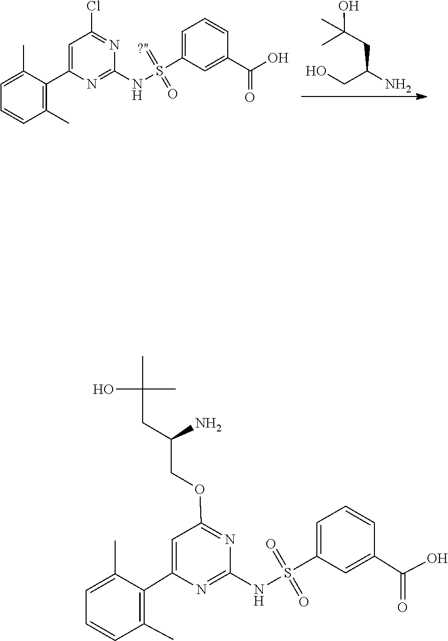

To a solution of (2R)-2-amino-4-methyl-pentane-1,4-diol (567 mg, 4.2571 mmol) and 3-[[4-chloro-6-(2,6-dimethylphenyl)pyrimidin-2-yl]sulfamoyl]benzoic acid (1.5 g, 3.5897 mmol) in tetrahydrofuran (6 mL) was slowly added sodium tert-butoxide in tetrahydrofuran (7.2 mL of 2 M, 14.400 mmol) and the mixture was stirred at room temperature for one hour. The reaction was partitioned between ethyl acetate (30 mL) and 1 N hydrochloric acid (30 mL). The aqueous phase was extracted with ethyl acetate (2×20 mL) and 2-methyltetrahydrofuran (4×30 mL). The combined organic layers were dried over sodium sulfate, filtered and concentrated to dryness. The residue was triturated with ethyl acetate (20 mL), the precipitate was filtered and washed with ethyl acetate (2×10 mL). The product was further dried under vacuum to afford 3-[[4-[(2R)-2-amino-4-hydroxy-4-methyl-pentoxy]-6-(2,6-dimethylphenyl)pyrimidin-2-yl]sulfamoyl]benzoic acid (hydrochloride salt) (1.62 g, 80%) as a pale-yellow solid. ¹H NMR (400 MHz, DMSO-d₆) δ 13.07 (br. s., 2H), 8.43 (s, 1H), 8.14 (d, J=7.8 Hz, 2H), 8.10-8.01 (m, 3H), 7.70 (t, J=7.7 Hz, 1H), 7.32-7.22 (m, 1H), 7.13 (d, J=7.6 Hz, 2H), 6.29 (br. s., 1H), 5.13 (br. s., 1H), 4.36 (dd, J=11.5, 2.9 Hz, 1H), 4.18 (dd, J=11.4, 7.7 Hz, 1H), 3.83-3.70 (m, 1H), 2.02 (s, 6H), 1.71 (d, J=6.4 Hz, 2H), 1.24 (m, 6H). ESI-MS m/z calc. 514.1886, found 515.2 (M+1)⁺; Retention time: 1.3 minutes, LC method X.

### Step 5: 3-[[4-(2,6-Dimethylphenyl)-6-[(2R)-4-hydroxy-4-methyl-2-(spiro[2.3]hexan-5-ylamino)pentoxy]pyrimidin-2-yl]sulfamoyl]benzoic acid

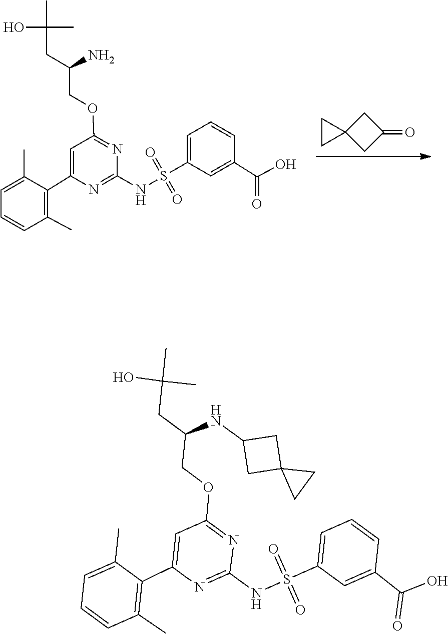 [SMILES: Cc1cccc(C)c1-c1cc(OC[C@H](N)CC(C)(C)O)nc(NS(=O)(=O)c2cccc(C(=O)O)c2)n1 ; O=C1CC2(CC2)C1 ; Cc1cccc(C)c1-c1cc(OC[C@@H](CC(C)(C)O)NC2CC3(CC3)C2)nc(NS(=O)(=O)c2cccc(C(=O)O)c2)n1]

3-[[4-[(2R)-2-amino-4-hydroxy-4-methyl-pentoxy]-6-(2,6-dimethylphenyl)pyrimidin-2-yl]sulfamoyl]benzoic acid (50 mg, 0.0972 mmol), spiro[2.3]hexan-5-one (16 mg, 0.1664 mmol) and acetic acid (2 mg, 0.0019 mL, 0.0333 mmol) were stirred in acetonitrile (1 mL) and methanol (0.7 mL, to solubilize starting material) for 30 min. and then sodium cyanoborohydride (22 mg, 0.3501 mmol) was added to the solution which was then stirred at room temperature overnight. More spiro[2.3]hexan-5-one (10 mg, 0.1040 mmol) was added stirred 1 hour and then sodium cyanoborohydride (22 mg, 0.3501 mmol) was added and the reaction mixture was left stirring for 2 h. More spiro[2.3]hexan-5-one (10 mg, 0.1040 mmol) was added and then left stirring for 1 h then sodium cyanoborohydride (110 mg, 1.7504 mmol) was added and the reaction mixture was left stirring at room temperature overnight. The reaction mixture was partitioned between ethyl acetate (50 mL) and saturated aqueous ammonium chloride (20 mL). The aqueous phase was separated and washed with ethyl acetate (20 mL). The organic phases were combined, washed with brine (10 mL), dried with sodium sulfate, filtered and concentrated under reduced pressure to provide 3-[[4-(2,6-dimethylphenyl)-6-[(2R)-4-hydroxy-4-methyl-2-(spiro[2.3]hexan-5-ylamino)pentoxy]pyrimidin-2-yl]sulfamoyl]benzoic acid (66 mg, 114%) as a clear oil ESI-MS m/z calc. 594.2512, found 595.3 (M+1)⁺; Retention time: 1.41 minutes which was used in the next step without further purification. LC method X.

### Step 6: (11R)-6-(2,6-Dimethylphenyl)-11-(2-hydroxy-2-methyl-propyl)-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (Compound 51)

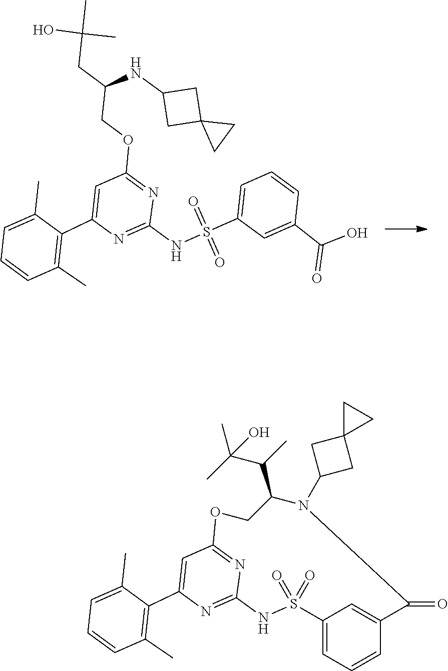 [SMILES: Cc1cccc(C)c1-c1cc(OC[C@@H](CC(C)(C)O)NC2CC3(CC3)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CC3)C1)[C@H](C(C)C(C)(C)O)CO2]

To a solution of 3-[[4-(2,6-dimethylphenyl)-6-[(2R)-4-hydroxy-4-methyl-2-(spiro[2.3]hexan-5-ylamino)pentoxy]pyrimidin-2-yl]sulfamoyl]benzoic acid (110 mg, 0.1850 mmol) and triethyl amine (72.600 mg, 0.1 mL, 0.7175 mmol) in DMF (1.5 mL) and ethyl acetate (5.5 mL) stirred at 0° C. was added T3P (50% in ethyl acetate) (106.90 mg, 200 μL, 0.1680 mmol) dropwise and the reaction mixture was warmed-up to room temperature for 1 h. More triethyl amine (145.20 mg, 0.2 mL, 1.4349 mmol) and then T3P (50% in ethyl acetate) (267.25 mg, 0.5 mL, 0.4200 mmol) were added and the reaction mixture was stirred for 3 h at room temperature. The reaction mixture was concentrated under reduced pressure and the resulting crude was left on high vacuum pump for 1 h then injected directly on column and purified by reverse phase chromatography using 0.5 to 100% acetonitrile in water. The pure fractions were combined and concentrated on the freeze drier to provide (11R)-6-(2,6-dimethylphenyl)-11-(2-hydroxy-2-methyl-propyl)-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (18 mg, 17%) as a white fluffy solid ¹H NMR (taken at 80° C.) (400 MHz, DMSO-d₆, 80° C.) δ 8.44 (s, 1H), 7.95-7.82 (m, 1H), 7.73-7.56 (m, 2H), 7.29-7.17 (m, 1H), 7.10 (d, J=7.6 Hz, 2H), 6.25 (s, 1H), 5.13 (dd, J=10.6, 4.5 Hz, 1H), 4.34-4.15 (m, 2H), 3.95-3.82 (m, 1H), 3.75 (br. s., 1H), 3.27 (t, J=9.3 Hz, 1H), 3.21-3.13 (m, 1H), 2.24-2.12 (m, 2H), 2.01 (s, 6H), 1.83 (dd, J=14.9, 8.3 Hz, 1H), 1.58 (d, J=13.9 Hz, 1H), 0.77 (s, 3H), 0.68 (s, 3H), 0.56-0.44 (m, 4H). ESI-MS m/z calc. 576.2406, found 577.3 (M+1)⁺; Retention time: 4.02 minutes (LC method Y).

### Example 42: Preparation of Compound 52

### Step 1: 3-[[4-(2,6-Dimethylphenyl)-6-[(2R)-5-hydroxy-5-methyl-2-(spiro[2.3]hexan-5-ylamino)hexoxy]pyrimidin-2-yl]sulfamoyl]benzoic acid

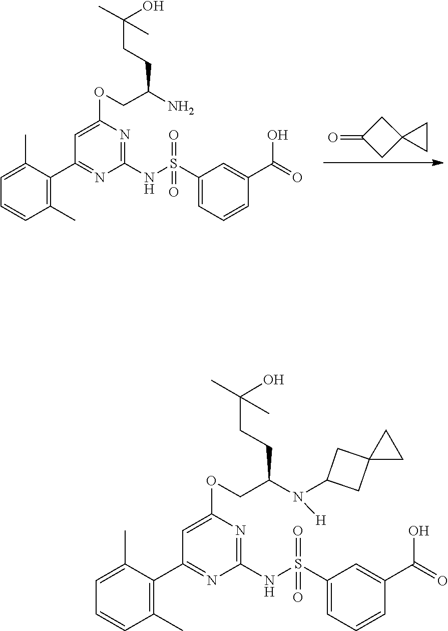 [SMILES: Cc1cccc(C)c1-c1cc(OC[C@H](N)CCC(C)(C)O)nc(NS(=O)(=O)c2cccc(C(=O)O)c2)n1 ; O=C1CC2(CC2)C1 ; Cc1cccc(C)c1-c1cc(OC[C@@H](CCC(C)(C)O)NC2CC3(CC3)C2)nc(NS(=O)(=O)c2cccc(C(=O)O)c2)n1]

3-[[4-[(2R)-2-Amino-5-hydroxy-5-methyl-hexoxy]-6-(2,6-dimethylphenyl)pyrimidin-2-yl]sulfamoyl]benzoic acid (50 mg, 0.0875 mmol), spiro[2.3]hexan-5-one (44 mg, 0.4577 mmol) and acetic acid (4.0128 mg, 3.8 μL, 0.0668 mmol) were stirred at room temperature for 1 hour in acetonitrile (1 mL) and Methanol (0.6 mL). Sodium cyanoborohydride (27.8 mg, 0.4424 mmol) was added and the reaction mixture was stirred for 2 hours. The reaction mixture was partitioned between ethyl acetate (50 mL) and saturated ammonium chloride (20 mL). The aqueous phase was separated and extracted with ethyl acetate (2×20 mL). The combined organic phases were washed with brine (10 mL), dried over sodium sulfate, filtered and concentrated under vacuo. The product was purified by normal phase chromatography (silica 12 g) using a gradient of 0-18% MeOH in DCM to provide 3-[[4-(2,6-dimethylphenyl)-6-[(2R)-5-hydroxy-5-methyl-2-(spiro[2.3]hexan-5-ylamino)hexoxy]pyrimidin-2-yl]sulfamoyl]benzoic acid (45.8 mg, 86%) as a white solid. ¹H NMR (400 MHz, DMSO-d₆) δ 8.40 (br. s., 1H), 7.98 (d, J=7.3 Hz, 1H), 7.89 (d, J=7.6 Hz, 1H), 7.66-7.55 (m, 1H), 7.54-7.44 (m, 1H), 7.18-7.07 (m, 1H), 7.02-6.95 (m, 2H), 6.20-6.05 (m, 1H), 4.15-4.08 (m, 2H), 2.21-2.07 (m, 2H), 2.05-1.98 (m, 2H), 1.89 (br. s., 6H), 1.54-1.40 (m, 2H), 1.32 (br. s., 3H), 1.23-1.16 (m, 2H), 0.97 (s, 6H), 0.77-0.70 (m, 2H), 0.39-0.30 (m, 2H), 0.30-0.19 (m, 2H). ESI-MS m/z calc. 608.2669, found 609.4 (M+1)⁺; Retention time: 1.41 minutes, LC method X.

### Step 2: (11R)-6-(2,6-Dimethylphenyl)-11-(3-hydroxy-3-methyl-butyl)-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (Compound 52)

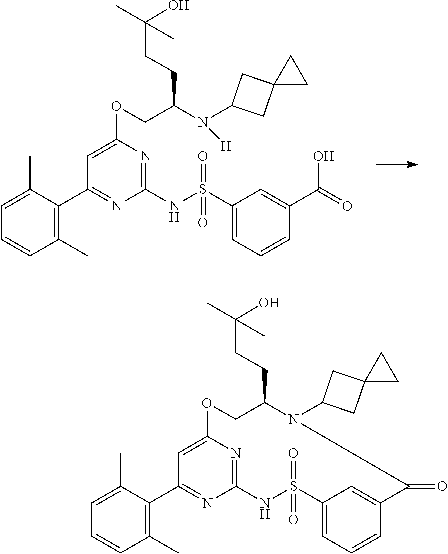

To a solution of 3-[[4-(2,6-dimethylphenyl)-6-[(2R)-5-hydroxy-5-methyl-2-(spiro[2.3]hexan-5-ylamino)hexoxy]pyrimidin-2-yl]sulfamoyl]benzoic acid (100 mg, 0.1643 mmol) in DMF (1.5000 mL) and EtOAc (5.5000 mL) was added TEA (108.90 mg, 0.15 mL, 1.0762 mmol). The solution was cooled down to 0° C. and propylphosphonic anhydride (50% solution in ethyl acetate) (213.80 mg, 0.4 mL, 0.3360 mmol) was slowly added. The reaction was stirred overnight at room temperature and then ethyl acetate was removed under vacuo. The product in DMF was directly injected on reverse phase column and purified by reverse phase chromatography (50 g C₁₈) using a gradient of 5% to 60% acetonitrile in water to provide after freeze drying (11R)-6-(2,6-dimethylphenyl)-11-(3-hydroxy-3-methyl-butyl)-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (50.1 mg, 51%) as a white fluffy solid. ¹H NMR (400 MHz, DMSO-d₆) δ 13.01 (br. s., 1H), 8.38 (br. s., 1H), 7.88 (br. s., 1H), 7.67 (br. s., 2H), 7.34-7.19 (m, 1H), 7.19-7.06 (m, 2H), 6.37 (br. s., 1H), 5.11 (dd, J=11.1, 3.8 Hz, 1H), 4.38 (t, J=11.1 Hz, 1H), 4.22 (quin, J=8.4 Hz, 1H), 4.04 (s, 1H), 3.69-3.58 (m, 1H), 3.31-3.17 (m, 2H), 2.25-1.86 (m, 8H), 1.75-1.49 (m, 2H), 1.33-1.17 (m, 1H), 0.90 (s, 6H), 0.84-0.73 (m, 1H), 0.56-0.40 (m, 4H). ESI-MS m/z calc. 590.2563, found 591.3 (M+1)⁺; Retention time: 3.97 minutes, LC method Y.

### Example 43: Preparation of Compound 53, Compound 54, Compound 55, Compound 56, Compound 57, and Compound 58

### Step 1: 3-[[4-[(2R)-2-[[3-(tert-Butoxycarbonylamino)cyclobutyl]amino]-3-[1-(trifluoromethyl)cyclopropyl]propoxy]-6-(2,6-dimethylphenyl)pyrimidin-2-yl]sulfamoyl]benzoic acid

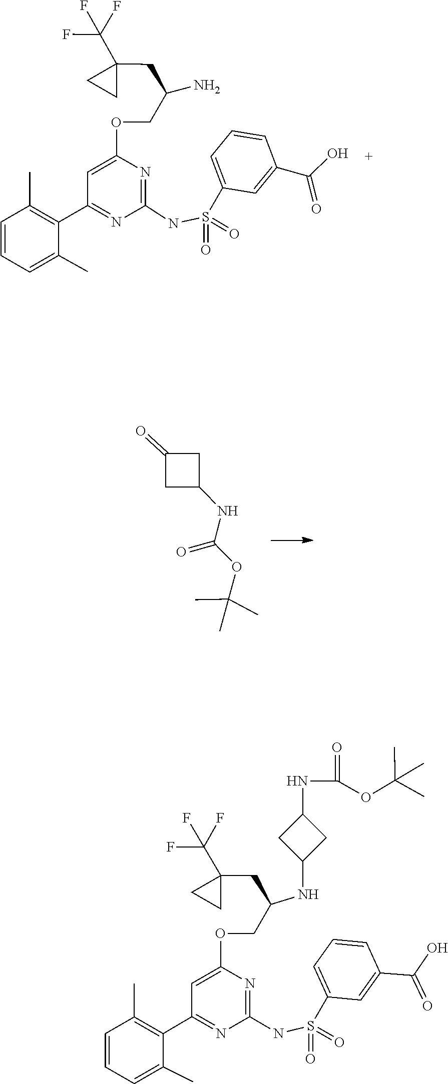

In a 200 mL flask, to a stirred solution of tert-butyl N-(3-oxocyclobutyl)carbamate (1.25 g, 6.749 mmol) in anhydrous dichloromethane (25 mL) was added 3-[[4-[(2R)-2-amino-3-[1-(trifluoromethyl)cyclopropyl]propoxy]-6-(2,6-dimethylphenyl)pyrimidin-2-yl]sulfamoyl]benzoic acid (hydrochloride salt) (3.25 g, 5.407 mmol) and stirred at ambient temperature for 30 min under nitrogen. Then sodium triacetoxyborohydride (3.65 g, 17.22 mmol) was added and the heterogeneous mixture was stirred at ambient temperature for 4 h. The reaction contents were partitioned between ice-cold hydrochloric acid (20 mL of 1.0 M, 20.00 mmol) and ethyl acetate (50 mL). The aqueous layer was extracted with ethyl acetate (2×50 mL). The combined organics were washed with brine (20 mL), dried over sodium sulfate, filtered and concentrated under reduced pressure to give crude 3-[[4-[(2R)-2-[[3-(tert-butoxycarbonylamino)cyclobutyl]amino]-3-[1-(trifluoromethyl)cyclopropyl]propoxy]-6-(2,6-dimethylphenyl)pyrimidin-2-yl]sulfamoyl]benzoic acid (hydrochloride salt) (4.20 g, 101%) as tan solid. It was used in the subsequent reaction without further purification. ESI-MS m/z calc. 733.2757, found 734.1 (M+1)⁺; Retention time: 1.34 minutes, LC method A.

### Step 2: tert-Butyl N-[3-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]cyclobutyl]carbamate

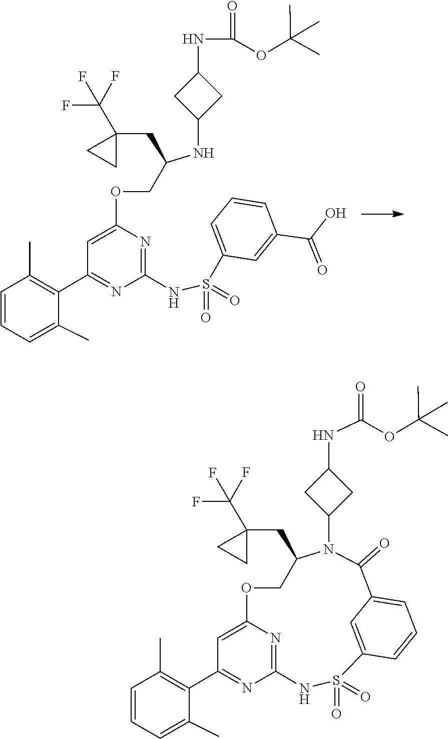 [SMILES: Cc1cccc(C)c1-c1cc(OC[C@@H](CC2(C(F)(F)F)CC2)NC2CC(NC(=O)OC(C)(C)C)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC(NC(=O)OC(C)(C)C)C1)[C@H](CC1(C(F)(F)F)CC1)CO2]

In a 500 mL flask, to a stirred solution of 3-[[4-[(2R)-2-[[3-(tert-butoxycarbonylamino)cyclobutyl]amino]-3-[1-(trifluoromethyl)cyclopropyl]propoxy]-6-(2,6-dimethylphenyl)pyrimidin-2-yl]sulfamoyl]benzoic acid (hydrochloride salt) (4.19 g, 5.440 mmol) in anhydrous DMF (190 mL), were added [dimethylamino(triazolo[4,5-b]pyridin-3-yloxy)methylene]-dimethyl-ammonium (Phosphorus Hexafluoride Ion) (3.11 g, 8.179 mmol) (HATU) and DIPEA (5.0 mL, 28.71 mmol), in that order, under nitrogen, at ambient temperature. After allowing the reaction to stir at ambient temperature for 16 h (overnight), the tea-colored reaction was concentrated under reduced pressure at 35° C. (water-bath temperature) and the residue was poured into an ice-cold aqueous solution of citric acid (65 mL of 10% w/v, 33.83 mmol). The product was extracted with ethyl acetate (3×100 mL). The combined organics were washed with brine (30 mL), dried over anhydrous sodium sulfate, filtered and concentrated under reduced pressure. The resulting brownish crude material was purified by flash chromatography (330 g silica gel, 0-10% methanol in methylene chloride over 30 min) to give tert-butyl N-[3-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]cyclobutyl]carbamate (3:1 mixture of isomers, 2.78 g, 61%), tan solid. ESI-MS m/z calc. 715.26514, found 716.1 (M+1)⁺; Retention time: 1.86 minutes, LC method A.

### Step 3: tert-Butyl N-[3-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-12-yl]cyclobutyl]carbamate, major diastereomer 1 (Compound 54), and tert-butyl N-[3-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-12-yl]cyclobutyl]carbamate, minor diastereomer 2 (Compound 55)

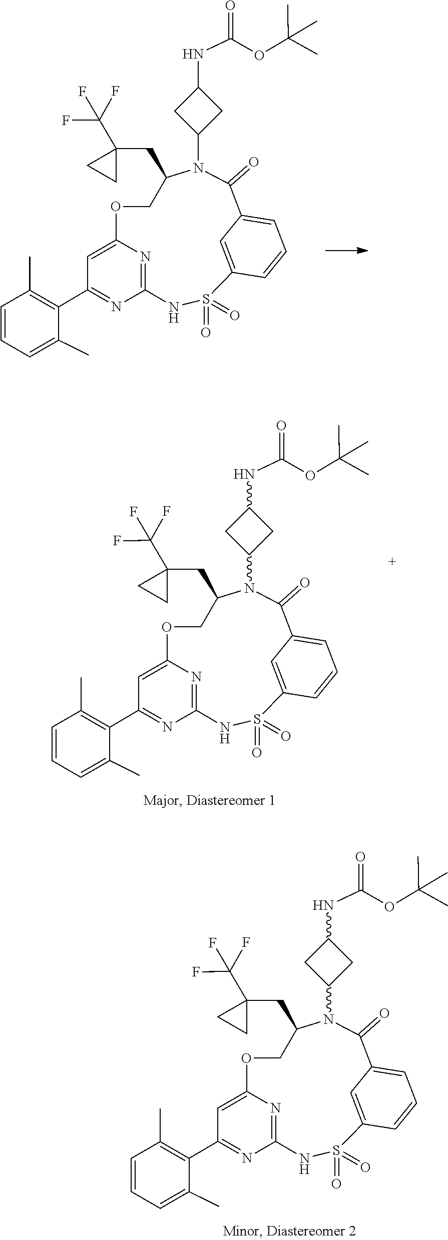 [SMILES: Cc1cccc(C)c1-c1cc2nc(n1)NS(=O)(=O)c1cccc(c1)C(=O)N(C1CC(NC(=O)OC(C)(C)C)C1)[C@H](CC1(C(F)(F)F)CC1)CO2 ; Cc1cccc(C)c1-c1cc2nc(n1)NS(=O)(=O)c1cccc(c1)C(=O)N(C1CC(NC(=O)OC(C)(C)C)C1)[C@H](CC1(C(F)(F)F)CC1)CO2 ; Cc1cccc(C)c1-c1cc2nc(n1)NS(=O)(=O)c1cccc(c1)C(=O)N(C1CC(NC(=O)OC(C)(C)C)C1)[C@H](CC1(C(F)(F)F)CC1)CO2]

tert-butyl N-[3-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]cyclobutyl]carbamate (mixture of isomers, 970 mg, 1.152 mmol) was taken up in DMSO (12 mL) and the solution was microfiltered through syringe filter disc and purified by preparative reverse phase HPLC (C₁₈) (5-99% acetonitrile in water over 30 min, HCl as a modifier) to furnish two isomers: Major diastereomer 1, tert-butyl N-[3-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-12-yl]cyclobutyl]carbamate (326 mg, 39%)¹H NMR (400 MHz, DMSO-d₆) δ 8.33 (d, J=1.8 Hz, 1H), 7.77 (d, J=6.8 Hz, 1H), 7.47 (s, 2H), 7.27 (d, J=7.5 Hz, 1H), 7.12 (t, J=7.6 Hz, 1H), 7.02 (d, J=7.5 Hz, 2H), 5.79 (s, 1H), 5.02 (dd, J=10.6, 4.4 Hz, 1H), 4.21-4.00 (m, 3H), 4.00-3.86 (m, 1H), 3.24 (q, J=9.3 Hz, 1H), 3.12 (q, J=9.4 Hz, 1H), 2.18-2.03 (m, 3H), 2.02-1.80 (m, 6H), 1.62-1.51 (m, 1H), 1.40 (s, 9H), 0.82-0.71 (m, 1H), 0.70-0.61 (m, 1H), 0.60-0.50 (m, 1H), 0.41-0.24 (m, 1H). ESI-MS m/z calc. 715.26514, found 716.1 (M+1)⁺; Retention time: 1.86 minutes (LC method A), and minor diastereomer 2, tert-butyl N-[3-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-12-yl]cyclobutyl]carbamate (150 mg, 18%). ¹H NMR (400 MHz, DMSO-d₆) δ 13.05 (s, 1H), 8.37 (s, 1H), 7.89 (s, 1H), 7.67 (s, 2H), 7.25 (t, J=7.9 Hz, 1H), 7.11 (d, J=8.0 Hz, 3H), 6.37 (s, 1H), 5.09 (dd, J=10.8, 4.4 Hz, 1H), 4.35 (t, J=11.3 Hz, 1H), 4.15-3.99 (m, 1H), 3.76-3.48 (m, 2H), 2.76-2.57 (m, 2H), 2.49-2.39 (m, 1H), 2.32-1.61 (m, 8H), 1.55 (dd, J=16.5, 9.5 Hz, 1H), 1.38 (s, 9H), 0.88-0.70 (m, 2H), 0.71-0.49 (m, 2H). ESI-MS m/z calc. 715.26514, found 716.1 (M+1)⁺; Retention time: 1.89 minutes (LC method A).

### Step 4: (11R)-12-(3-Aminocyclobutyl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-13-one, major diastereomer 1 (Compound 56)

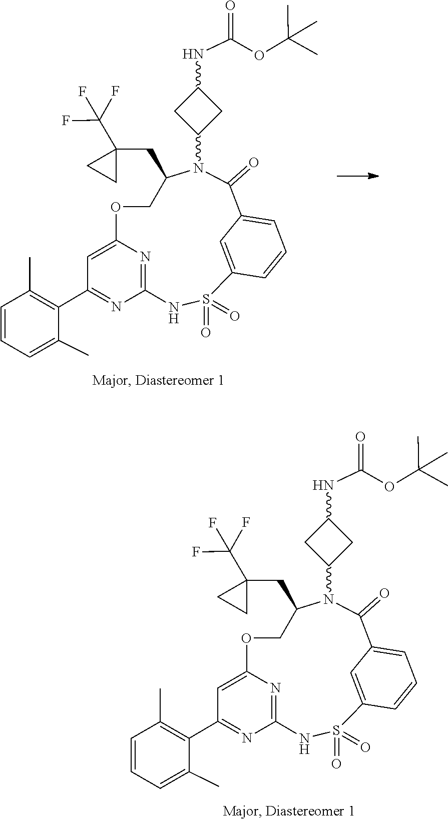 [SMILES: Cc1cccc(C)c1-c1cc2nc(n1)NS(=O)(=O)c1cccc(c1)C(=O)N(C1CC(NC(=O)OC(C)(C)C)C1)[C@H](CC1(C(F)(F)F)CC1)CO2 ; Cc1cccc(C)c1-c1cc2nc(n1)NS(=O)(=O)c1cccc(c1)C(=O)N(C1CC(NC(=O)OC(C)(C)C)C1)[C@H](CC1(C(F)(F)F)CC1)CO2]

To a stirred solution of tert-butyl N-[3-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-12-yl]cyclobutyl]carbamate (major diastereomer 1, 280 mg, 0.3912 mmol) in anhydrous dichloromethane (4 mL) was added hydrogen chloride in dioxane (1.2 mL of 4.0 M, 4.800 mmol) at ambient temperature under nitrogen. The pale-yellow solution was stirred at ambient temperature for 3 h, then concentrated under reduced pressure. The crude material was taken up in DMSO (3 mL) and the solution was micro-filtered through syringe filter disc and purified by preparative reverse phase HPLC (C₁₈) (10-99% acetonitrile in water over 30 min, HCl as a modifier) to furnish (11R)-12-(3-aminocyclobutyl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-13-one (hydrochloride salt) (255 mg, 99%) as a white solid. ¹H NMR (400 MHz, DMSO-d₆) δ 13.03 (s, 1H), 8.52 (s, 3H), 8.41 (s, 1H), 7.91 (d, J=7.4 Hz, 1H), 7.67 (dt, J=15.0, 7.7 Hz, 2H), 7.26 (t, J=7.6 Hz, 1H), 7.12 (d, J=7.7 Hz, 2H), 6.41 (s, 1H), 5.10 (dd, J=10.9, 4.4 Hz, 1H), 4.74 (t, J=8.8 Hz, 1H), 4.27 (t, J=11.3 Hz, 1H), 4.14-4.00 (m, 1H), 3.71-3.67 (m, 1H), 3.50-3.47 (m, 1H), 3.30 (dt, J=11.9, 8.4 Hz, 1H), 3.21 (dt, J=12.3, 8.4 Hz, 1H), 2.40-2.24 (m, 2H), 2.17-1.88 (m, 6H), 1.56 (dd, J=16.5, 9.2 Hz, 1H), 0.84 (dt, J=10.9, 5.4 Hz, 1H), 0.76 (dd, J=10.4, 5.4 Hz, 1H), 0.67-0.54 (m, 1H), 0.53-0.38 (m, 1H). ESI-MS m/z calc. 615.2127, found 616.2 (M+1)⁺; Retention time: 1.15 minutes (LC method A).

### Step 5: Methyl N-[3-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]cyclobutyl]carbamate, major diastereomer 1 (Compound 53)

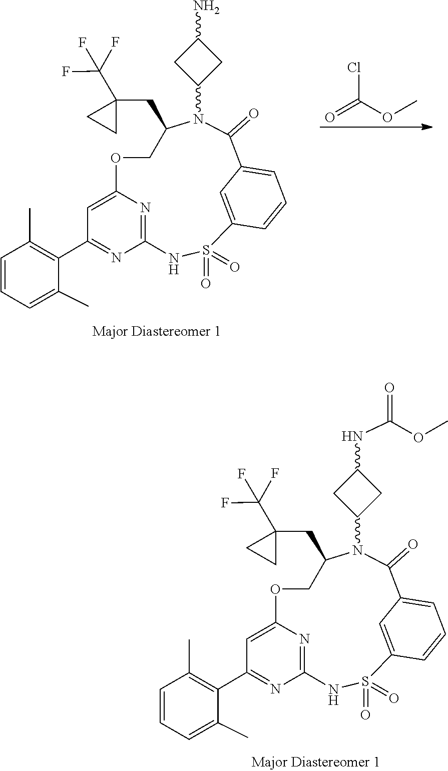

To a stirred solution of (11R)-12-(3-aminocyclobutyl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-13-one (hydrochloride salt) (major diastereomer 1, 50 mg, 0.07667 mmol) in anhydrous dichloromethane (0.8 mL) were added a solution of methyl chloroformate (10 mg, 0.1058 mmol) in dichloromethane (0.1 mL) and DIEA (70 μL, 0.4019 mmol), in that order, at ambient temperature. The vial was briefly purged with nitrogen and the capped vial was stirred at ambient temperature for 2 h. Then 3 drops of methanol were added, and the volatiles were removed under reduced pressure. The residue was taken up in DMSO (1 mL) and the solution was microfiltered through a syringe filter disc and purified by preparative reverse phase HPLC (C₁₈) using 1-99% acetonitrile in water over 15 min (HCl as a modifier) to furnish methyl N-[3-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]cyclobutyl]carbamate (9.7 mg, 19%) as a white solid. ¹H NMR (400 MHz, DMSO-d₆) δ 13.04 (s, 1H), 8.37 (s, 1H), 7.89 (s, 1H), 7.79-7.53 (m, 2H), 7.25 (t, J=7.8 Hz, 1H), 7.12 (d, J=7.6 Hz, 2H), 6.38 (s, 1H), 6.26 (d, J=6.9 Hz, 1H), 5.09 (dd, J=10.9, 4.4 Hz, 1H), 4.34-3.99 (m, 4H), 3.14 (q, J=20.1, 9.4 Hz, 2H), 2.27-1.82 (m, 9H), 1.47 (dd, J=16.5, 9.1 Hz, 1H), 0.89-0.71 (m, 2H), 0.71-0.59 (m, 1H), 0.57-0.44 (m, 1H). (OMe peak could be underneath broad water peak). ESI-MS m/z calc. 673.2182, found 674.1 (M+1)⁺; Retention time: 1.59 minutes, LC method A.

### Step 6: (11R)-12-(3-Aminocyclobutyl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-13-one, minor diastereomer 2 (Compound 57)

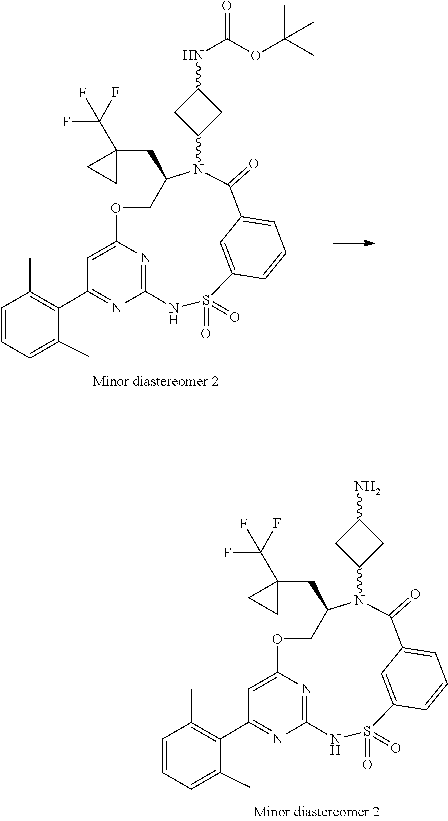 [SMILES: Cc1cccc(C)c1-c1cc2nc(n1)NS(=O)(=O)c1cccc(c1)C(=O)N(C1CC(NC(=O)OC(C)(C)C)C1)[C@H](CC1(C(F)(F)F)CC1)CO2 ; Cc1cccc(C)c1-c1cc2nc(n1)NS(=O)(=O)c1cccc(c1)C(=O)N(C1CC(N)C1)[C@H](CC1(C(F)(F)F)CC1)CO2]

To a stirred solution of tert-butyl N-[3-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-12-yl]cyclobutyl]carbamate (130 mg, 0.1816 mmol) in anhydrous dichloromethane (2 mL) was added hydrogen chloride in dioxane (600 μL of 4.0 M, 2.400 mmol) at ambient temperature under nitrogen. The solution was stirred at ambient temperature for 3 h, then concentrated under reduced pressure. The crude material was taken up in DMSO (2.5 mL) and the solution was micro-filtered through a syringe filter disc and purified by preparative reverse phase HPLC (C₁₈) (10-99% acetonitrile in water over 30 min, HCl as a modifier, big column 50×100 mm, one injection) to furnish (11R)-12-(3-aminocyclobutyl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-13-one (hydrochloride salt) (118 mg, 97%) as a white solid. ¹H NMR (400 MHz, DMSO-d₆) δ 13.04 (s, 1H), 8.85-8.08 (m, 4H), 7.91 (d, J=7.5 Hz, 1H), 7.84-7.55 (m, 2H), 7.26 (t, J=7.6 Hz, 1H), 7.12 (d, J=7.6 Hz, 2H), 6.37 (s, 1H), 5.13 (dd, J=10.9, 4.4 Hz, 1H), 4.30 (t, J=11.3 Hz, 1H), 4.15-3.99 (m, 1H), 3.78 (t, J=8.4 Hz, 1H), 3.07-2.86 (m, 2H), 2.58 (d, J=9.0 Hz, 2H), 2.32-1.67 (m, 7H), 1.56 (dd, J=16.6, 9.3 Hz, 1H), 0.93-0.69 (m, 2H), 0.69-0.45 (m, 2H). (one of the aliphatic protons likely underneath broad water peak) ESI-MS m/z calc. 615.2127, found 616.1 (M+1)⁺; Retention time: 1.18 minutes (LC method A).

### Step 7: Methyl N-[3-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]cyclobutyl]carbamate, minor diastereomer 2 (Compound 58)

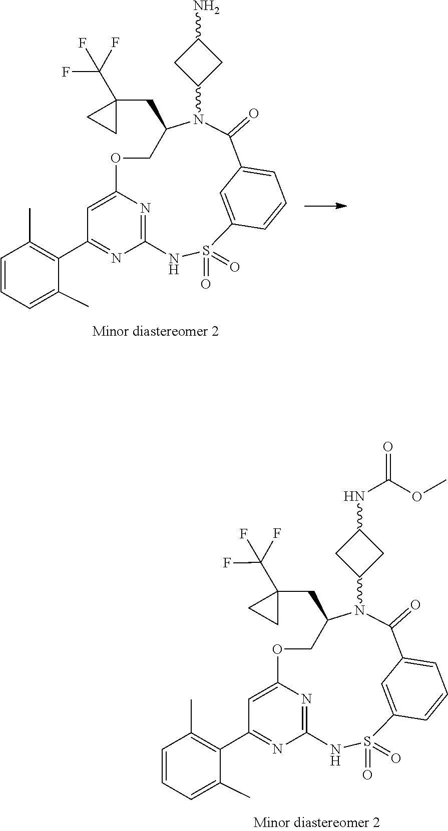 [SMILES: Cc1cccc(C)c1-c1cc2nc(n1)NS(=O)(=O)c1cccc(c1)C(=O)N(C1CC(N)C1)[C@H](CC1(C(F)(F)F)CC1)CO2 ; COC(=O)NC1CC(N2C(=O)c3cccc(c3)S(=O)(=O)Nc3nc(cc(-c4c(C)cccc4C)n3)OC[C@H]2CC2(C(F)(F)F)CC2)C1]

To a stirred solution of (11R)-12-(3-aminocyclobutyl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-13-one (hydrochloride salt) (6 mg, 0.009201 mmol) in anhydrous dichloromethane (0.5 mL) were added a solution of methyl chloroformate (1 mg, 0.01058 mmol) in dichloromethane (0.1 mL) and pyridine (5 μL, 0.06182 mmol), in that order, at ambient temperature. The vial was briefly purged with nitrogen and the capped vial was stirred at ambient temperature for 1 h. Then 3 drops of methanol was added and the volatiles were removed under reduced pressure. The residue was taken up in DMSO (1 mL) and the solution was microfiltered through a Whatman 0.45 uM PTFE syringe filter disc and purified by preparative reverse phase HPLC (C₁₈) using 1-99% acetonitrile in water over 15 min (HCl as a modifier). The desired fractions were dried in Genevac to furnish methyl N-[3-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]cyclobutyl]carbamate (2.7 mg, 43%) as white solid. ESI-MS m/z calc. 673.2182, found 674.1 (M+1)⁺; Retention time: 1.63 minutes (LC method A).

### Example 44: Preparation of Compound 59

### Step 6: isopropyl N-[3-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]cyclobutyl]carbamate (Compound 59)

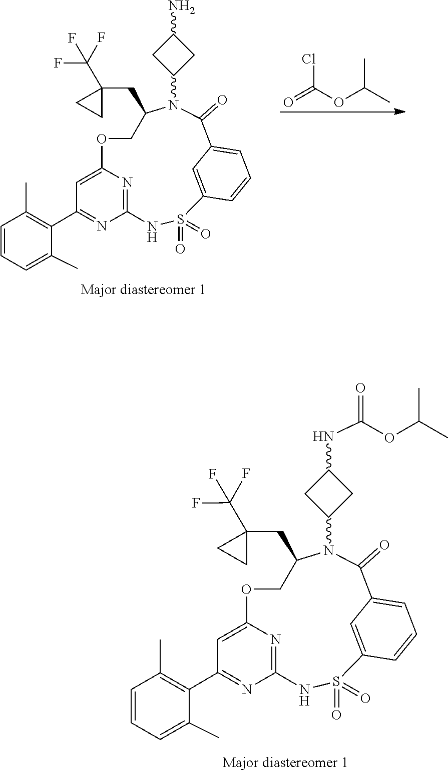

To a stirred solution of (11R)-12-(3-aminocyclobutyl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-13-one (hydrochloride salt) (major diastereomer 1, 60 mg, 0.09201 mmol) in anhydrous dichloromethane (0.8 mL) were added a solution of isopropyl chloroformate (50 mg of 30% w/w, 0.1224 mmol) in dichloromethane (0.1 mL) and DIEA (100 μL, 0.5741 mmol), in that order, at ambient temperature. The vial was briefly purged with nitrogen and the capped vial was stirred at ambient temperature for 2 h. Then 3 drops of methanol were added, and the volatiles were removed under reduced pressure. The residue was taken up in DMSO (1 mL) and the solution was microfiltered through a syringe filter disc and purified by preparative reverse phase HPLC (C₁₈) using a gradient of acetonitrile in water (1 to 99% over 15 min HCl as a modifier) to give isopropyl N-[3-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]cyclobutyl]carbamate (18 mg, 28%) as white solid. ¹H NMR (400 MHz, DMSO-d₆) δ 13.02 (s, 1H), 8.36 (s, 1H), 7.89 (s, 1H), 7.78-7.56 (m, 2H), 7.42 (d, J=7.1 Hz, 1H), 7.25 (d, J=8.7 Hz, 1H), 7.12 (s, 2H), 6.38 (s, 1H), 5.07 (d, J=10.4 Hz, 1H), 4.76 (hept, J=6.2 Hz, 1H), 4.25-4.05 (m, 4H), 3.78-3.65 (m, 2H), 3.66-3.58 (m, 1H), 3.23-3.05 (m, 2H), 2.21-2.05 (m, 5H), 2.02-1.87 (m, 3H), 1.46 (dd, J=16.5, 9.1 Hz, 1H), 1.18 (d, J=6.2 Hz, 4H), 0.87-0.70 (m, 2H), 0.69-0.57 (m, 1H), 0.56-0.43 (m, 1H). ESI-MS m/z calc. 701.2495, found 702.1 (M+1)⁺; Retention time: 1.76 minutes (LC method A).

### Example 45: Preparation of Compound 60

### Step 3: Isopropyl N-[3-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]cyclobutyl]carbamate, minor diastereomer 2 (Compound 60)

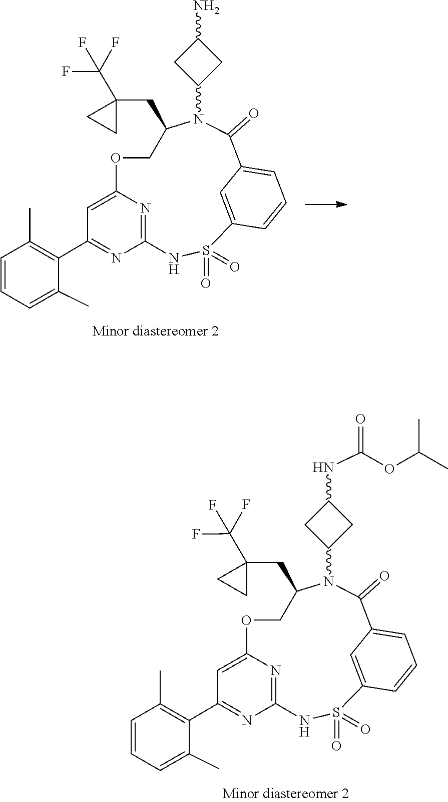 [SMILES: Cc1cccc(C)c1-c1cc2nc(n1)NS(=O)(=O)c1cccc(c1)C(=O)N(C1CC(N)C1)[C@H](CC1(C(F)(F)F)CC1)CO2 ; Cc1cccc(C)c1-c1cc2nc(n1)NS(=O)(=O)c1cccc(c1)C(=O)N(C1CC(NC(=O)OC(C)C)C1)[C@H](CC1(C(F)(F)F)CC1)CO2]

To a stirred solution of (11R)-12-(3-aminocyclobutyl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-13-one (hydrochloride salt) (minor diastereomer 2, 12 mg, 0.01840 mmol) in anhydrous dichloromethane (0.5 mL) were added a solution of isopropyl chloroformate (11 mg of 30% w/w, 0.02693 mmol) in dichloromethane (0.1 mL) and DIEA (20 μL, 0.1148 mmol), in that order, at ambient temperature. The vial was briefly purged with nitrogen and the capped vial was stirred at ambient temperature for 40 min. Then 3 drops of methanol were added and the volatiles were removed under reduced pressure. The residue was taken up in DMSO (1 mL) and the solution was microfiltered through a syringe filter disc and purified by preparative reverse phase HPLC (C₁₈) using a gradient of acetonitrile in water (1 to 99% over 15 min HCl as a modifier) to give N-[3-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]cyclobutyl]carbamate (4.4 mg, 34%) as a white solid. ESI-MS m/z calc. 701.2495, found 702.1 (M+1)⁺; Retention time: 1.8 minutes (LC method A).

### Example 46: Preparation of Compound 61

### Step 1: (11R)-12-[3-(dimethylamino)cyclobutyl]-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-13-one, major diastereomer 1 (Compound 61)

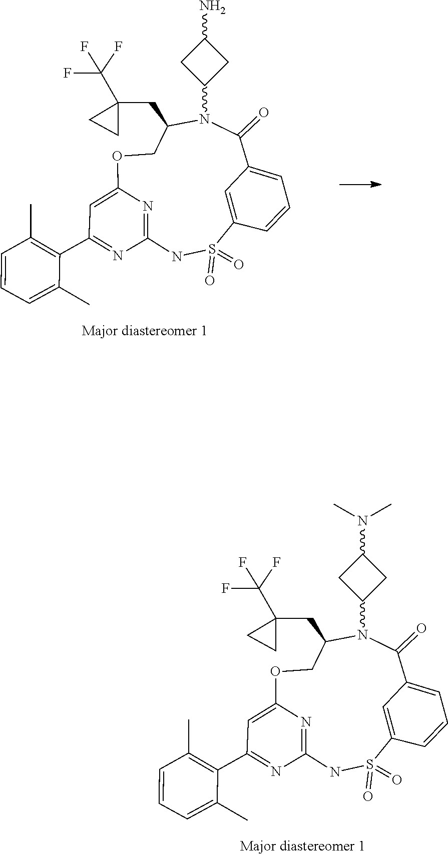

In a 4 mL vial, to a solid (11R)-12-(3-aminocyclobutyl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-13-one (hydrochloride salt) (major diastereomer 1, 10 mg, 0.01518 mmol) were added formaldehyde (0.25 mL, 9.075 mmol) and formic acid (0.20 mL, 5.301 mmol), in that order at ambient temperature. The screw-capped vial was capped under nitrogen and stirred at 95° C. for 16 h (overnight). The reaction mixture was allowed to cool to room temperature, and diluted with methanol (0.2 mL) and DMSO (0.5 mL), microfiltered, and purified by preparative reverse-phase HPLC (Cis column, 1-70% acetonitrile in water, HCl modifier, 15 min run) to afford (11R)-12-[3-(dimethylamino)cyclobutyl]-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-13-one (hydrochloride salt) (6 mg, 58%) as a white solid. ¹H NMR (400 MHz, DMSO-d₆) δ 13.0 (s, 1H), 8.41 (s, 1H), 7.95-7.84 (m, 1H), 7.76-7.59 (m, 2H), 7.24 (d, J=7.7 Hz, 1H), 7.12 (d, J=7.6 Hz, 2H), 6.36 (s, 1H), 5.08 (d, J=9.2 Hz, 1H), 4.32-4.13 (m, 2H), 4.12-4.01 (m, 1H), 3.30 (s, 3H), 3.03 (s, 3H), 2.21-2.06 (m, 4H), 2.05-1.77 (m, 6H), 1.48 (dd, J=16.3, 8.7 Hz, 2H), 0.87-0.80 (m, 2H), 0.78-0.72 (m, 1H), 0.67-0.57 (m, 1H), 0.57-0.45 (m, 1H). ESI-MS m/z calc. 643.244, found 644.2 (M+1)⁺; Retention time: 1.18 minutes (LC method A).

### Example 47: Preparation of Compound 62

### Step 1: Methyl 6-[[4-chloro-6-(2,6-dimethylphenyl)pyrimidin-2-2yl]sulfamoyl]pyrazine-2-carboxylate

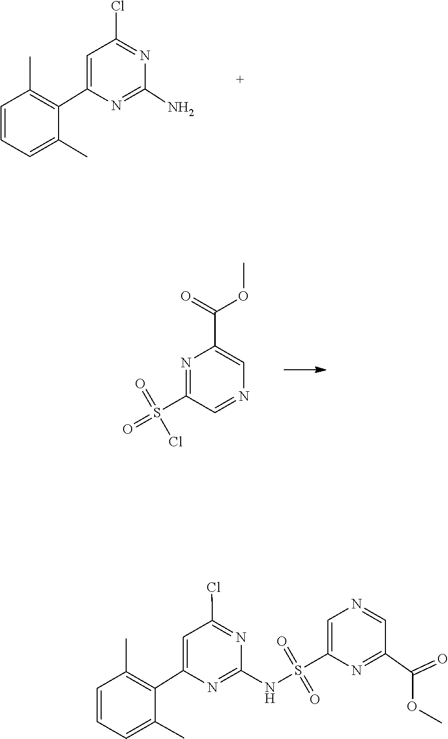 [SMILES: Cc1cccc(C)c1-c1cc(Cl)nc(N)n1 ; COC(=O)c1cncc(S(=O)(=O)Cl)n1 ; COC(=O)c1cncc(S(=O)(=O)Nc2nc(Cl)cc(-c3c(C)cccc3C)n2)n1]

4-Chloro-6-(2,6-dimethylphenyl)pyrimidin-2-amine (4.15 g, 17.758 mmol) was dissolved in MeTHF (25 mL) and was cooled in an iced bath. Methyl 6-chlorosulfonylpyrazine-2-carboxylate (13.64 g, 57.642 mmol) in MeTHF (25 mL) was added at 0° C. To the cold solution, lithium tert-butoxide (17 mL of 3.1 M, 52.700 mmol) (in heptane) was added dropwise. The ice bath was removed, and the mixture was stirred for 3 hours at room temperature. 1N aqueous hydrochloric acid solution (50 mL) was added and the phases was separated. The aqueous phase was extracted with MeTHF (50 mL) and the organic phase were combined, washed with brine (50 mL), dried over sodium sulfate, filtered and concentrated. The residue was purified by silica-gel column chromatography on a 330 g column, eluting from 0% to 30% of ethyl acetate in heptanes to afford methyl 6-[[4-chloro-6-(2,6-dimethylphenyl)pyrimidin-2-yl]sulfamoyl]pyrazine-2-carboxylate (4.85 g, 18%) as an off-white solid. ¹H NMR (300 MHz, CDCl₃) δ 9.58 (s, 1H), 9.44 (s, 1H), 7.23-7.17 (m, 1H), 7.06 (d, J=7.9 Hz, 2H), 6.91 (s, 1H), 4.03 (s, 3H), 1.95 (s, 6H). ESI-MS m/z calc. 433.06116, found 434.1 (M+1)⁺; Retention time: 1.98 minutes; LC method K.

### Step 2: 6-[[4-Chloro-6-(2,6-dimethylphenyl)pyrimidin-2-yl]sulfamoyl]pyrazine-2-carboxylic acid

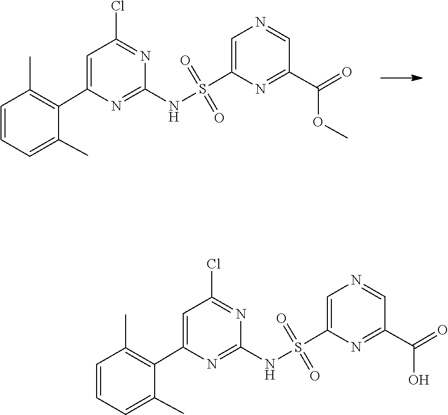 [SMILES: COC(=O)c1cncc(S(=O)(=O)Nc2nc(Cl)cc(-c3c(C)cccc3C)n2)n1 ; Cc1cccc(C)c1-c1cc(Cl)nc(NS(=O)(=O)c2cncc(C(=O)O)n2)n1]

A mixture of methyl 6-[[4-chloro-6-(2,6-dimethylphenyl)pyrimidin-2-yl]sulfamoyl]pyrazine-2-carboxylate (4.85 g, 10.136 mmol) in THE (125 mL) and Water (125 mL) was treated with lithium hydroxide mono hydrate (1.3 g, 30.979 mmol) and stirred vigorously at room temperature for 3 hours. 1N Aqueous sodium hydroxide solution (125 mL) was added and extracted with diethyl ether (125 mL) and 2-MeTHF (125 mL). The aqueous phase was acidified to pH<3 with 3N aqueous hydrochloric acid solution and extracted with ethyl acetate (3×125 mL). The combined organic layers were washed with brine (125 mL), dried over sodium sulfate, filtered and concentrated under reduced pressure to afford 6-[[4-chloro-6-(2,6-dimethylphenyl)pyrimidin-2-yl]sulfamoyl]pyrazine-2-carboxylic acid (4.4 g, 87%) as a yellow solid. ¹H NMR (300 MHz, DMSO-d₆) δ 13.55-12.73 (m, 2H), 9.34 (s, 1H), 9.32 (s, 1H), 7.30 (s, 1H), 7.26-7.16 (m, 1H), 7.07 (d, J=7.6 Hz, 2H), 1.82 (s, 6H). ESI-MS m/z calc. 419.0455, found 420.1 (M+1)⁺; Retention time: 2.59 minutes; LC method U.

### Step 3: 6-[[4-(2,6-Dimethylphenyl)-6-[(2R)-4-methyl-2-(spiro[2.3]hexan-5-ylamino)pentoxy]pyrimidin-2-yl]sulfamoyl]pyrazine-2-carboxylic acid

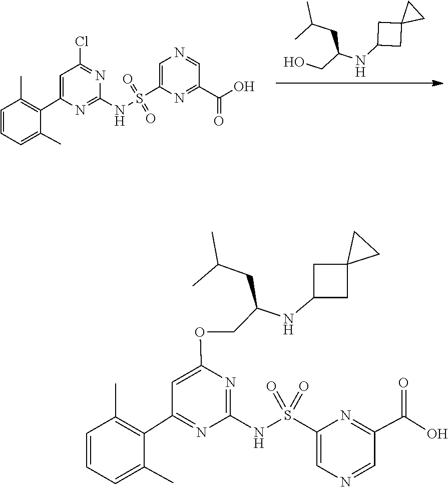 [SMILES: Cc1cccc(C)c1-c1cc(Cl)nc(NS(=O)(=O)c2cncc(C(=O)O)n2)n1 ; CC(C)C[C@H](CO)NC1CC2(CC2)C1 ; Cc1cccc(C)c1-c1cc(OC[C@@H](CC(C)C)NC2CC3(CC3)C2)nc(NS(=O)(=O)c2cncc(C(=O)O)n2)n1]

In a 100 mL flask, 6-[[4-chloro-6-(2,6-dimethylphenyl)pyrimidin-2-yl]sulfamoyl]pyrazine-2-carboxylic acid (272 mg, 0.6479 mmol) and (2R)-4-methyl-2-(spiro[2.3]hexan-5-ylamino)pentan-1-ol (hydrochloride salt) (153 mg, 0.6545 mmol) were charged under nitrogen with anhydrous THE (2 mL) (suspension). Sodium tert-butoxide (272 mg, 2.830 mmol) was added (slight exotherm). The reaction turned into a thick gel. More THE (2 mL) was added and the suspension was stirred at room temperature for 5.5 hours. The mixture was partitioned between ethylacetate (30 mL) and aqueous 1M HCl (30 mL) and brine (20 mL). After separation, the aqueous phase was further extracted with EtOAc (2×30 mL). The combined extracts were dried over sodium sulfate and the solvents evaporated to give a crude material. The material was dissolved in DMSO (3 mL). The solution was microfiltered through a syringe filter disc and purified by reverse phase preparative HPLC (C₁₈) using a gradient of acetonitrile in water (1 to 99% over 15 min) and HCl as a modifier. Evaporation gave 6-[[4-(2,6-dimethylphenyl)-6-[(2R)-4-methyl-2-(spiro[2.3]hexan-5-ylamino)pentoxy]pyrimidin-2-yl]sulfamoyl]pyrazine-2-carboxylic acid (hydrochloride salt) (141 mg, 35%) as an off-white solid. ESI-MS m/z calc. 580.24677, found 581.77 (M+1)⁺; Retention time: 1.26 minutes (LC method A).

### Step 4: (11R)-6-(2,6-Dimethylphenyl)-11-isobutyl-2,2-dioxo-12-spiro[2.3]hexan-5-yl-9-oxa-2λ⁶-thia-3,5,12,16,18,19-hexazatricyclo[12.3.1.14,8]nonadeca-1(18),4(19),5,7,14,16-hexaen-13-one (Compound 62)

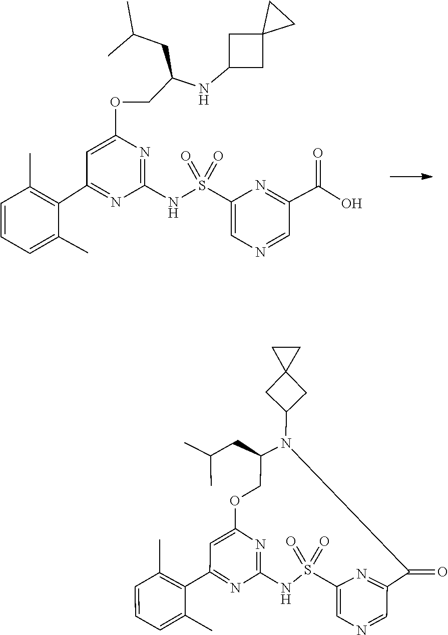

A 20 mL flask was charged under nitrogen with HATU (199 mg, 0.5234 mmol), anhydrous DMF (9 mL) and DIEA (0.22 mL, 1.263 mmol). A solution of 6-[[4-(2,6-dimethylphenyl)-6-[(2R)-4-methyl-2-(spiro[2.3]hexan-5-ylamino)pentoxy]pyrimidin-2-yl]sulfamoyl]pyrazine-2-carboxylic acid (hydrochloride salt) (141 mg, 0.2285 mmol) in anhydrous DMF (6 mL) was added dropwise through syringe over a period of 4 minutes. The mixture was stirred at room temperature for 12 hours. The mixture was concentrated and diluted with DMSO (2 mL). The solution was microfiltered through a syringe filter disc and purified by reverse phase preparative HPLC (C₁₈) using a gradient of acetonitrile in water (1 to 99% over 15 min) and HCl as a modifier. Evaporation gave a residue that was triturated in DCM/hexanes. Evaporation of the solvents gave (11R)-6-(2,6-dimethylphenyl)-11-isobutyl-2,2-dioxo-12-spiro[2.3]hexan-5-yl-9-oxa-2λ⁶-thia-3,5,12,16,18,19-hexazatricyclo[12.3.1.14,8]nonadeca-1(18),4(19),5,7,14,16-hexaen-13-one (85 mg, 65%) as an off-white solid. ¹H NMR (500 MHz, DMSO-d₆) δ 13.26 (broad s, 1H), 9.23 (s, 1H), 9.09 (s, 1H), 7.29 (t, J=7.6 Hz, 1H), 7.15 (d, J=7.7 Hz, 2H), 6.41 (s, 1H), 5.55 (dd, J=9.7, 4.7 Hz, 1H), 4.33 (p, J=8.6 Hz, 1H), 4.26 (t, J=10.6 Hz, 1H), 3.51 (tt, J=11.1, 4.1 Hz, 1H), 3.33-3.30 (m, 1H overlapped with water), 2.31-1.88 (m, 8H), 1.72 (ddd, J=14.2, 10.7, 3.2 Hz, 1H), 1.45-1.33 (m, 1H), 1.30-1.15 (m, 2H), 0.75 (d, J=6.6 Hz, 3H), 0.57-0.50 (m, 2H), 0.50-0.42 (m, 2H), 0.33 (d, J=6.4 Hz, 3H). ESI-MS m/z calc. 562.2362, found 563.33 (M+1)⁺; Retention time: 1.96 minutes (LC method A).

### Example 48: Preparation of Compound 63

### Step 1: (11R)-12-(3-aminocyclobutyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one, stereoisomer 1 and 2

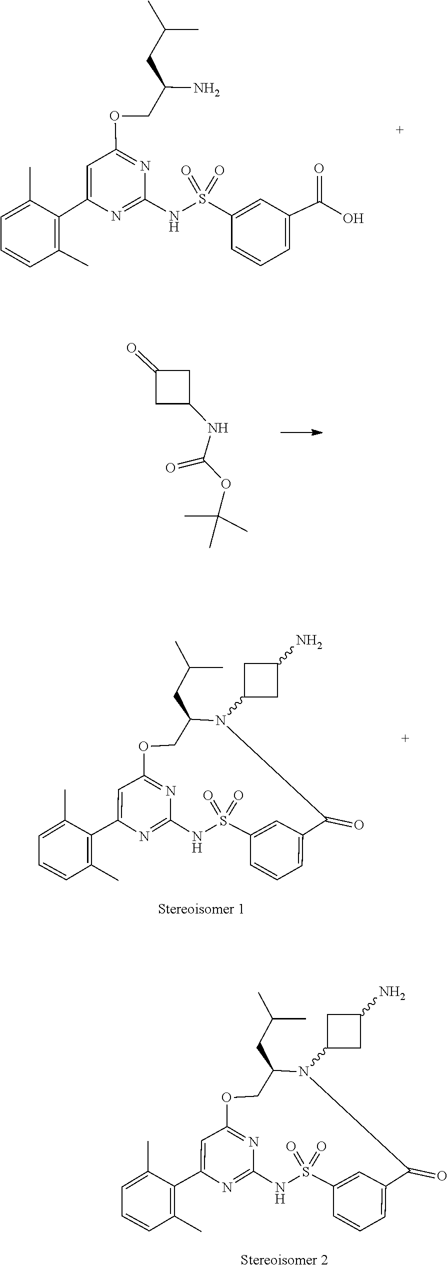

3-[[4-[(2R)-2-Amino-4-methyl-pentoxy]-6-(2,6-dimethylphenyl)pyrimidin-2-yl]sulfamoyl]benzoic acid (hydrochloride salt) (300 mg, 0.5607 mmol) was combined with tert-butyl N-(3-oxocyclobutyl)carbamate (155 mg, 0.8368 mmol) in dichloromethane (1 mL) and stirred for 10 minutes at room temperature at which point the starting materials had almost completely dissolved. Sodium triacetoxyborohydride (350 mg, 1.651 mmol) was added and the reaction was stirred at room temperature for 1 hour. An additional portion of sodium triacetoxyborohydride (350 mg, 1.651 mmol) was added and the reaction was stirred at room temperature for an additional hour. The reaction mixture was then partitioned between 0.5 M HCl and ethyl acetate. The aqueous layer was extracted an additional three times ethyl acetate. The combined organics were washed with brine, dried over sodium sulfate and concentrated to give crude 3-[[4-[(2R)-2-[[3-(tert-butoxycarbonylamino)cyclobutyl]amino]-4-methyl-pentoxy]-6-(2,6-dimethylphenyl)pyrimidin-2-yl]sulfamoyl]benzoic acid, which was used in the next stage without further purification. ESI-MS m/z calc. 667.30396, found 668.5 (M+1)⁺; Retention time: 0.53 minutes; LC method D.

The product was combined in DMF (25 mL) with HATU (320 mg, 0.8416 mmol), and DIPEA (490 μL, 2.813 mmol) was added. The reaction mixture was stirred at room temperature for 6 hours. The reaction mixture was then partitioned between 1M HCl and ethyl acetate. The layers were separated, and the aqueous layer was extracted an additional three times with ethyl acetate. The combined organics were washed with brine, dried over sodium sulfate, and concentrated. The resulting crude material was purified by reverse phase chromatography (1-99% ACN in water, HCl modifier, initially shallow gradient—split between two runs) to give the two isomeric conformations of the cyclobutane ring separately Stereoisomer 1 (first eluting), tert-butyl N-[3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]carbamate (24 mg, 7%), ESI-MS m/z calc. 649.2934, found 650.3 (M+1)⁺; Retention time: 0.76 minutes; LC method D; and stereoisomer 2, tert-butyl N-[3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]carbamate (21 mg, 6%), ESI-MS m/z calc. 649.2934, found 650.3 (M+1)⁺; Retention time: 0.78 minutes; LC method D

The separately isolated products from above were separately dissolved in dichloromethane (0.25 mL) and HCl (200 μL of 4 M, 0.8000 mmol) was added. After stirring at room temperature, for 45 minutes, the reaction mixtures were concentrated to give as a white solid (11R)-12-(3-aminocyclobutyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (22 mg, 7%), Stereoisomer 1, ESI-MS m/z calc. 549.24097, found 550.5 (M+1)⁺; Retention time: 0.5 minutes; LC method D; and (11R)-12-(3-aminocyclobutyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (11 mg, 3%) (Stereoisomer 2), ESI-MS m/z calc. 549.24097, found 550.5 (M+1)⁺; Retention time: 0.48 minutes; LC method D.

### Step 2: Propan-2-yl N-{3-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]cyclobutyl}carbamate (Compound 63)

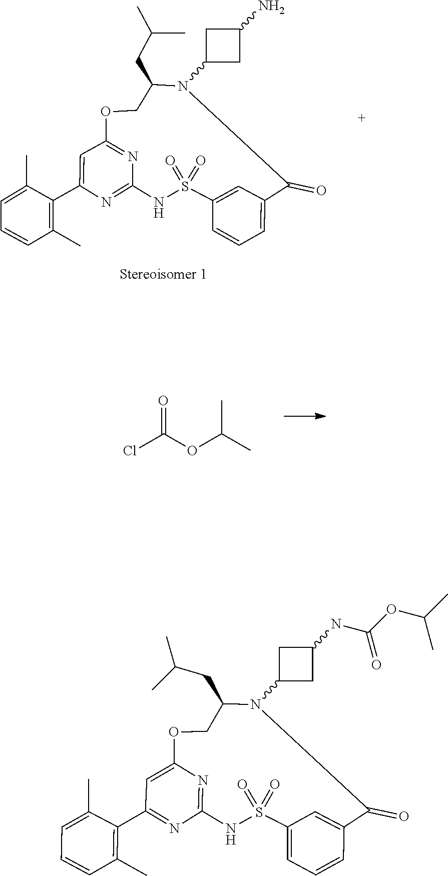

(11R)-12-(3-aminocyclobutyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (11 mg, 0.01877 mmol) (Stereoisomer 1) was dissolved in DCM (0.5 mL), and DIPEA (approximately 12.13 mg, 16.35 μL, 0.09385 mmol) and isopropyl chloroformate (approximately 18.77 μL of 2 M, 0.03754 mmol) were added sequentially. The reaction mixture was stirred for 15 minutes at room temperature, then was quenched with two drops of 1M HCl and partially concentrated. The resulting crude was dissolved in 1:1 methanol/DMSO, filtered, and purified by reverse phase HPLC (1-99% ACN in water, HCl modifier, 15 min run) to give the indicated propan-2-yl N-{3-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]cyclobutyl}carbamate (7.5 mg, 63%). ESI-MS m/z calc. 635.2778, found 636.4 (M+1)⁺; Retention time: 1.79 minutes; LC method A.

### Example 49: Preparation of Compound 64

### Step 1: Propan-2-yl N-{3-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]cyclobutyl}carbamate (stereoisomer Compounds 64 and 65)

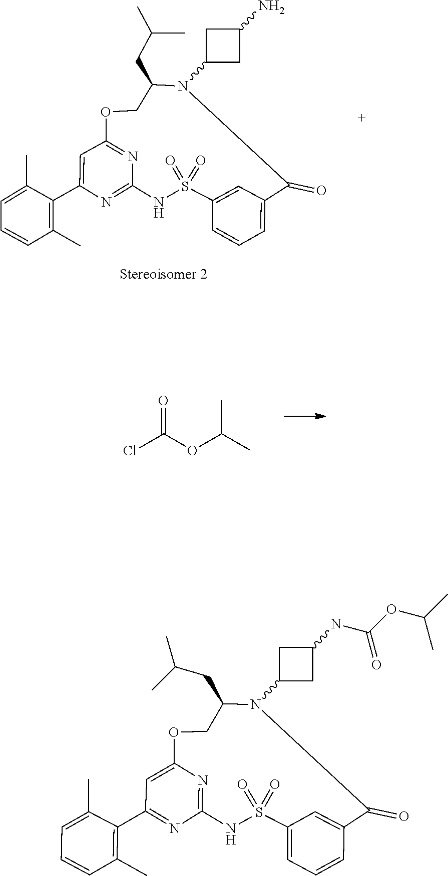

(11R)-12-(3-aminocyclobutyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (5 mg, 0.008530 mmol) (Stereoisomer 2 was dissolved in DCM (0.5 mL), and DIPEA (approximately 5.512 mg, 7.429 μL, 0.04265 mmol) and isopropyl chloroformate (approximately 8.530 μL of 2 M, 0.01706 mmol) were added sequentially. The reaction mixture was stirred for 15 minutes at room temperature, then was quenched with two drops of 1M HCl and partially concentrated. The resulting crude was dissolved in 1:1 methanol/DMSO, filtered, and purified by reverse phase HPLC (1-99% ACN in water, HCl modifier, 15 min run) to give the indicated propan-2-yl N-{3-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]cyclobutyl}carbamate (1.6 mg, 29%). ESI-MS m/z calc. 635.2778, found 636.4 (M+1)⁺; Retention time: 1.83 minutes; LC method A.

### Example 50: Preparation of Compounds 65 and 66

### Step 1: (11R)-6-(2,6-Dimethylphenyl)-11-isobutyl-2,2-dioxo-12-(3-oxocyclobutyl)-9-oxa-2λ⁶-thia-3,5,12,19-tetrazatricyclo[12.3.1.14,8]nonadeca-1(18),4(19),5,7,14,16-hexaen-13-one

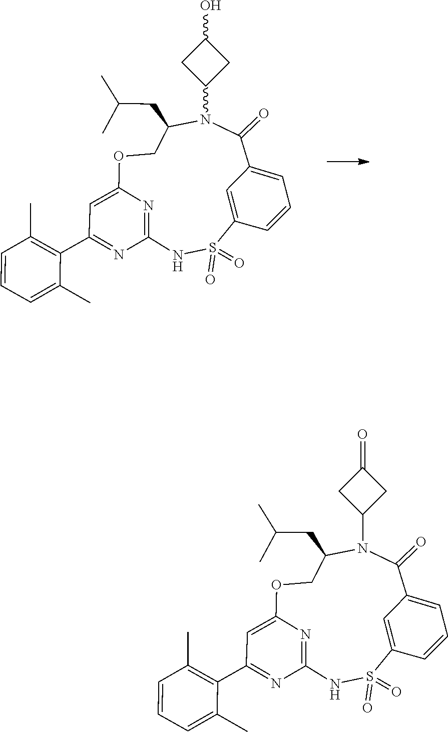

To a stirred solution of (11R)-6-(2,6-dimethylphenyl)-12-(3-hydroxycyclobutyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (1.56 g, 2.833 mmol) in anhydrous dichloromethane (20 mL) was added (1,1-diacetoxy-3-oxo-1λ⁵,2-benziodoxol-1-yl) acetate (1.445 g, 3.407 mmol) (Dess-Martin Periodinane) at 0-5° C. (ice-water bath) under nitrogen. After 30 min, the reaction was allowed to warm to ambient temperature and stirring continued for 14 h (overnight). The reaction was diluted with ether (100 mL) and saturated aqueous sodium bicarbonate (30 mL) was added very slowly (to mitigate CO₂ gas evolution). Then 10% sodium thiosulfate (25 mL) was added and stirred at ambient temperature for 20 min. The layers were separated, and the aqueous layer was extracted with ether (2×30 mL). The combined organics were washed with brine (30 mL), dried over anhydrous sodium sulfate, filtered and concentrated under reduced pressure to obtain a crude material which was purified by silica gel chromatography (120 g silica gel column, 5-60% EtOAc in hexanes over 30 min,) to obtain (11R)-6-(2,6-dimethylphenyl)-11-isobutyl-2,2-dioxo-12-(3-oxocyclobutyl)-9-oxa-2λ⁶-thia-3,5,12,19-tetrazatricyclo[12.3.1.14,8]nonadeca-1(18),4(19),5,7,14,16-hexaen-13-one (1.51 g, 97%) as off-white solid. ¹H NMR (400 MHz, DMSO-d₆) δ 13.0 (broad s, 1H), 8.47 (s, 1H), 7.86 (s, 1H), 7.59 (s, 2H), 7.19 (s, 1H), 7.07 (d, J=7.7 Hz, 2H), 6.13 (s, 1H), 5.23-5.10 (m, 1H), 4.42-4.29 (m, 1H), 4.28-4.10 (m, 1H), 3.99-3.84 (m, 1H), 3.73-3.57 (m, 2H), 3.36 (d, J=3.5 Hz, 1H), 1.99 (s, 6H), 1.66 (t, J=12.3 Hz, 1H), 1.35-1.20 (m, 2H), 0.97-0.78 (m, 1H), 0.70 (d, J=6.4 Hz, 3H), 0.18 (d, J=6.2 Hz, 3H). ESI-MS m/z calc. 548.20935, found 549.0 (M+1)⁺; Retention time: 1.59 minutes (LC method A).

### Step 2: (11R)-6-(2,6-Dimethylphenyl)-11-isobutyl-12-[3-(3-methoxyazetidin-1-yl)cyclobutyl]-2,2-dioxo-9-oxa-2λ⁶-thia-3,5,12,19-tetrazatricyclo nonadeca-1(18),4(19),5,7,14,16-hexaen-13-one (Compound 65)

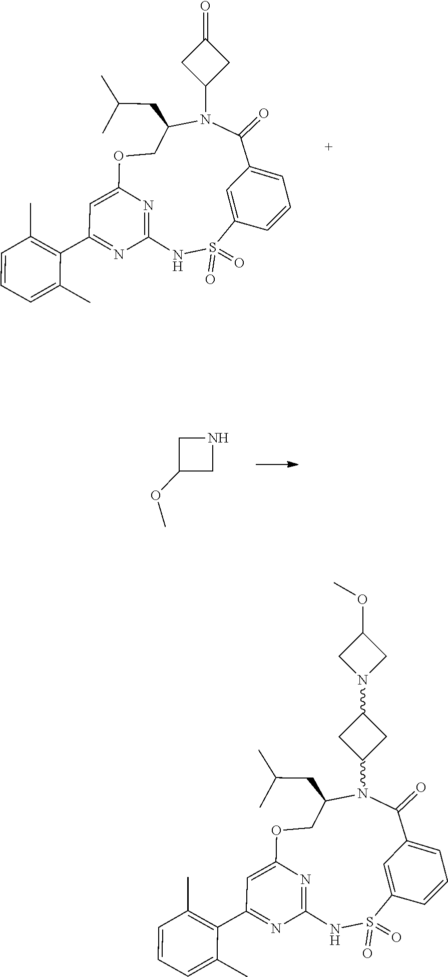

In a 4 mL vial, to a stirred mixture of 3-methoxyazetidine (hydrochloride salt) (10 mg, 0.08092 mmol) in anhydrous dichloromethane (0.4 mL) were added (11R)-6-(2,6-dimethylphenyl)-11-isobutyl-2,2-dioxo-12-(3-oxocyclobutyl)-9-oxa-2λ⁶-thia-3,5,12,19-tetrazatricyclo[12.3.1.14,8]nonadeca-1(18),4(19),5,7,14,16-hexaen-13-one (30 mg, 0.05468 mmol) and sodium triacetoxyborohydride (50 mg, 0.2359 mmol), in that order. The vial was briefly purged with nitrogen and the capped heterogeneous mixture was stirred at ambient temperature for 5 h. Then water (0.1 mL) and methanol (0.3 mL) were added to the reaction and diluted with DMSO (1 mL), microfiltered, and purified by reverse-phase HPLC (Cis column, 5-70% acetonitrile in water, HCl modifier, 15 minute run) to furnish desired (11R)-6-(2,6-dimethylphenyl)-11-isobutyl-12-[3-(3-methoxyazetidin-1-yl)cyclobutyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (15 mg, 32%) as white solid. ESI-MS m/z calc. 619.28284, found 620.2 (M+1)⁺; Retention time: 1.28 minutes; LC method A.

### Step 3: (11R)-12-[3-[3-(Dimethylamino)azetidin-1-yl]cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 66)

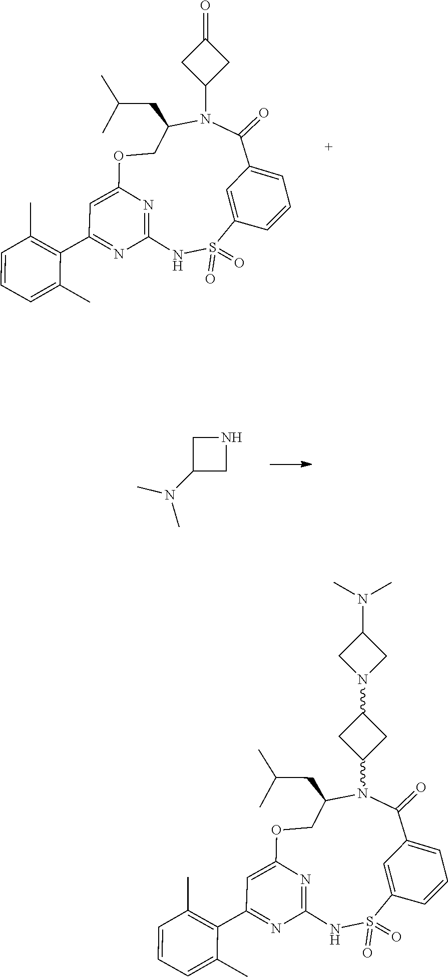

In a 4 mL vial, to a stirred mixture of when N,N-dimethylazetidin-3-amine (Dihydrochloride salt) (13 mg, 0.07511 mmol) in anhydrous dichloromethane (0.4 mL) were added (11R)-6-(2,6-dimethylphenyl)-11-isobutyl-2,2-dioxo-12-(3-oxocyclobutyl)-9-oxa-2λ⁶-thia-3,5,12,19-tetrazatricyclo[12.3.1.14,8]nonadeca-1(18),4(19),5,7,14,16-hexaen-13-one (30 mg, 0.05468 mmol) and sodium triacetoxyborohydride (50 mg, 0.2359 mmol), in that order. The vial was briefly purged with nitrogen and the capped heterogeneous mixture was stirred at ambient temperature for 5 h. Then water (0.1 mL) and methanol (0.3 mL) were added to the reaction and diluted with DMSO (1 mL), microfiltered, and purified by preparative reverse-phase HPLC (Cis column, 5-70% acetonitrile in water, HCl modifier, 15 min run) to furnish (11R)-12-[3-[3-(dimethylamino)azetidin-1-yl]cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Dihydrochloride salt) (19 mg, 46%) as a white solid. ESI-MS m/z calc. 632.31445, found 633.2 (M+1)⁺; Retention time: 1.06 minutes (LC method A).

### Example 51: Preparation of Compound 67

### Step 1: (11R)-6-(2,6-Dimethylphenyl)-12-[3-[3-hydroxypropyl(methyl)amino]cyclobutyl]-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 67)

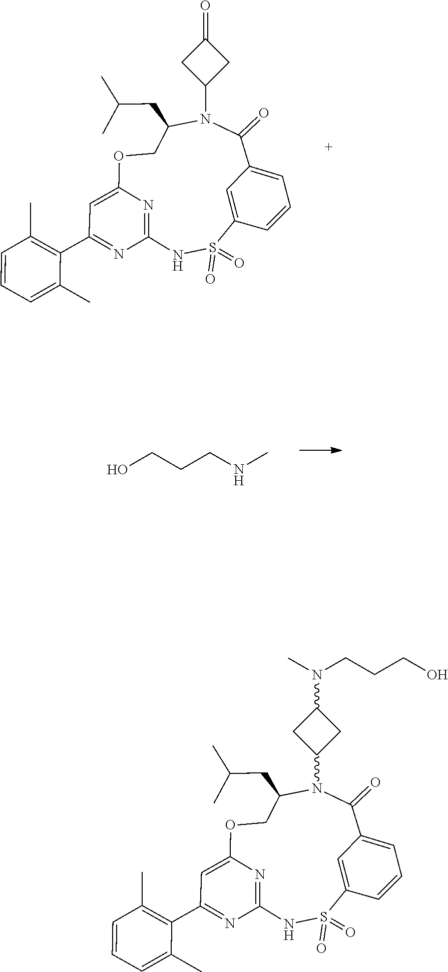

In a 4 mL vial, to a stirred solution of 3-(methylamino)propan-1-ol (8 mg, 0.08975 mmol) in anhydrous dichloromethane (0.4 mL) were added (11R)-6-(2,6-dimethylphenyl)-11-isobutyl-2,2-dioxo-12-(3-oxocyclobutyl)-9-oxa-2λ⁶-thia-3,5,12,19-tetrazatricyclo[12.3.1.14,8]nonadeca-1(18),4(19),5,7,14,16-hexaen-13-one (30 mg, 0.05468 mmol) and sodium triacetoxyborohydride (50 mg, 0.2359 mmol), in that order. The vial was briefly purged with nitrogen and the capped heterogeneous mixture was stirred at ambient temperature for 3 h. Then water (0.1 mL) and methanol (0.3 mL) were added to the reaction and diluted with DMSO (1 mL), microfiltered, and purified by reverse-phase HPLC (Cis column, 5-70% acetonitrile in water, HCl modifier, 15 min run) to furnish (11R)-6-(2,6-dimethylphenyl)-12-[3-[3-hydroxypropyl(methyl)amino]cyclobutyl]-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (17 mg, 46%) as a white solid. ESI-MS m/z calc. 621.29846, found 622.2 (M+1)⁺; Retention time: 1.21 minutes (LC method A).

### Example 52: Preparation of Compound 68

### Step 1: (11R)-6-(2,6-Dimethylphenyl)-11-isobutyl-12-[3-[methyl-[(1R)-2,2,2-trifluoro-1-methyl-ethyl]amino]cyclobutyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 68)

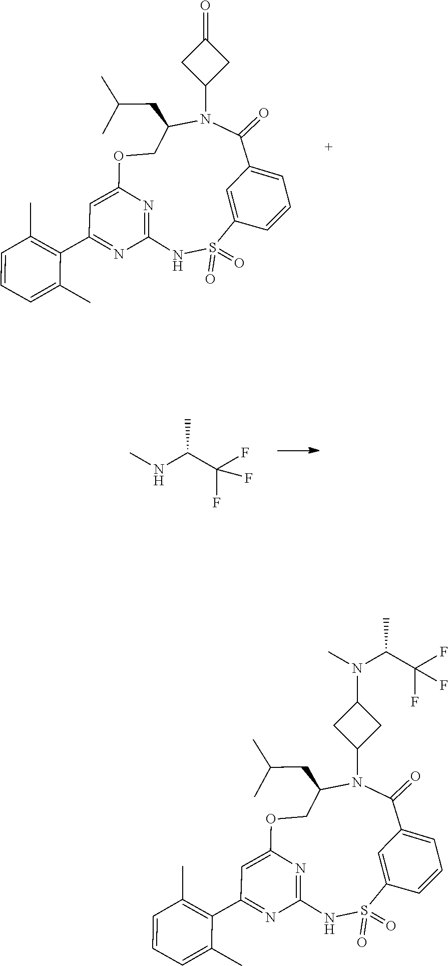

In a 4 mL vial, to a stirred solution of when (2R)-1,1,1-trifluoro-N-methyl-propan-2-amine (hydrochloride salt) (12 mg, 0.07336 mmol) in anhydrous dichloromethane (0.5 mL) were added (11R)-6-(2,6-dimethylphenyl)-11-isobutyl-2,2-dioxo-12-(3-oxocyclobutyl)-9-oxa-2λ⁶-thia-3,5,12,19-tetrazatricyclo[12.3.1.14,8]nonadeca-1(18),4(19),5,7,14,16-hexaen-13-one (25 mg, 0.04557 mmol) and sodium triacetoxyborohydride (40 mg, 0.1887 mmol), in that order. The vial was briefly purged with nitrogen and the capped heterogeneous mixture was stirred at ambient temperature for 3 h. Then water (0.1 mL) and methanol (0.3 mL) were added to the reaction and diluted with DMSO (1 mL), microfiltered, and purified by preparative reverse-phase HPLC (Cis column, 5-70% acetonitrile in water, HCl modifier, 15 min run) to give (11R)-6-(2,6-dimethylphenyl)-11-isobutyl-12-[3-[methyl-[(1R)-2,2,2-trifluoro-1-methyl-ethyl]amino]cyclobutyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (6 mg, 19%) was obtained as white solid. ESI-MS m/z calc. 659.2753, found 660.2 (M+1)⁺; Retention time: 1.63 minutes (LC method A).

### Example 53: Preparation of Compound 69

### Step 1: (11R)-12-[3-[3,3-dimethylbutyl(methyl)amino]cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 69)

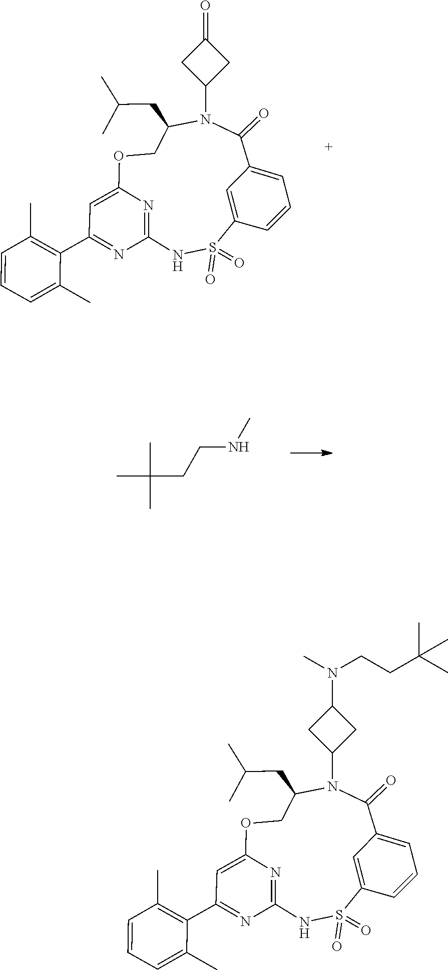

In a 4 mL vial, to a stirred solution of N,3,3-trimethylbutan-1-amine (hydrochloride salt) (12 mg, 0.07912 mmol) in anhydrous dichloromethane (0.5 mL) were added (11R)-6-(2,6-dimethylphenyl)-11-isobutyl-2,2-dioxo-12-(3-oxocyclobutyl)-9-oxa-2λ⁶-thia-3,5,12,19-tetrazatricyclo[12.3.1.14,8]nonadeca-1(18),4(19),5,7,14,16-hexaen-13-one (25 mg, 0.04557 mmol) and sodium triacetoxyborohydride (40 mg, 0.1887 mmol), in that order. The vial was briefly purged with nitrogen and the capped heterogeneous mixture was stirred at ambient temperature for 3 h. Then water (0.1 mL) and methanol (0.3 mL) were added to the reaction and diluted with DMSO (1 mL), microfiltered, and purified by preparative reverse-phase HPLC (Cis column, 5-70% acetonitrile in water, HCl modifier, 15 min run) to give (11R)-12-[3-[3,3-dimethylbutyl(methyl)amino]cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (4 mg, 13%) as white solid. ESI-MS m/z calc. 647.3505, found 648.3 (M+1)⁺; Retention time: 1.52 minutes (LC method A).

### Example 54: Preparation of Compound 70

### Step 1: (11R)-12-[3-[(4,4-Dimethylcyclohexyl)amino]cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 70)

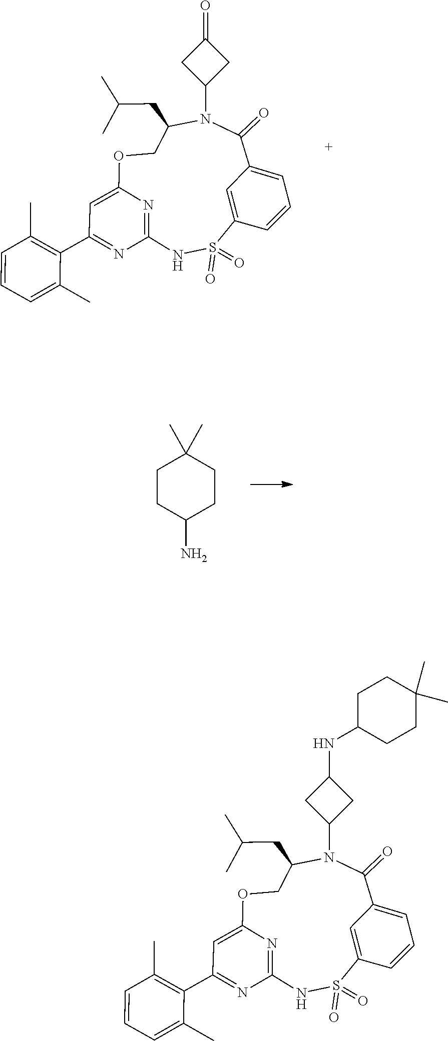

In a 4 mL vial, to a stirred solution of 4,4-dimethylcyclohexanamine (9 mg, 0.07074 mmol) in anhydrous dichloromethane (0.5 mL) were added (11R)-6-(2,6-dimethylphenyl)-11-isobutyl-2,2-dioxo-12-(3-oxocyclobutyl)-9-oxa-2λ⁶-thia-3,5,12,19-tetrazatricyclo[12.3.1.14,8]nonadeca-1(18),4(19),5,7,14,16-hexaen-13-one (25 mg, 0.04557 mmol) and sodium triacetoxyborohydride (40 mg, 0.1887 mmol), in that order. The vial was briefly purged with nitrogen and the capped heterogeneous mixture was stirred at ambient temperature for 3 h. Then water (0.1 mL) and methanol (0.3 mL) were added to the reaction and diluted with DMSO (1 mL), microfiltered, and purified by preparative reverse-phase HPLC (Cis column, 5-70% acetonitrile in water, HCl modifier, 15 min run) to furnish (11R)-12-[3-[(4,4-dimethylcyclohexyl)amino]cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (6 mg, 19%) as white solid. ESI-MS m/z calc. 659.3505, found 660.2 (M+1)⁺; Retention time: 1.57 minutes (LC method A).

### Example 55: Preparation of Compound 71

### Step 1: (11R)-6-(2,6-dimethylphenyl)-11-isobutyl-2,2-dioxo-12-[3-(tetrahydropyran-4-ylamino)cyclobutyl]-9-oxa-2λ⁶-thia-3,5,12,19-tetrazatricyclo[12.3.1.14,8]nonadeca-1(18),4(19),5,7,14,16-hexaen-13-one (Compound 71)

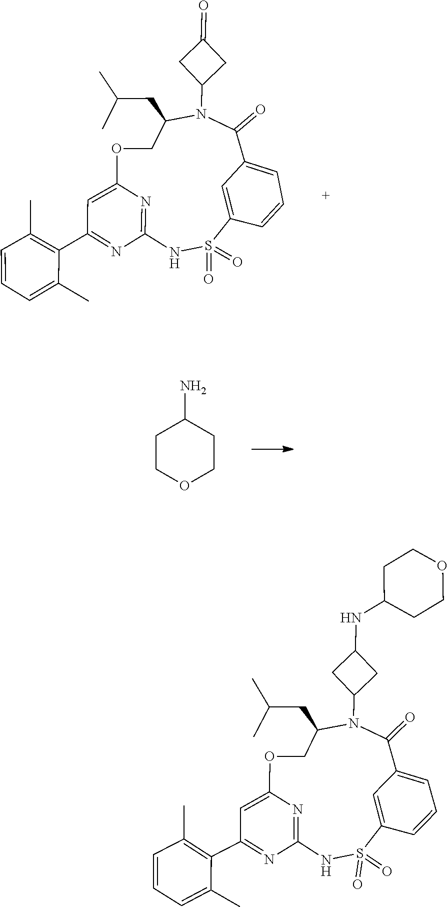 [SMILES: Cc1cccc(C)c1-c1cc2nc(n1)NS(=O)(=O)c1cccc(c1)C(=O)N(C1CC(=O)C1)[C@H](CC(C)C)CO2 ; NC1CCOCC1 ; Cc1cccc(C)c1-c1cc2nc(n1)NS(=O)(=O)c1cccc(c1)C(=O)N(C1CC(NC3CCOCC3)C1)[C@H](CC(C)C)CO2]

In a 4 mL vial, to a stirred solution of tetrahydropyran-4-amine (8 mg, 0.07909 mmol) in anhydrous dichloromethane (0.5 mL) were added (11R)-6-(2,6-dimethylphenyl)-11-isobutyl-2,2-dioxo-12-(3-oxocyclobutyl)-9-oxa-2λ⁶-thia-3,5,12,19-tetrazatricyclo[12.3.1.14,8]nonadeca-1(18),4(19),5,7,14,16-hexaen-13-one (25 mg, 0.04557 mmol) and sodium triacetoxyborohydride (40 mg, 0.1887 mmol), in that order. The vial was briefly purged with nitrogen and the capped heterogeneous mixture was stirred at ambient temperature for 3 h. Then water (0.1 mL) and methanol (0.3 mL) were added to the reaction and diluted with DMSO (1 mL), microfiltered, and purified by preparative reverse-phase HPLC (Cis column, 5-70% acetonitrile in water, HCl modifier, 15 min run) to furnish (11R)-6-(2,6-dimethylphenyl)-11-isobutyl-2,2-dioxo-12-[3-(tetrahydropyran-4-ylamino)cyclobutyl]-9-oxa-2λ⁶-thia-3,5,12,19-tetrazatricyclo[12.3.1.14,8]nonadeca-1(18),4(19),5,7,14,16-hexaen-13-one (hydrochloride salt) (4 mg, 13%) was obtained as white solid. ESI-MS m/z calc. 633.29846, found 634.2 (M+1)⁺; Retention time: 1.25 minutes (LC method A).

### Example 56: Preparation of Compound 72

### Step 1: (11R)-6-(2,6-dimethylphenyl)-12-[3-(1,1-dioxo-1,4-thiazinan-4-yl)cyclobutyl]-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 72)

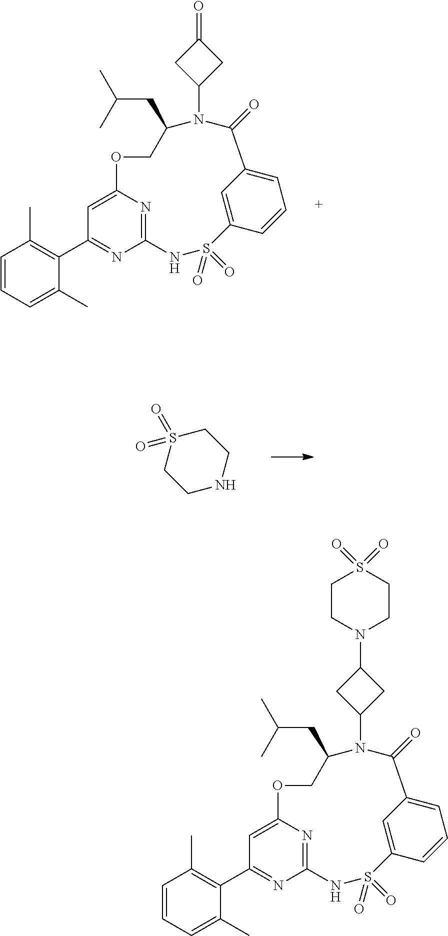 [SMILES: Cc1cccc(C)c1-c1cc2nc(n1)NS(=O)(=O)c1cccc(c1)C(=O)N(C1CC(=O)C1)[C@H](CC(C)C)CO2 ; O=S1(=O)CCNCC1 ; Cc1cccc(C)c1-c1cc2nc(n1)NS(=O)(=O)c1cccc(c1)C(=O)N(C1CC(N3CCS(=O)(=O)CC3)C1)[C@H](CC(C)C)CO2]

In a 4 mL vial, to a stirred solution of (11R)-6-(2,6-dimethylphenyl)-11-isobutyl-2,2-dioxo-12-(3-oxocyclobutyl)-9-oxa-2λ⁶-thia-3,5,12,19-tetrazatricyclo[12.3.1.14,8]nonadeca-1(18),4(19),5,7,14,16-hexaen-13-one (22 mg, 0.04010 mmol) in anhydrous dichloromethane (0.3 mL) were added 1,4-thiazinane 1,1-dioxide (8 mg, 0.05918 mmol) and sodium triacetoxyborohydride (35 mg, 0.1651 mmol), in that order. The vial was briefly purged with nitrogen and the capped heterogeneous mixture was stirred at ambient temperature for 3 h. Then water (0.1 mL) and methanol (0.3 mL) were added to the reaction and diluted with DMSO (1 mL), microfiltered, and purified by preparative reverse-phase HPLC (Cis column, 5-70% acetonitrile in water, HCl modifier, 15 min run) to give (11R)-6-(2,6-dimethylphenyl)-12-[3-(1,1-dioxo-1,4-thiazinan-4-yl)cyclobutyl]-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (20 mg, 70%) as white solid. ¹H NMR (400 MHz, DMSO-d₆) δ 8.43 (s, 1H), 7.92 (s, 1H), 7.68 (s, 2H), 7.26 (t, J=7.6 Hz, 1H), 7.12 (d, J=7.8 Hz, 2H), 6.39 (s, 1H), 5.18 (d, J=10.3 Hz, 1H), 4.36 (t, J=11.1 Hz, 1H), 3.78-3.66 (m, 2H), 3.06-2.88 (m, 4H), 2.49-2.42 (m, 4H), 2.19-1.79 (m, 8H), 1.66 (t, J=12.5 Hz, 2H), 1.40-1.22 (m, 2H), 1.17 (t, J=11.1 Hz, 2H), 0.76 (d, J=6.5 Hz, 3H), 0.23 (d, J=6.3 Hz, 3H). ESI-MS m/z calc. 667.2498, found 668.2 (M+1)⁺; Retention time: 1.29 minutes (LC method A).

### Example 57: Preparation of Compound 73

### Step 1: (11R)-12-[3-(1-Bicyclo[1.1.1]pentanylamino)cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 73)

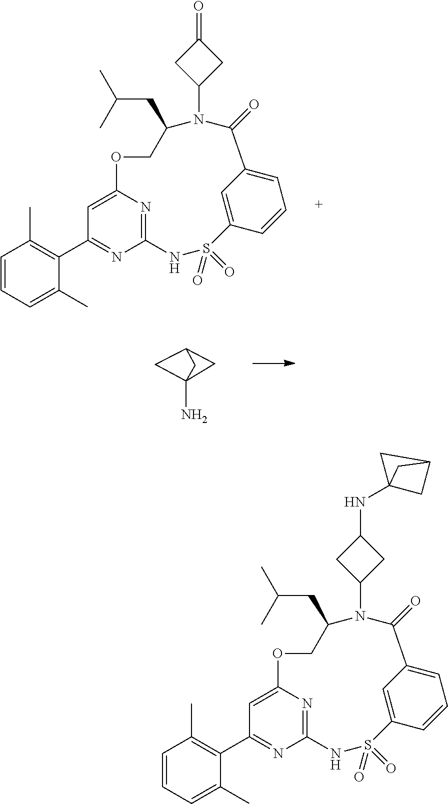 [SMILES: Cc1cccc(C)c1-c1cc2nc(n1)NS(=O)(=O)c1cccc(c1)C(=O)N(C1CC(=O)C1)[C@H](CC(C)C)CO2 ; NC12CC(C1)C2 ; Cc1cccc(C)c1-c1cc2nc(n1)NS(=O)(=O)c1cccc(c1)C(=O)N(C1CC(NC34CC(C3)C4)C1)[C@H](CC(C)C)CO2]

In a 4 mL vial, to a stirred solution of (11R)-6-(2,6-dimethylphenyl)-11-isobutyl-2,2-dioxo-12-(3-oxocyclobutyl)-9-oxa-2λ⁶-thia-3,5,12,19-tetrazatricyclo[12.3.1.14,8]nonadeca-1(18),4(19),5,7,14,16-hexaen-13-one (22 mg, 0.04010 mmol) in anhydrous dichloromethane (0.3 mL) were added bicyclo[1.1.1]pentan-1-amine (hydrochloride salt) (7 mg, 0.05853 mmol) and sodium triacetoxyborohydride (35 mg, 0.1651 mmol), in that order. The vial was briefly purged with nitrogen and the capped heterogeneous mixture was stirred at ambient temperature for 3 h. Then water (0.1 mL) and methanol (0.3 mL) were added to the reaction and diluted with DMSO (1 mL), microfiltered, and purified by preparative reverse-phase HPLC (Cis column, 5-70% acetonitrile in water, HCl modifier, 15 min run) to give (11R)-12-[3-(1-bicyclo[1.1.1]pentanylamino)cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (18 mg, 66%) was obtained as white solid. ESI-MS m/z calc. 615.2879, found 616.2 (M+1)⁺; Retention time: 1.34 minutes; ¹H NMR (400 MHz, DMSO-d₆) δ 13.07 (s, 1H), 9.76 (s, 2H), 8.47 (s, 1H), 7.93 (s, 1H), 7.70 (s, 2H), 7.27 (t, J=7.7 Hz, 1H), 7.13 (d, J=7.6 Hz, 2H), 6.39 (s, 1H), 5.20 (dd, J=10.9, 4.1 Hz, 1H), 4.29 (t, J=11.2 Hz, 1H), 3.89 (p, J=8.5 Hz, 1H), 3.79-3.67 (m, 1H), 3.43 (t, J=7.8 Hz, 1H), 3.10-2.97 (m, 2H), 2.68 (s, 1H), 2.61 (t, J=8.3 Hz, 2H), 2.18-2.00 (m, 9H), 1.97 (s, 3H), 1.63 (t, J=12.0 Hz, 1H), 1.35-1.13 (m, 2H), 0.75 (d, J=6.4 Hz, 3H), 0.25 (d, J=6.1 Hz, 3H). ESI-MS m/z calc. 615.2879, found 616.2 (M+1)⁺; Retention time: 1.34 minutes (LC method A).

### Example 58: Preparation of Compound 74

### Step 1: Ethyl 3-nitro-1H-pyrazole-5-carboxylate

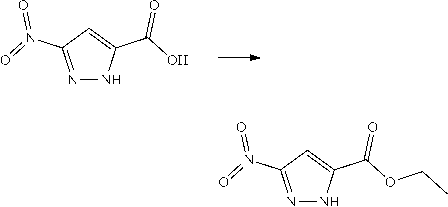

To a solution 3-nitro-1H-pyrazole-5-carboxylic acid (25 g, 159.15 mmol) in EtOH (250 mL) at rt was added acetyl chloride (37.536 g, 34 mL, 478.18 mmol) slowly. The mixture was stirred at reflux for 4 h. The mixture was concentrated and co-evaporated with EtOH (100 mL) and 1,4-dioxane (50 mL) to give ethyl 3-nitro-1H-pyrazole-5-carboxylate (30 g, 100%) as off-white solid. ESI-MS m/z calc. 185.0437, found 186.1 (M+1)⁺; Retention time: 1.58 minutes. ¹H NMR (300 MHz, CDCl₃) δ 7.41 (s, 1H), 4.47 (q, J=7.0 Hz, 2H), 1.43 (t, J=7.0 Hz, 3H), 1.25 (s, 1H), LC method K.

### Step 2: Ethyl 2-methyl-5-nitro-pyrazole-3-carboxylate

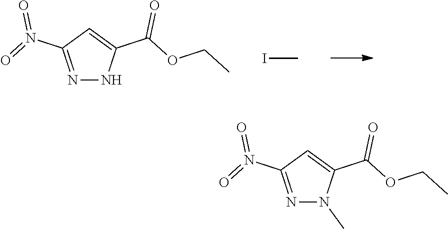

To a solution of ethyl 3-nitro-1H-pyrazole-5-carboxylate (29.6 g, 154.61 mmol) in DMF (200 mL) at 0° C. was added potassium carbonate (44.2 g, 319.81 mmol) and iodomethane (34.200 g, 15 mL, 240.95 mmol) dropwise over 15 min. The mixture was stirred at rt overnight. The mixture was cooled with ice-water bath and cold water (600 mL) was added. The precipitate was collected by filtration and washed with cold water. The resulting precipitate was dissolved in EtOAc (200 mL), dried over sodium sulfate, filtered and concentrated to dryness to give ethyl 2-methyl-5-nitro-pyrazole-3-carboxylate (24.55 g, 78%) as a pale orange solid. ¹H NMR (400 MHz, CDCl₃) δ 7.41 (s, 1H), 4.42 (q, J=7.3 Hz, 2H), 4.29 (s, 3H), 1.42 (t, J=7.2 Hz, 3H). ESI-MS m/z calc. 199.0593, found 200.2 (M+1)⁺; Retention time: 1.66 minutes (LC method X).

### Step 3: Ethyl 5-amino-2-methyl-pyrazole-3-carboxylate

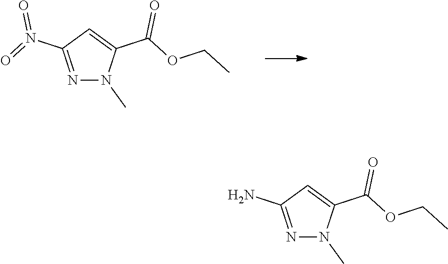

A mixture of ethyl 2-methyl-5-nitro-pyrazole-3-carboxylate (24.74 g, 124.22 mmol), 10% Palladium on carbon 50% wet (8 g, 3.7587 mmol) and MeOH (250 mL) was hydrogenated under hydrogen (balloon) for 24 h. The mixture was filtered through diatomaceous earth and washed with EtOAc. The filtrate was concentrated to give ethyl 5-amino-2-methyl-pyrazole-3-carboxylate (20.88 g, 99%) as white solid. ESI-MS m/z calc. 169.0851, found 170.1 (M+1)⁺; Retention time: 1.33 minutes. ¹H NMR (300 MHz, CDCl₃) δ 6.13 (s, 1H), 4.30 (q, J=7.1 Hz, 2H), 3.99 (s, 3H), 3.62 (br. s., 2H), 1.35 (t, J=7.0 Hz, 3H). LC method K.

### Step 4: Ethyl 5-chlorosulfonyl-2-methyl-pyrazole-3-carboxylate

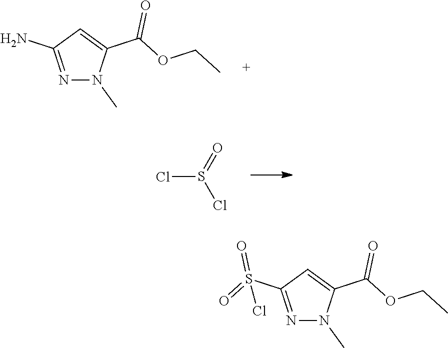

A 500-mL three-neck flask was charged with water (200 mL) and cooled with ice-water bath. Thionyl chloride (66.055 g, 40.5 mL, 555.22 mmol) was added dropwise over 20 minutes. The mixture was stirred at room temperature for 2 hours. Copper(I) chloride (800 mg, 8.0809 mmol) was added and the mixture was cooled to −5° C. Another 250-mL flask was charged with hydrochloric acid solution (37 wt %) (120 mL of 12 M, 1.4400 mol) and ethyl 5-amino-2-methyl-pyrazole-3-carboxylate (20.23 g, 107.38 mmol) was added. The mixture was cooled to −5° C. and a solution of sodium nitrite (9.26 g, 134.21 mmol) in water (50 mL) was added dropwise over 30 minutes, keeping the inner temperature between −6° C. and −3° C. The mixture was stirred at −5° C. for 30 minutes, cooled to −10° C., and slowly canulated (˜25 minutes) to the first solution. The resulting mixture was stirred at 0-5° C. (ice-water bath) for 90 minutes. More copper(I) chloride (270 mg, 2.7273 mmol) was added and the resulting mixture was stirred at 0-5° C. (ice-water bath) for 1 hour. The mixture was extracted with ethyl acetate (2×200 mL), the organic layer was dried with sodium sulfate, filtered and concentrated to dryness. The crude material was purified in two equal batches by Flash chromatography on silica gel (120 g silica gel+100 g) eluted with 0% to 20% ethyl acetate in heptane to afford ethyl 5-chlorosulfonyl-2-methyl-pyrazole-3-carboxylate (12.1 g, 43%) as a colorless oil. 1H NMR (400 MHz, CDCl₃) δ 7.40 (s, 1H), 4.42 (q, J=7.1 Hz, 2H), 4.33 (s, 3H), 1.42 (t, J=7.1 Hz, 3H). ESI-MS m/z calc. 251.9972, found 253.0 (M+1)⁺; Retention time: 4.03 minutes (LC method Y).

### Step 5: Ethyl 5-[[4-chloro-6-(2,6-dimethylphenyl)pyrimidin-2-yl]sulfamoyl]-2-methyl-pyrazole-3-carboxylate

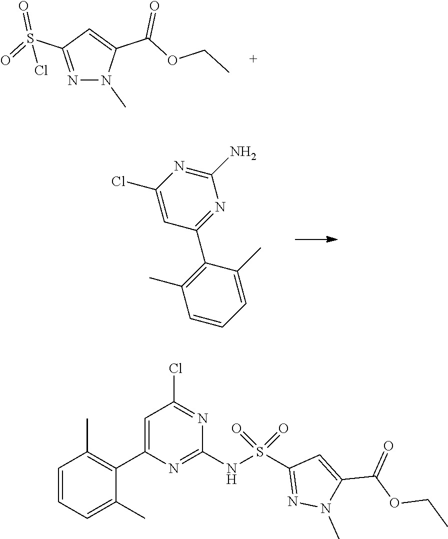 [SMILES: CCOC(=O)c1cc(S(=O)(=O)Cl)nn1C ; Cc1cccc(C)c1-c1cc(Cl)nc(N)n1 ; CCOC(=O)c1cc(S(=O)(=O)Nc2nc(Cl)cc(-c3c(C)cccc3C)n2)nn1C]

To a solution of 4-chloro-6-(2,6-dimethylphenyl)pyrimidin-2-amine (4.8 g, 20.539 mmol) in THE (140 mL) at 0° C. was added a solution of ethyl 5-chlorosulfonyl-2-methyl-pyrazole-3-carboxylate (6.13 g, 23.217 mmol), followed by sodium tert-amoxide in toluene (13.9 mL of 40% w/v, 50.486 mmol) dropwise. The mixture was stirred at rt for 1.5 h. The mixture was slowly poured into a 1 N aqueous HCl (50 mL) at 0° C. The mixture was diluted with water 100 mL and extracted with EtOAc (3×100 mL). The combined organic layers were dried over sodium sulfate filtered and concentrated to dryness. The crude material was purified by flash chromatography on silica gel (330 g) eluted with 5% to 30% ethyl acetate in heptane and the 100% ethyl acetate to give ethyl 5-[[4-chloro-6-(2,6-dimethylphenyl)pyrimidin-2-yl]sulfamoyl]-2-methyl-pyrazole-3-carboxylate (6.77 g, 72%) as white solid. ¹H NMR (400 MHz, CDCl₃) δ 7.95 (br. s., 1H), 7.49 (s, 1H), 7.23 (t, J=8.1 Hz, 1H), 7.09 (d, J=7.6 Hz, 2H), 6.94 (s, 1H), 4.36 (q, J=7.3 Hz, 2H), 4.24 (s, 3H), 2.03 (s, 6H), 1.37 (t, J=7.2 Hz, 3H). ESI-MS m/z calc. 449.0925, found 450.2 (M+1)⁺; Retention time: 4.42 minutes (LC method (LC method A).

### Step 6: 5-[[4-Chloro-6-(2,6-dimethylphenyl)pyrimidin-2-yl]sulfamoyl]-2-methyl-pyrazole-3-carboxylic acid

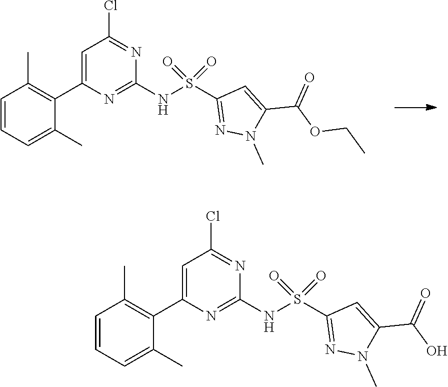 [SMILES: CCOC(=O)c1cc(S(=O)(=O)Nc2nc(Cl)cc(-c3c(C)cccc3C)n2)nn1C ; Cc1cccc(C)c1-c1cc(Cl)nc(NS(=O)(=O)c2cc(C(=O)O)n(C)n2)n1]

To a solution of ethyl 5-[[4-chloro-6-(2,6-dimethylphenyl)pyrimidin-2-yl]sulfamoyl]-2-methyl-pyrazole-3-carboxylate (7.62 g, 16.598 mmol) in THE (220 mL) at 0° C. was added a solution of NaOH (2.7 g, 67.505 mmol) in water (50 mL) and the mixture was stirred for 20 minutes. The mixture was concentrated to remove THF, diluted with water (100 mL) and washed with ethyl acetate (2×100 mL); the combined organic layers were discarded. The aqueous layer was cooled to 0° C., acidified to pH 3-4 with 1N aqueous HCl and extracted with ethyl acetate (3×150 mL). The combined organic layers were dried over sodium sulfate, filtered and concentrated to dryness to give 5-[[4-chloro-6-(2,6-dimethylphenyl)pyrimidin-2-yl]sulfamoyl]-2-methyl-pyrazole-3-carboxylic acid (7.04 g, 99%) as a white solid. ¹H NMR (400 MHz, DMSO-d₆) δ 13.83 (br. s., 1H), 12.48 (br. s., 1H), 7.33 (s, 1H), 7.24 (t, J=8.1 Hz, 1H), 7.13-7.08 (m, 3H), 4.09 (s, 3H), 1.90 (s, 6H). ESI-MS m/z calc. 421.0612, found 422.1 (M+1)⁺; Retention time: 4.04 minutes (LC method Y).

### Step 7: 5-[[4-[(2R)-2-Amino-4-methyl-pentoxy]-6-(2,6-dimethylphenyl)pyrimidin-2-yl]sulfamoyl]-2-methyl-pyrazole-3-carboxylic acid

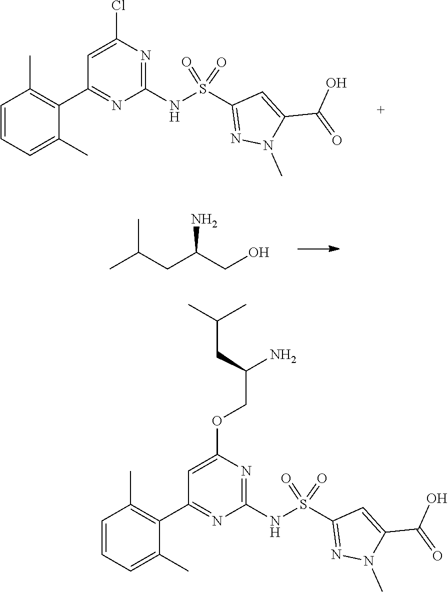

5-[[4-chloro-6-(2,6-dimethylphenyl)pyrimidin-2-yl]sulfamoyl]-2-methyl-pyrazole-3-carboxylic acid (250 mg, 0.5926 mmol) and (2R)-2-amino-4-methyl-pentan-1-ol (100 μL) were combined in THE (1.3 mL) and stirred until the reaction mixture became homogeneous. Sodium tert-butoxide (250 mg, 2.601 mmol) was added and the reaction mixture became warm to the touch and was stirred for 10 minutes without external heating. The reaction mixture was then partitioned between 1M HCl and ethyl acetate. The layers were separated and the aqueous was extracted an additional 3×with ethyl acetate. A substantial amount of product appeared to remain in the aqueous layer, so it was diluted with brine and extracted an additional 5×with ethyl acetate. The combined organics were dried over sodium sulfate and concentrated to give as an off-white solid, which was used in the next step without additional purification. 5-[[4-[(2R)-2-amino-4-methyl-pentoxy]-6-(2,6-dimethylphenyl)pyrimidin-2-yl]sulfamoyl]-2-methyl-pyrazole-3-carboxylic acid (hydrochloride salt) (317 mg, 99%) ESI-MS m/z calc. 502.19983, found 503.3 (M+1)⁺; Retention time: 0.43 minutes (LC method D).

### Step 8: (10R)-15-(2,6-Dimethylphenyl)-10-isobutyl-6-methyl-3,3-dioxo-9-spiro[2.3]hexan-5-yl-12-oxa-3λ⁶-thia-2,5,6,9,16,17-hexazatricyclo[11.3.1.14,7]octadeca-1(17),4,7(18),13,15-pentaen-8-one (Compound 74)

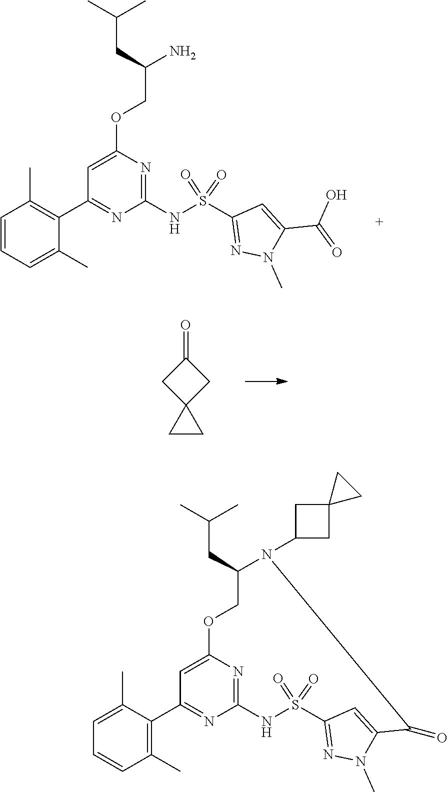 [SMILES: Cc1cccc(C)c1-c1cc(OC[C@H](N)CC(C)C)nc(NS(=O)(=O)c2cc(C(=O)O)n(C)n2)n1 ; O=C1CC2(CC2)C1 ; Cc1cccc(C)c1-c1cc2nc(n1)NS(=O)(=O)c1cc(n(C)n1)C(=O)N(C1CC3(CC3)C1)[C@H](CC(C)C)CO2]

5-[[4-[(2R)-2-amino-4-methyl-pentoxy]-6-(2,6-dimethylphenyl)pyrimidin-2-yl]sulfamoyl]-2-methyl-pyrazole-3-carboxylic acid (hydrochloride salt) (40 mg, 0.07420 mmol) was combined with the spiro[2.3]hexan-5-one (approximately 10.70 mg, 0.1113 mmol) in DCM (0.3 mL), and sodium triacetoxyborohydride (approximately 47.18 mg, 0.2226 mmol) was added. The reaction was stirred for 1 hour at room temperature, then additional sodium triacetoxyborohydride (approximately 47.18 mg, 0.2226 mmol) was added. After an additional 2 hours at room temperature the reaction mixtures were partitioned between 1M HCl and ethyl acetate. The layers were separated and the aqueous was extracted an additional 4×with ethyl acetate. The combined organics were washed with brine, dried over sodium sulfate and concentrated to give crude reductive amination product, which was used in the next step without further purification. The crude material was dissolved in DMF (5 mL) and added at a rapid dropwise to a stirring solution of HATU (approximately 56.43 mg, 0.1484 mmol) and DIPEA (approximately 57.54 mg, 77.55 μL, 0.4452 mmol) in DMF (10 mL). The reaction mixture was stirred for 6 hours at room temperature. The reaction mixture was then partitioned between 1M HCl and ethyl acetate. The layers were separated and the aqueous was extracted an additional 3×with ethyl acetate. The combined organics were washed with brine, dried over sodium sulfate and concentrated. The resulting crude material was purified by reverse phase HPLC (1-99% ACN in water, HCl modifier) to give (10R)-15-(2,6-dimethylphenyl)-10-isobutyl-6-methyl-3,3-dioxo-9-spiro[2.3]hexan-5-yl-12-oxa-3λ⁶-thia-2,5,6,9,16,17-hexazatricyclo[11.3.1.14,7]octadeca-1(17),4,7(18),13,15-pentaen-8-one (4.2 mg, 10%). ESI-MS m/z calc. 564.2519, found 565.6 (M+1)⁺; Retention time: 1.94 minutes; LC method A.

### Example 59: Preparation of Compound 75

### Step 1: 3-[[4-[(2R)-2-[[2-(tert-Butoxycarbonylamino)spiro[3.3]heptan-6-yl]amino]-5,5,5-trifluoro-4,4-dimethyl-pentoxy]-6-(2,6-dimethylphenyl)pyrimidin-2-yl]sulfamoyl]benzoic acid

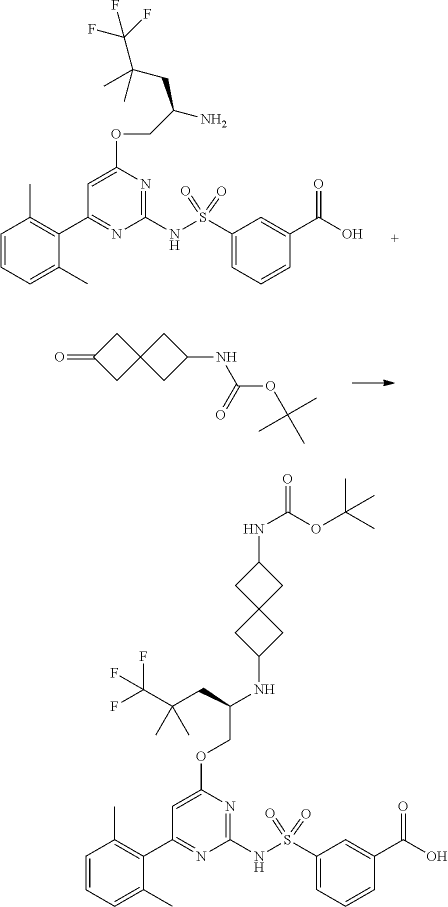

A 20 mL vial was charged with 3-[[4-[(2R)-2-amino-5,5,5-trifluoro-4,4-dimethyl-pentoxy]-6-(2,6-dimethylphenyl)pyrimidin-2-yl]sulfamoyl]benzoic acid (hydrochloride salt) (655 mg, 1.010 mmol), tert-butyl N-(2-oxospiro[3.3]heptan-6-yl)carbamate (280 mg, 1.243 mmol), anhydrous DCM (2 mL) and sodium triacetoxyborohydride (Sodium salt) (710 mg, 3.350 mmol). The vial was briefly purged with nitrogen and the mixture was stirred at rt for 3.5 hours. The mixture was treated with DCM (40 mL), 1N aqueous HCl and brine (total 30 mL) resulting in an aqueous phase and a thick dense gel. The gel was separated, and the aqueous phase was further extracted with ethyl acetate (2×20 mL-no product detected in the aqueous phase). Mixing the ethylacetate and the gel resulted in two phases that were easily separated. The organic phase was dried over sodium sulfate and the solvents were evaporated. The residue was dissolved in DMSO (6 mL) and was purified by reverse phase preparative HPLC (C₁₈) using a gradient of acetonitrile in water (1 to 99% over 15 min) and HCl as a modifier. The pure fractions were collected and the organic solvent evaporated. A bit of brine was added to the aqueous phase and the solid that started to precipitate out was extracted with EtOAc. After drying over sodium sulfate, the organic solvent was evaporated. Trituration in EtOAc/hexanes and evaporation of the solvents gave 3-[[4-[(2R)-2-[[2-(tert-butoxycarbonylamino)spiro[3.3]heptan-6-yl]amino]-5,5,5-trifluoro-4,4-dimethyl-pentoxy]-6-(2,6-dimethylphenyl)pyrimidin-2-yl]sulfamoyl]benzoic acid (hydrochloride salt) (436 mg, 53%) as a white solid. ESI-MS m/z calc. 775.32263, found 776.86 (M+1)⁺; Retention time: 1.42 minutes (LC method A).

### Step 2: tert-butyl N-{6-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]spiro[3.3]heptan-2-yl}carbamate, diastereomer 1, and tert-butyl N-{6-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]spiro[3.3]heptan-2-yl}carbamate, diastereomer 2

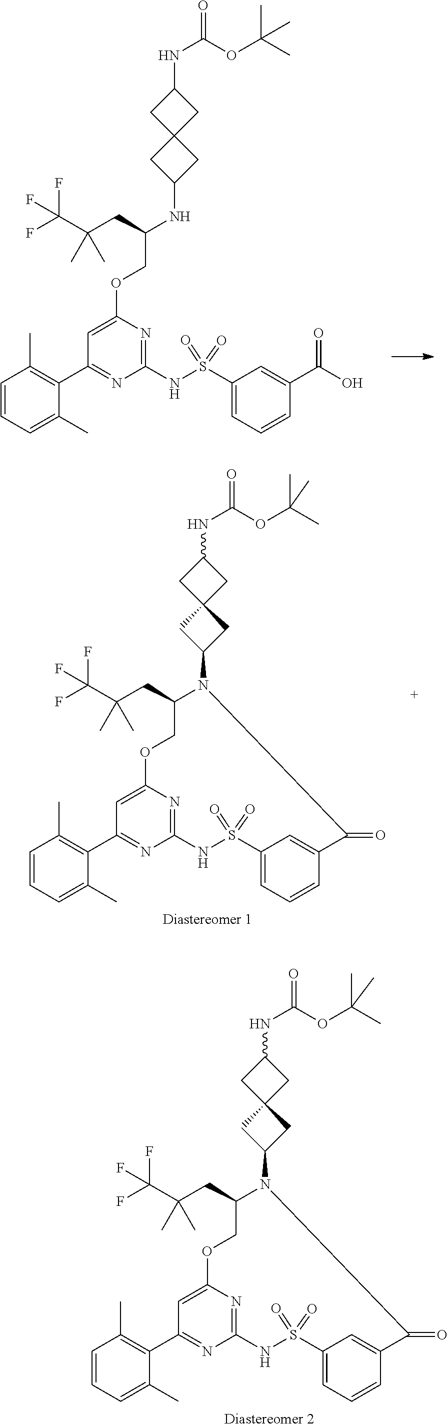

A 20 mL flask was charged under nitrogen with HATU (479 mg, 1.260 mmol), anhydrous DMF (30 mL) and DIEA (0.52 mL, 2.985 mmol). A solution of 3-[[4-[(2R)-2-[[2-(tert-butoxycarbonylamino)spiro[3.3]heptan-6-yl]amino]-5,5,5-trifluoro-4,4-dimethyl-pentoxy]-6-(2,6-dimethylphenyl)pyrimidin-2-yl]sulfamoyl]benzoic acid (hydrochloride salt) (436 mg, 0.5367 mmol) in anhydrous DMF (15 mL) was added dropwise through syringe over a period of 5 minutes. The mixture was stirred at room temperature for 41 hours (at 24 hours, approximatively half the cyclization was complete). The mixture was concentrated and diluted with DMSO (3 mL). The solution was microfiltered through a syringe filter disc and purified by reverse phase preparative HPLC (C₁₈) using a gradient of acetonitrile in water (1 to 99% over 15 min) and HCl as a modifier. The pure fractions were collected and Brine and saturated bicarbonate were added. The organic phase was evaporated and the white precipitate was extracted with EtOAc (2×30 mL). After drying over sodium sulfate, evaporation and trituration in DCM/hexanes tert-butyl N-{6-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]spiro[3.3]heptan-2-yl}carbamate (151 mg, 37%) (diastereomeric mixture). was isolated as a white solid. ESI-MS m/z calc. 757.3121, found 758.68 (M+1)⁺; Retention time: 2.09 minutes (hints of peak doubling visible), LC method A.

The two diastereomers were separated by chiral SFC using a phenomenex LUX-4 column (250×21.2 mm), 5 μM, 40° C.; mobile phase: 34% MeOH (no modifier), 66% CO₂, flow: 70 mL/min, concentration: 16 mg/mL in methanol (no modifier), injection volume 500 μL, 220 bar, wavelength: 210 mm. For each isomer, the solvent was evaporated, and the residue triturated in EtOAc/hexanes. Evaporation of the solvents gave the following compounds as a white solid: Diastereomer 1, SFC peak 1: tert-butyl N-{6-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]spiro[3.3]heptan-2-yl}carbamate (46 mg, 23%). ESI-MS m/z calc. 757.3121, found 758.46 (M+1)⁺; Retention time: 2.07 minutes (LC method A); and diastereomer 2, SFC peak 2: tert-butyl N-{6-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]spiro[3.3]heptan-2-yl}carbamate (41 mg, 20%). ESI-MS m/z calc. 757.3121, found 758.42 (M+1)⁺; Retention time: 2.06 minutes (LC method A).

### Step 3: Methyl N-{6-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]spiro[3.3]heptan-2-yl}carbamate, diastereomer 1 (Compound 75)

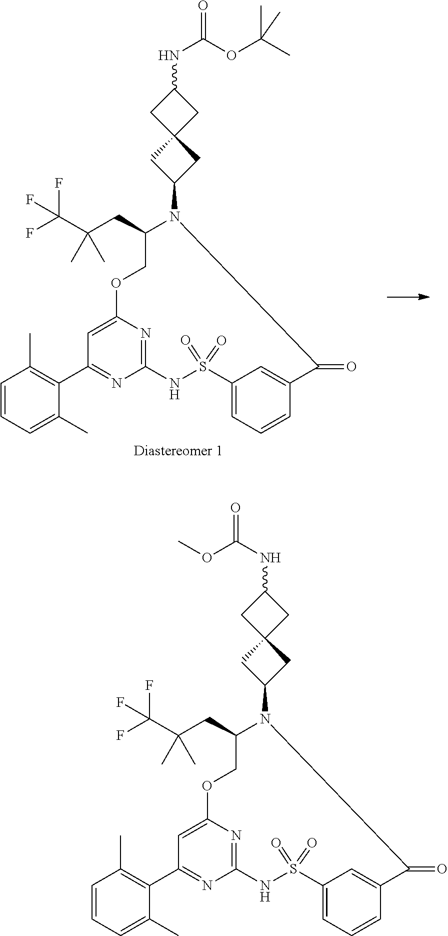

A 100 mL flask containing tert-butyl N-{6-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]spiro[3.3]heptan-2-yl}carbamate (46 mg, 0.06070 mmol) (Diastereomer 1) was treated with DCM (0.6 mL) and HCl (500 μL of 4 M, 2.000 mmol) (4M in dioxane) at room temperature for 1 hour. The volatiles were removed. The residue was treated with DCM/hexanes and the solvents were removed by evaporation. The operation was repeated several times until a white solid was obtained. The solid was treated with anhydrous DCM (1 mL) and DIEA (53 μL, 0.3043 mmol) to give a suspension. Addition of methyl chloroformate (15 μL, 0.1941 mmol) resulted in rapid dissolution of the solids. After 15 min, the volatiles were removed by evaporation and the residue was dissolved in DMSO (1 mL). The solution was microfiltered through a syringe filter disc and purified by reverse phase preparative HPLC (C₁₈) using a gradient of acetonitrile in water (1 to 99% over 15 min) and HCl as a modifier. Genevac evaporation provided a solid that was transferred using EtOAc. Trituration in EtOAc/hexanes and evaporation gave methyl N-{6-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]spiro[3.3]heptan-2-yl}carbamate (25 mg, 57%) as a white solid. ESI-MS m/z calc. 715.26514, found 716.73 (M+1)⁺; Retention time: 1.79 minutes (LC method A). ¹H NMR (400 MHz, DMSO-d₆) δ 13.36-11.78 (broad m, 1H), 8.41 (s, 1H), 7.92 (s, 1H), 7.67 (s, 2H), 7.36 (d, J=7.9 Hz, 1H), 7.30-7.20 (m, 1H), 7.12 (s, 2H), 6.40 (s, 1H), 5.09 (d, J=8.6 Hz, 1H), 4.34 (t, 1H), 4.07-3.70 (m, 3H), 3.50 (s, 3H), 3.04 (m, 2H), 2.38-2.21 (m, 3H), 2.21-2.04 (m, 3H), 2.04-1.81 (m, 7H), 1.80-1.65 (m, 1H), 0.85 (s, 3H overlapped with hexanes signal), 0.61 (s, 3H).

### Example 60: Preparation of Compound 76

### Step 1: Methyl N-{6-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]spiro[3.3]heptan-2-yl}carbamate, diastereomer 2 (Compound 76)

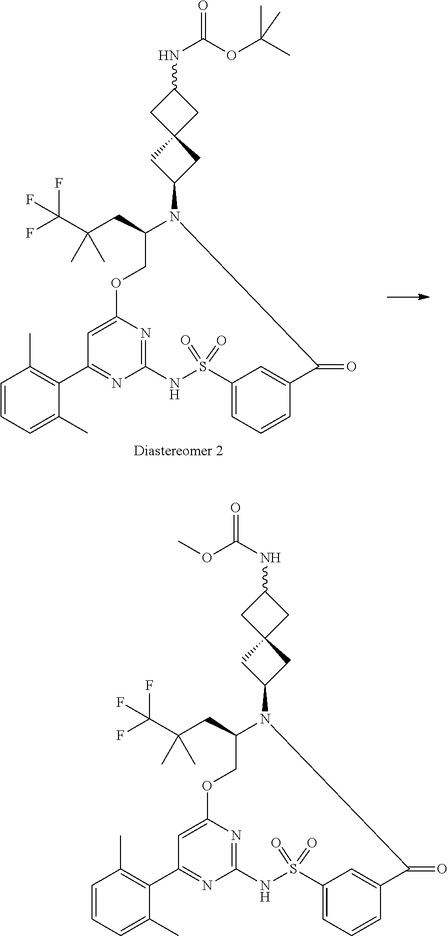

A 100 mL flask containing tert-butyl N-{6-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]spiro[3.3]heptan-2-yl}carbamate (41 mg, 0.05410 mmol) (Diastereomer 2) was treated with DCM (0.6 mL) and HCl (500 μL of 4 M, 2.000 mmol) (4M in dioxane) at room temperature for 1 hour (60% conversion). More HCl (500 μL of 4 M, 2.000 mmol) was added and the reaction was stirred for 45 minutes. The volatiles were removed. The residue was treated with DCM/hexanes and the solvents were removed by evaporation. The operation was repeated several times until a white solid was obtained. The solid was treated with anhydrous DCM (1 mL) and DIEA (53 μL, 0.3043 mmol)) to give a suspension. Addition of methyl chloroformate (15 μL, 0.1941 mmol) resulted in rapid dissolution of the solids. After 8 min, the volatiles were removed by evaporation and the residue was dissolved in DMSO (1 mL). The solution was microfiltered through a syringe filter disc and purified by reverse phase preparative HPLC (C₁₈) using a gradient of acetonitrile in water (1 to 99% over 15 min) and HCl as a modifier. Genevac evaporation provided a 94% pure material (26 mg). It was dissolved in DCM and purified by flash chromatography on silica gel (4 g column) using a gradient of ethyl acetate (10 to 100% over 15 min) in hexanes. The product eluted around 60-70% EA. Evaporation of the solvents gave methyl N-{6-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]spiro[3.3]heptan-2-yl}carbamate (22 mg, 57%). ESI-MS m/z calc. 715.26514, found 716.77 (M+1)⁺; Retention time: 1.78 minutes (LC method A).

### Example 61: Preparation of Compound 77 and Compound 78

### Step 1: tert-Butyl N-[6-[[(1R)-1-(hydroxymethyl)-2-[1-(trifluoromethyl)cyclopropyl]ethyl]amino]spiro[3.3]heptan-2-yl]carbamate

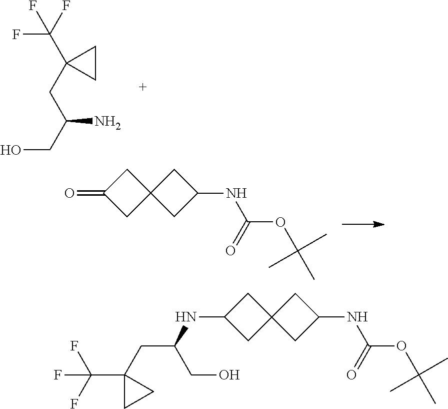

To a slurry of (2R)-2-amino-3-[1-(trifluoromethyl)cyclopropyl]propan-1-ol (hydrochloride salt) (96.2 g, 438.0 mmol) in 1,2-dichloroethane (1,000 mL) was added DIEA (80 mL, 459.3 mmol) and the mixture stirred for 5 min—became homogenous. To the mixture was added tert-butyl N-(2-oxospiro[3.3]heptan-6-yl)carbamate (98.6 g, 437.7 mmol) followed by HOAc (27 mL, 474.8 mmol) and the mixture stirred at ambient temperature for 1 h. To the mixture was added sodium triacetoxyborohydride (106.8 g, 503.9 mmol) and the mixture stirred at ambient temperature (slow exotherm to 30° C. for 30 min, then cooled to ambient temperature). After 3 h, additional sodium triacetoxyborohydride (21.75 g, 102.6 mmol) was added and the reaction was stirred at ambient temperature for 14 h. The mixture was cooled with an ice-water bath and quenched with water (1000 mL) and stirred for 10 min. To the mixture was added HCl (110 mL of 12 M, 1.320 mol) portions followed by isopropyl acetate (1,000 mL). The mixture was basified with NaOH (350 g of 50% w/w, 4.375 mol) and the phases split. The aqueous phase was extracted with isopropyl acetate (1,000 mL). The combined organic phases were washed with 1 L of brine, dried over magnesium sulfate, filtered and concentrated in vacuo. During concentration the product began to precipitate out and was collected using a M frit. The solid was washed twice with 50 mL of MTBE and the combined solids dried in vacuo at 45° C. The solid was diluted with MTBE (9 L) and TsOH (40 g, 232.3 mmol) was added. The creamy, white slurry was stirred for 30 minutes. The precipitate was collected using a M frit. The solid was air dried for 16 h. The solid was slurried with isopropyl acetate (700 mL) and NaOH (500 mL of 2 M, 1.000 mol) until homogenous. The phases were separated, and the organic phase washed with 500 mL of brine. The aqueous phases were extracted with isopropyl acetate (700 mL) and the combined organic phases were dried over magnesium sulfate, filtered and concentrated in vacuo to about 200 mL. The slurry was filtered and a second crop from the filtrate was also collected and were added to the first crop collected. tert-Butyl N-[6-[[(1R)-1-(hydroxymethyl)-2-[1-(trifluoromethyl)cyclopropyl]ethyl]amino]spiro[3.3]heptan-2-yl]carbamate (108.7 g, 63%). ¹H NMR (400 MHz, DMSO-d₆) δ 7.01 (d, J=8.0 Hz, 1H), 4.45 (q, J=5.0 Hz, 1H), 3.78 (h, J=8.3 Hz, 1H), 3.37-3.31 (m, 1H), 3.24 (dt, J=10.8, 5.3 Hz, 1H), 3.10 (p, J=7.5 Hz, 1H), 2.55 (q, J=5.7 Hz, 1H), 2.21 (dt, J=13.4, 6.0 Hz, 2H), 2.04 (p, J=5.6 Hz, 2H), 1.83 (q, J=9.8 Hz, 2H), 1.68-1.43 (m, 5H), 1.35 (s, 9H), 0.86 (s, 2H), 0.77 (d, J=11.1 Hz, 2H). ESI-MS m/z calc. 392.22867, found 393.2 (M+1)⁺; Retention time: 1.66 minutes (LC method A).

### Step 2: 3-[[4-[(2R)-2-[[2-(tert-Butoxycarbonylamino)spiro[3.3]heptan-6-yl]amino]-3-[1-(trifluoromethyl)cyclopropyl]propoxy]-6-(2,6-dimethylphenyl)pyrimidin-2-yl]sulfamoyl]benzoic acid

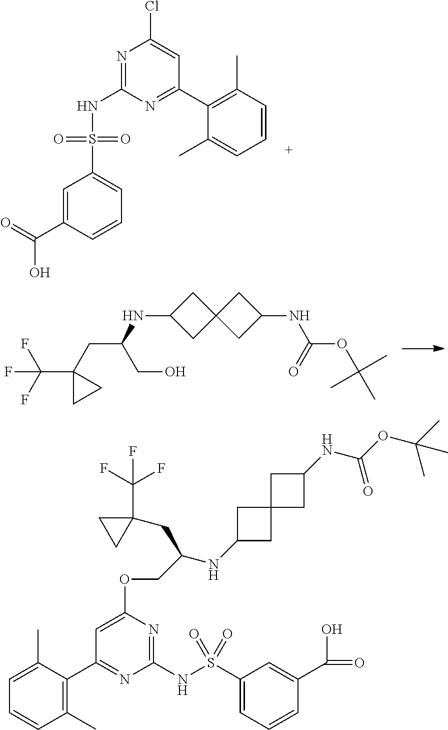

To a solution of tert-butyl N-[6-[[(1R)-1-(hydroxymethyl)-2-[1-(trifluoromethyl)cyclopropyl]ethyl]amino]spiro[3.3]heptan-2-yl]carbamate (108.7 g, 277.0 mmol) and 3-[[4-chloro-6-(2,6-dimethylphenyl)pyrimidin-2-yl]sulfamoyl]benzoic acid (114 g, 268.7 mmol) in 2-MeTHF (1 L) was added sodium tert-butoxide (130 g, 1.353 mol) portion-wise keeping the reaction temperature <40° C. The addition was exothermic, and the reaction temperature was controlled using addition rate of the base. The reaction was stirred for 1 hour at room temperature. The reaction was quenched with the slow addition of HCl (800 mL of 2 M, 1.600 mol) and it was stirred for 5 min. The mixture was transferred to a separatory funnel using 2Me-THF. The aqueous phase was separated, and the organic phase washed with 500 mL of brine. The combined aqueous phases were extracted with 500 mL of 2Me-THF. The combined organic phases were dried over magnesium sulfate, filtered over Celite and the hazy solution concentrated in vacuo. The crude foam was diluted with 2-MeTHF (1 L) and re-dried over magnesium sulfate, filtered over Celite and concentrated in vacuo. 3-[[4-[(2R)-2-[[2-(tert-Butoxycarbonylamino)spiro[3.3]heptan-6-yl]amino]-3-[1-(trifluoromethyl)cyclopropyl]propoxy]-6-(2,6-dimethylphenyl)pyrimidin-2-yl]sulfamoyl]benzoic acid (hydrochloride salt) (217 g, 100%) ESI-MS m/z calc. 773.307, found 774.3 (M+1)⁺; Retention time: 1.21 minutes (LC method A).

### Step 3: tert-butyl N-[2-[(11R)-6-(2,6-Dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]spiro[3.3]heptan-6-yl]carbamate (Compound 77)

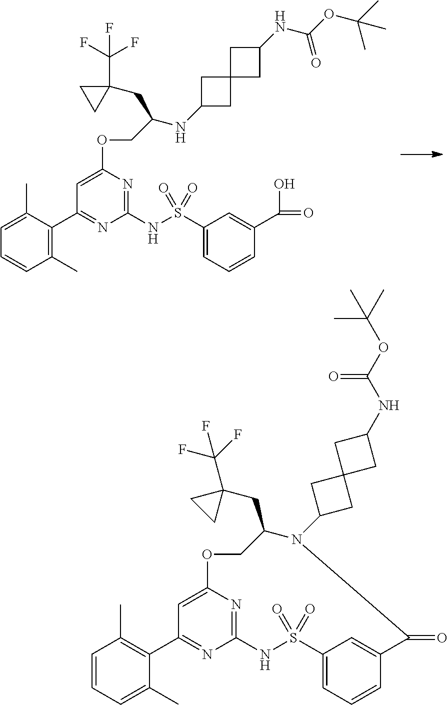 [SMILES: Cc1cccc(C)c1-c1cc(OC[C@@H](CC2(C(F)(F)F)CC2)NC2CC3(CC(NC(=O)OC(C)(C)C)C3)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CC(NC(=O)OC(C)(C)C)C3)C1)[C@H](CC1(C(F)(F)F)CC1)CO2]

To a solution of 3-[[4-[(2R)-2-[[2-(tert-butoxycarbonylamino)spiro[3.3]heptan-6-yl]amino]-3-[1-(trifluoromethyl)cyclopropyl]propoxy]-6-(2,6-dimethylphenyl)pyrimidin-2-yl]sulfamoyl]benzoic acid (hydrochloride salt) (217 g, 267.8 mmol) in DMF (2.7 L) was added DIEA (140 mL, 803.8 mmol) followed by the portionwise addition of HATU (150 g, 394.5 mmol). The mixture was stirred at ambient temperature for 18 h. The mixture was slowly added to a cold solution of HCl (65 mL of 12 M, 780.0 mmol) in water (8 L) over 30 min and the cream colored slurry was stirred at ambient temperature for 10 min. The tan slurry was filtered using a M frit (slow filtration). The precipitate was washed 3 times with 100 mL of water and air dried for 1 h. The wet filter cake was dissolved in iPrOAc (3 L) and the water phase separated. The organic phase was washed with 1 L of brine. The aqueous phases were extracted with 500 mL of iPrOAc. The combined organic phases were dried over magnesium sulfate, filtered over Celite and concentrated in vacuo. The crude product was chromatographed on a 1.5 Kg column eluting with 20-70% EtOAc/hexanes (product eluted at 60% EtOAc). Pure fractions were concentrated. The fractions which contained some impurities were combined and concentrated. The impure fractions were chromatographed on a 750 g column eluting with 30-65% EtOAc/hexanes. The product began to crystallize out during concentration and was dried in vacuo overnight. The Impure product was diluted with 50 mL of EtOAc, seeded and allowed to stand overnight. The slurry was filtered using a M filter funnel and washed 3 times with 1:1 EtOAc/hexanes. This afforded tert-butyl N-[2-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]spiro[3.3]heptan-6-yl]carbamate (152 g, 74%). ¹H NMR (400 MHz, DMSO-d₆) δ 13.03 (s, 1H), 8.33 (d, J=6.0 Hz, 1H), 7.88 (d, J=7.5 Hz, 1H), 7.74-7.55 (m, 2H), 7.26 (t, J=7.7 Hz, 1H), 7.09 (dd, J=23.0, 7.8 Hz, 3H), 6.37 (s, 1H), 5.04 (dt, J=10.5, 4.9 Hz, 1H), 4.33 (td, J=11.5, 5.5 Hz, 1H), 4.06 (s, 1H), 3.79 (tt, J=17.3, 8.4 Hz, 2H), 3.05-2.86 (m, 2H), 2.37 (dt, J=12.3, 6.2 Hz, 1H), 2.29-2.02 (m, 7H), 1.93 (q, J=8.1, 6.7 Hz, 5H), 1.48 (ddd, J=15.7, 9.3, 6.0 Hz, 1H), 1.37 (s, 9H), 0.78 (ddq, J=19.2, 9.5, 4.7, 4.3 Hz, 2H), 0.70-0.50 (m, 2H). ESI-MS m/z calc. 755.29645, found 756.2 (M+1)⁺; Retention time: 2.88 minutes (LC method I).

### Step 4: tert-butyl ((2S,4s,6S)-6-((R)-1⁶-(2,6-dimethylphenyl)-3,3-dioxido-5-oxo-7-((1-(trifluoromethyl)cyclopropyl)methyl)-9-oxa-3-thia-2,6-diaza-1(2,4)-pyrimidina-4(1,3)-benzenacyclononaphane-6-yl)spiro[3.3]heptan-2-yl)carbamate, and tert-butyl ((2R,4r,6R)-6-((R)-1⁶-(2,6-dimethylphenyl)-3,3-dioxido-5-oxo-7-((1-(trifluoromethyl)cyclopropyl)methyl)-9-oxa-3-thia-2,6-diaza-1(2,4)-pyrimidina-4 (1,3)-benzenacyclononaphane-6-yl spiro [3.3]heptan-2-yl)carbamate

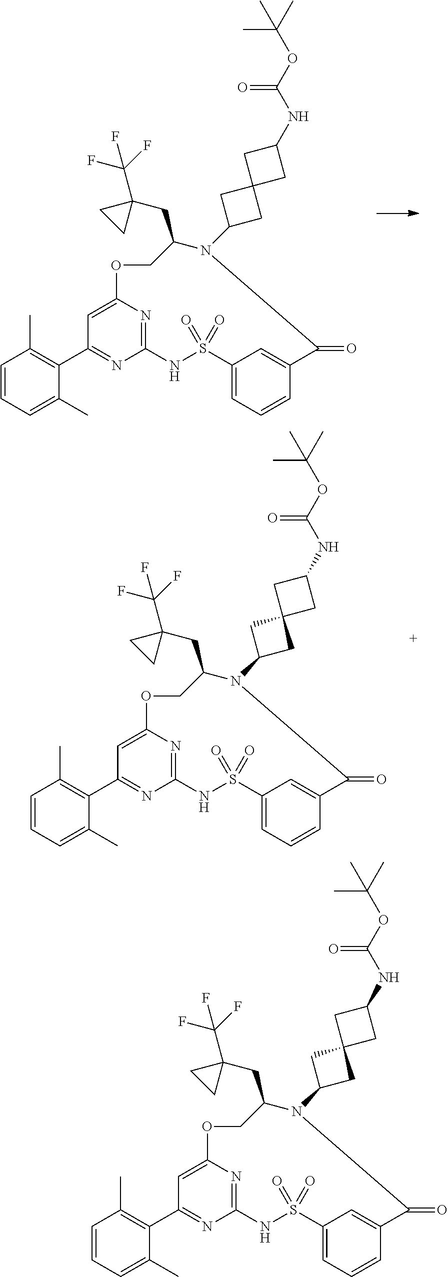

A sample of 150 g of tert-butyl N-[2-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]spiro[3.3]heptan-6-yl]carbamate (150 g, 198.5 mmol) was subjected to chiral SFC separation using a LUX-CEL-4 column (2×25 cm) with a mobile phase of 40% methanol/CO₂ at 70 mL/min. Sample concentration was 20 mg/mL in methanol, with 4 mL injections, outlet pressure of 100 bar, and detection wavelength of 220 nm to give, two peaks:

Peak 1: tert-butyl ((2S,4s,6S)-6-((R)-1⁶-(2,6-dimethylphenyl)-3,3-dioxido-5-oxo-7-((1-(trifluoromethyl)cyclopropyl)methyl)-9-oxa-3-thia-2,6-diaza-1(2,4)-pyrimidina-4(1,3)-benzenacyclononaphane-6-yl)spiro[3.3]heptan-2-yl)carbamate (73 g, 97%) ¹H NMR (400 MHz, DMSO-d₆) δ 13.03 (s, 1H), 8.32 (s, 1H), 7.87 (s, 1H), 7.64 (s, 2H), 7.25 (t, J=7.6 Hz, 1H), 7.15-6.98 (m, 3H), 6.36 (s, 1H), 5.03 (dd, J=10.8, 4.5 Hz, 1H), 4.33 (dt, J=14.3, 7.6 Hz, 1H), 4.03 (d, J=9.6 Hz, 1H), 3.79 (tt, J=17.3, 8.5 Hz, 2H), 3.01 (t, J=9.3 Hz, 1H), 2.91 (t, J=9.9 Hz, 1H), 2.37 (dt, J=12.1, 7.0 Hz, 1H), 2.31-2.00 (m, 7H), 2.00-1.84 (m, 5H), 1.49 (dd, J=16.7, 9.6 Hz, 1H), 1.37 (s, 9H), 0.78 (ddt, J=19.1, 9.9, 4.8 Hz, 2H), 0.70-0.51 (m, 2H). ESI-MS m/z calc. 755.29645, found 756.4 (M+1)⁺; Retention time: 2.84 minutes (LC method I).

Peak 2: tert-butyl ((2R,4r,6R)-6-((R)-16-(2,6-dimethylphenyl)-3,3-dioxido-5-oxo-7-((1-(trifluoromethyl)cyclopropyl)methyl)-9-oxa-3-thia-2,6-diaza-1(2,4)-pyrimidina-4(1,3)-benzenacyclononaphane-6-yl)spiro[3.3]heptan-2-yl)carbamate (63 g, 84%)¹H NMR (400 MHz, DMSO-d₆) δ 13.02 (s, 1H), 8.33 (s, 1H), 7.87 (d, J=7.1 Hz, 1H), 7.63 (d, J=11.8 Hz, 2H), 7.25 (t, J=7.7 Hz, 1H), 7.09 (dd, J=21.2, 7.8 Hz, 3H), 6.36 (s, 1H), 5.04 (dd, J=10.9, 4.4 Hz, 1H), 4.50-4.20 (m, 1H), 4.03 (d, J=13.0 Hz, 1H), 3.79 (tt, J=17.4, 8.5 Hz, 2H), 2.94 (dt, J=25.0, 9.7 Hz, 2H), 2.38 (dt, J=11.6, 6.2 Hz, 1H), 2.32-2.01 (m, 7H), 1.93 (q, J=8.7 Hz, 5H), 1.47 (dd, J=16.5, 9.4 Hz, 1H), 1.37 (s, 9H), 0.78 (tdd, J=15.1, 10.1, 4.8 Hz, 2H), 0.68-0.50 (m, 2H). ESI-MS m/z calc. 755.29645, found 756.4 (M+1)⁺; Retention time: 2.82 minutes (LC method I).

### Step 5: (R)-6-((2S,4s,6S)-6-Aminospiro[3.3]heptan-2-yl)-1⁶-(2,6-dimethylphenyl)-7-((1-(trifluoromethyl)cyclopropyl)methyl)-9-oxa-3-thia-2,6-diaza-1(2,4)-pyrimidina-4(1,3)-henzenacyclononaphan-5-one 3,3-dioxide

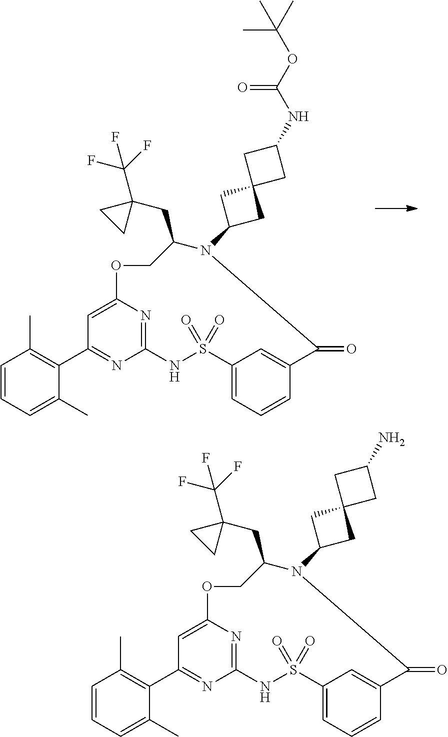

To a solution of tert-butyl ((2S,4s,6S)-6-((R)-1⁶-(2,6-dimethylphenyl)-3,3-dioxido-5-oxo-7-((1-(trifluoromethyl)cyclopropyl)methyl)-9-oxa-3-thia-2,6-diaza-1(2,4)-pyrimidina-4(1,3)-benzenacyclononaphane-6-yl)spiro[3.3]heptan-2-yl)carbamate (73 g, 96.58 mmol) in MeOH (400 mL) was added HCl (100 mL of 4 M, 400.0 mmol) portionwise. The mixture was stirred at ambient temperature for 20 hours. The solvent was removed in vacuo and the off-white solid slurried with MTBE and concentrated. The solid was dried under high vac for 48 h affording an off-white powder. (R)-6-((2S,4s,6S)-6-aminospiro[3.3]heptan-2-yl)-1⁶-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt) (60.0 g, 90%) ¹H NMR (400 MHz, DMSO-d₆) δ 8.33 (s, 1H), 8.19 (d, J=5.5 Hz, 3H), 7.89 (d, J=7.4 Hz, 1H), 7.72-7.59 (m, 2H), 7.26 (t, J=7.6 Hz, 1H), 7.12 (d, J=7.6 Hz, 2H), 6.39 (s, 1H), 5.04 (dd, J=10.8, 4.5 Hz, 1H), 4.32 (t, J=11.3 Hz, 1H), 4.05 (td, J=10.7, 4.3 Hz, 1H), 3.81 (p, J=8.7 Hz, 1H), 3.56 (h, J=7.9, 7.0 Hz, 1H), 3.04 (t, J=9.6 Hz, 1H), 2.95 (t, J=10.0 Hz, 1H), 2.45 (dt, J=11.7, 5.9 Hz, 1H), 2.29 (p, J=6.3, 5.4 Hz, 2H), 2.26-2.12 (m, 5H), 2.12-1.74 (m, 6H), 1.51 (dd, J=16.5, 9.4 Hz, 1H), 0.80 (dtd, J=19.9, 10.1, 5.0 Hz, 2H), 0.65 (dt, J=9.3, 4.8 Hz, 1H), 0.55 (dt, J=10.7, 5.0 Hz, 1H). ESI-MS m/z calc. 655.244, found 656.4 (M+1)⁺; Retention time: 1.73 minutes (LC method I).

### Step 6: Isopropyl ((2S,4s,6S)-6-((R)-1⁶-(2,6-dimethylphenyl)-3,3-dioxido-5-oxo-7-((1-(trifluoromethyl)cyclopropyl)methyl)-9-oxa-3-thia-2,6-diaza-1(2,4)-pyrimidina-4(1,3)-benzenacyclononaphane-6-yl)spiro[3.3]heptan-2-yl)carbamate (Compound 78)

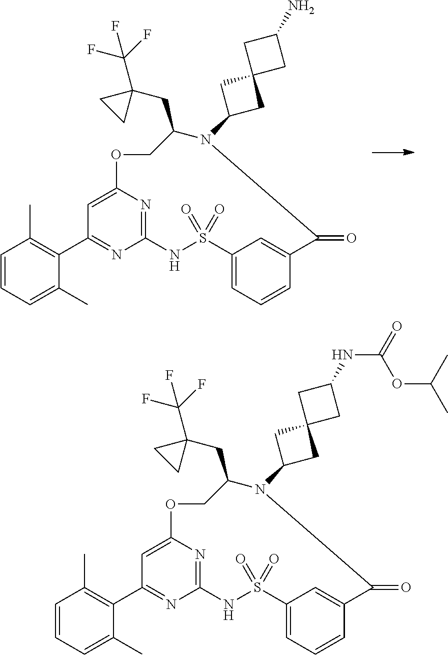

To a slurry of (R)-6-((2S,4s,6S)-6-aminospiro[3.3]heptan-2-yl)-1⁶-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt) (23.5 g, 33.95 mmol) in DCM (140 mL) was added DIEA (12 mL, 68.89 mmol)—gave an off white precipitate. Added 2MeTHF (140 mL) and observed some solubility. Added isopropyl chloroformate (42 mL of 1 M in toluene, 42.00 mmol) dropwise. The slurry was stirred at ambient temperature and was still a slurry after 16 h—Additional DIEA (6 mL, 34.45 mmol) and isopropyl chloroformate (12 mL of 1 M in toluene, 12.00 mmol) were added and the mixture stirred for an additional 1 h (17 h total). The mixture became homogenous, and the reaction was diluted with EtOAc (300 mL) and washed twice with HCl (250 mL of 1 M, 250.0 mmol) followed by 300 mL of brine. The organic phase was dried over magnesium sulfate, filtered and concentrated in vacuo. The off-white foam was dissolved in hot EtOAc (100 mL) and filtered through a pad of Celite. The solution was slowly added to Heptane (250 mL) with rapid stirring. The slurry was stirred at ambient temperature for 1 h. The solid was collected using a M frit and washing 3×with 50 mL of 1:2 EtOAc/heptane. The solid was air dried for 1 h, then in a vacuum oven at 45° C. for 48 h affording an off-white powder. isopropyl ((2S,4s,6S)-6-((R)-1⁶-(2,6-dimethylphenyl)-3,3-dioxido-5-oxo-7-((1-(trifluoromethyl)cyclopropyl)methyl)-9-oxa-3-thia-2,6-diaza-1(2,4)-pyrimidina-4(1,3)-benzenacyclononaphane-6-yl)spiro[3.3]heptan-2-yl)carbamate (21.8 g, 86%) ¹H NMR (400 MHz, DMSO-d₆) δ 13.03 (s, 1H), 8.32 (s, 1H), 7.87 (s, 1H), 7.65 (s, 2H), 7.26 (d, J=7.9 Hz, 2H), 7.12 (s, 2H), 6.37 (s, 1H), 5.02 (dd, J=10.9, 4.4 Hz, 1H), 4.72 (p, J=6.3 Hz, 1H), 4.33 (t, J=11.5 Hz, 1H), 4.04 (s, 1H), 3.81 (dq, J=40.0, 8.3 Hz, 2H), 3.02 (t, J=9.5 Hz, 1H), 2.92 (t, J=9.9 Hz, 1H), 2.40 (d, J=11.8 Hz, 1H), 2.31-2.03 (m, 6H), 1.95 (dd, J=18.6, 9.1 Hz, 6H), 1.49 (dd, J=16.5, 9.4 Hz, 1H), 1.15 (d, J=6.2 Hz, 6H), 0.91-0.71 (m, 2H), 0.62 (d, J=25.5 Hz, 2H). ESI-MS m/z calc. 741.28076, found 742.1 (M+1)⁺; Retention time: 2.77 minutes (LC method I).

### Example 62: Preparation of Compound 79

### Step 1: Isopropyl ((2R,4r,6R)-6-((R)-1⁶-(2,6-dimethylphenyl)-3,3-dioxido-5-oxo-7-((1-(trifluoromethyl)cyclopropyl)methyl)-9-oxa-3-thia-2,6-diaza-1(2,4)-pyrimidina-4(1,3)-benzenacyclononaphane-6-yl)spiro[3.3]heptan-2-yl)carbamate (Compound 79)

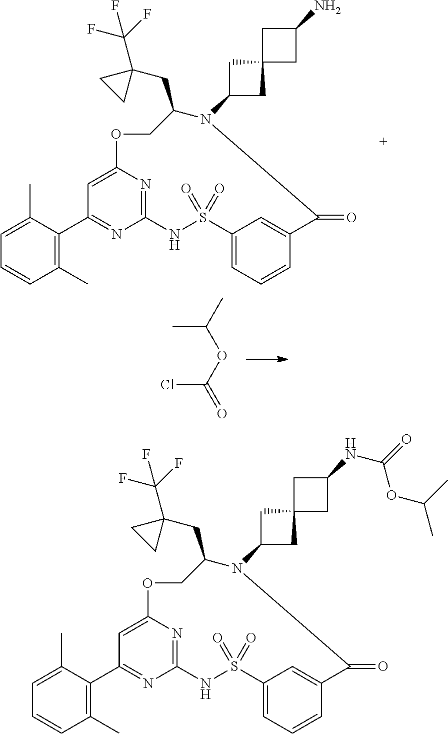

(R)-6-((2R,4r,6R)-6-Aminospiro[3.3]heptan-2-yl)-16-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt) (32 mg, 0.04623 mmol) was combined with the isopropyl chloroformate (approximately 46.23 μL of 2 M in toluene, 0.09246 mmol) in DCM (0.5 mL), and DIEA (approximately 29.88 mg, 40.27 μL, 0.2312 mmol) was added. The reaction was stirred at room temperature for one hour then was quenched with several drops of 1M HCl. The reaction mixture was partially concentrated, diluted with methanol and DMSO, filtered, and purified by reverse phase HPLC (1-99% ACN in water, HCl modifier, 15 min run) to give as a white solid isopropyl ((2R,4r,6R)-6-((R)-16-(2,6-dimethylphenyl)-3,3-dioxido-5-oxo-7-((1-(trifluoromethyl)cyclopropyl)methyl)-9-oxa-3-thia-2,6-diaza-1(2,4)-pyrimidina-4(1,3)-benzenacyclononaphane-6-yl)spiro[3.3]heptan-2-yl)carbamate (21.8 mg, 64%). ESI-MS m/z calc. 741.28076, found 742.7 (M+1)⁺; Retention time: 1.89 minutes; LC method A. ¹H NMR (400 MHz, DMSO-d₆) δ 13.03 (s, 1H), 8.33 (s, 1H), 7.87 (s, 1H), 7.64 (s, 2H), 7.26 (d, J=8.0 Hz, 2H), 7.12 (s, 2H), 6.37 (s, 1H), 5.04 (d, J=9.1 Hz, 1H), 4.72 (p, J=6.3 Hz, 1H), 4.34 (s, 1H), 4.05 (s, 1H), 3.91-3.73 (m, 2H), 2.95 (dt, J=25.5, 9.7 Hz, 2H), 2.32-2.03 (m, 7H), 2.01-1.84 (m, 6H), 1.48 (dd, J=16.7, 9.5 Hz, 1H), 1.15 (d, J=6.2 Hz, 6H), 0.77 (d, J=12.0 Hz, 2H), 0.60 (d, J=20.3 Hz, 2H).

### Example 63: Preparation of Compound 80

### Step 1: (R)-6-((2S,4s,6S)-6-Aminospiro[3.3]heptan-2-yl)-1⁶-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (Compound 80)

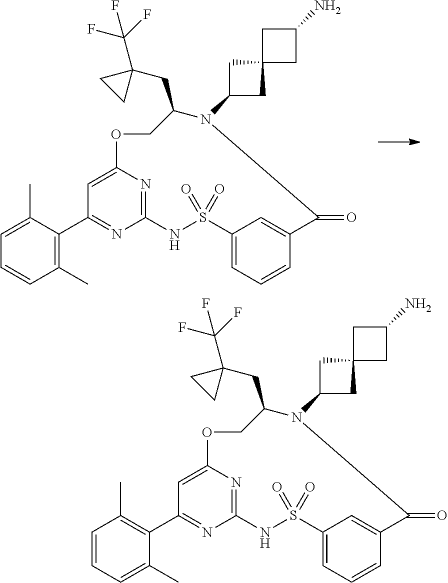

(R)-6-((2S,4s,6S)-6-Aminospiro[3.3]heptan-2-yl)-1⁶-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt) (30 mg, 0.04334 mmol) was dissolved in 1:1 methanol/DMSO, filtered, and purified by reverse phase HPLC (1-70% ACN in water, HCl modifier 15 min run) to give (R)-6-((2S,4s,6S)-6-aminospiro[3.3]heptan-2-yl)-16-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt) (23.1 mg, 76%). ESI-MS m/z calc. 655.244, found 656.6 (M+1)⁺; Retention time: 1.25 minutes; LC method A. ¹H NMR (400 MHz, DMSO-d₆) δ 13.07 (s, 1H), 8.33 (s, 1H), 7.98 (s, 2H), 7.88 (s, 1H), 7.65 (s, 2H), 7.25 (d, J=8.0 Hz, 1H), 7.12 (s, 2H), 6.37 (s, 1H), 5.04 (dd, J=10.9, 4.4 Hz, 1H), 4.31 (t, J=11.2 Hz, 1H), 4.04 (s, 1H), 3.86-3.75 (m, 1H), 3.57 (t, J=8.0 Hz, 1H), 3.29 (bs, 1H), 3.05 (t, J=9.6 Hz, 1H), 2.96 (t, J=10.0 Hz, 1H), 2.43 (d, J=10.8 Hz, 1H), 2.30 (s, 2H), 2.16 (d, J=16.8 Hz, 4H), 1.91 (s, 5H), 1.51 (dd, J=16.5, 9.4 Hz, 1H), 0.80 (dt, J=14.0, 5.6 Hz, 2H), 0.64 (s, 1H), 0.53 (s, 1H).

### Example 64: Preparation of Compound 81

### Step 1: (R)-6-((2R,4r,6R)-6-Aminospiro[3.3]heptan-2-yl)-1⁶-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (Compound 81)

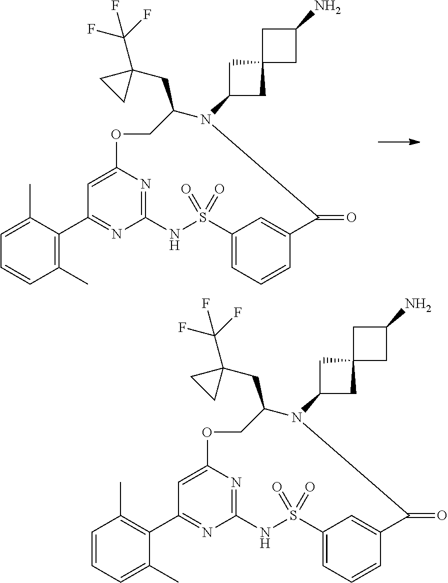

(R)-6-((2R,4r,6R)-6-Aminospiro[3.3]heptan-2-yl)-1⁶-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt) (30 mg, 0.04334 mmol) was dissolved in 1:1 methanol/DMSO, filtered, and purified by reverse phase HPLC (1-70% ACN in water, HCl modifier 15 min run) to give (R)-6-((2R,4r,6R)-6-aminospiro[3.3]heptan-2-yl)-16-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt) (26.4 mg, 87%). ESI-MS m/z calc. 655.244, found 656.6 (M+1)⁺; Retention time: 1.25 minutes; LC method A. ¹H NMR (400 MHz, DMSO-d₆) δ 13.05 (s, 1H), 8.34 (s, 1H), 8.02 (s, 2H), 7.88 (s, 1H), 7.65 (s, 2H), 7.25 (d, J=8.2 Hz, 1H), 7.12 (s, 2H), 6.38 (s, 1H), 5.05 (dd, J=10.9, 4.4 Hz, 1H), 4.32 (t, J=11.3 Hz, 1H), 4.05 (s, 1H), 3.81 (p, J=8.6 Hz, 1H), 3.58 (t, J=8.0 Hz, 1H), 3.29 (bs, 1H), 3.04 (t, J=9.8 Hz, 1H), 2.95 (t, J=9.8 Hz, 1H), 2.45 (t, J=6.0 Hz, 1H), 2.31 (d, J=9.1 Hz, 2H), 2.23-2.12 (m, 4H), 1.92 (s, 5H), 1.49 (dd, J=16.5, 9.4 Hz, 1H), 0.79 (q, J=10.8, 8.4 Hz, 2H), 0.58 (d, J=28.4 Hz, 2H).

### Example 65: Preparation of Compound 82

### Step 1: (R)-6-((2S,4s,6S)-6-(Benzylamino)spiro[3.3]heptan-2-yl)-16-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide

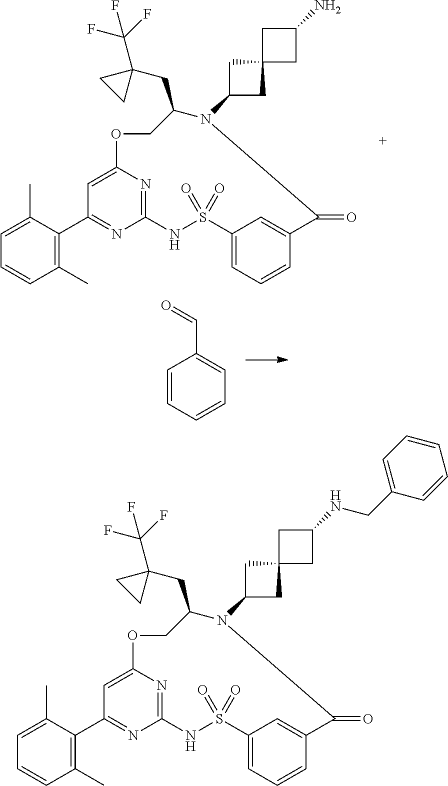

(R)-6-((2S,4s,6S)-6-Aminospiro[3.3]heptan-2-yl)-1⁶-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt) (500 mg, 0.7223 mmol) was combined with benzaldehyde (70 μL, 0.6886 mmol) in dichloromethane (2.4 mL), and stirred for 20 minutes at room temperature. Sodium triacetoxyborohydride (630 mg, 2.973 mmol) (added in two portions over 10 minutes) was then added and the reaction was allowed to stir for an additional 2 hours at room temperature. The reaction mixture was then partitioned between 1M HCl and ethyl acetate. The layers were separated, and the aqueous was extracted an additional 5×with ethyl acetate. The combined organics were washed with brine, dried over sodium sulfate, and concentrated to give as a white solid (with some double benzylation) (R)-6-((2S,4s,6S)-6-(benzylamino)spiro[3.3]heptan-2-yl)-16-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (436 mg, 81%) ESI-MS m/z calc. 745.29095, found 746.3 (M+1)⁺; Retention time: 0.6 minutes, LC method D.

### Step 2: (R)-1⁶-(2,6-Dimethylphenyl)-6-((2S,4s,6S)-6-(methylamino)spiro[3.3]heptan-2-yl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide

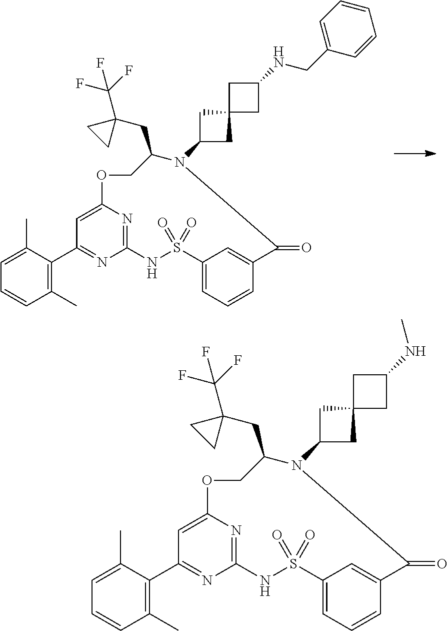

(R)-6-((2S,4s,6S)-6-(Benzylamino)spiro[3.3]heptan-2-yl)-16-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide was combined with aqueous formaldehyde (4 mL, 37% w/w, 145.2 mmol) and formic acid (3 mL, 79.52 mmol) in a screwcap vial and heated to 95° C. for 16 hours. The reaction mixture was then cooled to room temperature and partially concentrated under reduced pressure. Methanol and acetonitrile were added and the reaction mixture was concentrated a second time, then purified by reverse phase HPLC (1-99 acetonitrile in water, HCl modifier, and concentrated to give the N-methylated compound, (R)-6-((2S,4s,6S)-6-(benzyl(methyl)amino)spiro[3.3]heptan-2-yl)-16-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide as a white solid. The product was combined with wet dihydroxypalladium (70 mg, 10% w/w, 0.4985 mmol) in a nitrogen purged flask and methanol (10 mL) was added. hydrogen gas from a balloon was bubbled through the reaction mixture for 30 minutes, and the reaction was allowed to stir for an additional 2 hours at room temperature with the hydrogen balloon in place. After this time the reaction vessel was purged with nitrogen. The reaction mixture was then diluted with methanol and filtered through Celite (eluting with additional methanol) to give as a white solid upon drying, (R)-1⁶-(2,6-dimethylphenyl)-6-((2S,4s,6S)-6-(methylamino)spiro[3.3]heptan-2-yl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt) (195 mg, 58%). ESI-MS m/z calc. 669.25964, found 670.8 (M+1)⁺; Retention time: 0.52 minutes, LC method D.

### Step 3: (R)-1⁶-(2,6-Dimethylphenyl)-6-((2S,4s,6S)-6-((2-methoxyethyl)(methyl)amino)spiro[3.3]heptan-2-yl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (Compound 82)

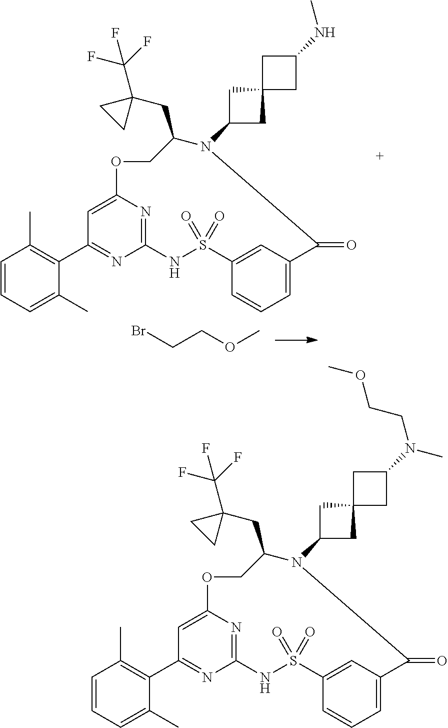

(R)-1⁶-(2,6-Dimethylphenyl)-6-((2S,4s,6S)-6-(methylamino)spiro[3.3]heptan-2-yl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt). (54 mg, 0.08063 mmol) was combined with 1-bromo-2-methoxy-ethane (approximately 33.62 mg, 22.73 μL, 0.2419 mmol) in acetonitrile, and triethylamine (approximately 40.80 mg, 56.20 μL, 0.4032 mmol) was added. The reaction mixture was then heated to 60° C. for 16 hours, and to 70° C. for an additional 6 hours. The reaction was cooled to room temperature, diluted with methanol, filtered and purified by reverse phase HPLC (1-70% ACN in water, HCl modifier, 15 min run) to give (R)-1⁶-(2,6-dimethylphenyl)-6-((2S,4s,6S)-6-((2-methoxyethyl)(methyl)amino)spiro[3.3]heptan-2-yl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt) (39.5 mg, 63%). ESI-MS m z calc. 727.3015, found 728.7 (M+1)⁺; Retention time: 1.33 minutes; LC method A. ¹H NMR (400 MHz, DMSO-d₆) δ 13.04 (s, 1H), 9.79 (s, 1H), 8.34 (s, 1H), 7.89 (s, 1H), 7.66 (s, 2H), 7.26 (t, J=7.7 Hz, 1H), 7.12 (d, J=7.6 Hz, 2H), 6.39 (s, 1H), 5.05 (dd, J=10.8, 4.5 Hz, 1H), 4.31 (t, J=11.3 Hz, 1H), 4.06 (d, J=11.4 Hz, 1H), 3.85 (p, J=8.6 Hz, 1H), 3.71-3.56 (m, 3H), 3.28-3.17 (m, 1H), 3.05 (t, J=9.6 Hz, 2H), 2.97 (t, J=10.0 Hz, 1H), 2.71-2.60 (m, 3H), 2.53 (d, J=6.8 Hz, 1H), 2.48 (s, 8H), 2.12-1.72 (m, 5H), 1.59-1.45 (m, 1H), 1.27-1.22 (m, 1H), 0.85-0.72 (m, 2H), 0.67-0.58 (m, 1H), 0.52 (s, 1H).

### Example 66: Preparation of Compound 83

### Step 1: (R)-6-((2R,4r,6R)-6-(Benzylamino)spiro[3.3]heptan-2-yl)-16-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide

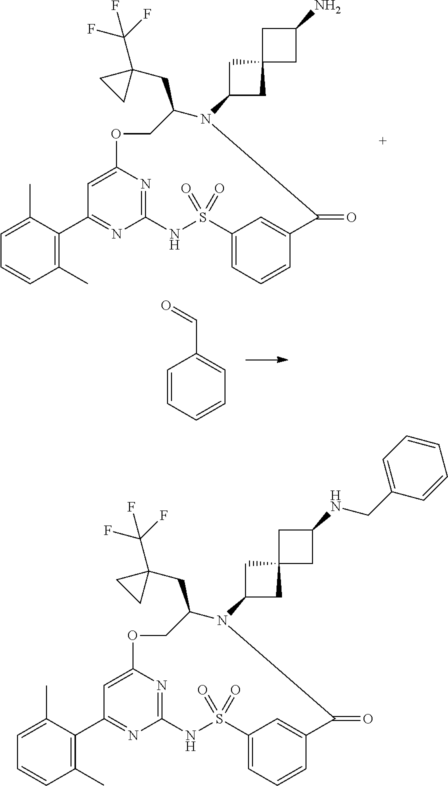

((R)-6-(2R,4r,6R-6-Aminospiro[33]heptan-2-yl)-1⁶-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt) (500 mg, 0.7223 mmol) was combined with benzaldehyde (70 μL, 0.6886 mmol) in dichloromethane (2.4 mL), and stirred for 30 minutes at room temperature. Sodium triacetoxyborohydride (630 mg, 2.973 mmol) was then added and the reaction was allowed to stir for two hours at room temperature. The reaction mixture was then partitioned between 1M HCl and ethyl acetate. The layers were separated, and the aqueous was extracted an additional 4×with ethyl acetate. The combined organics were washed with brine, dried over sodium sulfate, and concentrated to give a white solid, (with a small amount of the double benzylation present) (R)-6-((2R,4r,6R)-6-(benzylamino)spiro[3.3]heptan-2-yl)-16-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (466 mg, 87%) ESI-MS m/z calc. 745.29095, found 746.5 (M+1)⁺; Retention time: 0.56 minutes, LC method D.

### Step 2: (R)-1⁶-(2,6-Dimethylphenyl)-6-((2R,4r,6R)-6-(methylamino)spiro[3.3]heptan-2-yl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt)

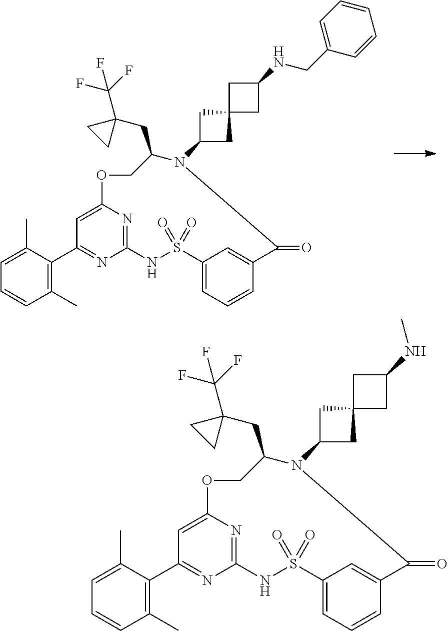

(R)-6-((2R,4r,6R)-6-(Benzylamino)spiro[3.3]heptan-2-yl)-16-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide was combined with aqueous formaldehyde (4 mL, 37% w/w, 145.2 mmol) and formic acid (3 mL, 79.52 mmol) in a screwcap vial and heated to 95° C. for 16 hours. The reaction mixture was then cooled to room temperature and partially concentrated under reduced pressure. Methanol and acetonitrile were added and the reaction mixture was concentrated a second time, then purified by reverse phase HPLC (1-99 acetonitrile in water, HCl modifier-eluted around 55% methanol), and concentrated to give the N-methylated compound, (R)-6-((2R,4r,6R)-6-(benzyl(methyl)amino)spiro[3.3]heptan-2-yl)-16-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide as a white solid. The product was combined with wet dihydroxypalladium (70 mg, 10% w/w, 0.4985 mmol) in a nitrogen purged flask and methanol (10 mL) was added. Hydrogen gas from a balloon was bubbled through the reaction mixture for 30 minutes, and the reaction was allowed to stir for an additional 2 hours at room temperature with the hydrogen balloon in place. After this time the reaction vessel was purged with nitrogen. The reaction mixture was then diluted with methanol and filtered through Celite (eluting with additional methanol) to give as a white solid upon drying, (R)-1⁶-(2,6-dimethylphenyl)-6-((2R,4r,6R)-6-(methylamino)spiro[3.3]heptan-2-yl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt) (230 mg, 66%). ESI-MS m/z calc. 669.25964, found 670.8 (M+1)⁺; Retention time: 0.52 minutes, LC method D.

### Step 3: (R)-16-(2,6-Dimethylphenyl)-6-((2R,4r,6R)-6-((2-methoxyethyl)(methyl)amino)spiro[3.3]heptan-2-yl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (Compound 83)

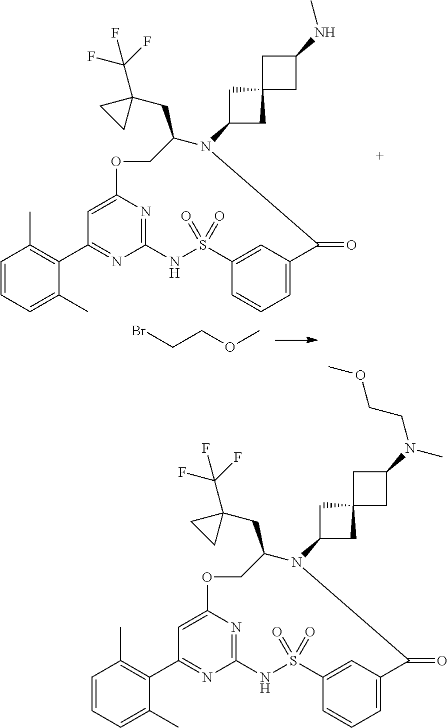

(R)-1⁶-(2,6-Dimethylphenyl)-6-((2R,4r,6R)-6-(methylamino)spiro[3.3]heptan-2-yl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt) (50 mg, 0.07465 mmol) was combined with 1-bromo-2-methoxy-ethane (approximately 31.12 mg, 21.04 μL, 0.2239 mmol) in acetonitrile, and triethylamine (approximately 37.76 mg, 52.01 μL, 0.3732 mmol) was added. The reaction mixture was then heated to 60° C. for 16 hours. After the indicated time, the reactions were cooled to room temperature, diluted with methanol, filtered and purified by reverse phase HPLC (1-70% ACN in water, HCl modifier, 15 min run) to give (R)-1⁶-(2,6-dimethylphenyl)-6-((2R,4r,6R)-6-((2-methoxyethyl)(methyl)amino)spiro[3.3]heptan-2-yl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt) (40.6 mg, 70%). ESI-MS m z calc. 727.3015, found 728.8 (M+1)⁺; Retention time: 1.32 minutes; LC method A. ¹H NMR (400 MHz, DMSO-d₆) δ 13.04 (s, 1H), 9.93 (s, 1H), 8.34 (s, 1H), 7.89 (d, J=7.2 Hz, 1H), 7.66 (s, 2H), 7.26 (t, J=7.6 Hz, 1H), 7.12 (d, J=7.6 Hz, 2H), 6.39 (s, 1H), 5.05 (dd, J=10.8, 4.5 Hz, 1H), 4.33 (t, J=11.3 Hz, 1H), 4.05 (t, J=9.6 Hz, 1H), 3.86 (p, J=8.6 Hz, 1H), 3.72-3.57 (m, 3H), 3.32 (s, 3H), 3.26-3.14 (m, 1H), 3.07 (dd, J=12.1, 7.8 Hz, 2H), 2.97 (t, J=9.7 Hz, 1H), 2.64 (d, J=4.7 Hz, 3H), 2.39-2.28 (m, 3H), 2.20-2.10 (m, 3H), 2.11-1.79 (m, 5H), 1.49 (dd, J=16.5, 9.3 Hz, 1H), 1.31-1.19 (m, 1H), 0.85-0.71 (m, 2H), 0.67-0.46 (m, 2H).

### Example 67: Preparation of Compound 84

### Step 1: N-((2S,4s,6S)-6-((R)-16-(2,6-Dimethylphenyl)-3,3-dioxido-5-oxo-7-((1-(trifluoromethyl)cyclopropyl)methyl)-9-oxa-3-thia-2,6-diaza-1(2,4)-pyrimidina-4(1,3)-benzenacyclononaphane-6-yl)spiro[3.3]heptan-2-yl)acetamide (Compound 84)

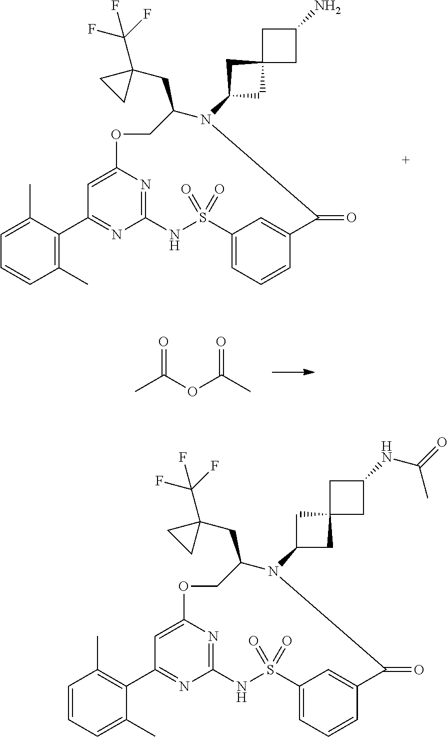

(R)-6-((2S,4s,6S)-6-aminospiro[3.3]heptan-2-yl)-1⁶-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt) (35 mg, 0.05056 mmol) was combined with acetic anhydride (approximately 10.32 mg, 9.538 μL, 0.1011 mmol) in DCM (0.5 mL) and triethylamine (approximately 25.58 mg, 35.23 μL, 0.2528 mmol) was added. The reaction was stirred at room temperature for 2 hours, then was partially concentrated, diluted with 1:1 methanol/DMSO, filtered, and purified by reverse phase HPLC (1-99% ACN in water, HCl modifier) to give N-((2S,4s,6S)-6-((R)-16-(2,6-dimethylphenyl)-3,3-dioxido-5-oxo-7-((1-(trifluoromethyl)cyclopropyl)methyl)-9-oxa-3-thia-2,6-diaza-1(2,4)-pyrimidina-4(1,3)-benzenacyclononaphane-6-yl)spiro[3.3]heptan-2-yl)acetamide (27.0 mg, 77%). ESI-MS m z calc. 697.2546, found 698.5 (M+1)⁺; Retention time: 1.55 minutes; LC method A. ¹H NMR (400 MHz, DMSO-d₆) δ 13.02 (s, 1H), 8.31 (s, 1H), 8.03 (d, J=7.7 Hz, 1H), 7.87 (s, 1H), 7.65 (s, 2H), 7.25 (s, 1H), 7.12 (s, 2H), 6.37 (s, 1H), 5.02 (d, J=8.8 Hz, 1H), 4.33 (s, 1H), 4.07 (q, J=8.0 Hz, 2H), 3.78 (t, J=8.6 Hz, 1H), 3.29 (bs, 2H), 3.04 (t, J=9.4 Hz, 1H), 2.95 (t, J=9.9 Hz, 1H), 2.40 (m, 1H), 2.25 (dt, J=12.0, 7.0 Hz, 2H), 2.20-1.98 (m, 4H), 2.00-1.84 (m, 4H), 1.75 (s, 3H), 1.49 (dd, J=16.5, 9.4 Hz, 1H), 0.78 (d, J=12.6 Hz, 2H), 0.62 (d, J=29.2 Hz, 2H).

### Example 68: Preparation of Compound 85

### Step 1: N-((2R,4r,6R)-6-((R)-1⁶-(2,6-Dimethylphenyl)-3,3-dioxido-5-oxo-7-((1-(trifluoromethyl)cyclopropyl)methyl)-9-oxa-3-thia-2,6-diaza-1(2,4)-pyrimidina-4(1,3)-benzenacyclononaphane-6-yl)spiro[3.3]heptan-2-yl)acetamide (Compound

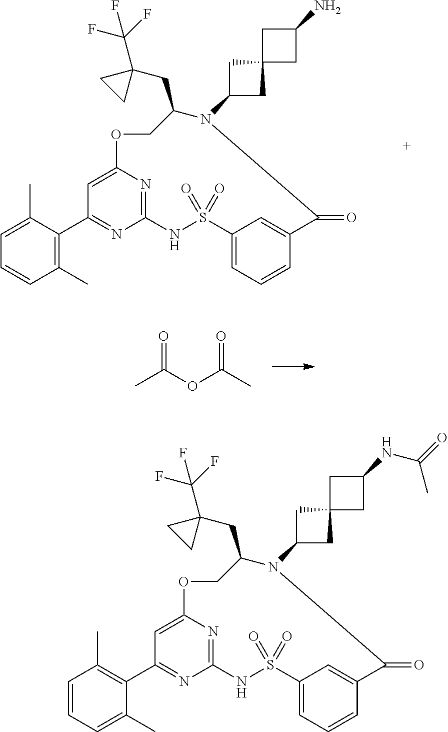

(R)-6-((2R,4r,6R)-6-aminospiro[3.3]heptan-2-yl)-1⁶-(2,6-dimethylphenyl)-7-((1-(trifluoromethyl)cyclopropyl)methyl)-9-oxa-3-thia-2,6-diaza-1(2,4)-pyrimidina-4(1,3)-benzenacyclononaphan-5-one 3,3-dioxide (hydrochloride salt) (35 mg, 0.05056 mmol) was combined with acetic anhydride (approximately 10.32 mg, 9.538 μL, 0.1011 mmol) in DCM (0.5 mL) and triethylamine (approximately 25.58 mg, 35.23 μL, 0.2528 mmol) was added. The reaction was stirred at room temperature for 2 hours, then was partially concentrated, diluted with 1:1 methanol/DMSO, filtered, and purified by reverse phase HPLC (1-99% ACN in water, HCl modifier) to give N-((2R,4r,6R)-6-((R)-16-(2,6-dimethylphenyl)-3,3-dioxido-5-oxo-7-((1-(trifluoromethyl)cyclopropyl)methyl)-9-oxa-3-thia-2,6-diaza-1(2,4)-pyrimidina-4(1,3)-benzenacyclononaphane-6-yl)spiro[3.3]heptan-2-yl)acetamide (25.5 mg, 72%). ESI-MS m z calc. 697.2546, found 698.6 (M+1)⁺; Retention time: 1.56 minutes; LC method A. ¹H NMR (400 MHz, DMSO-d₆) δ 13.03 (s, 1H), 8.33 (s, 1H), 8.02 (d, J=7.6 Hz, 1H), 7.87 (s, 1H), 7.64 (s, 2H), 7.25 (s, 1H), 7.12 (s, 2H), 6.37 (s, 1H), 5.04 (d, J=8.0 Hz, 1H), 4.34 (s, 1H), 4.07 (q, J=7.9 Hz, 2H), 3.78 (t, J=8.5 Hz, 1H), 3.29 (s, 1H), 2.97 (dd, J=29.8, 9.8 Hz, 2H), 2.46-2.38 (m, 1H), 2.32-2.20 (m, 2H), 2.20-1.79 (m, 9H), 1.75 (s, 3H), 1.48 (dd, J=16.3, 9.4 Hz, 1H), 0.78 (d, J=13.3 Hz, 2H), 0.60 (d, J=21.3 Hz, 2H).

### Example 69: Preparation of Compound 86 and Compound 87

### Step 1: Methyl 7,10-dioxadispiro[3.1.46.14]undecane-2-carboxylate

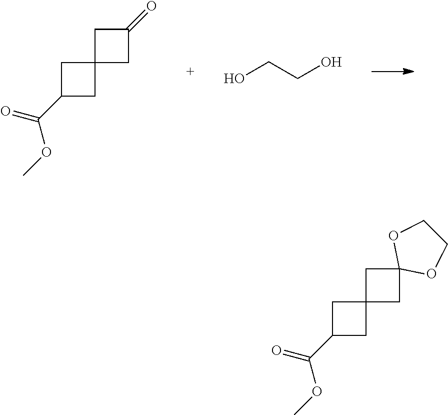

To a stirring solution of methyl 2-oxospiro[3.3]heptane-6-carboxylate (19.663 g, 116.91 mmol) and ethylene glycol (15.582 g, 14 mL, 251.05 mmol) in toluene (190 mL) at room temperature under ambient conditions was added p-toluenesulfonic acid hydrate (1.141 g, 5.9984 mmol). The reaction mixture was heated to reflux (140° C.) with Dean-Stark apparatus for 24 hours. After cooling to room temperature, the reaction mixture was quenched with saturated aqueous sodium bicarbonate (350 mL). Two layers were separated, and the aqueous layer was extracted with ethyl acetate (2×300 mL). The combined organic layers were washed with brine (150 mL), dried over anhydrous sodium sulfate and concentrated to afford methyl 7,10-dioxadispiro[3.1.46.14]undecane-2-carboxylate (27.67 g, 100%) as pale-yellow oil. The product was carried to the next step without further purification. ¹H NMR (250 MHz, CDCl₃) δ 4.34-4.13 (m, 2H), 3.91-3.79 (m, 5H), 3.15-2.93 (m, 1H), 2.49-2.37 (m, 4H), 2.35-2.26 (m, 4H).

### Step 2: 7,10-Dioxadispiro[3.1.46.14]undecan-2-yl(diphenyl)methanol

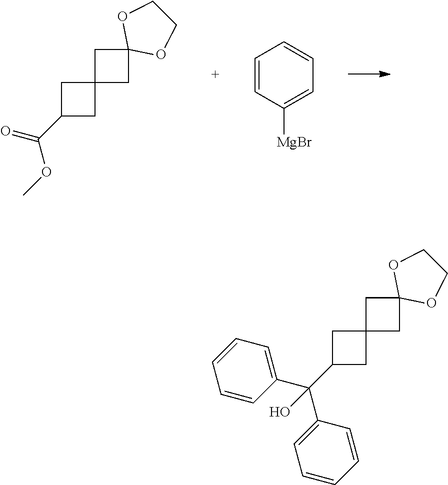

To a stirring solution of methyl 7,10-dioxadispiro[3.1.46.14]undecane-2-carboxylate (27.67 g, 117.33 mmol) in anhydrous diethyl ether (250 mL) at 0° C. under nitrogen was dropwise added a solution of bromo(phenyl)magnesium (135 mL of 3 M, 405.00 mmol) in diethyl ether. During this addition, a copious amount of precipitate was formed. After the addition was complete, the reaction mixture was stirred at this temperature for 10 minutes. The ice-water bath was removed, and the reaction mixture was heated to reflux (42° C.) for 2 hours. The reaction mixture was cooled to 0° C., and slowly quenched with saturated aqueous ammonium chloride (500 mL). The reaction mixture was allowed to warm up to room temperature and stirred until all the solid has dissolved. Two layers were separated, and the aqueous layer was extracted with diethyl ether (2×300 mL). The combined organic layers were washed with brine (150 mL), dried over anhydrous sodium sulfate and concentrated. The crude was purified by silica gel chromatography using 0-40% diethyl ether gradient in hexanes to afford 7,10-dioxadispiro[3.1.46.14]undecan-2-yl(diphenyl)methanol (28.07 g, 64%) as white solid. ESI-MS m/z calc. 336.1725, found 319.3 (M-water+H)⁺; Retention time: 5.77 minutes. H NMR (250 MHz, CDCl₃) δ 7.47-7.10 (m, 10H), 3.85 (s, 4H), 3.23 (p, J=8.7, 8.7, 8.6, 8.6 Hz, 1H), 2.40 (s, 2H), 2.25-2.10 (m, 5H), 2.04-1.89 (m, 2H). LC method S.

### Step 3: 2-Benzhydrylidene-7,10-dioxadispiro[3.1.46.14]undecane

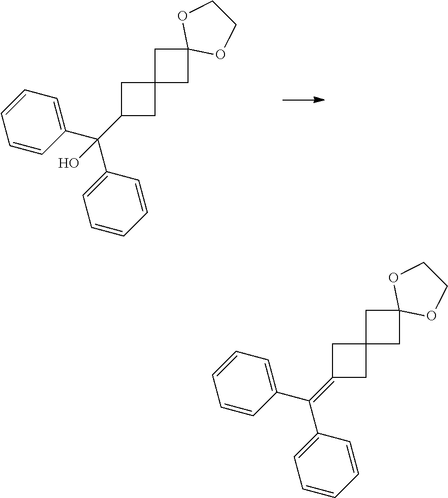 [SMILES: OC(c1ccccc1)(c1ccccc1)C1CC2(C1)CC1(C2)OCCO1 ; c1ccc(C(=C2CC3(C2)CC2(C3)OCCO2)c2ccccc2)cc1]

To a stirring solution of 7,10-dioxadispiro[3.1.46.14]undecan-2-yl(diphenyl)methanol (28.07 g, 83.436 mmol) in toluene (400 mL) at room temperature under ambient conditions was added p-toluenesulfonic acid hydrate (1.664 g, 8.7479 mmol). The reaction mixture was heated to reflux (140° C.) with Dean-Stark apparatus for 24 hours. After cooling to room temperature, volatiles were removed under vacuum. The obtained residue was dissolved in ethyl acetate (350 mL) and washed with saturated aqueous sodium bicarbonate (400 mL). Two layers were separated, and the aqueous layer was extracted with ethyl acetate (2×200 mL). The combined organic layers were washed with brine (150 mL), dried over anhydrous sodium sulfate and concentrated to afford 2-benzhydrylidene-7,10-dioxadispiro[3.1.46.14]undecane (26.645 g, 90%) as yellow solid. The product was carried to the next step without further purification. ESI-MS m/z calc. 318.162, found 319.0 (M+1)⁺; Retention time: 7.17 minutes ¹H NMR (250 MHz, CDCl₃) δ 7.43-7.04 (m, 10H), 3.88 (s, 4H), 3.03 (s, 4H), 2.43 (s, 4H). LC method S.

### Step 4: 7,10-Dioxadispiro[3.1.46.14]undecan-2-one

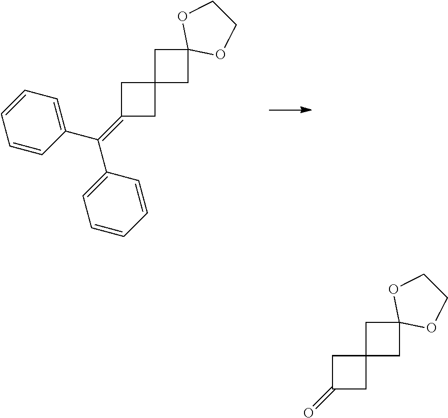 [SMILES: c1ccc(C(=C2CC3(C2)CC2(C3)OCCO2)c2ccccc2)cc1 ; O=C1CC2(C1)CC1(C2)OCCO1]

To a stirring solution of 2-benzhydrylidene-7,10-dioxadispiro[3.1.46.14]undecane (26.645 g, 83.682 mmol) in a mixture of acetonitrile (350 mL) and carbon tetrachloride (350 mL) at room temperature under ambient conditions was added water (550 mL). To the reaction mixture was added ruthenium(III) chloride hydrate (1.902 g, 8.4367 mmol), followed by a portionwise addition of sodium periodate (90.18 g, 421.61 mmol). After the addition was complete, the reaction mixture was stirred at this temperature for 5 minutes. The reaction mixture was heated to reflux (82° C.) for 1 hour. The reaction mixture was allowed to cool down to room temperature and filtered through a pad of Celite. The filter cake was washed with chloroform (3×200 mL). The combined filtrate was concentrated under vacuum to remove the volatiles. The residual aqueous layer was diluted with brine (200 mL), and the product was extracted with chloroform (3×400 mL). The combined organic layers were washed with brine (200 mL), dried over anhydrous sodium sulfate and concentrated. The crude was purified by silica gel chromatography using 0-20% acetone gradient in hexanes to afford 7,10-dioxadispiro[3.1.46.14]undecan-2-one (8.745 g, 59%) as a yellow oil. ¹H NMR (250 MHz, CDCl₃) δ 3.91 (s, 4H), 3.18 (s, 4H), 2.59 (s, 4H).

### Step 5: 3-[[4-(2,6-Dimethylphenyl)-6-[(2R)-2-(7,10-dioxadispiro[3.1.46.14]undecan-2-ylamino)-4,4-dimethyl-pentoxy]pyrimidin-2-yl]sulfamoyl]benzoic acid

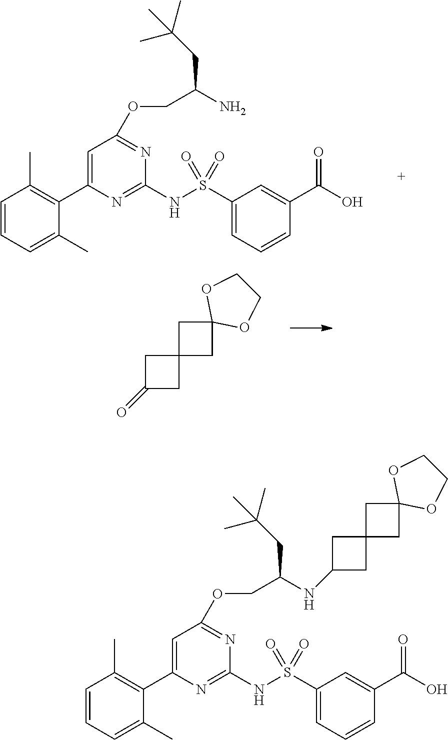

To a stirring suspension of 3-[[4-[(2R)-2-amino-4,4-dimethyl-pentoxy]-6-(2,6-dimethylphenyl)pyrimidin-2-yl]sulfamoyl]benzoic acid (hydrochloride salt) (8.656 g, 15.765 mmol) and 7,10-dioxadispiro[3.1.46.14]undecan-2-one (3.195 g, 18.996 mmol) in 1,2-dichloroethane (120 mL) at room temperature under ambient conditions was portionwise added sodium triacetoxyborohydride (10.26 g, 48.410 mmol). After the addition was complete, within 15 minutes the reaction mixture became a homogeneous solution. The reaction mixture was stirred at this temperature for 18 hours. The reaction mixture was cooled to 0° C., and slowly quenched with saturated aqueous ammonium chloride (400 mL). Chloroform (150 mL) was added to the cold mixture, and the reaction mixture was allowed to warm up to room temperature. Two layers were separated, and the aqueous layer was extracted with chloroform (2×200 mL). The combined organic layers were washed with brine (100 mL) and dried over anhydrous sodium sulfate. The crude was concentrated under vacuum to a residual volume of ˜60 mL, loaded directly onto a silica gel column and purified using 0-10% methanol gradient in dichloromethane to afford 3-[[4-(2,6-dimethylphenyl)-6-[(2R)-2-(7,10-dioxadispiro[3.1.46.14]undecan-2-ylamino)-4,4-dimethyl-pentoxy]pyrimidin-2-yl]sulfamoyl]benzoic acid (6.67 g, 59%) as white solid. ESI-MS m/z calc. 664.2931, found 665.5 (M+1)⁺; Retention time: 4.2 minutes. LC method S.

### Step 6: (11R)-6-(2,6-Dimethylphenyl)-11-(2,2-dimethylpropyl)-12-(7,10-dioxadispiro[3.1.46.14]undecan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one

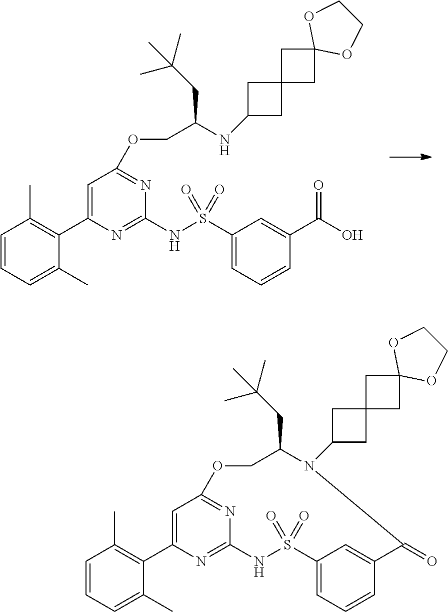 [SMILES: Cc1cccc(C)c1-c1cc(OC[C@@H](CC(C)(C)C)NC2CC3(C2)CC2(C3)OCCO2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(C1)CC1(C3)OCCO1)[C@H](CC(C)(C)C)CO2]

Into a solution of 3-[[4-(2,6-dimethylphenyl)-6-[(2R)-2-(7,10-dioxadispiro[3.1.46.14]undecan-2-ylamino)-4,4-dimethyl-pentoxy]pyrimidin-2-yl]sulfamoyl]benzoic acid (8.57 g, 12.246 mmol) in anhydrous DMF (171 mL) was added COMU (8 g, 18.306 mmol) at 0° C. DIEA (4.7488 g, 6.4 mL, 36.743 mmol) was added to the reaction mixture dropwise. The reaction was stirred at rt overnight. The reaction was quenched with a mixture of 10% citric acid (100 mL) and water (100 mL), and then it was extracted with ethyl acetate (3×200 mL). The combined organic layers were washed with brine (3×100 mL), dried over anhydrous sodium sulfate and concentrated under vacuum. The residue was purified by silica gel chromatography using 10 to 40% acetone in hexane to furnish (11R)-6-(2,6-dimethylphenyl)-11-(2,2-dimethylpropyl)-12-(7,10-dioxadispiro[3.1.46.14]undecan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one (6.7 g, 76%) as a red foamy solid. ESI-MS m/z calc. 646.2825, found 647.4 (M+1)⁺; Retention time: 3.32 minutes. LC method T.

### Step 7: (11R)-6-(2,6-Dimethylphenyl)-11-(2,2-dimethylpropyl)-2,2-dioxo-12-(2-oxospiro[3.3]heptan-6-yl)-9-oxa-2λ⁶-thia-3,5,12,19-tetrazatricyclo[12.3.1.14,8]nonadeca-1(18),4(19),5,7,14,16-hexaen-13-one

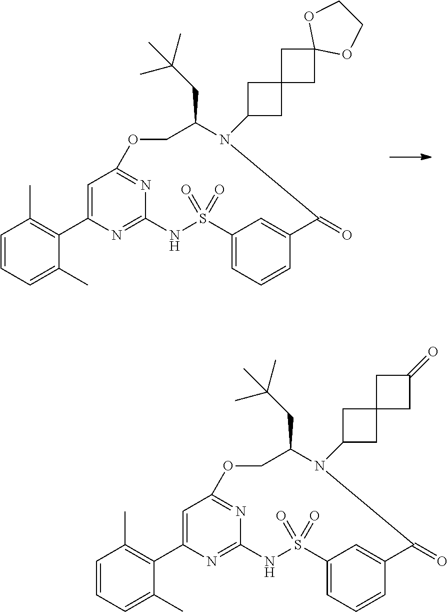 [SMILES: Cc1cccc(C)c1-c1cc2nc(n1)NS(=O)(=O)c1cccc(c1)C(=O)N(C1CC3(C1)CC1(C3)OCCO1)[C@H](CC(C)(C)C)CO2 ; Cc1cccc(C)c1-c1cc2nc(n1)NS(=O)(=O)c1cccc(c1)C(=O)N(C1CC3(CC(=O)C3)C1)[C@H](CC(C)(C)C)CO2]

Into a solution of (11R)-6-(2,6-dimethylphenyl)-11-(2,2-dimethylpropyl)-12-(7,10-dioxadispiro[3.1.46.14]undecan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one (6.7 g, 9.3229 mmol) in acetone (100 mL) was added pTSA hydrate (176 mg, 0.9253 mmol). The reaction was stirred at 60° C. in an oil bath for 20 hours. Another portion of pTSA hydrate (176 mg, 0.1645 mL, 0.9253 mmol) was added. The reaction was stirred for another 3 hours at 60° C. The reaction was cooled to rt, and then it was concentrated under vacuum. The residue was purified by silica gel chromatography using 10 to 40% acetone in hexane to furnish (11R)-6-(2,6-dimethylphenyl)-11-(2,2-dimethylpropyl)-2,2-dioxo-12-(2-oxospiro[3.3]heptan-6-yl)-9-oxa-2λ⁶-thia-3,5,12,19-tetrazatricyclo[12.3.1.14,8]nonadeca-1(18),4(19),5,7,14,16-hexaen-13-one (4.5593 g, 77%) as a light yellow solid. ESI-MS m/z calc. 602.2563, found 603.8 (M+1)⁺; Retention time: 2.65 minutes. ¹H NMR (500 MHz, DMSO-d₆) δ 13.05 (s, 1H), 8.45 (s, 1H), 7.94 (d, J=12.1 Hz, 1H), 7.68 (s, 2H), 7.25 (d, J=7.9 Hz, 1H), 7.12 (s, 2H), 6.41 (s, 1H), 5.10 (dd, J=10.7, 4.4 Hz, 1H), 4.32 (t, J=10.5, 10.5 Hz, 1H), 4.13-3.95 (m, 1H), 3.79-3.63 (m, 1H), 3.32-3.29 (m, 1H), 3.25 (t, J=9.8, 9.8 Hz, 1H), 3.21 (s, 2H), 3.15 (s, 2H), 2.48-2.41 (m, 2H), 2.25-1.77 (m, 6H), 1.62 (dd, J=15.2, 8.4 Hz, 1H), 1.39 (d, J=14.9 Hz, 1H), 0.50 (s, 9H). LC method W.

### Step 8: (11R)-12-[6-[(2S,6R)-2,6-dimethylmorpholin-4-yl]spiro[3.3]heptan-2-yl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4,6,8(19),14,16-hexaen-13-one, diastereomer 1 (Compound 87), and (11R)-12-[6-[(2S,6R)-2,6-dimethylmorpholin-4-yl]spiro[3.3]heptan-2-yl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4,6,8(19),14,16-hexaen-13-one, diastereomer 2 (Compound 86)

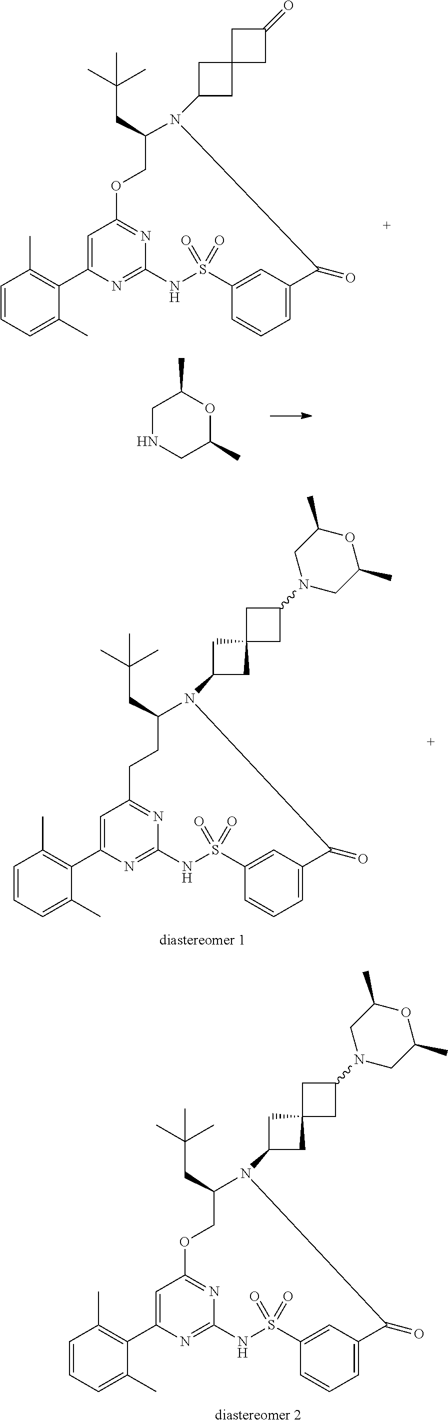

(11R)-6-(2,6-Dimethylphenyl)-11-(2,2-dimethylpropyl)-12-{6-oxospiro[3.3]heptan-2-yl}-9-oxa-2λ⁶-thia-3,5,12,19-tetraazatricyclo[12.3.1.14,8]nonadeca-1(17),4(19),5,7,14(18),15-hexaene-2,2,13-trione (50 mg, 0.08295 mmol) and (2S,6R)-2,6-dimethylmorpholine (20 mg, 0.1737 mmol) were combined and dissolved in dichloromethane (0.50 mL). The mixture was stirred at room temperature for 15 minutes. sodium triacetoxyborohydride (53 mg, 0.2501 mmol) was added, and the reaction mixture was stirred at room temperature for 30 minutes. The reaction mixture was filtered and the product was isolated by UV-triggered reverse phase HPLC eluting with a 10-99% acetonitrile/water gradient over 15 minutes with 0.5 mM HCl acid modifier in the aqueous phase to give two isomers: diastereomer 1, peak 1: (11R)-12-[6-[(2S,6R)-2,6-dimethylmorpholin-4-yl]spiro[3.3]heptan-2-yl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4,6,8(19),14,16-hexaen-13-one (15.9 mg, 55%), ESI-MS m/z calc. 701.3611, found 702.7 (M+1)⁺; Retention time: 1.41 minutes, LC method A; and diastereomer 2, peak 2: (11R)-12-[6-[(2S,6R)-2,6-dimethylmorpholin-4-yl]spiro[3.3]heptan-2-yl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4,6,8(19),14,16-hexaen-13-one (9.3 mg, 32%). ESI-MS m/z calc. 701.3611, found 702.7 (M+1)⁺; Retention time: 1.44 minutes, LC method A.

### Example 70: Preparation of Compound 88, Compound 89, and Compound 90

### Step 1: tert-Butyl N-[6-[[(1R)-1-(hydroxymethyl)-3,3-dimethyl-butyl]amino]spiro[3.3]heptan-2-yl]carbamate

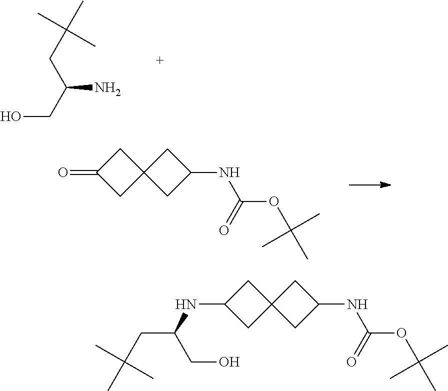

To a mixture of (2R)-2-amino-4,4-dimethyl-pentan-1-ol (hydrochloride salt) (55 g, 328.0 mmol) in 1,2-dichloroethane (600 mL) was added DIEA (60 mL, 344.5 mmol) and the mixture stirred for 5 min at ambient temperature. To the mixture was added tert-butyl N-(2-oxospiro[3.3]heptan-6-yl)carbamate (73.7 g, 327.1 mmol) followed by HOAc (20 mL, 351.7 mmol) and the homogenous mixture stirred for 2 h. To the mixture was added sodium triacetoxyborohydride (83.6 g, 394.4 mmol) portionwise and the mixture stirred at ambient temperature for 2 h. The mixture was cooled with an ice-water bath and quenched with water (600 mL) and stirred for 10 min. To the mixture was added HCl (60 mL of 12 M, 720.0 mmol) portionwise until the mixture had a ˜pH 1, followed by isopropyl Acetate (600 mL). The mixture was basified with NaOH (160 g of 50% w/w, 2.000 mol) resulting in an emulsion. After adding NaCl, adjusting pH lower, and adding iPrOAc the organic phase was partially separated, and the solvent was removed in vacuo to about 250 mL. The aqueous phase was passed through a plug of Celite. The aqueous phase was extracted with 1 L of iPrOAc. The organic phases were combined and filtered through a plug of Celite. A small amount of water separated, and the organic phase was dried over magnesium sulfate, filtered over Celite and concentrated in vacuo affording light yellow molasses. It was diluted with MTBE (1,000 mL) and TsOH (42 g, 243.9 mmol) was added. The mixture was stirred at ambient temperature for 4 h. The off-white slurry was filtered using an M frit to give a solid paste. The precipitate was air dried for 20 h. The still damp solid was then diluted with MTBE (1000 mL) and the precipitate residue transferred with MeOH (100 mL). To the milky solution was added NaOH (350 mL of 2 M, 700.0 mmol) and the mixture stirred until no solid was observed. The organic phase was separated, and the aqueous phase extracted with MTBE (1000 mL). The combined organic phases were washed with 300 mL of brine, dried over magnesium sulfate, filtered and concentrated in vacuo affording a light yellow oily foam of tert-butyl N-[6-[[(1R)-1-(hydroxymethyl)-3,3-dimethyl-butyl]amino]spiro[3.3]heptan-2-yl]carbamate (88.5 g, 79%). ¹H NMR (400 MHz, DMSO-d₆) δ 7.00 (d, J=8.0 Hz, 1H), 4.42 (s, 1H), 4.09 (q, J=5.4 Hz, OH), 3.78 (q, J=8.1 Hz, 1H), 3.26 (dd, J=10.7, 4.5 Hz, 1H), 3.21-3.10 (m, 2H), 2.42 (q, J=5.5, 5.0 Hz, 1H), 2.23 (dp, J=18.3, 6.7, 6.2 Hz, 2H), 2.05 (dt, J=11.6, 5.4 Hz, 2H), 1.82 (q, J=9.8, 9.3 Hz, 2H), 1.71-1.42 (m, 3H), 1.35 (s, 10H), 0.87 (s, 10H). ESI-MS m/z calc. 340.27258, found 341.3 (M+1)⁺; Retention time: 0.9 minutes (LC method A).

### Step 2: 3-[[4-[(2R)-2-[[2-(tert-Butoxycarbonylamino)spiro[3.3]heptan-6-yl]amino]-4,4-dimethyl-pentoxy]-6-(2,6-dimethylphenyl)pyrimidin-2-yl]sulfamoyl]benzoic acid

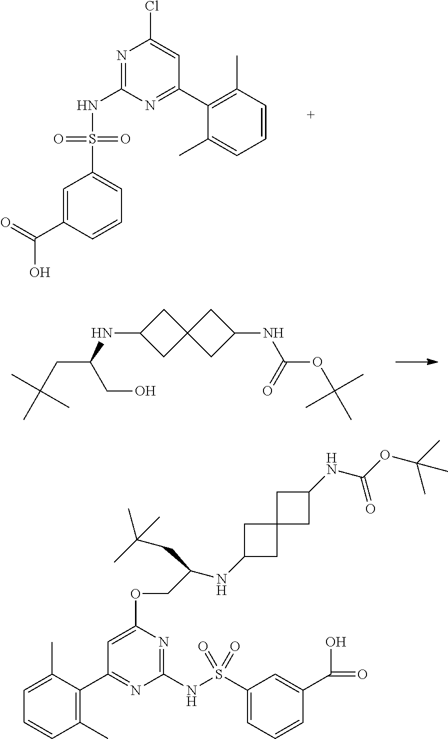 [SMILES: Cc1cccc(C)c1-c1cc(Cl)nc(NS(=O)(=O)c2cccc(C(=O)O)c2)n1 ; CC(C)(C)C[C@H](CO)NC1CC2(CC(NC(=O)OC(C)(C)C)C2)C1 ; Cc1cccc(C)c1-c1cc(OC[C@@H](CC(C)(C)C)NC2CC3(CC(NC(=O)OC(C)(C)C)C3)C2)nc(NS(=O)(=O)c2cccc(C(=O)O)c2)n1]

A solution of tert-butyl N-[6-[[(1R)-1-(hydroxymethyl)-3,3-dimethyl-butyl]amino]spiro[3.3]heptan-2-yl]carbamate (70.7 g, 207.6 mmol) and 3-[[4-chloro-6-(2,6-dimethylphenyl)pyrimidin-2-yl]sulfamoyl]benzoic acid (hydrochloride salt) (93.0 g, 202.2 mmol) in MeTHF (800 mL) was stirred for 10 min at ambient temperature. To the mixture was slowly added sodium tert-butoxide (100 g, 1.041 mol) using a water bath for cooling and keeping the reaction temperature <40° C. After the addition, the reaction mixture became a light orange slurry and was stirred at ambient temperature for 45 min. The reaction was warmed to 40° C. and stirred for 45 min. Sodium tert-butoxide (19.6 g, 203.9 mmol) was added and the reaction was stirred at 30° C. The reaction gradually cooled to ambient temperature and became a light orange slurry. The reaction was cooled with an ice-bath and the reaction quenched with the slow addition of HCl (800 mL of 2 M, 1.600 mol) and stirred for 5 min. The mixture was transferred to a separatory funnel using EtOAc. The organic phase was separated, and the aqueous phase was extracted with EtOAc (400 mL). The combined organic phases were washed with 500 mL of brine, dried over magnesium sulfate, filtered over Celite and concentrated in vacuo affording an orange foam. The crude product was used without further purification. 3-[[4-[(2R)-2-[[2-(tert-butoxycarbonylamino)spiro[3.3]heptan-6-yl]amino]-4,4-dimethyl-pentoxy]-6-(2,6-dimethylphenyl)pyrimidin-2-yl]sulfamoyl]benzoic acid (hydrochloride salt) (157 g, 100%) ESI-MS m/z calc. 721.3509, found 722.2 (M+1)⁺; Retention time: 1.23 minutes (LC method A).

### Step 3: tert-Butyl ((2S,4s,6S)-6-((R)-1⁶-(2,6-dimethylphenyl)-7-neopentyl-3,3-dioxido-5-oxo-9-oxa-3-thia-2,6-diaza-1(2,4)-pyrimidina-4(1,3)-benzenacyclononaphane-6-yl)spiro[3.3]heptan-2-yl)carbamate (Compound 89), and tert-butyl ((2R,4r,6R)-6-((R)-1⁶-(2,6-dimethylphenyl)-7-neopentyl-3,3-dioxido-5-oxo-9-oxa-3-thia-2,6-diaza-1(2,4)-pyrimidina-4(1,3)-benzenacyclononaphane-6-yl)spiro[3.3]heptan-2-yl)carbamate (Compound 90)

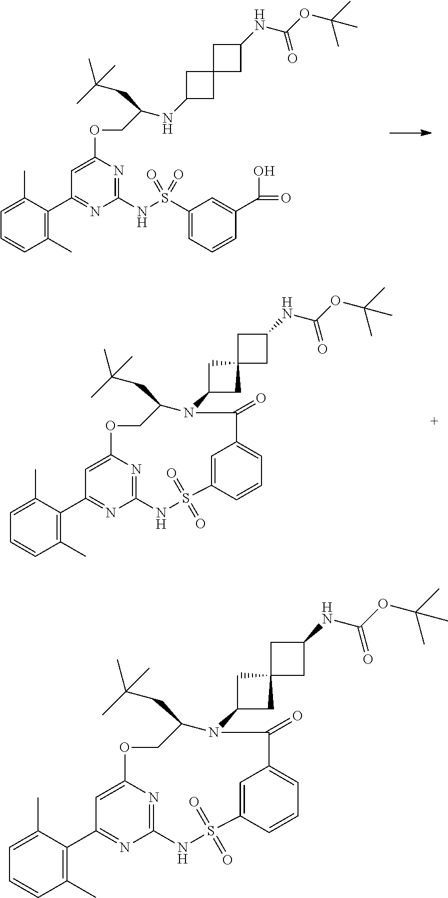

To a solution of 3-[[4-[(2R)-2-[[2-(tert-butoxycarbonylamino)spiro[3.3]heptan-6-yl]amino]-4,4-dimethyl-pentoxy]-6-(2,6-dimethylphenyl)pyrimidin-2-yl]sulfamoyl]benzoic acid (hydrochloride salt) (24.5 g, 32.31 mmol) in DMF (300 mL) at 0° C. was added DIEA (18 mL, 103.3 mmol) followed by the portionwise addition of HATU (18.4 g, 48.39 mmol). The cooling bath was removed, and the mixture stirred at ambient temperature for 24 h. The mixture was slowly poured into a solution of HCl (8.0 mL of 12 M, 96.00 mmol) in water (900 mL) and stirred at ambient temperature for 10 min. The tan slurry was filtered using a M frit. The precipitate was washed 3×with 50 mL of water and air dried for 18 h. The filter cake was dissolved in EtOAc (500 mL) and the water phase separated. The aqueous phase was extracted with 300 mL of EtOAc and the combined organic phases were concentrated in vacuo. The crude product was chromatographed on a 750 g column eluting with 10-100% EtOAc/hexanes (product eluted at 70% EtOAc). Impure product obtained from previous reactions was combined and chromatographed on a 750 g column eluting with 10-100% EtOAc/hexanes (product eluted at 70% EtOAc). Impure fractions were combined and chromatographed on a 450 g Reverse Phase column eluting with 50-100% ACN/Water to give tert-butyl N-{6-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(18),4(19),5,7,14,16-hexaen-12-yl]spiro[3.3]heptan-2-yl}carbamate (14.3 g, 63%). ¹H NMR (400 MHz, DMSO-d₆) δ 13.02 (s, 1H), 8.41 (s, 1H), 7.91 (s, 1H), 7.66 (s, 2H), 7.25 (t, J=7.6 Hz, 1H), 7.09 (dd, J=21.9, 7.8 Hz, 3H), 6.39 (s, 1H), 5.14-4.97 (m, 1H), 4.29 (d, J=6.4 Hz, 1H), 3.87 (dt, J=19.3, 8.9 Hz, 2H), 3.66 (s, 1H), 3.18-2.90 (m, 2H), 2.38 (d, J=8.5 Hz, 1H), 2.27 (d, J=38.2 Hz, 2H), 2.17-1.99 (m, 5H), 1.86 (d, J=30.6 Hz, 5H), 1.58 (dt, J=16.5, 8.6 Hz, 1H), 1.37 (s, 9H), 0.49 (d, J=3.9 Hz, 9H). ESI-MS m/z calc. 703.34033, found 704.4 (M+1)⁺; Retention time: 2.96 minutes (LC method A).

This product was combined with material from previous experiments affording a total of 62 g, which was subjected to chiral SFC separation using a LUX-CEL-4 column (2×25 cm) with a mobile phase of 35% methanol/CO₂ at 60 mL/min. Sample concentration was 20 mg/mL in methanol, with 4 mL injections, outlet pressure of 100 bar, and detection wavelength of 220 nm to give two products: Peak 1, tert-butyl ((2S,4s,6S)-6-((R)-1⁶-(2,6-dimethylphenyl)-7-neopentyl-3,3-dioxido-5-oxo-9-oxa-3-thia-2,6-diaza-1(2,4)-pyrimidina-4(1,3)-benzenacyclononaphane-6-yl)spiro[3.3]heptan-2-yl)carbamate (27.7 g, 88%), ¹H NMR (400 MHz, DMSO-d₆) δ 13.03 (s, 1H), 8.40 (s, 1H), 7.91 (s, 1H), 7.65 (s, 2H), 7.25 (t, J=7.7 Hz, 1H), 7.08 (dd, J=21.2, 7.8 Hz, 3H), 6.38 (s, 1H), 5.05 (dd, J=10.7, 4.3 Hz, 1H), 4.27 (t, J=11.3 Hz, 1H), 3.87 (tt, J=16.4, 8.4 Hz, 2H), 3.66 (s, 1H), 3.44 (qd, J=7.0, 5.1 Hz, 1H), 3.17 (d, J=5.2 Hz, 2H), 3.00 (dt, J=36.7, 9.6 Hz, 2H), 2.44-2.16 (m, 4H), 2.11 (s, 2H), 1.96 (t, J=9.9 Hz, 4H), 1.59 (dd, J=15.1, 8.3 Hz, 1H), 1.37 (s, 9H), 0.49 (s, 9H), ESI-MS m/z calc. 703.34033, found 704.4 (M+1)⁺; Retention time: 2.96 minutes (LC method A), and peak 2, tert-butyl ((2R,4r,6R)-6-((R)-1⁶-(2,6-dimethylphenyl)-7-neopentyl-3,3-dioxido-5-oxo-9-oxa-3-thia-2,6-diaza-1(2,4)-pyrimidina-4(1,3)-benzenacyclononaphane-6-yl)spiro[3.3]heptan-2-yl)carbamate (25.3 g, 81%), ¹H NMR (400 MHz, DMSO-d₆) δ 13.05 (s, 1H), 8.42 (s, 1H), 7.91 (s, 1H), 7.65 (s, 2H), 7.24 (d, J=7.9 Hz, 1H), 7.19-6.98 (m, 3H), 6.39 (s, 1H), 5.07 (dd, J=10.7, 4.4 Hz, 1H), 4.33-4.23 (m, 1H), 3.85 (ddd, J=32.0, 17.2, 8.9 Hz, 2H), 3.66 (s, 1H), 3.44 (qd, J=7.0, 5.2 Hz, 1H), 3.17 (d, J=5.3 Hz, 2H), 2.99 (dt, J=18.8, 9.7 Hz, 2H), 2.39 (d, J=10.9 Hz, 1H), 2.31-2.19 (m, 2H), 2.13 (s, 3H), 1.96-1.86 (m, 4H), 1.57 (dd, J=15.1, 8.3 Hz, 1H), 1.37 (s, 9H), 0.48 (s, 9H). ESI-MS m/z calc. 703.34033, found 704.3 (M+1)⁺; Retention

### Step 4: (R)-6-((2S,4s,6S)-6-Aminospiro[3.3]heptan-2-yl)-1⁶-(2,6-dimethylphenyl)-7-neopentyl-9-oxa-3-thia-2,6-diaza-1(2,4)-pyrimidina-4(1,3)-benzenacyclononaphan-5-one 3,3-dioxide

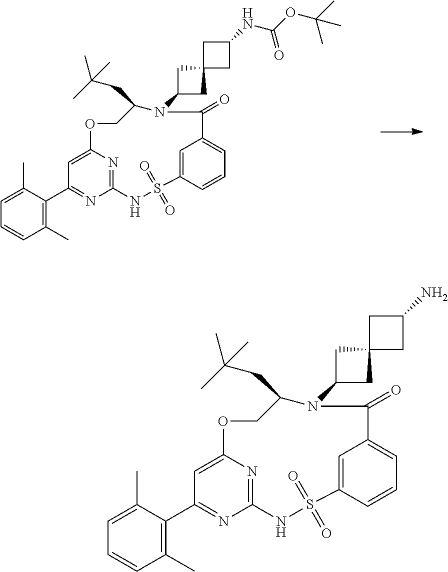

To a mixture of tert-butyl ((2S,4s,6S)-6-((R)-1⁶-(2,6-dimethylphenyl)-7-neopentyl-3,3-dioxido-5-oxo-9-oxa-3-thia-2,6-diaza-1(2,4)-pyrimidina-4(1,3)-benzenacyclononaphane-6-yl)spiro[3.3]heptan-2-yl)carbamate (68.9 g, 97.88 mmol) in MeOH (400 mL) was added HCl (100 mL of 4 M, 400.0 mmol) and the mixture stirred at ambient temperature for 18 h. The solvent was removed in vacuo. The off-white solid was slurried in MeTHF/DCM and the solvent removed in vacuo. The product was placed under high vac for 24 h and used in the next step without further purification. (R)-6-((2S,4s,6S)-6-Aminospiro[3.3]heptan-2-yl)-1⁶-(2,6-dimethylphenyl)-7-neopentyl-9-oxa-3-thia-2,6-diaza-1(2,4)-pyrimidina-4(1,3)-benzenacyclononaphan-5-one 3,3-dioxide (hydrochloride salt) (58.8 g, 94%)¹H NMR (400 MHz, DMSO-d₆) δ 8.42 (s, 1H), 8.18 (d, J=5.3 Hz, 3H), 7.92 (dt, J=7.1, 2.0 Hz, 1H), 7.67 (d, J=7.2 Hz, 2H), 7.26 (t, J=7.6 Hz, 1H), 7.12 (d, J=7.6 Hz, 2H), 6.41 (s, 3H), 5.07 (dd, J=10.7, 4.4 Hz, 1H), 4.28 (t, J=11.2 Hz, 1H), 3.94 (p, J=8.5 Hz, 1H), 3.67 (ddd, J=12.3, 7.9, 4.6 Hz, 1H), 3.14-2.95 (m, 2H), 2.48-2.34 (m, 2H), 2.34-2.15 (m, 4H), 1.99 (s, 6H), 1.59 (dd, J=15.2, 8.4 Hz, 1H), 1.36 (d, J=14.9 Hz, 1H), 0.49 (s, 9H). ESI-MS m/z calc. 603.2879, found 604.5 (M+1)⁺; Retention time: 1.32 minutes (LC method A).

### Step 5: Methyl ((2S,4s,6S)-6-((R)-1⁶-(2,6-dimethylphenyl)-7-neopentyl-3,3-dioxido-5-oxo-9-oxa-3-thia-2,6-diaza-1(2,4)-pyrimidina-4(1,3)-benzenacyclononaphane-6-yl)spiro[3.3]heptan-2-yl)carbamate (Compound 88)

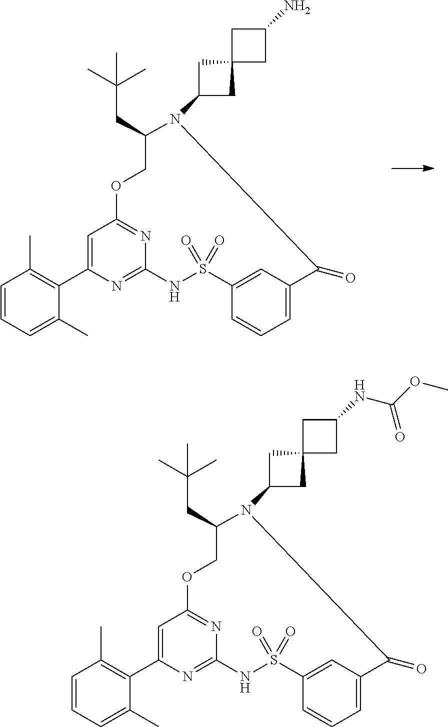

In a reaction vial, (R)-6-((2S,4s,6S)-6-aminospiro[3.3]heptan-2-yl)-1⁶-(2,6-dimethylphenyl)-7-neopentyl-9-oxa-3-thia-2,6-diaza-1(2,4)-pyrimidina-4(1,3)-benzenacyclononaphan-5-one 3,3-dioxide (hydrochloride salt) (3.5 g, 5.467 mmol) was mixed with diisopropylethylamine (3.14 mL, 18.03 mmol) in methylene chloride (70 mL). The reaction was cooled to −10° C. using an ice-salt bath then methyl chloroformate (465 μL, 6.018 mmol) was added. The reaction mixture was allowed to stir at −10° C. for 15 minutes and allowed to warm to rt. After stirring at rt for 1 h, the reaction mixture was evaporated to dryness then diluted with ethyl acetate then washed with 1N HCl (3×) and saturated NaCl solution. The organic layer was isolated, dried over anhydrous sodium sulfate, filtered, and evaporated to dryness. The crude material was purified by column chromatography on silica using 50-100% EtOAc/Hexanes gradient. The isolated solid was washed with hexanes then dried under high vacuum at 40° C. overnight. The product was isolated as a white powder. Methyl ((2S,4s,6S)-6-((R)-1⁶-(2,6-dimethylphenyl)-7-neopentyl-3,3-dioxido-5-oxo-9-oxa-3-thia-2,6-diaza-1(2,4)-pyrimidina-4(1,3)-benzenacyclononaphane-6-yl)spiro[3.3]heptan-2-yl)carbamate (2.753 g, 76%). ¹H NMR (400 MHz, DMSO-d₆) δ 8.40 (s, 1H), 7.91 (s, 1H), 7.66 (s, 2H), 7.36 (d, J=7.9 Hz, 1H), 7.26 (t, J=7.7 Hz, 1H), 7.11 (d, J=7.7 Hz, 2H), 6.40 (s, 1H), 5.06 (dd, J=10.7, 4.3 Hz, 1H), 4.28 (t, J=11.2 Hz, 1H), 3.88 (hept, J=10.5, 9.7 Hz, 3H), 3.67 (d, J=7.7 Hz, 1H), 3.32 (s, 2H), 3.06 (t, J=9.7 Hz, 1H), 2.98 (t, J=10.0 Hz, 1H), 2.42 (d, J=6.1 Hz, 1H), 2.35 (d, J=10.0 Hz, 1H), 2.26 (p, J=6.3 Hz, 2H), 2.17-2.07 (m, 3H), 1.98 (q, J=9.3, 7.8 Hz, 4H), 1.93-1.88 (m, 2H), 1.60 (dd, J=15.2, 8.3 Hz, 1H), 1.36 (d, J=15.0 Hz, 1H), 0.49 (s, 9H). ESI-MS m/z calc. 661.2934, found 662.4 (M+1)⁺; Retention time: 2.64 minutes (LC method I).

### Example 71: Preparation of Compound 91

### Step 1: tert-Butyl N-[2-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]spiro[3.3]heptan-6-yl]carbamate

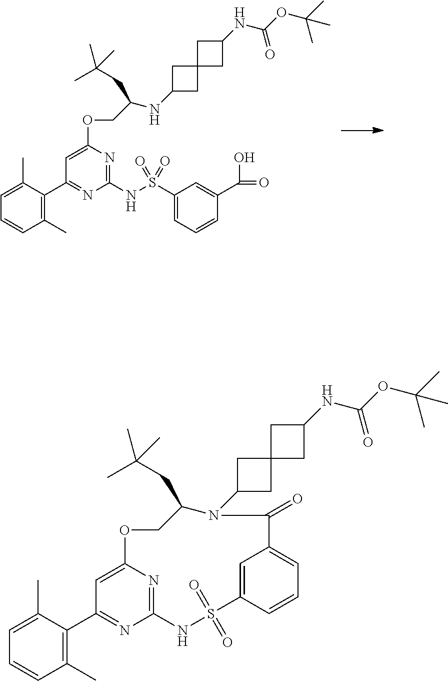 [SMILES: Cc1cccc(C)c1-c1cc(OC[C@@H](CC(C)(C)C)NC2CC3(CC(NC(=O)OC(C)(C)C)C3)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CC(NC(=O)OC(C)(C)C)C3)C1)[C@H](CC(C)(C)C)CO2]

To a solution of 3-[[4-[(2R)-2-[[2-(tert-butoxycarbonylamino)spiro[3.3]heptan-6-yl]amino]-4,4-dimethyl-pentoxy]-6-(2,6-dimethylphenyl)pyrimidin-2-yl]sulfamoyl]benzoic acid (hydrochloride salt) (45.77 g, 60.35 mmol) in DMF (500 mL) at 0° C. was added DIEA (32 mL, 183.7 mmol) followed by the portionwise addition of HATU (34.4 g, 90.47 mmol). The cooling bath was removed, and the mixture stirred at ambient temperature for 36 hours. The mixture was slowly poured into a solution of HCl (15 mL of 12 M, 180.0 mmol) in water (1.5 L) and stirred at ambient temperature for 10 min. The tan slurry was filtered using an M frit. The precipitate was washed 3×with 50 mL of water and air dried for 12 h. The filter cake was dissolved in EtOAc (500 mL) and the water phase separated. The aqueous phase was extracted with 300 mL of EtOAc and the combined organic phases were concentrated in vacuo affording a dark amber oil. The crude product was chromatographed on a 750 g column eluting with 10-100% EtOAc/hexanes (product eluted at 70% EtOAc). The product was combined with material from several smaller batches to give tert-butyl N-[2-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]spiro[3.3]heptan-6-yl]carbamate (34 g, 80%) ESI-MS m z calc. 703.34033, found 704.2 (M+1)⁺; Retention time: 3.05 minutes (LC method I).

### Step 2: (11R)-12-(6-Aminospiro[3.3]heptan-2-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

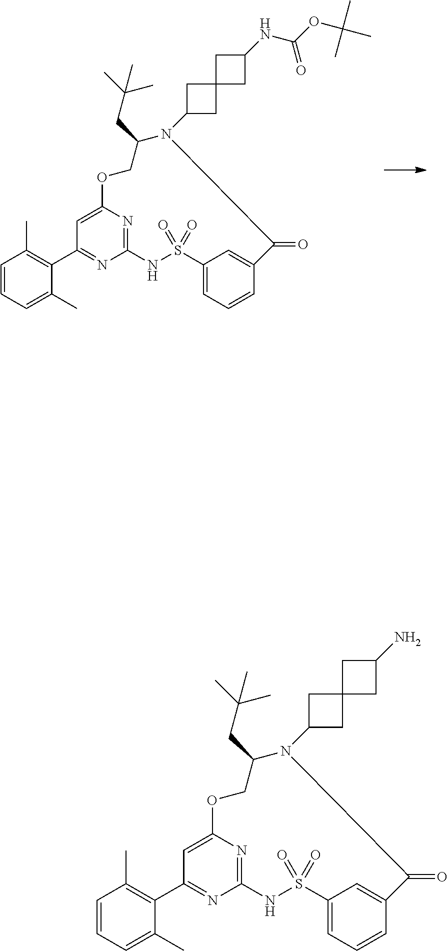 [SMILES: Cc1cccc(C)c1-c1cc2nc(n1)NS(=O)(=O)c1cccc(c1)C(=O)N(C1CC3(CC(NC(=O)OC(C)(C)C)C3)C1)[C@H](CC(C)(C)C)CO2 ; Cc1cccc(C)c1-c1cc2nc(n1)NS(=O)(=O)c1cccc(c1)C(=O)N(C1CC3(CC(N)C3)C1)[C@H](CC(C)(C)C)CO2]

In a reaction vial, tert-butyl N-[2-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]spiro[3.3]heptan-6-yl]carbamate (500 mg, 0.7103 mmol) was dissolved in methylene chloride (2 mL) along with HCl (1.776 mL of 4 M, 7.104 mmol)(4M in Dioxane). The reaction mixture was stirred at rt for 1.5 h. then evaporated to dryness. The solid material was slurried in a mixture of 50% ethyl acetate/hexanes and filtered. The product was recovered as a white solid (HCl salt). (11R)-12-(6-aminospiro[3.3]heptan-2-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (434.7 mg, 96%) ESI-MS m/z calc. 603.2879, found 604.6 (M+1)⁺; Retention time: 1.3 minutes (LC method A).

### Step 3: Methyl ((2R,4r,6R)-6-((R)-1⁶-(2,6-dimethylphenyl)-7-neopentyl-3,3-dioxido-5-oxo-9-oxa-3-thia-2,6-diaza-1(2,4)-pyrimidina-4(1,3)-benzenacyclononaphane-6-yl)spiro[3.3]heptan-2-yl)carbamate (Compound 91)

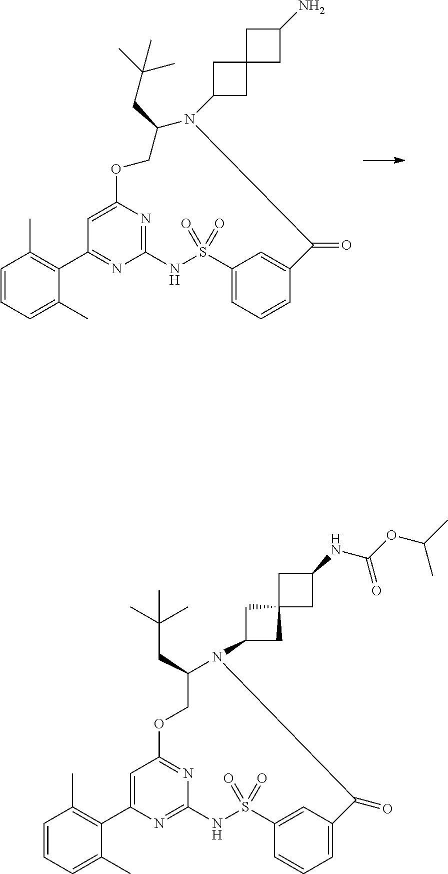

In a reaction vial, (11R)-12-(6-aminospiro[3.3]heptan-2-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (102.4 mg, 0.1599 mmol) was mixed with diisopropylethylamine (68.2 mg, 0.5277 mmol) in methylene chloride (2 mL). The reaction was cooled to −10° C. using an ice-salt bath then methyl chloroformate (15.87 mg, 0.1679 mmol) was added. The reaction mixture was allowed to stir at −10° C. for 15 minutes, warmed to rt, concentrated to about half the volume. The reaction mixture was diluted with ethyl acetate then washed with 1N HCl (3×) and saturated NaCl solution. The organic layer was isolated, dried over anhydrous sodium sulfate, filtered, and evaporated to dryness. The crude material was purified using a normal phase SFC-MS method using a LUX-3 column (250×21.2 mm, 5 m particle size) sold by Phenomenex (pn: 00G-4493-P0-AX), and a dual gradient run from 10-40% mobile phase B over 14.5 minutes (includes 40-80% mobile phase rinsate). Mobile phase A=CO₂. Mobile phase B=MeOH (20 mM NH3). Flow rate=10-40% MeOH [20 mM NH3] 60 mL/min, 40-80% MeOH [20 mM NH3] 60 mL/min. injection volume=variable, and column temperature=40° C., to give two isomers, Peak 1 and Peak 2. Peak 2 was methyl ((2R,4r,6R)-6-((R)-16-(2,6-dimethylphenyl)-7-neopentyl-3,3-dioxido-5-oxo-9-oxa-3-thia-2,6-diaza-1(2,4)-pyrimidina-4(1,3)-benzenacyclononaphane-6-yl)spiro[3.3]heptan-2-yl)carbamate (30.1 mg, 53%)¹H NMR (400 MHz, Methanol-d₄) δ 8.49 (s, 1H), 7.96-7.89 (m, 1H), 7.50 (d, J=6.0 Hz, 2H), 7.05 (t, J=7.6 Hz, 1H), 6.94 (d, J=7.7 Hz, 2H), 5.91 (s, 1H), 5.18 (dd, J=10.8, 4.4 Hz, 1H), 3.97 (t, J=11.1 Hz, 1H), 3.90-3.78 (m, 3H), 3.51 (s, 3H), 3.03 (dt, J=18.8, 9.8 Hz, 2H), 2.44 (t, J=8.7 Hz, 1H), 2.31 (q, J=8.0, 4.5 Hz, 2H), 2.20-2.06 (m, 2H), 1.93 (q, J=10.1 Hz, 4H), 1.83 (s, 1H), 1.52 (dd, J=15.0, 7.9 Hz, 2H), 1.44 (d, J=15.0 Hz, 1H), 1.19 (s, 2H), 0.79 (d, J=7.2 Hz, 1H), 0.45 (s, 8H). ESI-MS m z calc. 661.2934, found 662.3 (M+1)⁺; Retention time: 1.58 minutes (LC method 1A).

### Example 72: Preparation of Compound 92

### Step 1: Isopropyl ((2S,4s,6S)-6-((R)-1⁶-(2,6-dimethylphenyl)-7-neopentyl-3,3-dioxido-5-oxo-9-oxa-3-thia-2,6-diaza-1(2,4)-pyrimidina-4(1,3)-benzenacyclononaphane-6-yl)spiro[3.3]heptan-2-yl)carbamate (Compound 92)

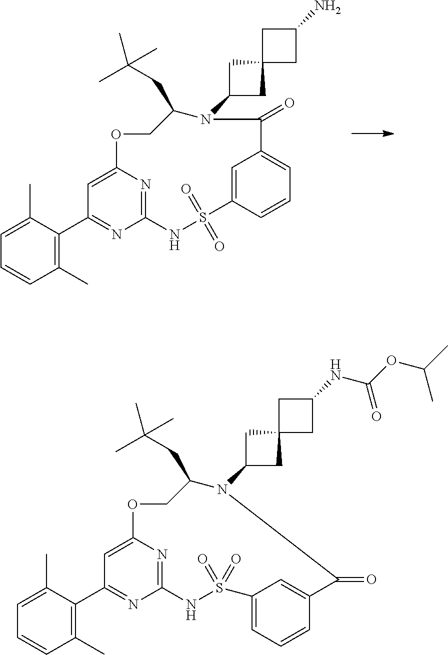

Using an overhead stirrer, to a slurry of (R)-6-((2S,4s,6S)-6-aminospiro[3.3]heptan-2-yl)-16-(2,6-dimethylphenyl)-7-neopentyl-9-oxa-3-thia-2,6-diaza-1(2,4)-pyrimidina-4(1,3)-benzenacyclononaphan-5-one 3,3-dioxide (hydrochloride salt) (58.8 g, 91.84 mmol) in MeTHF (400 mL) and DCM (100 mL) was added DIEA (40 mL, 229.6 mmol). To the mixture was added isopropyl chloroformate (115 mL of 1 M in toluene, 115.0 mmol) portionwise over 10 min. A slight exotherm was observed. The mixture was stirred at ambient temperature for 4 h. Additional DIEA (10 mL, 57.41 mmol) followed by isopropyl chloroformate (40 mL of 1 M (in toluene), 40.00 mmol) were added and the mixture was stirred at ambient temperature for a total of 18 h. The mixture was diluted with EtOAc (500 mL) and washed with HCl (500 mL of 1 M, 500.0 mmol). The aqueous phase was separated and extracted with EtOAc (500 mL). The combined organic phases were washed with brine, dried over magnesium sulfate, filtered and concentrated in vacuo. The crude product was chromatographed on a 750 g silica gel column eluting with 10-100% EtOAc/hexanes. The pure fractions were combined and concentrated in vacuo affording a foam. The foam was dried under high vac for 4 days at ambient temperature. The material was further dried under high vac at 45° C. for 3 days to give isopropyl ((2S,4s,6S)-6-((R)-1⁶-(2,6-dimethylphenyl)-7-neopentyl-3,3-dioxido-5-oxo-9-oxa-3-thia-2,6-diaza-1(2,4)-pyrimidina-4(1,3)-benzenacyclononaphane-6-yl)spiro[3.3]heptan-2-yl)carbamate (55.42 g, 87%) ¹H NMR (400 MHz, DMSO-d₆) δ 13.04 (s, 1H), 8.41 (s, 1H), 7.91 (d, J=6.2 Hz, 1H), 7.82-7.53 (m, 2H), 7.25 (t, J=6.9 Hz, 2H), 7.11 (d, J=7.6 Hz, 2H), 6.40 (s, 1H), 5.06 (dd, J=10.7, 4.4 Hz, 1H), 4.72 (hept, J=6.3 Hz, 1H), 4.28 (t, J=11.2 Hz, 1H), 3.88 (dp, J=16.0, 8.5 Hz, 2H), 3.74-3.58 (m, 1H), 3.06 (t, J=9.6 Hz, 1H), 2.97 (t, J=9.9 Hz, 1H), 2.48-2.18 (m, 3H), 2.18-2.03 (m, 3H), 2.02-1.73 (m, 6H), 1.60 (dd, J=15.2, 8.3 Hz, 1H), 1.36 (d, J=14.9 Hz, 1H), 1.16 (t, J=6.6 Hz, 6H), 0.49 (s, 9H). ESI-MS m/z calc. 689.3247, found 690.4 (M+1)⁺; Retention time: 2.82 minutes, LC method I.

### Example 73: Preparation of Compound 93

### Step 1: Isopropyl ((2R,4r,6R)-6-((R)-1⁶-(2,6-dimethylphenyl)-7-neopentyl-3,3-dioxido-5-oxo-9-oxa-3-thia-2,6-diaza-1(2,4)-pyrimidina-4(1,3)-benzenacyclononaphane-6-yl)spiro[3.3]heptan-2-yl)carbamate (Compound 93)

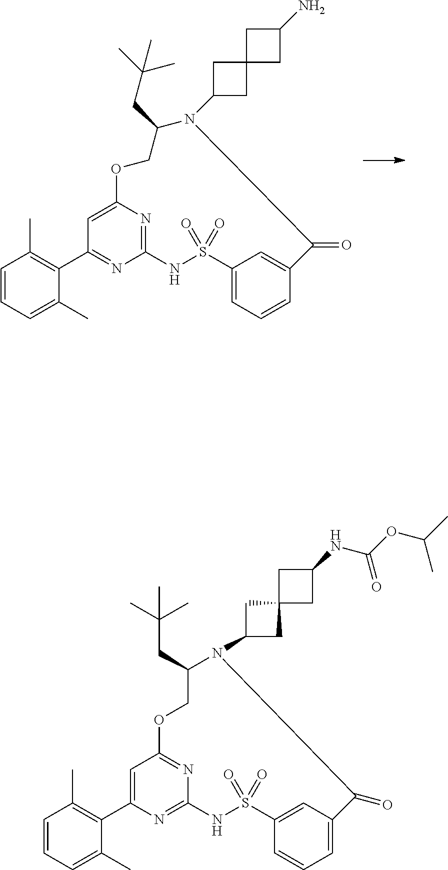

In a reaction vial, (11R)-12-(6-aminospiro[3.3]heptan-2-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (110.06 mg, 0.1719 mmol) was mixed with DIEA (98.8 μL, 0.5672 mmol) in dichloromethane (2.2 mL). The reaction was cooled to −10° C. using an ice-salt bath then isopropyl chloroformate (90.25 μL of 2 M in toluene, 0.1805 mmol) was added. The reaction mixture was allowed to warm up to room temperature and stirred for 1 hour. More DIEA (98.8 μL, 0.5672 mmol) and isopropyl chloroformate (22.12 mg, 2M in toluene 0.1805 mmol) were added. The reaction was allowed to stir at rt for an additional 15 min then evaporated to dryness. The reaction mixture was diluted with ethyl acetate then washed with 1N HCl (3×) and saturated NaCl solution. The organic layer was isolated, dried over anhydrous sodium sulfate, filtered, and evaporated to dryness. The crude material was purified using a normal phase SFC-MS method using a LUX-3 column (250×21.2 mm, 5 m particle size) sold by Phenomenex (pn: 00G-4493-P0-AX), and a dual gradient run from 10-40% mobile phase B over 14.5 minutes (includes 40-80% mobile phase rinsate). Mobile phase A=CO₂. Mobile phase B=MeOH (20 mM NH3). Flow rate=10-40% MeOH [20 mM NH3] 60 mL/min, 40-80% MeOH [20 mM NH3] 60 mL/min. injection volume=variable, and column temperature=40° C., to give two isomers peak 1 and peak 2. Peak 2 was isopropyl ((2R,4r,6R)-6-((R)-1⁶-(2,6-dimethylphenyl)-7-neopentyl-3,3-dioxido-5-oxo-9-oxa-3-thia-2,6-diaza-1(2,4)-pyrimidina-4(1,3)-benzenacyclononaphane-6-yl)spiro[3.3]heptan-2-yl)carbamate (25.6 mg, 41%)¹H NMR (400 MHz, Methanol-d₄) δ 8.48 (s, 1H), 7.92 (d, J=7.0 Hz, 1H), 7.60-7.50 (m, 2H), 7.12 (t, J=7.7 Hz, 1H), 6.99 (d, J=7.6 Hz, 2H), 6.05 (s, 1H), 5.18 (dd, J=10.8, 4.3 Hz, 1H), 4.48 (s, 1H), 4.08 (t, J=11.2 Hz, 1H), 3.87 (q, J=8.8 Hz, 2H), 3.78-3.73 (m, 1H), 3.02 (dt, J=18.9, 9.9 Hz, 2H), 2.48-2.39 (m, 1H), 2.35-2.28 (m, 2H), 2.18-2.14 (m, 1H), 2.11-1.75 (m, 9H), 1.55 (dd, J=15.2, 8.2 Hz, 1H), 1.42 (d, J=15.1 Hz, 1H), 1.11 (d, J=6.2 Hz, 6H), 0.46 (s, 9H). ESI-MS m/z calc. 689.3247, found 690.3 (M+1)⁺; Retention time: 1.56 minutes (LC method 1A).

### Example 74: Preparation of Compound 94

### Step 1: Methyl ((2R,4r,6R)-6-((R)-1⁶-(2,6-dimethylphenyl)-7-neopentyl-3,3-dioxido-5-oxo-9-oxa-3-thia-2,6-diaza-1(2,4)-pyrimidina-4(1,3)-benzenacyclononaphane-6-yl)spiro[3.3]heptan-2-yl)(methyl)carbamate (Compound 94)

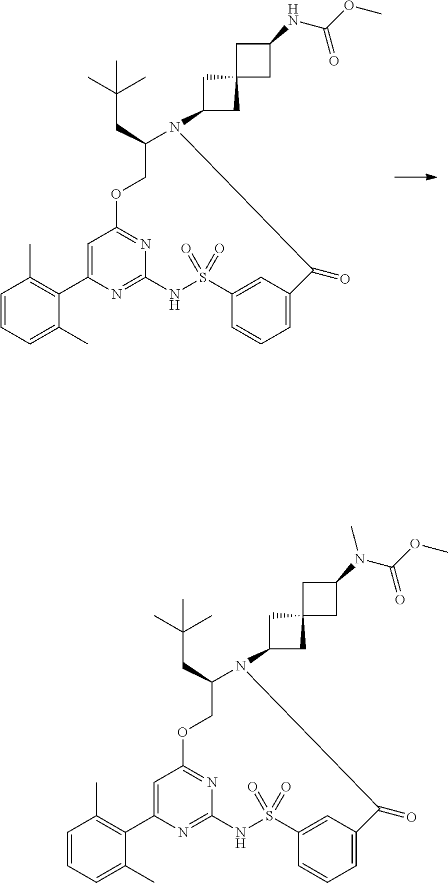

In a reaction vial, methyl ((2R,4r,6R)-6-((R)-1⁶-(2,6-dimethylphenyl)-7-neopentyl-3,3-dioxido-5-oxo-9-oxa-3-thia-2,6-diaza-1(2,4)-pyrimidina-4(1,3)-benzenacyclononaphane-6-yl)spiro[3.3]heptan-2-yl)carbamate (61.7 mg, 0.09323 mmol) was dissolved in THE (1.9 mL) and cooled to ° C. To the reaction, sodium hydride (18.65 mg of 60% w/w, 0.4663 mmol) was added and stirring was continued at 0° C. for 20 min. To the reaction mixture, iodomethane (26.5 mg, 0.1867 mmol) was added and the reaction was allowed to warm to rt. The reaction was stirred at rt for 1 h. then heated at 40° C. for 30 min. The reaction temperature was then raised to 60° C. and heated for 4 h. The reaction was quenched with 1N HCl and extracted with ethyl acetate. The crude material was purified by column chromatography on silica using 30-70% ethyl acetate/hexanes gradient to give methyl ((2R,4r,6R)-6-((R)-1⁶-(2,6-dimethylphenyl)-7-neopentyl-3,3-dioxido-5-oxo-9-oxa-3-thia-2,6-diaza-1(2,4)-pyrimidina-4(1,3)-benzenacyclononaphane-6-yl)spiro[3.3]heptan-2-yl)(methyl)carbamate (17.4 mg, 28%). ¹H NMR (400 MHz, Methanol-d₄) δ 8.46 (s, 1H), 7.92 (d, J=7.5 Hz, 1H), 7.61 (t, J=6.8 Hz, 1H), 7.57 (d, J=7.6 Hz, 1H), 7.17 (t, J=7.6 Hz, 1H), 7.03 (d, J=7.6 Hz, 2H), 6.17 (s, 1H), 5.18 (dd, J=10.8, 4.3 Hz, 1H), 4.13 (t, J=11.2 Hz, 1H), 3.89 (p, J=8.7 Hz, 1H), 3.72 (ddd, J=12.2, 8.3, 4.4 Hz, 1H), 3.57 (s, 3H), 3.11 (t, J=9.7 Hz, 1H), 2.99 (t, J=10.0 Hz, 1H), 2.75 (s, 3H), 2.45-2.32 (m, 1H), 2.35-2.24 (m, 1H), 2.22-2.11 (m, 5H), 2.08-1.78 (m, 6H), 1.60 (dd, J=15.3, 8.2 Hz, 1H), 1.41 (d, J=15.1 Hz, 1H), 1.23-1.10 (m, 1H), 0.49 (s, 9H). ESI-MS m z calc. 675.3091, found 676.5 (M+1)⁺; Retention time: 2.0 minutes (LC method A).

### Example 75: Preparation of Compound 95

### Step 1: (11R)-6-(2,6-Dimethylphenyl)-11-(2,2-dimethylpropyl)-3-(methoxymethyl)-2,2-dioxo-12-(2-oxospiro[3.3]heptan-6-yl)-9-oxa-2λ⁶-thia-3,5,12,19-tetrazatricyclo[12.3.1.14,8]nonadeca-1(18),4,6,8(19),14,16-hexaen-13-one

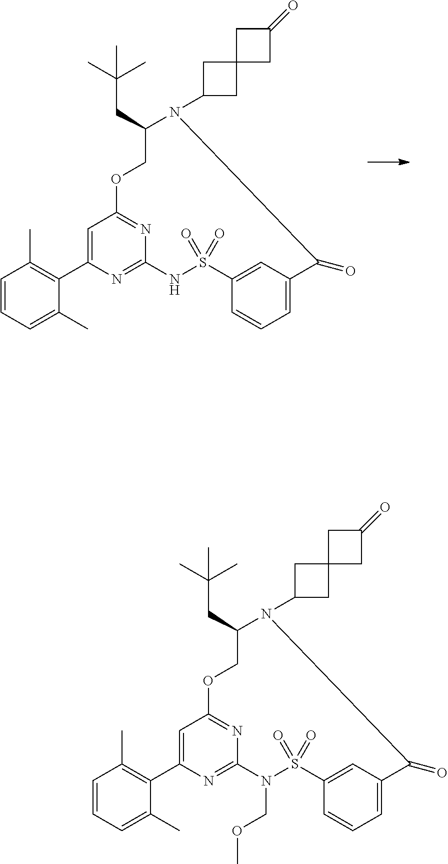 [SMILES: Cc1cccc(C)c1-c1cc2nc(n1)NS(=O)(=O)c1cccc(c1)C(=O)N(C1CC3(CC(=O)C3)C1)[C@H](CC(C)(C)C)CO2 ; COCN1c2nc(cc(-c3c(C)cccc3C)n2)OC[C@@H](CC(C)(C)C)N(C2CC3(CC(=O)C3)C2)C(=O)c2cccc(c2)S1(=O)=O]

In a reaction vial, (11R)-6-(2,6-dimethylphenyl)-11-(2,2-dimethylpropyl)-2,2-dioxo-12-(2-oxospiro[3.3]heptan-6-yl)-9-oxa-2λ⁶-thia-3,5,12,19-tetrazatricyclo[12.3.1.14,8]nonadeca-1(18),4,6,8(19),14,16-hexaen-13-one (265 mg, 0.4309 mmol) was dissolved in acetonitrile (2.2 mL) along with potassium carbonate (95.3 mg, 0.6896 mmol). To the reaction mixture, chloro(methoxy)methane (36 μL, 0.4740 mmol) was added dropwise and the reaction was stirred at rt overnight. The reaction was evaporated to dryness and partitioned between ethyl acetate/water. The organic layer was isolated, dried over anhydrous sodium sulfate, filtered, and evaporated to dryness. The crude material was purified by column chromatography on silica using 30-100% EtOAc/Hexanes gradient to give (11R)-6-(2,6-Dimethylphenyl)-11-(2,2-dimethylpropyl)-3-(methoxymethyl)-2,2-dioxo-12-(2-oxospiro[3.3]heptan-6-yl)-9-oxa-2λ⁶-thia-3,5,12,19-tetrazatricyclo[12.3.1.14,8]nonadeca-1(18),4,6,8(19),14,16-hexaen-13-one (83.3 mg, 30%). ESI-MS m/z calc. 646.28253, found 647.4 (M+1)⁺; Retention time: 1.93 minutes; LC method A.

### Step 2: N-[6-[(11R)-6-(2,6-Dimethylphenyl)-11-(2,2-dimethylpropyl)-3-(methoxymethyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro [3.3]heptan-2-ylidene]-2-methyl-propane-2-sulfinamide

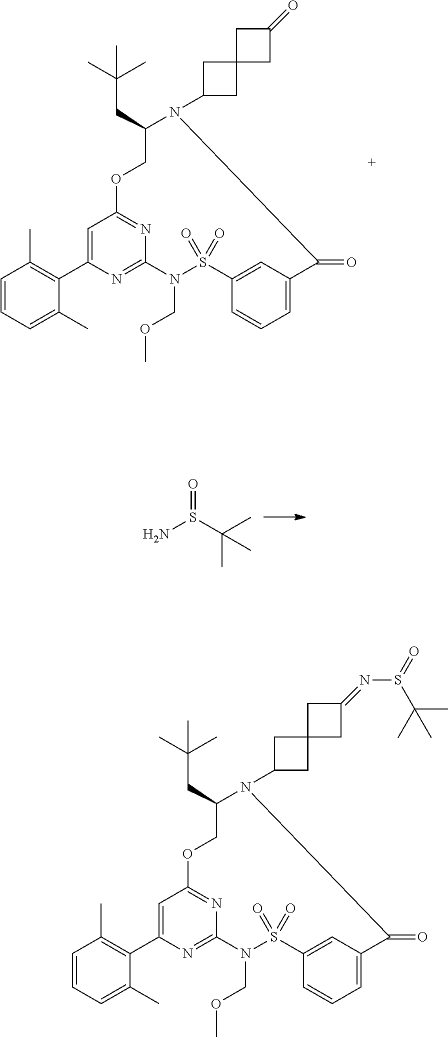

In a reaction vial, (11R)-6-(2,6-dimethylphenyl)-11-(2,2-dimethylpropyl)-3-(methoxymethyl)-2,2-dioxo-12-(2-oxospiro[3.3]heptan-6-yl)-9-oxa-2λ⁶-thia-3,5,12,19-tetrazatricyclo[12.3.1.14,8]nonadeca-1(18),4,6,8(19),14,16-hexaen-13-one (111 mg, 0.1716 mmol) was dissolved in tetrahydrofuran (0.5 mL) along with 2-methylpropane-2-sulfinamide (23 mg, 0.1898 mmol) (racemic). To the reaction, titanium(IV) ethoxide (73 μL, 0.3482 mmol) was added and the reaction was heated at 55° C. overnight. The reaction was cooled to rt and quenched with a saturated ammonium chloride solution. The reaction mixture was filtered and the solid was washed with THF. The filtrate was collected and evaporated to dryness. The crude material was purified by column chromatography on silica using 30-100% EtOAc/DCM gradient to give N-[6-[(11R)-6-(2,6-dimethylphenyl)-11-(2,2-dimethylpropyl)-3-(methoxymethyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-2-ylidene]-2-methyl-propane-2-sulfinamide (1:1 mixture of isomers, 100.4 mg, 78%) ESI-MS m/z calc. 749.32806, found 750.5 (M+1)⁺; Retention time: 2.05 and 2.37 minutes (LC method A).

### Step 3: (11R)-12-(2-Amino-2-methyl-spiro[3.3]heptan-6-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4,6,8(19),14,16-hexaen-13-one, diastereomer 1, and (11R)-12-(2-amino-2-methyl-spiro[3.3]heptan-6-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4,6,8(19),14,16-hexaen-13-one, diastereomer 2

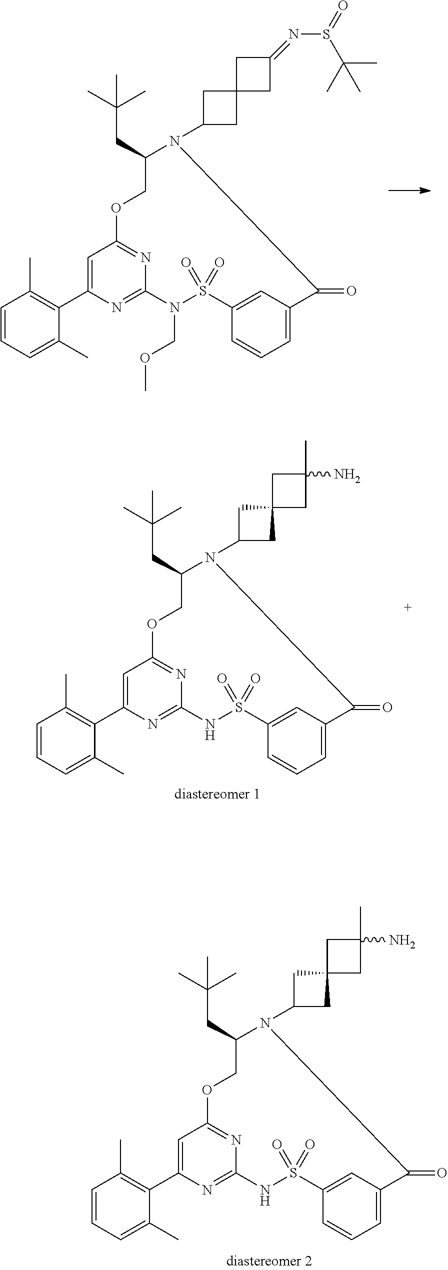

In a reaction vial, N-[6-[(11R)-6-(2,6-dimethylphenyl)-11-(2,2-dimethylpropyl)-3-(methoxymethyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-2-ylidene]-2-methyl-propane-2-sulfinamide (100 mg, 0.1333 mmol) was dissolved in Toluene (2.86 mL) and cooled to −78° C. To the reaction, trimethyl aluminum (147.6 μL of 2 M, 0.2952 mmol) was added slowly. After stirring the reaction at −78° C. for 20 min., methyllithium (365.9 μL of 1.6 M, 0.5854 mmol) was added slowly and stirring was continued at −78° C. for 1 h. The reaction was allowed to slowly warm to rt and stirred at that temperature for 15 min. then quenched with water (100 μL). The reaction mixture was then evaporated to dryness and the residue was dissolved in methanol (7.2 mL) along with HCl (99.98 μL of 4 M, 0.3999 mmol). The reaction mixture was stirred at rt overnight then purified by preparative HPLC using 20-100% ACN/water gradient. The individual isomers were isolated as two separate peaks: Diastereomer 1 (11R)-12-(2-amino-2-methyl-spiro[3.3]heptan-6-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4,6,8(19),14,16-hexaen-13-one (2.3 mg, 5%), ESI-MS m/z calc. 617.3036, found 618.6 (M+1)⁺; Retention time: 1.33 minutes (LC method A); and diastereomer 2 (11R)-12-(2-amino-2-methyl-spiro[3.3]heptan-6-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4,6,8(19),14,16-hexaen-13-one (2 mg, 5%), ESI-MS m/z calc. 617.3036, found 618.7 (M+1)⁺; Retention time: 1.41 minutes (LC method A).

### Step 4: Methyl N-[6-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]-2-methyl-spiro[3.3]heptan-2-yl]carbamate (Compound 95)

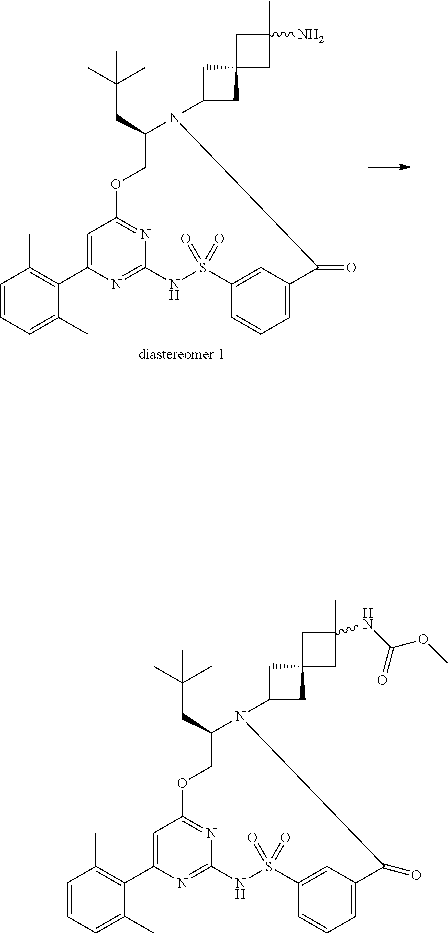

In a reaction vial, (11R)-12-(2-amino-2-methyl-spiro[3.3]heptan-6-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4,6,8(19),14,16-hexaen-13-one (2.3 mg, 0.003574 mmol)(diastereomer 1) was dissolved in DCM (250 μL) with DIEA (2.1 μL, 0.01206 mmol). The reaction mixture was cooled to 0° C. then methyl chloroformate (0.31 μL, 0.004012 mmol) was added. Stirring was continued at 0° C. for 1 h. then at rt for 1 h. The reaction mixture was diluted with ethyl acetate and washed with 1N HCl (3×) followed by saturated NaCl solution. The organic layer was isolated dried over anhydrous sodium sulfate, filtered, and evaporated to dryness. The residue was purified by preparative TLC using 5% MeOH/DCM as the eluent. The product was collected after evaporation of the solvents to give methyl N-[6-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]-2-methyl-spiro[3.3]heptan-2-yl]carbamate (0.9 mg, 34%), ESI-MS m/z calc. 675.3091, found 676.5 (M+1)⁺; Retention time: 1.92 minutes (LC method A).

### Example 76: Preparation of Compound 96

### Step 1: Methyl N-[6-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]-2-methyl-spiro[3.3]heptan-2-yl]carbamate (Compound 96)

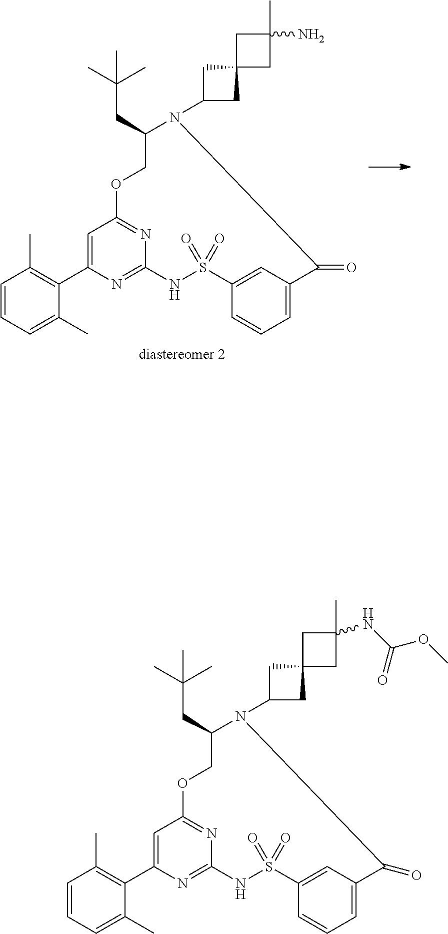

In a reaction vial, (11R)-12-(2-amino-2-methyl-spiro[3.3]heptan-6-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4,6,8(19),14,16-hexaen-13-one (2 mg, 0.003108 mmol) (diastereomer 2) was dissolved in DCM (250 μL) with diisopropylethylamine (1.8 μL, 0.01033 mmol). The reaction mixture was cooled to 0° C. then methyl chloroformate (0.27 μL, 0.003494 mmol) was added. Stirring was continued at 0° C. for 1 h. then at rt for 1 h. The reaction mixture was diluted with ethyl acetate and washed with 1N HCl (3×) followed by saturated NaCl solution. The organic layer was isolated dried over anhydrous sodium sulfate, filtered, and evaporated to dryness. The residue was purified by preparative TLC using 5% MeOH/DCM as the eluent. The product was collected after evaporation of the solvents to give methyl N-[6-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]-2-methyl-spiro[3.3]heptan-2-yl]carbamate (0.9 mg, 39%), ESI-MS m/z calc. 675.3091, found 676.5 (M+1)⁺; Retention time: 1.97 minutes (LC method A).

### Example 77: Preparation of Compound 97

### Step 1: (2R)-4-Methyl-2-[(1-methylpyrazol-4-yl)amino]pentan-1-ol

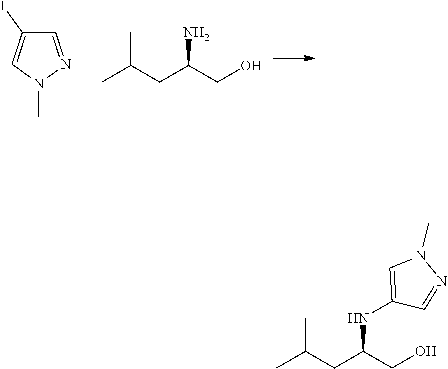

4-iodo-1-methyl-pyrazole (100 mg, 0.4808 mmol) was combined with CuI (approximately 9.157 mg, 0.04808 mmol), (2S)-pyrrolidine-2-carboxylic acid (approximately 11.07 mg, 0.09616 mmol), and cesium carbonate (approximately 469.8 mg, 1.442 mmol) in DMSO (0.5 mL) in a nitrogen-purged screwcap vial, and (2R)-2-amino-4-methyl-pentan-1-ol (approximately 73.24 mg, 76.29 μL, 0.6250 mmol) was added. The reaction mixture was heated to 80° C. (100° C. for 1-A2) for 16 hours. The reaction mixture was then diluted with methanol, filtered twice, and purified by reverse phase HPLC (1-30ACN in water, HCl modifier, 15 min run) to give (2R)-4-methyl-2-[(1-methylpyrazol-4-yl)amino]pentan-1-ol (17 mg, 18%) ESI-MS m/z calc. 197.15282, found 198.3 (M+1)⁺; Retention time: 0.25 minutes; LC method D.

### Step 2: 3-[[4-(2,6-Dimethylphenyl)-6-[(2R)-4-methyl-2-[(1-methylpyrazol-4-yl)amino]pentoxy]pyrimidin-2-yl]sulfamoyl]benzoic acid

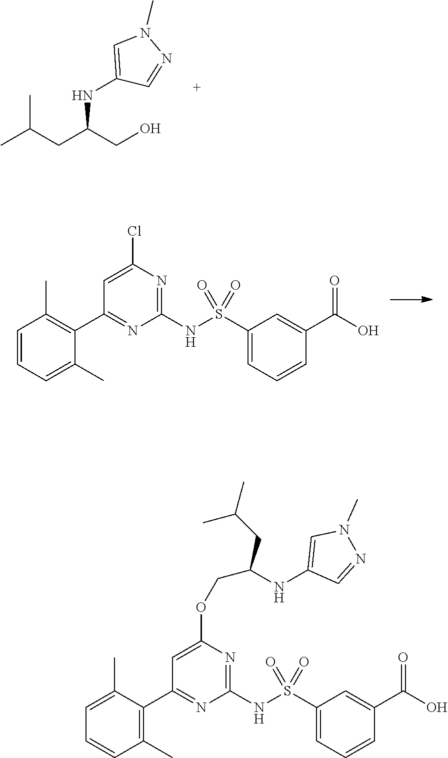 [SMILES: CC(C)C[C@H](CO)Nc1cnn(C)c1 ; Cc1cccc(C)c1-c1cc(Cl)nc(NS(=O)(=O)c2cccc(C(=O)O)c2)n1 ; Cc1cccc(C)c1-c1cc(OC[C@@H](CC(C)C)Nc2cnn(C)c2)nc(NS(=O)(=O)c2cccc(C(=O)O)c2)n1]

(2R)-4-Methyl-2-[(1-methylpyrazol-4-yl)amino]pentan-1-ol (60 mg, 0.3041 mmol) was combined with 3-[[4-chloro-6-(2,6-dimethylphenyl)pyrimidin-2-yl]sulfamoyl]benzoic acid (100 mg, 0.2393 mmol) in THE (0.5 mL) and stirred until the solids had mostly dissolved/become a suspension. Sodium tert-butoxide (140 mg, 1.457 mmol) was added and the reaction briefly became slightly warm. Stirring was continued for 15 minutes with no external heating. The reaction mixture was then partitioned between 1M HCl and ethyl acetate. The layers were separated, and the aqueous was extracted an additional 4×with ethyl acetate. The combined organics were washed with brine, dried over sodium sulfate, and concentrated. The resulting crude material was dissolved in 1:1 DMSO/methanol, filtered, and purified by reverse phase HPLC (1-60 ACN in water, HCl modifier, 15 min run) to give 3-[[4-(2,6-dimethylphenyl)-6-[(2R)-4-methyl-2-[(1-methylpyrazol-4-yl)amino]pentoxy]pyrimidin-2-yl]sulfamoyl]benzoic acid (62 mg, 45%) ESI-MS m/z calc. 578.23114, found 579.5 (M+1)⁺; Retention time: 0.48 minutes; LC method D.

### Step 3: (11R)-6-(2,6-Dimethylphenyl)-11-isobutyl-12-(1-methylpyrazol-4-yl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 97)

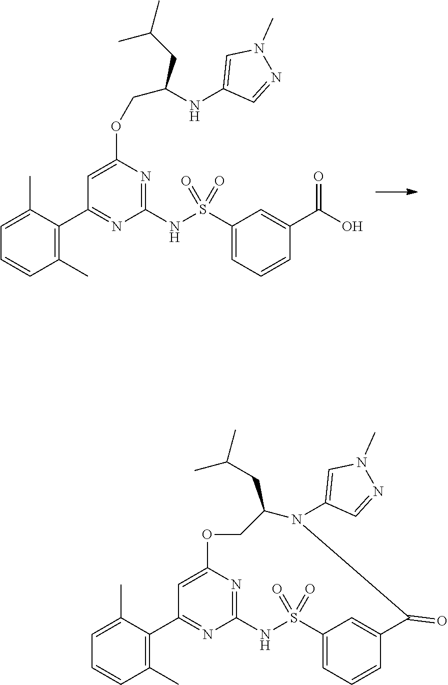 [SMILES: Cc1cccc(C)c1-c1cc(OC[C@@H](CC(C)C)Nc2cnn(C)c2)nc(NS(=O)(=O)c2cccc(C(=O)O)c2)n1 ; Cc1cccc(C)c1-c1cc2nc(n1)NS(=O)(=O)c1cccc(c1)C(=O)N(c1cnn(C)c1)[C@H](CC(C)C)CO2]

3-[[4-(2,6-dimethylphenyl)-6-[(2R)-4-methyl-2-[(1-methylpyrazol-4-yl)amino]pentoxy]pyrimidin-2-yl]sulfamoyl]benzoic acid (62 mg, 0.1071 mmol) was combined with N-methylmorpholine (approximately 43.33 mg, 47.10 μL, 0.4284 mmol) in DMF (8 mL), then cooled to 0° C. in an ice bath. 2-chloro-4,6-dimethoxy-1,3,5-triazine (approximately 28.21 mg, 0.1607 mmol) was added in one portion and the reaction mixture was allowed to warm to room temperature as the ice melted, then stirred for 65 hours at room temperature (over weekend). The reaction was concentrated, then dissolved in 1:1 DMSO/methanol, filtered, and purified by reverse phase HPLC (1-99% ACN in water, HCl modifier, 15 min run) to give (11R)-6-(2,6-dimethylphenyl)-11-isobutyl-12-(1-methylpyrazol-4-yl)-2,2-dioxo-9-oxa-2λ⁶-thia-3,5,12,19-tetrazatricyclo[12.3.1.14,8]nonadeca-1(18),4(19),5,7,14,16-hexaen-13-one (26 mg, 42%). ESI-MS m/z calc. 560.2206, found 561.4 (M+1)⁺; Retention time: 1.51 minutes; LC method A.

### Example 78: Preparation of Compound 98

### Step 1: (2R)-2-[[1-(2-Methoxyethyl)pyrazol-4-yl]amino]-4-methyl-pentan-1-ol

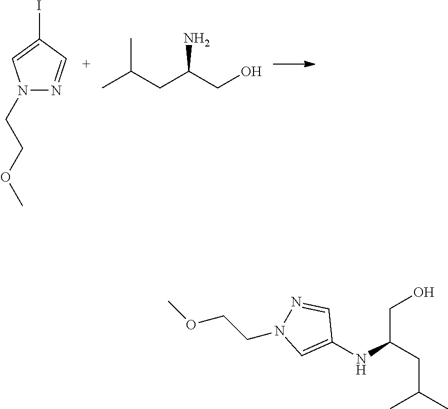

The 4-iodo-1-(2-methoxyethyl)pyrazole (approximately 215.1 mg, 0.8532 mmol) was combined with the (2R)-2-amino-4-methyl-pentan-1-ol (100 mg, 0.8533 mmol), CuI (approximately 16.25 mg, 0.08532 mmol), and NaOH (approximately 136.5 mg, 3.413 mmol) (ground with mortar and pestle) in a screw cap vial, which was then purged with nitrogen. DMSO (0.3 mL) and water (0.15 mL) were added and the reaction mixture was stirred at 90° C. for 16 hours. After cooling to room temperature, the reaction mixture was diluted with methanol and filtered. The filtrate was concentrated by rotary evaporation and the resulting residue was dissolved in 1:1 DMSO/methanol, filtered a second time and purified by reverse phase HPLC (1-50% ACN in water, HCl modifier, 15 min run) to give the indicated (2R)-2-[[1-(2-methoxyethyl)pyrazol-4-yl]amino]-4-methyl-pentan-1-ol (hydrochloride salt) (151 mg, 64%) upon drying. ESI-MS m/z calc. 241.17903, found 242.6 (M+1)⁺; Retention time: 0.28 minutes; LC method D.

### Step 2: (11R)-6-(2,6-Dimethylphenyl)-11-isobutyl-12-[1-(2-methoxyethyl)pyrazol-4-yl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 98)

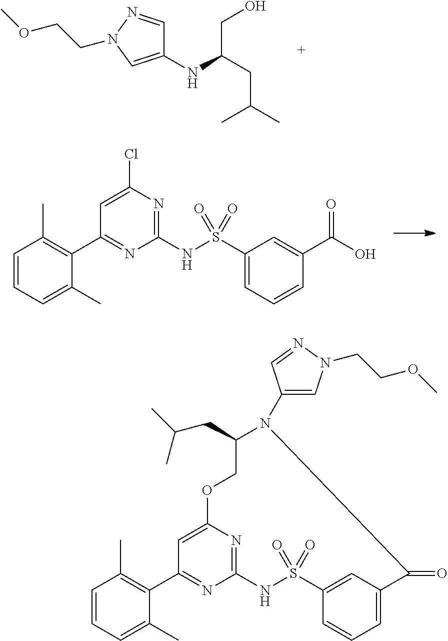

(2R)-2-[[1-(2-methoxyethyl)pyrazol-4-yl]amino]-4-methyl-pentan-1-ol (hydrochloride salt) (151 mg, 0.5436 mmol) was combined with 3-[[4-chloro-6-(2,6-dimethylphenyl)pyrimidin-2-yl]sulfamoyl]benzoic acid (approximately 174.8 mg, 0.4182 mmol) in THE (0.75 mL) and stirred until the solids had mostly dissolved. Sodium tert-butoxide (approximately 241.2 mg, 2.510 mmol) was added and the reaction briefly became slightly warm. Stirring was continued for 15 minutes with no external heating. The reaction mixture was then partitioned between 1M HCl and ethyl acetate. The layers were separated, and the aqueous was extracted an additional 4 with ethyl acetate. The combined organics were washed with brine, dried over sodium sulfate, and concentrated. The resulting crude material was dissolved in 1:1 DMSO/methanol, filtered, and purified by reverse phase HPLC (1-60 ACN in water, HCl modifier, 15 min run) to give the SNAr product, which was subsequently dissolved in DMF (8 mL) and NMM (approximately 169.2 mg, 183.9 μL, 1.673 mmol). The reaction mixture was cooled to 0° C. and CDMT (approximately 110.2 mg, 0.6274 mmol) was added. The reaction was allowed to warm to room temperature as the ice melted and it was stirred for 48 hours. The reaction mixture was quenched with several drops of water, partially concentrated, diluted with 1:1 DMSO/methanol, filtered, and purified by reverse phase HPLC (1-70% ACN in water, HCl modifier, 30 min run) to give the (11R)-6-(2,6-dimethylphenyl)-11-isobutyl-12-[1-(2-methoxyethyl)pyrazol-4-yl]-2,2-dioxo-9-oxa-2λ⁶-thia-3,5,12,19-tetrazatricyclo[12.3.1.14,8]nonadeca-1(18),4(19),5,7,14,16-hexaen-13-one (22 mg, 9%). ESI-MS m/z calc. 604.24677, found 605.5 (M+1)⁺; Retention time: 1.56 minutes; LC method A.

### Example 79: Preparation of Compound 99

### Step 1: (2R)-2-[(1-Benzylpyrazol-4-yl)amino]-4-methyl-pentan-1-ol

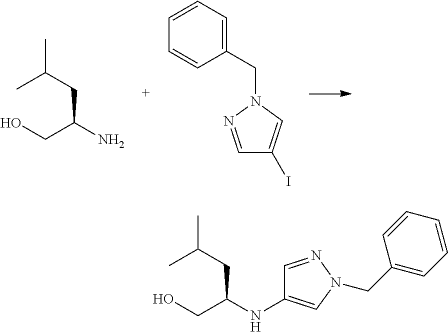

NaOH (3.4 g, 85.006 mmol) was dissolved in water (15 mL) and this solution was added to DMSO (30 mL) and nitrogen was bubbled in for 15 min. CuI (406 mg, 2.1318 mmol) was added followed by (2R)-2-amino-4-methyl-pentan-1-ol (2.5 g, 21.333 mmol) and 1-benzyl-4-iodo-pyrazole (6 g, 21.120 mmol). The reaction was stirred at 90° C. for 16 h and cooled down to room temperature. The reaction mixture was filtered on a Celite pad and rinsed with MeOH (150 mL). Volatiles were removed under vacuo. Sat aqueous ammonium chloride (50 mL) was added and the product was extracted with DCM (4×50 mL). The combined organic layers were washed with brine (50 mL), dried over sodium sulfate, filtered and concentrated under reduced pressure. The crude residue was purified by flash-chromatography on an 80 g silica gel cartridge, using a gradient of EtOAc in Heptanes (0 to 100%) and then of MeOH in EtOAc (0 to 20%). Afforded (2R)-2-[(1-benzylpyrazol-4-yl)amino]-4-methyl-pentan-1-ol (2.59 g, 42%) as a light pink solid. ESI-MS m/z calc. 273.1841, found 274.2 (M+1)⁺; Retention time: 1.3 minutes (LC method X).

### Step 2: 3-[[4-[(2R)-2-[(1-Benzylpyrazol-4-yl)amino]-4-methyl-pentoxy]-6-(2,6-dimethylphenyl)pyrimidin-2-yl]sulfamoyl]benzoic acid

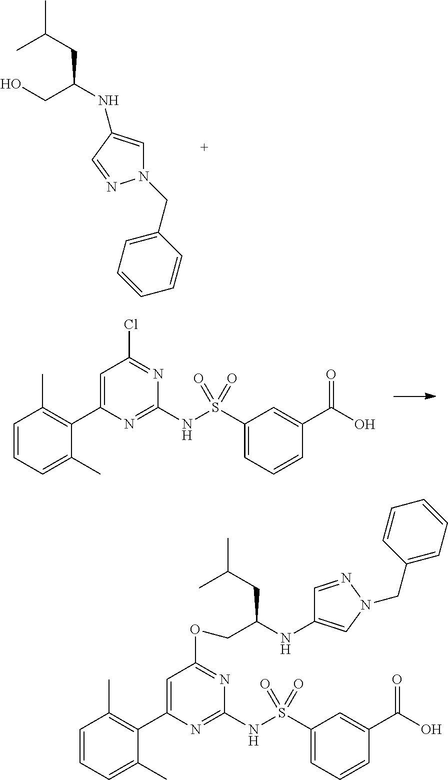

To a solution of 3-[[4-chloro-6-(2,6-dimethylphenyl)pyrimidin-2-yl]sulfamoyl]benzoic acid (hydrochloride salt) (3.3 g, 7.2635 mmol) and (2R)-2-[(1-benzylpyrazol-4-yl)amino]-4-methyl-pentan-1-ol (2 g, 7.3160 mmol) in THE (24 mL) maintained at 15° C. with a water bath was added sodium tert-butoxide (3.5 g, 36.419 mmol) and the mixture was stirred at room temperature for 0.25 h. 1N aqueous HCl (20 mL) was added and the product was extracted with EtOAc (3×20 mL). The combined organic layers were washed with brine (15 mL), dried over magnesium sulfate, filtered and concentrated under reduced pressure. Afforded crude 3-[[4-[(2R)-2-[(1-benzylpyrazol-4-yl)amino]-4-methyl-pentoxy]-6-(2,6-dimethylphenyl)pyrimidin-2-yl]sulfamoyl]benzoic acid (hydrochloride salt) (5.65 g, 105%) as a light pink solid ESI-MS m/z calc. 654.2624, found 655.2 (M+1)⁺; Retention time: 1.78 minutes (LC method X).

### Step 3: (11R)-12-(1-Benzylpyrazol-4-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18)₄(19),5,7,14,16-hexaen-13-one

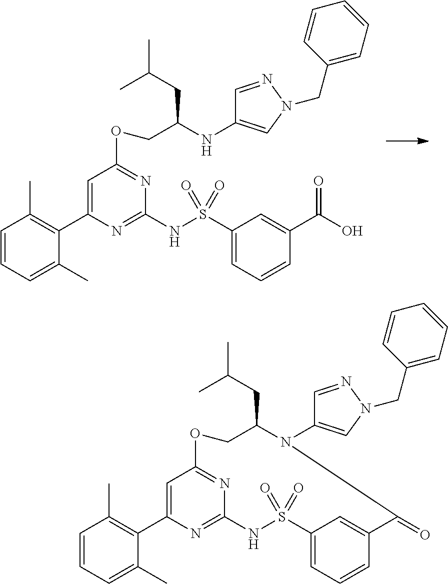

To a 0° C. solution of N-Methylmorpholine (3.4960 g, 3.8 mL, 34.564 mmol) in DMF (500 mL) was added 2-chloro-4,6-dimethoxy-1,3,5-triazine (2.2 g, 12.530 mmol) followed by 3-[[4-[(2R)-2-[(1-benzylpyrazol-4-yl)amino]-4-methyl-pentoxy]-6-(2,6-dimethylphenyl)pyrimidin-2-yl]sulfamoyl]benzoic acid (hydrochloride salt) (5.8 g, 8.0383 mmol). After 5 min the reaction went back to room temperature and was stirred for 24 h. The reaction mixture was poured onto a 1:1 v/v mix of water and brine (600 mL) and the product was extracted with MeTHF (4×150 mL). The combined organic layers were washed with a 1:1 v/v mix of water and brine (4×200 mL) and then with brine (150 mL). The resulting organic layer was dried over sodium sulfate, filtered and concentrated under reduced pressure. The crude was purified by flash chromatography using an 80 g cartridge, eluting with a gradient of EtOAc in DCM (20 to 100% in 15 CV) then with a gradient of MeOH in EtOAc (0 to 20%). Afforded (11R)-12-(1-benzylpyrazol-4-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (2.4 g, 42%) as an off-white solid. ESI-MS m/z calc. 636.2519, found 637.2 (M+1)⁺; Retention time: 4.28 minutes (LC method Y).1

### Step 4: (11R)-12-(1-Benzylpyrazol-4-yl)-6-(2,6-dimethylphenyl)-11-isobutyl-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one

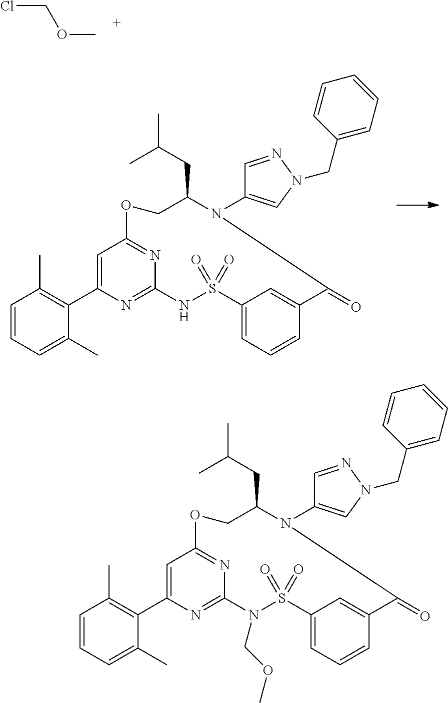

To a solution of (11R)-12-(1-benzylpyrazol-4-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (40 mg, 0.0561 mmol) in DMF (0.5 mL) at 0° C. was added potassium carbonate (15 mg, 0.1085 mmol) and chloro(methoxy)methane (5.3000 mg, 5 μL, 0.0658 mmol). The reaction was stirred at room temperature for 16 h and chloro(methoxy)methane (5.3000 mg, 5 L, 0.0658 mmol) was added. After 2 h, the reaction crude was directly loaded on a 12 g C₁₈ cartridge. Purification was run using a gradient of MeOH in acid water (0.1% v/v formic acid) of 60 to 100% in 20 CV. After evaporation afforded (11R)-12-(1-benzylpyrazol-4-yl)-6-(2,6-dimethylphenyl)-11-isobutyl-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (17 mg, 44%) as an off-white solid that contains traces of grease and EtOAc by ¹H NMR. ¹H NMR (400 MHz, DMSO-d₆) δ 8.86 (s, 1H), 8.19-8.12 (m, 1H), 8.03 (s, 1H), 7.92-7.86 (m, 1H), 7.84-7.78 (m, 1H), 7.58 (s, 1H), 7.41-7.35 (m, 2H), 7.34-7.30 (m, 1H), 7.30-7.26 (m, 2H), 7.25-7.19 (m, 1H), 7.11 (d, J=7.8 Hz, 2H), 6.67 (s, 1H), 5.71 (d, J=11.0 Hz, 1H), 5.57 (d, J=10.8 Hz, 1H), 5.43-5.36 (m, 3H), 4.06-3.91 (m, 2H), 3.03 (s, 3H), 1.97 (s, 6H), 1.60-1.45 (m, 1H), 1.37-1.25 (m, 1H), 1.11-1.01 (m, 1H), 0.68 (d, J=6.6 Hz, 3H), 0.20 (d, J=6.4 Hz, 3H). ESI-MS m/z calc. 680.2781, found 681.2 (M+1)⁺; Retention time: 2.13 minutes (LC method X).

### Step 5: (11R)-6-(2,6-dimethylphenyl)-11-isobutyl-3-(methoxymethyl)-2,2-dioxo-12-(1H-pyrazol-4-yl)-9-oxa-2λ⁶-thia-3,5,12,19-tetrazatricyclo[12.3.1.14,8]nonadeca-1(18),4(19),5,7,14,16-hexaen-13-one

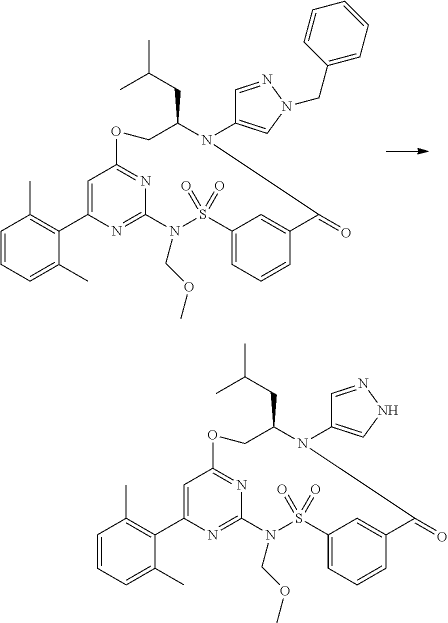

To Palladium hydroxide 20% w/w, 50% water (113 mg, 10% w/w, 0.0805 mmol) was added into a sealed tube a solution of (11R)-12-(1-benzylpyrazol-4-yl)-6-(2,6-dimethylphenyl)-11-isobutyl-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (1.1 g, 1.6141 mmol) in MeOH (10 mL). Hydrogen was bubbled in for 5 min and the sealed tube was warmed at 60° C. for 26 h. Nitrogen was bubbled into the solution for 5 min and the crude was filtered over Celite, washing with MeOH (30 mL). Volatiles were removed under reduced pressure. The crude residue was purified by flash-chromatography on a 24 g silica gel cartridge, using a gradient of AcOEt in DCM (5 to 100% in 40 CV). Afforded as first fraction starting material (11R)-12-(1-benzylpyrazol-4-yl)-6-(2,6-dimethylphenyl)-11-isobutyl-3-(methoxymethyl)-2,2-dioxo-9-oxa-2 λ6-thia-3,5,12,19-tetrazatricyclo[12.3.1.14,8]nonadeca-1(18),4(19),5,7,14,16-hexaen-13-one (698 mg, 63%) as a white solid ESI-MS m/z calc. 680.2781, found 681.2 (M+1)⁺; Retention time: 5.02 minutes (LC method S). Afforded as a second fraction (11R)-6-(2,6-dimethylphenyl)-11-isobutyl-3-(methoxymethyl)-2,2-dioxo-12-(1H-pyrazol-4-yl)-9-oxa-2λ⁶-thia-3,5,12,19-tetrazatricyclo[12.3.1.14,8]nonadeca-1(18),4(19),5,7,14,16-hexaen-13-one (220 mg, 21%) as a white solid. ¹H NMR (400 MHz, DMSO-d₆) δ 13.04 (br. s., 1H), 8.85 (s, 1H), 8.15 (d, J=8.1 Hz, 1H), 7.94 (br. s, 1H), 7.91-7.88 (m, 1H), 7.82 (t, J=7.3 Hz, 1H), 7.55 (br. s, 1H), 7.22 (t, J=7.6 Hz, 1H), 7.11 (d, J=7.6 Hz, 2H), 6.67 (s, 1H), 5.72 (d, J=11.0 Hz, 1H), 5.58 (d, J=11.0 Hz, 1H), 5.38 (dd, J=10.8, 3.9 Hz, 1H), 4.08-3.90 (m, 2H), 3.03 (s, 3H), 2.01-1.94 (m, 6H), 1.60-1.48 (m, 1H), 1.36-1.25 (m, 1H), 1.10-0.99 (m, 1H), 0.71 (d, J=6.6 Hz, 3H), 0.21 (d, J=6.4 Hz, 3H). ESI-MS m/z calc. 590.2311, found 591.2 (M+1)⁺; Retention time: 4.44 minutes (LC method S).

### Step 6: (11R)-6-(2,6-Dimethylphenyl)-11-isobutyl-2,2-dioxo-12-(1H-pyrazol-4-yl)-9-oxa-2λ⁶-thia-3,5,12,19-tetrazatricyclo[12.3.1.14,8]nonadeca-1(18),4(19),5,7,14,16-hexaen-13-one (Compound 99)

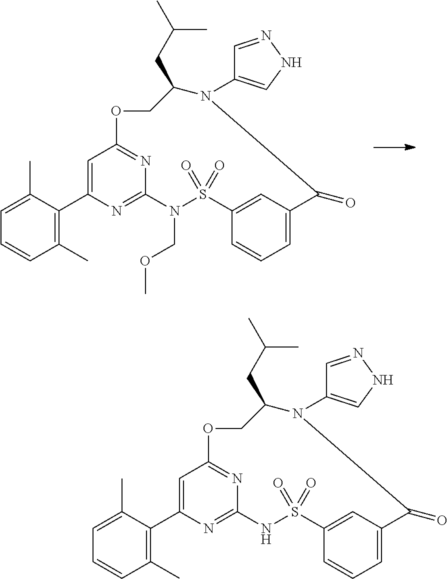

(11R)-6-(2,6-dimethylphenyl)-11-isobutyl-3-(methoxymethyl)-2,2-dioxo-12-(1H-pyrazol-4-yl)-9-oxa-2λ⁶-thia-3,5,12,19-tetrazatricyclo[12.3.1.14,8]nonadeca-1(18),4(19),5,7,14,16-hexaen-13-one (10 mg, 0.01693 mmol) was dissolved in DCM (0.15 mL) and TFA (150 μL, 1.947 mmol) was added. The reaction mixture was stirred at room temperature for 15 minutes, then was concentrated under reduced pressure. The resulting crude material was dissolved in 1:1 DMSO/methanol, filtered, and purified by prep HPLC (1-70ACN in water, HCl modifier, 15 minute run) to give upon drying (11R)-6-(2,6-dimethylphenyl)-11-isobutyl-2,2-dioxo-12-(1H-pyrazol-4-yl)-9-oxa-2λ⁶-thia-3,5,12,19-tetrazatricyclo[12.3.1.14,8]nonadeca-1(18),4(19),5,7,14,16-hexaen-13-one (6 mg, 64%). ESI-MS m/z calc. 546.2049, found 547.5 (M+1)⁺; Retention time: 1.39 minutes (LC method A).

### Example 80: Preparation of Compound 100

### Step 1: Benzyl 3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]azetidine-1-carboxylate (Compound 100)

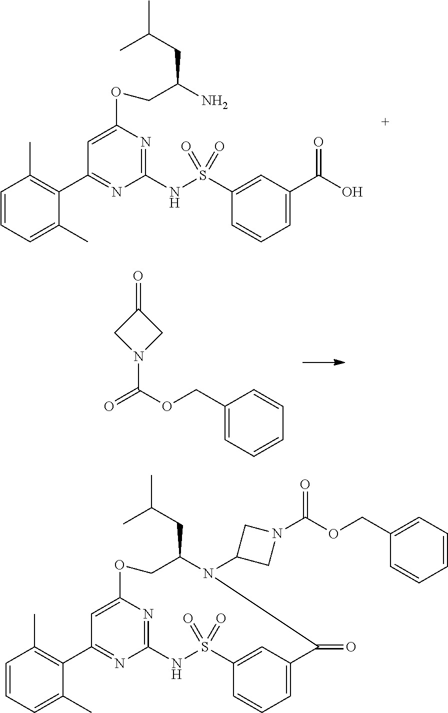 [SMILES: Cc1cccc(C)c1-c1cc(OC[C@H](N)CC(C)C)nc(NS(=O)(=O)c2cccc(C(=O)O)c2)n1 ; O=C1CN(C(=O)OCc2ccccc2)C1 ; Cc1cccc(C)c1-c1cc2nc(n1)NS(=O)(=O)c1cccc(c1)C(=O)N(C1CN(C(=O)OCc3ccccc3)C1)[C@H](CC(C)C)CO2]

3-[[4-[(2R)-2-amino-4-methyl-pentoxy]-6-(2,6-dimethylphenyl)pyrimidin-2-yl]sulfamoyl]benzoic acid (hydrochloride salt) (300 mg, 0.5607 mmol) and benzyl 3-oxoazetidine-1-carboxylate (230 mg, 1.121 mmol) were combined in dichloromethane (1.25 mL) and sodium triacetoxyborohydride (300 mg, 1.415 mmol) was added. The reaction mixture became homogenous after several minutes and after one hour at room temperature, additional sodium triacetoxyborohydride (300 mg, 1.415 mmol), followed by additional benzyl 3-oxoazetidine-1-carboxylate (230 mg, 1.121 mmol) were added. After a further hour, the reaction was poured into a separatory funnel containing 0.5M HCl and ethyl acetate (50 mL each). The layers were separated, and the aqueous was extracted an additional 2×25 mL ethyl acetate, and the combined organics were washed with brine, dried over sodium sulfate and concentrated. The resulting material was used in the next step without purification, 3-[[4-[(2R)-2-[(1-benzyloxycarbonylazetidin-3-yl)amino]-4-methyl-pentoxy]-6-(2,6-dimethylphenyl)pyrimidin-2-yl]sulfamoyl]benzoic acid (hydrochloride salt) ESI-MS m/z calc. 687.27264, found 688.4 (M+1)⁺; Retention time: 0.54 minutes (LC method D). The crude product was combined with HATU (425 mg, 1.118 mmol) in DMF (60 mL) and DIPEA (500 μL, 2.871 mmol) was added. The reaction was stirred for an additional 20 hours at room temperature, then was partially concentrated under reduced pressure. The reaction mixture was partitioned between ethyl acetate and 1M HCl and the layers were separated. The aqueous was extracted an additional 2×with ethyl acetate, and the combined organics were washed with 1M HCl, brine, and dried over sodium sulfate. After concentrating, the crude material was purified by chromatography on silica gel eluting with 0-100% ethyl acetate in dichloromethane (to give a yellowish solid benzyl 3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]azetidine-1-carboxylate (108 mg, 29%) ESI-MS m/z calc. 669.2621, found 670.3 (M+1)⁺; Retention time: 0.74 minutes (LC method D). 10 mg of the above product were subjected to additional purification by reverse phase HPLC (1-99% ACN in water, no modifier) to give as a white solid, benzyl 3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]azetidine-1-carboxylate (3.8 mg, 1%). ESI-MS m/z calc. 669.2621, found 670.3 (M+1)⁺; Retention time: 1.87 minutes (LC method A).

### Example 81: Preparation of Compound 101

### Step 1: (11R)-12-(Azetidin-3-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one

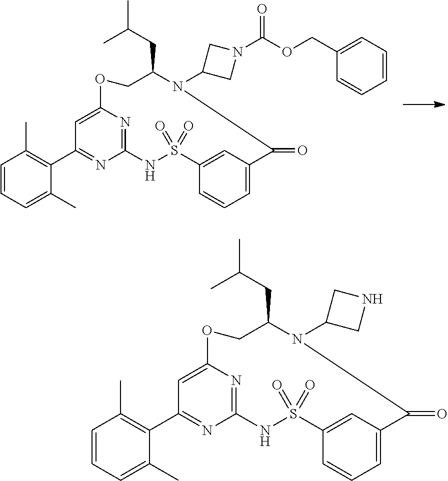

Benzyl 3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]azetidine-1-carboxylate (102 mg, 0.1523 mmol) was dissolved in methanol (1.5 mL), and dihydroxypalladium (15 mg, 0.02136 mmol) was added. The reaction vessel was purged with nitrogen then hydrogen gas from a balloon was bubbled through the reaction mixture for 30 minutes. The reaction was stirred at room temperature for an additional 90 minutes with the hydrogen balloon in place. (The product had extremely poor solubility in methanol and could be observed crashing out of solution during the course of the reaction. The reaction mixture was purged with nitrogen and filtered eluting with 150 mL methanol. The filtrate was concentrated to give a gray solid (11R)-12-(azetidin-3-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (60 mg, 74%) ESI-MS m/z calc. 535.22534, found 536.3 (M+1)⁺; Retention time: 0.46 minutes (LC method D).

### Step 2: (11R)-6-(2,6-Dimethylphenyl)-11-isobutyl-2,2-dioxo-12-(1-spiro[2.3]hexan-5-ylazetidin-3-yl)-9-oxa-2λ⁶-thia-3,5,12,19-tetrazatricyclo[12.3.1.14,8]nonadeca-1(18),4(19),5,7,14,16-hexaen-13-one (Compound 101)

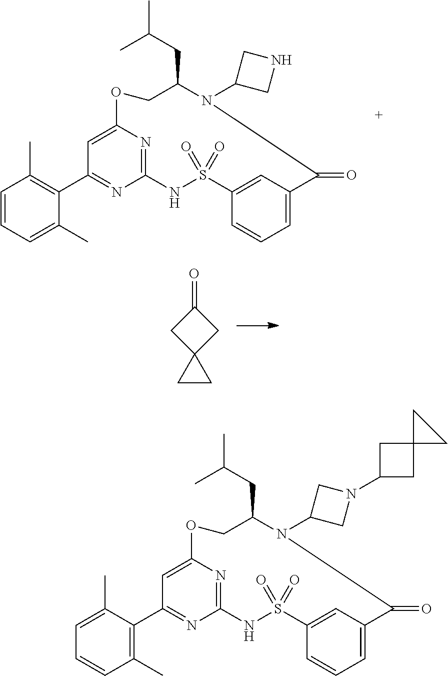 [SMILES: Cc1cccc(C)c1-c1cc2nc(n1)NS(=O)(=O)c1cccc(c1)C(=O)N(C1CNC1)[C@H](CC(C)C)CO2 ; O=C1CC2(CC2)C1 ; Cc1cccc(C)c1-c1cc2nc(n1)NS(=O)(=O)c1cccc(c1)C(=O)N(C1CN(C3CC4(CC4)C3)C1)[C@H](CC(C)C)CO2]

(11R)-12-(azetidin-3-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (11 mg, 0.02054 mmol) was combined with the spiro[2.3]hexan-5-one (approximately 5.923 mg, 0.06162 mmol) and acetic acid (approximately 2.467 mg, 2.336 μL, 0.04108 mmol) in DCM (0.35 mL). After stirring 10 minutes at room temperature, sodium triacetoxyborohydride (approximately 13.06 mg, 0.06162 mmol) was added, followed by a second addition of sodium triacetoxyborohydride (approximately 13.06 mg, 0.06162 mmol) after 45 minutes. After 90 minutes reaction time the reaction mixture was concentrated and dissolved in methanol then filtered and purified by reverse phase HPLC (1-99% ACN in water, HCl modifier). Fractions containing product were diluted with an equal volume of acetonitrile and concentrated by rotary evaporation to give (11R)-6-(2,6-dimethylphenyl)-11-isobutyl-2,2-dioxo-12-(1-spiro[2.3]hexan-5-ylazetidin-3-yl)-9-oxa-2λ⁶-thia-3,5,12,19-tetrazatricyclo[12.3.1.14,8]nonadeca-1(18),4(19),5,7,14,16-hexaen-13-one (hydrochloride salt) (1.6 mg, 12%). ESI-MS m/z calc. 615.2879, found 616.4 (M+1)⁺; Retention time: 1.31 minutes; LC method A.

### Example 82: Preparation of Compound 102

### Step 1: (11R)-12-(2-Azaspiro[3.3]heptan-6-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one

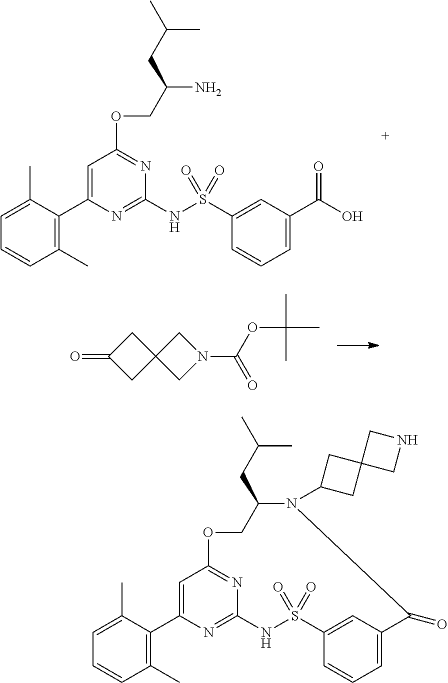

3-[[4-[(2R)-2-amino-4-methyl-pentoxy]-6-(2,6-dimethylphenyl)pyrimidin-2-yl]sulfamoyl]benzoic acid (hydrochloride salt) (150 mg, 0.2803 mmol) was combined with tert-butyl 6-oxo-2-azaspiro[3.3]heptane-2-carboxylate (90 mg, 0.4260 mmol) in DCM (0.5 mL) and after five minutes stirring at room temperature, sodium triacetoxyborohydride (180 mg, 0.8493 mmol) was added. After one hour, an additional portion of sodium triacetoxyborohydride (180 mg, 0.8493 mmol) was added, and two hours later a final portion of sodium triacetoxyborohydride (90 mg, 0.4246 mmol) was added, followed by a final hour stirring at room temperature. The reaction mixture was then partitioned between 0.5M HCl and ethyl acetate. The layers were separated, and the aqueous layer was extracted an additional three times with ethyl acetate. The combined organics were washed with brine, dried over sodium sulfate and concentrated. The resulting material was dissolved in DMF (10 mL) and added over two minutes to a stirring solution of HATU (160 mg, 0.4208 mmol) and DIPEA (250 μL, 1.435 mmol) in DMF (10 mL). After three hours at room temperature, the reaction mixture was partitioned between 1M HCl and ethyl acetate. The layers were separated, and the aqueous layer was extracted an additional two times with ethyl acetate. The combined organics were washed with brine and dried over sodium sulfate then concentrated. The resulting crude material was purified by flash chromatography on silica gel, eluting with a 1-100% gradient of ethyl acetate in hexanes. Fractions containing product were combined and concentrated to give a foaming solid (109 mg). The product was dissolved in DCM (10 mL) and TFA (325 μL, 4.218 mmol) was added. The reaction mixture was stirred at room temperature for 15 minutes, then was diluted with dichloromethane and concentrated. 1,2-dichloroethane was added and the reaction mixture was concentrated a second time to give (11R)-12-(2-azaspiro[3.3]heptan-6-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (trifluoroacetate salt) (120 mg, 62%) ESI-MS m/z calc. 575.25665, found 576.6 (M+1)⁺; Retention time: 0.52 minutes (LC method D).

### Step 2: Isopropyl 6-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-2-azaspiro[3.3]heptane-2-carboxylate (Compound 102)

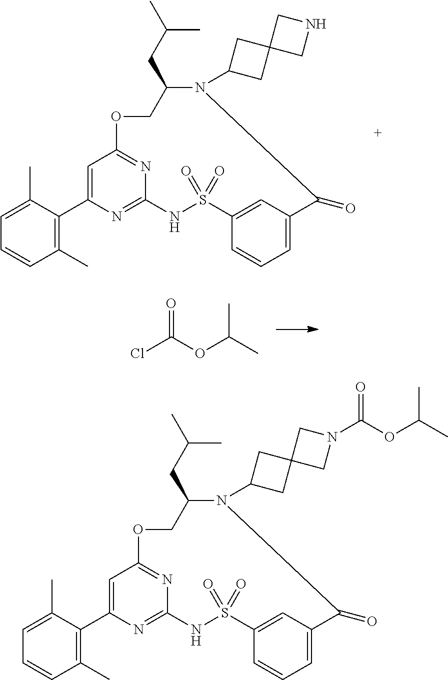

The (11R)-12-(2-azaspiro[3.3]heptan-6-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (trifluoroacetate salt) (20 mg, 0.02900 mmol) macrocycle was combined in DCM (0.5 mL) with DIPEA (approximately 22.49 mg, 30.31 μL, 0.1740 mmol) and isopropyl chloroformate (approximately 29.00 μL of 2 M, 0.05800 mmol) then stirred at room temperature for 10 minutes. The reaction mixture was then quenched with 0.5M HCl, partially concentrated, diluted with methanol and DMSO (1:1), filtered, and purified by reverse phase HPLC (1-99% ACN in water, HCl modifier, 15 min run) to give the corresponding isopropyl 6-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-2-azaspiro[3.3]heptane-2-carboxylate (1.7 mg, 9%) as a white powder. ESI-MS m/z calc. 661.2934, found 662.5 (M+1)⁺; Retention time: 1.91 minutes; LC method A.

### Example 83: Preparation of Compound 103

### Step 1: (11R)-6-(2,6-Dimethylphenyl)-11-isobutyl-12-(2-isopropyl-2-azaspiro[3.3]heptan-6-yl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 103)

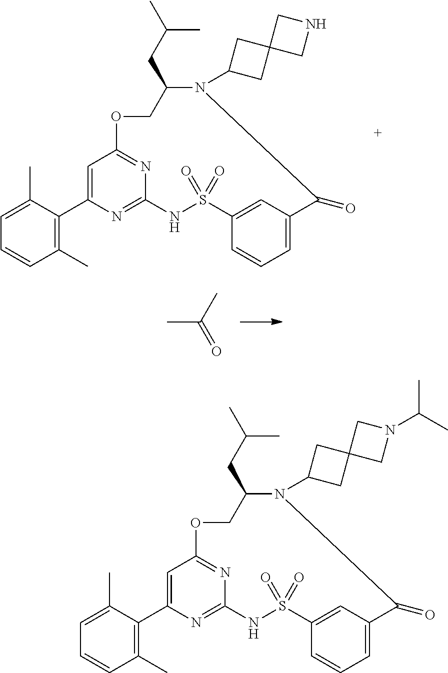

(11R)-12-(2-azaspiro[3.3]heptan-6-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (trifluoroacetate salt) (32 mg, 0.04639 mmol) was combined with acetone (25 μL, 0.3405 mmol) in DCM (0.4 mL) and stirred at room temperature for 5 minutes. Sodium triacetoxyborohydride (100 mg, 0.4718 mmol) was added and the reaction mixture was stirred for an additional 10 minutes at room temperature. The reaction mixture was then diluted with methanol and acetic acid, filtered, and purified by reverse phase HPLC (1-99% ACN in water, HCl modifier, 15 min run.). Fractions containing product were combined and evaporated to dryness with a bath temperature of 35° C. (intermediates had shown some HCl sensitivity) to give (11R)-6-(2,6-dimethylphenyl)-11-isobutyl-12-(2-isopropyl-2-azaspiro[3.3]heptan-6-yl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (4.1 mg, 13%) ESI-MS m/z calc. 617.3036, found 618.8 (M+1)⁺; Retention time: 1.26 minutes (LC method A).

### Example 84: Preparation of Compound 104

### Step 1: (11R)-6-(2,6-Dimethylphenyl)-11-isobutyl-12-[2-(2-methoxyethyl)-2-azaspiro[3.3]heptan-6-yl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 104)

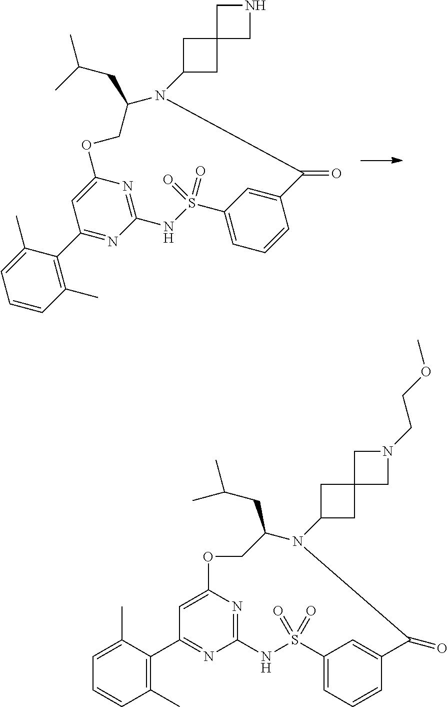 [SMILES: Cc1cccc(C)c1-c1cc2nc(n1)NS(=O)(=O)c1cccc(c1)C(=O)N(C1CC3(CNC3)C1)[C@H](CC(C)C)CO2 ; COCCN1CC2(CC(N3C(=O)c4cccc(c4)S(=O)(=O)Nc4nc(cc(-c5c(C)cccc5C)n4)OC[C@H]3CC(C)C)C2)C1]

(11R)-12-(2-azaspiro[3.3]heptan-6-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (60 mg, 0.1042 mmol) was combined with 1-bromo-2-methoxy-ethane (20 μL, 0.2128 mmol) in acetonitrile (500 μL) and triethylamine (75 μL, 0.5381 mmol) was added. The reaction mixture was heated to 60° C. for 6 h. The reaction mixture was then cooled to room temperature and filtered then purified by reverse phase HPLC (1-99% ACN in water, HCl modifier, 30 min run). The resulting fractions were dried to give as a white solid, (11R)-6-(2,6-dimethylphenyl)-11-isobutyl-12-[2-(2-methoxyethyl)-2-azaspiro[3.3]heptan-6-yl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (16.8 mg, 23%). ¹H NMR (400 MHz, DMSO-d₆) δ 10.2 (bs, 1H), 8.41 (s, 1H), 7.93 (d, J=15.3 Hz, 1H), 7.67 (s, 2H), 7.26 (s, 1H), 7.12 (s, 2H), 6.52 (s, 1H), 6.37 (s, 1H), 5.16-5.05 (m, 1H), 4.39-4.01 (m, 5H), 4.01-3.93 (m, 1H), 3.71 (s, 1H), 3.50 (d, J=4.9 Hz, 2H), 3.30 (s, 3H), 3.05 (s, 2H), 1.93 (d, J=17.1 Hz, 7H), 1.55 (t, J=12.8 Hz, 2H), 1.24 (s, 2H), 1.13 (t, J=12.1 Hz, 1H), 0.73 (d, J=6.5 Hz, 3H), 0.19 (d, J=6.2 Hz, 3H). ESI-MS m/z calc. 633.29846, found 634.6 (M+1)⁺; Retention time: 1.29 minutes (LC method A).

### Example 85: Preparation of Compound 105

### Step 1: tert-butyl N-[[3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]methyl]carbamate, diastereomer 1, and tert-butyl N-[[3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]methyl]carbamate, diastereomer 2

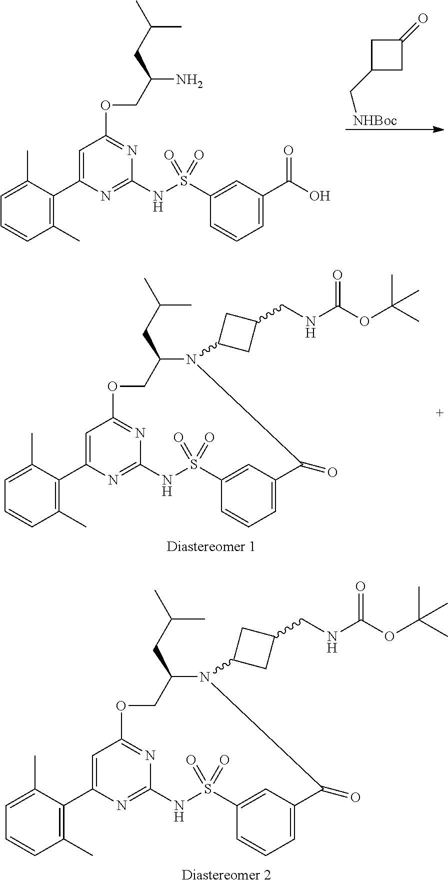 [SMILES: Cc1cccc(C)c1-c1cc(OC[C@H](N)CC(C)C)nc(NS(=O)(=O)c2cccc(C(=O)O)c2)n1 ; CC(C)(C)OC(=O)NCC1CC(=O)C1 ; Cc1cccc(C)c1-c1cc2nc(n1)NS(=O)(=O)c1cccc(c1)C(=O)N(C1CC(CNC(=O)OC(C)(C)C)C1)[C@H](CC(C)C)CO2 ; Cc1cccc(C)c1-c1cc2nc(n1)NS(=O)(=O)c1cccc(c1)C(=O)N(C1CC(CNC(=O)OC(C)(C)C)C1)[C@H](CC(C)C)CO2]

3-[[4-[(2R)-2-amino-4-methyl-pentoxy]-6-(2,6-dimethylphenyl)pyrimidin-2-yl]sulfamoyl]benzoic acid (hydrochloride salt) (250 mg, 0.4672 mmol) was combined with tert-butyl N-[(3-oxocyclobutyl)methyl]carbamate (120 mg, 0.6023 mmol) in DCM (800 μL), and stirred for 15 minutes at room temperature. Sodium triacetoxyborohydride (300 mg, 1.415 mmol) was added and the reaction mixture was stirred for an additional hour. tert-butyl N-[(3-oxocyclobutyl)methyl]carbamate (50 mg, 0.2509 mmol) was added followed by additional sodium triacetoxyborohydride (300 mg, 1.415 mmol) and the reaction was stirred at room temperature for an additional 4 hours. The reaction mixture was then partitioned between 1M HCl and ethyl acetate. The layers were separated and the aqueous was extracted an additional 3×with ethyl acetate. The combined organics were washed with brine, dried over sodium sulfate, and concentrated. This material dissolved in DMF (5 mL) was added dropwise to a stirring solution of HATU (355 mg, 0.9336 mmol) and DIPEA (400 μL, 2.296 mmol) in DMF (15 mL) over 2 minutes. The reaction mixture was stirred at room temperature for 7 hours then was partitioned between 1M HCl and ethyl acetate. The layers were separated and the aqueous was extracted an additional 3×with ethyl acetate. The combined organics were washed with brine, dried over sodium sulfate and concentrated. The resulting crude material was purified by reverse phase chromatography using a gradient of (1-99% MeOH in water, HCl modifier (shallow in the middle, 30 min run) to give separately two stereoisomers of the cyclobutane: diastereomer 1, peak 1 tert-butyl N-[[3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]methyl]carbamate (45 mg, 15%) ESI-MS m/z calc. 663.3091, found 664.6 (M+1)⁺; Retention time: 0.76 minutes (LC method D), and diastereomer 2, peak 2 tert-butyl N-[[3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]methyl]carbamate (60 mg, 19%) ESI-MS m/z calc. 663.3091, found 664.6 (M+1)⁺; Retention time: 0.77 minutes (LC method D).

### Step 2: (11R)-12-[3-(Aminomethyl)cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1

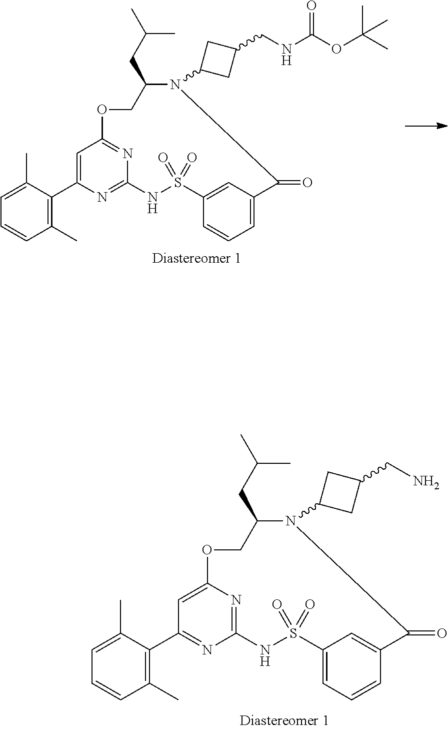 [SMILES: Cc1cccc(C)c1-c1cc2nc(n1)NS(=O)(=O)c1cccc(c1)C(=O)N(C1CC(CNC(=O)OC(C)(C)C)C1)[C@H](CC(C)C)CO2 ; Cc1cccc(C)c1-c1cc2nc(n1)NS(=O)(=O)c1cccc(c1)C(=O)N(C1CC(CN)C1)[C@H](CC(C)C)CO2]

tert-butyl N-[[3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]methyl]carbamate, diastereomer 1 (45 mg, 0.06779 mmol) was combined with HCl (approximately 254.2 μL of 4 M, 1.017 mmol) in DCM (0.3 mL), and stirred at room temperature for 30 minutes. The reaction mixture was then evaporated, hexanes were added, and the reaction was evaporated a second time to give a white powder (11R)-12-[3-(aminomethyl)cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (44 mg, 108%) ESI-MS m/z calc. 563.25665, found 564.6 (M+1)⁺; Retention time: 0.51 minutes; LC method D.

### Step 3: (11R)-12-[3-[(Dimethylamino)methyl]cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 105)

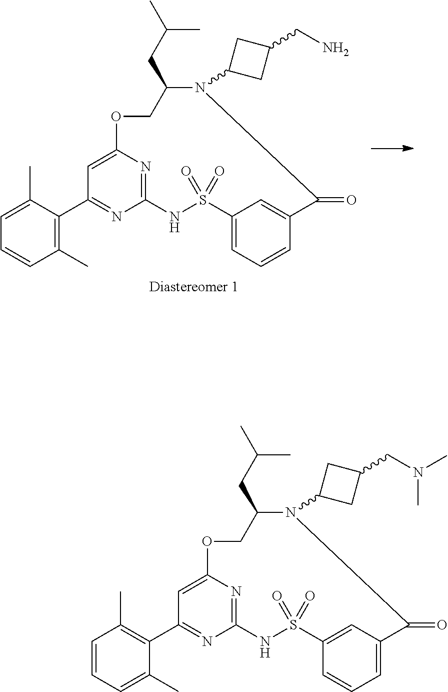 [SMILES: Cc1cccc(C)c1-c1cc2nc(n1)NS(=O)(=O)c1cccc(c1)C(=O)N(C1CC(CN)C1)[C@H](CC(C)C)CO2 ; Cc1cccc(C)c1-c1cc2nc(n1)NS(=O)(=O)c1cccc(c1)C(=O)N(C1CC(CN(C)C)C1)[C@H](CC(C)C)CO2]

(11R)-12-[3-(aminomethyl)cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) diastereomer 1 (15 mg, 0.02499 mmol) was combined with formaldehyde (0.25 mL, 9.075 mmol) (aqueous) and formic acid (0.2 mL) in a screwcap vial with an unpierced septum. The reaction mixture was heated to 95° C. for 18 hours. The reaction mixture was then cooled to room temperature, diluted with methanol, and purified by reverse phase HPLC (1-70% ACN in water, HCl modifier) to give as a white powder, (11R)-12-[3-[(dimethylamino)methyl]cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (9 mg, 57%). ESI-MS m/z calc. 591.2879, found 592.7 (M+1)⁺; Retention time: 1.22 minutes; LC method A.

### Example 86: Preparation of Compound 106

### Step 1: (11R)-12-[3-(Aminomethyl)cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 2

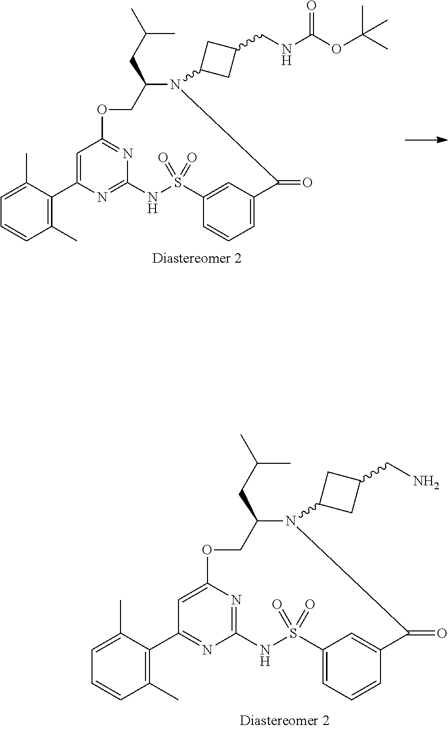 [SMILES: Cc1cccc(C)c1-c1cc2nc(n1)NS(=O)(=O)c1cccc(c1)C(=O)N(C1CC(CNC(=O)OC(C)(C)C)C1)[C@H](CC(C)C)CO2 ; Cc1cccc(C)c1-c1cc2nc(n1)NS(=O)(=O)c1cccc(c1)C(=O)N(C1CC(CN)C1)[C@H](CC(C)C)CO2]

tert-butyl N-[[3-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]methyl]carbamate, diastereomer 2 (45 mg, 0.06779 mmol) was combined with HCl (approximately 254.2 μL of 4 M, 1.017 mmol) in DCM (0.3 mL), and stirred at room temperature for 30 minutes. The reaction mixture was then evaporated, hexanes were added, and the reaction was evaporated a second time to give a white powder (11R)-12-[3-(aminomethyl)cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (57 mg, 105%) ESI-MS m/z calc. 563.25665, found 564.7 (M+1)⁺; Retention time: 0.52 minutes; LC method D.

### Step 2: (11R)-12-[3-[(Dimethylamino)methyl]cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 106)

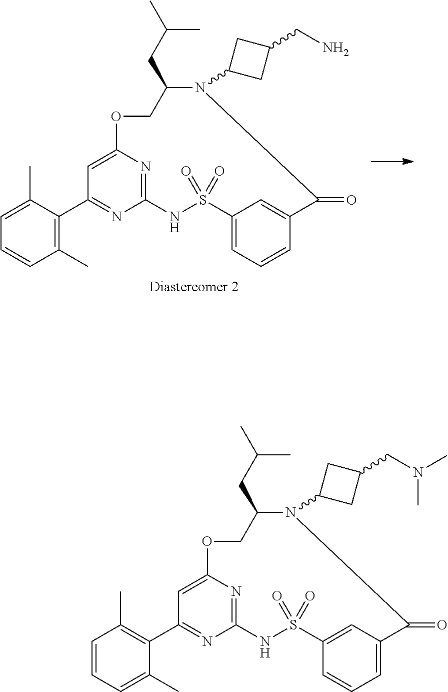 [SMILES: Cc1cccc(C)c1-c1cc2nc(n1)NS(=O)(=O)c1cccc(c1)C(=O)N(C1CC(CN)C1)[C@H](CC(C)C)CO2 ; Cc1cccc(C)c1-c1cc2nc(n1)NS(=O)(=O)c1cccc(c1)C(=O)N(C1CC(CN(C)C)C1)[C@H](CC(C)C)CO2]

(11R)-12-[3-(aminomethyl)cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) diastereomer 1 (15 mg, 0.02499 mmol) was combined with formaldehyde (0.25 mL, 9.075 mmol) (aqueous) and formic acid (0.2 mL) in a screwcap vial with an unpierced septum. The reaction mixture was heated to 95° C. for 18 hours. The reaction mixture was then cooled to room temperature, diluted with methanol, and purified by reverse phase HPLC (1-70% ACN in water, HCl modifier) to give as a white powder, (11R)-12-[3-[(dimethylamino)methyl]cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (7.8 mg, 49%). ESI-MS m/z calc. 591.2879, found 592.9 (M+1)⁺; Retention time: 1.24 minutes; LC method A.

### Example 87: Preparation of Compound 107

### Step 1: Propan-2-yl N-({3-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(18),4(19),5,7,14,16-hexaen-12-yl]cyclobutyl}methyl)carbamate (Compound 107)

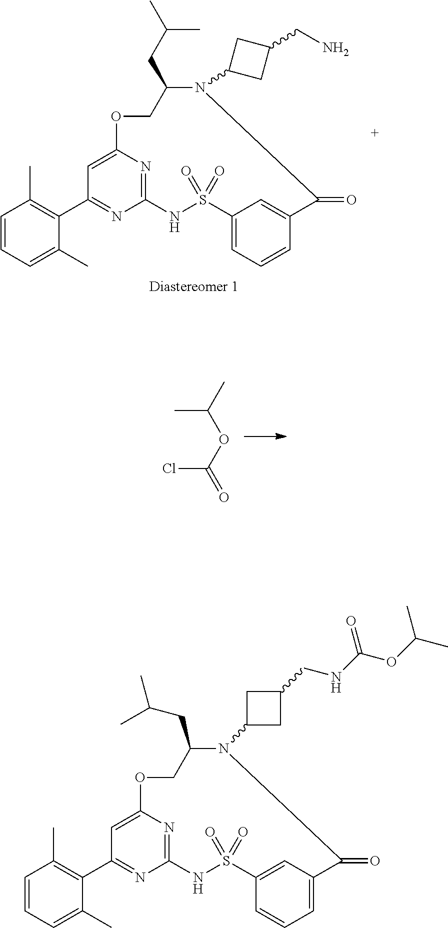

To a solution of the (11R)-12-[3-(aminomethyl)cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) diastereomer 1 (10 mg, 0.01666 mmol) in DCM (0.5 mL) was added isopropyl chloroformate (approximately 16.66 μL of 2 M in toluene, 0.03332 mmol) followed by DIEA (approximately 10.77 mg, 0.08330 mmol). The reaction mixture was stirred for 30 minutes at room temperature, then was quenched with several drops of 1M HCl and partially concentrated. The resulting crude material was dissolved in 1:1 DMSO/methanol, filtered and purified by reverse phase HPLC (1-99% ACN in water, HCl modifier, 15 min run) to give upon drying -2-yl N-({3-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(18),4(19),5,7,14,16-hexaen-12-yl]cyclobutyl}methyl)carbamate (6.5 mg, 59%). ESI-MS m z calc. 649.2934, found 650.6 (M+1)⁺; Retention time: 1.85 minutes; LC method A.

### Example 88: Preparation of Compound 108

### Step 1: Propan-2-yl N-({3-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(18),4(19),5,7,14,16-hexaen-12-yl]cyclobutyl}methyl)carbamate (Compound 108)

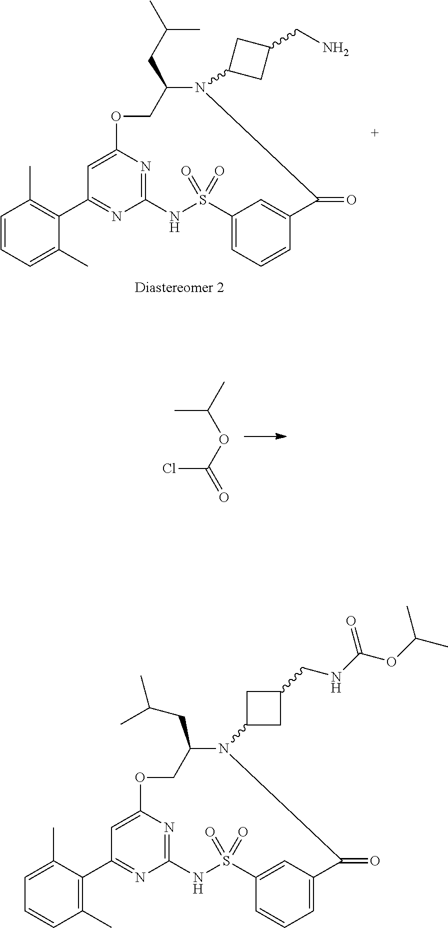

To a solution of the (11R)-12-[3-(aminomethyl)cyclobutyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) diastereomer 2 (10 mg, 0.01666 mmol) in DCM (0.5 mL) was added isopropyl chloroformate (approximately 16.66 μL of 2 M, 0.03332 mmol) followed by DIEA (approximately 10.77 mg, 0.08330 mmol). The reaction mixture was stirred for 30 minutes at room temperature, then was quenched with several drops of 1M HCl and partially concentrated. The resulting crude material was dissolved in 1:1 DMSO/methanol, filtered and purified by reverse phase HPLC (1-99% ACN in water, HCl modifier, 15 min run) to give upon drying -2-yl N-({3-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(18),4(19),5,7,14,16-hexaen-12-yl]cyclobutyl}methyl)carbamate (6.3 mg, 59%). ESI-MS m z calc. 649.2934, found 650.8 (M+1)⁺; Retention time: 1.90 minutes; LC method A.

### Example 89: Preparation of (11S)-6-(2,6-Dimethylphenyl)-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (Compound 474)

### Step 1: (2S)-4-Methyl-2-(spiro[2.3]hexan-5-ylamino)pentan-1-ol

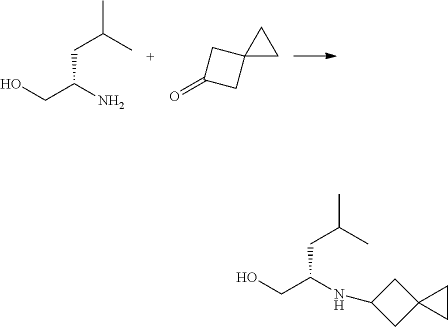

To a stirring solution of (2S)-2-amino-4-methyl-pentan-1-ol (1.93 g, 16.469 mmol) and spiro[2.3]hexan-5-one (1.507 g, 15.677 mmol) in anhydrous 1,2-dichloroethane (22 mL) at room temperature under nitrogen was portionwise added sodium triacetoxyborohydride (4.99 g, 23.544 mmol). After the addition was complete, the reaction mixture was stirred at this temperature for 20 hours. The reaction mixture was diluted with DCM (25 mL) and 1 M aqueous HCl (120 mL) was slowly added (pH˜1). The reaction mixture was stirred for 15 minutes. Two layers were separated, and the organic layer was discarded. The aqueous layer was basified with 2 M aqueous NaOH (80 mL) to pH˜12, and the product was extracted with ethyl acetate (3×100 mL). The combined organic layers were washed with brine (40 mL), dried over anhydrous sodium sulfate and concentrated to afford (2S)-4-methyl-2-(spiro[2.3]hexan-5-ylamino)pentan-1-ol (2.65 g, 81%) as pale-yellow oil. The product was carried to the next step without further purification. ESI-MS m/z calc. 197.178, found 198.5 (M+1)⁺; Retention time: 2.48 minutes (LC method S). ¹H NMR (250 MHz, DMSO-d₆) δ 4.41 (s, 1H), 3.55-3.40 (m, 1H), 3.29-3.12 (m, 2H), 2.49-2.37 (m, 1H), 2.16-1.99 (m, 2H), 1.99-1.83 (m, 2H), 1.78-1.52 (m, 2H), 1.13 (t, J=6.8 Hz, 2H), 0.94-0.78 (m, 6H), 0.49-0.22 (m, 4H).

### Step 2: 3-[[4-(2,6-Dimethylphenyl)-6-[(2S)-4-methyl-2-(spiro[2.3]hexan-5-ylamino)pentoxy]pyrimidin-2-yl]sulfamoyl]benzoic acid

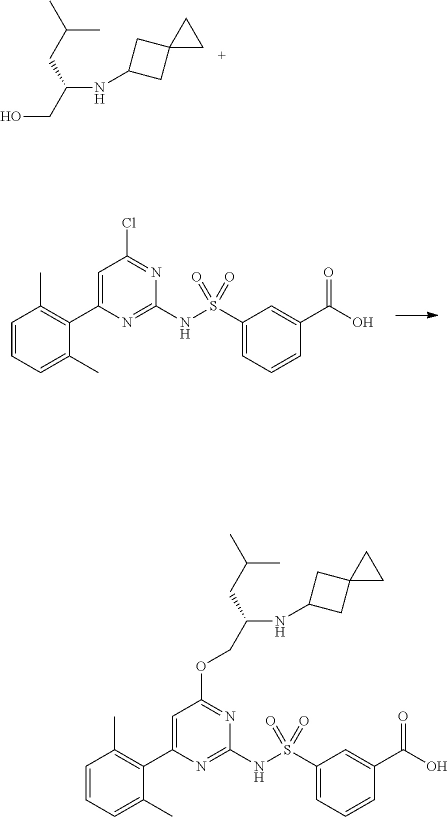 [SMILES: CC(C)C[C@@H](CO)NC1CC2(CC2)C1 ; Cc1cccc(C)c1-c1cc(Cl)nc(NS(=O)(=O)c2cccc(C(=O)O)c2)n1 ; Cc1cccc(C)c1-c1cc(OC[C@H](CC(C)C)NC2CC3(CC3)C2)nc(NS(=O)(=O)c2cccc(C(=O)O)c2)n1]

To a stirring solution of (2S)-4-methyl-2-(spiro[2.3]hexan-5-ylamino)pentan-1-ol (2.65 g, 13.430 mmol) and 3-[[4-chloro-6-(2,6-dimethylphenyl)pyrimidin-2-yl]sulfamoyl]benzoic acid (5.65 g, 13.521 mmol) in anhydrous THE (40 mL) at room temperature under nitrogen was portionwise added sodium tert-butoxide (5.24 g, 54.525 mmol). After the addition was complete, the reaction mixture was stirred at this temperature for 2 hours. The reaction was slowly acidified with 1 M aqueous HCl (80 mL) to pH˜1, and the reaction mixture was stirred for 15 minutes. The reaction mixture was poured into hexanes (250 mL) and stirred vigorously for 10 minutes. Precipitated product was collected by filtration, washed with hexanes (2×50 mL) and dried under vacuum to afford 3-[[4-(2,6-dimethylphenyl)-6-[(2S)-4-methyl-2-(spiro[2.3]hexan-5-ylamino)pentoxy]pyrimidin-2-yl]sulfamoyl]benzoic acid (hydrochloride salt) (8.258 g, 85%) as white solid. The product was carried to the next step without further purification. ESI-MS m/z calc. 578.2563, found 579.6 (M+1)⁺; Retention time: 4.2 minutes (LC method S).

### Step 3: (11S)-6-(2,6-Dimethylphenyl)-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (Compound 474)

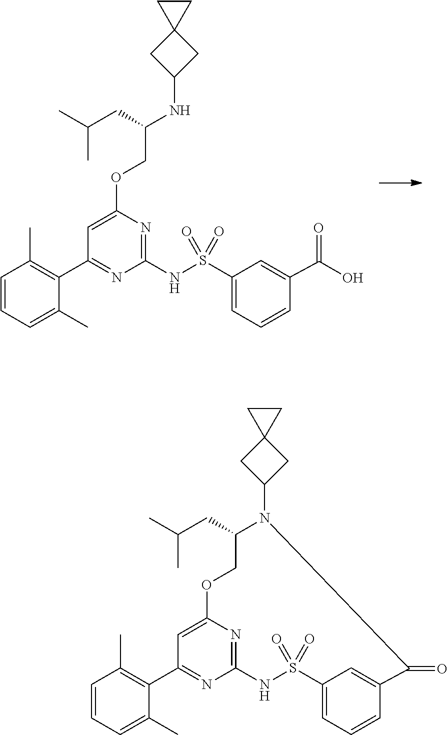 [SMILES: Cc1cccc(C)c1-c1cc(OC[C@H](CC(C)C)NC2CC3(CC3)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CC3)C1)[C@@H](CC(C)C)CO2]

A solution of 3-[[4-(2,6-dimethylphenyl)-6-[(2S)-4-methyl-2-(spiro[2.3]hexan-5-ylamino)pentoxy]pyrimidin-2-yl]sulfamoyl]benzoic acid (hydrochloride salt) (51.3 mg, 0.08339 mmol), [[(E)-(1-cyano-2-ethoxy-2-oxo-ethylidene)amino]oxy-tetrahydropyran-4-yl-methylene]-dimethyl-ammonium (Phosphorus Hexafluoride Ion) (57 mg, 0.1334 mmol), and triethylamine (47.8 μL, 0.3429 mmol) in DMF (4.275 mL) was stirred overnight. The reaction was concentrated, filtered and purified using a reverse phase HPLC-MS method using a Luna C₁₈ (2) column (75×30 mm, 5 m particle size) sold by Phenomenex (pn: 00C-4252-U0-AX), and a dual gradient run from 1-99% mobile phase B over 15.0 minutes (mobile phase A=water (5 mM HCl), mobile phase B=acetonitrile, flow rate=50 mL/min, injection volume=950 μL and column temperature=25° C.) to give (11S)-6-(2,6-dimethylphenyl)-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (36.9 mg, 79%) as a white solid. ¹H NMR (400 MHz, DMSO-d₆) δ 13.06 (s, 1H), 8.40 (s, 1H), 7.91 (s, 1H), 7.68 (s, 2H), 7.26 (t, J=7.5 Hz, 1H), 7.12 (d, J=7.6 Hz, 2H), 6.38 (s, 1H), 5.12 (dd, J=10.6, 4.2 Hz, 1H), 4.45-4.15 (m, 2H), 3.72 (t, J=11.2 Hz, 1H), 3.31-3.18 (m, 2H), 2.26-1.82 (m, 8H), 1.67 (t, J=12.4 Hz, 1H), 1.36-1.24 (m, 1H), 1.24-1.09 (m, 1H), 0.73 (d, J=6.6 Hz, 3H), 0.56-0.49 (m, 2H), 0.49-0.40 (m, 2H), 0.21 (d, J=6.2 Hz, 3H). ESI-MS m/z calc. 560.2457, found 561.1 (M+1)⁺; Retention time: 2.06 minutes (LC method A).

### Example 90: Preparation of Compound 110

### Step 1: tert-Butyl 2,2-dimethyl-4-oxo-pyrrolidine-1-carboxylate

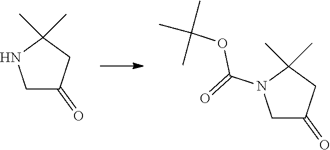

Di-tert-butyl dicarbonate (22.9 g, 24.11 mL, 104.9 mmol) was added to a solution of 5,5-dimethylpyrrolidin-3-one (hydrochloride salt) (13.08 g, 87.42 mmol), triethylamine (17.71 g, 24.4 mL, 175.0 mmol) and DMAP (1.1 g, 9.004 mmol in dichloromethane (325 mL) and reaction mixture was stirred at room temperature overnight. Reaction mixture was washed with 1N HCl (300 mL) and aqueous layer was extracted with dichloromethane (2×250 mL). The organic layers were combined, washed with 5% sodium bicarbonate (250 mL) and brine (150 mL), dried over sodium sulfate and concentrated under reduced pressure to afford tert-butyl 2,2-dimethyl-4-oxo-pyrrolidine-1-carboxylate (18.5 g, 99%) as a white solid. ¹H NMR (300 MHz, CDCl₃) δ 1.33-1.66 (m, 15H), 2.51 (s, 2H), 3.85 (br. s., 2H). [M-C₄H₈]+=158.2, Retention time=1.91 min, LC method K.

### Step 2: tert-Butyl 4-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-2,2-dimethyl-pyrrolidine-1-carboxylate, diastereomer 1 and diastereomer 2

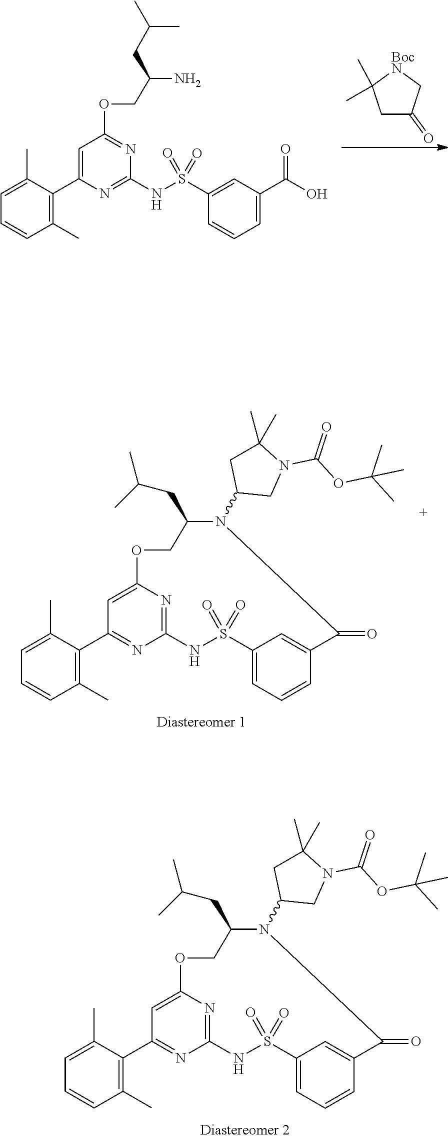 [SMILES: Cc1cccc(C)c1-c1cc(OC[C@H](N)CC(C)C)nc(NS(=O)(=O)c2cccc(C(=O)O)c2)n1 ; CC(C)(C)OC(=O)N1CC(=O)CC1(C)C ; Cc1cccc(C)c1-c1cc2nc(n1)NS(=O)(=O)c1cccc(c1)C(=O)N(C1CN(C(=O)OC(C)(C)C)C(C)(C)C1)[C@H](CC(C)C)CO2 ; Cc1cccc(C)c1-c1cc2nc(n1)NS(=O)(=O)c1cccc(c1)C(=O)N(C1CN(C(=O)OC(C)(C)C)C(C)(C)C1)[C@H](CC(C)C)CO2]

3-[[4-[(2R)-2-Amino-4-methyl-pentoxy]-6-(2,6-dimethylphenyl)pyrimidin-2-yl]sulfamoyl]benzoic acid (hydrochloride salt) (150 mg, 0.2803 mmol) was combined with the {tert}-butyl 2,2-dimethyl-4-oxo-pyrrolidine-1-carboxylate (90 mg, 0.4220 mmol) in DCM (1 mL) and stirred for 15 minutes at room temperature. Sodium triacetoxyborohydride (180 mg, 0.8493 mmol) was added and the reaction was stirred for an additional hour at room temperature. A second portion of sodium triacetoxyborohydride (180 mg, 0.8493 mmol) was added, and the reaction was stirred for an additional 2 hours. An additional portion of {tert}-butyl 2,2-dimethyl-4-oxo-pyrrolidine-1-carboxylate (90 mg, 0.4220 mmol) was added at this point, followed by an additional portion of sodium triacetoxyborohydride (180 mg, 0.8493 mmol) and the reaction was stirred for an additional 5 hours. The reaction mixture was then partitioned between 1M HCl and ethyl acetate. The layers were separated, and the aqueous was extracted an additional 3× ethyl acetate. The combined organics were washed with brine, dried over sodium sulfate and concentrated. The resulting reductive amination product was combined with COMU (360 mg, 0.8406 mmol) in DMF (15.00 mL) and DIPEA (400 μL, 2.296 mmol) was added by syringe. The reaction mixture was stirred for the indicated time at room temperature, then was partitioned between 1M HCl and ethyl acetate. The layers were separated and the aqueous was extracted an additional 3×ethyl acetate. The combined organics were washed with brine, dried over sodium sulfate and concentrated. was dissolved in 1:1 methanol DMSO, filtered, and purified by reverse phase HPLC (1-99% ACN in water HCl modifier, 30 min run, initially shallow gradient) to give two products: diastereomer 1, peak 1, tert-butyl 4-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-2,2-dimethyl-pyrrolidine-1-carboxylate (16 mg, 8%), ESI-MS m/z calc. 677.3247, found 678.5 (M+1)⁺, Retention time: 0.82 minutes (LC method D); and diastereomer 2, tert-butyl 4-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-2,2-dimethyl-pyrrolidine-1-carboxylate (7 mg, 4%), ESI-MS m/z calc. 677.3247, found 678.5 (M+1)⁺, Retention time: 0.84 minutes (LC method D);

### Step 3: (11R)-6-(2,6-Dimethylphenyl)-12-(5,5-dimethylpyrrolidin-3-yl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1

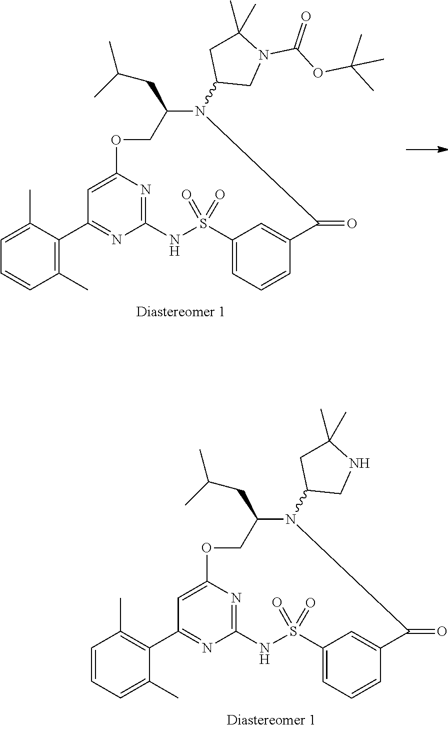 [SMILES: Cc1cccc(C)c1-c1cc2nc(n1)NS(=O)(=O)c1cccc(c1)C(=O)N(C1CN(C(=O)OC(C)(C)C)C(C)(C)C1)[C@H](CC(C)C)CO2 ; Cc1cccc(C)c1-c1cc2nc(n1)NS(=O)(=O)c1cccc(c1)C(=O)N(C1CNC(C)(C)C1)[C@H](CC(C)C)CO2]

tert-butyl 4-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-2,2-dimethyl-pyrrolidine-1-carboxylate (diastereomer 1, 16 mg, 0.02360 mmol) was combined in DCM (0.2 mL) with HCl (0.1 mL of 4 M, 0.4000 mmol) and stirred at room temperature for 30 minutes. Solvent was evaporated, hexanes were added, and the reactions were evaporated a second time to give (11R)-6-(2,6-dimethylphenyl)-12-(5,5-dimethylpyrrolidin-3-yl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (diastereomer 1, 14 mg, 97%). ESI-MS m/z calc. 577.2723, found 578.4 (M+1)⁺; Retention time: 0.49 minutes; LC method D.

### Step 4: Propan-2-yl 4-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]-2,2-dimethylpyrrolidine-1-carboxylate (Compound 110)

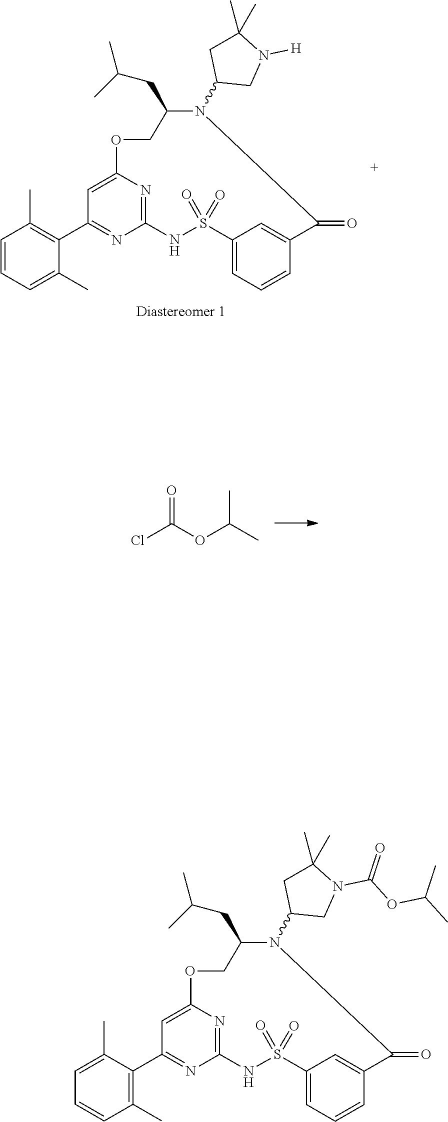

(11R)-6-(2,6-dimethylphenyl)-12-(5,5-dimethylpyrrolidin-3-yl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (diastereomer 1, 7 mg, 0.0114 mmol) was combined in dichloromethane (0.5 mL) with isopropyl chloroformate (approximately 11.4 μL of 2 M, 0.0228 mmol), and triethylamine (approximately 7.94 μL, 0.057 mmol) was added. The reaction was stirred for 30 minutes at room temperature. It was then quenched with several drops of 1M HCl, partially concentrated, diluted with 1:1 methanol/DMSO, filtered and purified by reverse phase HPLC (1-99% ACN in water, HCl modifier, 15 min run) to give propan-2-yl 4-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]-2,2-dimethylpyrrolidine-1-carboxylate (3.9 mg, 60%). ESI-MS m/z calc. 663.3091, found 664.5 (M+1)⁺; Retention time: 1.99 minutes; LC method A.

### Example 91: Preparation of Compound 111

### Step 1: (11R)-6-(2,6-Dimethylphenyl)-12-(5,5-dimethylpyrrolidin-3-yl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 2

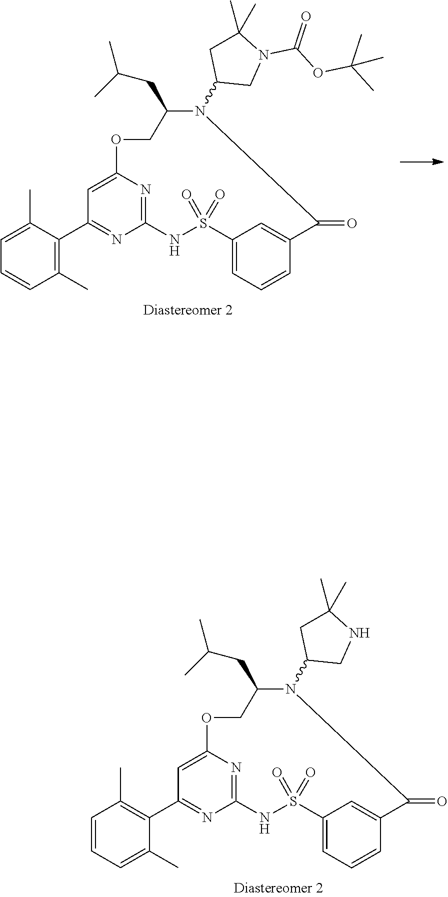 [SMILES: Cc1cccc(C)c1-c1cc2nc(n1)NS(=O)(=O)c1cccc(c1)C(=O)N(C1CN(C(=O)OC(C)(C)C)C(C)(C)C1)[C@H](CC(C)C)CO2 ; Cc1cccc(C)c1-c1cc2nc(n1)NS(=O)(=O)c1cccc(c1)C(=O)N(C1CNC(C)(C)C1)[C@H](CC(C)C)CO2]

tert-Butyl 4-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-2,2-dimethyl-pyrrolidine-1-carboxylate (diastereomer 2, 7 mg, 0.02360 mmol) was combined in DCM (0.2 mL) with HCl (0.1 mL of 4 M, 0.4000 mmol) and stirred at room temperature for 30 minutes. Solvent was evaporated, hexanes were added, and the reactions were evaporated a second time to give (11R)-6-(2,6-dimethylphenyl)-12-(5,5-dimethylpyrrolidin-3-yl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (diastereomer 2, 6 mg, 94%). ESI-MS m/z calc. 577.2723, found 578.4 (M+1)⁺; Retention time: 0.54 minutes; LC method D.

### Step 2: Propan-2-yl 4-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]-2,2-dimethylpyrrolidine-1-carboxylate (Compound 111)

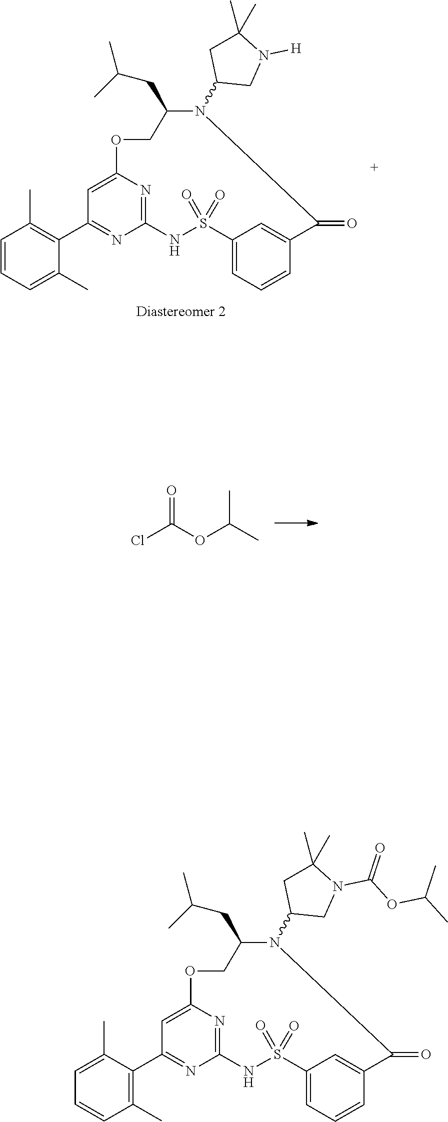

(11R)-6-(2,6-dimethylphenyl)-12-(5,5-dimethylpyrrolidin-3-yl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (diastereomer 2, 6 mg, 0.009769 mmol) was combined in dichloromethane (0.5 mL) with isopropyl chloroformate (approximately 9.770 μL of 2 M, 0.01954 mmol), and triethylamine (approximately 4.943 mg, 6.809 μL, 0.04885 mmol) was added. The reaction was stirred for 30 minutes at room temperature. It was then quenched with several drops of 1M HCl, partially concentrated, diluted with 1:1 methanol/DMSO, filtered and purified by reverse phase HPLC (1-99% ACN in water, HCl modifier, 15 min run) to give propan-2-yl 4-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]-2,2-dimethylpyrrolidine-1-carboxylate (3.9 mg, 60%). ESI-MS m/z calc. 663.3091, found 664.5 (M+1)⁺; Retention time: 2.06 minutes; LC method A.

### Example 92: Preparation of (11R)-6-(2,6-dimethylphenyl)-12-(3-hydroxycyclobutyl)-3,11-bis(2-methylpropyl)-9-oxa-2λ⁶-thia-3,5,12,19-tetraazatricyclo[12.3.1.14,8]nonadeca-1(18),4(19),5,7,14,16-hexaene-2,2,13-trione

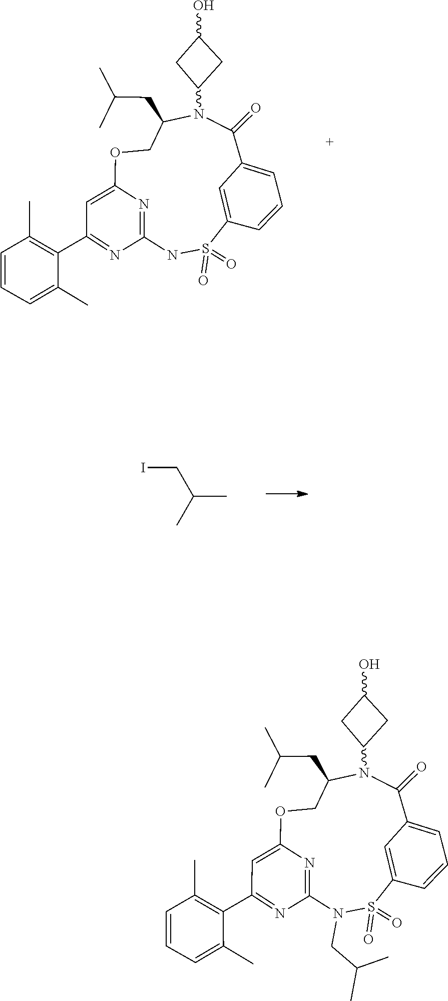

In a 4 mL vial, to a stirred solution of (11R)-6-(2,6-dimethylphenyl)-12-(3-hydroxycyclobutyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (30 mg, 0.05448 mmol) in anhydrous DMF (0.5 mL) was added cesium carbonate (72 mg, 0.2210 mmol), followed by addition of a solution of 1-iodo-2-methyl-propane (16 mg, 0.08695 mmol) in anhydrous DMF (0.1 mL). The vial was sparged with nitrogen for 30 s, then the capped vial was stirred at 40° C. 14 h. Glacial acetic acid (50 μL, 0.8792 mmol) was added slowly and diluted with DMSO (1 mL), micro-filtered and purified by reverse-phase HPLC (Cis column, 1-99% acetonitrile in water over 15 min, HCl as modifier) to furnish (11R)-6-(2,6-dimethylphenyl)-12-(3-hydroxycyclobutyl)-3,11-bis(2-methylpropyl)-9-oxa-2λ⁶-thia-3,5,12,19-tetraazatricyclo[12.3.1.14,8]nonadeca-1(18),4(19),5,7,14,16-hexaene-2,2,13-trione (3.6 mg, 10%) as white solid. ESI-MS m/z calc. 606.2876, found 607.1 (M+1)⁺; Retention time: 1.83 minutes (LC method A).

### Example 93: Preparation of Compound 113

### Step 1: tert-Butyl 2-[[(1S)-1-(hydroxymethyl)-3-methyl-butyl]amino]-7-azaspiro[3.5]nonane-7-carboxylate

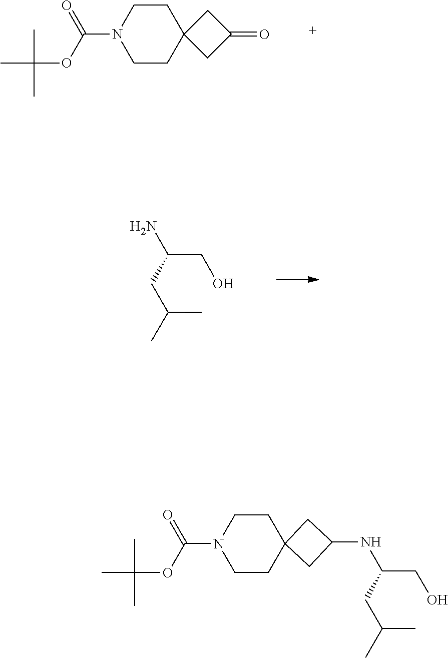

A solution of tert-butyl 2-oxo-7-azaspiro[3.5]nonane-7-carboxylate (244 mg, 1.020 mmol) and (2S)-2-amino-4-methyl-pentan-1-ol (170 μL, 1.330 mmol) in DCE (2 mL) was stirred at room temperature for 20 min. The reaction mixture was cooled on an ice bath and sodium triacetoxyborohydride (640 mg, 3.020 mmol) was added in two equal portions 20 min apart. After stirring for 10 min in an ice bath, the ice bath was removed, and the reaction mixture stirred at room temperature for 24 hours. The reaction mixture was cooled to 0° C. on an ice water bath and treated with HCl (4.1 mL of 1 M, 4.100 mmol) over 5 min then stirred at this temp for 10 min. Water was added (5 mL), then solid potassium carbonate (1.46 g, 10.56 mmol) was added in portions over 5 min, the cooling bath removed and the reaction mixture poured into water and extracted with EtOAc (2×). Organics combined, washed with 2M aqueous potassium carbonate, brine, dried over sodium sulfate and evaporated to dryness to give tert-butyl 2-[[(1S)-1-(hydroxymethyl)-3-methyl-butyl]amino]-7-azaspiro[3.5]nonane-7-carboxylate (330 mg, 95%) as a clear oil. ESI-MS m/z calc. 340.27258, found 341.3 (M+1)⁺; Retention time: 0.44 minutes (LC method D).

### Step 2: tert-Butyl 2-[(11S)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate

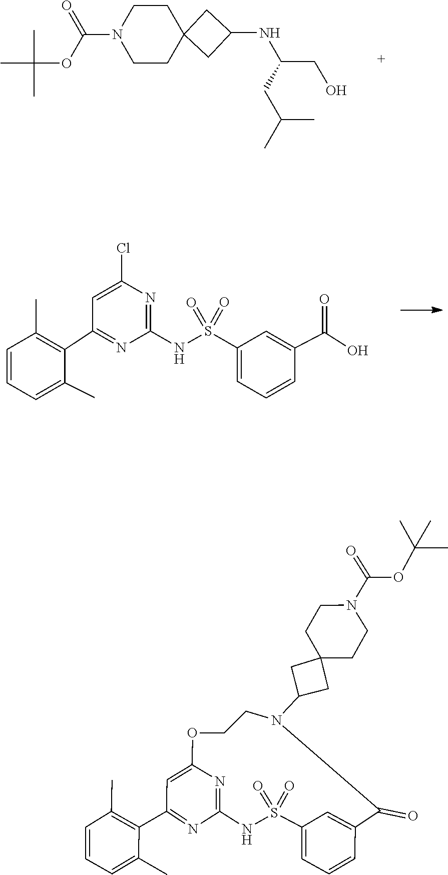 [SMILES: CC(C)C[C@@H](CO)NC1CC2(CCN(C(=O)OC(C)(C)C)CC2)C1 ; Cc1cccc(C)c1-c1cc(Cl)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CCN(C(=O)OC(C)(C)C)CC3)C1)CCO2]

To a solution of tert-butyl 2-[[(1S)-1-(hydroxymethyl)-3-methyl-butyl]amino]-7-azaspiro[3.5]nonane-7-carboxylate (300 mg, 0.8811 mmol) and 3-[[4-chloro-6-(2,6-dimethylphenyl)pyrimidin-2-yl]sulfamoyl]benzoic acid (395 mg, 0.9453 mmol) in THE (6 mL) at 0° C. was added NaOtBu (455 mg, 4.734 mmol) and the reaction mixture was stirred at room temperature for 2 hours. The reaction mixture was then added to a stirred solution of HATU (700 mg, 1.841 mmol) in DMF (10 mL) dropwise. DiPEA (767 μL, 4.403 mmol) was added and the reaction mixture stirred at room temperature for 16 hours. The reaction mixture was poured into water, the pH brought to pH˜5 with 1N HCl and extracted with EtOAc (3×). Organics were combined, washed with water and evaporated to dryness. Purification by column chromatography (24 g silica, 0-50% EtOAc in hexanes) gave tert-butyl 2-[(11S)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (110 mg, 18%) as a foam. ESI-MS m/z calc. 703.34033, found 704.6 (M+1)⁺; Retention time: 0.82 minutes (LC method D).

### Step 3: Methyl 2-[(11S)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (Compound 113)

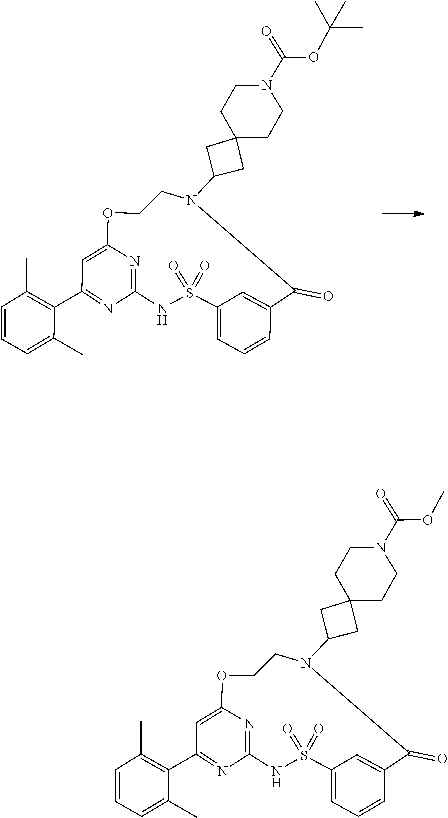 [SMILES: Cc1cccc(C)c1-c1cc2nc(n1)NS(=O)(=O)c1cccc(c1)C(=O)N(C1CC3(CCN(C(=O)OC(C)(C)C)CC3)C1)CCO2 ; COC(=O)N1CCC2(CC1)CC(N1CCOc3cc(-c4c(C)cccc4C)nc(n3)NS(=O)(=O)c3cccc(c3)C1=O)C2]

To a solution of tert-butyl 2-[(11S)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (110 mg, 0.1563 mmol) in DCM (2 mL) was added HCl (4M in dioxane) (400 μL of 4 M, 1.600 mmol) and the reaction mixture stirred for 2 hours. The reaction mixture was evaporated to dryness then taken up in DCM (2 mL). To the cooled solution (0° C.) was added TEA (75 μL, 0.5381 mmol) followed by methyl chloroformate (11 μL, 0.1424 mmol), the cooling bath was removed, and the reaction mixture was stirred at room temperature for 2 hours. The reaction mixture was then evaporated, then taken up in 1:1 MeOH: DMSO, filtered and subjected to purification by HPLC (1-99% ACN in water (HCl modifier)) to give methyl 2-[(11S)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (40.5 mg, 39%) as a white solid. ESI-MS m/z calc. 661.2934, found 662.4 (M+1)⁺; Retention time: 1.86 minutes (LC method A).

### Example 94: Preparation of Compound 114 and Compound 115

### Step 1: Methyl 2-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]spiro[3.3]heptane-6-carboxylate

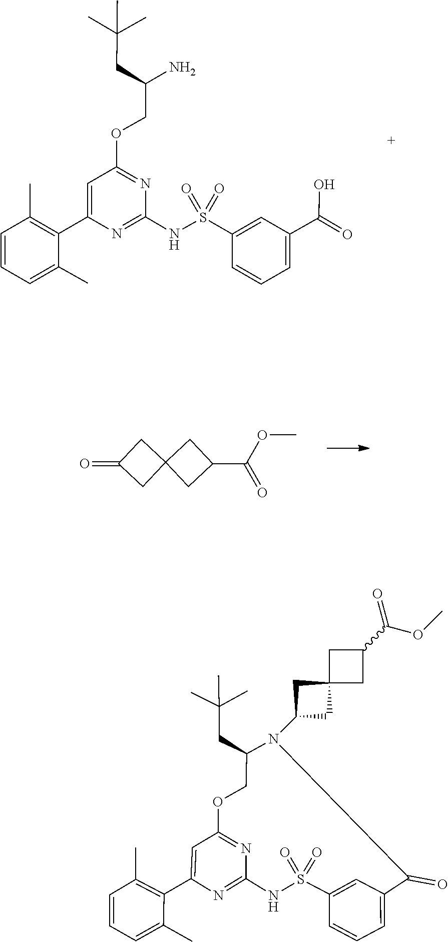

3-[[4-[(2R)-2-Amino-4,4-dimethyl-pentoxy]-6-(2,6-dimethylphenyl)pyrimidin-2-yl]sulfamoyl]benzoic acid (hydrochloride salt) (1 g, 1.821 mmol) was combined with methyl 2-oxospiro[3.3]heptane-6-carboxylate (460 mg, 2.735 mmol) in DCM (4 mL) and stirred for 20 minutes at room temperature. Sodium triacetoxyborohydride (1.25 g, 5.898 mmol) was added and the reaction was stirred for an additional hour. Additional sodium triacetoxyborohydride (400 mg, 1.887 mmol) was added, and after an additional 90 minutes at room temperature the reaction mixture was partitioned between 1M HCl and ethyl acetate. The layers were separated and the aqueous was extracted an additional 3× with ethyl acetate. The combined organics were washed with brine, dried over sodium sulfate, and concentrated. The material was used in the next step without further purification. 3-[[4-(2,6-dimethylphenyl)-6-[(2R)-2-[(6-methoxycarbonylspiro[3.3]heptan-2-yl)amino]-4,4-dimethyl-pentoxy]pyrimidin-2-yl]sulfamoyl]benzoic acid ESI-MS m/z calc. 664.2931, found 665.6 (M+1)⁺; Retention time: 1.15 minutes (LC method A).

A solution of the product above in DMF (5 mL) was added dropwise to a stirring solution of COMU (1.7 g, 3.969 mmol) and DIPEA (1.6 mL, 9.186 mmol) in DMF (35 mL) over 5 minutes. The reaction mixture was stirred for 16 hours at room temperature, after which it was partially concentrated under reduced pressure then partitioned between 1M HCl and ethyl acetate. The layers were separated, and the aqueous was extracted an additional 2×with ethyl acetate. The combined organics were washed with brine, dried over sodium sulfate and concentrated. The resulting crude material was purified by chromatography on silica gel, eluting with a 0-100% ethyl acetate in hexanes gradient to give a slightly yellow solid, methyl 2-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]spiro[3.3]heptane-6-carboxylate (597 mg, 51%). ESI-MS m/z calc. 646.28253, found 647.7 (M+1)⁺; Retention time: 1.98 minutes (LC method A).

### Step 2: 6-[(11R)-6-(2,6-Dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]-N,N-dimethylspiro[3.3]heptane-2-carboxamide, diastereomer 1 (Compound 114), and 6-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]-N,N-dimethylspiro[3.3]heptane-2-carboxamide, diastereomer 2 (Compound 115)

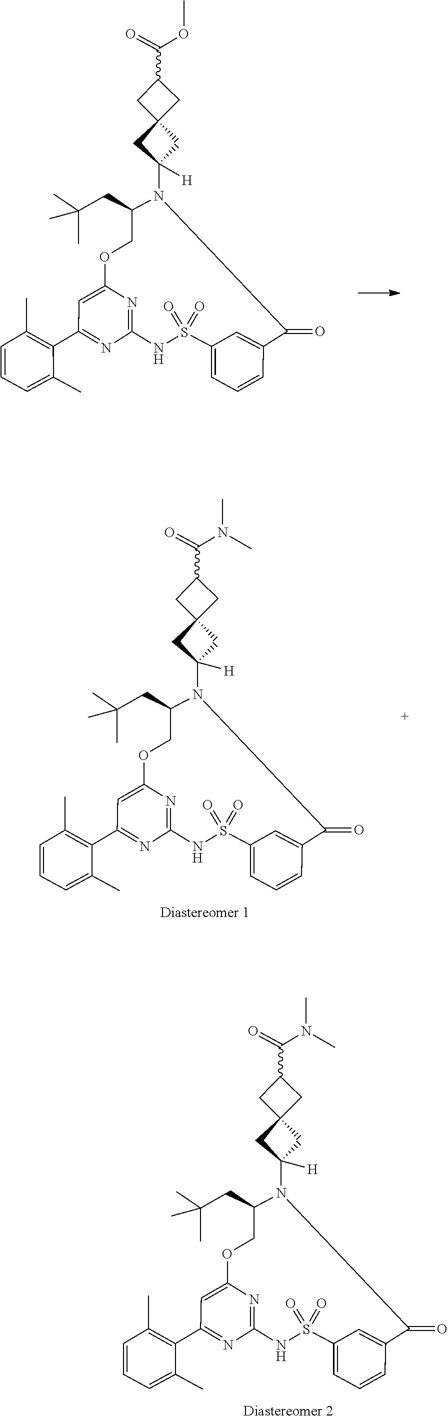

A vial was charged with methyl 6-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]spiro[3.3]heptane-2-carboxylate (27 mg, 0.04174 mmol), THE (400 μL), LiOH (18 mg, 0.7516 mmol) and water (55 μL) and it was stirred at room temperature for 1.5 hours. More LiOH (15 mg, 0.6264 mmol) was added and the mixture was stirred for an additional 35 min. The reaction was diluted with water and DMSO to a total volume of 2 mL. The solution was microfiltered through syringe filter disc and purified by reverse phase preparative HPLC (C₁₈) using a gradient of acetonitrile in water (1 to 99% over 15 min) and HCl as a modifier to give 6-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]spiro[3.3]heptane-2-carboxylic acid (22 mg, 83%) as a white solid (1:1 mixture of diastereomers). ESI-MS m/z calc. 632.26685, found 633.25 (M+1)⁺; Retention time: 1.68 minutes. second isomer found 633.25 (M+1)⁺, retention times: 1.72 minutes (ratio 1:1), (LC method A).

The crude acid was treated at room temperature with HATU (37 mg, 0.09731 mmol), N-methylmethanamine (hydrochloride salt) (36 mg, 0.4415 mmol), anhydrous DMF (0.5 mL) and DIEA (110 μL, 0.6315 mmol) for 3 h. The reaction was diluted with DMSO (1 mL). The solution was microfiltered through a syringe filter disc and purified by reverse phase preparative HPLC (C₁₈) using a gradient of acetonitrile in water (1 to 99% over 15 min) and HCl as a modifier (poor separation of isomers). After evaporation of the solvents, the mixture was purified using a shallower gradient. 10-50% over 20 min, 50-100% over 5 min of MeCN in water/HCl. The two impure fractions were purified a second time using a gradient 10-25% over 10 min, 25-60% over 30 min of MeCN in water/HCl. Evaporation provided both separated isomers: A more polar, peak 1, diastereomer 1,6-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]-N,N-dimethylspiro[3.3]heptane-2-carboxamide (4.1 mg, 30%). ESI-MS m/z calc. 659.31415, found 660.71 (M+1)⁺; Retention time: 1.75 minutes (LC method A); and a less polar, peak 2, diastereomer 2,6-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]-N,N-dimethylspiro[3.3]heptane-2-carboxamide (3.2 mg, 23%). ESI-MS m/z calc. 659.31415, found 660.82 (M+1)⁺; Retention time: 1.79 minutes (LC method A).

### Example 95: Preparation of Compound 116 and Compound 117

### Step 1: (11R)-6-(2,6-dimethylphenyl)-11-(2,2-dimethylpropyl)-12-[6-(2-hydroxypropan-2-yl)spiro[3.3]heptan-2-yl]-9-oxa-2λ⁶-thia-3,5,12,19-tetraazatricyclo[12.3.1.14,8]nonadeca-1(17),4(19),5,7,14(18),15-hexaene-2,2,13-trione, diastereomer 1 (Compound 116), and (11R)-6-(2,6-dimethylphenyl)-11-(2,2-dimethylpropyl)-12-[6-(2-hydroxypropan-2-yl)spiro[3.3]heptan-2-yl]-9-oxa-2%⁶-thia-3,5,12,19-tetraazatricyclo[12.3.1.14,8]nonadeca-1(17),4(19),5,7,14(18),15-hexaene-2,2,13-trione, diastereomer 2 (Compound 117)

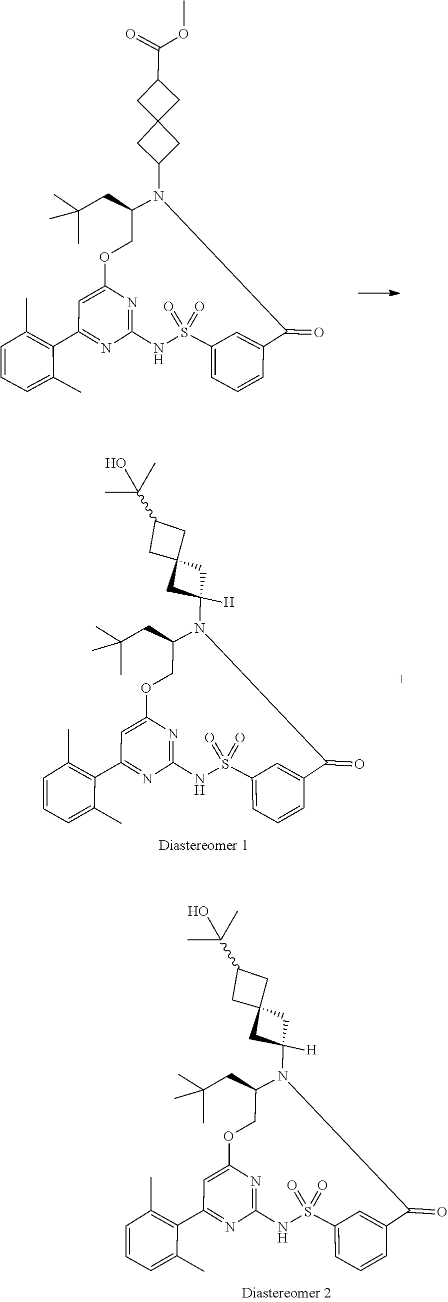

A 4 mL vial was charged under nitrogen with methyl 6-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]spiro[3.3]heptane-2-carboxylate (38 mg, 0.05875 mmol) (isomer ratio 1:1), anhydrous THE (0.4 mL) and the solution was cooled down in an ice bath. Bromo(methyl)magnesium (0.06 mL of 3 M, 0.1800 mmol, 3M solution in diethyl ether) was added dropwise. The reaction mixture was stirred in the ice bath for 9 min, then it was stirred at room temperature for one hour. The mixture was cooled down in ice and quenched by adding an aqueous saturated solution of ammonium chloride (2 mL). The product was extracted with EtOAc (3×2 mL). The combined extracts were dried over sodium sulfate and the solvents were evaporated. The residue was dissolved in DMSO (2 mL). The solution was microfiltered through a syringe filter disc and purified by reverse phase preparative HPLC (C₁₈) using a gradient of acetonitrile in water (1 to 99% over 15 min) and HCl as a modifier, providing two fractions, each enriched in one of the diastereomers. After evaporation, each fraction was purified a second time in the same conditions with the following gradient: 10-60% over 10 min, 60-70% over 10 min, 70-100% over 5 min of acetonitrile in water/HCl, to give a peak 1, more polar isomer, diastereomer 1, (11R)-6-(2,6-dimethylphenyl)-11-(2,2-dimethylpropyl)-12-[6-(2-hydroxypropan-2-yl)spiro[3.3]heptan-2-yl]-9-oxa-2λ⁶-thia-3,5,12,19-tetraazatricyclo[12.3.1.14,8]nonadeca-1(17),4(19),5,7,14(18),15-hexaene-2,2,13-trione (6 mg, 32%). ESI-MS m/z calc. 646.3189, found 647.8 (M+1)⁺; Retention time: 1.92 minutes (LC method A), ¹H NMR (400 MHz, DMSO-d₆) δ 13.50-11.69 (broad m, 1H), 8.41 (s, 1H), 7.91 (s, 1H), 7.65 (s, 2H), 7.25 (s, 1H), 7.12 (s, 2H), 6.39 (s, 1H), 5.07 (dd, J=10.7, 4.3 Hz, 1H), 4.38-4.21 (m, 1H), 3.98 (s, 1H), 3.86 (p, J=8.7 Hz, 1H), 3.66 (br s, 1H), 2.92 (dt, J=14.1, 9.8 Hz, 2H), 2.36-2.25 (m, 1H), 2.24-1.84 (m, 11H), 1.80 (t, J=9.4 Hz, 1H), 1.59 (dd, J=15.1, 8.3 Hz, 1H), 1.37 (d, J=14.9 Hz, 1H), 0.97 (d, J=2.5 Hz, 6H), 0.49 (s, 9H); and a peak 2, less polar isomer, diastereomer 2 (11R)-6-(2,6-dimethylphenyl)-11-(2,2-dimethylpropyl)-12-[6-(2-hydroxypropan-2-yl)spiro[3.3]heptan-2-yl]-9-oxa-2λ⁶-thia-3,5,12,19-tetraazatricyclo[12.3.1.14,8]nonadeca-1(17),4(19),5,7,14(18),15-hexaene-2,2,13-trione (10 mg, 53%). ESI-MS m/z calc. 646.3189, found 647.77 (M+1)⁺; Retention time: 1.97 minutes (LC method A).

### Example 96: Preparation of Compound 118, Compound 119, and Compound 120

### Step 1: (11R)-12-{6-Aminospiro[3.3]heptan-2-yl}-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione (Compound 118)

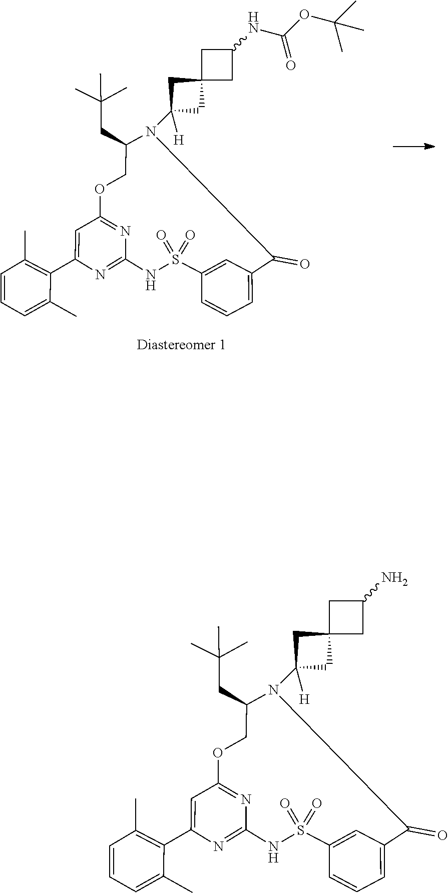

In a round bottom flask, tert-butyl N-[2-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (diastereomer 1, 7.8954 g, 11.22 mmol) was dissolved in DCM (35 mL) along with HCl (28.05 mL of 4 M, 112.2 mmol)(4M in Dioxane). The reaction mixture was stirred at rt for 1 h. then evaporated to dryness. The solid material was slurried in a mixture of 50% ethyl acetate/hexanes and filtered. The product was recovered as a white solid (HCl salt). (11R)-12-{6-Aminospiro[3.3]heptan-2-yl}-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione (hydrochloride salt) (7.383 g, 103%). ¹H NMR (400 MHz, Methanol-d₄) δ 8.57 (d, J=2.2 Hz, 1H), 8.09-8.00 (m, 1H), 7.78-7.67 (m, 2H), 7.30 (t, J=7.6 Hz, 1H), 7.16 (d, J=7.6 Hz, 2H), 6.30 (s, 1H), 5.29 (dd, J=10.8, 4.3 Hz, 1H), 4.25 (t, J=11.2 Hz, 1H), 4.06 (h, J=8.5 Hz, 1H), 3.85 (ddd, J=12.0, 8.0, 4.3 Hz, 1H), 3.79-3.66 (m, 2H), 3.28 (t, J=9.7 Hz, 2H), 3.15 (dd, J=11.4, 8.7 Hz, 1H), 2.69-2.49 (m, 3H), 2.43-2.33 (m, 1H), 2.29 (ddd, J=11.6, 8.6, 2.8 Hz, 2H), 2.09 (s, 1H), 1.69 (dd, J=15.3, 8.2 Hz, 1H), 1.54 (d, J=15.1 Hz, 1H), 1.40-1.29 (m, 2H), 0.96-0.85 (m, 2H), 0.61 (s, 9H). ESI-MS m/z calc. 603.2879, found 604.6 (M+1)⁺; Retention time: 1.31 minutes (LC method A).

### Step 2: (11R)-6-(2,6-Dimethylphenyl)-11-(2,2-dimethylpropyl)-2,2-dioxo-12-(2-oxospiro[3.3]heptan-6-yl)-9-oxa-2λ⁶-thia-3,5,12,19-tetrazatricyclo[12.3.1.14,8]nonadeca-1(18),4,6,8(19),14,16-hexaen-13-one

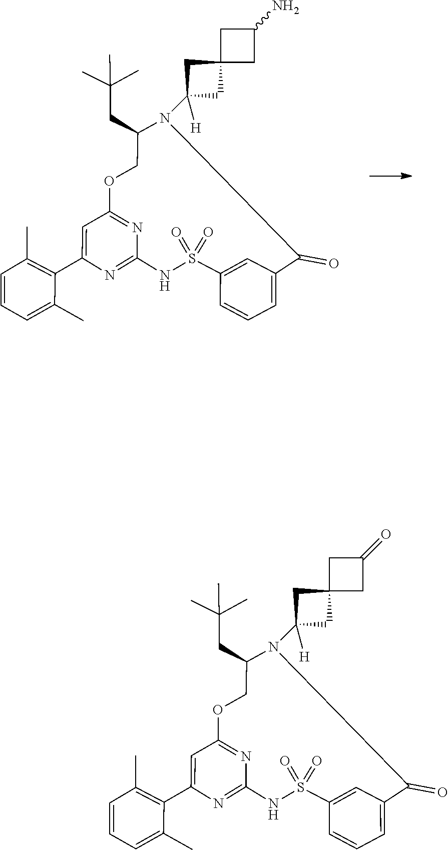

In a reaction vial, (11R)-12-{6-aminospiro[3.3]heptan-2-yl}-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione (hydrochloride salt) (603.8 mg, 0.9431 mmol) was mixed with diisopropylethylamine (191.6 μL, 1.100 mmol) in methylene chloride (10 mL). To the reaction, a mixture of (1:1) potassium permanganate (500 mg, 3.164 mmol):copper sulfate pentahydrate (500 mg, 2.003 mmol) (equal amounts by weight of KMnO₄ and CuSO₄ were ground up in a mortar until homogeneous) was added and the reaction was stirred at 40° C. overnight. The reaction mixture was evaporated to dryness and diluted with 1N HCl/ethyl acetate. The mixture was filtered through Celite and the organic layer was collected. The aqueous layer was extracted with additional ethyl acetate and all the organic layers were combined. The combined organic layer was washed with saturated NaCl solution, isolated, dried over anhydrous sodium sulfate, filtered, and evaporated to dryness. The crude material was purified by column chromatography on silica using 0-15% MeOH/DCM gradient. The product was isolated as a light yellow solid. (11R)-6-(2,6-dimethylphenyl)-11-(2,2-dimethylpropyl)-2,2-dioxo-12-(2-oxospiro[3.3]heptan-6-yl)-9-oxa-2λ⁶-thia-3,5,12,19-tetrazatricyclo[12.3.1.14,8]nonadeca-1(18),4,6,8(19),14,16-hexaen-13-one (146 mg, 23%) ESI-MS m/z calc. 602.2563, found 603.4 (M+1)⁺; Retention time: 1.82 minutes (LC method A).

### Step 3: (11R)-6-(2,6-Dimethylphenyl)-11-(2,2-dimethylpropyl)-12-(2-hydroxy-2-methyl-spiro[3.3]heptan-6-yl)-2,2-dioxo-9-oxa-2λ⁶-thia-3,5,12,19-tetrazatricyclo[12.3.1.14,8]nonadeca-1(18),4,6,8(19),14,16-hexaen-13-one, diastereomer 1 (Compound 119), and (11R)-6-(2,6-dimethylphenyl)-11-(2,2-dimethylpropyl)-12-(2-hydroxy-2-methyl-spiro[3.3]heptan-6-yl)-2,2-dioxo-9-oxa-2λ⁶-thia-3,5,12,19-tetrazatricyclo[12.3.1.14,8]nonadeca-1(18),4,6,8(19),14,16-hexaen-13-one, diastereomer 2 (Compound 120)

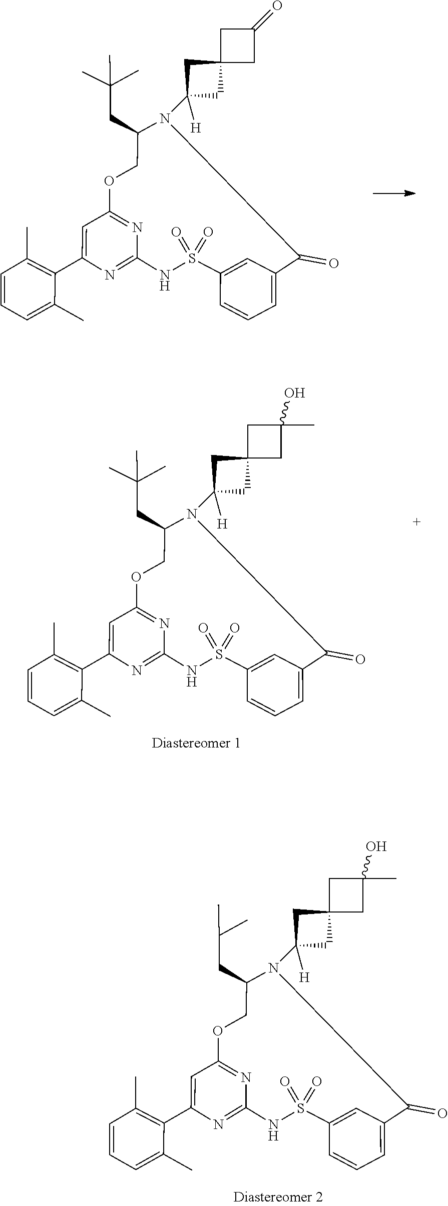

In a reaction vial, (11R)-6-(2,6-dimethylphenyl)-11-(2,2-dimethylpropyl)-2,2-dioxo-12-(2-oxospiro[3.3]heptan-6-yl)-9-oxa-2λ⁶-thia-3,5,12,19-tetrazatricyclo[12.3.1.14,8]nonadeca-1(18),4,6,8(19),14,16-hexaen-13-one (52 mg, 0.08627 mmol) was dissolved in THE (1 mL) and cooled to 0° C. To the reaction, bromo(methyl)magnesium (86.3 μL of 3 M, 0.2589 mmol) was added and the reaction was allowed to stir at 0° C. for 1 h. then more bromo(methyl)magnesium (30 μL, 0.2591 mmol) was added. Stirring was continued at 0° C. for 40 min then the reaction was quenched with 1N HCl and extracted with ethyl acetate. The crude material was purified by preparative HPLC using 35-70% water/ACN gradient with HCl modifier. Two isomers were isolated: Peak 1, diastereomer 1, (11R)-6-(2,6-dimethylphenyl)-11-(2,2-dimethylpropyl)-12-(2-hydroxy-2-methyl-spiro[3.3]heptan-6-yl)-2,2-dioxo-9-oxa-2λ⁶-thia-3,5,12,19-tetrazatricyclo[12.3.1.14,8]nonadeca-1(18),4,6,8(19),14,16-hexaen-13-one (6.9 mg, 23%), ESI-MS m/z calc. 618.2876, found 619.3 (M+1)⁺; Retention time: 1.76 minutes (LC method A); and Peak 2, diastereomer 1, (11R)-6-(2,6-dimethylphenyl)-11-(2,2-dimethylpropyl)-12-(2-hydroxy-2-methyl-spiro[3.3]heptan-6-yl)-2,2-dioxo-9-oxa-2λ⁶-thia-3,5,12,19-tetrazatricyclo[12.3.1.14,8]nonadeca-1(18),4,6,8(19),14,16-hexaen-13-one (6.7 mg, 23%) ESI-MS m/z calc. 618.2876, found 619.6 (M+1)⁺; Retention time: 1.81 minutes (LC method A).

### Example 97: Preparation of Compound 121 and Compound 122

### Step 1: (11R)-6-(2,6-dimethylphenyl)-11-(2,2-dimethylpropyl)-12-(2-hydroxyspiro[3.3]heptan-6-yl)-2,2-dioxo-9-oxa-2λ⁶-thia-3,5,12,19-tetrazatricyclo[12.3.1.14,8]nonadeca-1(18),4,6,8(19),14,16-hexaen-13-one, diastereomer 1 (Compound 121), and (11R)-6-(2,6-dimethylphenyl)-11-(2,2-dimethylpropyl)-12-(2-hydroxyspiro[3.3]heptan-6-yl)-2,2-dioxo-9-oxa-2λ⁶-thia-3,5,12,19-tetrazatricyclo[12.3.1.14,8]nonadeca-1(18),4,6,8(19),14,16-hexaen-13-one, diastereomer 2 (Compound 122)

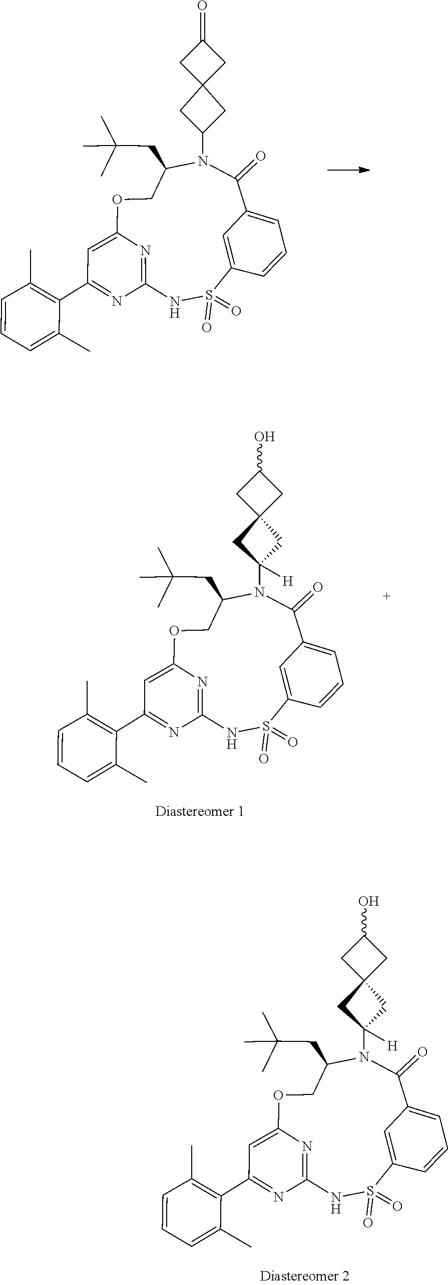

To a stirred solution of (11R)-6-(2,6-dimethylphenyl)-11-(2,2-dimethylpropyl)-12-{6-oxospiro[3.3]heptan-2-yl}-9-oxa-2λ⁶-thia-3,5,12,19-tetraazatricyclo[12.3.1.14,8]nonadeca-1(17),4(19),5,7,14(18),15-hexaene-2,2,13-trione (42 mg, 0.06062 mmol) in anhydrous methanol (1 mL) was added sodium borohydride (10 mg, 0.2643 mmol) slowly at 0-5° C. (ice-water bath) under nitrogen. Stirring was continued for 30 min at that temperature, then the reaction was quenched with water (1 mL) and brine (1 mL) and extracted with EtOAc (3×10 mL). The combined organics were concentrated under reduced pressure and the crude was taken up in DMSO (1.5 mL), micro-filtered, and purified by (reverse-phase HPLC, 18 column, 1-99% acetonitrile in water over 15 min, HCl as modifier). The pure fractions were dried in Genevac to give (11R)-6-(2,6-dimethylphenyl)-11-(2,2-dimethylpropyl)-12-(2-hydroxyspiro[3.3]heptan-6-yl)-2,2-dioxo-9-oxa-2λ⁶-thia-3,5,12,19-tetrazatricyclo[12.3.1.14,8]nonadeca-1(18),4,6,8(19),14,16-hexaen-13-one (mixture of isomers, 24 mg, 65%) as a white solid. ESI-MS m/z calc. 604.2719, found 605.3 (M+1)⁺; Retention time: 1.65 minutes (LC method A).

The mixture of isomers was subjected to chiral SFC using a ChiralPak IG (250×10 mm, 5 m column at 35° C., and a mobile phase comprised of 33% MeOH (no modifier) and 67% CO₂, with a flow of 10 mL/min, a concentration of 22 mg/mL in MeOH, an injection volume of 70 μL, at a pressure of 191 bar and using a 210 nm wavelength. Two product were isolated: Diastereomer 1, SFC Peak 1, (11R)-6-(2,6-dimethylphenyl)-11-(2,2-dimethylpropyl)-12-(2-hydroxyspiro[3.3]heptan-6-yl)-2,2-dioxo-9-oxa-2λ⁶-thia-3,5,12,19-tetrazatricyclo[12.3.1.14,8]nonadeca-1(18),4,6,8(19),14,16-hexaen-13-one (5.8 mg, 15%), 1H NMR (400 MHz, DMSO-d₆) δ 13.04 (s, 1H), 8.40 (s, 1H), 7.89 (s, 1H), 7.63 (s, 2H), 7.23 (s, 1H), 7.11 (s, 2H), 6.36 (s, 1H), 5.05 (dd, J=10.8, 4.3 Hz, 1H), 4.89 (d, J=6.2 Hz, 1H), 4.26 (s, 1H), 4.05-3.94 (m, 1H), 3.90 (t, J=8.6 Hz, 1H), 3.74-3.63 (m, 1H), 3.04 (t, J=9.8 Hz, 1H), 2.94 (t, J=9.6 Hz, 1H), 2.46-2.41 (m, 1H), 2.30-2.22 (m, 1H), 2.22-2.10 (m, 3H), 2.01-1.79 (m, 6H), 1.56 (dd, J=15.3, 8.2 Hz, 1H), 1.36 (d, J=14.8 Hz, 1H), 0.96-0.77 (m, 1H), 0.48 (s, 9H), ESI-MS m/z calc. 604.2719, found 605.3 (M+1)⁺; Retention time: 1.66 minutes (LC method A); and diastereomer 2, SFC Peak 2, (11R)-6-(2,6-dimethylphenyl)-11-(2,2-dimethylpropyl)-12-(2-hydroxyspiro[3.3]heptan-6-yl)-2,2-dioxo-9-oxa-2λ⁶-thia-3,5,12,19-tetrazatricyclo[12.3.1.14,8]nonadeca-1(18),4,6,8(19),14,16-hexaen-13-one (2.6 mg, 7%) ESI-MS m/z calc. 604.2719, found 605.2 (M+1)⁺; Retention time: 1.67 minutes (LC method A).

### Example 98: Preparation of Compound 123

### Step 1: tert-Butyl N-[6-[[(1R)-1-(hydroxymethyl)-3-methyl-butyl]amino]spiro[3.3]heptan-2-yl]carbamate

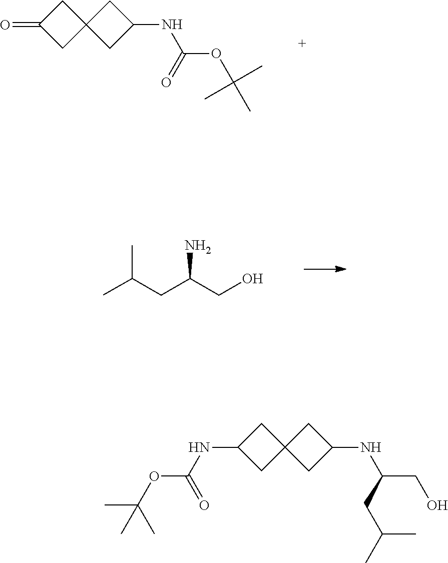

tert-Butyl N-(2-oxospiro[3.3]heptan-6-yl)carbamate (1.98 g, 8.789 mmol) was added to a solution of (2R)-2-amino-4-methyl-pentan-1-ol (1.347 g, 11.49 mmol) in anhydrous dichloromethane (6 mL) and stirred at room temperature for 1 h. sodium triacetoxyborohydride (5.87 g, 27.70 mmol) was added and the reaction was stirred an additional 48 h. HCl (35 mL of 1 M, 35.00 mmol) was added slowly and the reaction was stirred at room temperature an additional 1 h. The reaction was diluted with dichloromethane (5 mL) and a solution of potassium carbonate (15.6 g, 112.9 mmol) in H₂O (15 mL) was added and the organics were separated. The organics were washed with a solution of potassium carbonate (1.22 g, 8.827 mmol) plus water (10 mL). The organic layer was separated, dried with magnesium sulfate, then concentrated to give tert-butyl N-[6-[[(1R)-1-(hydroxymethyl)-3-methyl-butyl]amino]spiro[3.3]heptan-2-yl]carbamate (2.55 g, 89%) ESI-MS m/z calc. 326.25696, found 327.6 (M+1)⁺; Retention time: 0.46 minutes (LC method A).

### Step 2: 3-[[4-[(2R)-2-[[2-(tert-Butoxycarbonylamino)spiro[3.3]heptan-6-yl]amino]-4-methyl-pentoxy]-6-(2,6-dimethylphenyl)pyrimidin-2-yl]sulfamoyllbenzoic acid

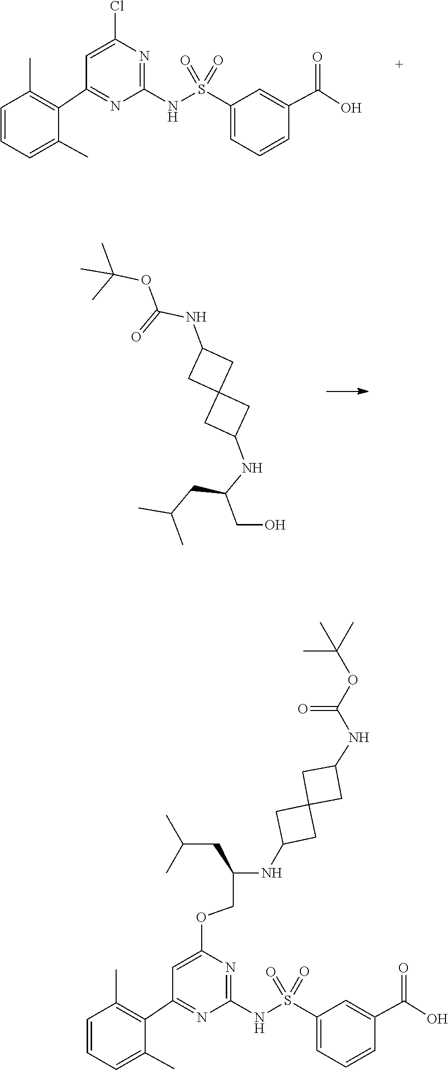 [SMILES: Cc1cccc(C)c1-c1cc(Cl)nc(NS(=O)(=O)c2cccc(C(=O)O)c2)n1 ; CC(C)C[C@H](CO)NC1CC2(CC(NC(=O)OC(C)(C)C)C2)C1 ; Cc1cccc(C)c1-c1cc(OC[C@@H](CC(C)C)NC2CC3(CC(NC(=O)OC(C)(C)C)C3)C2)nc(NS(=O)(=O)c2cccc(C(=O)O)c2)n1]

In a reaction vial, 3-[[4-chloro-6-(2,6-dimethylphenyl)pyrimidin-2-yl]sulfamoyl]benzoic acid (2.907 g, 6.957 mmol) and tert-butyl N-[6-[[(1R)-1-(hydroxymethyl)-3-methyl-butyl]amino]spiro[3.3]heptan-2-yl]carbamate (2.3 g, 7.045 mmol) were dissolved in THE (14.9 mL) then sodium tert-butoxide (2.75 g, 28.62 mmol) was added. The reaction was stirred for 2 h. at rt. An additional amount of sodium tert-butoxide (565.3 mg, 5.882 mmol) was added and stirring was continued for 30 min. The reaction mixture was then partitioned between ethyl acetate and a 1M HCl solution. The organics were separated, washed with brine, dried over sodium sulfate, and evaporated to dryness. The solid material was slurried in 50% ethyl acetate/hexanes then filtered to provide the product (HCl salt) as a light yellow solid 3-[[4-[(2R)-2-[[2-(tert-butoxycarbonylamino)spiro[3.3]heptan-6-yl]amino]-4-methyl-pentoxy]-6-(2,6-dimethylphenyl)pyrimidin-2-yl]sulfamoyl]benzoic acid (hydrochloride salt)(4.3053 g, 81%). ¹H NMR (400 MHz, DMSO-d₆) δ 8.47 (d, J=1.9 Hz, 1H), 8.15 (t, J=8.7 Hz, 2H), 7.71 (t, J=7.8 Hz, 1H), 7.26 (t, J=7.6 Hz, 1H), 7.13 (d, J=7.6 Hz, 2H), 7.10-7.03 (m, 1H), 6.37 (s, 1H), 4.34 (d, J=12.0 Hz, 1H), 4.22 (d, J=11.7 Hz, 1H), 3.80 (p, J=8.0 Hz, 1H), 3.67 (s, 1H), 2.36-2.09 (m, 6H), 2.01 (s, 4H), 1.89 (p, J=9.7, 9.1 Hz, 3H), 1.59 (s, 1H), 1.57-1.47 (m, 2H), 1.36 (s, 8H), 1.25 (d, J=2.7 Hz, 1H), 1.20-1.14 (m, 1H), 0.88 (dt, J=13.3, 6.8 Hz, 7H). ESI-MS m/z calc. 707.33527, found 708.4 (M+1)⁺; Retention time: 1.48 minutes (LC method J).

### Step 3: tert-Butyl N-[2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]spiro[3.3]heptan-6-yl]carbamate, diastereomer 1 and 2

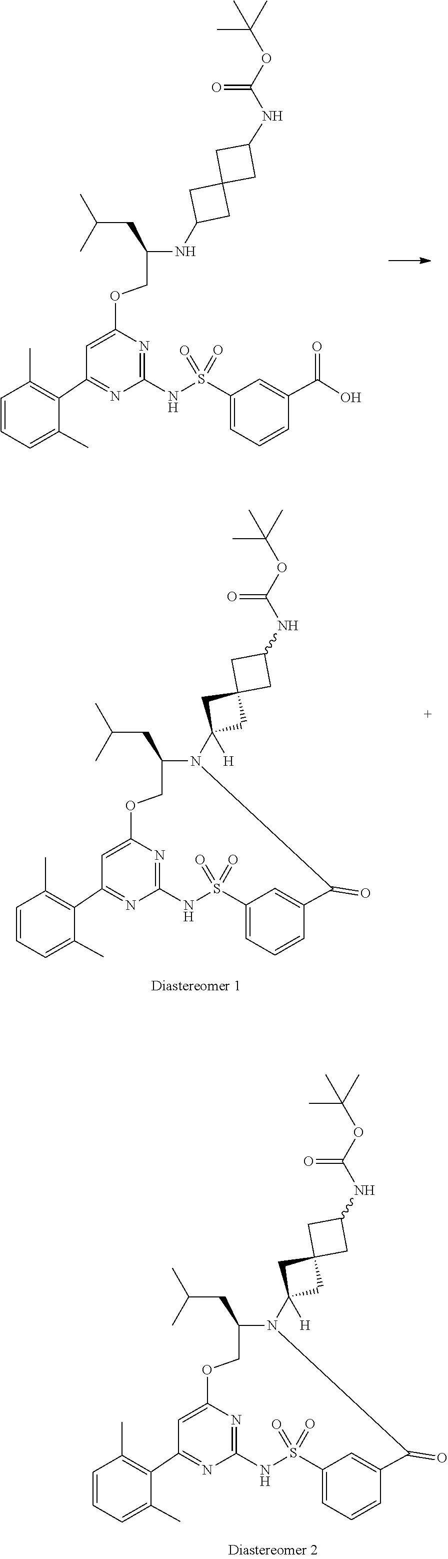

3-[[4-[(2R)-2-[[2-(tert-Butoxycarbonylamino)spiro[3.3]heptan-6-yl]amino]-4-methyl-pentoxy]-6-(2,6-dimethylphenyl)pyrimidin-2-yl]sulfamoyl]benzoic acid (hydrochloride salt)(4.3 g, 5.777 mmol) was dissolved in DMF (35 mL) and slowly added to a stirring solution of HATU (2.639 g, 6.941 mmol) and triethylamine (2.34 g, 23.12 mmol) in DMF (35 mL). The reaction mixture was stirred at room temperature overnight. An additional amount of HATU (1.32 g, 3.472 mmol) and triethylamine (1.17 g, 11.56 mmol) in DMF (12 mL) was added. Stirring was continued for another 4 hours then the reaction was partitioned between ethyl acetate and 1M HCl solution. The organics were separated, washed with brine, dried over sodium sulfate and evaporated to dryness. The crude material was purified by silica gel chromatography eluting with 30-100% ethyl acetate in hexanes. The product tert-butyl N-[2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]spiro[3.3]heptan-6-yl]carbamate (2.1742 g, 44%) ESI-MS m/z calc. 689.3247, found 690.3 (M+1)⁺; Retention time: 1.13 minutes, was subjected to SFC separation using a Phenomenex LUX-4 (250×21.2 mm), 5 m column at 40° C. and a mobile phase comprised of 24% of MeOH (no modifier) and 76% of CO₂, at flow rate of 70 mL/min. Two products were isolated (>98% ee each): diastereomer 1, peak 1, tert-butyl N-[2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (773.3 mg, 71%)¹H NMR (400 MHz, Methanol-d4) δ 8.41 (s, 1H), 7.90 (d, J=7.6 Hz, 1H), 7.61 (d, J=7.6 Hz, 1H), 7.56 (t, J=7.6 Hz, 1H), 7.17 (t, J 7.6 Hz, 1H), 7.04 (d, J=7.6 Hz, 2H), 6.15 (s, 1H), 5.20 (dd, J=10.8, 4.1 Hz, 1H), 4.17 (t, J=11.1 Hz, 1H), 3.82 (p, J=8.6 Hz, 2H), 3.72 (td, J=10.8, 5.3 Hz, 1H), 2.99 (dt, J=20.6, 9.9 Hz, 2H), 2.40 (dt, J=11.7, 6.3 Hz, 1H), 2.30 (h, J=9.1, 8.2 Hz, 2H), 2.17-2.07 (m, 2H), 1.99 (s, 2H), 1.90 (q, J=8.6 Hz, 4H), 1.61 (ddd, J=13.9, 10.7, 2.7 Hz, 1H), 1.33 (s, 9H), 1.29 (s, 1H), 1.17 (ddd, J=13.4, 7.8, 2.6 Hz, 2H), 0.72 (d, J=6.6 Hz, 3H), 0.23 (d, J=6.4 Hz, 3H). ESI-MS m/z calc. 689.3247, found 690.4 (M+1)⁺; Retention time: 2.05 minutes (LC method A); and diastereomer 2, peak 2, tert-butyl N-[2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate (726 mg, 67%), ¹H NMR (400 MHz, Methanol-d₄) δ 8.35 (s, 1H), 7.82 (d, J=7.5 Hz, 1H), 7.53 (d, J=7.5 Hz, 1H), 7.48 (t, J=7.6 Hz, 1H), 7.09 (t, J=7.6 Hz, 1H), 6.96 (d, J=7.6 Hz, 2H), 6.07 (s, 1H), 5.14 (dd, J=10.8, 4.1 Hz, 1H), 4.11 (t, J=11.1 Hz, 1H), 3.74 (p, J=8.4 Hz, 2H), 3.64 (t, J=11.4 Hz, 1H), 3.13 (s, 2H), 2.91 (d, J=9.8 Hz, 1H), 2.86 (d, J=10.1 Hz, 1H), 2.35 (dt, J=11.8, 6.1 Hz, 1H), 2.27-2.17 (m, 2H), 2.03 (d, J=8.1 Hz, 1H), 1.91 (s, 3H), 1.85 (d, J=9.8 Hz, 3H), 1.80 (d, J=9.5 Hz, 1H), 1.52 (t, J=12.6 Hz, 1H), 1.25 (s, 9H), 1.23-1.16 (m, 1H), 1.10 (d, J=11.0 Hz, 2H), 0.63 (d, J=6.4 Hz, 3H), 0.15 (d, J=6.2 Hz, 3H). ESI-MS m/z calc. 689.3247, found 690.4 (M+1)⁺; Retention time: 2.04 minutes (LC method A).

### Step 4: (11R)-12-{6-Aminospiro[3.3]heptan-2-yl}-6-(2,6-dimethylphenyl)-11-(2-methylpropyl)-9-oxa-2λ⁶-thia-3,5,12,19-tetraazatricyclo[12.3.1.14,8]nonadeca-1(18),4,6,8(19),14,16-hexaene-2,2,13-trione, diastereomer 1

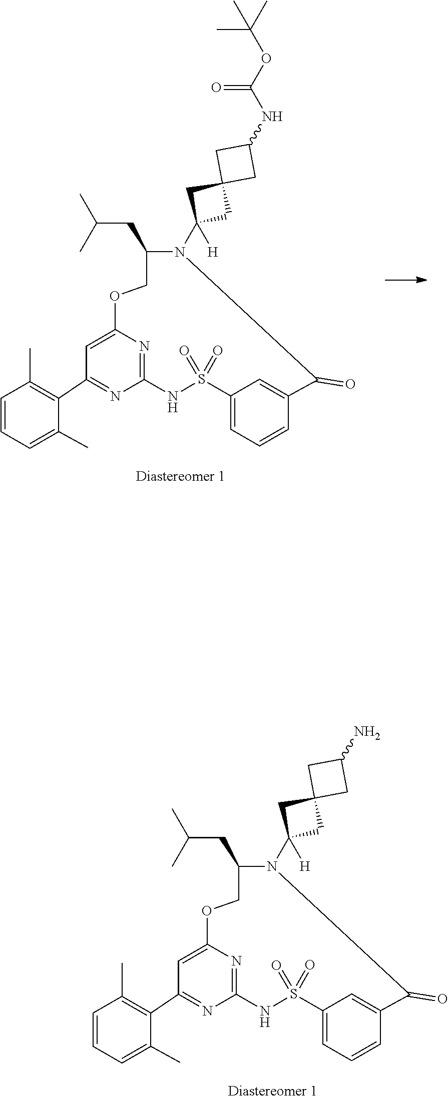

tert-Butyl N-[2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4,6,8(19),14,16-hexaen-12-yl]spiro[3.3]heptan-6-yl]carbamate, diastereomer 1 (736 mg, 1.067 mmol) was dissolved in DCM (4 mL) and HCl (4 mL of 4 M, 16.00 mmol) was added. The reaction was stirred at room temperature for 1 hour, then the solvent was evaporated. Hexanes were added and the evaporated twice to give an off-white solid (11R)-12-{6-aminospiro[3.3]heptan-2-yl}-6-(2,6-dimethylphenyl)-11-(2-methylpropyl)-9-oxa-2λ⁶-thia-3,5,12,19-tetraazatricyclo[12.3.1.14,8]nonadeca-1(18),4,6,8(19),14,16-hexaene-2,2,13-trione, diastereomer 1 (hydrochloride salt)(764 mg, 114%) ESI-MS m/z calc. 589.2723, found 590.5 (M+1)⁺; Retention time: 0.52 minutes (LC method D).

### Step 5: Propan-2-yl N-{6-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(18),4,6,8(19),14,16-hexaen-12-yl]spiro[3.3]heptan-2-yl}carbamate (Compound 123)

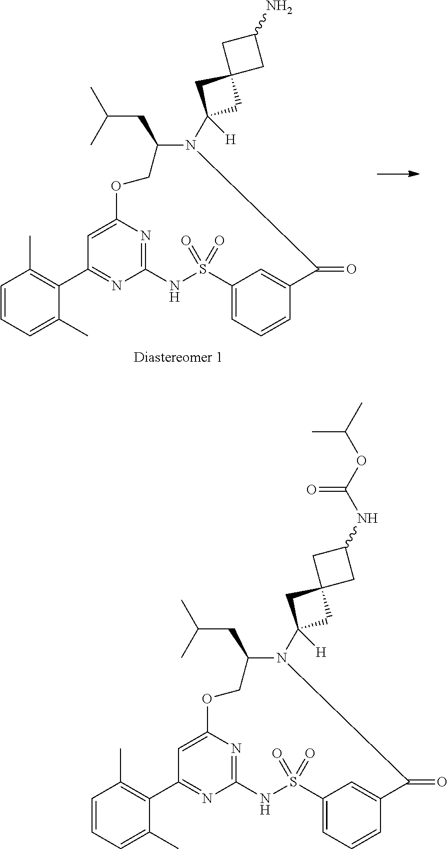

(11R)-12-{6-Aminospiro[3.3]heptan-2-yl}-6-(2,6-dimethylphenyl)-11-(2-methylpropyl)-9-oxa-2λ⁶-thia-3,5,12,19-tetraazatricyclo[12.3.1.14,8]nonadeca-1(18),4,6,8(19),14,16-hexaene-2,2,13-trione (hydrochloride salt)(diastereomer 1, 10.5 mg, 0.01677 mmol) was combined in DCM with isopropyl chloroformate (approximately 16.77 μL of 2 M, 0.03354 mmol) and DIPEA (approximately 10.84 mg, 14.61 μL, 0.08385 mmol), and the reaction was stirred at room temperature for 30 minutes. After this time the reaction mixture was quenched with several drops of aqueous 1M HCl. The reaction mixture was then partially concentrated, diluted with 1:1 DMSO and methanol, filtered, and purified by preparative HPLC (1-99% ACN in water, HCl modifier, 15 minute run) to give upon drying propan-2-yl N-{6-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(18),4,6,8(19),14,16-hexaen-12-yl]spiro[3.3]heptan-2-yl}carbamate (7.5 mg, 66%). ESI-MS m/z calc. 675.3091, found 676.7 (M+1)⁺; Retention time: 1.95 minutes; (LC method A).

### Example 99: Preparation of Compound 124

### Step 1: Propan-2-yl N-{6-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(18),4,6,8(19),14,16-hexaen-12-yl]spiro[3.3]heptan-2-yl}carbamate (Compound 124)

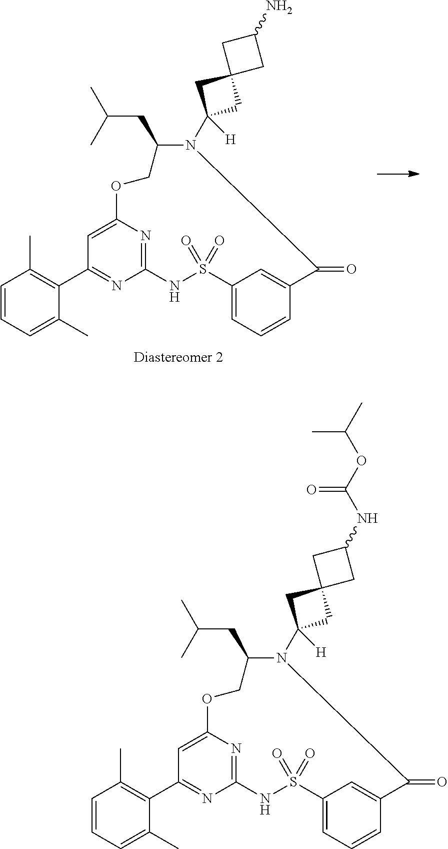

(11R)-12-(6-Aminospiro[3.3]heptan-2-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one (hydrochloride salt), diastereomer 2, prepared in a manner analogous to that described above (10 mg, 0.01597 mmol) was combined in DCM with isopropyl chloroformate (approximately 15.97 μL of 2 M, 0.03194 mmol) and DIPEA (approximately 10.32 mg, 13.91 μL, 0.07985 mmol), and the reaction was stirred at room temperature for 30 minutes. After this time the reaction mixture was quenched with several drops of aqueous 1M HCl. The reaction mixture was then partially concentrated, diluted with 1:1 DMSO and methanol, filtered, and purified by preparative HPLC (1-99% ACN in water, HCL modifier, 15 minute run) to give upon drying propan-2-yl N-{6-[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(18),4,6,8(19),14,16-hexaen-12-yl]spiro[3.3]heptan-2-yl}carbamate (7 mg, 64%). ESI-MS m/z calc. 675.3091, found 676.6 (M+1)⁺; Retention time: 1.94 minutes; (LC method A).

### Example 100: Preparation of Compound 109 and Compound 125

### Step 1: 3-[[4-[2-(tert-Butoxycarbonylamino)-4,4,4-trifluoro-butoxy]-6-(2,6-dimethylphenyl)pyrimidin-2-yl]sulfamoyl]benzoic acid

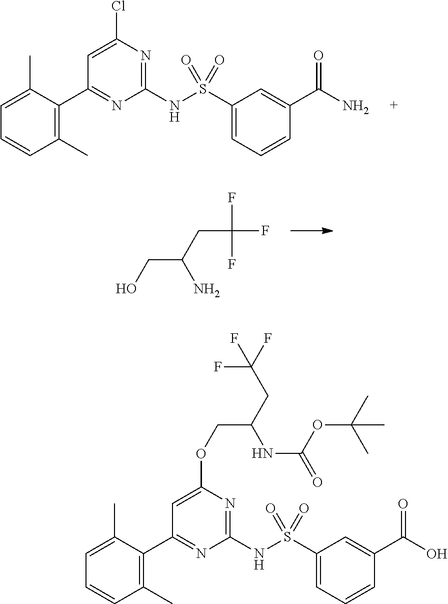 [SMILES: Cc1cccc(C)c1-c1cc(Cl)nc(NS(=O)(=O)c2cccc(C(N)=O)c2)n1 ; NC(CO)CC(F)(F)F ; Cc1cccc(C)c1-c1cc(OCC(CC(F)(F)F)NC(=O)OC(C)(C)C)nc(NS(=O)(=O)c2cccc(C(=O)O)c2)n1]

A solution of 3-[[4-chloro-6-(2,6-dimethylphenyl)pyrimidin-2-yl]sulfamoyl]benzoic acid (0.63 g, 1.508 mmol), 2-amino-4,4,4-trifluoro-butan-1-ol (hydrochloride salt) (0.54 g, 3.007 mmol), and sodium t-butoxide (0.73 g, 7.596 mmol) in THF (8 mL) was stirred for five minutes, turning bright yellow. The reaction was placed in a preheated 60° C. bath and stirred for 25 minutes. UPLCMS showed complete conversion to amino intermediate. After cooling to room temperature, di-tert-butyl dicarbonate (0.67 g, 3.070 mmol) was added, and the reaction was stirred for 17 hours. The reaction was quenched with 1 M hydrochloric acid, diluted with water, and extracted with ethyl acetate. The combined extracts were washed with water, dried over sodium sulfate, and evaporated under vacuum. The residue was purified by silica gel column chromatography with 0-10% methanol in dichloromethane to give a mixture containing product. The mixture was re-purified by silica gel column chromatography with 0-9% methanol in dichloromethane to give 3-[[4-[2-(tert-butoxycarbonylamino)-4,4,4-trifluoro-butoxy]-6-(2,6-dimethylphenyl)pyrimidin-2-yl]sulfamoyl]benzoic acid (0.54 g, 57%) ESI-MS m/z calc. 624.1866, found 625.3 (M+1)⁺; Retention time: 0.67 minutes as a colorless solid, LC method D.

### Step 2: 3-[[4-(2-Amino-4,4,4-trifluoro-butoxy)-6-(2,6-dimethylphenyl)pyrimidin-2-yl]sulfamoyl]benzoic acid

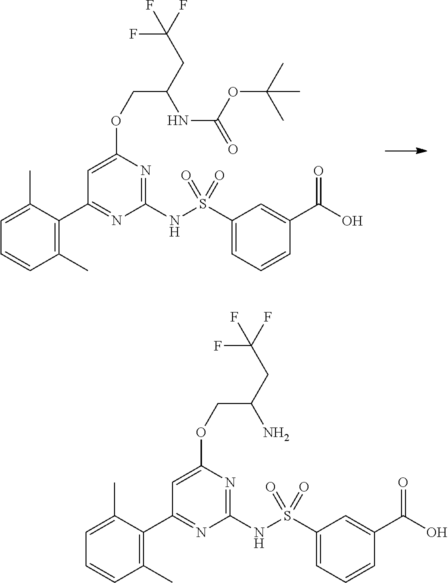 [SMILES: Cc1cccc(C)c1-c1cc(OCC(CC(F)(F)F)NC(=O)OC(C)(C)C)nc(NS(=O)(=O)c2cccc(C(=O)O)c2)n1 ; Cc1cccc(C)c1-c1cc(OCC(N)CC(F)(F)F)nc(NS(=O)(=O)c2cccc(C(=O)O)c2)n1]

A solution of 3-[[4-[2-(tert-butoxycarbonylamino)-4,4,4-trifluoro-butoxy]-6-(2,6-dimethylphenyl)pyrimidin-2-yl]sulfamoyl]benzoic acid (83 mg, 0.1329 mmol) and HCl (4 mL of 4 M, 16.00 mmol) (in dioxane) was stirred for one hour. The solvent was removed under vacuum, and the solids were triturated with diethyl ether to give 3-[[4-(2-amino-4,4,4-trifluoro-butoxy)-6-(2,6-dimethylphenyl)pyrimidin-2-yl]sulfamoyl]benzoic acid (hydrochloride salt) (81 mg, 109%) ESI-MS m/z calc. 524.13416, found 525.2 (M+1)⁺; Retention time: 0.39 minutes as a colorless solid, LC method D.

### Step 3: 3-[[4-(2,6-Dimethylphenyl)-6-[4,4,4-trifluoro-2-(spiro[2.3]hexan-5-ylamino)butoxy]pyrimidin-2-yl]sulfamoyl]benzoic acid

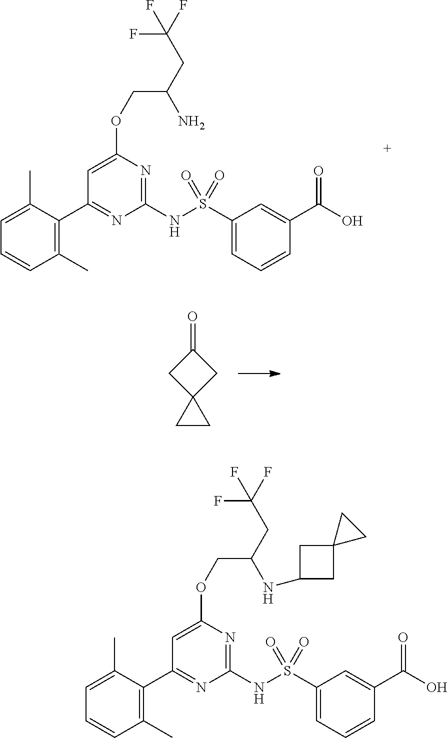 [SMILES: Cc1cccc(C)c1-c1cc(OCC(N)CC(F)(F)F)nc(NS(=O)(=O)c2cccc(C(=O)O)c2)n1 ; O=C1CC2(CC2)C1 ; Cc1cccc(C)c1-c1cc(OCC(CC(F)(F)F)NC2CC3(CC3)C2)nc(NS(=O)(=O)c2cccc(C(=O)O)c2)n1]

A solution of 3-[[4-(2-amino-4,4,4-trifluoro-butoxy)-6-(2,6-dimethylphenyl)pyrimidin-2-yl]sulfamoyl]benzoic acid (hydrochloride salt) (0.14 g, 0.2496 mmol) and spiro[2.3]hexan-5-one (0.12 g, 1.248 mmol) in dichloroethane (1 mL) and acetic acid (0.2 μL) was stirred for 15 minutes, and sodium cyanoborohydride (48 mg, 0.7638 mmol) was added. The reaction was stirred for 6 hours, more spiro[2.3]hexan-5-one (0.12 mL, 1.248 mmol) and sodium cyanoborohydride (49 mg, 0.7797 mmol) were added, and the reaction was stirred for 16 hours. The reaction was quenched with 1 M hydrochloric acid, and the solvents were evaporated. The residue was purified by reverse-phase HPLC-MS (1%-99% acetonitrile/water (5 mM HCl)) to give 3-[[4-(2,6-dimethylphenyl)-6-[4,4,4-trifluoro-2-(spiro[2.3]hexan-5-ylamino)butoxy]pyrimidin-2-yl]sulfamoyl]benzoic acid (hydrochloride salt) (82 mg, 51%) ESI-MS m/z calc. 604.1967, found 605.3 (M+1)⁺; Retention time: 0.84 minutes as a colorless solid, LC method D.

### Step 4: 6-(2,6-dimethylphenyl)-12-{spiro[2.3]hexan-5-yl}-11-(2,2,2-trifluoroethyl)-9-oxa-2λ⁶-thia-3,5,12,19-tetraazatricyclo[12.3.1.14,8]nonadeca-1(17),4(19),5,7,14(18),15-hexaene-2,2,13-trione

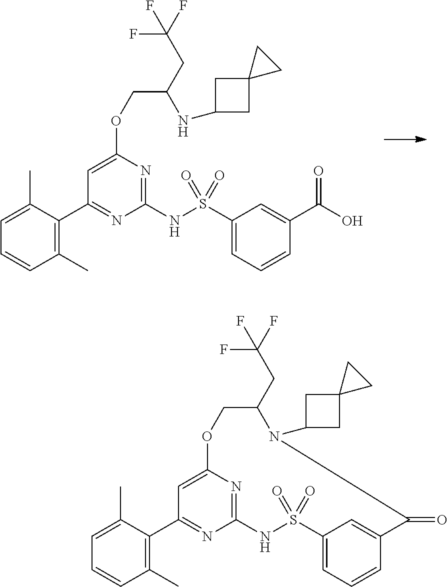 [SMILES: Cc1cccc(C)c1-c1cc(OCC(CC(F)(F)F)NC2CC3(CC3)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CC3)C1)C(CC(F)(F)F)CO2]

A solution of 3-[[4-(2,6-dimethylphenyl)-6-[4,4,4-trifluoro-2-(spiro[2.3]hexan-5-ylamino)butoxy]pyrimidin-2-yl]sulfamoyl]benzoic acid (hydrochloride salt) (82 mg, 0.1279 mmol), HATU (74 mg, 0.1946 mmol), and triethylamine (72 μL, 0.5166 mmol) in DMF (7 mL) was stirred for 18 hours. The reaction was acidified with 1 M citric acid, diluted with water, and extracted with ethyl acetate. The combined extracts were washed with brine, dried over sodium sulfate, and evaporated under vacuum. The residue was purified by reverse-phase HPLC-MS (1%-99% acetonitrile/water (5 mM HCl)) to give 6-(2,6-dimethylphenyl)-12-{spiro[2.3]hexan-5-yl}-11-(2,2,2-trifluoroethyl)-9-oxa-2λ⁶-thia-3,5,12,19-tetraazatricyclo[12.3.1.14,8]nonadeca-1(17),4(19),5,7,14(18),15-hexaene-2,2,13-trione (11 mg, 15%) ESI-MS m/z calc. 586.18616, found 587.3 (M+1)⁺; Retention time: 0.73 minutes as a colorless solid, LC method D.

### Step 5: 6-(2,6-Dimethylphenyl)-2,2-dioxo-12-spiro[2.3]hexan-5-yl-11-(2,2,2-trifluoroethyl)-9-oxa-2λ⁶-thia-3,5,12,19-tetrazatricyclo[12.3.1.14,8]nonadeca-1(18),4(19),5,7,14,16-hexaen-13-one, enantiomer 1, (Compound 109), and 6-(2,6-dimethylphenyl)-12-{spiro[2.3]hexan-5-yl}-11-(2,2,2-trifluoroethyl)-9-oxa-2λ⁶-thia-3,5,12,19-tetraazatricyclo[12.3.1.14,8]nonadeca-1(17),4(19),5,7,14(18),15-hexaene-2,2,13-trione, enantiomer 2 (Compound 125)

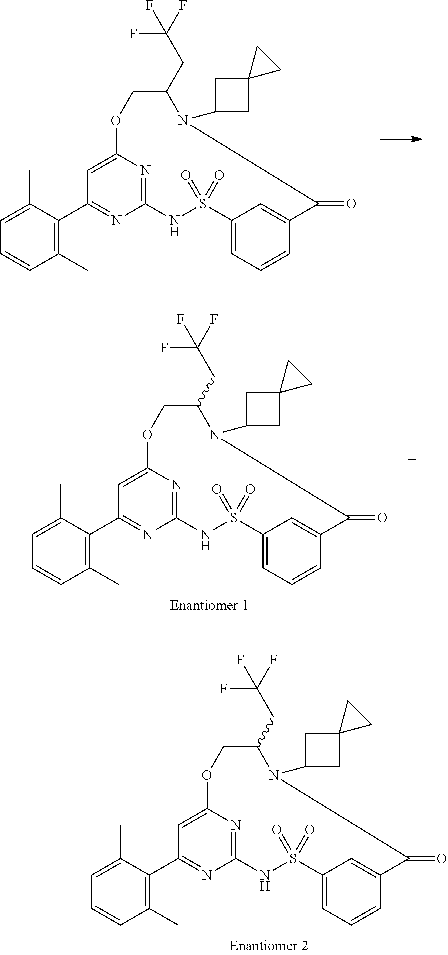 [SMILES: Cc1cccc(C)c1-c1cc2nc(n1)NS(=O)(=O)c1cccc(c1)C(=O)N(C1CC3(CC3)C1)C(CC(F)(F)F)CO2 ; Cc1cccc(C)c1-c1cc2nc(n1)NS(=O)(=O)c1cccc(c1)C(=O)N(C1CC3(CC3)C1)C(CC(F)(F)F)CO2 ; Cc1cccc(C)c1-c1cc2nc(n1)NS(=O)(=O)c1cccc(c1)C(=O)N(C1CC3(CC3)C1)C(CC(F)(F)F)CO2]

6-(2,6-Dimethylphenyl)-12-{spiro[2.3]hexan-5-yl}-11-(2,2,2-trifluoroethyl)-9-oxa-2λ⁶-thia-3,5,12,19-tetraazatricyclo[12.3.1.14,8]nonadeca-1(17),4(19),5,7,14(18),15-hexaene-2,2,13-trione (16 mg, 0.02727 mmol) was subjected to normal phase SFC using a (R,R)-Whelk-O column (150×2.1 mm, 3.5 m particle size) sold by Regis Technologies (pn: 780230), and a gradient run from 5-80% mobile phase B over 17.5 minutes. Mobile phase A=CO₂. Mobile phase B=MeOH (20 mM NH3). Flow rate=40 mL/min [20 mM NH₃], and column temperature=55° C. Two enantiomers were isolated: enantiomer 1,6-(2,6-dimethylphenyl)-2,2-dioxo-12-spiro[2.3]hexan-5-yl-11-(2,2,2-trifluoroethyl)-9-oxa-2λ⁶-thia-3,5,12,19-tetrazatricyclo[12.3.1.14,8]nonadeca-1(18),4(19),5,7,14,16-hexaen-13-one (4.7 mg, 58%) ESI-MS m/z calc. 586.18616, found 587.3 (M+1)⁺; Retention time: 1.82 minutes (LC method A); enantiomer 2,6-(2,6-dimethylphenyl)-12-{spiro[2.3]hexan-5-yl}-11-(2,2,2-trifluoroethyl)-9-oxa-2λ⁶-thia-3,5,12,19-tetraazatricyclo[12.3.1.14,8]nonadeca-1(17),4(19),5,7,14(18),15-hexaene-2,2,13-trione (3.5 mg, 43%) ESI-MS m/z calc. 586.18616, found 587.3 (M+1)⁺; Retention time: 1.82 minutes (LC method A), both obtained as colorless solids.

### Example 101: Preparation of Compound 126

### Step 1: 3-[[4-[(2R)-2-(tert-Butoxycarbonylamino)propoxy]-6-(2,6-dimethylphenyl)pyrimidin-2-yl]sulfamoyl]benzoic acid

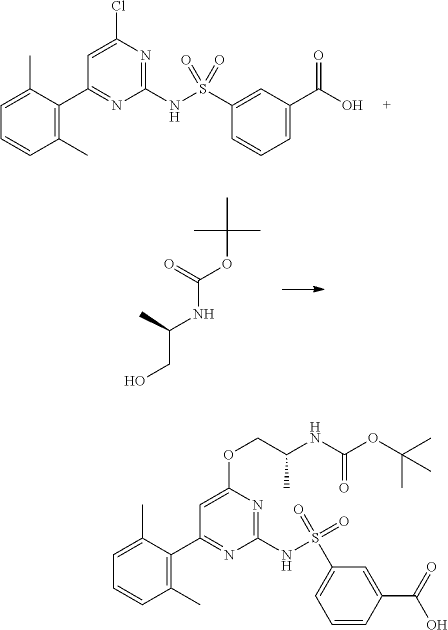 [SMILES: Cc1cccc(C)c1-c1cc(Cl)nc(NS(=O)(=O)c2cccc(C(=O)O)c2)n1 ; C[C@H](CO)NC(=O)OC(C)(C)C ; Cc1cccc(C)c1-c1cc(OC[C@@H](C)NC(=O)OC(C)(C)C)nc(NS(=O)(=O)c2cccc(C(=O)O)c2)n1]

A solution of 3-[[4-chloro-6-(2,6-dimethylphenyl)pyrimidin-2-yl]sulfamoyl]benzoic acid (75 mg, 0.1795 mmol) in THE (0.7 mL) was added to tert-butyl N-[(1R)-2-hydroxy-1-methyl-ethyl]carbamate (approximately 47.17 mg, 0.2692 mmol). Solid sodium tert-butoxide (approximately 86.25 mg, 0.8975 mmol) was added after. The reaction mixture was allowed to stir overnight at room temperature. acetic acid (approximately 64.68 mg, 61.25 μL, 1.077 mmol) was added. The reaction mixture was diluted with DCM and washed with HCl (1 M, 1×7 mL) and brine (2×75 mL). The organic layer was dried over sodium sulfate, filtered and concentrated under reduced pressure. The crude product was chromatographed on a 12 gram silica gel column eluting with a EtOAc/hexane gradient. 3-[[4-[(2R)-2-(tert-Butoxycarbonylamino)propoxy]-6-(2,6-dimethylphenyl)pyrimidin-2-yl]sulfamoyl]benzoic acid (1.65 g, 2.964 mmol) (65 mg, 65%) was obtained. ESI-MS m/z calc. 556.19916, found 557.3 (M+1)⁺; Retention time: 1.63 minutes; LC method A.

### Step 2: 3-[[4-[(2R)-2-Aminopropoxy]-6-(2,6-dimethylphenyl)pyrimidin-2-yl]sulfamoyl]benzoic acid

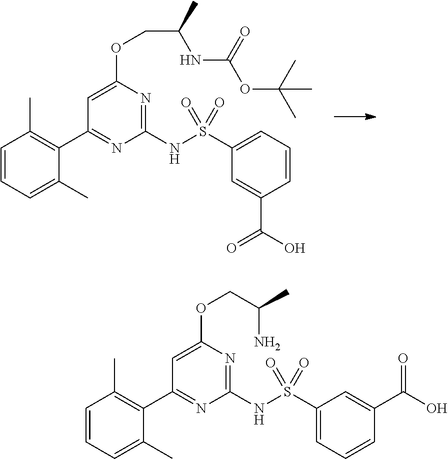

A solution of 3-[[4-[(2R)-2-(tert-butoxycarbonylamino)propoxy]-6-(2,6-dimethylphenyl)pyrimidin-2-yl]sulfamoyl]benzoic acid (1.65 g, 2.964 mmol) in HCl (8 mL of 4 M, 32.00 mmol) (in dioxane) was stirred for two hours, and the solvent was removed under vacuum. The solids were triturated with diethyl ether and dried under vacuum to give 3-[[4-[(2R)-2-aminopropoxy]-6-(2,6-dimethylphenyl)pyrimidin-2-yl]sulfamoyl]benzoic acid (hydrochloride salt) (1.55 g, 106%) as a colorless solid. ESI-MS m/z calc. 456.14673, found 457.2 (M+1)⁺; Retention time: 0.37 minutes, LC method D.

### Step 3: 3-[[4-(2,6-Dimethylphenyl)-6-[(2R)-2-(spiro[2.3]hexan-5-ylamino)propoxy]pyrimidin-2-yl]sulfamoyl]benzoic acid

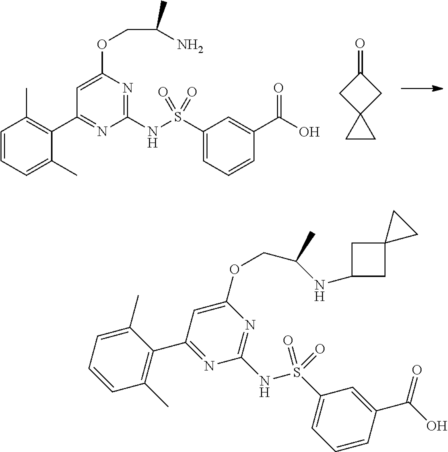

A mixture of 3-[[4-[(2R)-2-aminopropoxy]-6-(2,6-dimethylphenyl)pyrimidin-2-yl]sulfamoyl]benzoic acid (hydrochloride salt) (49.30 mg, 0.1 mmol) and spiro[2.3]hexan-5-one (approximately 48.06 mg, 0.5000 mmol) in dichloroethane (0.5 mL) and acetic acid (0.1 mL) was stirred for 15 minutes, and sodium cyanoborohydride (approximately 18.85 mg, 0.3000 mmol) was added. After 16 hours, more spiro[2.3]hexan-5-one (approximately 48.06 mg, 0.5000 mmol) and sodium cyanoborohydride (approximately 18.85 mg, 0.3000 mmol) were added again. After four hours, the reaction was diluted with methanol and purified by reverse-phase HPLC-MS (1%-99% acetonitrile/water (5 mM HCl)) to give 3-[[4-(2,6-dimethylphenyl)-6-[(2R)-2-(spiro[2.3]hexan-5-ylamino)propoxy]pyrimidin-2-yl]sulfamoyl]benzoic acid (hydrochloride salt) (36 mg, 63%) as colorless solid. ESI-MS m z calc. 536.20935, found 537.3 (M+1)⁺; Retention time: 0.41 minutes; LC method D.

### Step 4: (11R)-6-(2,6-dimethylphenyl)-11-methyl-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione (Compound 126)

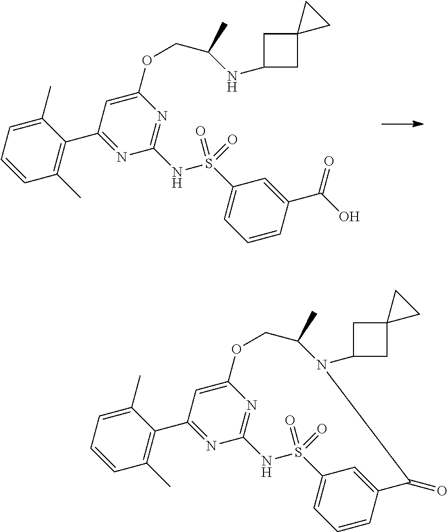 [SMILES: Cc1cccc(C)c1-c1cc(OC[C@@H](C)NC2CC3(CC3)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CC3)C1)[C@H](C)CO2]

A solution of 3-[[4-(2,6-dimethylphenyl)-6-[(2R)-2-(spiro[2.3]hexan-5-ylamino)propoxy]pyrimidin-2-yl]sulfamoyl]benzoic acid (hydrochloride salt) (36 mg, 0.06282 mmol), HATU (38 mg, 0.09994 mmol), and triethylamine (36 μL, 0.2583 mmol) in DMF (3 mL) was stirred for four days. The reaction was concentrated and purified by reverse-phase HPLC-MS (1%-99% acetonitrile/water (5 mM HCl)) to give (11R)-6-(2,6-dimethylphenyl)-11-methyl-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione as a tan solid (15.5 mg, 47%). ESI-MS m/z calc. 518.1988, found 519.3 (M+1)⁺; Retention time: 1.77 minutes, LC method A.

### Example 102: Preparation of Compound 127

### Step 1: 3-[[4-[(2R)-2-[(7-tert-Butoxycarbonyl-7-azaspiro[3.5]nonan-2-yl)amino]-3-[1-(trifluoromethyl)cyclopropyl]propoxy]-6-(2,6-dimethylphenyl)pyrimidin-2-yl]sulfamoyl]benzoic acid

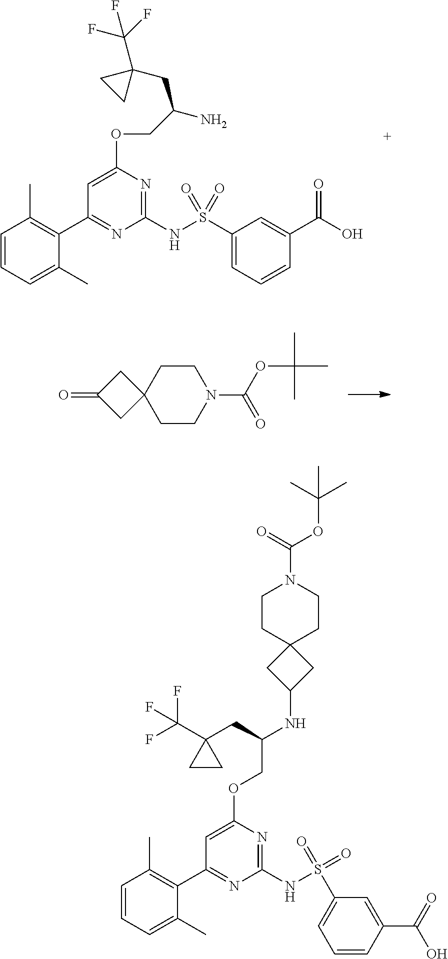 [SMILES: Cc1cccc(C)c1-c1cc(OC[C@H](N)CC2(C(F)(F)F)CC2)nc(NS(=O)(=O)c2cccc(C(=O)O)c2)n1 ; CC(C)(C)OC(=O)N1CCC2(CC1)CC(=O)C2 ; Cc1cccc(C)c1-c1cc(OC[C@@H](CC2(C(F)(F)F)CC2)NC2CC3(CCN(C(=O)OC(C)(C)C)CC3)C2)nc(NS(=O)(=O)c2cccc(C(=O)O)c2)n1]

3-[[4-[(2R)-2-amino-3-[1-(trifluoromethyl)cyclopropyl]propoxy]-6-(2,6-dimethylphenyl)pyrimidin-2-yl]sulfamoyl]benzoic acid (400 mg, 0.5668 mmol) was dissolved in DCM (4 mL) then tert-butyl 2-oxo-7-azaspiro[3.5]nonane-7-carboxylate (153 mg, 0.6393 mmol) and Sodium triacetoxyborohydride (178 mg, 0.8399 mmol) were added. The reaction was stirred at room temperature for 30 minutes then quenched by the addition of 2M HCl (4 mL). The layers were separated, and the aqueous layer extracted three times with DCM (4 mL). The organic layer was dried over sodium sulfate and concentrated. The crude residue was dry loaded on to silica gel and purified by flash column chromatography using 0-10%0 Methanol in DCM. The appropriate fractions were collected to give 3-[[4-[(2R)-2-[(7-tert-butoxycarbonyl-7-azaspiro[3.5]nonan-2-yl)amino]-3-[1-(trifluoromethyl)cyclopropyl]propoxy]-6-(2,6-dimethylphenyl)pyrimidin-2-yl]sulfamoyl]benzoic acid (hydrochloride salt) (406.3 mg, 700%) as a white solid. ESI-MS m/z calc. 787.3227, found 788.6 (M+1)⁺; Retention time: 2.9 minutes.

### Step 2: tert-Butyl 2-1(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (Compound 127)

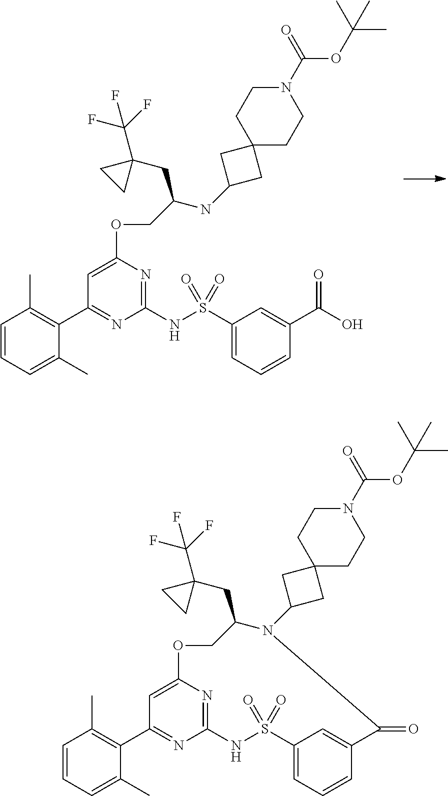 [SMILES: Cc1cccc(C)c1-c1cc(OC[C@@H](CC2(C(F)(F)F)CC2)NC2CC3(CCN(C(=O)OC(C)(C)C)CC3)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CCN(C(=O)OC(C)(C)C)CC3)C1)[C@H](CC1(C(F)(F)F)CC1)CO2]

HATU (1.56 g, 4.1028 mmol) and DIPEA (2.0034 g, 2.70 mL, 15.501 mmol) were dissolved in DMF (24 mL) then 3-[[4-[(2R)-2-[(7-tert-butoxycarbonyl-7-azaspiro[3.5]nonan-2-yl)amino]-3-[1-(trifluoromethyl)cyclopropyl]propoxy]-6-(2,6-dimethylphenyl)pyrimidin-2-yl]sulfamoyl]benzoic acid (2.47 g, 3.1350 mmol) in DMF (24 mL) was added drop wise. The solution was stirred overnight. The reaction was quenched by the addition of brine (200 mL). The aqueous layer was extracted three times with EtOAc (40 mL each). The organic layer was washed 5 times with brine (25 mL each). Then dried over sodium sulfate and concentrated. The crude residue was dry loaded on to silica gel and purified by flash column chromatography using 0-20% acetone in DCM to give tert-butyl 2-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (1.49 g, 59%) as an off white powder. ESI-MS m/z calc. 769.3121, found 770.9 (M+1)⁺; Retention time: 3.85 minutes (LC method T).

### Example 103: Preparation of Compound 128

### Step 1: (11R)-12-(7-Azaspiro[3.5]nonan-2-yl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one

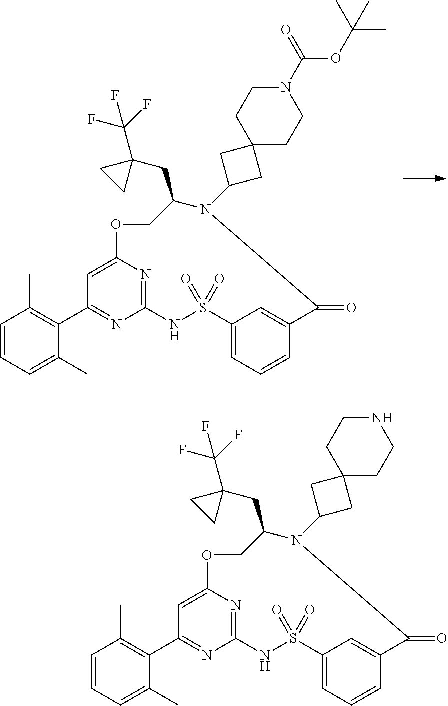 [SMILES: Cc1cccc(C)c1-c1cc2nc(n1)NS(=O)(=O)c1cccc(c1)C(=O)N(C1CC3(CCN(C(=O)OC(C)(C)C)CC3)C1)[C@H](CC1(C(F)(F)F)CC1)CO2 ; Cc1cccc(C)c1-c1cc2nc(n1)NS(=O)(=O)c1cccc(c1)C(=O)N(C1CC3(CCNCC3)C1)[C@H](CC1(C(F)(F)F)CC1)CO2]

tert-Butyl 2-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (21.4 mg, 0.02682 mmol) was dissolved in 4M HCl in dioxane (1 mL of 4 M, 4.000 mmol) and stirred at room temperature. After 1 h, the reaction was evaporated to dryness to give (11R)-12-(7-azaspiro[3.5]nonan-2-yl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (hydrochloride salt) (18.9 mg, 100%) ESI-MS m/z calc. 669.25964, found 670.7 (M+1)⁺; Retention time: 0.5 minutes. LC method D.

### Step 2: (11R)-6-(2,6-Dimethylphenyl)-12-[7-(2-methoxyethyl)-7-azaspiro[3.5]nonan-2-yl]-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (Compound 128)

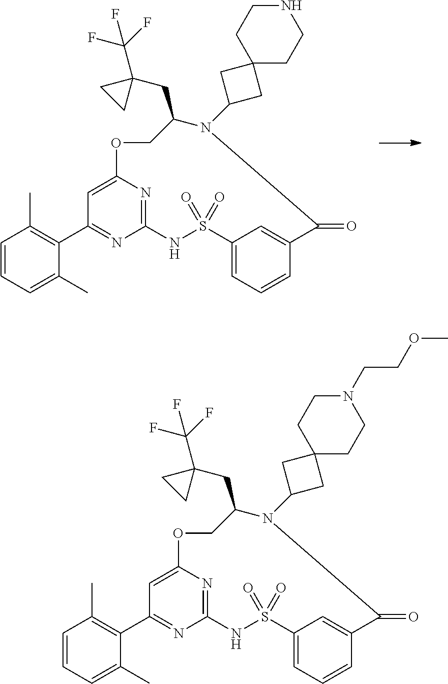 [SMILES: Cc1cccc(C)c1-c1cc2nc(n1)NS(=O)(=O)c1cccc(c1)C(=O)N(C1CC3(CCNCC3)C1)[C@H](CC1(C(F)(F)F)CC1)CO2 ; COCCN1CCC2(CC1)CC(N1C(=O)c3cccc(c3)S(=O)(=O)Nc3nc(cc(-c4c(C)cccc4C)n3)OC[C@H]1CC1(C(F)(F)F)CC1)C2]

A mixture of (11R)-12-(7-azaspiro[3.5]nonan-2-yl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (hydrochloride salt) (36.9 mg, 0.05225 mmol) and TEA (36 mg, 0.3558 mmol) in acetonitrile (0.4 mL) was heated to 55° C. and treated with 1-bromo-2-methoxy-ethane (6 μL, 0.06385 mmol). The reaction was stirred for 16 h. The solutions were filtered and the filtrate dissolved in 0.8 mL DMSO, and it was purified by reverse phase HPLC using a 15 min gradient of 1-99% MeCN in water (HCl modifier) to give (11R)-6-(2,6-dimethylphenyl)-12-[7-(2-methoxyethyl)-7-azaspiro[3.5]nonan-2-yl]-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (hydrochloride salt) (15.4 mg, 37%) ESI-MS m/z calc. 727.3015, found 728.7 (M+1)⁺; Retention time: 1.36 minutes, LC method A.

### Example 104: Preparation of Compound 129

### Step 1: (11R)-6-(2,6-dimethylphenyl)-12-[7-(2-isopropoxyethyl)-7-azaspiro[3.5]nonan-2-yl]-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (Compound 129)

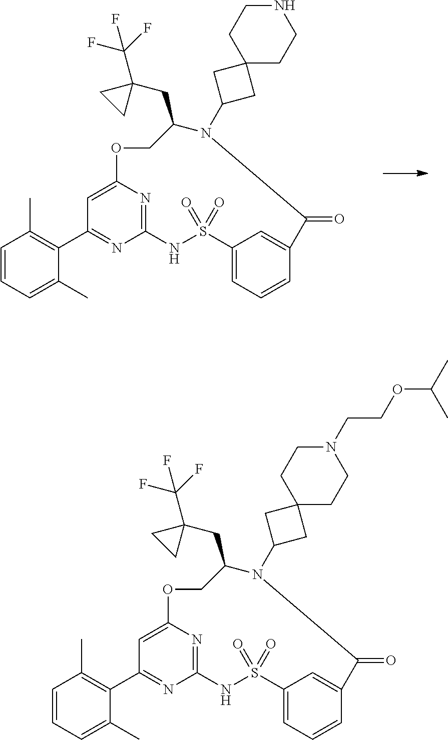 [SMILES: Cc1cccc(C)c1-c1cc2nc(n1)NS(=O)(=O)c1cccc(c1)C(=O)N(C1CC3(CCNCC3)C1)[C@H](CC1(C(F)(F)F)CC1)CO2 ; Cc1cccc(C)c1-c1cc2nc(n1)NS(=O)(=O)c1cccc(c1)C(=O)N(C1CC3(CCN(CCOC(C)C)CC3)C1)[C@H](CC1(C(F)(F)F)CC1)CO2]

A solution of 2-(2-bromoethoxy)propane (approximately 5.451 mg, 0.03263 mmol), (11R)-12-(7-azaspiro[3.5]nonan-2-yl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (hydrochloride salt) (19.2 mg, 0.02719 mmol), and TEA (approximately 19.26 mg, 26.53 μL, 0.1903 mmol) in acetonitrile (0.5 mL) was stirred at 50° C. for 8 h. The solution was filtered and the filtrate diluted with 0.8 mL MeOH/DMSO (1:1), and purified by reverse phase HPLC using a 15 min gradient of 1-99% MeCN in water (HCl modifier) to give (11R)-6-(2,6-dimethylphenyl)-12-[7-(2-isopropoxyethyl)-7-azaspiro[3.5]nonan-2-yl]-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (hydrochloride salt) (10.3 mg, 48%). ESI-MS m/z calc. 755.3328, found 756.8 (M+1)⁺; Retention time: 1.46 minutes; LC method A.

### Example 105: Preparation of Compound 130 and Compound 131

### Step 1: 3-[[4-(2,6-Dimethylphenyl)-6-[(2R)-2-[(3-isopropoxycyclobutyl)amino]-3-[1-(trifluoromethyl)cyclopropyl]propoxy]pyrimidin-2-yl]sulfamoyl]benzoic acid

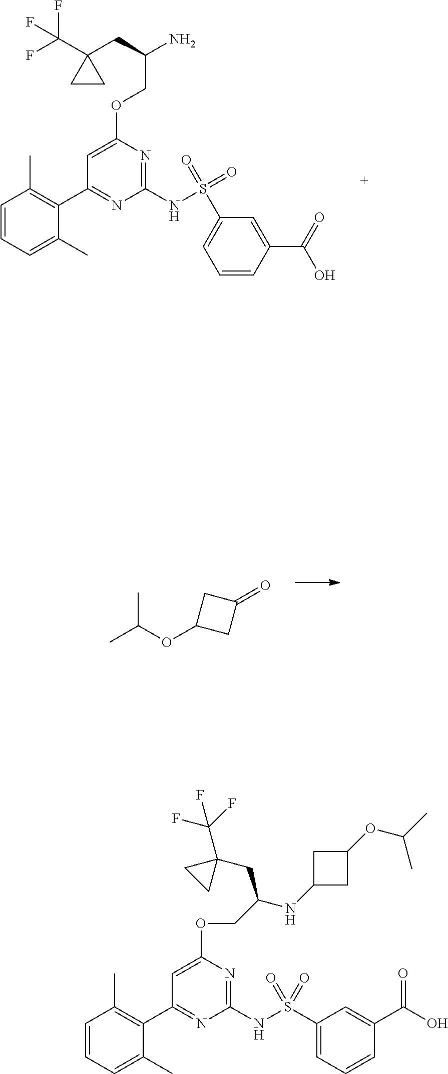

3-[[4-[(2R)-2-amino-3-[1-(trifluoromethyl)cyclopropyl]propoxy]-6-(2,6-dimethylphenyl)pyrimidin-2-yl]sulfamoyl]benzoic acid (hydrochloride salt) (100 mg, 0.1664 mmol), 3-isopropoxycyclobutanone (28.5 mg, 0.2224 mmol), and sodium triacetoxyborohydride (95.6 mg, 0.5060 mmol) were combined in DCM (0.3 mL) and stirred at room temperature for 2 h. The reaction was evaporated to dryness and the resulting material was purified by LC/MS utilizing a gradient of 1-99% acetonitrile in 5 mM aqueous HCl to yield 3-[[4-(2,6-dimethylphenyl)-6-[(2R)-2-[(3-isopropoxycyclobutyl)amino]-3-[1-(trifluoromethyl)cyclopropyl]propoxy]pyrimidin-2-yl]sulfamoyl]benzoic acid (hydrochloride salt) (62.8 mg, 53%) ESI-MS m/z calc. 676.2542, found 677.4 (M+1)⁺; Retention time: 0.53 minutes, LC method A.

### Step 2: (11R)-6-(2,6-Dimethylphenyl)-12-[3-(propan-2-yloxy)cyclobutyl]-11-{[1-(trifluoromethyl)cyclopropyl]methyl}-9-oxa-2λ⁶-thia-3,5,12,19-tetraazatricyclo[12.3.1.14,8]nonadeca-1(18),4(19),5,7,14,16-hexaene-2,2,13-trione

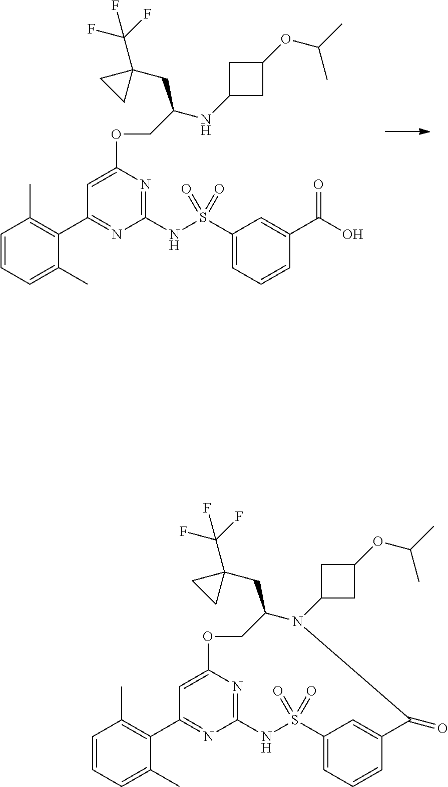 [SMILES: Cc1cccc(C)c1-c1cc(OC[C@@H](CC2(C(F)(F)F)CC2)NC2CC(OC(C)C)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC(OC(C)C)C1)[C@H](CC1(C(F)(F)F)CC1)CO2]

3-[[4-(2,6-dimethylphenyl)-6-[(2R)-2-[(3-isopropoxycyclobutyl)amino]-3-[1-(trifluoromethyl)cyclopropyl]propoxy]pyrimidin-2-yl]sulfamoyl]benzoic acid (hydrochloride salt) (62.8 mg, 0.08805 mmol), HATU (79 mg, 0.2078 mmol), and triethylamine (74 μL, 0.5309 mmol) were dissolved in DMF (2 mL) and stirred at room temperature for 16 h. The reaction mixture was filtered and purified by LC/MS utilizing a gradient of 1-99% acetonitrile in 5 mM aqueous HCl to yield (11R)-6-(2,6-dimethylphenyl)-12-[3-(propan-2-yloxy)cyclobutyl]-11-{[1-(trifluoromethyl)cyclopropyl]methyl}-9-oxa-2λ⁶-thia-3,5,12,19-tetraazatricyclo[12.3.1.14,8]nonadeca-1(18),4(19),5,7,14,16-hexaene-2,2,13-trione (24.2 mg, 18%) ESI-MS m/z calc. 658.24365, found 659.2 (M+1)⁺; Retention time: 2.79 minutes. LCMS LC method I. Compound was a mixture of cis/trans isomers

### Step 3: (11R)-6-(2,6-Dimethylphenyl)-12-(3-isopropoxycyclobutyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one, diastereomer 1 (Compound 130), and (11R)-6-(2,6-dimethylphenyl)-12-[3-(propan-2-yloxy)cyclobutyl]-11-{[1-(trifluoromethyl)cyclopropyl]methyl}-9-oxa-2λ⁶-thia-3,5,12,19-tetraazatricyclo[12.3.1.14,8]nonadeca-1(18),4(19),5,7,14,16-hexaene-2,2,13-trione, diastereomer 2 (Compound 131)

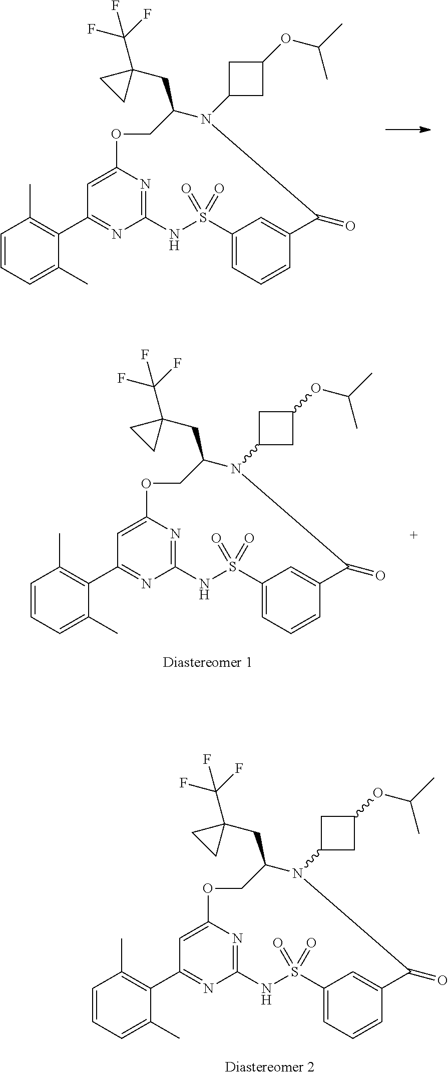 [SMILES: Cc1cccc(C)c1-c1cc2nc(n1)NS(=O)(=O)c1cccc(c1)C(=O)N(C1CC(OC(C)C)C1)[C@H](CC1(C(F)(F)F)CC1)CO2 ; Cc1cccc(C)c1-c1cc2nc(n1)NS(=O)(=O)c1cccc(c1)C(=O)N(C1CC(OC(C)C)C1)[C@H](CC1(C(F)(F)F)CC1)CO2 ; Cc1cccc(C)c1-c1cc2nc(n1)NS(=O)(=O)c1cccc(c1)C(=O)N(C1CC(OC(C)C)C1)[C@H](CC1(C(F)(F)F)CC1)CO2]

(11R)-6-(2,6-dimethylphenyl)-12-(3-isopropoxycyclobutyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (22 mg, 0.03340 mmol) was subjected to SFC separation using a ChiralPak IC (250×10 mm, 5 m) column at 35° C., a mobile phase comprised of 48% MeOH (no modifier) and 52% CO₂, with a flow of 70 mL/min, at a concentration of 22 mg/mL in MeOH (no modifier), and with an injection volume of 70 μL, at a pressure of 144 bar and utilizing a 210 nm wavelength. Two isomers were isolated: Diastereomer 1, peak 1, (11R)-6-(2,6-dimethylphenyl)-12-(3-isopropoxycyclobutyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (9.8 mg, 45%) ESI-MS m/z calc. 658.24365, found 659.3 (M+1)⁺; Retention time: 1.93 minutes (LC method A); and diastereomer 2, peak 2, (11R)-6-(2,6-dimethylphenyl)-12-[3-(propan-2-yloxy)cyclobutyl]-11-{[1-(trifluoromethyl)cyclopropyl]methyl}-9-oxa-2λ⁶-thia-3,5,12,19-tetraazatricyclo[12.3.1.14,8]nonadeca-1(18),4(19),5,7,14,16-hexaene-2,2,13-trione (3.2 mg, 15%) ESI-MS m/z calc. 658.24365, found 659.2 (M+1)⁺; Retention time: 2.01 minutes (LC method A).

### Example 106: Preparation of Compound 132

### Step 1: 3-[[4-[(2R)-2-[(2-tert-Butoxycarbonyl-2-azaspiro[3.3]heptan-6-yl)amino]-3-[1-(trifluoromethyl)cyclopropyl]propoxy]-6-(2,6-dimethylphenyl)pyrimidin-2-yl]sulfamoyl]benzoic acid

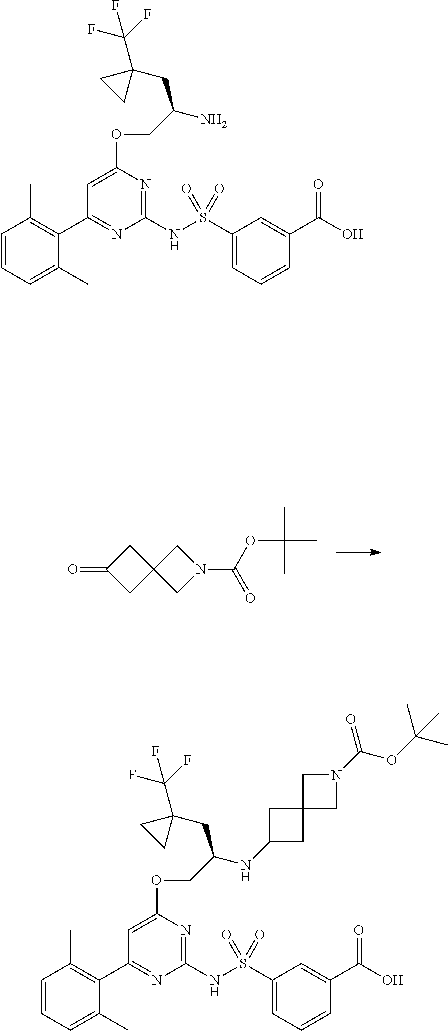 [SMILES: Cc1cccc(C)c1-c1cc(OC[C@H](N)CC2(C(F)(F)F)CC2)nc(NS(=O)(=O)c2cccc(C(=O)O)c2)n1 ; CC(C)(C)OC(=O)N1CC2(CC(=O)C2)C1 ; Cc1cccc(C)c1-c1cc(OC[C@@H](CC2(C(F)(F)F)CC2)NC2CC3(C2)CN(C(=O)OC(C)(C)C)C3)nc(NS(=O)(=O)c2cccc(C(=O)O)c2)n1]

3-[[4-[(2R)-2-amino-3-[1-(trifluoromethyl)cyclopropyl]propoxy]-6-(2,6-dimethylphenyl)pyrimidin-2-yl]sulfamoyl]benzoic acid (hydrochloride salt) (2.639 g, 4.259 mmol), tert-butyl 6-oxo-2-azaspiro[3.3]heptane-2-carboxylate (1.08 g, 5.112 mmol), and sodium triacetoxyborohydride (2.73 g, 12.88 mmol) were combined in DCM (8 mL) and stirred for 4 h. More sodium triacetoxyborohydride (2.4 g, 11.32 mmol) was added in two portions over 3 h. The reaction mixture was stirred an additional 1 h, then quenched with methanol (20 mL). The reaction mixture was partitioned between ethyl acetate and 1M HCl solution. The organics were washed twice more with 1M HCl, then brine. The organics were dried over sodium sulfate and evaporated to yield 3-[[4-[(2R)-2-[(2-tert-butoxycarbonyl-2-azaspiro[3.3]heptan-6-yl)amino]-3-[1-(trifluoromethyl)cyclopropyl]propoxy]-6-(2,6-dimethylphenyl)pyrimidin-2-yl]sulfamoyl]benzoic acid (hydrochloride salt) (3.40 g, 80%) ESI-MS m/z calc. 759.2914, found 760.8 (M+1)⁺; Retention time: 0.56 minutes. LC method D.

### Step 2: tert-Butyl 6-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]-2-azaspiro [3.3]heptane-2-carboxylate

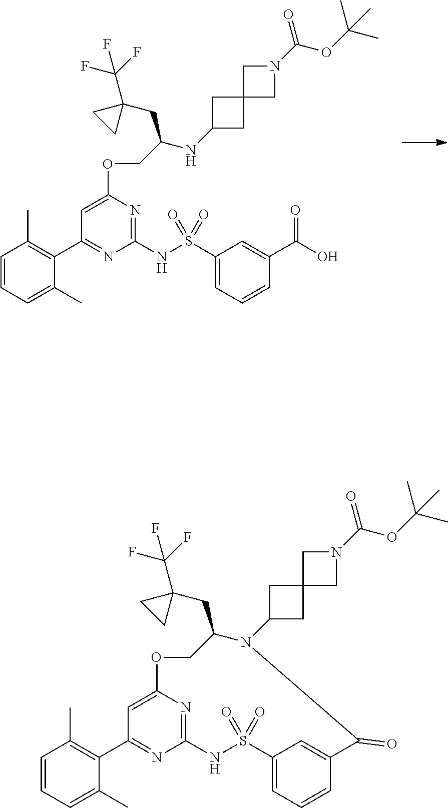 [SMILES: Cc1cccc(C)c1-c1cc(OC[C@@H](CC2(C(F)(F)F)CC2)NC2CC3(C2)CN(C(=O)OC(C)(C)C)C3)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(C1)CN(C(=O)OC(C)(C)C)C3)[C@H](CC1(C(F)(F)F)CC1)CO2]

3-[[4-[(2R)-2-[(2-tert-butoxycarbonyl-2-azaspiro[3.3]heptan-6-yl)amino]-3-[1-(trifluoromethyl)cyclopropyl]propoxy]-6-(2,6-dimethylphenyl)pyrimidin-2-yl]sulfamoyl]benzoic acid (hydrochloride salt) (1.047 g, 1.052 mmol) was dissolved in DMF (10 mL) and added dropwise to a solution of HATU (576 mg, 1.515 mmol) and triethylamine (750 μL, 5.381 mmol) in DMF (20 mL). The reaction mixture was stirred at room temperature for 16 h. There was still some starting acid so more HATU (250 mg, 0.6575 mmol) was added and the reaction was stirred an additional 6 h. The reaction was evaporated, and the resulting oil was partitioned between ethyl acetate and a 1M HCl solution. The organics were washed with 1 M HCl, brine, dried over sodium sulfate and evaporated. The crude material was purified by silica gel chromatography eluting with 10-100% ethyl acetate in hexanes to yield tert-butyl 6-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]-2-azaspiro[3.3]heptane-2-carboxylate (385 mg, 49%) ESI-MS m/z calc. 741.28076, found 742.7 (M+1)⁺; Retention time: 0.78 minutes, LC method A.

### Step 3: (11R)-6-(2,6-Dimethylphenyl)-12-(2-isopropyl-2-azaspiro[3.3]heptan-6-yl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (Compound 132)

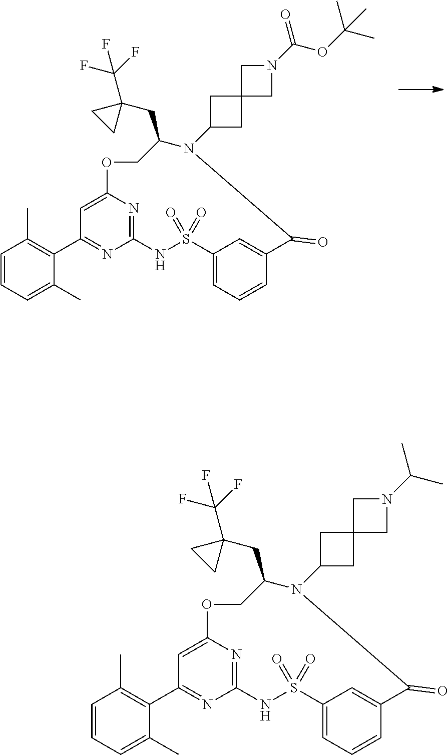 [SMILES: Cc1cccc(C)c1-c1cc2nc(n1)NS(=O)(=O)c1cccc(c1)C(=O)N(C1CC3(C1)CN(C(=O)OC(C)(C)C)C3)[C@H](CC1(C(F)(F)F)CC1)CO2 ; Cc1cccc(C)c1-c1cc2nc(n1)NS(=O)(=O)c1cccc(c1)C(=O)N(C1CC3(C1)CN(C(C)C)C3)[C@H](CC1(C(F)(F)F)CC1)CO2]

tert-butyl 6-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]-2-azaspiro[3.3]heptane-2-carboxylate (104.1 mg, 0.1403 mmol) was dissolved in DCM (1 mL) with TFA (60 μL, 0.7788 mmol) and the reaction was stirred for 1 h. The reaction was evaporated, and the resulting solid was dissolved in DCM (0.3 mL) with acetone (75 μL, 1.021 mmol) and sodium triacetoxyborohydride (154 mg, 0.7266 mmol). The reaction was stirred an additional 2 h. The mixture was partitioned between ethyl acetate and a 1M HCl solution. The organics were separated, washed with brine, dried over sodium sulfate and evaporated. The crude material was purified by LC/MS utilizing a gradient of 1-99% acetonitrile in 5 mM aqueous HCl to yield (11R)-6-(2,6-dimethylphenyl)-12-(2-isopropyl-2-azaspiro[3.3]heptan-6-yl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (hydrochloride salt) (7.5 mg, 7%) ESI-MS m/z calc. 683.2753, found 684.7 (M+1)⁺; Retention time: 1.25 minutes, LC method A.

### Example 107: Preparation of Compound 133

### Step 1: (11R)-12-(2-Azaspiro[3.3]heptan-6-yl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one

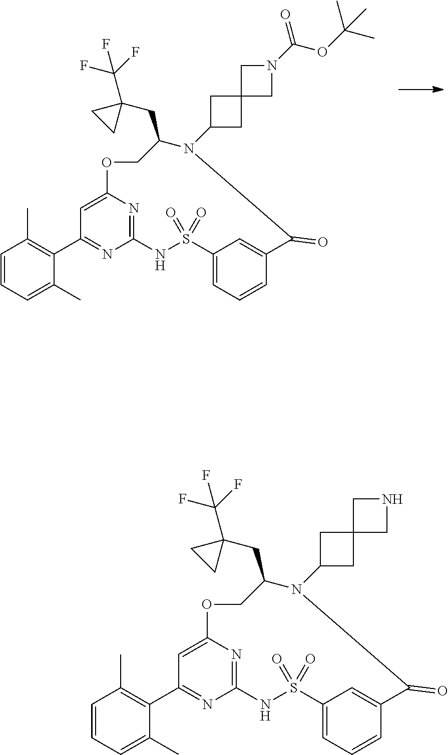 [SMILES: Cc1cccc(C)c1-c1cc2nc(n1)NS(=O)(=O)c1cccc(c1)C(=O)N(C1CC3(C1)CN(C(=O)OC(C)(C)C)C3)[C@H](CC1(C(F)(F)F)CC1)CO2 ; Cc1cccc(C)c1-c1cc2nc(n1)NS(=O)(=O)c1cccc(c1)C(=O)N(C1CC3(CNC3)C1)[C@H](CC1(C(F)(F)F)CC1)CO2]

tert-butyl 6-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]-2-azaspiro[3.3]heptane-2-carboxylate (192 mg, 0.2588 mmol) was dissolved in DCM (1.8 mL) with TFA (240 μL, 3.115 mmol) and stirred at room temperature for 6 h. The reaction was evaporated to dryness to give (11R)-12-(2-azaspiro[3.3]heptan-6-yl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (trifluoroacetate salt) (195 mg, 86%) ESI-MS m/z calc. 641.22833, found 642.7 (M+1)⁺; Retention time: 0.5 minutes, LC method D.

### Step 2: Propan-2-yl 6-[(11R)-6-(2,6-dimethylphenyl)-2,2,13-trioxo-11-{[1-(trifluoromethyl)cyclopropyl]methyl}-9-oxa-2λ⁶-thia-3,5,12,19-tetraazatricyclo[12.3.1.14,8]nonadeca-1(17),4,6,8(19),14(18),15-hexaen-12-yl]-2-azaspiro[3.3]heptane-2-carboxylate (Compound 133)

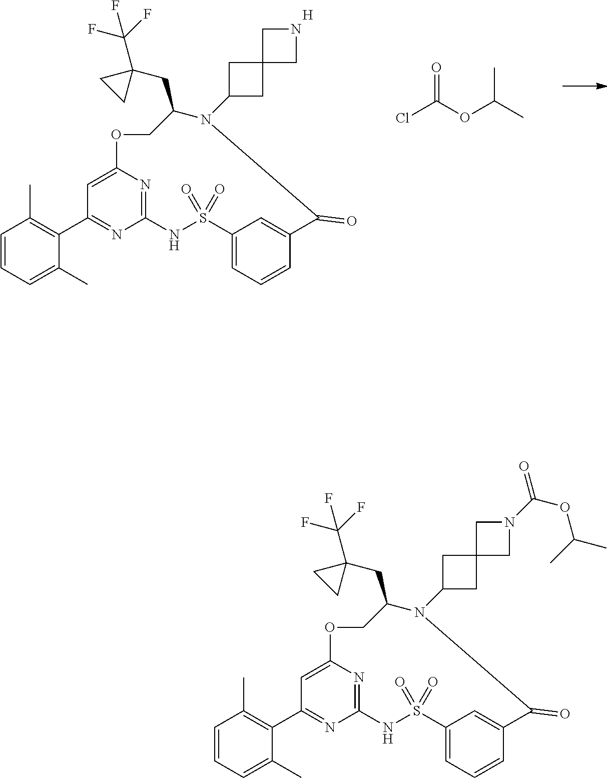

(11R)-12-(2-Azaspiro[3.3]heptan-6-yl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (trifluoroacetate salt) (40 mg, 0.05293 mmol) was dissolved in DCM (1 mL) and isopropyl chloroformate (approximately 79.40 μL of 2 M, 0.1588 mmol) and DIEA (approximately 34.20 mg, 46.09 μL, 0.2646 mmol) were added and the reaction was stirred for 10 min. The reaction was evaporated and the crude material was purified by LC/MS utilizing a gradient of 1-99% acetonitrile in 5 mM aqueous HCl to yield propan-2-yl 6-[(11R)-6-(2,6-dimethylphenyl)-2,2,13-trioxo-11-{[1-(trifluoromethyl)cyclopropyl]methyl}-9-oxa-2λ⁶-thia-3,5,12,19-tetraazatricyclo[12.3.1.14,8]nonadeca-1(17),4,6,8(19),14(18),15-hexaen-12-yl]-2-azaspiro[3.3]heptane-2-carboxylate (20.1 mg, 52%). ESI-MS m/z calc. 727.26514, found 728.8 (M+1)⁺; Retention time: 1.87 minutes; LC method A. ¹H NMR (400 MHz, DMSO-d₆) δ 13.04 (s, 1H), 8.35 (s, 1H), 7.88 (s, 1H), 7.65 (s, 2H), 7.30-7.19 (m, 1H), 7.12 (s, 2H), 6.38 (s, 1H), 5.04 (dd, J=10.8, 4.4 Hz, 1H), 4.72 (p, J=6.3 Hz, 1H), 4.29 (s, 1H), 4.09-3.99 (m, 1H), 3.95 (s, 2H), 3.90 (s, 2H), 3.83 (t, J=8.4 Hz, 1H), 3.11 (t, J=9.8 Hz, 1H), 3.03 (dd, J=11.1, 8.7 Hz, 1H), 2.47-2.36 (m, 2H), 2.22-1.79 (m, 7H), 1.49 (dd, J=16.7, 9.3 Hz, 1H), 1.16 (d, J=6.3 Hz, 6H), 0.87-0.70 (m, 2H), 0.69-0.59 (m, 1H), 0.58-0.45 (m, 1H).

### Example 108: Preparation of Compound 134 and Compound 135

### Step 1: Methyl 2-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]spiro[3.3]heptane-6-carboxylate

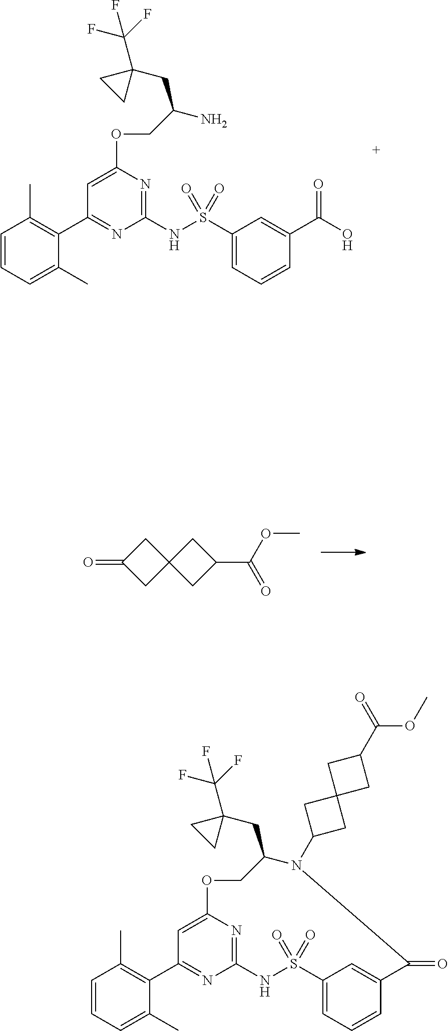 [SMILES: Cc1cccc(C)c1-c1cc(OC[C@H](N)CC2(C(F)(F)F)CC2)nc(NS(=O)(=O)c2cccc(C(=O)O)c2)n1 ; COC(=O)C1CC2(CC(=O)C2)C1 ; COC(=O)C1CC2(C1)CC(N1C(=O)c3cccc(c3)S(=O)(=O)Nc3nc(cc(-c4c(C)cccc4C)n3)OC[C@H]1CC1(C(F)(F)F)CC1)C2]

3-[[4-[(2R)-2-Amino-3-[1-(trifluoromethyl)cyclopropyl]propoxy]-6-(2,6-dimethylphenyl)pyrimidin-2-yl]sulfamoyl]benzoic acid (hydrochloride salt) (113.3 mg, 0.1885 mmol), methyl 2-oxospiro[3.3]heptane-6-carboxylate (52 mg, 0.3092 mmol), and sodium triacetoxyborohydride (124 mg, 0.5851 mmol) were combined in DCM (300 μL) and stirred at room temperature for 1 h. The reaction mixture was partitioned between ethyl acetate and a 1M HCl solution. The organics were separated, washed with brine, dried over sodium sulfate and evaporated. The crude material was dissolved in DMF (2 mL) with HATU (97.8 mg, 0.2572 mmol) and triethylamine (105 μL, 0.7533 mmol) and the reaction was stirred for 16 h at room temperature. The reaction was filtered and purified by LC/MS utilizing a gradient of 1-99% acetonitrile in 5 mM aqueous HCl to yield methyl 2-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]spiro[3.3]heptane-6-carboxylate (42.4 mg, 32%) ESI-MS m/z calc. 698.2386, found 699.19 (M+1)⁺; Retention time: 0.74 minutes, LC method D.

### Step 2: (11R)-6-(2,6-Dimethylphenyl)-12-[6-(hydroxymethyl)spiro[3.3]heptan-2-yl]-11-{[1-(trifluoromethyl)cyclopropyl]methyl}-9-oxa-2λ⁶-thia-3,5,12,19-tetraazatricyclo[12.3.1.14,8]nonadeca-1(17),4(19),5,7,14(18),15-hexaene-2,2,13-trione, diastereomer 1 (Compound 134), and (11R)-6-(2,6-dimethylphenyl)-12-[6-(hydroxymethyl)spiro[3.3]heptan-2-yl]-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-13-one, diastereomer 2 (Compound 135)

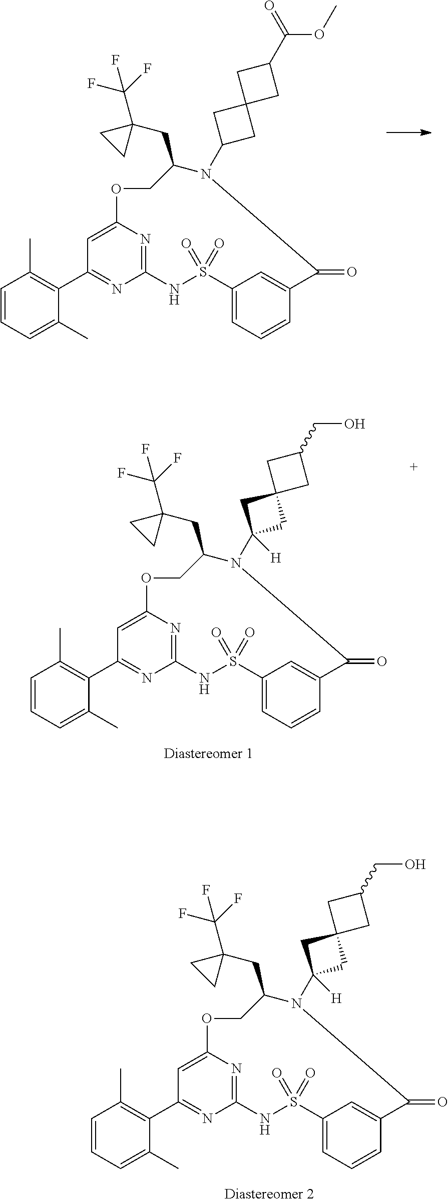

Methyl 2-[(11R)-6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]spiro[3.3]heptane-6-carboxylate (42.4 mg, 0.06068 mmol) was dissolved in lithium borohydride in THE (1 mL of 2 M, 2.000 mmol) and stirred at room temperature for 1 h. The reaction was quenched with methanol, then partitioned between ethyl acetate and a 1M HCl solution. The organics were separated, washed with brine, dried over sodium sulfate and evaporated. The crude material was purified by LC/MS utilizing a gradient of 1-99% acetonitrile in 5 mM aqueous HCl. 34 mg of product was subjected to SFC separation using a ChiralPak IC (250×10 mm, 5 m) column at 35° C., a mobile phase comprised of 36% MeOH (no modifier) and 64% CO₂, with a flow of 70 mL/min, at a concentration of 24 mg/mL in MeOH (no modifier), and with an injection volume of 70 μL, at a pressure of 165 bar and utilizing a 210 nm wavelength. Two isomers were isolated: diastereomer 1, peak 1, (11R)-6-(2,6-dimethylphenyl)-12-[6-(hydroxymethyl)spiro[3.3]heptan-2-yl]-11-{[1-(trifluoromethyl)cyclopropyl]methyl}-9-oxa-2λ⁶-thia-3,5,12,19-tetraazatricyclo[12.3.1.14,8]nonadeca-1(17),4(19),5,7,14(18),15-hexaene-2,2,13-trione (10.9 mg, 27%) ESI-MS m/z calc. 670.24365, found 671.4 (M+1)⁺; Retention time: 1.67 minutes (LC method A); and diastereomer 2, peak 2, (11R)-6-(2,6-dimethylphenyl)-12-[6-(hydroxymethyl)spiro[3.3]heptan-2-yl]-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4,6,8(19),14,16-hexaen-13-one (10.7 mg, 26%) ESI-MS m/z calc. 670.24365, found 671.4 (M+1)⁺; Retention time: 1.68 minutes (LC method A).

### Example 109: Preparation of Compound 136 and Compound 137

### Step 1: 3-[[4-[2-[(7-tert-Butoxycarbonyl-7-azaspiro[3.5]nonan-2-yl)amino]-3-[1-(trifluoromethyl)cyclopropyl]propoxy]-6-(2,6-dimethylphenyl)pyrimidin-2-yl]sulfamoyl]benzoic acid

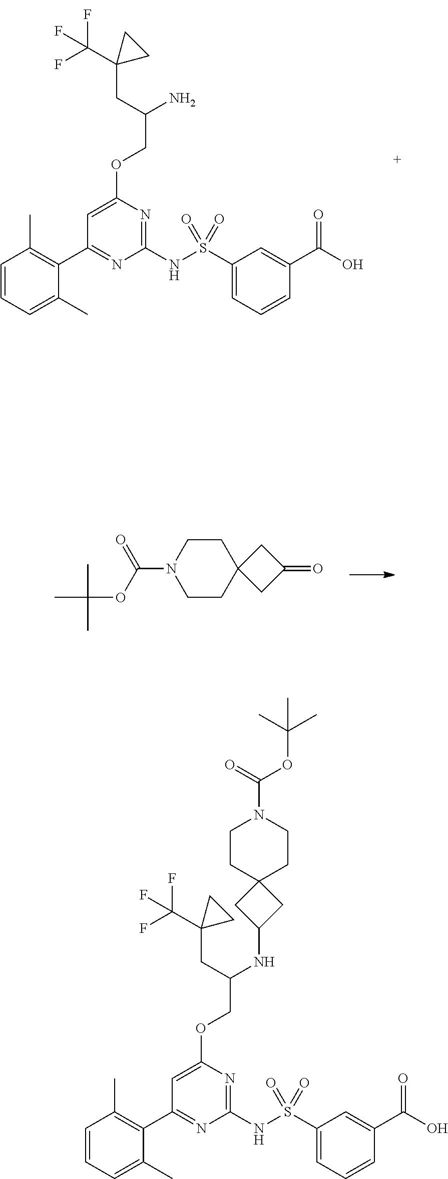

To a solution of 3-[[4-[2-amino-3-[1-(trifluoromethyl)cyclopropyl]propoxy]-6-(2,6-dimethylphenyl)pyrimidin-2-yl]sulfamoyl]benzoic acid (hydrochloride salt) (200 mg, 0.2662 mmol) in DCM (1.6 mL) was added tert-butyl 2-oxo-7-azaspiro[3.5]nonane-7-carboxylate (127.4 mg, 0.5324 mmol) followed by sodium triacetoxyborohydride (112.8 mg, 0.5322 mmol) and the mixture was stirred for 3 h 30 min then sodium triacetoxyborohydride (169.3 mg, 0.7988 mmol) was added and the resulting mixture was stirred 80 min then sodium triacetoxyborohydride (112.8 mg, 0.5322 mmol) was added. After 20 minutes, added 10 drops of saturated aqueous ammonium chloride and 1 mL of MeOH to quench. Removed volatiles by rotary evaporation. The residue was then filtered and purified using a reverse phase HPLC-MS method using a Luna C₁₈ (2) column (75×30 mm, 5 m particle size) sold by Phenomenex (pn: 00C-4252-U0-AX), and a dual gradient run from 1-99% mobile phase B over 15.0 minutes (mobile phase A=water (5 mM HCl), mobile phase B=acetonitrile, flow rate=50 mL/min, injection volume=950 μL and column temperature=25° C. Fractions containing product were concentrated by rotary evaporation to remove acetonitrile leaving a foaming aqueous solution to which was added saturated aqueous ammonium chloride and then was extracted with EtOAc. The organic phase was dried (magnesium sulfate), filtered and concentrated to give 3-[[4-[2-[(7-tert-butoxycarbonyl-7-azaspiro[3.5]nonan-2-yl)amino]-3-[1-(trifluoromethyl)cyclopropyl]propoxy]-6-(2,6-dimethylphenyl)pyrimidin-2-yl]sulfamoyl]benzoic acid (hydrochloride salt) as a white solid (133.1 mg, 61%). ESI-MS m z calc. 787.32263, found 788.2 (M+1)⁺; Retention time: 0.57 minutes, LC method D.

### Step 2: tert-Butyl 2-[6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate

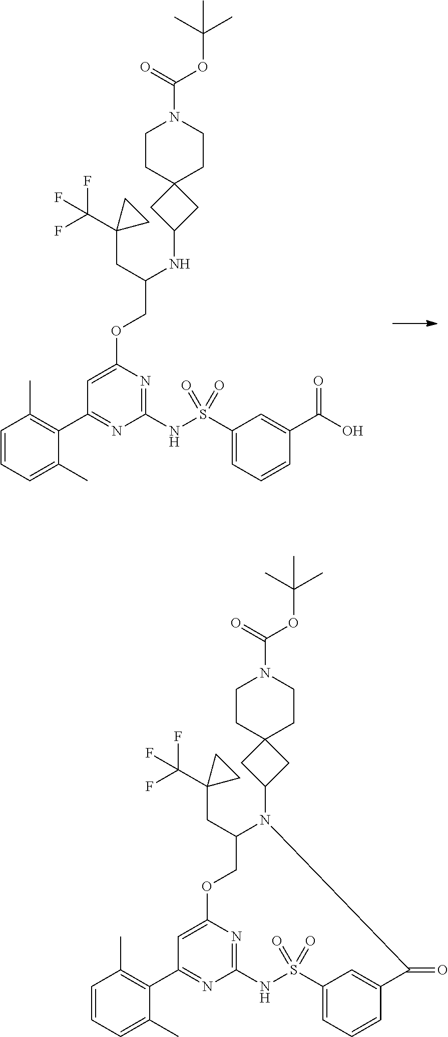 [SMILES: Cc1cccc(C)c1-c1cc(OCC(CC2(C(F)(F)F)CC2)NC2CC3(CCN(C(=O)OC(C)(C)C)CC3)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CCN(C(=O)OC(C)(C)C)CC3)C1)C(CC1(C(F)(F)F)CC1)CO2]

A solution of 3-[[4-[2-[(7-tert-butoxycarbonyl-7-azaspiro[3.5]nonan-2-yl)amino]-3-[1-(trifluoromethyl)cyclopropyl]propoxy]-6-(2,6-dimethylphenyl)pyrimidin-2-yl]sulfamoyl]benzoic acid (hydrochloride salt) (132 mg, 0.1601 mmol), [[(E)-(1-cyano-2-ethoxy-2-oxo-ethylidene)amino]oxy-tetrahydropyran-4-yl-methylene]-dimethyl-ammonium (Phosphorus Hexafluoride Ion) (109.5 mg, 0.2563 mmol), and triethylamine (91.75 μL, 0.6583 mmol) in DMF (11 mL) was stirred 2 h. The reaction was concentrated by rotary evaporation then diluted in EtOAc and washed with a 1:1 mixture of saturated aqueous ammonium chloride/brine (2×), dried (magnesium sulfate), filtered and concentrated to a light brown oil, tert-butyl 2-[6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (212.9 mg, 100%) ESI-MS m/z calc. 769.3121, found 770.1 (M+1)⁺; Retention time: 0.79 minutes, LC method D.

### Step 3: 12-(7-Azaspiro[3.5]nonan-2-yl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one

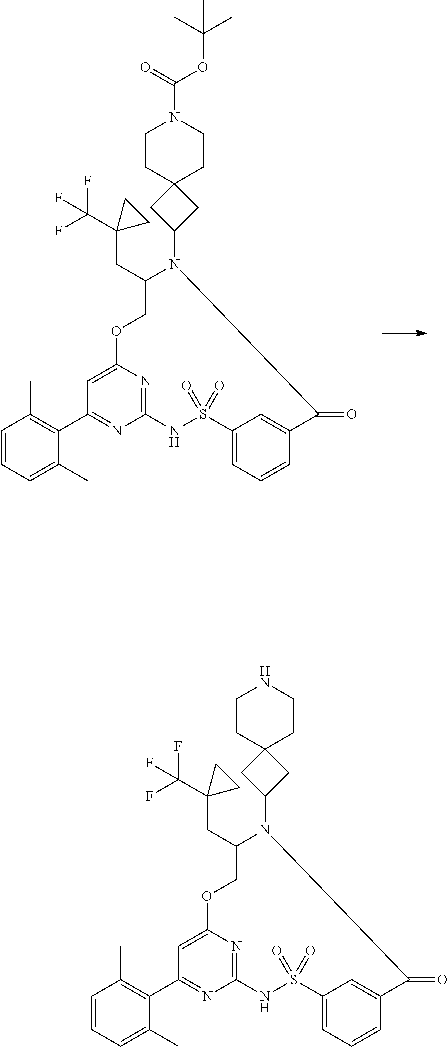 [SMILES: Cc1cccc(C)c1-c1cc2nc(n1)NS(=O)(=O)c1cccc(c1)C(=O)N(C1CC3(CCN(C(=O)OC(C)(C)C)CC3)C1)C(CC1(C(F)(F)F)CC1)CO2 ; Cc1cccc(C)c1-c1cc2nc(n1)NS(=O)(=O)c1cccc(c1)C(=O)N(C1CC3(CCNCC3)C1)C(CC1(C(F)(F)F)CC1)CO2]

Crude tert-butyl 2-[6-(2,6-dimethylphenyl)-2,2,13-trioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (212.9 mg, 0.1604 mmol) in dichloromethane (2.47 mL) was combined with HCl (1.129 mL of 4 M, 4.516 mmol) and stirred at room temperature for 30 minutes. The reaction mixture was then concentrated under a stream of nitrogen, dissolved in 1:1 DMSO/methanol, filtered and purified using a reverse phase HPLC-MS method using a Luna C₁₈ (2) column (75×30 mm, 5 m particle size) sold by Phenomenex (pn: 00C-4252-U0-AX), and a dual gradient run from 1-99% mobile phase B over 15.0 minutes (mobile phase A=water (5 mM HCl), mobile phase B=acetonitrile, flow rate=50 mL/min, injection volume=950 μL and column temperature=25° C.) giving 12-(7-azaspiro[3.5]nonan-2-yl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (hydrochloride salt) (90.3 mg, 80%) ESI-MS m/z calc. 669.25964, found 670.3 (M+1)⁺; Retention time: 0.5 minutes, LC method D.

### Step 4: 6-(2,6-Dimethylphenyl)-12-(7-methyl-7-azaspiro[3.5]nonan-2-yl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one, enantiomer 1 (Compound 136), 6-(2,6-dimethylphenyl)-12-(7-methyl-7-azaspiro[3.5]nonan-2-yl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one, enantiomer 2 (Compound 137)

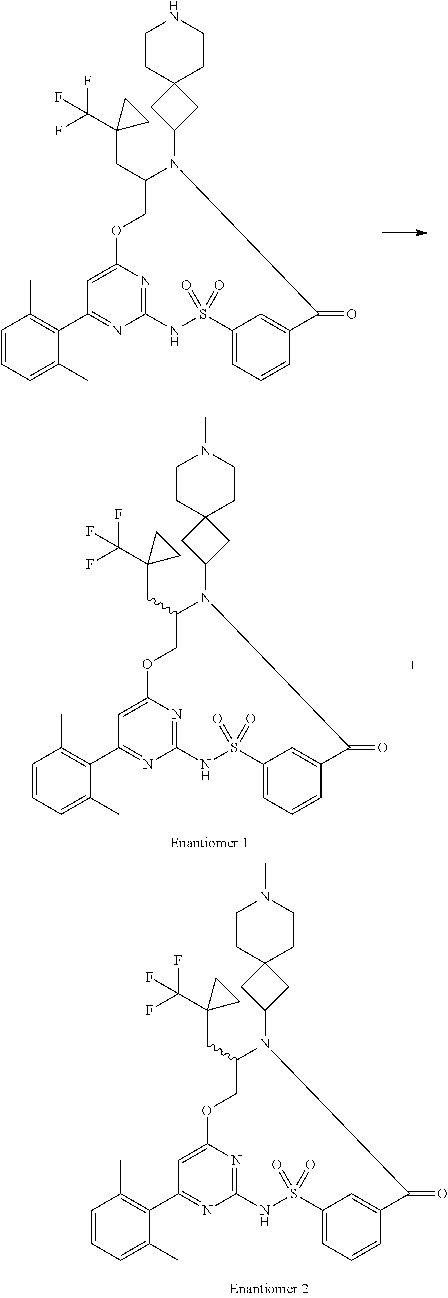 [SMILES: Cc1cccc(C)c1-c1cc2nc(n1)NS(=O)(=O)c1cccc(c1)C(=O)N(C1CC3(CCNCC3)C1)C(CC1(C(F)(F)F)CC1)CO2 ; Cc1cccc(C)c1-c1cc2nc(n1)NS(=O)(=O)c1cccc(c1)C(=O)N(C1CC3(CCN(C)CC3)C1)C(CC1(C(F)(F)F)CC1)CO2 ; Cc1cccc(C)c1-c1cc2nc(n1)NS(=O)(=O)c1cccc(c1)C(=O)N(C1CC3(CCN(C)CC3)C1)C(CC1(C(F)(F)F)CC1)CO2]

12-(7-Azaspiro[3.5]nonan-2-yl)-6-(2,6-dimethylphenyl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (hydrochloride salt) (90.3 mg, 0.1279 mmol) was dissolved in formic acid (820.9 μL) and combined with aqueous formaldehyde (2.416 mL, 87.71 mmol) and heated to 90° C. overnight in a screwcap vial. The reaction mixture was cooled to room temperature, transferred to a round bottom flask and then was then partially concentrated under reduced pressure, diluted with methanol and DMSO, added 3 drops of TEA and filtered through a plug of tissue paper in a glass pipet followed by a 0.45 μM syringe filter. The material was purified using a reverse phase HPLC-MS method using a Luna C₁₈ (2) column (75×30 mm, 5 m particle size) sold by Phenomenex (pn: 00C-4252-U0-AX), and a dual gradient run from 1-99% mobile phase B over 15.0 minutes (mobile phase A=water (5 mM HCl), mobile phase B=acetonitrile, flow rate=50 mL/min, injection volume=950 μL and column temperature=25° C.) giving 53.5 mg of racemic product as a white solid which was then subjected to chiral separation by SFC chromatography using a Chiraltek OD (250×4.6 mm column, 5 m particle size) with a gradient of 18-28% MeOH (20 mM NH₃ additive) in CO₂ mobile phase at 60 mL/min over 14.5 minutes (injection volume=100 μL of 25 mg/mL solution in DMSO) giving as the first enantiomer to elute 6-(2,6-dimethylphenyl)-12-(7-methyl-7-azaspiro[3.5]nonan-2-yl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (22.4 mg, 51%)¹H NMR (400 MHz, DMSO-d₆) δ 8.32 (s, 1H), 7.80 (t, J=4.9 Hz, 1H), 7.54 (d, J=4.8 Hz, 2H), 7.18 (t, J=7.6 Hz, 1H), 7.06 (d, J=7.6 Hz, 2H), 6.03 (s, 1H), 5.04-4.96 (m, 1H), 4.12 (t, J=15.2 Hz, 2H), 3.91 (t, J=8.8 Hz, 1H), 2.84 (s, 1H), 2.74-2.65 (m, 2H), 2.55 (s, 1H), 2.37-2.28 (m, 3H), 2.11 (d, J=16.1 Hz, 1H), 1.97 (s, 8H), 1.72 (s, 4H), 1.52 (dd, J=16.9, 8.6 Hz, 2H), 0.88-0.82 (m, 1H), 0.79 (dt, J=10.8, 5.1 Hz, 1H), 0.70 (dt, J=9.4, 4.7 Hz, 1H), 0.65 (d, J=8.8 Hz, 1H), 0.41 (s, 1H). ESI-MS m/z calc. 683.2753, found 684.2 (M+1)⁺; Retention time: 1.28 minutes (LC method A); and the second enantiomer to elute 6-(2,6-dimethylphenyl)-12-(7-methyl-7-azaspiro[3.5]nonan-2-yl)-2,2-dioxo-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (21.9 mg, 50%)¹H NMR (400 MHz, DMSO-d₆) δ 8.32 (s, 1H), 7.79 (t, J=4.7 Hz, 1H), 7.53 (d, J=4.8 Hz, 2H), 7.17 (t, J=7.5 Hz, 1H), 7.06 (d, J=7.6 Hz, 2H), 6.01 (s, 1H), 5.06-4.97 (m, 1H), 4.12 (t, J=12.3 Hz, 2H), 3.97-3.83 (m, 1H), 2.84 (s, 1H), 2.76-2.65 (m, 2H), 2.55 (s, 1H), 2.31 (d, J=9.5 Hz, 3H), 2.11 (d, J=16.2 Hz, 1H), 1.96 (dt, J=24.4, 11.0 Hz, 8H), 1.72 (s, 4H), 1.51 (t, J=8.8 Hz, 2H), 0.85 (t, J=6.6 Hz, 1H), 0.78 (q, J=5.2 Hz, 1H), 0.69 (dt, J=9.2, 4.7 Hz, 1H), 0.63 (d, J=6.0 Hz, 1H), 0.41 (s, 1H). ESI-MS m/z calc. 683.2753, found 684.2 (M+1)⁺; Retention time: 1.28 minutes (LC method A), both as a white solids.

### Example 110: Preparation of Compound 138

### Step 1: tert-Butyl 2-[[(1R)-1-(hydroxymethyl)-3,3-dimethyl-butyl]amino]-7-azaspiro[3.5]nonane-7-carboxylate

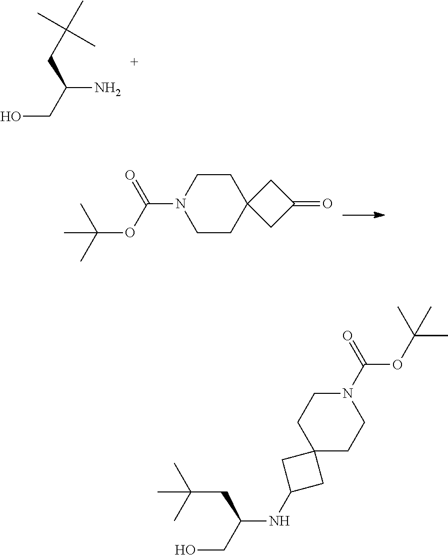

In a 25 mL flask, to a stirred milky emulsion of (2R)-2-amino-4,4-dimethyl-pentan-1-ol (hydrochloride salt) (200 mg, 1.193 mmol) in anhydrous 1,2-dichloroethane (3 mL) was added solid tert-butyl 2-oxo-7-azaspiro[3.5]nonane-7-carboxylate (370 mg, 1.546 mmol) at ambient temperature under nitrogen. The mixture was stirred for 20 min, then solid sodium triacetoxyborohydride (850 mg, 4.011 mmol) was added in 3 batches in 2 min intervals. The reaction was stirred overnight (14 h). The suspension was cooled in an ice-water bath and quenched by slow addition of aqueous hydrochloric acid (6.0 mL of 1.0 M, 6.000 mmol) to adjust pH to about 1. The emulsion was stirred for 20 min to break-up the emulsion. The resulting cooled (ice-bath) suspension was basified by slow addition of solid sodium carbonate (1.5 g, 14.15 mmol) (Caution! strong effervescence) to adjust pH to 10. The heterogeneous phases were stirred for 20 min. Then ethyl acetate (20 mL) was added and the layers were separated. The aqueous layer was extracted back with ethyl acetate (2×10 mL). The combined organics were washed with brine (10 mL), dried over sodium sulfate, filtered, and evaporated under reduced pressure to give tert-butyl 2-[[(1R)-1-(hydroxymethyl)-3,3-dimethyl-butyl]amino]-7-azaspiro[3.5]nonane-7-carboxylate (499 mg, 98%) as colorless viscous material. It was used in the subsequent reaction without further purification. ESI-MS m/z calc. 354.28824, found 355.4 (M+1)⁺; Retention time: 0.99 minutes; LC method A.

### Step 2: (11R)-12-(7-Azaspiro[3.5]nonan-2-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

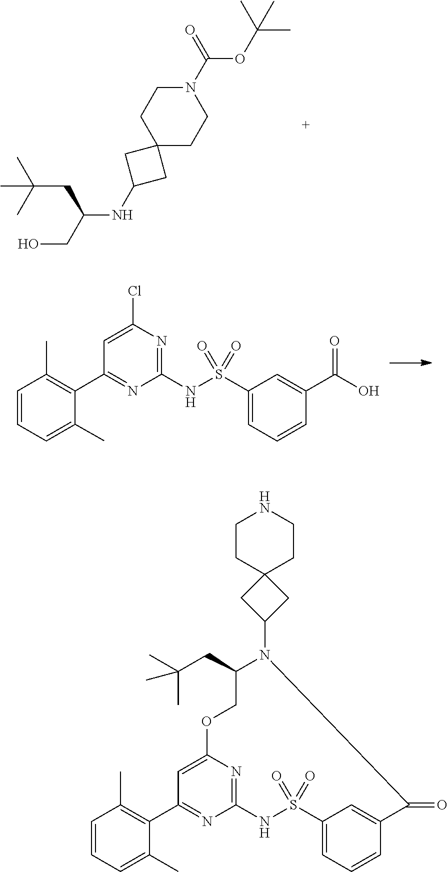 [SMILES: CC(C)(C)C[C@H](CO)NC1CC2(CCN(C(=O)OC(C)(C)C)CC2)C1 ; Cc1cccc(C)c1-c1cc(Cl)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CCNCC3)C1)[C@H](CC(C)(C)C)CO2]

A solution of tert-butyl 2-[[(1R)-1-(hydroxymethyl)-3,3-dimethyl-butyl]amino]-7-azaspiro[3.5]nonane-7-carboxylate (490 mg, 1.382 mmol) and 3-[[4-chloro-6-(2,6-dimethylphenyl)pyrimidin-2-yl]sulfamoyl]benzoic acid (547.4 mg, 1.310 mmol) in THE (5 mL) was treated with sodium tert-butoxide (631.3 mg, 6.569 mmol) at room temperature and stirred for 4 h. The mixture was then treated with 1N HCl (10 mL) and diluted with ethyl acetate (20 mL). The phases were separated and the product was extracted from the aqueous layer with ethyl acetate (15 mL×2). The combined organic layers were dried over anhydrous sodium sulfate, filtered, and concentrated in vacuo to give 3-[[4-[(2R)-2-[(7-tert-butoxycarbonyl-7-azaspiro[3.5]nonan-2-yl)amino]-4,4-dimethyl-pentoxy]-6-(2,6-dimethylphenyl)pyrimidin-2-yl]sulfamoyl]benzoic acid (1000 mg, 104%). ESI-MS m/z calc. 735.3666, found 736.7 (M+1)⁺; Retention time: 1.53 minutes; LC method A.

The product as a solution in DMF (10 mL) was added dropwise to a stirring solution of COMU (847.2 mg, 1.978 mmol) and TEA (1000 μL, 7.175 mmol) in DMF (50 mL). The reaction mixture was allowed to stir at room temperature for 4 hour, then was partitioned between 50 mL ethyl acetate and a 50 mL 1M HCl solution. The organics were separated, and the aqueous was extracted an additional 3×50 mL with ethyl acetate. The combined organics were washed with 1M HCl, then brine. The organics were dried over sodium sulfate, filtered, concentrated and evaporated in vacuo to give tert-butyl 2-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate ESI-MS m/z calc. 717.356, found 718.7 (M+1)⁺; Retention time: 2.26 (LC method A).

The product was treated with HCl (5 mL of 4 M, 20.00 mmol) (4M in dioxane) and stirred at room temperature for 2 h and then concentrated in vacuo. The resulting residue was dissolved in 1.8 mL DMSO, and purified by reverse phase HPLC using a 15 min gradient of 1-99% MeCN in water (HCl modifier) to give (11R)-12-(7-azaspiro[3.5]nonan-2-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (352 mg, 41%) ESI-MS m/z calc. 617.3036, found 618.8 (M+1)⁺; Retention time: 1.35 minutes (LC method A).

### Step 3: (11R)-6-(2,6-Dimethylphenyl)-11-(2,2-dimethylpropyl)-12-[7-(2-methoxyethyl)-7-azaspiro[3.5]nonan-2-yl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 138)

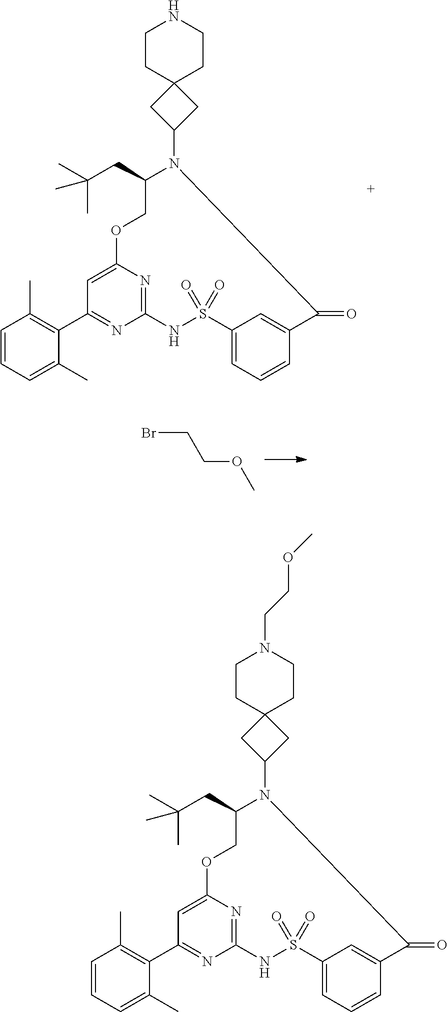

A mixture of (11R)-12-(7-azaspiro[3.5]nonan-2-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (44.4 mg, 0.06786 mmol) and TEA (70 μL, 0.5022 mmol) was heated to 55° C. and treated with 1-bromo-2-methoxy-ethane (8 μL, 0.08513 mmol). The reaction was stirred for 16 h. The solutions were filtered and the filtrate dissolved in 0.8 mL DMSO, and purified by reverse phase HPLC using a 15 min gradient of 1-99% MeCN in water (HCl modifier) to give (11R)-6-(2,6-dimethylphenyl)-11-(2,2-dimethylpropyl)-12-[7-(2-methoxyethyl)-7-azaspiro[3.5]nonan-2-yl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (19.4 mg, 38%). ESI-MS m/z calc. 675.34546, found 676.7 (M+1)⁺; Retention time: 1.4 minutes; LC method A.

### Example 111: Preparation of Compound 139

### Step 1: (2R)-2-[(3-benzyloxycyclobutyl)amino]-4,4-dimethyl-pentan-1-ol

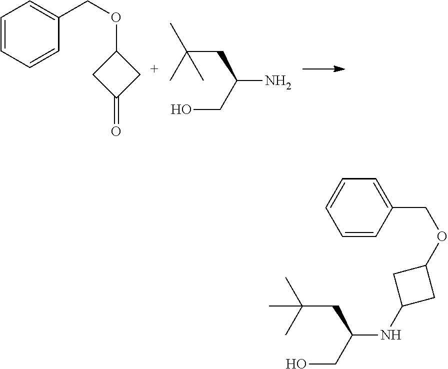

Into a solution of 3-benzyloxycyclobutanone (3.014 g, 16.591 mmol) in anhydrous DCE (30 mL) was added (2R)-2-amino-4,4-dimethyl-pentan-1-ol (hydrochloride salt) (3.527 g, 21.035 mmol). The reaction was stirred at rt for 30 minutes, before sodium triacetoxyborohydride (6.496 g, 29.118 mmol) was added. The reaction mixture was stirred at rt for 2 hours. The reaction was quenched with 2 N sodium carbonate (aqueous) (30 mL). The aqueous layer was extracted with chloroform (3×30 mL). The combined organic layers were washed with brine (20 mL), dried over anhydrous sodium sulfate and concentrated under vacuum. The residue was purified by silica gel chromatography using 0 to 10% methanol in dichloromethane (buffered with 0.3% NH4OH) to furnish (2R)-2-[(3-benzyloxycyclobutyl)amino]-4,4-dimethyl-pentan-1-ol (4.905 g, 100%) as a clear gel. The product was a mixture of diastereomers. ESI-MS m/z calc. 291.21982, found 292.3 (M+1)⁺; Retention time: 2.29 minutes; LC method T.

### Step 2: 3-[[4-[(2R)-2-[(3-Benzyloxycyclobutyl)amino]-4,4-dimethyl-pentoxy]-6-(2,6-dimethylphenyl)pyrimidin-2-yl]sulfamoyl]benzoic acid

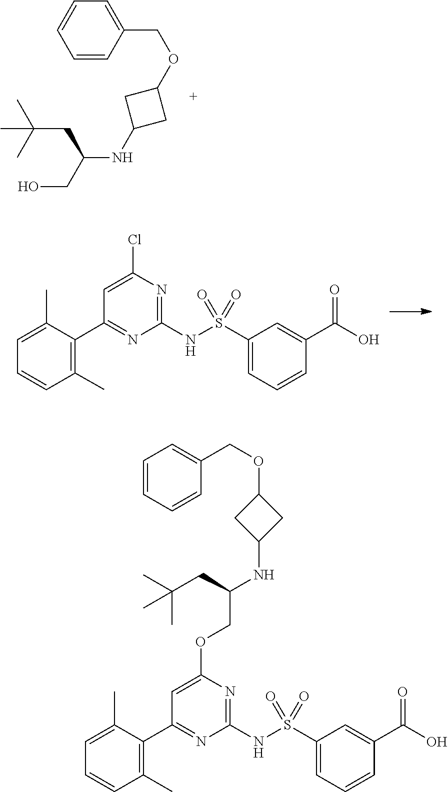 [SMILES: CC(C)(C)C[C@H](CO)NC1CC(OCc2ccccc2)C1 ; Cc1cccc(C)c1-c1cc(Cl)nc(NS(=O)(=O)c2cccc(C(=O)O)c2)n1 ; Cc1cccc(C)c1-c1cc(OC[C@@H](CC(C)(C)C)NC2CC(OCc3ccccc3)C2)nc(NS(=O)(=O)c2cccc(C(=O)O)c2)n1]

Into a solution of (2R)-2-[(3-benzyloxycyclobutyl)amino]-4,4-dimethyl-pentan-1-ol (4.905 g, 16.663 mmol) and 3-[[4-chloro-6-(2,6-dimethylphenyl)pyrimidin-2-yl]sulfamoyl]benzoic acid (6.964 g, 16.666 mmol) in anhydrous THE (50 mL) was added sodium tert-butoxide (8.17 g, 83.312 mmol) at 0° C. The reaction was stirred at rt for 3 hours. The reaction was quenched with 1 N HCl (aqueous) (50 mL) at 0° C. Two layers were separated and the aqueous layer was extracted with ethyl acetate (2×50 mL). The combined organic layers were washed with brine (50 mL), dried over anhydrous sodium sulfate, and then concentrated under vacuum to furnish 3-[[4-[(2R)-2-[(3-benzyloxycyclobutyl)amino]-4,4-dimethyl-pentoxy]-6-(2,6-dimethylphenyl)pyrimidin-2-yl]sulfamoyl]benzoic acid (hydrochloride salt) (11.758 g, 90%) as an off white solid. The crude product was used in the next step reaction without purification. ESI-MS m/z calc. 672.29816, found 673.4 (M+1)⁺; Retention time: 2.74 minutes; LC method T.

### Step 3: (11R)-12-(3-Benzyloxycyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

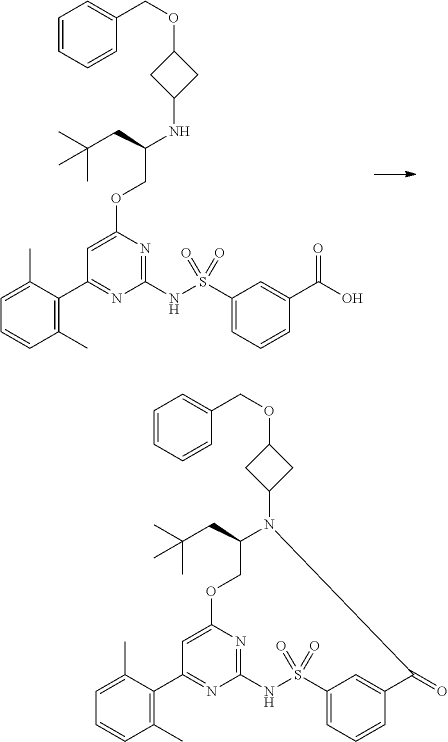 [SMILES: Cc1cccc(C)c1-c1cc(OC[C@@H](CC(C)(C)C)NC2CC(OCc3ccccc3)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC(OCc3ccccc3)C1)[C@H](CC(C)(C)C)CO2]

Into a solution of 3-[[4-[(2R)-2-[(3-benzyloxycyclobutyl)amino]-4,4-dimethyl-pentoxy]-6-(2,6-dimethylphenyl)pyrimidin-2-yl]sulfamoyl]benzoic acid (hydrochloride salt) (15.4 g, 19.541 mmol) in DMF (300 mL) was added COMU (12.55 g, 29.304 mmol) at 0° C. DIEA (10.091 g, 13.6 mL, 78.078 mmol) was added to the reaction dropwise. The reaction was slowly raised to rt and stirred overnight. The reaction was quenched with 10% citric acid aqueous solution (300 mL). The aqueous solution was extracted with ethyl acetate (3×300 mL). The combined organic layers were washed with brine (3×300 mL), dried over anhydrous sodium sulfate and concentrated under vacuum. The reside was purified by silica gel chromatography using 20 to 50% acetone in hexane (loaded with toluene, 330 g silica gel column) to furnish (11R)-12-(3-benzyloxycyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (14.24 g, 82%) as a red foamy solid. ESI-MS m/z calc. 654.2876, found 655.4 (M+1)⁺; Retention time: 3.68 minutes, LC method T.

### Step 4: (11R)-6-(2,6-Dimethylphenyl)-11-(2,2-dimethylpropyl)-12-(3-hydroxycyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one

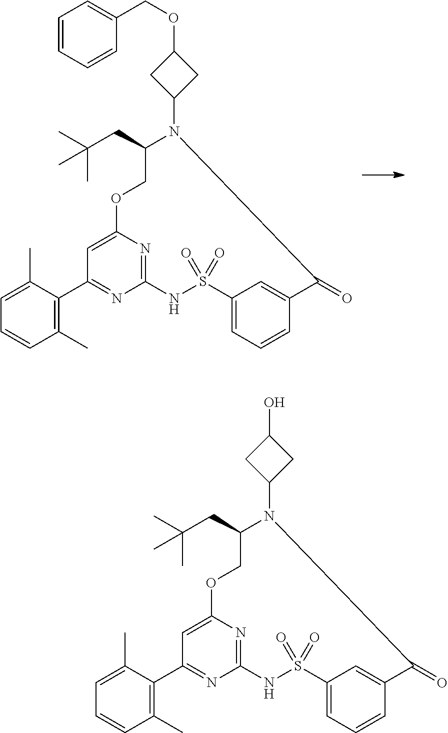 [SMILES: Cc1cccc(C)c1-c1cc2nc(n1)NS(=O)(=O)c1cccc(c1)C(=O)N(C1CC(OCc3ccccc3)C1)[C@H](CC(C)(C)C)CO2 ; Cc1cccc(C)c1-c1cc2nc(n1)NS(=O)(=O)c1cccc(c1)C(=O)N(C1CC(O)C1)[C@H](CC(C)(C)C)CO2]

Into a solution of (11R)-12-(3-benzyloxycyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (62 mg, 0.0701 mmol) in methanol (5 mL) was added 10% Palladium on carbon (10 mg, 0.0094 mmol). The reaction mixture was purged with nitrogen and then it was hydrogenated under 1 atm hydrogen atmosphere for 16 hours. Activated carbon (60 mg) was added to the reaction mixture. The solution was heated at 60° C. for 1 hour. After cooling down to rt, the solid was removed by filtration through a pad of Celite. Another batch of 10% Palladium on carbon (10 mg, 0.0094 mmol) was added. The reaction was stirred under 1 atm hydrogen atmosphere for 3 hours. 20% Pd(OH)₂ (20 mg, 0.0285 mmol) was added. The reaction was stirred under 1 atm hydrogen atmosphere for 2 days. Water (5 mL) was added. The reaction was added. The catalyst was removed by filtration through a pad of Celite, and washed with methanol (5 mL). The combined filtrate was concentrated under vacuum. To the residue was added 10% citric acid (10 mL), and extracted with ethyl acetate (3×20 mL). The combined organic layers were washed with brine (20 mL), dried over anhydrous magnesium sulfate and concentrated under vacuum to furnish (11R)-6-(2,6-dimethylphenyl)-11-(2,2-dimethylpropyl)-12-(3-hydroxycyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one (39 mg, 94%) as a clear gel (mixture of diastereomers). ESI-MS m/z calc. 564.24066, found 565.2 (M+1)⁺; Retention time: 2.88 minutes; LC method T.

### Step 5: (11R)-6-(2,6-Dimethylphenyl)-11-(2,2-dimethylpropyl)-2,2-dioxo-12-(3-oxocyclobutyl)-9-oxa-2λ⁶-thia-3,5,12,19-tetrazatricyclo[12.3.1.14,8]nonadeca-1(18),4(19),5,7,14,16-hexaen-13-one

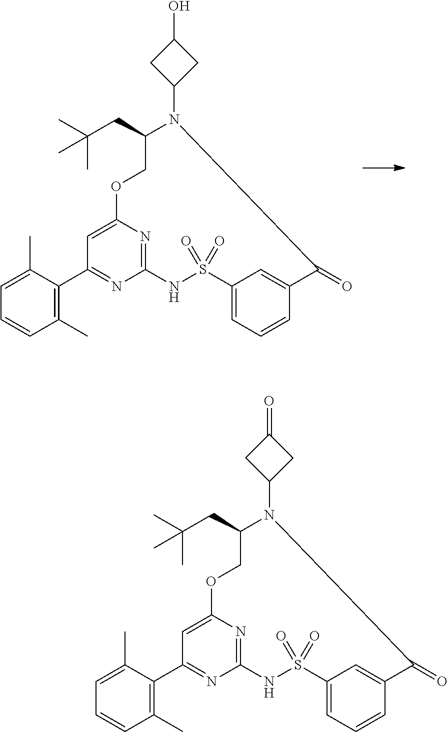 [SMILES: Cc1cccc(C)c1-c1cc2nc(n1)NS(=O)(=O)c1cccc(c1)C(=O)N(C1CC(O)C1)[C@H](CC(C)(C)C)CO2 ; Cc1cccc(C)c1-c1cc2nc(n1)NS(=O)(=O)c1cccc(c1)C(=O)N(C1CC(=O)C1)[C@H](CC(C)(C)C)CO2]

Into a solution of (11R)-6-(2,6-dimethylphenyl)-11-(2,2-dimethylpropyl)-12-(3-hydroxycyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one (4.3 g, 7.2340 mmol) in DCM (100 mL) was added DMP (3.7 g, 8.7235 mmol) at 0° C. The reaction was stirred at rt for 2 days. The reaction was diluted with ethyl acetate (100 mL) and a 1:1 mixture of saturated sodium bicarbonate and 10% sodium thiosulfate (100 mL). Two layers were separated, and the aqueous layer was extracted with ethyl acetate (2×100 mL). The combined organic layers were washed with brine (100 mL), dried over anhydrous magnesium sulfate and concentrated under vacuum. To the residue was added acetic acid (1 mL) and then concentrated again. The residue was purified by silica gel chromatography using 0 to 50% acetone in hexane followed by 0 to 10% methanol in DCM. The correct fractions were combined and then concentrated under vacuum. The residue was triturated with diethyl ether (40 mL) to furnish (11R)-6-(2,6-dimethylphenyl)-11-(2,2-dimethylpropyl)-2,2-dioxo-12-(3-oxocyclobutyl)-9-oxa-2λ⁶-thia-3,5,12,19-tetrazatricyclo[12.3.1.14,8]nonadeca-1(18),4(19),5,7,14,16-hexaen-13-one (1.8521 g, 44%) as a white solid. ESI-MS m/z calc. 562.225, found 563.5 (M+1)⁺; Retention time: 2.47 minutes; LC method W. ¹H NMR (500 MHz, DMSO-d₆) δ 13.08 (s, 1H), 8.56 (s, 1H), 7.95 (s, 1H), 7.79-7.56 (m, 2H), 7.26 (t, J=7.6, 7.6 Hz, 1H), 7.12 (s, 2H), 6.44 (s, 1H), 5.18 (dd, J=10.8, 4.6 Hz, 1H), 4.48-4.21 (m, 2H), 3.83 (ddd, J=12.5, 8.1, 4.7 Hz, 1H), 3.76-3.60 (m, 2H), 3.49-3.33 (m, 2H, overlaps with water peak in DMSO-d₆), 2.30-1.75 (m, 6H), 1.67 (dd, J=15.3, 8.2 Hz, 1H), 1.43 (d, J=15.1 Hz, 1H), 0.49 (s, 9H).

### Step 6: (11R)-6-(2,6-Dimethylphenyl)-11-(2,2-dimethylpropyl)-12-(3-hydroxy-3-methyl-cyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 139)

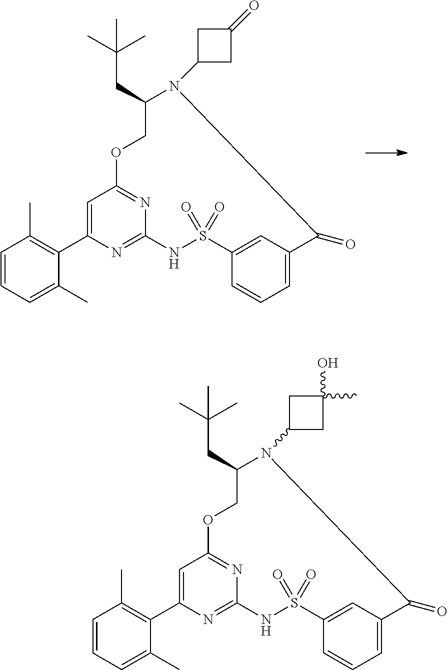

(11R)-6-(2,6-Dimethylphenyl)-11-(2,2-dimethylpropyl)-2,2-dioxo-12-(3-oxocyclobutyl)-9-oxa-2λ⁶-thia-3,5,12,19-tetrazatricyclo[12.3.1.14,8]nonadeca-1(18),4(19),5,7,14,16-hexaen-13-one (50 mg, 0.08886 mmol) was dissolved in THE (0.50 mL) and cooled to 0° C. for the addition of a solution of bromo(methyl)magnesium (50 μL of 3.0 M, 0.1500 mmol) in diethyl ether. Stirring was continued at 0° C. for 15 minutes, and the reaction mixture was allowed to stir overnight at room temperature. The reaction mixture was filtered, and the product was isolated by UV-triggered reverse phase HPLC eluting with a 10-99% acetonitrile/water gradient over 15 minutes with 0.5 mM HCl acid modifier in the aqueous phase, to give (11R)-6-(2,6-dimethylphenyl)-11-(2,2-dimethylpropyl)-12-(3-hydroxy-3-methyl-cyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one (36.9 mg, 72%) ESI-MS m/z calc. 578.2563, found 579.2 (M+1)⁺; Retention time: 1.69 minutes; LC method A. Single isomer of unknown configuration

### Example 112: Preparation of Compound 140

### Step 1: (11R)-12-(3-Benzyl-3-hydroxy-cyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 140)

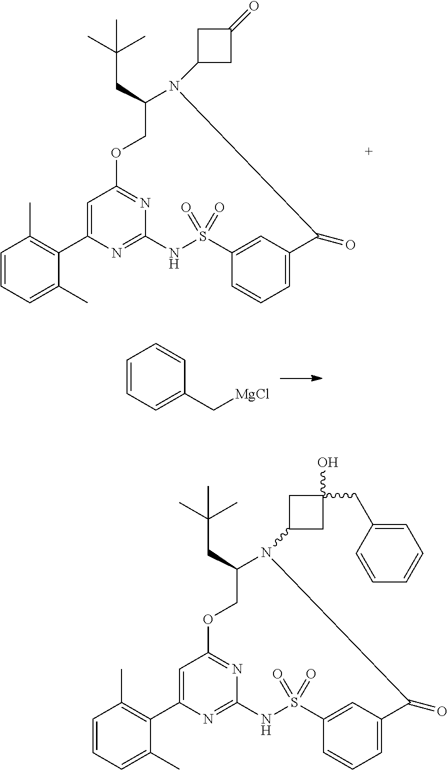

(11R)-6-(2,6-Dimethylphenyl)-11-(2,2-dimethylpropyl)-2,2-dioxo-12-(3-oxocyclobutyl)-9-oxa-2λ⁶-thia-3,5,12,19-tetrazatricyclo[12.3.1.14,8]nonadeca-1(18),4(19),5,7,14,16-hexaen-13-one (50 mg, 0.08886 mmol) was dissolved in THE (0.50 mL). The solution was cooled to 0° C. for the addition of a solution of benzyl(chloro)magnesium (125 μL of 1.0 M, 0.1250 mmol) in THF. Stirring was continued at 0° C. for 15 minutes, and the reaction mixture was allowed to stir overnight at room temperature. The reaction mixture was filtered, and the product was isolated by UV-triggered reverse phase HPLC eluting with a 10-99% acetonitrile/water gradient over 15 minutes with 0.5 mM HCl acid modifier in the aqueous phase. (11R)-12-(3-benzyl-3-hydroxy-cyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (11.3 mg, 19%) was obtained. ESI-MS m/z calc. 654.2876, found 655.2 (M+1)⁺; Retention time: 2.0 minutes; LC method A.

### Example 113: Preparation of Compound 141

### Step 1: 3-[[4-(2,6-Dimethylphenyl)-6-[(2R)-2-[(3-methoxycarbonylcyclobutyl)amino]-4,4-dimethyl-pentoxy]pyrimidin-2-yl]sulfamoyl]benzoic acid

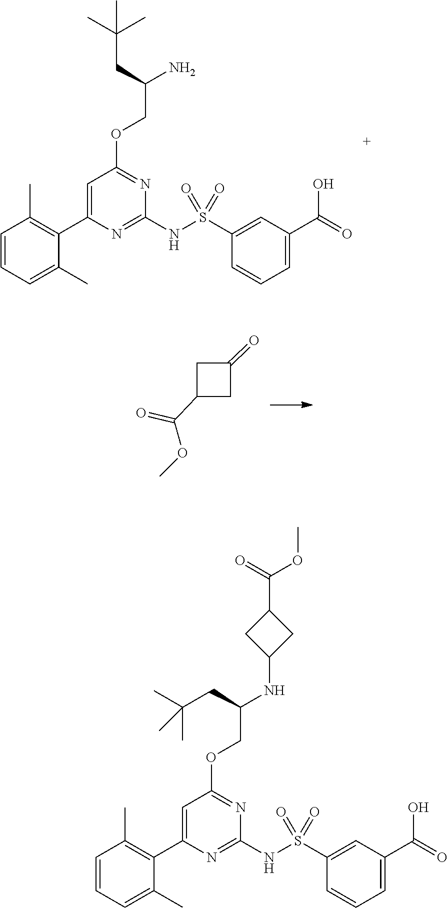

A 100 mL flask was charged under nitrogen with 3-[[4-[(2R)-2-amino-4,4-dimethyl-pentoxy]-6-(2,6-dimethylphenyl)pyrimidin-2-yl]sulfamoyl]benzoic acid (hydrochloride salt) (1.614 g, 2.939 mmol), anhydrous DCM (5 mL) and DIEA (0.56 mL, 3.215 mmol). The mixture was stirred until almost complete dissolution. acetic acid (0.19 mL, 3.341 mmol) was added followed quickly by methyl 3-oxocyclobutanecarboxylate (0.46 mL). The solution was stirred at room temperature for 20 min. sodium triacetoxyborohydride (1.31 g, 6.181 mmol) was added and the suspension was stirred at room temperature for 7 h. The mixture was stored in the freezer at −20° C. for 2 days. It was warmed up to room temperature. More methyl 3-oxocyclobutanecarboxylate (0.46 mL) and sodium triacetoxyborohydride (1.25 g, 5.898 mmol) were added and the reaction was stirred for an additional 6 hours. The reaction was cooled down in ice and quenched by slow addition of aqueous 1N HCl (50 mL). EtOAc (30 mL) and brine (15 mL) were added and the two phases were separated. The aqueous phase was further extracted with EtOAc (2×20 mL). The combined extracts were dried over sodium sulfate and the solvents evaporated. The residue was diluted in diethyl ether (100 mL) and the resulting suspension was stirred for 10 minutes. The product was filtered, washed with ether and dried to give 3-[[4-(2,6-dimethylphenyl)-6-[(2R)-2-[(3-methoxycarbonylcyclobutyl)amino]-4,4-dimethyl-pentoxy]pyrimidin-2-yl]sulfamoyl]benzoic acid (hydrochloride salt) (1.503 g, 77%) as a white solid. ESI-MS m/z calc. 624.2618, found 625.29 (M+1)⁺; Retention time: 1.19 minutes; LC method A.

### Step 2: Methyl 3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]cyclobutane-1-carboxylate, diastereomer 1, and methyl 3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]cyclobutane-1-carboxylate, diastereomer 2

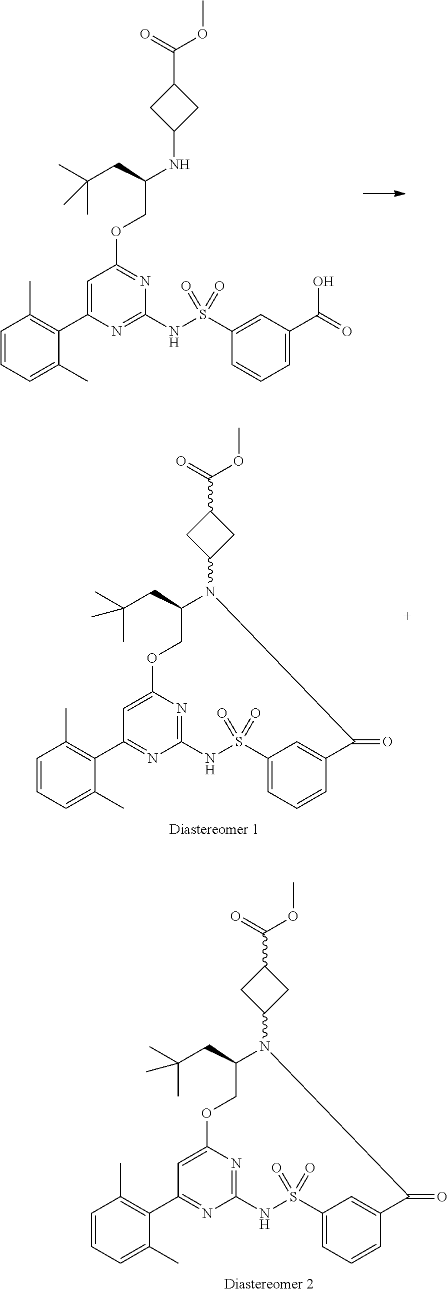 [SMILES: COC(=O)C1CC(N[C@@H](COc2cc(-c3c(C)cccc3C)nc(NS(=O)(=O)c3cccc(C(=O)O)c3)n2)CC(C)(C)C)C1 ; COC(=O)C1CC(N2C(=O)c3cccc(c3)S(=O)(=O)Nc3nc(cc(-c4c(C)cccc4C)n3)OC[C@H]2CC(C)(C)C)C1 ; COC(=O)C1CC(N2C(=O)c3cccc(c3)S(=O)(=O)Nc3nc(cc(-c4c(C)cccc4C)n3)OC[C@H]2CC(C)(C)C)C1]

A 250 mL round bottom flask was charged under nitrogen with COMU (2.25 g, 5.254 mmol), anhydrous DMF (70 mL) and DIEA (2.0 mL, 11.48 mmol). A solution of crude 3-[[4-(2,6-dimethylphenyl)-6-[(2R)-2-[(3-methoxycarbonylcyclobutyl)amino]-4,4-dimethyl-pentoxy]pyrimidin-2-yl]sulfamoyl]benzoic acid (hydrochloride salt) (1.5 g, 2.269 mmol in anhydrous DMF (20 mL) was added dropwise through syringe over a period of 5 minutes. The mixture was stirred at room temperature for 21 h (two isomers visible, ratio 70:30). The reaction was concentrated to a third and diluted with Ethyl acetate (50 mL), 1N aqueous HCl (40 mL) and brine (20 mL). The two phases were separated, and the aqueous phase was further extracted with EtOAc (2×15 mL). The combined extracts were dried over sodium sulfate and the solvents evaporated. The residue was dissolved in DCM and purified by flash chromatography on silica gel (80 g column) using a gradient of ethyl acetate (20 to 100% over 30 min) in hexanes to give diastereomer 1, less polar, minor isomer, eluted around 50-55% EtOAc, methyl 3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]cyclobutane-1-carboxylate (102 mg, 25%). ESI-MS m/z calc. 606.2512, found 607.41 (M+1)⁺; Retention time: 1.81 minutes (LC method A); and diastereomer 2, more polar, major isomer, methyl 3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]cyclobutane-1-carboxylate (331 mg, 34%). ESI-MS m/z calc. 606.2512, found 607.18 (M+1)⁺; Retention time: 1.81 minutes (LC method A).

### Step 3: (11R)-6-(2,6-Dimethylphenyl)-11-(2,2-dimethylpropyl)-12-[3-(2-hydroxypropan-2-yl)cyclobutyl]-9-oxa-2λ⁶-thia-3,5,12,19-tetraazatricyclo[12.3.1.14,8]nonadeca-1(17),4(19),5,7,14(18),15-hexaene-2,2,13-trione (Compound 141)

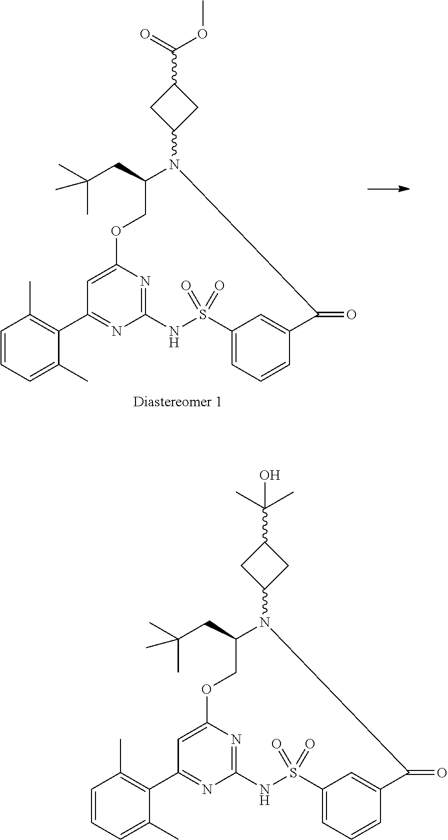 [SMILES: COC(=O)C1CC(N2C(=O)c3cccc(c3)S(=O)(=O)Nc3nc(cc(-c4c(C)cccc4C)n3)OC[C@H]2CC(C)(C)C)C1 ; Cc1cccc(C)c1-c1cc2nc(n1)NS(=O)(=O)c1cccc(c1)C(=O)N(C1CC(C(C)(C)O)C1)[C@H](CC(C)(C)C)CO2]

A 4 mL vial was charged under nitrogen with methyl 3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]cyclobutane-1-carboxylate (Diastereomer 1, 21 mg, 0.03461 mmol) and anhydrous THE (250 μL). The solution was cooled down in ice. MeMgBr (0.04 mL of 3 M, 0.1200 mmol) (3 M in diethyl ether) was added. The mixture was stirred for a few minutes in the ice bath, then the bath was removed, and the mixture was stirred at room temperature for 18 hours. The mixture was cooled down in ice and quenched by adding an aqueous saturated solution of ammonium chloride (2 mL). The product was extracted with EtOAc (3×2 mL). The combined extracts were dried over sodium sulfate and the solvents evaporated to give a crude residue that was dissolved in DMSO (1 mL). The solution was microfiltered through a syringe filter disc and purified by reverse phase preparative HPLC (C₁₈) using a gradient of acetonitrile in water (1 to 99% over 15 min) and HCl as a modifier to give (11R)-6-(2,6-dimethylphenyl)-11-(2,2-dimethylpropyl)-12-[3-(2-hydroxypropan-2-yl)cyclobutyl]-9-oxa-2λ⁶-thia-3,5,12,19-tetraazatricyclo[12.3.1.14,8]nonadeca-1(17),4(19),5,7,14(18),15-hexaene-2,2,13-trione (11 mg, 52%) as a white solid. ESI-MS m/z calc. 606.2876, found 607.68 (M+1)⁺; Retention time: 1.74 minutes (LC method A), ¹H NMR (400 MHz, DMSO-d₆) δ 13.39-11.78 (broad m, 1H), 8.42 (s, 1H), 7.92 (s, 1H), 7.68 (s, 2H), 7.33-7.19 (m, 1H), 7.12 (s, 2H), 6.40 (s, 1H), 5.07 (dd, J=11.3, 3.6 Hz, 1H), 4.25 (s, 1H), 4.13 (t, J=11.0 Hz, 1H), 3.89 (p, J=8.7 Hz, 1H), 3.72-3.61 (m, 1H), 2.93 (q, J=10.1 Hz, 1H), 2.83 (q, J=10.1 Hz, 1H), 2.39-2.28 (m, 1H), 2.28-1.68 (m, 8H), 1.51 (dd, J=15.3, 8.0 Hz, 1H), 1.36 (d, J=14.8 Hz, 1H), 1.08 (s, 6H), 0.49 (s, 9H).

### Example 114: Preparation of Compound 142

### Step 1: (11R)-6-(2,6-dimethylphenyl)-11-(2,2-dimethylpropyl)-12-[3-(2-hydroxypropan-2-yl)cyclobutyl]-9-oxa-2λ⁶-thia-3,5,12,19-tetraazatricyclo[12.3.1.14,8]nonadeca-1(17),4(19),5,7,14(18),15-hexaene-2,2,13-trione (Compound 142)

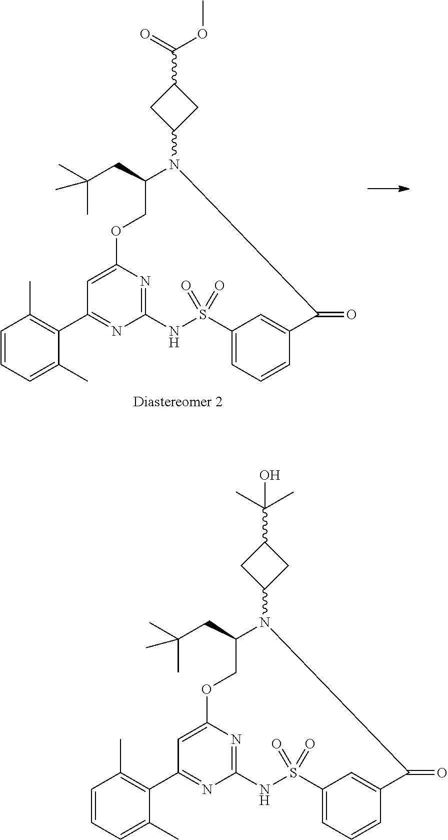 [SMILES: COC(=O)C1CC(N2C(=O)c3cccc(c3)S(=O)(=O)Nc3nc(cc(-c4c(C)cccc4C)n3)OC[C@H]2CC(C)(C)C)C1 ; Cc1cccc(C)c1-c1cc2nc(n1)NS(=O)(=O)c1cccc(c1)C(=O)N(C1CC(C(C)(C)O)C1)[C@H](CC(C)(C)C)CO2]

A 4 mL vial was charged under nitrogen with methyl 3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]cyclobutane-1-carboxylate (diastereomer 2, 28 mg, 0.04615 mmol) and anhydrous THE (0.35 mL). The solution was cooled down in ice. MeMgBr (0.05 mL of 3 M, 0.1500 mmol) (3 M in diethyl ether) was added. The mixture was stirred for a few minutes in the ice bath, then the bath was removed, and the mixture was stirred at room temperature for 4 hours. The mixture was cooled down in ice and quenched by adding an aqueous saturated solution of ammonium chloride (2 mL). The product was extracted with EtOAc (3×2 mL). The combined extracts were dried over sodium sulfate and the solvents evaporated to give a crude residue that was dissolved in DMSO (1 mL). The solution was microfiltered through a syringe filter disc and purified by reverse phase preparative HPLC (C₁₈) using a gradient of acetonitrile in water (1 to 99% over 15 min) and HCl as a modifier to give (11R)-6-(2,6-dimethylphenyl)-11-(2,2-dimethylpropyl)-12-[3-(2-hydroxypropan-2-yl)cyclobutyl]-9-oxa-2λ⁶-thia-3,5,12,19-tetraazatricyclo[12.3.1.14,8]nonadeca-1(17),4(19),5,7,14(18),15-hexaene-2,2,13-trione (11.9 mg, 42%) as a white solid. ESI-MS m/z calc. 606.2876, found 607.53 (M+1)⁺; Retention time: 1.84 minutes (LC method A). ¹H NMR (400 MHz, DMSO-d₆) δ 13.43-11.61 (broad m, 1H), 8.43 (s, 1H), 7.91 (s, 1H), 7.66 (s, 2H), 7.33-7.19 (m, 1H), 7.12 (s, 2H), 6.39 (s, 1H), 5.12 (d, J=9.6 Hz, 1H), 4.34 (t, J=11.4 Hz, 1H), 4.19 (s, 1H), 3.89 (p, J=8.8 Hz, 1H), 3.75-3.59 (br m, 1H), 2.68 (dt, J=10.2, 5.4 Hz, 2H), 2.28-1.78 (m, 9H), 1.69 (dd, J=15.2, 8.3 Hz, 1H), 1.39 (d, J=15.0 Hz, 1H), 1.05 (s, 6H), 0.50 (s, 9H).

### Example 115: Preparation of Compound 143

### Step 1: tert-butyl 6-[[(1R)-1-(hydroxymethyl)-3,3-dimethyl-butyl]amino]-3-azabicyclo[3.1.1]heptane-3-carboxylate

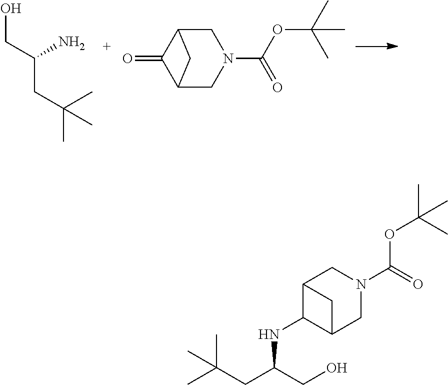

tert-butyl 6-oxo-3-azabicyclo[3.1.1]heptane-3-carboxylate (150 mg, 0.7100 mmol) was added to a solution of (2R)-2-amino-4,4-dimethyl-pentan-1-ol (hydrochloride salt) (146 mg, 0.8707 mmol) in anhydrous dichloromethane (1 mL) and stirred at room temperature for 30 minutes. sodium triacetoxyborohydride (480 mg, 2.265 mmol) was added and the reaction was stirred an additional 3 hours, then placed in a refrigerator for 16 hours. The reaction mixture was then allowed to warm to room temperature, diluted with 5 mL dichloromethane and aqueous HCl (3.04 mL of 1 M, 3.040 mmol) was added slowly and the reaction was stirred at room temperature an additional 10 minutes. A solution of potassium carbonate (1.25 g, 9.045 mmol) in water (1.25 mL) was added and the organics were separated. The aqueous was extracted with DCM then ethyl acetate, and the combined organics were washed with brine and dried over sodium sulfate to give as a colorless oil after concentration, tert-butyl 6-[[(1R)-1-(hydroxymethyl)-3,3-dimethyl-butyl]amino]-3-azabicyclo[3.1.1]heptane-3-carboxylate (190 mg, 82%). ESI-MS m/z calc. 326.25696, found 327.6 (M+1)⁺; Retention time: 0.45 minutes; LC method D.

### Step 2: 3-[[4-[(2R)-2-[(3-tert-Butoxycarbonyl-3-azabicyclo[3.1.1]heptan-6-yl)amino]-4,4-dimethyl-pentoxy]-6-(2,6-dimethylphenyl)pyrimidin-2-yl]sulfamoyl]benzoic acid

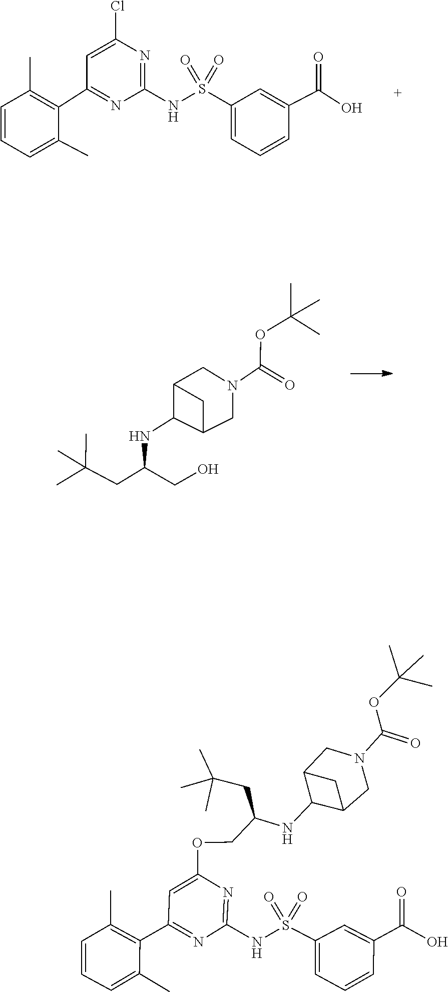 [SMILES: Cc1cccc(C)c1-c1cc(Cl)nc(NS(=O)(=O)c2cccc(C(=O)O)c2)n1 ; CC(C)(C)C[C@H](CO)NC1C2CC1CN(C(=O)OC(C)(C)C)C2 ; Cc1cccc(C)c1-c1cc(OC[C@@H](CC(C)(C)C)NC2C3CC2CN(C(=O)OC(C)(C)C)C3)nc(NS(=O)(=O)c2cccc(C(=O)O)c2)n1]

3-[[4-chloro-6-(2,6-dimethylphenyl)pyrimidin-2-yl]sulfamoyl]benzoic acid (240 mg, 0.5743 mmol) and tert-butyl 6-[[(1R)-1-(hydroxymethyl)-3,3-dimethyl-butyl]amino]-3-azabicyclo[3.1.1]heptane-3-carboxylate (190 mg, 0.5820 mmol) were combined in THE (1 mL) and sodium tert-butoxide (277 mg, 2.882 mmol) was added in two portions. The reaction was stirred one hour. The mixture was partitioned between ethyl acetate and 1M HCl. The organics were separated and the aqueous was extracted an additional 3×ethyl acetate. The combined organics were washed with brine, dried over sodium sulfate and evaporated. The resulting material was purified by reverse phase chromatography (1-99% MeOH in water, HCl modifier) to give as a white powder upon drying 3-[[4-[(2R)-2-[(3-tert-butoxycarbonyl-3-azabicyclo[3.1.1]heptan-6-yl)amino]-4,4-dimethyl-pentoxy]-6-(2,6-dimethylphenyl)pyrimidin-2-yl]sulfamoyl]benzoic acid (hydrochloride salt) (125 mg, 29%). ESI-MS m/z calc. 707.33527, found 708.6 (M+1)⁺; Retention time: 0.58 minutes; LC method D.

### Step 3: Methyl (1S,5R)-6-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]-3-azabicyclo[3.1.1]heptane-3-carboxylate (Compound 143)

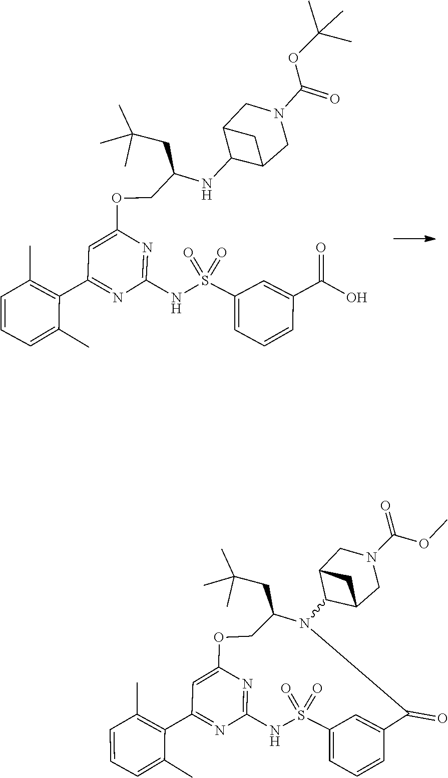 [SMILES: Cc1cccc(C)c1-c1cc(OC[C@@H](CC(C)(C)C)NC2C3CC2CN(C(=O)OC(C)(C)C)C3)nc(NS(=O)(=O)c2cccc(C(=O)O)c2)n1 ; COC(=O)N1C[C@H]2C[C@@H](C1)C2N1C(=O)c2cccc(c2)S(=O)(=O)Nc2nc(cc(-c3c(C)cccc3C)n2)OC[C@H]1CC(C)(C)C]

3-[[4-[(2R)-2-[(3-tert-Butoxycarbonyl-3-azabicyclo[3.1.1]heptan-6-yl)amino]-4,4-dimethyl-pentoxy]-6-(2,6-dimethylphenyl)pyrimidin-2-yl]sulfamoyl]benzoic acid (hydrochloride salt) (125 mg, 0.1679 mmol) in DMF (3 mL) was added dropwise to a stirring solution of COMU (150 mg, 0.3502 mmol) and DIPEA (150 μL, 0.8612 mmol) in DMF (17 mL). The reaction mixture was stirred at room temperature for 24 hours (at the 8 hour point additional COMU (75 mg, 0.1751 mmol) and DIPEA (50 μL, 0.2871 mmol) were added). Although the starting material appeared to show two stereoisomers, the cyclized product appeared to only go to a single peak. After the indicated reaction time, the reaction mixture was quenched with water and partially concentrated. The crude mixture was partitioned between 1M HCl and ethyl acetate, and the aqueous layer was extracted an additional 3×with ethyl acetate. The combined organics were washed with brine, dried over sodium sulfate and concentrated. The crude material was purified by chromatography on silica gel, eluting with a gradient of 0-100% ethyl acetate in hexanes to give tert-butyl (1S,5R)-6-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]-3-azabicyclo[3.1.1]heptane-3-carboxylate (20 mg, 17%) ESI-MS m/z calc. 689.3247, found 690.6 (M+1)⁺; Retention time: 0.81 minutes, LC method D.

The product was combined with HCl (850 μL of 4 M, 3.400 mmol) (in dioxane) and DCM (1 mL) and stirred for 30 minutes at room temperature. The reaction mixture was then concentrated, hexanes were added then evaporated, to give as a yellowish solid after drying (11R)-12-[(1S,5R)-3-azabicyclo[3.1.1]heptan-6-yl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (19 mg, 18%) ESI-MS m/z calc. 589.2723, found 590.5 (M+1)⁺; Retention time: 0.5 minutes. LC method D.

8 mg of the product from above were combined in DCM (0.5 mL) with methyl chloroformate (3 μL, 0.03883 mmol) and DIPEA (30 μL, 0.1722 mmol), and stirred for 5 minutes at room temperature. The reaction mixture was then quenched with several drops of 1M HCl, partially concentrated, then diluted with 1:1 DMSO/methanol, filtered, and purified by reverse phase HPLC (1-99% ACN in water, HCl modifier, 15 min run) to give as a white powder upon drying methyl (1S,5R)-6-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]-3-azabicyclo[3.1.1]heptane-3-carboxylate (4.7 mg, 4%) ESI-MS m/z calc. 647.2778, found 648.5 (M+1)⁺; Retention time: 1.75 minutes (LC method A).

### Example 116: Preparation of Compound 144

### Step 1: 3-[[4-[(2R)-2-[(6-tert-Butoxycarbonyl-6-azaspiro[3.5]nonan-2-yl)amino]-4,4-dimethyl-pentoxy]-6-(2,6-dimethylphenyl)pyrimidin-2-yl]sulfamoyl]benzoic acid

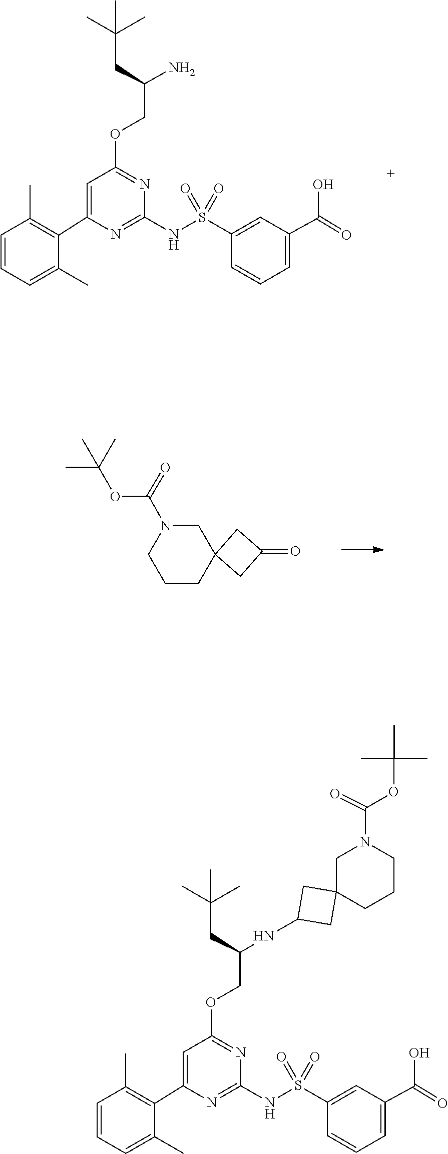 [SMILES: Cc1cccc(C)c1-c1cc(OC[C@H](N)CC(C)(C)C)nc(NS(=O)(=O)c2cccc(C(=O)O)c2)n1 ; CC(C)(C)OC(=O)N1CCCC2(CC(=O)C2)C1 ; Cc1cccc(C)c1-c1cc(OC[C@@H](CC(C)(C)C)NC2CC3(CCCN(C(=O)OC(C)(C)C)C3)C2)nc(NS(=O)(=O)c2cccc(C(=O)O)c2)n1]

In a reaction vial, 3-[[4-[(2R)-2-amino-4,4-dimethyl-pentoxy]-6-(2,6-dimethylphenyl)pyrimidin-2-yl]sulfamoyl]benzoic acid (hydrochloride salt) (193 mg, 0.3515 mmol) was mixed with tert-butyl 2-oxo-6-azaspiro[3.5]nonane-6-carboxylate (126.2 mg, 0.5273 mmol) in methylene chloride (920 μL). The reaction mixture was stirred at rt for 15 min then sodium triacetoxyborohydride (193.7 mg, 0.9139 mmol) was added. The reaction was stirred at rt for 4 h. then additional sodium triacetoxyborohydride (74.5 mg, 0.3515 mmol) was added. The reaction was stirred at rt for 12 h. then partitioned between ethyl acetate and 1N HCl. The reaction mixture was extracted with ethyl acetate (3×) and the organic layer was washed with saturated NaCl solution. The organic layer was isolated, dried over anhydrous sodium sulfate, filtered, and evaporated to dryness. The crude material was purified by reverse phase HPLC using 20-80% ACN/water gradient to provide the product as a white solid. The final product was collected as a mixture of diastereomers then partitioned between ethyl acetate and 1N HCl. The reaction mixture was extracted with ethyl acetate (3×) and the organic layer was washed with saturated NaCl solution. The organic layer was isolated, dried over anhydrous sodium sulfate, filtered, and evaporated to dryness. The crude material was purified by reverse phase HPLC using 20-80% ACN/water gradient to provide the product as a white solid. The final product was collected as a mixture of diastereomers 3-[[4-[(2R)-2-[(6-tert-butoxycarbonyl-6-azaspiro[3.5]nonan-2-yl)amino]-4,4-dimethyl-pentoxy]-6-(2,6-dimethylphenyl)pyrimidin-2-yl]sulfamoyl]benzoic acid (hydrochloride salt) (80 mg, 29%) ESI-MS m/z calc. 735.3666, found 736.4 (M+1)⁺; Retention time: 0.57 (LC method D).

### Step 2: tert-butyl 2-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]-6-azaspiro[3.5]nonane-6-carboxylate, diastereomer 1, and tert-butyl 2-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]-6-azaspiro[3.5]nonane-6-carboxylate, diastereomer 2

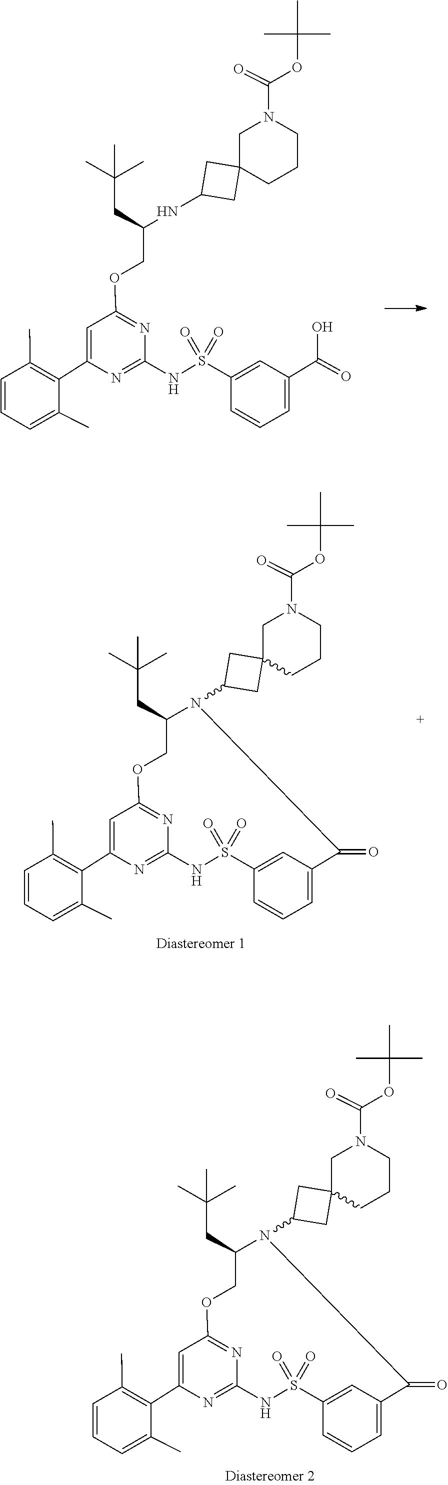 [SMILES: Cc1cccc(C)c1-c1cc(OC[C@@H](CC(C)(C)C)NC2CC3(CCCN(C(=O)OC(C)(C)C)C3)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CCCN(C(=O)OC(C)(C)C)C3)C1)[C@H](CC(C)(C)C)CO2 ; Cc1cccc(C)c1-c1cc2nc(n1)NS(=O)(=O)c1cccc(c1)C(=O)N(C1CC3(CCCN(C(=O)OC(C)(C)C)C3)C1)[C@H](CC(C)(C)C)CO2]

In a reaction vial, 3-[[4-[(2R)-2-[(6-tert-butoxycarbonyl-6-azaspiro[3.5]nonan-2-yl)amino]-4,4-dimethyl-pentoxy]-6-(2,6-dimethylphenyl)pyrimidin-2-yl]sulfamoyl]benzoic acid (hydrochloride salt) (80 mg, 0.1025 mmol) was dissolved in DMF (4 mL) along with 4-methylmorpholine (22.6 μL, 0.2056 mmol) and cooled to 0° C. To the reaction, 2-chloro-4,6-dimethoxy-1,3,5-triazine (18 mg, 0.1025 mmol) was added and the reaction was allowed to stir at 0° C. for 1 h. then additional 4-methylmorpholine (11.3 μL, 0.1028 mmol) was added. The reaction was allowed to warm to rt and stirred at that temperature overnight. The reaction was concentrated to a third of the volume then partitioned between ethyl acetate and 1N HCl solution. The organics were separated, dried over sodium sulfate, and evaporated to dryness. The crude material was purified by column chromatography on silica using 10-60% ethyl acetate/hexanes gradient to give two isomers: First to elute, diastereomer 1, tert-butyl 2-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]-6-azaspiro[3.5]nonane-6-carboxylate (15.6 mg, 21%) ESI-MS m/z calc. 717.356, found 718.5 (M+1)⁺; Retention time: 2.24 minutes (LC method A); and second to elute, diastereomer 2, tert-butyl 2-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]-6-azaspiro[3.5]nonane-6-carboxylate (18.8 mg, 25%) ESI-MS m/z calc. 717.356, found 718.4 (M+1)⁺; Retention time: 2.28 minutes (LC method A).

### Step 3: (11R)-6-(2,6-Dimethylphenyl)-11-(2,2-dimethylpropyl)-12-[6-(2-hydroxyacetyl)-6-azaspiro[3.5]nonan-2-yl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 144)

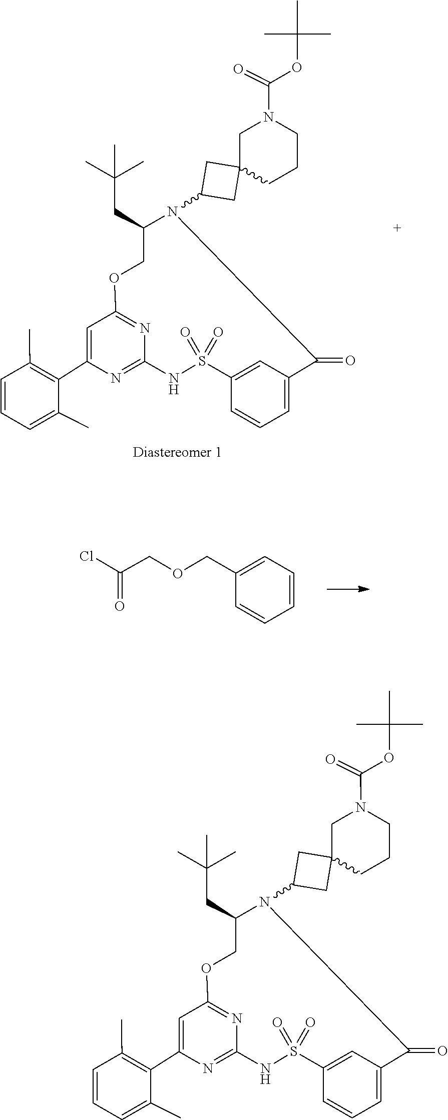 [SMILES: Cc1cccc(C)c1-c1cc2nc(n1)NS(=O)(=O)c1cccc(c1)C(=O)N(C1CC3(CCCN(C(=O)OC(C)(C)C)C3)C1)[C@H](CC(C)(C)C)CO2 ; O=C(Cl)COCc1ccccc1 ; Cc1cccc(C)c1-c1cc2nc(n1)NS(=O)(=O)c1cccc(c1)C(=O)N(C1CC3(CCCN(C(=O)OC(C)(C)C)C3)C1)[C@H](CC(C)(C)C)CO2]

In a reaction vial, tert-butyl 2-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]-6-azaspiro[3.5]nonane-6-carboxylate (diastereomer 1, 15.6 mg, 0.02173 mmol) was dissolved in (1:1) trifluoroacetic acid (250 μL, 3.245 mmol)/methylene chloride (250 μL) and stirred at rt for 1 h The reaction mixture was evaporated to dryness. The material was precipitated from ethyl acetate/hexanes to provide a white solid. The material was used for the next reaction without further purification. In a reaction vial, (11R)-12-(6-azaspiro[3.5]nonan-2-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (trifluoroacetate salt) (18.6 mg, 117%) was dissolved in methylene chloride (500 μL) along with diisopropylethylamine (25 μL, 0.1435 mmol) and cooled to −10° C. To the reaction mixture, 2-benzyloxyacetyl chloride (11.32 μL, 0.07174 mmol) was added and the reaction was allowed to warm to rt After stirring at rt, the reaction was partitioned between ethyl acetate and 1N HCl. The organic layer was washed with saturated NaCl solution, dried over anhydrous sodium sulfate, filtered, and evaporated to dryness. The crude material was dissolved in methanol (1 mL) along with a few drops of water. The reaction mixture was flushed with nitrogen (3×) then palladium (50 mg, 0.4698 mmol) was added and the reaction was stirred at rt under hydrogen atmosphere for 1 h. then filtered through Celite. The filtrate was evaporated to dryness and the crude material was purified by preparative HPLC. The product was recovered as a white solid. (11R)-6-(2,6-dimethylphenyl)-11-(2,2-dimethylpropyl)-12-[6-(2-hydroxyacetyl)-6-azaspiro[3.5]nonan-2-yl]-2,2-dioxo-9-oxa-2λ⁶-thia-3,5,12,19-tetrazatricyclo[12.3.1.14,8]nonadeca-1(18),4(19),5,7,14,16-hexaen-13-one (6.1 mg, 42%) ESI-MS m/z calc. 675.3091, found 676.3 (M+1)⁺; Retention time: 1.66 minutes (LC method A).

### Example 117: Preparation of Compound 145

### Step 3: (11R)-6-(2,6-Dimethylphenyl)-11-(2,2-dimethylpropyl)-12-[6-(2-hydroxyacetyl)-6-azaspiro[3.5]nonan-2-yl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 145)

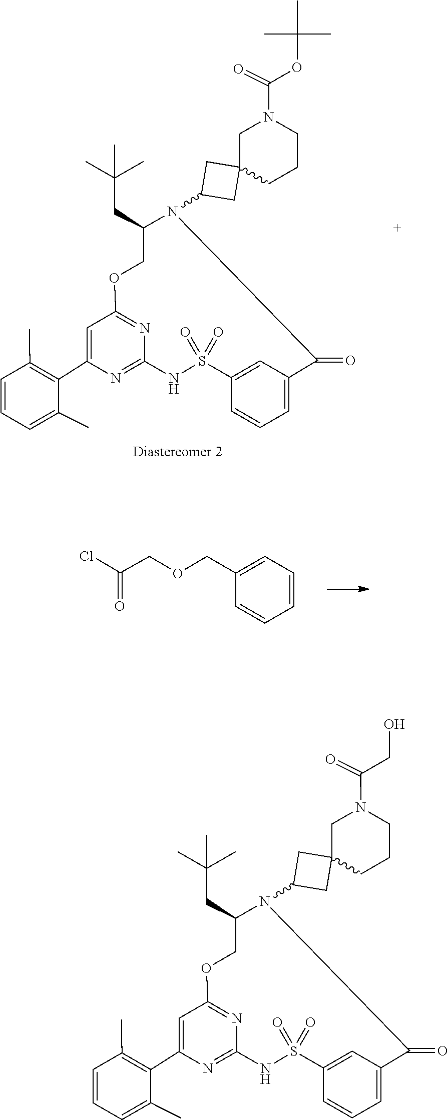 [SMILES: Cc1cccc(C)c1-c1cc2nc(n1)NS(=O)(=O)c1cccc(c1)C(=O)N(C1CC3(CCCN(C(=O)OC(C)(C)C)C3)C1)[C@H](CC(C)(C)C)CO2 ; O=C(Cl)COCc1ccccc1 ; Cc1cccc(C)c1-c1cc2nc(n1)NS(=O)(=O)c1cccc(c1)C(=O)N(C1CC3(CCCN(C(=O)CO)C3)C1)[C@H](CC(C)(C)C)CO2]

In a reaction vial, tert-butyl 2-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]-6-azaspiro[3.5]nonane-6-carboxylate (diastereomer 2, 18.8 mg, 0.02593 mmol) was dissolved in (1:1) trifluoroacetic acid (250 μL, 3.245 mmol)/dichloromethane (250 μL) and stirred at rt for 1 h. The reaction mixture was evaporated to dryness. The material was precipitated from ethyl acetate/hexanes to provide a white solid. The material was used for the next reaction without further purification. In a reaction vial, (11R)-12-(6-azaspiro[3.5]nonan-2-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (trifluoroacetate salt) (26.9 mg, 142%) was dissolved in methylene chloride (500 μL) along with diisopropylethylamine (29.8 μL, 0.1711 mmol) and cooled to −10° C. To the reaction mixture, 2-benzyloxyacetyl chloride (13.5 μL, 0.08555 mmol) was added and the reaction was allowed to warm to rt After stirring at rt for x h, the reaction was partitioned between ethyl acetate and 1N HCl. The organic layer was washed with saturated NaCl solution, dried over anhydrous sodium sulfate, filtered, and evaporated to dryness. The crude material was dissolved in methanol (1 mL) along with a few drops of water. The reaction mixture was flushed with nitrogen (3×) then palladium (50 mg, 0.4698 mmol) was added and the reaction was stirred at rt under hydrogen atmosphere for 1 h. then filtered through Celite. The filtrate was evaporated to dryness and the crude material was purified by preparative HPLC. The product was recovered as a white solid. (11R)-6-(2,6-dimethylphenyl)-11-(2,2-dimethylpropyl)-12-[6-(2-hydroxyacetyl)-6-azaspiro[3.5]nonan-2-yl]-2,2-dioxo-9-oxa-2λ⁶-thia-3,5,12,19-tetrazatricyclo[12.3.1.14,8]nonadeca-1(18),4(19),5,7,14,16-hexaen-13-one (8.6 mg, 49%) ESI-MS m/z calc. 675.3091, found 676.3 (M+1)⁺; Retention time: 1.79 minutes (LC method A).

### Example 118: Preparation of Compound 146

### Step 1: 3-[[4-[(2R)-2-[(1-tert-Butoxycarbonyl-1-azaspiro[3.3]heptan-6-yl)amino]-4,4-dimethyl-pentoxy]-6-(2,6-dimethylphenyl)pyrimidin-2-yl]sulfamoyl]benzoic acid

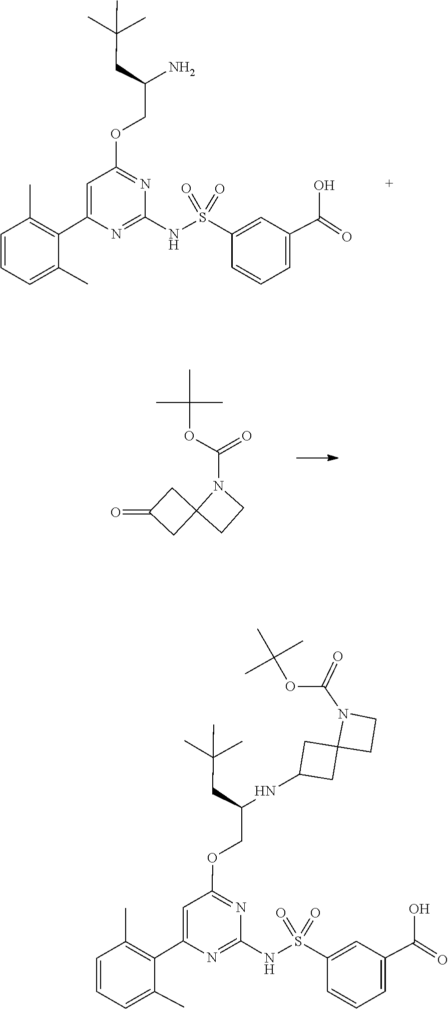 [SMILES: Cc1cccc(C)c1-c1cc(OC[C@H](N)CC(C)(C)C)nc(NS(=O)(=O)c2cccc(C(=O)O)c2)n1 ; CC(C)(C)OC(=O)N1CCC12CC(=O)C2 ; Cc1cccc(C)c1-c1cc(OC[C@@H](CC(C)(C)C)NC2CC3(CCN3C(=O)OC(C)(C)C)C2)nc(NS(=O)(=O)c2cccc(C(=O)O)c2)n1]

In a reaction vial, 3-[[4-[(2R)-2-amino-4,4-dimethyl-pentoxy]-6-(2,6-dimethylphenyl)pyrimidin-2-yl]sulfamoyl]benzoic acid (hydrochloride salt) (800 mg, 1.457 mmol) was mixed with tert-butyl 6-oxo-1-azaspiro[3.3]heptane-1-carboxylate (308 mg, 1.458 mmol) in methylene chloride (5.2 mL). The reaction mixture was stirred at rt for 15 min then sodium triacetoxyborohydride (618 mg, 2.916 mmol) was added. The reaction was stirred at rt for 1.5 h. then partitioned between ethyl acetate and 1N HCl. The reaction mixture was extracted with ethyl acetate (3×) and the organic layer was washed with saturated NaCl solution. The organic layer was isolated, dried over anhydrous sodium sulfate, filtered, and evaporated to dryness. The crude material was purified by reverse phase chromatography using 20-100% water/ACN gradient. The product fractions were made basic by addition of triethylamine then evaporated to dryness. The residue was partitioned between ethyl acetate and 1N HCl. The aqueous layer was extracted with ethyl acetate. The combined organic layer was washed with saturated NaCl solution, dried over anhydrous sodium sulfate, filtered, and evaporated to dryness to give two products as white solids: 3-[[4-[(2R)-2-[(1-tert-butoxycarbonyl-1-azaspiro[3.3]heptan-6-yl)amino]-4,4-dimethyl-pentoxy]-6-(2,6-dimethylphenyl)pyrimidin-2-yl]sulfamoyl]benzoic acid (hydrochloride salt) (108 mg, 10%)¹H NMR (400 MHz, DMSO-d₆) δ 9.11 (s, 1H), 8.47 (t, J=1.8 Hz, 1H), 8.14 (t, J=7.8 Hz, 2H), 7.69 (t, J=7.8 Hz, 1H), 7.26 (t, J=7.6 Hz, 1H), 7.13 (d, J=7.6 Hz, 2H), 6.32 (s, 1H), 5.02 (s, 1H), 4.42-4.18 (m, 1H), 4.23-4.03 (m, 1H), 3.87-3.77 (m, 1H), 3.71-3.60 (m, 3H), 2.36-2.18 (m, 5H), 2.19-2.08 (m, 3H), 1.40 (s, 7H), 1.35 (s, 9H), 0.93 (s, 9H). ESI-MS m/z calc. 707.33527, found 708.6 (M+1)⁺; Retention time: 1.93 minutes (LC method A); and 3-[[4-[(2R)-2-[[3-[2-(tert-butoxycarbonylamino)ethyl]cyclobutyl]amino]-4,4-dimethyl-pentoxy]-6-(2,6-dimethylphenyl)pyrimidin-2-yl]sulfamoyl]benzoic acid (hydrochloride salt) (315.4 mg, 28%)¹H NMR (400 MHz, DMSO-d₆) δ 8.52-8.43 (m, 1H), 8.14 (ddd, J=9.2, 5.2, 1.6 Hz, 2H), 7.70 (t, J=7.8 Hz, 1H), 7.27 (t, J=7.6 Hz, 1H), 7.13 (d, J=7.6 Hz, 2H), 6.78 (q, J=5.3 Hz, 1H), 6.3 5 (s, 1H), 4.31 (d, J=12.6 Hz, 1H), 4.21-4.07 (m, 1H), 4.03 (q, J=7.1 Hz, 1H), 3.95-3.80 (m, 1H), 3.75-3.55 (m, 1H), 2.96-2.77 (m, 2H), 2.41-2.29 (m, 1H), 2.27-2.17 (m, 1H), 2.17-2.07 (m, 1H), 2.00 (d, J=12.4 Hz, 6H), 1.73-1.60 (m, 1H), 1.63-1.51 (m, 2H), 1.50-1.39 (m, 2H), 1.36 (s, 9H), 0.93 (s, 9H). ESI-MS m/z calc. 709.3509, found 610.5 (M+1-Boc)⁺; Retention time: 1.4 minutes (LC method A).

### Step 2: (11R)-12-(1-azaspiro[3.3]heptan-6-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

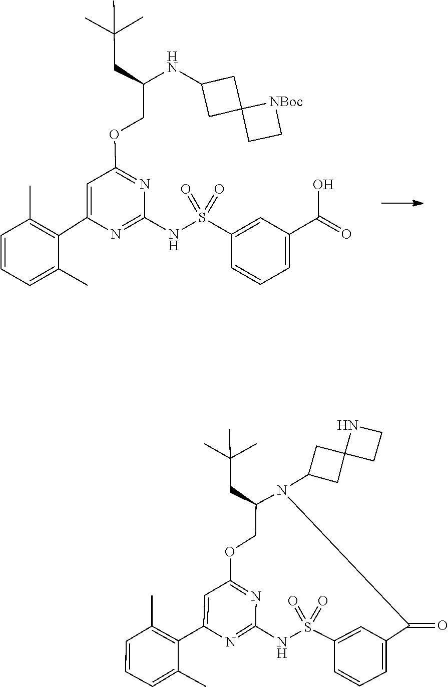 [SMILES: Cc1cccc(C)c1-c1cc(OC[C@@H](CC(C)(C)C)NC2CC3(CCN3C(=O)OC(C)(C)C)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CCN3)C1)[C@H](CC(C)(C)C)CO2]

In a reaction via, 3-[[4-[(2R)-2-[(1-tert-butoxycarbonyl -1-azaspiro[3.3]heptan-6-yl)amino]-4,4-dimethyl-pentoxy]-6-(2,6-dimethylphenyl)pyrimidin-2-yl]sulfamoyl]benzoic acid (hydrochloride salt) (106 mg, 0.1396 mmol) was dissolved in DMF (4.9 mL) along with 4-methylmorpholine (31 μL, 0.2820 mmol) and cooled to 0° C. To the reaction, 2-chloro-4,6-dimethoxy-1,3,5-triazine (24.6 mg, 0.1401 mmol) was added and the reaction was allowed to stir at 0° C. for 1.5 h. then additional 4-methylmorpholine (15.5 μL, 0.1410 mmol) was added. The reaction was allowed to warm to rt and stirred at that temperature overnight. The reaction was concentrated to a third of the volume then partitioned between ethyl acetate and 0.5 N HCl solution. The organics were separated, dried over sodium sulfate, and evaporated to dryness. The crude material was purified by column chromatography on silica using 20-100% ethyl acetate/hexanes gradient. The product was isolated as a white solid. (11R)-12-(1-Azaspiro[3.3]heptan-6-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (45.5 mg, 55%). ¹H NMR (400 MHz, Chloroform-d) δ 8.67 (t, J=1.9 Hz, 1H), 7.94 (d, J=7.8 Hz, 1H), 7.85 (d, J=7.6 Hz, 1H), 7.59 (t, J=7.8 Hz, 1H), 7.21 (t, J=7.6 Hz, 1H), 7.06 (d, J=7.6 Hz, 2H), 6.18 (s, 1H), 5.42 (dd, J=10.6, 4.2 Hz, 1H), 4.18-4.01 (m, 1H), 3.91-3.77 (m, 4H), 3.03 (s, 2H), 2.84-2.78 (m, 1H), 2.47 (dt, J=10.4, 6.4 Hz, 1H), 2.41-2.31 (m, 3H), 1.98 (s, 6H), 1.71 (dd, J=15.1, 8.3 Hz, 2H), 1.31-1.20 (m, 1H), 0.58 (s, 9H). ESI-MS m/z calc. 589.2723, found 590.3 (M+1)⁺; Retention time: 2.04 minutes (LC method A).

### Step 3: (11R)-6-(2,6-Dimethylphenyl)-11-(2,2-dimethylpropyl)-12-[1-(2-hydroxyacetyl)-1-azaspiro[3.3]heptan-6-yl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 146)

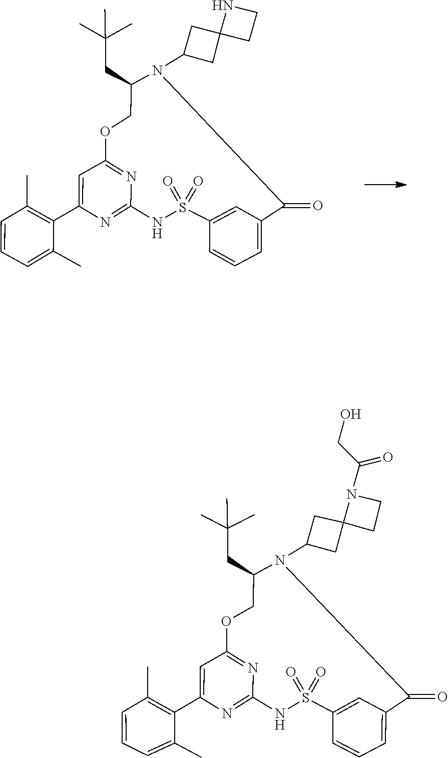 [SMILES: Cc1cccc(C)c1-c1cc2nc(n1)NS(=O)(=O)c1cccc(c1)C(=O)N(C1CC3(CCN3)C1)[C@H](CC(C)(C)C)CO2 ; Cc1cccc(C)c1-c1cc2nc(n1)NS(=O)(=O)c1cccc(c1)C(=O)N(C1CC3(CCN3C(=O)CO)C1)[C@H](CC(C)(C)C)CO2]

In a reaction vial, tert-butyl 6-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]-1-azaspiro[3.3]heptane-1-carboxylate (43 mg, 0.07291 mmol) was mixed with diisopropylethylamine (37.5 μL, 0.2153 mmol) in methylene chloride (900 μL). The reaction was cooled to −10° C. then 2-benzyloxyacetyl chloride (11.3 μL, 0.07161 mmol) was added. The reaction was allowed to warm to 0° C. After stirring at 0° C. for 30 min, the reaction mixture was evaporated to dryness then diluted with ethyl acetate then washed with 1N HCl (3×) and sat. NaCl solution. The organic layer was isolated, dried over sodium sulfate, filtered, and evaporated to dryness. The crude material was used for the next step without further purification. In a reaction vial, (11R)-12-[1-(2-benzyloxyacetyl)-1-azaspiro[3.3]heptan-6-yl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (49.3 mg, 92%) was dissolved in Methanol (1.5 mL) along with 1 drop of water. The reaction mixture was flushed with nitrogen then palladium (60 mg of 10% w/w, 0.05638 mmol) was added. The reaction was stirred under hydrogen atmosphere for 1 hr. The reaction was filtered through Celite and evaporated to dryness. The crude material was purified by column chromatography on silica using 0-10% MeOH/DCM gradient. The isolated product was further purified by preparative HPLC to provide the product as an off-white solid. (11R)-6-(2,6-Dimethylphenyl)-11-(2,2-dimethylpropyl)-12-[1-(2-hydroxyacetyl)-1-azaspiro[3.3]heptan-6-yl]-2,2-dioxo-9-oxa-2λ⁶-thia-3,5,12,19-tetrazatricyclo[12.3.1.14,8]nonadeca-1(18),4(19),5,7,14,16-hexaen-13-one (13.1 mg, 25%). ¹H NMR (400 MHz, Methanol-d₄) δ 8.63 (d, J=37.3 Hz, 1H), 8.03 (dt, J=7.6, 1.5 Hz, 1H), 7.71 (dt, J=19.2, 7.5 Hz, 2H), 7.27 (t, J=7.6 Hz, 1H), 7.13 (d, J=7.7 Hz, 2H), 6.26 (d, J=12.6 Hz, 1H), 4.60-4.43 (m, 2H), 4.11 (t, J=7.6 Hz, 1H), 3.99 (s, 2H), 3.92-3.84 (m, 2H), 3.83-3.74 (m, 1H), 2.67-2.44 (m, 4H), 2.20 (s, 2H), 1.55 (d, J=15.1 Hz, 2H), 1.37 (s, 2H), 1.29 (s, 3H), 0.94-0.79 (m, 3H), 0.60 (d, J=3.2 Hz, 9H). ESI-MS m/z calc. 647.2778, found 648.3 (M+1)⁺(LC method A).

### Example 119: Preparation of Compound 147, Compound 148, and Compound 149

### Step 1: (11R)-12-(2-Cyclopropylcyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound 147), (11R)-12-(2-Cyclopropylcyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 2 (Compound 148) and (11R)-12-(2-Cyclopropylcyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one, mixture of diastereomer 3 and 4 (Compound 149)

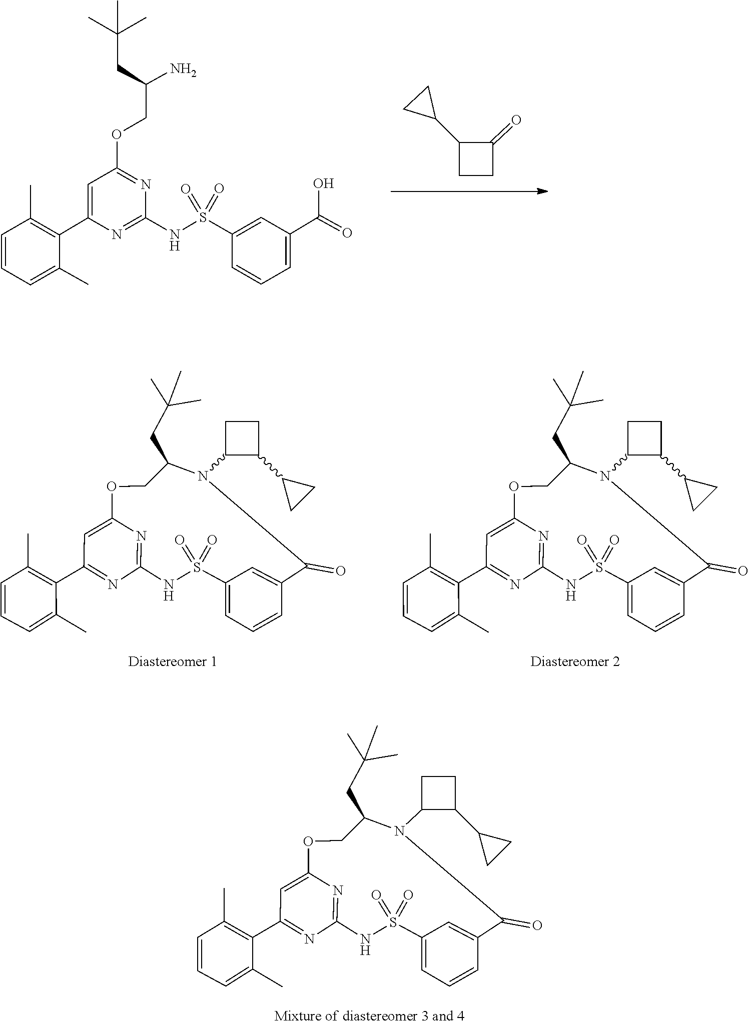 [SMILES: Cc1cccc(C)c1-c1cc(OC[C@H](N)CC(C)(C)C)nc(NS(=O)(=O)c2cccc(C(=O)O)c2)n1 ; O=C1CCC1C1CC1 ; Cc1cccc(C)c1-c1cc2nc(n1)NS(=O)(=O)c1cccc(c1)C(=O)N(C1CCC1C1CC1)[C@H](CC(C)(C)C)CO2 ; Cc1cccc(C)c1-c1cc2nc(n1)NS(=O)(=O)c1cccc(c1)C(=O)N(C1CCC1C1CC1)[C@H](CC(C)(C)C)CO2 ; Cc1cccc(C)c1-c1cc2nc(n1)NS(=O)(=O)c1cccc(c1)C(=O)N(C1CCC1C1CC1)[C@H](CC(C)(C)C)CO2]

3-[[4-[(2R)-2-amino-4,4-dimethyl-pentoxy]-6-(2,6-dimethylphenyl)pyrimidin-2-yl]sulfamoyl]benzoic acid (hydrochloride salt) (70 mg, 0.1275 mmol) was combined with 2-cyclopropylcyclobutanone (18 mg, 0.1634 mmol) and stirred at room temperature for 15 minutes in DCM (0.5 mL). Sodium triacetoxyborohydride was added and the reaction was stirred for an additional 20 minutes. Additional sodium triacetoxyborohydride (80 mg, 0.3775 mmol) was added, and the reaction was stirred for an additional 4 hours at room temperature. The reaction mixture was then partitioned between 1M HCl and ethyl acetate. The layers were separated and the aqueous was extracted an additional 3×ethyl acetate. The combined organics were washed with brine, dried over sodium sulfate and concentrated. The resulting crude material was dissolved in 1:1 DMSO/methanol, filtered, and purified by reverse phase HPLC (1-70% ACN in water, HCl modifier, 15 min run) to give the reductive amination product as a white solid.

The product from above was combined with CDMT (25 mg, 0.1424 mmol) in DMF (10 mL) and N-methylmorpholine (45 μL, 0.4093 mmol) was added via syringe. After stirring for the indicated time at room temperature the reaction mixture was concentrated to a volume of less than 1 mL by rotary evaporation, filtered, and purified by reverse phase HPLC (1-99% ACN in water, HCl modifier, 30 min run) to give 4 stereoisomers as products, which were isolated as single stereoisomers and two of which were isolated together as a diastereomeric pair: Diastereomer 1, (11R)-12-(2-cyclopropylcyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (1.5 mg, 2%), ESI-MS m/z calc. 588.27704, found 589.5 (M+1)⁺; Retention time: 2.06 minutes; LC method A; and diastereomer 2, (11R)-12-(2-cyclopropylcyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (1.5 mg, 2%), ESI-MS m/z calc. 588.27704, found 589.5 (M+1)⁺; Retention time: 2.1 minutes; LC method A; and a mixture of diastereomer 3 and 4, (11R)-12-(2-cyclopropylcyclobutyl)-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione, ESI-MS m/z calc. 588.27704, found 589.5 (M+1)⁺; Retention time: 2.13 minutes; LC method A.

### Example 120: Preparation of Compound 150 and Compound 151

### Step 1: (11R)-6-(2,6-Dimethylphenyl)-11-(2,2-dimethylpropyl)-12-(3-methoxycyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound 150), and (11R)-6-(2,6-dimethylphenyl)-11-(2,2-dimethylpropyl)-12-(3-methoxycyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 2 (Compound 151)

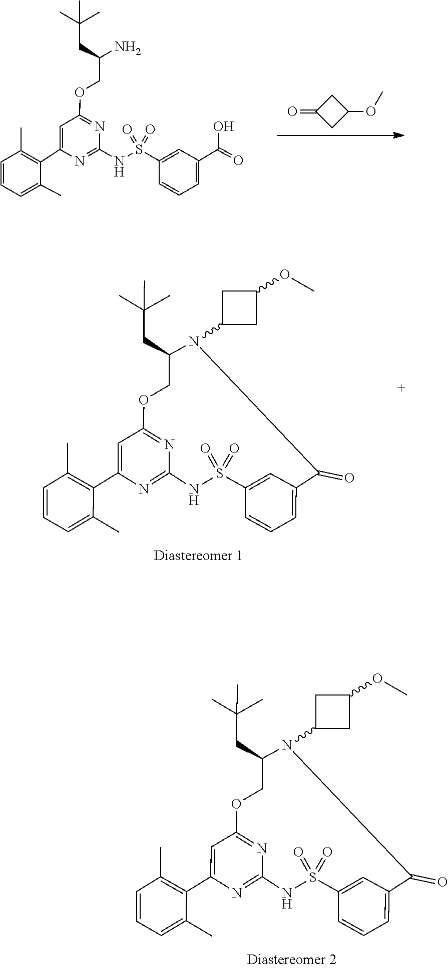

3-[[4-[(2R)-2-amino-4,4-dimethyl-pentoxy]-6-(2,6-dimethylphenyl)pyrimidin-2-yl]sulfamoyl]benzoic acid (hydrochloride salt) (75 mg, 0.1207 mmol) and 3-methoxycyclobutanone (18 mg, 0.1798 mmol) were combined and stirred in dichloromethane (3 mL) for 5 minutes as a suspension. One portion of sodium triacetoxyborohydride (Sodium salt) (77 mg, 0.3633 mmol) was added. A translucent-clear reaction mixture was obtained. After stirring at room temperature for 1 hour, another portion of sodium triacetoxyborohydride (Sodium salt) (40 mg, 0.1887 mmol) was added. The reaction mixture was allowed to stir overnight at room temperature. The reaction mixture was then diluted with EtOAc (10 mL) and washed with aqueous 1 M HCl (1×10 mL) and brine (1×10 mL). The organic layer was dried over sodium sulfate, filtered and concentrated under reduced pressure. HATU (92 mg, 0.2420 mmol) was dissolved in DMF (6 mL), and DIEA (100 μL, 0.5741 mmol) was added afterwards. This clear yellow solution was added to the crude intermediate obtained above. The reaction mixture was allowed to stir at room temperature overnight. The crude material was purified by reverse phase HPLC using a Luna C₁₈ (2) column (50×21.2 mm, 5 μm particle size) sold by Phenomenex (pn: 00B-4252-P0-AX), and a dual gradient run from 10-99% mobile phase B over 15.0 minutes. Mobile phase A=water (5 mM HCl acid modifier). Mobile phase B=acetonitrile. Flow rate=35 mL/min, injection volume=950 μL, and column temperature=25° C. The UV trace at 254 nm was used to collect fractions and two isomers were isolated: diastereomer 1, (11R)-6-(2,6-dimethylphenyl)-11-(2,2-dimethylpropyl)-12-(3-methoxycyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one (5.9 mg, 8%) was obtained. ESI-MS m/z calc. 578.2563, found 579.3 (M+1)⁺; Retention time: 1.84 minutes (LC method A); and diastereomer 2, (11R)-6-(2,6-dimethylphenyl)-11-(2,2-dimethylpropyl)-12-(3-methoxycyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one (4.8 mg, 7%) was obtained. ESI-MS m/z calc. 578.2563, found 579.3 (M+1)⁺; Retention time: 1.85 minutes. (LC method A).

### Example 121: Preparation of Compound 152 and Compound 153

### Step 1: 3-[[4-(2,6-Dimethylphenyl)-6-[(2R)-2-[[3-(2-methoxy-2-oxo-ethyl)cyclobutyl]amino]-4,4-dimethyl-pentoxy]pyrimidin-2-yl]sulfamoyl]benzoic acid

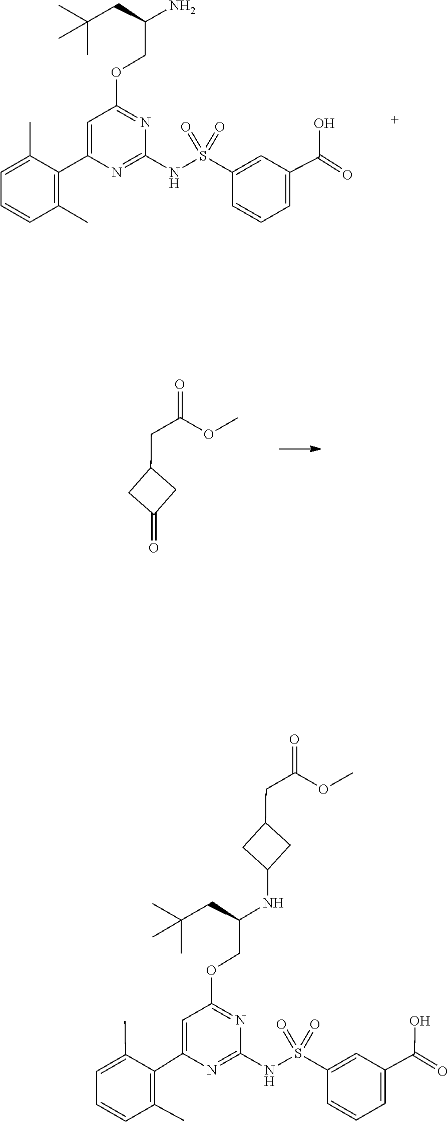

3-[[4-[(2R)-2-amino-4,4-dimethyl-pentoxy]-6-(2,6-dimethylphenyl)pyrimidin-2-yl]sulfamoyl]benzoic acid (hydrochloride salt) (274.54 mg, 0.5 mmol) was dissolved in DCM (4 mL) and methyl 2-(3-oxocyclobutyl)acetate (142.15 mg, 1.0000 mmol) was added. The mixture was stirred for 0.5 h, then NaBH(OAc)₃ (317.91 mg, 1.5000 mmol) was added, and the reaction was stirred for 3 d. The mixture was directly purified by silica gel chromatography (24 g, eluent DCM-MeOH 100:0 to 92:8 gradient) to afford 3-[[4-(2,6-dimethylphenyl)-6-[(2R)-2-[[3-(2-methoxy-2-oxo-ethyl)cyclobutyl]amino]-4,4-dimethyl-pentoxy]pyrimidin-2-yl]sulfamoyl]benzoic acid (165 mg, 51%) ESI-MS m/z calc. 638.2774, found 639.6 (M+H)⁺; Retention time: 2.62 minutes (LC method T).

### Step 2: Methyl 2-[3-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]acetate

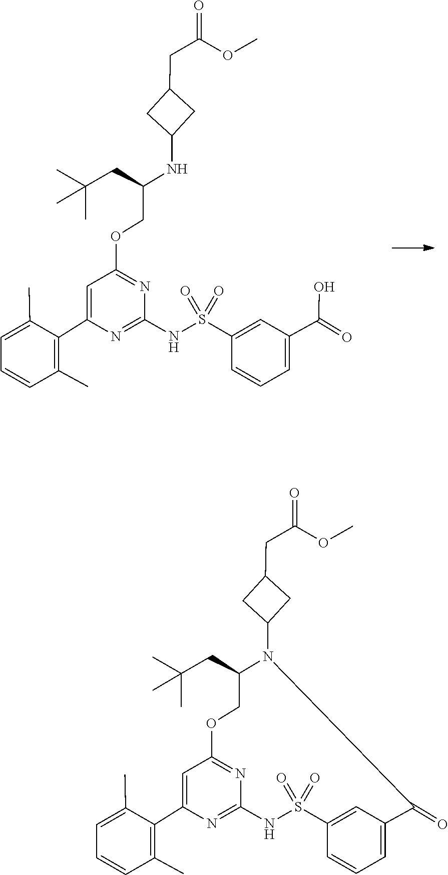 [SMILES: COC(=O)CC1CC(N[C@@H](COc2cc(-c3c(C)cccc3C)nc(NS(=O)(=O)c3cccc(C(=O)O)c3)n2)CC(C)(C)C)C1 ; COC(=O)CC1CC(N2C(=O)c3cccc(c3)S(=O)(=O)Nc3nc(cc(-c4c(C)cccc4C)n3)OC[C@H]2CC(C)(C)C)C1]

3-[[4-(2,6-dimethylphenyl)-6-[(2R)-2-[[3-(2-methoxy-2-oxo-ethyl)cyclobutyl]amino]-4,4-dimethyl-pentoxy]pyrimidin-2-yl]sulfamoyl]benzoic acid (150 mg, 0.2348 mmol) and COMU (121 mg, 0.2825 mmol) were dissolved in anhydrous DMF (5 mL), then DIPEA (148.40 mg, 0.2 mL, 1.1482 mmol) was added, and the solution stirred for 18 h. The mixture was diluted with EtOAc (25 mL), extracted with HCl (1M, 2×5 mL), then with saturated aqueous sodium bicarbonate (5 mL) and brine (5 mL). The organic phase was dried over sodium sulfate, filtered, concentrated, and purified by silica gel chromatography, eluent DCM-MeOH 100:0 to 90:10 gradient to afford methyl 2-[3-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]acetate (121 mg, 79%) as a white solid ESI-MS m/z calc. 620.2669, found 621.7 (M+H)⁺; Retention time: 3.52 minutes, LC method T.

### Step 3: (11R)-6-(2,6-Dimethylphenyl)-11-(2,2-dimethylpropyl)-12-[3-(2-hydroxy-2-methyl-propyl)cyclobutyl]-2,2-dioxo-9-oxa-2λ⁶-thia-3,5,12,19-tetrazatricyclo[12.3.1.14,8]nonadeca-1(18),4(19),5,7,14,16-hexaen-13-one

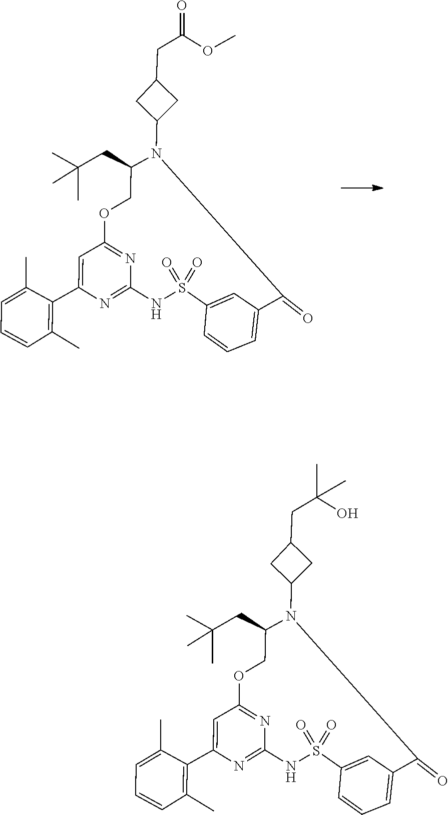 [SMILES: COC(=O)CC1CC(N2C(=O)c3cccc(c3)S(=O)(=O)Nc3nc(cc(-c4c(C)cccc4C)n3)OC[C@H]2CC(C)(C)C)C1 ; Cc1cccc(C)c1-c1cc2nc(n1)NS(=O)(=O)c1cccc(c1)C(=O)N(C1CC(CC(C)(C)O)C1)[C@H](CC(C)(C)C)CO2]

To a stirred solution of methyl 2-[3-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]acetate (50 mg, 0.0805 mmol) in anhydrous THE (2 mL), MeMgBr in THF:toluene (1:3) (0.5750 mL of 1.4 M, 0.8050 mmol) was added, and the resulting solution was stirred for 1 h. The mixture was quenched with saturated aqueous ammonium chloride (1 mL), extracted with EtOAc (2×3 mL), dried over sodium sulfate, concentrated, and purified by preparative HPLC to afford (11R)-6-(2,6-dimethylphenyl)-11-(2,2-dimethylpropyl)-12-[3-(2-hydroxy-2-methyl-propyl)cyclobutyl]-2,2-dioxo-9-oxa-2λ⁶-thia-3,5,12,19-tetrazatricyclo[12.3.1.14,8]nonadeca-1(18),4(19),5,7,14,16-hexaen-13-one (21.8 mg, 42%) ESI-MS m/z calc. 620.3032, found 621.5 (M+H)⁺; Retention time: 2.57 minutes, LC method W.

### Step 4: (11R)-6-(2,6-Dimethylphenyl)-11-(2,2-dimethylpropyl)-12-[3-(2-hydroxy-2-methylpropyl)cyclobutyl]-9-oxa-2λ⁶-thia-3,5,12,19-tetraazatricyclo[12.3.1.14,8]nonadeca-1(17),4(19),5,7,14(18),15-hexaene-2,2,13-trione, diastereomer 1 (Compound 152), and (11R)-6-(2,6-dimethylphenyl)-11-(2,2-dimethylpropyl)-12-[3-(2-hydroxy-2-methylpropyl)cyclobutyl]-9-oxa-2λ⁶-thia-3,5,12,19-tetraazatricyclo[12.3.1.14,8]nonadeca-1(17),4(19),5,7,14(18),15-hexaene-2,2,13-trione, diastereomer 2 (Compound 153)

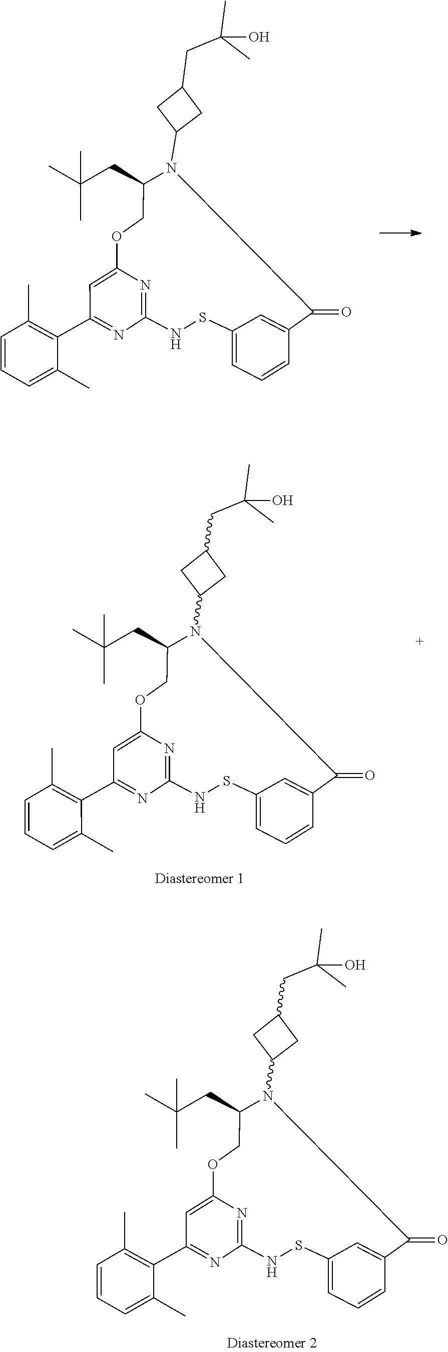 [SMILES: Cc1cccc(C)c1-c1cc2nc(n1)NSc1cccc(c1)C(=O)N(C1CC(CC(C)(C)O)C1)[C@H](CC(C)(C)C)CO2 ; Cc1cccc(C)c1-c1cc2nc(n1)NSc1cccc(c1)C(=O)N(C1CC(CC(C)(C)O)C1)[C@H](CC(C)(C)C)CO2 ; Cc1cccc(C)c1-c1cc2nc(n1)NSc1cccc(c1)C(=O)N(C1CC(CC(C)(C)O)C1)[C@H](CC(C)(C)C)CO2]

(11R)-6-(2,6-dimethylphenyl)-11-(2,2-dimethylpropyl)-12-[3-(2-hydroxy-2-methylpropyl)cyclobutyl]-9-oxa-2λ⁶-thia-3,5,12,19-tetraazatricyclo[12.3.1.14,8]nonadeca-1(17),4(19),5,7,14(18),15-hexaene-2,2,13-trione (22 mg, 0.03544 mmol) (1:1 isomeric mixture) was subjected to SFC separation using a Phenomenex LUX-4 (250×10 mm), 5 uM column at 40° C.; Mobile phase: 32% MeOH (no modifier), 68% CO₂; flow: 10 mL/min; concentration: 24 mg/mL in MeOH (no modifier); injection volume: 70 μL; pressure: 148 bar; wavelength: 210 nm. For each compound, the solvents were evaporated. The residue was transferred into a vial using DCM/hexanes. Evaporation gave the separated isomer as off-a white solid: diastereomer 1, SFC peak 1: (11R)-6-(2,6-dimethylphenyl)-11-(2,2-dimethylpropyl)-12-[3-(2-hydroxy-2-methylpropyl)cyclobutyl]-9-oxa-2λ⁶-thia-3,5,12,19-tetraazatricyclo[12.3.1.14,8]nonadeca-1(17),4(19),5,7,14(18),15-hexaene-2,2,13-trione (5.4 mg, 49%). ESI-MS m/z calc. 620.3032, found 621.28 (M+1)⁺; Retention time: 1.83 minutes (LC method A); and diastereomer 2, SFC peak 2: (11R)-6-(2,6-dimethylphenyl)-11-(2,2-dimethylpropyl)-12-[3-(2-hydroxy-2-methylpropyl)cyclobutyl]-9-oxa-2λ⁶-thia-3,5,12,19-tetraazatricyclo[12.3.1.14,8]nonadeca-1(17),4(19),5,7,14(18),15-hexaene-2,2,13-trione (6.7 mg, 61%). ESI-MS m/z calc. 620.3032, found 621.28 (M+1)⁺; Retention time: 1.82 minutes (LC method A).

### Example 122: Preparation of Compound 154 and Compound 155

### Step 1: 3-[[4-[(2R)-2-[[3-[2-(tert-Butoxycarbonylamino)ethyl]cyclobutyl]amino]-4,4-dimethyl-pentoxy]-6-(2,6-dimethylphenyl)pyrimidin-2-yl]sulfamoyl]benzoic acid

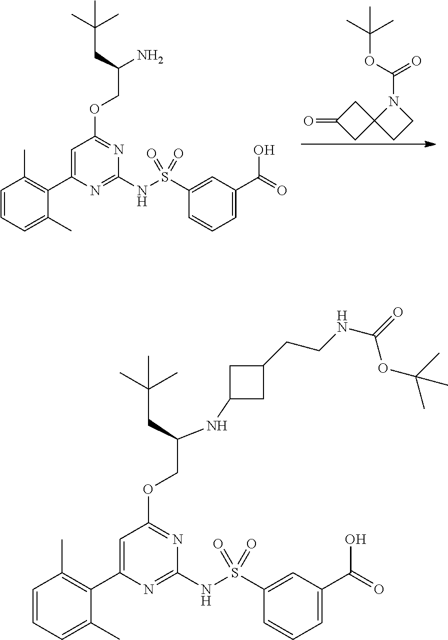 [SMILES: Cc1cccc(C)c1-c1cc(OC[C@H](N)CC(C)(C)C)nc(NS(=O)(=O)c2cccc(C(=O)O)c2)n1 ; CC(C)(C)OC(=O)N1CCC12CC(=O)C2 ; Cc1cccc(C)c1-c1cc(OC[C@@H](CC(C)(C)C)NC2CC(CCNC(=O)OC(C)(C)C)C2)nc(NS(=O)(=O)c2cccc(C(=O)O)c2)n1]

In a reaction vial, 3-[[4-[(2R)-2-amino-4,4-dimethyl-pentoxy]-6-(2,6-dimethylphenyl)pyrimidin-2-yl]sulfamoyl]benzoic acid (hydrochloride salt)(800 mg, 1.457 mmol) was mixed with tert-butyl 6-oxo-1-azaspiro[3.3]heptane-1-carboxylate (308 mg, 1.458 mmol) in methylene chloride (5.2 mL). The reaction mixture was stirred at rt for 15 min then sodium triacetoxyborohydride (618 mg, 2.916 mmol) was added. The reaction was stirred at rt for 1.5 h. then partitioned between ethyl acetate and 1N HCl. The reaction mixture was extracted with ethyl acetate (3×) and the organic layer was washed with sat. NaCl solution. The organic layer was isolated, dried over anhydrous sodium sulfate, filtered, and evaporated to dryness. The crude material was purified by reverse phase chromatography using 20-100% water/ACN gradient. The product fractions were made basic by addition of triethylamine then evaporated to dryness. The residue was partitioned between ethyl acetate and 1N HCl. The aqueous layer was extracted with ethyl acetate. The combined organic layer was washed with sat. NaCl solution, dried over anhydrous sodium sulfate, filtered, and evaporated to dryness to provide two products as white solids: 3-[[4-[(2R)-2-[(1-tert-butoxycarbonyl-1-azaspiro[3.3]heptan-6-yl)amino]-4,4-dimethyl-pentoxy]-6-(2,6-dimethylphenyl)pyrimidin-2-yl]sulfamoyl]benzoic acid (hydrochloride salt)(108 mg, 10%)¹H NMR (400 MHz, DMSO-d₆) δ 9.11 (s, 1H), 8.47 (t, J=1.8 Hz, 1H), 8.14 (t, J=7.8 Hz, 2H), 7.69 (t, J=7.8 Hz, 1H), 7.26 (t, J=7.6 Hz, 1H), 7.13 (d, J=7.6 Hz, 2H), 6.32 (s, 1H), 5.02 (s, 1H), 4.42-4.18 (m, 1H), 4.23-4.03 (m, 1H), 3.87-3.77 (m, 1H), 3.71-3.60 (m, 3H), 2.36-2.18 (m, 5H), 2.19-2.08 (m, 3H), 1.40 (s, 7H), 1.35 (s, 9H), 0.93 (s, 9H). ESI-MS m/z calc. 707.33527, found 708.6 (M+1)⁺; Retention time: 1.93 minutes (LC method A); and 3-[[4-[(2R)-2-[[3-[2-(tert-butoxycarbonylamino)ethyl]cyclobutyl]amino]-4,4-dimethyl-pentoxy]-6-(2,6-dimethylphenyl)pyrimidin-2-yl]sulfamoyl]benzoic acid (hydrochloride salt)(315.4 mg, 28%), ¹H NMR (400 MHz, DMSO-d₆) δ 8.52-8.43 (m, 1H), 8.14 (ddd, J=9.2, 5.2, 1.6 Hz, 2H), 7.70 (t, J=7.8 Hz, 1H), 7.27 (t, J=7.6 Hz, 1H), 7.13 (d, J=7.6 Hz, 2H), 6.78 (q, J=5.3 Hz, 1H), 6.35 (s, 1H), 4.31 (d, J=12.6 Hz, 1H), 4.21-4.07 (m, 1H), 4.03 (q, J=7.1 Hz, 1H), 3.95-3.80 (m, 1H), 3.75-3.55 (m, 1H), 2.96-2.77 (m, 2H), 2.41-2.29 (m, 1H), 2.27-2.17 (m, 1H), 2.17-2.07 (m, 1H), 2.00 (d, J=12.4 Hz, 6H), 1.73-1.60 (m, 1H), 1.63-1.51 (m, 2H), 1.50-1.39 (m, 2H), 1.36 (s, 9H), 0.93 (s, 9H). ESI-MS m/z calc. 709.3509, found 710.5 (M+1)⁺; Retention time: 1.4 minutes (LC method A).

### Step 2: (11R)-12-[3-(2-Aminoethyl)cyclobutyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

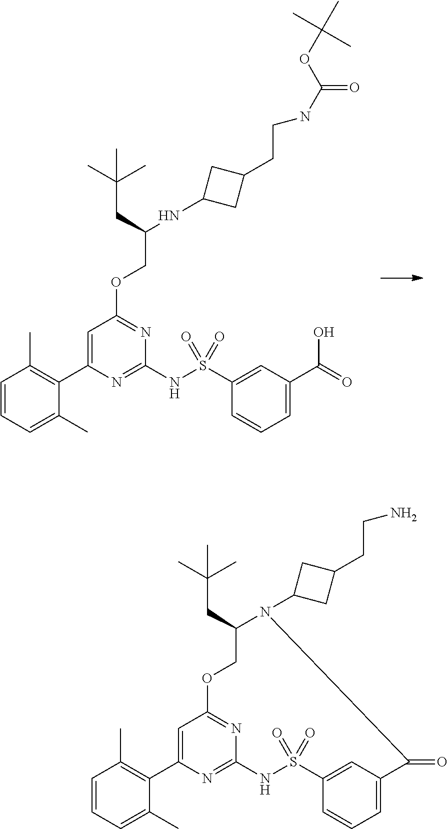 [SMILES: Cc1cccc(C)c1-c1cc(OC[C@@H](CC(C)(C)C)NC2CC(CCNC(=O)OC(C)(C)C)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC(CCN)C1)[C@H](CC(C)(C)C)CO2]

In a reaction vial, 3-[[4-[(2R)-2-[[3-[2-(tert-butoxycarbonylamino)ethyl]cyclobutyl]amino]-4,4-dimethyl-pentoxy]-6-(2,6-dimethylphenyl)pyrimidin-2-yl]sulfamoyl]benzoic acid (hydrochloride salt) (298.6 mg, 0.3841 mmol) was dissolved in dimethylformamide (13.5 mL) along with 4-methylmorpholine (85.3 μL, 0.7759 mmol) and cooled to 0° C. To the reaction, 2-chloro-4,6-dimethoxy-1,3,5-triazine (67.7 mg, 0.3856 mmol) was added and the reaction was allowed to stir at 0° C. for 1.5 h. then an additional 4-methylmorpholine (42.65 μL, 0.3879 mmol) was added. The reaction was allowed to warm to rt and stirred at that temperature overnight. The reaction was concentrated to a third of the volume then partitioned between ethyl acetate and 0.5N HCl solution. The organics were separated, dried over sodium sulfate, and evaporated to dryness. The crude material was purified by column chromatography on silica using 20-80% ethyl acetate/hexanes gradient. The intermediate was isolated as a white solid, tert-butyl N-[2-[3-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]ethyl]carbamate (162.8 mg, 61%), which was subsequently dissolved in methylene chloride (1 mL) along with HCl (118 μL of 4 M, 0.4720 mmol)(4M in dioxane) and stirred at rt for 5 h. The reaction was evaporated to dryness under vacuum to give (11R)-12-[3-(2-aminoethyl)cyclobutyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (131.1 mg, 54%) ESI-MS m/z calc. 591.2879, found 592.3 (M+1)⁺; Retention time: 0.51 minutes (LC method D).

### Step 3: N-(2-{3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(18),4,6,8(19),14,16-hexaen-12-yl]cyclobutyl}ethyl)acetamide, diastereomer 1 (Compound 154), and N-(2-{3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(18),4,6,8(19),14,16-hexaen-12-yl]cyclobutyl}ethyl)acetamide, diastereomer 2 (Compound 155)

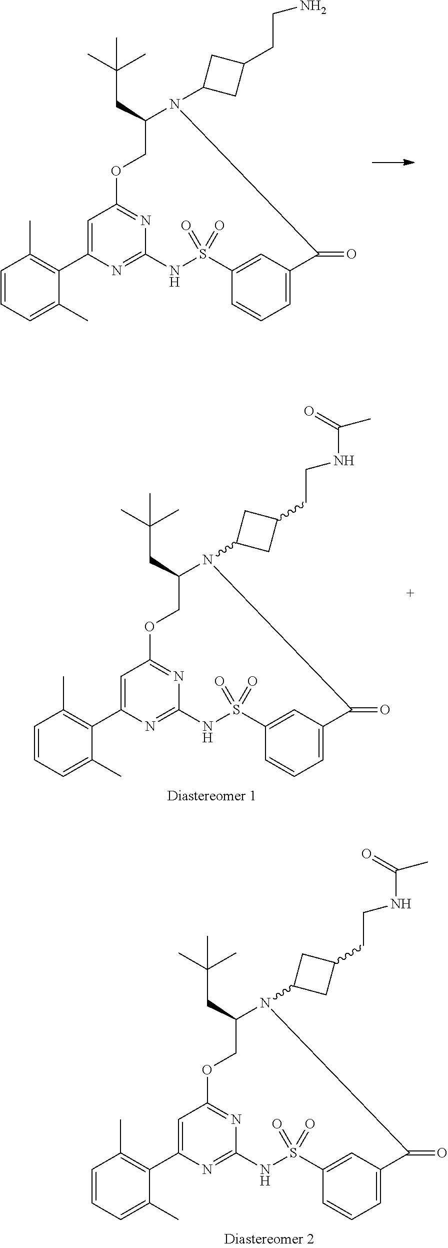 [SMILES: Cc1cccc(C)c1-c1cc2nc(n1)NS(=O)(=O)c1cccc(c1)C(=O)N(C1CC(CCN)C1)[C@H](CC(C)(C)C)CO2 ; CC(=O)NCCC1CC(N2C(=O)c3cccc(c3)S(=O)(=O)Nc3nc(cc(-c4c(C)cccc4C)n3)OC[C@H]2CC(C)(C)C)C1 ; CC(=O)NCCC1CC(N2C(=O)c3cccc(c3)S(=O)(=O)Nc3nc(cc(-c4c(C)cccc4C)n3)OC[C@H]2CC(C)(C)C)C1]

In a reaction vial, (11R)-12-[3-(2-aminoethyl)cyclobutyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (80 mg, 0.1133 mmol) was mixed with diisopropylethylamine (65.2 μL, 0.3743 mmol) in methylene chloride (0.5 mL). To the reaction, acetyl chloride (13.3 μL, 0.1871 mmol) was added and the reaction was stirred at rt for 12 h. The reaction mixture was evaporated to dryness then diluted with ethyl acetate then washed with 1N HCl (3×) and saturated NaCl solution. The organic layer was isolated, dried over anhydrous sodium sulfate, filtered, and evaporated to dryness. The crude material was purified by reverse phase HPLC using 20-80% ACN/water gradient with HCl modifier to give two isomers: Diastereomer 1, N-(2-{3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(18),4,6,8(19),14,16-hexaen-12-yl]cyclobutyl}ethyl)acetamide (49.4 mg, 69%)¹H NMR (400 MHz, Methanol-d₄) δ 8.56 (t, J=1.8 Hz, 1H), 8.02 (dt, J=7.6, 1.5 Hz, 1H), 7.80-7.58 (m, 2H), 7.27 (t, J=7.7 Hz, 1H), 7.14 (d, J=7.6 Hz, 2H), 6.27 (s, 1H), 5.27 (dd, J=10.7, 4.4 Hz, 1H), 4.41-4.05 (m, 2H), 3.83 (dq, J=12.2, 5.0 Hz, 1H), 3.29-3.23 (m, 1H), 3.22-3.10 (m, 3H), 2.58-2.33 (m, 1H), 2.27-1.99 (m, 7H), 1.96 (s, 4H), 1.79 (q, J=7.5 Hz, 2H), 1.65 (dd, J=15.3, 8.0 Hz, 1H), 1.52 (d, J=15.0 Hz, 1H), 0.58 (s, 9H). ESI-MS m/z calc. 633.29846, found 634.2 (M+1)⁺; Retention time: 1.62 minutes (LC method A); and diastereomer 2, N-(2-{3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(18),4,6,8(19),14,16-hexaen-12-yl]cyclobutyl}ethyl)acetamide (23 mg, 32%)¹H NMR (400 MHz, Methanol-d₄) δ 8.56 (d, J=1.8 Hz, 1H), 8.02 (dt, J=7.4, 1.7 Hz, 1H), 7.72 (dd, J=6.9, 2.5 Hz, 1H), 7.71 (s, OH), 7.69 (d, J=7.5 Hz, 1H), 7.27 (t, J=7.6 Hz, 1H), 7.14 (d, J=7.7 Hz, 2H), 6.27 (s, 1H), 5.29 (dd, J=10.7, 4.4 Hz, 1H), 4.25 (t, J=11.2 Hz, 1H), 3.93 (p, J=8.8 Hz, 1H), 3.81 (ddd, J=11.8, 7.8, 4.3 Hz, 1H), 3.17 (t, J=7.1 Hz, 2H), 2.79 (dq, J=34.8, 9.6 Hz, 2H), 2.49-2.30 (m, 2H), 2.18-2.02 (m, J=8.0, 7.4 Hz, 2H), 1.95 (s, 3H), 1.94 (s, 1H), 1.80 (d, J=7.2 Hz, 1H), 1.79-1.67 (m, 2H), 1.51 (d, J=15.1 Hz, 1H), 0.59 (s, 9H). ESI-MS m/z calc. 633.29846, found 634.2 (M+1)⁺; Retention time: 1.66 minutes (LC method A).

### Example 123: Preparation of Compound 156 and Compound 157

### Step 1: 3-Benzyloxy-1-methyl-cyclobutanol

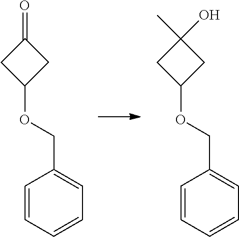

3-benzyloxycyclobutanone (503 mg, 2.8545 mmol) was dissolved in diethylether (1.4 mL) then methyl magnesium bromide 3M in diethylether (1.40 mL of 3 M, 4.2000 mmol) was added drop wise at room temperature. The reaction was stirred for an hour then cooled to 0° C. and quenched with ammonium chloride (5 mL). The mixture was diluted with EtOAc (5 mL) and the layers separated. The aqueous layer was extracted with EtOAc (2×5 mL). The combined organics were dried over sodium sulfate and concentrated. The residue was dry-loaded on to silica gel and purified by flash column chromatography using 0-30% EtOAc in hexanes to give 3-benzyloxy-1-methyl-cyclobutanol (283 mg, 46%) as a colorless oil. ¹H NMR (250 MHz, DMSO-d₆) δ 7.40-7.24 (m, 5H), 4.40-4.29 (m, 2H), 2.33-2.12 (m, 2H), 2.02-1.84 (m, 2H), 1.15 (s, 3H). Diastereomer peak: 1.28 (s, 3H)

### Step 2: [3-Methyl-3-(trifluoromethoxy)cyclobutoxy]methylbenzene

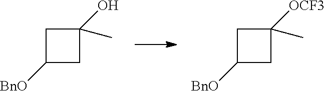 [SMILES: CC1(O)CC(OCc2ccccc2)C1 ; CC1(OC(F)(F)F)CC(OCc2ccccc2)C1]

3-benzyloxy-1-methyl-cyclobutanol (9.23 g, 48.009 mmol) was dissolved in ethyl acetate (325 mL) then silver triflate (37.05 g, 144.20 mmol), Selectfluor (25.61 g, 72.292 mmol) and potassium fluoride (11.02 g, 189.68 mmol) were added. The vessel was flushed with nitrogen and 2-fluoropyridine (14.100 g, 12.5 mL, 145.23 mmol) and trifluoromethyltrimethylsilane (20.683 g, 21.5 mL, 145.46 mmol) were added. The mixture was allowed to stir for 3 days at room temperature under a nitrogen atmosphere. The mixture was filtered through a pad of Celite, and dry-loaded on to silica gel and purified by flash column chromatography using 0-30% ethyl acetate in hexanes. The appropriate fractions were collected to give [3-methyl-3-(trifluoromethoxy)cyclobutoxy]methylbenzene (2.58 g, 19%) as a colorless oil. ¹H NMR (250 MHz, CDCl₃) δ 7.47-7.16 (m, 5H), 4.43 (s, 2H), 3.77 (p, J=6.9 Hz, 1H), 2.49 (d, J=6.3 Hz, 4H), 1.50 (s, 3H). Note: Peak at 5.30 is DCM

### Step 3: 3-Methyl-3-(trifluoromethoxy)cyclobutanol

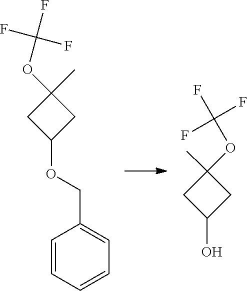 [SMILES: CC1(OC(F)(F)F)CC(OCc2ccccc2)C1 ; CC1(OC(F)(F)F)CC(O)C1]

[3-Methyl-3-(trifluoromethoxy)cyclobutoxy]methylbenzene (635 mg, 2.4399 mmol) was dissolved in methyl acetate (15.875 mL) and Pd/C (683 mg, 10% w/w, 0.6418 mmol) was added. The reaction was placed under a hydrogen atmosphere (balloon) and allowed to stir or 48 h. Celite was added and the solids filtered off and rinsed with diethylether. The filtrate was concentrated to give 3-methyl-3-(trifluoromethoxy)cyclobutanol (364.5 mg, 79%) as a colorless oil. ¹H NMR (250 MHz, CDCl₃) δ 4.04 (p, J=7.0 Hz, 1H), 2.67-2.49 (m, 2H), 2.49-2.32 (m, 2H), 1.87 (bs, 1H), 1.55-1.42 (m, 3H).

### Step 4: (2R)-4,4-Dimethyl-2-[[3-methyl-3-(trifluoromethoxy)cyclobutyl]amino]pentan-1-ol

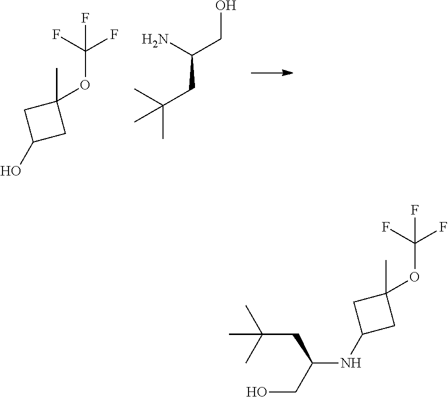

Into a solution of 3-methyl-3-(trifluoromethoxy)cyclobutanol (432 mg, 2.5392 mmol) and pyridine (606.36 mg, 0.62 mL, 7.6657 mmol) in anhydrous DCM (4 mL) was added trifluoromethylsulfonyl trifluoromethanesulfonate (1.0804 g, 0.73 mL, 3.8293 mmol) at 0° C. The reaction was stirred at rt for 2 hours. TLC indicated the disappearance of the starting material. The reaction mixture was diluted with hexane (40 mL) and washed with 1 N HCl (20 mL), saturated sodium bicarbonate (20 mL) and brine (20 mL). The solution was dried over anhydrous sodium sulfate and concentrated under vacuum to furnish the triflate, which was used in the next step reaction without purification. (2R)-2-Amino-4,4-dimethyl-pentan-1-ol (hydrochloride salt) (518 mg, 3.0893 mmol) was suspended in DCM (30 mL), and 2 N sodium carbonate solution (30 mL). Two layers were separated, and the aqueous layer was extracted with DCM (2×30 mL). The organic layers were combined and dried over anhydrous sodium sulfate and concentrated under vacuum. The residue was combined with crude triflate and dissolved in ACN (10 mL). Potassium carbonate (1.77 g, 12.807 mmol) was added to the reaction mixture, which was heated to 60° C. and stirred overnight. The solids were filtered off, before the solvent was removed under vacuum. The residue was purified by silica gel chromatography using 0 to 10% methanol in DCM to furnish (2R)-4,4-dimethyl-2-[[3-methyl-3-(trifluoromethoxy)cyclobutyl]amino]pentan-1-ol (401 mg, 56%) as a yellow oil. ESI-MS m/z calc. 283.1759, found 284.2 (M+1)⁺; Retention time: 3.67 minutes, 3.49 minutes (LC method S), diastereomeric mixture.

### Step 5: 3-[[4-[(2R)-4,4-Dimethyl-2-[[3-methyl-3-(trifluoromethoxy)cyclobutyl]amino]pentoxy]-6-(2,6-dimethylphenyl)pyrimidin-2-yl]sulfamoyl]benzoic acid

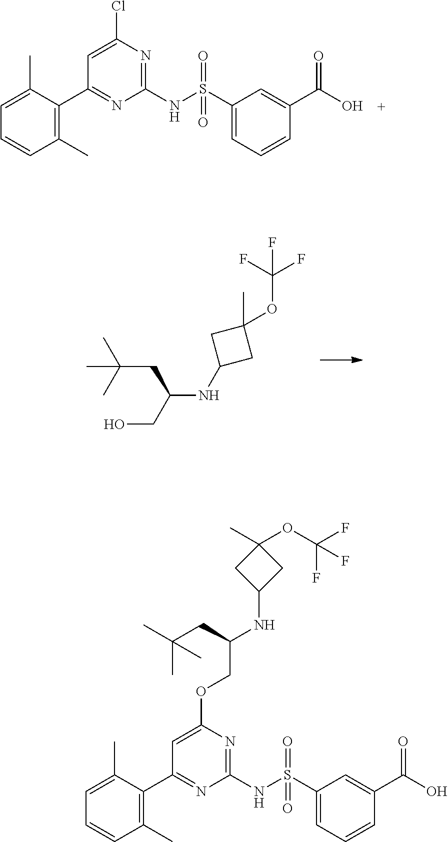 [SMILES: Cc1cccc(C)c1-c1cc(Cl)nc(NS(=O)(=O)c2cccc(C(=O)O)c2)n1 ; CC(C)(C)C[C@H](CO)NC1CC(C)(OC(F)(F)F)C1 ; Cc1cccc(C)c1-c1cc(OC[C@@H](CC(C)(C)C)NC2CC(C)(OC(F)(F)F)C2)nc(NS(=O)(=O)c2cccc(C(=O)O)c2)n1]

Into a solution of (2R)-4,4-dimethyl-2-[[3-methyl-3-(trifluoromethoxy)cyclobutyl]amino]pentan-1-ol (401 mg, 1.415 mmol) and 3-[[4-chloro-6-(2,6-dimethylphenyl)pyrimidin-2-yl]sulfamoyl]benzoic acid (685 mg, 1.6393 mmol) in anhydrous THE (10 mL) was added sodium tert-butoxide (689 mg, 7.1694 mmol) at ambient temperature. The reaction mixture was stirred at rt for 2 hours, then it was quenched with 1 N HCl (aqueous) (25 mL) and diluted with chloroform (25 mL). Two layers were separated, and the aqueous layer was extracted with chloroform (2×25 mL). The combined organic layers were washed with brine (25 mL), dried over anhydrous sodium sulfate and concentrated under vacuum. The residue was purified by silica gel chromatography using 0 to 10% methanol in DCM to furnish 3-[[4-[(2R)-4,4-dimethyl-2-[[3-methyl-3-(trifluoromethoxy)cyclobutyl]amino]pentoxy]-6-(2,6-dimethylphenyl)pyrimidin-2-yl]sulfamoyl]benzoic acid (hydrochloride salt) (897 mg, 72%) as an off-white solid. ESI-MS m/z calc. 664.2542, found 665.2 (M+1)⁺; Retention time: 4.71 minutes, LC method S.

### Step 6: (11R)-6-(2,6-dimethylphenyl)-11-(2,2-dimethylpropyl)-12-[3-methyl-3-(trifluoromethoxy)cyclobutyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound 156), and (11R)-6-(2,6-dimethylphenyl)-11-(2,2-dimethylpropyl)-12-[3-methyl-3-(trifluoromethoxy)cyclobutyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 2 (Compound 157)

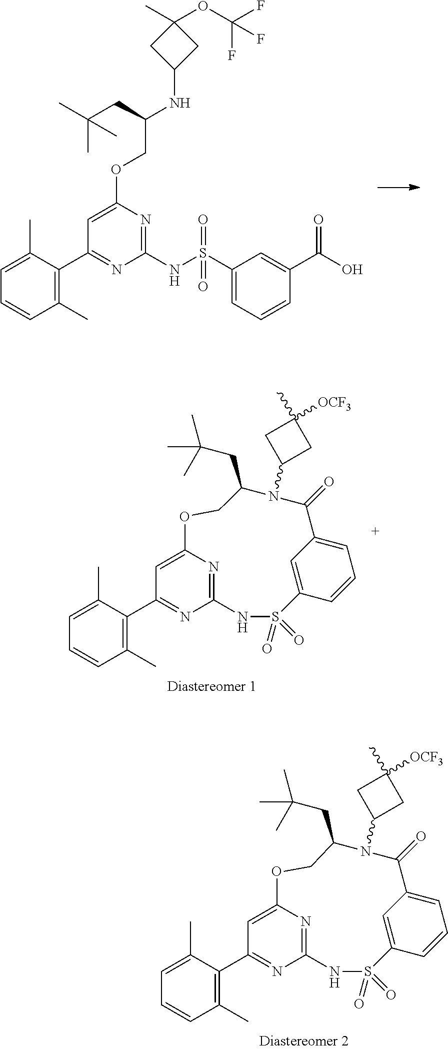

Into a solution of 3-[[4-[(2R)-4,4-dimethyl-2-[[3-methyl-3-(trifluoromethoxy)cyclobutyl]amino]pentoxy]-6-(2,6-dimethylphenyl)pyrimidin-2-yl]sulfamoyl]benzoic acid (897 mg, 1.0795 mmol) and DIEA (1.1130 g, 1.5 mL, 8.6117 mmol) in anhydrous DMF (15 mL) was added a solution of HATU (839 mg, 2.2066 mmol) in anhydrous DMF (15 mL) dropwise. The reaction mixture was stirred at rt overnight. The reaction was quenched with water (20 mL) and extracted with ethyl acetate (3×30 mL). The combined organic layers were washed with brine (3×30 mL), dried over anhydrous sodium sulfate and concentrated under vacuum. The residue was purified by silica gel chromatography using 0 to 30% acetone in hexane to furnish crude mixture of the two diastereomers (˜0.34 g). The crude product was further purified by reverse phase HPLC using 0 to 100% acetonitrile in water (buffered with 0.1% TFA) to furnish: a major isomer, diastereomer 1 (11R)-6-(2,6-dimethylphenyl)-11-(2,2-dimethylpropyl)-12-[3-methyl-3-(trifluoromethoxy)cyclobutyl]-2,2-dioxo-9-oxa-2λ⁶-thia-3,5,12,19-tetrazatricyclo[12.3.1.14,8]nonadeca-1(18),4(19),5,7,14,16-hexaen-13-one (94.4 mg, 13%) as a white powder ¹H NMR (500 MHz, DMSO-d6) δ 8.49 (s, 1H), 7.94 (t, J=3.6 Hz, 1H), 7.70 (d, J=4.8 Hz, 2H), 7.26 (t, J=7.6 Hz, 1H), 7.13 (d, J=7.6 Hz, 2H), 6.42 (s, 1H), 5.11 (dd, J=10.7, 4.5 Hz, 1H), 4.35 (t, J=11.2 Hz, 1H), 4.26 (p, J=8.9 Hz, 1H), 3.87 (m, 1H, overlap with water peak), 3.26 (dd, J=13.8, 8.6 Hz, 1H), 3.18 (dd, J=13.9, 8.5 Hz, 1H), 2.69 (dd, J=13.7, 9.6 Hz, 2H), 2.23-1.79 (m, 6H), 1.73 (s, 3H), 1.54 (dd, J=15.3, 8.3 Hz, 1H), 1.41 (d, J=15.0 Hz, 1H), 0.49 (s, 9H) ESI-MS m/z calc. 646.2437, found 647.6 (M+1)⁺; Retention time: 3.26 minutes, LC method W; and a minor isomer, diastereomer 2 (11R)-6-(2,6-dimethylphenyl)-11-(2,2-dimethylpropyl)-12-[3-methyl-3-(trifluoromethoxy)cyclobutyl]-2,2-dioxo-9-oxa-2λ⁶-thia-3,5,12,19-tetrazatricyclo[12.3.1.14,8]nonadeca-1(18),4(19),5,7,14,16-hexaen-13-one (25.7 mg, 3%) as an off white powder. The minor isomer was purified a second time by SFC using the following condition: normal phase SFC-MS method using a OD-H column (250×21.2 mm, 5 m particle size) sold by Chiral Technologies (pn: 14445), and a dual gradient run from 5-25% mobile phase B over 14.5 minutes. Mobile phase A=CO₂. Mobile phase B=MeOH (20 mM NH3). Flow rate=50-80% MeOH [20 mM NH_{3] 40} mL/min. injection volume=variable, and column temperature=40° C. to give pure diastereomer 2 (11R)-6-(2,6-dimethylphenyl)-11-(2,2-dimethylpropyl)-12-[3-methyl-3-(trifluoromethoxy)cyclobutyl]-2,2-dioxo-9-oxa-2λ⁶-thia-3,5,12,19-tetrazatricyclo[12.3.1.14,8]nonadeca-1(18),4(19),5,7,14,16-hexaen-13-one (14.8 mg, 57%)¹H NMR (400 MHz, DMSO-d₆) δ 12.96 (s, 1H), 8.48 (s, 1H), 7.92 (s, 1H), 7.67 (s, 2H), 7.24 (s, 1H), 7.10 (s, 2H), 6.38 (s, 1H), 5.08 (s, 1H), 4.33 (s, 1H), 3.83 (s, 1H), 3.77 (d, J=16.7 Hz, 1H), 3.64 (s, 1H), 3.53 (s, 1H), 2.37 (s, 2H), 2.02 (s, 6H), 1.60 (d, J=8.7 Hz, 4H), 1.36 (s, 1H), 0.49 (s, 9H). ESI-MS m/z calc. 646.24365, found 647.0 (M+1)⁺; Retention time: 2.18 minutes (LC method A).

### Example 124: Preparation of Compound 158

### Step 1: 3-[[4-[(2R)-2-[[3,3-Bis(isopropoxycarbonyl)cyclobutyl]amino]-4,4-dimethyl-pentoxy]-6-(2,6-dimethylphenyl)pyrimidin-2-yl]sulfamoyl]benzoic acid

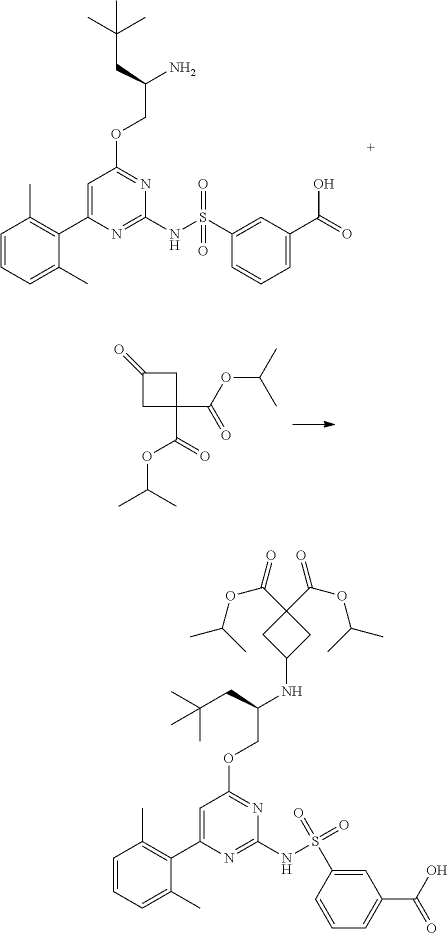 [SMILES: Cc1cccc(C)c1-c1cc(OC[C@H](N)CC(C)(C)C)nc(NS(=O)(=O)c2cccc(C(=O)O)c2)n1 ; CC(C)OC(=O)C1(C(=O)OC(C)C)CC(=O)C1 ; Cc1cccc(C)c1-c1cc(OC[C@@H](CC(C)(C)C)NC2CC(C(=O)OC(C)C)(C(=O)OC(C)C)C2)nc(NS(=O)(=O)c2cccc(C(=O)O)c2)n1]

A 100 mL flask was charged under nitrogen with 3-[[4-[(2R)-2-amino-4,4-dimethyl-pentoxy]-6-(2,6-dimethylphenyl)pyrimidin-2-yl]sulfamoyl]benzoic acid (hydrochloride salt) (1.824 g, 3.322 mmol), anhydrous DCM (5 mL) and DIEA (0.63 mL, 3.617 mmol). The mixture was stirred until almost complete dissolution. acetic acid (0.21 mL, 3.693 mmol) was added followed quickly by diisopropyl 3-oxocyclobutane-1,1-dicarboxylate (0.68 mL, 3.368 mmol). The solution was stirred at room temperature for 20 min. sodium triacetoxyborohydride (1.186 g, 5.596 mmol) was added and the suspension was stirred at room temperature for 3 hours. More diisopropyl 3-oxocyclobutane-1,1-dicarboxylate (0.68 mL, 3.368 mmol) and sodium triacetoxyborohydride (440 mg, 2.076 mmol) were added and the reaction was stirred for an additional 2 hours. The mixture was stored in a freezer at −20° C. overnight. The reaction was let to warm to room temperature, cooled down in ice and quenched by slow addition of aqueous 1N HCl (50 mL). EtOAc (30 mL) and brine (40 mL) were added and the two phases were separated. The aqueous phase was further extracted with EtOAc (2×20 mL). The combined extracts were dried over sodium sulfate and the solvents evaporated. The residue was diluted in diethyl ether (100 mL) and the resulting suspension was stirred for 10 minutes. The product was filtered, washed with ether and dried to give 3-[[4-[(2R)-2-[[3,3-bis(isopropoxycarbonyl)cyclobutyl]amino]-4,4-dimethyl-pentoxy]-6-(2,6-dimethylphenyl)pyrimidin-2-yl]sulfamoyl]benzoic acid (hydrochloride salt) (1.3 g, 50%) as a white solid. ESI-MS m/z calc. 738.32983, found 739.61 (M+1)⁺; Retention time: 1.5 minutes (LC method A).

### Step 2: 1,1-Bis(propan-2-yl) 3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]cyclobutane-1,1-dicarboxylate

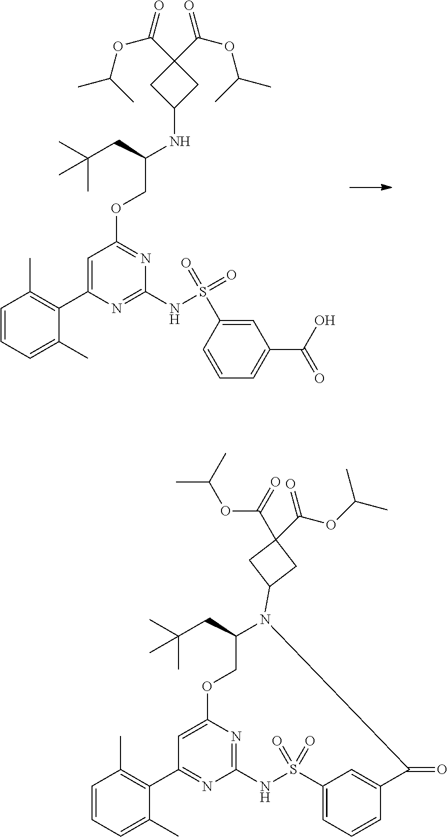 [SMILES: Cc1cccc(C)c1-c1cc(OC[C@@H](CC(C)(C)C)NC2CC(C(=O)OC(C)C)(C(=O)OC(C)C)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC(C(=O)OC(C)C)(C(=O)OC(C)C)C1)[C@H](CC(C)(C)C)CO2]

A 250 mL round bottom flask was charged under nitrogen with COMU (1.60 g, 3.736 mmol), anhydrous DMF (60 mL) and DIEA (1.5 mL, 8.612 mmol). A solution of 3-[[4-[(2R)-2-[[3,3-bis(isopropoxycarbonyl)cyclobutyl]amino]-4,4-dimethyl-pentoxy]-6-(2,6-dimethylphenyl)pyrimidin-2-yl]sulfamoyl]benzoic acid (hydrochloride salt) (1.3 g, 1.677 mmol) (mixture containing approx. 30% of the material) in anhydrous DMF (30 mL) was added dropwise through syringe over a period of 5 minutes. The mixture was stirred at room temperature for 21 h. The reaction was concentrated to a third and diluted with Ethyl acetate (50 mL), 1N aqueous HCl (40 mL) and brine (20 mL). The two phases were separated, and the aqueous phase was further extracted with EtOAc (2×15 mL). The combined extracts were dried over sodium sulfate and the solvents evaporated. The residue was dissolved in DCM and purified by flash chromatography on silica gel (80 g column) using a gradient of ethyl acetate (20 to 100% over 30 min) in hexanes. The product eluted around 50-55% ethyl acetate. After evaporation the residue was triturated in EtOAc/hexanes. Evaporation of the solvents gave 1,1-bis(propan-2-yl) 3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]cyclobutane-1,1-dicarboxylate (244 mg, 20%) as a white solid. ESI-MS m/z calc. 720.3193, found 721.5 (M+1)⁺; Retention time: 2.17 minutes (LC method A).

### Step 3: (11R)-12-[3,3-Bis(hydroxymethyl)cyclobutyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (Compound 158)

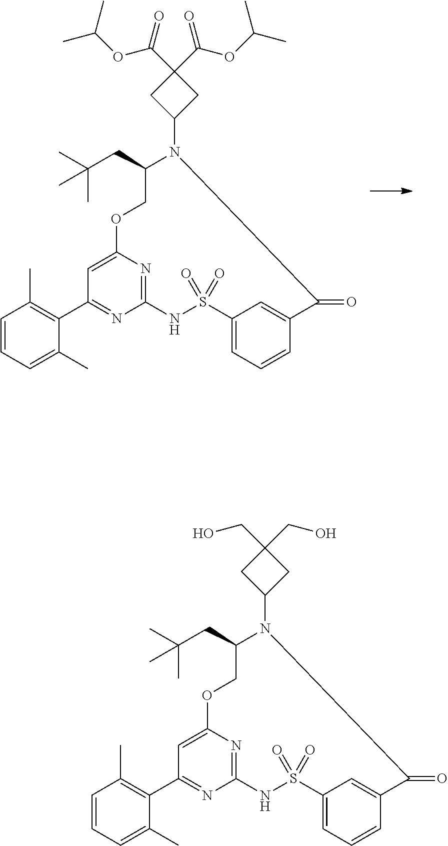 [SMILES: Cc1cccc(C)c1-c1cc2nc(n1)NS(=O)(=O)c1cccc(c1)C(=O)N(C1CC(C(=O)OC(C)C)(C(=O)OC(C)C)C1)[C@H](CC(C)(C)C)CO2 ; Cc1cccc(C)c1-c1cc2nc(n1)NS(=O)(=O)c1cccc(c1)C(=O)N(C1CC(CO)(CO)C1)[C@H](CC(C)(C)C)CO2]

A 4 mL vial was charged with 1,1-bis(propan-2-yl) 3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]cyclobutane-1,1-dicarboxylate (28 mg, 0.03884 mmol), anhydrous THE (0.5 mL). The mixture was cooled in an ice-bath. LiBH4 (0.29 mL of 2 M, 0.5800 mmol) (2M in THF) was added dropwise. The mixture was stirred in an ice bath for 5 min then it was stirred at room temperature for 2.5 h (6% conversion). The reaction was stirred at 70° C. for 30 min (complete conversion). After cooling down, the reaction was quenched by slow addition of acetic acid (100 μL) and methanol (100 L). The solvents were partially evaporated by blowing nitrogen. The remaining solution was diluted with DMSO (1 mL). The solution was microfiltered through a syringe filter disc and purified by reverse phase preparative HPLC (C₁₈) using a gradient of acetonitrile in water (1 to 99% over 15 min) and HCl as a modifier. Evaporation gave (11R)-12-[3,3-bis(hydroxymethyl)cyclobutyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (20.3 mg, 83%) as a white solid. ESI-MS m/z calc. 608.26685, found 609.56 (M+1)⁺; Retention time: 1.45 minutes (LC method A).

### Example 125: Preparation of Compound 159

### Step 1: propan-2-yl 3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]-1-(2-hydroxypropan-2-yl)cyclobutane-1-carboxylate, diastereomer 1 and 2

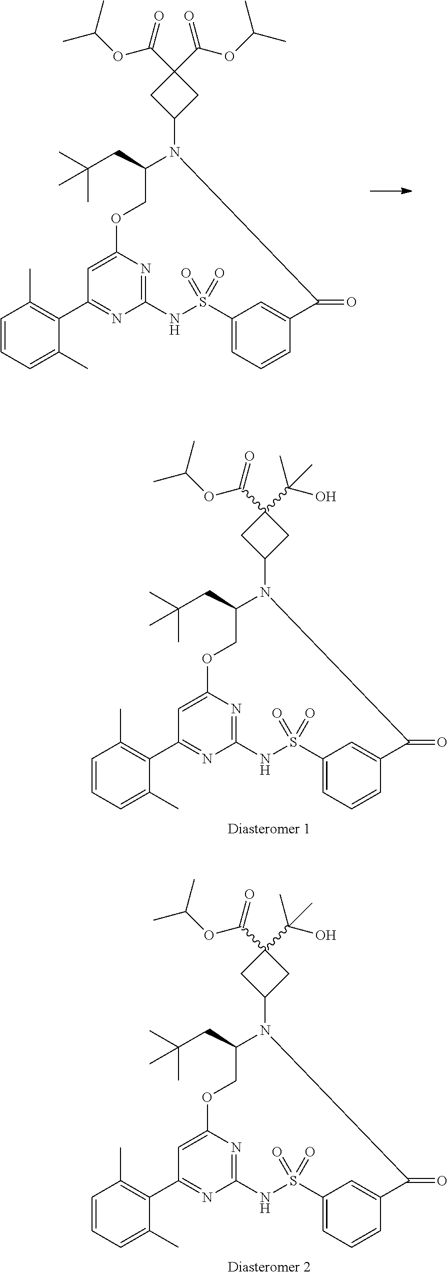

A 4 mL vial was charged under nitrogen with 1,1-bis(propan-2-yl) 3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]cyclobutane-1,1-dicarboxylate (31 mg, 0.04300 mmol), anhydrous THE (0.6 mL) and the solution was cooled down in an ice bath. Bromo(methyl)magnesium (0.08 mL of 3 M, 0.2400 mmol) (3 M solution in diethylether) was added dropwise. The reaction mixture was stirred in the ice bath for 5 min, then it was stirred at room temperature for 2 h. The mixture was cooled down in ice and quenched by adding an aqueous saturated solution of ammonium chloride (2 mL). The product was extracted with EtOAc (3×2 mL). The combined extracts were dried over sodium sulfate and the solvents were evaporated. The residue was dissolved in DMSO (1 mL). The solution was microfiltered through a syringe filter disc and purified by reverse phase preparative HPLC (C₁₈) using a gradient of acetonitrile in water (1 to 99% over 15 min) and HCl as a modifier, which resulted in baseline separation of two isomers. The pure fractions were collected and the solvents evaporated to give both isomers: more polar isomer, diastereomer 1, propan-2-yl 3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]-1-(2-hydroxypropan-2-yl)cyclobutane-1-carboxylate (9 mg, 60%). ESI-MS m/z calc. 692.32434, found 693.29 (M+1)⁺; Retention time: 1.86 minutes (LC method A); and less polar, diastereomer 2. propan-2-yl 3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]-1-(2-hydroxypropan-2-yl)cyclobutane-1-carboxylate (7 mg, 47%). ESI-MS m/z calc. 692.32434, found 693.74 (M+1)⁺; Retention time: 2.03 minutes (LC method A).

### Step 2: (11R)-6-(2,6-Dimethylphenyl)-11-(2,2-dimethylpropyl)-12-[3-(hydroxymethyl)-3-(2-hydroxypropan-2-yl)cyclobutyl]-9-oxa-2λ⁶-thia-3,5,12,19-tetraazatricyclo[12.3.1.14,8]nonadeca-1(17),4(19),5,7,14(18),15-hexaene-2,2,13-trione (Compound 159)

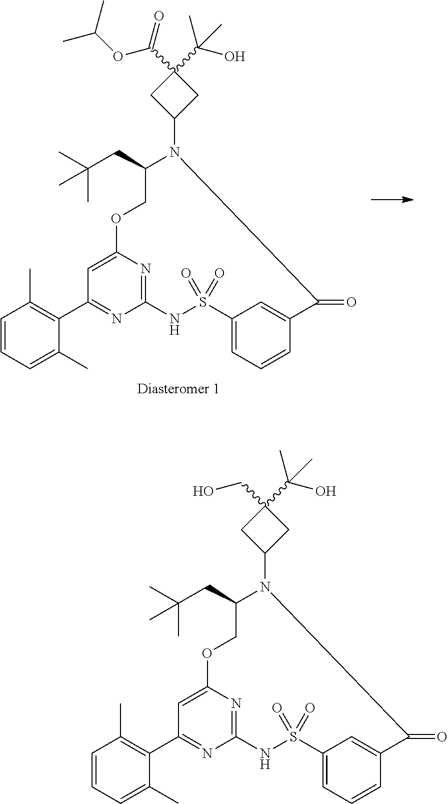

A 4 mL vial was charged under nitrogen with propan-2-yl 3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]-1-(2-hydroxypropan-2-yl)cyclobutane-1-carboxylate (9 mg, 0.01299 mmol) (more polar isomer) and anhydrous THE (0.20 mL). Lithium borohydride (0.09 mL of 2 M, 0.1800 mmol) (2 M in THF) was added and the reaction was stirred at 70° C. for 30 min. After cooling down, the reaction was quenched by slow addition of acetic acid (100 μL) and methanol (100 μL). The solvents were partially evaporated by blowing nitrogen. The remaining solution was diluted with DMSO (1 mL). The solution was microfiltered through a syringe filter disc and purified by reverse phase preparative HPLC (C₁₈) using a gradient of acetonitrile in water (1 to 99% over 15 min) and HCl as a modifier. After evaporation, the product was purified a second time using a shallower gradient 0-50% over 10 min and 50-60% over 20 min of MeCN in water/HCl. Evaporation, trituration in DCM/hexanes and evaporation gave (11R)-6-(2,6-dimethylphenyl)-11-(2,2-dimethylpropyl)-12-[3-(hydroxymethyl)-3-(2-hydroxypropan-2-yl)cyclobutyl]-9-oxa-2λ⁶-thia-3,5,12,19-tetraazatricyclo[12.3.1.14,8]nonadeca-1(17),4(19),5,7,14(18),15-hexaene-2,2,13-trione (4.7 mg, 56%) as a white solid. ESI-MS m/z calc. 636.29816, found 637.58 (M+1)⁺; Retention time: 1.65 minutes (LC method A).

### Example 126: Preparation of Compound 160

### Step 1: (11R)-6-(2,6-Dimethylphenyl)-11-(2,2-dimethylpropyl)-12-[3-(hydroxymethyl)-3-(2-hydroxypropan-2-yl)cyclobutyl]-9-oxa-2λ⁶-thia-3,5,12,19-tetraazatricyclo[12.3.1.14,8]nonadeca-1(17),4(19),5,7,14(18),15-hexaene-2,2,13-trione (Compound 160)

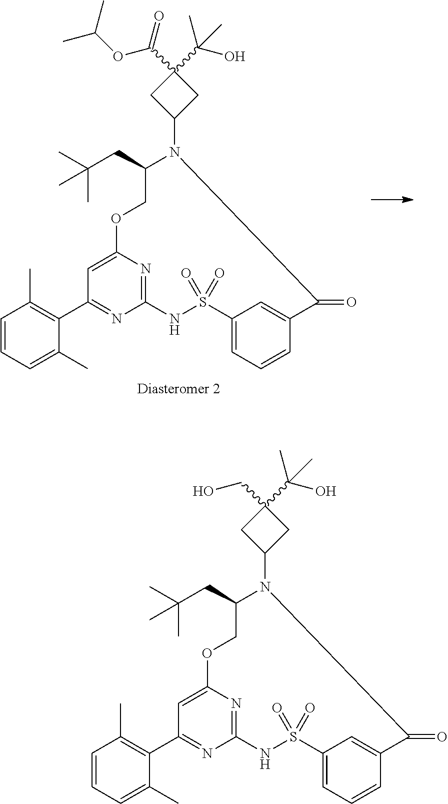

A 4 mL vial was charged under nitrogen with propan-2-yl 3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4(19),5,7,14(18),15-hexaen-12-yl]-1-(2-hydroxypropan-2-yl)cyclobutane-1-carboxylate (7 mg, 0.01010 mmol) (less polar isomer, diastereomer 2), anhydrous THE (0.2 mL). Lithium borohydride (0.09 mL of 2 M, 0.1800 mmol) (2M in THF) was added dropwise and the reaction was stirred at 70° C. for 40 min. After cooling down, the reaction was quenched by slow addition of acetic acid (100 μL) and methanol (100 μL). The solvents were partially evaporated by blowing nitrogen. The remaining solution was diluted with DMSO (1 mL). The solution was microfiltered through a syringe filter disc and purified by reverse phase preparative HPLC (C₁₈) using a gradient of acetonitrile in water (1 to 99% over 15 min) and HCl as a modifier. Evaporation gave (11R)-6-(2,6-dimethylphenyl)-11-(2,2-dimethylpropyl)-12-[3-(hydroxymethyl)-3-(2-hydroxypropan-2-yl)cyclobutyl]-9-oxa-2λ⁶-thia-3,5,12,19-tetraazatricyclo[12.3.1.14,8]nonadeca-1(17),4(19),5,7,14(18),15-hexaene-2,2,13-trione (3.2 mg, 49%) as a white solid. ESI-MS m/z calc. 636.29816, found 637.58 (M+1)⁺; Retention time: 1.61 minutes (LC method A).

### Example 127: Preparation of Compound 161 and Compound 162

### Step 1: Methyl 2-[[(1R)-1-(hydroxymethyl)-3,3-dimethyl-butyl]amino]spiro[3.3]heptane-6-carboxylate

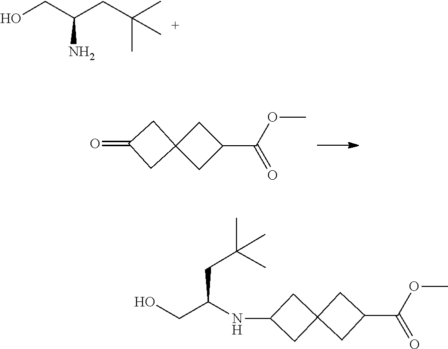

A 500 mL round bottom flask equipped with a magnetic stir bar was charged under nitrogen with methyl 2-oxospiro[3.3]heptane-6-carboxylate (12.18 g, 72.42 mmol) and anhydrous DCE (200 mL). Stirring was commenced and (2R)-2-amino-4,4-dimethyl-pentan-1-ol (hydrochloride salt) (12.2 g, 72.76 mmol) was added. To the suspension, DIEA (15 mL, 86.12 mmol) and acetic acid (4.8 mL, 84.41 mmol) were added and the reaction was stirred at rt for 5-10 min (complete dissolution). sodium triacetoxyborohydride (22.5 g, 106.2 mmol) was added and stirring was continued at rt for 16 h. The reaction was cooled down in an ice-bath (internal temperature 2° C.) and was quenched by slow addition of aqueous HCl (40 mL of 4 M, 160.0 mmol) while maintaining temperature below 7° C. A suspension of sodium bicarbonate (45 g, 535.7 mmol) in water (100 mL) was slowly added (foaming) while maintaining temperature below 10° C. More water (50 mL) and brine (50 mL) were added and the mixture was stirred until gas evolution stopped (final pH=7-8). The two phases were separated, and the aqueous phase was further extracted with DCM (3×50 mL). The combined extracts were washed with brine (50 mL), dried over sodium sulfate and filtered through a pad of Celite. Evaporation of the solvents gave methyl 2-[[(1R)-1-(hydroxymethyl)-3,3-dimethyl-butyl]amino]spiro[3.3]heptane-6-carboxylate (21.08 g, 100%) as a white solid. ESI-MS m/z calc. 283.21475, found 284.12 (M+1)⁺; Retention time: 0.88 minutes; LC method A. ¹H NMR (400 MHz, DMSO-d₆) δ 7.77 (broad s, 1H), 5.23 (broad s, 1H), 3.57 (s, 3H), 3.54-3.42 (m, 2H), 3.31 (dd, J=11.8, 5.9 Hz, 1H), 3.09-2.97 (m, 1H), 2.73 (br s, 1H), 2.40-1.95 (m, 8H), 1.39 (dd, J=14.4, 7.6 Hz, 1H), 1.25 (dd, J=14.4, 2.4 Hz, 1H), 0.89 (s, 9H).

### Step 2: (2R)-2-[[6-(1-Hydroxy-1-methyl-ethyl)spiro[3.3]heptan-2-yl]amino]-4,4-dimethyl-pentan-1-ol

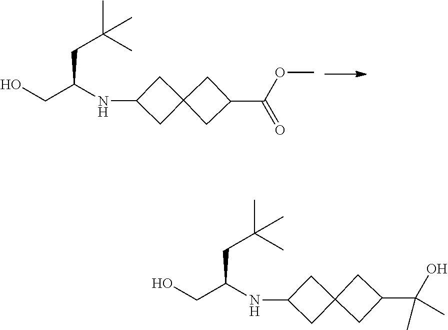 [SMILES: COC(=O)C1CC2(CC(N[C@@H](CO)CC(C)(C)C)C2)C1 ; CC(C)(C)C[C@H](CO)NC1CC2(C1)CC(C(C)(C)O)C2]

A 100 mL flask was charged under nitrogen with methyl 2-[[(1R)-1-(hydroxymethyl)-3,3-dimethyl-butyl]amino]spiro[3.3]heptane-6-carboxylate (322 mg, 1.136 mmol) and anhydrous THE (7 mL). The mixture was cooled down in an ice bath and MeMgBr (1.7 mL of 3 M, 5.100 mmol) (3M in diethyl ether) was added dropwise through syringe over a period of 5 minutes (gas evolution visible at the beginning of the addition). At the end of the addition (clear solution), the ice bath was removed, and the reaction was stirred at room temperature for 1 hour. More THE (4 mL) was added to ease stirring. After 30 min, more MeMgBr (0.2 mL of 3 M, 0.6000 mmol) was added and the mixture was stirred at room temperature for 6 h. The reaction was cooled down and treated with saturated ammonium chloride (50 mL), Brine (30 mL) and EtOAc (40 mL). The two phases were separated, and the aqueous phase was extracted with EtOAc (3×30 mL). The combined extracts were washed with brine (40 mL) dried over sodium sulfate and the solvents evaporated to give crude (2R)-2-[[6-(1-hydroxy-1-methyl-ethyl)spiro[3.3]heptan-2-yl]amino]-4,4-dimethyl-pentan-1-ol (288 mg, 89%) as a colorless resin. ESI-MS m/z calc. 283.25113, found 284.18 (M+1)⁺; Retention time: 0.91 minutes; LC method A.

### Step 3: 6-[[4-(2,6-Dimethylphenyl)-6-[(2R)-2-[[6-(1-hydroxy-1-methyl-ethyl)spiro[3.3]heptan-2-yl]amino]-4,4-dimethyl-pentoxy]pyrimidin-2-yl]sulfamoyl]pyridine-2-carboxylic acid

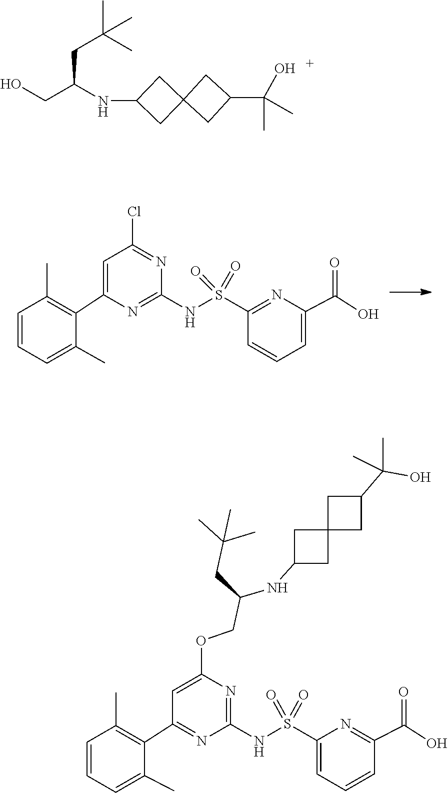 [SMILES: CC(C)(C)C[C@H](CO)NC1CC2(C1)CC(C(C)(C)O)C2 ; Cc1cccc(C)c1-c1cc(Cl)nc(NS(=O)(=O)c2cccc(C(=O)O)n2)n1 ; Cc1cccc(C)c1-c1cc(OC[C@@H](CC(C)(C)C)NC2CC3(C2)CC(C(C)(C)O)C3)nc(NS(=O)(=O)c2cccc(C(=O)O)n2)n1]

In a 100 mL round bottom flask equipped with a magnetic stir bar and a nitrogen line was charged with 6-[[4-chloro-6-(2,6-dimethylphenyl)pyrimidin-2-yl]sulfamoyl]pyridine-2-carboxylic acid (717 mg, 1.712 mmol), (2R)-2-[[6-(1-hydroxy-1-methyl-ethyl)spiro[3.3]heptan-2-yl]amino]-4,4-dimethyl-pentan-1-ol (501 mg, 1.768 mmol), and anhydrous THE (5 mL) (suspension). sodium tert-butoxide (890 mg, 9.261 mmol) was added (slight exotherm observed). More THE (4 mL) was added and the mixture was stirred at room temperature for 1.5 h. More sodium tert-butoxide (489 mg, 5.088 mmol) was added and the mixture was stirred for an additional 30 min. The reaction was then partitioned between ethyl acetate (50 mL), a 1M HCl solution (50 mL) and brine (50 mL). The aqueous phase was further extracted with EtOAc (2×30 mL). The combined organics were washed with brine (50 mL) dried over sodium sulfate, filtered over a pad of Celite and evaporated to dryness. The residue was triturated in EtOAc/diethyl ether (1:3 mixture, approximatively 150 mL). The suspension was stirred at room temperature overnight. The solid was filtered and dried to give crude 6-[[4-(2,6-dimethylphenyl)-6-[(2R)-2-[[6-(1-hydroxy-1-methyl-ethyl)spiro[3.3]heptan-2-yl]amino]-4,4-dimethyl-pentoxy]pyrimidin-2-yl]sulfamoyl]pyridine-2-carboxylic acid (hydrochloride salt) (657 mg, 53%) as an off-white solid. The crude solid was used for the next step without any further purification. ESI-MS m/z calc. 665.3247, found 666.37 (M+1)⁺; Retention time: 1.32 minutes; LC method A.

### Step 4: (11R)-6-(2,6-dimethylphenyl)-11-(2,2-dimethylpropyl)-12-[6-(2-hydroxypropan-2-yl)spiro[3.3]heptan-2-yl]-9-oxa-2λ⁶-thia-3,5,12,18,19-pentaazatricyclo[12.3.1.14,8]nonadeca-1(17),4(19),5,7,14(18),15-hexaene-2,2,13-trione, diastereomer 1 (Compound 161), and (11R)-6-(2,6-dimethylphenyl)-11-(2,2-dimethylpropyl)-12-[6-(2-hydroxypropan-2-yl)spiro[3.3]heptan-2-yl]-9-oxa-2%⁶-thia-3,5,12,18,19-pentaazatricyclo[12.3.1.14,8]nonadeca-1(17),4(19),5,7,14(18),15-hexaene-2,2,13-trione, diastereomer 2 (Compound 162)

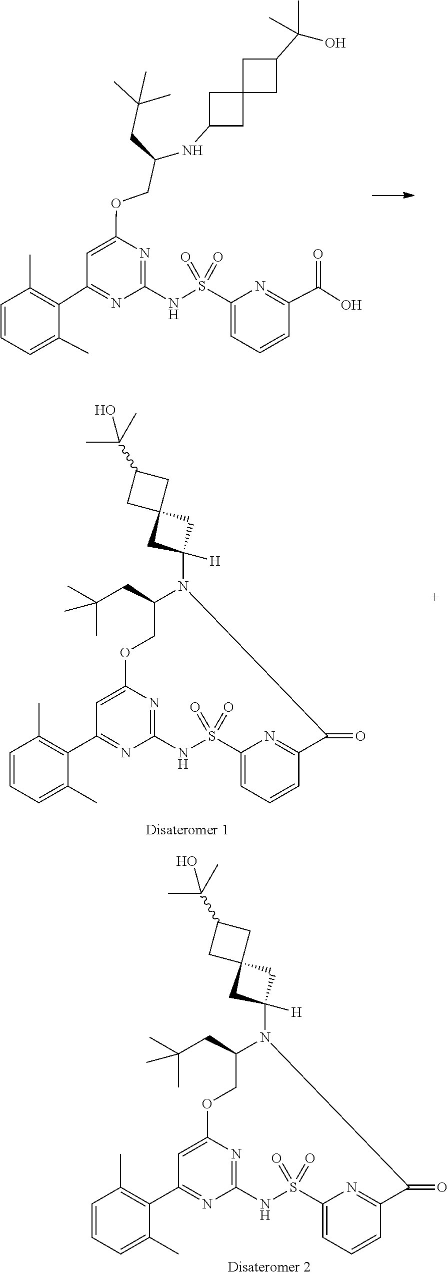

A 100 m round bottom flask was charged under nitrogen with PGP [dimethylamino(triazolo[4,5-b]pyridin-3-yloxy)methylene]-dimethyl-ammonium (Phosphorus Hexafluoride Ion) (747 mg, 1.965 mmol) (HATU), anhydrous DMF (15 mL) and DIEA (0.83 mL, 4.765 mmol). A solution of crude 6-[[4-(2,6-dimethylphenyl)-6-[(2R)-2-[[6-(1-hydroxy-1-methyl-ethyl)spiro[3.3]heptan-2-yl]amino]-4,4-dimethyl-pentoxy]pyrimidin-2-yl]sulfamoyl]pyridine-2-carboxylic acid (hydrochloride salt) (657 mg, 0.9355 mmol) in anhydrous DMF (20 mL) was added dropwise over a period of 5 minutes. The mixture was stirred at room temperature for 17 h. The solvent was evaporated, and the residue was dissolved in DCM. The product was purified by flash chromatography on silica gel (40 g column) using a gradient of ethyl acetate (0 to 100% over 30 min) in hexanes. The product eluted around 70-85% EA. Evaporation of the solvents gave a crude solid that was dissolved in DMSO (2 mL). The solution was microfiltered through a syringe filter disc and purified by reverse phase preparative HPLC (C₁₈) using a gradient of acetonitrile in water (10 to 60% over 30 min) and HCl as a modifier. Two isomers were baseline separated. For each compound, the organic solvent was evaporated. The resulting precipitate was extracted with DCM and the organic phase was dried over sodium sulfate. Evaporation, trituration in DCM/hexanes and evaporation gave the two isomers as a white solid: Diastereomer 1, more polar isomer, (11R)-6-(2,6-dimethylphenyl)-11-(2,2-dimethylpropyl)-12-[6-(2-hydroxypropan-2-yl)spiro[3.3]heptan-2-yl]-9-oxa-2λ⁶-thia-3,5,12,18,19-pentaazatricyclo[12.3.1.14,8]nonadeca-1(17),4(19),5,7,14(18),15-hexaene-2,2,13-trione (86 mg, 28%). ESI-MS m/z calc. 647.31415, found 648.31 (M+1)⁺; Retention time: 1.83 minutes (LC method A), ¹H NMR (400 MHz, DMSO-d₆) δ 13.50-11.75 (broad m, 1H), 8.17 (s, 1H), 8.05 (s, 1H), 7.75 (s, 1H), 7.27 (t, J=7.6 Hz, 1H), 7.13 (s, 2H), 6.38 (s, 1H), 5.66-5.47 (m, 1H), 4.12 (t, J=10.5 Hz, 1H), 3.98 (s, 1H), 3.90 (p, J=8.9 Hz, 1H), 3.60-3.44 (m, 1H), 3.07 (t, J=9.5 Hz, 1H), 2.98 (t, J=9.8 Hz, 1H), 2.31-2.24 (m, 1H), 2.21-1.86 (m, 11H), 1.81 (td, J=8.0, 4.1 Hz, 1H), 1.56 (dd, J=15.1, 8.2 Hz, 1H), 1.44-1.35 (m, 1H), 0.97 (s, 3H), 0.97 (s, 3H), 0.49 (s, 9H); and diastereomer 2, less polar isomer, (11R)-6-(2,6-dimethylphenyl)-11-(2,2-dimethylpropyl)-12-[6-(2-hydroxypropan-2-yl)spiro[3.3]heptan-2-yl]-9-oxa-2λ⁶-thia-3,5,12,18,19-pentaazatricyclo[12.3.1.14,8]nonadeca-1(17),4(19),5,7,14(18),15-hexaene-2,2,13-trione (90 mg, 29%), ESI-MS m/z calc. 647.31415, found 648.31 (M+1)⁺; Retention time: 1.88 minutes (LC method A); ¹H NMR (400 MHz, DMSO-d₆) δ 13.34-11.83 (m, 1H), 8.17 (s, 1H), 8.06 (s, 1H), 7.76 (s, 1H), 7.27 (t, J=7.6 Hz, 1H), 7.13 (s, 2H), 6.38 (s, 1H), 5.68-5.52 (m, 1H), 4.09 (t, J=10.6 Hz, 1H), 4.00 (s, 1H), 3.90 (p, J=8.8 Hz, 1H), 3.58-3.46 (m, 1H), 3.07 (t, J=9.6 Hz, 1H), 2.98 (t, J=9.8 Hz, 1H), 2.38-2.28 (m, 1H), 2.24-1.86 (m, 11H), 1.81 (td, J=7.8, 4.0 Hz, 1H), 1.59 (dd, J=15.1, 8.3 Hz, 1H), 1.44-1.35 (m, 1H), 0.97 (s, 6H), 0.50 (s, 9H).

### Example 128: Preparation of Compound 163

### Step 1: (2R)-2-(Spiro[2.3]hexan-5-ylamino)-3-[1-(trifluoromethyl)cyclopropyl]propan-1-ol

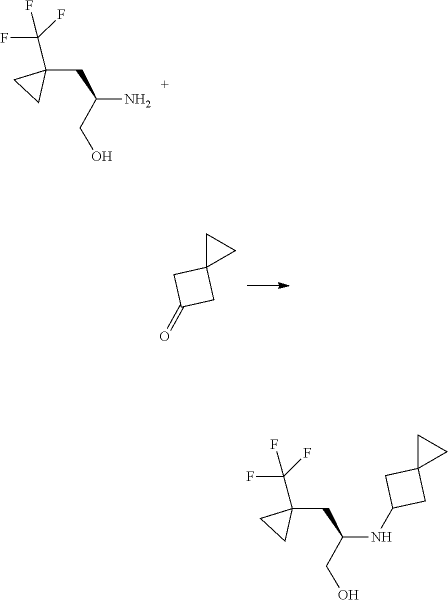 [SMILES: N[C@@H](CO)CC1(C(F)(F)F)CC1 ; O=C1CC2(CC2)C1 ; OC[C@@H](CC1(C(F)(F)F)CC1)NC1CC2(CC2)C1]

To a stirred milky emulsion of (2R)-2-amino-3-[1-(trifluoromethyl)cyclopropyl]propan-1-ol (hydrochloride salt) (600 mg, 2.732 mmol) in anhydrous 1,2-dichloroethane (15 mL) was added a solution of spiro[2.3]hexan-5-one (330 mg, 3.433 mmol) in anhydrous 1,2-dichloroethane (3 mL), followed by addition of glacial acetic acid (200 μL, 3.517 mmol), at ambient temperature under nitrogen. The mixture was stirred for 30 min, then solid sodium triacetoxyborohydride (2.22 g, 10.47 mmol) was added in 3 batches (equal amounts) in 2 min intervals. The reaction was stirred overnight (13 h). The suspension was cooled in an ice-water bath and quenched by slow addition of aqueous hydrochloric acid (6 mL of 2.0 M, 12.00 mmol) to pH about 1.0. The emulsion (no clear phases) was stirred for 20 min. The resulting cooled (ice-bath) suspension was basified by slow addition of sodium carbonate (1.50 g, 14.15 mmol) (Caution! strong effervescence) to adjust pH about 10. The heterogeneous phases were separated, and the aqueous layer was extracted with methylene chloride (2×40 mL). The combined organics were washed with brine (20 mL), dried over sodium sulfate, filtered, and evaporated under reduced pressure to give (2R)-2-(spiro[2.3]hexan-5-ylamino)-3-[1-(trifluoromethyl)cyclopropyl]propan-1-ol (698 mg, 97%) as clear thick oil, which became solid overnight. ESI-MS m/z calc. 263.1497, found 264.1 (M+1)⁺; Retention time: 0.91 minutes; LC method A, ¹H NMR (500 MHz, DMSO-d₆) δ 3.48 (p, J=7.4 Hz, 1H), 3.39 (dd, J=10.6, 4.6 Hz, 1H), 3.28 (dd, J=10.8, 5.2 Hz, 1H), 2.66-2.58 (m, 1H), 2.24-2.04 (m, 2H), 2.00-1.81 (m, 2H), 1.78-1.61 (m, 1H), 1.52 (dd, J=14.9, 7.1 Hz, 1H), 1.00-0.72 (m, 4H), 0.41 (dd, J=9.0, 6.2 Hz, 2H), 0.33 (dd, J=8.8, 5.9 Hz, 2H).

### Step 2: 6-[[4-(2,6-Dimethylphenyl)-6-[(2R)-2-(spiro[2.3]hexan-5-ylamino)-3-[1-(trifluoromethyl)cyclopropyl]propoxy]pyrimidin-2-yl]sulfamoyl]pyridine-2-carboxylic acid

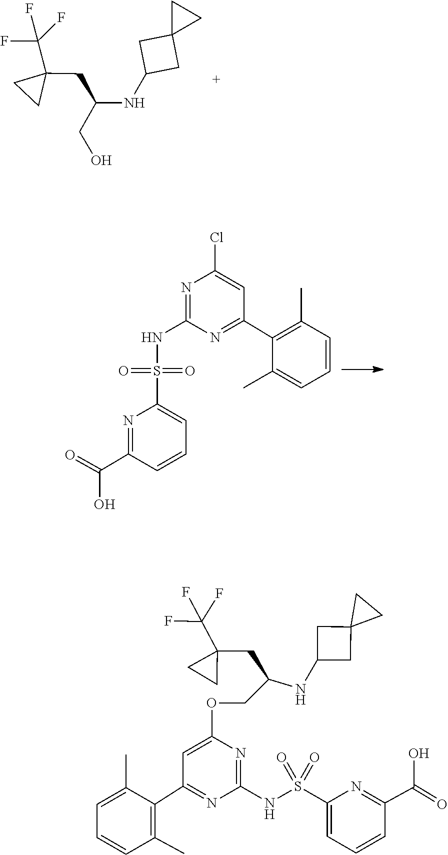 [SMILES: OC[C@@H](CC1(C(F)(F)F)CC1)NC1CC2(CC2)C1 ; Cc1cccc(C)c1-c1cc(Cl)nc(NS(=O)(=O)c2cccc(C(=O)O)n2)n1 ; Cc1cccc(C)c1-c1cc(OC[C@@H](CC2(C(F)(F)F)CC2)NC2CC3(CC3)C2)nc(NS(=O)(=O)c2cccc(C(=O)O)n2)n1]

To a solution of (2R)-2-(spiro[2.3]hexan-5-ylamino)-3-[1-(trifluoromethyl)cyclopropyl]propan-1-ol (4.0 g, 15.19 mmol) and 6-[[4-chloro-6-(2,6-dimethylphenyl)pyrimidin-2-yl]sulfamoyl]pyridine-2-carboxylic acid (5.26 g, 12.56 mmol) in 2-MeTHF (60 mL) was added sodium tert-butoxide (4.4 g, 45.78 mmol) portion-wise keeping the reaction temperature <40° C. The addition was exothermic and the reaction temperature was controlled using rate of base addition. The reaction was stirred for 90 min at room temperature ˜15% SM remained. Added additional sodium tert-butoxide (1.1 g, 11.45 mmol) and stirred for an additional 90 min (warmed a few times). The reaction was quenched with the slow addition of HCl (14 mL of 6 M, 84.00 mmol) and stirred for 5 min. The mixture was transferred to a separatory funnel using 2Me-THF and water. The aqueous phase was separated and extracted with 100 mL of 2Me-THF. The combined organic phases were washed with 100 mL of brine, dried over magnesium sulfate, filtered and concentrated in vacuo. The dark yellow foam was used without further purification. 6-[[4-(2,6-Dimethylphenyl)-6-[(2R)-2-(spiro[2.3]hexan-5-ylamino)-3-[1-(trifluoromethyl)cyclopropyl]propoxy]pyrimidin-2-yl]sulfamoyl]pyridine-2-carboxylic acid (hydrochloride salt) (7.49 g, 87%). ESI-MS m/z calc. 645.22327, found 646.3 (M+1)⁺; Retention time: 1.24 minutes; LC method A.

### Step 3: (11R)-6-(2,6-Dimethylphenyl)-2,2-dioxo-12-spiro[2.3]hexan-5-yl-11-[[1-(trifluoromethyl)cyclopropyl]methyl]-9-oxa-2λ⁶-thia-3,5,12,18,19-pentazatricyclo[12.3.1.14,8]nonadeca-1(18),4(19),5,7,14,16-hexaen-13-one (Compound 163)

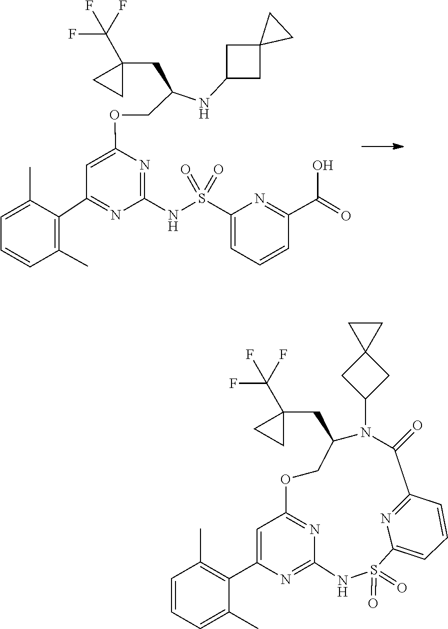 [SMILES: Cc1cccc(C)c1-c1cc(OC[C@@H](CC2(C(F)(F)F)CC2)NC2CC3(CC3)C2)nc(NS(=O)(=O)c2cccc(C(=O)O)n2)n1 ; Cc1cccc(C)c1-c1cc2nc(n1)NS(=O)(=O)c1cccc(n1)C(=O)N(C1CC3(CC3)C1)[C@H](CC1(C(F)(F)F)CC1)CO2]

To a solution of 6-[[4-(2,6-dimethylphenyl)-6-[(2R)-2-(spiro[2.3]hexan-5-ylamino)-3-[1-(trifluoromethyl)cyclopropyl]propoxy]pyrimidin-2-yl]sulfamoyl]pyridine-2-carboxylic acid (hydrochloride salt) (7.49 g, 10.98 mmol) in NMP (110 mL) was added DIEA (6.1 mL, 35.02 mmol) followed by HATU (6.2 g, 16.31 mmol). The mixture was stirred at ambient temperature for 16 h. To the reaction mixture was slowly added HCl (275 mL of 0.2 M, 55.00 mmol) and stirred at ambient temperature for 30 min. The yellow slurry was filtered using a M frit. The solid was diluted with 400 mL of EtOAc (gave a milky emulsion). Added additional 100 mL of EtOAc and 100 mL of DCM. Added additional 200 mL of MeTHF to give a clear solution. The aqueous phase was separated, and the organic phase was washed with 300 mL of brine. The organic phase was separated, dried over magnesium sulfate, filtered and concentrated in vacuo affording a dark yellow foam. The foam was diluted with 150 mL of ACN and stirred at ambient temperature for 1 h. The solid was collected using an M frit and washed with ACN. The filtrate was concentrated in vacuo. The residue was diluted with ACN and heated affording a clear solution which was seeded with product. The resulting precipitate was collected using an M frit and washed with cold MeCN to give (11R)-6-(2,6-Dimethylphenyl)-2,2-dioxo-12-spiro[2.3]hexan-5-yl-11-[[1-(trifluoromethyl)cyclopropyl]methyl]-9-oxa-2λ⁶-thia-3,5,12,18,19-pentazatricyclo[12.3.1.14,8]nonadeca-1(18),4(19),5,7,14,16-hexaen-13-one (4.0 g, 58%). ESI-MS m/z calc. 627.2127, found 628.3 (M+1)⁺; Retention time: 2.59 minutes; LCMS LC method I. This material was combined with another batch before purification.

Combined batches of (11R)-6-(2,6-dimethylphenyl)-2,2-dioxo-12-spiro[2.3]hexan-5-yl-11-[[1-(trifluoromethyl)cyclopropyl]methyl]-9-oxa-2λ⁶-thia-3,5,12,18,19-pentazatricyclo[12.3.1.14,8]nonadeca-1(18),4(19),5,7,14,16-hexaen-13-one (5.2 g, 8.285 mmol) were dissolved in DMSO (25 mL) and chromatographed on an 450 g Reverse phase column eluting with 40-100% ACN/Water. The fractions containing pure product were combined and concentrated in vacuo removing most of the ACN. The precipitated product was collected using an M frit and the solid washed with water. The solid was air dried for 1 h, then in vacuo for 1 h at 45° C. The solid was dried for 16 h in a vacuum oven at 45° C. with a slow nitrogen bleed affording an off-white free flowing solid. (11R)-6-(2,6-dimethylphenyl)-2,2-dioxo-12-spiro[2.3]hexan-5-yl-11-[[1-(trifluoromethyl)cyclopropyl]methyl]-9-oxa-2λ⁶-thia-3,5,12,18,19-pentazatricyclo[12.3.1.14,8]nonadeca-1(18),4(19),5,7,14,16-hexaen-13-one (4.38 g, 84%). ESI-MS m/z calc. 627.2127, found 628.3 (M+1)⁺; Retention time: 1.84 minutes; LC method A, ¹H NMR (500 MHz, DMSO-d₆) δ 13.15 (s, 1H), 8.20 (s, 1H), 8.07 (s, 1H), 7.85 (s, 1H), 7.31 (s, 1H), 7.17 (s, 2H), 6.43 (s, 1H), 5.47 (d, J=7.1 Hz, 1H), 4.25 (q, J=11.8, 8.7 Hz, 2H), 4.00 (s, 1H), 3.43 (t, J=9.4 Hz, 1H), 2.29-2.05 (m, 6H), 1.96 (s, 3H), 1.60 (dd, J=16.5, 9.6 Hz, 1H), 0.87 (d, J=5.2 Hz, 1H), 0.79 (d, J=9.2 Hz, 1H), 0.71 (s, 1H), 0.53 (dt, J=17.1, 7.6 Hz, 6H).

### Example 129: Preparation of Compound 164

### Step 1: 6-[[4-(2,6-Dimethylphenyl)-6-[(2R)-4-methyl-2-(spiro[2.3]hexan-5-ylamino)pentoxy]pyrimidin-2-yl]sulfamoyl]pyridine-2-carboxylic acid

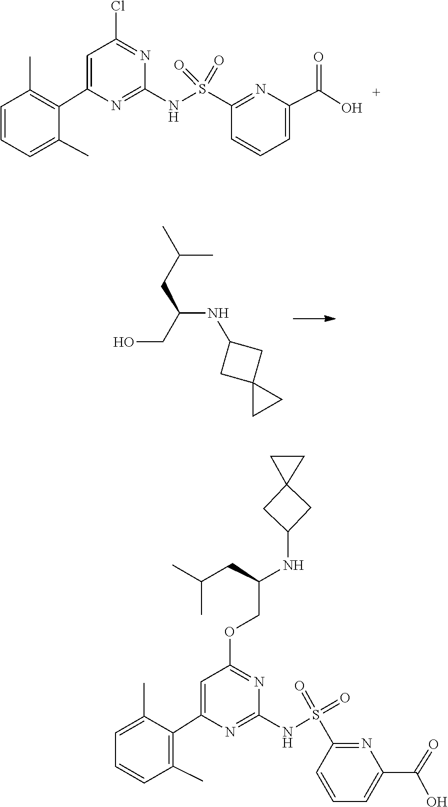

Nitrogen was bubbled through a stirred mixture of 6-[[4-chloro-6-(2,6-dimethylphenyl)pyrimidin-2-yl]sulfamoyl]pyridine-2-carboxylic acid (170 mg, 0.4059 mmol) and (2R)-4-methyl-2-(spiro[2.3]hexan-5-ylamino)pentan-1-ol (hydrochloride salt) (105 mg, 0.4491 mmol) in anhydrous tetrahydrofuran (12 mL). To the resulting emulsion, was added potassium tert-butoxide (185 mg, 1.649 mmol) at once. The reaction was stirred for 4 h at room temperature. The reaction mixture was partitioned between ethyl acetate (50 mL) and hydrochloric acid (2.0 mL of 1 M, 2.000 mmol) (pH 1-2). The aqueous layer was extracted with ethyl acetate (2×30 mL). The combined organics were washed with brine (20 mL), dried over anhydrous sodium sulfate, filtered and concentrated under reduced pressure. The crude was purified by reverse-phase HPLC (Cis column) using 1-99% acetonitrile in water (5 mM HCl as modifier) over 15 min to give 6-[[4-(2,6-dimethylphenyl)-6-[(2R)-4-methyl-2-(spiro[2.3]hexan-5-ylamino)pentoxy]pyrimidin-2-yl]sulfamoyl]pyridine-2-carboxylic acid (hydrochloride salt) (71 mg, 28%), obtained as a white solid. ESI-MS m/z calc. 579.2515, found 580.3 (M+1)⁺; Retention time: 1.26 minutes; LC method A. ¹H NMR (500 MHz, DMSO-d₆) δ 8.25 (q, J=4.5 Hz, 1H), 8.21 (d, J=4.8 Hz, 2H), 7.27 (t, J=7.5 Hz, 1H), 7.14 (d, J=7.7 Hz, 2H), 6.35 (s, 1H), 4.30-4.22 (m, 1H), 4.18-4.12 (m, 1H), 4.02 (q, J=7.8 Hz, 1H), 3.46-3.39 (m, 2H), 2.56 (dt, J=13.3, 6.9 Hz, 2H), 2.24-2.18 (m, 1H), 2.17-2.11 (m, 1H), 2.03 (s, 6H), 1.66-1.58 (m, 1H), 1.54 (dt, J=8.4, 5.3 Hz, 2H), 0.92 (d, J=6.7 Hz, 3H), 0.91 (d, J=6.7 Hz, 3H), 0.54-0.48 (m, 2H), 0.47-0.40 (m, 2H).

### Step 2: (11R)-6-(2,6-Dimethylphenyl)-11-isobutyl-2,2-dioxo-12-spiro[2.3]hexan-5-yl-9-oxa-2λ⁶-thia-3,5,12,18,19-pentazatricyclo[12.3.1.14,8]nonadeca-1(18),4(19),5,7,14,16-hexaen-13-one (Compound 164)

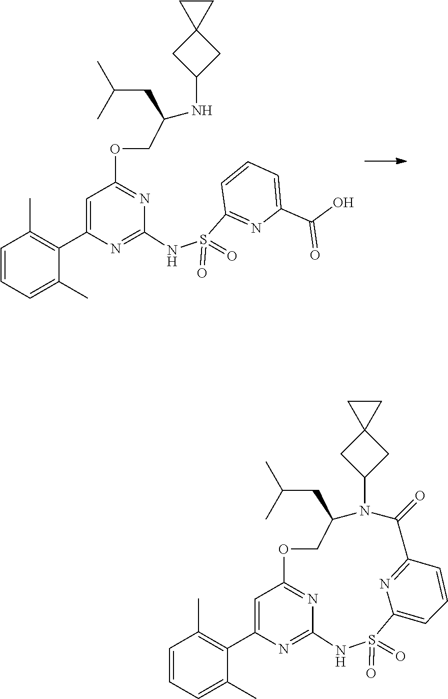 [SMILES: Cc1cccc(C)c1-c1cc(OC[C@@H](CC(C)C)NC2CC3(CC3)C2)nc(NS(=O)(=O)c2cccc(C(=O)O)n2)n1 ; Cc1cccc(C)c1-c1cc2nc(n1)NS(=O)(=O)c1cccc(n1)C(=O)N(C1CC3(CC3)C1)[C@H](CC(C)C)CO2]

In a 20 mL vial, to a stirred solution of 6-[[4-(2,6-dimethylphenyl)-6-[(2R)-4-methyl-2-(spiro[2.3]hexan-5-ylamino)pentoxy]pyrimidin-2-yl]sulfamoyl]pyridine-2-carboxylic acid (hydrochloride salt) (65 mg, 0.1055 mmol) in anhydrous DMF (3.5 mL) were added [dimethylamino(triazolo[4,5-b]pyridin-3-yloxy)methylene]-dimethyl-ammonium (Phosphorus Hexafluoride Ion) (52 mg, 0.1368 mmol) (HATU) and DIEA (75 μL, 0.4306 mmol), in that order. Nitrogen gas was purged for 20 sec and the vial was capped. The reaction was stirred at ambient temperature for 15 min. The reaction was microfiltered and purified from reverse-phase HPLC (Cis column, using 1-99% acetonitrile in water, 5 mM HCl as modifier, over 15 min) to give (11R)-6-(2,6-dimethylphenyl)-11-isobutyl-2,2-dioxo-12-spiro[2.3]hexan-5-yl-9-oxa-2λ⁶-thia-3,5,12,18,19-pentazatricyclo[12.3.1.14,8]nonadeca-1(18),4(19),5,7,14,16-hexaen-13-one (41 mg, 68%), obtained as white solid. ESI-MS m/z calc. 561.24097, found 562.4 (M+1)⁺; Retention time: 1.93 minutes; LC method A. ¹H NMR (500 MHz, DMSO-d₆) δ 12.74 (s, 1H), 8.19 (t, J=7.8 Hz, 1H), 8.06 (d, J=7.9 Hz, 1H), 7.81 (d, J=7.7 Hz, 1H), 7.27 (t, J=7.7 Hz, 1H), 7.14 (d, J=7.6 Hz, 2H), 6.34 (s, 1H), 5.61 (dd, J=9.7, 4.9 Hz, 1H), 4.29 (p, J=8.5 Hz, 1H), 4.19 (t, J=10.6 Hz, 1H), 3.62 (tt, J=11.0, 4.2 Hz, 1H), 3.36 (q, J=8.9 Hz, 2H), 2.19-2.10 (m, 2H), 2.04 (s, 6H), 1.67 (ddd, J=14.1, 10.5, 3.3 Hz, 1H), 1.36 (dtt, J=12.7, 9.3, 4.7 Hz, 1H), 1.23 (ddd, J=13.6, 10.0, 3.1 Hz, 1H), 0.74 (d, J=6.6 Hz, 3H), 0.57-0.51 (m, 2H), 0.49 (tt, J=9.1, 4.1 Hz, 2H), 0.29 (d, J=6.3 Hz, 3H).

### Example 130: Preparation of Compound 165

### Step 1: 3-[[4-[(2R)-2-[[3-(tert-Butoxycarbonylamino)cyclobutyl]amino]-4,4-dimethyl-pentoxy]-6-(2,6-dimethylphenyl)pyrimidin-2-yl]sulfamoyl]benzoic acid

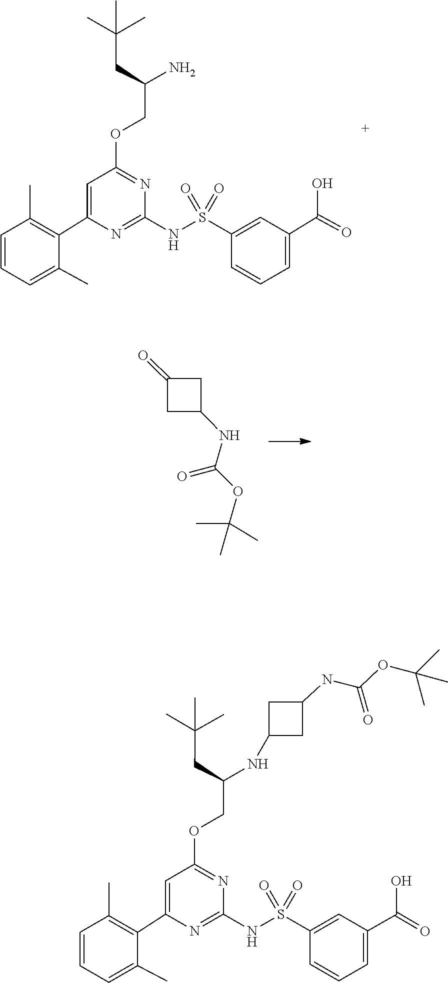

3-[[4-[(2R)-2-Amino-4,4-dimethyl-pentoxy]-6-(2,6-dimethylphenyl)pyrimidin-2-yl]sulfamoyl]benzoic acid (hydrochloride salt) (2.968 g, 5.405 mmol), tert-butyl N-(3-oxocyclobutyl)carbamate (1.208 g, 6.522 mmol), and sodium triacetoxyborohydride (3.88 g, 18.31 mmol) were combined in DCM (9 mL) and stirred at room temperature for 6 h. There was still starting material remaining. More sodium triacetoxyborohydride (1.49 g, 7.030 mmol) was added and the reaction was stirred for 1 h. Still no change so more tert-butyl N-(3-oxocyclobutyl)carbamate (0.5 g, 2.699 mmol) was added and the reaction was stirred an additional 4 h. The reaction was quenched with methanol, then partitioned between ethyl acetate and 1M HCl solution. The organics were washed twice more with 1M HCl, then brine. The organics were dried over sodium sulfate and evaporated The solid was triturated with ether and further dried to give 3-[[4-[(2R)-2-[[3-(tert-butoxycarbonylamino)cyclobutyl]amino]-4,4-dimethyl-pentoxy]-6-(2,6-dimethylphenyl)pyrimidin-2-yl]sulfamoyl]benzoic acid (hydrochloride salt) (3.36 g, 52%). Reaction was taken on crude to the next step. ESI-MS m z calc. 681.31964, found 682.2 (M+1)⁺; Retention time: 0.55 minutes; LC method D.

### Step 2: tert-Butyl N-[3-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]carbamate, diastereomer 1, and tert-butyl N-[3-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]carbamate, diastereomer 2

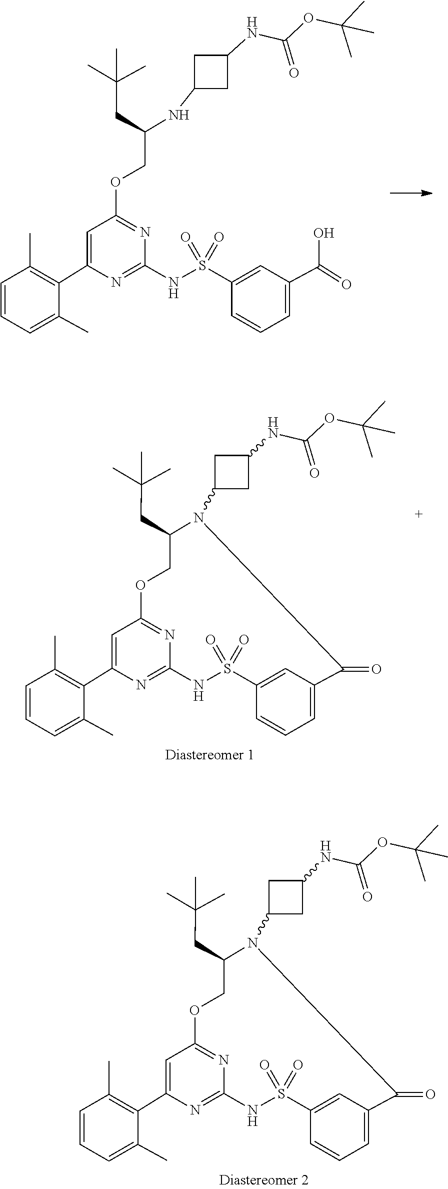 [SMILES: Cc1cccc(C)c1-c1cc(OC[C@@H](CC(C)(C)C)NC2CC(NC(=O)OC(C)(C)C)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC(NC(=O)OC(C)(C)C)C1)[C@H](CC(C)(C)C)CO2 ; Cc1cccc(C)c1-c1cc2nc(n1)NS(=O)(=O)c1cccc(c1)C(=O)N(C1CC(NC(=O)OC(C)(C)C)C1)[C@H](CC(C)(C)C)CO2]

3-[[4-[(2R)-2-[[3-(tert-butoxycarbonylamino)cyclobutyl]amino]-4,4-dimethyl-pentoxy]-6-(2,6-dimethylphenyl)pyrimidin-2-yl]sulfamoyl]benzoic acid (hydrochloride salt) (3.283 g, 2.742 mmol) was dissolved in DMF (10 mL) and added to a stirred solution of 4-(6-cyano-2-methyl-7-oxo-4,8-dioxa-2,5-diazadec-5-en-3-ylidene)morpholin-4-ium hexafluorophosphate(V) (1.21 g, 2.825 mmol) and triethylamine (1.6 mL, 11.48 mmol) in DMF (25 mL). The reaction mixture was stirred at room temperature for 16 h, then evaporated. The crude material was purified by silica gel chromatography eluting with 30-100% ethyl acetate in hexanes to give a mixture of the cis/trans cyclized products. The cis/trans products were further purified by reverse phase HPLC (200 mg/injection) on a 50 mm×100 mm column eluting with 40-80% ACN in 5 mmol aqueous HCl. The desired fractions were evaporated to give the two products. Diastereomer 1, Peak 1: tert-butyl N-[3-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]carbamate (612 mg, 34%). ESI-MS m/z calc. 663.3091, found 664.3 (M+1)⁺; Retention time: 1.94 minutes; LC method A; and diastereomer 2, peak 2: tert-butyl N-[3-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]carbamate (334 mg, 18%), ESI-MS m/z calc. 663.3091, found 664.3 (M+1)⁺; Retention time: 1.99 minutes; LC method A.

### Step 3: (11R)-12-(3-Aminocyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 165)

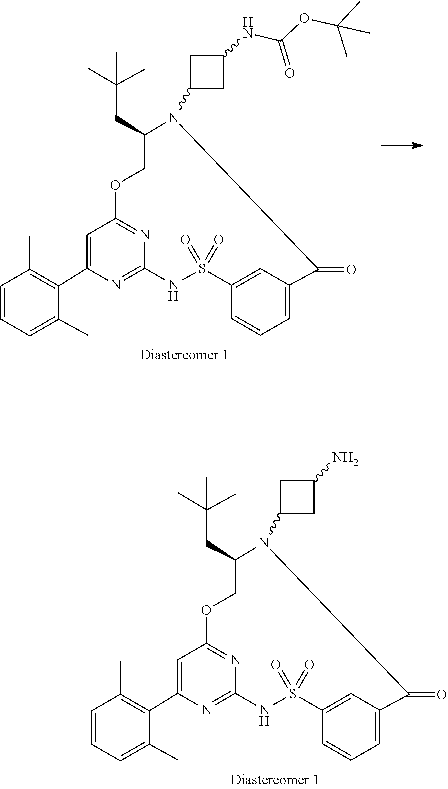 [SMILES: Cc1cccc(C)c1-c1cc2nc(n1)NS(=O)(=O)c1cccc(c1)C(=O)N(C1CC(NC(=O)OC(C)(C)C)C1)[C@H](CC(C)(C)C)CO2 ; Cc1cccc(C)c1-c1cc2nc(n1)NS(=O)(=O)c1cccc(c1)C(=O)N(C1CC(N)C1)[C@H](CC(C)(C)C)CO2]

tert-butyl N-[3-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]carbamate diastereomer 1, (58.4 mg, 0.08797 mmol) was dissolved in 4M HCl in dioxane (1.5 mL of 4 M, 6.000 mmol) and stirred at room temperature for 30 min. The reaction was evaporated to dryness and used without further purification to give diastereomer 1 (11R)-12-(3-aminocyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (52 mg, 98%). ESI-MS m/z calc. 563.25665, found 564.7 (M+1)⁺; Retention time: 0.49 minutes; LC method D.

A small amount of (11R)-12-(3-aminocyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (15 mg, 0.02124 mmol) was purified by LC/MS utilizing a gradient of 1-99% acetonitrile in 5 mM aqueous HCl to yield (11R)-12-(3-aminocyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (11.2 mg, 88%). ESI-MS m/z calc. 563.25665, found 564.7 (M+1)⁺; Retention time: 1.14 minutes; LC method A. ¹H NMR (400 MHz, DMSO-d₆) δ 13.13 (s, 1H), 8.49 (s, 1H), 8.18 (d, J=5.1 Hz, 3H), 7.95 (s, 1H), 7.69 (s, 2H), 7.26 (t, J=7.7 Hz, 1H), 7.17-7.05 (m, 2H), 6.42 (s, 1H), 5.14 (dd, J=10.8, 4.4 Hz, 1H), 4.48 (p, J=8.3 Hz, 1H), 4.13 (t, J=11.3 Hz, 1H), 3.96-3.84 (m, 1H), 3.77-3.68 (m, 1H), 3.33-3.15 (m, 2H), 2.38 (s, 2H), 2.01 (d, J=78.0 Hz, 6H), 1.54-1.35 (m, 2H), 0.49 (s, 9H).

### Example 131: Preparation of Compound 166

### Step 1: (11R)-12-(3-Aminocyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 166)

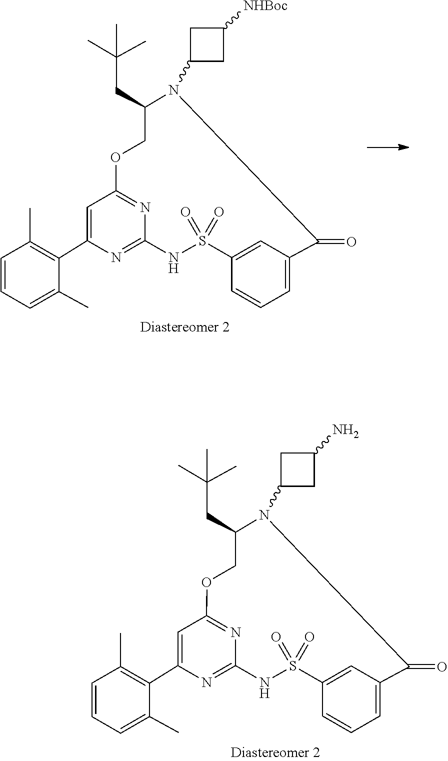 [SMILES: Cc1cccc(C)c1-c1cc2nc(n1)NS(=O)(=O)c1cccc(c1)C(=O)N(C1CC(NC(=O)OC(C)(C)C)C1)[C@H](CC(C)(C)C)CO2 ; Cc1cccc(C)c1-c1cc2nc(n1)NS(=O)(=O)c1cccc(c1)C(=O)N(C1CC(N)C1)[C@H](CC(C)(C)C)CO2]

(11R )-12-(3-aminocyclobutyl-6-2,6-dimethylphenyl-11-2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) diastereomer 2 (13 mg, 0.01906 mmol), prepared by a manner analogous to that described above for diastereomer 1, was purified by LC/MS utilizing a gradient of 1-99% acetonitrile in 5 mM aqueous HCl to yield (11R)-12-(3-aminocyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt)(7.2 mg, 63%) ESI-MS m/z calc. 563.25665, found 564.7 (M+1)⁺; Retention time: 1.2 minutes. (LC method A).

### Example 132: Preparation of Compound 167

### Step 1: (11R)-6-(2,6-dimethylphenyl)-11-(2,2-dimethylpropyl)-12-[3-(2-oxo-1,3-oxazolidin-3-yl)cyclobutyl]-9-oxa-2λ⁶-thia-3,5,12,19-tetraazatricyclo[12.3.1.14,8]nonadeca-1(18),4,6,8(19),14,16-hexaene-2,2,13-trione (Compound 167)

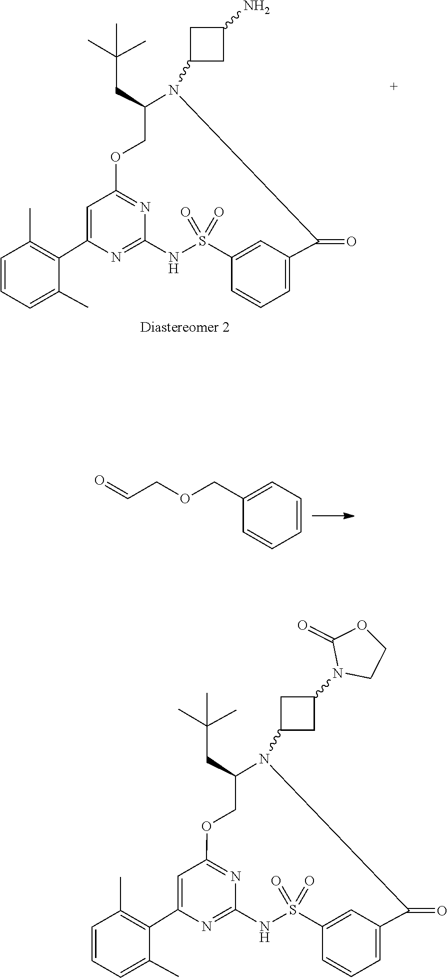 [SMILES: Cc1cccc(C)c1-c1cc2nc(n1)NS(=O)(=O)c1cccc(c1)C(=O)N(C1CC(N)C1)[C@H](CC(C)(C)C)CO2 ; O=CCOCc1ccccc1 ; Cc1cccc(C)c1-c1cc2nc(n1)NS(=O)(=O)c1cccc(c1)C(=O)N(C1CC(N3CCOC3=O)C1)[C@H](CC(C)(C)C)CO2]

(11R)-12-(3-aminocyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (diastereomer 2, 40 mg, 0.05865 mmol) was combined with 2-benzoyloxyacetaldehyde (9 mg, 0.05993 mmol) in DCM (0.5 mL) and stirred for 20 minutes. The reaction mixture was cooled to 0° C. in an ice bath, then sodium triacetoxyborohydride (37 mg, 0.1746 mmol) was added, and the reaction mixture was stirred for one hour. A second portion of 2-benzoyloxyacetaldehyde (4 mg, 0.02664 mmol) was added leading to nearly complete conversion with a small amount of dialkylation. The reaction mixture was partitioned between 1M HCl and ethyl acetate. The layers were separated and the aqueous was extracted an additional three times with ethyl acetate. The combined organics were washed with brine, dried over sodium sulfate and concentrated to give as a white solid (˜15% double alkylation, but used without purification in the next step (11R)-12-(3-{[2-(benzyloxy)ethyl]amino}cyclobutyl)-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione (hydrochloride salt) (47 mg, 109%) ESI-MS m/z calc. 697.3298, found 698.5 (M+1)⁺; Retention time: 0.62 minutes; LC method D.

The product was combined with methyl chloroformate (11 mg, 0.1164 mmol) in DCM (0.5 mL) and DIPEA (50 μL, 0.2871 mmol) was added. The reaction was stirred for 10 minutes at room temperature, then was partitioned between 1M HCl and ethyl acetate. The aqueous layer was extracted 3× with ethyl acetate, and the combined organics were washed with brine and dried over sodium sulfate. The resulting material (still carrying the impurity from step 1) was used in the next step without further purification. methyl N-[2-(benzyloxy)ethyl]-N-{3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(18),4,6,8(19),14,16-hexaen-12-yl]cyclobutyl}carbamate (42 mg, 95%). ESI-MS m/z calc. 755.33527, found 756.6 (M+1)⁺; Retention time: 0.81 minutes; LC method D.

The product was combined with dihydroxypalladium (15 mg, 0.01068 mmol) in methanol (1.5 mL) in a nitrogen-purged vial. hydrogen gas was bubbled in through a balloon for 10 minutes, then the reaction was stirred at room temperature for an additional 3 hours with the hydrogen balloon in place. The reaction vial was then purged with nitrogen, and the reaction mixture was diluted with methanol and filtered through Celite to give as a white solid after drying, methyl N-{3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(18),4,6,8(19),14,16-hexaen-12-yl]cyclobutyl}-N-(2-hydroxyethyl)carbamate (25 mg, 64%), which was used in the next step without purification (with the now doubly-debenzylated impurity still present). ESI-MS m z calc. 665.2883, found 666.4 (M+1)⁺; Retention time: 0.65 minutes; LC method D.

The product was dissolved in THE (1.5 mL) and sodium tert-butoxide (28 mg, 0.2914 mmol) was added in one portion. After 5 minutes the reaction mixture was partitioned between 1M HCl and ethyl acetate. The layers were separated, and the aqueous was extracted an additional three times with ethyl acetate, and the combined organics were washed with brine, dried over sodium sulfate, and concentrated. The resulting crude material was dissolved in 1:1 DMSO/methanol, filtered, and purified by reverse phase HPLC (1-99% ACN in water, HCl modifier, 15 min run) to give as the major product (11R)-6-(2,6-dimethylphenyl)-11-(2,2-dimethylpropyl)-12-[3-(2-oxo-1,3-oxazolidin-3-yl)cyclobutyl]-9-oxa-2λ⁶-thia-3,5,12,19-tetraazatricyclo[12.3.1.14,8]nonadeca-1(18),4,6,8(19),14,16-hexaene-2,2,13-trione (14.3 mg, 38%) ESI-MS m/z calc. 633.2621, found 634.6 (M+1)⁺; Retention time: 1.62 minutes (LC method A).

### Example 133: Preparation of Compound 168

### Step 1: Methyl N-{3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(18),4,6,8(19),14,16-hexaen-12-yl]cyclobutyl}carbamate (Compound 168)

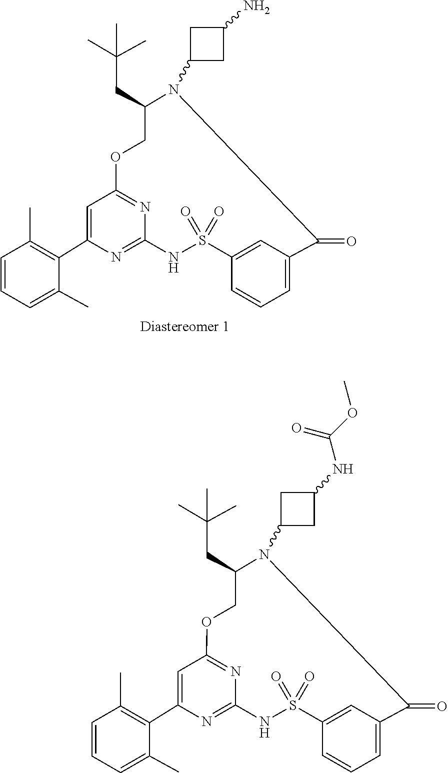 [SMILES: Cc1cccc(C)c1-c1cc2nc(n1)NS(=O)(=O)c1cccc(c1)C(=O)N(C1CC(N)C1)[C@H](CC(C)(C)C)CO2 ; COC(=O)NC1CC(N2C(=O)c3cccc(c3)S(=O)(=O)Nc3nc(cc(-c4c(C)cccc4C)n3)OC[C@H]2CC(C)(C)C)C1]

(11R)-12-(3-aminocyclobutyl)-6-(2,6-dimethylphenyl)-11-(2,2-dimethylpropyl)-2,2-dioxo-9-oxa-2 λ6-thia-3,5,12,19-tetrazatricyclo[12.3.1.14,8]nonadeca-1(18),4(19),5,7,14,16-hexaen-13-one, diastereomer 1 (hydrochloride salt) (30 mg, 0.04249 mmol), methyl chloroformate (8 μL, 0.1035 mmol), and DIEA (37 μL, 0.2124 mmol) were combined in DCM (0.5 mL) and stirred at room temperature for 15 min. The reaction mixture was evaporated and the crude material was purified by reverse-phase HPLC utilizing a gradient of 10-99% acetonitrile in 5 mM aqueous HCl to yield methyl N-{3-[(11R)-6-(2,6-dimethylphenyl)-11-(2,2-dimethylpropyl)-2,2,13-trioxo-9-oxa-2 λ6-thia-3,5,12,19-tetraazatricyclo[12.3.1.14,8]nonadeca-1(18),4,6,8(19),14,16-hexaen-12-yl]cyclobutyl}carbamate (20.9 mg, 79%). ESI-MS m/z calc. 621.2621, found 622.5 (M+1)⁺; Retention time. 1.65 minutes; LC method A.

### Example 134: Preparation of Compound 169 and Compound 170

### Step 1: Methyl N-[3-[(11R)-6-(2,6-dimethylphenyl)-11-(2,2-dimethylpropyl)-2,2,13-trioxo-9-oxa-2),6-thia-3,5,12,19-tetrazatricyclo[12.3.1.14,8]nonadeca-1(18),4(19),5,7,14,16-hexaen-12-yl]cyclobutyl]-N-methyl-carbamate (stereoisomers Compound 169, diastereomer 1, and Compound 170, diastereomer 2)

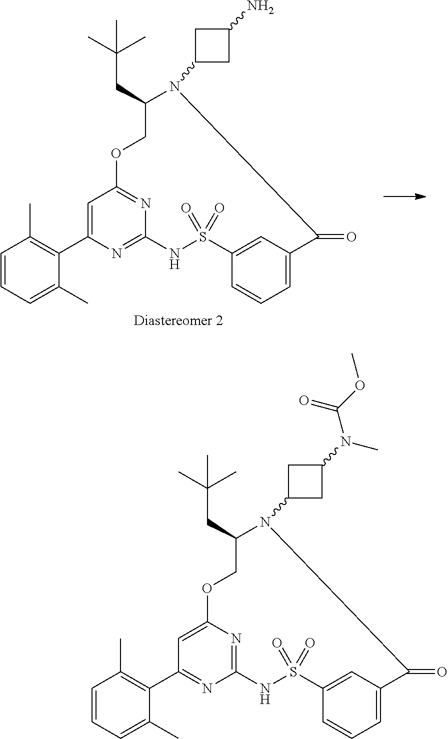 [SMILES: Cc1cccc(C)c1-c1cc2nc(n1)NS(=O)(=O)c1cccc(c1)C(=O)N(C1CC(N)C1)[C@H](CC(C)(C)C)CO2 ; COC(=O)N(C)C1CC(N2C(=O)c3cccc(c3)S(=O)(=O)Nc3nc(cc(-c4c(C)cccc4C)n3)OC[C@H]2CC(C)(C)C)C1]

(11R)-12-(3-Aminocyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (diastereomer 1, 25 mg, 0.03541 mmol) was combined with methyl chloroformate (approximately 6.692 mg, 5.472 μL, 0.07082 mmol) in DCM (0.5 mL) and DIPEA (approximately 22.88 mg, 30.84 μL, 0.1770 mmol) was added. The reaction was stirred for 10 minutes at room temperature, then was partitioned between 1M HCl and ethyl acetate. The layers were separated and the aqueous was extracted an additional 2× with ethyl acetate. The combined organics were washed with brine, dried over sodium sulfate and concentrated. The resulting white solid was used in the next step without further purification. The product was dissolved in THE (0.5 mL) and cooled to 0° C. in an ice bath. Sodium hydride (approximately 5.275 mg, 0.1319 mmol) was added, and after one minute the reaction was removed from the ice bath and allowed to stir at room temperature for 20 minutes. The reaction was then returned to the ice bath and methyl iodide (approximately 32.98 μL of 1 M, 0.03298 mmol) was added. The ice bath was removed again, and the reaction was allowed to stir for 3 hours at room temperature. After this time the reaction was partitioned between 1M HCl and ethyl acetate. The layers were separated and the aqueous was extracted an additional 2× with ethyl acetate. The combined organics were washed with brine, dried over sodium sulfate and concentrated. The resulting crude material was dissolved in 1:1 DMSO/methanol, filtered and purified by reverse phase HPLC (1-99% ACN in water, HCl modifier, 15 min run) to give as a white solid methyl N-[3-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]-N-methyl-carbamate (diastereomer 1, 10.7 mg, 47%). ESI-MS m/z calc. 635.2778, found 636.5 (M+1)⁺; Retention time: 1.81 minutes; LC method A. A separate reaction applying similar conditions to (11R)-12-(3-Aminocyclobutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (diastereomer 2, 15 mg, 0.02199 mmol) provided methyl N-[3-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]cyclobutyl]-N-methyl-carbamate (diastereomer 2, 7.1 mg, 50%). ESI-MS m/z calc. 635.2778, found 636.5 (M+1)⁺; Retention time: 1.82 minutes; LC method A.

### Example 135: Preparation of Compound 171

### Step 1: (11R)-12-(3-amino-3-methylcyclobutyl)-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione, diastereomer 1 and 2

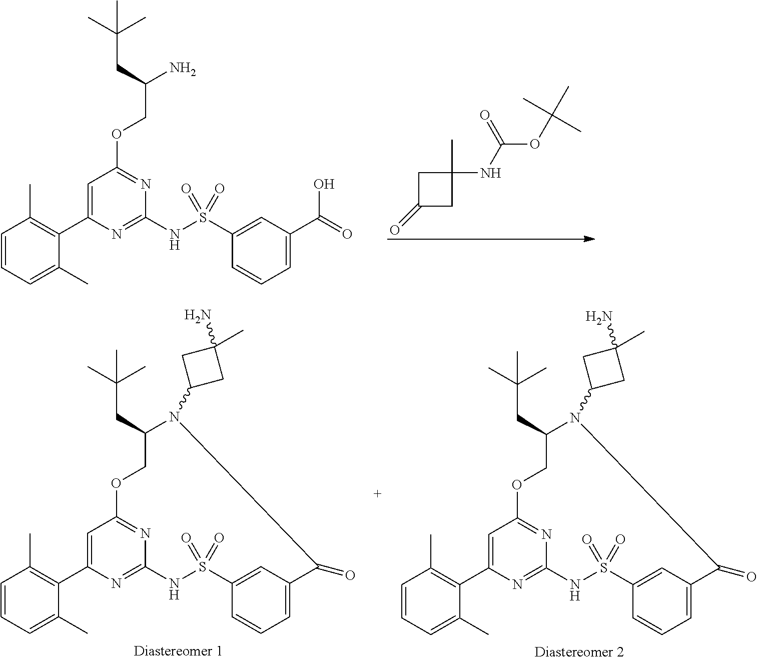 [SMILES: Cc1cccc(C)c1-c1cc(OC[C@H](N)CC(C)(C)C)nc(NS(=O)(=O)c2cccc(C(=O)O)c2)n1 ; CC1(NC(=O)OC(C)(C)C)CC(=O)C1 ; Cc1cccc(C)c1-c1cc2nc(n1)NS(=O)(=O)c1cccc(c1)C(=O)N(C1CC(C)(N)C1)[C@H](CC(C)(C)C)CO2 ; Cc1cccc(C)c1-c1cc2nc(n1)NS(=O)(=O)c1cccc(c1)C(=O)N(C1CC(C)(N)C1)[C@H](CC(C)(C)C)CO2]

3-[[4-[(2R)-2-amino-4,4-dimethyl-pentoxy]-6-(2,6-dimethylphenyl)pyrimidin-2-yl]sulfamoyl]benzoic acid (hydrochloride salt) (1.06 g, 1.930 mmol) was combined with tert-butyl N-(1-methyl-3-oxo-cyclobutyl)carbamate (500 mg, 2.509 mmol) in DCM (4 mL) and stirred at room temperature for 10 minutes. Sodium triacetoxyborohydride (1.25 g, 5.898 mmol) was added and the reaction was stirred at room temperature for 90 minutes. Conversion appeared to have stalled, and an additional portion of sodium triacetoxyborohydride (600 mg, 2.831 mmol) was added, along with tert-butyl N-(1-methyl-3-oxo-cyclobutyl)carbamate (200 mg, 1.004 mmol). The reaction was stirred for an additional two hours at room temperature, then was partitioned between 1M HCl and ethyl acetate (150 mL each). The layers were separated, and the aqueous was extracted an additional 2×100 mL ethyl acetate. The combined organics were washed with brine and dried over sodium sulfate. The resulting product was used in the next step without further purification (contains a significant amount of the N-linked rearranged side product). ESI-MS m/z calc. 695.33527, found 696.7 (M+1)⁺; Retention time: minutes; LC method D. 4

To a solution of the product from above in DMF (5 mL) was added dropwise to a stirring solution of COMU (1.7 g, 3.969 mmol) and DIPEA (2.1 mL, 12.06 mmol) in DMF (45 mL) over 5 minutes. The reaction mixture was allowed to stir for 16 hours at room temperature. It was then concentrated by rotary evaporation and the remaining residue was partitioned between 1 M HCl and ethyl acetate. The layers were separated and the aqueous was extracted an additional 3× with ethyl acetate. The combined organics were washed with brine, dried over sodium sulfate and concentrated. The resulting crude material was purified by chromatography on silica gel, eluting with a gradient of 0-100% ethyl acetate in hexanes. The fractions containing product were combined to give the macrolactamization product. ESI-MS m/z calc. 677.3247, found 678.7 (M+1)⁺; Retention time: 0.8 minutes; LC method D.

The product was dissolved in DCM (5 mL) and HCl (5 mL of 4 M, 20.00 mmol) and stirred at room temperature for 30 minutes. The reaction mixture was then concentrated by rotary evaporation. The resulting crude material was purified by reverse phase (1-99% Methanol in water, HCl modifier, shallow gradient in middle) to give two diastereomeric products separately, peak 1, diastereomer 1: (11R)-12-(3-amino-3-methylcyclobutyl)-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione (hydrochloride salt) (84 mg, 7%) ESI-MS m/z calc. 577.2723, found 578.7 (M+1)⁺; Retention time: 0.49 minutes (LC method D); and diastereomer 2, Peak 2: (11R)-12-(3-amino-3-methylcyclobutyl)-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione (hydrochloride salt) (114 mg, 10%) ESI-MS m/z calc. 577.2723, found 578.8 (M+1)⁺; Retention time: 0.52 minutes (LC method A).

### Step 2: methyl N-{3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(18),4,6,8(19),14,16-hexaen-12-yl]-1-methylcyclobutyl}carbamate (Compound 171)

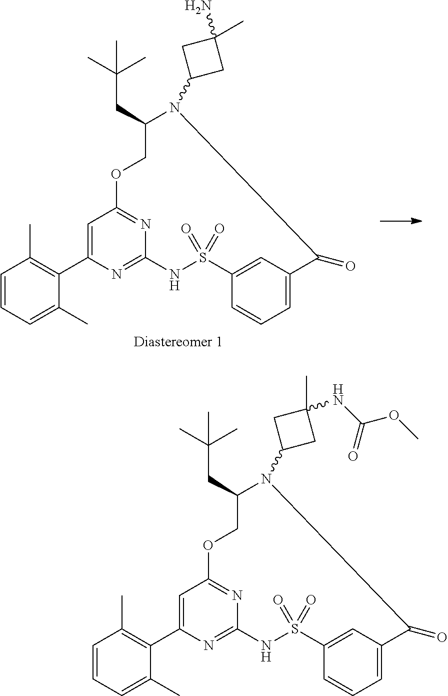 [SMILES: Cc1cccc(C)c1-c1cc2nc(n1)NS(=O)(=O)c1cccc(c1)C(=O)N(C1CC(C)(N)C1)[C@H](CC(C)(C)C)CO2 ; COC(=O)NC1(C)CC(N2C(=O)c3cccc(c3)S(=O)(=O)Nc3nc(cc(-c4c(C)cccc4C)n3)OC[C@H]2CC(C)(C)C)C1]

(11R)-12-(3-amino-3-methylcyclobutyl)-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione (hydrochloride salt), diastereomer 1, (38 mg, 0.06187 mmol) was combined in DCM with methyl chloroformate (approximately 11.69 mg, 9.558 μL, 0.1237 mmol) and DIPEA (approximately 39.99 mg, 53.89 μL, 0.3094 mmol), and stirred at room temperature for 30 minutes. After this time the reaction mixture was quenched with several drops of aqueous 1M HCl. The reaction mixture was then partially concentrated, diluted with 1:1 DMSO and methanol, filtered, and purified by reverse phase HPLC (1-99% ACN in water, HCl modifier, 15 min run) to give upon drying methyl N-{3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(18),4,6,8(19),14,16-hexaen-12-yl]-1-methylcyclobutyl}carbamate (24.9 mg, 63%). ESI-MS m/z calc. 635.2778, found 636.5 (M+1)⁺; Retention time: 1.73 minutes; LC method A. ¹H NMR (400 MHz, DMSO-d₆) δ 13.08 (s, 1H), 8.44 (s, 1H), 7.92 (s, 1H), 7.67 (s, 2H), 7.28 (d, J=26.5 Hz, 2H), 7.11 (s, 2H), 6.40 (s, 1H), 5.10 (d, J=9.7 Hz, 1H), 4.24 (s, 1H), 4.02 (t, J=8.9 Hz, 1H), 3.69 (s, 1H), 3.54 (s, 3H), 2.94 (dt, J=28.9, 10.2 Hz, 2H), 2.46 (d, J=7.3 Hz, 2H), 2.32-1.70 (m, 6H), 1.69-1.52 (m, 1H), 1.45 (s, 3H), 1.38 (d, J=14.9 Hz, 1H), 0.49 (s, 9H).

### Example 136: Preparation of Compound 172

### Step 1: Methyl N-{3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(18),4,6,8(19),14,16-hexaen-12-yl]-1-methylcyclobutyl}carbamate (Compound 172)

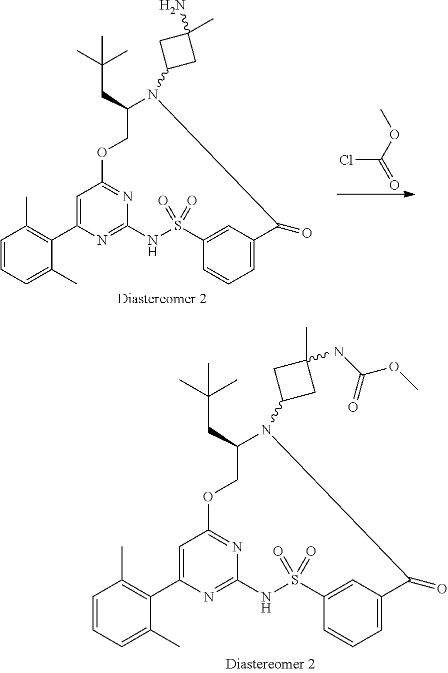

(11R)-12-(3-amino-3-methylcyclobutyl)-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione, diastereomer 2 (hydrochloride salt)(36 mg, 0.05861 mmol) was combined in DM with methyl chloroformate (approximately 11.08 mg, 9.060 μL, 0.1172 mmol) and DIPEA (approximately 37.87 mg, 51.04 μL, 0.2930 mmol), and stirred at room temperature for 30 minutes. After this time the reaction mixture was quenched with several drops of aqueous 1M HCl. The reaction mixture was then partially concentrated, diluted with 1:1 DMSO and methanol, filtered, and purified by prep HPLC (1-99% ACN in water, HCl modifier, 15 minute run) to give upon drying methyl N-{3-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(18),4,6,8(19),14,16-hexaen-12-yl]-1-methylcyclobutyl}carbamate (20.5 mg, 54%). ESI-MS m/z calc. 635.2778, found 636.6 (M+1)⁺; Retention time: 1.77 minutes (LC method A).

### Example 137: Preparation of Compound 173

### Step 1: methyl N-[3-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]-1-methyl-cyclobutyl]-N-methyl-carbamate (Compound 173)

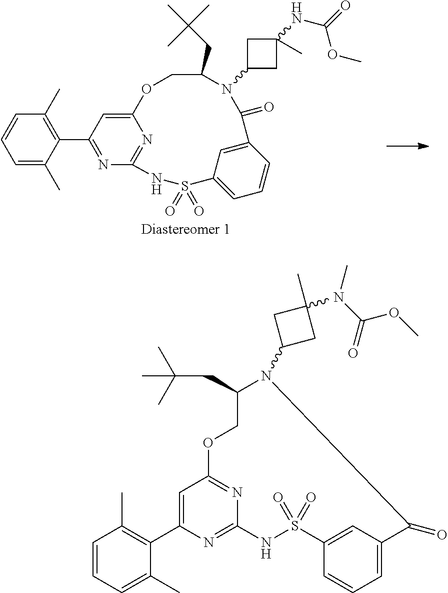 [SMILES: COC(=O)NC1(C)CC(N2C(=O)c3cccc(c3)S(=O)(=O)Nc3nc(cc(-c4c(C)cccc4C)n3)OC[C@H]2CC(C)(C)C)C1 ; COC(=O)N(C)C1(C)CC(N2C(=O)c3cccc(c3)S(=O)(=O)Nc3nc(cc(-c4c(C)cccc4C)n3)OC[C@H]2CC(C)(C)C)C1]

N-{3-[(1R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(18),4,6,8(19),14,16-hexaen-12-yl]-1-methylcyclobutyl}carbamate (15 mg, 0.02359 mmol) in THE was added to NaH (approximately 4.720 mg, 0.1180 mmol) in a screwcap vial. The reaction mixture was stirred for 20 minutes at room temperature then was cooled to 0° C. Iodomethane (approximately 47.18 μL of 1 M, 0.04718 mmol) was added as a solution in THF. The reaction mixture was then returned to room temperature for one hour and then heated to 40° C. After four total additional hours, the reaction mixture was partitioned between 1 M HCl and ethyl acetate. The layers were separated and the aqueous was extracted an additional time with ethyl acetate. The combined organics were dried over sodium sulfate and concentrated, then dissolved in 1:1 DMSO/methanol and filtered then purified by reverse phase HPLC (1-99% ACN in water, HCl modifier, 15 min run) to give methyl N-[3-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]-1-methyl-cyclobutyl]-N-methyl-carbamate as a white powder upon drying (6.4 mg, 41%). ESI-MS m/z calc. 649.2934, found 650.5 (M+1)⁺; Retention time: 1.87 minutes; LC method A.

### Example 138: Preparation of Compound 174, Compound 175, and Compound 176

### Step 1: Ethyl 1-(tert-butoxycarbonylamino)-3-[[(1R)-1-(hydroxymethyl)-3,3-dimethyl-butyl]amino]cyclobutanecarboxylate

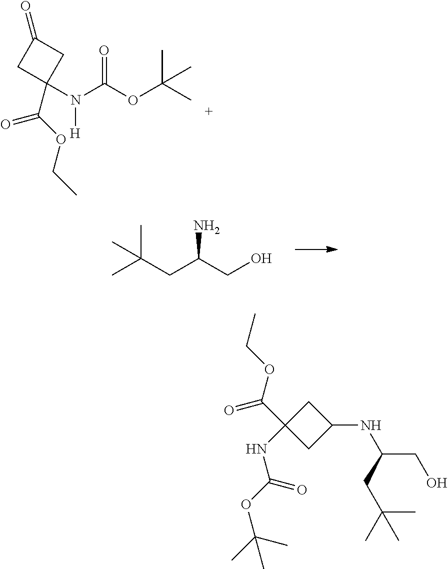

Ethyl 1-(tert-butoxycarbonylamino)-3-oxo-cyclobutanecarboxylate (300 mg, 1.166 mmol) was added to a solution of (2R)-2-amino-4,4-dimethyl-pentan-1-ol (hydrochloride salt)(240 mg, 1.431 mmol) in anhydrous dichloromethane (2 mL) and stirred at room temperature for 30 minutes. sodium triacetoxyborohydride (790 mg, 3.727 mmol) was added and the reaction was stirred an additional 3 hours, then placed in a refrigerator for 16 hours. The reaction mixture was then allowed to warm to room temperature, diluted with 5 mL dichloromethane and aqueous HCl (5 mL of 1 M, 5.000 mmol) was added slowly and the reaction was stirred at room temperature an additional 10 minutes. A solution of potassium carbonate (2 g, 14.47 mmol) in water (2.5 mL) was added and the organics were separated. The aqueous was extracted with DCM then ethyl acetate, and the combined organics were washed with brine and dried over sodium sulfate to give as a colorless oil after concentration, ethyl 1-(tert-butoxycarbonylamino)-3-[[(1R)-1-(hydroxymethyl)-3,3-dimethyl-butyl]amino]cyclobutanecarboxylate (393 mg, 90%). ESI-MS m/z calc. 372.26242, found 373.7 (M+1)⁺; Retention time: 0.46 minutes; (LC method D).

### Step 2: tert-Butyl N-[1-(Hydroxymethyl)-3-[[(1R)-1-(hydroxymethyl)-3,3-dimethyl-butyl]amino]cyclobutyl]carbamate

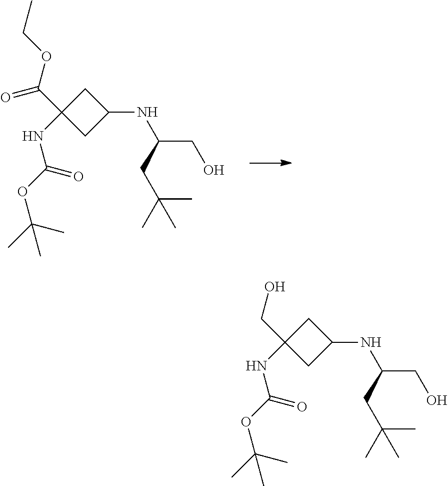 [SMILES: CCOC(=O)C1(NC(=O)OC(C)(C)C)CC(N[C@@H](CO)CC(C)(C)C)C1 ; CC(C)(C)C[C@H](CO)NC1CC(CO)(NC(=O)OC(C)(C)C)C1]

Ethyl 1-(tert-butoxycarbonylamino)-3-[[(1R)-1-(hydroxymethyl)-3,3-dimethyl-butyl]amino]cyclobutanecarboxylate (393 mg, 1.055 mmol) was combined with lithium borohydride (6 mL of 2 M, 12.00 mmol) in THE (5 mL), and stirred at room temperature for 6 hours. The reaction was cooled to 0° C., and methanol (3 mL, 74.06 mmol) and water (3 mL) were added and the reaction was stirred for 15 minutes (minor bubbling)). Saturated aqueous ammonium chloride was added dropwise, and the reaction mixture began bubbling vigorously. Additional saturated ammonium chloride was added until bubbling ceased, and the reaction was stirred at room temperature for a further 20 minutes. The reaction mixture was then diluted with water and ethyl acetate, and the layers were separated. The aqueous was extracted an additional 3× with ethyl acetate, and the combined organics were washed with brine, dried over sodium sulfate, and concentrated to give as a white solid tert-butyl N-[1-(hydroxymethyl)-3-[[(1R)-1-(hydroxymethyl)-3,3-dimethyl-butyl]amino]cyclobutyl]carbamate (310 mg, 89%) E ESI-MS m z calc. 330.25186, found 331.3 (M+1)⁺; Retention time: 0.43 minutes. ESI-MS m/z calc. 330.25186, found 331.3 (M+1)⁺; Retention time: 0.43 minutes; (LC method D).

### Step 3: (11R)-6-(2,6-dimethylphenyl)-11-(2,2-dimethylpropyl)-12-{6-oxo-7-oxa-5-azaspiro[3.4]octan-2-yl}-9-oxa-2λ⁶-thia-3,5,12,19-tetraazatricyclo[12.3.1.14,8]nonadeca-1(17),4(19),5,7,14(18),15-hexaene-2,2,13-trione, diastereomer 1 (Compound 175), and (11R)-6-(2,6-dimethylphenyl)-11-(2,2-dimethylpropyl)-12-{6-oxo-7-oxa-5-azaspiro[3.4]octan-2-yl}-9-oxa-2λ⁶-thia-3,5,12,19-tetraazatricyclo[12.3.1.14,8]nonadeca-1(17),4(19),5,7,14(18),15-hexaene-2,2,13-trione, diastereomer 2 (Compound 176)

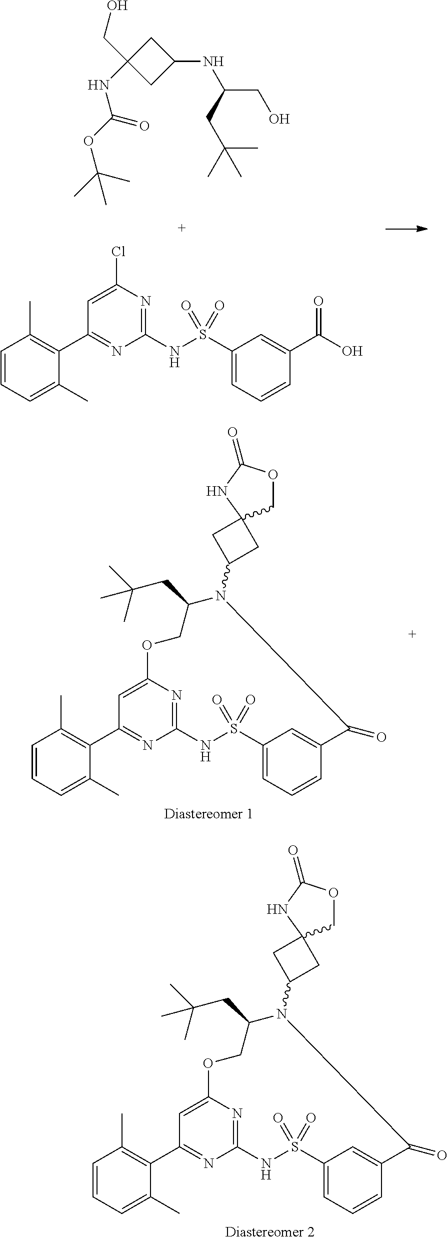

tert-Butyl N-[1-(hydroxymethyl)-3-[[(1R)-1-(hydroxymethyl)-3,3-dimethyl-butyl]amino]cyclobutyl]carbamate (310 mg, 0.9381 mmol) was combined with sodium tert-butoxide (535 mg, 5.567 mmol) in THE (9 mL) and stirred at 50° C. for 30 minutes. The reaction mixture was then removed from heat and 3-[[4-chloro-6-(2,6-dimethylphenyl)pyrimidin-2-yl]sulfamoyl]benzoic acid (350 mg, 0.8376 mmol) and an additional sodium tert-butoxide (250 mg, 2.601 mmol) were added and the reaction was returned to 50° C. for 1 hour. The reaction mixture was cooled to room temperature and partitioned between 1M HCl and ethyl acetate. The layers were separated, and the aqueous was extracted 4× with ethyl acetate. The combined organics were washed with brine, dried over sodium sulfate and concentrated. This material was purified by prep HPLC (1-70% ACN in water, HCl modifier, 15 minute run) to give 3-[[4-[(2R)-4,4-dimethyl-2-[(6-oxo-7-oxa-5-azaspiro[3.4]octan-2-yl)amino]pentoxy]-6-(2,6-dimethylphenyl)pyrimidin-2-yl]sulfamoyl]benzoic acid (166.0 mg, 28%). A solution of that product in DMF (5 mL) was added dropwise over 5 minutes to a stirring solution of COMU (300 mg, 0.7005 mmol) and DIPEA (350 μL, 2.009 mmol) in DMF (15 mL). The reaction mixture was stirred for an additional 3 hours at room temperature, then was partitioned between 1M HCl and ethyl acetate. The layers were separated and the aqueous was extracted an additional 3× with ethyl acetate. The combined organics were washed with brine, dried over sodium sulfate and concentrated. The resulting crude material was dissolved in 1:1 DMSO/methanol, filtered, and purified by reverse phase chromatography (1-99% MeOH in water/HCl modifier) to give two stereoisomers of the spirocycle separately, peak 1, diastereomer 1, (11R)-6-(2,6-dimethylphenyl)-11-(2,2-dimethylpropyl)-12-{6-oxo-7-oxa-5-azaspiro[3.4]octan-2-yl}-9-oxa-2λ⁶-thia-3,5,12,19-tetraazatricyclo[12.3.1.14,8]nonadeca-1(17),4(19),5,7,14(18),15-hexaene-2,2,13-trione (45 mg, 8%) ESI-MS m/z calc. 619.24646, found 620.6 (M+1)⁺; Retention time: 1.51 minutes (LC method A); and peak 2, diastereomer 2, (11R)-6-(2,6-dimethylphenyl)-11-(2,2-dimethylpropyl)-12-{6-oxo-7-oxa-5-azaspiro[3.4]octan-2-yl}-9-oxa-2λ⁶-thia-3,5,12,19-tetraazatricyclo[12.3.1.14,8]nonadeca-1(17),4(19),5,7,14(18),15-hexaene-2,2,13-trione (19 mg, 3%) ESI-MS m/z calc. 619.24646, found 620.5 (M+1)⁺; Retention time: 1.59 minutes (LC method A).

### Step 4: (11R)-6-(2,6-Dimethylphenyl)-11-(2,2-dimethylpropyl)-12-(5-methyl-6-oxo-7-oxa-5-azaspiro[3.4]octan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one (compound 174)

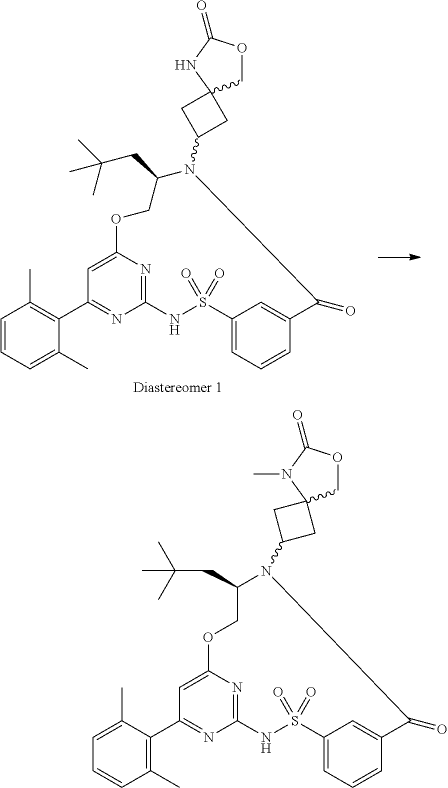 [SMILES: Cc1cccc(C)c1-c1cc2nc(n1)NS(=O)(=O)c1cccc(c1)C(=O)N(C1CC3(COC(=O)N3)C1)[C@H](CC(C)(C)C)CO2 ; Cc1cccc(C)c1-c1cc2nc(n1)NS(=O)(=O)c1cccc(c1)C(=O)N(C1CC3(COC(=O)N3C)C1)[C@H](CC(C)(C)C)CO2]

(11R)-6-(2,6-Dimethylphenyl)-11-(2,2-dimethylpropyl)-12-{6-oxo-7-oxa-5-azaspiro[3.4]octan-2-yl}-9-oxa-2λ⁶-thia-3,5,12,19-tetraazatricyclo[12.3.1.14,8]nonadeca-1(17),4(19),5,7,14(18),15-hexaene-2,2,13-trione, diastereomer 1 (10 mg, 0.01597 mmol) in THF was added to NaH (approximately 3.194 mg, 0.07985 mmol) in a screwcap vial. The reaction mixture was stirred for 20 minutes at room temperature then was cooled to 0° C. iodomethane (approximately 31.94 μL of 1 M, 0.03194 mmol) was added as a solution in THF. The reaction mixture was then returned to room temperature for one hour. The reaction was then heated to 40° C. Additional iodomethane (approximately 31.94 μL of 1 M, 0.03194 mmol) was added after 1 hour at 40° C. After four total additional hours, the reaction mixture was each partitioned between 1M HCl and ethyl acetate. The layers were separated and the aqueous was extracted an additional time with ethyl acetate. The combined organics were dried over sodium sulfate and concentrated, then dissolved in 1:1 DMSO/methanol and filtered then purified by reverse phase HPLC (1-99% ACN in water, HCl modifier, 15 min run) to give (11R)-6-(2,6-dimethylphenyl)-11-(2,2-dimethylpropyl)-12-(5-methyl-6-oxo-7-oxa-5-azaspiro[3.4]octan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one as a white powder upon drying (3.7 mg, 36%). ESI-MS m/z calc. 633.2621, found 634.6 (M+1)⁺; Retention time: 1.62 minutes; LC method A.

### Example 139: Preparation of Compound 177

### Step 1: (11R)-6-(2,6-dimethylphenyl)-11-(2,2-dimethylpropyl)-12-(5-methyl-6-oxo-7-oxa-5-azaspiro[3.4]octan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 177)

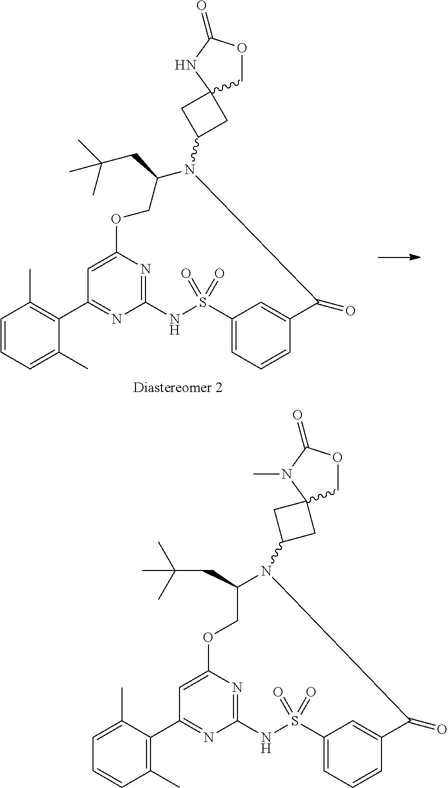 [SMILES: Cc1cccc(C)c1-c1cc2nc(n1)NS(=O)(=O)c1cccc(c1)C(=O)N(C1CC3(COC(=O)N3)C1)[C@H](CC(C)(C)C)CO2 ; Cc1cccc(C)c1-c1cc2nc(n1)NS(=O)(=O)c1cccc(c1)C(=O)N(C1CC3(COC(=O)N3C)C1)[C@H](CC(C)(C)C)CO2]

(11R)-6-(2,6-dimethylphenyl)-11-(2,2-dimethylpropyl)-12-{6-oxo-7-oxa-5-azaspiro[3.4]octan-2-yl}-9-oxa-2λ⁶-thia-3,5,12,19-tetraazatricyclo[12.3.1.14,8]nonadeca-1(17),4(19),5,7,14(18),15-hexaene-2,2,13-trione, diastereomer 2, (10 mg, 0.01597 mmol) in THE was added to NaH (approximately 3.194 mg, 0.07985 mmol) in a screwcap vial. The reaction mixture was stirred for 20 minutes at room temperature then was cooled to 0° C. iodomethane (approximately 31.94 μL of 1 M, 0.03194 mmol) was added as a solution in THF. The reaction mixture was then returned to room temperature for one hour. The reaction was then heated to 40° C. Additional iodomethane (approximately 31.94 μL of 1 M, 0.03194 mmol) was added after 1 hour at 40° C. After four total additional hours, the reaction mixture was partitioned between 1M HCl and ethyl acetate. The layers were separated and the aqueous was extracted an additional time with ethyl acetate. The combined organics were dried over sodium sulfate and concentrated, then dissolved in 1:1 DMSO/methanol and filtered then purified by reverse phase HPLC (1-99% ACN in water, HCl modifier, 15 min run) to give (11R)-6-(2,6-dimethylphenyl)-11-(2,2-dimethylpropyl)-12-(5-methyl-6-oxo-7-oxa-5-azaspiro[3.4]octan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one as a white powder upon drying (3.8 mg, 36%). ESI-MS m/z calc. 633.2621, found 634.4 (M+1)⁺; Retention time: 1.63 minutes; LC method A.

### Example 140: Preparation of Compound 178

### Step 1: (11R)-6-(2,6-Dimethylphenyl)-11-(2,2-dimethylpropyl)-12-(3-morpholinocyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 178)

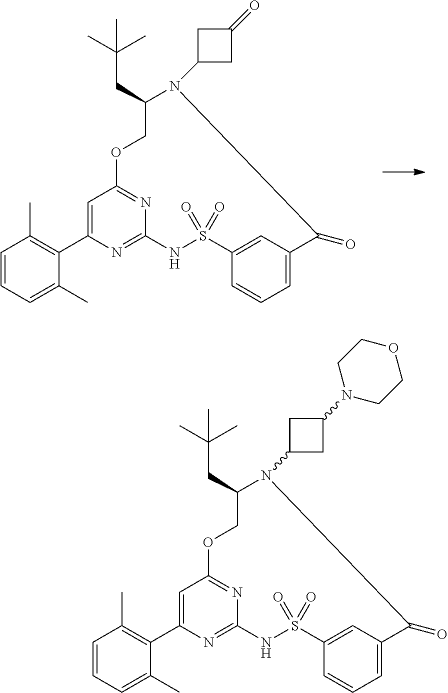 [SMILES: Cc1cccc(C)c1-c1cc2nc(n1)NS(=O)(=O)c1cccc(c1)C(=O)N(C1CC(=O)C1)[C@H](CC(C)(C)C)CO2 ; Cc1cccc(C)c1-c1cc2nc(n1)NS(=O)(=O)c1cccc(c1)C(=O)N(C1CC(N3CCOCC3)C1)[C@H](CC(C)(C)C)CO2]

(11R)-6-(2,6-Dimethylphenyl)-11-(2,2-dimethylpropyl)-2,2-dioxo-12-(3-oxocyclobutyl)-9-oxa-2λ⁶-thia-3,5,12,19-tetrazatricyclo[12.3.1.14,8]nonadeca-1(18),4(19),5,7,14,16-hexaen-13-one (50 mg, 0.08886 mmol) and morpholine (16 mg, 0.1837 mmol) were combined and dissolved in dichloromethane (0.50 mL). The mixture was stirred at room temperature for 15 minutes. Sodium triacetoxyborohydride (57 mg, 0.2689 mmol) was then added, and the reaction mixture was allowed to stir overnight at room temperature. The reaction mixture was filtered, and the product was isolated by UV-triggered reverse phase HIPLC eluting with a 10-99% acetonitrile/water gradient over 15 minutes with 0.5 mM HCl acid modifier in the aqueous phase. (11R)-6-(2,6-dimethylphenyl)-11-(2,2-dimethylpropyl)-12-(3-morpholinocyclobutyl)-2,2-dioxo-9-oxa-2λ⁶-thia-3,5,12,19-tetrazatricyclo[12.3.1.14,8]nonadeca-1(18),4(19),5,7,14,16-hexaen-13-one (41.5 mg, 740%) was obtained. ESI-MS m/z calc. 633.29846, found 634.3 (M+1)⁺; Retention time: 1.29 minutes; LC method A.

### Example 141: Preparation of Compound 179

### Step 1: 3-[[4-[(1R)-2-Amino-1-methyl-ethoxy]-6-(2,6-dimethylphenyl)pyrimidin-2-yl]sulfamoyl]benzoic acid

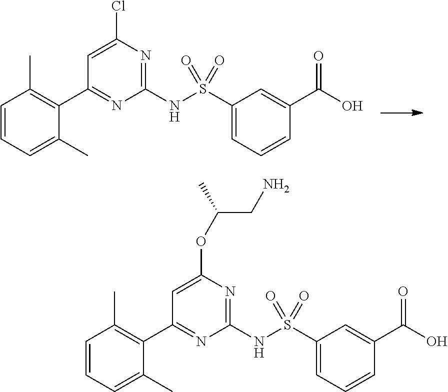

3-[[4-Chloro-6-(2,6-dimethylphenyl)pyrimidin-2-yl]sulfamoyl]benzoic acid (3 g, 7.179 mmol) and (2R)-1-aminopropan-2-ol (885 mg, 11.78 mmol) were combined in THF (18 mL), and sodium tert-butoxide (2.76 g, 28.72 mmol) was added. The reaction mixture became warm to the touch and was stirred for 15 minutes without any additional heating. The reaction mixture was then added to a separatory funnel containing 1M HCl and ethyl acetate. The layers were separated and the aqueous was extracted 3 additional times with ethyl acetate. The combined organics were washed with brine. Some product appeared to begin crashing out of solution at this point and the brine layer was extracted an additional time with ethyl acetate. The combined organics were dried over sodium sulfate, filtered and concentrated to give a white solid, 3-[[4-[(1R)-2-amino-1-methyl-ethoxy]-6-(2,6-dimethylphenyl)pyrimidin-2-yl]sulfamoyl]benzoic acid (hydrochloride salt) (2.1 g, 59%) ESI-MS m/z calc. 456.14673, found 457.1 (M+1)⁺; Retention time: 0.37 minutes; (LC method D). ¹H NMR (500 MHz, DMSO) δ 8.45 (d, J=2.4 Hz, 1H), 8.22 (s, 2H), 8.14 (dd, J=13.6, 7.8 Hz, 2H), 7.72 (t, J=7.8 Hz, 1H), 7.27 (t, J=7.7 Hz, 1H), 7.14 (d, J=7.5 Hz, 2H), 6.27 (s, 1H), 5.11 (t, J=7.5 Hz, 1H), 3.19 (d, J=13.2 Hz, 1H), 3.08 (t, J=11.1 Hz, 1H), 2.02 (d, J=20.9 Hz, 6H), 1.30 (d, J=6.2 Hz, 3H).

### Step 2: tert-Butyl 2-[(10R)-6-(2,6-dimethylphenyl)-10-methyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate

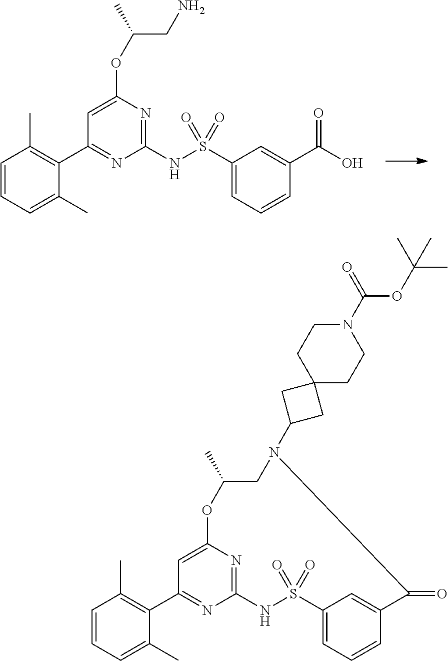 [SMILES: Cc1cccc(C)c1-c1cc(O[C@H](C)CN)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CCN(C(=O)OC(C)(C)C)CC3)C1)C[C@@H](C)O2]

3-[[4-[(1R)-2-amino-1-methyl-ethoxy]-6-(2,6-dimethylphenyl)pyrimidin-2-yl]sulfamoyl]benzoic acid (100 mg, 0.2191 mmol) was combined with the corresponding tert-butyl 2-oxo-7-azaspiro[3.5]nonane-7-carboxylate (approximately 104.9 mg, 0.4382 mmol) in dichloromethane (1 mL) and stirred at room temperature. After 5 minutes sodium triacetoxyborohydride (approximately 139.3 mg, 0.6573 mmol) was added and the reaction mixture was stirred for 30 minutes at room temperature. An additional portion of sodium triacetoxyborohydride was added and the reaction was stirred for an additional 2 h at room temperature. The reaction mixture was then partitioned between 1M HCl and ethyl acetate. The layers were separated and the aqueous was extracted an additional 4×ethyl acetate. The combined organics were dried over sodium sulfate and concentrated. The resulting crude material was used in the next step without further purification. The product was combined with HATU (approximately 108.3 mg, 0.2848 mmol) in DMF (25 mL) and DIPEA (approximately 141.7 mg, 191.0 μL, 1.096 mmol) was added and the reactions were stirred for 6 hours at room temperature. The reaction mixture was partitioned between 1M HCl and ethyl acetate and the layers were separated. The aqueous was extracted twice with ethyl acetate and the combined organics were washed with water and brine and dried over sodium sulfate and concentrated. The resulting crude was purified by column chromatography on silica gel eluting with 0-100% ethyl acetate in dichloromethane) to give tert-butyl 2-[(10R)-6-(2,6-dimethylphenyl)-10-methyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (40 mg, 28%). ESI-MS m/z calc. 661.2934, found 662.3 (M+1)⁺; Retention time: 0.74 minutes; LC method D.

### Step 3: (10R)-12-(7-Azaspiro[3.5]nonan-2-yl)-6-(2,6-dimethylphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one

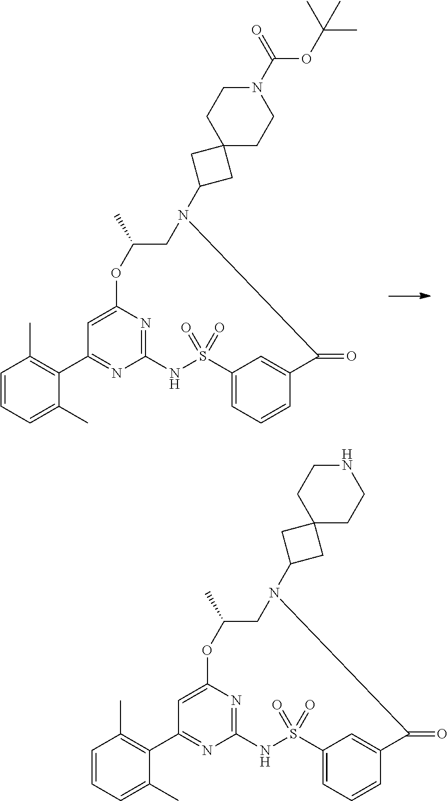 [SMILES: Cc1cccc(C)c1-c1cc2nc(n1)NS(=O)(=O)c1cccc(c1)C(=O)N(C1CC3(CCN(C(=O)OC(C)(C)C)CC3)C1)C[C@@H](C)O2 ; Cc1cccc(C)c1-c1cc2nc(n1)NS(=O)(=O)c1cccc(c1)C(=O)N(C1CC3(CCNCC3)C1)C[C@@H](C)O2]

tert-Butyl 2-[(10R)-6-(2,6-dimethylphenyl)-10-methyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (40 mg, 0.06044 mmol) was dissolved in DCM (0.3 mL) and HCl (300 μL of 4 M, 1.200 mmol) was added. The reaction was stirred at room temperature for 30 minutes then concentrated. Hexanes were added and the reaction mixture was concentrated a second time to give a white solid, which was used in the next step without further purification, (10R)-12-(7-azaspiro[3.5]nonan-2-yl)-6-(2,6-dimethylphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (36 mg, 100%). ESI-MS m/z calc. 561.24097, found 562.5 (M+1)⁺; Retention time: 0.43 minutes; (LC method D).

### Step 4: (10R)-6-(2,6-Dimethylphenyl)-10-methyl-12-(7-methyl-7-azaspiro[3.5]nonan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 179)

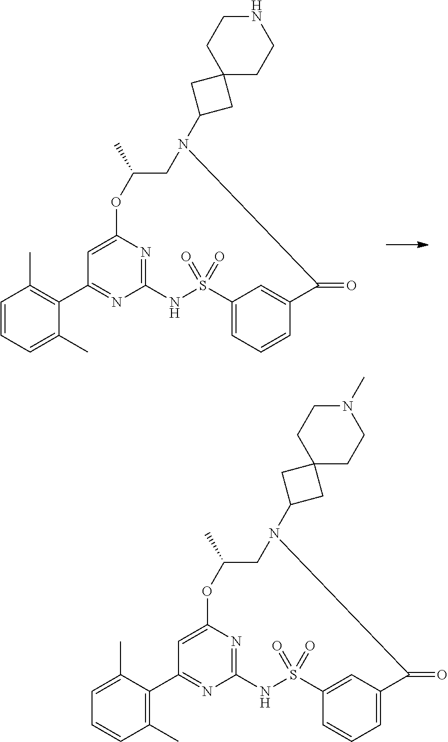 [SMILES: Cc1cccc(C)c1-c1cc2nc(n1)NS(=O)(=O)c1cccc(c1)C(=O)N(C1CC3(CCNCC3)C1)C[C@@H](C)O2 ; Cc1cccc(C)c1-c1cc2nc(n1)NS(=O)(=O)c1cccc(c1)C(=O)N(C1CC3(CCN(C)CC3)C1)C[C@@H](C)O2]

(10R)-12-(7-Azaspiro[3.5]nonan-2-yl)-6-(2,6-dimethylphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (22 mg, 0.03678 mmol) was combined with formic acid (0.2 mL) and aqueous Formaldehyde (0.3 mL, 10.89 mmol) and heated to 95° C. in a screwcap vial for 16 hours. After cooling to room temperature the reaction mixture was partially concentrated, filtered, diluted with methanol and purified by reverse phase HPLC (1-70% ACN in water, HCl modifier, 15 min run) to give as a white solid (10R)-6-(2,6-dimethylphenyl)-10-methyl-12-(7-methyl-7-azaspiro[3.5]nonan-2-yl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (4.1 mg, 18%) ESI-MS m/z calc. 575.25665, found 576.5 (M+1)⁺; Retention time: 1.03 minutes; LC method A.

### Example 142: Preparation of Compound 180

### Step 1: (10R)-6-(2,6-Dimethylphenyl)-10-methyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (Compound 180)

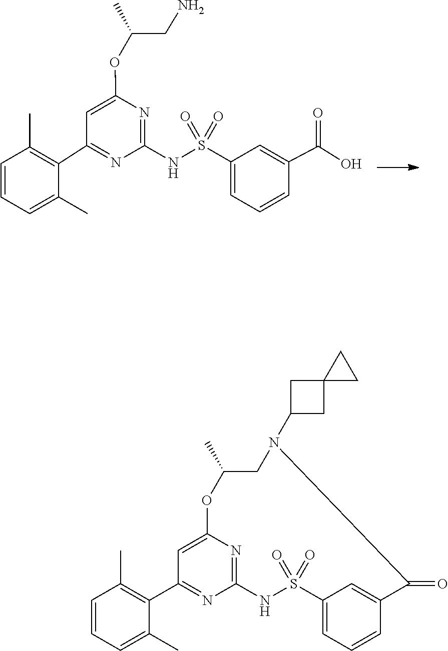 [SMILES: Cc1cccc(C)c1-c1cc(O[C@H](C)CN)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CC3)C1)C[C@@H](C)O2]

3-[[4-[(1R)-2-amino-1-methyl-ethoxy]-6-(2,6-dimethylphenyl)pyrimidin-2-yl]sulfamoyl]benzoic acid (40 mg, 0.08762 mmol) was combined with the spiro[2.3]hexan-5-one (approximately 16.84 mg, 0.1752 mmol) compound in dichloromethane (0.3 mL) at room temperature. After stirring for 5 minutes at room temperature sodium triacetoxyborohydride (approximately 55.72 mg, 0.2629 mmol) was added. After 30 minutes a second portion of sodium triacetoxyborohydride (approximately 55.72 mg, 0.2629 mmol) was added and the reaction mixture was stirred for an additional hour at room temperature. The reaction mixture was then partitioned between 1M HCl and ethyl acetate. The layers were separated, and the aqueous layer was extracted an additional three times with ethyl acetate. The combined organics were washed with brine, dried over sodium sulfate, and concentrated. The resulting reductive amination products were used in the next step without further purification. The product was dissolved in DMF (2 mL) and added dropwise over 5 minutes to a stirring solution of HATU (approximately 43.31 mg, 0.1139 mmol) and DIPEA (approximately 56.62 mg, 76.31 μL, 0.4381 mmol) in DMF (10 mL). The reaction mixture was allowed to stir at room temperature for an hour after the completion of addition, then was concentrated to a volume of 1 mL, filtered and purified by reverse phase HPLC to give the (10R)-6-(2,6-dimethylphenyl)-10-methyl-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (2.9 mg, 6%). ESI-MS m/z calc. 518.1988, found 519.3 (M+1)⁺; Retention time: 1.76 minutes; LC method A.

### Example 143: Preparation of Compound 181

### Step 1: Isopropyl 2-[(10R)-6-(2,6-dimethylphenyl)-10-methyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (Compound 181)

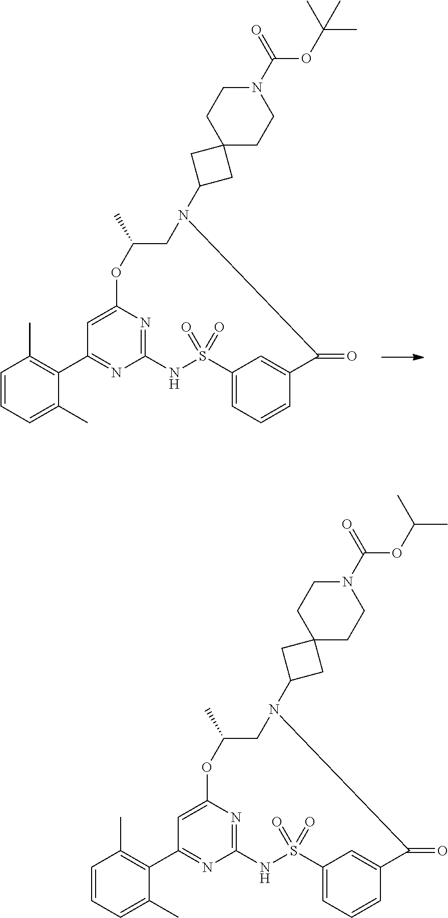 [SMILES: Cc1cccc(C)c1-c1cc2nc(n1)NS(=O)(=O)c1cccc(c1)C(=O)N(C1CC3(CCN(C(=O)OC(C)(C)C)CC3)C1)C[C@@H](C)O2 ; Cc1cccc(C)c1-c1cc2nc(n1)NS(=O)(=O)c1cccc(c1)C(=O)N(C1CC3(CCN(C(=O)OC(C)C)CC3)C1)C[C@@H](C)O2]

tert-Butyl 2-[(10R)-6-(2,6-dimethylphenyl)-10-methyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (21 mg, 0.03173 mmol) (several major impurities present in SM) was dissolved in dichloromethane (0.5 mL), and HCl (300 μL of 4 M, 1.200 mmol) was added. The reaction mixture was stirred for 20 minutes at room temperature and was then concentrated to a solid residue. Hexanes and a small amount of dichloromethane were added to the resulting residue and evaporated to give a white solid, which was used in the second step without further purification: (10R)-12-(7-azaspiro[3.5]nonan-2-yl)-6-(2,6-dimethylphenyl)-10-methyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) ESI-MS m/z calc. 561.24097, found 562.4 (M+1)⁺; Retention time: 0.42 minutes; (LC method D).

The product was dissolved in dichloromethane (0.5 mL) and triethylamine (25 μL, 0.1794 mmol) was added, followed by isopropyl chloroformate (30 μL of 2 M, 0.06000 mmol). The reaction was stirred for 5 minutes at room temperature, then was quenched with three drops of 1M HCl. The reaction mixture was partially concentrated then was dissolved in 1:1 methanol/DMSO, filtered and purified by reverse phase HPLC (1-99% ACN in water, HCl modifier, 30 min run). A close eluting impurity partially overlapped and the product was further purified with a second reverse phase HPLC run at (1-70% ACN in water, HCl modifier, 30 minutes) to give isopropyl 2-[(10R)-6-(2,6-dimethylphenyl)-10-methyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-7-azaspiro[3.5]nonane-7-carboxylate (4.2 mg, 20%) ESI-MS m/z calc. 647.2778, found 648.5 (M+1)⁺; Retention time: 1.82 minutes; (LC method A).

### Example 144: Preparation of Compound 182

### Step 1: (2R)-4,4-Dimethyl-2-(spiro[2.3]hexan-5-ylamino)pentan-1-ol

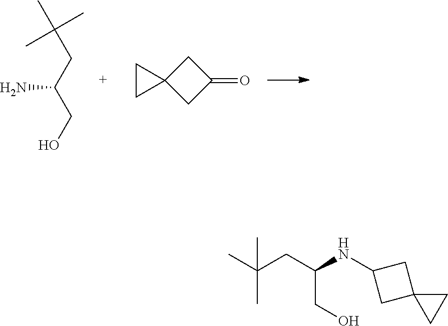

(2R)-2-Amino-4,4-dimethyl-pentan-1-ol (hydrochloride salt) (504 mg, 3.0058 mmol) was dissolved in DCE (10 mL) and spiro[2.3]hexan-5-one (321 mg, 3.3393 mmol) was added. The mixture was stirred at room temperature for 30 minutes, then sodium triacetoxyborohydride (1.92 g, 9.0591 mmol) was added in 3 batches. The mixture was allowed to stir overnight and then 1M HCl was added until a pH of ˜1 was achieved. The mixture was stirred vigorously for 20 min. The layers were separated, and the aqueous layer was extracted twice with DCM (10 mL). The organic layers were dried over sodium sulfate and concentrated to afford (2R)-4,4-dimethyl-2-(spiro[2.3]hexan-5-ylamino)pentan-1-ol (570 mg, 90%) as a viscous colorless oil. ¹H NMR (250 MHz, DMSO) δ 4.51 (t, J=5.5 Hz, 1H), 3.53-3.41 (m, 1H), 3.35-3.25 (m, 1H), 3.19-3.03 (m, 1H), 2.18-2.01 (m, 2H), 2.02-1.85 (m, 2H), 1.14 (d, J=4.8 Hz, 2H), 0.89 (s, 9H), 0.47-0.27 (m, 4H).

### Step 2: 3-[[4-(2,6-Dimethylphenyl)-6-[(2R)-4,4-dimethyl-2-(spiro[2.3]hexan-5-ylamino)pentoxy]pyrimidin-2-yl]sulfamoyl]benzoic acid

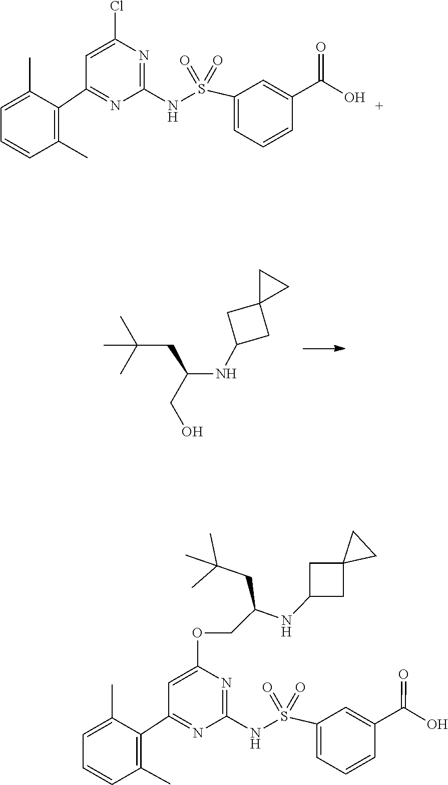 [SMILES: Cc1cccc(C)c1-c1cc(Cl)nc(NS(=O)(=O)c2cccc(C(=O)O)c2)n1 ; CC(C)(C)C[C@H](CO)NC1CC2(CC2)C1 ; Cc1cccc(C)c1-c1cc(OC[C@@H](CC(C)(C)C)NC2CC3(CC3)C2)nc(NS(=O)(=O)c2cccc(C(=O)O)c2)n1]

In a 20 mL vial, to a stirred solution of (2R)-4,4-dimethyl-2-(spiro[2.3]hexan-5-ylamino)pentan-1-ol (145 mg, 0.6861 mmol) in anhydrous tetrahydrofuran (7 mL) was added 3-[[4-chloro-6-(2,6-dimethylphenyl)pyrimidin-2-yl]sulfamoyl]benzoic acid (275 mg, 0.6581 mmol). The heterogeneous mixture was stirred under nitrogen for 2 min to form a milky emulsion. To the emulsion, was added solid potassium tert-butoxide (336 mg, 2.994 mmol) at once. The reaction was stirred for 1.5 h at room temperature. The reaction mixture was partitioned between ethyl acetate (150 mL) and a 1M HCl solution (enough to bring pH to 1). The combined organics were washed with brine (10 mL), dried over anhydrous sodium sulfate, filtered and concentrated under reduced pressure. The crude was purified by reverse-phase HPLC (Cis column) using 1-99% acetonitrile in water (5 mM HCl as modifier) over 15 min to give 3-[[4-(2,6-dimethylphenyl)-6-[(2R)-4,4-dimethyl-2-(spiro[2.3]hexan-5-ylamino)pentoxy]pyrimidin-2-yl]sulfamoyl]benzoic acid (hydrochloride salt) (207 mg, 50%) was obtained as white solid. ESI-MS m/z calc. 592.2719, found 593.4 (M+1)⁺; Retention time: 1.32 minutes; LC method A. ¹H NMR (500 MHz, DMSO-d₆) δ 9.33 (s, 1H), 8.47 (s, 1H), 8.15 (t, J=8.3 Hz, 2H), 7.72 (t, J=7.9 Hz, 1H), 7.27 (t, J=8.4 Hz, 1H), 7.14 (d, J=7.5 Hz, 2H), 6.37 (s, 1H), 4.33 (d, J=12.4 Hz, 1H), 4.21-4.11 (m, 1H), 4.06-3.93 (m, 1H), 3.17 (s, 1H), 2.58-2.52 (m, 2H), 2.19 (t, J=9.8 Hz, 1H), 2.12-2.08 (m, 1H), 2.03 (s, 6H), 1.69 (dd, J=14.8, 8.8 Hz, 1H), 1.60 (d, J=14.4 Hz, 1H), 0.93 (s, 9H), 0.55-0.46 (m, 2H), 0.45-0.37 (m, 2H).

### Step 3: (11R)-6-(2,6-Dimethylphenyl)-11-(2,2-dimethylpropyl)-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (Compound 182)

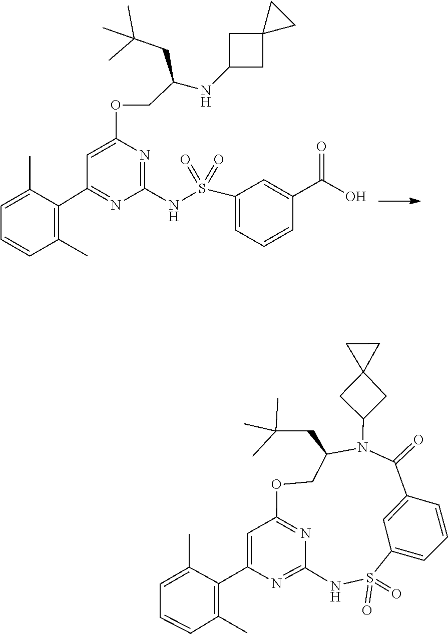 [SMILES: Cc1cccc(C)c1-c1cc(OC[C@@H](CC(C)(C)C)NC2CC3(CC3)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CC3)C1)[C@H](CC(C)(C)C)CO2]

In a 4 mL vial, to a stirred solution of 3-[[4-(2,6-dimethylphenyl)-6-[(2R)-4,4-dimethyl-2-(spiro[2.3]hexan-5-ylamino)pentoxy]pyrimidin-2-yl]sulfamoyl]benzoic acid (hydrochloride salt) (20 mg, 0.03179 mmol) in anhydrous DMF (1.2 mL) were added [dimethylamino(triazolo[4,5-b]pyridin-3-yloxy)methylene]-dimethyl-ammonium (Phosphorus Hexafluoride Ion) (14 mg, 0.03682 mmol) (HATU) and DIEA (30 μL, 0.1722 mmol), in that order. Nitrogen gas was purged for 20 sec and capped. The reaction was stirred at ambient temperature for 6 h. The reaction was diluted with dimethyl sulfoxide (0.5 mL), microfiltered (0.45 uM) and purified from reverse phase preparative HPLC (C₁₈) using a gradient of acetonitrile in water (1 to 99% over 15 min, HCl as a modifier) to give (11R)-6-(2,6-dimethylphenyl)-11-(2,2-dimethylpropyl)-2,2-dioxo-12-spiro[2.3]hexan-5-yl-9-oxa-2λ⁶-thia-3,5,12,19-tetrazatricyclo[12.3.1.14,8]nonadeca-1(18),4(19),5,7,14,16-hexaen-13-one (10 mg, 54%) as a white solid. ESI-MS m/z calc. 574.26135, found 575.4 (M+1)⁺; Retention time: 2.1 minutes (LC method A).

### Example 145: Preparation of Compound 183

### Step 1: 3-[[4-(2,6-Dimethylphenyl)-6-[(2R)-2-(indan-2-ylamino)-4,4-dimethyl-pentoxy]pyrimidin-2-yl]sulfamoyl]benzoic acid

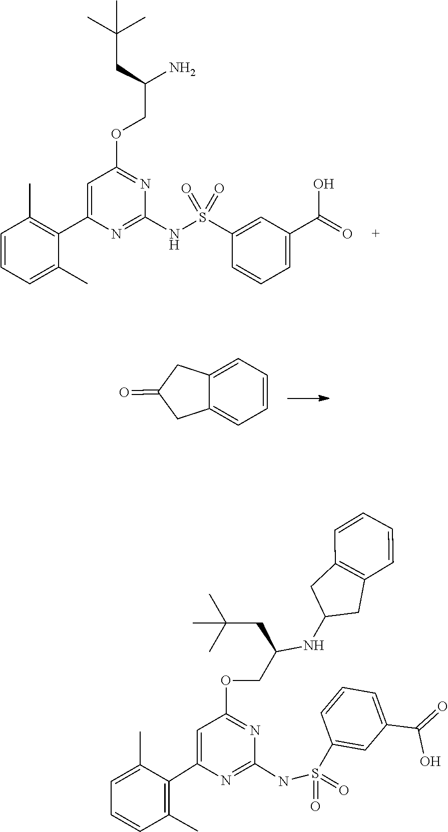

In a 4 mL vial, a stirred mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (40 mg, 0.07285 mmol) and indan-2-one (10 mg, 0.07567 mmol) in anhydrous dichloromethane (0.5 mL) was briefly purged with nitrogen and stirred at ambient temperature for 5 min, Then sodium triacetoxyborohydride (65 mg, 0.3067 mmol) was added and stirring continued for 3 days. Then methanol (0.2 mL) and water (0.1 mL) were added in that order, and the mixture was concentrated under reduced pressure. The residue was taken up in DMSO (1 mL), micro-filtered, and purified from reverse-phase HPLC (1-99% acetonitrile in water over 15 min, HCl as modifier) to give 3-[[4-(2,6-dimethylphenyl)-6-[(2R)-2-(indan-2-ylamino)-4,4-dimethyl-pentoxy]pyrimidin-2-yl]sulfamoyl]benzoic acid (hydrochloride salt) (8 mg, 17%) as white solid. It was used in the subsequent reaction. ESI-MS m/z calc. 628.2719, found 629.3 (M+1)⁺; Retention time: 1.35 minutes; LC method A.

### Step 2: (11R)-6-(2,6-Dimethylphenyl)-11-(2,2-dimethylpropyl)-12-indan-2-yl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 183)

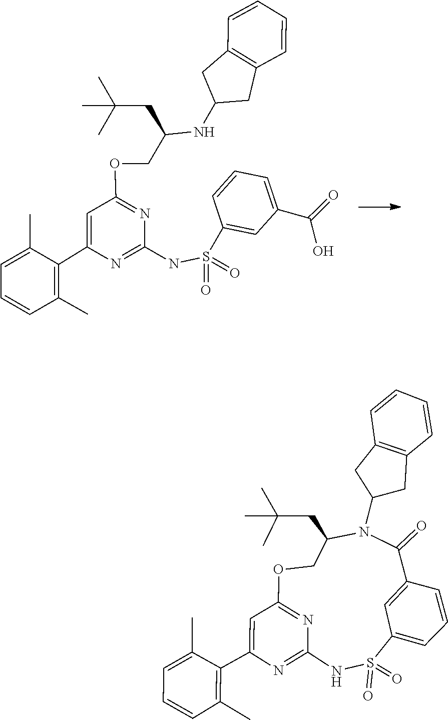

In a 4 mL vial, to a stirred solution of 3-[[4-(2,6-dimethylphenyl)-6-[(2R)-2-(indan-2-ylamino)-4,4-dimethyl-pentoxy]pyrimidin-2-yl]sulfamoyl]benzoic acid (hydrochloride salt) (21 mg, 0.03340 mmol) in anhydrous DMF (1.3 mL) were added 2-chloro-4,6-dimethoxy-1,3,5-triazine (10 mg, 0.05696 mmol) (CDMT) and 4-methylmorpholine (20 μL, 0.1819 mmol), in that order, at ambient temperature under nitrogen. Stirring continued for 24 h, then the solution was micro-filtered, and purified by preparative reverse-phase HPLC (Cis column, 1-99% acetonitrile in water over 15 min, HCl as modifier) to furnish (11R)-6-(2,6-dimethylphenyl)-11-(2,2-dimethylpropyl)-12-indan-2-yl-2,2-dioxo-9-oxa-2λ⁶-thia-3,5,12,19-tetrazatricyclo[12.3.1.14,8]nonadeca-1(18),4(19),5,7,14,16-hexaen-13-one (2.7 mg, 13%) as white solid. ESI-MS m/z calc. 610.26135, found 611.3 (M+1)⁺; Retention time: 2.06 minutes; LC method A.

### Example 146: Preparation of Compound 184

### Step 1: (2R)-2-[(1-tert-Butylpyrazol-4-yl)amino]-4,4-dimethyl-pentan-1-ol

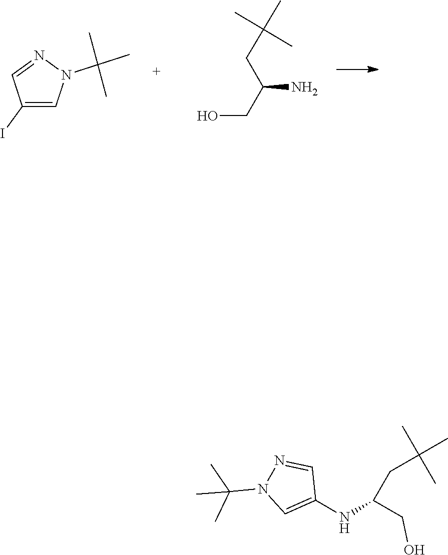

The 1-tert-butyl-4-iodo-pyrazole (approximately 149.2 mg, 0.5965 mmol) was combined with the (2R)-2-amino-4,4-dimethyl-pentan-1-ol (hydrochloride salt) (100 mg, 0.5964 mmol), CuI (approximately 11.36 mg, 0.05965 mmol), and NaOH (approximately 95.43 mg, 2.386 mmol) (ground with mortar and pestle) in a screw cap vial, which was then purged with nitrogen. DMSO (0.3 mL) and water (0.15 mL) were added and the reaction mixture was stirred at 90° C. for 16 hours. After cooling to room temperature, the reaction mixture was diluted with methanol and filtered. The filtrate was concentrated by rotary evaporation and the resulting residue was dissolved in 1:1 DMSO/methanol, filtered a second time and purified by reverse phase HPLC (1-50% ACN in water, HCl modifier, 15 min run) to give the indicated (2R)-2-[(1-tert-butylpyrazol-4-yl)amino]-4,4-dimethyl-pentan-1-ol (hydrochloride salt) (75 mg, 43%) upon drying. ESI-MS m/z calc. 253.21541, found 254.7 (M+1)⁺; Retention time: 0.39 minutes; LC method D.

### Step 2: (11R)-12-(1-tert-Butylpyrazol-4-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 184)

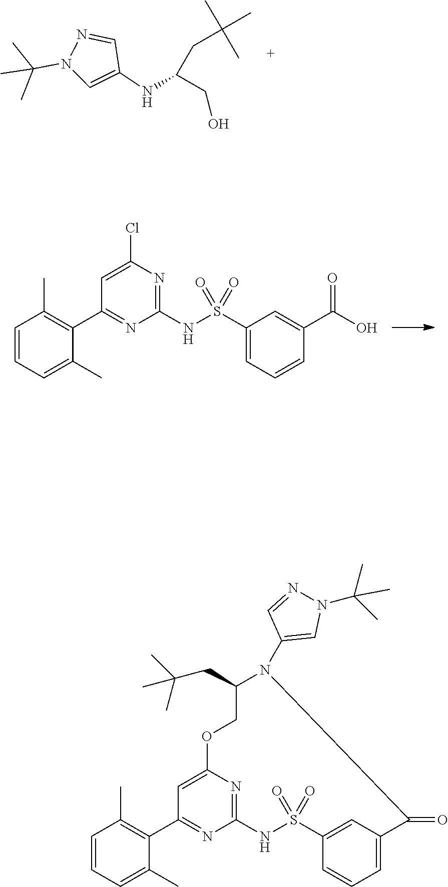 [SMILES: CC(C)(C)C[C@H](CO)Nc1cnn(C(C)(C)C)c1 ; Cc1cccc(C)c1-c1cc(Cl)nc(NS(=O)(=O)c2cccc(C(=O)O)c2)n1 ; Cc1cccc(C)c1-c1cc2nc(n1)NS(=O)(=O)c1cccc(c1)C(=O)N(c1cnn(C(C)(C)C)c1)[C@H](CC(C)(C)C)CO2]

(2R)-2-[(1-tert-Butylpyrazol-4-yl)amino]-4,4-dimethyl-pentan-1-ol (hydrochloride salt) (75 mg, 0.2588 mmol) was combined with 3-[[4-chloro-6-(2,6-dimethylphenyl)pyrimidin-2-yl]sulfamoyl]benzoic acid (approximately 83.20 mg, 0.1991 mmol) in THE (0.75 mL) and stirred until the solids had mostly dissolved/become a suspension. Sodium tert-butoxide (approximately 114.8 mg, 1.195 mmol) was added and the reaction briefly became slightly warm. Stirring was continued for 15 minutes with no external heating. The reaction mixture was then partitioned between 1M HCl and ethyl acetate. The layers were separated, and the aqueous was extracted an additional 4 with ethyl acetate. The combined organics were washed with brine, dried over sodium sulfate, and concentrated. The resulting crude material was dissolved in 1:1 DMSO/methanol, filtered, and purified by reverse phase HPLC (1-60_{%} ACN in water, HCl modifier, 15 min run) to give the SNAr product. The product was dissolved in DMF (8 mL) and NMM (approximately 60.42 mg, 65.67 μL, 0.5973 mmol) was added. The reaction mixture was cooled to 0° C. and CDMT (approximately 52.43 mg, 0.2986 mmol) was added. The reaction was allowed to warm to room temperature as the ice melted and stirred for 48 hours. The reaction mixture was quenched with several drops of water, partially concentrated, diluted with 1:1 DMSO/methanol, filtered, and purified by reverse phase HPLC (1-70% ACN in water, HCl modifier, 30 min run) to give (11R)-12-(1-tert-butylpyrazol-4-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (8.9 mg, 7%). ESI-MS m/z calc. 616.2832, found 617.8 (M+1)⁺; Retention time: 1.87 minutes; LC method A.

### Example 147: Preparation of Compound 185

### Step 1: (2R)-2-[(1-Benzylpyrazol-4-yl)amino]-4,4-dimethyl-pentan-1-ol

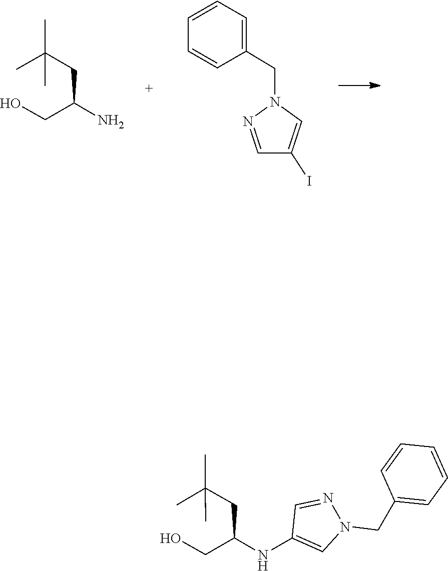

CuI (73 mg, 0.3833 mmol), NaOH (610 mg, 15.251 mmol) (freshly grounded), (2R)-2-amino-4,4-dimethyl-pentan-1-ol (hydrochloride salt) (500 mg, 2.9819 mmol) and 1-benzyl-4-iodo-pyrazole (1.08 g, 3.8015 mmol) were combined in a pressure vial and purged with nitrogen. DMSO (6 mL) and water (3 mL) were added. The reaction mixture was then heated to 90° C. for 16 h. The reaction mixture was then cooled to room temperature, filtered on a Celite pad and rinsed with EtOAc and water. A saturated aqueous solution of ammonium chloride (40 mL) was added to neutralize the base and the product was extracted with EtOAc (3×10 mL). The combined organic layers were washed with 10% brine, dried over magnesium sulfate, filtered and concentrated under reduced pressure. The crude residue was purified by chromatography on a 40 g silica gel cartridge using a gradient of 30-100% EtOAc in heptanes and then 0-20% MeOH in EtOAc to afford (2R)-2-[(1-benzylpyrazol-4-yl)amino]-4,4-dimethyl-pentan-1-ol (300 mg, 35%) as a pale orange oil. ESI-MS m/z calc. 287.19977, found 288.4 (M+1)⁺; Retention time: 1.34 minutes; LC method X. ¹H NMR (400 MHz, CDCl₃) δ 7.37-7.29 (m, 3H), 7.22-7.16 (m, 3H), 6.96 (s, 1H), 5.21 (s, 2H), 3.67 (dd, J=10.8, 3.9 Hz, 1H), 3.34 (dd, J=10.6, 6.7 Hz, 1H), 3.06 (dt, J=10.8, 5.4 Hz, 1H), 2.32 (br. s., 1H), 1.40-1.31 (m, 2H), 0.92 (s, 9H).

### Step 2: 3-[[4-[(2R)-2-[(1-Benzylpyrazol-4-yl)amino]-4,4-dimethyl-pentoxy]-6-(2,6-dimethylphenyl)pyrimidin-2-yl]sulfamoyl]benzoic acid

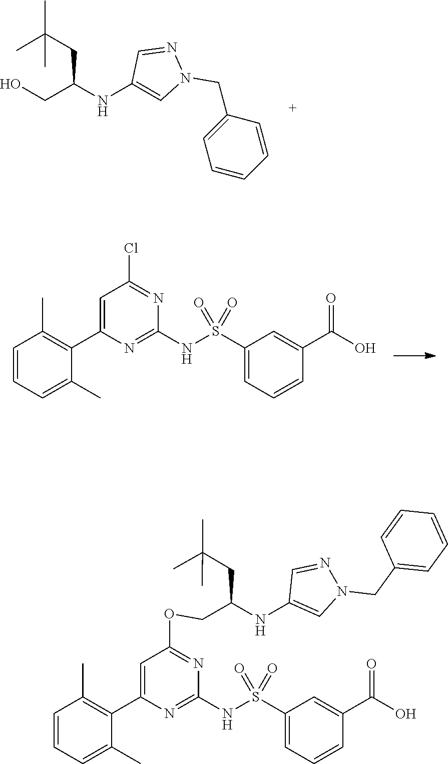 [SMILES: CC(C)(C)C[C@H](CO)Nc1cnn(Cc2ccccc2)c1 ; Cc1cccc(C)c1-c1cc(Cl)nc(NS(=O)(=O)c2cccc(C(=O)O)c2)n1 ; Cc1cccc(C)c1-c1cc(OC[C@@H](CC(C)(C)C)Nc2cnn(Cc3ccccc3)c2)nc(NS(=O)(=O)c2cccc(C(=O)O)c2)n1]

To a solution of (2R)-2-[(1-benzylpyrazol-4-yl)amino]-4,4-dimethyl-pentan-1-ol (900 mg, 3.1315 mmol) in THE (10 mL) was added sodium hydride (60% in mineral oil) (355 mg, 60% w/w, 8.8759 mmol) and the reaction mixture was stirred at room temperature for 0.5 h. Then 3-[[4-chloro-6-(2,6-dimethylphenyl)pyrimidin-2-yl]sulfamoyl]benzoic acid (hydrochloride salt) (1.3 g, 2.8614 mmol) was added and the reaction mixture was stirred at room temperature for 16 h. Another portion of sodium hydride (60% in mineral oil) (115 mg, 60% w/w, 2.8753 mmol) was added and the reaction mixture was stirred at room temperature for 1 h. 2-MeTHF (25 mL) was added followed by 1 N aqueous HCl (25 mL). The phases were separated, and the aqueous phase was washed with 2-MeTHF (2×25 mL). The combined organic layers were washed with brine (25 mL), dried over magnesium sulfate, filtered and concentrated under reduced pressure. The residue was purified twice by reverse phase chromatography on a 50 g C₁₈ cartridge using a gradient of 10-100% MeOH in water (with 0.1% HCl). The pure fractions were lyophilized in MeOH/water to afford 3-[[4-[(2R)-2-[(1-benzylpyrazol-4-yl)amino]-4,4-dimethyl-pentoxy]-6-(2,6-dimethylphenyl)pyrimidin-2-yl]sulfamoyl]benzoic acid (hydrochloride salt) (1.49 g, 74%) as a salmon pink solid. ESI-MS m/z calc. 668.2781, found 669.2 (M+1)⁺; Retention time: 1.83 minutes; LC method X.

### Step 3: (11R)-12-(1-Benzylpyrazol-4-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

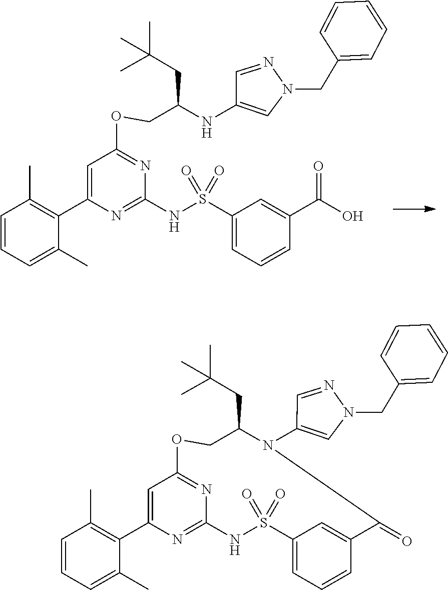 [SMILES: Cc1cccc(C)c1-c1cc(OC[C@@H](CC(C)(C)C)Nc2cnn(Cc3ccccc3)c2)nc(NS(=O)(=O)c2cccc(C(=O)O)c2)n1 ; Cc1cccc(C)c1-c1cc2nc(n1)NS(=O)(=O)c1cccc(c1)C(=O)N(c1cnn(Cc3ccccc3)c1)[C@H](CC(C)(C)C)CO2]

To a 0° C. solution of N-Methylmorpholine (5.8880 g, 6.4 mL, 58.212 mmol) in DMF (1 L) was added 2-chloro-4,6-dimethoxy-1,3,5-triazine (3.5 g, 19.935 mmol) followed by 3-[[4-[(2R)-2-[(1-benzylpyrazol-4-yl)amino]-4,4-dimethyl-pentoxy]-6-(2,6-dimethylphenyl)pyrimidin-2-yl]sulfamoyl]benzoic acid (hydrochloride salt) (9.9 g, 13.448 mmol). After 5 min the reaction went back to room temperature and was stirred for 64 h. The reaction mixture was concentrated under reduced pressure at 50° C. The remaining crude was diluted with DCM (250 mL). That solution was washed with a 1:1 v/v mix of water and brine (4×150 mL), water (2×100 mL) and brine (100 mL). The resulting organic layer was dried over sodium sulfate, filtered and concentrated under reduced pressure. The crude was purified by flash chromatography using a 120 g cartridge, eluting with a gradient of EtOAc in DCM (0 to 100% in 25 CV). Afforded first (10R)-9-(1-benzylpyrazol-4-yl)-6-(2,6-dimethylphenyl)-10-(2,2-dimethylpropyl)-2,2-dioxo-12-oxa-2λ⁶-thia-3,5,9,19-tetrazatricyclo[12.3.1.14,8]nonadeca-1(18),4(19),5,7,14,16-hexaen-13-one (750 mg, 8%) as an off-white solid ESI-MS m/z calc. 650.2675, found 651.2 (M+1)⁺; Retention time: 4.27 minutes and then (11R)-12-(1-benzylpyrazol-4-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (1.4 g, 14%) as an off-white solid ESI-MS m/z calc. 650.2675, found 651.2 (M+1)⁺; Retention time: 4.27 minutes; LC method Y.

### Step 4: (11R)-6-(2,6-Dimethylphenyl)-11-(2,2-dimethylpropyl)-2,2-dioxo-12-(1H-pyrazol-4-yl)-9-oxa-2λ⁶-thia-3,5,12,19-tetrazatricyclo[12.3.1.14,8]nonadeca-1(18),4(19),5,7,14,16-hexaen-13-one (Compound 185)

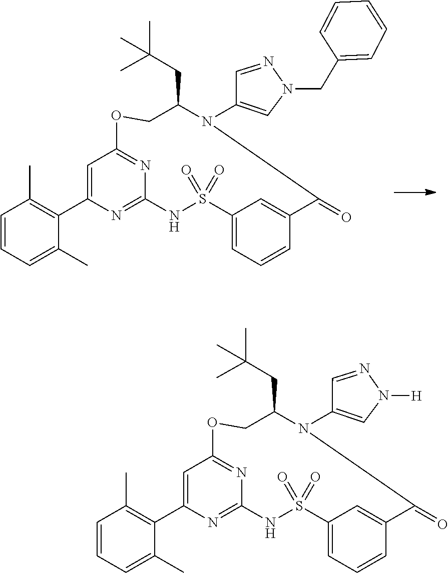 [SMILES: Cc1cccc(C)c1-c1cc2nc(n1)NS(=O)(=O)c1cccc(c1)C(=O)N(c1cnn(Cc3ccccc3)c1)[C@H](CC(C)(C)C)CO2 ; Cc1cccc(C)c1-c1cc2nc(n1)NS(=O)(=O)c1cccc(c1)C(=O)N(c1cn[nH]c1)[C@H](CC(C)(C)C)CO2]

To palladium hydroxide 20% w/w, 50% water (280 mg, 10% w/w, 0.1994 mmol) in a sealed tube was added a solution of (11R)-12-(1-benzylpyrazol-4-yl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (1.4 g, 2.1491 mmol) in MeOH (10 mL). The tube was pressurized at 70 PSI of hydrogen and the reaction was warmed to 70° C. for 24 h. The reaction mixture went back to room temperature and the tube was purged with N2. The crude was filtered over diatomaceous earth, rinsing with MeOH (80 mL). Volatiles were removed under vacuo. Crude material was dissolved again in MeOH (10 mL) and added to palladium hydroxide 20% w/w, 50% water (320 mg, 10% w/w, 0.2279 mmol). The tube was pressurized at 70 PSI of hydrogen and the reaction was warmed to 70° C. for 24 h. The reaction mixture went back to room temperature and the tube was purged with N2. The crude was filtered over diatomaceous earth, rinsing with MeOH (70 mL) and MeTHF (40 mL). Volatiles were removed under vacuo. Crude material was dissolved again in MeOH (10 mL) and added to palladium hydroxide 20% w/w, 50% water (260 mg, 10% w/w, 0.1851 mmol). The tube was pressurized at 70 PSI of hydrogen and the reaction was warmed to 70° C. for 24 h. The reaction mixture went back to room temperature and the tube was purged with N2. The crude was filtered over diatomaceous earth, rinsing with a 1:1 v/v mix of MeOH and MeTHF (200 mL). Volatiles were removed under vacuo. The crude residue was purified by reverse phase chromatography on two equal batches using each time a 20 g C₁₈ cartridge, eluting with a gradient of MeCN in acidic water (0.1% v/v formic acid) of 40 to 70% in 15 CV. Fractions containing the targeted compound were concentrated under vacuo to a third of the volume and the resulting solid was filtered. Afforded (11R)-6-(2,6-dimethylphenyl)-11-(2,2-dimethylpropyl)-2,2-dioxo-12-(1H-pyrazol-4-yl)-9-oxa-2λ⁶-thia-3,5,12,19-tetrazatricyclo[12.3.1.14,8]nonadeca-1(18),4(19),5,7,14,16-hexaen-13-one (320 mg, 23%) as a white solid. ESI-MS m/z calc. 560.2206, found 561.2 (M+1)⁺; Retention time: 3.54 minutes; LC method Y. ¹H NMR (400 MHz, DMSO-d₆) δ 13.05 (br. s., 1H), 8.72 (br. s., 1H), 7.97 (br. s., 2H), 7.73 (br. s., 2H), 7.69-7.58 (m, 1H), 7.26 (t, J=7.8 Hz, 1H), 7.17-7.07 (m, 2H), 6.40 (br. s., 1H), 5.44 (d, J=7.6 Hz, 1H), 4.04-3.89 (m, 2H), 2.26-1.82 (m, 6H), 1.53 (dd, J=15.0, 7.2 Hz, 1H), 1.17 (d, J=14.9 Hz, 1H), 0.55 (s, 9H).

### Example 148: Preparation of Compound 186

### Step 1: 3-[[4-chloro-6-[(2R)-4-methyl-2-(spiro[2.3]hexan-5-ylamino)pentoxy]pyrimidin-2-yl]sulfamoyl]benzoic acid

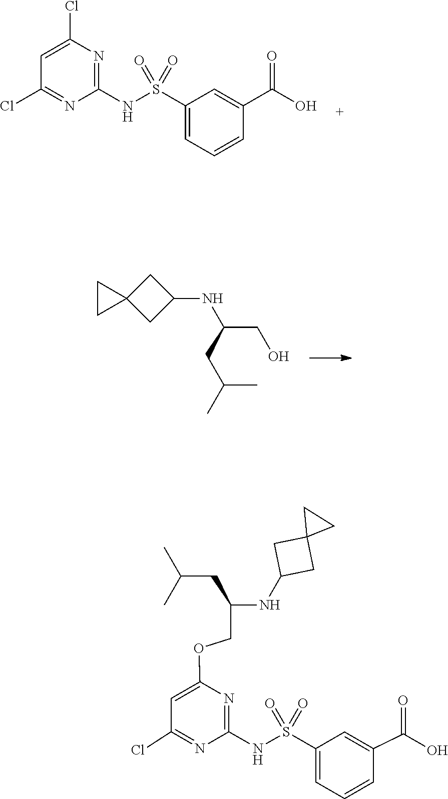 [SMILES: O=C(O)c1cccc(S(=O)(=O)Nc2nc(Cl)cc(Cl)n2)c1 ; CC(C)C[C@H](CO)NC1CC2(CC2)C1 ; CC(C)C[C@H](COc1cc(Cl)nc(NS(=O)(=O)c2cccc(C(=O)O)c2)n1)NC1CC2(CC2)C1]

In a reaction vial, 3-[(4,6-dichloropyrimidin-2-yl)sulfamoyl]benzoic acid (2.02 g, 5.802 mmol) and (2R)-4-methyl-2-(spiro[2.3]hexan-5-ylamino)pentan-1-ol (hydrochloride salt) (1.37 g, 5.860 mmol) were dissolved in THF (12.5 mL) then sodium tert-butoxide (2.79 g, 29.03 mmol) was added. The reaction was stirred for 45 min. at rt The reaction mixture was then partitioned between ethyl acetate and a 1M HCl solution. The organics were separated, washed with brine, dried over sodium sulfate, and evaporated to dryness. The solid material was slurried in 50% ethyl acetate/hexanes then filtered to provide the product (HCl salt). 3-[[4-chloro-6-[(2R)-4-methyl-2-(spiro[2.3]hexan-5-ylamino)pentoxy]pyrimidin-2-yl]sulfamoyl]benzoic acid (hydrochloride salt) (2.6157 g, 80%). ESI-MS m/z calc. 508.15472, found 509.3 (M+1)⁺; Retention time: 1.24 minutes; (LC method A).

### Step 2: (11R)-6-Chloro-11-isobutyl-2,2-dioxo-12-spiro[2.3]hexan-5-yl-9-oxa-2%⁶-thia-3,5,12,19-tetrazatricyclo[12.3.1.14,8]nonadeca-1(18),4,6,8(19),14,16-hexaen-13-one

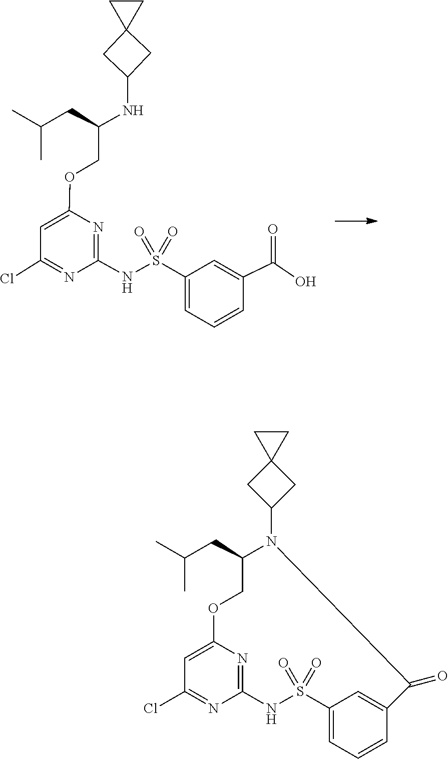 [SMILES: CC(C)C[C@H](COc1cc(Cl)nc(NS(=O)(=O)c2cccc(C(=O)O)c2)n1)NC1CC2(CC2)C1 ; CC(C)C[C@@H]1COc2cc(Cl)nc(n2)NS(=O)(=O)c2cccc(c2)C(=O)N1C1CC2(CC2)C1]

HATU (3.8 g, 9.9940 mmol) was added to a solution of 3-[[4-chloro-6-[(2R)-4-methyl-2-(spiro[2.3]hexan-5-ylamino)pentoxy]pyrimidin-2-yl]sulfamoyl]benzoic acid (hydrochloride salt) (2.7 g, 4.9498 mmol) and DIEA (3.2 g, 4.3127 mL, 24.760 mmol) in DMF (300 mL), and the resulting mixture was allowed to stir for seventeen hours at room temperature. The reaction mixture was added dropwise to rapidly stirring water (1.5 L), then the pH was adjusted to 3-4 using 1M hydrochloric acid. The resulting precipitate was collected by vacuum filtration, then was triturated with water (50 mL). The solid was collected by vacuum filtration, dried in vacuo, then purified by silica gel chromatography using 0 to 60% ethyl acetate in hexane to obtain (11R)-6-chloro-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4,6,8(19),14,16-hexaen-13-one (1.4 g, 58%). ESI-MS m/z calc. 490.14417, found 491.7 (M+1)⁺; Retention time: 6.64 minutes; LC method S.

### Step 3: (11R)-6-Chloro-11-isobutyl-3-(methoxymethyl)-2,2-dioxo-12-spiro[2.3]hexan-5-yl-9-oxa-2λ⁶-thia-3,5,12,19-tetrazatricyclo[12.3.1.14,8]nonadeca-1(18),4,6,8(19),14,16-hexaen-13-one

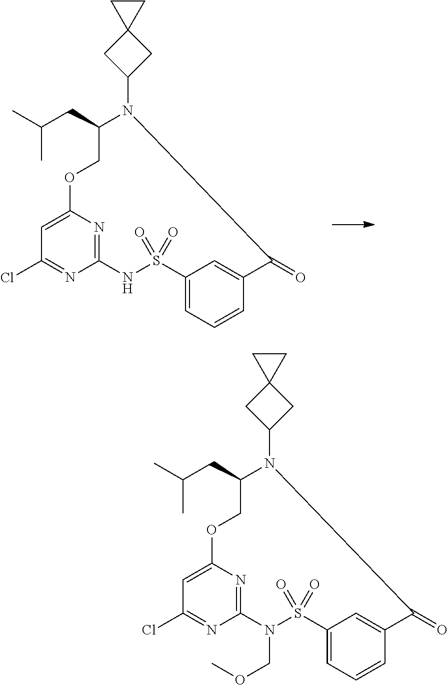

Potassium carbonate (236 mg, 1.7076 mmol) was added to a solution of (11R)-6-chloro-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4,6,8(19),14,16-hexaen-13-one (300 mg, 0.6110 mmol) and chloro(methoxy)methane (92 mg, 0.0868 mL, 1.1427 mmol), and the resulting slurry was allowed to stir at room temperature for four hours. The reaction was quenched with a small amount of water, then was diluted with DCM (6 mL). The phases were separated: the aqueous phase was discarded and the organic phase was dried in vacuo and purified by silica gel chromatography using 0-60% diethyl ether in hexane to obtain (11R)-6-chloro-11-isobutyl-3-(methoxymethyl)-2,2-dioxo-12-spiro[2.3]hexan-5-yl-9-oxa-2λ⁶-thia-3,5,12,19-tetrazatricyclo[12.3.1.14,8]nonadeca-1(18),4,6,8(19),14,16-hexaen-13-one (190 mg, 55%). ESI-MS m/z calc. 534.17035, found 535.2 (M+1)⁺; Retention time: 7.44 minutes; LC method S.

### Step 4: (11R)-6-[2,6-Bis(trifluoromethyl)phenyl]-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4,6,8(19),14,16-hexaen-13-one (Compound 186)

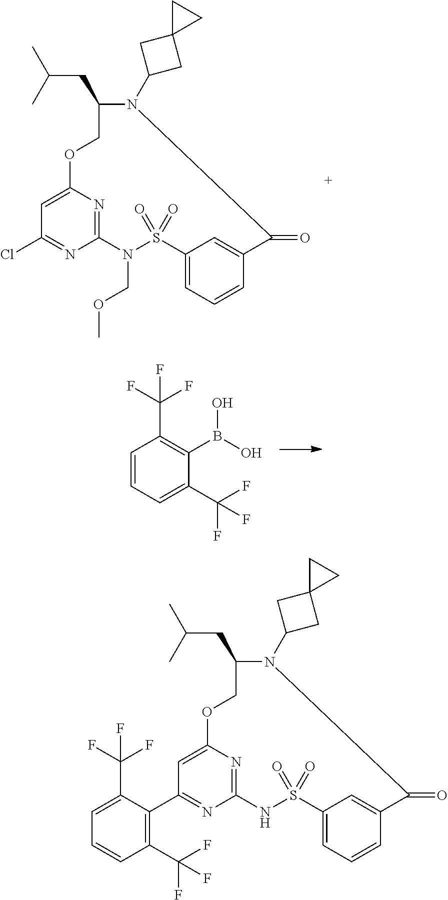 [SMILES: COCN1c2nc(Cl)cc(n2)OC[C@@H](CC(C)C)N(C2CC3(CC3)C2)C(=O)c2cccc(c2)S1(=O)=O ; OB(O)c1c(C(F)(F)F)cccc1C(F)(F)F ; CC(C)C[C@@H]1COc2cc(-c3c(C(F)(F)F)cccc3C(F)(F)F)nc(n2)NS(=O)(=O)c2cccc(c2)C(=O)N1C1CC2(CC2)C1]

In a reaction vial, (11R)-6-chloro-11-isobutyl-3-(methoxymethyl)-2,2-dioxo-12-spiro[2.3]hexan-5-yl-9-oxa-2λ⁶-thia-3,5,12,19-tetrazatricyclo[12.3.1.14,8]nonadeca-1(18),4,6,8(19),14,16-hexaen-13-one (97.7 mg, 0.1826 mmol) and [2,6-bis(trifluoromethyl)phenyl]boronic acid (94.1 mg, 0.3648 mmol) was mixed with cesium carbonate (178.3 mg, 0.5472 mmol) in 0.990 mL of (10:1) Dioxane (900 μL)/water (90 μL). The reaction mixture was purged with nitrogen and Pd(dppf)Cl₂ (59.6 mg, 0.07298 mmol) was added. The reaction was heated in at 100° C. for 12 h. then evaporated to dryness. The reaction was partitioned between ethyl acetate and saturated NaCl solution. The organic layer was isolated, dried over anhydrous sodium sulfate, filtered, and evaporated to dryness. The residue was dissolved in (1:1) TFA (1.48 g, 12.98 mmol)/DCM (1 mL) and stirred at rt for 30 min. The reaction was evaporated to dryness and purified by preparative HPLC using 0-100% water/ACN gradient with HCl modifier to give (11R)-6-[2,6-bis(trifluoromethyl)phenyl]-11-isobutyl-2,2-dioxo-12-spiro[2.3]hexan-5-yl-9-oxa-2λ⁶-thia-3,5,12,19-tetrazatricyclo[12.3.1.14,8]nonadeca-1(18),4,6,8(19),14,16-hexaen-13-one (3.4 mg, 3%). ESI-MS m/z calc. 668.1892, found 669.4 (M+1)⁺; Retention time: 1.71 minutes; LC method A.

### Example 149: Preparation of Compound 187

### Step 1: 3-[[4-[(2R)-2-[(7-tert-Butoxycarbonyl-7-azaspiro[3.5]nonan-2-yl)amino]-5,5,5-trifluoro-4,4-dimethyl-pentoxy]-6-(2,6-dimethylphenyl)pyrimidin-2-yl]sulfamoyl]benzoic acid

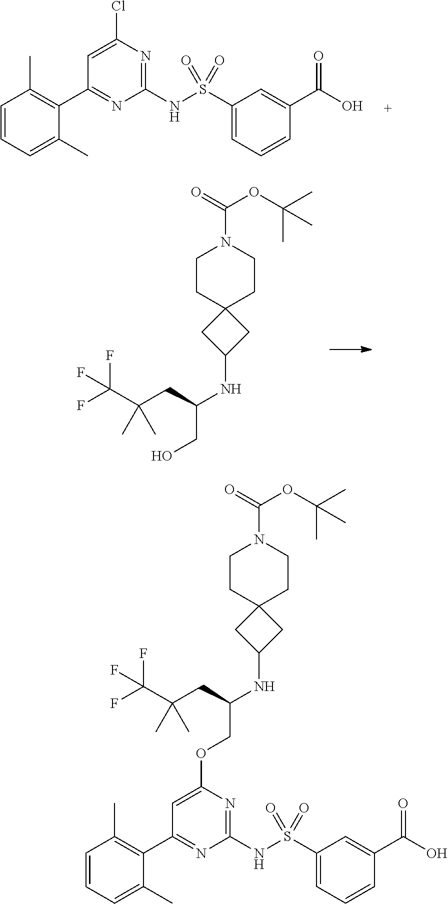

In a 20 mL vial, 3-[[4-chloro-6-(2,6-dimethylphenyl)pyrimidin-2-yl]sulfamoyl]benzoic acid (1.334 g, 3.192 mmol) and tert-butyl 2-[[(1R)-4,4,4-trifluoro-1-(hydroxymethyl)-3,3-dimethyl-butyl]amino]-7-azaspiro[3.5]nonane-7-carboxylate (1.314 g, 3.217 mmol) were charged under nitrogen with anhydrous THE (11 mL). Sodium tert-butoxide (1.48 g, 15.40 mmol) was added (slight exotherm). The reaction was stirred at room temperature for 4 hours. More sodium tert-butoxide (546 mg, 5.681 mmol) was added and the mixture was stirred for an additional 2 hours. The mixture was partitioned between ethyl acetate (50 mL) and aqueous 1M HCl (40 mL) and brine (20 mL). After separation, the aqueous phase was further extracted with EtOAc (2×30 mL). The combined extracts were dried over sodium sulfate and the solvents evaporated to give a solid. The solid was triturated in EtOAc/hexanes and filtered. After drying in vacuo, 3-[[4-[(2R)-2-[(7-tert-butoxycarbonyl-7-azaspiro[3.5]nonan-2-yl)amino]-5,5,5-trifluoro-4,4-dimethyl-pentoxy]-6-(2,6-dimethylphenyl)pyrimidin-2-yl]sulfamoyl]benzoic acid (hydrochloride salt) (1.867 g, 71%) was isolated as a tan solid. ESI-MS m/z calc. 789.3383, found 790.5 (M+1)⁺; Retention time: 1.55 minutes. (LC method A).

### Step 2: tert-Butyl 2-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]-7-azaspiro[3.5]nonane-7-carboxylate (Compound 187)

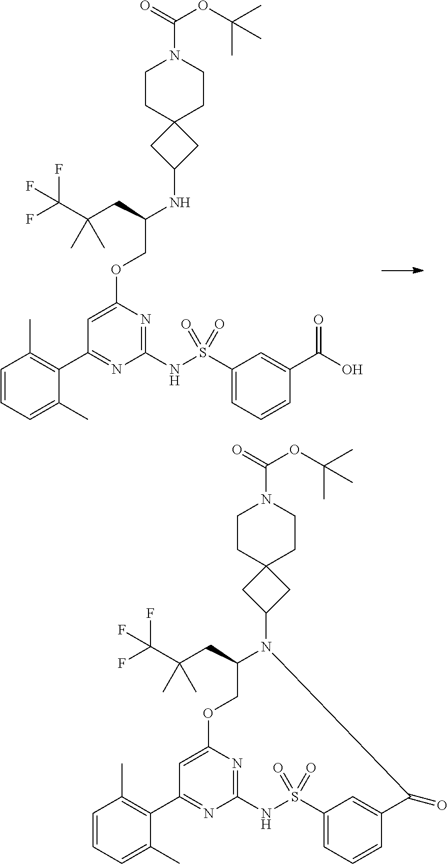

A 250 mL flask was charged under nitrogen with HATU (1.75 g, 4.602 mmol), anhydrous DMF (40 mL) and DIEA (2 mL, 11.48 mmol). A solution of 3-[[4-[(2R)-2-[(7-tert-butoxycarbonyl-7-azaspiro[3.5]nonan-2-yl)amino]-5,5,5-trifluoro-4,4-dimethyl-pentoxy]-6-(2,6-dimethylphenyl)pyrimidin-2-yl]sulfamoyl]benzoic acid (hydrochloride salt) (1.867 g, 2.259 mmol) in anhydrous DMF (20 mL) was added dropwise through syringe over a period of 10 minutes. The mixture was stirred at room temperature for 17 hours. The mixture was concentrated. The residue was partitioned between EtOAc (50 mL) and 1N HCl (1 M). The aqueous phase was washed with brine (25 mL). After drying over sodium sulfate and evaporation of the solvents, the solid was dissolved in DCM and a bit of methanol and purified by flash chromatography on silica gel (120 g column) using a gradient of ethyl acetate (15 to 100% over 30 min) in hexanes. The product eluted around 60% EtOAc. The pure fractions were combined and the solvents evaporated to give tert-butyl 2-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]-7-azaspiro[3.5]nonane-7-carboxylate (261 mg, 15%) as a white solid. ESI-MS m/z calc. 771.32776, found 772.46 (M+1)⁺; Retention time: 2.18 minutes (LC method A).

### Example 150: Preparation of Compound 188, Compound 189, and Compound 190

### Step 1: (11R)-12-{7-azaspiro[3.5]nonan-2-yl}-6-(2,6-dimethylphenyl)-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione

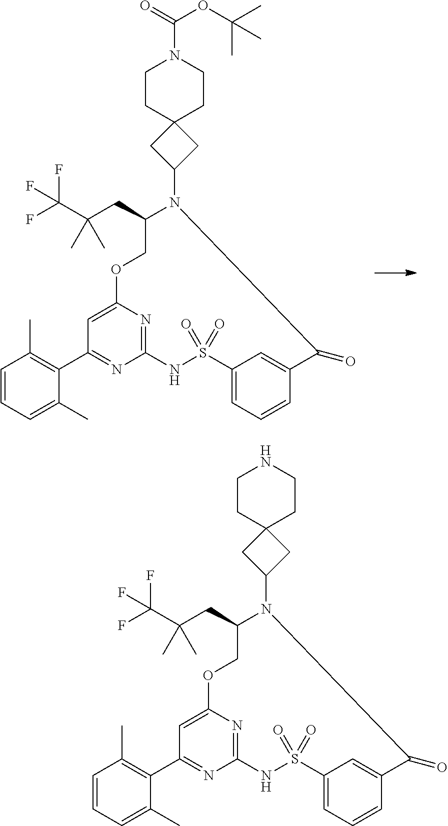

A 100 mL flask containing tert-butyl 2-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]-7-azaspiro[3.5]nonane-7-carboxylate (257 mg, 0.3263 mmol) was treated with DCM (2 mL) and HCl (1 mL of 4 M, 4.000 mmol) (4M in dioxane) at room temperature for 2 hours. The volatiles were removed by evaporation. DCM and hexanes were added, and the solvents were evaporated. The operation was repeated until a white solid was obtained. Drying under vacuum gave (11R)-12-{7-azaspiro[3.5]nonan-2-yl}-6-(2,6-dimethylphenyl)-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (hydrochloride salt) (256 mg, 111%) as a white solid. ESI-MS m/z calc. 671.2753, found 672.35 (M+1)⁺; Retention time: 1.43 minutes (LC method A).

### Step 2: (11R)-6-(2,6-dimethylphenyl)-12-[7-(2-methoxyethyl)-7-azaspiro[3.5]nonan-2-yl]-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (Compound 190), and (11R)-6-(2,6-dimethylphenyl)-12-[7-(3-methoxypropyl)-7-azaspiro[3.5]nonan-2-yl]-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (Compound 188), and (11R)-6-(2,6-dimethylphenyl)-12-{7-[2-(propan-2-yloxy)ethyl]-7-azaspiro[3.5]nonan-2-yl}-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (Compound 189)

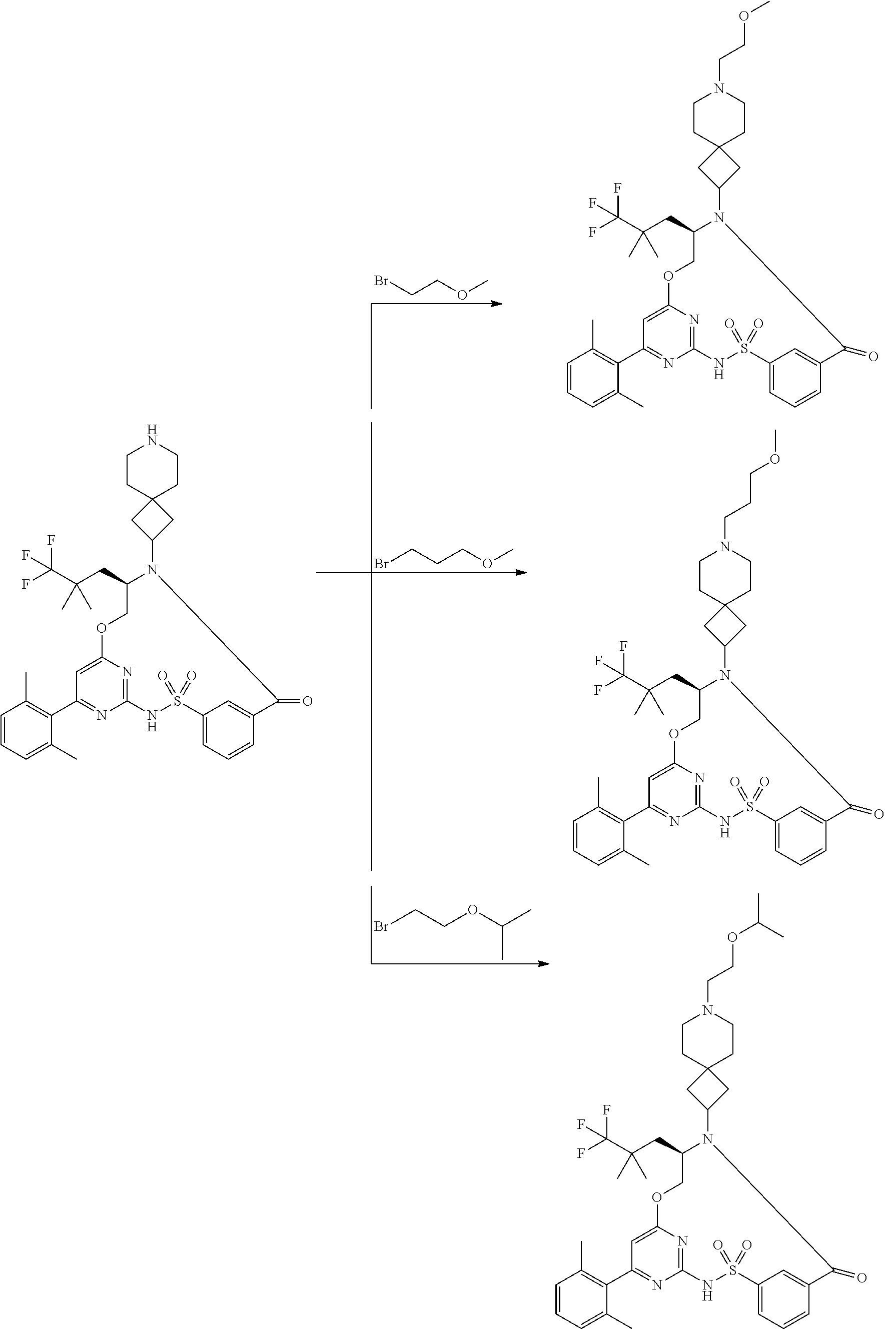

Three alkylation reactions were run in separate vials. For each reaction, a 4 mL vial was charged with under nitrogen with crude (11R)-12-{7-azaspiro[3.5]nonan-2-yl}-6-(2,6-dimethylphenyl)-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (hydrochloride salt) (25 mg, 0.03530 mmol), anhydrous acetonitrile (500 μL), DIEA (35 μL, 0.2009 mmol) and 1-bromo-2-methoxy-ethane (11 μL, 0.1171 mmol) (reaction A), 1-bromo-3-methoxy-propane (12 μL, 0.1067 mmol) (reaction B) or 2-(2-bromoethoxy)propane (18 mg, 0.1078 mmol) (reaction C). The vial was briefly purged with nitrogen, capped and vigorously stirred at 55° C. for 15 hours. (reaction B complete). For reaction A and C, more alkylating agent was added (same amount as before) and the mixtures were stirred for another 7 hours (reaction C complete). To the reaction A was added another amount of alkylating agent and 2 drops of DMSO and it was stirred overnight. For each reaction, the solution was diluted in DMSO (0.5 mL), microfiltered through a syringe filter disc and purified by reverse phase preparative HPLC (C₁₈) using a gradient of acetonitrile in water (1 to 99% over 15 min) and HCl as a modifier give separately the three compounds. Reaction A: (11R)-6-(2,6-dimethylphenyl)-12-[7-(2-methoxyethyl)-7-azaspiro[3.5]nonan-2-yl]-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (hydrochloride salt) (6 mg, 22%). ESI-MS m/z calc. 729.3172, found 730.77 (M+1)⁺; Retention time: 1.38 minutes (LC method A); Reaction B: (11R)-6-(2,6-dimethylphenyl)-12-[7-(3-methoxypropyl)-7-azaspiro[3.5]nonan-2-yl]-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (hydrochloride salt) (9.7 mg, 34%). ESI-MS m/z calc. 743.3328, found 744.78 (M+1)⁺; Retention time: 1.39 minutes (LC method A); and Reaction C: (11R)-6-(2,6-dimethylphenyl)-12-{7-[2-(propan-2-yloxy)ethyl]-7-azaspiro[3.5]nonan-2-yl}-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (hydrochloride salt) (12.3 mg, 44%). ESI-MS m/z calc. 757.34845, found 758.97 (M+1)⁺; Retention time: 1.49 minutes (LC method A).

### Example 151: Preparation of Compound 191

### Step 1: Methyl 2-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]-7-azaspiro[3.5]nonane-7-carboxylate (Compound 191)

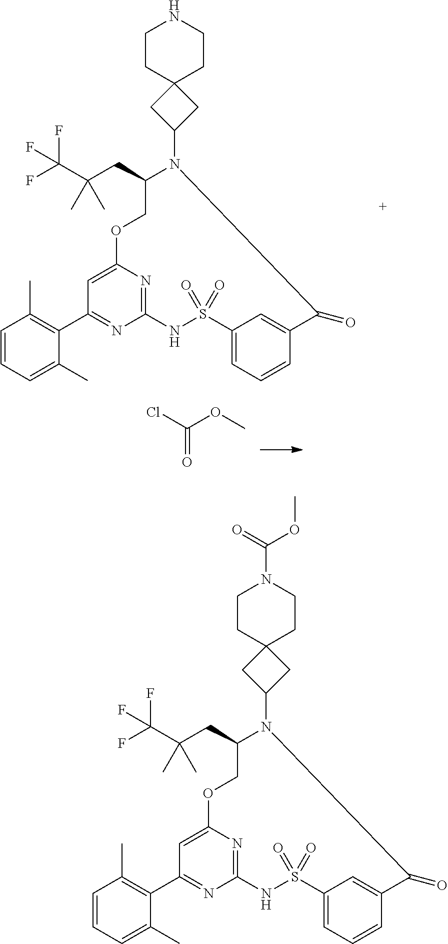

A 4 mL vial was charged with (11R)-12-{7-azaspiro[3.5]nonan-2-yl}-6-(2,6-dimethylphenyl)-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (hydrochloride salt) (22 mg, 0.03106 mmol), DCM (0.5 mL) and DIEA (28 μL, 0.1608 mmol). methyl chloroformate (5 μL, 0.06471 mmol) (neat) was added and the mixture was stirred at room temperature for 1 hour. A bit of methanol was added, and the solvents were evaporated. DMSO (1 mL) was added. The solution was microfiltered through a syringe filter disc and purified by reverse phase preparative HPLC (C₁₈) using a gradient of acetonitrile in water (1 to 99% over 15 min) and HCl as a modifier to give methyl 2-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]-7-azaspiro[3.5]nonane-7-carboxylate (12 mg, 53%) as a white solid. ESI-MS m/z calc. 729.28076, found 730.7 (M+1)⁺; Retention time: 1.69 minutes (LC method A).

### Example 152: Preparation of Compound 192, Compound 193, and Compound 194

### Step 1: 3-[[4-[(2R)-2-[[3-(tert-Butoxycarbonylamino)cyclobutyl]amino]-5,5,5-trifluoro-4,4-dimethyl-pentoxy]-6-(2,6-dimethylphenyl)pyrimidin-2-yl]sulfamoyl]benzoic acid

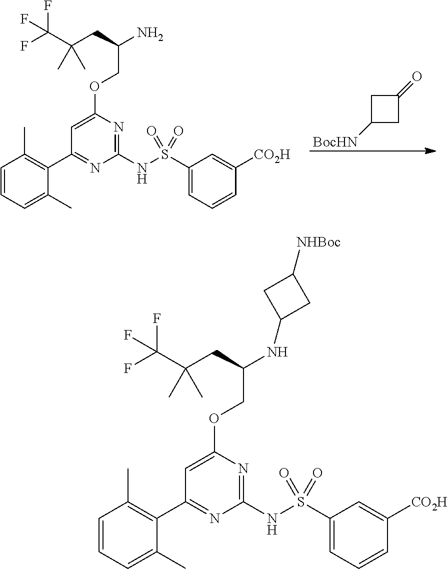 [SMILES: Cc1cccc(C)c1-c1cc(OC[C@H](N)CC(C)(C)C(F)(F)F)nc(NS(=O)(=O)c2cccc(C(=O)O)c2)n1 ; CC(C)(C)OC(=O)NC1CC(=O)C1 ; Cc1cccc(C)c1-c1cc(OC[C@@H](CC(C)(C)C(F)(F)F)NC2CC(NC(=O)OC(C)(C)C)C2)nc(NS(=O)(=O)c2cccc(C(=O)O)c2)n1]

A 100 mL flask was charged under nitrogen with 3-[[4-[(2R)-2-amino-5,5,5-trifluoro-4,4-dimethyl-pentoxy]-6-(2,6-dimethylphenyl)pyrimidin-2-yl]sulfamoyl]benzoic acid (hydrochloride salt) (2.08 g, 3.208 mmol), tert-butyl N-(3-oxocyclobutyl)carbamate (597 mg, 3.223 mmol), anhydrous DCM (5 mL) j and the mixture was stirred for 5-10 minutes until all solids dissolved. Sodium acetoxyborohydride (Sodium salt) (2.51 g, 11.84 mmol) was added and the mixture was stirred at rt for 2 hours. The mixture was put in a freezer overnight. More tert-butyl N-(3-oxocyclobutyl)carbamate (342 mg, 1.846 mmol), and DCM (2 mL) and sodium triacetoxyborohydride (Sodium salt) (730 mg, 3.444 mmol) were added and the mixture was stirred at rt for 4 hours (90% conversion). More sodium triacetoxyborohydride (730 mg, 3.444 mmol) was added and the mixture was stirred for an additional 3 hours (no further evolution). The reaction was quenched by the slow addition of MeOH (10 mL), 1N HCl (30 mL) and ethyl acetate (30 mL). Brine was added and the two phases were separated. The organic phase was extracted with EtOAc (30 mL). The combined extracts were dried over sodium sulfate and concentrated. The residue was triturated in diethylether (150 mL) and the resulting off-white solid was filtered and dried to give crude 3-[[4-[(2R)-2-[[3-(tert-butoxycarbonylamino)cyclobutyl]amino]-5,5,5-trifluoro-4,4-dimethyl-pentoxy]-6-(2,6-dimethylphenyl)pyrimidin-2-yl]sulfamoyl]benzoic acid (hydrochloride salt) (2.111 g, 85%) as a mixture of isomer (approximative ratio 70:30). ESI-MS m/z calc. 735.2914, found 736.63 (M+1)⁺; Retention time: 1.35 minutes (major isomer) and retention time: 1.36 minutes (minor isomer) (LC method A).

### Step 2: tert-Butyl N-{3-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]cyclobutyl}carbamate, major diastereomer 1 (Compound 193), and tert-butyl N-{3-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]cyclobutyl}carbamate, minor diastereomer 2 (Compound 194)

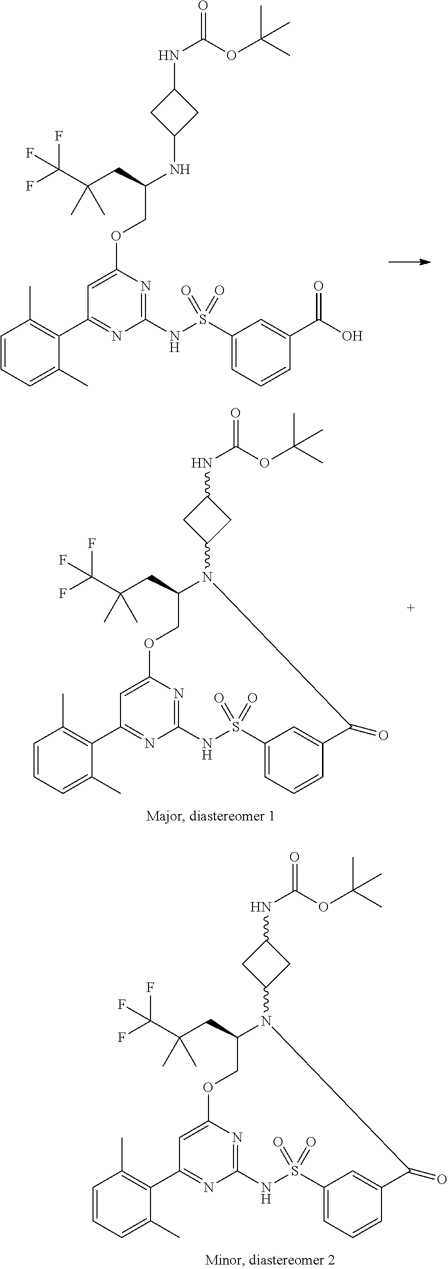

A 250 mL flask was charged under nitrogen with COMU (2.398 g, 5.599 mmol), anhydrous DMF (60 mL) and DIEA (2.5 mL, 14.35 mmol). A solution of 3-[[4-[(2R)-2-[[3-(tert-butoxycarbonylamino)cyclobutyl]amino]-5,5,5-trifluoro-4,4-dimethyl-pentoxy]-6-(2,6-dimethylphenyl)pyrimidin-2-yl]sulfamoyl]benzoic acid (hydrochloride salt) (2.111 g, 2.733 mmol) in anhydrous DMF (40 mL) was added dropwise through syringe over a period of 5 minutes. The mixture was stirred at room temperature for 22 hours. The mixture was concentrated and diluted with DCM. The crude solution was purified by flash chromatography on silica gel (120 g column) using a gradient of ethyl acetate (10 to 100% over 30 min) in hexanes. The product eluted around 60-70% EA (as two non-baseline separated peaks). Evaporation of the solvents gave crude tert-butyl N-{3-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]cyclobutyl}carbamate (852 mg, 43%) as a yellow solid and a mixture of isomers (70:30 ratio). ESI-MS m/z calc. 717.28076, found 718.68 (M+1)⁺; Retention time: 1.91 minutes (major) and Retention time: 1.95 minutes (minor) (LC method A). 163 mg of this crude mixture was taken aside

The rest of the material (698 mg) was dissolved in DMSO (8 mL). The solution was microfiltered through syringe filter disc and purified by reverse phase preparative HPLC (C₁₈) using a gradient of acetonitrile in water (40 to 80% over 20 min) and HCl as a modifier to provide two isomers. For each isomer, the pure fractions were combined. The organic solvent was evaporated. A bit of brine was added, and the product was extracted with EtOAc. After drying over sodium sulfate, the residue was triturated in EtOAc/hexanes and the solvents evaporated to give the products. Major and more polar diastereomer 1, tert-butyl N-{3-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]cyclobutyl}carbamate (335 mg, 30%). ESI-MS m/z calc. 717.28076, found 718.75 (M+1)⁺; Retention time: 1.92 minutes (LC method A), ¹H NMR (400 MHz, DMSO-d₆) δ 13.56-11.81 (broad m, 1H), 8.44 (s, 1H), 7.93 (s, 1H), 7.69 (s, 2H), 7.34-7.19 (m, 2H), 7.12 (d, J=7.6 Hz, 2H), 6.42 (br s, 1H), 5.14 (dd, J=11.0, 4.4 Hz, 1H), 4.39-4.15 (m, 2H), 4.07 (br s, 1H), 3.82 (br s, 1H), 3.18 (dt, J=28.6, 9.2 Hz, 2H), 2.26-1.67 (m, 10H), 1.40 (s, 9H), 0.84 (s, 3H), 0.59 (s, 3H); and a minor and less polar diastereomer 2, tert-butyl N-{3-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]cyclobutyl}carbamate (152 mg, 31%). ESI-MS m/z calc. 717.28076, found 718.71 (M+1)⁺; Retention time: 1.97 minutes (LC method A). ¹H NMR (400 MHz, DMSO-d₆) δ 13.59-11.95 (broad m, 1H), 8.45 (s, 1H), 7.93 (s, 1H), 7.68 (s, 2H), 7.35-7.18 (m, 1H), 7.18-7.00 (m, 3H), 6.39 (s, 1H), 5.16 (dd, J=11.1, 4.3 Hz, 1H), 4.34 (t, J=11.2 Hz, 1H), 3.88-3.48 (m, 3H), 2.87-2.70 (m, 2H), 2.62-2.52 (m, 2H, overlapped with DMSO), 2.27-1.81 (m, 7H), 1.75 (d, J=15.7 Hz, 1H), 1.39 (s, 9H), 0.86 (s, 3H), 0.64 (s, 3H).

### Step 3: (11R)-12-(3-aminocyclobutyl)-6-(2,6-dimethylphenyl)-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione, major diasteromer 1

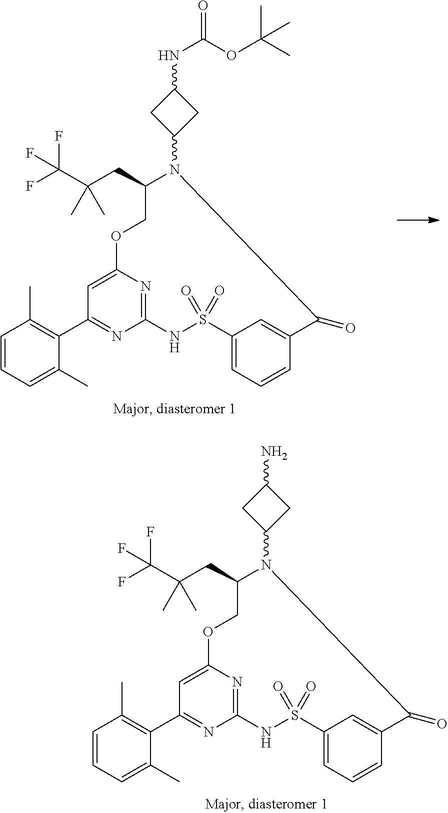 [SMILES: Cc1cccc(C)c1-c1cc2nc(n1)NS(=O)(=O)c1cccc(c1)C(=O)N(C1CC(NC(=O)OC(C)(C)C)C1)[C@H](CC(C)(C)C(F)(F)F)CO2 ; Cc1cccc(C)c1-c1cc2nc(n1)NS(=O)(=O)c1cccc(c1)C(=O)N(C1CC(N)C1)[C@H](CC(C)(C)C(F)(F)F)CO2]

A 100 mL flask containing tert-butyl N-{3-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]cyclobutyl}carbamate (310 mg, 0.4301 mmol) (more polar major isomer 1) was treated with DCM (4 mL) and HCl (3.5 mL of 4 M, 14.00 mmol) (4M in dioxane) at room temperature for 4 hour. The volatiles were removed. The residue was treated with DCM/hexanes and the solvents were removed by evaporation. The operation was repeated several times until a solid was obtained. (11R)-12-(3-aminocyclobutyl)-6-(2,6-dimethylphenyl)-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (hydrochloride salt) (299 mg, 96%) was isolated as an off-white solid. ESI-MS m/z calc. 617.22833, found 618.66 (M+1)⁺; Retention time: 1.16 minutes (LC method A).

### Step 4: Propan-2-yl N-{3-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]cyclobutyl}carbamate (Compound 192)

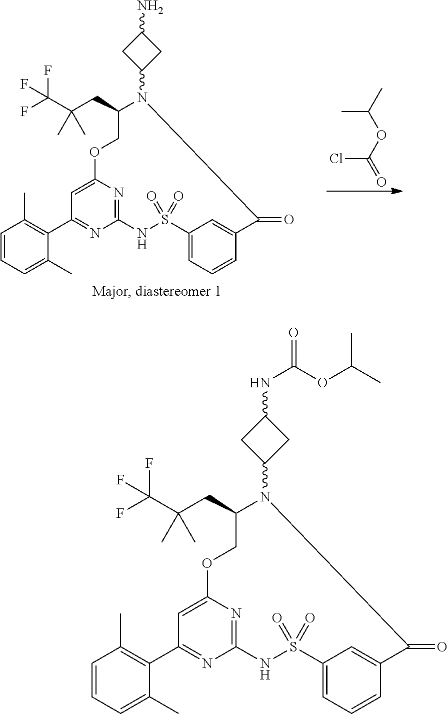

A 4 mL vial was charged with (11R)-12-(3-aminocyclobutyl)-6-(2,6-dimethylphenyl)-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (hydrochloride salt)(32 mg, 0.04892 mmol) (Major isomer, diastereomer 1), DCM (1 mL) and DIEA (42 μL, 0.2411 mmol). Isopropyl chloroformate (30 μL of 2 M, 0.06000 mmol) (2M toluene solution) was added and the mixture was stirred at room temperature for 4 hours (60% conversion). More isopropyl chloroformate (30 μL of 2 M, 0.06000 mmol) was added and the mixture was stirred for 1.5 hours. A bit of methanol was added, and the solvents were evaporated. DMSO (1 mL) was added. The solution was microfiltered through a syringe filter disc and purified by reverse phase preparative HPLC (C₁₈) using a gradient of acetonitrile in water (1 to 99% over 15 min) and HCl as a modifier to give propan-2-yl N-{3-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]cyclobutyl}carbamate (24.2 mg, 70%) as a white solid. ESI-MS m/z calc. 703.26514, found 704.74 (M+1)⁺; Retention time: 1.83 minutes. ¹H NMR (400 MHz, DMSO-d₆) δ 13.57-11.65 (broad m, 1H), 8.45 (s, 1H), 7.93 (s, 1H), 7.69 (s, 2H), 7.45 (d, J=7.1 Hz, 1H), 7.34-7.21 (m, 1H), 7.12 (d, J=7.6 Hz, 2H), 6.42 (br s, 1H), 5.14 (dd, J=11.0, 4.3 Hz, 1H), 4.76 (hept, J=6.3 Hz, 1H), 4.30-4.20 (m, 2H), 4.17-3.99 (m, 1H), 3.82 (br s, 1H), 3.28-3.05 (m, 2H), 2.31-1.65 (m, 10H), 1.18 (d, J=6.2 Hz, 6H), 0.83 (s, 3H), 0.60 (s, 3H), (LC method A).

### Example 153: Preparation of Compound 195

### Step 1: (11R)-12-(3-Aminocyclobutyl)-6-(2,6-dimethylphenyl)-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione, minor diastereomer 2

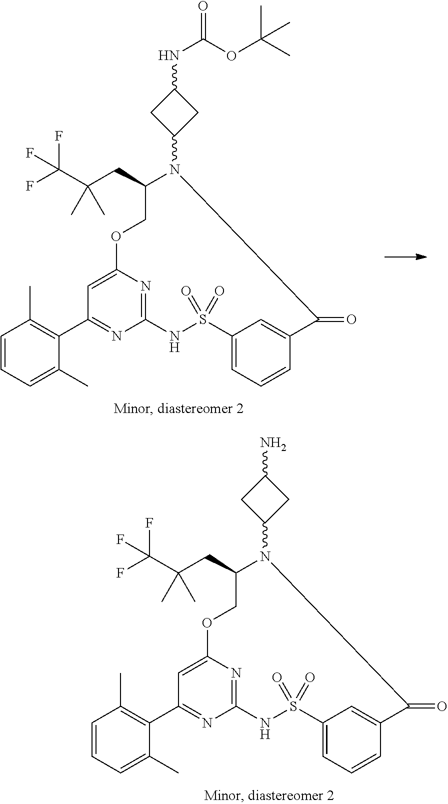 [SMILES: Cc1cccc(C)c1-c1cc2nc(n1)NS(=O)(=O)c1cccc(c1)C(=O)N(C1CC(NC(=O)OC(C)(C)C)C1)[C@H](CC(C)(C)C(F)(F)F)CO2 ; Cc1cccc(C)c1-c1cc2nc(n1)NS(=O)(=O)c1cccc(c1)C(=O)N(C1CC(N)C1)[C@H](CC(C)(C)C(F)(F)F)CO2]

A 100 mL flask containing tert-butyl N-{3-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]cyclobutyl}carbamate (124 mg, 0.1728 mmol) (less polar minor isomer B, diastereomer 2) was treated with DCM (1.6 mL) and HCl (1.4 mL of 4 M, 5.600 mmol) (4M in dioxane) at room temperature for 4 hour. The volatiles were removed. The residue was treated with DCM/hexanes and the solvents were removed by evaporation. The operation was repeated several times until a solid was obtained. (11R)-12-(3-aminocyclobutyl)-6-(2,6-dimethylphenyl)-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (hydrochloride salt), diastereomer 2 (129 mg, 98%) was isolated as an off-white solid. ESI-MS m/z calc. 617.22833, found 618.66 (M+1)⁺; Retention time: 1.21 minutes. ESI-MS m/z calc. 617.22833, found 618.66 (M+1)⁺; Retention time: 1.21 minutes (LC method A).

### Step 2: propan-2-yl N-{3-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]cyclobutyl}carbamate (Compound 195)

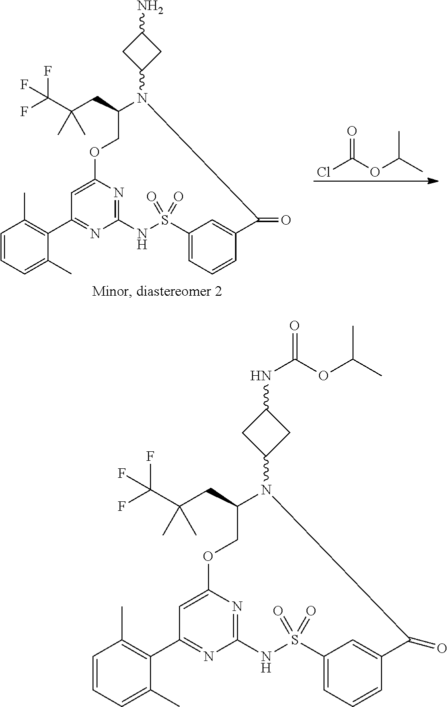

A 4 mL vial was charged with (11R)-12-(3-aminocyclobutyl)-6-(2,6-dimethylphenyl)-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (hydrochloride salt) (30 mg, 0.03944 mmol) (minor diastereomer 2), DCM (1 mL) and DIEA (34 μL, 0.1952 mmol). Isopropyl chloroformate (24 μL of 2 M, 0.04800 mmol) (2M toluene solution) was added and the mixture was stirred at room temperature for 4 hour (60% conversion). More isopropyl chloroformate (24 μL of 2 M, 0.04800 mmol) was added and the mixture was stirred for 1.5 hour. A bit of methanol was added, and the solvents were evaporated. DMSO (1 mL) was added. The solution was microfiltered through a syringe filter disc and purified by reverse phase preparative HPLC (C₁₈) using a gradient of acetonitrile in water (1 to 99% over 15 min) and HCl as a modifier to give propan-2-yl N-{3-[(11R)-6-(2,6-dimethylphenyl)-2,2,13-trioxo-11-(3,3,3-trifluoro-2,2-dimethylpropyl)-9-oxa-2λ⁶-thia-3,5,12,19-tetraazatricyclo[12.3.1.14,8]nonadeca-1(17),4(19),5,7,14(18),15-hexaen-12-yl]cyclobutyl}carbamate (20.2 mg, 73%) as a white solid. ESI-MS m/z calc. 703.26514, found 704.67 (M+1)⁺; Retention time: 1.86 minutes (LC method A). ¹H NMR (400 MHz, DMSO-d₆) δ 13.54-11.69 (broad m, 1H), 8.45 (s, 1H), 7.93 (s, 1H), 7.68 (s, 2H), 7.41-7.19 (m, 2H), 7.12 (d, J=7.5 Hz, 2H), 6.40 (br s, 1H), 5.16 (dd, J=11.0, 4.3 Hz, 1H), 4.74 (p, J=6.2 Hz, 1H), 4.34 (t, J=11.2 Hz, 1H), 3.89-3.66 (m, 3H), 2.78 (q, J=10.0 Hz, 2H), 2.52 (m, 2H, overlapped with DMSO), 2.27-1.81 (m, 7H), 1.75 (d, J=15.7 Hz, 1H), 1.16 (d, J=6.2 Hz, 6H), 0.86 (s, 3H), 0.64 (s, 3H).

### Example 154: Preparation of Compound 196

### Step 1: 4-Chloro-6-(2,6-dimethylphenyl)pyridin-2-amine

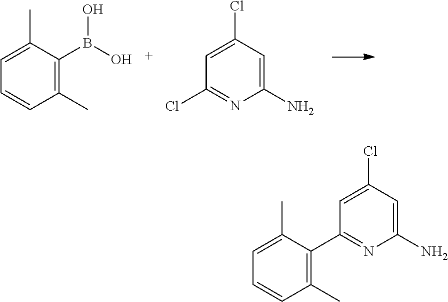 [SMILES: Cc1cccc(C)c1B(O)O ; Nc1cc(Cl)cc(Cl)n1 ; Cc1cccc(C)c1-c1cc(Cl)cc(N)n1]

To a stirring solution of (2,6-dimethylphenyl)boronic acid (11.515 g, 76.775 mmol) and 4,6-dichloropyridin-2-amine (12.513 g, 76.765 mmol) in Toluene (425 mL) and EtOH (213 mL) was added an aqueous solution of Sodium carbonate (115 mL of 2 M, 230.00 mmol) and the reaction mixture was degassed with nitrogen gas for 45 min. Pd(dppf)Cl₂ (6.271 g, 7.6791 mmol) was then added with degassing continuing for an additional 15 min. Then the reaction vial was sealed, and the mixture heated to 100° C. and stirred at that temperature for 24 h. After this time, volatiles were removed under reduced pressure and the residue was extracted with ethyl acetate (3×200 mL). The combined organic layers were washed with brine, dried over anhydrous sodium sulfate and concentrated under reduced pressure. The crude product was purified by silica gel column chromatography (0-25% EtOAc in Hexanes) and triturated with hexanes to afford 4-chloro-6-(2,6-dimethylphenyl)pyridin-2-amine (6.469 g, 34%) as an off-white solid. ESI-MS m/z calc. 232.07672, found 233.1 (M+1)⁺; Retention time: 2.31 minutes; (LC method T).

### Step 2 Methyl 3-[[4-chloro-6-(2,6-dimethylphenyl)-2-pyridyl]sulfamoyl]benzoate

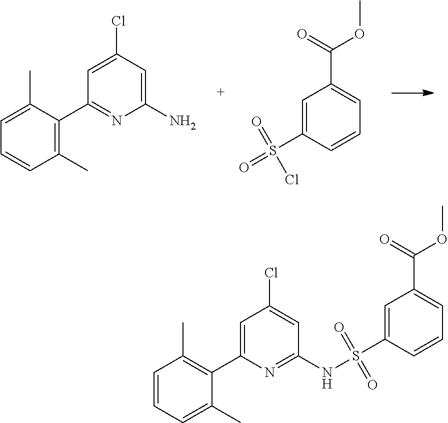 [SMILES: Cc1cccc(C)c1-c1cc(Cl)cc(N)n1 ; COC(=O)c1cccc(S(=O)(=O)Cl)c1 ; COC(=O)c1cccc(S(=O)(=O)Nc2cc(Cl)cc(-c3c(C)cccc3C)n2)c1]

To a solution of 4-chloro-6-(2,6-dimethylphenyl)pyridin-2-amine (4.9 g, 20.635 mmol) and methyl 3-chlorosulfonylbenzoate (4.9 g, 20.046 mmol) in THE (200 mL) was added dropwise lithium bis(trimethylsilyl)amide (45 mL of 1 M, 45.000 mmol) at −78° C. under nitrogen. The reaction mixture was stirred for 30 minutes at −78° C.; then warmed up to 0° C. and stirred for 2 hours at 0° C. The reaction was quenched with cold 1.0 M Hydrochloric acid (50 mL) and diluted with water (200 mL). The mixture was extracted with ethyl acetate (2×400 mL). The organic layers were combined, washed with brine (500 mL), dried over sodium sulfate, filtered and concentrated. The residue was purified by chromatography using 0-20% ethyl acetate in hexanes to afford methyl 3-[[4-chloro-6-(2,6-dimethylphenyl)-2-pyridyl]sulfamoyl]benzoate (6.2 g, 68%) as a white solid. ESI-MS m/z calc. 430.0754, found 431.5 (M+1)⁺; Retention time: 3.65 minutes; (LC method T).

### Step 3: 3-[[4-chloro-6-(2,6-dimethylphenyl)-2-pyridyl]sulfamoyl]benzoic acid

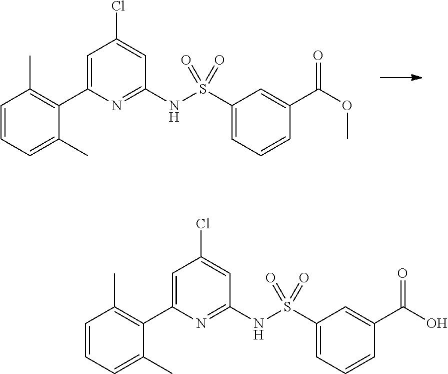 [SMILES: COC(=O)c1cccc(S(=O)(=O)Nc2cc(Cl)cc(-c3c(C)cccc3C)n2)c1 ; Cc1cccc(C)c1-c1cc(Cl)cc(NS(=O)(=O)c2cccc(C(=O)O)c2)n1]

To a stirring solution of 3-[4-chloro-6-(2,6-dimethyl-phenyl)-pyridin-2-ylsulfamoyl]-benzoic acid methyl ester (5.3 g, 12.3 mmol) in a mixture of tetrahydrofuran (80 mL) and water (80 mL) at room temperature was added lithium hydroxide monohydrate (1.55 g, 36.9 mmol) and the reaction mixture was stirred at 45° C. for 2 hours. Tetrahydrofuran was removed under vacuum and the residue was diluted with water (100 mL). The aqueous layer was washed with diethyl ether (2×50 mL), hexanes (50 mL) and acidified with 1.0 M hydrochloric acid to pH=2-3. The precipitated product was collected by filtration and dried in a vacuum oven at 75° C. to constant weight to afford 3-[4-chloro-6-(2,6-dimethyl-phenyl)-pyridin-2-ylsulfamoyl]-benzoic acid (4.8 g, 93%) as a white solid. H NMR (250 MHz, DMSO-d₆) δ (ppm): 8.32 (d, J=1.9 Hz, 1H), 8.14 (d, J=7.7 Hz, 1H), 8.03 (d, J=8.0 Hz, 1H), 7.63 (t, J=7.8 Hz, 1H), 7.28-6.96 (m, 5H), 1.77 (s, 6H). ESI-MS m/z calc. 416.8, found 417.0 (M1). Retention time: 5.11 minutes.

### Step 4: 3-[[4-[(2R)-2-Amino-4-methyl-pentoxy]-6-(2,6-dimethylphenyl)-2-pyridyl]sulfamoyl]benzoic acid

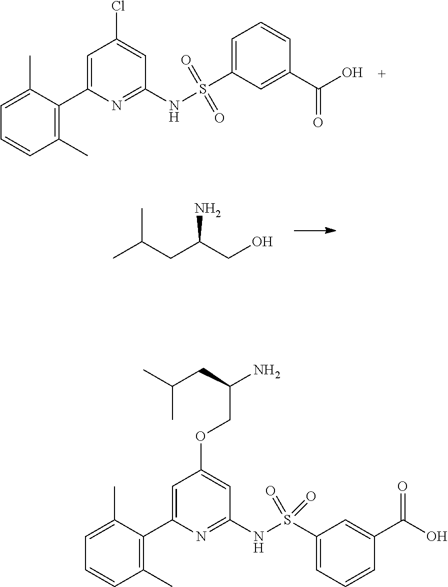

A 20 mL vial was charged with 3-[[4-chloro-6-(2,6-dimethylphenyl)-2-pyridyl]sulfamoyl]benzoic acid (300 mg, 0.7196 mmol), (2R)-2-amino-4-methyl-pentan-1-ol (110 mg, 0.9387 mmol) and anhydrous tetrahydrofuran (12 mL), in that order. Then the vial was purged with nitrogen for 30 seconds, and solid potassium tert-butoxide (350 mg, 3.119 mmol) was added capped under nitrogen. After stirred at 105° C. for 14 h (overnight), the reaction was allowed to cool to ambient temperature. Then glacial acetic acid (200 μL, 3.517 mmol) was added and the volatiles were removed under reduced pressure. To the residue, DMSO (5 mL) was added and microfiltered. Purification by reverse phase chromatography (Cis column, 1-99% acetonitrile in water over 15 min) gave 3-[[4-[(2R)-2-amino-4-methyl-pentoxy]-6-(2,6-dimethylphenyl)-2-pyridyl]sulfamoyl]benzoic acid (hydrochloride salt)(278 mg, 72%) as yellowish solid. ESI-MS m/z calc. 497.19846, found 498.2 (M+1)⁺; Retention time: 0.43 minutes (LC method D).

### Step 5: 3-[[4-[(2R)-2-[(7-tert-Butoxycarbonyl-7-azaspiro[3.5]nonan-2-yl)amino]-4-methyl-pentoxy]-6-(2,6-dimethylphenyl)-2-pyridyl]sulfamoyl]benzoic acid

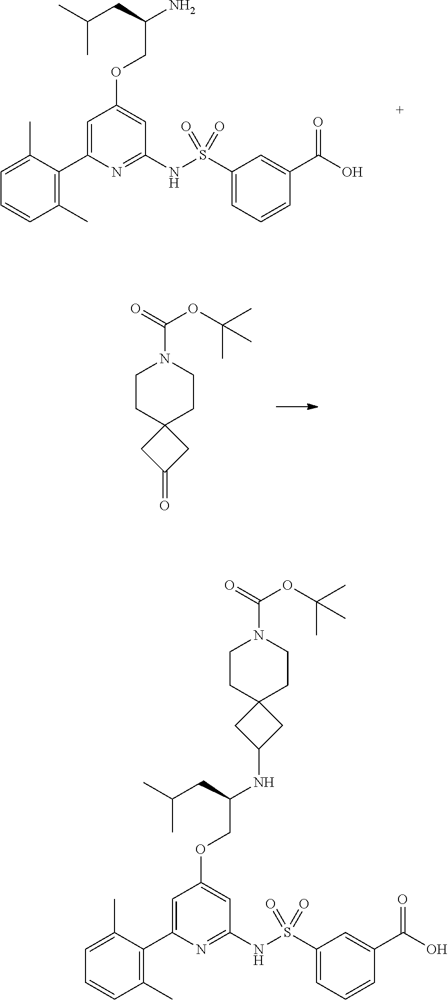 [SMILES: Cc1cccc(C)c1-c1cc(OC[C@H](N)CC(C)C)cc(NS(=O)(=O)c2cccc(C(=O)O)c2)n1 ; CC(C)(C)OC(=O)N1CCC2(CC1)CC(=O)C2 ; Cc1cccc(C)c1-c1cc(OC[C@@H](CC(C)C)NC2CC3(CCN(C(=O)OC(C)(C)C)CC3)C2)cc(NS(=O)(=O)c2cccc(C(=O)O)c2)n1]

In a 50 mL round-bottom flask, to a stirred heterogeneous mixture of 3-[[4-[(2R)-2-amino-4-methyl-pentoxy]-6-(2,6-dimethylphenyl)-2-pyridyl]sulfamoyl]benzoic acid (hydrochloride salt) (300 mg, 0.6029 mmol) and tert-butyl 2-oxo-7-azaspiro[3.5]nonane-7-carboxylate (300 mg, 1.254 mmol) in anhydrous 1,2-dichloro-ethane (6 mL) were added triethylamine (250 μL, 1.794 mmol) and glacial acetic acid (50 μL, 0.8792 mmol), in that order, at ambient temperature. The tan solution was stirred under nitrogen at room ambient temperature for about 30 min. Then sodium triacetoxyborohydride (500 mg, 2.359 mmol) was added and stirred continued at room temperature overnight (total 14 h). Then methanol (2 mL) was added and the volatiles were removed under reduced pressure. The residue was taken up in DMSO (5 mL). The solution was microfiltered and purified by reverse phase preparative chromatography (C₁₈) using 1-99% acetonitrile in water (over 15 min) and HCl as a modifier. Genevac evaporation of the desired fractions afforded desired 3-[[4-[(2R)-2-[(7-tert-butoxycarbonyl-7-azaspiro[3.5]nonan-2-yl)amino]-4-methyl-pentoxy]-6-(2,6-dimethylphenyl)-2-pyridyl]sulfamoyl]benzoic acid (271 mg, 62%) as white solid. ¹H NMR (500 MHz, DMSO-d₆) δ 8.39 (s, 1H), 8.08 (d, J=7.7 Hz, 1H), 8.00 (d, J=7.8 Hz, 1H), 7.62 (t, J=7.6 Hz, 1H), 7.25 (d, J=8.0 Hz, 2H), 7.18 (d, J=7.6 Hz, 1H), 7.11 (d, J=7.4 Hz, 2H), 6.73 (s, 1H), 6.44-6.38 (m, 1H), 4.02 (s, 2H), 3.23-3.19 (m, 2H), 3.16-3.09 (m, 2H), 3.01-2.93 (m, 1H), 1.98 (s, 6H), 1.77-1.68 (m, 1H), 1.61-1.46 (m, 2H), 1.45-1.40 (m, 2H), 1.39 (s, 9H), 1.35-1.28 (m, 2H), 0.88 (d, J=6.6 Hz, 3H), 0.85 (d, J=6.4 Hz, 3H). ESI-MS m/z calc. 720.35565, found 721.5 (M+1)⁺; Retention time: 0.58 minutes, LC method D.

### Step 6: tert-Butyl 2-[(11R)-6-(2,6-dimethylphenyl)-11-isobutyl-2,2,13-trioxo-9-oxa-2λ⁶-thia-3,5,12-triazatricyclo[12.3.1.14,8]nonadeca-1(18),4(19),5,7,14,16-hexaen-12-yl]-7-azaspiro[3.5]nonane-7-carboxylate

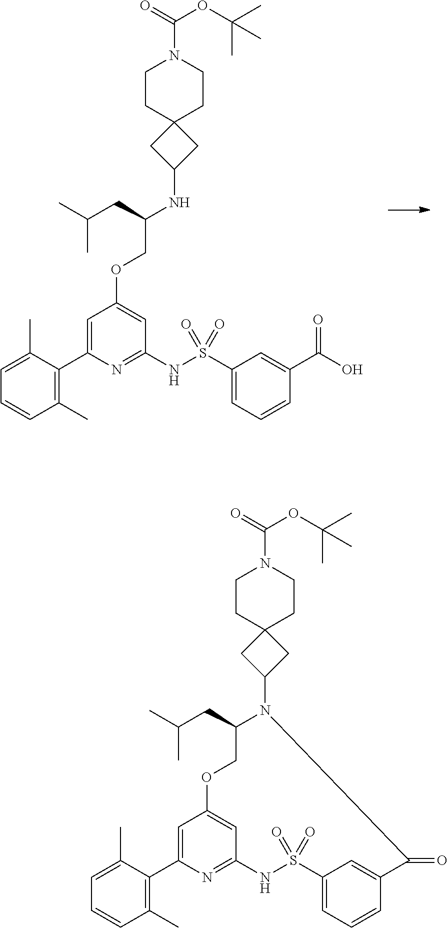 [SMILES: Cc1cccc(C)c1-c1cc(OC[C@@H](CC(C)C)NC2CC3(CCN(C(=O)OC(C)(C)C)CC3)C2)cc(NS(=O)(=O)c2cccc(C(=O)O)c2)n1 ; Cc1cccc(C)c1-c1cc2cc(n1)NS(=O)(=O)c1cccc(c1)C(=O)N(C1CC3(CCN(C(=O)OC(C)(C)C)CC3)C1)[C@H](CC(C)C)CO2]

In a 50 mL round-bottom flask, to a stirred solution of 3-[[4-[(2R)-2-[(7-tert-butoxycarbonyl-7-azaspiro[3.5]nonan-2-yl)amino]-4-methyl-pentoxy]-6-(2,6-dimethylphenyl)-2-pyridyl]sulfamoyl]benzoic acid (250 mg, 0.3468 mmol) and N,N-diisopropyl ethyl amine (400 μL, 2.296 mmol) in anhydrous DMF (10 mL) was added [dimethylamino(triazolo[4,5-b]pyridin-3-yloxy)methylene]-dimethyl-ammonium (Phosphorus Hexafluoride Ion) (200 mg, 0.5260 mmol) (HATU) at ambient temperature under nitrogen. The tea-colored solution was stirred at ambient temperature for 14 h (overnight). The crude reaction mixture was microfiltered and purified from preparative reverse-phase HPLC (Cis column, 1-99% acetonitrile in water over 15 min, HCl as modifier). The desired fractions were combined and concentrated under reduced pressure to half of the original volume. Then extracted with ethyl acetate (3×30 mL). The combined organics were washed with brine (20 mL), dried over anhydrous sodium sulfate, filtered and concentrated under reduced pressure to afford tert-butyl 2-[(11R)-6-(2,6-dimethylphenyl)-11-isobutyl-2,2,13-trioxo-9-oxa-2λ⁶-thia-3,5,12-triazatricyclo[12.3.1.14,8]nonadeca-1(18),4(19),5,7,14,16-hexaen-12-yl]-7-azaspiro[3.5]nonane-7-carboxylate (40 mg, 16%) as a white solid. ESI-MS m/z calc. 702.3451, found 703.3 (M+1)⁺; Retention time: 0.83 minutes. LC method D.

### Step 7: (11R)-12-(7-Azaspiro[3.5]nonan-2-yl)-6-(2,6-dimethylphenyl)-11-isobutyl-2,2-dioxo-9-oxa-2λ⁶-thia-3,5,12-triazatricyclo[12.3.1.14,8]nonadeca-1(18),4(19),5,7,14,16-hexaen-13-one

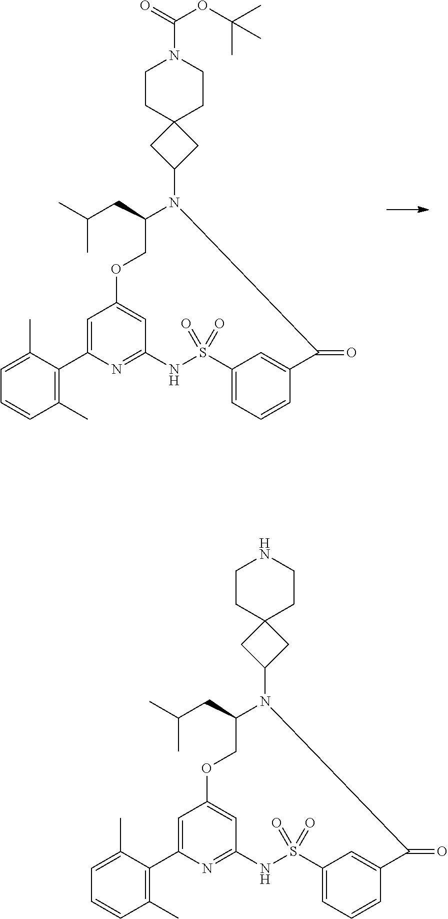 [SMILES: Cc1cccc(C)c1-c1cc2cc(n1)NS(=O)(=O)c1cccc(c1)C(=O)N(C1CC3(CCN(C(=O)OC(C)(C)C)CC3)C1)[C@H](CC(C)C)CO2 ; Cc1cccc(C)c1-c1cc2cc(n1)NS(=O)(=O)c1cccc(c1)C(=O)N(C1CC3(CCNCC3)C1)[C@H](CC(C)C)CO2]

To a stirred solution of tert-butyl 2-[(11R)-6-(2,6-dimethylphenyl)-11-isobutyl-2,2,13-trioxo-9-oxa-2λ⁶-thia-3,5,12-triazatricyclo[12.3.1.14,8]nonadeca-1(18),4(19),5,7,14,16-hexaen-12-yl]-7-azaspiro[3.5]nonane-7-carboxylate (36 mg, 0.05122 mmol) in anhydrous dichloromethane (2 mL) was added 4 M hydrogen chloride in dioxane (350 μL of 4.0 M, 1.400 mmol) under nitrogen at ambient temperature. The pale-yellow solution was stirred for 30 min, then concentrated under reduced pressure and dried further in vacuo to give (11R)-12-(7-azaspiro[3.5]nonan-2-yl)-6-(2,6-dimethylphenyl)-11-isobutyl-2,2-dioxo-9-oxa-2λ⁶-thia-3,5,12-triazatricyclo[12.3.1.14,8]nonadeca-1(18),4(19),5,7,14,16-hexaen-13-one (hydrochloride salt) (33 mg, 101%) as white solid. ESI-MS m/z calc. 602.29266, found 603.5 (M+1)⁺; Retention time: 1.3 minutes, LC method A.

### Step 8: Methyl 2-[(11R)-6-(2,6-dimethylphenyl)-11-isobutyl-2,2,13-trioxo-9-oxa-2λ⁶-thia-3,5,12-triazatricyclo[12.3.1.14,8]nonadeca-1(18),4(19),5,7,14,16-hexaen-12-yl]-7-azaspiro[3.5]nonane-7-carboxylate (Compound 196)

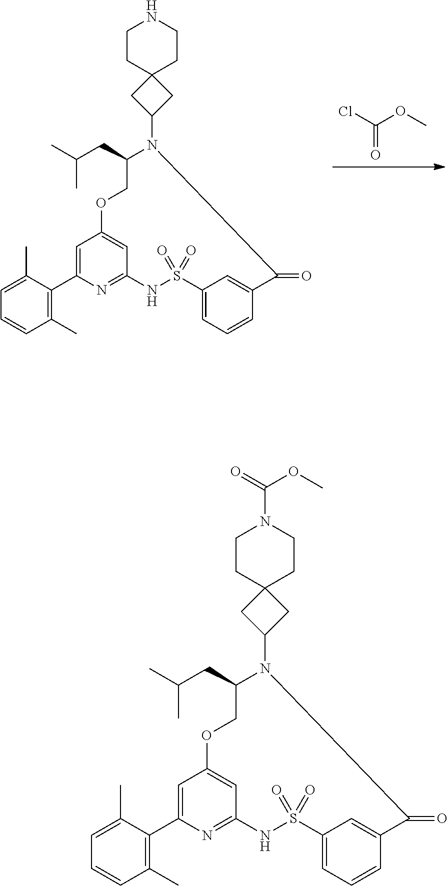

To a stirred solution of (11R)-12-(7-azaspiro[3.5]nonan-2-yl)-6-(2,6-dimethylphenyl)-11-isobutyl-2,2-dioxo-9-oxa-2λ⁶-thia-3,5,12-triazatricyclo[12.3.1.14,8]nonadeca-1(18),4(19),5,7,14,16-hexaen-13-one (hydrochloride salt) (32 mg, 0.05006 mmol) and triethylamine (30 μL, 0.2152 mmol) in anhydrous methylene chloride (1.0 mL) was added a solution of methyl chloroformate (6 mg, 0.06349 mmol) in anhydrous methylene chloride (0.5 mL) over 1 min, while maintaining a temperature between 0° C. and 5° C. (ice-water bath), and the mixture was stirred at that temperature for 2 h. The reaction was concentrated under reduced pressure and the residue was taken up in DMSO (1 mL). The solution was microfiltered and purified by preparative reverse-phase HPLC to give methyl 2-[(11R)-6-(2,6-dimethylphenyl)-11-isobutyl-2,2,13-trioxo-9-oxa-2λ⁶-thia-3,5,12-triazatricyclo[12.3.1.14,8]nonadeca-1(18),4(19),5,7,14,16-hexaen-12-yl]-7-azaspiro[3.5]nonane-7-carboxylate (18.2 mg, 54%) as colorless solid. ESI-MS m/z calc. 660.29816, found 661.5 (M+1)⁺; Retention time: 1.86 minutes, LC method A.

### Example 155: Preparation of Compound 197

### Step 1: 6-(2,6-Dimethylphenyl)-4-[(2R)-4-methyl-2-(spiro[2.3]hexan-5-ylamino)pentoxy]pyridin-2-amine

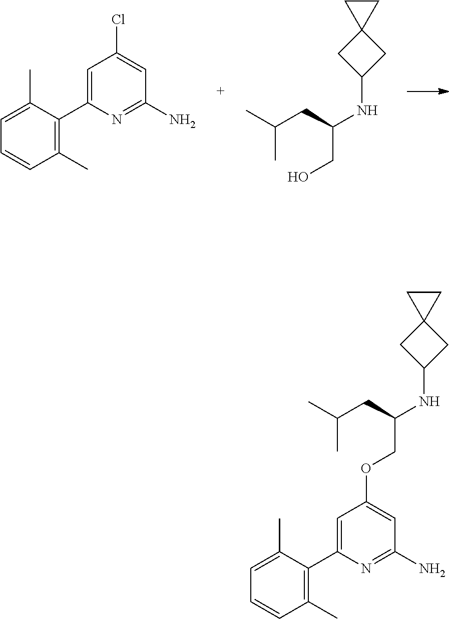 [SMILES: Cc1cccc(C)c1-c1cc(Cl)cc(N)n1 ; CC(C)C[C@H](CO)NC1CC2(CC2)C1 ; Cc1cccc(C)c1-c1cc(OC[C@@H](CC(C)C)NC2CC3(CC3)C2)cc(N)n1]

In a 20 mL microwave tube, to a stirred solution of 4-chloro-6-(2,6-dimethylphenyl)pyridin-2-amine (400 mg, 1.719 mmol) in anhydrous dimethylsulfoxide (5 mL) were added (2R)-4-methyl-2-(spiro[2.3]hexan-5-ylamino)pentan-1-ol (hydrochloride salt) (450 mg, 1.925 mmol) and powdered potassium hydroxide (500 mg, 8.912 mmol), in that order, at ambient temperature. The heterogeneous mixture was purged with nitrogen for 30 sec, capped and stirred at 95° C. overnight (14 h). Then stirring continued at 112° C. for 6 h. The reaction mixture was allowed to cool to ambient temperature and acidified with glacial acetic acid (600 μL, 10.55 mmol), filtered through a short plug of Celite, then micro-filtered and purified by reverse-phase HPLC (Cis column, 1-99% acetonitrile in water over 15 min, HCl as modifier) to afford 6-(2,6-dimethylphenyl)-4-[(2R)-4-methyl-2-(spiro[2.3]hexan-5-ylamino)pentoxy]pyridin-2-amine (Dihydrochloride salt) (507 mg, 63%) as pale-yellow solid. ¹H NMR (500 MHz, DMSO-d₆) δ 13.73 (s, 1H), 9.79 (s, 1H), 9.69 (s, 1H), 7.93 (s, 2H), 7.35 (t, J=7.6 Hz, 1H), 7.22 (d, J=7.6 Hz, 2H), 6.58 (d, J=2.3 Hz, 1H), 6.51 (d, J=2.4 Hz, 1H), 4.48 (dd, J=11.3, 3.2 Hz, 1H), 4.42 (dd, J=11.2, 4.9 Hz, 1H), 4.06 (s, 1H), 3.46 (s, 1H), 2.67 (dd, J=11.5, 7.8 Hz, 2H), 2.24-2.19 (m, 1H), 2.18 (s, 6H), 2.17-2.12 (m, 1H), 1.84-1.74 (m, 1H), 1.74-1.60 (m, 2H), 0.94 (d, J=6.6 Hz, 3H), 0.93 (d, J=6.7 Hz, 3H), 0.54-0.48 (m, 2H), 0.48-0.39 (m, 2H). ESI-MS m/z calc. 393.278, found 394.3 (M+1)⁺; Retention time: 1.01 minutes (LC method A).

### Step 2: Methyl 6-[[6-(2,6-dimethylphenyl)-4-[(2R)-4-methyl-2-(spiro[2.3]hexan-5-ylamino)pentoxy]-2-pyridyl]sulfamoyl]pyridine-2-carboxylate

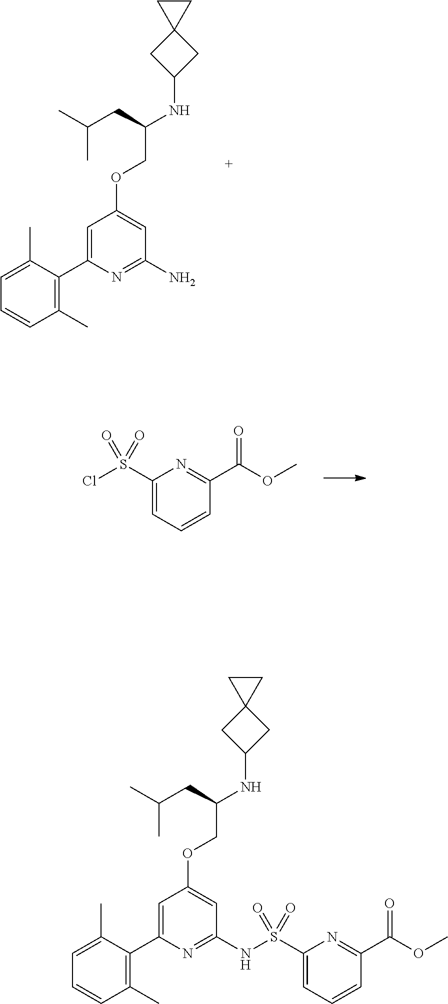 [SMILES: Cc1cccc(C)c1-c1cc(OC[C@@H](CC(C)C)NC2CC3(CC3)C2)cc(N)n1 ; COC(=O)c1cccc(S(=O)(=O)Cl)n1 ; COC(=O)c1cccc(S(=O)(=O)Nc2cc(OC[C@@H](CC(C)C)NC3CC4(CC4)C3)cc(-c3c(C)cccc3C)n2)n1]

In a 25 mL round-bottom flask, to a stirred solution of 6-(2,6-dimethylphenyl)-4-[(2R)-4-methyl-2-(spiro[2.3]hexan-5-ylamino)pentoxy]pyridin-2-amine (Dihydrochloride salt) (100 mg, 0.2325 mmol) in anhydrous pyridine (5 mL) was added solid methyl 6-chlorosulfonylpyridine-2-carboxylate (66 mg, 0.2801 mmol) in two portions at room temperature under nitrogen. Stirring continued overnight (16 h) at that temperature. The solvent was removed under reduced pressure, and to the thick brownish residue toluene (20 mL) was added and removed under reduced pressure to remove the residual pyridine present. Then repeated the last process twice with toluene. The thick residue was triturated with methylene chloride (15 mL), filtered (to remove white pyridine hydrochloride salt). The filtrate was concentrated under reduced pressure and the residue was taken up in DMSO (1.5 mL), micro-filtered and purified by reverse-phase HPLC (Cis column, 1-99% acetonitrile in water over 15 min, HCl as modifier) to afford methyl 6-[[6-(2,6-dimethylphenyl)-4-[(2R)-4-methyl-2-(spiro[2.3]hexan-5-ylamino)pentoxy]-2-pyridyl]sulfamoyl]pyridine-2-carboxylate (hydrochloride salt) (89 mg, 61%) as off-white solid. ¹H NMR (400 MHz, DMSO-d₆) δ 9.15 (s, 2H), 8.30-8.08 (m, 3H), 7.27 (s, 2H), 7.16-6.94 (m, 2H), 6.46 (s, 1H), 4.50-4.28 (m, 2H), 4.14-3.98 (m, 1H), 3.89 (s, 3H), 3.46 (s, 1H), 2.52 (t, J=6.7 Hz, 1H), 2.26-2.14 (m, 2H), 2.06 (s, 6H), 1.80-1.61 (m, 3H), 1.59-1.46 (m, 1H), 0.90 (d, J=6.2 Hz, 6H), 0.52-0.45 (m, 2H), 0.44-0.34 (m, 2H). ESI-MS m/z calc. 592.2719, found 593.5 (M+1)⁺; Retention time: 0.53 minutes (LC method D).

### Step 3: 6-[[6-(2,6-Dimethylphenyl)-4-[(2R)-4-methyl-2-(spiro[2.3]hexan-5-ylamino)pentoxy]-2-pyridyl]sulfamoyl]pyridine-2-carboxylic acid

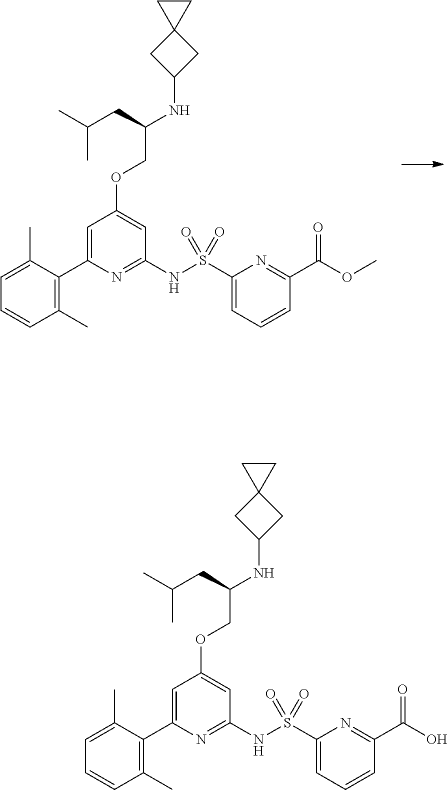 [SMILES: COC(=O)c1cccc(S(=O)(=O)Nc2cc(OC[C@@H](CC(C)C)NC3CC4(CC4)C3)cc(-c3c(C)cccc3C)n2)n1 ; Cc1cccc(C)c1-c1cc(OC[C@@H](CC(C)C)NC2CC3(CC3)C2)cc(NS(=O)(=O)c2cccc(C(=O)O)n2)n1]

In a 25 mL flask, to a stirred solution of methyl 6-[[6-(2,6-dimethylphenyl)-4-[(2R)-4-methyl-2-(spiro[2.3]hexan-5-ylamino)pentoxy]-2-pyridyl]sulfamoyl]pyridine-2-carboxylate (hydrochloride salt) (80 mg, 0.1271 mmol) in tetrahydrofuran (1.5 mL) and methanol (1.5 mL) was added sodium hydroxide (700 μL of 1.0 M, 0.7000 mmol) at once at ambient temperature. After 20 min, the reaction mixture was acidified with glacial acetic acid (50 μL, 0.8792 mmol) and the resultant heterogeneous mixture was concentrated under reduced pressure. The residue was taken up in DMSO (2 mL), micro-filtered and purified by reverse-phase HPLC (Cis column, 1-99% acetonitrile in water over 15 min, HCl as modifier) to afford 6-[[6-(2,6-dimethylphenyl)-4-[(2R)-4-methyl-2-(spiro[2.3]hexan-5-ylamino)pentoxy]-2-pyridyl]sulfamoyl]pyridine-2-carboxylic acid (hydrochloride salt) (61 mg, 78%) as off-white solid. ¹H NMR (500 MHz, DMSO-d₆) δ 9.39 (s, 1H), 9.30 (s, 1H), 8.20-8.12 (m, 3H), 7.26 (t, J=7.6 Hz, 1H), 7.13 (d, J=7.6 Hz, 3H), 6.48 (d, J=2.3 Hz, 1H), 4.41 (d, J=11.3 Hz, 1H), 4.34 (dd, J=11.3, 4.8 Hz, 1H), 4.09-4.04 (m, 1H), 3.50-3.42 (m, 1H), 2.58 (dt, J=11.9, 7.0 Hz, 2H), 2.25-2.18 (m, 1H), 2.18-2.11 (m, 1H), 2.01 (s, 6H), 1.79-1.66 (m, 2H), 1.65-1.57 (m, 1H), 0.93 (d, J=6.6 Hz, 3H), 0.92 (d, J=6.6 Hz, 3H), 0.49 (dt, J=8.4, 3.3 Hz, 2H), 0.47-0.40 (m, 2H). ESI-MS m/z calc. 578.2563, found 579.5 (M+1)⁺; Retention time: 1.21 minutes (LC method A).

### Step 4: (11R)-6-(2,6-Dimethylphenyl)-11-isobutyl-2,2-dioxo-12-spiro[2.3]hexan-5-yl-9-oxa-2λ⁶-thia-3,5,12,18-tetrazatricyclo[12.3.1.14,8]nonadeca-1(18),4(19),5,7,14,16-hexaen-13-one (Compound 197)

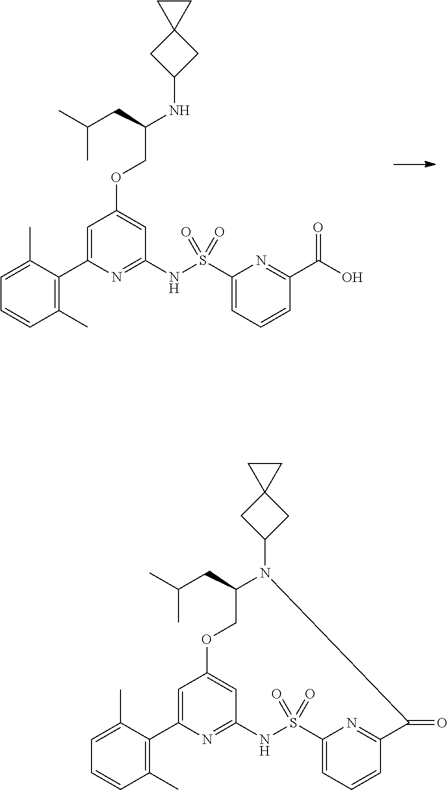 [SMILES: Cc1cccc(C)c1-c1cc(OC[C@@H](CC(C)C)NC2CC3(CC3)C2)cc(NS(=O)(=O)c2cccc(C(=O)O)n2)n1 ; Cc1cccc(C)c1-c1cc2cc(n1)NS(=O)(=O)c1cccc(n1)C(=O)N(C1CC3(CC3)C1)[C@H](CC(C)C)CO2]

In a 25 mL vial, to a stirred solution of 6-[[6-(2,6-dimethylphenyl)-4-[(2R)-4-methyl-2-(spiro[2.3]hexan-5-ylamino)pentoxy]-2-pyridyl]sulfamoyl]pyridine-2-carboxylic acid (hydrochloride salt) (50 mg, 0.08128 mmol) in anhydrous DMF (3 mL) were added [dimethylamino(triazolo[4,5-b]pyridin-3-yloxy)methylene]-dimethyl-ammonium (Phosphorus Hexafluoride Ion) (40 mg, 0.1052 mmol) (HATU) and DIEA (100 μL, 0.5741 mmol), in that order, Nitrogen gas was purged for 20 sec and capped under nitrogen. The reaction was stirred at ambient temperature for 2 h. The reaction mixture was concentrated to about 1 mL under reduced pressure, then diluted with DMSO (1 ML), micro-filtered and purified by reverse-phase HPLC (Cis column, 1-99% acetonitrile in water over 15 min, HCl as modifier) to give impure material (36 mg, 92% in UV220.100% in UV254). The material was purified again under the same conditions with a 30 min run to afford (11R)-6-(2,6-dimethylphenyl)-11-isobutyl-2,2-dioxo-12-spiro[2.3]hexan-5-yl-9-oxa-2λ⁶-thia-3,5,12,18-tetrazatricyclo[12.3.1.14,8]nonadeca-1(18),4(19),5,7,14,16-hexaen-13-one (27 mg, 59%) as white solid. ¹H NMR (400 MHz, DMSO-d₆) δ 8.18 (t, J=7.8 Hz, 1H), 8.05 (dd, J=7.9, 1.0 Hz, 1H), 7.83 (d, J=7.6 Hz, 1H), 7.74 (d, J=2.4 Hz, 1H), 7.24 (t, J=7.6 Hz, 1H), 7.13 (d, J=7.6 Hz, 1H), 7.08 (d, J=7.6 Hz, 1H), 6.31 (d, J=2.1 Hz, 1H), 4.70-4.55 (m, 2H), 4.28 (p, J=8.6 Hz, 1H), 3.96-3.81 (m, 1H), 3.36 (d, J=9.1 Hz, 2H), 2.13 (d, J=9.4 Hz, 1H), 2.08 (s, 3H), 2.02 (d, J=9.5 Hz, 1H), 1.85 (s, 3H), 1.62 (t, J=12.5 Hz, 1H), 1.34-1.20 (m, 1H), 1.11 (t, J=12.3 Hz, 1H), 0.69 (d, J=6.6 Hz, 3H), 0.55-0.49 (m, 2H), 0.48-0.42 (m, 2H), 0.02 (d, J=6.3 Hz, 3H). ESI-MS m z calc. 560.2457, found 561.4 (M+1)⁺; Retention time: 1.38 minutes (LC method A).

### Example 156: Preparation of Compound 198

### Step 1: (2R)-2-(Spiro[2.3]hexan-5-ylamino)-3-[1-(trifluoromethyl)cyclopropyl]propan-1-ol

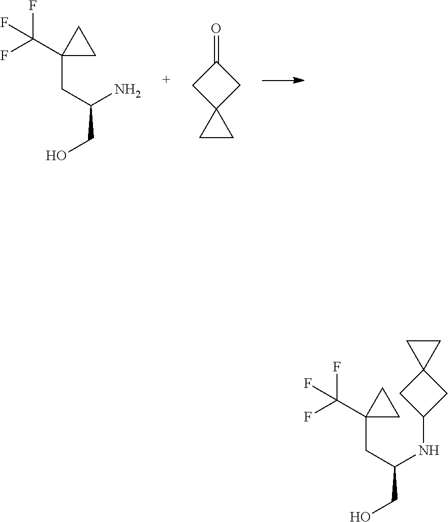 [SMILES: N[C@@H](CO)CC1(C(F)(F)F)CC1 ; O=C1CC2(CC2)C1 ; OC[C@@H](CC1(C(F)(F)F)CC1)NC1CC2(CC2)C1]

(2R)-2-Amino-3-[1-(trifluoromethyl)cyclopropyl]propan-1-ol (hydrochloride salt) (55 g, 250.4 mmol) was suspended in dichloroethane (825 mL), treated with spiro[2.3]hexan-5-one (26.6 g, 276.7 mmol) and stirred for 15 min (fine gelatinous suspension). Then sodium triacetoxyborohydride (158 g, 745.5 mmol) was added and the fine cream suspension was stirred at room temperature for 18 h. The thick suspension was cooled in an ice bath and quenched by slow addition of HCl (750 mL of 1 M, 750.0 mmol), keeping the internal temperature between 5 and 15° C. The emulsion (2 clear phases, water on top, pH=1) was stirred for 15 min and then made alkaline by addition of solid potassium carbonate (310 g, 2.243 mol) under cooling (internal temperature 5-15° C., strong foaming). Towards the end the DCE phase became a gelatinous suspension. MTBE (825 mL) was added to give an emulsion. The phases were separated, and the organic phase was washed once with saturated potassium carbonate. The aqueous phases were back extracted once with MTBE (75 ml) and the combined organic phases were dried, filtered and evaporated. The crude was dissolved in heptane (˜250 mL) under heating and the hot clear solution was left standing overnight. The resulting thick solid mass was stirred in an ice bath for 1 h. The solid was collected by filtration, washed with dry ice cold heptane and dried to give (2R)-2-(spiro[2.3]hexan-5-ylamino)-3-[1-(trifluoromethyl)cyclopropyl]propan-1-ol as an off-white solid (39 g, 59%). ¹H NMR (500 MHz, DMSO-d₆) δ 4.53 (t, J=5.2 Hz, 1H), 3.47 (p, J=7.4 Hz, 1H), 3.40 (dd, J=10.5, 4.8 Hz, 1H), 3.28 (dt, J=10.6, 5.1 Hz, 1H), 2.61 (p, J=5.8 Hz, 1H), 2.15-2.02 (m, 2H), 1.94 (ddd, J=9.9, 7.2, 3.6 Hz, 2H), 1.68 (dd, J=15.0, 6.4 Hz, 2H), 1.52 (dd, J=15.0, 7.1 Hz, 1H), 0.94-0.71 (m, 4H), 0.49-0.20 (m, 4H). ESI-MS m/z calc. 263.1497, found 264.0 (M+1)⁺; Retention time: 0.91 minutes (LC method A).

### Step 2: 3-[[4-(2,6-Dimethylphenyl)-6-[(2R)-2-(spiro[2.3]hexan-5-ylamino)-3-[1-(trifluoromethyl)cyclopropyl]propoxy]pyrimidin-2-yl]sulfamoyl]benzoic acid

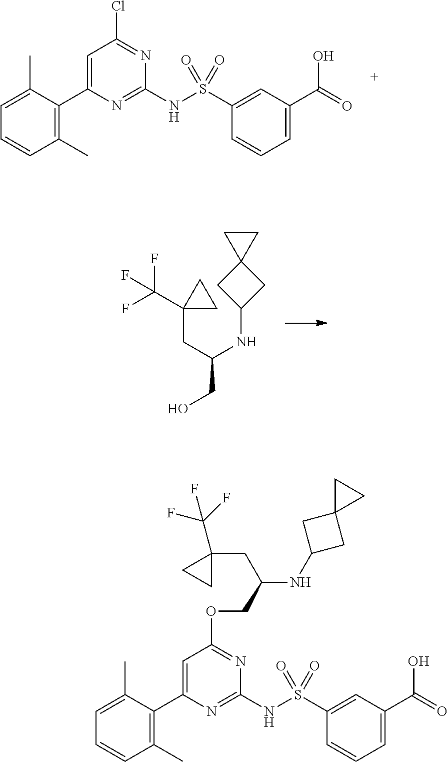 [SMILES: Cc1cccc(C)c1-c1cc(Cl)nc(NS(=O)(=O)c2cccc(C(=O)O)c2)n1 ; OC[C@@H](CC1(C(F)(F)F)CC1)NC1CC2(CC2)C1 ; Cc1cccc(C)c1-c1cc(OC[C@@H](CC2(C(F)(F)F)CC2)NC2CC3(CC3)C2)nc(NS(=O)(=O)c2cccc(C(=O)O)c2)n1]

(2R)-2-(Spiro[2.3]hexan-5-ylamino)-3-[1-(trifluoromethyl)cyclopropyl]propan-1-ol (38.5 g, 146.2 mmol) was dissolved in THE (1.2 L) (pale yellow solution) and purged with nitrogen (3×vacuum/N₂). The pale yellow solution was cooled in an acetone/ice bath and at an internal temperature of −2° C. NaOtBu (58 g, 585.4 mmol) was added quickly (over ˜1 min, only slightly exotherm, internal temperature up to 1° C.) against a weak nitrogen stream to give a pale yellow cloudy solution/fine suspension. Directly after the addition, 3-[[4-chloro-6-(2,6-dimethylphenyl)pyrimidin-2-yl]sulfamoyl]benzoic acid (61 g, 146.0 mmol) was added over 2-3 min. The added acid went quickly into solution and the internal temperature rose from 1 to 10° C. giving a yellow cloudy solution. As soon as the internal temperature started to drop again the ice bath was removed and the cloudy solution was stirred at room temperature for 2 h, then another portion of 3-[[4-chloro-6-(2,6-dimethylphenyl)pyrimidin-2-yl]sulfamoyl]benzoic acid (12 g, 28.72 mmol) was added and the reaction stirred at room temperature for another 45 minutes. The yellow cloudy solution was added to ice cold HCl (880 mL of 1 M, 880.0 mmol) under stirring and diluted with ethyl acetate (1.2 L). The phases were separated, and the organic phase was washed twice with brine (2×250 ml). The aqueous phases were back extracted once with ethyl acetate (250 ml). The combined organic phases were dried over magnesium sulfate, filtered (done quickly since product HCl salt crystallizes from ethyl acetate) and the yellow clear solution was evaporated to give a yellow foam/solid (˜125 g). The crude was triturated with ethyl acetate and stirred and the solid was collected by filtration. The solid was dissolved in THF, diluted with ethyl acetate and most of the THF was removed under reduced pressure (by adding ethyl acetate several times and evaporation under reduced pressure) to give a thick suspension. The solid was collected by filtration, washed with ethyl acetate and dried to give 3-[[4-(2,6-dimethylphenyl)-6-[(2R)-2-(spiro[2.3]hexan-5-ylamino)-3-[1-(trifluoromethyl)cyclopropyl]propoxy]pyrimidin-2-yl]sulfamoyl]benzoic acid (hydrochloride salt) as an off-white solid (97 g, 98%). ESI-MS m/z calc. 644.228, found 645.0 (M+1)⁺; Retention time: 1.31 minutes (LC method A).

### Step 3: (11R)-6-(2,6-Dimethylphenyl)-2,2-dioxo-12-spiro[2.3]hexan-5-yl-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (Compound 198)

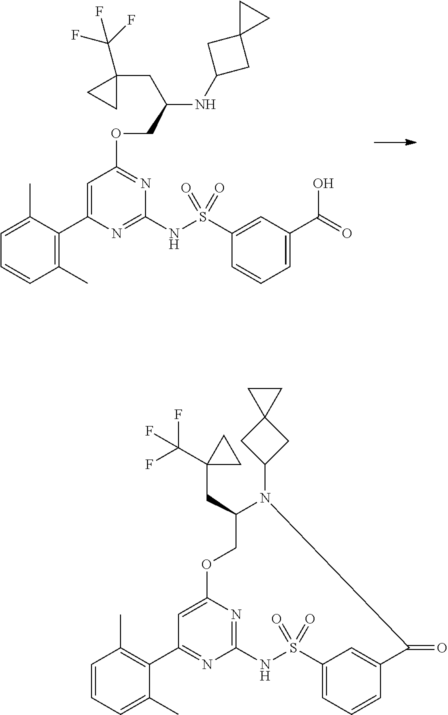 [SMILES: Cc1cccc(C)c1-c1cc(OC[C@@H](CC2(C(F)(F)F)CC2)NC2CC3(CC3)C2)nc(NS(=O)(=O)c2cccc(C(=O)O)c2)n1 ; Cc1cccc(C)c1-c1cc2nc(n1)NS(=O)(=O)c1cccc(c1)C(=O)N(C1CC3(CC3)C1)[C@H](CC1(C(F)(F)F)CC1)CO2]

3-[[4-(2,6-Dimethylphenyl)-6-[(2R)-2-(spiro[2.3]hexan-5-ylamino)-3-[1-(trifluoromethyl)cyclopropyl]propoxy]pyrimidin-2-yl]sulfamoyl]benzoic acid (hydrochloride salt) (96.9 g, 142.3 mmol) was dissolved in DMF (2.9 L) (clear colorless solution), purged with nitrogen (3×vacuum/N₂), treated with DIEA (124 mL, 711.9 mmol) first and then immediately with HATU (81.2 g, 213.6 mmol) and the reaction was stirred at room temperature for 19 h. Then another portion of HATU (5.4 g, 14.20 mmol) was added and the reaction was stirred at room temperature for another 2 h. The yellow orange solution was concentrated under reduced pressure at 50-55° C. to give a yellow orange mass/glass, which was treated with citric acid (710 mL of 1 M, 710.0 mmol) and ice water (700 mL) and the resulting mixture was stirred at room temperature for 2 h to give a yellow suspension. The solid was collected by filtration, washed with plenty of water and sucked dry overnight. The solid was dissolved in ethyl acetate (1 L) and MeTHF (1 L) under warming to give a yellow orange solution with a yellow aqueous phase and washed 3 times with 0.5 M HCl (600, 400 and 200 mL) and twice with brine (400 and 200 mL) and the aqueous phases were back extracted once with ethyl acetate (250 mL). The combined organic phases were dried, filtered and the clear yellow orange solution was evaporated and dried to give 120 g of a yellow orange foam. The foam was dissolved in methanol (1 L) and it was seeded with material from another reaction which resulted in slow crystallization. The orange suspension was stirred at room temperature for 0.5 h then slowly concentrated at 50-55° C. under reduced pressure to half of its original volume (˜500 mL). The orange suspension was stirred at room temperature for 2 h, the solid collected by filtration, washed with cold methanol (3 small portions, filtrate from orange to colorless) and dried at 45° C. under reduced pressure with a nitrogen leak overnight to give (11R)-6-(2,6-dimethylphenyl)-2,2-dioxo-12-spiro[2.3]hexan-5-yl-11-[[1-(trifluoromethyl)cyclopropyl]methyl]-9-oxa-2λ⁶-thia-3,5,12,19-tetrazatricyclo[12.3.1.14,8]nonadeca-1(18),4(19),5,7,14,16-hexaen-13-one (54 g, 60%) as an off-white solid. ¹H NMR (500 MHz, DMSO-d₆) δ 13.04 (s, 1H), 8.35 (s, 1H), 7.89 (s, 1H), 7.67 (d, J=22.8 Hz, 2H), 7.26 (s, 1H), 7.12 (s, 2H), 6.37 (s, 1H), 5.06 (dd, J=10.8, 4.5 Hz, 1H), 4.38 (t, J=11.5 Hz, 1H), 4.13 (ddd, J=24.3, 16.3, 10.4 Hz, 2H), 3.22 (t, J=9.4 Hz, 1H), 2.31-1.75 (m, 9H), 1.54 (dd, J=16.5, 9.3 Hz, 1H), 0.82 (dt, J=10.8, 5.4 Hz, 1H), 0.75 (dt, J=10.2, 5.2 Hz, 1H), 0.66 (d, J=6.4 Hz, 1H), 0.60-0.33 (in, 5H). ESI-MS m/z calc. 626.21747, found 627.0 (M+1)⁺; Retention time: 2.02 minutes (LC method A).

### VI. Characterization of New Compounds

The compounds in the following tables were prepared in a manner analogous to that described above using commercially available reagents and intermediates described herein.

TABLE 3
LCMS data of new compounds

| Compound | | LCMS | Calc. | | LCMS |
|---|---|---|---|---|---|
| number | Structure | Rt (min) | mass | M + 1 | Method |
| 199 | 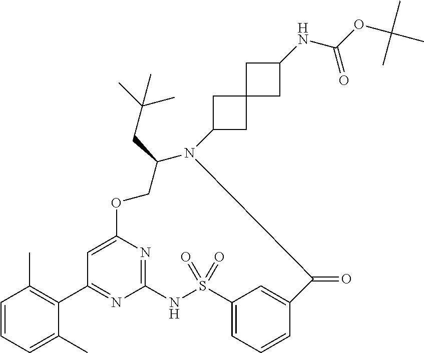 [SMILES: Cc1cccc(C)c1-c1cc2nc(n1)NS(=O)(=O)c1cccc(c1)C(=O)N(C1CC3(CC(NC(=O)OC(C)(C)C)C3)C1)[C@H](CC(C)(C)C)CO2] | 2.15 | 703.34 | 704.5 | A |
| 200 | 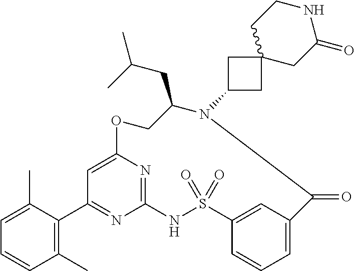 | 1.51 | 617.267 | 618.3 | A |
| 201 | 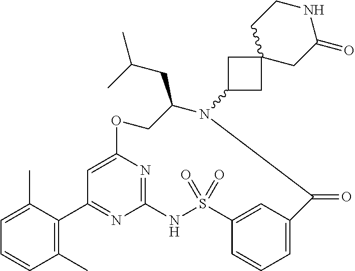 [SMILES: Cc1cccc(C)c1-c1cc2nc(n1)NS(=O)(=O)c1cccc(c1)C(=O)N(C1CC3(CCNC(=O)C3)C1)[C@H](CC(C)C)CO2] | 1.58 | 617.267 | 618.3 | A |
| 202 | 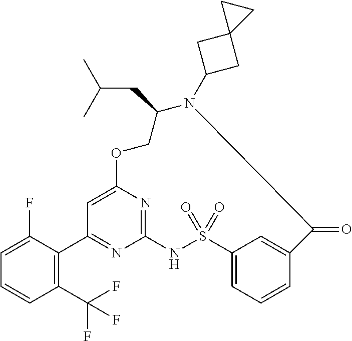 [SMILES: CC(C)C[C@@H]1COc2cc(-c3c(F)cccc3C(F)(F)F)nc(n2)NS(=O)(=O)c2cccc(c2)C(=O)N1C1CC2(CC2)C1] | 1.37 | 618.192 | 619 | A |
| 203 | 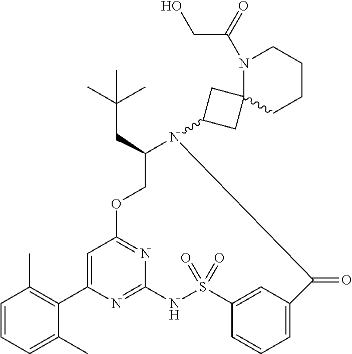 [SMILES: Cc1cccc(C)c1-c1cc2nc(n1)NS(=O)(=O)c1cccc(c1)C(=O)N(C1CC3(CCCCN3C(=O)CO)C1)[C@H](CC(C)(C)C)CO2] | 1.77 | 675.309 | 676.3 | A |
| 204 | 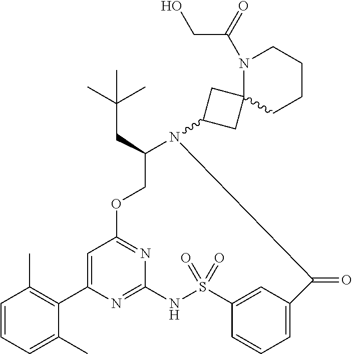 [SMILES: Cc1cccc(C)c1-c1cc2nc(n1)NS(=O)(=O)c1cccc(c1)C(=O)N(C1CC3(CCCCN3C(=O)CO)C1)[C@H](CC(C)(C)C)CO2] | 1.73 | 675.309 | 676.3 | A |
| 205 | 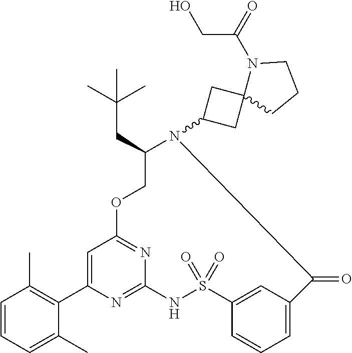 [SMILES: Cc1cccc(C)c1-c1cc2nc(n1)NS(=O)(=O)c1cccc(c1)C(=O)N(C1CC3(CCCN3C(=O)CO)C1)[C@H](CC(C)(C)C)CO2] | 1.75 | 661.293 | 662.2 | A |
| 206 | 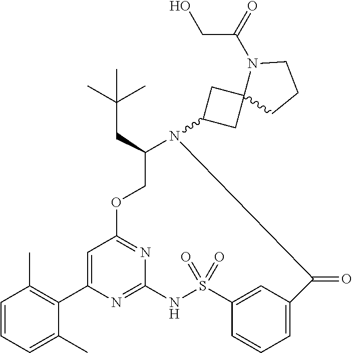 [SMILES: Cc1cccc(C)c1-c1cc2nc(n1)NS(=O)(=O)c1cccc(c1)C(=O)N(C1CC3(CCCN3C(=O)CO)C1)[C@H](CC(C)(C)C)CO2] | 1.67 | 661.293 | 662.3 | A |
| 207 | 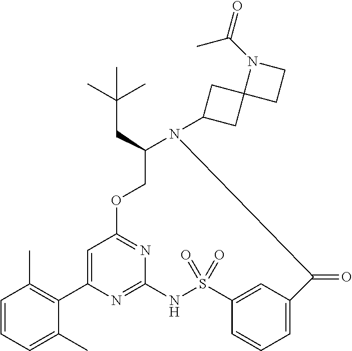 [SMILES: CC(=O)N1CCC12CC(N1C(=O)c3cccc(c3)S(=O)(=O)Nc3nc(cc(-c4c(C)cccc4C)n3)OC[C@H]1CC(C)(C)C)C2] | 1.65 | 631.283 | 632.4 | A |
| 208 | 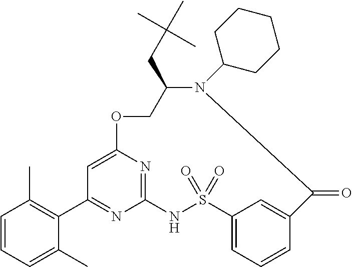 [SMILES: Cc1cccc(C)c1-c1cc2nc(n1)NS(=O)(=O)c1cccc(c1)C(=O)N(C1CCCCC1)[C@H](CC(C)(C)C)CO2] | 1.85 | 576.277 | 577.4 | A |
| 209 | 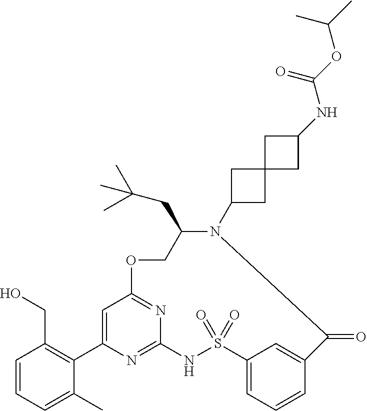 [SMILES: Cc1cccc(CO)c1-c1cc2nc(n1)NS(=O)(=O)c1cccc(c1)C(=O)N(C1CC3(CC(NC(=O)OC(C)C)C3)C1)[C@H](CC(C)(C)C)CO2] | 2.59 | 705.32 | 706.4 | T |
| 210 | 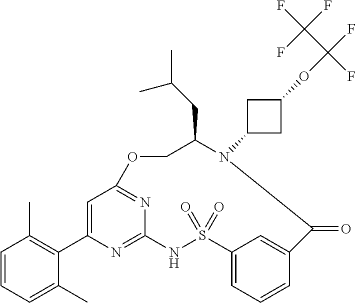 [SMILES: Cc1cccc(C)c1-c1cc2nc(n1)NS(=O)(=O)c1cccc(c1)C(=O)N([C@H]1C[C@@H](OC(F)(F)C(F)(F)F)C1)[C@H](CC(C)C)CO2] | 3.12 | 668.209 | 669.5 | T |
| 211 | 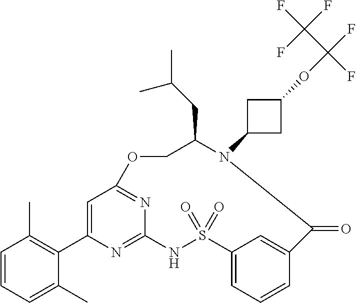 [SMILES: Cc1cccc(C)c1-c1cc2nc(n1)NS(=O)(=O)c1cccc(c1)C(=O)N([C@H]1C[C@H](OC(F)(F)C(F)(F)F)C1)[C@H](CC(C)C)CO2] | 3.23 | 668.209 | 669.2 | T |
| 212 | 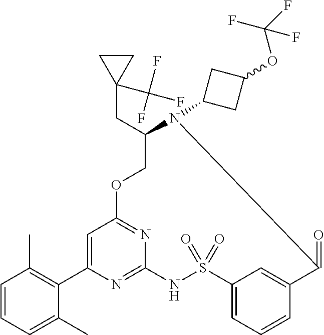 | 2.93 | 684.184 | 685.4 | W |
| 213 | 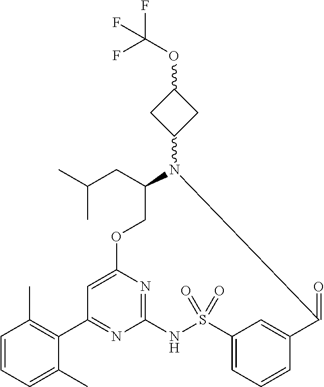 [SMILES: Cc1cccc(C)c1-c1cc2nc(n1)NS(=O)(=O)c1cccc(c1)C(=O)N(C1CC(OC(F)(F)F)C1)[C@H](CC(C)C)CO2] | 2.98 | 618.212 | 619.4 | W |
| 214 | 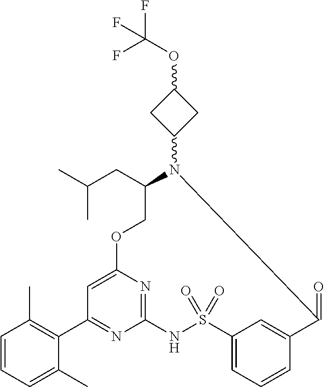 [SMILES: Cc1cccc(C)c1-c1cc2nc(n1)NS(=O)(=O)c1cccc(c1)C(=O)N(C1CC(OC(F)(F)F)C1)[C@H](CC(C)C)CO2] | 3.09 | 618.212 | 619.4 | W |
| 215 | 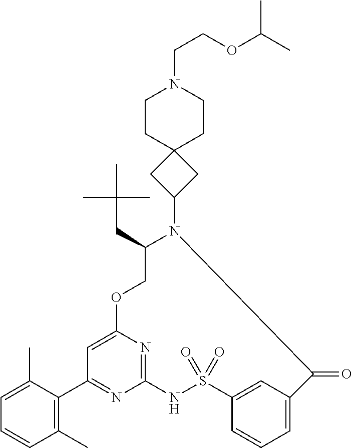 [SMILES: Cc1cccc(C)c1-c1cc2nc(n1)NS(=O)(=O)c1cccc(c1)C(=O)N(C1CC3(CCN(CCOC(C)C)CC3)C1)[C@H](CC(C)(C)C)CO2] | 1.52 | 703.377 | 704.8 | A |
| 216 | 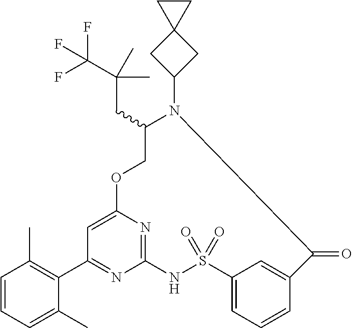 [SMILES: Cc1cccc(C)c1-c1cc2nc(n1)NS(=O)(=O)c1cccc(c1)C(=O)N(C1CC3(CC3)C1)C(CC(C)(C)C(F)(F)F)CO2] | 2.08 | 628.233 | 629.13 | A |
| 217 | 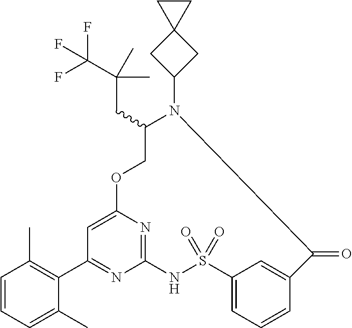 [SMILES: Cc1cccc(C)c1-c1cc2nc(n1)NS(=O)(=O)c1cccc(c1)C(=O)N(C1CC3(CC3)C1)C(CC(C)(C)C(F)(F)F)CO2] | 2.08 | 628.233 | 629.13 | A |
| 218 | 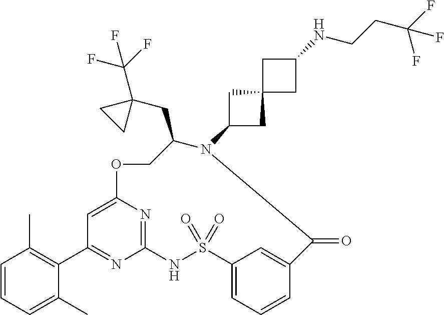 | 1.36 | 751.263 | 752.8 | A |
| 219 | 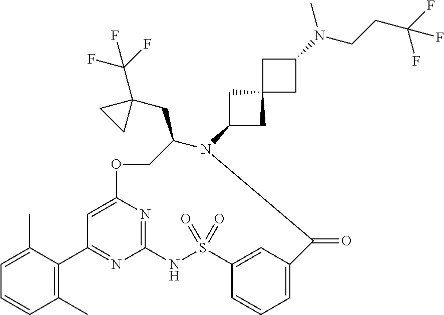 | 1.38 | 765.278 | 766.5 | A |
| 220 | 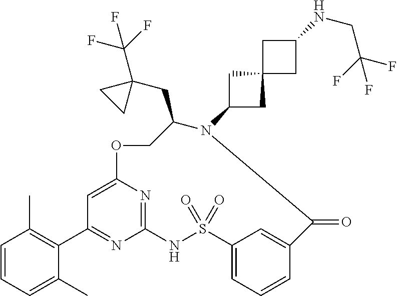 | 1.34 | 737.247 | 738.7 | A |
| 221 | 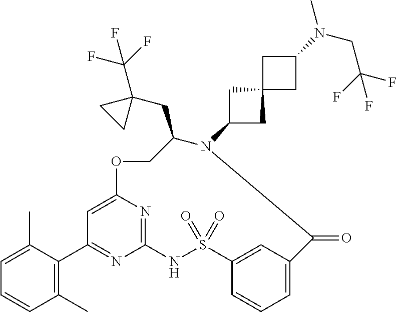 | 1.52 | 751.263 | 752.8 | A |
| 222 | 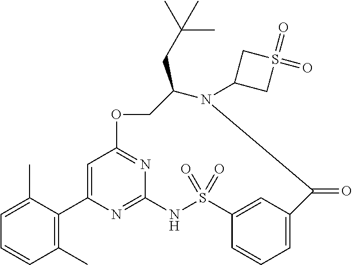 [SMILES: Cc1cccc(C)c1-c1cc2nc(n1)NS(=O)(=O)c1cccc(c1)C(=O)N(C1CS(=O)(=O)C1)[C@H](CC(C)(C)C)CO2] | 1.54 | 598.192 | 599.5 | A |
| 223 | 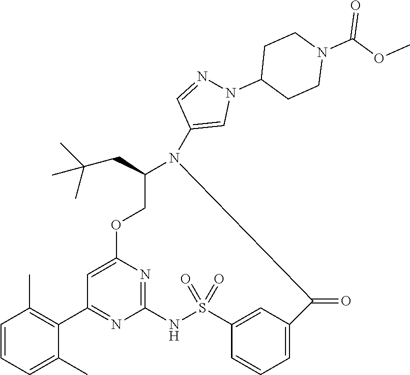 [SMILES: COC(=O)N1CCC(n2cc(N3C(=O)c4cccc(c4)S(=O)(=O)Nc4nc(cc(-c5c(C)cccc5C)n4)OC[C@H]3CC(C)(C)C)cn2)CC1] | 1.77 | 701.3 | 702.5 | A |
| 224 | 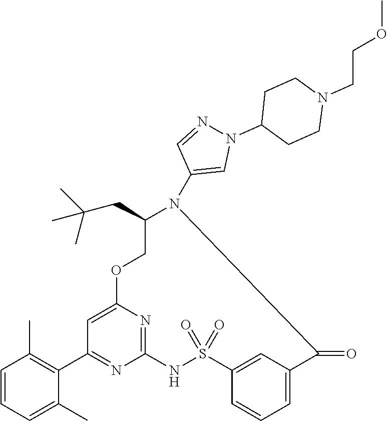 [SMILES: COCCN1CCC(n2cc(N3C(=O)c4cccc(c4)S(=O)(=O)Nc4nc(cc(-c5c(C)cccc5C)n4)OC[C@H]3CC(C)(C)C)cn2)CC1] | 1.53 | 701.336 | 702.6 | A |
| 6 | 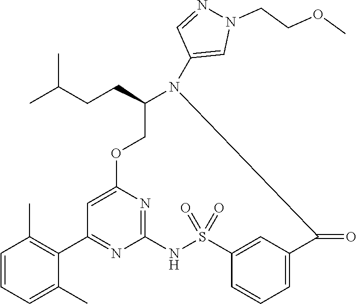 [SMILES: COCCn1cc(N2C(=O)c3cccc(c3)S(=O)(=O)Nc3nc(cc(-c4c(C)cccc4C)n3)OC[C@H]2CCC(C)C)cn1] | 1.65 | 618.262 | 619.5 | A |
| 225 | 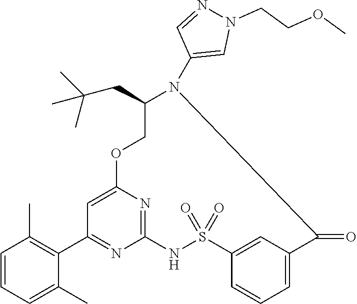 [SMILES: COCCn1cc(N2C(=O)c3cccc(c3)S(=O)(=O)Nc3nc(cc(-c4c(C)cccc4C)n3)OC[C@H]2CC(C)(C)C)cn1] | 1.6 | 618.262 | 619.6 | A |
| 226 | 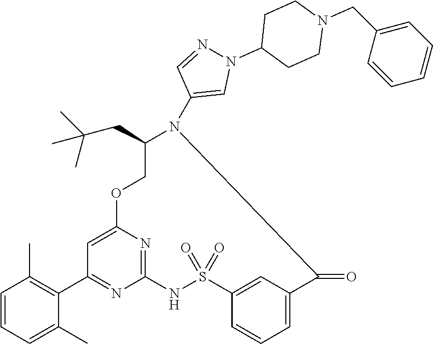 [SMILES: Cc1cccc(C)c1-c1cc2nc(n1)NS(=O)(=O)c1cccc(c1)C(=O)N(c1cnn(C3CCN(Cc4ccccc4)CC3)c1)[C@H](CC(C)(C)C)CO2] | 2.14 | 733.341 | 735.7 | A |
| 227 | 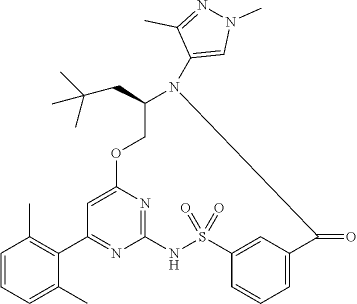 [SMILES: Cc1cccc(C)c1-c1cc2nc(n1)NS(=O)(=O)c1cccc(c1)C(=O)N(c1cn(C)nc1C)[C@H](CC(C)(C)C)CO2] | 1.57 | 588.252 | 589.5 | A |
| 7 | 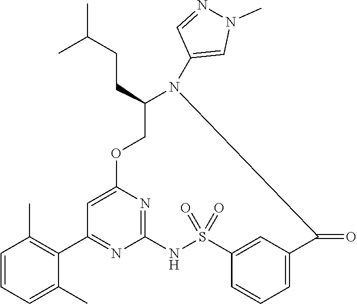 [SMILES: Cc1cccc(C)c1-c1cc2nc(n1)NS(=O)(=O)c1cccc(c1)C(=O)N(c1cnn(C)c1)[C@H](CCC(C)C)CO2] | 1.61 | 574.236 | 575.5 | A |
| 228 | 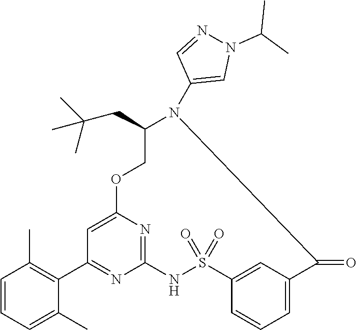 [SMILES: Cc1cccc(C)c1-c1cc2nc(n1)NS(=O)(=O)c1cccc(c1)C(=O)N(c1cnn(C(C)C)c1)[C@H](CC(C)(C)C)CO2] | 1.75 | 602.268 | 603.7 | A |
| 229 | 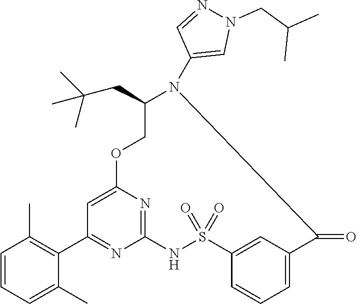 [SMILES: Cc1cccc(C)c1-c1cc2nc(n1)NS(=O)(=O)c1cccc(c1)C(=O)N(c1cnn(CC(C)C)c1)[C@H](CC(C)(C)C)CO2] | 1.86 | 616.283 | 617.6 | A |
| 230 | 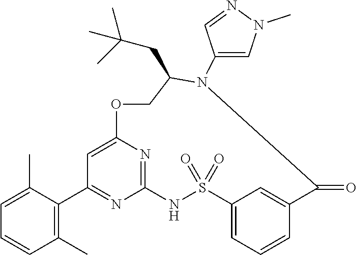 [SMILES: Cc1cccc(C)c1-c1cc2nc(n1)NS(=O)(=O)c1cccc(c1)C(=O)N(c1cnn(C)c1)[C@H](CC(C)(C)C)CO2] | 1.56 | 574.236 | 575.6 | A |
| 231 | 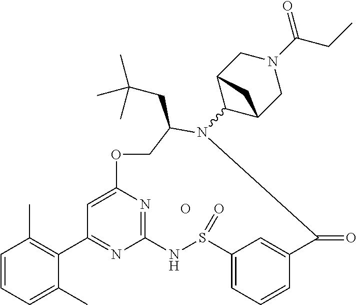 | 1.66 | 645.298 | 646.7 | A |
| 232 | 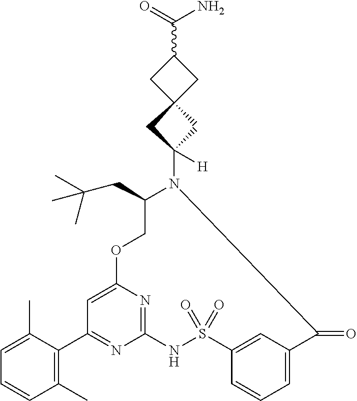 | 1.57 | 631.283 | 632.7 | A |
| 233 | 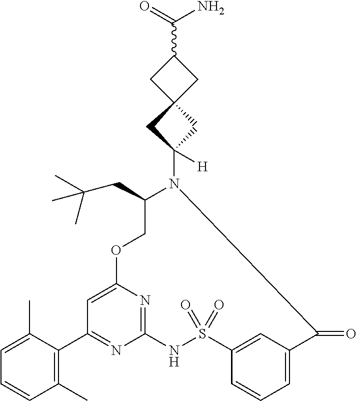 | 1.55 | 631.283 | 632.5 | A |
| 234 | 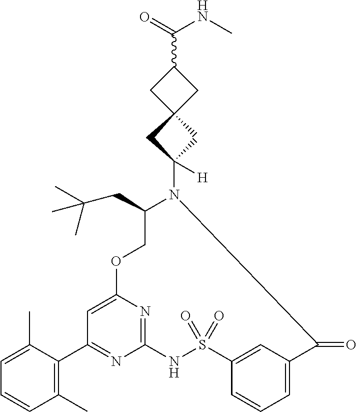 | 1.62 | 645.298 | 646.7 | A |
| 235 | 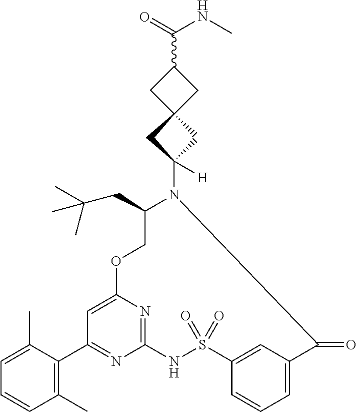 | 1.62 | 645.298 | 646.8 | A |
| 236 | 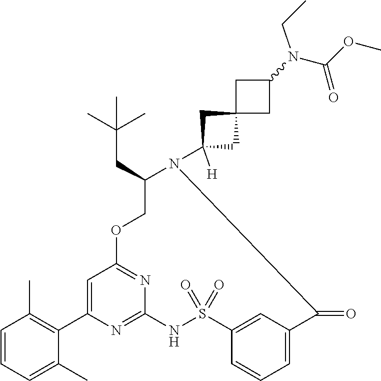 | 2.04 | 689.325 | 690.7 | A |
| 237 | 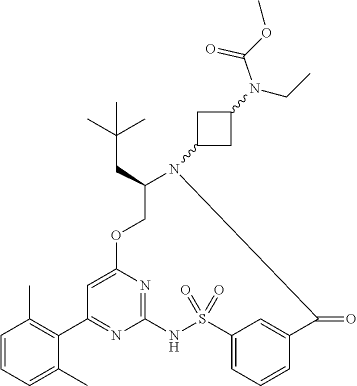 [SMILES: CCN(C(=O)OC)C1CC(N2C(=O)c3cccc(c3)S(=O)(=O)Nc3nc(cc(-c4c(C)cccc4C)n3)OC[C@H]2CC(C)(C)C)C1] | 1.9 | 649.293 | 650.7 | A |
| 238 | 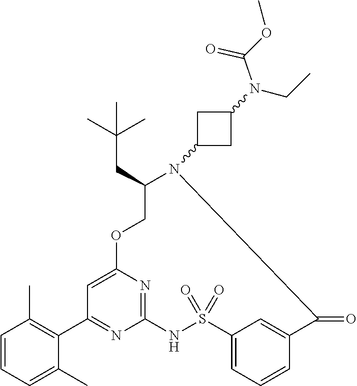 [SMILES: CCN(C(=O)OC)C1CC(N2C(=O)c3cccc(c3)S(=O)(=O)Nc3nc(cc(-c4c(C)cccc4C)n3)OC[C@H]2CC(C)(C)C)C1] | 1.91 | 649.293 | 650.7 | A |
| 239 | 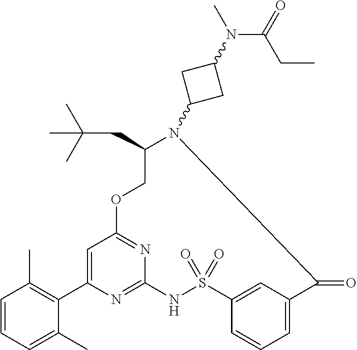 [SMILES: CCC(=O)N(C)C1CC(N2C(=O)c3cccc(c3)S(=O)(=O)Nc3nc(cc(-c4c(C)cccc4C)n3)OC[C@H]2CC(C)(C)C)C1] | 1.72 | 633.298 | 634.5 | A |
| 240 | 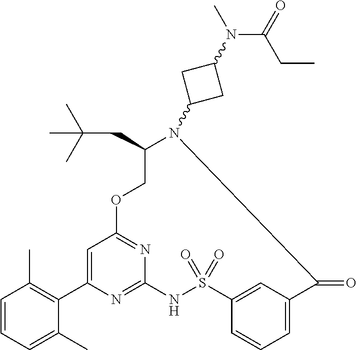 [SMILES: CCC(=O)N(C)C1CC(N2C(=O)c3cccc(c3)S(=O)(=O)Nc3nc(cc(-c4c(C)cccc4C)n3)OC[C@H]2CC(C)(C)C)C1] | 1.71 | 633.298 | 634.5 | A |
| 241 | 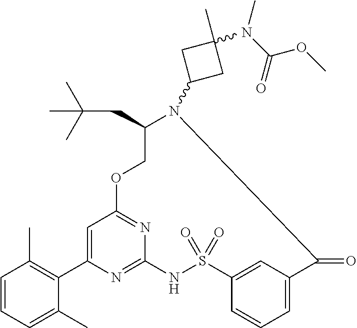 [SMILES: COC(=O)N(C)C1(C)CC(N2C(=O)c3cccc(c3)S(=O)(=O)Nc3nc(cc(-c4c(C)cccc4C)n3)OC[C@H]2CC(C)(C)C)C1] | 1.87 | 649.293 | 650.6 | A |
| 242 | 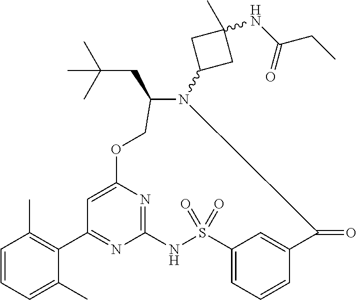 [SMILES: CCC(=O)NC1(C)CC(N2C(=O)c3cccc(c3)S(=O)(=O)Nc3nc(cc(-c4c(C)cccc4C)n3)OC[C@H]2CC(C)(C)C)C1] | 1.67 | 633.298 | 634.7 | A |
| 243 | 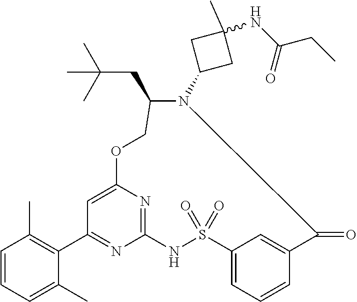 | 1.66 | 633.298 | 634.5 | A |
| 244 | 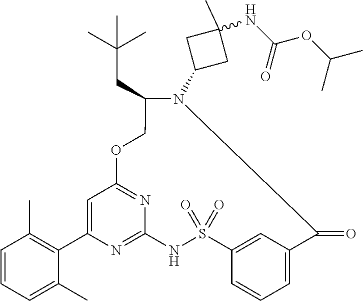 | 1.95 | 663.309 | 664.7 | A |
| 245 | 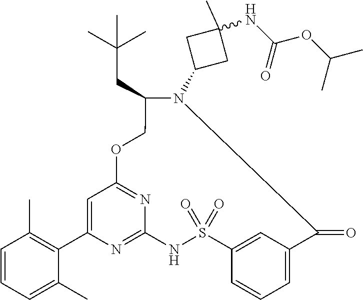 | 1.93 | 663.309 | 664.7 | A |
| 246 | 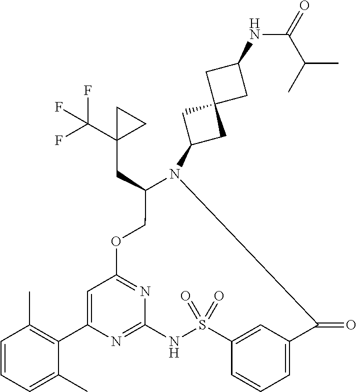 | 1.73 | 725.286 | 726.7 | A |
| 247 | 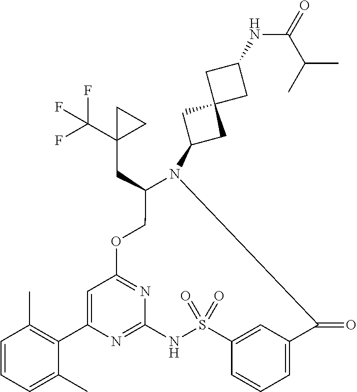 | 1.71 | 725.286 | 726.6 | A |
| 175 | 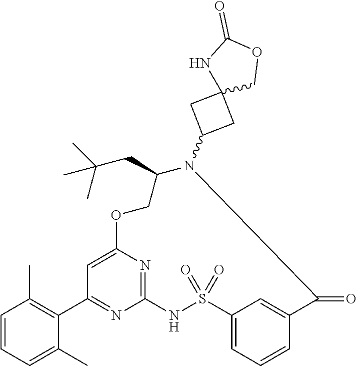 [SMILES: Cc1cccc(C)c1-c1cc2nc(n1)NS(=O)(=O)c1cccc(c1)C(=O)N(C1CC3(COC(=O)N3)C1)[C@H](CC(C)(C)C)CO2] | 1.51 | 619.246 | 620.6 | A |
| 176 | 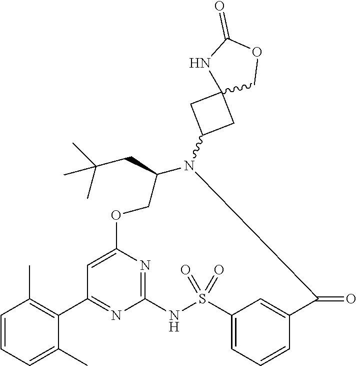 [SMILES: Cc1cccc(C)c1-c1cc2nc(n1)NS(=O)(=O)c1cccc(c1)C(=O)N(C1CC3(COC(=O)N3)C1)[C@H](CC(C)(C)C)CO2] | 1.59 | 619.246 | 620.5 | A |
| 248 | 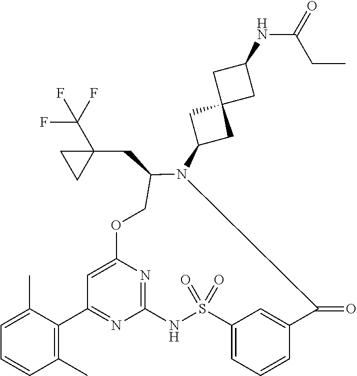 | 1.64 | 711.27 | 712.5 | A |
| 249 | 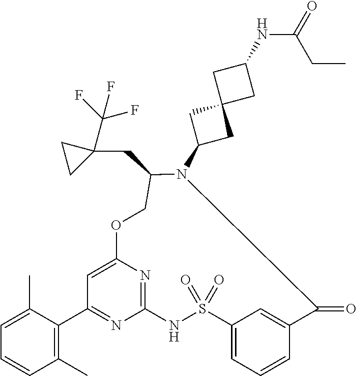 | 1.63 | 711.27 | 712.5 | A |
| 250 | 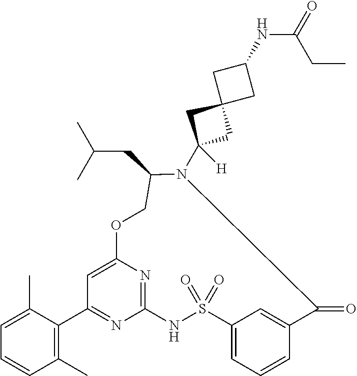 | 1.66 | 645.298 | 646.6 | A |
| 251 | 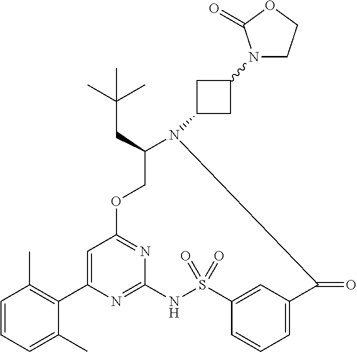 | 1.6 | 633.262 | 634.6 | A |
| 252 | 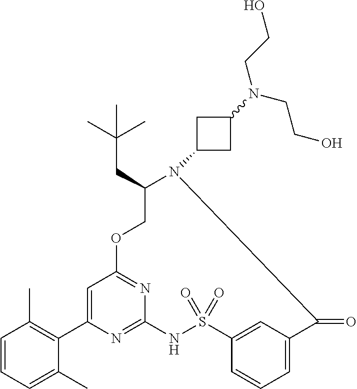 | 1.16 | 651.309 | 652.7 | A |
| 253 | 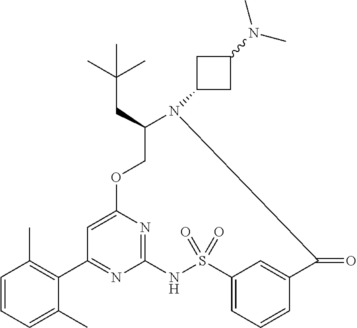 | 1.24 | 591.288 | 592.4 | A |
| 254 | 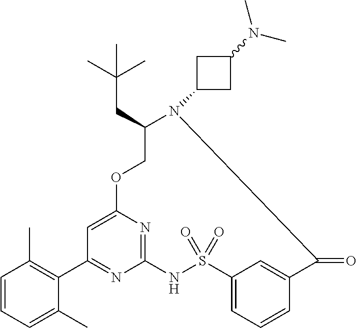 | 1.2 | 591.288 | 592.7 | A |
| 255 | 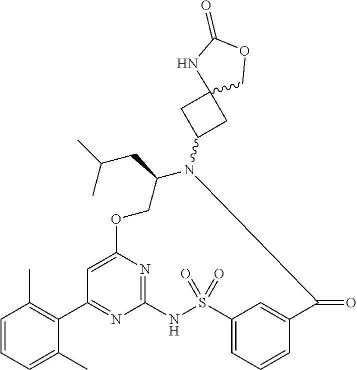 [SMILES: Cc1cccc(C)c1-c1cc2nc(n1)NS(=O)(=O)c1cccc(c1)C(=O)N(C1CC3(COC(=O)N3)C1)[C@H](CC(C)C)CO2] | 1.46 | 605.231 | 606.4 | A |
| 256 | 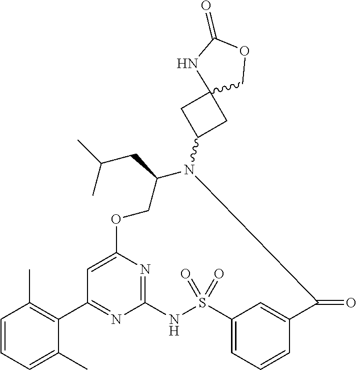 [SMILES: Cc1cccc(C)c1-c1cc2nc(n1)NS(=O)(=O)c1cccc(c1)C(=O)N(C1CC3(COC(=O)N3)C1)[C@H](CC(C)C)CO2] | 1.54 | 605.231 | 606.4 | A |
| 257 | 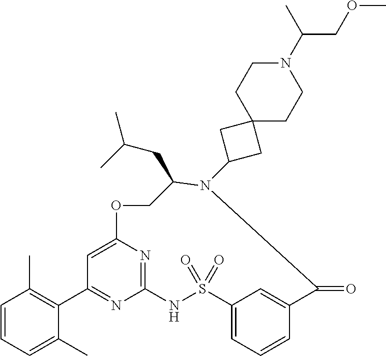 [SMILES: COCC(C)N1CCC2(CC1)CC(N1C(=O)c3cccc(c3)S(=O)(=O)Nc3nc(cc(-c4c(C)cccc4C)n3)OC[C@H]1CC(C)C)C2] | 1.4 | 675.345 | 676.7 | A |
| 258 | 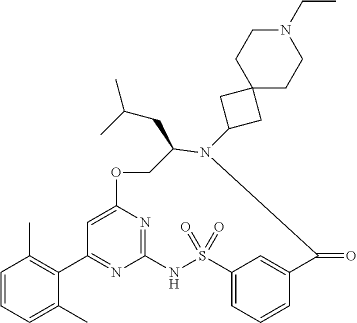 [SMILES: CCN1CCC2(CC1)CC(N1C(=O)c3cccc(c3)S(=O)(=O)Nc3nc(cc(-c4c(C)cccc4C)n3)OC[C@H]1CC(C)C)C2] | 1.34 | 631.319 | 632.7 | A |
| 259 | 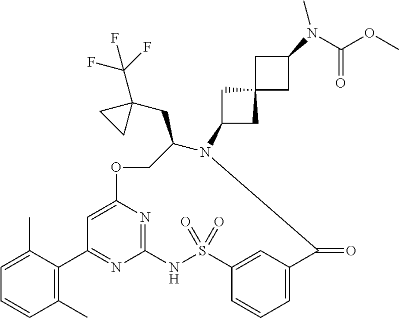 | 1.86 | 727.265 | 728.6 | A |
| 260 | 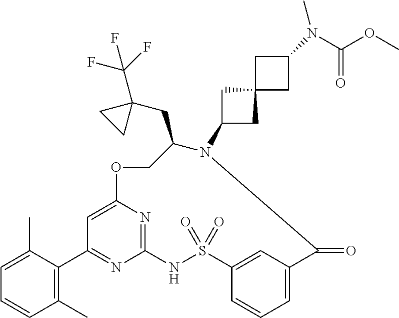 | 1.85 | 727.265 | 728.5 | A |
| 261 | 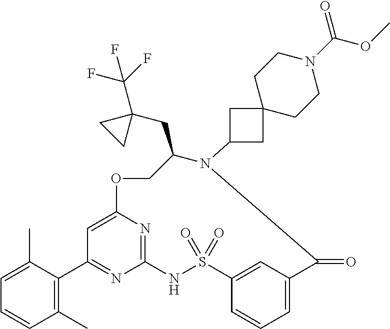 [SMILES: COC(=O)N1CCC2(CC1)CC(N1C(=O)c3cccc(c3)S(=O)(=O)Nc3nc(cc(-c4c(C)cccc4C)n3)OC[C@H]1CC1(C(F)(F)F)CC1)C2] | 1.84 | 727.265 | 728.6 | A |
| 262 | 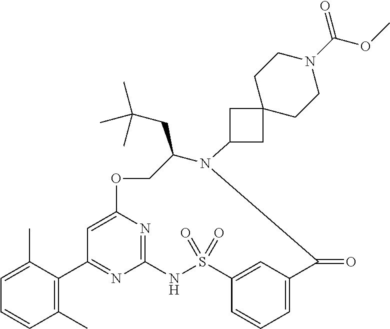 [SMILES: COC(=O)N1CCC2(CC1)CC(N1C(=O)c3cccc(c3)S(=O)(=O)Nc3nc(cc(-c4c(C)cccc4C)n3)OC[C@H]1CC(C)(C)C)C2] | 1.95 | 675.309 | 676.6 | A |
| 263 | 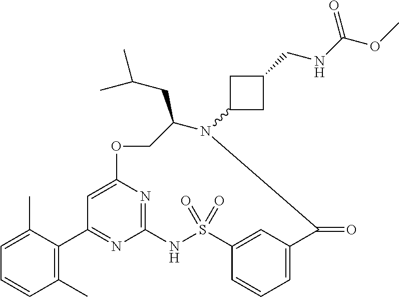 | 1.73 | 621.262 | 622.8 | A |
| 264 | 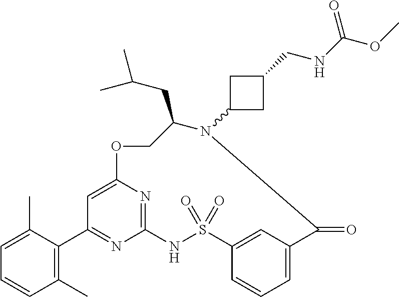 | 1.67 | 621.262 | 622.5 | A |
| 265 | 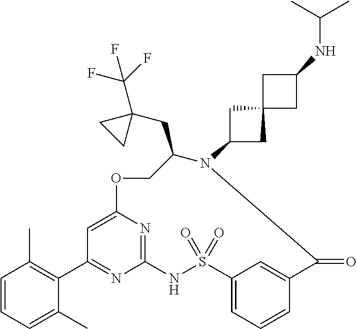 | 1.32 | 697.291 | 698.7 | A |
| 266 | 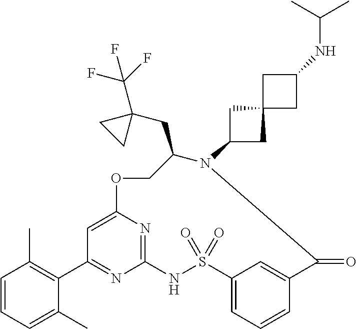 | 1.35 | 697.291 | 698.7 | A |
| 267 | 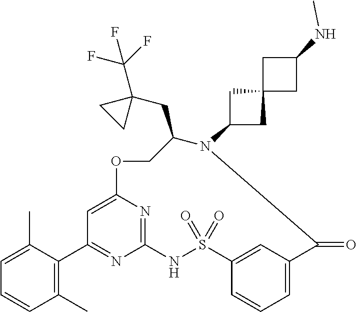 | 1.26 | 669.26 | 670.7 | A |
| 268 | 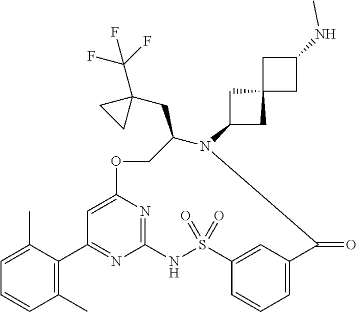 | 1.27 | 669.26 | 670.8 | A |
| 269 | 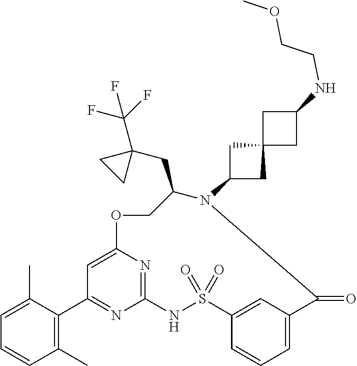 | 1.3 | 713.286 | 714.7 | A |
| 270 | 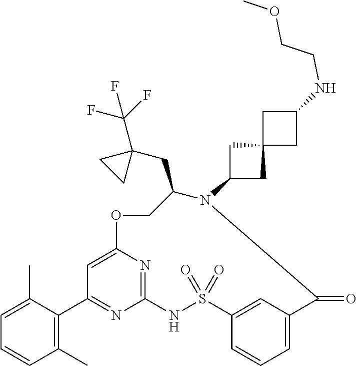 | 1.32 | 713.286 | 714.6 | A |
| 271 | 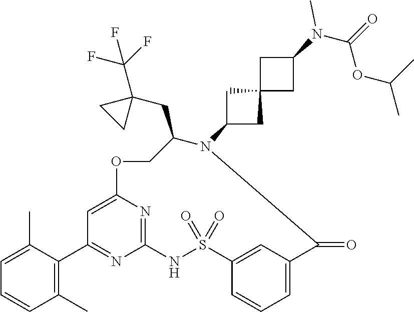 | 2.03 | 755.296 | 756.8 | A |
| 272 | 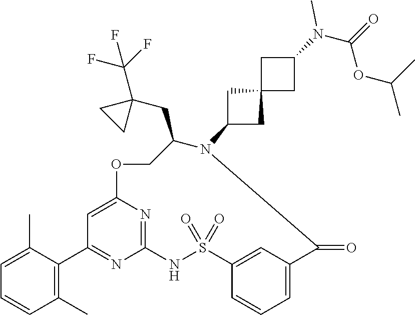 | 2.03 | 755.296 | 756.8 | A |
| 273 | 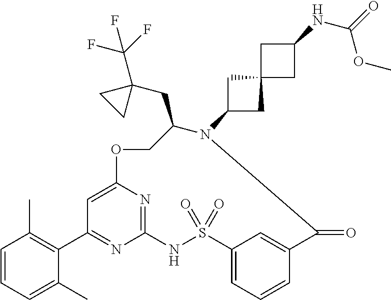 | 1.72 | 713.249 | 714.5 | A |
| 274 | 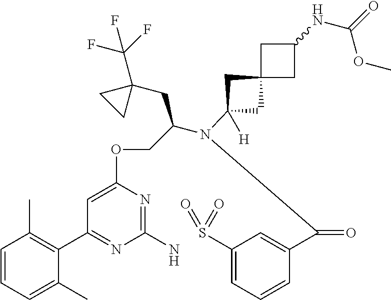 | 1.72 | 713.249 | 714.6 | A |
| 275 | 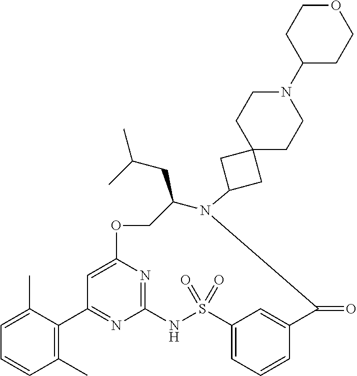 [SMILES: Cc1cccc(C)c1-c1cc2nc(n1)NS(=O)(=O)c1cccc(c1)C(=O)N(C1CC3(CCN(C4CCOCC4)CC3)C1)[C@H](CC(C)C)CO2] | 1.31 | 687.345 | 688.8 | A |
| 276 | 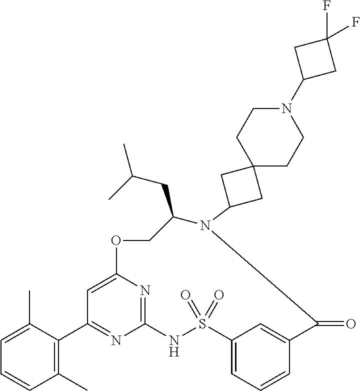 [SMILES: Cc1cccc(C)c1-c1cc2nc(n1)NS(=O)(=O)c1cccc(c1)C(=O)N(C1CC3(CCN(C4CC(F)(F)C4)CC3)C1)[C@H](CC(C)C)CO2] | 1.38 | 693.316 | 694.7 | A |
| 277 | 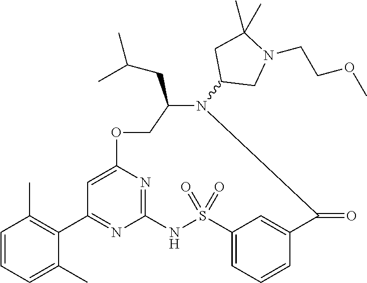 [SMILES: COCCN1CC(N2C(=O)c3cccc(c3)S(=O)(=O)Nc3nc(cc(-c4c(C)cccc4C)n3)OC[C@H]2CC(C)C)CC1(C)C] | 1.27 | 635.314 | 636.6 | A |
| 278 | 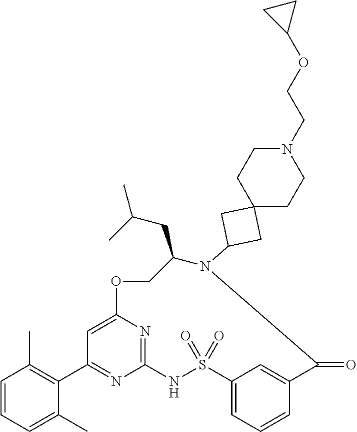 [SMILES: Cc1cccc(C)c1-c1cc2nc(n1)NS(=O)(=O)c1cccc(c1)C(=O)N(C1CC3(CCN(CCOC4CC4)CC3)C1)[C@H](CC(C)C)CO2] | 1.43 | 687.345 | 688.5 | A |
| 279 | 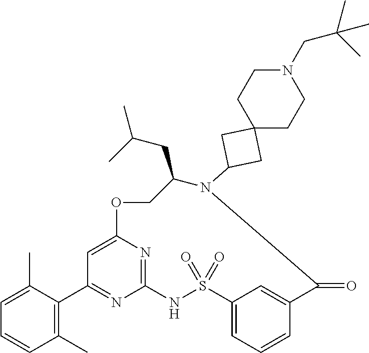 [SMILES: Cc1cccc(C)c1-c1cc2nc(n1)NS(=O)(=O)c1cccc(c1)C(=O)N(C1CC3(CCN(CC(C)(C)C)CC3)C1)[C@H](CC(C)C)CO2] | 1.46 | 673.366 | 674.6 | A |
| 280 | 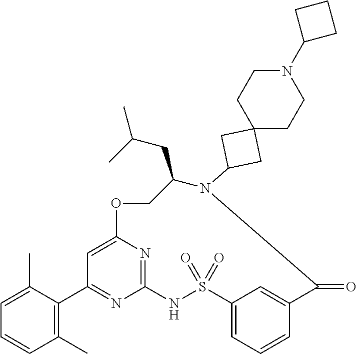 [SMILES: Cc1cccc(C)c1-c1cc2nc(n1)NS(=O)(=O)c1cccc(c1)C(=O)N(C1CC3(CCN(C4CCC4)CC3)C1)[C@H](CC(C)C)CO2] | 1.38 | 657.335 | 658.7 | A |
| 281 | 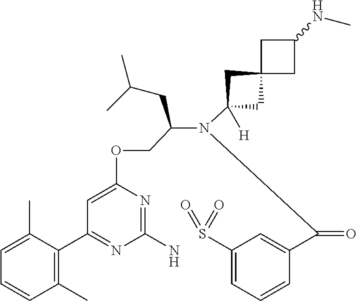 | 1.27 | 603.288 | 604.6 | A |
| 282 | 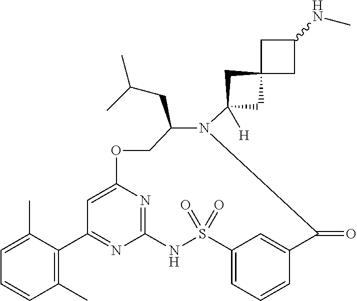 | 1.29 | 603.288 | 604.6 | A |
| 283 | 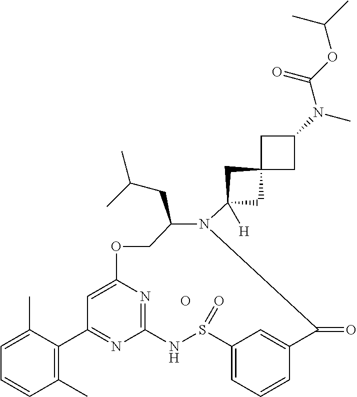 | 2.12 | 689.325 | 690.7 | A |
| 284 | 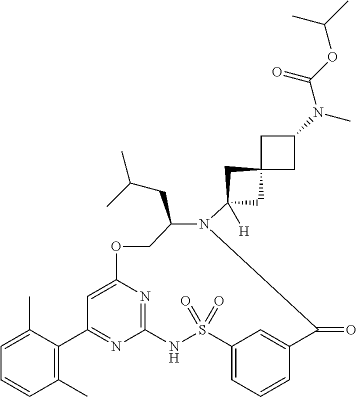 | 2.1 | 689.325 | 690.5 | A |
| 285 | 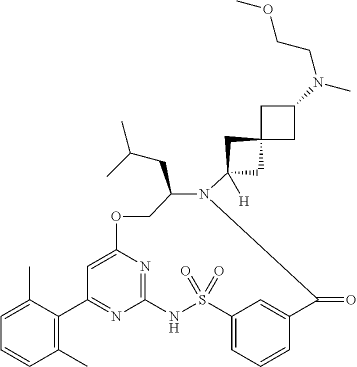 | 1.34 | 661.33 | 662.7 | A |
| 286 | 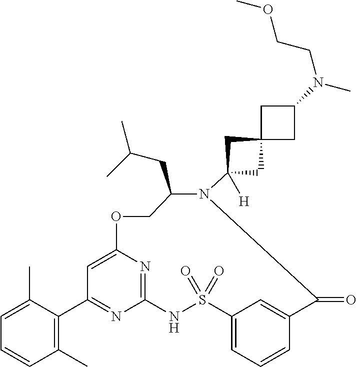 | 1.36 | 661.33 | 662.5 | A |
| 287 | 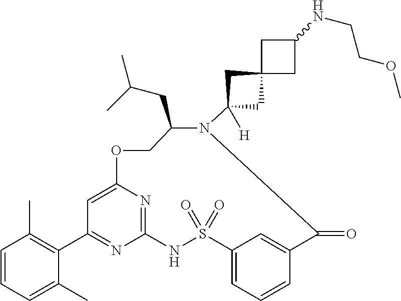 | 1.32 | 647.314 | 648.7 | A |
| 288 | 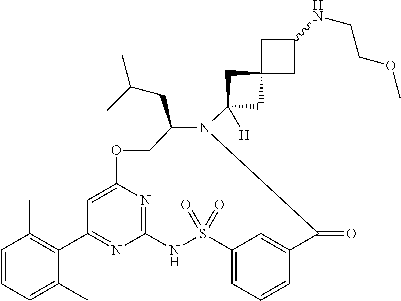 | 1.34 | 647.314 | 648.7 | A |
| 289 | 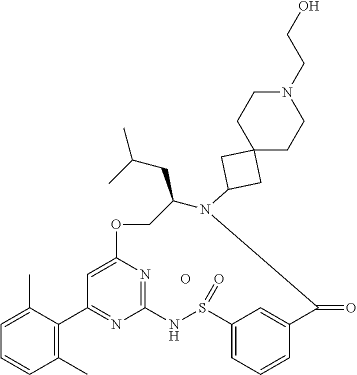 | 1.27 | 647.314 | 648.5 | A |
| 290 | 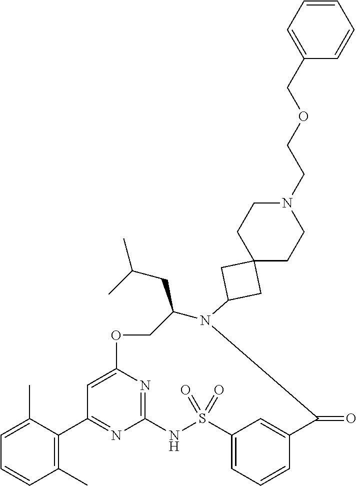 [SMILES: Cc1cccc(C)c1-c1cc2nc(n1)NS(=O)(=O)c1cccc(c1)C(=O)N(C1CC3(CCN(CCOCc4ccccc4)CC3)C1)[C@H](CC(C)C)CO2] | 1.57 | 737.361 | 738.5 | A |
| 291 | 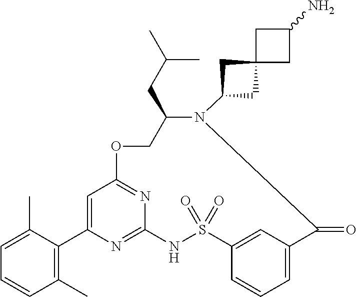 | 1.25 | 589.272 | 590.6 | A |
| 292 | 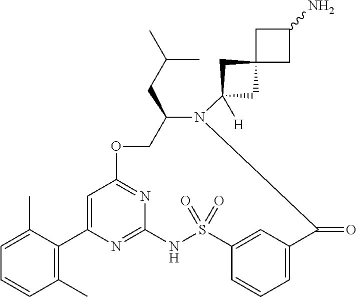 | 1.26 | 589.272 | 590.7 | A |
| 293 | 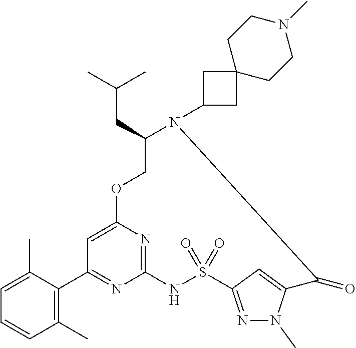 [SMILES: Cc1cccc(C)c1-c1cc2nc(n1)NS(=O)(=O)c1cc(n(C)n1)C(=O)N(C1CC3(CCN(C)CC3)C1)[C@H](CC(C)C)CO2] | 1.17 | 621.31 | 622.7 | A |
| 294 | 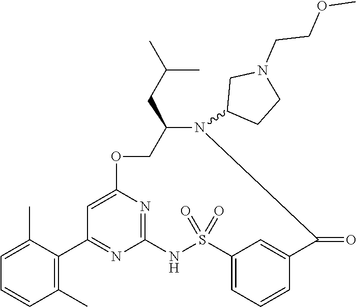 [SMILES: COCCN1CCC(N2C(=O)c3cccc(c3)S(=O)(=O)Nc3nc(cc(-c4c(C)cccc4C)n3)OC[C@H]2CC(C)C)C1] | 1.25 | 607.283 | 608.4 | A |

TABLE 4
LCMS data of new compounds

| Compound | | LCMS | Calc. | | LCMS |
|---|---|---|---|---|---|
| number | Structure | Rt (min) | mass | M + 1 | Method |
| 295 | 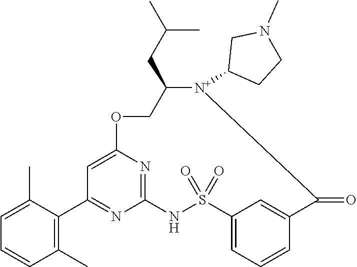 [SMILES: Cc1cccc(C)c1-c1cc2nc(n1)NS(=O)(=O)c1cccc(c1)C(=O)[NH+]([C@H]1CCN(C)C1)[C@H](CC(C)C)CO2] | 1.22 | 563.257 | 564.4 | A |
| 296 | 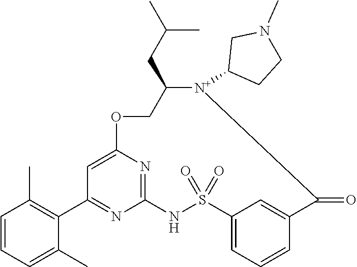 [SMILES: Cc1cccc(C)c1-c1cc2nc(n1)NS(=O)(=O)c1cccc(c1)C(=O)[NH+]([C@H]1CCN(C)C1)[C@H](CC(C)C)CO2] | 1.18 | 563.257 | 564.4 | A |
| 297 | 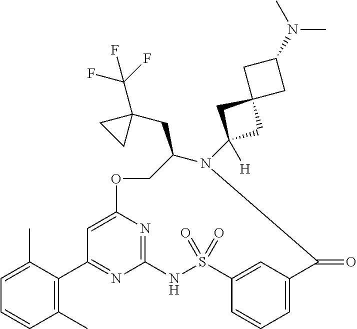 | 1.31 | 683.275 | 684.7 | A |
| 298 | 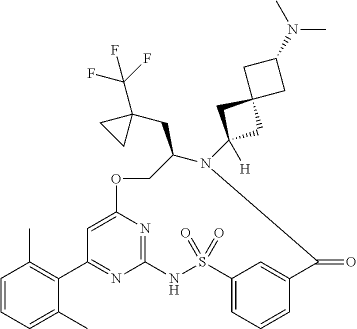 | 1.33 | 683.275 | 684.7 | A |
| 299 | 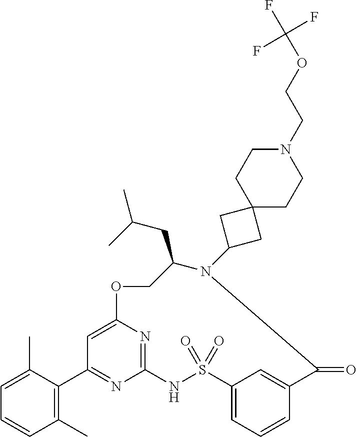 [SMILES: Cc1cccc(C)c1-c1cc2nc(n1)NS(=O)(=O)c1cccc(c1)C(=O)N(C1CC3(CCN(CCOC(F)(F)F)CC3)C1)[C@H](CC(C)C)CO2] | 1.47 | 715.302 | 716.7 | A |
| 300 | 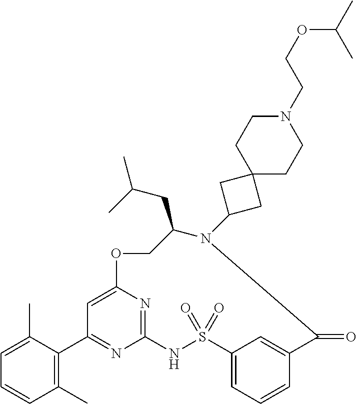 [SMILES: Cc1cccc(C)c1-c1cc2nc(n1)NS(=O)(=O)c1cccc(c1)C(=O)N(C1CC3(CCN(CCOC(C)C)CC3)C1)[C@H](CC(C)C)CO2] | 1.48 | 689.361 | 690.7 | A |
| 301 | 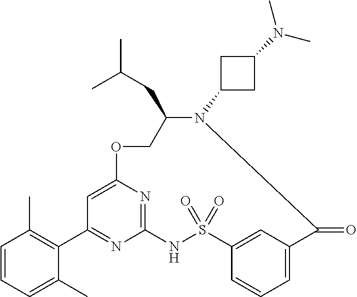 [SMILES: Cc1cccc(C)c1-c1cc2nc(n1)NS(=O)(=O)c1cccc(c1)C(=O)N([C@H]1C[C@@H](N(C)C)C1)[C@H](CC(C)C)CO2] | 1.23 | 577.272 | 578.4 | A |
| 302 | 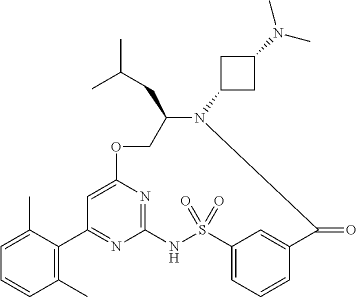 [SMILES: Cc1cccc(C)c1-c1cc2nc(n1)NS(=O)(=O)c1cccc(c1)C(=O)N([C@H]1C[C@@H](N(C)C)C1)[C@H](CC(C)C)CO2] | 1.18 | 577.272 | 578.6 | A |
| 303 | 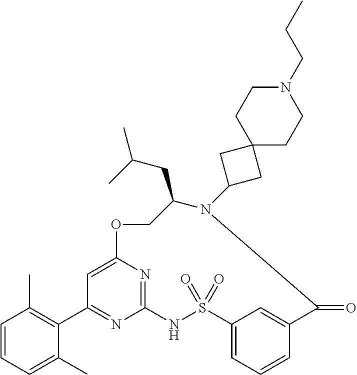 [SMILES: CCCN1CCC2(CC1)CC(N1C(=O)c3cccc(c3)S(=O)(=O)Nc3nc(cc(-c4c(C)cccc4C)n3)OC[C@H]1CC(C)C)C2] | 1.38 | 645.335 | 646.6 | A |
| 304 | 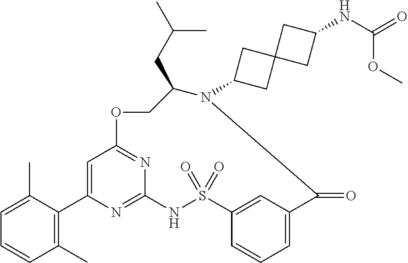 [SMILES: COC(=O)N[C@H]1CC2(C1)C[C@H](N1C(=O)c3cccc(c3)S(=O)(=O)Nc3nc(cc(-c4c(C)cccc4C)n3)OC[C@H]1CC(C)C)C2] | 1.76 | 647.278 | 648.4 | A |
| 305 | 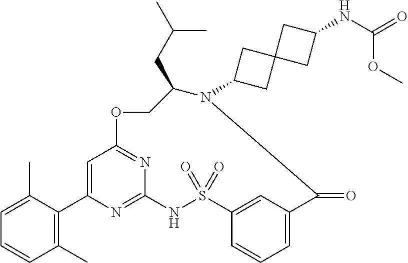 [SMILES: COC(=O)N[C@H]1CC2(C1)C[C@H](N1C(=O)c3cccc(c3)S(=O)(=O)Nc3nc(cc(-c4c(C)cccc4C)n3)OC[C@H]1CC(C)C)C2] | 1.75 | 647.278 | 648.3 | A |
| 306 | 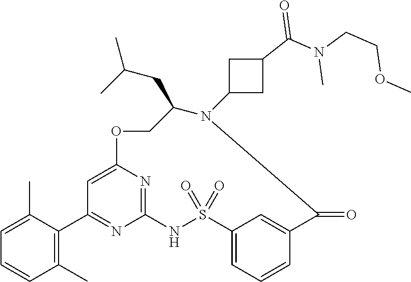 [SMILES: COCCN(C)C(=O)C1CC(N2C(=O)c3cccc(c3)S(=O)(=O)Nc3nc(cc(-c4c(C)cccc4C)n3)OC[C@H]2CC(C)C)C1] | 1.63 | 649.293 | 650.4 | A |
| 307 | 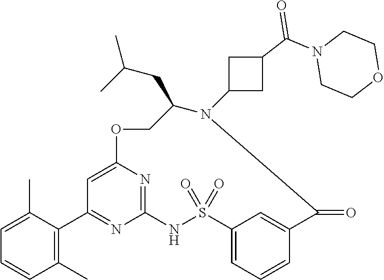 [SMILES: Cc1cccc(C)c1-c1cc2nc(n1)NS(=O)(=O)c1cccc(c1)C(=O)N(C1CC(C(=O)N3CCOCC3)C1)[C@H](CC(C)C)CO2] | 1.56 | 647.278 | 648.4 | A |
| 308 | 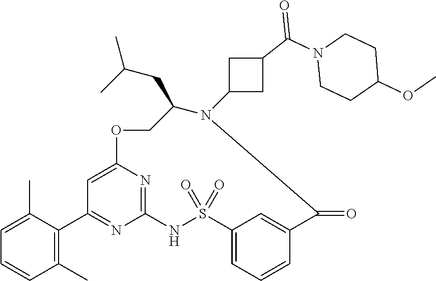 [SMILES: COC1CCN(C(=O)C2CC(N3C(=O)c4cccc(c4)S(=O)(=O)Nc4nc(cc(-c5c(C)cccc5C)n4)OC[C@H]3CC(C)C)C2)CC1] | 1.66 | 675.309 | 676.5 | A |
| 309 | 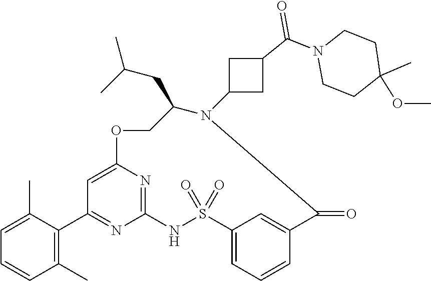 [SMILES: COC1(C)CCN(C(=O)C2CC(N3C(=O)c4cccc(c4)S(=O)(=O)Nc4nc(cc(-c5c(C)cccc5C)n4)OC[C@H]3CC(C)C)C2)CC1] | 1.75 | 689.325 | 690.5 | A |
| 310 | 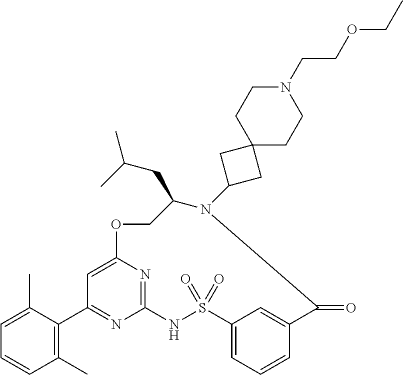 [SMILES: CCOCCN1CCC2(CC1)CC(N1C(=O)c3cccc(c3)S(=O)(=O)Nc3nc(cc(-c4c(C)cccc4C)n3)OC[C@H]1CC(C)C)C2] | 1.42 | 675.345 | 676.6 | A |
| 311 | 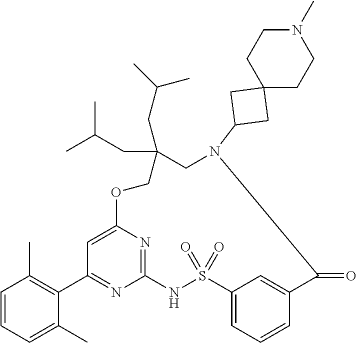 [SMILES: Cc1cccc(C)c1-c1cc2nc(n1)NS(=O)(=O)c1cccc(c1)C(=O)N(C1CC3(CCN(C)CC3)C1)CC(CC(C)C)(CC(C)C)CO2] | 1.62 | 687.382 | 688.8 | A |
| 312 | 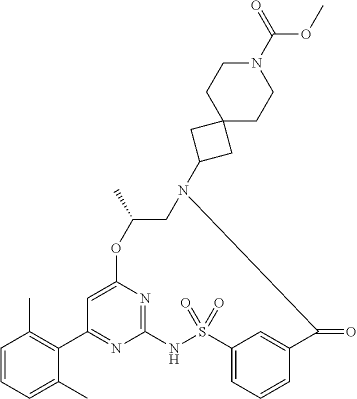 [SMILES: COC(=O)N1CCC2(CC1)CC(N1C[C@@H](C)Oc3cc(-c4c(C)cccc4C)nc(n3)NS(=O)(=O)c3cccc(c3)C1=O)C2] | 1.61 | 619.246 | 620.3 | A |
| 313 | 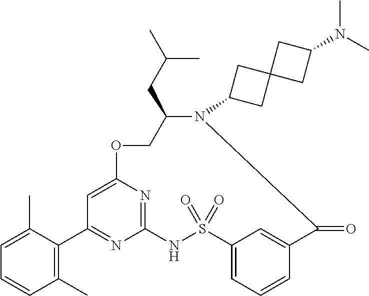 [SMILES: Cc1cccc(C)c1-c1cc2nc(n1)NS(=O)(=O)c1cccc(c1)C(=O)N([C@H]1CC3(C[C@H](N(C)C)C3)C1)[C@H](CC(C)C)CO2] | 1.325 | 617.304 | 618.4 | A |
| 314 | 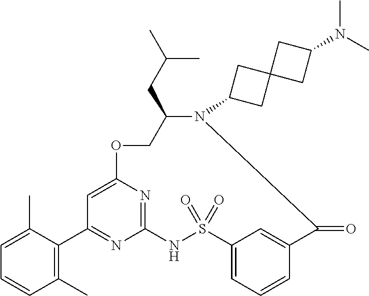 [SMILES: Cc1cccc(C)c1-c1cc2nc(n1)NS(=O)(=O)c1cccc(c1)C(=O)N([C@H]1CC3(C[C@H](N(C)C)C3)C1)[C@H](CC(C)C)CO2] | 1.35 | 617.304 | 618.4 | A |
| 315 | 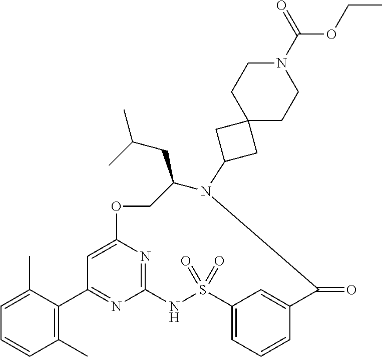 [SMILES: CCOC(=O)N1CCC2(CC1)CC(N1C(=O)c3cccc(c3)S(=O)(=O)Nc3nc(cc(-c4c(C)cccc4C)n3)OC[C@H]1CC(C)C)C2] | 1.93 | 675.309 | 676.4 | A |
| 316 | 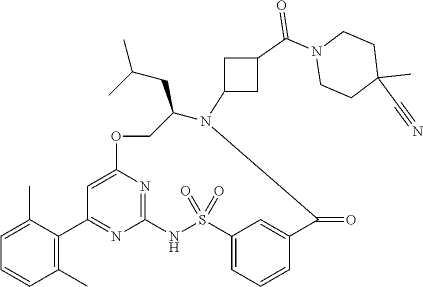 [SMILES: Cc1cccc(C)c1-c1cc2nc(n1)NS(=O)(=O)c1cccc(c1)C(=O)N(C1CC(C(=O)N3CCC(C)(C#N)CC3)C1)[C@H](CC(C)C)CO2] | 1.67 | 684.309 | 685.4 | A |
| 317 | 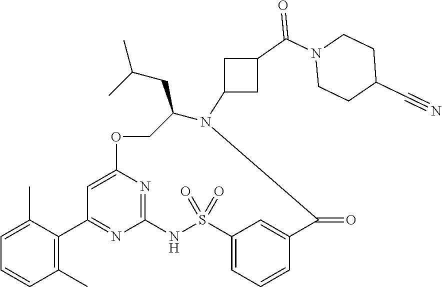 [SMILES: Cc1cccc(C)c1-c1cc2nc(n1)NS(=O)(=O)c1cccc(c1)C(=O)N(C1CC(C(=O)N3CCC(C#N)CC3)C1)[C@H](CC(C)C)CO2] | 1.62 | 670.294 | 671.5 | A |
| 318 | 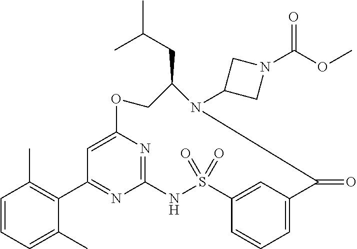 [SMILES: COC(=O)N1CC(N2C(=O)c3cccc(c3)S(=O)(=O)Nc3nc(cc(-c4c(C)cccc4C)n3)OC[C@H]2CC(C)C)C1] | 1.98 | 593.231 | 594.4 | A |
| 319 | 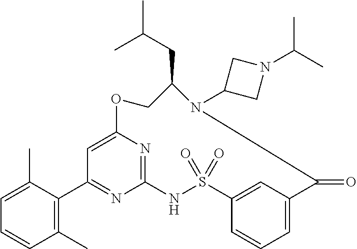 [SMILES: Cc1cccc(C)c1-c1cc2nc(n1)NS(=O)(=O)c1cccc(c1)C(=O)N(C1CN(C(C)C)C1)[C@H](CC(C)C)CO2] | 1.18 | 577.272 | 578.3 | A |
| 320 | 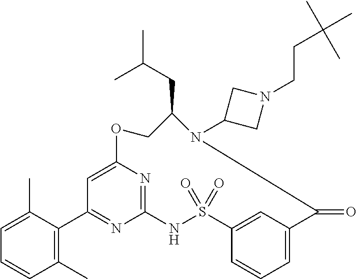 [SMILES: Cc1cccc(C)c1-c1cc2nc(n1)NS(=O)(=O)c1cccc(c1)C(=O)N(C1CN(CCC(C)(C)C)C1)[C@H](CC(C)C)CO2] | 1.4 | 619.319 | 620.4 | A |
| 321 | 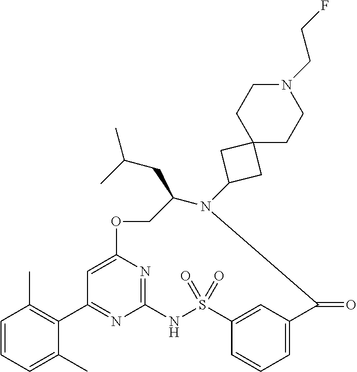 [SMILES: Cc1cccc(C)c1-c1cc2nc(n1)NS(=O)(=O)c1cccc(c1)C(=O)N(C1CC3(CCN(CCF)CC3)C1)[C@H](CC(C)C)CO2] | 1.29 | 649.31 | 650.6 | A |
| 322 | 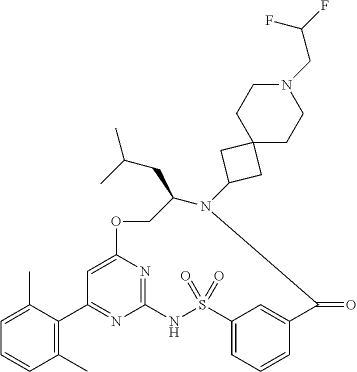 [SMILES: Cc1cccc(C)c1-c1cc2nc(n1)NS(=O)(=O)c1cccc(c1)C(=O)N(C1CC3(CCN(CC(F)F)CC3)C1)[C@H](CC(C)C)CO2] | 1.29 | 667.3 | 668.6 | A |
| 323 | 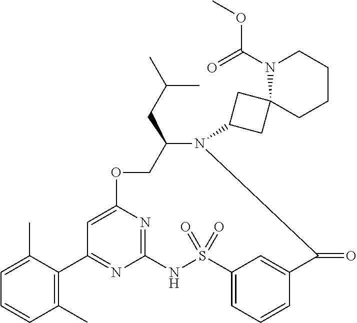 | 1.94 | 661.293 | 662.3 | A |
| 324 | 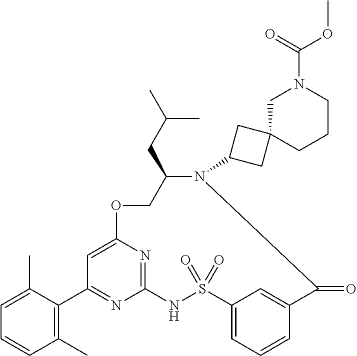 | 1.91 | 661.293 | 662.5 | A |
| 325 | 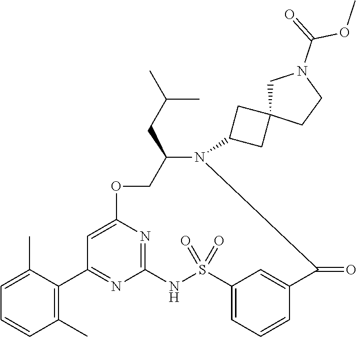 | 1.78 | 647.278 | 648.4 | A |
| 326 | 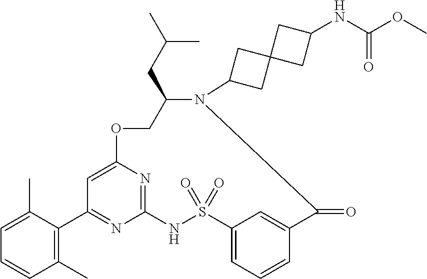 [SMILES: COC(=O)NC1CC2(C1)CC(N1C(=O)c3cccc(c3)S(=O)(=O)Nc3nc(cc(-c4c(C)cccc4C)n3)OC[C@H]1CC(C)C)C2] | 1.73 | 647.278 | 648.3 | A |
| 327 | 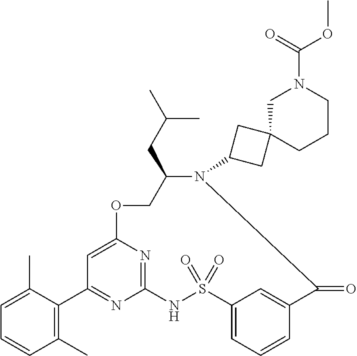 | 1.85 | 661.293 | 662.3 | A |
| 328 | 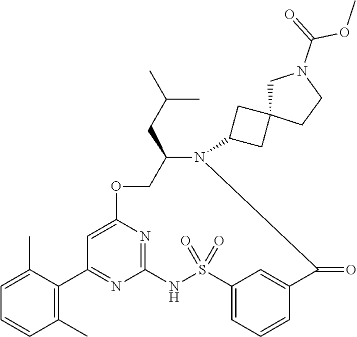 | 1.75 | 647.278 | 648.5 | A |
| 329 | 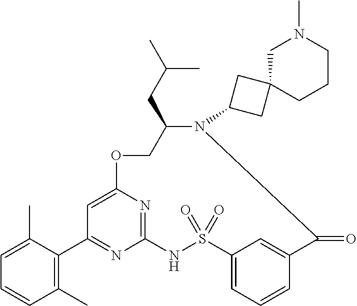 [SMILES: Cc1cccc(C)c1-c1cc2nc(n1)NS(=O)(=O)c1cccc(c1)C(=O)N([C@H]1C[C@]3(CCCN(C)C3)C1)[C@H](CC(C)C)CO2] | 1.38 | 617.304 | 618.4 | A |
| 330 | 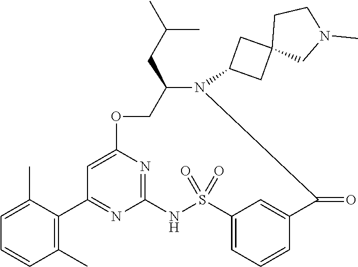 [SMILES: Cc1cccc(C)c1-c1cc2nc(n1)NS(=O)(=O)c1cccc(c1)C(=O)N([C@H]1C[C@]3(CCN(C)C3)C1)[C@H](CC(C)C)CO2] | 1.29 | 603.288 | 604.4 | A |
| 331 | 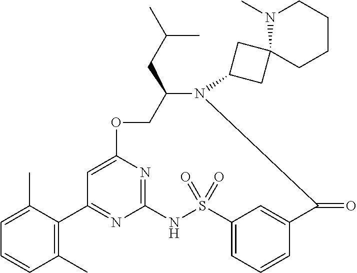 [SMILES: Cc1cccc(C)c1-c1cc2nc(n1)NS(=O)(=O)c1cccc(c1)C(=O)N([C@H]1C[C@]3(CCCCN3C)C1)[C@H](CC(C)C)CO2] | 1.26 | 617.304 | 618.5 | A |
| 332 | 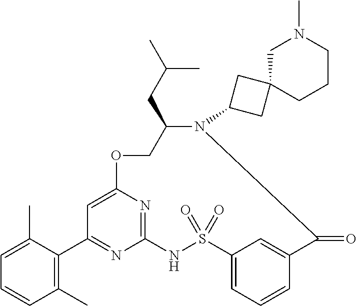 [SMILES: Cc1cccc(C)c1-c1cc2nc(n1)NS(=O)(=O)c1cccc(c1)C(=O)N([C@H]1C[C@]3(CCCN(C)C3)C1)[C@H](CC(C)C)CO2] | 1.29 | 617.304 | 618.5 | A |
| 333 | 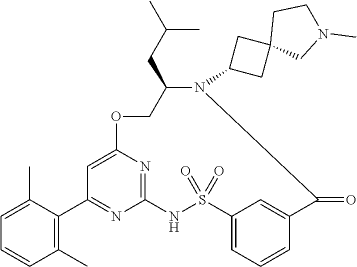 [SMILES: Cc1cccc(C)c1-c1cc2nc(n1)NS(=O)(=O)c1cccc(c1)C(=O)N([C@H]1C[C@]3(CCN(C)C3)C1)[C@H](CC(C)C)CO2] | 1.23 | 603.288 | 604.5 | A |
| 334 | 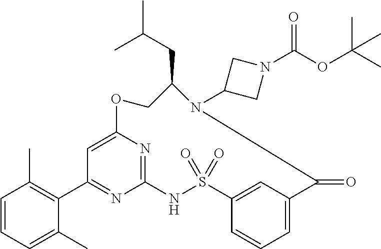 [SMILES: Cc1cccc(C)c1-c1cc2nc(n1)NS(=O)(=O)c1cccc(c1)C(=O)N(C1CN(C(=O)OC(C)(C)C)C1)[C@H](CC(C)C)CO2] | 1.87 | 635.278 | 636.3 | A |
| 3 | 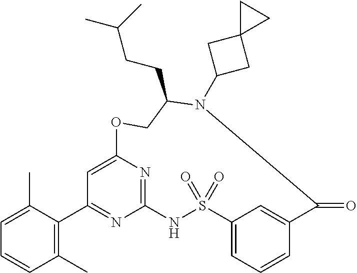 [SMILES: Cc1cccc(C)c1-c1cc2nc(n1)NS(=O)(=O)c1cccc(c1)C(=O)N(C1CC3(CC3)C1)[C@H](CCC(C)C)CO2] | 2.11 | 574.261 | 575.4 | A |
| 335 | 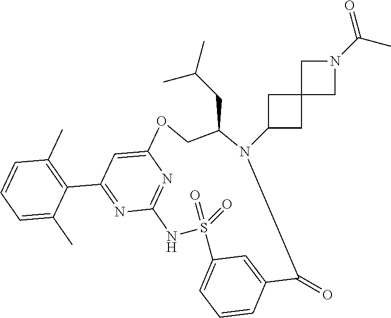 [SMILES: CC(=O)N1CC2(CC(N3C(=O)c4cccc(c4)S(=O)(=O)Nc4nc(cc(-c5c(C)cccc5C)n4)OC[C@H]3CC(C)C)C2)C1] | 1.52 | 617.267 | 618.5 | A |
| 336 | 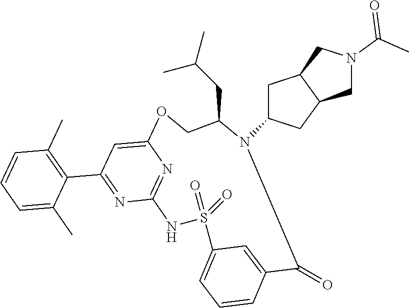 [SMILES: CC(=O)N1C[C@H]2C[C@H](N3C(=O)c4cccc(c4)S(=O)(=O)Nc4nc(cc(-c5c(C)cccc5C)n4)OC[C@H]3CC(C)C)C[C@H]2C1] | 1.6 | 631.283 | 632.5 | A |
| 337 | 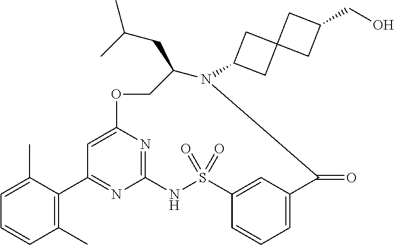 [SMILES: Cc1cccc(C)c1-c1cc2nc(n1)NS(=O)(=O)c1cccc(c1)C(=O)N([C@H]1CC3(C[C@@H](CO)C3)C1)[C@H](CC(C)C)CO2] | 1.7 | 604.272 | 605.4 | A |
| 338 | 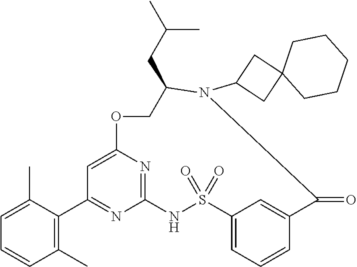 [SMILES: Cc1cccc(C)c1-c1cc2nc(n1)NS(=O)(=O)c1cccc(c1)C(=O)N(C1CC3(CCCCC3)C1)[C@H](CC(C)C)CO2] | 2.32 | 602.293 | 603.5 | A |
| 339 | 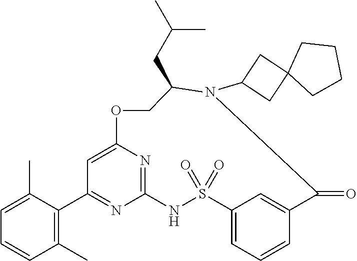 [SMILES: Cc1cccc(C)c1-c1cc2nc(n1)NS(=O)(=O)c1cccc(c1)C(=O)N(C1CC3(CCCC3)C1)[C@H](CC(C)C)CO2] | 2.24 | 588.277 | 589.5 | A |
| 340 | 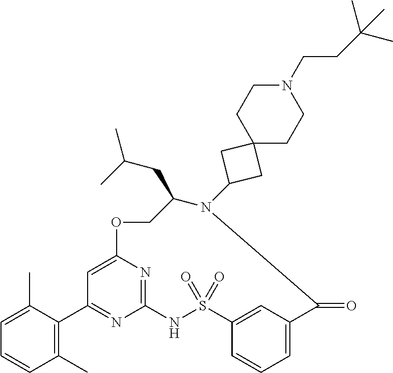 [SMILES: Cc1cccc(C)c1-c1cc2nc(n1)NS(=O)(=O)c1cccc(c1)C(=O)N(C1CC3(CCN(CCC(C)(C)C)CC3)C1)[C@H](CC(C)C)CO2] | 1.5 | 687.382 | 688.7 | A |
| 341 | 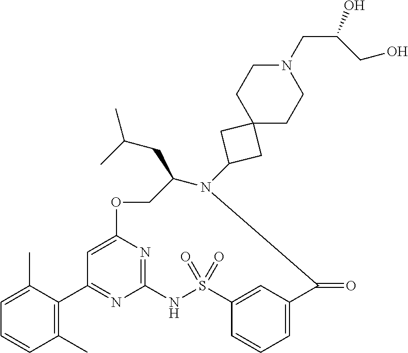 [SMILES: Cc1cccc(C)c1-c1cc2nc(n1)NS(=O)(=O)c1cccc(c1)C(=O)N(C1CC3(CCN(C[C@H](O)CO)CC3)C1)[C@H](CC(C)C)CO2] | 1.21 | 677.325 | 678.6 | A |

TABLE 5
LCMS data of new compounds

| Compound | | LCMS | Calc. | | LCMS |
|---|---|---|---|---|---|
| number | Structure | Rt (min) | mass | M + 1 | Method |
| 342 | 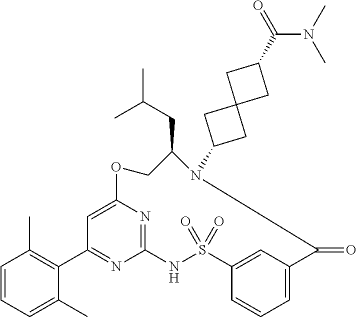 [SMILES: Cc1cccc(C)c1-c1cc2nc(n1)NS(=O)(=O)c1cccc(c1)C(=O)N([C@H]1CC3(C[C@H](C(=O)N(C)C)C3)C1)[C@H](CC(C)C)CO2] | 1.74 | 645.298 | 646.8 | A |
| 343 | 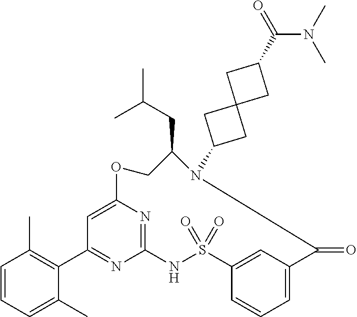 [SMILES: Cc1cccc(C)c1-c1cc2nc(n1)NS(=O)(=O)c1cccc(c1)C(=O)N([C@H]1CC3(C[C@H](C(=O)N(C)C)C3)C1)[C@H](CC(C)C)CO2] | 1.7 | 645.298 | 646.5 | A |
| 344 | 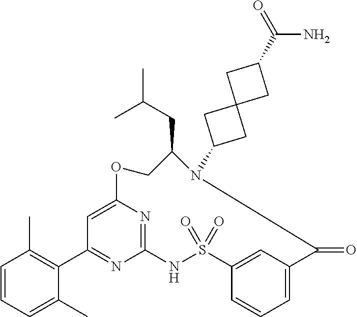 [SMILES: Cc1cccc(C)c1-c1cc2nc(n1)NS(=O)(=O)c1cccc(c1)C(=O)N([C@H]1CC3(C[C@H](C(N)=O)C3)C1)[C@H](CC(C)C)CO2] | 1.53 | 617.267 | 618.5 | A |
| 345 | 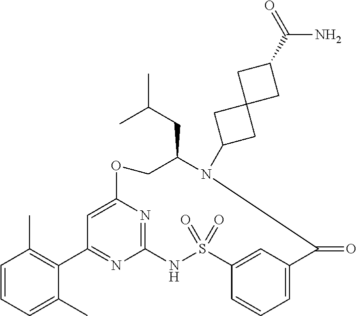 | 2.11 | 617.267 | 618.5 | I |
| 346 | 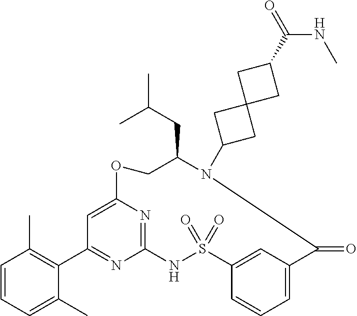 | 1.59 | 631.283 | 632.5 | A |
| 347 | 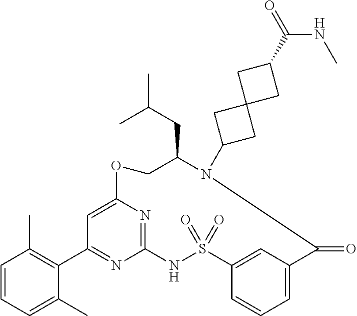 | 2.21 | 631.283 | 632.5 | I |
| 348 | 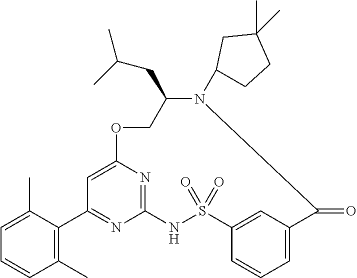 [SMILES: Cc1cccc(C)c1-c1cc2nc(n1)NS(=O)(=O)c1cccc(c1)C(=O)N(C1CCC(C)(C)C1)[C@H](CC(C)C)CO2] | 2.16 | 576.277 | 577.5 | A |
| 349 | 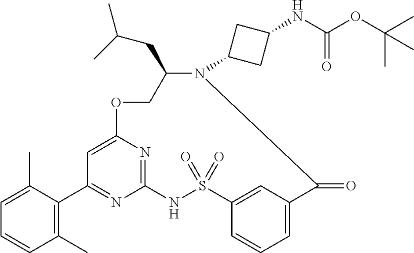 [SMILES: Cc1cccc(C)c1-c1cc2nc(n1)NS(=O)(=O)c1cccc(c1)C(=O)N([C@H]1C[C@@H](NC(=O)OC(C)(C)C)C1)[C@H](CC(C)C)CO2] | 1.93 | 649.293 | 650.5 | A |
| 350 | 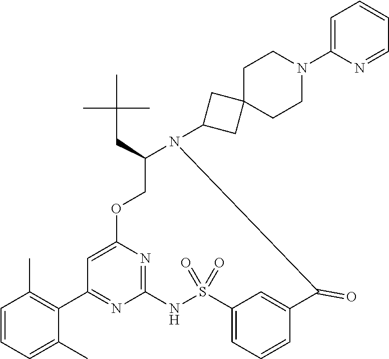 [SMILES: Cc1cccc(C)c1-c1cc2nc(n1)NS(=O)(=O)c1cccc(c1)C(=O)N(C1CC3(CCN(c4ccccn4)CC3)C1)[C@H](CC(C)(C)C)CO2] | 1.42 | 694.33 | 695.8 | A |
| 51 | 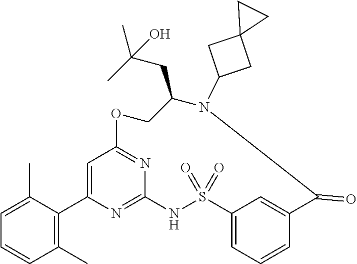 [SMILES: Cc1cccc(C)c1-c1cc2nc(n1)NS(=O)(=O)c1cccc(c1)C(=O)N(C1CC3(CC3)C1)[C@H](CC(C)(C)O)CO2] | 4.02 | 576.241 | 577.3 | S |
| 52 | 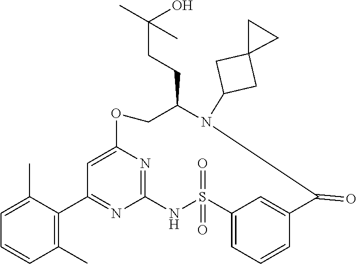 [SMILES: Cc1cccc(C)c1-c1cc2nc(n1)NS(=O)(=O)c1cccc(c1)C(=O)N(C1CC3(CC3)C1)[C@H](CCC(C)(C)O)CO2] | 3.97 | 590.256 | 591.3 | S |
| 136 | 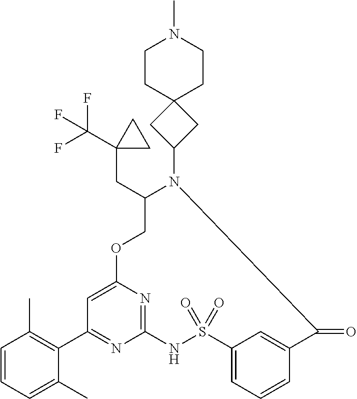 [SMILES: Cc1cccc(C)c1-c1cc2nc(n1)NS(=O)(=O)c1cccc(c1)C(=O)N(C1CC3(CCN(C)CC3)C1)C(CC1(C(F)(F)F)CC1)CO2] | 1.28 | 683.275 | 684.2 | A |
| 137 | 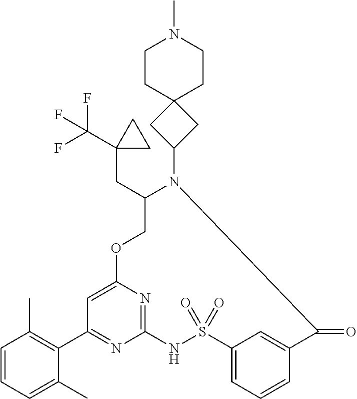 [SMILES: Cc1cccc(C)c1-c1cc2nc(n1)NS(=O)(=O)c1cccc(c1)C(=O)N(C1CC3(CCN(C)CC3)C1)C(CC1(C(F)(F)F)CC1)CO2] | 1.28 | 683.275 | 684.2 | A |
| 351 | 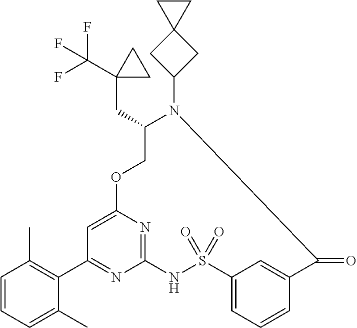 [SMILES: Cc1cccc(C)c1-c1cc2nc(n1)NS(=O)(=O)c1cccc(c1)C(=O)N(C1CC3(CC3)C1)[C@@H](CC1(C(F)(F)F)CC1)CO2] | 1.975 | 626.217 | 627.1 | A |
| 352 | 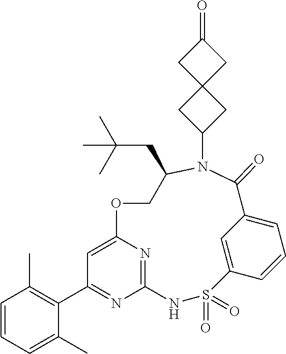 [SMILES: Cc1cccc(C)c1-c1cc2nc(n1)NS(=O)(=O)c1cccc(c1)C(=O)N(C1CC3(CC(=O)C3)C1)[C@H](CC(C)(C)C)CO2] | 1.76 | 602.256 | 603.2 | A |
| 353 | 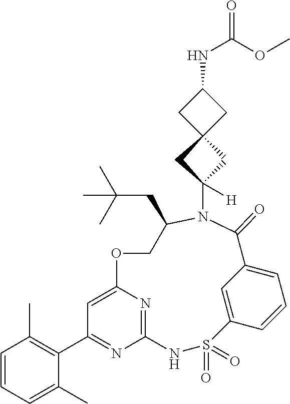 | 1.71 | 659.314 | 660.4 | A |
| 354 | 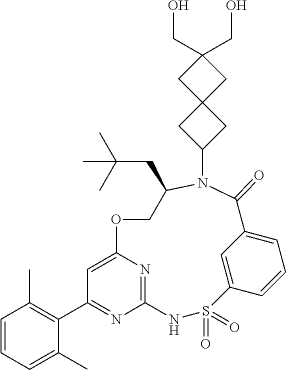 [SMILES: Cc1cccc(C)c1-c1cc2nc(n1)NS(=O)(=O)c1cccc(c1)C(=O)N(C1CC3(C1)CC(CO)(CO)C3)[C@H](CC(C)(C)C)CO2] | 1.66 | 648.298 | 649.3 | A |
| 355 | 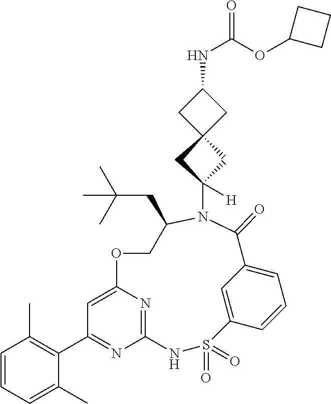 | 2.11 | 701.325 | 702.3 | A |
| 356 | 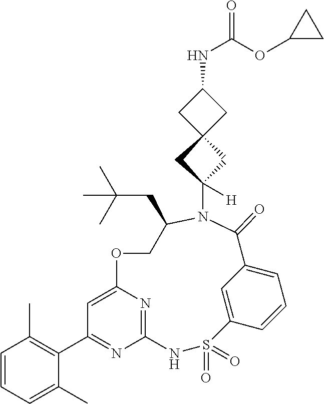 | 1.9 | 687.309 | 688.6 | A |
| 357 | 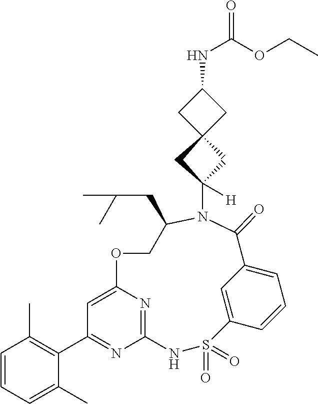 | 1.9 | 675.309 | 676.7 | A |
| 358 | 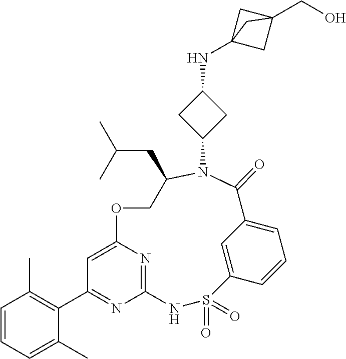 [SMILES: Cc1cccc(C)c1-c1cc2nc(n1)NS(=O)(=O)c1cccc(c1)C(=O)N([C@H]1C[C@@H](NC34CC(CO)(C3)C4)C1)[C@H](CC(C)C)CO2] | 1.23 | 645.298 | 646.3 | A |
| 359 | 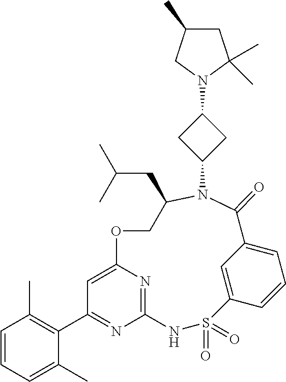 [SMILES: Cc1cccc(C)c1-c1cc2nc(n1)NS(=O)(=O)c1cccc(c1)C(=O)N([C@H]1C[C@@H](N3C[C@@H](C)CC3(C)C)C1)[C@H](CC(C)C)CO2] | 1.39 | 645.335 | 646.2 | A |
| 61 | 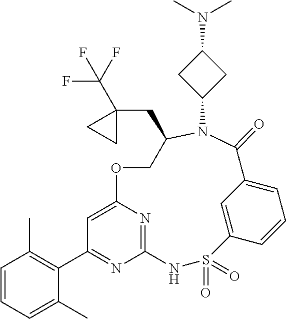 [SMILES: Cc1cccc(C)c1-c1cc2nc(n1)NS(=O)(=O)c1cccc(c1)C(=O)N([C@H]1C[C@@H](N(C)C)C1)[C@H](CC1(C(F)(F)F)CC1)CO2] | 1.18 | 643.244 | 644.2 | A |
| 360 | 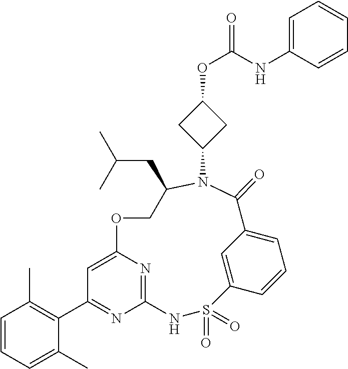 [SMILES: Cc1cccc(C)c1-c1cc2nc(n1)NS(=O)(=O)c1cccc(c1)C(=O)N([C@H]1C[C@@H](OC(=O)Nc3ccccc3)C1)[C@H](CC(C)C)CO2] | 1.95 | 669.262 | 670.1 | A |
| 361 | 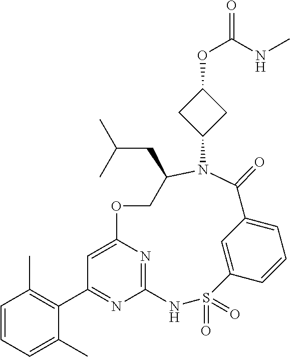 [SMILES: CNC(=O)O[C@H]1C[C@@H](N2C(=O)c3cccc(c3)S(=O)(=O)Nc3nc(cc(-c4c(C)cccc4C)n3)OC[C@H]2CC(C)C)C1] | 1.62 | 607.246 | 608.1 | A |
| 60 | 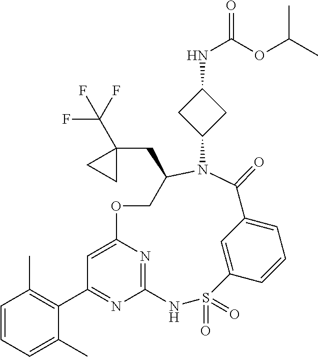 [SMILES: Cc1cccc(C)c1-c1cc2nc(n1)NS(=O)(=O)c1cccc(c1)C(=O)N([C@H]1C[C@@H](NC(=O)OC(C)C)C1)[C@H](CC1(C(F)(F)F)CC1)CO2] | 1.8 | 701.249 | 702.1 | A |
| 59 | 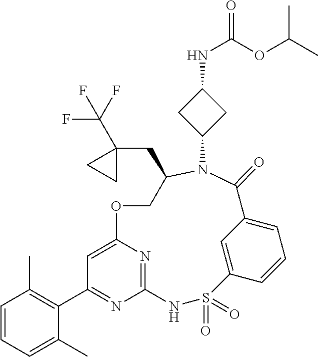 [SMILES: Cc1cccc(C)c1-c1cc2nc(n1)NS(=O)(=O)c1cccc(c1)C(=O)N([C@H]1C[C@@H](NC(=O)OC(C)C)C1)[C@H](CC1(C(F)(F)F)CC1)CO2] | 1.76 | 701.249 | 702.1 | A |
| 362 | 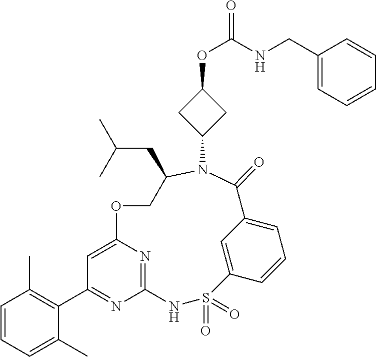 [SMILES: Cc1cccc(C)c1-c1cc2nc(n1)NS(=O)(=O)c1cccc(c1)C(=O)N([C@H]1C[C@H](OC(=O)NCc3ccccc3)C1)[C@H](CC(C)C)CO2] | 1.9 | 683.278 | 684.1 | A |
| 363 | 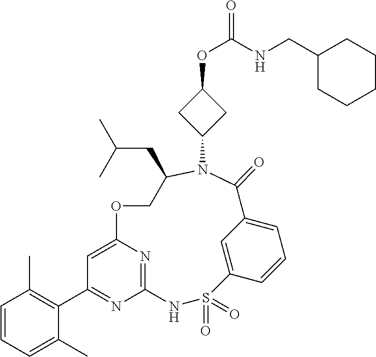 [SMILES: Cc1cccc(C)c1-c1cc2nc(n1)NS(=O)(=O)c1cccc(c1)C(=O)N([C@H]1C[C@H](OC(=O)NCC3CCCCC3)C1)[C@H](CC(C)C)CO2] | 2.09 | 689.325 | 690.2 | A |
| 364 | 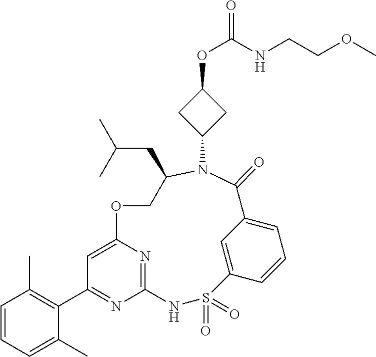 [SMILES: COCCNC(=O)O[C@H]1C[C@H](N2C(=O)c3cccc(c3)S(=O)(=O)Nc3nc(cc(-c4c(C)cccc4C)n3)OC[C@H]2CC(C)C)C1] | 1.66 | 651.273 | 652.2 | A |
| 365 | 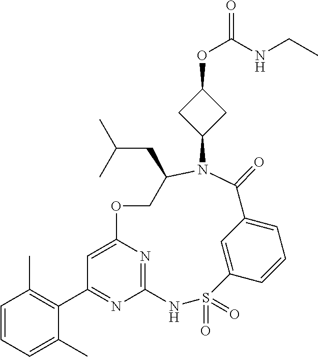 [SMILES: CCNC(=O)O[C@H]1C[C@@H](N2C(=O)c3cccc(c3)S(=O)(=O)Nc3nc(cc(-c4c(C)cccc4C)n3)OC[C@H]2CC(C)C)C1] | 1.72 | 621.262 | 622.1 | A |
| 366 | 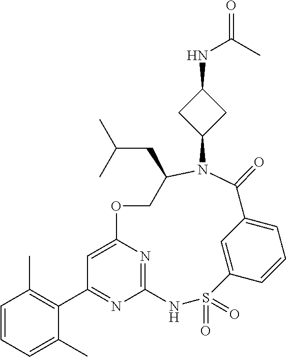 [SMILES: CC(=O)N[C@H]1C[C@@H](N2C(=O)c3cccc(c3)S(=O)(=O)Nc3nc(cc(-c4c(C)cccc4C)n3)OC[C@H]2CC(C)C)C1] | 1.47 | 591.252 | 592.2 | A |
| 367 | 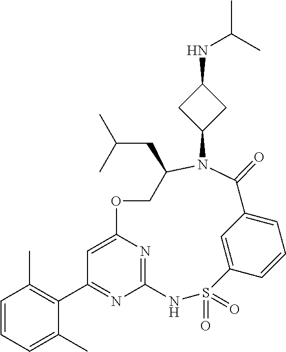 [SMILES: Cc1cccc(C)c1-c1cc2nc(n1)NS(=O)(=O)c1cccc(c1)C(=O)N([C@H]1C[C@@H](NC(C)C)C1)[C@H](CC(C)C)CO2] | 1.22 | 591.288 | 592.2 | A |
| 368 | 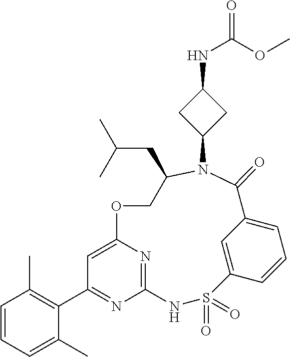 [SMILES: COC(=O)N[C@H]1C[C@@H](N2C(=O)c3cccc(c3)S(=O)(=O)Nc3nc(cc(-c4c(C)cccc4C)n3)OC[C@H]2CC(C)C)C1] | 1.62 | 607.246 | 608.2 | A |
| 369 | 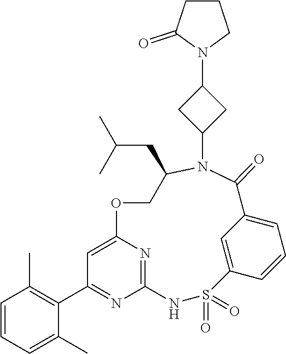 [SMILES: Cc1cccc(C)c1-c1cc2nc(n1)NS(=O)(=O)c1cccc(c1)C(=O)N(C1CC(N3CCCC3=O)C1)[C@H](CC(C)C)CO2] | 1.58 | 617.267 | 618.1 | A |
| 370 | 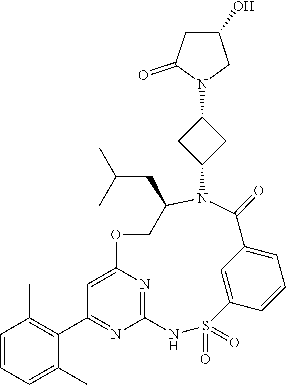 [SMILES: Cc1cccc(C)c1-c1cc2nc(n1)NS(=O)(=O)c1cccc(c1)C(=O)N([C@H]1C[C@@H](N3C[C@@H](O)CC3=O)C1)[C@H](CC(C)C)CO2] | 1.41 | 633.262 | 634.2 | A |
| 371 | 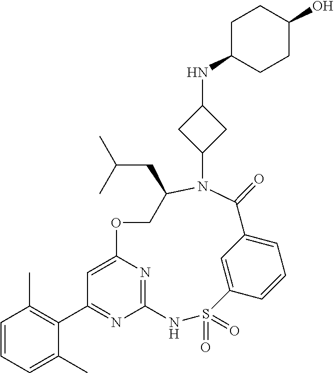 [SMILES: Cc1cccc(C)c1-c1cc2nc(n1)NS(=O)(=O)c1cccc(c1)C(=O)N(C1CC(N[C@H]3CC[C@@H](O)CC3)C1)[C@H](CC(C)C)CO2] | 1.27 | 647.314 | 648.2 | A |
| 372 | 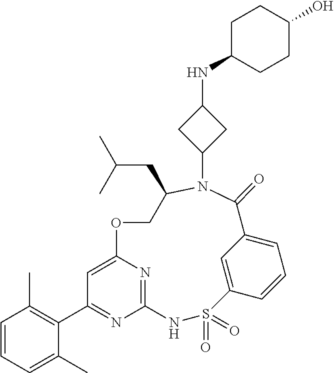 [SMILES: Cc1cccc(C)c1-c1cc2nc(n1)NS(=O)(=O)c1cccc(c1)C(=O)N(C1CC(N[C@H]3CC[C@H](O)CC3)C1)[C@H](CC(C)C)CO2] | 1.22 | 647.314 | 648.2 | A |
| 373 | 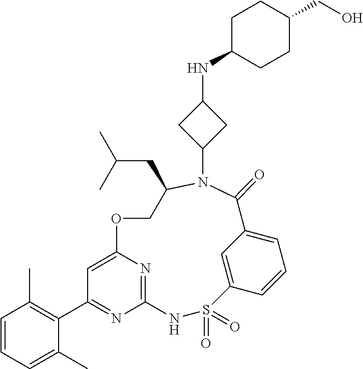 [SMILES: Cc1cccc(C)c1-c1cc2nc(n1)NS(=O)(=O)c1cccc(c1)C(=O)N(C1CC(N[C@H]3CC[C@H](CO)CC3)C1)[C@H](CC(C)C)CO2] | 1.27 | 661.33 | 662.2 | A |
| 374 | 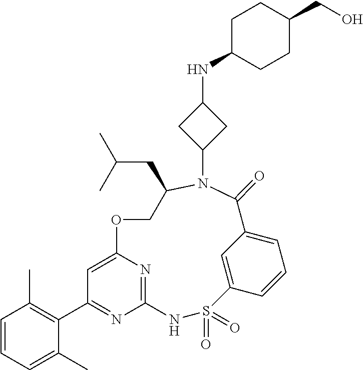 [SMILES: Cc1cccc(C)c1-c1cc2nc(n1)NS(=O)(=O)c1cccc(c1)C(=O)N(C1CC(N[C@H]3CC[C@@H](CO)CC3)C1)[C@H](CC(C)C)CO2] | 1.23 | 633.298 | 634.2 | A |
| 375 | 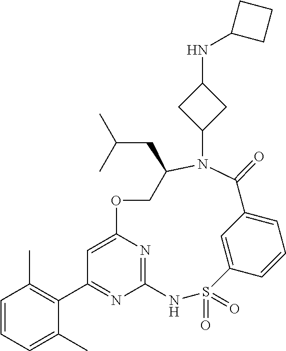 [SMILES: Cc1cccc(C)c1-c1cc2nc(n1)NS(=O)(=O)c1cccc(c1)C(=O)N(C1CC(NC3CCC3)C1)[C@H](CC(C)C)CO2] | 1.32 | 603.288 | 604.2 | A |
| 376 | 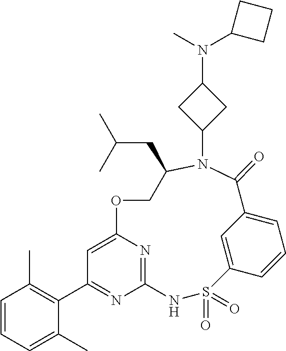 [SMILES: Cc1cccc(C)c1-c1cc2nc(n1)NS(=O)(=O)c1cccc(c1)C(=O)N(C1CC(N(C)C3CCC3)C1)[C@H](CC(C)C)CO2] | 1.31 | 617.304 | 618.2 | A |
| 377 | 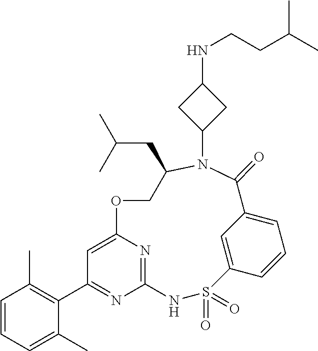 [SMILES: Cc1cccc(C)c1-c1cc2nc(n1)NS(=O)(=O)c1cccc(c1)C(=O)N(C1CC(NCCC(C)C)C1)[C@H](CC(C)C)CO2] | 1.44 | 619.319 | 620.2 | A |
| 378 | 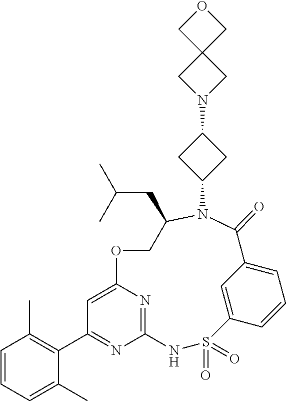 [SMILES: Cc1cccc(C)c1-c1cc2nc(n1)NS(=O)(=O)c1cccc(c1)C(=O)N([C@H]1C[C@@H](N3CC4(COC4)C3)C1)[C@H](CC(C)C)CO2] | 1.21 | 631.283 | 632.2 | A |
| 379 | 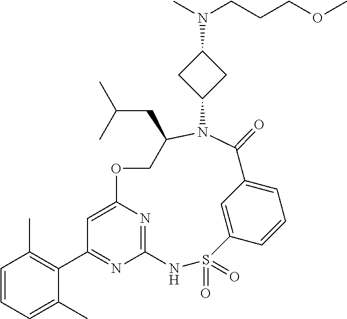 [SMILES: COCCCN(C)[C@H]1C[C@@H](N2C(=O)c3cccc(c3)S(=O)(=O)Nc3nc(cc(-c4c(C)cccc4C)n3)OC[C@H]2CC(C)C)C1] | 1.32 | 635.314 | 636.2 | A |
| 380 | 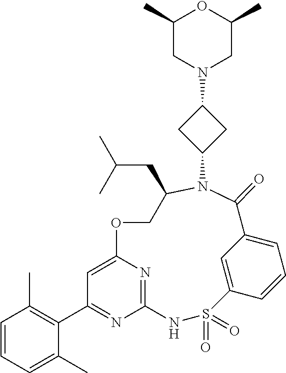 [SMILES: Cc1cccc(C)c1-c1cc2nc(n1)NS(=O)(=O)c1cccc(c1)C(=O)N([C@H]1C[C@@H](N3C[C@@H](C)O[C@@H](C)C3)C1)[C@H](CC(C)C)CO2] | 1.33 | 647.314 | 648.2 | A |
| 381 | 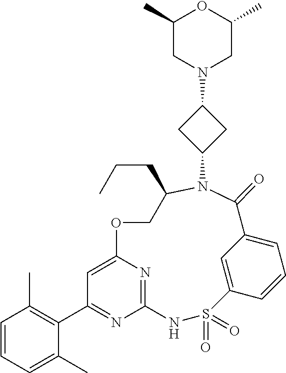 [SMILES: CCC[C@@H]1COc2cc(-c3c(C)cccc3C)nc(n2)NS(=O)(=O)c2cccc(c2)C(=O)N1[C@H]1C[C@@H](N2C[C@@H](C)O[C@H](C)C2)C1] | 1.31 | 647.314 | 648.2 | A |
| 382 | 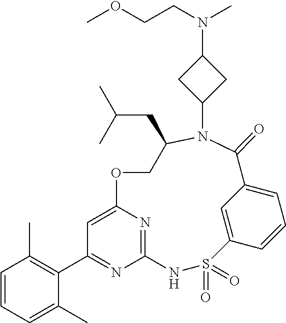 [SMILES: COCCN(C)C1CC(N2C(=O)c3cccc(c3)S(=O)(=O)Nc3nc(cc(-c4c(C)cccc4C)n3)OC[C@H]2CC(C)C)C1] | 1.29 | 621.298 | 622.2 | A |
| 383 | 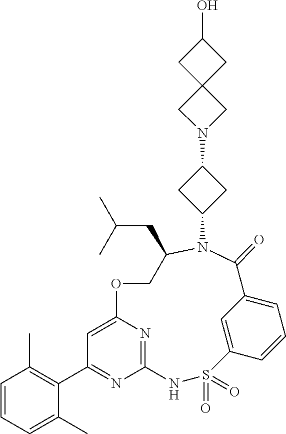 [SMILES: Cc1cccc(C)c1-c1cc2nc(n1)NS(=O)(=O)c1cccc(c1)C(=O)N([C@H]1C[C@@H](N3CC4(CC(O)C4)C3)C1)[C@H](CC(C)C)CO2] | 1.23 | 645.298 | 646.2 | A |
| 66 | 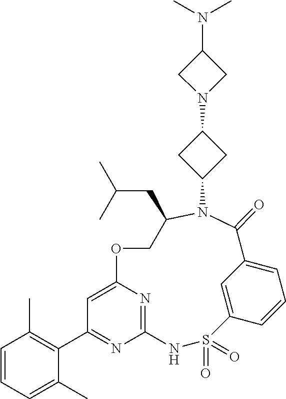 [SMILES: Cc1cccc(C)c1-c1cc2nc(n1)NS(=O)(=O)c1cccc(c1)C(=O)N([C@H]1C[C@@H](N3CC(N(C)C)C3)C1)[C@H](CC(C)C)CO2] | 1.06 | 632.314 | 633.2 | A |
| 384 | 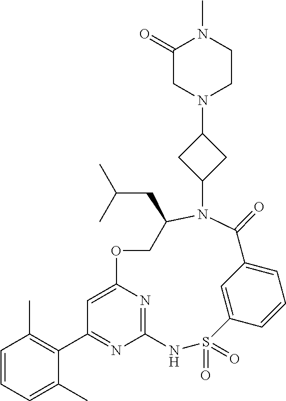 [SMILES: Cc1cccc(C)c1-c1cc2nc(n1)NS(=O)(=O)c1cccc(c1)C(=O)N(C1CC(N3CCN(C)C(=O)C3)C1)[C@H](CC(C)C)CO2] | 1.2 | 646.294 | 647.2 | A |
| 385 | 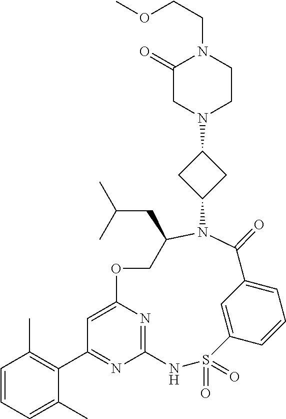 [SMILES: COCCN1CCN([C@H]2C[C@@H](N3C(=O)c4cccc(c4)S(=O)(=O)Nc4nc(cc(-c5c(C)cccc5C)n4)OC[C@H]3CC(C)C)C2)CC1=O] | 1.24 | 690.32 | 691.2 | A |
| 386 | 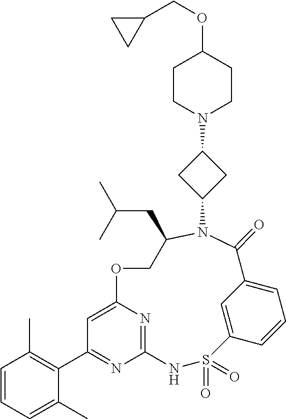 [SMILES: Cc1cccc(C)c1-c1cc2nc(n1)NS(=O)(=O)c1cccc(c1)C(=O)N([C@H]1C[C@@H](N3CCC(OCC4CC4)CC3)C1)[C@H](CC(C)C)CO2] | 1.45 | 687.345 | 688.3 | A |
| 387 | 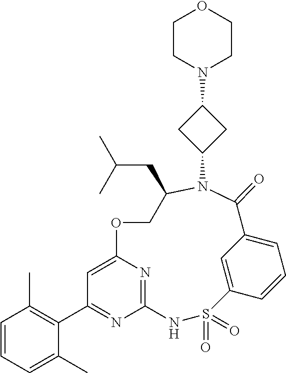 [SMILES: Cc1cccc(C)c1-c1cc2nc(n1)NS(=O)(=O)c1cccc(c1)C(=O)N([C@H]1C[C@@H](N3CCOCC3)C1)[C@H](CC(C)C)CO2] | 1.24 | 619.283 | 620.2 | A |
| 388 | 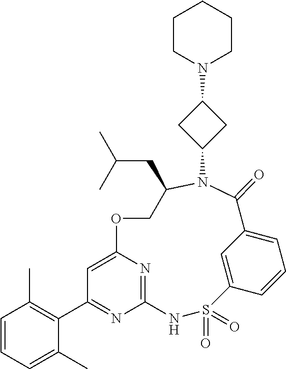 [SMILES: Cc1cccc(C)c1-c1cc2nc(n1)NS(=O)(=O)c1cccc(c1)C(=O)N([C@H]1C[C@@H](N3CCCCC3)C1)[C@H](CC(C)C)CO2] | 1.17 | 617.304 | 618.2 | A |
| 37 | 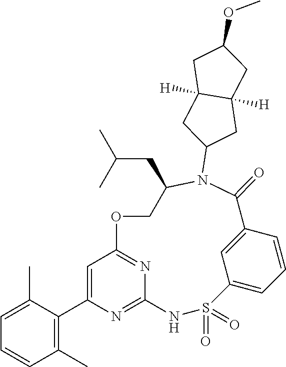 [SMILES: CO[C@H]1C[C@H]2CC(N3C(=O)c4cccc(c4)S(=O)(=O)Nc4nc(cc(-c5c(C)cccc5C)n4)OC[C@H]3CC(C)C)C[C@H]2C1] | 2.03 | 618.288 | 619.1 | A |
| 389 | 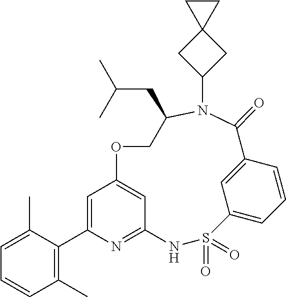 [SMILES: Cc1cccc(C)c1-c1cc2cc(n1)NS(=O)(=O)c1cccc(c1)C(=O)N(C1CC3(CC3)C1)[C@H](CC(C)C)CO2] | 2.01 | 559.25 | 560.3 | A |
| 390 | 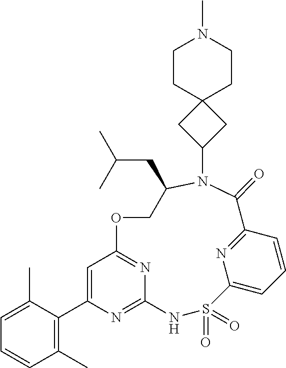 [SMILES: Cc1cccc(C)c1-c1cc2nc(n1)NS(=O)(=O)c1cccc(n1)C(=O)N(C1CC3(CCN(C)CC3)C1)[C@H](CC(C)C)CO2] | 1.25 | 618.299 | 619.6 | A |
| 391 | 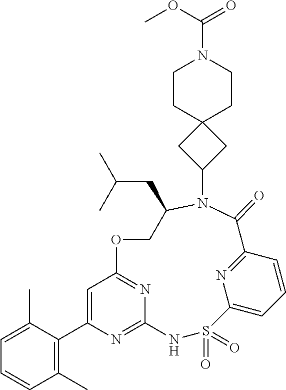 [SMILES: COC(=O)N1CCC2(CC1)CC(N1C(=O)c3cccc(n3)S(=O)(=O)Nc3nc(cc(-c4c(C)cccc4C)n3)OC[C@H]1CC(C)C)C2] | 1.79 | 662.289 | 663.4 | A |
| 392 | 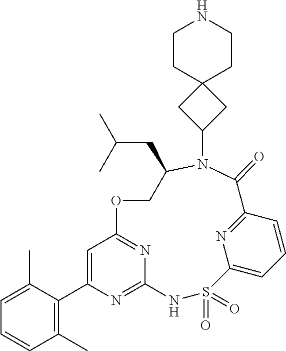 [SMILES: Cc1cccc(C)c1-c1cc2nc(n1)NS(=O)(=O)c1cccc(n1)C(=O)N(C1CC3(CCNCC3)C1)[C@H](CC(C)C)CO2] | 1.2 | 604.283 | 605.6 | A |
| 393 | 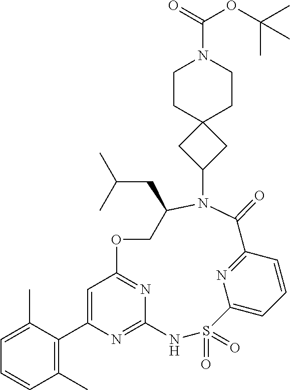 [SMILES: Cc1cccc(C)c1-c1cc2nc(n1)NS(=O)(=O)c1cccc(n1)C(=O)N(C1CC3(CCN(C(=O)OC(C)(C)C)CC3)C1)[C@H](CC(C)C)CO2] | 2.08 | 704.336 | 705.5 | A |
| 394 | 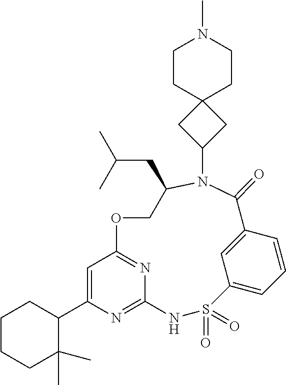 [SMILES: CC(C)C[C@@H]1COc2cc(C3CCCCC3(C)C)nc(n2)NS(=O)(=O)c2cccc(c2)C(=O)N1C1CC2(CCN(C)CC2)C1] | 1.4 | 623.351 | 624.5 | A |
| 395 | 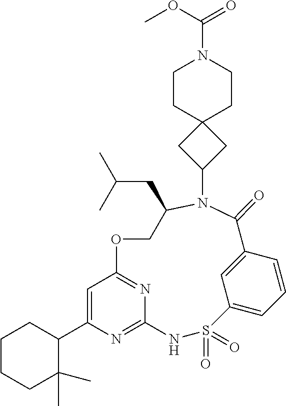 [SMILES: COC(=O)N1CCC2(CC1)CC(N1C(=O)c3cccc(c3)S(=O)(=O)Nc3nc(cc(C4CCCCC4(C)C)n3)OC[C@H]1CC(C)C)C2] | 2.03 | 667.34 | 668.5 | A |
| 396 | 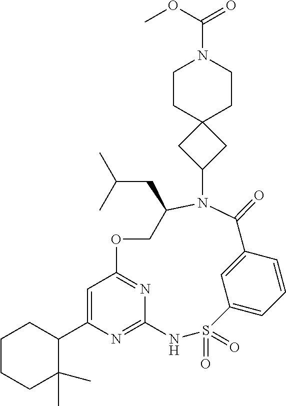 [SMILES: COC(=O)N1CCC2(CC1)CC(N1C(=O)c3cccc(c3)S(=O)(=O)Nc3nc(cc(C4CCCCC4(C)C)n3)OC[C@H]1CC(C)C)C2] | 2 | 667.34 | 668.5 | A |
| 397 | 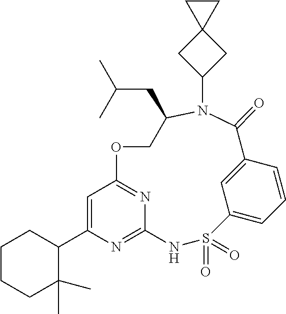 [SMILES: CC(C)C[C@@H]1COc2cc(C3CCCCC3(C)C)nc(n2)NS(=O)(=O)c2cccc(c2)C(=O)N1C1CC2(CC2)C1] | 2.21 | 580.308 | 581.4 | A |
| 398 | 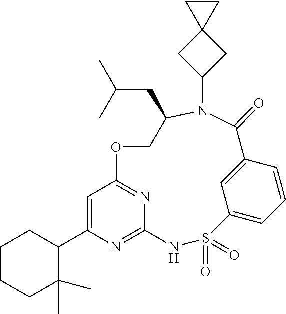 [SMILES: CC(C)C[C@@H]1COc2cc(C3CCCCC3(C)C)nc(n2)NS(=O)(=O)c2cccc(c2)C(=O)N1C1CC2(CC2)C1] | 2.18 | 580.308 | 581.5 | A |
| 399 | 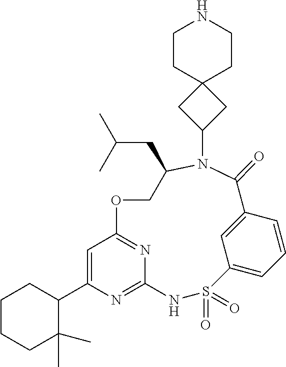 [SMILES: CC(C)C[C@@H]1COc2cc(C3CCCCC3(C)C)nc(n2)NS(=O)(=O)c2cccc(c2)C(=O)N1C1CC2(CCNCC2)C1] | 1.43 | 609.335 | 610.5 | A |
| 400 | 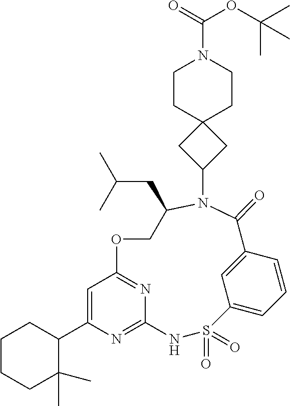 [SMILES: CC(C)C[C@@H]1COc2cc(C3CCCCC3(C)C)nc(n2)NS(=O)(=O)c2cccc(c2)C(=O)N1C1CC2(CCN(C(=O)OC(C)(C)C)CC2)C1] | 2.33 | 709.387 | 710.5 | A |
| 401 | 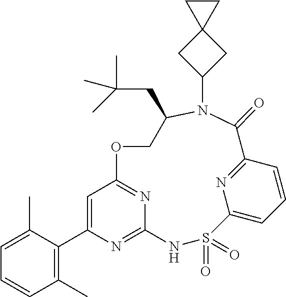 [SMILES: Cc1cccc(C)c1-c1cc2nc(n1)NS(=O)(=O)c1cccc(n1)C(=O)N(C1CC3(CC3)C1)[C@H](CC(C)(C)C)CO2] | 1.95 | 575.257 | 576.3 | A |
| 402 | 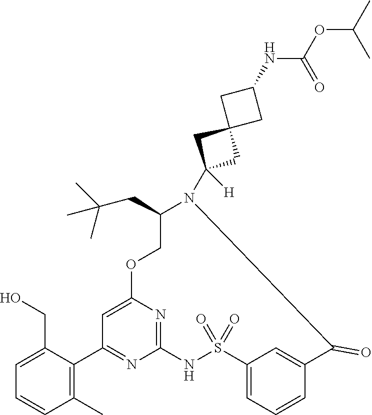 | 1.8 | 705.32 | 706.41 | A |
| 403 | 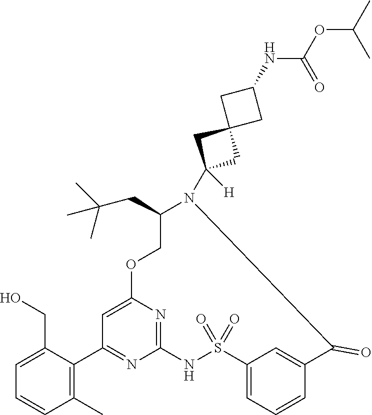 | 1.79 | 705.32 | 706.41 | A |
| 4 | 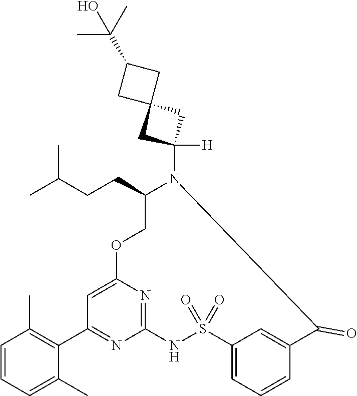 | 1.96 | 646.319 | 647.35 | A |
| 5 | 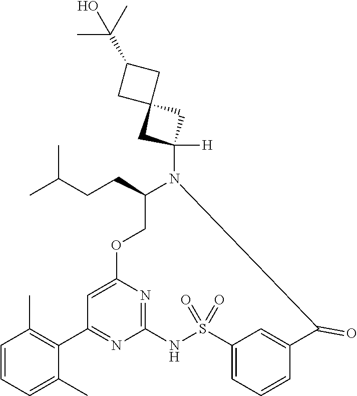 | 2 | 646.319 | 647.35 | A |
| 404 | 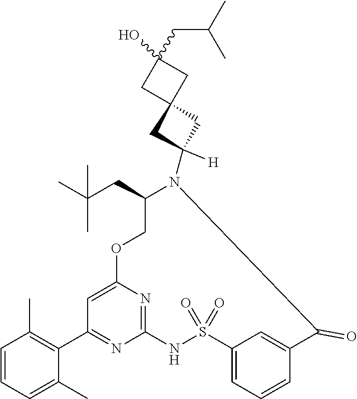 | 2.02 | 660.335 | 661.4 | A |
| 405 | 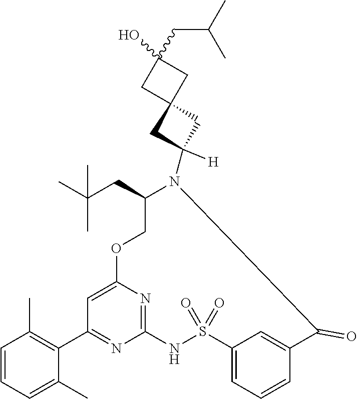 | 2.11 | 660.335 | 661.34 | A |
| 408 | 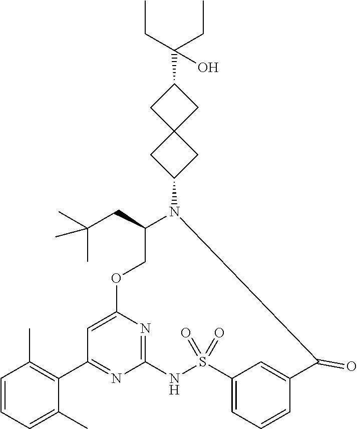 [SMILES: CCC(O)(CC)[C@H]1CC2(C[C@H](N3C(=O)c4cccc(c4)S(=O)(=O)Nc4nc(cc(-c5c(C)cccc5C)n4)OC[C@H]3CC(C)(C)C)C2)C1] | 2.13 | 674.35 | 675.33 | A |
| 409 | 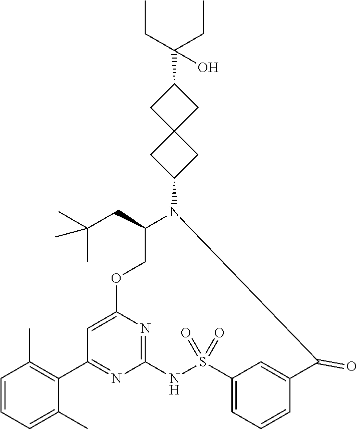 [SMILES: CCC(O)(CC)[C@H]1CC2(C[C@H](N3C(=O)c4cccc(c4)S(=O)(=O)Nc4nc(cc(-c5c(C)cccc5C)n4)OC[C@H]3CC(C)(C)C)C2)C1] | 2.22 | 674.35 | 675.39 | A |
| 410 | 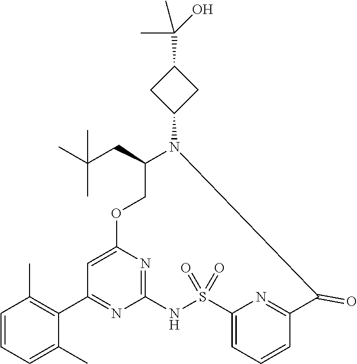 [SMILES: Cc1cccc(C)c1-c1cc2nc(n1)NS(=O)(=O)c1cccc(n1)C(=O)N([C@H]1C[C@@H](C(C)(C)O)C1)[C@H](CC(C)(C)C)CO2] | 1.66 | 607.283 | 608.25 | A |
| 411 | 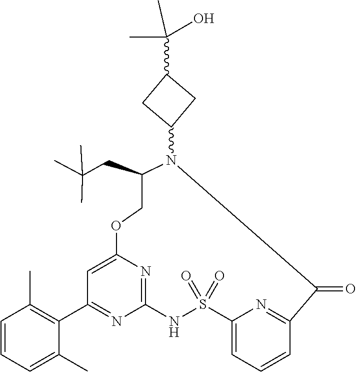 | 1.78 | 607.283 | 608.25 | A |
| 412 | 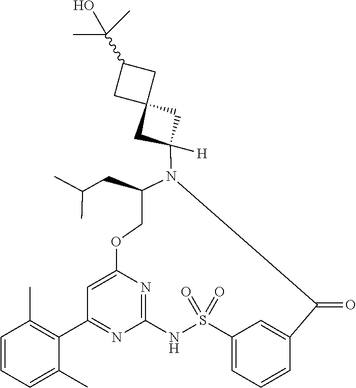 | 1.89 | 632.303 | 633.3 | A |
| 413 | 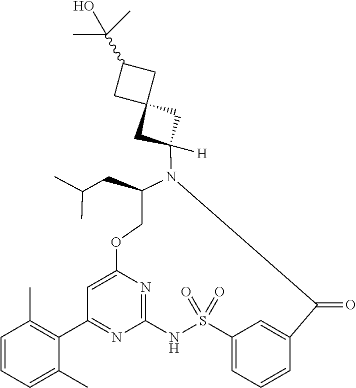 | 1.93 | 632.303 | 633.3 | A |
| 414 | 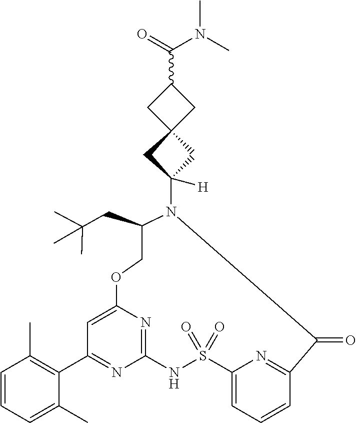 | 1.72 | 660.309 | 661.35 | A |
| 415 | 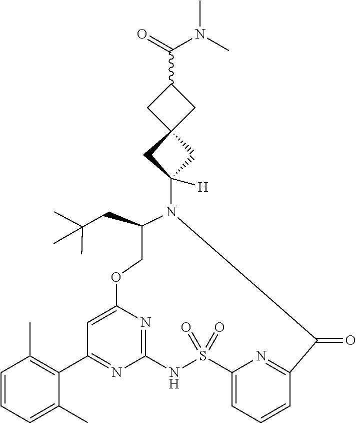 | 1.66 | 660.309 | 661.35 | A |
| 416 | 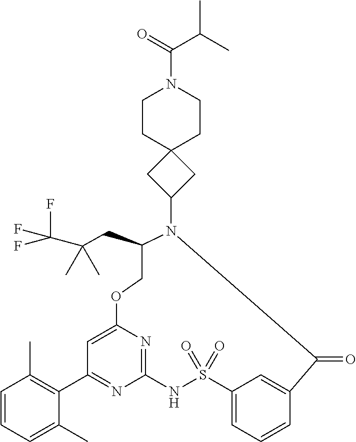 [SMILES: Cc1cccc(C)c1-c1cc2nc(n1)NS(=O)(=O)c1cccc(c1)C(=O)N(C1CC3(CCN(C(=O)C(C)C)CC3)C1)[C@H](CC(C)(C)C(F)(F)F)CO2] | 1.89 | 741.317 | 742.8 | A |
| 417 | 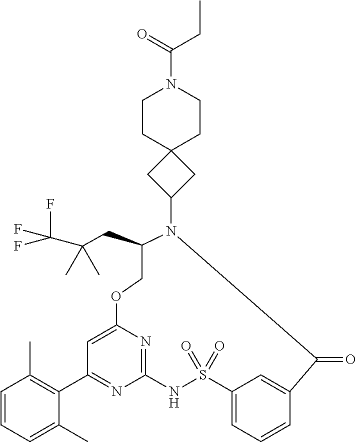 [SMILES: CCC(=O)N1CCC2(CC1)CC(N1C(=O)c3cccc(c3)S(=O)(=O)Nc3nc(cc(-c4c(C)cccc4C)n3)OC[C@H]1CC(C)(C)C(F)(F)F)C2] | 1.8 | 727.302 | 728.65 | A |
| 418 | 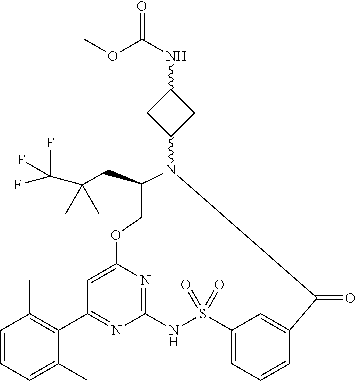 [SMILES: COC(=O)NC1CC(N2C(=O)c3cccc(c3)S(=O)(=O)Nc3nc(cc(-c4c(C)cccc4C)n3)OC[C@H]2CC(C)(C)C(F)(F)F)C1] | 1.65 | 675.234 | 676.66 | A |
| 419 | 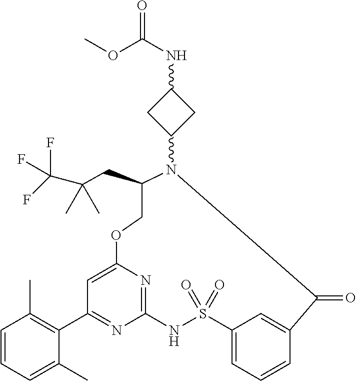 [SMILES: COC(=O)NC1CC(N2C(=O)c3cccc(c3)S(=O)(=O)Nc3nc(cc(-c4c(C)cccc4C)n3)OC[C@H]2CC(C)(C)C(F)(F)F)C1] | 1.69 | 675.234 | 676.62 | A |
| 420 | 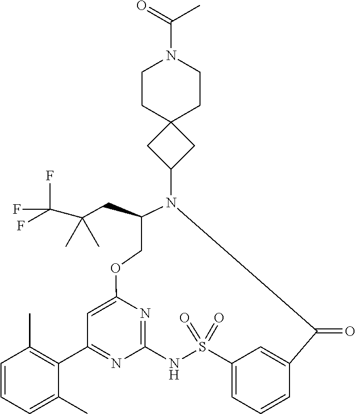 [SMILES: CC(=O)N1CCC2(CC1)CC(N1C(=O)c3cccc(c3)S(=O)(=O)Nc3nc(cc(-c4c(C)cccc4C)n3)OC[C@H]1CC(C)(C)C(F)(F)F)C2] | 1.71 | 713.286 | 714.57 | A |
| 190 | 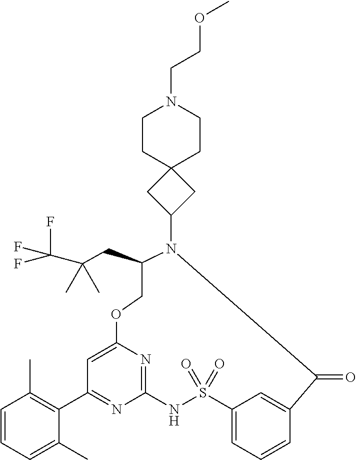 [SMILES: COCCN1CCC2(CC1)CC(N1C(=O)c3cccc(c3)S(=O)(=O)Nc3nc(cc(-c4c(C)cccc4C)n3)OC[C@H]1CC(C)(C)C(F)(F)F)C2] | 1.36 | 729.317 | 730.72 | A |
| 421 | 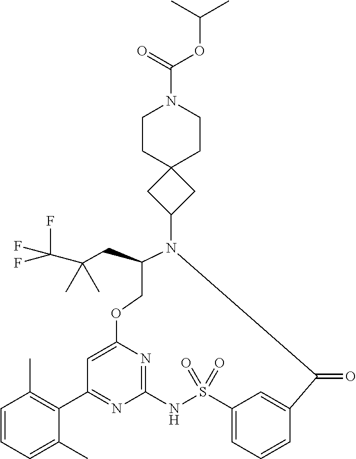 [SMILES: Cc1cccc(C)c1-c1cc2nc(n1)NS(=O)(=O)c1cccc(c1)C(=O)N(C1CC3(CCN(C(=O)OC(C)C)CC3)C1)[C@H](CC(C)(C)C(F)(F)F)CO2] | 2.09 | 757.312 | 758.82 | A |
| 189 | 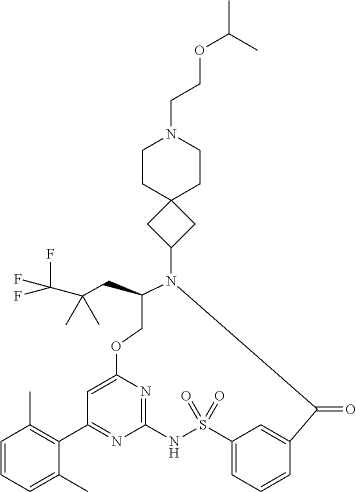 [SMILES: Cc1cccc(C)c1-c1cc2nc(n1)NS(=O)(=O)c1cccc(c1)C(=O)N(C1CC3(CCN(CCOC(C)C)CC3)C1)[C@H](CC(C)(C)C(F)(F)F)CO2] | 1.49 | 757.348 | 758.97 | A |
| 422 | 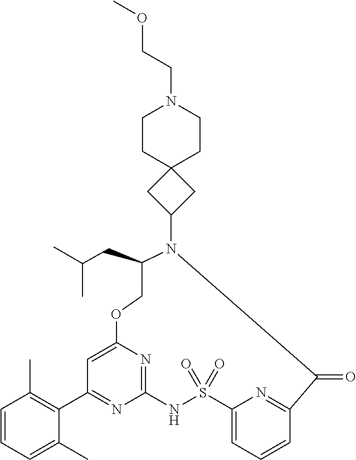 [SMILES: COCCN1CCC2(CC1)CC(N1C(=O)c3cccc(n3)S(=O)(=O)Nc3nc(cc(-c4c(C)cccc4C)n3)OC[C@H]1CC(C)C)C2] | 1.3 | 661.33 | 662.87 | A |
| 423 | 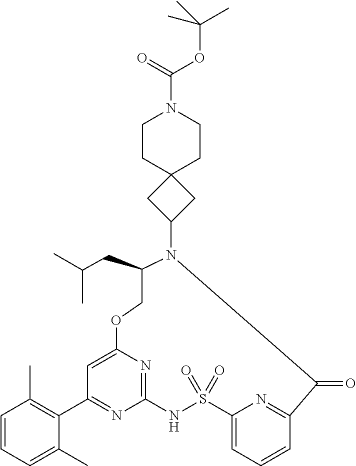 [SMILES: Cc1cccc(C)c1-c1cc2nc(n1)NS(=O)(=O)c1cccc(n1)C(=O)N(C1CC3(CCN(C(=O)OC(C)(C)C)CC3)C1)[C@H](CC(C)C)CO2] | 2.16 | 703.34 | 704.34 | A |
| 424 | 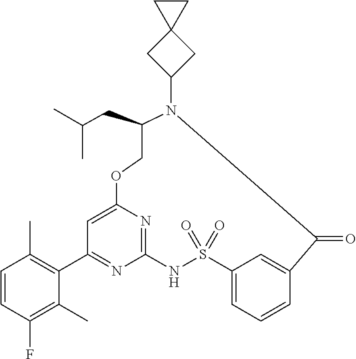 [SMILES: Cc1ccc(F)c(C)c1-c1cc2nc(n1)NS(=O)(=O)c1cccc(c1)C(=O)N(C1CC3(CC3)C1)[C@H](CC(C)C)CO2] | 2.15 | 578.236 | 579.2 | A |
| 425 | 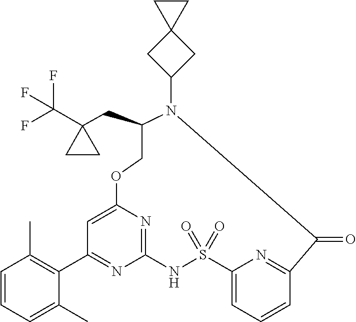 [SMILES: Cc1cccc(C)c1-c1cc2nc(n1)NS(=O)(=O)c1cccc(n1)C(=O)N(C1CC3(CC3)C1)[C@H](CC1(C(F)(F)F)CC1)CO2] | 1.89 | 626.217 | 627.53 | A |
| 426 | 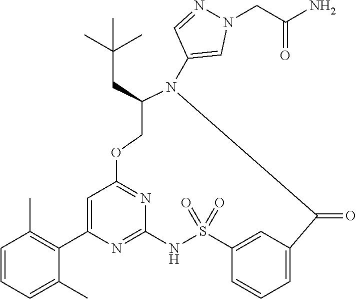 [SMILES: Cc1cccc(C)c1-c1cc2nc(n1)NS(=O)(=O)c1cccc(c1)C(=O)N(c1cnn(CC(N)=O)c1)[C@H](CC(C)(C)C)CO2] | 1.37 | 617.242 | 618.3 | A |
| 427 | 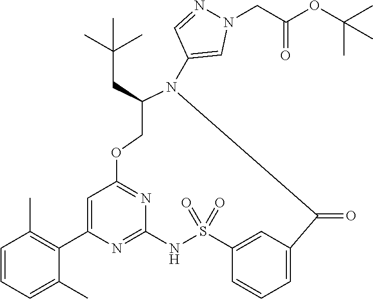 [SMILES: Cc1cccc(C)c1-c1cc2nc(n1)NS(=O)(=O)c1cccc(c1)C(=O)N(c1cnn(CC(=O)OC(C)(C)C)c1)[C@H](CC(C)(C)C)CO2] | 1.86 | 674.289 | 675.3 | A |
| 428 | 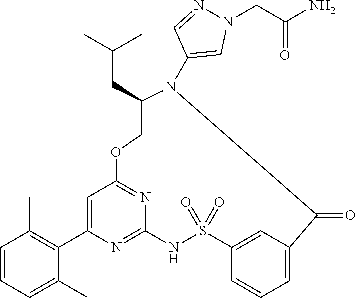 [SMILES: Cc1cccc(C)c1-c1cc2nc(n1)NS(=O)(=O)c1cccc(c1)C(=O)N(c1cnn(CC(N)=O)c1)[C@H](CC(C)C)CO2] | 1.5 | 603.226 | 604.3 | A |
| 429 | 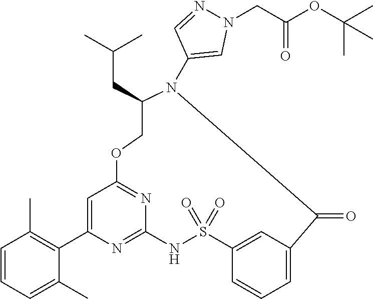 [SMILES: Cc1cccc(C)c1-c1cc2nc(n1)NS(=O)(=O)c1cccc(c1)C(=O)N(c1cnn(CC(=O)OC(C)(C)C)c1)[C@H](CC(C)C)CO2] | 1.83 | 660.273 | 661.3 | A |
| 430 | 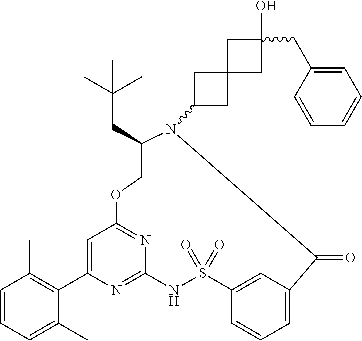 [SMILES: Cc1cccc(C)c1-c1cc2nc(n1)NS(=O)(=O)c1cccc(c1)C(=O)N(C1CC3(C1)CC(O)(Cc1ccccc1)C3)[C@H](CC(C)(C)C)CO2] | 1.99 | 694.319 | 695.3 | A |
| 431 | 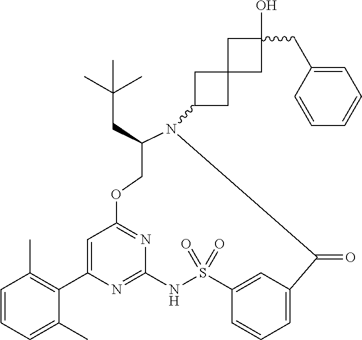 [SMILES: Cc1cccc(C)c1-c1cc2nc(n1)NS(=O)(=O)c1cccc(c1)C(=O)N(C1CC3(C1)CC(O)(Cc1ccccc1)C3)[C@H](CC(C)(C)C)CO2] | 2.08 | 694.319 | 695.3 | A |
| 432 | 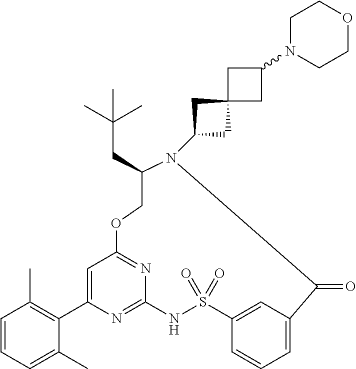 | 1.34 | 673.33 | 674.3 | A |
| 433 | 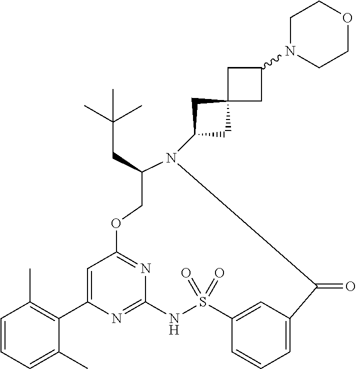 | 1.36 | 673.33 | 674.6 | A |
| 434 | 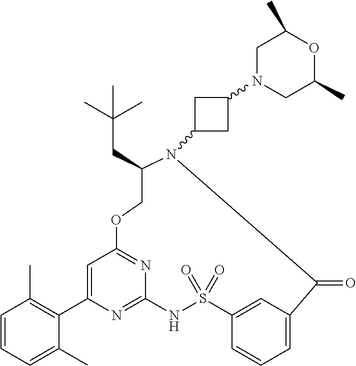 [SMILES: Cc1cccc(C)c1-c1cc2nc(n1)NS(=O)(=O)c1cccc(c1)C(=O)N(C1CC(N3C[C@@H](C)O[C@@H](C)C3)C1)[C@H](CC(C)(C)C)CO2] | 1.39 | 661.33 | 662.2 | A |
| 8 | 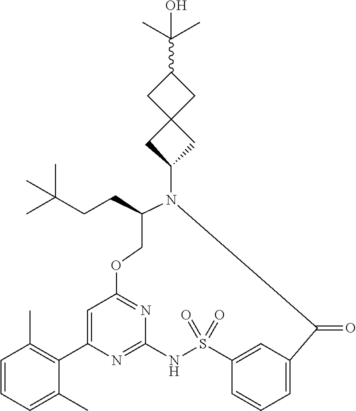 | 2.99 | 660.335 | 661.3 | I |
| 9 | 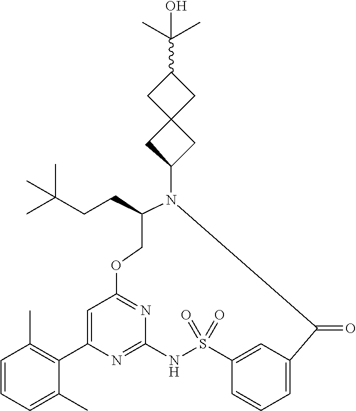 | 2.93 | 660.335 | 661.3 | I |
| 435 | 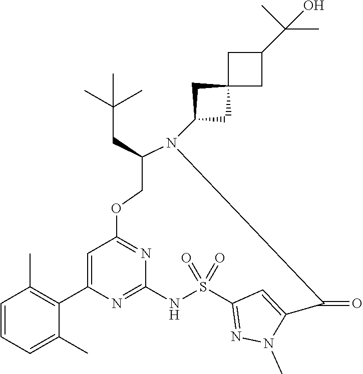 | 2.64 | 650.325 | 651.4 | A |
| 436 | 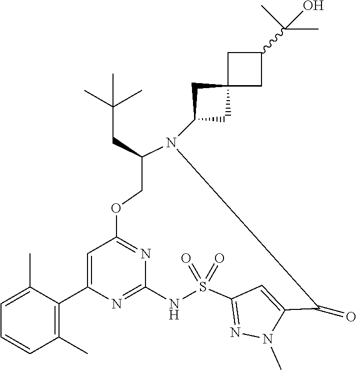 | 2.57 | 650.325 | 651.5 | I |
| 437 | 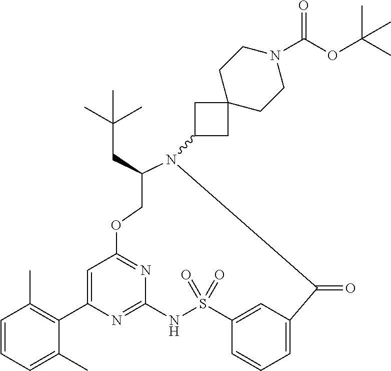 [SMILES: Cc1cccc(C)c1-c1cc2nc(n1)NS(=O)(=O)c1cccc(c1)C(=O)N(C1CC3(CCN(C(=O)OC(C)(C)C)CC3)C1)[C@H](CC(C)(C)C)CO2] | 2.27 | 717.356 | 718.5 | A |
| 438 | 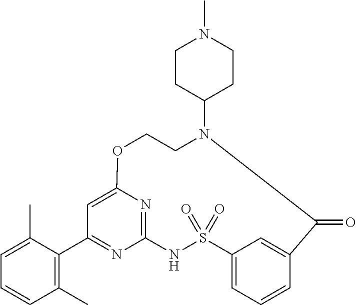 [SMILES: Cc1cccc(C)c1-c1cc2nc(n1)NS(=O)(=O)c1cccc(c1)C(=O)N(C1CCN(C)CC1)CCO2] | 0.87 | 521.21 | 522.3 | A |
| 439 | 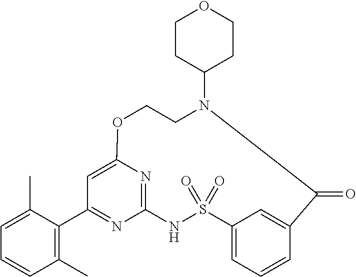 [SMILES: Cc1cccc(C)c1-c1cc2nc(n1)NS(=O)(=O)c1cccc(c1)C(=O)N(C1CCOCC1)CCO2] | 1.27 | 508.178 | 509.3 | A |
| 440 | 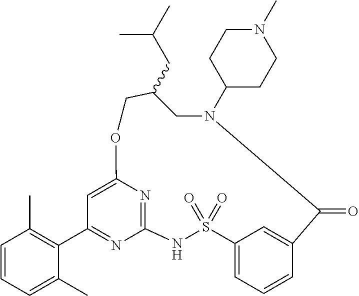 [SMILES: Cc1cccc(C)c1-c1cc2nc(n1)NS(=O)(=O)c1cccc(c1)C(=O)N(C1CCN(C)CC1)CC(CC(C)C)CO2] | 1.22 | 591.288 | 592.1 | A |
| 441 | 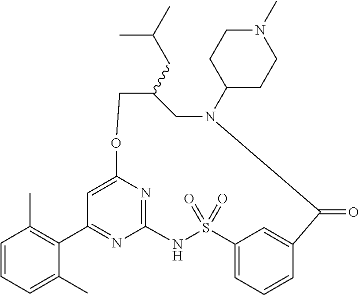 [SMILES: Cc1cccc(C)c1-c1cc2nc(n1)NS(=O)(=O)c1cccc(c1)C(=O)N(C1CCN(C)CC1)CC(CC(C)C)CO2] | 1.22 | 591.288 | 592.1 | A |
| 109 | 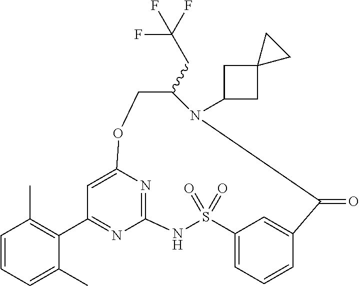 [SMILES: Cc1cccc(C)c1-c1cc2nc(n1)NS(=O)(=O)c1cccc(c1)C(=O)N(C1CC3(CC3)C1)C(CC(F)(F)F)CO2] | 1.82 | 586.186 | 587.3 | A |
| 125 | 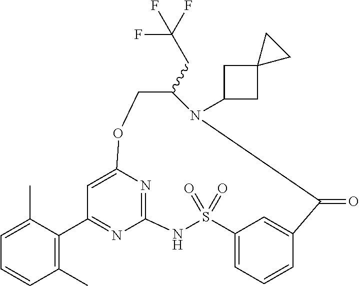 [SMILES: Cc1cccc(C)c1-c1cc2nc(n1)NS(=O)(=O)c1cccc(c1)C(=O)N(C1CC3(CC3)C1)C(CC(F)(F)F)CO2] | 1.82 | 586.186 | 587.3 | A |
| 126 | 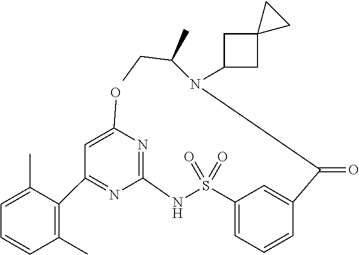 [SMILES: CC[C@@H](C)N(C(=O)c1cccc(S(=O)(=O)Nc2nc(O)cc(-c3c(C)cccc3C)n2)c1)C1CC2(CC2)C1] | 1.77 | 518.199 | 519.3 | A |
| 442 | 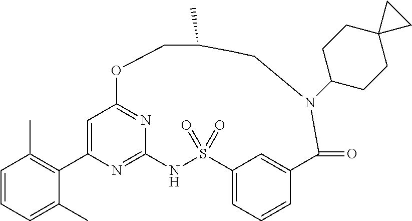 [SMILES: Cc1cccc(C)c1-c1cc2nc(n1)NS(=O)(=O)c1cccc(c1)C(=O)N(C1CCC3(CC1)CC3)CC[C@@H](C)CO2] | 1.94 | 546.23 | 547.3 | A |
| 77 | 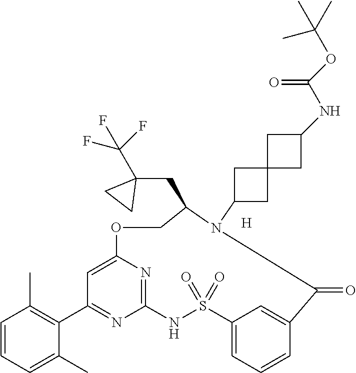 | 2.89 | 755.296 | 756.2 | I |
| 89 | 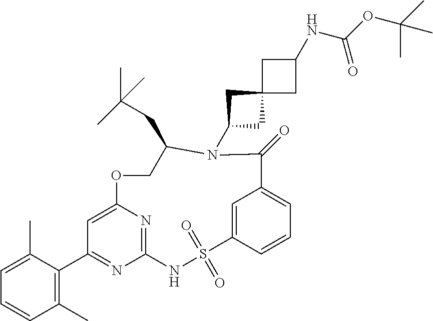 | 2.96 | 703.34 | 704.4 | I |
| 90 | 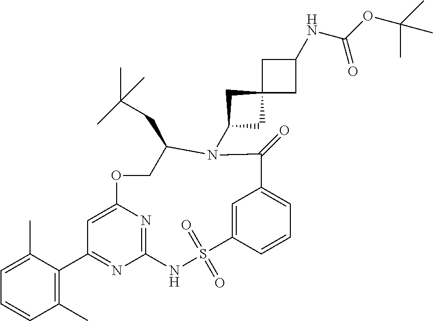 | 2.99 | 703.34 | 704.3 | I |
| 443 | 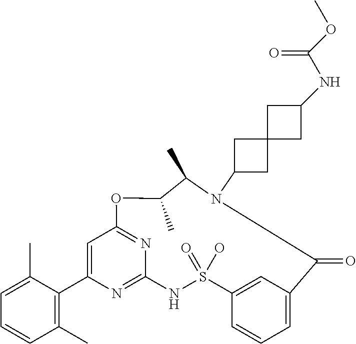 | 1.6 | 619.246 | 620.5 | A |
| 444 | 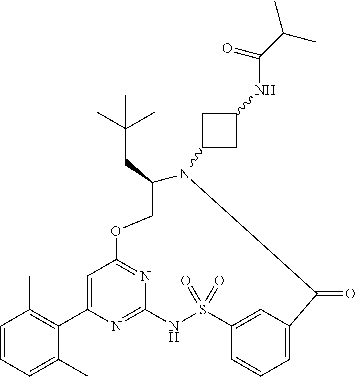 [SMILES: Cc1cccc(C)c1-c1cc2nc(n1)NS(=O)(=O)c1cccc(c1)C(=O)N(C1CC(NC(=O)C(C)C)C1)[C@H](CC(C)(C)C)CO2] | 1.7 | 633.298 | 634.3 | A |
| 445 | 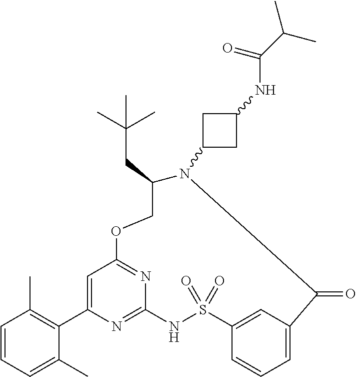 [SMILES: Cc1cccc(C)c1-c1cc2nc(n1)NS(=O)(=O)c1cccc(c1)C(=O)N(C1CC(NC(=O)C(C)C)C1)[C@H](CC(C)(C)C)CO2] | 1.68 | 633.298 | 634.2 | A |
| 446 | 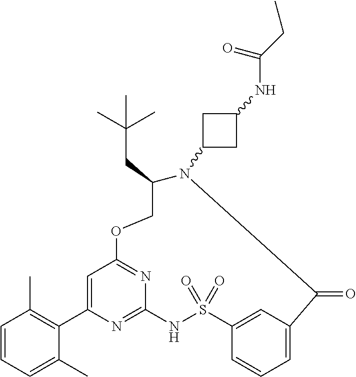 [SMILES: CCC(=O)NC1CC(N2C(=O)c3cccc(c3)S(=O)(=O)Nc3nc(cc(-c4c(C)cccc4C)n3)OC[C@H]2CC(C)(C)C)C1] | 1.62 | 619.283 | 620.7 | A |
| 447 | 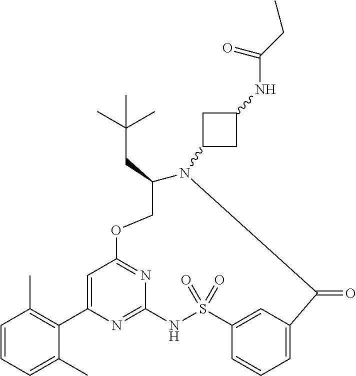 [SMILES: CCC(=O)NC1CC(N2C(=O)c3cccc(c3)S(=O)(=O)Nc3nc(cc(-c4c(C)cccc4C)n3)OC[C@H]2CC(C)(C)C)C1] | 1.59 | 619.283 | 620.5 | A |
| 448 | 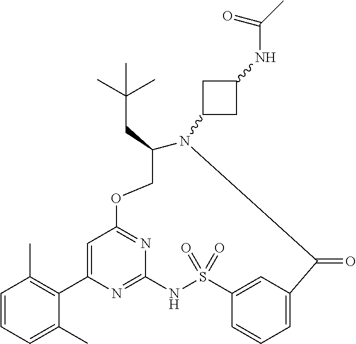 [SMILES: CC(=O)NC1CC(N2C(=O)c3cccc(c3)S(=O)(=O)Nc3nc(cc(-c4c(C)cccc4C)n3)OC[C@H]2CC(C)(C)C)C1] | 1.53 | 605.267 | 606.2 | A |
| 449 | 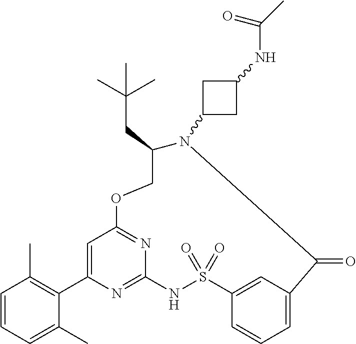 [SMILES: CC(=O)NC1CC(N2C(=O)c3cccc(c3)S(=O)(=O)Nc3nc(cc(-c4c(C)cccc4C)n3)OC[C@H]2CC(C)(C)C)C1] | 1.5 | 605.267 | 606.3 | A |
| 450 | 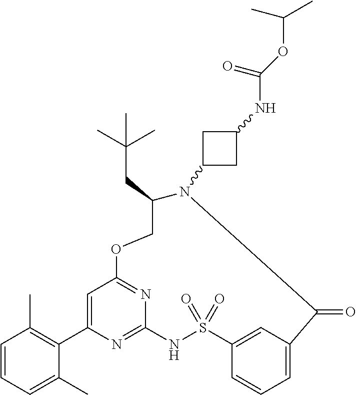 [SMILES: Cc1cccc(C)c1-c1cc2nc(n1)NS(=O)(=O)c1cccc(c1)C(=O)N(C1CC(NC(=O)OC(C)C)C1)[C@H](CC(C)(C)C)CO2] | 1.89 | 649.293 | 650.6 | A |
| 451 | 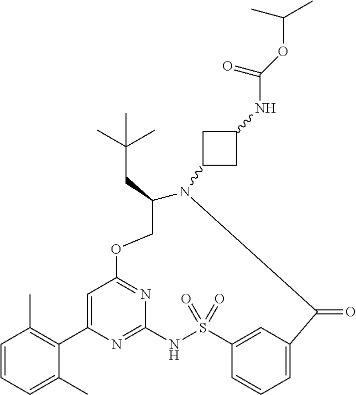 [SMILES: Cc1cccc(C)c1-c1cc2nc(n1)NS(=O)(=O)c1cccc(c1)C(=O)N(C1CC(NC(=O)OC(C)C)C1)[C@H](CC(C)(C)C)CO2] | 1.84 | 649.293 | 650.6 | A |
| 452 | 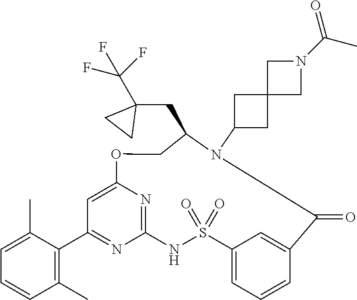 [SMILES: CC(=O)N1CC2(CC(N3C(=O)c4cccc(c4)S(=O)(=O)Nc4nc(cc(-c5c(C)cccc5C)n4)OC[C@H]3CC3(C(F)(F)F)CC3)C2)C1] | 1.51 | 683.239 | 684.5 | A |
| 453 | 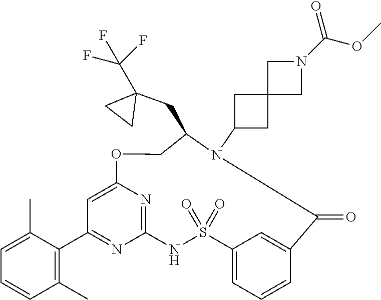 [SMILES: COC(=O)N1CC2(CC(N3C(=O)c4cccc(c4)S(=O)(=O)Nc4nc(cc(-c5c(C)cccc5C)n4)OC[C@H]3CC3(C(F)(F)F)CC3)C2)C1] | 1.68 | 699.234 | 700.6 | A |
| 454 | 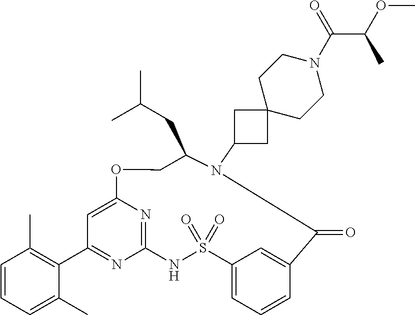 [SMILES: CO[C@@H](C)C(=O)N1CCC2(CC1)CC(N1C(=O)c3cccc(c3)S(=O)(=O)Nc3nc(cc(-c4c(C)cccc4C)n3)OC[C@H]1CC(C)C)C2] | 1.73 | 689.325 | 690.5 | A |
| 455 | 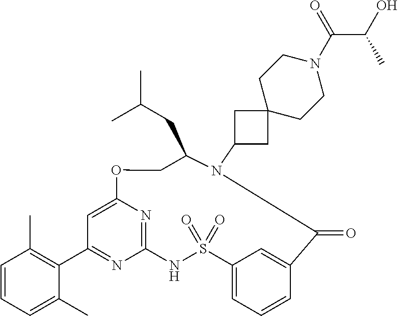 [SMILES: Cc1cccc(C)c1-c1cc2nc(n1)NS(=O)(=O)c1cccc(c1)C(=O)N(C1CC3(CCN(C(=O)[C@@H](C)O)CC3)C1)[C@H](CC(C)C)CO2] | 1.69 | 675.309 | 676.3 | A |
| 456 | 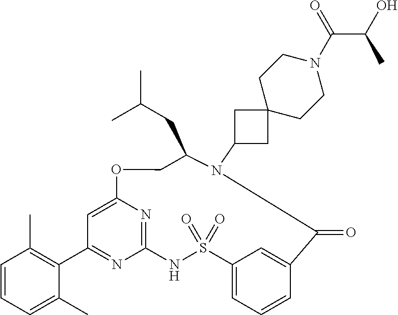 [SMILES: Cc1cccc(C)c1-c1cc2nc(n1)NS(=O)(=O)c1cccc(c1)C(=O)N(C1CC3(CCN(C(=O)[C@H](C)O)CC3)C1)[C@H](CC(C)C)CO2] | 1.66 | 675.309 | 676.5 | A |
| 457 | 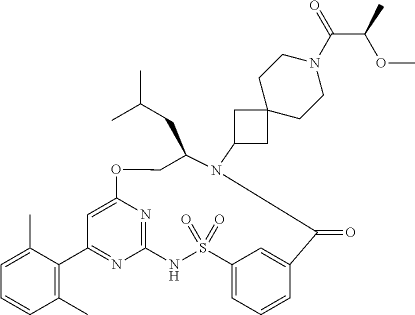 [SMILES: CO[C@H](C)C(=O)N1CCC2(CC1)CC(N1C(=O)c3cccc(c3)S(=O)(=O)Nc3nc(cc(-c4c(C)cccc4C)n3)OC[C@H]1CC(C)C)C2] | 1.74 | 689.325 | 690.3 | A |
| 458 | 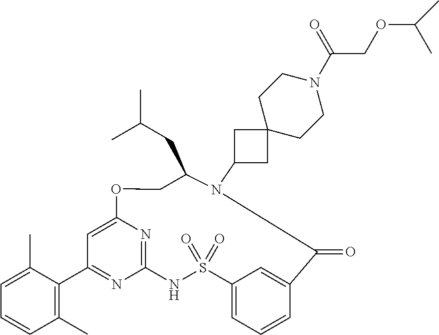 [SMILES: Cc1cccc(C)c1-c1cc2nc(n1)NS(=O)(=O)c1cccc(c1)C(=O)N(C1CC3(CCN(C(=O)COC(C)C)CC3)C1)[C@H](CC(C)C)CO2] | 1.95 | 703.34 | 704.3 | A |
| 459 | 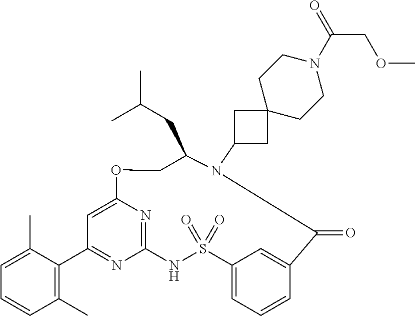 [SMILES: COCC(=O)N1CCC2(CC1)CC(N1C(=O)c3cccc(c3)S(=O)(=O)Nc3nc(cc(-c4c(C)cccc4C)n3)OC[C@H]1CC(C)C)C2] | 1.76 | 675.309 | 676.3 | A |
| 460 | 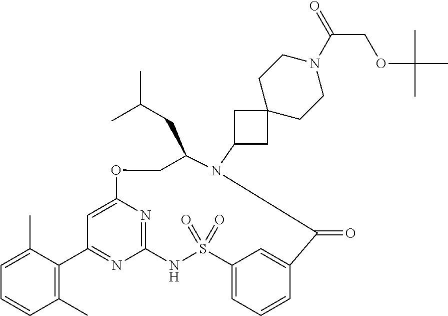 [SMILES: Cc1cccc(C)c1-c1cc2nc(n1)NS(=O)(=O)c1cccc(c1)C(=O)N(C1CC3(CCN(C(=O)COC(C)(C)C)CC3)C1)[C@H](CC(C)C)CO2] | 2.01 | 717.356 | 718.3 | A |
| 461 | 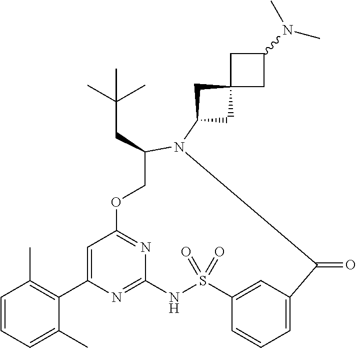 | 1.32 | 631.319 | 632.7 | A |
| 462 | 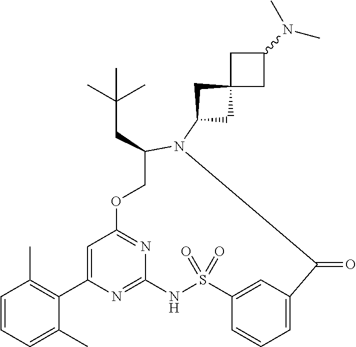 | 1.34 | 631.319 | 632.7 | 1A |
| 463 | 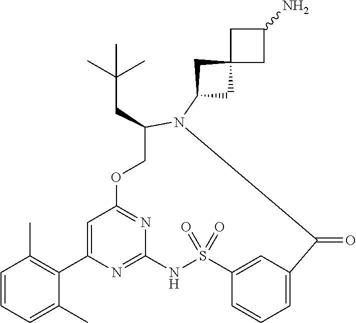 | 1.3 | 603.288 | 604.55 | A |
| 118 | 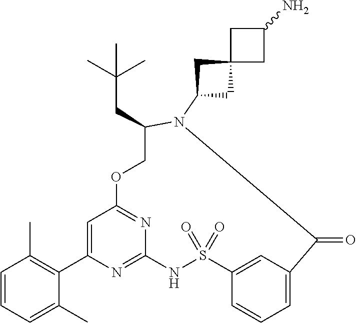 | 1.32 | 603.288 | 604.5 | A |
| 47 | 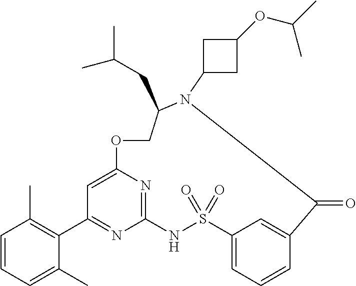 [SMILES: Cc1cccc(C)c1-c1cc2nc(n1)NS(=O)(=O)c1cccc(c1)C(=O)N(C1CC(OC(C)C)C1)[C@H](CC(C)C)CO2] | 1.98 | 592.272 | 593.2 | A |
| 464 | 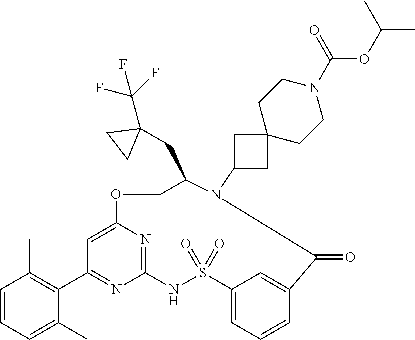 [SMILES: Cc1cccc(C)c1-c1cc2nc(n1)NS(=O)(=O)c1cccc(c1)C(=O)N(C1CC3(CCN(C(=O)OC(C)C)CC3)C1)[C@H](CC1(C(F)(F)F)CC1)CO2] | 1.99 | 755.296 | 756.5 | A |
| 465 | 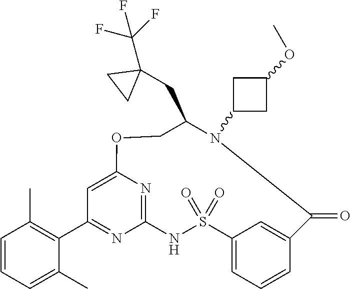 [SMILES: COC1CC(N2C(=O)c3cccc(c3)S(=O)(=O)Nc3nc(cc(-c4c(C)cccc4C)n3)OC[C@H]2CC2(C(F)(F)F)CC2)C1] | 1.68 | 630.212 | 631.4 | A |
| 466 | 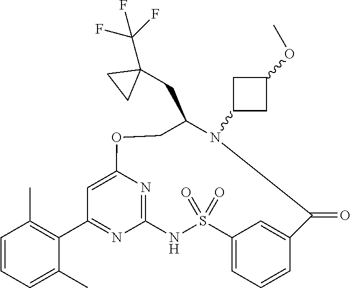 [SMILES: COC1CC(N2C(=O)c3cccc(c3)S(=O)(=O)Nc3nc(cc(-c4c(C)cccc4C)n3)OC[C@H]2CC2(C(F)(F)F)CC2)C1] | 1.67 | 630.212 | 631.5 | A |
| 467 | 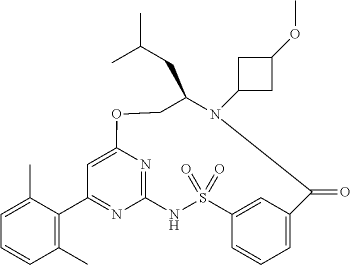 [SMILES: COC1CC(N2C(=O)c3cccc(c3)S(=O)(=O)Nc3nc(cc(-c4c(C)cccc4C)n3)OC[C@H]2CC(C)C)C1] | 1.72 | 564.241 | 565.3 | A |
| 468 | 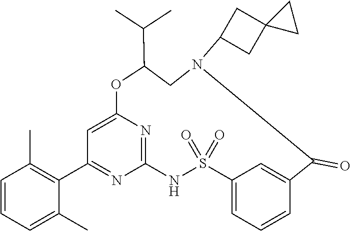 [SMILES: Cc1cccc(C)c1-c1cc2nc(n1)NS(=O)(=O)c1cccc(c1)C(=O)N(C1CC3(CC3)C1)CC(C(C)C)O2] | 1.94 | 546.23 | 547.3 | A |
| 469 | 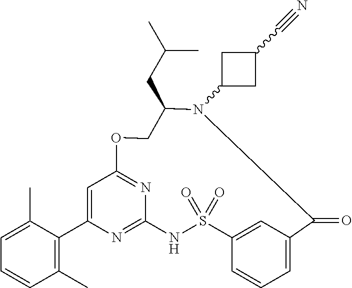 | 1.65 | 559.225 | 560.3 | A |
| 470 | 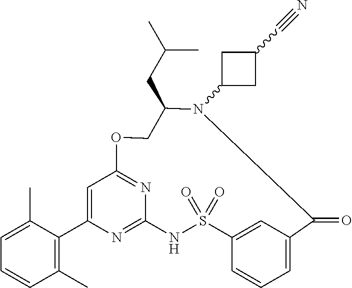 | 1.68 | 559.225 | 560.2 | A |
| 471 | 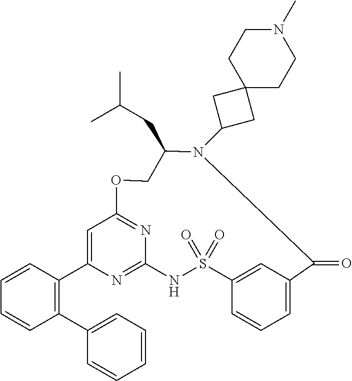 | 1.43 | 665.304 | 666.6 | A |
| 12 | 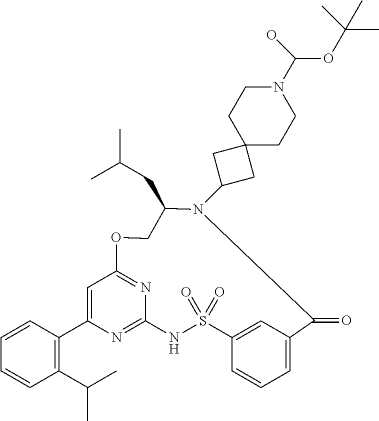 | 0.84 | 717.356 | 718.4 | D |
| 473 | 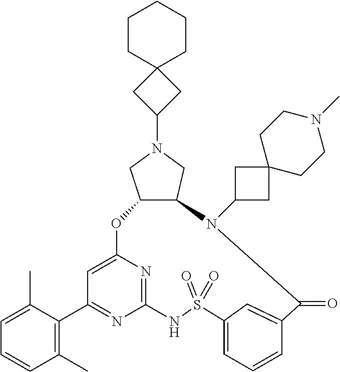 [SMILES: Cc1cccc(C)c1-c1cc2nc(n1)NS(=O)(=O)c1cccc(c1)C(=O)N(C1CC3(CCN(C)CC3)C1)[C@@H]1CN(C3CC4(CCCCC4)C3)C[C@H]1O2] | 1.16 | 724.377 | 725.7 | A |

TABLE 6
NMR data of new compounds

| Compound | |
|---|---|
| number | NMR |
| 199 | ¹H NMR (400 MHz, Methanol-d₄) δ 8.58 (d, J = 6.2 Hz, 1H), 8.04 (d, J = |
| | 7.4 Hz, 1H), 7.71 (p, J = 7.5 Hz, 2H), 7.29 (t, J = 7.6 Hz, 1H), 7.16 (d, |
| | J = 7.6 Hz, 2H), 6.29 (s, 1H), 5.30 (ddd, J = 11.3, 7.3, 4.3 Hz, 1H), 4.27 |
| | (q, J = 10.9 Hz, 1H), 4.12 (q, J = 7.1 Hz, 2H), 3.98 (p, J = 8.7 Hz, 2H), |
| | 3.84 (p, J = 5.4 Hz, 1H), 3.19 (dd, J = 25.7, 9.9 Hz, 1H), 3.10 (q, J = 8.6 |
| | Hz, 1H), 2.54 (dq, J = 13.3, 7.2 Hz, 1H), 2.48-2.37 (m, 2H), 2.26 (s, |
| | 1H), 2.03 (s, 4H), 2.01 (s, 2H), 1.71 (dt, J = 15.1, 7.5 Hz, 1H), 1.54 (d, J = |
| | 15.0 Hz, 1H), 1.45 (s, 8H), 1.26 (t, J = 7.1 Hz, 3H), 0.60 (d, J = 3.9 |
| | Hz, 9H). |
| 209 | ¹H NMR (500 MHz, DMSO-d₆) δ 13.02 (broad s, 1H), 8.39 (broad s, 1H), |
| | 7.90 (broad s, 1H), 7.65 (broad s, 2H), 7.33 (d, J = 3.0 Hz, 2H), 7.25 (d, |
| | J = 8.0 Hz, 1H), 7.17 (s, 1H), 6.44 (broad s, 1H), 5.07-5.01 (m, 1H), 4.70 |
| | (p, J = 6.3 Hz, 1H), 4.36-4.19 (m, 2H), 4.05 (s, 1H), 3.95-3.79 (m, 2H), |
| | 3.70-3.58 (m, 1H), 3.04 (t, J = 9.5 Hz, 1H), 2.95 (t, J = 9.9 Hz, 1H), |
| | 2.42-2.29 (m, 2H), 2.26-2.20 (m, 1H), 2.19-2.05 (m, 2H), 2.01-1.82 |
| | (m, 4H), 1.65-1.52 (m, 1H), 1.34 (d, J = 15.0 Hz, 1H), 1.13 (d, J = 6.3 |
| | Hz, 6H), 0.48 (s, 9H). |
| 210 | ¹H NMR (500 MHz, DMSO-d₆) δ 8.47 (s, 1H), 7.93 (d, J = 7.6 Hz, 1H), |
| | 7.78-7.60 (m, 2H), 7.27 (t, J = 7.6 Hz, 1H), 7.13 (d, J = 7.6 Hz, 2H), |
| | 6.40 (s, 1H), 5.20 (dd, J = 10.8, 4.2 Hz, 1H), 4.72 (p, J = 7.2 Hz, 1H), |
| | 4.25 (t, J = 11.2 Hz, 1H), 3.72 (q, J = 8.0 Hz, 2H), 3.35 (dq, J = 13.7, |
| | 5.3, 3.8 Hz, 2H), 2.67 (ddt, J = 16.0, 12.6, 7.8 Hz, 2H), 1.98 (s, 6H), 1.62 |
| | (ddd, J = 14.1, 10.8, 2.8 Hz, 1H), 1.28 (dtd, J = 15.6, 8.5, 7.5, 4.4 Hz, |
| | 1H), 1.16 (ddd, J = 13.7, 10.5, 2.8 Hz, 1H), 0.75 (d, J = 6.6 Hz, 3H), 0.22 |
| | (d, J = 6.3 Hz, 3H). |
| 211 | ¹H NMR (500 MHz, DMSO-d₆) δ 8.46 (s, 1H), 7.93 (d, J = 7.4 Hz, 1H), |
| | 7.80-7.63 (m, 2H), 7.27 (t, J = 7.7 Hz, 1H), 7.13 (d, J = 7.6 Hz, 2H), |
| | 6.40 (s, 1H), 5.47 (tt, J = 8.2, 4.3 Hz, 1H), 5.10 (dd, J = 10.7, 4.4 Hz, |
| | 1H), 4.40 (tt, J = 10.6, 5.8 Hz, 1H), 4.30 (t, J = 11.1 Hz, 1H), 3.83-3.72 |
| | (m, 1H, overlapping with water peak), 3.20 (dp, J = 36.7, 6.5 Hz, 2H), |
| | 2.74-2.56 (m, 2H), 2.03 (d, J = 61.7 Hz, 6H), 1.54 (dd, J = 14.2, 10.7 |
| | Hz, 1H), 1.32-1.20 (m, 1H), 1.18-1.08 (m, 1H), 0.71 (d, J = 6.6 Hz, |
| | 3H), 0.18 (d, J = 6.3 Hz, 3H). |
| 212 | ¹H NMR (500 MHz, DMSO-d₆) δ 13.07 (s, 1H), 8.42 (s, 1H), 7.91 (s, |
| | 1H), 7.68 (s, 2H), 7.27 (s, 1H), 7.13 (s, 2H), 6.40 (s, 1H), 5.21 (tt, J = 7.5, |
| | 3.5 Hz, 1H), 5.06 (dd, J = 10.9, 4.6 Hz, 1H), 4.31 (qd, J = 9.7, 6.6 Hz, |
| | 2H), 4.07 (s, 1H), 3.25 (dq, J = 14.5, 7.4 Hz, 2H), 2.61 (ddd, J = 22.7, |
| | 13.0, 9.6 Hz, 2H), 2.34-1.72 (m, 7H), 1.52 (dd, J = 16.6, 8.9 Hz, 1H), |
| | 0.82 (dt, J = 10.6, 5.3 Hz, 1H), 0.74 (dt, J = 10.2, 5.0 Hz, 1H), 0.66- |
| | 0.48 (m, 2H). |
| 213 | ¹H NMR (500 MHz, DMSO-d₆) δ 8.47 (s, 1H), 7.91 (dd, J = 15.0, 7.7 |
| | Hz, 1H), 7.78-7.61 (m, 2H), 7.27 (t, J = 7.6 Hz, 1H), 7.13 (d, J = 7.7 |
| | Hz, 2H), 6.40 (s, 1H), 5.19 (dd, J = 10.8, 4.2 Hz, 1H), 4.57 (p, J = 7.2 |
| | Hz, 1H), 4.25 (t, J = 11.3 Hz, 1H), 3.71 (p, J = 9.3, 8.7 Hz, 2H), 3.37- |
| | 3.26 (m, 2H), 2.65 (dddd, J = 20.5, 12.6, 8.0, 5.4 Hz, 2H), 1.97 (s, 6H), |
| | 1.62 (ddd, J = 14.0, 10.8, 2.8 Hz, 1H), 1.28 (dtq, J = 13.9, 7.4, 3.5 Hz, |
| | 1H), 1.16 (ddd, J = 13.7, 10.4, 2.8 Hz, 1H), 0.75 (d, J = 6.6 Hz, 3H), |
| | 0.27-0.15 (m, 3H). |
| 214 | ¹H NMR (500 MHz, DMSO-d₆) δ 8.45 (s, 1H), 7.93 (d, J = 6.3 Hz, 1H), |
| | 7.70 (d, J = 17.6 Hz, 2H), 7.27 (t, J = 7.6 Hz, 1H), 7.13 (d, J = 7.6 Hz, |
| | 2H), 6.40 (s, 1H), 5.28 (tt, J = 7.9, 4.0 Hz, 1H), 5.11 (dd, J = 10.7, 4.4 |
| | Hz, 1H), 4.43-4.25 (m, 2H), 3.81-3.71 (m, 1H), 3.20 (ddt, J = 32.2, |
| | 13.8, 7.0 Hz, 2H), 2.62 (dt, J = 30.8, 12.7 Hz, 2H), 2.00 (d, J = 43.8 Hz, |
| | 6H), 1.55 (t, J = 11.4 Hz, 1H), 1.26 (dtd, J = 15.7, 8.7, 7.6, 4.5 Hz, 1H), |
| | 1.14 (t, J = 13.4 Hz, 1H), 0.71 (d, J = 6.6 Hz, 3H), 0.19 (d, J = 6.2 Hz, |
| | 3H). |
| 216 | ¹H NMR (400 MHz, Chloroform-d) δ 8.65 (t, J = 1.8 Hz, 1H), 7.94 (dt, J = |
| | 7.8, 1.5 Hz, 1H), 7.85 (dt, J = 7.7, 1.3 Hz, 1H), 7.62 (t, J = 7.8 Hz, 1H), |
| | 7.22 (t, J = 7.6 Hz, 1H), 7.07 (d, J = 7.6 Hz, 2H), 6.25 (s, 1H), 5.33 (dd, |
| | J = 9.9, 3.4 Hz, 1H), 4.15-4.00 (m, 3H), 3.44-3.35 (m, 1H), 3.29 (dd, |
| | J = 10.5, 8.7 Hz, 1H), 2.22 (t, J = 10.0 Hz, 1H), 2.14 (t, J = 9.6 Hz, 1H), |
| | 2.01 (s, 6H), 1.94-1.84 (m, 2H), 0.90 (s, 3H), 0.67 (s, 3H), 0.61-0.49 |
| | (m, 4H). |
| 217 | ¹H NMR (400 MHz, Chloroform-d) δ 8.90 (s, 1H), 8.65 (s, 1H), 7.93 (d, |
| | J = 7.9 Hz, 1H), 7.85 (d, J = 7.5 Hz, 1H), 7.62 (t, J = 7.8 Hz, 1H), 7.22 |
| | (t, J = 7.6 Hz, 1H), 7.07 (d, J = 7.6 Hz, 2H), 6.25 (s, 1H), 5.40-5.29 (m, |
| | 1H), 4.15-3.98 (m, 3H), 3.39 (t, J = 9.5 Hz, 1H), 3.29 (t, J = 9.5 Hz, |
| | 1H), 2.23 (d, J = 9.5 Hz, 1H), 2.14 (t, J = 9.7 Hz, 1H), 2.01 (s, 6H), 1.93- |
| | 1.81 (m, 2H), 0.90 (s, 3H), 0.67 (s, 3H), 0.55 (d, J = 4.3 Hz, 4H). |
| 259 | ¹H NMR (400 MHz, DMSO-d₆) δ 13.04 (s, 1H), 8.34 (s, 1H), 7.88 (s, |
| | 1H), 7.65 (s, 2H), 7.25 (s, 1H), 7.12 (s, 2H), 6.37 (s, 1H), 5.05 (dd, J = |
| | 11.0, 4.4 Hz, 1H), 4.35 (s, 2H), 4.06 (s, 1H), 3.80 (p, J = 8.9 Hz, 1H), |
| | 3.57 (s, 3H), 2.98 (dt, J = 27.6, 9.7 Hz, 2H), 2.76 (s, 3H), 2.32 (s, 2H), |
| | 2.29-1.70 (m, 11H), 1.49 (dd, J = 16.6, 9.4 Hz, 1H), 0.87-0.70 (m, 2H), |
| | 0.59 (d, J = 29.5 Hz, 2H). |
| 260 | ¹H NMR (400 MHz, DMSO-d₆) δ 13.04 (s, 1H), 8.32 (s, 1H), 7.87 (s, |
| | 1H), 7.65 (s, 2H), 7.25 (s, 1H), 7.12 (s, 2H), 6.37 (s, 1H), 5.03 (dd, J = |
| | 10.7, 4.3 Hz, 1H), 4.33 (s, 2H), 4.05 (s, 1H), 3.79 (q, J = 8.9 Hz, 1H), |
| | 3.57 (s, 3H), 3.06 (t, J = 9.5 Hz, 1H), 2.97 (t, J = 10.0 Hz, 1H), 2.77 (s, |
| | 3H), 2.29 (s, 2H), 2.25-1.78 (m, 11H), 1.50 (dd, J = 16.5, 9.4 Hz, 1H), |
| | 0.79 (d, J = 11.3 Hz, 2H), 0.62 (d, J = 28.5 Hz, 2H). |
| 266 | ¹H NMR (400 MHz, DMSO-d₆) δ 13.04 (s, 1H), 9.02-8.66 (m, 2H), 8.34 |
| | (s, 1H), 7.89 (d, J = 7.1 Hz, 1H), 7.66 (s, 2H), 7.26 (t, J = 7.5 Hz, 1H), |
| | 7.12 (d, J = 7.7 Hz, 2H), 6.38 (s, 1H), 5.05 (dd, J = 10.8, 4.4 Hz, 1H), |
| | 4.32 (t, J = 11.2 Hz, 1H), 4.06 (d, J = 11.7 Hz, 1H), 3.83 (p, J = 8.7 Hz, |
| | 1H), 3.74-3.61 (m, 1H), 3.18 (dt, J = 12.7, 6.3 Hz, 1H), 3.06 (t, J = 9.5 |
| | Hz, 1H), 2.97 (t, J = 10.0 Hz, 1H), 2.49-2.40 (m, 1H), 2.30 (qt, J = 11.2, |
| | 8.6, 4.0 Hz, 4H), 2.19 (dd, J = 20.1, 13.2 Hz, 3H), 1.92 (s, 4H), 1.51 (dd, |
| | J = 16.5, 9.5 Hz, 1H), 1.19 (d, J = 6.5 Hz, 6H), 0.81 (ddt, J = 19.4, 10.6, |
| | 5.6 Hz, 2H), 0.66 (dd, J = 10.3, 5.2 Hz, 1H), 0.52 (s, 1H). |
| 267 | ¹H NMR (400 MHz, DMSO-d₆) δ 13.04 (s, 1H), 8.75 (s, 2H), 8.34 (s, |
| | 1H), 7.88 (s, 1H), 7.66 (s, 2H), 7.26 (t, J = 7.7 Hz, 1H), 7.12 (d, J = 7.4 |
| | Hz, 2H), 6.39 (s, 1H), 5.05 (dd, J = 10.8, 4.5 Hz, 1H), 4.33 (t, J = 11.3 |
| | Hz, 1H), 4.06 (d, J = 10.3 Hz, 1H), 3.83 (p, J = 8.6 Hz, 1H), 3.54 (t, J = |
| | 7.4 Hz, 1H), 3.00 (dt, J = 36.5, 9.7 Hz, 2H), 2.43 (t, J = 4.1 Hz, 3H), 2.32 |
| | (d, J = 18.0 Hz, 2H), 2.25-2.11 (m, 5H), 2.07-1.83 (m, 4H), 1.49 (dd, |
| | J = 16.5, 9.4 Hz, 1H), 1.27-1.22 (m, 1H), 0.84-0.72 (m, 2H), 0.68- |
| | 0.48 (m, 2H). |
| 268 | ¹H NMR (400 MHz, DMSO-d₆) δ 13.05 (s, 1H), 8.72 (s, 2H), 8.33 (s, |
| | 1H), 7.89 (s, 1H), 7.66 (s, 2H), 7.26 (t, J = 7.5 Hz, 1H), 7.13 (s, 2H), 6.38 |
| | (s, 1H), 5.05 (dd, J = 10.8, 4.4 Hz, 1H), 4.31 (t, J = 11.3 Hz, 1H), 4.04 |
| | (s, 1H), 3.83 (p, J = 8.6 Hz, 1H), 3.54 (q, J = 7.4 Hz, 1H), 3.05 (t, J = 9.6 |
| | Hz, 1H), 2.97 (t, J = 10.0 Hz, 1H), 2.43 (t, J = 5.5 Hz, 3H), 2.29 (t, J = |
| | 6.4 Hz, 2H), 2.25-2.12 (m, 4H), 1.91 (s, 5H), 1.51 (dd, J = 16.5, 9.3 Hz, |
| | 1H), 1.29-1.21 (m, 1H), 0.84-0.72 (m, 2H), 0.70-0.45 (m, 2H). |
| 269 | ¹H NMR (400 MHz, DMSO-d₆) δ 13.02 (s, 1H), 8.82 (d, J = 52.9 Hz, |
| | 2H), 8.34 (s, 1H), 7.89 (d, J = 7.0 Hz, 1H), 7.66 (s, 2H), 7.26 (t, J = 7.6 |
| | Hz, 1H), 7.12 (d, J = 7.7 Hz, 2H), 6.39 (s, 1H), 5.05 (dd, J = 10.9, 4.5 |
| | Hz, 1H), 4.33 (t, J = 11.4 Hz, 1H), 4.05 (s, 1H), 3.82 (q, J = 8.5 Hz, 1H), |
| | 3.67-3.59 (m, 1H), 3.54 (t, J = 5.0 Hz, 3H), 3.04 (t, J = 9.9 Hz, 1H), |
| | 3.02-2.90 (m, 3H), 2.45 (dd, J = 11.8, 7.0 Hz, 1H), 2.35-1.83 (m, 12H), |
| | 1.49 (dd, J = 16.5, 9.3 Hz, 1H), 1.28-1.23 (m, 1H), 0.83-0.72 (m, 2H), |
| | 0.58 (d, J = 28.5 Hz, 2H). |
| 273 | ¹H NMR (400 MHz, DMSO-d₆) δ 13.01 (s, 1H), 8.33 (s, 1H), 7.87 (s, |
| | 1H), 7.64 (s, 2H), 7.36 (d, J = 7.8 Hz, 1H), 7.25 (s, 1H), 7.11 (s, 2H), 6.37 |
| | (s, 1H), 5.04 (d, J = 10.7 Hz, 1H), 4.34 (s, 1H), 4.05 (s, 1H), 3.93-3.72 |
| | (m, 2H), 3.50 (s, 3H), 2.95 (dt, J = 27.6, 9.7 Hz, 2H), 2.49-2.36 (m, 2H), |
| | 2.36-1.63 (m, 11H), 1.48 (dd, J = 16.6, 9.5 Hz, 1H), 0.77 (d, J = 12.5 |
| | Hz, 2H), 0.59 (d, J = 18.8 Hz, 2H). |
| 274 | ¹H NMR (400 MHz, DMSO-d₆) δ 13.03 (s, 1H), 8.32 (s, 1H), 7.87 (s, |
| | 1H), 7.65 (s, 2H), 7.36 (d, J = 7.9 Hz, 1H), 7.25 (d, J = 7.8 Hz, 1H), 7.11 |
| | (d, J = 7.6 Hz, 2H), 6.37 (s, 1H), 5.03 (dd, J = 10.8, 4.5 Hz, 1H), 4.32 (t, |
| | J = 11.4 Hz, 1H), 4.11-3.98 (m, 1H), 3.94-3.68 (m, 2H), 3.50 (s, 3H), |
| | 3.03 (t, J = 9.5 Hz, 1H), 2.94 (t, J = 9.9 Hz, 1H), 2.40 (dt, J = 11.6, 6.6 |
| | Hz, 1H), 2.30-2.04 (m, 6H), 2.00-1.85 (m, 5H), 1.49 (dd, J = 16.5, 9.4 |
| | Hz, 1H), 1.17 (t, J = 7.1 Hz, 1H), 0.78 (ddt, J = 19.4, 9.8, 4.7 Hz, 2H), |
| | 0.62 (d, J = 26.0 Hz, 2H). |
| 336 | ¹H NMR (400 MHz, CDCl3) δ 8.54 (s, 1H), 8.01 (dd, J = 12.8, 7.7 Hz, |
| | 1H), 7.75 (d, J = 7.5 Hz, 1H), 7.63 (t, J = 7.7 Hz, 1H), 7.22 (t, J = 7.6 |
| | Hz, 1H), 7.06 (d, J = 7.6 Hz, 2H), 6.23 (s, 1H), 5.27 (dd, J = 10.5, 4.1 |
| | Hz, 1H), 4.04 (t, J = 11.1 Hz, 1H), 3.94 (td, J = 11.0, 9.1, 5.2 Hz, 1H), |
| | 3.89-3.78 (m, 1H), 3.73 (d, J = 10.8 Hz, 2H), 3.64 (d, J = 17.7 Hz, 2H), |
| | 2.77 (d, J = 12.1 Hz, 2H), 2.64 (d, J = 12.0 Hz, 2H), 2.44 (s, 1H), 2.09 |
| | (d, J = 4.0 Hz, 3H), 2.02 (s, 6H), 1.86 (s, 1H), 1.60 (d, J = 26.6 Hz, 1H), |
| | 1.44 (s, 1H), 1.25 (t, J = 12.6 Hz, 1H), 0.82 (d, J = 6.6 Hz, 3H), 0.25 (d, |
| | J = 6.1 Hz, 3H). |
| 338 | ¹H NMR (400 MHz, DMSO) δ 13.06 (s, 1H), 8.37 (s, 1H), 7.90 (s, 1H), |
| | 7.67 (s, 2H), 7.25 (d, J = 7.6 Hz, 1H), 7.12 (d, J = 7.6 Hz, 2H), 6.38 (s, |
| | 1H), 5.08 (dd, J = 10.7, 4.2 Hz, 1H), 4.38 (t, J = 11.0 Hz, 1H), 3.96 (p, J = |
| | 8.9 Hz, 1H), 3.70 (t, J = 8.4 Hz, 1H), 2.72 (q, J = 10.0 Hz, 2H), 2.08 |
| | (s, 2H), 1.92 (dt, J = 29.0, 8.5 Hz, 5H), 1.69-1.50 (m, 5H), 1.43 (s, 2H), |
| | 1.34 (s, 5H), 1.13 (ddd, J = 13.6, 10.6, 2.7 Hz, 1H), 0.73 (d, J = 6.6 Hz, |
| | 3H), 0.20 (d, J = 6.1 Hz, 3H). |
| 339 | ¹H NMR (400 MHz, DMSO) δ 13.06 (s, 1H), 8.38 (s, 1H), 7.90 (s, 1H), |
| | 7.67 (s, 2H), 7.25 (d, J = 8.3 Hz, 1H), 7.12 (d, J = 7.6 Hz, 2H), 6.38 (s, |
| | 1H), 5.10 (dd, J = 10.5, 4.1 Hz, 1H), 4.38 (t, J = 11.1 Hz, 1H), 3.95 (p, J = |
| | 8.7 Hz, 1H), 3.70 (s, 1H), 3.02-2.80 (m, 2H), 2.26-1.86 (m, 8H), 1.77- |
| | 1.45 (m, 9H), 1.31 (d, J = 11.6 Hz, 1H), 1.23-1.07 (m, 1H), 0.73 (d, J = |
| | 6.6 Hz, 3H), 0.20 (d, J = 6.4 Hz, 3H). |
| 340 | ¹H NMR (400 MHz, DMSO) δ 13.07 (s, 1H), 9.57 (s, 1H), 8.39 (s, 1H), |
| | 7.91 (s, 1H), 7.68 (s, 2H), 7.25 (d, J = 8.6 Hz, 1H), 7.13 (s, 02), 6.39 (s, |
| | 1H), 5.10 (d, J = 9.8 Hz, 0H), 4.36 (s, 1H), 4.21-3.94 (m, 1H), 3.73 (s, |
| | 1H), 3.51-3.37 (m, 2H), 3.03 (s, 2), 2.77 (d, J = 9.5 Hz, 4H), 2.63-2.52 |
| | (m, 1H), 2.26-1.73 (m, 11H), 1.73-1.54 (m, 3H), 1.29 (s, 1H), 1.13 (s, |
| | 1H), 0.92 (s, 9H), 0.75 (dd, J = 12.1, 6.4 Hz, 3H), 0.20 (s, 3H),. |
| 341 | ¹H NMR (400 MHz, DMSO) δ 9.69-9.14 (m, 1H), 8.39 (s, 1H), 7.91 (d, |
| | J = 7.1 Hz, 1H), 7.67 (d, J = 8.2 Hz, 2H), 7.26 (t, J = 7.6 Hz, 1H), 7.13 |
| | (d, J = 7.6 Hz, 2H), 6.40 (s, 1H), 5.10 (dd, J = 10.6, 4.2 Hz, 1H), 4.36 |
| | (td, J = 10.8, 7.1 Hz, 1H), 4.07 (td, J = 8.8, 4.5 Hz, 1H), 3.97 (s, 1H), |
| | 3.73 (s, 1H), 3.57-3.40 (m, 5H), 3.30 (ddd, J = 11.0, 6.4, 2.7 Hz, 1H), |
| | 3.19 (dt, J = 12.7, 5.9 Hz, 1H), 3.13-2.83 (m, 4H), 2.79 (t, J = 9.6 Hz, |
| | 1H), 2.24-1.73 (m, 13H), 1.63 (q, J = 11.6 Hz, 1H), 1.28 (dq, J = 15.6, |
| | 7.4 Hz, 1H), 1.13 (dd, J = 13.6, 10.5 Hz, 1H), 0.75 (dd, J = 10.5, 6.5 Hz, |
| | 3H), 0.20 (dd, J = 6.3, 3.8 Hz, 3H). |
| 51 | ¹H NMR (400 MHz, DMSO-d₆, 80° C.) δ 8.44 (s, 1H), 7.95-7.82 (m, 1H), |
| | 7.73-7.56 (m, 2H), 7.29-7.17 (m, 1H), 7.10 (d, J = 7.6 Hz, 2H), 6.25 |
| | (s, 1H), 5.13 (dd, J = 10.6, 4.5 Hz, 1H), 4.34-4.15 (m, 2H), 3.95-3.82 |
| | (m, 1H), 3.75 (br. s., 1H), 3.27 (t, J = 9.3 Hz, 1H), 3.21-3.13 (m, 1H), |
| | 2.24-2.12 (m, 2H), 2.01 (s, 6H), 1.83 (dd, J = 14.9, 8.3 Hz, 1H), 1.58 (d, |
| | J = 13.9 Hz, 1H), 0.77 (s, 3H), 0.68 (s, 3H), 0.56-0.44 (m, 4H). |
| 52 | ¹H NMR (400 MHz, DMSO-d₆) δ 13.01 (br. s., 1H), 8.38 (br. s., 1H), 7.88 |
| | (br. s., 1H), 7.67 (br. s., 2H), 7.34-7.19 (m, 1H), 7.19-7.06 (m, 2H), |
| | 6.37 (br. s., 1H), 5.11 (dd, J = 11.1, 3.8 Hz, 1H), 4.38 (t, J = 11.1 Hz, |
| | 1H), 4.22 (quin, J = 8.4 Hz, 1H), 4.04 (s, 1H), 3.69-3.58 (m, 1H), 3.31- |
| | 3.17 (m, 2H), 2.25-1.86 (m, 8H), 1.75-1.49 (m, 2H), 1.33-1.17 (m, |
| | 1H), 0.90 (s, 6H), 0.84-0.73 (m, 1H), 0.56-0.40 (m, 4H). |
| 136 | ¹H NMR (400 MHz, DMSO-d₆) δ 8.32 (s, 1H), 7.80 (t, J = 4.9 Hz, 1H), |
| | 7.54 (d, J = 4.8 Hz, 2H), 7.18 (t, J = 7.6 Hz, 1H), 7.06 (d, J = 7.6 Hz, |
| | 2H), 6.03 (s, 1H), 5.04-4.96 (m, 1H), 4.12 (t, J = 15.2 Hz, 2H), 3.91 (t, |
| | J = 8.8 Hz, 1H), 2.84 (s, 1H), 2.74-2.65 (m, 2H), 2.55 (s, 1H), 2.37- |
| | 2.28 (m, 3H), 2.11 (d, J = 16.1 Hz, 1H), 1.97 (s, 8H), 1.72 (s, 4H), 1.52 |
| | (dd, J = 16.9, 8.6 Hz, 2H), 0.88-0.82 (m, 1H), 0.79 (dt, J = 10.8, 5.1 Hz, |
| | 1H), 0.70 (dt, J = 9.4, 4.7 Hz, 1H), 0.65 (d, J = 8.8 Hz, 1H), 0.41 (s, 1H). |
| 137 | ¹H NMR (400 MHz, DMSO-d₆) δ 8.32 (s, 1H), 7.79 (t, J = 4.7 Hz, 1H), |
| | 7.53 (d, J = 4.8 Hz, 2H), 7.17 (t, J = 7.5 Hz, 1H), 7.06 (d, J = 7.6 Hz, |
| | 2H), 6.01 (s, 1H), 5.06-4.97 (m, 1H), 4.12 (t, J = 12.3 Hz, 2H), 3.97- |
| | 3.83 (m, 1H), 2.84 (s, 1H), 2.76-2.65 (m, 2H), 2.55 (s, 1H), 2.31 (d, J = |
| | 9.5 Hz, 3H), 2.11 (d, J = 16.2 Hz, 1H), 1.96 (dt, J = 24.4, 11.0 Hz, 8H), |
| | 1.72 (s, 4H), 1.51 (t, J = 8.8 Hz, 2H), 0.85 (t, J = 6.6 Hz, 1H), 0.78 (q, J = |
| | 5.2 Hz, 1H), 0.69 (dt, J = 9.2, 4.7 Hz, 1H), 0.63 (d, J = 6.0 Hz, 1H), |
| | 0.41 (s, 1H). |
| 351 | ¹H NMR (400 MHz, DMSO-d₆) δ 13.05 (s, 1H), 8.35 (s, 1H), 7.88 (s, |
| | 1H), 7.66 (s, 2H), 7.25 (s, 1H), 7.12 (s, 2H), 6.38 (s, 1H), 5.06 (dd, J = |
| | 10.5, 4.4 Hz, 1H), 4.39 (s, 1H), 4.20-4.03 (m, 2H), 3.27-3.16 (m, 1H), |
| | 2.17 (d, J = 6.0 Hz, 2H), 2.11-2.03 (m, 3H), 1.54 (dd, J = 16.5, 9.4 Hz, |
| | 1H), 0.80 (s, 1H), 0.76 (s, 1H), 0.66 (s, 1H), 0.53-0.44 (m, 4H). |
| 352 | ¹H NMR (400 MHz, DMSO-d₆) δ 13.04 (s, 1H), 8.45 (s, 1H), 7.93 (s, |
| | 1H), 7.68 (s, 2H), 7.25 (t, J = 7.8 Hz, 1H), 7.17-7.01 (m, 2H), 6.40 (s, |
| | 1H), 5.10 (dd, J = 10.6, 4.4 Hz, 1H), 4.31 (t, J = 11.3 Hz, 1H), 4.07 (p, J = |
| | 8.7 Hz, 1H), 3.77-3.64 (m, 1H), 3.30-3.22 (m, 2H), 3.22-3.18 (m, |
| | 2H), 3.17-3.13 (m, 2H), 2.48-2.38 (m, 2H), 2.21-1.80 (m, 6H), 1.62 |
| | (dd, J = 15.1, 8.4 Hz, 1H), 1.39 (d, J = 14.9 Hz, 1H), 0.50 (s, 9H). |
| 353 | ¹H NMR (400 MHz, DMSO-d₆) δ 13.0 (s, 1H), 8.40 (s, 1H), 8.00-7.84 |
| | (m, 2H), 7.66 (s, 2H), 7.25 (s, 1H), 7.11 (s, 2H), 6.39 (s, 1H), 5.14-4.97 |
| | (m, 1H), 4.38-4.18 (m, 1H), 4.09 (p, J = 8.2 Hz, 1H), 3.96-3.83 (m, |
| | 1H), 3.71-3.63 (m, 1H), 3.08 (t, J = 9.6 Hz, 1H), 2.99 (t, J = 9.9 Hz, |
| | 1H), 2.46-2.39 (m, 1H), 2.37-2.31 (m, 1H), 2.30-2.24 (m, 1H), 2.12 |
| | (d, J = 9.9 Hz, 3H), 2.03 (q, J = 7.5 Hz, 2H), 2.00-1.82 (m, 6H), 1.65- |
| | 1.55 (m, 1H), 1.37 (d, J = 14.8 Hz, 1H), 0.97 (t, J = 7.6 Hz, 3H), 0.49 (s, |
| | 9H). |
| 354 | ¹H NMR (400 MHz, DMSO-d₆) δ 13.03 (s, 1H), 8.41 (s, 1H), 7.91 (s, |
| | 1H), 7.66 (s, 2H), 7.24 (t, J = 7.8 Hz, 1H), 7.12 (s, 2H), 6.39 (s, 1H), 5.06 |
| | (dd, J = 10.7, 4.3 Hz, 1H), 4.42 (td, J = 5.4, 3.4 Hz, 2H), 4.28 (t, J = 11.0 |
| | Hz, 1H), 3.94-3.80 (m, 1H), 3.72-3.57 (m, 2H), 3.00 (t, J = 9.6 Hz, |
| | 1H), 2.94 (t, J = 9.7 Hz, 1H), 2.56-2.52 (m, 2H), 2.29-2.19 (m, 2H), |
| | 2.19-2.00 (m, 3H), 2.00-1.83 (m, 6H), 1.80 (s, 2H), 1.58 (dd, J = 15.1, |
| | 8.3 Hz, 1H), 1.36 (d, J = 14.9 Hz, 1H), 0.49 (s, 9H). |
| 355 | ¹H NMR (400 MHz, DMSO-d₆) δ 8.39 (s, 1H), 7.81-7.72 (m, 1H), 7.52- |
| | 7.38 (m, 2H), 7.34 (d, J = 7.9 Hz, 1H), 7.15-7.07 (m, 1H), 7.05-6.96 |
| | (m, 2H), 5.73 (s, 1H), 5.04-4.95 (m, 1H), 4.82-4.73 (m, 1H), 3.92-3.76 |
| | (m, 3H), 3.10-3.07 (m, 2H), 2.98 (t, J = 10.3 Hz, 2H), 2.24-2.17 (m, |
| | 4H), 2.11-2.08 (m, 1H), 1.97-1.92 (m, 6H), 1.52-1.48 (m, 4H), 1.39- |
| | 1.35 (m, 2H), 0.89-0.83 (m, 2H), 0.46 (s, 9H). (sulfonamide N—H not |
| | seen) |
| 356 | ¹H NMR (400 MHz, DMSO-d₆) δ 8.40 (s, 1H), 7.89 (s, 1H), 7.64 (s, 2H), |
| | 7.37 (d, J = 7.9 Hz, 1H), 7.28-7.00 (m, 3H), 6.42 (s, 1H), 5.13-4.96 (m, |
| | 1H), 4.36-3.99 (m, 1H), 3.92-3.84 (m, 2H), 3.78-3.59 (m, 1H), 3.05 |
| | (t, J = 9.6 Hz, 1H), 2.98 (t, J = 9.9 Hz, 1H), 2.44-2.37 (m, 1H), 2.28- |
| | 2.21 (m, 1H), 2.14-2.07 (m, 2H), 2.03-1.87 (m, 6H), 1.62-1.53 (m, |
| | 1H), 1.39-1.33 (m, 1H), 1.27-1.21 (m, 1H), 1.15 (d, J = 6.6 Hz, 2H), |
| | 0.66-0.52 (m, 4H), 0.48 (s, 9H). (sulfonamide N—H is missing) |
| 357 | ¹H NMR (400 MHz, DMSO-d₆) δ 8.40 (s, 1H), 7.88 (s, 1H), 7.63 (s, 2H), |
| | 7.32 (d, J = 7.9 Hz, 1H), 7.09 (s, 3H), 6.42 (s, 1H), 5.10-4.93 (m, 1H), |
| | 3.95 (q, J = 7.1 Hz, 2H), 3.91-3.82 (m, 2H), 3.71-3.62 (m, 2H), 3.07 |
| | (t, J = 9.5 Hz, 1H), 2.97 (t, J = 9.8 Hz, 1H), 2.42-2.38 (m, 1H), 2.28- |
| | 2.21 (m, 1H), 2.14-2.06 (m, 2H), 2.03-1.83 (m, 7H), 1.62-1.46 (m, |
| | 2H), 1.39-1.33 (m, 1H), 1.15 (t, J = 6.8 Hz, 3H), 0.48 (s, 9H). |
| | (integration did not pick sulfonamide N—H around 13 ppm) |
| 358 | ¹H NMR (400 MHz, DMSO-d₆) δ 12.99 (s, 1H), 9.74 (s, 1H), 8.47 (s, |
| | 1H), 7.93 (d, J = 7.0 Hz, 1H), 7.75-7.60 (m, 2H), 7.29-7.21 (m, 1H), |
| | 7.12 (d, J = 7.7 Hz, 2H), 6.39 (s, 1H), 5.19 (dd, J = 10.7, 4.2 Hz, 1H), |
| | 4.72 (t, J = 5.7 Hz, 1H), 4.29 (t, J = 11.2 Hz, 1H), 3.86 (q, J = 8.4 Hz, |
| | 1H), 3.77-3.64 (m, 1H), 3.50 (d, J = 4.9 Hz, 2H), 3.43-3.39 (m, 1H), |
| | 3.08-2.95 (m, 2H), 2.68-2.54 (m, 2H), 2.01 (s, 6H), 1.87 (s, 6H), 1.70- |
| | 1.55 (m, 1H), 1.33-1.22 (m, 1H), 1.22-1.13 (m, 1H), 0.75 (d, J = 6.5 |
| | Hz, 3H), 0.24 (d, J = 6.2 Hz, 3H). |
| 61 | ¹H NMR (400 MHz, DMSO-d₆) δ 13.0 (s, 1H), 8.41 (s, 1H), 7.95-7.84 |
| | (m, 1H), 7.76-7.59 (m, 2H), 7.24 (d, J = 7.7 Hz, 1H), 7.12 (d, J = 7.6 |
| | Hz, 2H), 6.36 (s, 1H), 5.08 (d, J = 9.2 Hz, 1H), 4.32-4.13 (m, 2H), 4.12- |
| | 4.01 (m, 1H), 3.30 (s, 3H), 3.03 (s, 3H), 2.21-2.06 (m, 4H), 2.05-1.77 |
| | (m, 6H), 1.48 (dd, J = 16.3, 8.7 Hz, 2H), 0.87-0.80 (m, 2H), 0.78-0.72 |
| | (m, 1H), 0.67-0.57 (m, 1H), 0.57-0.45 (m, 1H). |
| 360 | ¹H NMR (400 MHz, DMSO-d₆) δ 13.03 (s, 1H), 9.74 (s, 1H), 8.43 (s, |
| | 1H), 7.91 (d, J = 7.2 Hz, 1H), 7.68 (s, 2H), 7.51-7.43 (m, 2H), 7.32- |
| | 7.21 (m, 3H), 7.12 (d, J = 7.6 Hz, 2H), 6.98 (tt, J = 7.3, 1.1 Hz, 1H), 6.38 |
| | (s, 1H), 5.16 (dd, J = 10.7, 4.2 Hz, 1H), 4.76 (p, J = 7.4 Hz, 1H), 4.32 (t, |
| | J = 11.1 Hz, 1H), 3.83-3.63 (m, 2H), 3.29-3.19 (m, 2H), 2.65-2.52 |
| | (m, 2H), 2.22-1.80 (m, 6H), 1.67 (t, J = 12.5 Hz, 1H), 1.35-1.23 (m, |
| | 1H), 1.21-1.10 (m, 1H), 0.75 (d, J = 6.6 Hz, 3H), 0.22 (d, J = 6.3 Hz, |
| | 3H). |
| 361 | ¹H NMR (400 MHz, DMSO-d₆) δ 13.06 (s, 1H), 8.41 (s, 1H), 7.90 (s, |
| | 1H), 7.67 (s, 2H), 7.25 (d, J = 7.9 Hz, 1H), 7.18-7.01 (m, 3H), 6.38 (s, |
| | 1H), 5.23-5.05 (m, 1H), 4.62 (p, J = 7.3 Hz, 1H), 4.30 (t, J = 11.1 Hz, |
| | 1H), 3.80-3.59 (m, 2H), 3.17-3.03 (m, 2H), 2.60-2.51 (m, 5H) (N—Me |
| | and CH2), 2.21-1.85 (m, 6H), 1.65 (t, J = 12.5 Hz, 1H), 1.28 (s, 1H), |
| | 1.14 (t, J = 11.8 Hz, 1H), 0.74 (d, J = 6.6 Hz, 3H), 0.21 (d, J = 6.5 Hz, |
| | 3H). |
| 59 | ¹H NMR (400 MHz, DMSO-d₆) δ 13.02 (s, 1H), 8.36 (s, 1H), 7.89 (s, |
| | 1H), 7.78-7.56 (m, 2H), 7.42 (d, J = 7.1 Hz, 1H), 7.25 (d, J = 8.7 Hz, |
| | 1H), 7.12 (s, 2H), 6.38 (s, 1H), 5.07 (d, J = 10.4 Hz, 1H), 4.76 (hept, J = |
| | 6.2 Hz, 1H), 4.25-4.05 (m, 4H), 3.78-3.65 (m, 2H), 3.66-3.58 (m, 1H), |
| | 3.23-3.05 (m, 2H), 2.21-2.05 (m, 5H), 2.02-1.87 (m, 3H), 1.46 (dd, J = |
| | 16.5, 9.1 Hz, 1H), 1.18 (d, J = 6.2 Hz, 4H), 0.87-0.70 (m, 2H), 0.69- |
| | 0.57 (m, 1H), 0.56-0.43 (m, 1H). |
| 362 | ¹H NMR (400 MHz, DMSO-d₆) δ 12.83 (s, 1H), 8.21 (s, 1H), 7.70 (s, |
| | 1H), 7.59 (t, J = 6.2 Hz, 1H), 7.54-7.38 (m, 2H), 7.14-7.09 (m, 2H), |
| | 7.07-7.01 (m, 4H), 6.91 (d, J = 7.6 Hz, 2H), 6.17 (s, 1H), 5.07-4.82 (m, |
| | 1H), 4.44 (p, J = 7.4 Hz, 1H), 4.10 (t, J = 11.1 Hz, 1H), 3.97 (d, J = 6.3 |
| | Hz, 2H), 3.56-3.40 (m, 2H), 2.93 (q (likely two doublets), J = 9.3 Hz, |
| | 2H), 2.39-2.30 (m, 2H), 1.75 (s, 6H), 1.45 (t, J = 12.5 Hz, 1H), 1.13- |
| | 1.00 (m, 1H), 0.98-0.87 (m, 1H), 0.53 (d, J = 6.6 Hz, 3H), 0.01 (d, J = |
| | 6.4 Hz, 3H). |
| 363 | ¹H NMR (400 MHz, DMSO-d₆) δ 12.82 (s, 1H), 8.19 (s, 1H), 7.68 (s, |
| | 1H), 7.45 (s, 2H), 7.08-6.94 (m, 2H), 6.90 (d, J = 7.6 Hz, 2H), 6.16 (s, |
| | 1H), 4.92 (dd, J = 10.8, 4.2 Hz, 1H), 4.39 (p, J = 7.4 Hz, 1H), 4.08 (t, J = |
| | 11.1 Hz, 1H), 3.56-3.36 (m, 2H), 2.89 (q (probably two doublets), J = |
| | 9.1 Hz, 2H), 2.59 (t, J = 6.2 Hz, 2H), 2.38-2.28 (m, 2H), 2.00-1.57 |
| | (m, 6H), 1.48-1.35 (m, 6H), 1.22-1.02 (m, 2H), 1.00-0.84 (m, 4H), |
| | 0.73-0.56 (m, 2H), 0.52 (d, J = 6.6 Hz, 3H), −0.01 (d, J = 6.4 Hz, 3H). |
| 364 | ¹H NMR (400 MHz, DMSO-d₆) δ 13.05 (s, 1H), 8.42 (s, 1H), 7.91 (s, |
| | 1H), 7.68 (s, 2H), 7.28-7.21 (m, 1H), 7.20-7.04 (m, 3H), 6.38 (s, 1H), |
| | 5.22-5.02 (m, 2H), 4.36-4.20 (m, 2H), 3.77-3.65 (m, 1H), 3.24 (s, 3H), |
| | 3.23-3.19 (m, 1H), 3.16-3.08 (m, 4H), 2.46-2.22 (m, 2H), 2.15-1.83 |
| | (m, 6H), 1.70-1.50 (m, 1H), 1.33-1.22 (m, 1H), 1.19-1.12 (m, 1H), |
| | 0.84 (dd, J = 10.5, 6.6 Hz, 1H), 0.73 (two d, J = 13.4, 6.6 Hz, 3H), 0.20 |
| | (two d, J = 8.3 Hz, 3H). |
| 365 | ¹H NMR (400 MHz, DMSO-d₆) δ 13.05 (s, 1H), 8.41 (s, 1H), 7.90 (s, |
| | 1H), 7.66 (s, 2H), 7.24 (t, J = 7.7 Hz, 1H), 7.18 (t, J = 5.6 Hz, 1H), 7.12 |
| | (d, J = 7.6 Hz, 2H), 6.37 (s, 1H), 5.14 (dd, J = 10.7, 4.2 Hz, 1H), 4.62 (p, |
| | J = 7.4 Hz, 1H), 4.30 (t, J = 11.2 Hz, 1H), 3.81-3.60 (m, 2H), 3.11 (q, J = |
| | 9.1 Hz, 2H), 3.03-2.89 (m, 2H), 2.15-1.84 (m, 6H), 1.65 (t, J = 12.6 |
| | Hz, 1H), 1.36-1.23 (m, 1H), 1.19-1.08 (m, 1H), 1.01 (t, J = 7.2 Hz, |
| | 3H), 0.74 (d, J = 6.6 Hz, 3H), 0.22 (d, J = 6.3 Hz, 3H). |
| 366 | ¹H NMR (400 MHz, DMSO-d₆) δ 13.0 (s, 1H), 8.41 (s, 1H), 8.23 (d, J = |
| | 6.7 Hz, 1H), 7.97-7.86 (m, 1H), 7.78-7.58 (m, 2H), 7.31-7.20 (m, 1H), |
| | 7.12 (d, J = 7.4 Hz, 2H), 6.38 (s, 1H), 5.19-5.07 (m, 1H), 4.27-4.17 (m, |
| | 2H), 3.79-3.66 (m, 1H), 3.19-3.10 (m, 2H), 2.18-2.08 (m, 3H), 2.07- |
| | 1.87 (m, 6H), 1.83 (s, 3H), 1.55 (t, J = 12.4 Hz, 1H), 1.31-1.23 (m, 1H), |
| | 1.16 (t, J = 12.3 Hz, 1H), 0.73 (d, J = 6.6 Hz, 3H), 0.20 (d, J = 6.2 Hz, |
| | 3H). |
| 367 | ¹H NMR (400 MHz, DMSO-d₆) δ 13.07 (s, 1H), 9.27-9.07 (m, 2H), 8.44 |
| | (d, J = 11.3 Hz, 1H), 7.92 (s, 1H), 7.69 (s, 2H), 7.25 (d, J = 7.8 Hz, 1H), |
| | 7.12 (d, J = 7.3 Hz, 2H), 6.39 (s, 1H), 5.15 (dd, J = 10.8, 4.3 Hz, 1H), |
| | 4.58 (p, J = 8.5 Hz, 1H), 4.22 (t, J = 11.2 Hz, 1H), 4.01 (s, 1H), 3.81- |
| | 3.69 (m, 1H), 3.31-3.26 (m, 1H), 3.21 (td, J = 12.2, 6.3 Hz, 2H), 2.57- |
| | 2.51 (m, 1H), 2.48-2.41 (m, 1H), 2.12-1.85 (m, 6H), 1.56 (t, J = 12.4 |
| | Hz, 1H), 1.25 (two superimposed d, J = 6.5, Hz, 6H), 1.24-1.12 (m, 2H), |
| | 0.72 (d, J = 6.5 Hz, 3H), 0.20 (d, J = 6.2 Hz, 3H). |
| 389 | ¹H NMR (500 MHz, DMSO-d₆) δ 8.19 (s, 1H), 7.84 (d, J = 7.8 Hz, 1H), |
| | 7.64 (t, J = 7.7 Hz, 1H), 7.57 (d, J = 7.5 Hz, 1H), 7.23 (t, J = 7.7 Hz, 1H), |
| | 7.09 (d, J = 7.6 Hz, 2H), 6.82 (s, 1H), 6.24 (s, 1H), 4.93 (dd, J = 12.0, |
| | 3.4 Hz, 1H), 4.45 (t, J = 11.8 Hz, 1H), 4.17 (p, J = 8.5 Hz, 1H), 3.42 (s, |
| | 1H), 3.31-3.24 (m, 2H), 2.16-2.09 (m, 2H), 1.96 (s, 6H), 1.66-1.56 |
| | (m, 1H), 1.37-1.26 (m, 1H), 1.24-1.14 (m, 1H), 0.73 (d, J = 6.6 Hz, |
| | 3H), 0.55-0.50 (m, 2H), 0.47 (ddt, J = 11.1, 7.7, 4.1 Hz, 2H), 0.13 (d, J = |
| | 6.3 Hz, 3H). |
| 401 | ¹H NMR (500 MHz, DMSO-d₆) δ 8.21 (t, J = 7.8 Hz, 1H), 8.09 (d, J = |
| | 8.0 Hz, 1H), 7.79 (d, J = 7.5 Hz, 1H), 7.26 (t, J = 7.6 Hz, 1H), 7.13 (d, J = |
| | 7.7 Hz, 2H), 6.33 (s, 1H), 5.70-5.62 (m, 1H), 4.30 (t, J = 8.4 Hz, 1H), |
| | 4.12 (t, J = 10.7 Hz, 1H), 3.69-3.57 (m, 1H), 3.35 (dt, J = 18.6, 9.2 Hz, |
| | 2H), 2.17 (dd, J = 15.1, 8.2 Hz, 2H), 2.03 (s, 6H), 1.65 (dd, J = 15.1, 8.5 |
| | Hz, 1H), 1.48 (d, J = 14.9 Hz, 1H), 0.54 (s, 9H), 0.53-0.44 (m, 4H). |
| 402 | ¹H NMR (400 MHz, DMSO-d₆) δ 12.72 (broad s, 1H), 8.41 (s, 1H), 7.90 |
| | (s, 1H), 7.65 (d, J = 5.5 Hz, 2H), 7.34 (d, J = 4.4 Hz, 2H), 7.26 (d, J = |
| | 8.0 Hz, 1H), 7.21-7.10 (m, 1H), 6.35 (s, 1H), 5.05 (d, J = 10.8, 4.4 Hz, |
| | 1H), 4.72 (p, J = 6.3 Hz, 1H), 4.41-4.01 (m, 3H), 3.98-3.78 (m, 2H), |
| | 3.67 (s, 1H), 3.06 (t, J = 9.7 Hz, 1H), 2.96 (t, J = 9.9 Hz, 1H), 2.44-2.19 |
| | (m, 3H), 2.18-1.83 (m, 6H), 1.59 (dd, J = 15.0, 8.3 Hz, 1H), 1.36 (d, J = |
| | 15.0 Hz, 1H), 1.21-1.06 (m, 6H), 0.50 (s, 9H). |
| 403 | ¹H NMR (400 MHz, DMSO-d₆) δ 13.53-11.61 (Broad m, 1H), 8.42 (s, |
| | 1H), 7.91 (s, 1H), 7.65 (s, 2H), 7.34 (d, J = 4.0 Hz, 2H), 7.26 (d, J = 7.8 |
| | Hz, 1H), 7.18 (s, 1H), 6.37 (s, 1H), 5.06 (dd, J = 11.3, 4.4 Hz, 1H), 4.72 |
| | (p, J = 6.3 Hz, 1H), 4.46-3.99 (m, 3H), 3.99-3.78 (m, 2H), 3.67 (br s, |
| | 1H), 3.09-2.87 (m, 2H), 2.45-2.36 (m, 1H), 2.34-1.80 (m, 8H), 1.57 |
| | (dd, J = 15.0, 8.3 Hz, 1H), 1.36 (d, J = 14.9 Hz, 1H), 1.15 (d, J = 6.2 Hz, |
| | 6H), 0.49 (s, 9H). |
| 4 | ¹H NMR (400 MHz, DMSO-d₆) δ 13.19-11.69 (broad m, 1H), 8.36 (s, |
| | 1H), 7.88 (s, 1H), 7.64 (s, 2H), 7.24 (d, J = 7.8 Hz, 1H), 7.12 (d, J = 7.5 |
| | Hz, 2H), 6.36 (s, 1H), 5.10 (dd, J = 11.0, 3.9 Hz, 1H), 4.34 (t, J = 11.2 |
| | Hz, 1H), 3.98 (s, 1H), 3.82 (p, J = 8.6 Hz, 1H), 3.69-3.55 (m, 1H), 2.90 |
| | (t, J = 9.6 Hz, 1H), 2.82 (t, J = 9.8 Hz, 1H), 2.32-2.25 (m, 1H), 2.19- |
| | 1.88 (m, 11H), 1.80 (t, J = 9.5 Hz, 1H), 1.68-1.53 (m, 1H), 1.53-1.37 |
| | (m, 1H), 1.18-0.88 (m, 8H), 0.83-0.71 (m, 1H), 0.68 (d, J = 6.4 Hz, |
| | 3H), 0.60 (d, J = 6.4 Hz, 3H). |
| 5 | ¹H NMR (400 MHz, DMSO-d₆) δ 13.45-11.43 (broad m, 1H), 8.36 (s, |
| | 1H), 7.88 (s, 1H), 7.65 (s, 2H), 7.32-7.19 (m, 1H), 7.12 (d, J = 7.7 Hz, |
| | 2H), 6.36 (s, 1H), 5.09 (dd, J = 10.8, 3.8 Hz, 1H), 4.32 (t, J = 11.2 Hz, |
| | 1H), 3.99 (s, 1H), 3.82 (p, J = 8.6 Hz, 1H), 3.59 (d, J = 12.0 Hz, 1H), |
| | 2.92 (t, J = 9.5 Hz, 1H), 2.80 (t, J = 9.8 Hz, 1H), 2.32-2.26 (m, 1H), |
| | 2.20-1.88 (m, 11H), 1.85-1.74 (m, 1H), 1.70-1.53 (m, 1H), 1.53-1.39 |
| | (m, 1H), 1.14-0.90 (m, 8H), 0.86-0.73 (m, 1H), 0.68 (d, J = 6.3 Hz, |
| | 3H), 0.61 (d, J = 6.3 Hz, 3H). |
| 408 | ¹H NMR (400 MHz, DMSO-d₆) δ 13.47-11.67 (broad m, 1H), 8.41 (s, |
| | 1H), 7.91 (s, 1H), 7.65 (s, 2H), 7.32-7.21 (m, 1H), 7.12 (s, 2H), 6.39 (s, |
| | 1H), 5.07 (dd, J = 10.9, 4.3 Hz, 1H), 4.31 (t, J = 11.1 Hz, 1H), 3.86 (p, J = |
| | 8.6 Hz, 1H), 3.71 (s, 1H), 3.70-3.61 (m, 1H), 3.02-2.82 (m, 2H), 2.35- |
| | 1.79 (m, 12H), 1.79-1.66 (m, 1H), 1.59 (dd, J = 15.0, 8.3 Hz, 1H), 1.43- |
| | 1.09 (m, 5H), 0.80-0.68 (m, 6H), 0.49 (s, 9H). |
| 409 | ¹H NMR (400 MHz, DMSO-d₆) δ 13.47-11.55 (broad m, 1H), 8.40 (s, |
| | 1H), 7.91 (s, 1H), 7.66 (s, 2H), 7.32-7.20 (m, 1H), 7.20-7.01 (m, 2H), |
| | 6.39 (s, 1H), 5.06 (dd, J = 10.8, 4.3 Hz, 1H), 4.28 (t, J = 11.1 Hz, 1H), |
| | 3.87 (p, J = 8.7 Hz, 1H), 3.72 (s, 1H), 3.70-3.61 (m, 1H), 3.02 (t, J = |
| | 9.5 Hz, 1H), 2.85 (t, J = 9.9 Hz, 1H), 2.42-2.29 (m, 1H), 2.29-1.81 (m, |
| | 11H), 1.79-1.69 (m, 1H), 1.62 (dd, J = 15.2, 8.3 Hz, 1H), 1.42-1.11 (m, |
| | 5H), 0.82-0.67 (m, 6H), 0.49 (s, 9H). |
| 410 | ¹H NMR (400 MHz, DMSO-d₆) δ 13.38-11.49 (broaad m, 1H), 8.19 (s, |
| | 1H), 8.07 (s, 1H), 7.79 (s, 1H), 7.27 (t, J = 7.7 Hz, 1H), 7.13 (s, 2H), 6.40 |
| | (s, 1H), 5.68-5.51 (m, 1H), 4.27 (br s, 1H), 4.04-3.85 (m, 2H), 3.62- |
| | 3.49 (m, 1H), 3.05-2.82 (m, 2H), 2.41-1.71 (m, 9H), 1.50 (dd, J = 15.0, |
| | 7.9 Hz, 1H), 1.38 (d, J = 14.9 Hz, 1H), 1.10 (s, 3H), 1.09 (s, 3H), 0.50 (s, |
| | 9H). |
| 411 | ¹H NMR (400 MHz, DMSO-d₆) δ 13.48-11.78 (broad m, 1H), 8.18 (s, |
| | 1H), 8.13-7.97 (m, 1H), 7.76 (s, 1H), 7.27 (t, J = 7.6 Hz, 1H), 7.13 (s, |
| | 2H), 6.39 (s, 1H), 5.74-5.52 (m, 1H), 4.41-3.85 (m, 3H), 3.61-3.48 |
| | (m, 1H), 2.90-2.64 (m, 2H), 2.31-1.74 (m, 9H), 1.68 (dd, J = 15.1, 8.2 |
| | Hz, 1H), 1.42 (d, J = 15.2 Hz, 1H), 1.06 (s, 6H), 0.51 (s, 9H). |
| 412 | ¹H NMR (400 MHz, DMSO-d₆) δ 13.41-11.66 (broad m, 1H), 8.37 (s, |
| | 1H), 7.89 (s, 1H), 7.65 (s, 2H), 7.33-7.19 (m, 1H), 7.12 (d, J = 7.6 Hz, |
| | 2H), 6.36 (s, 1H), 5.11 (dd, J = 10.9, 4.2 Hz, 1H), 4.34 (t, J = 11.1 Hz, |
| | 1H), 3.98 (s, 1H), 3.83 (p, J = 8.6 Hz, 1H), 3.68 (t, J = 10.6 Hz, 1H), 2.94 |
| | (t, J = 9.6 Hz, 1H), 2.83 (t, J = 9.8 Hz, 1H), 2.38-2.24 (m, 1H), 2.21- |
| | 1.84 (m, 11H), 1.80 (t, J = 9.5 Hz, 1H), 1.62 (t, J = 12.4 Hz, 1H), 1.28 (s, |
| | 1H), 1.14 (t, J = 12.2 Hz, 1H), 0.97 (s, 3H), 0.96 (s, 3H), 0.73 (d, J = 6.6 |
| | Hz, 3H), 0.20 (d, J = 6.3 Hz, 3H). |
| 413 | ¹H NMR (400 MHz, DMSO-d₆) δ 13.31-11.63 (broad m, 1H), 8.37 (s, |
| | 1H), 7.89 (s, 1H), 7.66 (br s, 2H), 7.25 (t, J = 7.5 Hz, 1H), 7.11 (d, J = |
| | 7.6 Hz, 2H), 6.36 (s, 1H), 5.10 (dd, J = 10.9, 4.1 Hz, 1H), 4.33 (t, J = |
| | 11.1 Hz, 1H), 3.99 (s, 1H), 3.83 (p, J = 8.6 Hz, 1H), 3.68 (t, J = 12.9 Hz, |
| | 1H), 2.94 (t, J = 9.5 Hz, 1H), 2.84 (t, J = 9.8 Hz, 1H), 2.31-2.22 (m, |
| | 1H), 2.17-1.87 (m, 11H), 1.84-1.74 (m, 1H), 1.64 (t, J = 12.3 Hz, 1H), |
| | 1.29 (br s, 1H), 1.14 (t, J = 12.1 Hz, 1H), 0.96 (s, 6H), 0.74 (d, J = 6.6 |
| | Hz, 3H), 0.21 (d, J = 6.2 Hz, 3H). |
| 414 | ¹H NMR (400 MHz, DMSO-d₆) δ 13.41-11.77 (broad m, 1H), 8.18 (s, |
| | 1H), 8.06 (s, 1H), 7.76 (s, 1H), 7.27 (t, J = 7.5 Hz, 1H), 7.13 (s, 2H), 6.39 |
| | (s, 1H), 5.65-5.49 (m, 1H), 4.10 (t, J = 10.6 Hz, 1H), 3.94 (p, J = 8.8 |
| | Hz, 1H), 3.58-3.47 (m, 1H), 3.22 (p, J = 8.6 Hz, 1H), 3.13 (t, J = 9.6 |
| | Hz, 1H), 3.01 (t, J = 9.8 Hz, 1H), 2.88 (s, 3H), 2.80 (s, 3H), 2.44-2.37 |
| | (m, 1H), 2.35-1.79 (m, 11H), 1.58 (dd, J = 15.2, 8.2 Hz, 1H), 1.39 (d, J = |
| | 14.9 Hz, 1H), 0.50 (s, 9H). |
| 415 | ¹H NMR (400 MHz, DMSO-d₆) δ 13.36-11.88 (broad m, 1H), 8.18 (s, |
| | 1H), 8.06 (s, 1H), 7.76 (s, 1H), 7.27 (t, J = 7.5 Hz, 1H), 7.13 (s, 2H), 6.39 |
| | (s, 1H), 5.66-5.49 (m, 1H), 4.12 (t, J = 10.5 Hz, 1H), 3.94 (p, J = 8.7 |
| | Hz, 1H), 3.58-3.46 (m, 1H), 3.24 (p, J = 8.6 Hz, 1H), 3.13 (t, J = 9.6 |
| | Hz, 1H), 3.02 (t, J = 9.8 Hz, 1H), 2.88 (s, 3H), 2.80 (s, 3H), 2.40-1.79 |
| | (m, 12H), 1.55 (dd, J = 15.1, 8.2 Hz, 1H), 1.39 (d, J = 14.9 Hz, 1H), 0.49 |
| | (s, 9H). |
| 418 | ¹H NMR (400 MHz, DMSO-d₆) δ 12.65-12.24 (broad m, 1H), 8.45 (s, |
| | 1H), 7.93 (s, 1H), 7.70 (br s, 2H), 7.54 (d, J = 6.9 Hz, 1H), 7.25 (t, J = |
| | 7.8 Hz, 1H), 7.12 (d, J = 7.6 Hz, 2H), 6.41 (br s, 1H), 5.20-5.06 (m, 1H), |
| | 4.26 (p, J = 11.4, 10.0 Hz, 2H), 4.18-4.06 (m, 1H), 3.83 (br s, 1H), 3.55 |
| | (s, 3H), 3.26-3.09 (m, 2H), 2.28-1.65 (m, 10H), 0.83 (s, 3H), 0.60 (s, |
| | 3H). |
| 419 | ¹H NMR (400 MHz, DMSO-d₆) δ 13.48-11.43 (broad m, 1H), 8.45 (s, |
| | 1H), 7.93 (s, 1H), 7.69 (s, 2H), 7.46 (d, J = 7.3 Hz, 1H), 7.34-7.22 (m, |
| | 1H), 7.22-7.01 (m, 2H), 6.46 (s, 1H), 5.16 (d, J = 9.7 Hz, 1H), 4.34 (t, J = |
| | 11.2 Hz, 1H), 3.92-3.66 (m, 3H), 3.52 (s, 3H), 2.84 (dq, J = 18.5, 9.3 |
| | Hz, 2H), 2.50 (m, 2H overlapped with DMSO), 2.27-1.80 (m, 7H), 1.75 |
| | (d, J = 15.8 Hz, 1H), 0.87 (s, 3H), 0.64 (s, 3H). |
| 424 | ¹H NMR (400 MHz, DMSO-d₆) δ 13.49-13.00 (very broad doublet, 1H) |
| | 8.41 (s, 1H), 7.92 (br s, 1H), 7.71 (br s, 2H), 7.16 (br d, J = 7.6 Hz, 2H), |
| | 6.45 (br s, 1H), 5.12 (dd, J = 10.7, 4.2 Hz, 1H), 4.39 (t, J = 11.1 Hz, 1H), |
| | 4.23 (p, J = 8.4 Hz, 1H), 3.71 (t, J = 11.2 Hz, 1H), 3.27 (td, J = 9.5, 8.6, |
| | 3.4 Hz, 2H), 2.25-1.74 (m, 8H), 1.67 (t, J = 12.6 Hz, 1H), 1.30 (br s, |
| | 1H), 1.15 (dd, J = 13.6, 10.3 Hz, 1H), 0.72 (d, J = 6.6 Hz, 3H), 0.56 - |
| | 0.39 (m, 4H), 0.20 (d, J = 6.3 Hz, 3H). |
| 429 | ¹H NMR (400 MHz, DMSO-d₆) δ 13.09 (s, 1H), 8.69 (s, 1H), 7.94 (s, |
| | 2H), 7.72 (s, 2H), 7.57 (s, 1H), 7.25 (d, J = 7.7 Hz, 1H), 7.12 (d, J = 7.5 |
| | Hz, 2H), 6.37 (s, 1H), 5.52-5.39 (m, 1H), 5.00 (s, 2H), 3.98 (d, J = 11.5 |
| | Hz, 1H), 3.88 (t, J = 11.1 Hz, 1H), 1.99 (t, J = 32.8 Hz, 6H), 1.44 (s, 9H), |
| | 1.38-1.31 (m, 1H), 1.27 (d, J = 24.5 Hz, 1H), 1.03 (t, J = 12.3 Hz, 1H), |
| | 0.74 (d, J = 6.6 Hz, 3H), 0.23 (d, J = 6.3 Hz, 3H). |
| 442 | ¹H NMR (500 MHz, DMSO-d₆) δ 12.92 (s, 1H), 8.34 (s, 1H), 7.84 (s, |
| | 1H), 7.59 (d, J = 28.2 Hz, 2H), 7.25 (s, 1H), 7.12 (d, J = 7.6 Hz, 2H), |
| | 6.21 (s, 1H), 3.132.98 (m, 1H), 2.61 (d, J = 24.3 Hz, 2H), 2.462.16 |
| | (m, 1H), 2.04 (d, J = 30.8 Hz, 7H), 1.871.79 (m, 2H), 1.72 (dd, J = |
| | 32.8, 12.0 Hz, 2H), 1.54 (s, 3H), 0.92 (t, J = 11.8 Hz, 2H), 0.28 (dd, J = |
| | 18.5, 6.5 Hz, 4H). |
| 77 | ¹H NMR (400 MHz, DMSO-d₆) δ 13.03 (s, 1H), 8.33 (d, J = 6.0 Hz, 1H), |
| | 7.88 (d, J = 7.5 Hz, 1H), 7.74-7.55 (m, 2H), 7.26 (t, J = 7.7 Hz, 1H), |
| | 7.09 (dd, J = 23.0, 7.8 Hz, 3H), 6.37 (s, 1H), 5.04 (dt, J = 10.5, 4.9 Hz, |
| | 1H), 4.33 (td, J = 11.5, 5.5 Hz, 1H), 4.06 (s, 1H), 3.79 (tt, J = 17.3, 8.4 |
| | Hz, 2H), 3.05-2.86 (m, 2H), 2.37 (dt, J = 12.3, 6.2 Hz, 1H), 2.29-2.02 |
| | (m, 7H), 1.93 (q, J = 8.1, 6.7 Hz, 5H), 1.48 (ddd, J = 15.7, 9.3, 6.0 Hz, |
| | 1H), 1.37 (s, 9H), 0.78 (ddq, J = 19.2, 9.5, 4.7, 4.3 Hz, 2H), 0.70-0.50 |
| | (m, 2H). |
| 89 | ¹H NMR (400 MHz, DMSO-d₆) δ 13.03 (s, 1H), 8.40 (s, 1H), 7.91 (s, |
| | 1H), 7.65 (s, 2H), 7.25 (t, J = 7.7 Hz, 1H), 7.08 (dd, J = 21.2, 7.8 Hz, |
| | 3H), 6.38 (s, 1H), 5.05 (dd, J = 10.7, 4.3 Hz, 1H), 4.27 (t, J = 11.3 Hz, |
| | 1H), 3.87 (tt, J = 16.4, 8.4 Hz, 2H), 3.66 (s, 1H), 3.44 (qd, J = 7.0, 5.1 |
| | Hz, 1H), 3.17 (d, J = 5.2 Hz, 2H), 3.00 (dt, J = 36.7, 9.6 Hz, 2H), 2.44- |
| | 2.16 (m, 4H), 2.11 (s, 2H), 1.96 (t, J = 9.9 Hz, 4H), 1.59 (dd, J = 15.1, |
| | 8.3 Hz, 1H), 1.37 (s, 9H), 0.49 (s, 9H). |
| 90 | ¹H NMR (400 MHz, DMSO-d₆) δ 13.05 (s, 1H), 8.42 (s, 1H), 7.91 (s, |
| | 1H), 7.65 (s, 2H), 7.24 (d, J = 7.9 Hz, 1H), 7.19-6.98 (m, 3H), 6.39 (s, |
| | 1H), 5.07 (dd, J = 10.7, 4.4 Hz, 1H), 4.33-4.23 (m, 1H), 3.85 (ddd, J = |
| | 32.0, 17.2, 8.9 Hz, 2H), 3.66 (s, 1H), 3.44 (qd, J = 7.0, 5.2 Hz, 1H), 3.17 |
| | (d, J = 5.3 Hz, 2H), 2.99 (dt, J = 18.8, 9.7 Hz, 2H), 2.39 (d, J = 10.9 Hz, |
| | 1H), 2.31-2.19 (m, 2H), 2.13 (s, 3H), 1.96-1.86 (m, 4H), 1.57 (dd, J = |
| | 15.1, 8.3 Hz, 1H), 1.37 (s, 9H), 0.48 (s, 9H). |
| 453 | ¹H NMR (400 MHz, DMSO-d₆) δ 13.04 (s, 1H), 8.35 (s, 1H), 7.88 (s, |
| | 1H), 7.66 (s, 2H), 7.25 (s, 1H), 7.12 (s, 2H), 6.38 (s, 1H), 5.04 (dd, J = |
| | 10.7, 4.5 Hz, 1H), 4.29 (t, J = 11.3 Hz, 1H), 4.10-4.00 (m, 1H), 4.00- |
| | 3.89 (m, 4H), 3.89-3.77 (m, 1H), 3.54 (s, 3H), 3.12 (t, J = 9.7 Hz, 1H), |
| | 3.04 (t, J = 9.9 Hz, 1H), 2.48-2.37 (m, 2H), 2.21-1.82 (m, 7H), 1.48 |
| | (dd, J = 16.6, 9.3 Hz, 1H), 0.88-0.70 (m, 2H), 0.69-0.58 (m, 1H), 0.58- |
| | 0.46 (m, 1H). |
| 118 | ¹H NMR (400 MHz, Methanol-d₄) δ 8.57 (d, J = 2.2 Hz, 1H), 8.09-8.00 |
| | (m, 1H), 7.78-7.67 (m, 2H), 7.30 (t, J = 7.6 Hz, 1H), 7.16 (d, J = 7.6 |
| | Hz, 2H), 6.30 (s, 1H), 5.29 (dd, J = 10.8, 4.3 Hz, 1H), 4.25 (t, J = 11.2 |
| | Hz, 1H), 4.06 (h, J = 8.5 Hz, 1H), 3.85 (ddd, J = 12.0, 8.0, 4.3 Hz, 1H), |
| | 3.79-3.66 (m, 2H), 3.28 (t, J = 9.7 Hz, 2H), 3.15 (dd, J = 11.4, 8.7 Hz, |
| | 1H), 2.69-2.49 (m, 3H), 2.43-2.33 (m, 1H), 2.29 (ddd, J = 11.6, 8.6, |
| | 2.8 Hz, 2H), 2.09 (s, 1H), 1.69 (dd, J = 15.3, 8.2 Hz, 1H), 1.54 (d, J = |
| | 15.1 Hz, 1H), 1.40-1.29 (m, 2H), 0.96-0.85 (m, 2H), 0.61 (s, 9H). |

### Example 157: Compounds 475-506

Compounds 475 to 506, depicted in Table 7, can be prepared following the procedures described above for compounds 1-474 and CFTR modulating activity can be assessed using one or more of the assays outlined below.

TABLE 7
Compounds 475 to 506

| Compound | |
|---|---|
| Number | Structure |
| 475 | 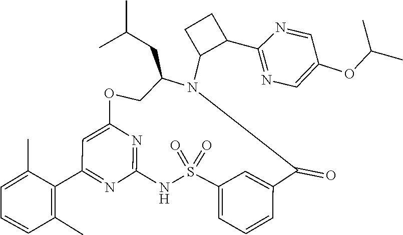 [SMILES: Cc1cccc(C)c1-c1cc2nc(n1)NS(=O)(=O)c1cccc(c1)C(=O)N(C1CCC1c1ncc(OC(C)C)cn1)[C@H](CC(C)C)CO2] |
| 476 | 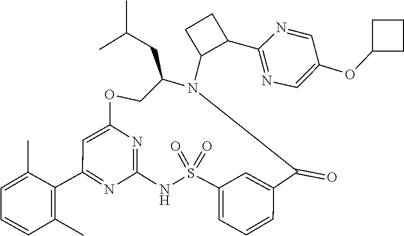 [SMILES: Cc1cccc(C)c1-c1cc2nc(n1)NS(=O)(=O)c1cccc(c1)C(=O)N(C1CCC1c1ncc(OC3CCC3)cn1)[C@H](CC(C)C)CO2] |
| 477 | 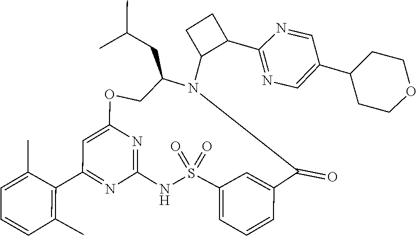 [SMILES: Cc1cccc(C)c1-c1cc2nc(n1)NS(=O)(=O)c1cccc(c1)C(=O)N(C1CCC1c1ncc(C3CCOCC3)cn1)[C@H](CC(C)C)CO2] |
| 478 | 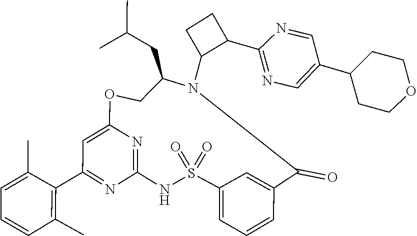 [SMILES: Cc1cccc(C)c1-c1cc2nc(n1)NS(=O)(=O)c1cccc(c1)C(=O)N(C1CCC1c1ncc(C3CCOCC3)cn1)[C@H](CC(C)C)CO2] |
| 479 | 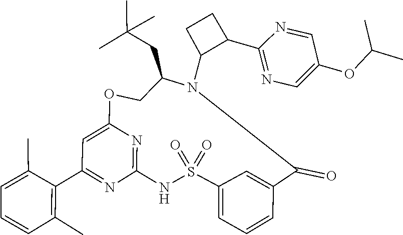 [SMILES: Cc1cccc(C)c1-c1cc2nc(n1)NS(=O)(=O)c1cccc(c1)C(=O)N(C1CCC1c1ncc(OC(C)C)cn1)[C@H](CC(C)(C)C)CO2] |
| 480 | 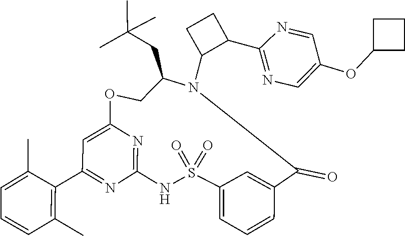 [SMILES: Cc1cccc(C)c1-c1cc2nc(n1)NS(=O)(=O)c1cccc(c1)C(=O)N(C1CCC1c1ncc(OC3CCC3)cn1)[C@H](CC(C)(C)C)CO2] |
| 481 | 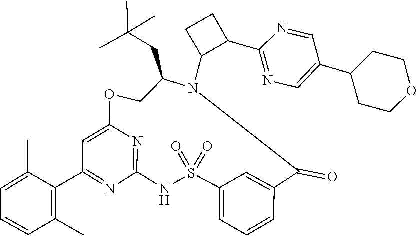 [SMILES: Cc1cccc(C)c1-c1cc2nc(n1)NS(=O)(=O)c1cccc(c1)C(=O)N(C1CCC1c1ncc(C3CCOCC3)cn1)[C@H](CC(C)(C)C)CO2] |
| 482 | 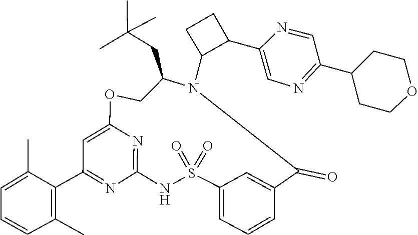 [SMILES: Cc1cccc(C)c1-c1cc2nc(n1)NS(=O)(=O)c1cccc(c1)C(=O)N(C1CCC1c1cnc(C3CCOCC3)cn1)[C@H](CC(C)(C)C)CO2] |
| 483 | 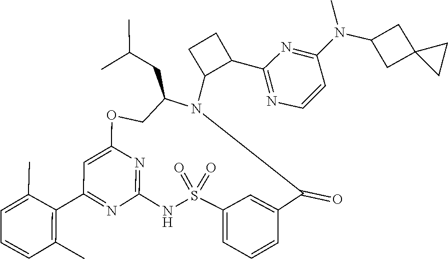 [SMILES: Cc1cccc(C)c1-c1cc2nc(n1)NS(=O)(=O)c1cccc(c1)C(=O)N(C1CCC1c1nccc(N(C)C3CC4(CC4)C3)n1)[C@H](CC(C)C)CO2] |
| 484 | 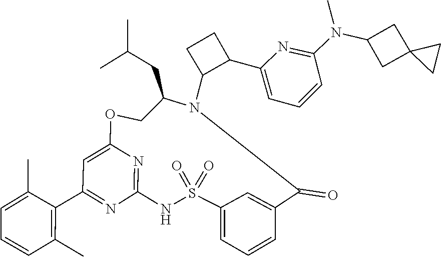 [SMILES: Cc1cccc(C)c1-c1cc2nc(n1)NS(=O)(=O)c1cccc(c1)C(=O)N(C1CCC1c1cccc(N(C)C3CC4(CC4)C3)n1)[C@H](CC(C)C)CO2] |
| 485 | 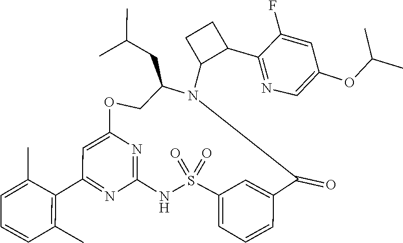 [SMILES: Cc1cccc(C)c1-c1cc2nc(n1)NS(=O)(=O)c1cccc(c1)C(=O)N(C1CCC1c1ncc(OC(C)C)cc1F)[C@H](CC(C)C)CO2] |
| 486 | 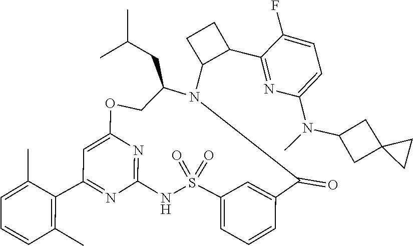 [SMILES: Cc1cccc(C)c1-c1cc2nc(n1)NS(=O)(=O)c1cccc(c1)C(=O)N(C1CCC1c1nc(N(C)C3CC4(CC4)C3)ccc1F)[C@H](CC(C)C)CO2] |
| 487 | 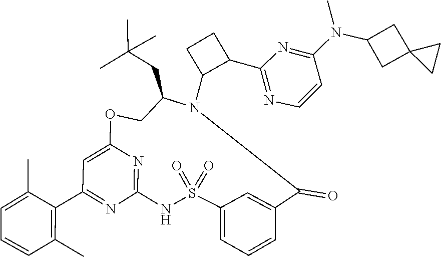 [SMILES: Cc1cccc(C)c1-c1cc2nc(n1)NS(=O)(=O)c1cccc(c1)C(=O)N(C1CCC1c1nccc(N(C)C3CC4(CC4)C3)n1)[C@H](CC(C)(C)C)CO2] |
| 488 | 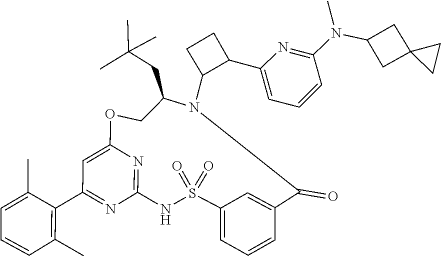 [SMILES: Cc1cccc(C)c1-c1cc2nc(n1)NS(=O)(=O)c1cccc(c1)C(=O)N(C1CCC1c1cccc(N(C)C3CC4(CC4)C3)n1)[C@H](CC(C)(C)C)CO2] |
| 489 | 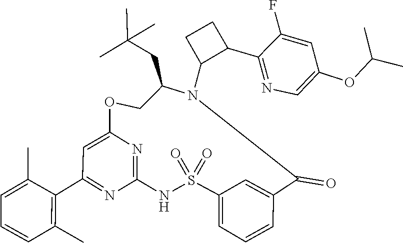 [SMILES: Cc1cccc(C)c1-c1cc2nc(n1)NS(=O)(=O)c1cccc(c1)C(=O)N(C1CCC1c1ncc(OC(C)C)cc1F)[C@H](CC(C)(C)C)CO2] |
| 490 | 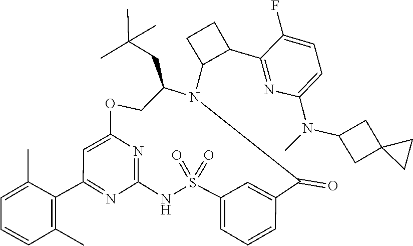 [SMILES: Cc1cccc(C)c1-c1cc2nc(n1)NS(=O)(=O)c1cccc(c1)C(=O)N(C1CCC1c1nc(N(C)C3CC4(CC4)C3)ccc1F)[C@H](CC(C)(C)C)CO2] |
| 491 | 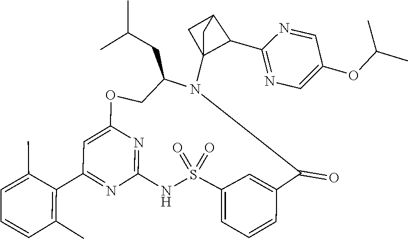 [SMILES: Cc1cccc(C)c1-c1cc2nc(n1)NS(=O)(=O)c1cccc(c1)C(=O)N(C13CC(C1)C3c1ncc(OC(C)C)cn1)[C@H](CC(C)C)CO2] |
| 492 | 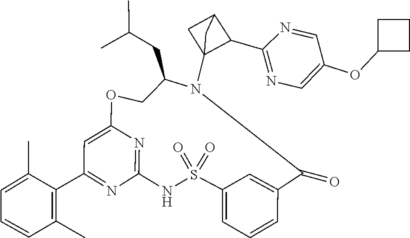 [SMILES: Cc1cccc(C)c1-c1cc2nc(n1)NS(=O)(=O)c1cccc(c1)C(=O)N(C13CC(C1)C3c1ncc(OC3CCC3)cn1)[C@H](CC(C)C)CO2] |
| 493 | 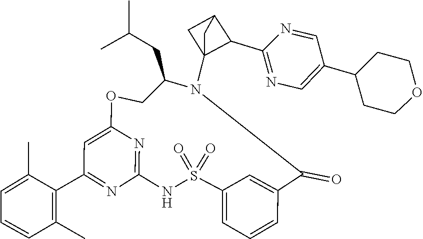 [SMILES: Cc1cccc(C)c1-c1cc2nc(n1)NS(=O)(=O)c1cccc(c1)C(=O)N(C13CC(C1)C3c1ncc(C3CCOCC3)cn1)[C@H](CC(C)C)CO2] |
| 494 | 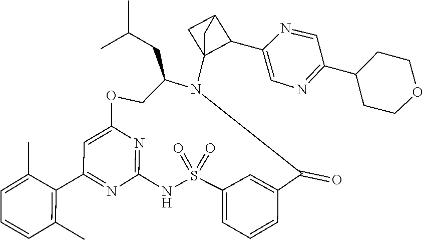 [SMILES: Cc1cccc(C)c1-c1cc2nc(n1)NS(=O)(=O)c1cccc(c1)C(=O)N(C13CC(C1)C3c1cnc(C3CCOCC3)cn1)[C@H](CC(C)C)CO2] |
| 495 | 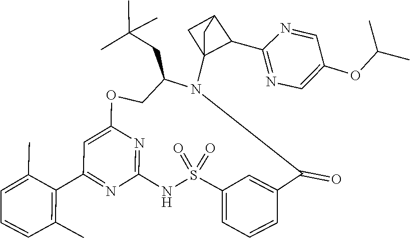 [SMILES: Cc1cccc(C)c1-c1cc2nc(n1)NS(=O)(=O)c1cccc(c1)C(=O)N(C13CC(C1)C3c1ncc(OC(C)C)cn1)[C@H](CC(C)(C)C)CO2] |
| 496 | 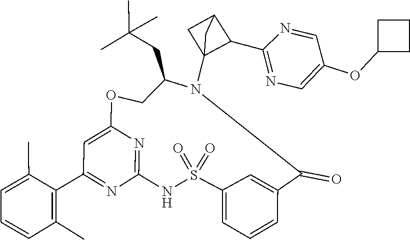 [SMILES: Cc1cccc(C)c1-c1cc2nc(n1)NS(=O)(=O)c1cccc(c1)C(=O)N(C13CC(C1)C3c1ncc(OC3CCC3)cn1)[C@H](CC(C)(C)C)CO2] |
| 497 | 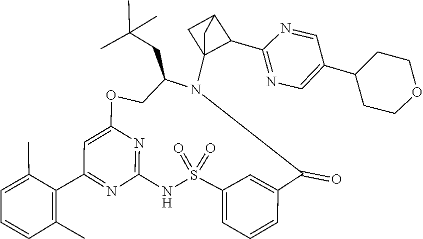 [SMILES: Cc1cccc(C)c1-c1cc2nc(n1)NS(=O)(=O)c1cccc(c1)C(=O)N(C13CC(C1)C3c1ncc(C3CCOCC3)cn1)[C@H](CC(C)(C)C)CO2] |
| 498 | 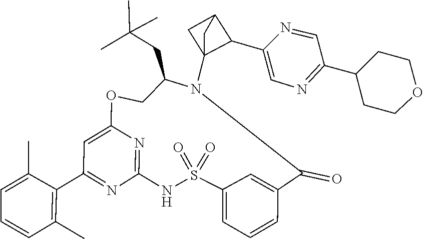 [SMILES: Cc1cccc(C)c1-c1cc2nc(n1)NS(=O)(=O)c1cccc(c1)C(=O)N(C13CC(C1)C3c1cnc(C3CCOCC3)cn1)[C@H](CC(C)(C)C)CO2] |
| 499 | 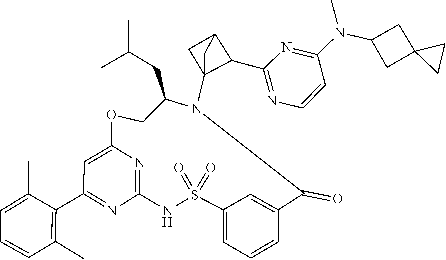 [SMILES: Cc1cccc(C)c1-c1cc2nc(n1)NS(=O)(=O)c1cccc(c1)C(=O)N(C13CC(C1)C3c1nccc(N(C)C3CC4(CC4)C3)n1)[C@H](CC(C)C)CO2] |
| 500 | 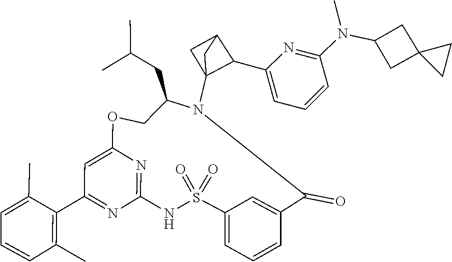 [SMILES: Cc1cccc(C)c1-c1cc2nc(n1)NS(=O)(=O)c1cccc(c1)C(=O)N(C13CC(C1)C3c1cccc(N(C)C3CC4(CC4)C3)n1)[C@H](CC(C)C)CO2] |
| 501 | 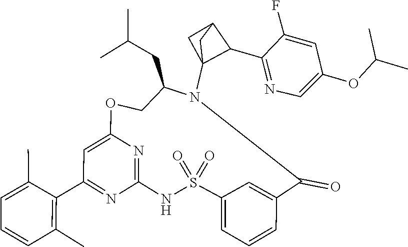 [SMILES: Cc1cccc(C)c1-c1cc2nc(n1)NS(=O)(=O)c1cccc(c1)C(=O)N(C13CC(C1)C3c1ncc(OC(C)C)cc1F)[C@H](CC(C)C)CO2] |
| 502 | 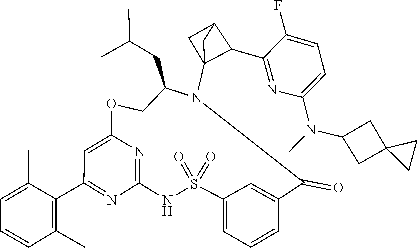 [SMILES: Cc1cccc(C)c1-c1cc2nc(n1)NS(=O)(=O)c1cccc(c1)C(=O)N(C13CC(C1)C3c1nc(N(C)C3CC4(CC4)C3)ccc1F)[C@H](CC(C)C)CO2] |
| 503 | 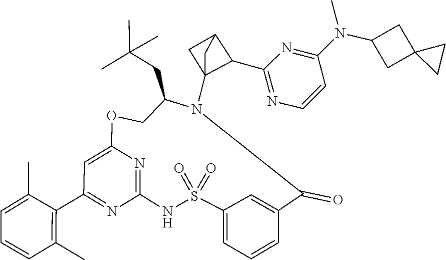 [SMILES: Cc1cccc(C)c1-c1cc2nc(n1)NS(=O)(=O)c1cccc(c1)C(=O)N(C13CC(C1)C3c1nccc(N(C)C3CC4(CC4)C3)n1)[C@H](CC(C)(C)C)CO2] |
| 504 | 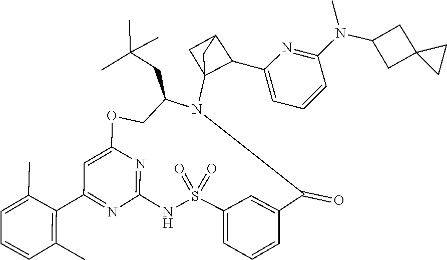 [SMILES: Cc1cccc(C)c1-c1cc2nc(n1)NS(=O)(=O)c1cccc(c1)C(=O)N(C13CC(C1)C3c1cccc(N(C)C3CC4(CC4)C3)n1)[C@H](CC(C)(C)C)CO2] |
| 505 | 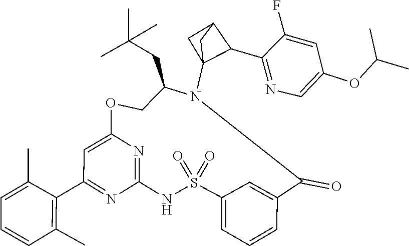 [SMILES: Cc1cccc(C)c1-c1cc2nc(n1)NS(=O)(=O)c1cccc(c1)C(=O)N(C13CC(C1)C3c1ncc(OC(C)C)cc1F)[C@H](CC(C)(C)C)CO2] |
| 506 | 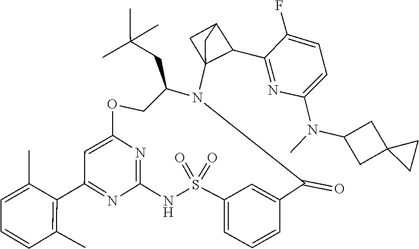 [SMILES: Cc1cccc(C)c1-c1cc2nc(n1)NS(=O)(=O)c1cccc(c1)C(=O)N(C13CC(C1)C3c1nc(N(C)C3CC4(CC4)C3)ccc1F)[C@H](CC(C)(C)C)CO2] |

### VII. Bioactivity Data

### A. Enteroid Assay

1. Solutions

Base medium (ADF+++) consisted of Advanced DMEM/4Ham's F12, 2 mM Glutamax, 10 mM HEPES, 1 μg/mL penicillin/streptomycin.

Intestinal enteroid maintenance medium (IEMMN) consisted of ADF+++, 1× B27 supplement, 1× N2 supplement, 1.25 mM N-acetyl cysteine, 10 mM Nicotinamide, 50 ng/mL hEGF, 10 nM Gastrin, 1 μg/mL hR-spondin-1, 100 ng/mL hNoggin, TGF-b type 1 inhibitor A-83-01, 100 μg/mL Primocin, 10 μM P38 MAPK inhibitor SB202190.

Bath 1 Buffer consisted of 1 mM MgCl₂, 160 mM NaCl, 4.5 mM KCl, 10 mM HEPES, 10 mM Glucose, 2 mM CaCl₂).

Chloride Free Buffer consisted of 1 mM Magnesium Gluconate, 2 mM Calcium Gluconate, 4.5 mM Potassium Gluconate, 160 mM Sodium Gluconate, 10 mM HEPES, 10 mM Glucose.

Bath 1 Dye Solution consisted of Bath 1 Buffer, 0.04% Pluronic F127, 20 μM Methyl Oxonol, 30 μM CaCCinh-A01, 30 μM Chicago Sky Blue.

Chloride Free Dye Solution consisted of Chloride Free Buffer, 0.04% Pluronic F127, 20 μM Methyl Oxonol, 30 μM CaCCinh-A01, 30 μM Chicago Sky Blue.

Chloride Free Dye Stimulation Solution consisted of Chloride Free Dye Solution, 10 μM forskolin, 100 μM IBMX, and 300 nM Compound III.

2. Cell Culture

Human intestinal epithelial enteroid cells were obtained from the Hubrecht Institute for Developmental Biology and Stem Cell Research, Utrecht, The Netherlands and expanded in T-Flasks as previously described (Dekkers J F, Wiegerinck C L, de Jonge H R, Bronsveld I, Janssens H M, de Winter-de Groot K M, Brandsma A M, de Jong N W M, Bijvelds M J C, Scholte B J, Nieuwenhuis E E S, van den Brink S, Clevers H, van der Ent C K, Middendorp S and M Beekman J M. A functional CFTR assay using primary cystic fibrosis intestinal organoids. Nat Med. 2013 July; 19(7):939-45.).

3. Enteroid Cell Harvesting and Seeding

Cells were recovered in cell recovery solution, collected by centrifugation at 650 rpm for 5 min at 4° C., resuspended in TrypLE and incubated for 5 min at 37° C. Cells were then collected by centrifugation at 650 rpm for 5 min at 4° C. and resuspended in IEMM containing 10 μM ROCK inhibitor (RI). The cell suspension was passed through a 40 μm cell strainer and resuspended at 1×106 cells/mL in IEMM containing 10 μM RI. Cells were seeded at 5000 cells/well into multi-well plates and incubated for overnight at 37° C., 95% humidity and 5% CO₂ prior to assay.

4. Membrane Potential Dye, Enteroid Assay A

Enteroid cells were incubated with test compound in IEMM for 18-24 hours at 37° C., 95% humidity and 5% CO₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added, and the cells were incubated for 25 min at room temperature. Following dye incubation, cells were washed 3 times in Chloride Free Dye Solution. Chloride transport was initiated by addition of Chloride Free Dye Stimulation Solution and the fluorescence signal was read for 15 min. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 3 μM N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity).

5. Membrane Potential Dye, Enteroid Assay B

Enteroid cells were incubated with test compound in IEMM for 18-24 hours at 37° C., 95% humidity and 5% CO₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added and the cells were incubated for 25 min at room temperature. Following dye incubation, cells were washed 3 times in Chloride Free Dye Solution. Chloride transport was initiated by addition of Chloride Free Dye Stimulation Solution and the fluorescence signal was read for 15 min. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 1 μM (14S)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity).

### B. HBE assay

1. Ussing Chamber Assay of CFTR-mediated short-circuit currents

Ussing chamber experiments were performed using human bronchial epithelial (HBE) cells derived from CF subjects heterozygous for F508del and a minimal function CFTR mutation (F508del/MF-HBE) and cultured as previously described (Neuberger T, Burton B, Clark H, Van Goor F Methods Mol Biol 2011:741:39-54). After four days the apical media was removed, and the cells were grown at an air liquid interface for >14 days prior to use. This resulted in a monolayer of fully differentiated columnar cells that were ciliated, features that are characteristic of human bronchial airway epithelia.

To isolate the CFTR-mediated short-circuit (I_{SC}) current, F508del/MF-HBE grown on Costar® Snapwell™ cell culture inserts were mounted in an Ussing chamber and the transepithelial I_{SC} was measured under voltage-clamp recording conditions (V_{hold}=0 mV) at 37° C. The basolateral solution contained (in mM) 145 NaCl, 0.83 K₂HPO₄, 3.3 KH₂PO₄, 1.2 MgCl₂, 1.2 CaCl₂), 10 Glucose, 10 HEPES (pH adjusted to 7.4 with NaOH) and the apical solution contained (in mM) 145 NaGluconate, 1.2 MgCl₂, 1.2 CaCl₂), 10 glucose, 10 HEPES (pH adjusted to 7.4 with NaOH) and 30 μM amiloride to block the epithelial sodium channel. Forskolin (20 μM) was added to the apical surface to activate CFTR, followed by apical addition of a CFTR inhibitor cocktail consisting of BPO, GlyH-101, and CFTR inhibitor 172 (each at 20 μM final assay concentration) to specifically isolate CFTR currents. The CFTR-mediated I_{SC} (μA/cm²) for each condition was determined from the peak forskolin response to the steady-state current following inhibition.

2. Identification of Corrector Compounds

The activity of the CFTR corrector compounds on the CFTR-mediated I_{SC} was determined in Ussing chamber studies as described above. The F508del/MF-HBE cell cultures were either incubated with the corrector compounds at a range of concentrations in combination with 1 μM Ivacaftor or were incubated with the corrector compounds at a single fixed concentration of 10 μM in combination with 1 μM Ivacaftor for 18-24 hours at 37° C. and in the presence of 20% human serum. The concentration of corrector compounds with 1 μM Ivacaftor during the 18-24 hours incubations was kept constant throughout the Ussing chamber measurement of the CFTR-mediated I_{SC} to ensure compounds were present throughout the entire experiment. The efficacy and potency of the putative F508del correctors was compared to that of the known Vertex corrector, (14S′)-8-[3-(2-{Dispiro[2.0.2.1]heptan-7-yl}ethoxy)-1H-pyrazol -1-yl]-12 ,12-dimethyl-2λ⁶-thia-3,9,11,18,23-pentaazatetracyclo[17.3.1.111,14.05,10]tetracosa-1(22),5,7,9,19(23),20-hexaene-2,2,4-tri one, in combination with 18 μM Tezacaftor and 1 μM Ivacaftor.

### C. Biological Activity Data Tables

Table 8 represents CFTR modulating activity for representative compounds of the disclosure generated using one or more of the assays described in this example (EC50: +++ is <1 μM; ++ is 1-<3 μM; + is 3-<30 μM; and ND is “not detected in this assay.” 0% Activity: +++ is >60%; ++ is 30-60%; + is <300%).

TABLE 8
Biological Activity Data

| | | | Ent. A | | Ent. B |
|---|---|---|---|---|---|
| | | Ent. A | Max | Ent. B | Max |
| Cmpd | | EC₅₀ | Activity | EC₅₀ | Activity |
| No. | Structure | (μM) | (%) | (μM) | (%) |
| 439 | 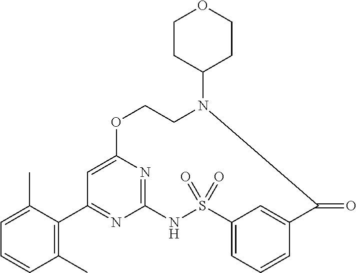 [SMILES: Cc1cccc(C)c1-c1cc2nc(n1)NS(=O)(=O)c1cccc(c1)C(=O)N(C1CCOCC1)CCO2] | + | +++ | | |
| 438 | 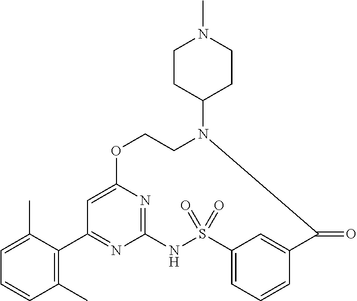 [SMILES: Cc1cccc(C)c1-c1cc2nc(n1)NS(=O)(=O)c1cccc(c1)C(=O)N(C1CCN(C)CC1)CCO2] | ND | + | | |
| 27 | 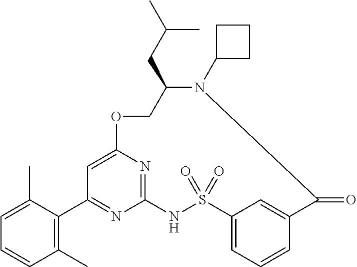 [SMILES: Cc1cccc(C)c1-c1cc2nc(n1)NS(=O)(=O)c1cccc(c1)C(=O)N(C1CCC1)[C@H](CC(C)C)CO2] | +++ | ++ | | |
| 28 | 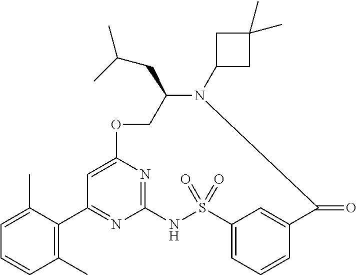 [SMILES: Cc1cccc(C)c1-c1cc2nc(n1)NS(=O)(=O)c1cccc(c1)C(=O)N(C1CC(C)(C)C1)[C@H](CC(C)C)CO2] | +++ | +++ | | |
| 29 | 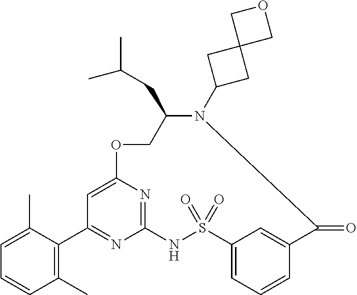 [SMILES: Cc1cccc(C)c1-c1cc2nc(n1)NS(=O)(=O)c1cccc(c1)C(=O)N(C1CC3(COC3)C1)[C@H](CC(C)C)CO2] | +++ | +++ | | |
| 30 | 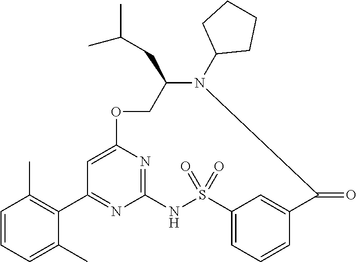 [SMILES: Cc1cccc(C)c1-c1cc2nc(n1)NS(=O)(=O)c1cccc(c1)C(=O)N(C1CCCC1)[C@H](CC(C)C)CO2] | +++ | +++ | | |
| 42 | 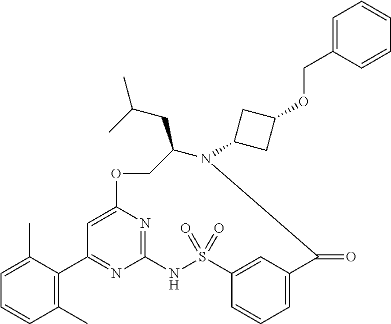 [SMILES: Cc1cccc(C)c1-c1cc2nc(n1)NS(=O)(=O)c1cccc(c1)C(=O)N([C@H]1C[C@@H](OCc3ccccc3)C1)[C@H](CC(C)C)CO2] | +++ | ++ | | |
| 348 | 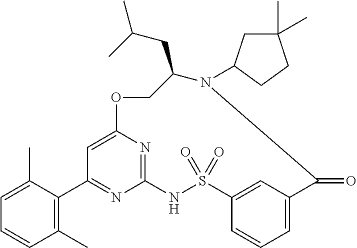 [SMILES: Cc1cccc(C)c1-c1cc2nc(n1)NS(=O)(=O)c1cccc(c1)C(=O)N(C1CCC(C)(C)C1)[C@H](CC(C)C)CO2] | +++ | +++ | | |
| 43 | 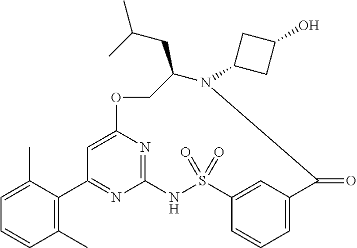 [SMILES: Cc1cccc(C)c1-c1cc2nc(n1)NS(=O)(=O)c1cccc(c1)C(=O)N([C@H]1C[C@@H](O)C1)[C@H](CC(C)C)CO2] | ++ | +++ | | |
| 44 | 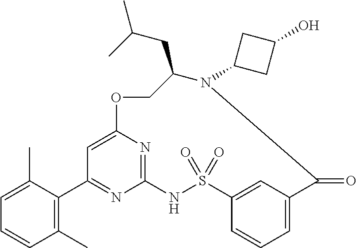 [SMILES: Cc1cccc(C)c1-c1cc2nc(n1)NS(=O)(=O)c1cccc(c1)C(=O)N([C@H]1C[C@@H](O)C1)[C@H](CC(C)C)CO2] | +++ | +++ | | |
| 126 | 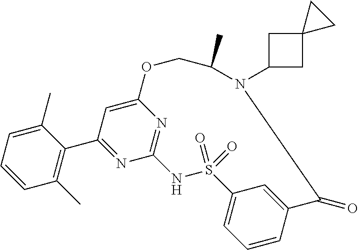 [SMILES: Cc1cccc(C)c1-c1cc2nc(n1)NS(=O)(=O)c1cccc(c1)C(=O)N(C1CC3(CC3)C1)[C@H](C)CO2] | ++ | ++ | | |
| 40 | 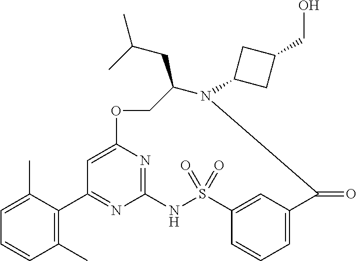 [SMILES: Cc1cccc(C)c1-c1cc2nc(n1)NS(=O)(=O)c1cccc(c1)C(=O)N([C@H]1C[C@@H](CO)C1)[C@H](CC(C)C)CO2] | ++ | +++ | | |
| 13 | 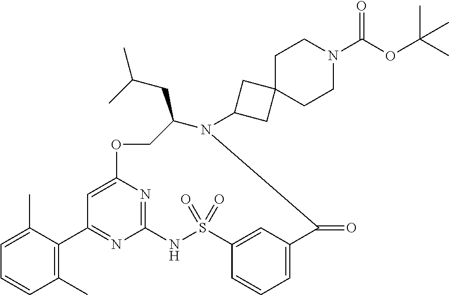 [SMILES: Cc1cccc(C)c1-c1cc2nc(n1)NS(=O)(=O)c1cccc(c1)C(=O)N(C1CC3(CCN(C(=O)OC(C)(C)C)CC3)C1)[C@H](CC(C)C)CO2] | +++ | +++ | +++ | +++ |
| 14 | 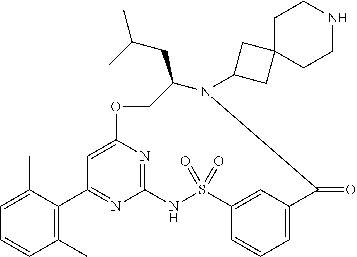 [SMILES: Cc1cccc(C)c1-c1cc2nc(n1)NS(=O)(=O)c1cccc(c1)C(=O)N(C1CC3(CCNCC3)C1)[C@H](CC(C)C)CO2] | + | +++ | +++ | +++ |
| 15 | 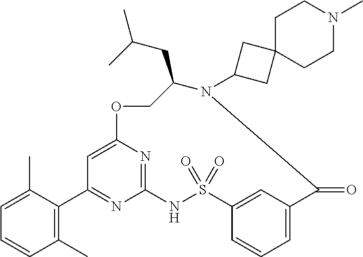 [SMILES: Cc1cccc(C)c1-c1cc2nc(n1)NS(=O)(=O)c1cccc(c1)C(=O)N(C1CC3(CCN(C)CC3)C1)[C@H](CC(C)C)CO2] | +++ | +++ | +++ | +++ |
| 349 | 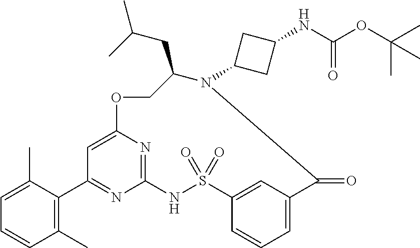 [SMILES: Cc1cccc(C)c1-c1cc2nc(n1)NS(=O)(=O)c1cccc(c1)C(=O)N([C@H]1C[C@@H](NC(=O)OC(C)(C)C)C1)[C@H](CC(C)C)CO2] | +++ | +++ | +++ | ++ |
| 351 | 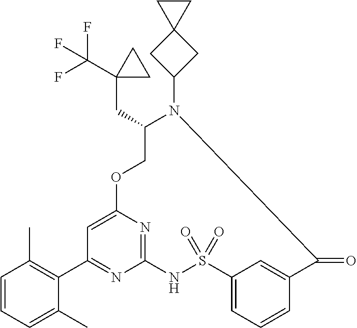 [SMILES: Cc1cccc(C)c1-c1cc2nc(n1)NS(=O)(=O)c1cccc(c1)C(=O)N(C1CC3(CC3)C1)[C@@H](CC1(C(F)(F)F)CC1)CO2] | +++ | + | | |
| 198 | 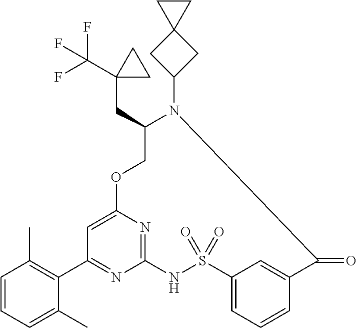 [SMILES: Cc1cccc(C)c1-c1cc2nc(n1)NS(=O)(=O)c1cccc(c1)C(=O)N(C1CC3(CC3)C1)[C@H](CC1(C(F)(F)F)CC1)CO2] | +++ | +++ | +++ | +++ |
| 16 | 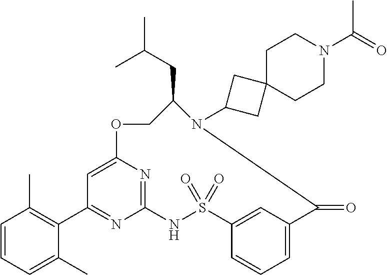 [SMILES: CC(=O)N1CCC2(CC1)CC(N1C(=O)c3cccc(c3)S(=O)(=O)Nc3nc(cc(-c4c(C)cccc4C)n3)OC[C@H]1CC(C)C)C2] | +++ | +++ | +++ | +++ |
| 17 | 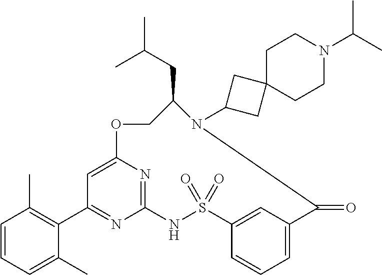 [SMILES: Cc1cccc(C)c1-c1cc2nc(n1)NS(=O)(=O)c1cccc(c1)C(=O)N(C1CC3(CCN(C(C)C)CC3)C1)[C@H](CC(C)C)CO2] | +++ | +++ | | |
| 346 | 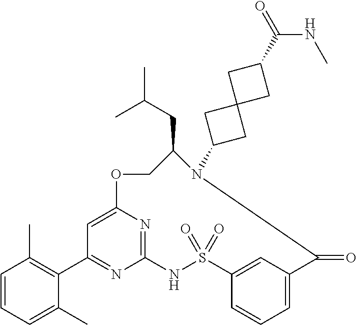 [SMILES: CNC(=O)[C@H]1CC2(C1)C[C@H](N1C(=O)c3cccc(c3)S(=O)(=O)Nc3nc(cc(-c4c(C)cccc4C)n3)OC[C@H]1CC(C)C)C2] | +++ | +++ | +++ | +++ |
| 345 | 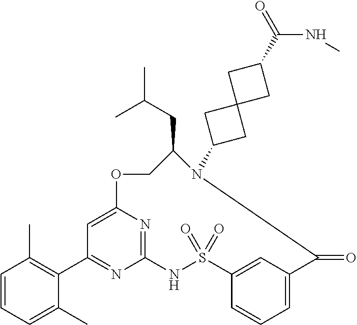 [SMILES: CNC(=O)[C@H]1CC2(C1)C[C@H](N1C(=O)c3cccc(c3)S(=O)(=O)Nc3nc(cc(-c4c(C)cccc4C)n3)OC[C@H]1CC(C)C)C2] | +++ | +++ | +++ | +++ |
| 344 | 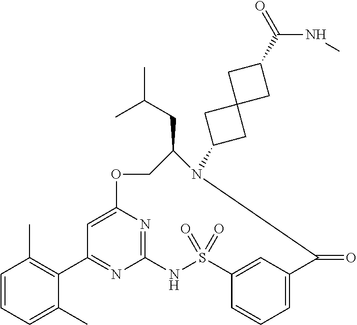 [SMILES: CNC(=O)[C@H]1CC2(C1)C[C@H](N1C(=O)c3cccc(c3)S(=O)(=O)Nc3nc(cc(-c4c(C)cccc4C)n3)OC[C@H]1CC(C)C)C2] | +++ | +++ | +++ | +++ |
| 343 | 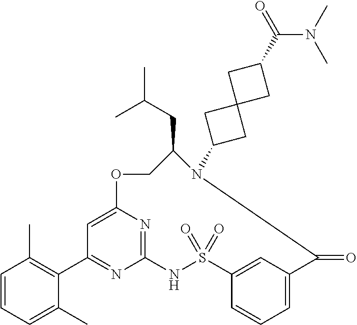 [SMILES: Cc1cccc(C)c1-c1cc2nc(n1)NS(=O)(=O)c1cccc(c1)C(=O)N([C@H]1CC3(C[C@H](C(=O)N(C)C)C3)C1)[C@H](CC(C)C)CO2] | +++ | +++ | +++ | +++ |
| 342 | 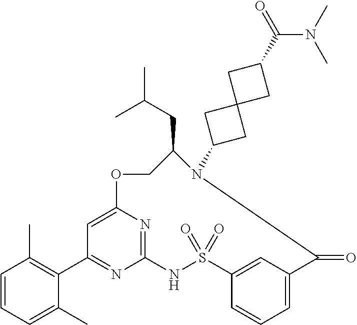 [SMILES: Cc1cccc(C)c1-c1cc2nc(n1)NS(=O)(=O)c1cccc(c1)C(=O)N([C@H]1CC3(C[C@H](C(=O)N(C)C)C3)C1)[C@H](CC(C)C)CO2] | +++ | +++ | +++ | +++ |
| 347 | 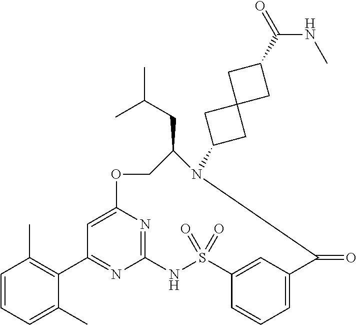 [SMILES: CNC(=O)[C@H]1CC2(C1)C[C@H](N1C(=O)c3cccc(c3)S(=O)(=O)Nc3nc(cc(-c4c(C)cccc4C)n3)OC[C@H]1CC(C)C)C2] | +++ | +++ | +++ | +++ |
| 136 | 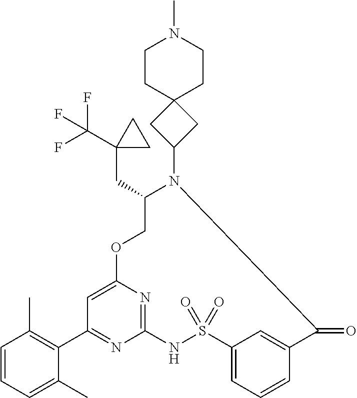 [SMILES: Cc1cccc(C)c1-c1cc2nc(n1)NS(=O)(=O)c1cccc(c1)C(=O)N(C1CC3(CCN(C)CC3)C1)[C@@H](CC1(C(F)(F)F)CC1)CO2] | +++ | +++ | | |
| 137 | 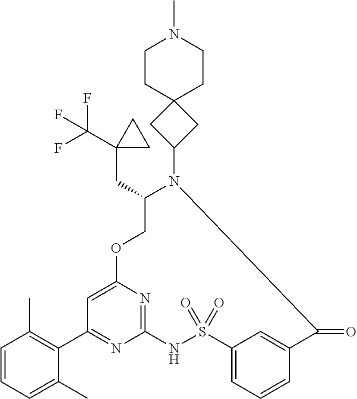 [SMILES: Cc1cccc(C)c1-c1cc2nc(n1)NS(=O)(=O)c1cccc(c1)C(=O)N(C1CC3(CCN(C)CC3)C1)[C@@H](CC1(C(F)(F)F)CC1)CO2] | ++ | +++ | | |
| 341 | 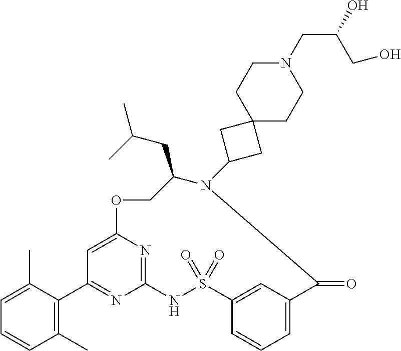 [SMILES: Cc1cccc(C)c1-c1cc2nc(n1)NS(=O)(=O)c1cccc(c1)C(=O)N(C1CC3(CCN(C[C@H](O)CO)CC3)C1)[C@H](CC(C)C)CO2] | +++ | +++ | | |
| 340 | 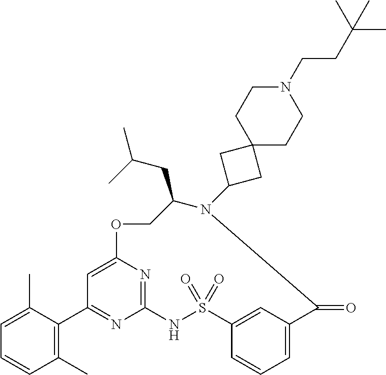 [SMILES: Cc1cccc(C)c1-c1cc2nc(n1)NS(=O)(=O)c1cccc(c1)C(=O)N(C1CC3(CCN(CCC(C)(C)C)CC3)C1)[C@H](CC(C)C)CO2] | +++ | +++ | | |
| 19 | 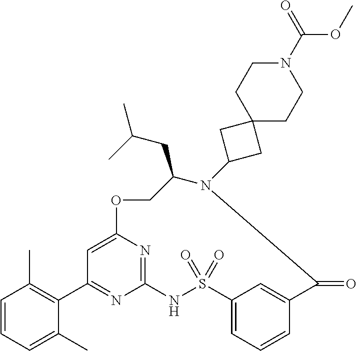 [SMILES: COC(=O)N1CCC2(CC1)CC(N1C(=O)c3cccc(c3)S(=O)(=O)Nc3nc(cc(-c4c(C)cccc4C)n3)OC[C@H]1CC(C)C)C2] | +++ | +++ | +++ | +++ |
| 339 | 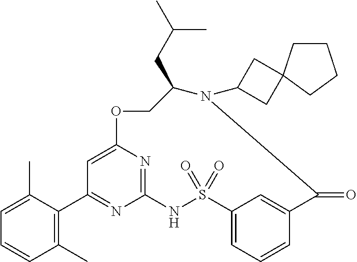 [SMILES: Cc1cccc(C)c1-c1cc2nc(n1)NS(=O)(=O)c1cccc(c1)C(=O)N(C1CC3(CCCC3)C1)[C@H](CC(C)C)CO2] | +++ | +++ | | |
| 338 | 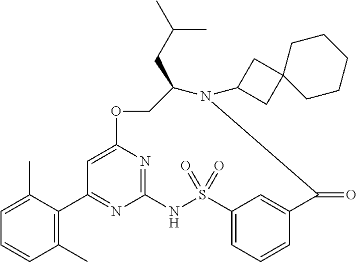 [SMILES: Cc1cccc(C)c1-c1cc2nc(n1)NS(=O)(=O)c1cccc(c1)C(=O)N(C1CC3(CCCCC3)C1)[C@H](CC(C)C)CO2] | +++ | ++ | | |
| 474 | 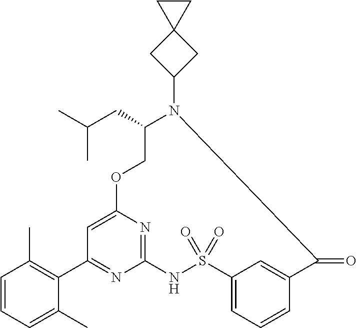 [SMILES: Cc1cccc(C)c1-c1cc2nc(n1)NS(=O)(=O)c1cccc(c1)C(=O)N(C1CC3(CC3)C1)[C@@H](CC(C)C)CO2] | +++ | ++ | +++ | + |
| 31 | 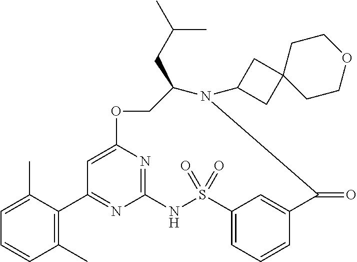 [SMILES: Cc1cccc(C)c1-c1cc2nc(n1)NS(=O)(=O)c1cccc(c1)C(=O)N(C1CC3(CCOCC3)C1)[C@H](CC(C)C)CO2] | +++ | +++ | | |
| 32 | 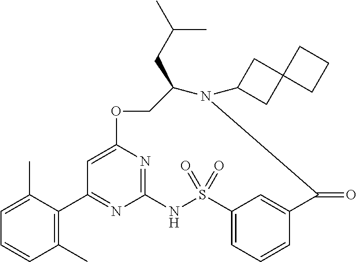 [SMILES: Cc1cccc(C)c1-c1cc2nc(n1)NS(=O)(=O)c1cccc(c1)C(=O)N(C1CC3(CCC3)C1)[C@H](CC(C)C)CO2] | +++ | ++ | | |
| 109 | 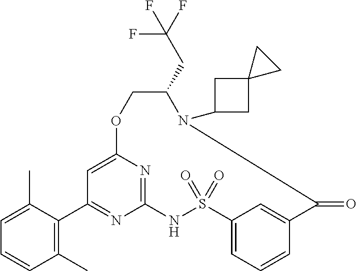 [SMILES: Cc1cccc(C)c1-c1cc2nc(n1)NS(=O)(=O)c1cccc(c1)C(=O)N(C1CC3(CC3)C1)[C@@H](CC(F)(F)F)CO2] | ++ | ++ | | |
| 125 | 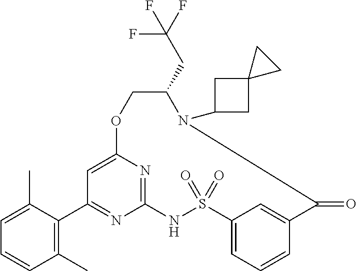 [SMILES: Cc1cccc(C)c1-c1cc2nc(n1)NS(=O)(=O)c1cccc(c1)C(=O)N(C1CC3(CC3)C1)[C@@H](CC(F)(F)F)CO2] | +++ | ++ | | |
| 337 | 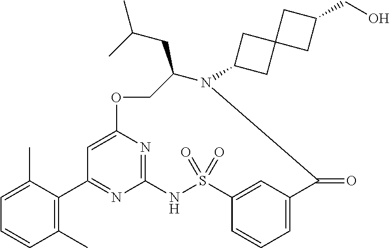 [SMILES: Cc1cccc(C)c1-c1cc2nc(n1)NS(=O)(=O)c1cccc(c1)C(=O)N([C@H]1CC3(C[C@@H](CO)C3)C1)[C@H](CC(C)C)CO2] | +++ | +++ | +++ | +++ |
| 336 | 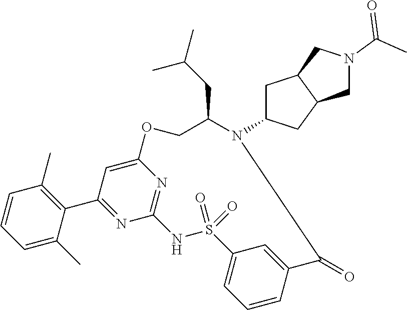 [SMILES: CC(=O)N1C[C@H]2C[C@H](N3C(=O)c4cccc(c4)S(=O)(=O)Nc4nc(cc(-c5c(C)cccc5C)n4)OC[C@H]3CC(C)C)C[C@H]2C1] | +++ | ++ | | |
| 335 | 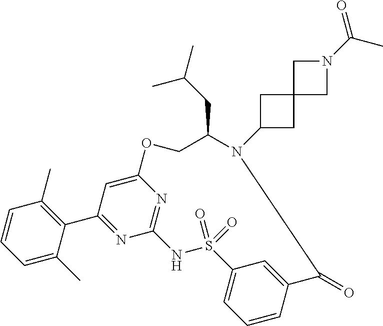 [SMILES: CC(=O)N1CC2(CC(N3C(=O)c4cccc(c4)S(=O)(=O)Nc4nc(cc(-c5c(C)cccc5C)n4)OC[C@H]3CC(C)C)C2)C1] | +++ | ++ | | |
| 3 | 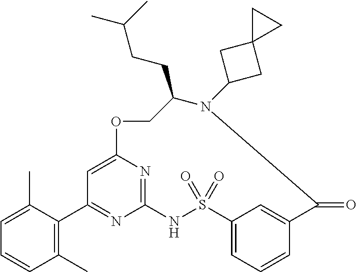 [SMILES: Cc1cccc(C)c1-c1cc2nc(n1)NS(=O)(=O)c1cccc(c1)C(=O)N(C1CC3(CC3)C1)[C@H](CCC(C)C)CO2] | +++ | ++ | | |
| 216 | 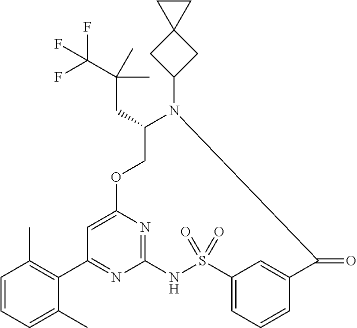 [SMILES: Cc1cccc(C)c1-c1cc2nc(n1)NS(=O)(=O)c1cccc(c1)C(=O)N(C1CC3(CC3)C1)[C@@H](CC(C)(C)C(F)(F)F)CO2] | +++ | +++ | | |
| 217 | 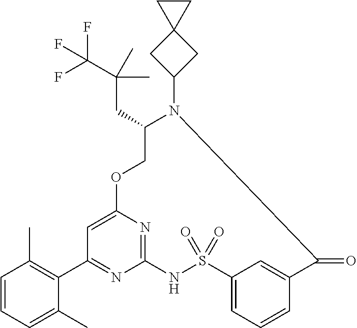 [SMILES: Cc1cccc(C)c1-c1cc2nc(n1)NS(=O)(=O)c1cccc(c1)C(=O)N(C1CC3(CC3)C1)[C@@H](CC(C)(C)C(F)(F)F)CO2] | | | +++ | +++ |
| 12 | 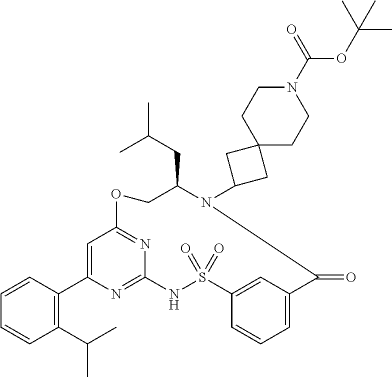 [SMILES: CC(C)C[C@@H]1COc2cc(-c3ccccc3C(C)C)nc(n2)NS(=O)(=O)c2cccc(c2)C(=O)N1C1CC2(CCN(C(=O)OC(C)(C)C)CC2)C1] | +++ | ++ | | |
| 11 | 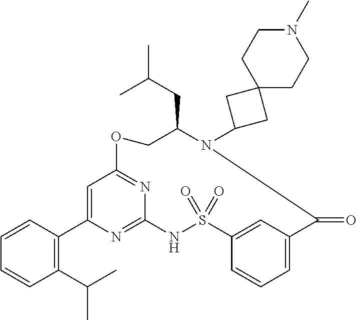 [SMILES: CC(C)C[C@@H]1COc2cc(-c3ccccc3C(C)C)nc(n2)NS(=O)(=O)c2cccc(c2)C(=O)N1C1CC2(CCN(C)CC2)C1] | +++ | +++ | | |
| 1 | 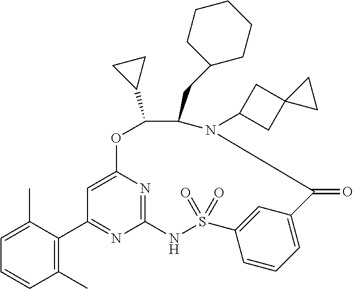 [SMILES: Cc1cccc(C)c1-c1cc2nc(n1)NS(=O)(=O)c1cccc(c1)C(=O)N(C1CC3(CC3)C1)[C@H](CC1CCCCC1)[C@@H](C1CC1)O2] | +++ | +++ | | |
| 471 | 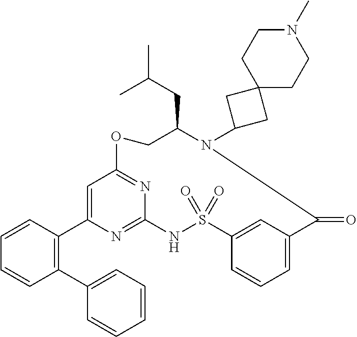 [SMILES: CC(C)C[C@@H]1COc2cc(-c3ccccc3-c3ccccc3)nc(n2)NS(=O)(=O)c2cccc(c2)C(=O)N1C1CC2(CCN(C)CC2)C1] | +++ | +++ | | |
| 334 | 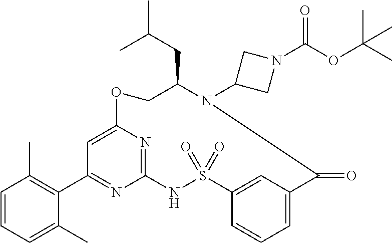 [SMILES: Cc1cccc(C)c1-c1cc2nc(n1)NS(=O)(=O)c1cccc(c1)C(=O)N(C1CN(C(=O)OC(C)(C)C)C1)[C@H](CC(C)C)CO2] | +++ | +++ | +++ | ++ |
| 333 | 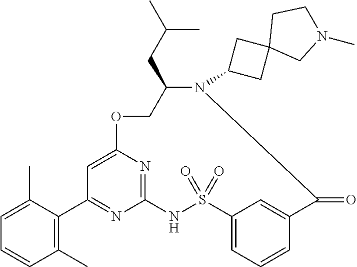 | +++ | +++ | | |
| 330 | 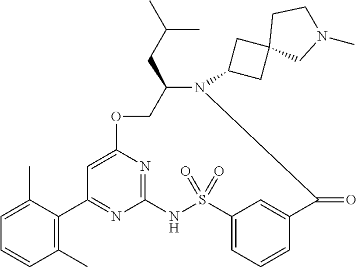 [SMILES: Cc1cccc(C)c1-c1cc2nc(n1)NS(=O)(=O)c1cccc(c1)C(=O)N([C@H]1C[C@]3(CCN(C)C3)C1)[C@H](CC(C)C)CO2] | +++ | +++ | | |
| 332 | 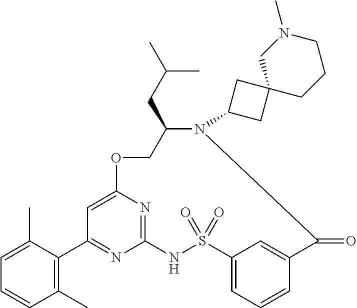 [SMILES: Cc1cccc(C)c1-c1cc2nc(n1)NS(=O)(=O)c1cccc(c1)C(=O)N([C@H]1C[C@]3(CCCN(C)C3)C1)[C@H](CC(C)C)CO2] | +++ | +++ | | |
| 329 | 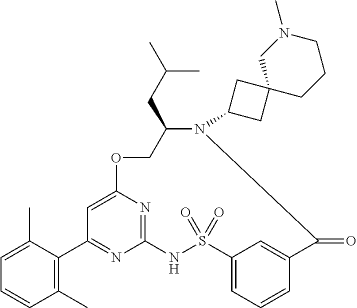 [SMILES: Cc1cccc(C)c1-c1cc2nc(n1)NS(=O)(=O)c1cccc(c1)C(=O)N([C@H]1C[C@]3(CCCN(C)C3)C1)[C@H](CC(C)C)CO2] | | | | |
| 331 | 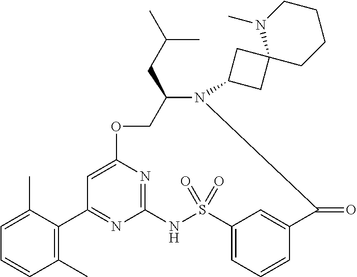 [SMILES: Cc1cccc(C)c1-c1cc2nc(n1)NS(=O)(=O)c1cccc(c1)C(=O)N([C@H]1C[C@]3(CCCCN3C)C1)[C@H](CC(C)C)CO2] | +++ | +++ | | |
| 18 | 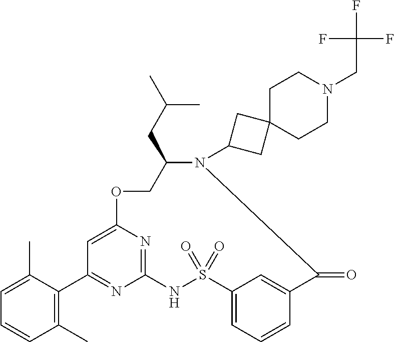 [SMILES: Cc1cccc(C)c1-c1cc2nc(n1)NS(=O)(=O)c1cccc(c1)C(=O)N(C1CC3(CCN(CC(F)(F)F)CC3)C1)[C@H](CC(C)C)CO2] | +++ | +++ | +++ | +++ |
| 326 | 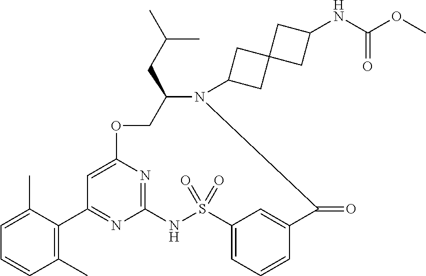 [SMILES: COC(=O)NC1CC2(C1)CC(N1C(=O)c3cccc(c3)S(=O)(=O)Nc3nc(cc(-c4c(C)cccc4C)n3)OC[C@H]1CC(C)C)C2] | +++ | +++ | +++ | +++ |
| 325 | 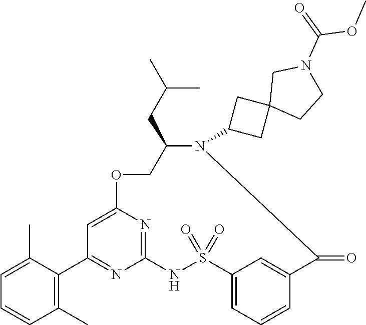 | +++ | +++ | | |
| 327 | 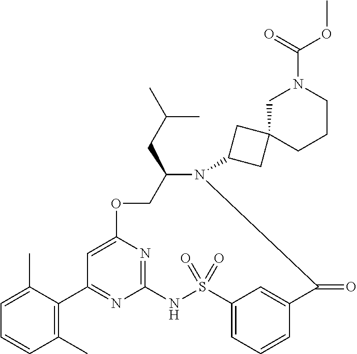 | +++ | +++ | | |
| 324 | 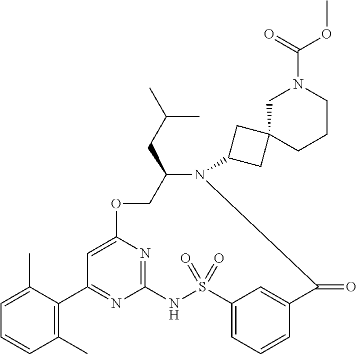 | +++ | +++ | | |
| 323 | 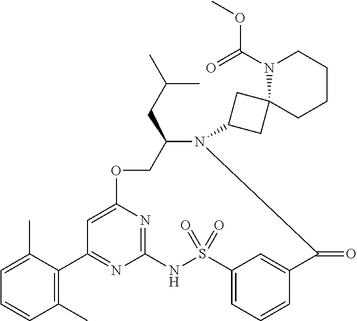 | +++ | +++ | +++ | +++ |
| 328 | 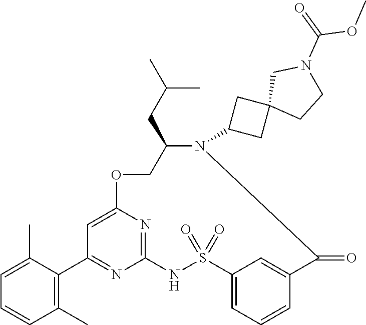 | +++ | +++ | | |
| 100 | 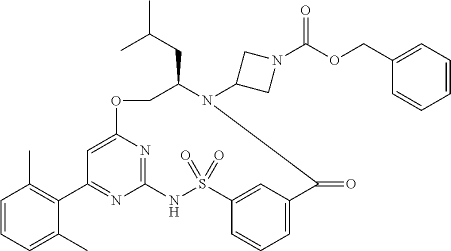 [SMILES: Cc1cccc(C)c1-c1cc2nc(n1)NS(=O)(=O)c1cccc(c1)C(=O)N(C1CN(C(=O)OCc3ccccc3)C1)[C@H](CC(C)C)CO2] | +++ | +++ | +++ | +++ |
| 322 | 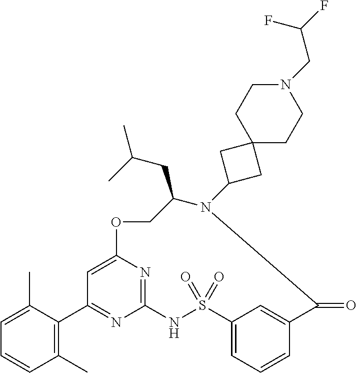 [SMILES: Cc1cccc(C)c1-c1cc2nc(n1)NS(=O)(=O)c1cccc(c1)C(=O)N(C1CC3(CCN(CC(F)F)CC3)C1)[C@H](CC(C)C)CO2] | | | +++ | +++ |
| 321 | 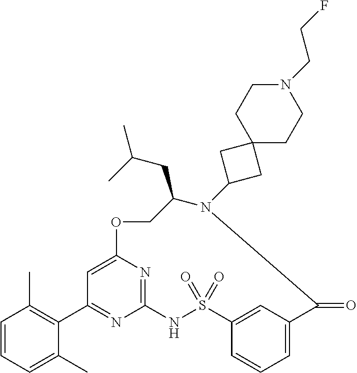 [SMILES: Cc1cccc(C)c1-c1cc2nc(n1)NS(=O)(=O)c1cccc(c1)C(=O)N(C1CC3(CCN(CCF)CC3)C1)[C@H](CC(C)C)CO2] | +++ | +++ | +++ | +++ |
| 20 | 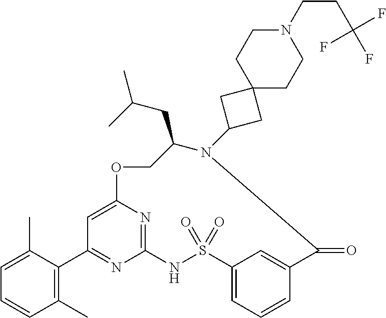 [SMILES: Cc1cccc(C)c1-c1cc2nc(n1)NS(=O)(=O)c1cccc(c1)C(=O)N(C1CC3(CCN(CCC(F)(F)F)CC3)C1)[C@H](CC(C)C)CO2] | +++ | +++ | +++ | +++ |
| 320 | 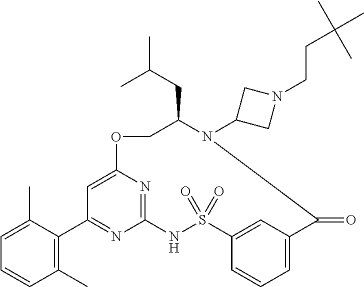 [SMILES: Cc1cccc(C)c1-c1cc2nc(n1)NS(=O)(=O)c1cccc(c1)C(=O)N(C1CN(CCC(C)(C)C)C1)[C@H](CC(C)C)CO2] | | | | |
| 319 | 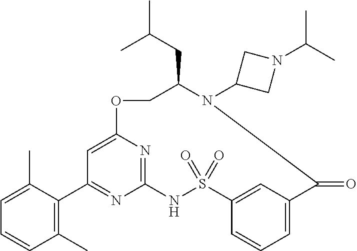 [SMILES: Cc1cccc(C)c1-c1cc2nc(n1)NS(=O)(=O)c1cccc(c1)C(=O)N(C1CN(C(C)C)C1)[C@H](CC(C)C)CO2] | +++ | +++ | +++ | +++ |
| 101 | 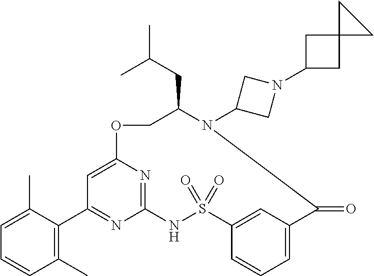 [SMILES: Cc1cccc(C)c1-c1cc2nc(n1)NS(=O)(=O)c1cccc(c1)C(=O)N(C1CN(C3CC4(CC4)C3)C1)[C@H](CC(C)C)CO2] | +++ | +++ | +++ | +++ |
| 318 | 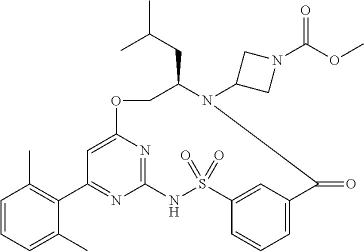 [SMILES: COC(=O)N1CC(N2C(=O)c3cccc(c3)S(=O)(=O)Nc3nc(cc(-c4c(C)cccc4C)n3)OC[C@H]2CC(C)C)C1] | | | | |
| 36 | 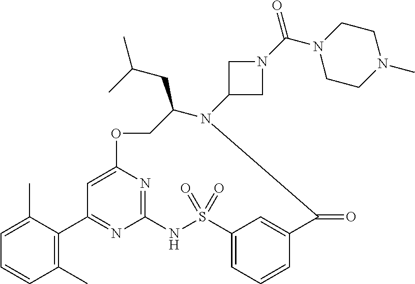 [SMILES: Cc1cccc(C)c1-c1cc2nc(n1)NS(=O)(=O)c1cccc(c1)C(=O)N(C1CN(C(=O)N3CCN(C)CC3)C1)[C@H](CC(C)C)CO2] | +++ | +++ | | |
| 317 | 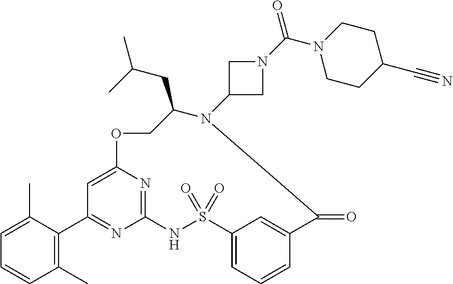 [SMILES: Cc1cccc(C)c1-c1cc2nc(n1)NS(=O)(=O)c1cccc(c1)C(=O)N(C1CN(C(=O)N3CCC(C#N)CC3)C1)[C@H](CC(C)C)CO2] | +++ | +++ | | |
| 316 | 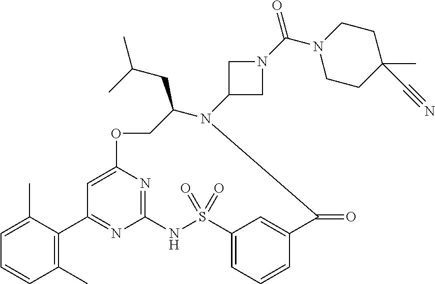 [SMILES: Cc1cccc(C)c1-c1cc2nc(n1)NS(=O)(=O)c1cccc(c1)C(=O)N(C1CN(C(=O)N3CCC(C)(C#N)CC3)C1)[C@H](CC(C)C)CO2] | +++ | +++ | | |
| 21 | 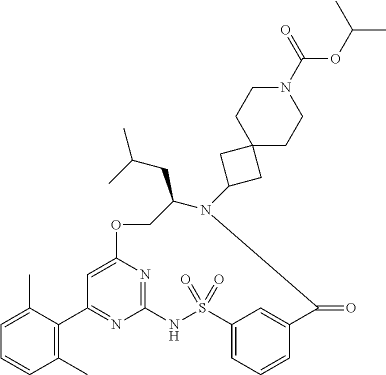 [SMILES: Cc1cccc(C)c1-c1cc2nc(n1)NS(=O)(=O)c1cccc(c1)C(=O)N(C1CC3(CCN(C(=O)OC(C)C)CC3)C1)[C@H](CC(C)C)CO2] | +++ | +++ | +++ | +++ |
| 315 | 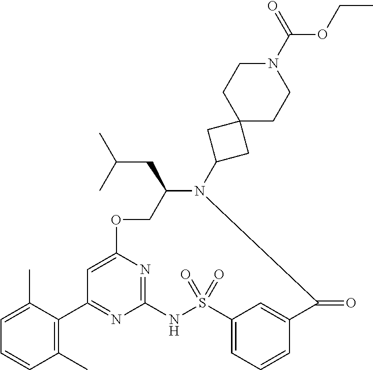 [SMILES: CCOC(=O)N1CCC2(CC1)CC(N1C(=O)c3cccc(c3)S(=O)(=O)Nc3nc(cc(-c4c(C)cccc4C)n3)OC[C@H]1CC(C)C)C2] | +++ | +++ | +++ | +++ |
| 401 | 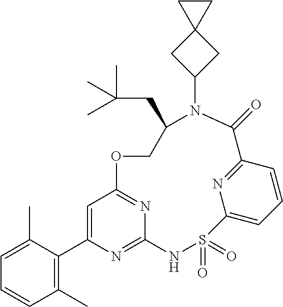 [SMILES: Cc1cccc(C)c1-c1cc2nc(n1)NS(=O)(=O)c1cccc(n1)C(=O)N(C1CC3(CC3)C1)[C@H](CC(C)(C)C)CO2] | +++ | +++ | +++ | +++ |
| 182 | 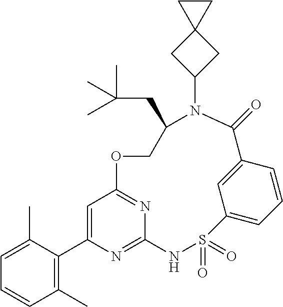 [SMILES: Cc1cccc(C)c1-c1cc2nc(n1)NS(=O)(=O)c1cccc(c1)C(=O)N(C1CC3(CC3)C1)[C@H](CC(C)(C)C)CO2] | +++ | +++ | +++ | +++ |
| 163 | 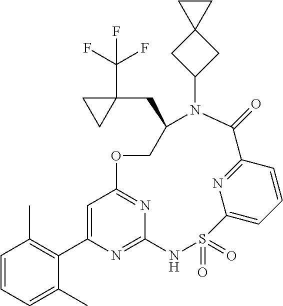 [SMILES: Cc1cccc(C)c1-c1cc2nc(n1)NS(=O)(=O)c1cccc(n1)C(=O)N(C1CC3(CC3)C1)[C@H](CC1(C(F)(F)F)CC1)CO2] | +++ | +++ | | |
| 33 | 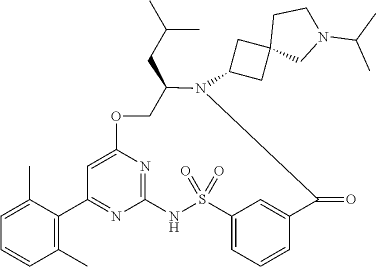 [SMILES: Cc1cccc(C)c1-c1cc2nc(n1)NS(=O)(=O)c1cccc(c1)C(=O)N([C@H]1C[C@]3(CCN(C(C)C)C3)C1)[C@H](CC(C)C)CO2] | +++ | +++ | | |
| 34 | 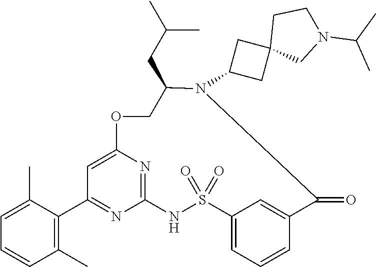 [SMILES: Cc1cccc(C)c1-c1cc2nc(n1)NS(=O)(=O)c1cccc(c1)C(=O)N([C@H]1C[C@]3(CCN(C(C)C)C3)C1)[C@H](CC(C)C)CO2] | +++ | +++ | | |
| 313 | 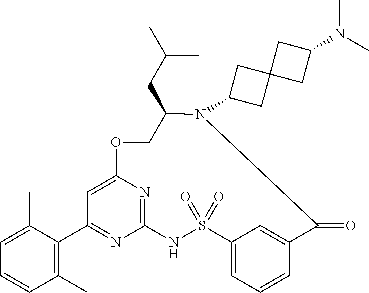 [SMILES: Cc1cccc(C)c1-c1cc2nc(n1)NS(=O)(=O)c1cccc(c1)C(=O)N([C@H]1CC3(C[C@H](N(C)C)C3)C1)[C@H](CC(C)C)CO2] | +++ | +++ | +++ | +++ |
| 314 | 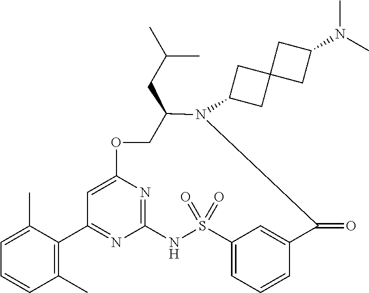 [SMILES: Cc1cccc(C)c1-c1cc2nc(n1)NS(=O)(=O)c1cccc(c1)C(=O)N([C@H]1CC3(C[C@H](N(C)C)C3)C1)[C@H](CC(C)C)CO2] | +++ | +++ | +++ | +++ |
| 22 | 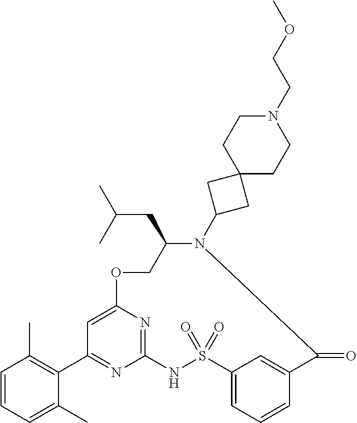 [SMILES: COCCN1CCC2(CC1)CC(N1C(=O)c3cccc(c3)S(=O)(=O)Nc3nc(cc(-c4c(C)cccc4C)n3)OC[C@H]1CC(C)C)C2] | +++ | +++ | +++ | +++ |
| 35 | 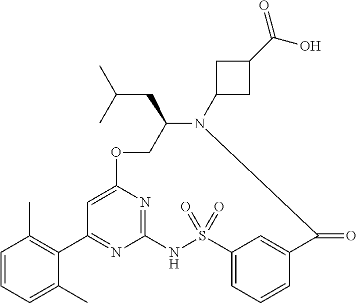 [SMILES: Cc1cccc(C)c1-c1cc2nc(n1)NS(=O)(=O)c1cccc(c1)C(=O)N(C1CC(C(=O)O)C1)[C@H](CC(C)C)CO2] | + | +++ | | |
| 469 | 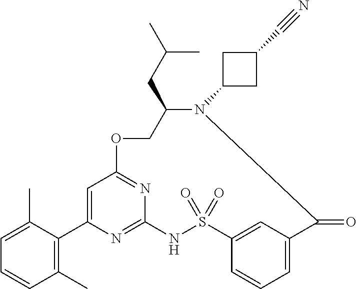 [SMILES: Cc1cccc(C)c1-c1cc2nc(n1)NS(=O)(=O)c1cccc(c1)C(=O)N([C@H]1C[C@@H](C#N)C1)[C@H](CC(C)C)CO2] | +++ | +++ | +++ | +++ |
| 470 | 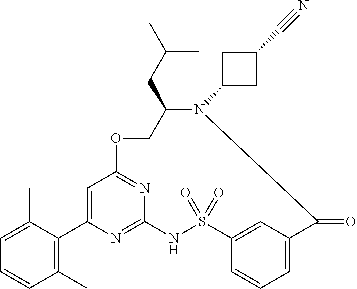 [SMILES: Cc1cccc(C)c1-c1cc2nc(n1)NS(=O)(=O)c1cccc(c1)C(=O)N([C@H]1C[C@@H](C#N)C1)[C@H](CC(C)C)CO2] | +++ | +++ | +++ | +++ |
| 196 | 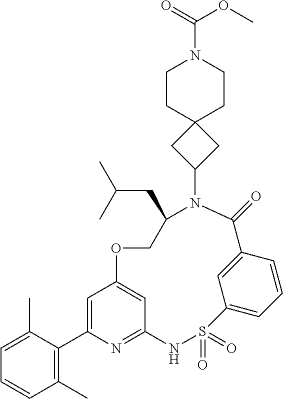 [SMILES: COC(=O)N1CCC2(CC1)CC(N1C(=O)c3cccc(c3)S(=O)(=O)Nc3cc(cc(-c4c(C)cccc4C)n3)OC[C@H]1CC(C)C)C2] | +++ | +++ | | |
| 400 | 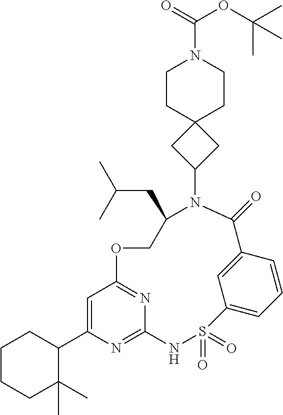 [SMILES: CC(C)C[C@@H]1COc2cc(C3CCCCC3(C)C)nc(n2)NS(=O)(=O)c2cccc(c2)C(=O)N1C1CC2(CCN(C(=O)OC(C)(C)C)CC2)C1] | +++ | ++ | | |
| 399 | 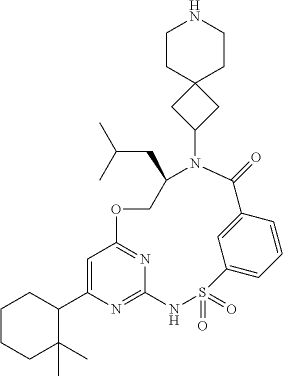 [SMILES: CC(C)C[C@@H]1COc2cc(C3CCCCC3(C)C)nc(n2)NS(=O)(=O)c2cccc(c2)C(=O)N1C1CC2(CCNCC2)C1] | +++ | +++ | | |
| 312 | 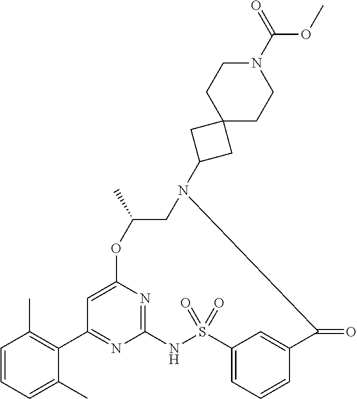 [SMILES: COC(=O)N1CCC2(CC1)CC(N1C[C@@H](C)Oc3cc(-c4c(C)cccc4C)nc(n3)NS(=O)(=O)c3cccc(c3)C1=O)C2] | +++ | +++ | | |
| 179 | 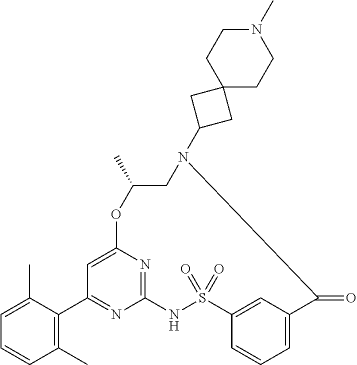 [SMILES: Cc1cccc(C)c1-c1cc2nc(n1)NS(=O)(=O)c1cccc(c1)C(=O)N(C1CC3(CCN(C)CC3)C1)C[C@@H](C)O2] | ++ | +++ | | |
| 311 | 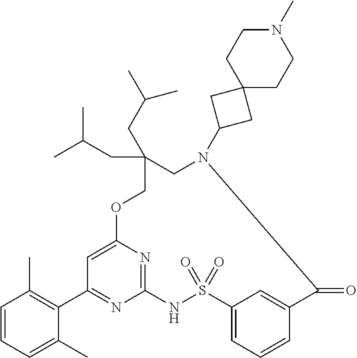 [SMILES: Cc1cccc(C)c1-c1cc2nc(n1)NS(=O)(=O)c1cccc(c1)C(=O)N(C1CC3(CCN(C)CC3)C1)CC(CC(C)C)(CC(C)C)CO2] | +++ | +++ | +++ | +++ |
| 113 | 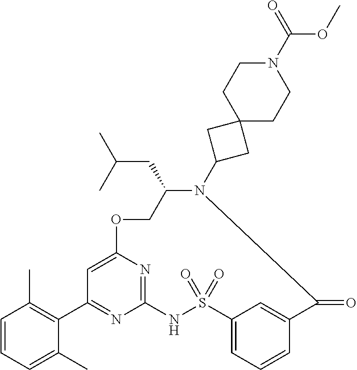 [SMILES: COC(=O)N1CCC2(CC1)CC(N1C(=O)c3cccc(c3)S(=O)(=O)Nc3nc(cc(-c4c(C)cccc4C)n3)OC[C@@H]1CC(C)C)C2] | +++ | ++ | | |
| 395 | 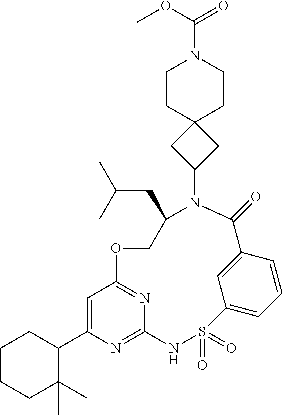 [SMILES: COC(=O)N1CCC2(CC1)CC(N1C(=O)c3cccc(c3)S(=O)(=O)Nc3nc(cc(C4CCCCC4(C)C)n3)OC[C@H]1CC(C)C)C2] | +++ | ++ | | |
| 396 | 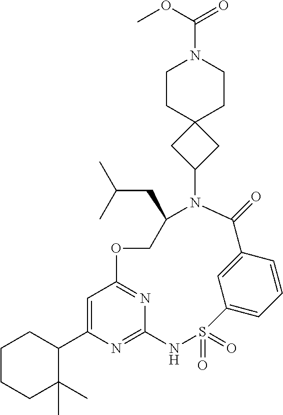 [SMILES: COC(=O)N1CCC2(CC1)CC(N1C(=O)c3cccc(c3)S(=O)(=O)Nc3nc(cc(C4CCCCC4(C)C)n3)OC[C@H]1CC(C)C)C2] | +++ | ++ | | |
| 393 | 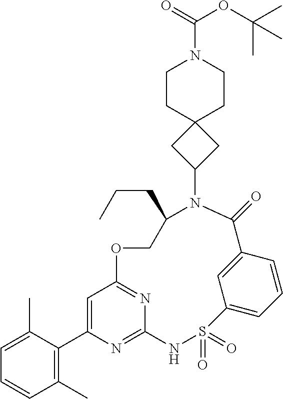 [SMILES: CCC[C@@H]1COc2cc(-c3c(C)cccc3C)nc(n2)NS(=O)(=O)c2cccc(c2)C(=O)N1C1CC2(CCN(C(=O)OC(C)(C)C)CC2)C1] | | | +++ | +++ |
| 392 | 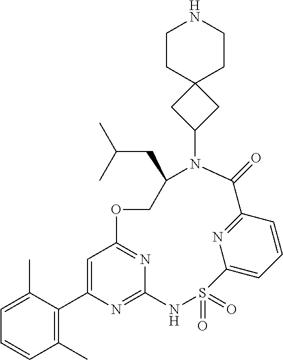 [SMILES: Cc1cccc(C)c1-c1cc2nc(n1)NS(=O)(=O)c1cccc(n1)C(=O)N(C1CC3(CCNCC3)C1)[C@H](CC(C)C)CO2] | | | ++ | +++ |
| 391 | 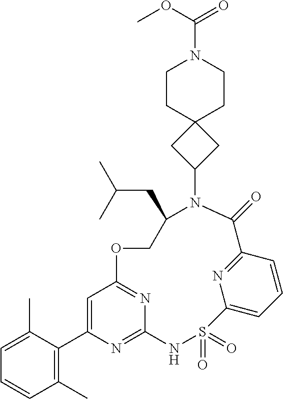 [SMILES: COC(=O)N1CCC2(CC1)CC(N1C(=O)c3cccc(n3)S(=O)(=O)Nc3nc(cc(-c4c(C)cccc4C)n3)OC[C@H]1CC(C)C)C2] | | | +++ | +++ |
| 390 | 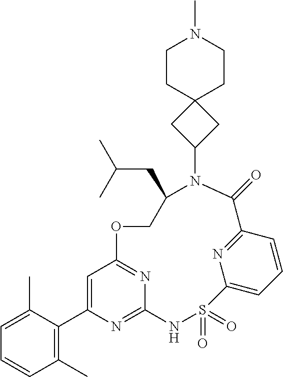 [SMILES: Cc1cccc(C)c1-c1cc2nc(n1)NS(=O)(=O)c1cccc(n1)C(=O)N(C1CC3(CCN(C)CC3)C1)[C@H](CC(C)C)CO2] | | | +++ | +++ |
| 180 | 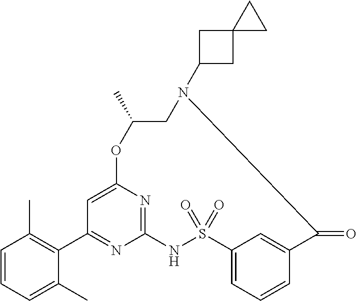 [SMILES: Cc1cccc(C)c1-c1cc2nc(n1)NS(=O)(=O)c1cccc(c1)C(=O)N(C1CC3(CC3)C1)C[C@@H](C)O2] | | | +++ | +++ |
| 181 | 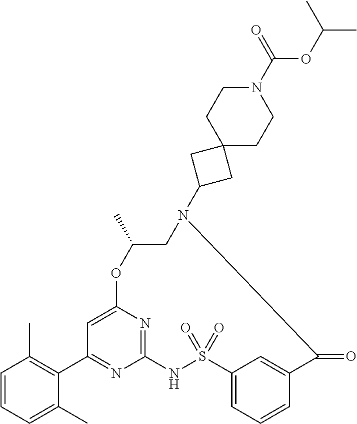 [SMILES: Cc1cccc(C)c1-c1cc2nc(n1)NS(=O)(=O)c1cccc(c1)C(=O)N(C1CC3(CCN(C(=O)OC(C)C)CC3)C1)C[C@@H](C)O2] | | | +++ | +++ |
| 310 | 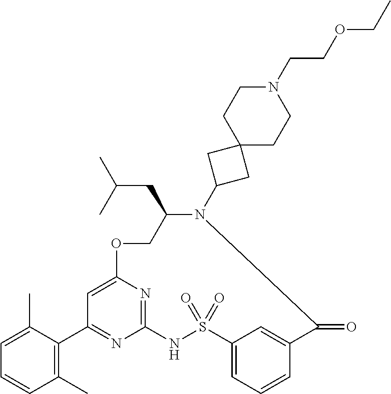 [SMILES: CCOCCN1CCC2(CC1)CC(N1C(=O)c3cccc(c3)S(=O)(=O)Nc3nc(cc(-c4c(C)cccc4C)n3)OC[C@H]1CC(C)C)C2] | | | +++ | +++ |
| 23 | 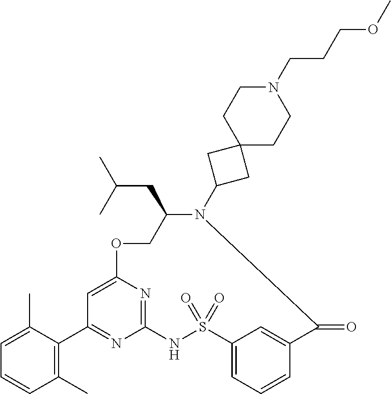 [SMILES: COCCCN1CCC2(CC1)CC(N1C(=O)c3cccc(c3)S(=O)(=O)Nc3nc(cc(-c4c(C)cccc4C)n3)OC[C@H]1CC(C)C)C2] | | | | |
| 468 | 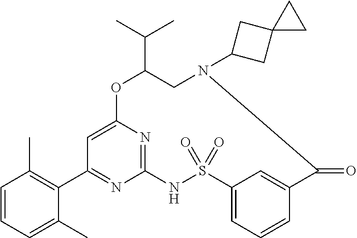 [SMILES: Cc1cccc(C)c1-c1cc2nc(n1)NS(=O)(=O)c1cccc(c1)C(=O)N(C1CC3(CC3)C1)CC(C(C)C)O2] | | | +++ | ++ |
| 394 | 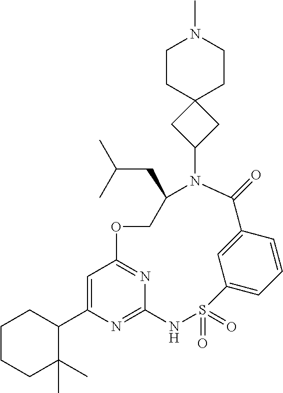 [SMILES: CC(C)C[C@@H]1COc2cc(C3CCCCC3(C)C)nc(n2)NS(=O)(=O)c2cccc(c2)C(=O)N1C1CC2(CCN(C)CC2)C1] | | | ++ | +++ |
| 467 | 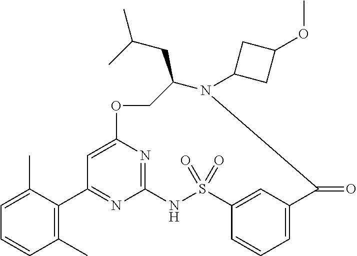 [SMILES: COC1CC(N2C(=O)c3cccc(c3)S(=O)(=O)Nc3nc(cc(-c4c(C)cccc4C)n3)OC[C@H]2CC(C)C)C1] | | | +++ | +++ |
| 309 | 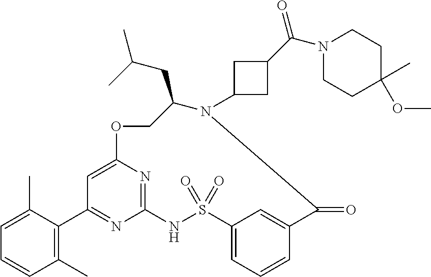 [SMILES: COC1(C)CCN(C(=O)C2CC(N3C(=O)c4cccc(c4)S(=O)(=O)Nc4nc(cc(-c5c(C)cccc5C)n4)OC[C@H]3CC(C)C)C2)CC1] | | | +++ | +++ |
| 308 | 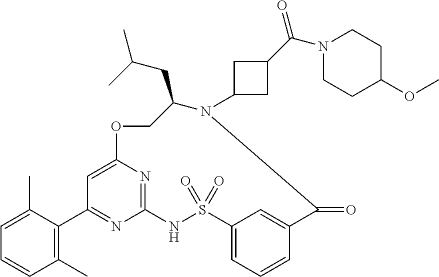 [SMILES: COC1CCN(C(=O)C2CC(N3C(=O)c4cccc(c4)S(=O)(=O)Nc4nc(cc(-c5c(C)cccc5C)n4)OC[C@H]3CC(C)C)C2)CC1] | | | +++ | +++ |
| 307 | 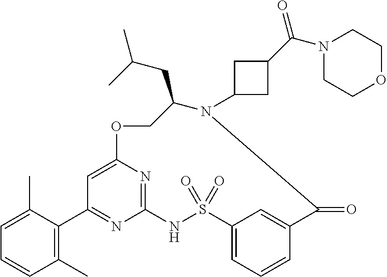 [SMILES: Cc1cccc(C)c1-c1cc2nc(n1)NS(=O)(=O)c1cccc(c1)C(=O)N(C1CC(C(=O)N3CCOCC3)C1)[C@H](CC(C)C)CO2] | | | +++ | +++ |
| 306 | 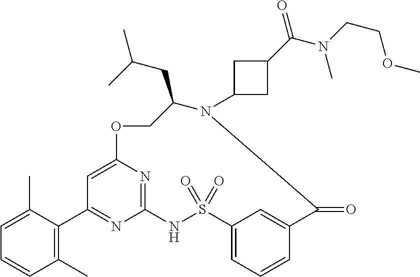 [SMILES: COCCN(C)C(=O)C1CC(N2C(=O)c3cccc(c3)S(=O)(=O)Nc3nc(cc(-c4c(C)cccc4C)n3)OC[C@H]2CC(C)C)C1] | | | +++ | +++ |
| 138 | 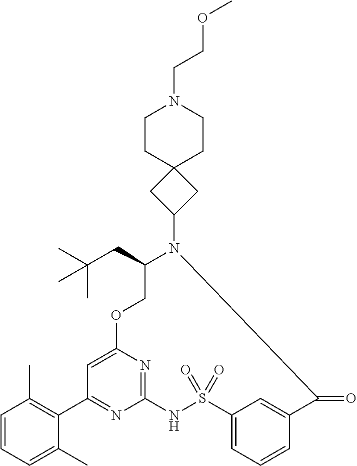 [SMILES: COCCN1CCC2(CC1)CC(N1C(=O)c3cccc(c3)S(=O)(=O)Nc3nc(cc(-c4c(C)cccc4C)n3)OC[C@H]1CC(C)(C)C)C2] | | | +++ | +++ |
| 128 | 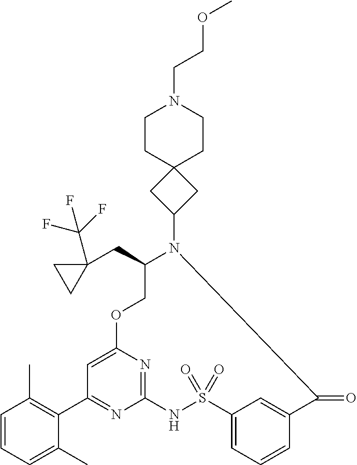 [SMILES: COCCN1CCC2(CC1)CC(N1C(=O)c3cccc(c3)S(=O)(=O)Nc3nc(cc(-c4c(C)cccc4C)n3)OC[C@H]1CC1(C(F)(F)F)CC1)C2] | | | +++ | +++ |
| 442 | 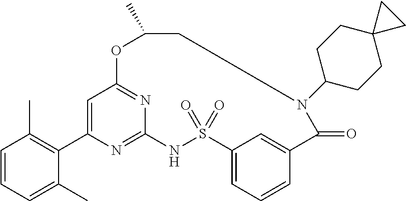 [SMILES: Cc1cccc(C)c1-c1cc2nc(n1)NS(=O)(=O)c1cccc(c1)C(=O)N(C1CCC3(CC1)CC3)C[C@@H](C)O2] | | | +++ | ++ |
| 305 | 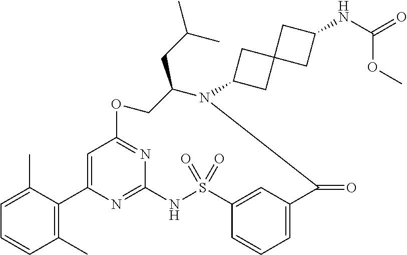 [SMILES: COC(=O)N[C@H]1CC2(C1)C[C@H](N1C(=O)c3cccc(c3)S(=O)(=O)Nc3nc(cc(-c4c(C)cccc4C)n3)OC[C@H]1CC(C)C)C2] | | | +++ | +++ |
| 397 | 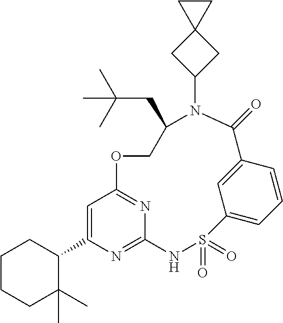 [SMILES: CC(C)(C)C[C@@H]1COc2cc([C@H]3CCCCC3(C)C)nc(n2)NS(=O)(=O)c2cccc(c2)C(=O)N1C1CC2(CC2)C1] | | | +++ | ++ |
| 398 | 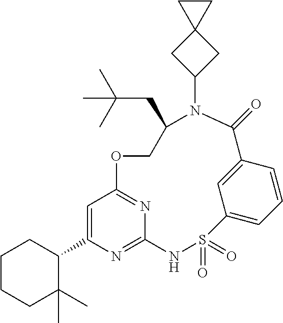 [SMILES: CC(C)(C)C[C@@H]1COc2cc([C@H]3CCCCC3(C)C)nc(n2)NS(=O)(=O)c2cccc(c2)C(=O)N1C1CC2(CC2)C1] | | | +++ | ++ |
| 304 | 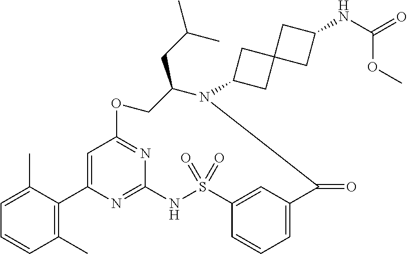 [SMILES: COC(=O)N[C@H]1CC2(C1)C[C@H](N1C(=O)c3cccc(c3)S(=O)(=O)Nc3nc(cc(-c4c(C)cccc4C)n3)OC[C@H]1CC(C)C)C2] | | | +++ | +++ |
| 303 | 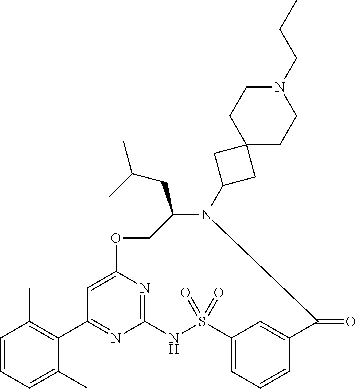 [SMILES: CCCN1CCC2(CC1)CC(N1C(=O)c3cccc(c3)S(=O)(=O)Nc3nc(cc(-c4c(C)cccc4C)n3)OC[C@H]1CC(C)C)C2] | | | +++ | +++ |
| 164 | 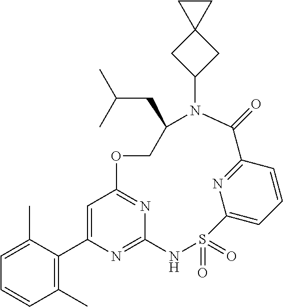 [SMILES: Cc1cccc(C)c1-c1cc2nc(n1)NS(=O)(=O)c1cccc(n1)C(=O)N(C1CC3(CC3)C1)[C@H](CC(C)C)CO2] | | | +++ | +++ |
| 389 | 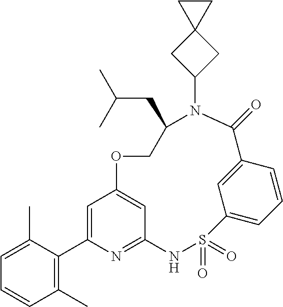 [SMILES: Cc1cccc(C)c1-c1cc2cc(n1)NS(=O)(=O)c1cccc(c1)C(=O)N(C1CC3(CC3)C1)[C@H](CC(C)C)CO2] | | | +++ | +++ |
| 302 | 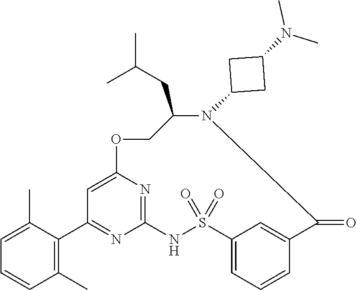 [SMILES: Cc1cccc(C)c1-c1cc2nc(n1)NS(=O)(=O)c1cccc(c1)C(=O)N([C@H]1C[C@@H](N(C)C)C1)[C@H](CC(C)C)CO2] | | | +++ | +++ |
| 301 | 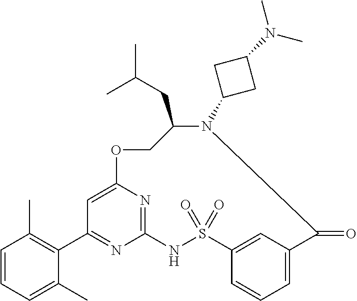 [SMILES: Cc1cccc(C)c1-c1cc2nc(n1)NS(=O)(=O)c1cccc(c1)C(=O)N([C@H]1C[C@@H](N(C)C)C1)[C@H](CC(C)C)CO2] | | | +++ | +++ |
| 63 | 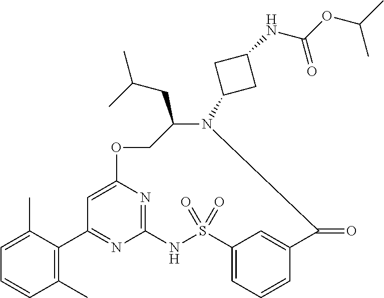 [SMILES: Cc1cccc(C)c1-c1cc2nc(n1)NS(=O)(=O)c1cccc(c1)C(=O)N([C@H]1C[C@@H](NC(=O)OC(C)C)C1)[C@H](CC(C)C)CO2] | | | +++ | +++ |
| 64 | 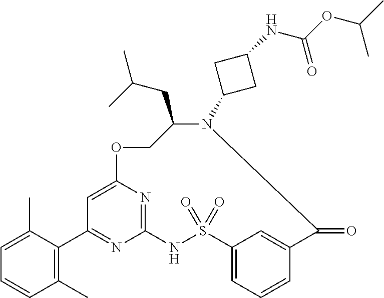 [SMILES: Cc1cccc(C)c1-c1cc2nc(n1)NS(=O)(=O)c1cccc(c1)C(=O)N([C@H]1C[C@@H](NC(=O)OC(C)C)C1)[C@H](CC(C)C)CO2] | | | +++ | +++ |
| 197 | 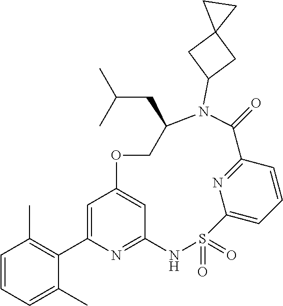 [SMILES: Cc1cccc(C)c1-c1cc2cc(n1)NS(=O)(=O)c1cccc(n1)C(=O)N(C1CC3(CC3)C1)[C@H](CC(C)C)CO2] | | | +++ | +++ |
| 127 | 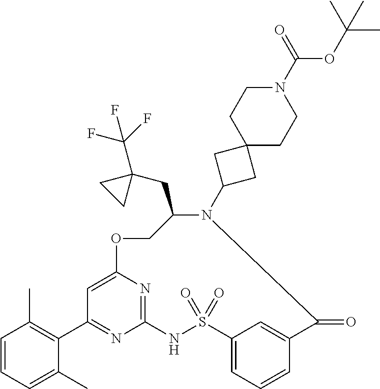 [SMILES: Cc1cccc(C)c1-c1cc2nc(n1)NS(=O)(=O)c1cccc(c1)C(=O)N(C1CC3(CCN(C(=O)OC(C)(C)C)CC3)C1)[C@H](CC1(C(F)(F)F)CC1)CO2] | | | +++ | +++ |
| 464 | 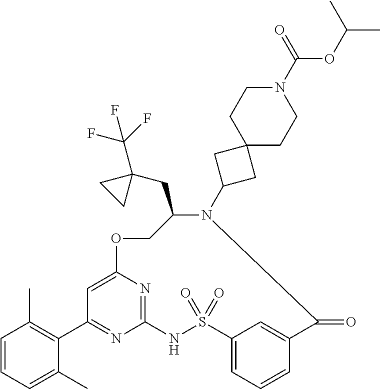 [SMILES: Cc1cccc(C)c1-c1cc2nc(n1)NS(=O)(=O)c1cccc(c1)C(=O)N(C1CC3(CCN(C(=O)OC(C)C)CC3)C1)[C@H](CC1(C(F)(F)F)CC1)CO2] | | | +++ | +++ |
| 102 | 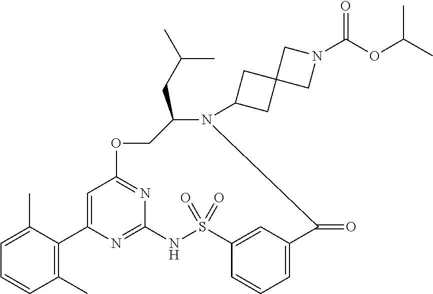 [SMILES: Cc1cccc(C)c1-c1cc2nc(n1)NS(=O)(=O)c1cccc(c1)C(=O)N(C1CC3(C1)CN(C(=O)OC(C)C)C3)[C@H](CC(C)C)CO2] | | | +++ | +++ |
| 103 | 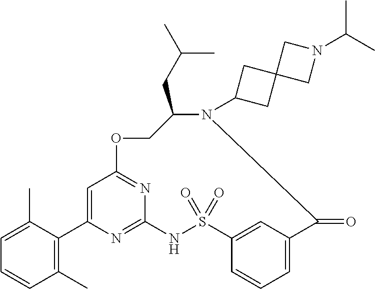 [SMILES: Cc1cccc(C)c1-c1cc2nc(n1)NS(=O)(=O)c1cccc(c1)C(=O)N(C1CC3(C1)CN(C(C)C)C3)[C@H](CC(C)C)CO2] | | | +++ | +++ |
| 300 | 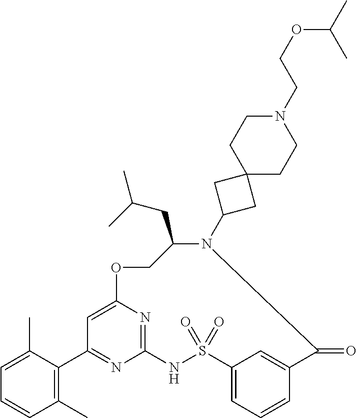 [SMILES: Cc1cccc(C)c1-c1cc2nc(n1)NS(=O)(=O)c1cccc(c1)C(=O)N(C1CC3(CCN(CCOC(C)C)CC3)C1)[C@H](CC(C)C)CO2] | | | +++ | ++++ |
| 129 | 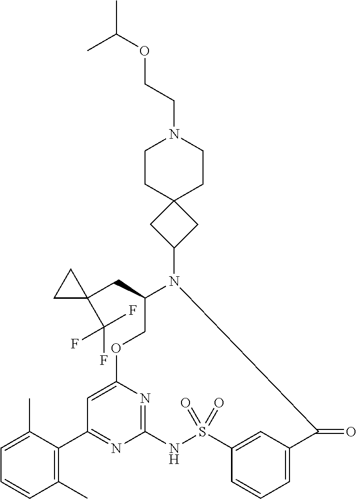 [SMILES: Cc1cccc(C)c1-c1cc2nc(n1)NS(=O)(=O)c1cccc(c1)C(=O)N(C1CC3(CCN(CCOC(C)C)CC3)C1)[C@H](CC1(C(F)(F)F)CC1)CO2] | | | +++ | +++ |
| 215 | 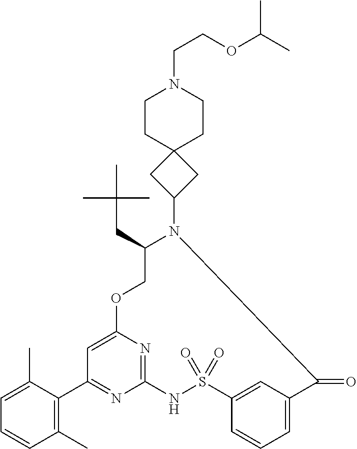 [SMILES: Cc1cccc(C)c1-c1cc2nc(n1)NS(=O)(=O)c1cccc(c1)C(=O)N(C1CC3(CCN(CCOC(C)C)CC3)C1)[C@H](CC(C)(C)C)CO2] | | | +++ | +++ |
| 134 | 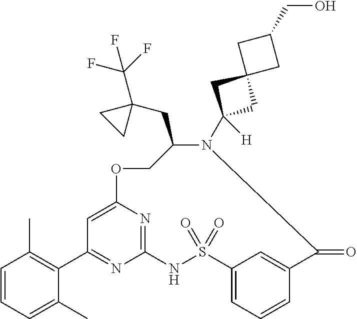 | | | +++ | +++ |
| 135 | 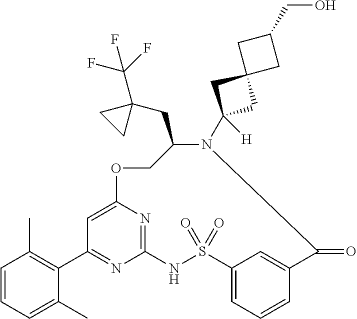 | | | +++ | +++ |
| 465 | 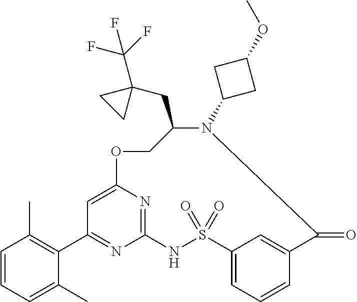 [SMILES: CO[C@H]1C[C@@H](N2C(=O)c3cccc(c3)S(=O)(=O)Nc3nc(cc(-c4c(C)cccc4C)n3)OC[C@H]2CC2(C(F)(F)F)CC2)C1] | | | +++ | +++ |
| 466 | 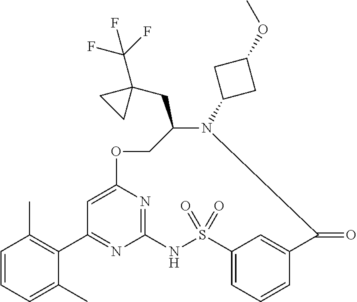 [SMILES: CO[C@H]1C[C@@H](N2C(=O)c3cccc(c3)S(=O)(=O)Nc3nc(cc(-c4c(C)cccc4C)n3)OC[C@H]2CC2(C(F)(F)F)CC2)C1] | | | +++ | +++ |
| 298 | 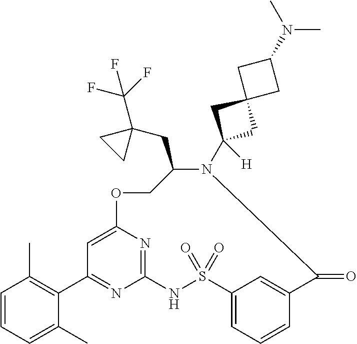 | | | +++ | +++ |
| 297 | 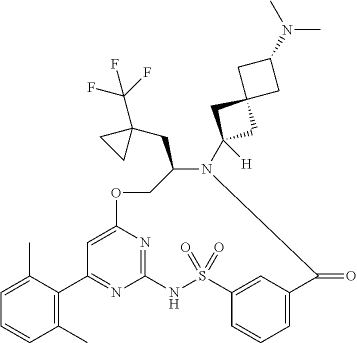 | | | +++ | +++ |
| 62 | 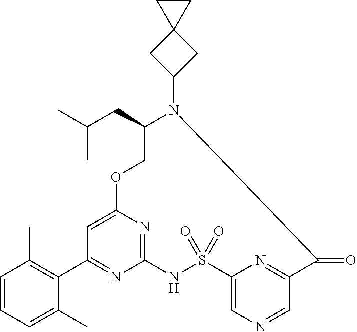 [SMILES: Cc1cccc(C)c1-c1cc2nc(n1)NS(=O)(=O)c1cncc(n1)C(=O)N(C1CC3(CC3)C1)[C@H](CC(C)C)CO2] | | | +++ | +++ |
| 78 | 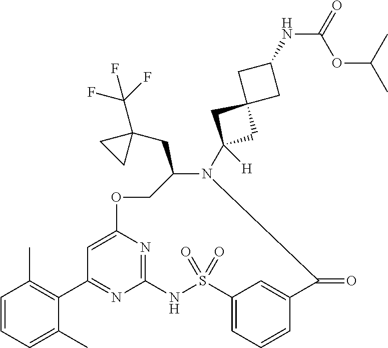 | | | +++ | +++ |
| 79 | 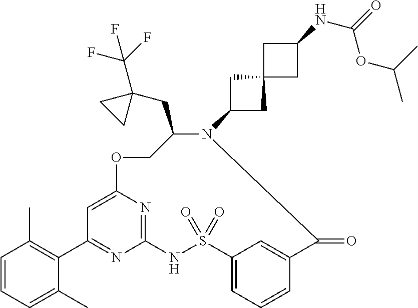 | | | +++ | +++ |
| 299 | 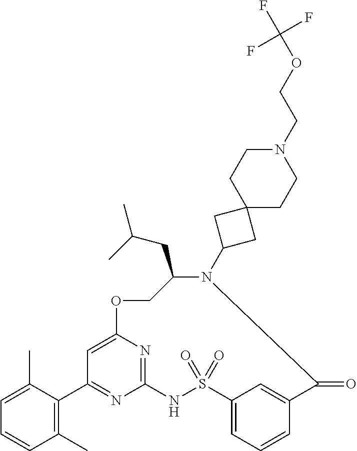 [SMILES: Cc1cccc(C)c1-c1cc2nc(n1)NS(=O)(=O)c1cccc(c1)C(=O)N(C1CC3(CCN(CCOC(F)(F)F)CC3)C1)[C@H](CC(C)C)CO2] | | | +++ | +++ |
| 296 | 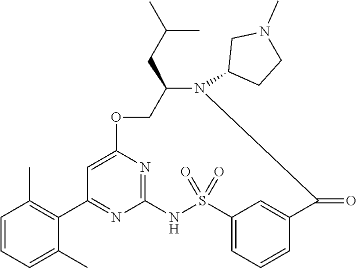 [SMILES: Cc1cccc(C)c1-c1cc2nc(n1)NS(=O)(=O)c1cccc(c1)C(=O)N([C@H]1CCN(C)C1)[C@H](CC(C)C)CO2] | | | + | +++ |
| 295 | 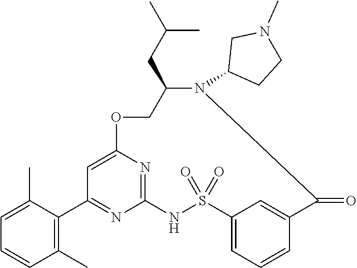 [SMILES: Cc1cccc(C)c1-c1cc2nc(n1)NS(=O)(=O)c1cccc(c1)C(=O)N([C@H]1CCN(C)C1)[C@H](CC(C)C)CO2] | | | +++ | +++ |
| 294 | 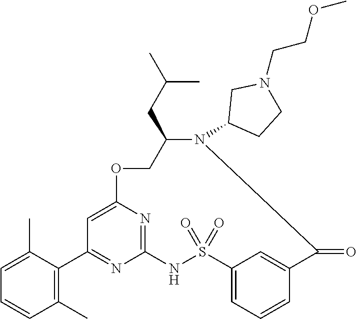 [SMILES: COCCN1CC[C@H](N2C(=O)c3cccc(c3)S(=O)(=O)Nc3nc(cc(-c4c(C)cccc4C)n3)OC[C@H]2CC(C)C)C1] | | | +++ | +++ |
| 38 | 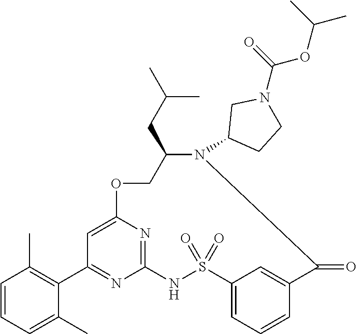 [SMILES: Cc1cccc(C)c1-c1cc2nc(n1)NS(=O)(=O)c1cccc(c1)C(=O)N([C@H]1CCN(C(=O)OC(C)C)C1)[C@H](CC(C)C)CO2] | | | +++ | ++ |
| 39 | 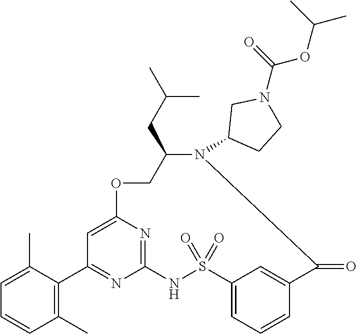 [SMILES: Cc1cccc(C)c1-c1cc2nc(n1)NS(=O)(=O)c1cccc(c1)C(=O)N([C@H]1CCN(C(=O)OC(C)C)C1)[C@H](CC(C)C)CO2] | | | +++ | +++ |
| 437 | 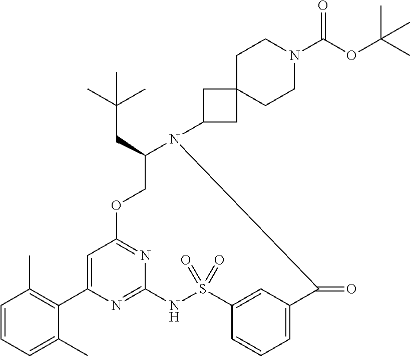 [SMILES: Cc1cccc(C)c1-c1cc2nc(n1)NS(=O)(=O)c1cccc(c1)C(=O)N(C1CC3(CCN(C(=O)OC(C)(C)C)CC3)C1)[C@H](CC(C)(C)C)CO2] | | | +++ | ++ |
| 150 | 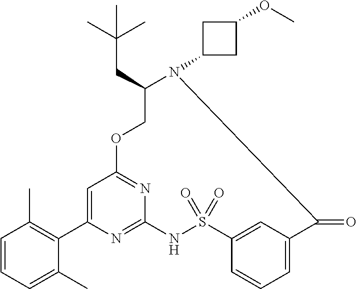 [SMILES: CO[C@H]1C[C@@H](N2C(=O)c3cccc(c3)S(=O)(=O)Nc3nc(cc(-c4c(C)cccc4C)n3)OC[C@H]2CC(C)(C)C)C1] | | | +++ | +++ |
| 151 | 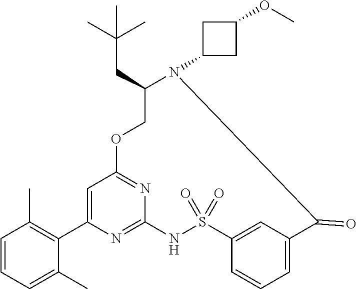 [SMILES: CO[C@H]1C[C@@H](N2C(=O)c3cccc(c3)S(=O)(=O)Nc3nc(cc(-c4c(C)cccc4C)n3)OC[C@H]2CC(C)(C)C)C1] | | | +++ | +++ |
| 118 | 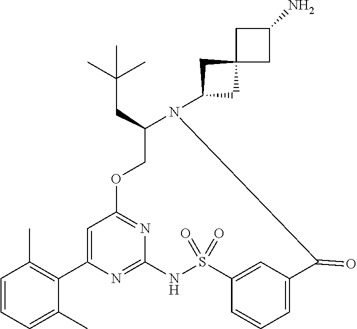 | | | +++ | +++ |

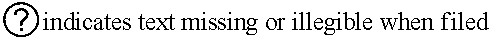

Table 9 represents CFTR modulating activity for representative compounds of the disclosure generated using one or more of the assays disclosed herein (EC50: +++ is <1 μM; ++ is 1-<3 μM; + is 3-<30 μM; and ND is “not detected in this assay.” % Activity: +++ is >60%; ++ is 30-60%; + is <300%).

TABLE 9
Biological Activity Data

| | | | Ent. A | | Ent. B |
|---|---|---|---|---|---|
| | | Ent. A | Max | Ent. B | Max |
| Cmpd | | EC₅₀ | Activity | EC₅₀ | Activity |
| No. | Structure | (μM) | (%) | (μM) | (%) |
| 463 | 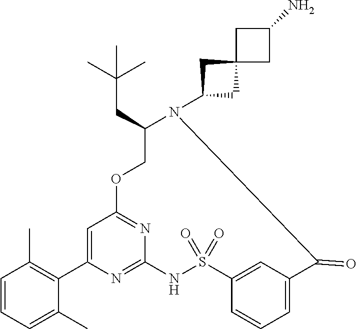 | | | +++ | +++ |
| 47 | 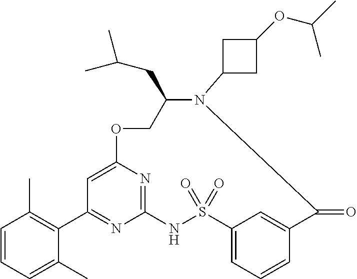 [SMILES: Cc1cccc(C)c1-c1cc2nc(n1)NS(=O)(=O)c1cccc(c1)C(=O)N(C1CC(OC(C)C)C1)[C@H](CC(C)C)CO2] | | | +++ | +++ |
| 88 | 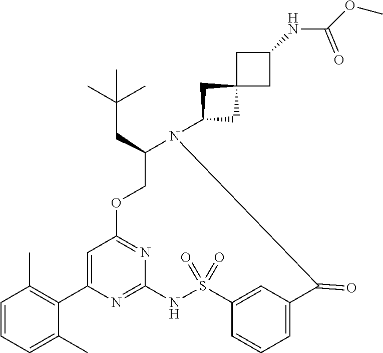 | | | +++ | +++ |
| 91 | 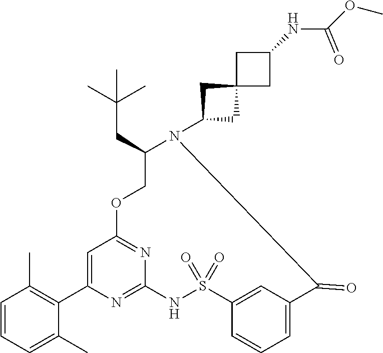 | | | +++ | +++ |
| 92 | 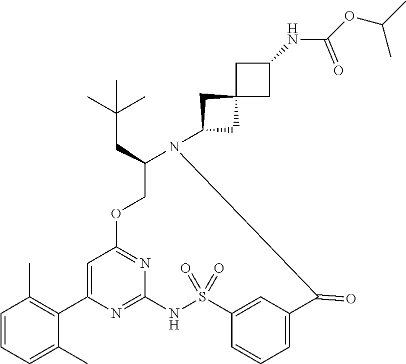 | | | +++ | +++ |
| 93 | 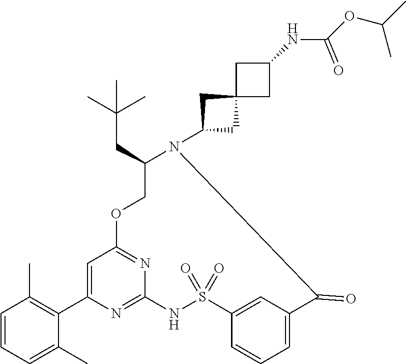 | | | +++ | +++ |
| 462 | 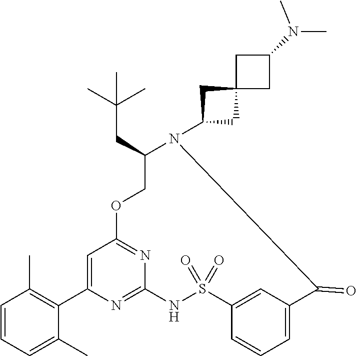 | | | +++ | +++ |
| 461 | 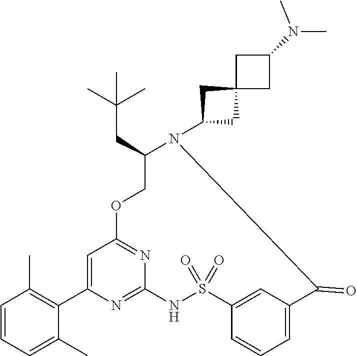 | | | +++ | +++ |
| 425 | 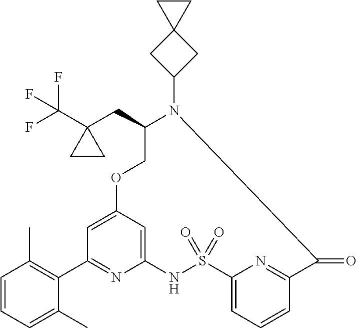 [SMILES: Cc1cccc(C)c1-c1cc2cc(n1)NS(=O)(=O)c1cccc(n1)C(=O)N(C1CC3(CC3)C1)[C@H](CC1(C(F)(F)F)CC1)CO2] | | | +++ | +++ |
| 37 | 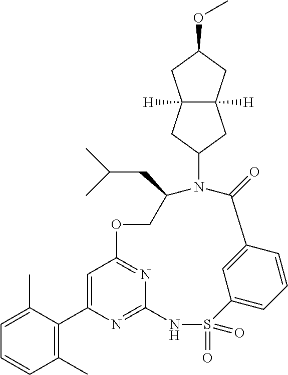 [SMILES: CO[C@H]1C[C@H]2CC(N3C(=O)c4cccc(c4)S(=O)(=O)Nc4nc(cc(-c5c(C)cccc5C)n4)OC[C@H]3CC(C)C)C[C@H]2C1] | | | +++ | +++ |
| 74 | 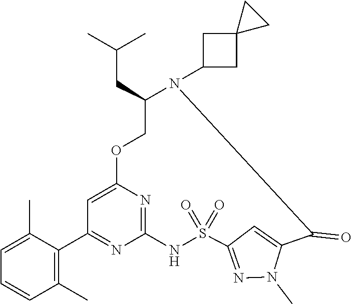 [SMILES: Cc1cccc(C)c1-c1cc2nc(n1)NS(=O)(=O)c1cc(n(C)n1)C(=O)N(C1CC3(CC3)C1)[C@H](CC(C)C)CO2] | | | +++ | +++ |
| 293 | 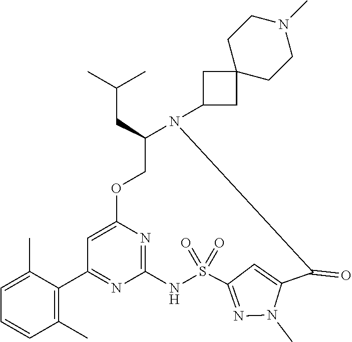 [SMILES: Cc1cccc(C)c1-c1cc2nc(n1)NS(=O)(=O)c1cc(n(C)n1)C(=O)N(C1CC3(CCN(C)CC3)C1)[C@H](CC(C)C)CO2] | | | ++ | +++ |
| 440 | 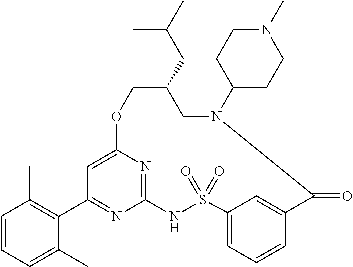 [SMILES: Cc1cccc(C)c1-c1cc2nc(n1)NS(=O)(=O)c1cccc(c1)C(=O)N(C1CCN(C)CC1)C[C@@H](CC(C)C)CO2] | | | ++ | ++ |
| 441 | 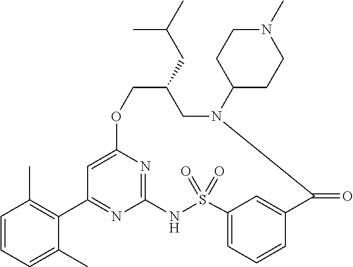 [SMILES: Cc1cccc(C)c1-c1cc2nc(n1)NS(=O)(=O)c1cccc(c1)C(=O)N(C1CCN(C)CC1)C[C@@H](CC(C)C)CO2] | | | +++ | +++ |
| 45 | 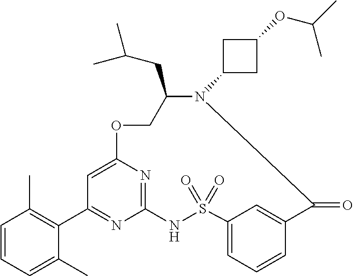 [SMILES: Cc1cccc(C)c1-c1cc2nc(n1)NS(=O)(=O)c1cccc(c1)C(=O)N([C@H]1C[C@@H](OC(C)C)C1)[C@H](CC(C)C)CO2] | | | +++ | +++ |
| 46 | 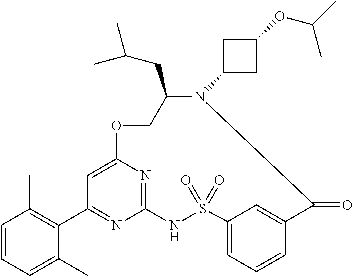 [SMILES: Cc1cccc(C)c1-c1cc2nc(n1)NS(=O)(=O)c1cccc(c1)C(=O)N([C@H]1C[C@@H](OC(C)C)C1)[C@H](CC(C)C)CO2] | | | +++ | +++ |
| 292 | 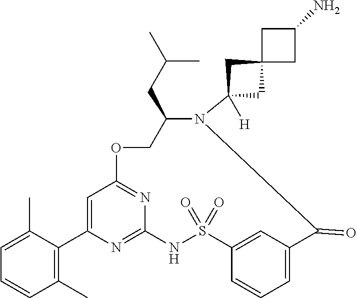 | | | +++ | +++ |
| 291 | 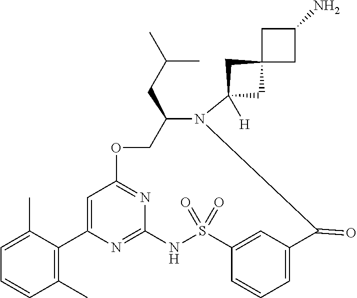 | | | +++ | +++ |
| 130 | 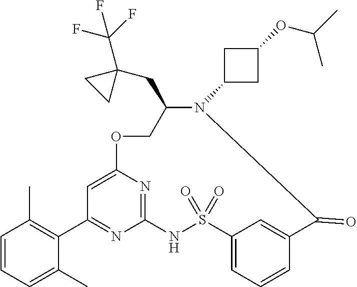 [SMILES: Cc1cccc(C)c1-c1cc2nc(n1)NS(=O)(=O)c1cccc(c1)C(=O)N([C@H]1C[C@@H](OC(C)C)C1)[C@H](CC1(C(F)(F)F)CC1)CO2] | | | +++ | +++ |
| 131 | 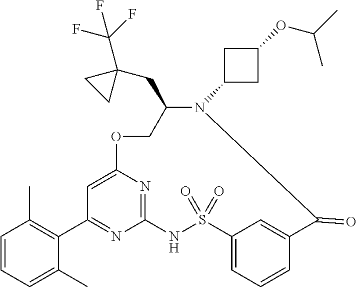 [SMILES: Cc1cccc(C)c1-c1cc2nc(n1)NS(=O)(=O)c1cccc(c1)C(=O)N([C@H]1C[C@@H](OC(C)C)C1)[C@H](CC1(C(F)(F)F)CC1)CO2] | | | +++ | +++ |
| 24 | 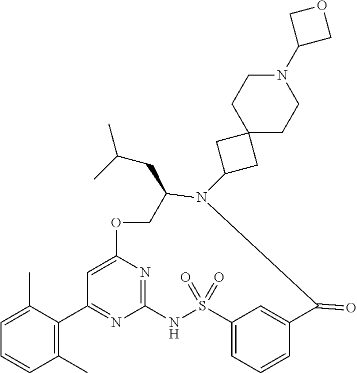 [SMILES: Cc1cccc(C)c1-c1cc2nc(n1)NS(=O)(=O)c1cccc(c1)C(=O)N(C1CC3(CCN(C4COC4)CC3)C1)[C@H](CC(C)C)CO2] | | | +++ | +++ |
| 290 | 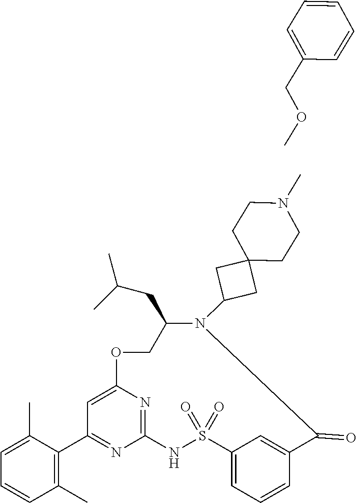 | | | +++ | +++ |
| 289 | 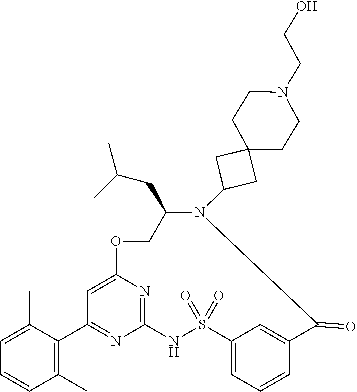 [SMILES: Cc1cccc(C)c1-c1cc2nc(n1)NS(=O)(=O)c1cccc(c1)C(=O)N(C1CC3(CCN(CCO)CC3)C1)[C@H](CC(C)C)CO2] | | | +++ | ++ |
| 282 | 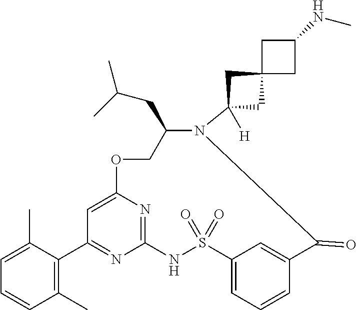 | | | +++ | +++ |
| 281 | 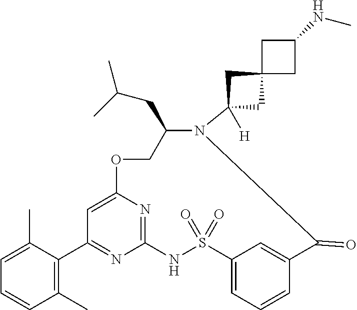 | | | +++ | +++ |
| 288 | 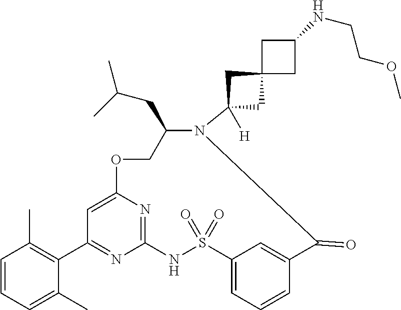 | | | +++ | +++ |
| 287 | 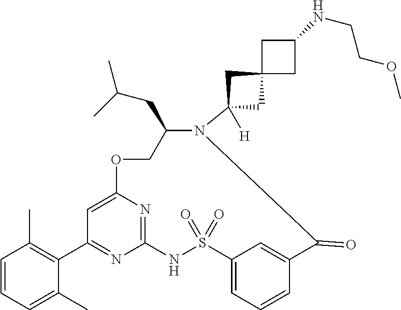 | | | +++ | +++ |
| 286 | 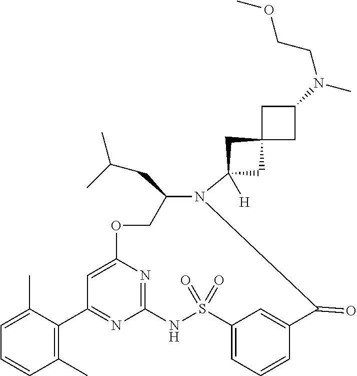 | | | +++ | +++ |
| 285 | 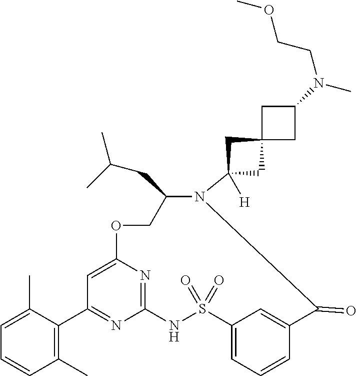 | | | +++ | +++ |
| 284 | 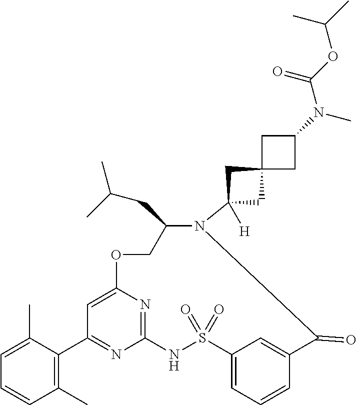 | | | +++ | ++ |
| 283 | 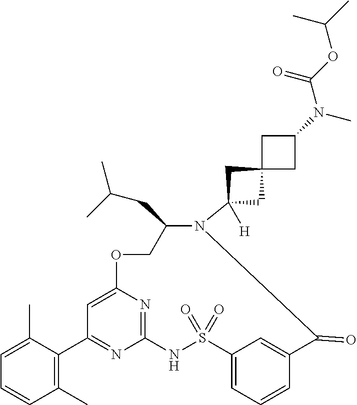 | | | +++ | ++ |
| 460 | 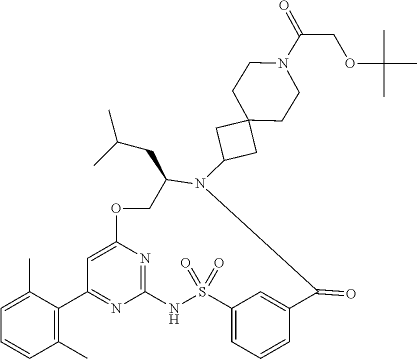 [SMILES: Cc1cccc(C)c1-c1cc2nc(n1)NS(=O)(=O)c1cccc(c1)C(=O)N(C1CC3(CCN(C(=O)COC(C)(C)C)CC3)C1)[C@H](CC(C)C)CO2] | | | +++ | +++ |
| 459 | 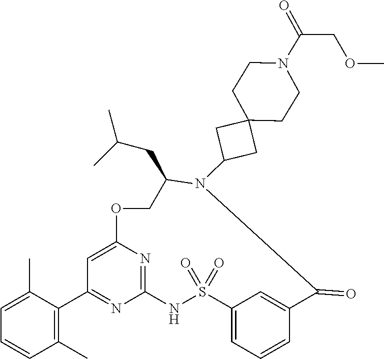 [SMILES: COCC(=O)N1CCC2(CC1)CC(N1C(=O)c3cccc(c3)S(=O)(=O)Nc3nc(cc(-c4c(C)cccc4C)n3)OC[C@H]1CC(C)C)C2] | | | +++ | +++ |
| 458 | 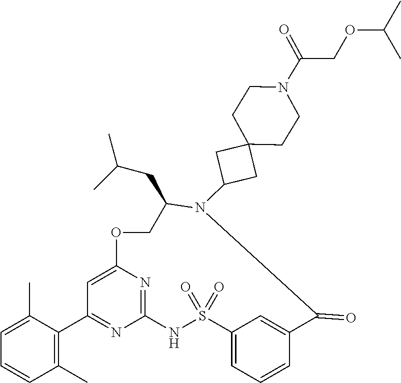 [SMILES: Cc1cccc(C)c1-c1cc2nc(n1)NS(=O)(=O)c1cccc(c1)C(=O)N(C1CC3(CCN(C(=O)COC(C)C)CC3)C1)[C@H](CC(C)C)CO2] | | | +++ | +++ |
| 457 | 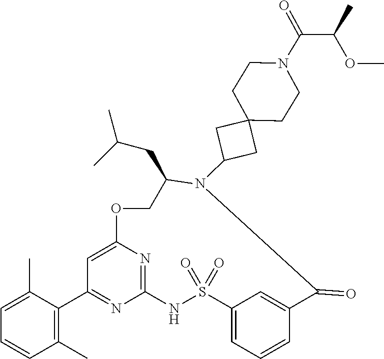 [SMILES: CO[C@H](C)C(=O)N1CCC2(CC1)CC(N1C(=O)c3cccc(c3)S(=O)(=O)Nc3nc(cc(-c4c(C)cccc4C)n3)OC[C@H]1CC(C)C)C2] | | | +++ | +++ |
| 25 | 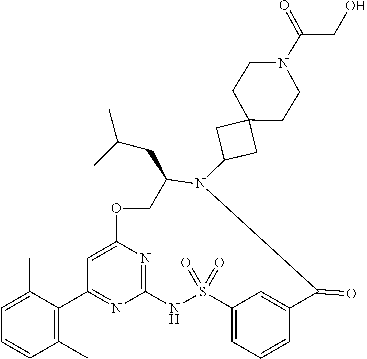 [SMILES: Cc1cccc(C)c1-c1cc2nc(n1)NS(=O)(=O)c1cccc(c1)C(=O)N(C1CC3(CCN(C(=O)CO)CC3)C1)[C@H](CC(C)C)CO2] | | | +++ | +++ |
| 10 | 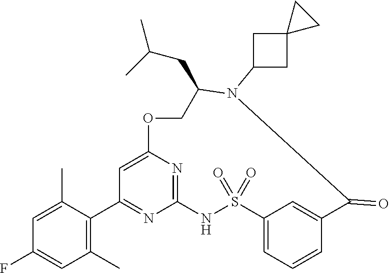 [SMILES: Cc1cc(F)cc(C)c1-c1cc2nc(n1)NS(=O)(=O)c1cccc(c1)C(=O)N(C1CC3(CC3)C1)[C@H](CC(C)C)CO2] | | | +++ | +++ |
| 278 | 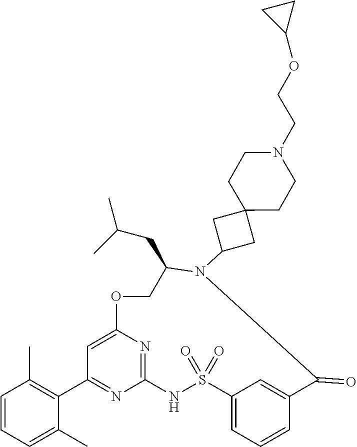 [SMILES: Cc1cccc(C)c1-c1cc2nc(n1)NS(=O)(=O)c1cccc(c1)C(=O)N(C1CC3(CCN(CCOC4CC4)CC3)C1)[C@H](CC(C)C)CO2] | | | +++ | +++ |
| 279 | 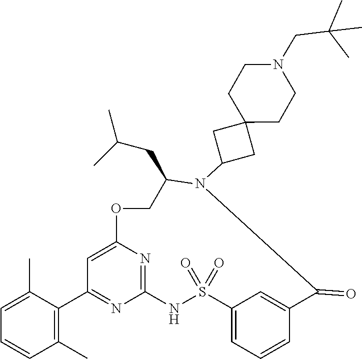 [SMILES: Cc1cccc(C)c1-c1cc2nc(n1)NS(=O)(=O)c1cccc(c1)C(=O)N(C1CC3(CCN(CC(C)(C)C)CC3)C1)[C@H](CC(C)C)CO2] | | | +++ | +++ |
| 280 | 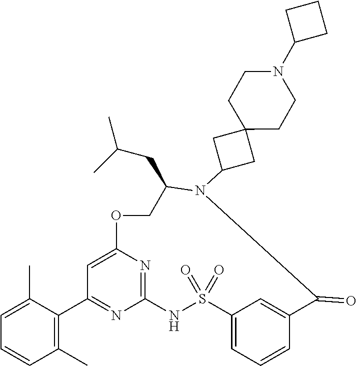 [SMILES: Cc1cccc(C)c1-c1cc2nc(n1)NS(=O)(=O)c1cccc(c1)C(=O)N(C1CC3(CCN(C4CCC4)CC3)C1)[C@H](CC(C)C)CO2] | | | +++ | +++ |
| 388 | 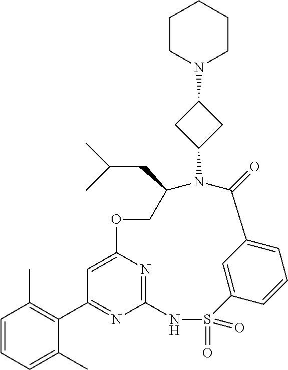 [SMILES: Cc1cccc(C)c1-c1cc2nc(n1)NS(=O)(=O)c1cccc(c1)C(=O)N([C@H]1C[C@@H](N3CCCCC3)C1)[C@H](CC(C)C)CO2] | | | +++ | +++ |
| 387 | 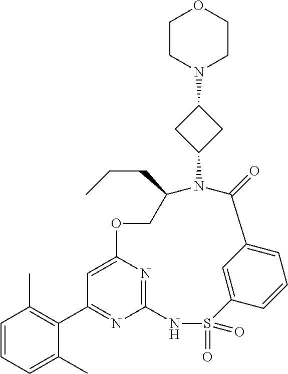 [SMILES: CCC[C@@H]1COc2cc(-c3c(C)cccc3C)nc(n2)NS(=O)(=O)c2cccc(c2)C(=O)N1[C@H]1C[C@@H](N2CCOCC2)C1] | | | +++ | +++ |
| 386 | 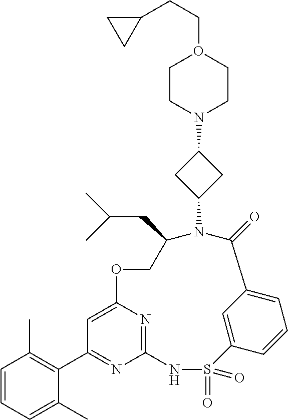 | | | +++ | +++ |
| 456 | 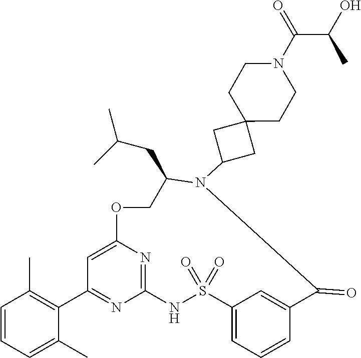 [SMILES: Cc1cccc(C)c1-c1cc2nc(n1)NS(=O)(=O)c1cccc(c1)C(=O)N(C1CC3(CCN(C(=O)[C@H](C)O)CC3)C1)[C@H](CC(C)C)CO2] | | | +++ | +++ |
| 455 | 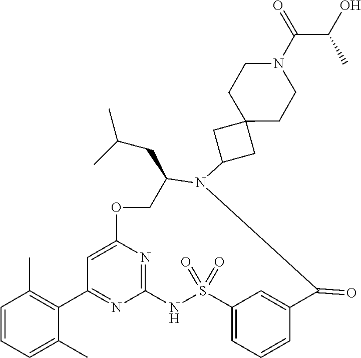 [SMILES: Cc1cccc(C)c1-c1cc2nc(n1)NS(=O)(=O)c1cccc(c1)C(=O)N(C1CC3(CCN(C(=O)[C@@H](C)O)CC3)C1)[C@H](CC(C)C)CO2] | | | +++ | +++ |
| 454 | 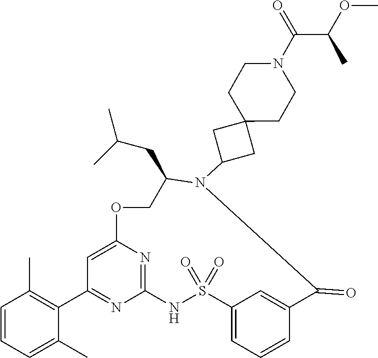 [SMILES: CO[C@@H](C)C(=O)N1CCC2(CC1)CC(N1C(=O)c3cccc(c3)S(=O)(=O)Nc3nc(cc(-c4c(C)cccc4C)n3)OC[C@H]1CC(C)C)C2] | | | +++ | +++ |
| 104 | 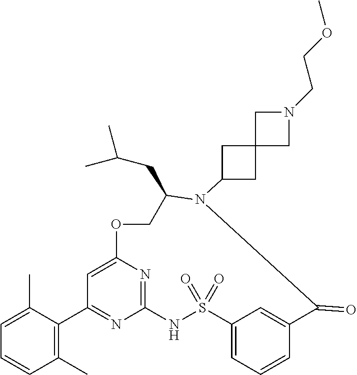 [SMILES: COCCN1CC2(CC(N3C(=O)c4cccc(c4)S(=O)(=O)Nc4nc(cc(-c5c(C)cccc5C)n4)OC[C@H]3CC(C)C)C2)C1] | | | | +++ |
| 276 | 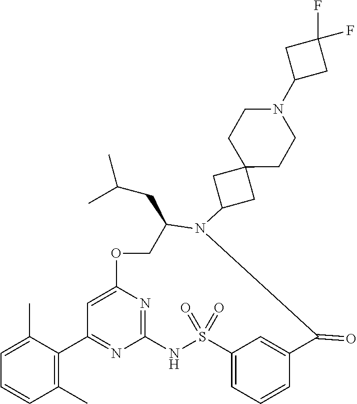 [SMILES: Cc1cccc(C)c1-c1cc2nc(n1)NS(=O)(=O)c1cccc(c1)C(=O)N(C1CC3(CCN(C4CC(F)(F)C4)CC3)C1)[C@H](CC(C)C)CO2] | | | +++ | +++ |
| 275 | 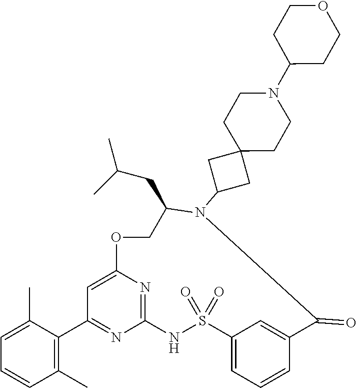 [SMILES: Cc1cccc(C)c1-c1cc2nc(n1)NS(=O)(=O)c1cccc(c1)C(=O)N(C1CC3(CCN(C4CCOCC4)CC3)C1)[C@H](CC(C)C)CO2] | | | +++ | +++ |
| 110 | 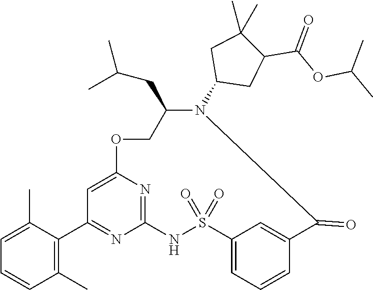 [SMILES: Cc1cccc(C)c1-c1cc2nc(n1)NS(=O)(=O)c1cccc(c1)C(=O)N([C@H]1CC(C(=O)OC(C)C)C(C)(C)C1)[C@H](CC(C)C)CO2] | | | +++ | +++ |
| 111 | 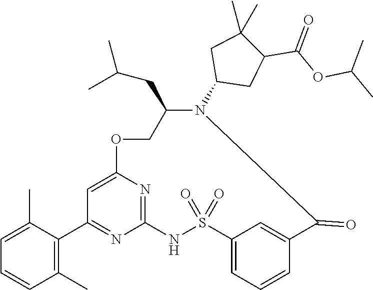 [SMILES: Cc1cccc(C)c1-c1cc2nc(n1)NS(=O)(=O)c1cccc(c1)C(=O)N([C@H]1CC(C(=O)OC(C)C)C(C)(C)C1)[C@H](CC(C)C)CO2] | | | | |
| 26 | 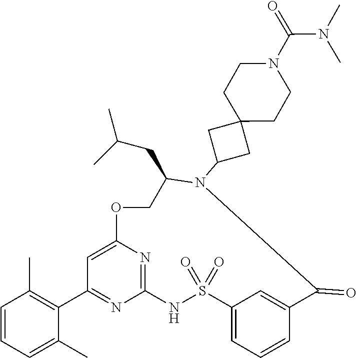 [SMILES: Cc1cccc(C)c1-c1cc2nc(n1)NS(=O)(=O)c1cccc(c1)C(=O)N(C1CC3(CCN(C(=O)N(C)C)CC3)C1)[C@H](CC(C)C)CO2] | | | +++ | +++ |
| 80 | 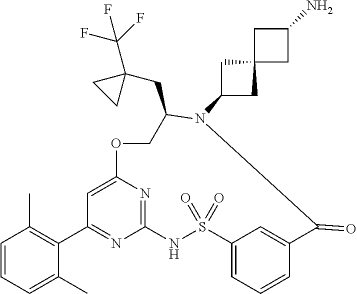 | | | +++ | +++ |
| 81 | 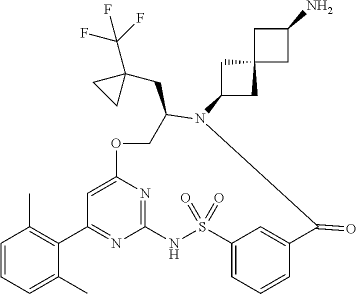 | | | +++ | +++ |
| 277 | 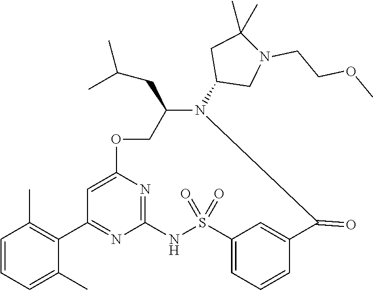 [SMILES: COCCN1C[C@H](N2C(=O)c3cccc(c3)S(=O)(=O)Nc3nc(cc(-c4c(C)cccc4C)n3)OC[C@H]2CC(C)C)CC1(C)C] | | | ++ | +++ |
| 424 | 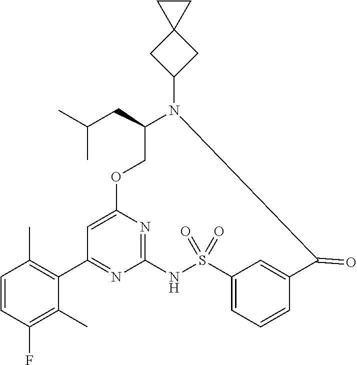 [SMILES: Cc1ccc(F)c(C)c1-c1cc2nc(n1)NS(=O)(=O)c1cccc(c1)C(=O)N(C1CC3(CC3)C1)[C@H](CC(C)C)CO2] | | | +++ | +++ |
| 423 | 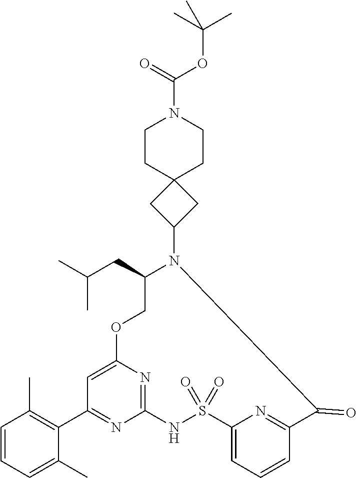 [SMILES: Cc1cccc(C)c1-c1cc2nc(n1)NS(=O)(=O)c1cccc(n1)C(=O)N(C1CC3(CCN(C(=O)OC(C)(C)C)CC3)C1)[C@H](CC(C)C)CO2] | | | +++ | +++ |
| 384 | 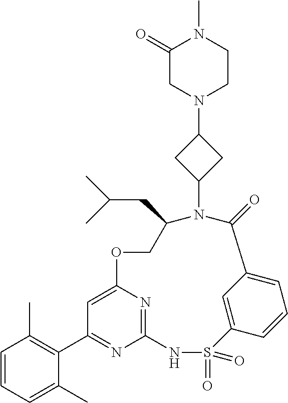 [SMILES: Cc1cccc(C)c1-c1cc2nc(n1)NS(=O)(=O)c1cccc(c1)C(=O)N(C1CC(N3CCN(C)C(=O)C3)C1)[C@H](CC(C)C)CO2] | | | +++ | +++ |
| 385 | 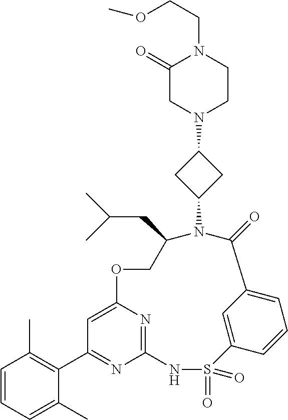 [SMILES: COCCN1CCN([C@H]2C[C@@H](N3C(=O)c4cccc(c4)S(=O)(=O)Nc4nc(cc(-c5c(C)cccc5C)n4)OC[C@H]3CC(C)C)C2)CC1=O] | | | +++ | +++ |
| 65 | 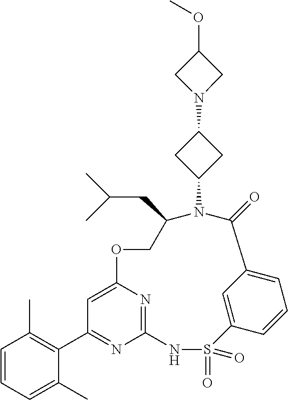 [SMILES: COC1CN([C@H]2C[C@@H](N3C(=O)c4cccc(c4)S(=O)(=O)Nc4nc(cc(-c5c(C)cccc5C)n4)OC[C@H]3CC(C)C)C2)C1] | | | +++ | +++ |
| 66 | 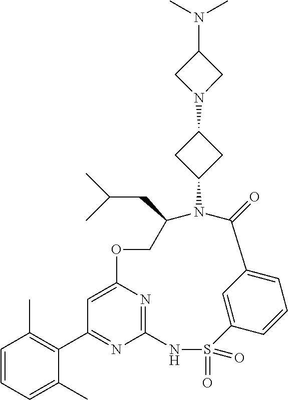 [SMILES: Cc1cccc(C)c1-c1cc2nc(n1)NS(=O)(=O)c1cccc(c1)C(=O)N([C@H]1C[C@@H](N3CC(N(C)C)C3)C1)[C@H](CC(C)C)CO2] | | | +++ | +++ |
| 383 | 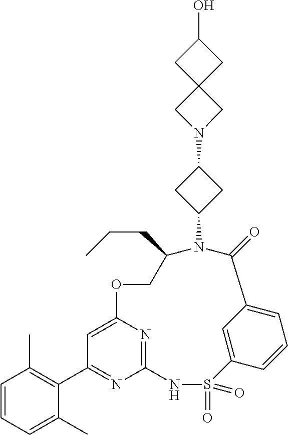 [SMILES: CCC[C@@H]1COc2cc(-c3c(C)cccc3C)nc(n2)NS(=O)(=O)c2cccc(c2)C(=O)N1[C@H]1C[C@@H](N2CC3(CC(O)C3)C2)C1] | | | +++ | +++ |
| 382 | 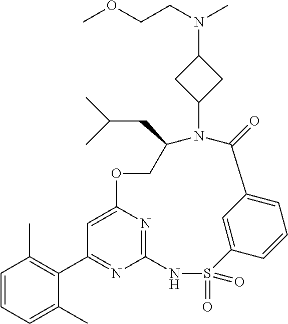 [SMILES: COCCN(C)C1CC(N2C(=O)c3cccc(c3)S(=O)(=O)Nc3nc(cc(-c4c(C)cccc4C)n3)OC[C@H]2CC(C)C)C1] | | | +++ | +++ |
| 213 | 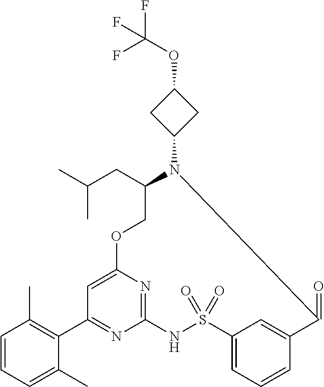 [SMILES: Cc1cccc(C)c1-c1cc2nc(n1)NS(=O)(=O)c1cccc(c1)C(=O)N([C@H]1C[C@@H](OC(F)(F)F)C1)[C@H](CC(C)C)CO2] | | | +++ | +++ |
| 214 | 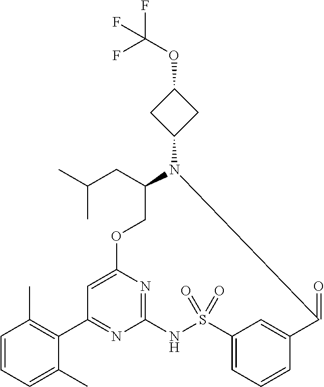 [SMILES: Cc1cccc(C)c1-c1cc2nc(n1)NS(=O)(=O)c1cccc(c1)C(=O)N([C@H]1C[C@@H](OC(F)(F)F)C1)[C@H](CC(C)C)CO2] | | | +++ | +++ |
| 268 | 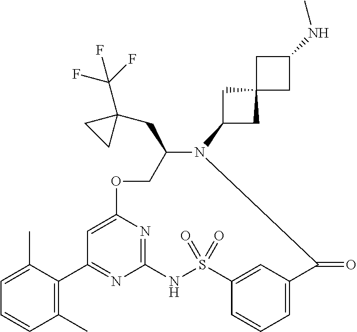 | | | +++ | +++ |
| 267 | 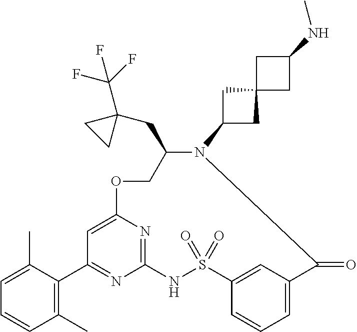 | | | +++ | +++ |
| 270 | 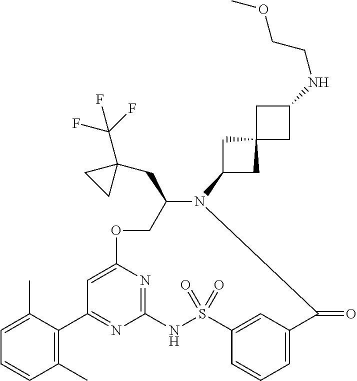 | | | +++ | +++ |
| 269 | 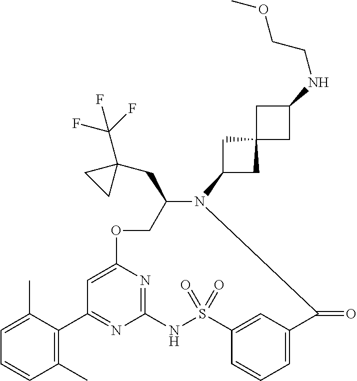 | | | +++ | +++ |
| 82 | 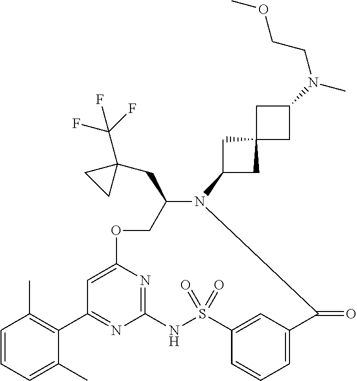 | | | +++ | +++ |
| 83 | 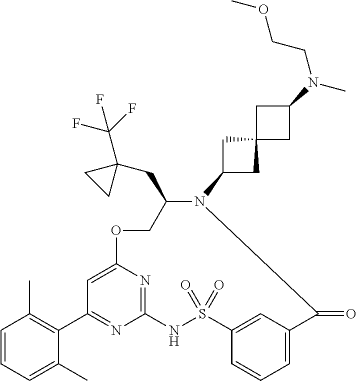 | | | +++ | +++ |
| 274 | 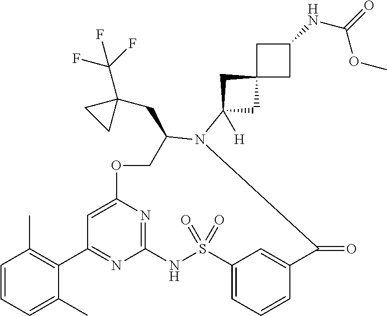 | | | +++ | +++ |
| 273 | 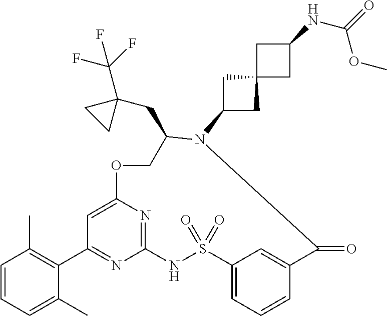 | | | +++ | +++ |
| 272 | 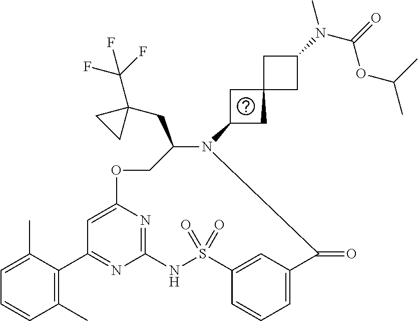 | | | +++ | +++ |
| 271 | 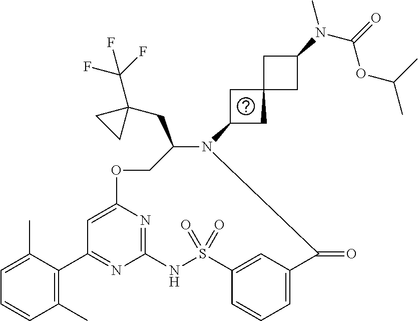 | | | +++ | +++ |
| 84 | 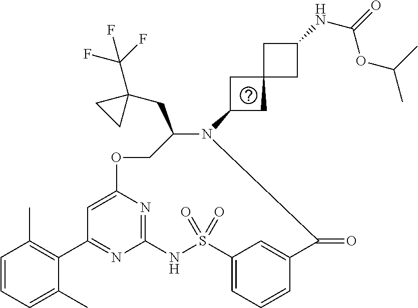 | | | +++ | +++ |
| 85 | 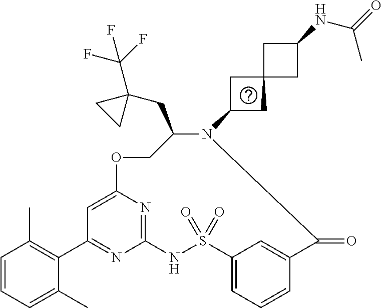 | | | +++ | +++ |
| 187 | 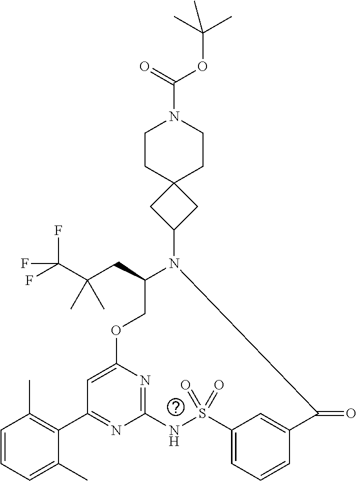 [SMILES: Cc1cccc(C)c1-c1cc2nc(n1)NS(=O)(=O)c1cccc(c1)C(=O)N(C1CC3(CCN(C(=O)OC(C)(C)C)CC3)C1)[C@H](CC(C)(C)C(F)(F)F)CO2] | | | +++ | +++ |
| 422 | 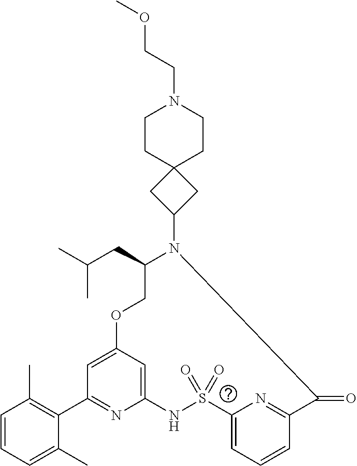 [SMILES: COCCN1CCC2(CC1)CC(N1C(=O)c3cccc(n3)S(=O)(=O)Nc3cc(cc(-c4c(C)cccc4C)n3)OC[C@H]1CC(C)C)C2] | | | +++ | +++ |
| 188 | 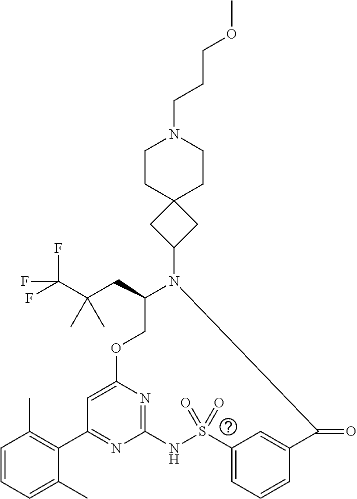 [SMILES: COCCCN1CCC2(CC1)CC(N1C(=O)c3cccc(c3)S(=O)(=O)Nc3nc(cc(-c4c(C)cccc4C)n3)OC[C@H]1CC(C)(C)C(F)(F)F)C2] | | | +++ | +++ |
| 189 | 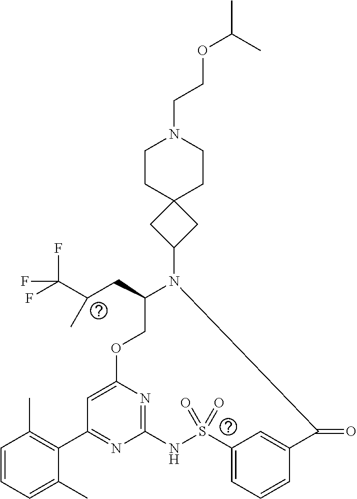 [SMILES: Cc1cccc(C)c1-c1cc2nc(n1)NS(=O)(=O)c1cccc(c1)C(=O)N(C1CC3(CCN(CCOC(C)C)CC3)C1)[C@H](CC(C)C(F)(F)F)CO2] | | | +++ | +++ |
| 421 | 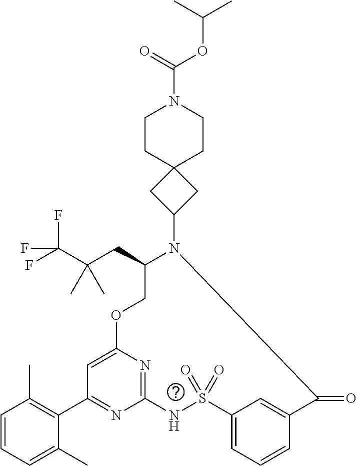 [SMILES: Cc1cccc(C)c1-c1cc2nc(n1)NS(=O)(=O)c1cccc(c1)C(=O)N(C1CC3(CCN(C(=O)OC(C)C)CC3)C1)[C@H](CC(C)(C)C(F)(F)F)CO2] | | | +++ | +++ |
| 67 | 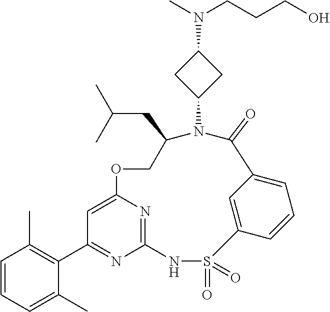 [SMILES: Cc1cccc(C)c1-c1cc2nc(n1)NS(=O)(=O)c1cccc(c1)C(=O)N([C@H]1C[C@@H](N(C)CCCO)C1)[C@H](CC(C)C)CO2] | | | +++ | +++ |
| 379 | 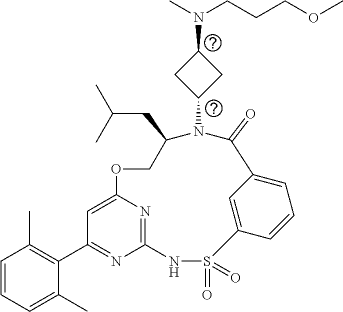 [SMILES: COCCCN(C)[C@H]1C[C@H](N2C(=O)c3cccc(c3)S(=O)(=O)Nc3nc(cc(-c4c(C)cccc4C)n3)OC[C@H]2CC(C)C)C1] | | | +++ | +++ |
| 380 | 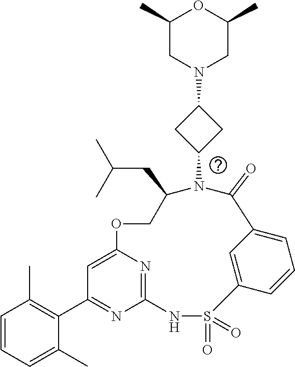 [SMILES: Cc1cccc(C)c1-c1cc2nc(n1)NS(=O)(=O)c1cccc(c1)C(=O)N([C@H]1C[C@@H](N3C[C@@H](C)O[C@@H](C)C3)C1)[C@H](CC(C)C)CO2] | | | +++ | +++ |
| 381 | 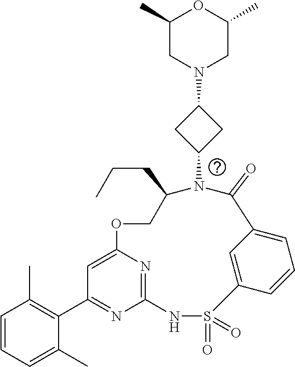 [SMILES: CCC[C@@H]1COc2cc(-c3c(C)cccc3C)nc(n2)NS(=O)(=O)c2cccc(c2)C(=O)N1[C@H]1C[C@@H](N2C[C@@H](C)O[C@H](C)C2)C1] | | | +++ | +++ |
| 191 | 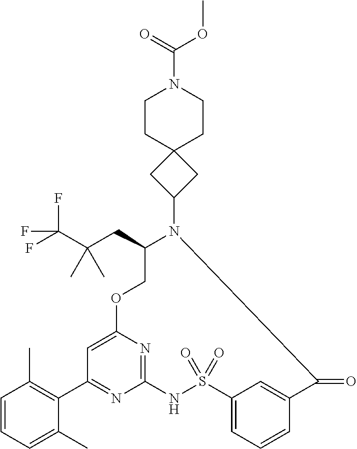 [SMILES: COC(=O)N1CCC2(CC1)CC(N1C(=O)c3cccc(c3)S(=O)(=O)Nc3nc(cc(-c4c(C)cccc4C)n3)OC[C@H]1CC(C)(C)C(F)(F)F)C2] | | | +++ | +++ |
| 190 | 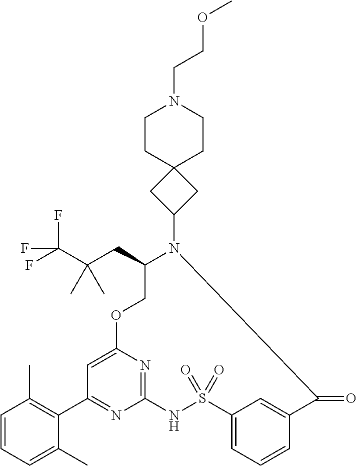 [SMILES: COCCN1CCC2(CC1)CC(N1C(=O)c3cccc(c3)S(=O)(=O)Nc3nc(cc(-c4c(C)cccc4C)n3)OC[C@H]1CC(C)(C)C(F)(F)F)C2] | | | +++ | +++ |
| 420 | 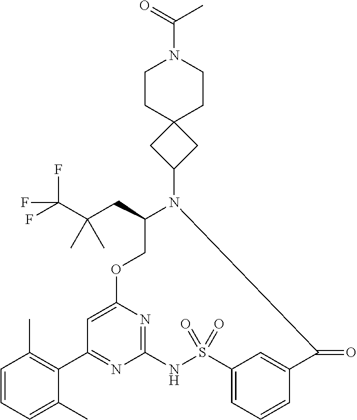 [SMILES: CC(=O)N1CCC2(CC1)CC(N1C(=O)c3cccc(c3)S(=O)(=O)Nc3nc(cc(-c4c(C)cccc4C)n3)OC[C@H]1CC(C)(C)C(F)(F)F)C2] | | | +++ | +++ |
| 378 | 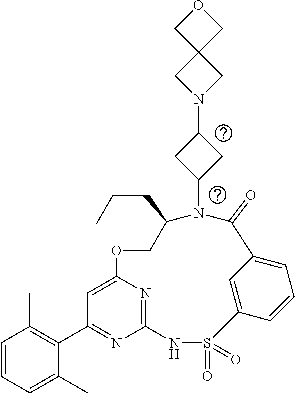 [SMILES: CCC[C@@H]1COc2cc(-c3c(C)cccc3C)nc(n2)NS(=O)(=O)c2cccc(c2)C(=O)N1C1CC(N2CC3(COC3)C2)C1] | | | ++ | +++ |
| 68 | 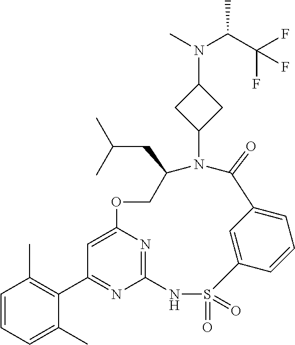 [SMILES: Cc1cccc(C)c1-c1cc2nc(n1)NS(=O)(=O)c1cccc(c1)C(=O)N(C1CC(N(C)[C@H](C)C(F)(F)F)C1)[C@H](CC(C)C)CO2] | | | +++ | ++ |
| 377 | 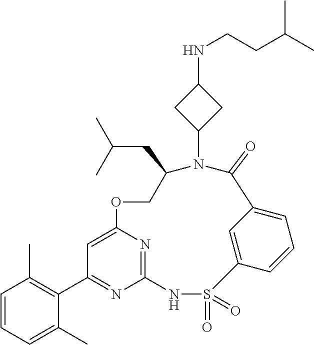 [SMILES: Cc1cccc(C)c1-c1cc2nc(n1)NS(=O)(=O)c1cccc(c1)C(=O)N(C1CC(NCCC(C)C)C1)[C@H](CC(C)C)CO2] | | | +++ | +++ |
| 69 | 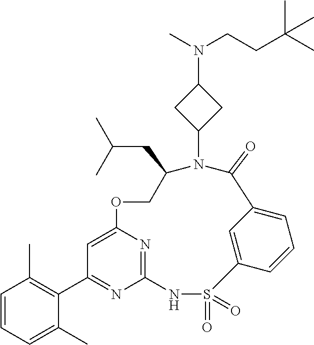 [SMILES: Cc1cccc(C)c1-c1cc2nc(n1)NS(=O)(=O)c1cccc(c1)C(=O)N(C1CC(N(C)CCC(C)(C)C)C1)[C@H](CC(C)C)CO2] | | | +++ | +++ |
| 375 | 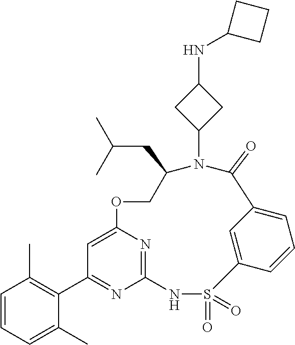 [SMILES: Cc1cccc(C)c1-c1cc2nc(n1)NS(=O)(=O)c1cccc(c1)C(=O)N(C1CC(NC3CCC3)C1)[C@H](CC(C)C)CO2] | | | +++ | +++ |
| 376 | 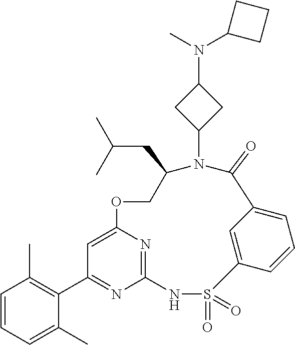 [SMILES: Cc1cccc(C)c1-c1cc2nc(n1)NS(=O)(=O)c1cccc(c1)C(=O)N(C1CC(N(C)C3CCC3)C1)[C@H](CC(C)C)CO2] | | | +++ | +++ |
| 373 | 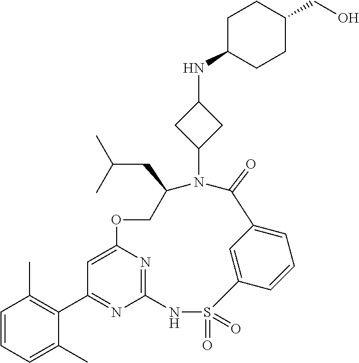 [SMILES: Cc1cccc(C)c1-c1cc2nc(n1)NS(=O)(=O)c1cccc(c1)C(=O)N(C1CC(N[C@H]3CC[C@H](CO)CC3)C1)[C@H](CC(C)C)CO2] | | | +++ | +++ |
| 374 | 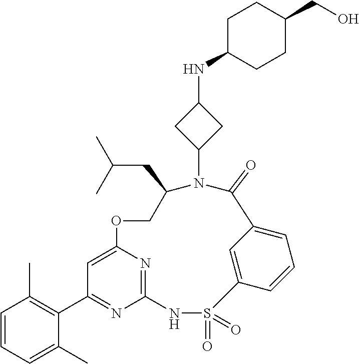 [SMILES: Cc1cccc(C)c1-c1cc2nc(n1)NS(=O)(=O)c1cccc(c1)C(=O)N(C1CC(N[C@H]3CC[C@@H](CO)CC3)C1)[C@H](CC(C)C)CO2] | | | +++ | +++ |
| 371 | 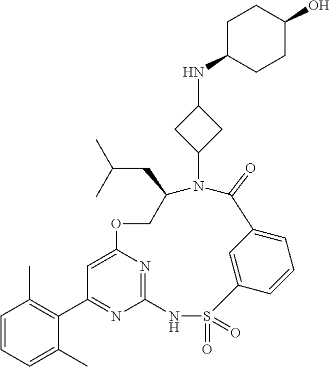 [SMILES: Cc1cccc(C)c1-c1cc2nc(n1)NS(=O)(=O)c1cccc(c1)C(=O)N(C1CC(N[C@H]3CC[C@@H](O)CC3)C1)[C@H](CC(C)C)CO2] | | | ++ | +++ |
| 372 | 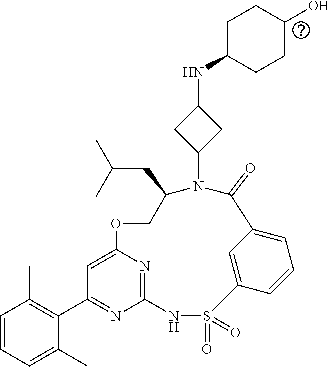 | | | +++ | +++ |
| 70 | 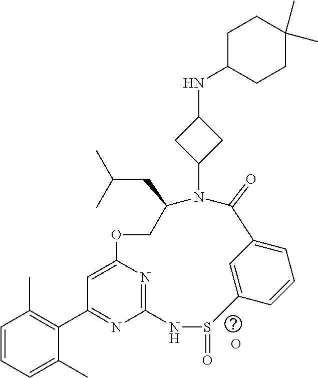 | | | +++ | +++ |
| 71 | 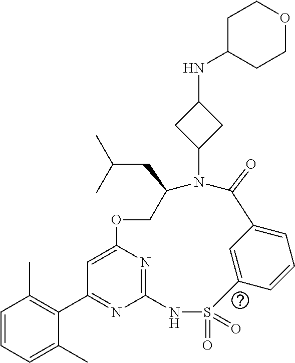 [SMILES: Cc1cccc(C)c1-c1cc2nc(n1)NS(=O)(=O)c1cccc(c1)C(=O)N(C1CC(NC3CCOCC3)C1)[C@H](CC(C)C)CO2] | | | +++ | +++ |
| 266 | 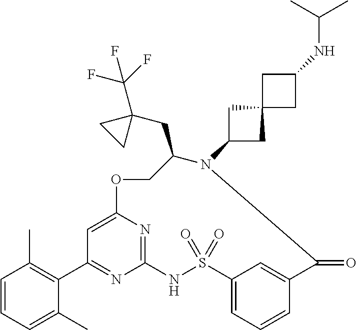 | | | +++ | +++ |
| 265 | 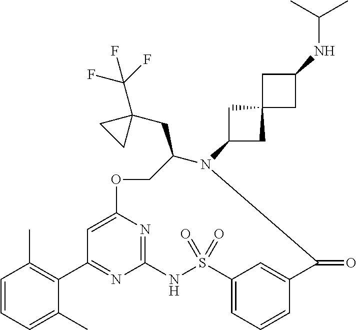 | | | +++ | +++ |
| 105 | 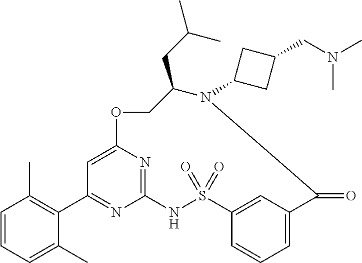 [SMILES: Cc1cccc(C)c1-c1cc2nc(n1)NS(=O)(=O)c1cccc(c1)C(=O)N([C@H]1C[C@@H](CN(C)C)C1)[C@H](CC(C)C)CO2] | | | +++ | +++ |
| 106 | 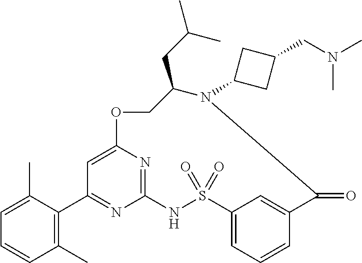 [SMILES: Cc1cccc(C)c1-c1cc2nc(n1)NS(=O)(=O)c1cccc(c1)C(=O)N([C@H]1C[C@@H](CN(C)C)C1)[C@H](CC(C)C)CO2] | | | +++ | +++ |
| 264 | 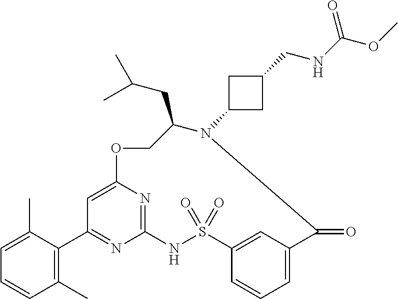 [SMILES: COC(=O)NC[C@H]1C[C@@H](N2C(=O)c3cccc(c3)S(=O)(=O)Nc3nc(cc(-c4c(C)cccc4C)n3)OC[C@H]2CC(C)C)C1] | | | +++ | +++ |
| 263 | 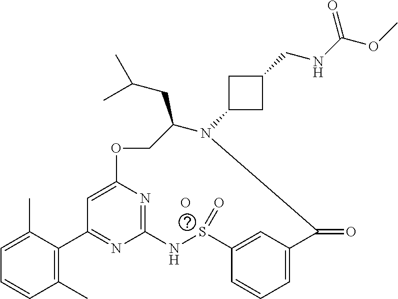 | | | +++ | +++ |
| 107 | 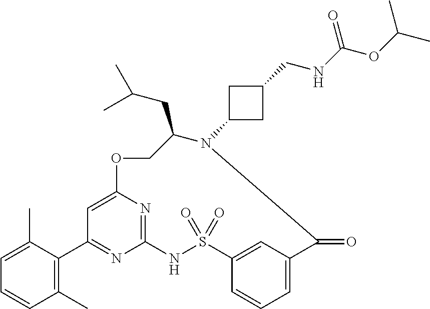 [SMILES: Cc1cccc(C)c1-c1cc2nc(n1)NS(=O)(=O)c1cccc(c1)C(=O)N([C@H]1C[C@@H](CNC(=O)OC(C)C)C1)[C@H](CC(C)C)CO2] | | | +++ | +++ |
| 108 | 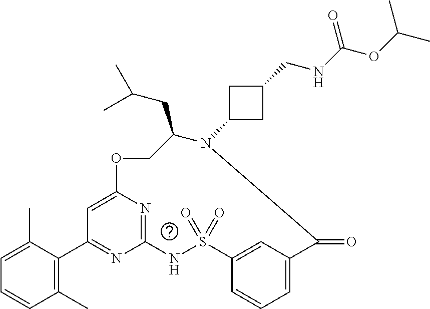 [SMILES: Cc1cccc(C)c1-c1cc2nc(n1)NS(=O)(=O)c1cccc(c1)C(=O)N([C@H]1C[C@@H](CNC(=O)OC(C)C)C1)[C@H](CC(C)C)CO2] | | | +++ | +++ |
| 262 | 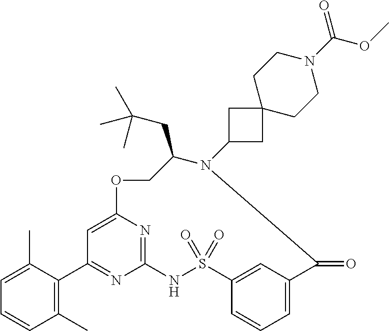 [SMILES: COC(=O)N1CCC2(CC1)CC(N1C(=O)c3cccc(c3)S(=O)(=O)Nc3nc(cc(-c4c(C)cccc4C)n3)OC[C@H]1CC(C)(C)C)C2] | | | +++ | +++ |
| 261 | 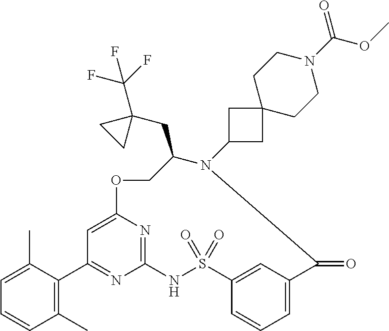 [SMILES: COC(=O)N1CCC2(CC1)CC(N1C(=O)c3cccc(c3)S(=O)(=O)Nc3nc(cc(-c4c(C)cccc4C)n3)OC[C@H]1CC1(C(F)(F)F)CC1)C2] | | | +++ | +++ |
| 75 | 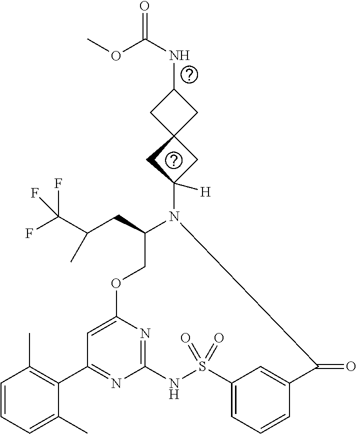 | | | +++ | +++ |
| 76 | 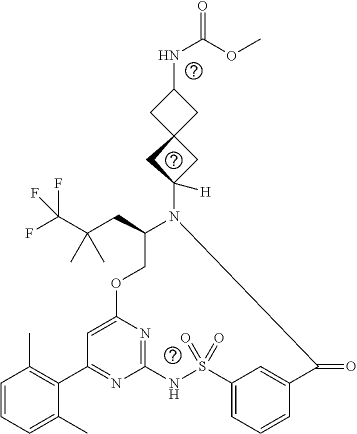 | | | +++ | +++ |
| 132 | 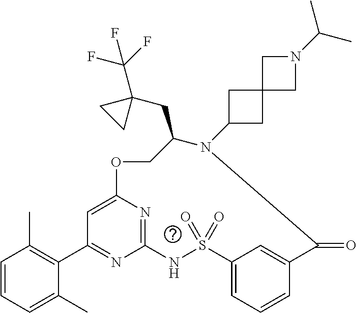 [SMILES: Cc1cccc(C)c1-c1cc2nc(n1)NS(=O)(=O)c1cccc(c1)C(=O)N(C1CC3(C1)CN(C(C)C)C3)[C@H](CC1(C(F)(F)F)CC1)CO2] | | | +++ | +++ |
| 186 | 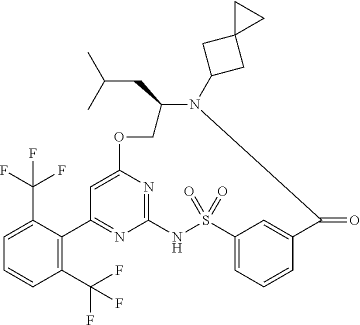 [SMILES: CC(C)C[C@@H]1COc2cc(-c3c(C(F)(F)F)cccc3C(F)(F)F)nc(n2)NS(=O)(=O)c2cccc(c2)C(=O)N1C1CC2(CC2)C1] | | | +++ | ++ |
| 72 | 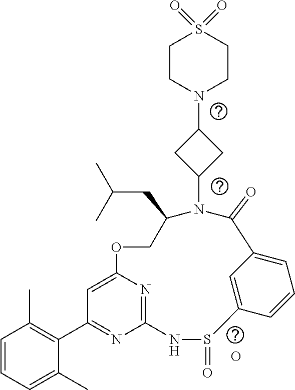 | | | +++ | +++ |
| 73 | 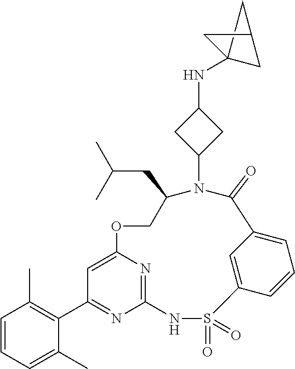 [SMILES: Cc1cccc(C)c1-c1cc2nc(n1)NS(=O)(=O)c1cccc(c1)C(=O)N(C1CC(NC34CC(C3)C4)C1)[C@H](CC(C)C)CO2] | | | +++ | +++ |
| 368 | 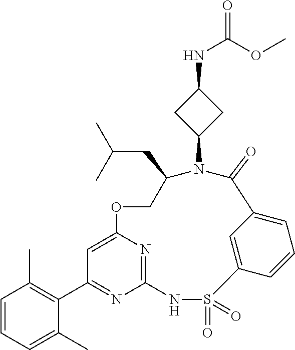 [SMILES: COC(=O)N[C@H]1C[C@@H](N2C(=O)c3cccc(c3)S(=O)(=O)Nc3nc(cc(-c4c(C)cccc4C)n3)OC[C@H]2CC(C)C)C1] | | | +++ | +++ |
| 367 | 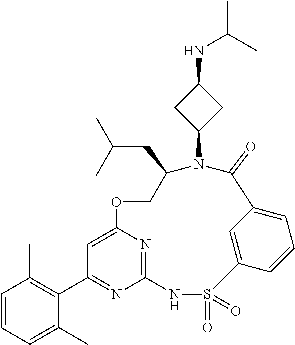 [SMILES: Cc1cccc(C)c1-c1cc2nc(n1)NS(=O)(=O)c1cccc(c1)C(=O)N([C@H]1C[C@@H](NC(C)C)C1)[C@H](CC(C)C)CO2] | | | +++ | +++ |
| 366 | 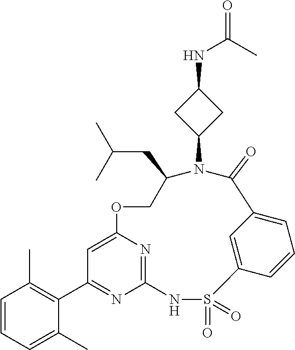 [SMILES: CC(=O)N[C@H]1C[C@@H](N2C(=O)c3cccc(c3)S(=O)(=O)Nc3nc(cc(-c4c(C)cccc4C)n3)OC[C@H]2CC(C)C)C1] | | | +++ | +++ |
| 168 | 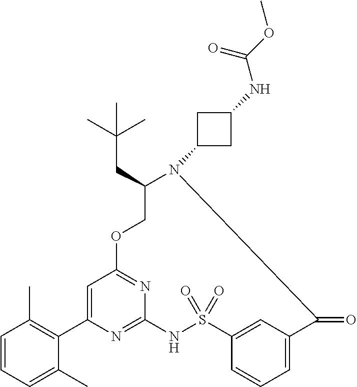 [SMILES: COC(=O)N[C@H]1C[C@@H](N2C(=O)c3cccc(c3)S(=O)(=O)Nc3nc(cc(-c4c(C)cccc4C)n3)OC[C@H]2CC(C)(C)C)C1] | | | +++ | +++ |
| 453 | 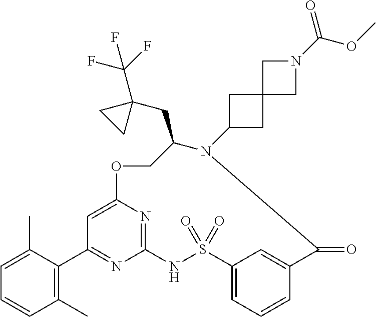 [SMILES: COC(=O)N1CC2(CC(N3C(=O)c4cccc(c4)S(=O)(=O)Nc4nc(cc(-c5c(C)cccc5C)n4)OC[C@H]3CC3(C(F)(F)F)CC3)C2)C1] | | | +++ | +++ |
| 133 | 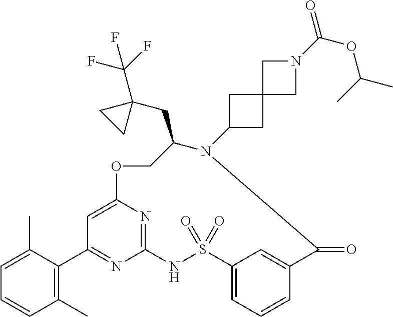 [SMILES: Cc1cccc(C)c1-c1cc2nc(n1)NS(=O)(=O)c1cccc(c1)C(=O)N(C1CC3(C1)CN(C(=O)OC(C)C)C3)[C@H](CC1(C(F)(F)F)CC1)CO2] | | | +++ | +++ |
| 452 | 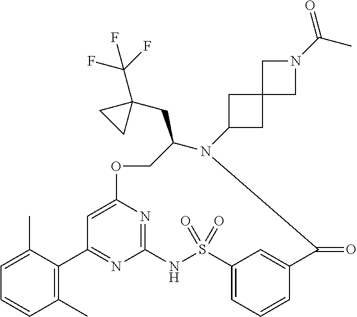 [SMILES: CC(=O)N1CC2(CC(N3C(=O)c4cccc(c4)S(=O)(=O)Nc4nc(cc(-c5c(C)cccc5C)n4)OC[C@H]3CC3(C(F)(F)F)CC3)C2)C1] | | | +++ | +++ |
| 472 | 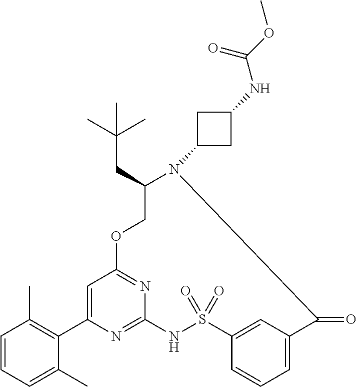 [SMILES: COC(=O)N[C@H]1C[C@@H](N2C(=O)c3cccc(c3)S(=O)(=O)Nc3nc(cc(-c4c(C)cccc4C)n3)OC[C@H]2CC(C)(C)C)C1] | | | +++ | +++ |
| 451 | 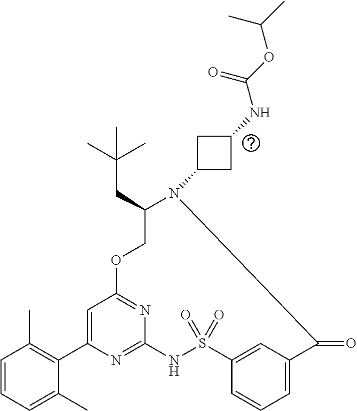 [SMILES: Cc1cccc(C)c1-c1cc2nc(n1)NS(=O)(=O)c1cccc(c1)C(=O)N([C@H]1C[C@@H](NC(=O)OC(C)C)C1)[C@H](CC(C)(C)C)CO2] | | | +++ | +++ |
| 450 | 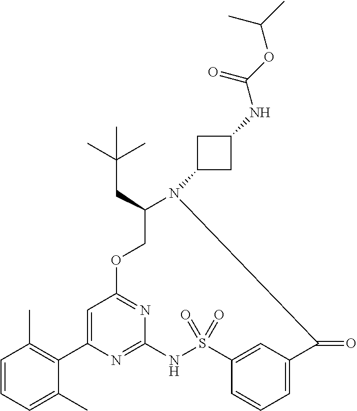 [SMILES: Cc1cccc(C)c1-c1cc2nc(n1)NS(=O)(=O)c1cccc(c1)C(=O)N([C@H]1C[C@@H](NC(=O)OC(C)C)C1)[C@H](CC(C)(C)C)CO2] | | | +++ | +++ |
| 449 | 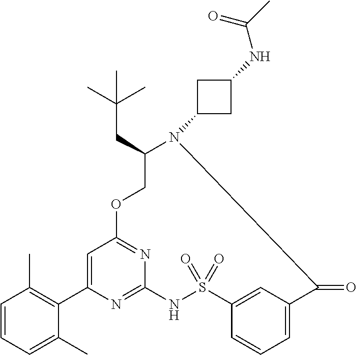 [SMILES: CC(=O)N[C@H]1C[C@@H](N2C(=O)c3cccc(c3)S(=O)(=O)Nc3nc(cc(-c4c(C)cccc4C)n3)OC[C@H]2CC(C)(C)C)C1] | | | +++ | +++ |
| 448 | 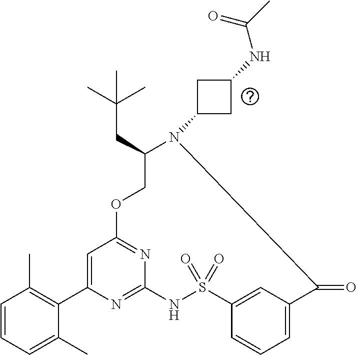 [SMILES: CC(=O)N[C@H]1C[C@@H](N2C(=O)c3cccc(c3)S(=O)(=O)Nc3nc(cc(-c4c(C)cccc4C)n3)OC[C@H]2CC(C)(C)C)C1] | | | +++ | +++ |
| 54 | 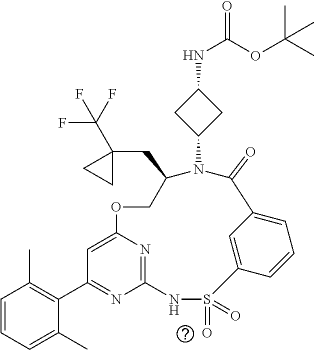 [SMILES: Cc1cccc(C)c1-c1cc2nc(n1)NS(=O)(=O)c1cccc(c1)C(=O)N([C@H]1C[C@@H](NC(=O)OC(C)(C)C)C1)[C@H](CC1(C(F)(F)F)CC1)CO2] | | | +++ | +++ |
| 55 | 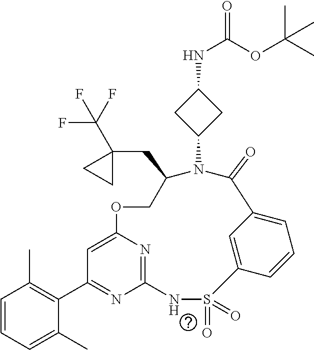 [SMILES: Cc1cccc(C)c1-c1cc2nc(n1)NS(=O)(=O)c1cccc(c1)C(=O)N([C@H]1C[C@@H](NC(=O)OC(C)(C)C)C1)[C@H](CC1(C(F)(F)F)CC1)CO2] | | | +++ | +++ |
| 56 | 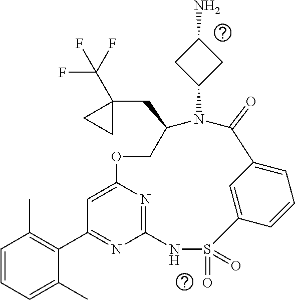 [SMILES: Cc1cccc(C)c1-c1cc2nc(n1)NS(=O)(=O)c1cccc(c1)C(=O)N([C@H]1C[C@@H](N)C1)[C@H](CC1(C(F)(F)F)CC1)CO2] | | | +++ | +++ |
| 57 | 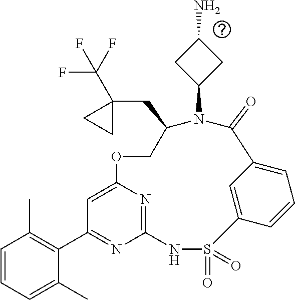 [SMILES: Cc1cccc(C)c1-c1cc2nc(n1)NS(=O)(=O)c1cccc(c1)C(=O)N([C@H]1C[C@H](N)C1)[C@H](CC1(C(F)(F)F)CC1)CO2] | | | +++ | +++ |
| 53 | 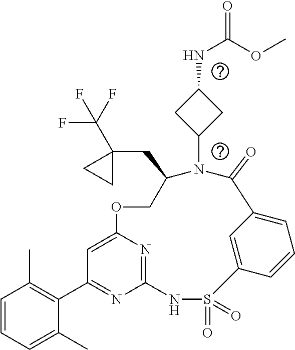 | | | +++ | +++ |
| 58 | 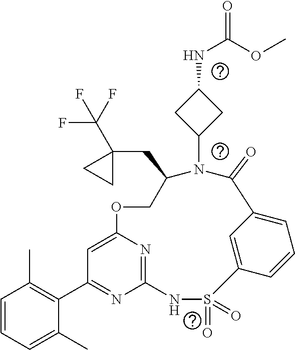 | | | +++ | +++ |
| 59 | 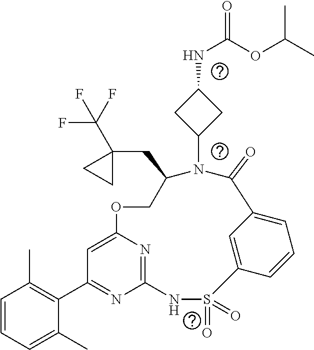 | | | +++ | +++ |
| 60 | 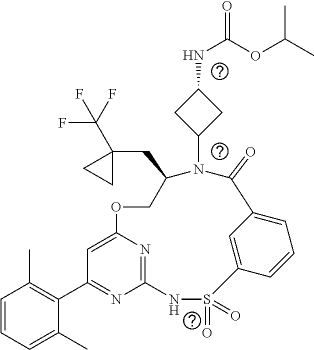 | | | +++ | +++ |
| 447 | 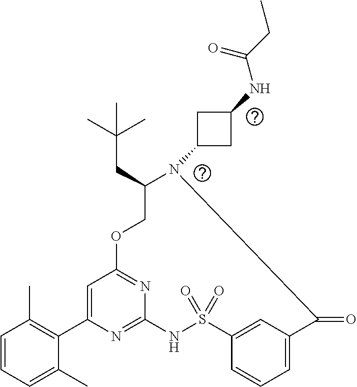 [SMILES: CCC(=O)N[C@H]1C[C@H](N2C(=O)c3cccc(c3)S(=O)(=O)Nc3nc(cc(-c4c(C)cccc4C)n3)OC[C@H]2CC(C)(C)C)C1] | | | +++ | +++ |
| 446 | 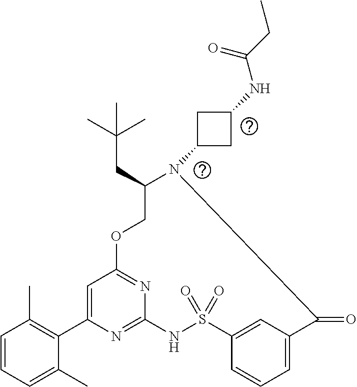 [SMILES: CCC(=O)N[C@H]1C[C@@H](N2C(=O)c3cccc(c3)S(=O)(=O)Nc3nc(cc(-c4c(C)cccc4C)n3)OC[C@H]2CC(C)(C)C)C1] | | | +++ | +++ |
| 445 | 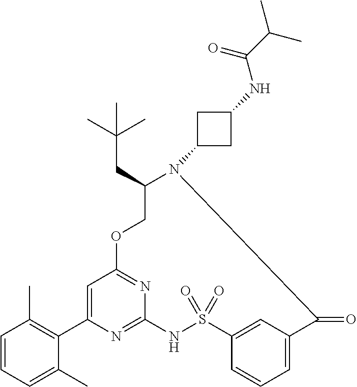 [SMILES: Cc1cccc(C)c1-c1cc2nc(n1)NS(=O)(=O)c1cccc(c1)C(=O)N([C@H]1C[C@@H](NC(=O)C(C)C)C1)[C@H](CC(C)(C)C)CO2] | | | +++ | +++ |
| 444 | 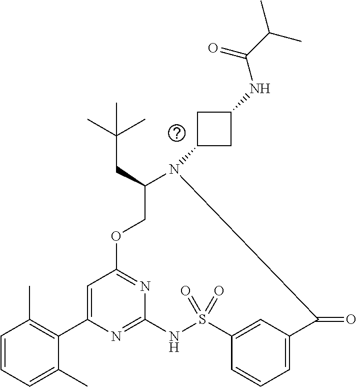 [SMILES: Cc1cccc(C)c1-c1cc2nc(n1)NS(=O)(=O)c1cccc(c1)C(=O)N([C@H]1C[C@@H](NC(=O)C(C)C)C1)[C@H](CC(C)(C)C)CO2] | | | +++ | +++ |
| 260 | 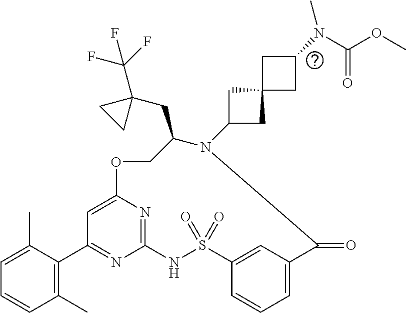 | | | +++ | +++ |
| 259 | 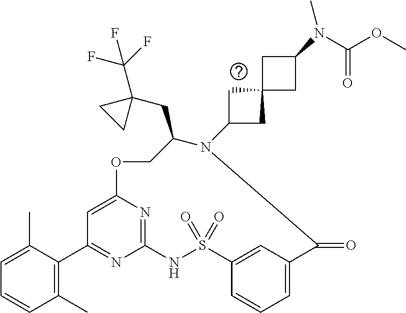 | | | +++ | +++ |
| 257 | 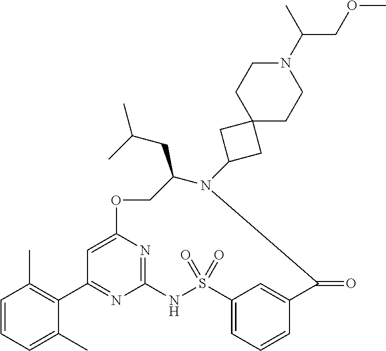 [SMILES: COCC(C)N1CCC2(CC1)CC(N1C(=O)c3cccc(c3)S(=O)(=O)Nc3nc(cc(-c4c(C)cccc4C)n3)OC[C@H]1CC(C)C)C2] | | | +++ | +++ |
| 258 | 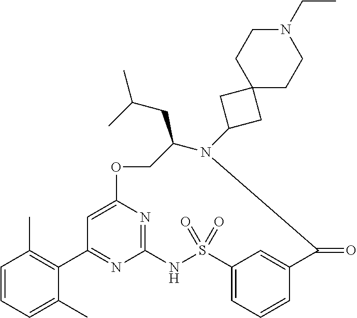 [SMILES: CCN1CCC2(CC1)CC(N1C(=O)c3cccc(c3)S(=O)(=O)Nc3nc(cc(-c4c(C)cccc4C)n3)OC[C@H]1CC(C)C)C2] | | | +++ | +++ |
| 193 | 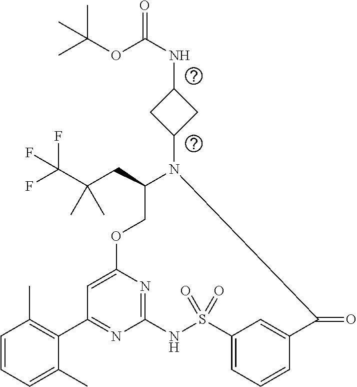 [SMILES: Cc1cccc(C)c1-c1cc2nc(n1)NS(=O)(=O)c1cccc(c1)C(=O)N(C1CC(NC(=O)OC(C)(C)C)C1)[C@H](CC(C)(C)C(F)(F)F)CO2] | | | +++ | +++ |
| 194 | 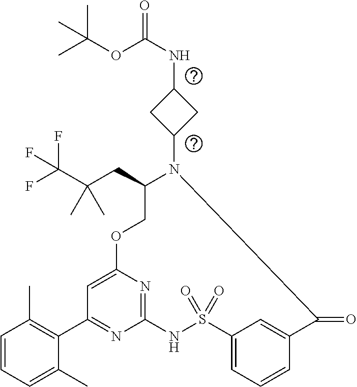 [SMILES: Cc1cccc(C)c1-c1cc2nc(n1)NS(=O)(=O)c1cccc(c1)C(=O)N(C1CC(NC(=O)OC(C)(C)C)C1)[C@H](CC(C)(C)C(F)(F)F)CO2] | | | +++ | +++ |
| 418 | 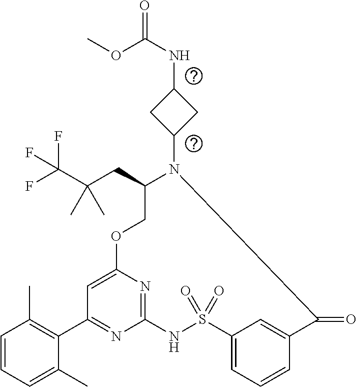 [SMILES: COC(=O)NC1CC(N2C(=O)c3cccc(c3)S(=O)(=O)Nc3nc(cc(-c4c(C)cccc4C)n3)OC[C@H]2CC(C)(C)C(F)(F)F)C1] | | | +++ | +++ |

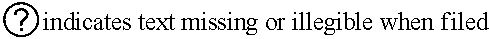

Table 10 represents CFTR modulating activity for representative compounds of the disclosure generated using one or more of the assays described in this example (EC50: +++ is <1 μM; ++ is 1-<3 μM; + is 3-<30 μM; and ND is “not detected in this assay.” 0% Activity: +++ is >60%; ++ is 30-60%; + is <300%).

TABLE 10
Biological Activity Data

| | | | Ent. A | | Ent. B |
|---|---|---|---|---|---|
| | | Ent. A | Max | Ent. B | Max |
| Cmpd | | EC₅₀ | Activity | EC₅₀ | Activity |
| No. | Structure | (μM) | (%) | (μM) | (%) |
| 419 | 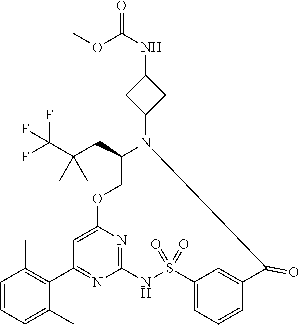 [SMILES: COC(=O)NC1CC(N2C(=O)c3cccc(c3)S(=O)(=O)Nc3nc(cc(-c4c(C)cccc4C)n3)OC[C@H]2CC(C)(C)C(F)(F)F)C1] | | | +++ | +++ |
| 417 | 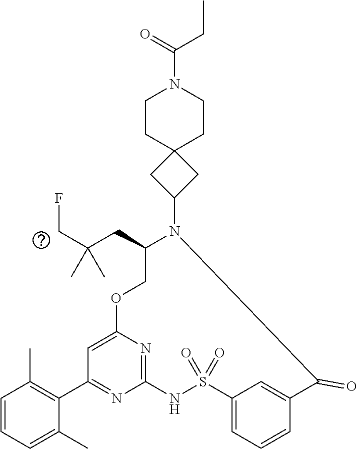 [SMILES: CCC(=O)N1CCC2(CC1)CC(N1C(=O)c3cccc(c3)S(=O)(=O)Nc3nc(cc(-c4c(C)cccc4C)n3)OC[C@H]1CC(C)(C)CF)C2] | | | +++ | +++ |
| 416 | 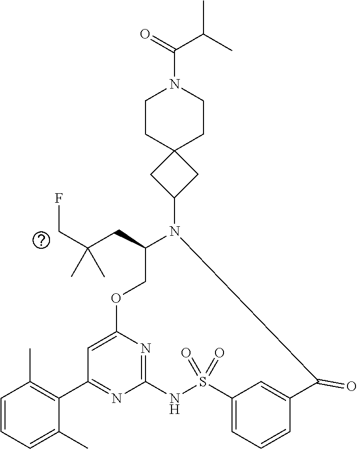 [SMILES: Cc1cccc(C)c1-c1cc2nc(n1)NS(=O)(=O)c1cccc(c1)C(=O)N(C1CC3(CCN(C(=O)C(C)C)CC3)C1)[C@H](CC(C)(C)CF)CO2] | | | +++ | +++ |
| 165 | 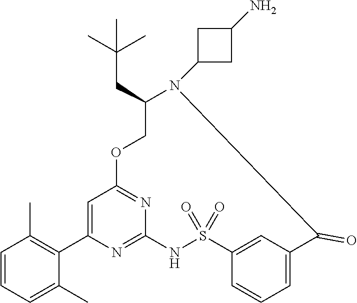 [SMILES: Cc1cccc(C)c1-c1cc2nc(n1)NS(=O)(=O)c1cccc(c1)C(=O)N(C1CC(N)C1)[C@H](CC(C)(C)C)CO2] | | | +++ | ++ |
| 166 | 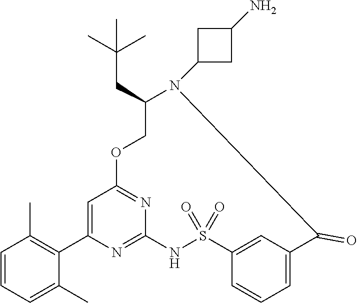 [SMILES: Cc1cccc(C)c1-c1cc2nc(n1)NS(=O)(=O)c1cccc(c1)C(=O)N(C1CC(N)C1)[C@H](CC(C)(C)C)CO2] | | | +++ | ++ |
| 192 | 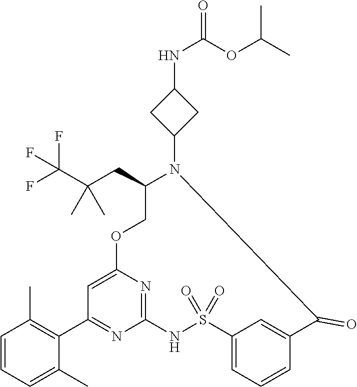 [SMILES: Cc1cccc(C)c1-c1cc2nc(n1)NS(=O)(=O)c1cccc(c1)C(=O)N(C1CC(NC(=O)OC(C)C)C1)[C@H](CC(C)(C)C(F)(F)F)CO2] | | | +++ | +++ |
| 195 | 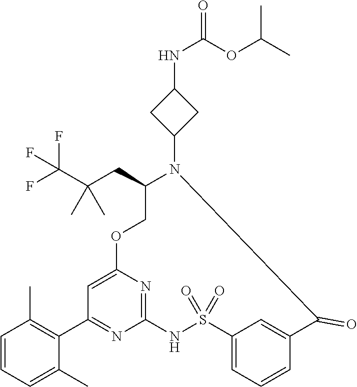 [SMILES: Cc1cccc(C)c1-c1cc2nc(n1)NS(=O)(=O)c1cccc(c1)C(=O)N(C1CC(NC(=O)OC(C)C)C1)[C@H](CC(C)(C)C(F)(F)F)CO2] | | | +++ | ++ |
| 200 | 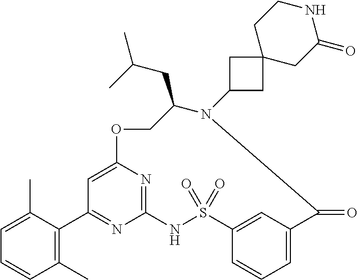 [SMILES: Cc1cccc(C)c1-c1cc2nc(n1)NS(=O)(=O)c1cccc(c1)C(=O)N(C1CC3(CCNC(=O)C3)C1)[C@H](CC(C)C)CO2] | | | +++ | ++ |
| 201 | 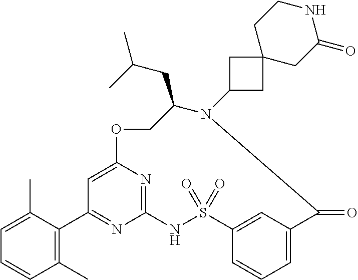 [SMILES: Cc1cccc(C)c1-c1cc2nc(n1)NS(=O)(=O)c1cccc(c1)C(=O)N(C1CC3(CCNC(=O)C3)C1)[C@H](CC(C)C)CO2] | | | +++ | ++ |
| 169 | 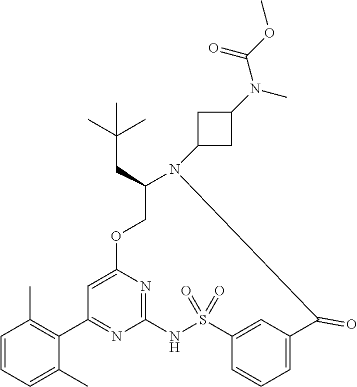 [SMILES: COC(=O)N(C)C1CC(N2C(=O)c3cccc(c3)S(=O)(=O)Nc3nc(cc(-c4c(C)cccc4C)n3)OC[C@H]2CC(C)(C)C)C1] | | | +++ | +++ |
| 170 | 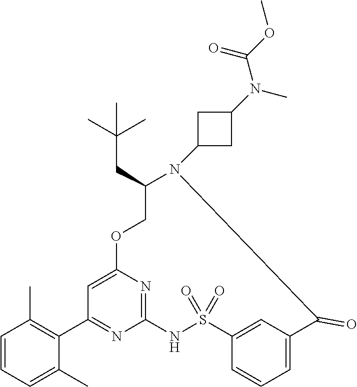 [SMILES: COC(=O)N(C)C1CC(N2C(=O)c3cccc(c3)S(=O)(=O)Nc3nc(cc(-c4c(C)cccc4C)n3)OC[C@H]2CC(C)(C)C)C1] | | | | ++ |
| 254 | 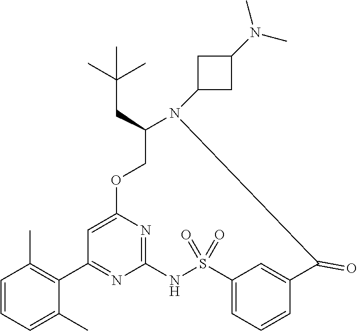 [SMILES: Cc1cccc(C)c1-c1cc2nc(n1)NS(=O)(=O)c1cccc(c1)C(=O)N(C1CC(N(C)C)C1)[C@H](CC(C)(C)C)CO2] | | | +++ | ++ |
| 253 | 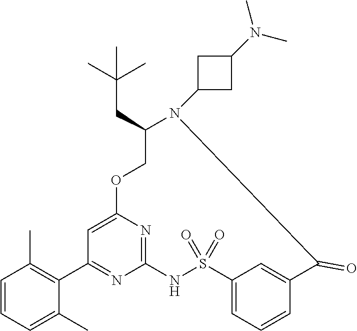 [SMILES: Cc1cccc(C)c1-c1cc2nc(n1)NS(=O)(=O)c1cccc(c1)C(=O)N(C1CC(N(C)C)C1)[C@H](CC(C)(C)C)CO2] | | | +++ | ++ |
| 255 | 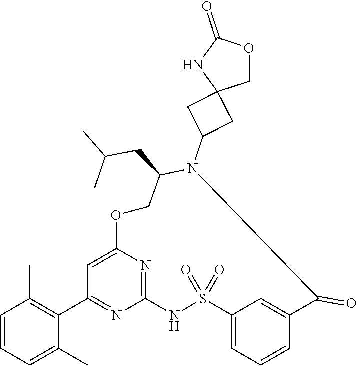 [SMILES: Cc1cccc(C)c1-c1cc2nc(n1)NS(=O)(=O)c1cccc(c1)C(=O)N(C1CC3(COC(=O)N3)C1)[C@H](CC(C)C)CO2] | | | +++ | ++ |
| 256 | 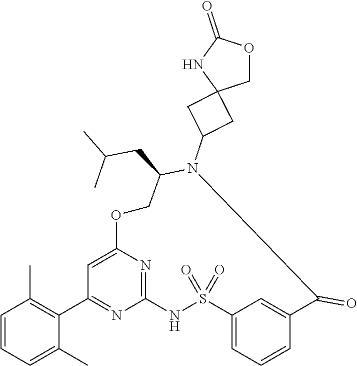 [SMILES: Cc1cccc(C)c1-c1cc2nc(n1)NS(=O)(=O)c1cccc(c1)C(=O)N(C1CC3(COC(=O)N3)C1)[C@H](CC(C)C)CO2] | | | +++ | ++ |
| 61 | 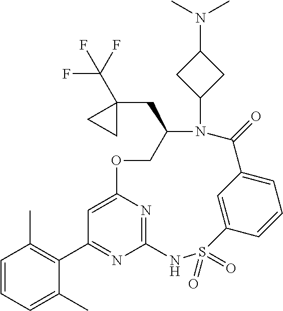 [SMILES: Cc1cccc(C)c1-c1cc2nc(n1)NS(=O)(=O)c1cccc(c1)C(=O)N(C1CC(N(C)C)C1)[C@H](CC1(C(F)(F)F)CC1)CO2] | | | +++ | +++ |
| 360 | 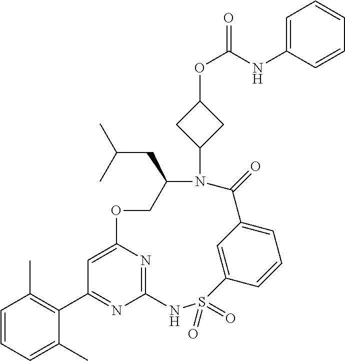 [SMILES: Cc1cccc(C)c1-c1cc2nc(n1)NS(=O)(=O)c1cccc(c1)C(=O)N(C1CC(OC(=O)Nc3ccccc3)C1)[C@H](CC(C)C)CO2] | | | +++ | +++ |
| 362 | 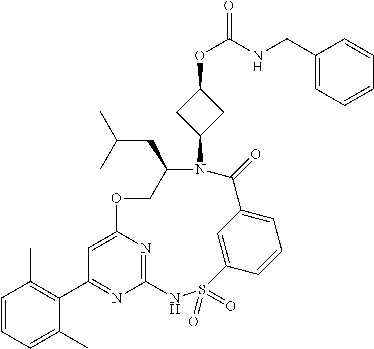 [SMILES: Cc1cccc(C)c1-c1cc2nc(n1)NS(=O)(=O)c1cccc(c1)C(=O)N([C@H]1C[C@@H](OC(=O)NCc3ccccc3)C1)[C@H](CC(C)C)CO2] | | | +++ | ++ |
| 363 | 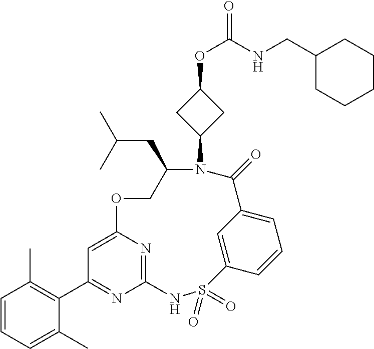 [SMILES: Cc1cccc(C)c1-c1cc2nc(n1)NS(=O)(=O)c1cccc(c1)C(=O)N([C@H]1C[C@@H](OC(=O)NCC3CCCCC3)C1)[C@H](CC(C)C)CO2] | | | +++ | ++ |
| 364 | 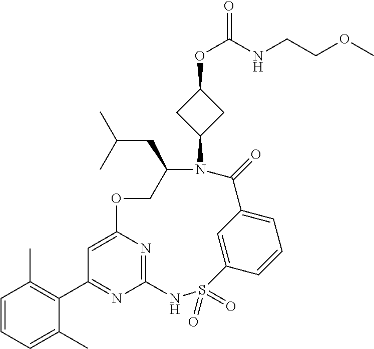 [SMILES: COCCNC(=O)O[C@H]1C[C@@H](N2C(=O)c3cccc(c3)S(=O)(=O)Nc3nc(cc(-c4c(C)cccc4C)n3)OC[C@H]2CC(C)C)C1] | | | +++ | +++ |
| 365 | 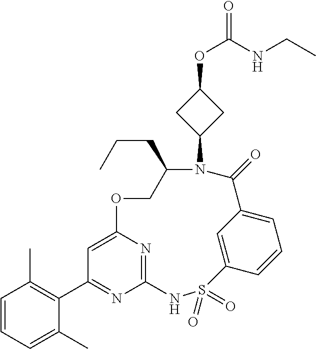 [SMILES: CCC[C@@H]1COc2cc(-c3c(C)cccc3C)nc(n2)NS(=O)(=O)c2cccc(c2)C(=O)N1[C@H]1C[C@@H](OC(=O)NCC)C1] | | | +++ | ++ |
| 202 | 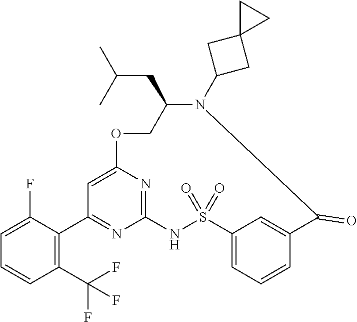 [SMILES: CC(C)C[C@@H]1COc2cc(-c3c(F)cccc3C(F)(F)F)nc(n2)NS(=O)(=O)c2cccc(c2)C(=O)N1C1CC2(CC2)C1] | | | +++ | ++ |
| 361 | 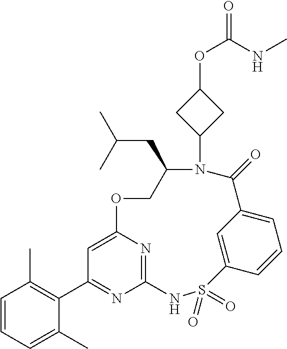 [SMILES: CNC(=O)OC1CC(N2C(=O)c3cccc(c3)S(=O)(=O)Nc3nc(cc(-c4c(C)cccc4C)n3)OC[C@H]2CC(C)C)C1] | | | +++ | ++ |
| 369 | 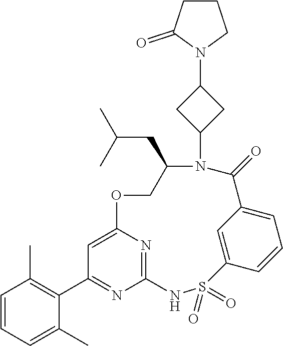 [SMILES: Cc1cccc(C)c1-c1cc2nc(n1)NS(=O)(=O)c1cccc(c1)C(=O)N(C1CC(N3CCCC3=O)C1)[C@H](CC(C)C)CO2] | | | +++ | ++ |
| 370 | 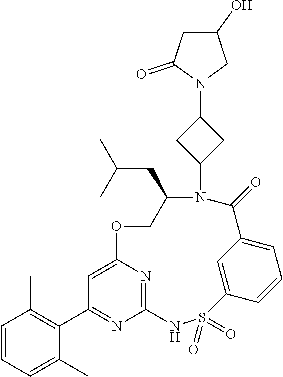 [SMILES: Cc1cccc(C)c1-c1cc2nc(n1)NS(=O)(=O)c1cccc(c1)C(=O)N(C1CC(N3CC(O)CC3=O)C1)[C@H](CC(C)C)CO2] | | | +++ | ++ |
| 251 | 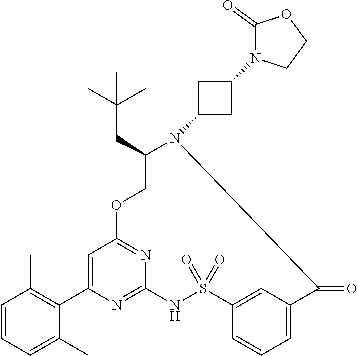 [SMILES: Cc1cccc(C)c1-c1cc2nc(n1)NS(=O)(=O)c1cccc(c1)C(=O)N([C@H]1C[C@@H](N3CCOC3=O)C1)[C@H](CC(C)(C)C)CO2] | | | +++ | +++ |
| 167 | 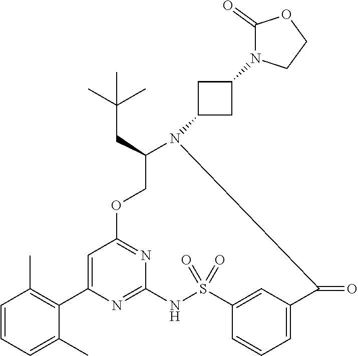 [SMILES: Cc1cccc(C)c1-c1cc2nc(n1)NS(=O)(=O)c1cccc(c1)C(=O)N([C@H]1C[C@@H](N3CCOC3=O)C1)[C@H](CC(C)(C)C)CO2] | | | +++ | ++ |
| 252 | 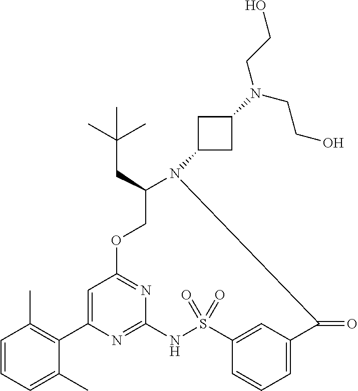 [SMILES: Cc1cccc(C)c1-c1cc2nc(n1)NS(=O)(=O)c1cccc(c1)C(=O)N([C@H]1C[C@@H](N(CCO)CCO)C1)[C@H](CC(C)(C)C)CO2] | | | +++ | + |
| 250 | 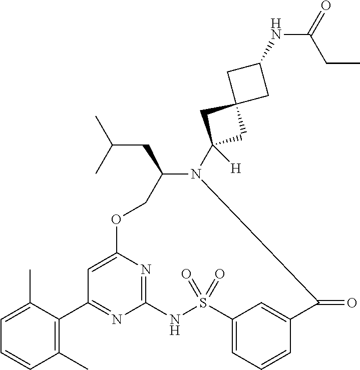 | | | +++ | |
| 249 | 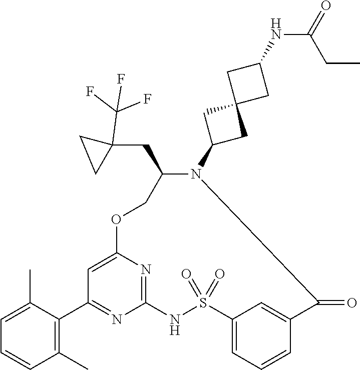 | | | +++ | +++ |
| 248 | 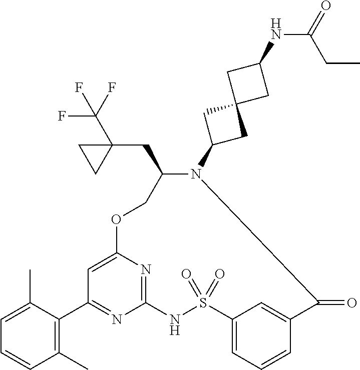 | | | +++ | +++ |
| 359 | 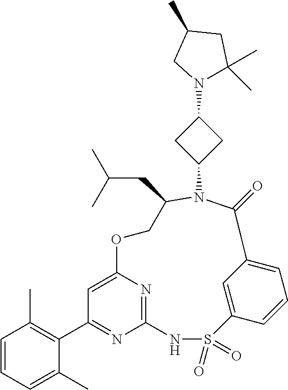 [SMILES: Cc1cccc(C)c1-c1cc2nc(n1)NS(=O)(=O)c1cccc(c1)C(=O)N([C@H]1C[C@@H](N3C[C@@H](C)CC3(C)C)C1)[C@H](CC(C)C)CO2] | | | +++ | ++ |
| 212 | 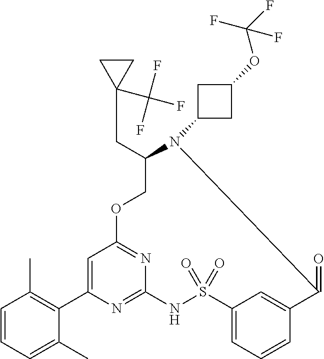 [SMILES: Cc1cccc(C)c1-c1cc2nc(n1)NS(=O)(=O)c1cccc(c1)C(=O)N([C@H]1C[C@@H](OC(F)(F)F)C1)[C@H](CC1(C(F)(F)F)CC1)CO2] | | | +++ | ++ |
| 199 | 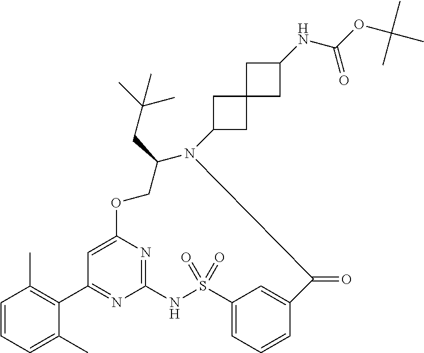 [SMILES: Cc1cccc(C)c1-c1cc2nc(n1)NS(=O)(=O)c1cccc(c1)C(=O)N(C1CC3(CC(NC(=O)OC(C)(C)C)C3)C1)[C@H](CC(C)(C)C)CO2] | | | +++ | +++ |
| 175 | 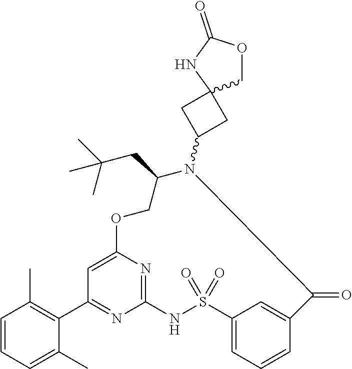 [SMILES: Cc1cccc(C)c1-c1cc2nc(n1)NS(=O)(=O)c1cccc(c1)C(=O)N(C1CC3(COC(=O)N3)C1)[C@H](CC(C)(C)C)CO2] | | | +++ | ++ |
| 176 | 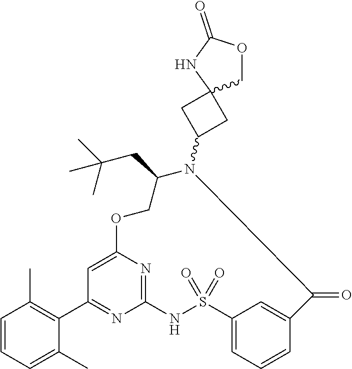 [SMILES: Cc1cccc(C)c1-c1cc2nc(n1)NS(=O)(=O)c1cccc(c1)C(=O)N(C1CC3(COC(=O)N3)C1)[C@H](CC(C)(C)C)CO2] | | | +++ | ++ |
| 123 | 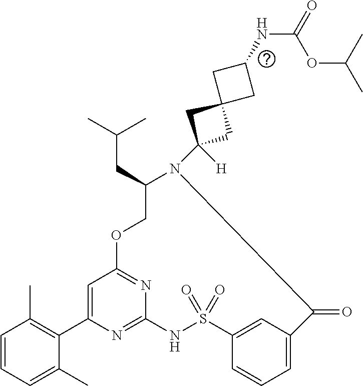 | | | +++ | +++ |
| 247 | 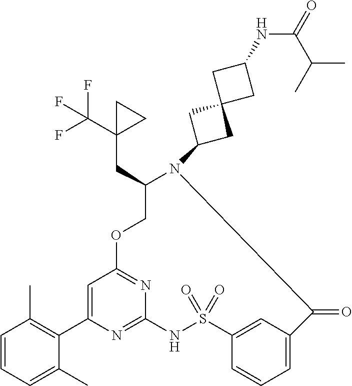 | | | +++ | +++ |
| 246 | 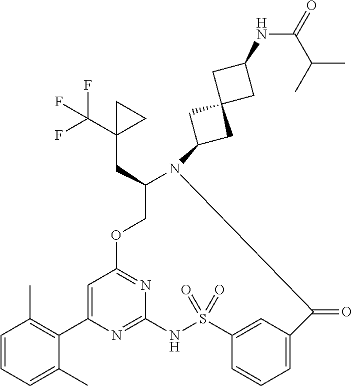 | | | +++ | +++ |
| 171 | 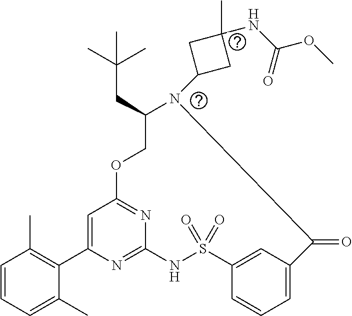 [SMILES: COC(=O)NC1(C)CC(N2C(=O)c3cccc(c3)S(=O)(=O)Nc3nc(cc(-c4c(C)cccc4C)n3)OC[C@H]2CC(C)(C)C)C1] | | | +++ | +++ |
| 172 | 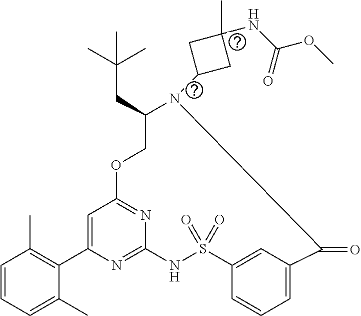 [SMILES: COC(=O)NC1(C)CC(N2C(=O)c3cccc(c3)S(=O)(=O)Nc3nc(cc(-c4c(C)cccc4C)n3)OC[C@H]2CC(C)(C)C)C1] | | | +++ | +++ |
| 124 | 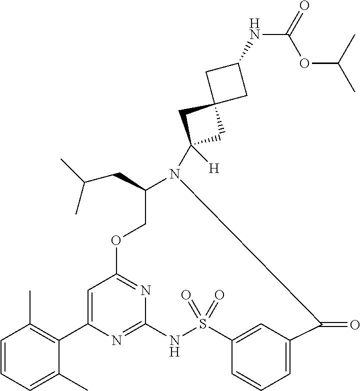 | | | +++ | +++ |
| 245 | 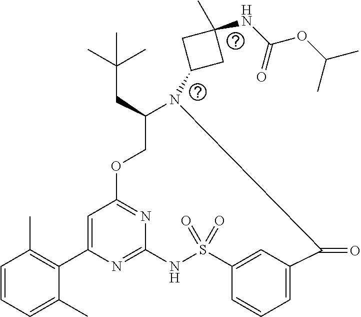 [SMILES: Cc1cccc(C)c1-c1cc2nc(n1)NS(=O)(=O)c1cccc(c1)C(=O)N([C@H]1C[C@](C)(NC(=O)OC(C)C)C1)[C@H](CC(C)(C)C)CO2] | | | +++ | +++ |
| 244 | 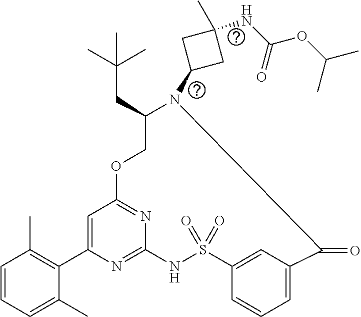 [SMILES: Cc1cccc(C)c1-c1cc2nc(n1)NS(=O)(=O)c1cccc(c1)C(=O)N([C@H]1C[C@](C)(NC(=O)OC(C)C)C1)[C@H](CC(C)(C)C)CO2] | | | +++ | ++ |
| 243 | 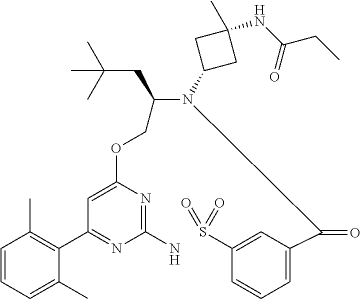 | | | +++ | +++ |
| 242 | 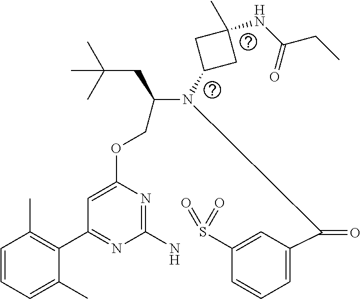 | | | +++ | ++ |
| 173 | 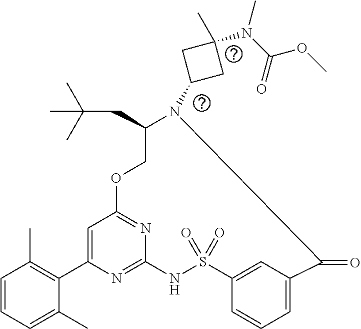 [SMILES: COC(=O)N(C)[C@]1(C)C[C@H](N2C(=O)c3cccc(c3)S(=O)(=O)Nc3nc(cc(-c4c(C)cccc4C)n3)OC[C@H]2CC(C)(C)C)C1] | | | +++ | ++ |
| 241 | 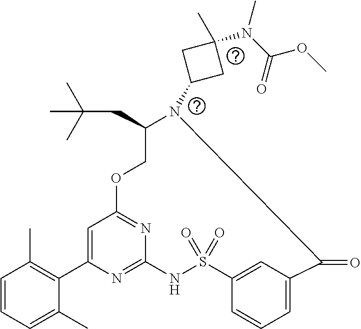 [SMILES: COC(=O)N(C)[C@]1(C)C[C@H](N2C(=O)c3cccc(c3)S(=O)(=O)Nc3nc(cc(-c4c(C)cccc4C)n3)OC[C@H]2CC(C)(C)C)C1] | | | +++ | ++ |
| 240 | 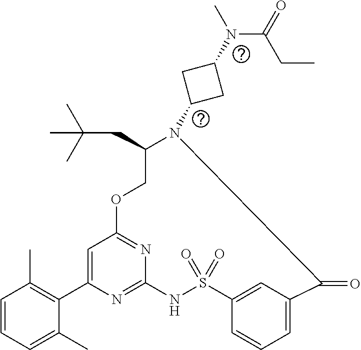 [SMILES: CCC(=O)N(C)[C@H]1C[C@@H](N2C(=O)c3cccc(c3)S(=O)(=O)Nc3nc(cc(-c4c(C)cccc4C)n3)OC[C@H]2CC(C)(C)C)C1] | | | +++ | ++ |
| 239 | 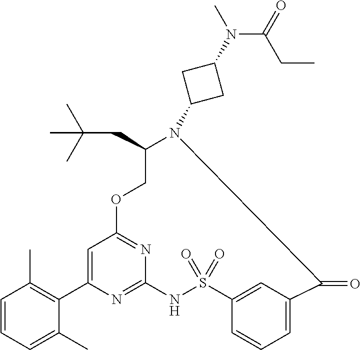 [SMILES: CCC(=O)N(C)[C@H]1C[C@@H](N2C(=O)c3cccc(c3)S(=O)(=O)Nc3nc(cc(-c4c(C)cccc4C)n3)OC[C@H]2CC(C)(C)C)C1] | | | +++ | ++ |
| 174 | 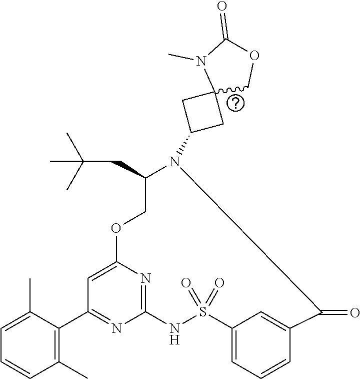 | | | +++ | ++ |
| 177 | 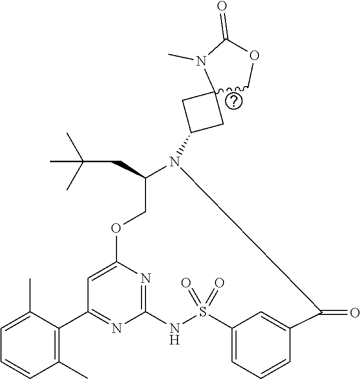 | | | +++ | ++ |
| 114 | 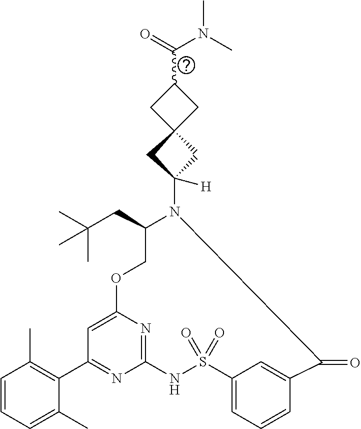 | | | +++ | +++ |
| 115 | 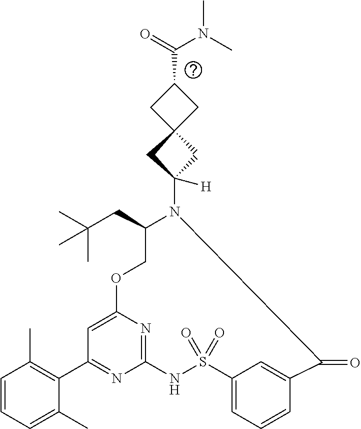 | | | +++ | +++ |
| 116 | 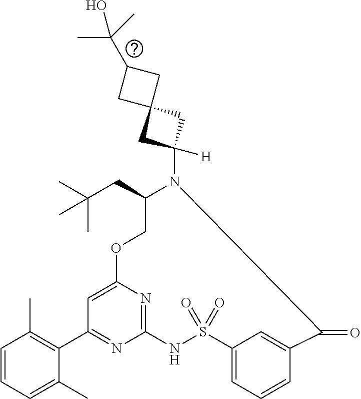 | | | +++ | +++ |
| 117 | 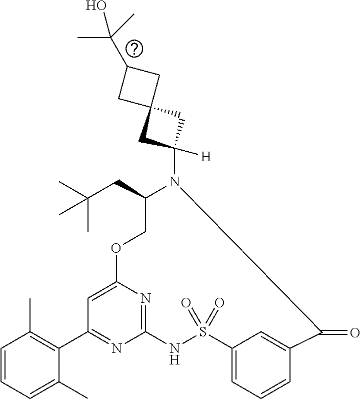 | | | +++ | +++ |
| 443 | 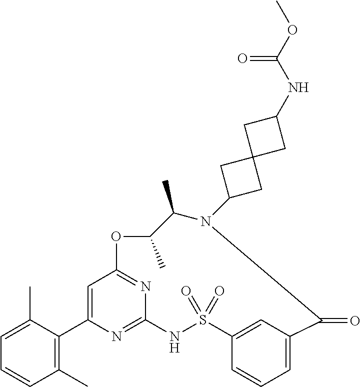 [SMILES: COC(=O)NC1CC2(C1)CC(N1C(=O)c3cccc(c3)S(=O)(=O)Nc3nc(cc(-c4c(C)cccc4C)n3)O[C@@H](C)[C@H]1C)C2] | | | +++ | ++ |
| 358 | 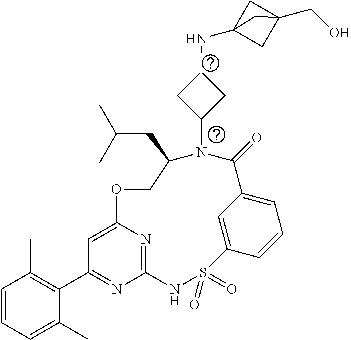 [SMILES: Cc1cccc(C)c1-c1cc2nc(n1)NS(=O)(=O)c1cccc(c1)C(=O)N(C1CC(NC34CC(CO)(C3)C4)C1)[C@H](CC(C)C)CO2] | | | +++ | +++ |
| 357 | 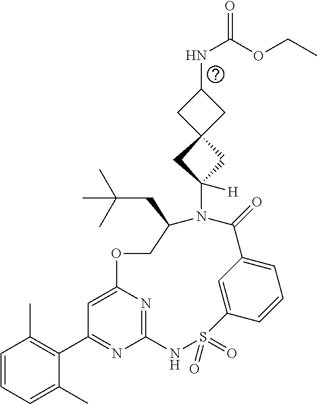 | | | +++ | +++ |
| 356 | 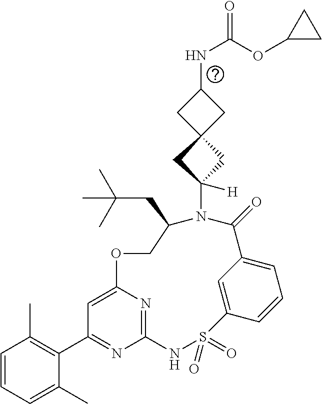 | | | +++ | +++ |
| 143 | 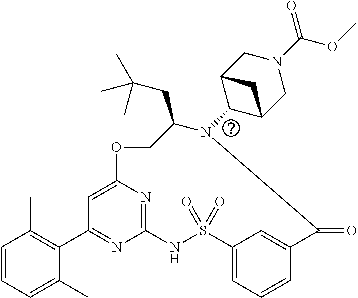 [SMILES: COC(=O)N1C[C@H]2C[C@@H](C1)[C@H]2N1C(=O)c2cccc(c2)S(=O)(=O)Nc2nc(cc(-c3c(C)cccc3C)n2)OC[C@H]1CC(C)(C)C] | | | +++ | ++ |
| 48 | 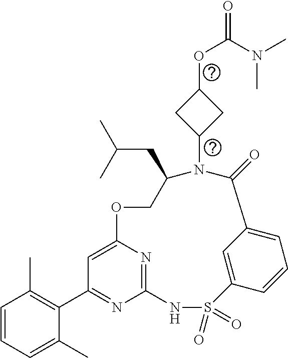 [SMILES: Cc1cccc(C)c1-c1cc2nc(n1)NS(=O)(=O)c1cccc(c1)C(=O)N(C1CC(OC(=O)N(C)C)C1)[C@H](CC(C)C)CO2] | | | +++ | ++ |
| 238 | 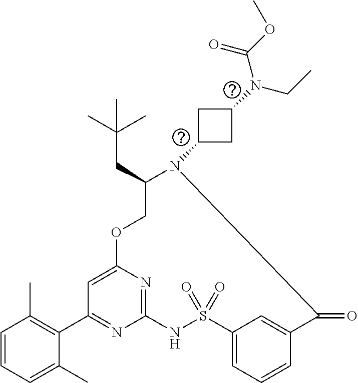 [SMILES: CCN(C(=O)OC)[C@H]1C[C@@H](N2C(=O)c3cccc(c3)S(=O)(=O)Nc3nc(cc(-c4c(C)cccc4C)n3)OC[C@H]2CC(C)(C)C)C1] | | | +++ | ++ |
| 237 | 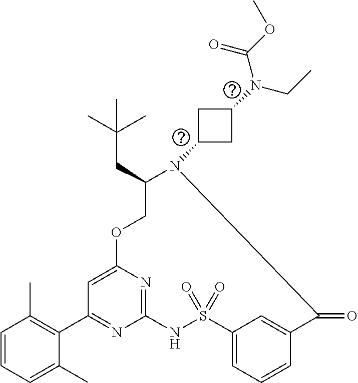 [SMILES: CCN(C(=O)OC)[C@H]1C[C@@H](N2C(=O)c3cccc(c3)S(=O)(=O)Nc3nc(cc(-c4c(C)cccc4C)n3)OC[C@H]2CC(C)(C)C)C1] | | | +++ | ++ |
| 236 | 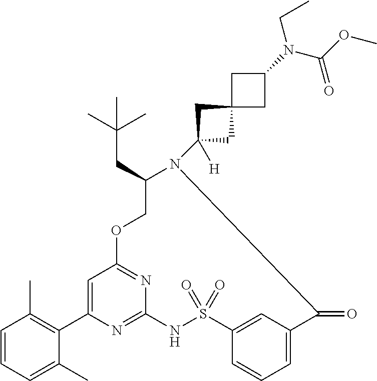 | | | +++ | ++ |
| 119 | 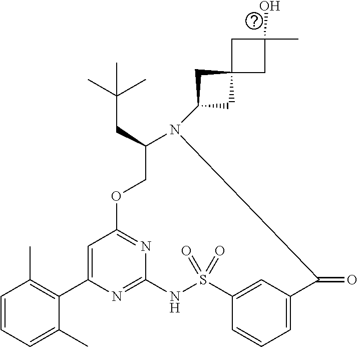 | | | +++ | +++ |
| 120 | 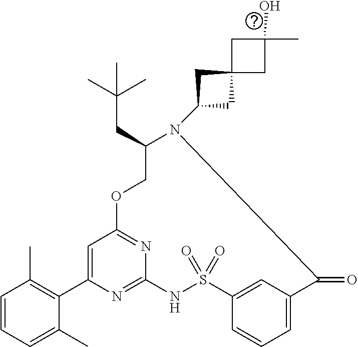 | | | +++ | ++ |
| 355 | 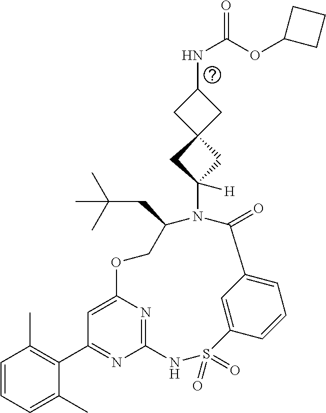 | | | +++ | ++ |
| 354 | 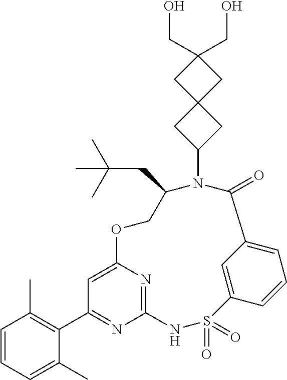 [SMILES: Cc1cccc(C)c1-c1cc2nc(n1)NS(=O)(=O)c1cccc(c1)C(=O)N(C1CC3(C1)CC(CO)(CO)C3)[C@H](CC(C)(C)C)CO2] | | | +++ | +++ |
| 94 | 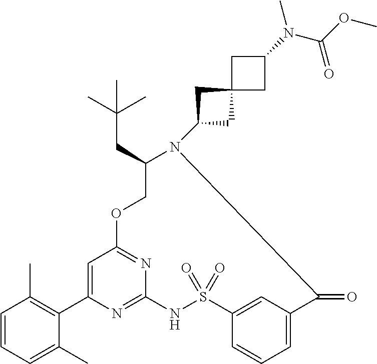 | | | +++ | ++ |
| 158 | 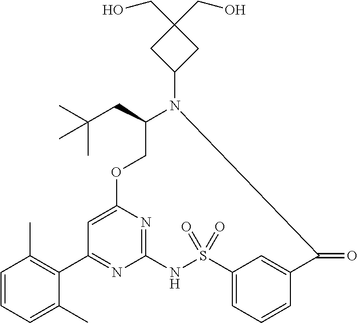 [SMILES: Cc1cccc(C)c1-c1cc2nc(n1)NS(=O)(=O)c1cccc(c1)C(=O)N(C1CC(CO)(CO)C1)[C@H](CC(C)(C)C)CO2] | | | ++ | +++ |
| 159 | 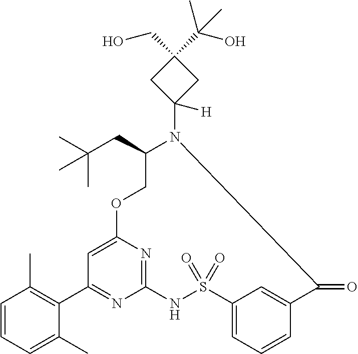 | | | +++ | +++ |
| 160 | 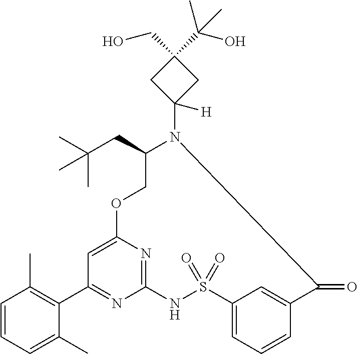 | | | +++ | +++ |
| 353 | 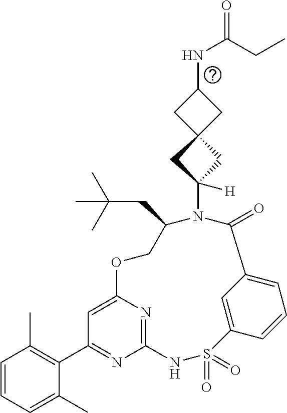 | | | +++ | +++ |
| 121 | 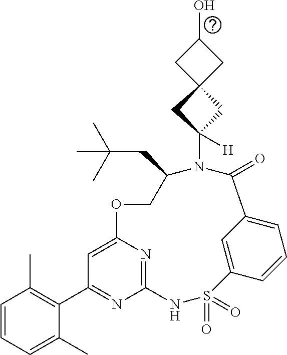 | | | +++ | +++ |
| 122 | 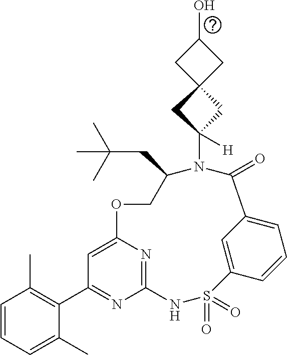 | | | +++ | +++ |
| 235 | 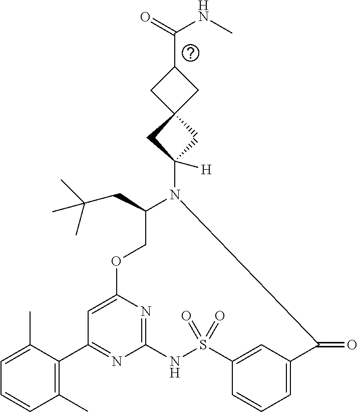 | | | +++ | +++ |
| 234 | 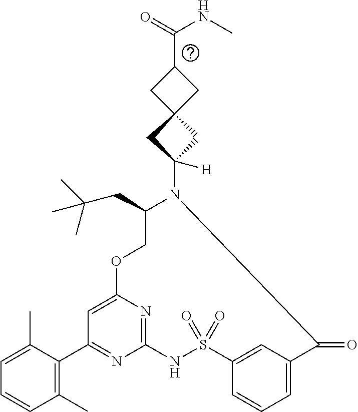 | | | +++ | +++ |
| 233 | 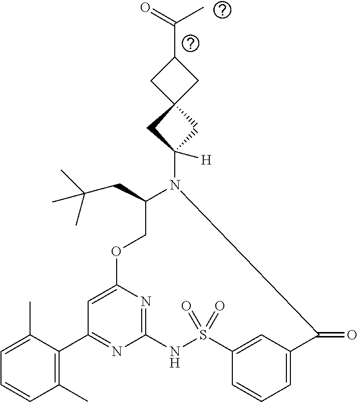 | | | +++ | +++ |
| 232 | 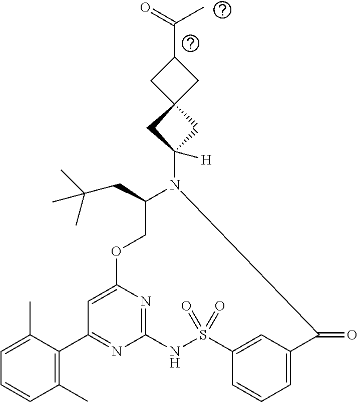 | | | +++ | +++ |
| 141 | 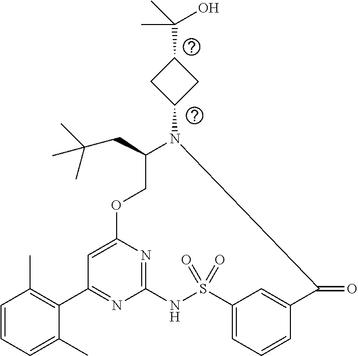 [SMILES: Cc1cccc(C)c1-c1cc2nc(n1)NS(=O)(=O)c1cccc(c1)C(=O)N([C@H]1C[C@@H](C(C)(C)O)C1)[C@H](CC(C)(C)C)CO2] | | | +++ | +++ |
| 142 | 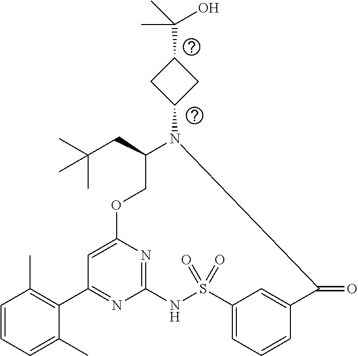 [SMILES: Cc1cccc(C)c1-c1cc2nc(n1)NS(=O)(=O)c1cccc(c1)C(=O)N([C@H]1C[C@@H](C(C)(C)O)C1)[C@H](CC(C)(C)C)CO2] | | | +++ | +++ |
| 95 | 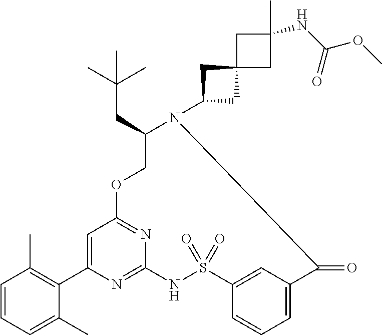 | | | +++ | +++ |
| 96 | 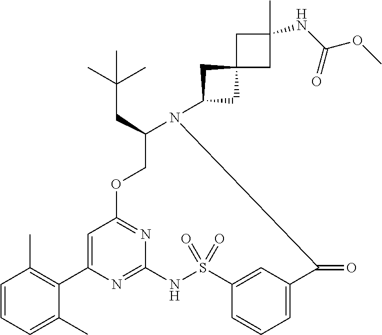 | | | +++ | ++ |
| 231 | 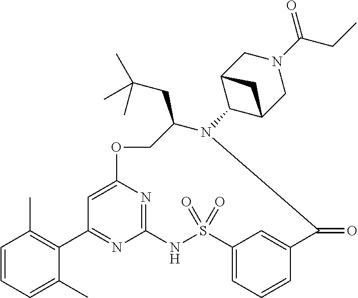 [SMILES: CCC(=O)N1C[C@H]2C[C@@H](C1)[C@H]2N1C(=O)c2cccc(c2)S(=O)(=O)Nc2nc(cc(-c3c(C)cccc3C)n2)OC[C@H]1CC(C)(C)C] | | | +++ | ++ |
| 161 | 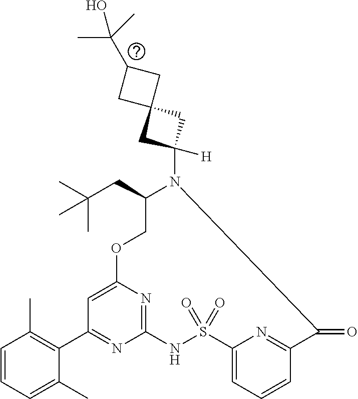 | | | +++ | +++ |
| 162 | 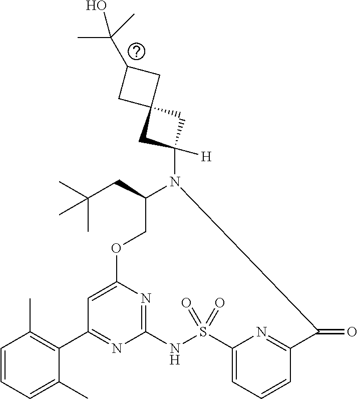 | | | +++ | +++ |
| 77 | 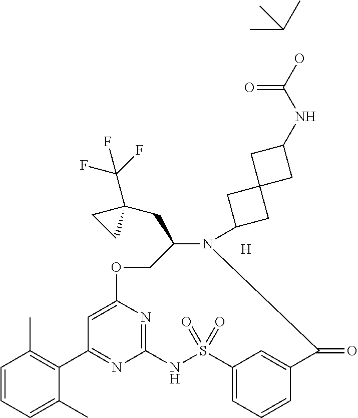 | | | | |
| 352 | 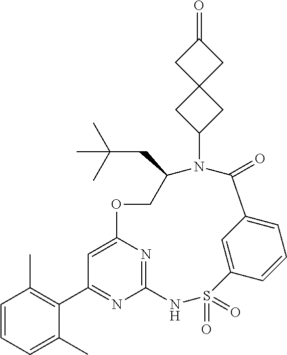 [SMILES: Cc1cccc(C)c1-c1cc2nc(n1)NS(=O)(=O)c1cccc(c1)C(=O)N(C1CC3(CC(=O)C3)C1)[C@H](CC(C)(C)C)CO2] | | | +++ | +++ |
| 415 | 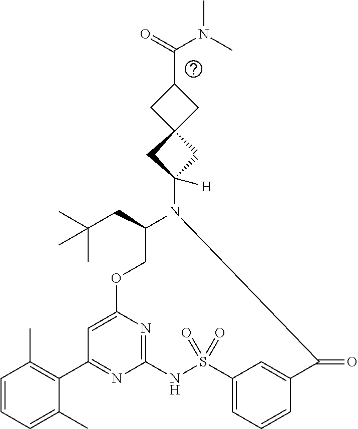 | | | +++ | +++ |
| 414 | 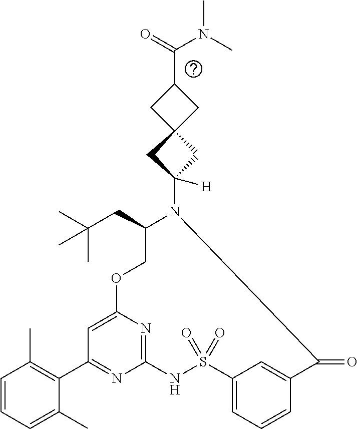 | | | +++ | +++ |
| 211 | 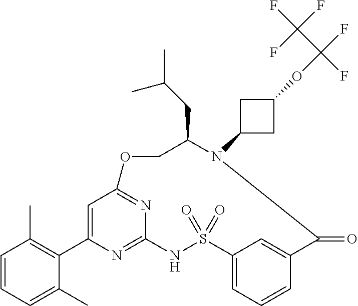 [SMILES: Cc1cccc(C)c1-c1cc2nc(n1)NS(=O)(=O)c1cccc(c1)C(=O)N([C@H]1C[C@H](OC(F)(F)C(F)(F)F)C1)[C@H](CC(C)C)CO2] | | | ND | + |
| 2 | 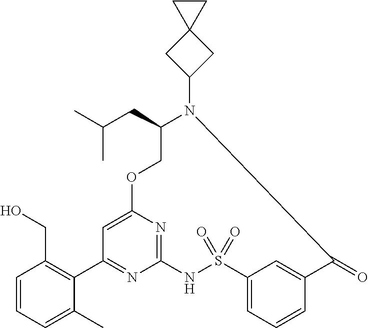 [SMILES: Cc1cccc(CO)c1-c1cc2nc(n1)NS(=O)(=O)c1cccc(c1)C(=O)N(C1CC3(CC3)C1)[C@H](CC(C)C)CO2] | | | +++ | ++ |
| 49 | 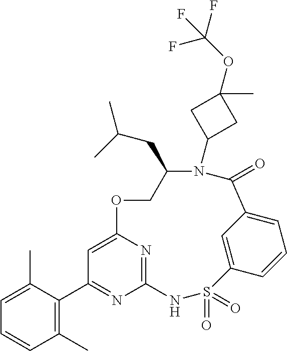 [SMILES: Cc1cccc(C)c1-c1cc2nc(n1)NS(=O)(=O)c1cccc(c1)C(=O)N(C1CC(C)(OC(F)(F)F)C1)[C@H](CC(C)C)CO2] | | | +++ | ++ |
| 50 | 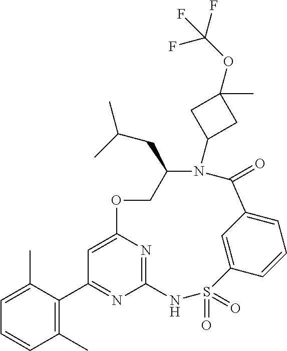 [SMILES: Cc1cccc(C)c1-c1cc2nc(n1)NS(=O)(=O)c1cccc(c1)C(=O)N(C1CC(C)(OC(F)(F)F)C1)[C@H](CC(C)C)CO2] | | | +++ | ++ |
| 156 | 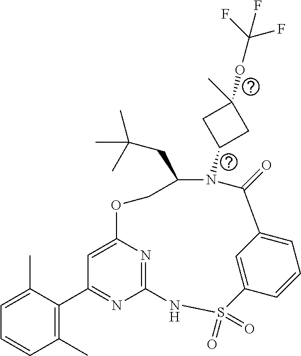 [SMILES: Cc1cccc(C)c1-c1cc2nc(n1)NS(=O)(=O)c1cccc(c1)C(=O)N([C@H]1C[C@@](C)(OC(F)(F)F)C1)[C@H](CC(C)(C)C)CO2] | | | +++ | ++ |
| 157 | 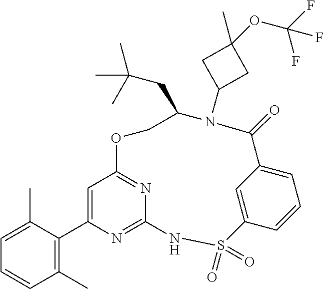 [SMILES: Cc1cccc(C)c1-c1cc2nc(n1)NS(=O)(=O)c1cccc(c1)C(=O)N(C1CC(C)(OC(F)(F)F)C1)[C@H](CC(C)(C)C)CO2] | | | +++ | ++ |
| 210 | 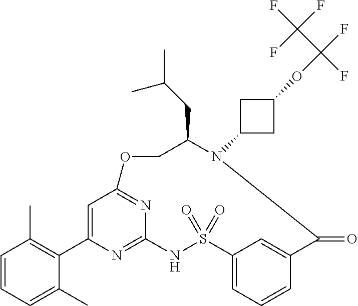 [SMILES: Cc1cccc(C)c1-c1cc2nc(n1)NS(=O)(=O)c1cccc(c1)C(=O)N([C@H]1C[C@@H](OC(F)(F)C(F)(F)F)C1)[C@H](CC(C)C)CO2] | | | +++ | ++ |
| 412 | 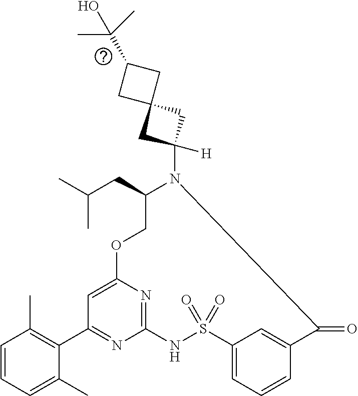 | | | +++ | +++ |
| 413 | 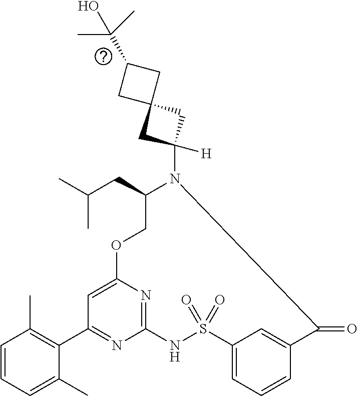 | | | +++ | +++ |
| 410 | 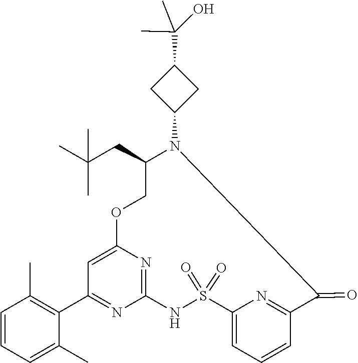 [SMILES: Cc1cccc(C)c1-c1cc2nc(n1)NS(=O)(=O)c1cccc(n1)C(=O)N([C@H]1C[C@@H](C(C)(C)O)C1)[C@H](CC(C)(C)C)CO2] | | | +++ | +++ |
| 411 | 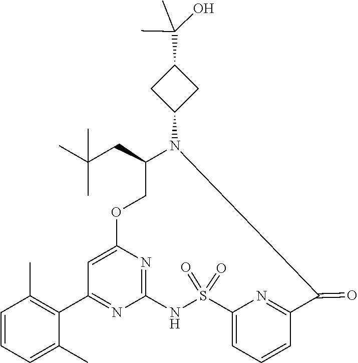 [SMILES: Cc1cccc(C)c1-c1cc2nc(n1)NS(=O)(=O)c1cccc(n1)C(=O)N([C@H]1C[C@@H](C(C)(C)O)C1)[C@H](CC(C)(C)C)CO2] | | | +++ | +++ |
| 52 | 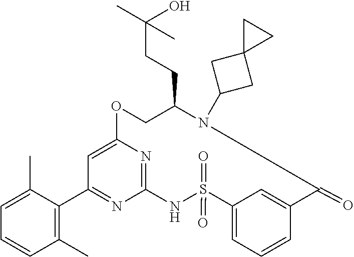 [SMILES: Cc1cccc(C)c1-c1cc2nc(n1)NS(=O)(=O)c1cccc(c1)C(=O)N(C1CC3(CC3)C1)[C@H](CCC(C)(C)O)CO2] | | | +++ | ++ |
| 152 | 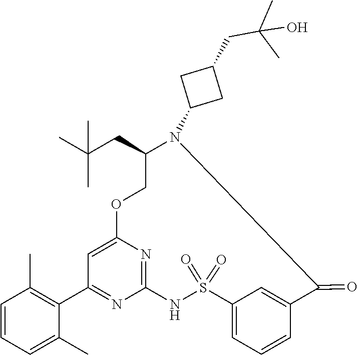 [SMILES: Cc1cccc(C)c1-c1cc2nc(n1)NS(=O)(=O)c1cccc(c1)C(=O)N([C@H]1C[C@@H](CC(C)(C)O)C1)[C@H](CC(C)(C)C)CO2] | | | +++ | +++ |
| 153 | 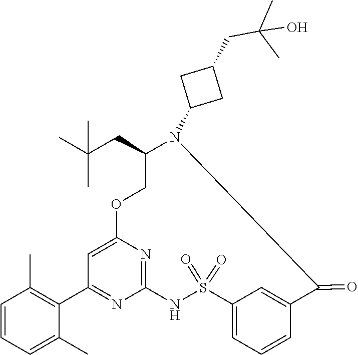 [SMILES: Cc1cccc(C)c1-c1cc2nc(n1)NS(=O)(=O)c1cccc(c1)C(=O)N([C@H]1C[C@@H](CC(C)(C)O)C1)[C@H](CC(C)(C)C)CO2] | | | +++ | +++ |
| 51 | 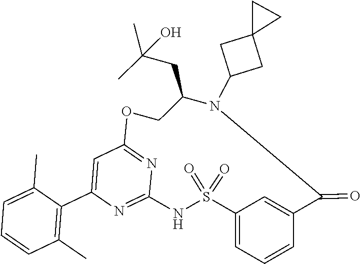 [SMILES: Cc1cccc(C)c1-c1cc2nc(n1)NS(=O)(=O)c1cccc(c1)C(=O)N(C1CC3(CC3)C1)[C@H](CC(C)(C)O)CO2] | | | ++ | +++ |
| 408 | 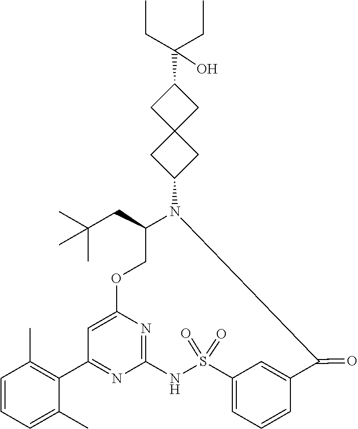 [SMILES: CCC(O)(CC)[C@H]1CC2(C[C@H](N3C(=O)c4cccc(c4)S(=O)(=O)Nc4nc(cc(-c5c(C)cccc5C)n4)OC[C@H]3CC(C)(C)C)C2)C1] | | | +++ | +++ |
| 409 | 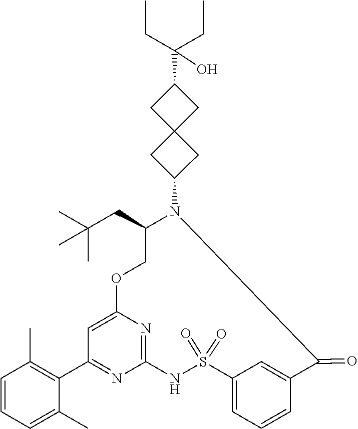 [SMILES: CCC(O)(CC)[C@H]1CC2(C[C@H](N3C(=O)c4cccc(c4)S(=O)(=O)Nc4nc(cc(-c5c(C)cccc5C)n4)OC[C@H]3CC(C)(C)C)C2)C1] | | | +++ | +++ |
| 97 | 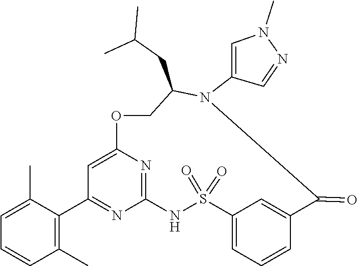 [SMILES: Cc1cccc(C)c1-c1cc2nc(n1)NS(=O)(=O)c1cccc(c1)C(=O)N(c1cnn(C)c1)[C@H](CC(C)C)CO2] | | | +++ | +++ |
| 230 | 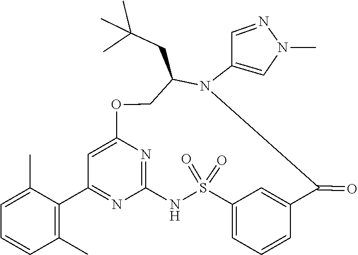 [SMILES: Cc1cccc(C)c1-c1cc2nc(n1)NS(=O)(=O)c1cccc(c1)C(=O)N(c1cnn(C)c1)[C@H](CC(C)(C)C)CO2] | | | +++ | +++ |
| 147 | 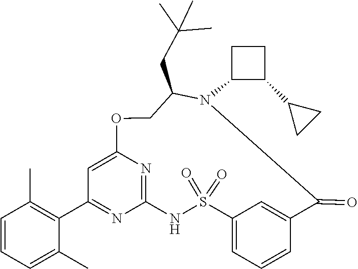 [SMILES: Cc1cccc(C)c1-c1cc2nc(n1)NS(=O)(=O)c1cccc(c1)C(=O)N([C@@H]1CC[C@@H]1C1CC1)[C@H](CC(C)(C)C)CO2] | | | +++ | ++ |
| 148 | 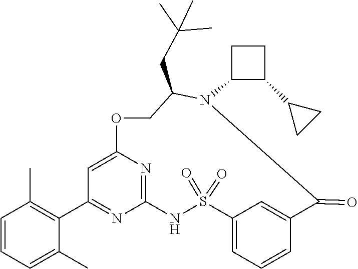 [SMILES: Cc1cccc(C)c1-c1cc2nc(n1)NS(=O)(=O)c1cccc(c1)C(=O)N([C@@H]1CC[C@@H]1C1CC1)[C@H](CC(C)(C)C)CO2] | | | +++ | +++ |
| 149 | 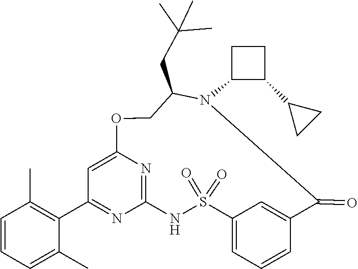 [SMILES: Cc1cccc(C)c1-c1cc2nc(n1)NS(=O)(=O)c1cccc(c1)C(=O)N([C@@H]1CC[C@@H]1C1CC1)[C@H](CC(C)(C)C)CO2] | | | +++ | +++ |
| 41 | 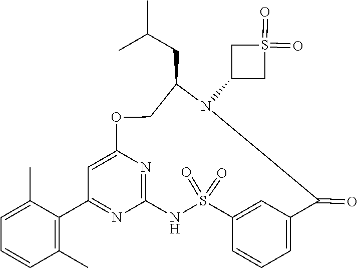 | | | +++ | +++ |
| 207 | 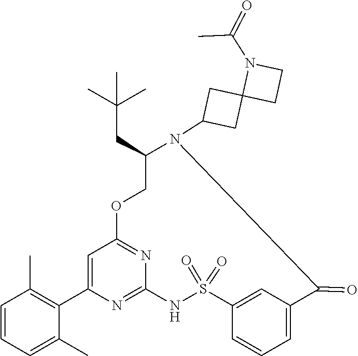 [SMILES: CC(=O)N1CCC12CC(N1C(=O)c3cccc(c3)S(=O)(=O)Nc3nc(cc(-c4c(C)cccc4C)n3)OC[C@H]1CC(C)(C)C)C2] | | | +++ | +++ |
| 404 | 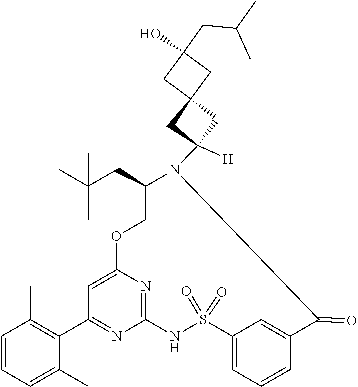 | | | +++ | +++ |
| 405 | 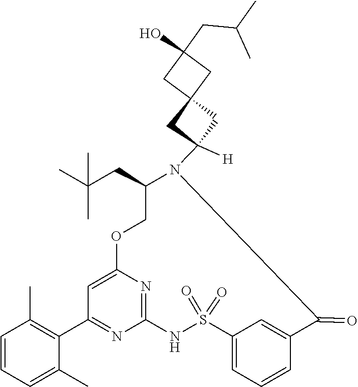 | | | +++ | +++ |
| 4 | 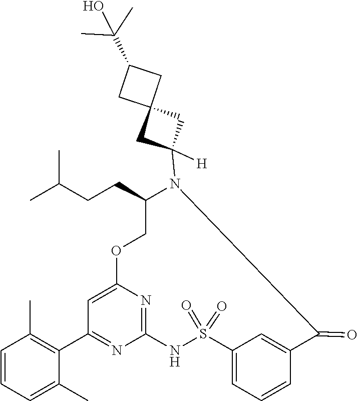 | | | +++ | +++ |
| 5 | 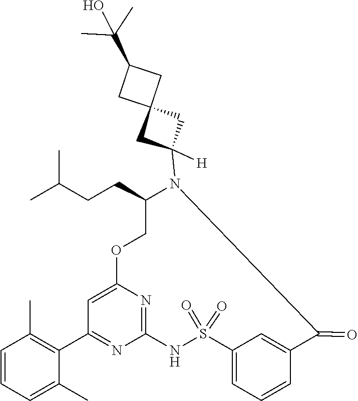 | | | +++ | +++ |
| 208 | 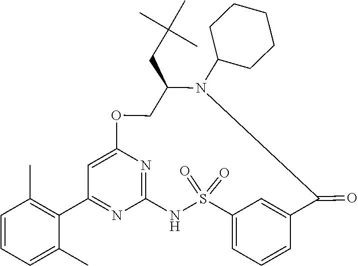 [SMILES: Cc1cccc(C)c1-c1cc2nc(n1)NS(=O)(=O)c1cccc(c1)C(=O)N(C1CCCCC1)[C@H](CC(C)(C)C)CO2] | | | ND | + |
| 228 | 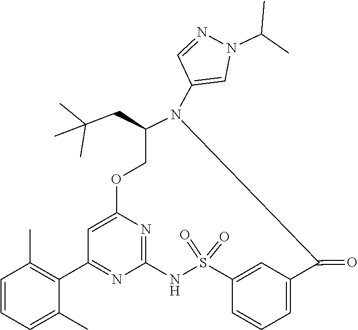 [SMILES: Cc1cccc(C)c1-c1cc2nc(n1)NS(=O)(=O)c1cccc(c1)C(=O)N(c1cnn(C(C)C)c1)[C@H](CC(C)(C)C)CO2] | | | +++ | +++ |
| 229 | 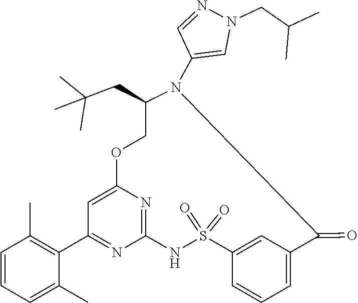 [SMILES: Cc1cccc(C)c1-c1cc2nc(n1)NS(=O)(=O)c1cccc(c1)C(=O)N(c1cnn(CC(C)C)c1)[C@H](CC(C)(C)C)CO2] | | | +++ | +++ |
| 146 | 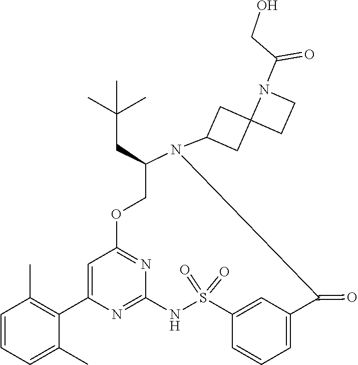 [SMILES: Cc1cccc(C)c1-c1cc2nc(n1)NS(=O)(=O)c1cccc(c1)C(=O)N(C1CC3(CCN3C(=O)CO)C1)[C@H](CC(C)(C)C)CO2] | | | +++ | +++ |
| 7 | 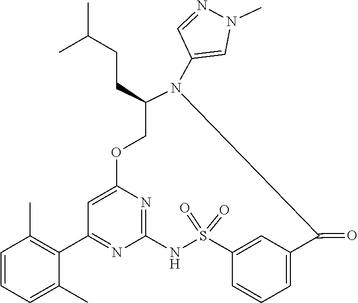 [SMILES: Cc1cccc(C)c1-c1cc2nc(n1)NS(=O)(=O)c1cccc(c1)C(=O)N(c1cnn(C)c1)[C@H](CCC(C)C)CO2] | | | +++ | +++ |
| 227 | 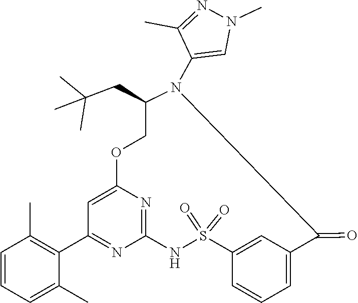 [SMILES: Cc1cccc(C)c1-c1cc2nc(n1)NS(=O)(=O)c1cccc(c1)C(=O)N(c1cn(C)nc1C)[C@H](CC(C)(C)C)CO2] | | | +++ | +++ |
| 435 | 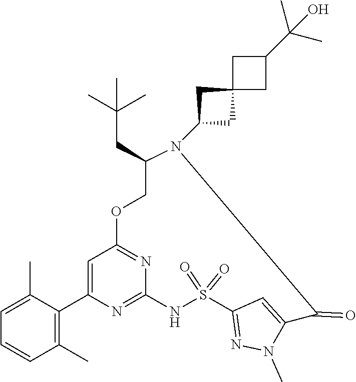 | | | | |
| 436 | 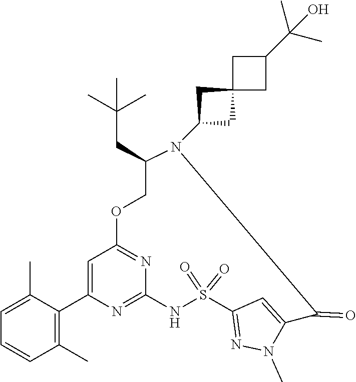 | | | +++ | +++ |
| 226 | 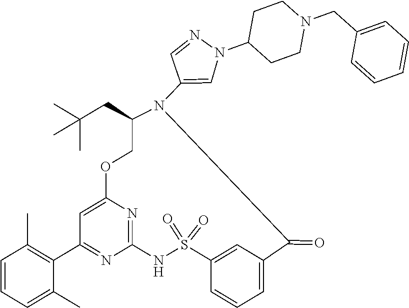 [SMILES: Cc1cccc(C)c1-c1cc2nc(n1)NS(=O)(=O)c1cccc(c1)C(=O)N(c1cnn(C3CCN(Cc4ccccc4)CC3)c1)[C@H](CC(C)(C)C)CO2] | | | +++ | + |
| 184 | 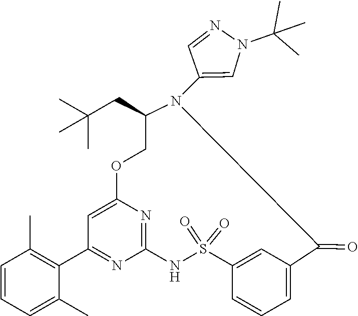 [SMILES: Cc1cccc(C)c1-c1cc2nc(n1)NS(=O)(=O)c1cccc(c1)C(=O)N(c1cnn(C(C)(C)C)c1)[C@H](CC(C)(C)C)CO2] | | | +++ | +++ |
| 154 | 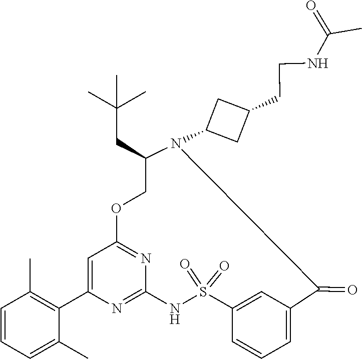 [SMILES: CC(=O)NCC[C@H]1C[C@@H](N2C(=O)c3cccc(c3)S(=O)(=O)Nc3nc(cc(-c4c(C)cccc4C)n3)OC[C@H]2CC(C)(C)C)C1] | | | +++ | +++ |
| 155 | 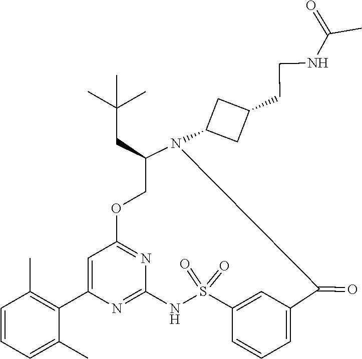 [SMILES: CC(=O)NCC[C@H]1C[C@@H](N2C(=O)c3cccc(c3)S(=O)(=O)Nc3nc(cc(-c4c(C)cccc4C)n3)OC[C@H]2CC(C)(C)C)C1] | | | +++ | +++ |
| 98 | 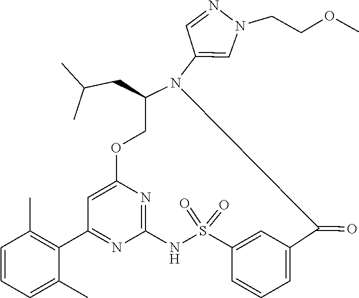 [SMILES: COCCn1cc(N2C(=O)c3cccc(c3)S(=O)(=O)Nc3nc(cc(-c4c(C)cccc4C)n3)OC[C@H]2CC(C)C)cn1] | | | +++ | +++ |
| 225 | 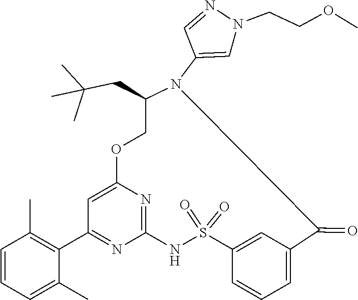 [SMILES: COCCn1cc(N2C(=O)c3cccc(c3)S(=O)(=O)Nc3nc(cc(-c4c(C)cccc4C)n3)OC[C@H]2CC(C)(C)C)cn1] | | | +++ | +++ |
| 6 | 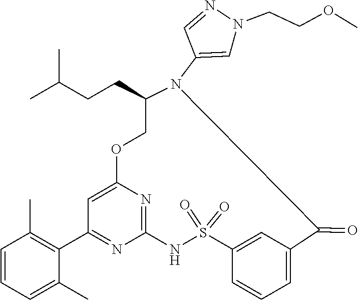 [SMILES: COCCn1cc(N2C(=O)c3cccc(c3)S(=O)(=O)Nc3nc(cc(-c4c(C)cccc4C)n3)OC[C@H]2CCC(C)C)cn1] | | | +++ | +++ |
| 224 | 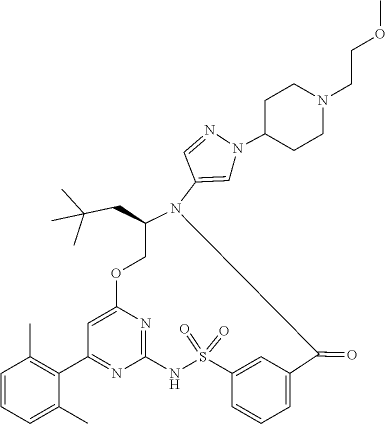 [SMILES: COCCN1CCC(n2cc(N3C(=O)c4cccc(c4)S(=O)(=O)Nc4nc(cc(-c5c(C)cccc5C)n4)OC[C@H]3CC(C)(C)C)cn2)CC1] | | | ND | + |
| 223 | 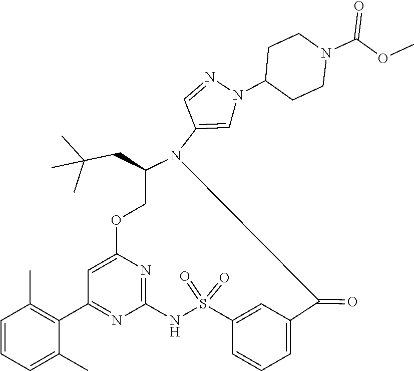 [SMILES: COC(=O)N1CCC(n2cc(N3C(=O)c4cccc(c4)S(=O)(=O)Nc4nc(cc(-c5c(C)cccc5C)n4)OC[C@H]3CC(C)(C)C)cn2)CC1] | | | +++ | + |
| 89 | 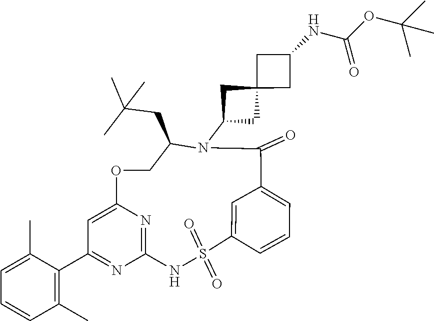 | | | | |
| 90 | 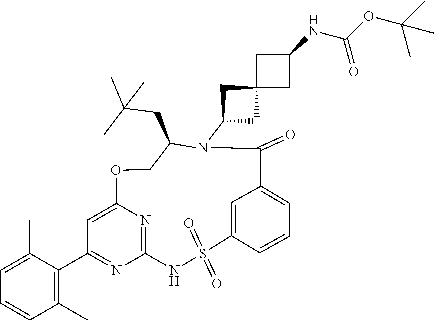 | | | | |
| 222 | 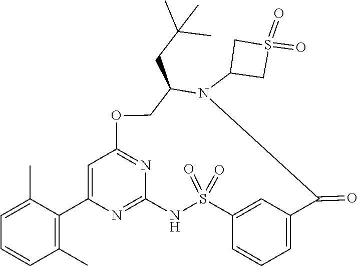 [SMILES: Cc1cccc(C)c1-c1cc2nc(n1)NS(=O)(=O)c1cccc(c1)C(=O)N(C1CS(=O)(=O)C1)[C@H](CC(C)(C)C)CO2] | | | +++ | +++ |
| 183 | 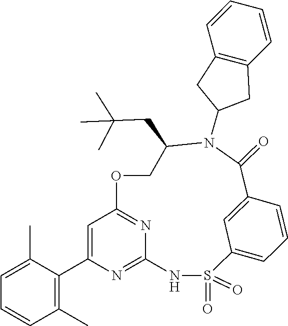 [SMILES: Cc1cccc(C)c1-c1cc2nc(n1)NS(=O)(=O)c1cccc(c1)C(=O)N(C1Cc3ccccc3C1)[C@H](CC(C)(C)C)CO2] | | | +++ | +++ |
| 8 | 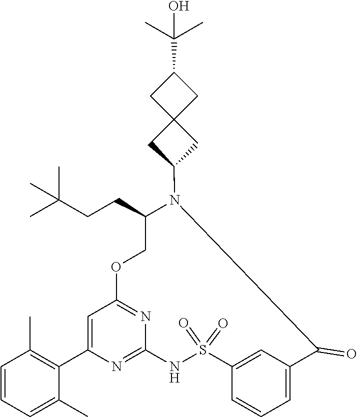 | | | +++ | +++ |
| 9 | 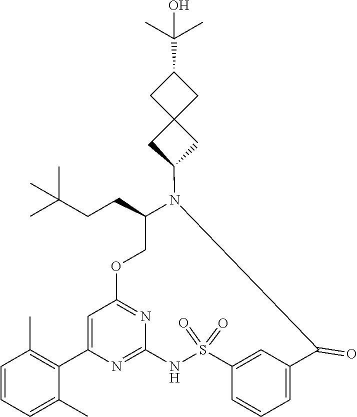 | | | +++ | +++ |

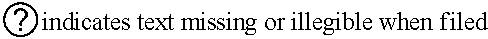

Table 11 represents CFTR modulating activity for representative compounds of the disclosure generated using one or more of the assays described in this example (EC50: +++ is <1 μM; ++ is 1-<3 μM; + is 3-<30 μM; and ND is “not detected in this assay.” 0% Activity: +++ is >60%; ++ is 30-60%; + is <300%).

TABLE 11
Biological Activity Data

| | | | | HBE |
|---|---|---|---|---|
| | | HBE | HBE Max | Activity at |
| | | EC₅₀ | Activity | 10 μM |
| Cmpd No. | Structure | (μM) | (%) | (%) |
| 220 | 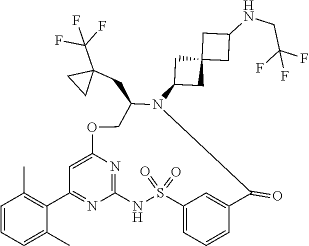 | +++ | +++ | |
| 221 | 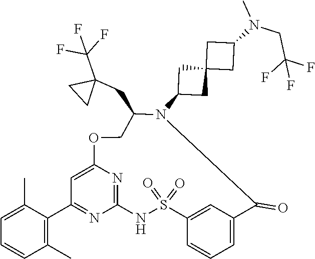 | +++ | +++ | |
| 206 | 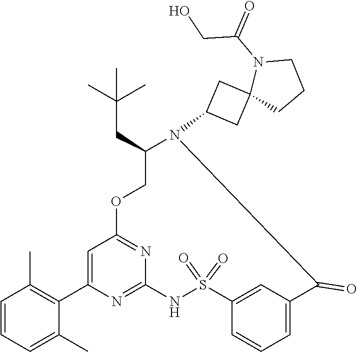 [SMILES: Cc1cccc(C)c1-c1cc2nc(n1)NS(=O)(=O)c1cccc(c1)C(=O)N([C@H]1C[C@@]3(CCCN3C(=O)CO)C1)[C@H](CC(C)(C)C)CO2] | +++ | +++ | |
| 205 | 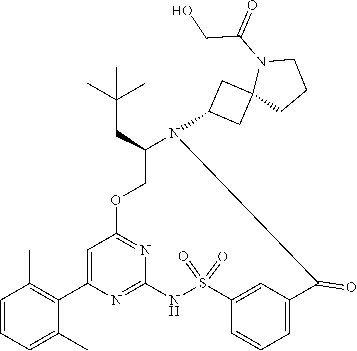 [SMILES: Cc1cccc(C)c1-c1cc2nc(n1)NS(=O)(=O)c1cccc(c1)C(=O)N([C@H]1C[C@@]3(CCCN3C(=O)CO)C1)[C@H](CC(C)(C)C)CO2] | +++ | +++ | |
| 204 | 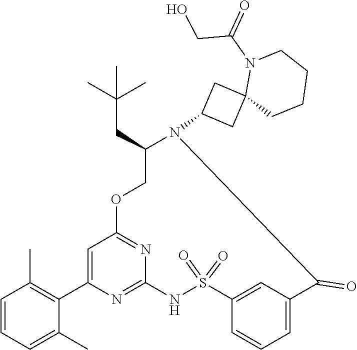 [SMILES: Cc1cccc(C)c1-c1cc2nc(n1)NS(=O)(=O)c1cccc(c1)C(=O)N([C@H]1C[C@@]3(CCCCN3C(=O)CO)C1)[C@H](CC(C)(C)C)CO2] | | | +++ |
| 203 | 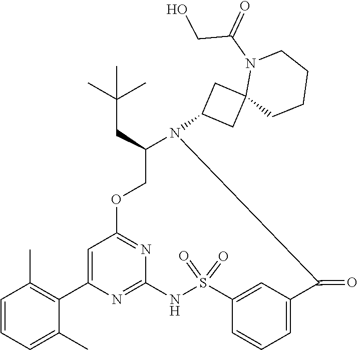 [SMILES: Cc1cccc(C)c1-c1cc2nc(n1)NS(=O)(=O)c1cccc(c1)C(=O)N([C@H]1C[C@@]3(CCCCN3C(=O)CO)C1)[C@H](CC(C)(C)C)CO2] | | | +++ |
| 144 | 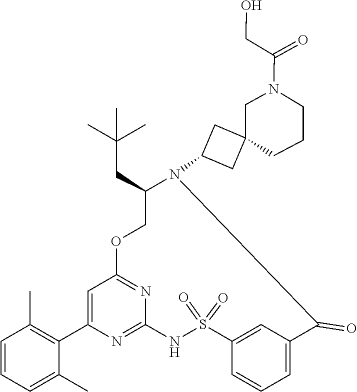 [SMILES: Cc1cccc(C)c1-c1cc2nc(n1)NS(=O)(=O)c1cccc(c1)C(=O)N([C@H]1C[C@@]3(CCCN(C(=O)CO)C3)C1)[C@H](CC(C)(C)C)CO2] | +++ | +++ | |
| 145 | 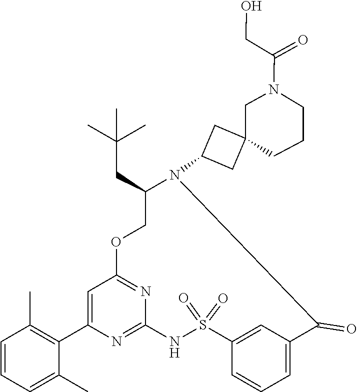 [SMILES: Cc1cccc(C)c1-c1cc2nc(n1)NS(=O)(=O)c1cccc(c1)C(=O)N([C@H]1C[C@@]3(CCCN(C(=O)CO)C3)C1)[C@H](CC(C)(C)C)CO2] | +++ | +++ | |
| 218 | 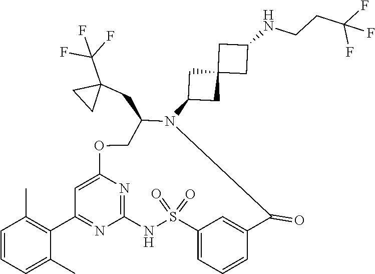 | +++ | +++ | |
| 219 | 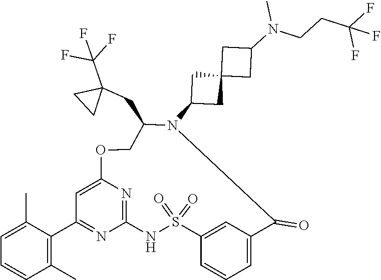 | | | +++ |
| 99 | 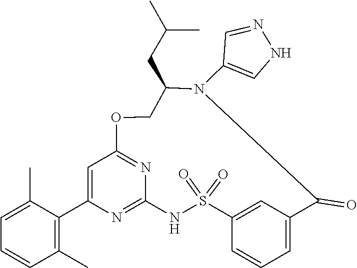 [SMILES: Cc1cccc(C)c1-c1cc2nc(n1)NS(=O)(=O)c1cccc(c1)C(=O)N(c1cn[nH]c1)[C@H](CC(C)C)CO2] | + | +++ | |
| 178 | 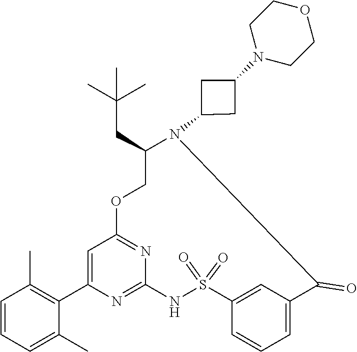 [SMILES: Cc1cccc(C)c1-c1cc2nc(n1)NS(=O)(=O)c1cccc(c1)C(=O)N([C@H]1C[C@@H](N3CCOCC3)C1)[C@H](CC(C)(C)C)CO2] | +++ | +++ | |
| 434 | 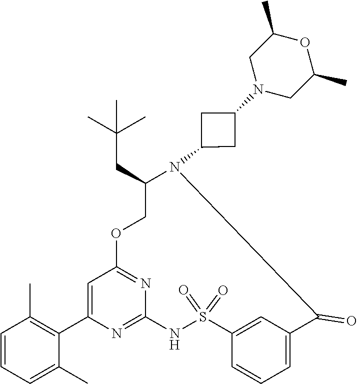 [SMILES: Cc1cccc(C)c1-c1cc2nc(n1)NS(=O)(=O)c1cccc(c1)C(=O)N([C@H]1C[C@@H](N3C[C@@H](C)O[C@@H](C)C3)C1)[C@H](CC(C)(C)C)CO2] | | | +++ |
| 140 | 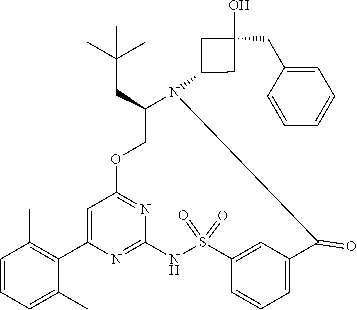 [SMILES: Cc1cccc(C)c1-c1cc2nc(n1)NS(=O)(=O)c1cccc(c1)C(=O)N([C@H]1C[C@@](O)(Cc3ccccc3)C1)[C@H](CC(C)(C)C)CO2] | +++ | +++ | |
| 139 | 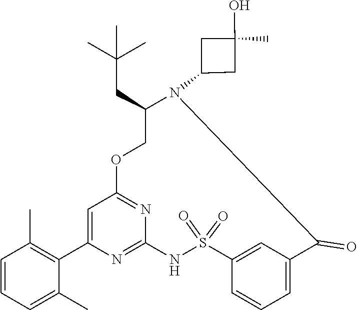 [SMILES: Cc1cccc(C)c1-c1cc2nc(n1)NS(=O)(=O)c1cccc(c1)C(=O)N([C@H]1C[C@](C)(O)C1)[C@H](CC(C)(C)C)CO2] | +++ | +++ | |
| 430 | 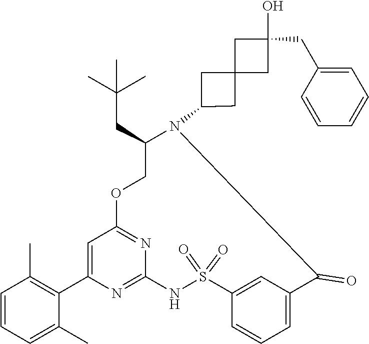 [SMILES: Cc1cccc(C)c1-c1cc2nc(n1)NS(=O)(=O)c1cccc(c1)C(=O)N([C@H]1CC3(C1)C[C@@](O)(Cc1ccccc1)C3)[C@H](CC(C)(C)C)CO2] | +++ | +++ | |
| 431 | 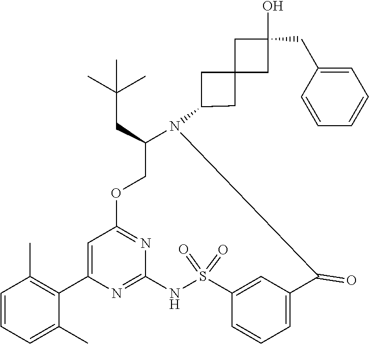 [SMILES: Cc1cccc(C)c1-c1cc2nc(n1)NS(=O)(=O)c1cccc(c1)C(=O)N([C@H]1CC3(C1)C[C@@](O)(Cc1ccccc1)C3)[C@H](CC(C)(C)C)CO2] | +++ | +++ | |
| 432 | 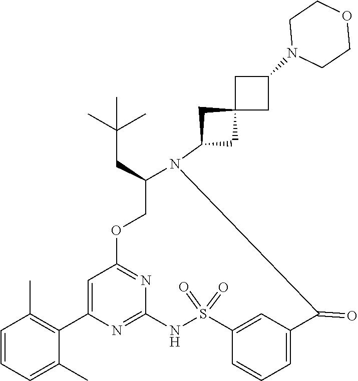 | +++ | +++ | |
| 433 | 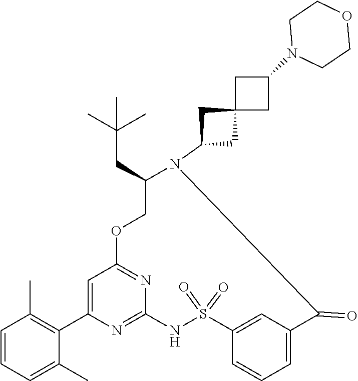 | +++ | +++ | |
| 87 | 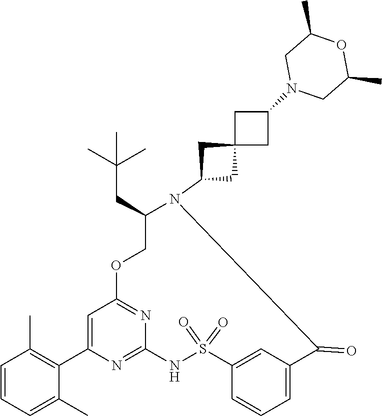 | +++ | +++ | |
| 86 | 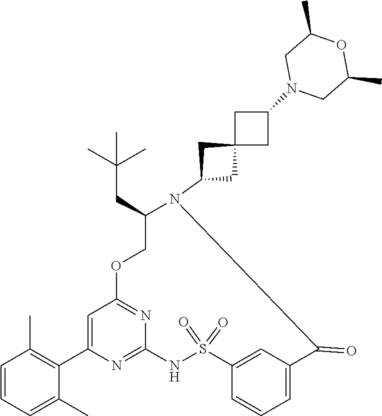 | +++ | +++ | |
| 185 | 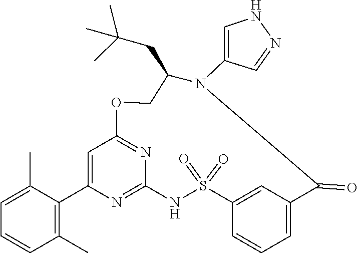 [SMILES: Cc1cccc(C)c1-c1cc2nc(n1)NS(=O)(=O)c1cccc(c1)C(=O)N(c1cn[nH]c1)[C@H](CC(C)(C)C)CO2] | +++ | +++ | |
| 350 | 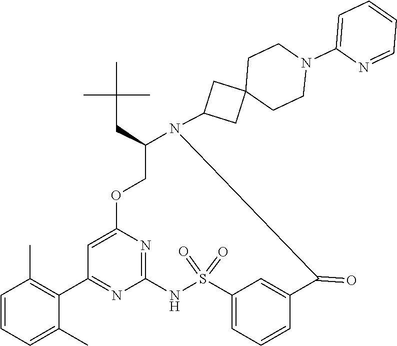 [SMILES: Cc1cccc(C)c1-c1cc2nc(n1)NS(=O)(=O)c1cccc(c1)C(=O)N(C1CC3(CCN(c4ccccn4)CC3)C1)[C@H](CC(C)(C)C)CO2] | +++ | +++ | |
| 209 | 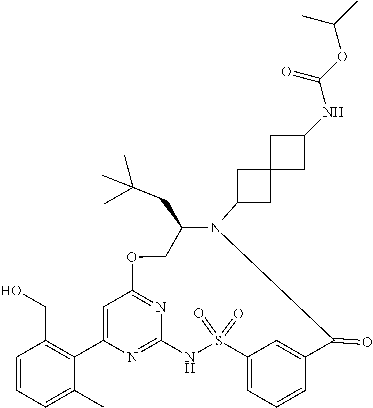 [SMILES: Cc1cccc(CO)c1-c1cc2nc(n1)NS(=O)(=O)c1cccc(c1)C(=O)N(C1CC3(CC(NC(=O)OC(C)C)C3)C1)[C@H](CC(C)(C)C)CO2] | | | ++ |
| 402 | 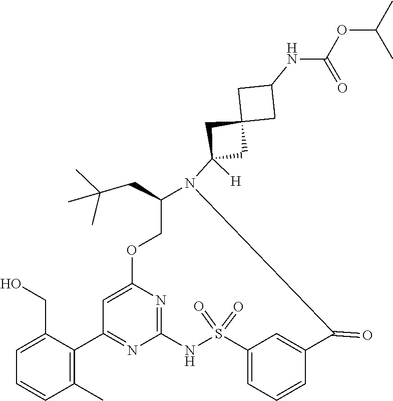 | | | ++ |
| 403 | 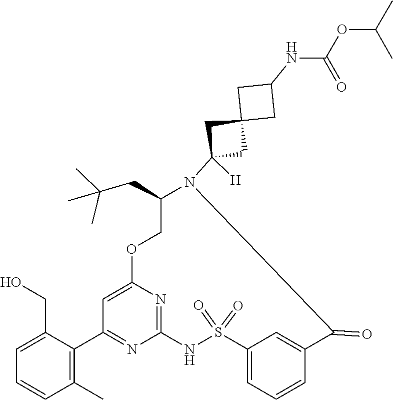 | | | ++ |
| 429 | 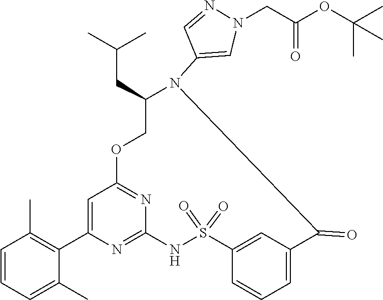 [SMILES: Cc1cccc(C)c1-c1cc2nc(n1)NS(=O)(=O)c1cccc(c1)C(=O)N(c1cnn(CC(=O)OC(C)(C)C)c1)[C@H](CC(C)C)CO2] | +++ | +++ | |
| 427 | 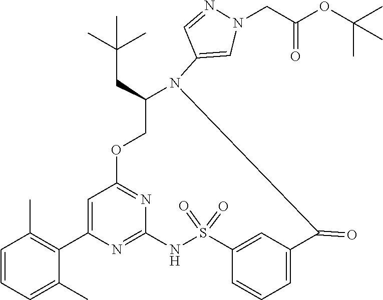 [SMILES: Cc1cccc(C)c1-c1cc2nc(n1)NS(=O)(=O)c1cccc(c1)C(=O)N(c1cnn(CC(=O)OC(C)(C)C)c1)[C@H](CC(C)(C)C)CO2] | +++ | +++ | |
| 426 | 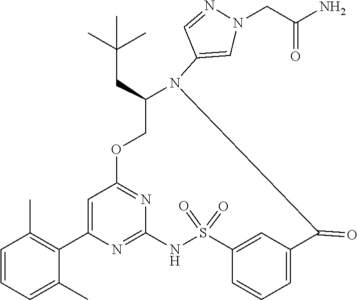 [SMILES: Cc1cccc(C)c1-c1cc2nc(n1)NS(=O)(=O)c1cccc(c1)C(=O)N(c1cnn(CC(N)=O)c1)[C@H](CC(C)(C)C)CO2] | ++ | +++ | |
| 428 | 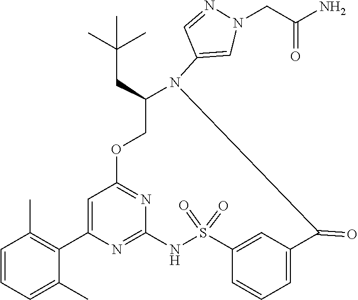 [SMILES: Cc1cccc(C)c1-c1cc2nc(n1)NS(=O)(=O)c1cccc(c1)C(=O)N(c1cnn(CC(N)=O)c1)[C@H](CC(C)(C)C)CO2] | | | + |
| 473 | 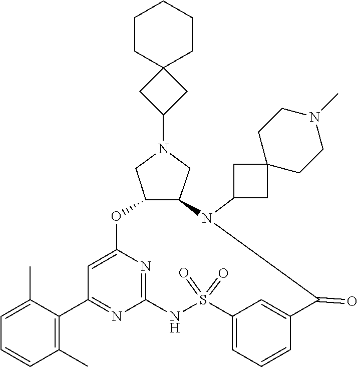 [SMILES: Cc1cccc(C)c1-c1cc2nc(n1)NS(=O)(=O)c1cccc(c1)C(=O)N(C1CC3(CCN(C)CC3)C1)[C@@H]1CN(C3CC4(CCCCC4)C3)C[C@H]1O2] | | | +++ |

### VIII. Synthesis of Compounds 507 and 508

### Example 158: Preparation of Compound 507

### Step 1: (11R)-6-(2,6-Dimethylphenyl)-11-(2,2-dimethylpropyl)-12-(1-methyl-2-oxo-3-piperidyl)-2,2-dioxo-9-oxa-2λ⁶-thia-3,5,12,19-tetrazatricyclo[12.3.1.14,8]nonadeca-1(18),4(19),5,7,14,16-hexaen-13-one

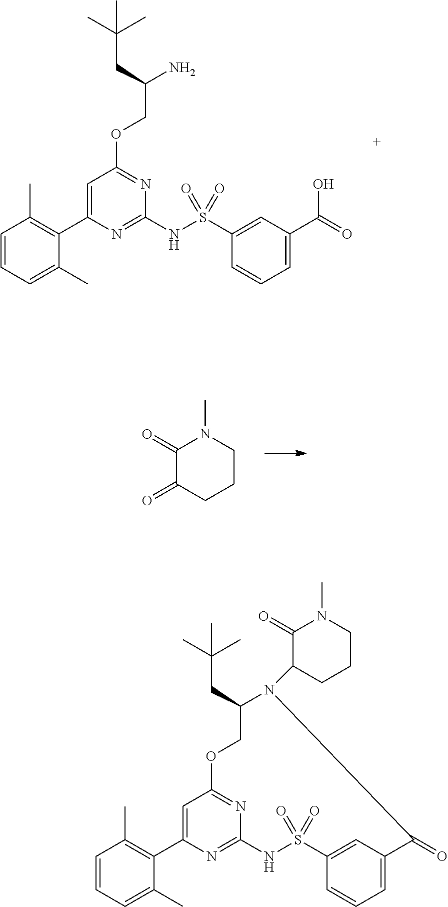

3-[[4-[(2R)-2-Amino-4,4-dimethyl-pentoxy]-6-(2,6-dimethylphenyl)pyrimidin-2-yl]sulfamoyl]benzoic acid (hydrochloride salt) (300 mg, 0.5464 mmol) was combined with 1-methylpiperidine-2,3-dione (69.47 mg, 0.5464 mmol) in DCM (5.4 mL) and stirred at room temperature for 15 minutes. Sodium triacetoxyborohydride (115.8 mg, 0.5464 mmol) was then added, followed by additional sodium triacetoxyborohydride (347.4 mg, 1.639 mmol) 15 minutes later. The reaction was allowed to stir at room temperature for an additional 60 minutes, then was quenched with a small amount of HCl (120 μL of 1 M, 0.1200 mmol), then was partially concentrated. After diluting with 1:1 DMSO/methanol and filtering, the reaction mixture was then purified by preparative HPLC (10-99% ACN in water, HCl modifier, 15 minute run) to afford a white solid. The crude solid was dissolved in DMF (5.4 mL) and mixed with 2-chloro-4,6-dimethoxy-1,3,5-triazine (95.93 mg, 0.5464 mmol). The reaction mixture was cooled in an ice bath for 10 minutes and then 4-methylmorpholine (210.2 μL, 1.912 mmol) was added and the mixture was stirred overnight. The crude mixture was diluted with 1:1 DMSO/methanol and filtering, the reaction mixture was then purified by prep HPLC (10-99% ACN in water, HCl modifier, 15 minutes run) to afford a white solid. The material was further purified by silica gel chromatography by using 0-10% MeOH in DCM to obtain a white solid (11R)-6-(2,6-dimethylphenyl)-11-(2,2-dimethylpropyl)-12-(1-methyl-2-oxo-3-piperidyl)-2,2-dioxo-9-oxa-2λ⁶-thia-3,5,12,19-tetrazatricyclo[12.3.1.14,8]nonadeca-1(18),4(19),5,7,14,16-hexaen-13-one (5.4 mg, 5%)¹H NMR (400 MHz, Methanol-d₄) δ 8.58 (d, J=1.8 Hz, 1H), 8.03 (dt, J=6.6, 2.0 Hz, 1H), 7.78-7.47 (m, 2H), 7.25 (t, J=7.6 Hz, 1H), 7.12 (d, J=7.6 Hz, 2H), 6.21 (s, 1H), 5.31 (dd, J=10.5, 4.3 Hz, 1H), 4.40-4.08 (m, 1H), 4.14-3.76 (m, 2H), 3.65-3.36 (m, 1H), 3.01 (s, 3H), 2.39 (dt, J=19.3, 11.0 Hz, 1H), 2.30 (dd, J=14.6, 5.2 Hz, 1H), 2.26-1.55 (m, 10H), 1.49 (dd, J=14.6, 2.0 Hz, 1H), 0.58 (s, 9H). ESI-MS m/z calc. 605.2672, found 606.4 (M+1)⁺; Retention time: 1.3 minutes (LC method A).

### Example 159: Preparation of Compound 508

### Step 1: (11R)-6-(2,6-Dimethylphenyl)-11-(2,2-dimethylpropyl)-12-(1-methyl-2-oxo-pyrrolidin-3-yl)-2,2-dioxo-9-oxa-2λ⁶-thia-3,5,12,19-tetrazatricyclo[12.3.1.14,8]nonadeca-1(18),4(19),5,7,14,16-hexaen-13-one

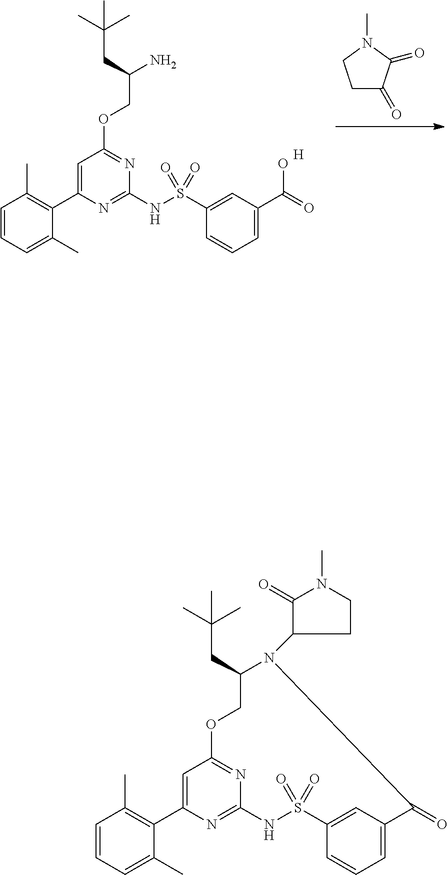

3-[[4-[(2R)-2-Amino-4,4-dimethyl-pentoxy]-6-(2,6-dimethylphenyl)pyrimidin-2-yl]sulfamoyl]benzoic acid (Hydrochloride salt) (450 mg, 0.8195 mmol) was combined with 1-methylpyrrolidine-2,3-dione (139 mg, 1.229 mmol) in DCM (2.4 mL) and stirred at room temperature for 15 minutes. Sodium triacetoxyborohydride (173.7 mg, 0.8196 mmol) was then added, followed by an additional sodium triacetoxyborohydride (521.2 mg, 2.459 mmol) 15 minutes later. The reaction was allowed to stir at room temperature for an additional 60 minutes, then was quenched with a small amount of HCl (120 μL of 1 M, 0.1200 mmol), then was partially concentrated. After diluting with 1:1 DMSO/methanol and filtering, the reaction mixture was then purified by prep HPLC (10-99ACN in water, HCl modifier, 15 minutes run) to afford a white solid, 3-[[4-[(2R)-4,4-dimethyl-2-[(1-methyl-2-oxo-pyrrolidin-3-yl)amino]pentoxy]-6-(2,6-dimethylphenyl)pyrimidin-2-yl]sulfamoyl]benzoic acid (356 mg, 71%) ESI-MS m/z calc. 609.2621, found 610.6 (M+1)⁺; Retention time: 0.45 minutes (LC method D).

3-[[4-[(2R)-4,4-Dimethyl-2-[(1-methyl-2-oxo-pyrrolidin-3-yl)amino]pentoxy]-6-(2,6-dimethylphenyl)pyrimidin-2-yl]sulfamoyl]benzoic acid (356 mg, 71%) and 2-chloro-4,6-dimethoxy-1,3,5-triazine (102.1 mg, 0.5815 mmol) were dissolved in DMF (22.5 mL) and cooled to 0° C. and stirred at this temperature for 15 minutes. To the reaction mixture, 4-methylmorpholine (225.2 μL, 2.048 mmol) was added the cooling bath was removed and stirred at room temperature overnight. The crude material was diluted with 1 mL MeOH. filtered and injected in preparative reverse phase HPLC (1-99% ACN with 5 mM HCl modifier) to obtain a white solid. This white solid was further purified by silica gel chromatography (100% DCM-5% MeOH in DCM, 30 min run) to obtain a white solid (11R)-6-(2,6-dimethylphenyl)-11-(2,2-dimethylpropyl)-12-(1-methyl-2-oxo-pyrrolidin-3-yl)-2,2-dioxo-9-oxa-2λ⁶-thia-3,5,12,19-tetrazatricyclo[12.3.1.14,8]nonadeca-1(18),4(19),5,7,14,16-hexaen-13-one (12.5 mg, 8%). ¹H NMR (400 MHz, Methanol-d₄) δ 8.62 (d, J=1.9 Hz, 1H), 8.04 (dt, J=6.7, 1.9 Hz, 1H), 7.77-7.49 (m, 2H), 7.25 (t, J=7.6 Hz, 1H), 7.12 (d, J=7.7 Hz, 2H), 6.24 (s, 1H), 5.32 (dd, J=10.6, 4.4 Hz, 1H), 4.58 (s, 1H), 4.35 (t, J=9.0 Hz, 1H), 4.20 (t, J=11.2 Hz, 1H), 4.04-3.88 (m, 1H), 3.61-3.39 (m, 2H), 2.90 (s, 3H), 2.60-2.28 (m, 2H), 2.30-1.81 (m, 7H), 1.55 (dd, J=14.7, 1.7 Hz, 1H), 0.59 (s, 9H). ESI-MS m/z calc. 591.2515, found 592.3 (M+1)⁺; Retention time: 1.28 minutes (LC method A).

### IX. Biological Activity

### A. HBE2 Assay

1. Ussing Chamber Assay of CFTR-mediated Short-circuit Currents

Ussing chamber experiments were performed using human bronchial epithelial (HBE) cells derived from CF subjects heterozygous for F508del and a minimal function CFTR mutation (F508del/MF-HBE) and cultured as previously described (Neuberger T, Burton B, Clark H, Van Goor F Methods Mol Biol 2011:741:39-54). After four days the apical media was removed, and the cells were grown at an air liquid interface for >14 days prior to use. This resulted in a monolayer of fully differentiated columnar cells that were ciliated, features that are characteristic of human bronchial airway epithelia.

To isolate the CFTR-mediated short-circuit (I_{SC}) current, F508del/MF-HBE grown on Costar® Snapwell™ cell culture inserts were mounted in an Ussing chamber and the transepithelial I_{SC} was measured under voltage-clamp recording conditions (V_{hold}=0 mV) at 37° C. The basolateral solution contained (in mM) 145 NaCl, 0.83 K₂HPO₄, 3.3 KH₂PO₄, 1.2l MgCl₂, 1.2 CaCl₂), 10 Glucose, 10 HEPES (pH adjusted to 7.4 with NaOH) and the apical solution contained (in mM) 145 NaGluconate, 1.2 MgCl₂, 1.2 CaCl₂), 10 glucose, 10 HEPES (pH adjusted to 7.4 with NaOH) and 30 μM amiloride to block the epithelial sodium channel. Forskolin (20 μM) was added to the apical surface to activate CFTR, followed by apical addition of a CFTR inhibitor cocktail consisting of BPO, GlyH-101, and CFTR inhibitor 172 (each at 20 μM final assay concentration) to specifically isolate CFTR currents. The CFTR-mediated I_{SC} (μA/cm²) for each condition was determined from the peak forskolin response to the steady-state current following inhibition.

2. Identification of Corrector Compounds

The activity of the CFTR corrector compounds on the CFTR-mediated I_{SC} was determined in Ussing chamber studies as described above. The F508del/MF-HBE cell cultures were either incubated with the corrector compounds at a range of concentrations in combination with 44 nM (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol or were incubated with the corrector compounds at a single fixed concentration of 1 and 3 μM in combination with 44 nM (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol for 18-24 hours at 37° C. and in the presence of 20% human serum. The concentration of corrector compounds with 44 nM (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol during the 18-24 hours incubations was kept constant throughout the Ussing chamber measurement of the CFTR-mediated I_{SC} to ensure compounds were present throughout the entire experiment. The efficacy and potency of the putative F508del correctors was compared to that of the known Vertex corrector, (14S)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, in combination with 18 μM Tezacaftor and 1 μM Ivacaftor.

### B. Biological Activity Data Table

Table 12 represents CFTR modulating activity for Compounds 507 and 508 generated using the assay described in this example (EC50: +++ is <1 μM; ++ is 1-<3 μM; + is 3-<30 μM; and ND is “not detected in this assay.” % Activity: +++ is >60%; ++ is 30-60%; + is <300%).

TABLE 12
Biological Activity Data

| | | HBE2 Activity at |
|---|---|---|
| Compound No. | Structure | 3 μM |
| 507 | 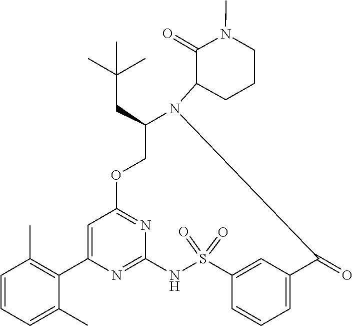 [SMILES: Cc1cccc(C)c1-c1cc2nc(n1)NS(=O)(=O)c1cccc(c1)C(=O)N(C1CCCN(C)C1=O)[C@H](CC(C)(C)C)CO2] | +++ |
| 508 | 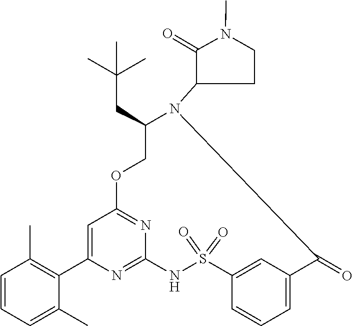 [SMILES: Cc1cccc(C)c1-c1cc2nc(n1)NS(=O)(=O)c1cccc(c1)C(=O)N(C1CCN(C)C1=O)[C@H](CC(C)(C)C)CO2] | +++ |

### X. Synthesis of (6R,12R)-17-amino-12-methyl-6,15-bis(trifluoromethyl)-13,19-dioxa-3,4,18-triazatricyclo [12.3.1.12,5] nonadeca-1(18),2,4,14,16-pentaen-6-ol

### A. General Methods

Reagents and starting materials were obtained by commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz and 28860 points of acquisition. FID were zero-filled to 32 k points and a line broadening of 0.3 Hz was applied before Fourier transform. ¹⁹F NMR spectra were recorded at 282 MHz using a 30 degree pulse angle, a spectral width of 100 kHz and 59202 points were acquired. FID were zero-filled to 64 k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz and 128 k points of acquisition. FID were zero-filled to 256 k points and a line broadening of 0.3 Hz was applied before Fourier transform. ¹⁹F NMR spectra were recorded at 377 MHz using a 30 deg pulse angle, a spectral width of 89286 Hz and 128 k points were acquired. FID were zero-filled to 256 k points and a line broadening of 0.3 Hz was applied before Fourier transform.

NMR spectra were also recorded on a Bruker AC 250 MHz instrument equipped with a: 5 mm QNP(H1/C13/F19/P31) probe (type: 250-SB, s #23055/0020) or on a Varian 500 MHz instrument equipped with a ID PFG, 5 mm, 50-202/500 MHz probe (model/part #99337300).

Unless stated to the contrary in the following examples, final purity of compounds was determined by reversed phase UPLC using an Acquity UPLC BEH C₁₈ column (50×2.1 mm, 1.7 m particle) made by Waters (pn: 186002350), and a dual gradient run from 1-99% mobile phase B over 3.0 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+1]+species obtained using a single quadrupole mass spectrometer equipped with an electrospray ionization (ESI) source capable of achieving a mass accuracy of 0.1 Da and a minimum resolution of 1000 (no units on resolution) across the detection range.

Solid-state NMR (SSNMR) spectra were recorded on a Bruker-Biospin 400 MHz wide-bore spectrometer equipped with Bruker-Biospin 4 mm HFX probe. Samples were packed into 4 mm ZrO2 rotors and spun under Magic Angle Spinning (MAS) condition with spinning speed typically set to 12.5 kHz. The proton relaxation time was measured using ¹H MAS Ti saturation recovery relaxation experiment in order to set up proper recycle delay of the ¹³C cross-polarization (CP) MAS experiment. The fluorine relaxation time was measured using ¹⁹F MAS Ti saturation recovery relaxation experiment in order to set up proper recycle delay of the ¹⁹F MAS experiment. The CP contact time of carbon CPMAS experiment was set to 2 ms. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 kHz.

### B. Procedures for the Synthesis of Intermediates

### Intermediate 1: Preparation of methyl 3-[bis(tert-butoxycarbonyl)amino]-6-bromo-5-(trifluoromethyl)pyridine-2-carboxylate

### Step 1: Methyl 3-(benzhydrylideneamino)-5-(trifluoromethyl)pyridine-2-carboxylate

A mixture of methyl 3-chloro-5-(trifluoromethyl)pyridine-2-carboxylate (47.3 g, 197.43 mmol), diphenylmethanimine (47 g, 259.33 mmol), Xantphos (9.07 g, 15.675 mmol), and cesium carbonate (131 g, 402.06 mmol) in dioxane (800 mL) was degassed with bubbling nitrogen for 30 minutes. Pd(OAc)₂ (3.52 g, 15.679 mmol) was added and the system was purged with nitrogen three times. The reaction mixture was heated at 100° C. for 18 h. The reaction was cooled to room temperature and filtered on a pad of Celite. The cake was washed with EtOAc and solvents were evaporated under reduced pressure to give methyl 3-(benzhydrylideneamino)-5-(trifluoromethyl)pyridine-2-carboxylate (90 g, 84%) as yellow solid. ESI-MS m/z calc. 384.10855, found 385.1 (M+1)⁺; Retention time: 2.24 minutes. LCMS Method: Kinetex C₁₈ 4.6×50 mm 2.6 μM, 2.0 mL/min, 95% H₂O (0.1% formic acid)⁺5% acetonitrile (0.1% formic acid) to 95% acetonitrile (0.1% formic acid) gradient (2.0 min) then held at 95% acetonitrile (0.1% formic acid) for 1.0 min.

### Step 2: Methyl 3-amino-5-(trifluoromethyl)pyridine-2-carboxylate

To a suspension of methyl 3-(benzhydrylideneamino)-5-(trifluoromethyl)pyridine-2-carboxylate (65 g, 124.30 mmol) in methanol (200 mL) was added HCl (3 M in methanol) (146 mL of 3 M, 438.00 mmol). The mixture was stirred at room temperature for 1.5 hour then the solvent was removed under reduced pressure. The residue was taken up in ethyl acetate (2 L) and dichloromethane (500 mL). The organic phase was washed with 5% aqueous sodium bicarbonate solution (3×500 mL) and brine (2×500 mL), dried over anhydrous sodium sulfate, filtered and the solvent was removed under reduced pressure. The residue was triturated with heptanes (2×50 mL) and the mother liquors were discarded. The solid obtained was triturated with a mixture of dichloromethane and heptanes (1:1, 40 mL) and filtered to afford methyl 3-amino-5-(trifluoromethyl)pyridine-2-carboxylate (25.25 g, 91%) as yellow solid. ¹H NMR (300 MHz, CDCl₃) δ 8.24 (s, 1H), 7.28 (s, 1H), 5.98 (br. s, 2H), 4.00 (s, 3H) ppm. 19F NMR (282 MHz, CDCl₃) δ-63.23 (s, 3F) ppm. ESI-MS m/z calc. 220.046, found 221.1 (M+1)⁺; Retention time: 1.62 minutes. LCMS Method: Kinetex Polar C₁₈ 3.0×50 mm 2.6 μm, 3 min, 5-95% acetonitrile in H₂O (0.1% formic acid) 1.2 mL/min.

### Step 3: Methyl 3-amino-6-bromo-5-(trifluoromethyl)pyridine-2-carboxylate

To a solution of methyl 3-amino-5-(trifluoromethyl)pyridine-2-carboxylate (18.75 g, 80.91 mmol) in acetonitrile (300 mL) at 0° C. was added portion wise N-bromosuccinimide (18.7 g, 105.3 mmol). The mixture was stirred overnight at 25° C. Ethyl acetate (1000 mL) was added. The organic layer was washed with 10% sodium thiosulfate solution (3×200 mL) which were back extracted with ethyl acetate (2×200 mL). The combined organic extracts were washed with saturated sodium bicarbonate solution (3×200 mL), brine (200 mL), dried over sodium sulfate and concentrated in vacuo to provide methyl 3-amino-6-bromo-5-(trifluoromethyl)pyridine-2-carboxylate (25.46 g, 98%). ¹H NMR (300 MHz, CDCl₃) δ 3.93-4.03 (m, 3H), 6.01 (br. s., 2H), 7.37 (s, 1H) ppm. ¹⁹F NMR (282 MHz, CDCl₃) ppm −64.2 (s, 3F). ESI-MS m/z calc. 297.9565, found 299.0 (M+1)⁺; Retention time: 2.55 minutes. LCMS Method: Kinetex C₁₈ 4.6×50 mm 2.6 μM. Temp: 45° C., Flow: 2.0 mL/min, Run Time: 6 min. Mobile Phase: Initial 95% H₂O (0.1% formic acid) and 5% acetonitrile (0.1% formic acid) linear gradient to 95% acetonitrile (0.1% formic acid) for 4.0 min then held at 95% acetonitrile (0.1% formic acid) for 2.0 min.

### Step 4: Methyl 3-[bis(tert-butoxycarbonyl)amino]-6-bromo-5-(trifluoro methyl)pyridine-2-carboxylate

A mixture of methyl 3-amino-6-bromo-5-(trifluoromethyl)pyridine-2-carboxylate (5 g, 15.549 mmol), (Boc)₂O (11 g, 11.579 mL, 50.402 mmol), DMAP (310 mg, 2.5375 mmol) and CH₂Cl₂ (150 mL) was stirred at room temperature overnight. The reaction mixture was concentrated under reduced pressure and purification by silica gel chromatography (0-15% ethyl acetate in heptane) provided methyl 3-[bis(tert-butoxycarbonyl)amino]-6-bromo-5-(trifluoromethyl)pyridine-2-carboxylate (6.73 g, 87%) as light yellow solid. ¹H NMR (300 MHz, CDCl₃) δ 1.42 (s, 18H), 3.96 (s, 3H), 7.85 (s, 1H) ppm. ¹⁹F NMR (282 MHz, CDCl₃) δ-63.9 (s, 3F) ppm. ESI-MS m/z calc. 498.06134, Retention time: 2.34 minutes. LCMS Method: Kinetex C₁₈ 4.6×50 mm 2.6 μM. Temp: 45° C., Flow: 2.0 mL/min, Run Time: 3 min. Mobile Phase: Initial 95% H₂O (0.1% formic acid) and 5% acetonitrile (0.1% formic acid) linear gradient to 95% acetonitrile (0.1% formic acid) for 2.0 min then held at 95% acetonitrile (0.1% formic acid) for 1.0 min.

### Intermediate 2: Preparation of 6-bromo-3-(tert-butoxycarbonylamino)-5-(trifluoromethyl)pyridine-2-carboxylic acid

### Step 1: 6-Bromo-3-(tert-butoxycarbonylamino)-5-(trifluoromethyl)pyridine-2-carboxylic acid

To a mixture of methyl 3-[bis(tert-butoxycarbonyl)amino]-6-bromo-5-(trifluoromethyl)pyridine-2-carboxylate (247 g, 494.7 mmol) in THE (1.0 L) was added a solution of LiOH (47.2 g, 1.971 mol) in water (500 mL). The mixture was stirred at ambient temperature for 18 h affording a yellow slurry. The mixture was cooled with an ice-bath and slowly acidified with HCl (1000 mL of 2 M, 2.000 mol) keeping the reaction temperature <15° C. The mixture was diluted with heptane (1.5 L), mixed and the organic phase separated. The aqueous phase was extracted with heptane (500 mL). The combined organic phases were washed with brine, dried over MgSO₄, filtered and concentrated in vacuo. The crude oil was dissolved in heptane (600 mL), seeded and stirred at ambient temperature for 18 h affording a thick slurry. The slurry was diluted with cold heptane (500 mL) and the precipitate collected using a medium frit. The filter cake was washed with cold heptane and air dried for 1 h, then in vacuo at 45° C. for 48 h to afford 6-bromo-3-(tert-butoxycarbonylamino)-5-(trifluoromethyl)pyridine-2-carboxylic acid (158.3 g, 83%). ¹H NMR (400 MHz, DMSO-d₆) δ 10.38 (s, 1H), 9.01 (s, 1H), 1.50 (s, 9H) ppm. ESI-MS m/z calc. 383.99326, found 384.9 (M+1)⁺; Retention time: 2.55 minutes. LCMS Method Detail: Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=acetonitrile (0.035% CF₃CO₂H). Flow rate=1.2 mL/min, injection volume=1.5 μL, and column temperature=60° C.

### Intermediate 3: Preparation of 2-Benzyloxy-2-(trifluoromethyl)hex-5-enoic acid

### Step 1: Ethyl 2-hydroxy-2-(trifluoromethyl)hex-5-enoate

To a solution of ethyl 3,3,3-trifluoro-2-oxo-propanoate (25.15 g, 147.87 mmol) in Et₂O (270 mL) at −78° C. was added bromo(but-3-enyl)magnesium in THE (190 mL of 0.817 M, 155.23 mmol) dropwise over a period of 1.5 h (inner temperature−72° C. to −76° C.). The mixture was stirred at −78° C. for 20 min. The dry ice-acetone bath was removed. The mixture was slowly warm to 5° C. during 1 h, added to a mixture of 1 N aqueous HCl (170 mL) and crushed ice (150 g) (pH=4). The two layers were separated. The organic layer was concentrated, and the residue was combined with aqueous phase and extracted with EtOAc (2×150 mL). The combined organic phase was washed with 5% aqueous NaHCO₃ (50 mL) and brine (20 mL), dried with Na₂SO₄. The mixture was filtered and concentrated, and co-evaporated with THE (2×40 mL) to give ethyl 2-hydroxy-2-(trifluoromethyl)hex-5-enoate (37.44 g, 96%) as colorless oil. ¹H NMR (300 MHz, CDCl₃) δ 5.77 (ddt, J=17.0, 10.4, 6.4 Hz, 1H), 5.15-4.93 (m, 2H), 4.49-4.28 (m, 2H), 3.88 (s, 1H), 2.35-2.19 (m, 1H), 2.17-1.89 (m, 3H), 1.34 (t, J=7.0 Hz, 3H) ppm. ¹⁹F NMR (282 MHz, CDCl₃) δ-78.74 (s, 3F) ppm.

### Step 2: Ethyl 2-benzyloxy-2-(trifluoromethyl)hex-5-enoate

To a solution of ethyl 2-hydroxy-2-(trifluoromethyl)hex-5-enoate (24.29 g, 87.6% purity, 94.070 mmol) in DMF (120 mL) at 0° C. was added NaH (60% in mineral oil, 5.64 g, 141.01 mmol) portion-wise. The mixture was stirred at 0° C. for 10 min. Benzyl bromide (24.13 g, 141.08 mmol) and TBAI (8.68 g, 23.500 mmol) were added. The mixture was stirred at room temperature overnight. NH₄C₁ (3 g, 0.6 eq) was added. The mixture was stirred for 10 min. 30 mL of EtOAc was added, then ice-water was added (400 g). The mixture was extracted with CH₂Cl₂ and the combined organic layers were concentrated. Purification by silica gel chromatography (0-20% CH₂Cl₂ in heptanes) provided ethyl 2-benzyloxy-2-(trifluoromethyl)hex-5-enoate (26.05 g, 88%) as pink oil. ¹H NMR (300 MHz, CDCl₃) δ 1.34 (t, J=7.2 Hz, 3H), 2.00-2.19 (m, 3H), 2.22-2.38 (m, 1H), 4.33 (q, J=7.2 Hz, 2H), 4.64 (d, J=10.6 Hz, 1H), 4.84 (d, J=10.9 Hz, 1H), 4.91-5.11 (m, 2H), 5.62-5.90 (m, 1H), 7.36 (s, 5H) ppm. 19F NMR (282 MHz, CDCl₃) δ-70.5 (s, 3F) ppm. ESI-MS m/z calc. 316.12863, found 317.1 (M+1)⁺; Retention time: 2.47 minutes. LCMS Method: Kinetex C₁₈ 4.6×50 mm 2.6 μM. Temp: 45° C., Flow: 2.0 mL/min, Run Time: 3 min. Mobile Phase: Initial 95% H₂O (0.1% formic acid) and 5% acetonitrile (0.1% formic acid) linear gradient to 95% acetonitrile (0.1% formic acid) for 2.0 min then held at 95% acetonitrile (0.1% formic acid) for 1.0 min.

### Step 3: 2-Benzyloxy-2-(trifluoromethyl)hex-5-enoic acid

A solution of sodium hydroxide (7.86 g, 196.51 mmol) in water (60 mL) was added to a solution of ethyl 2-benzyloxy-2-(trifluoromethyl)hex-5-enoate (24.86 g, 78.593 mmol) in methanol (210 mL). The reaction was heated at 50° C. overnight. The reaction was concentrated to remove methanol, diluted with water (150 mL) and the carboxylate sodium salt was washed with heptane (1×100 mL). The aqueous solution was acidified to pH=2 with aqueous 3N solution of HCl. The carboxylic acid was extracted with dichloromethane (3×100 mL) and dried over sodium sulfate. The solution was filtered and concentrated to give 2-benzyloxy-2-(trifluoromethyl)hex-5-enoic acid (22.57 g, 97%) as pale yellow oil. ¹H NMR (300 MHz, DMSO-d₆) δ 14.31 (br. s., 1H), 7.55-7.20 (m, 5H), 5.93-5.70 (m, 1H), 5.17-4.91 (m, 2H), 4.85-4.68 (m, 1H), 4.67-4.55 (m, 1H), 2.32-1.94 (m, 4H) ppm. ¹⁹F NMR (282 MHz, DMSO-d₆) δ-70.29 (s, 3F) ppm. ESI-MS m/z calc. 288.09732, found 287.1 (M-1); Retention time: 3.1 minutes. LCMS Method: Kinetex Polar C₁₈ 3.0×50 mm 2.6 μm, 6 min, 5-95% acetonitrile in H₂O (0.1% formic acid) 1.2 mL/min.

### Intermediate 4: Preparation of (2R)-2-benzyloxy-2-(trifluoromethyl)hex-5-enoic acid

### Step-1: (2R)-2-Benzyloxy-2-(trifluoromethyl)hex-5-enoic acid; (R)-4-quinolyl-[(2S,4S)-5-vinylquinuclidin-2-yl]methanol

To a N₂ purged jacketed reactor set to 20° C. was added isopropyl acetate (IPAC, 100 L, 0.173 M, 20 Vols), followed by previously melted 2-benzyloxy-2-(trifluoromethyl)hex-5-enoic acid (5.00 kg, 17.345 mol) and cinchonidine (2.553 kg, 8.67 mol) made into a slurry with minor amount of the reaction solvent. The reactor was set to ramp internal temperature to 80° C. over 1 hour, with solids going in solution upon heating to set temperature, then the solution was held at temperature for at least 10 minutes, then cooled to 70° C. held and seeded with chiral salt (50 g, 1.0% by wt). The mixture was stirred for 10 minutes, then ramped to 20° C. internal temperature over 4 hours, then held overnight at 20° C. The mixture was filtered, cake washed with isopropyl acetate (10.0 L, 2.0 vols) and dried under vacuum. The cake was then dried in vacuo (50° C., vacuum) to afford 4.7 kg of salt. The resulting solid salt was returned to the reactor by making a slurry with a portion of isopropyl acetate (94 L, 20 vol based on current salt wt), and pumped into reactor and stirred. The mixture was then heated to 80° C. internal, stirred hot slurry for at least 10 minutes, then ramped to 20° C. over 4-6 h, then stirred overnight at 20° C. The material was then filtered and cake washed with isopropyl acetate (9.4 L, 2.0 vol), pulled dry, cake scooped out and dried in vacuo (50° C., vacuum) to afford 3.1 kg of solid. The solid (3.1 kg) and isopropyl acetate (62 L, 20 vol based on salt solid wt) was slurried and added to a reactor, stirred under N₂ purge and heated to 80° C. and held at temperature at least 10 minutes, then ramped to 20° C. over 4-6 hours, then stirred overnight. The mixture was filtered, cake washed with isopropyl acetate (6.2 L, 2 vol), pulled dry, scooped out and dried in vacuo (50° C., vac) to afford 2.25 kg of solid salt. The solid (2.25 kg) and isopropyl acetate (45 L, 20 vol based on salt solid wt) was slurried and added to a reactor, stirred under N₂ purge and heated to 80° C., held at temperature at least 10 minutes, then ramped to 20° C. over 4-6 hours, then stirred overnight. The mixture was filtered, cake washed with isopropyl acetate (4.5 L, 2 vol), pulled dry, scooped out and dried in vacuo (50° C. to afford (2R)-2-benzyloxy-2-(trifluoromethyl)hex-5-enoic acid; (R)-4-quinolyl-[(2S,4S)-5-vinylquinuclidin-2-yl]methanol (1.886 kg, >98.0% ee) as off-white to tan solid. Chiral purity was determined by Agilent 1200 HPLC instrument using Phenomenex Lux i-Amylose-3 column (3 μm, 150×4.6 mm) and a dual, isocratic gradient run 30% to 70% mobile phase B over 20.0 minutes. Mobile phase A=H₂O (0.1% CF₃CO₂H). Mobile phase B=MeOH (0.1% CF₃CO₂H). Flow rate=1.0 mL/min, injection volume=2 μL, and column temperature=30° C., sample concentration: 1 mg/mL in 60% acetonitrile/40% water.

### Step 2: (2R)-2-Benzyloxy-2-(trifluoromethyl)hex-5-enoic acid

A suspension of (2R)-2-benzyloxy-2-(trifluoromethyl)hex-5-enoic acid; (R)-4-quinolyl-[(2S,4S)-5-vinylquinuclidin-2-yl]methanol (50 g, 87.931 mmol) in ethyl acetate (500.00 mL) was treated with an aqueous solution of hydrochloric acid (200 mL of 1 M, 200.00 mmol). After stirring 15 minutes at room temperature, the two phases were separated. The aqueous phase was extracted twice with ethyl acetate (200 mL). The combined organic layer was washed with 1 N HCl (100 mL). The organic layer was dried over sodium sulfate, filtered and concentrated. The material was dried over high vacuum overnight to give (2R)-2-benzyloxy-2-(trifluoromethyl)hex-5-enoic acid (26.18 g, 96%) as pale brown oil. ¹H NMR (400 MHz, CDCl₃) δ 7.46-7.31 (m, 5H), 5.88-5.73 (m, 1H), 5.15-4.99 (m, 2H), 4.88 (d, J=10.3 Hz, 1H), 4.70 (d, J=10.3 Hz, 1H), 2.37-2.12 (m, 4H) ppm. ¹⁹F NMR (377 MHz, CDCl₃) δ-71.63 (br s, 3F) ppm. ESI-MS m/z calc. 288.0973, found 287.0 (M-1)−; Retention time: 2.15 minutes. LCMS Method: Kinetex Polar C₁₈ 3.0×50 mm 2.6 μm, 3 min, 5-95% acetonitrile in H₂O (0.1% formic acid) 1.2 mL/min.

### Intermediate 5: Preparation of (2R)-2-benzyloxy-2-(trifluoromethyl)hex-5-enehydrazide

### Step 1: tert-Butyl N-[[(2R)-2-benzyloxy-2-(trifluoromethyl)hex-5-enoyl]amino]carbamate

To a solution of (2R)-2-benzyloxy-2-(trifluoromethyl)hex-5-enoic acid (365 g, 1.266 mol) in DMF (2 L) was added HATU (612 g, 1.610 mol) and DIEA (450 mL, 2.584 mol) and the mixture was stirred at ambient temperature for 10 min. To the mixture was added tert-butyl N-aminocarbamate (200 g, 1.513 mol) (slight exotherm upon addition) and the mixture was stirred at ambient temperature for 16 h. The reaction was poured into ice water (5 L). The resultant precipitate was collected by filtration and washed with water. The solid was dissolved in EtOAc (2 L) and washed with brine. The organic phase was dried over MgSO₄, filtered and concentrated in vacuo. The oil was diluted with EtOAc (500 mL) followed by heptane (3 L) and stirred at ambient temperature for several hours affording a thick slurry. The slurry was diluted with additional heptane and filtered to collect fluffy white solid (343 g). The filtrate was concentrated and purification by silica gel chromatography (0-40% EtOAc/hexanes) provided tert-butyl N-[[(2R)-2-benzyloxy-2-(trifluoromethyl)hex-5-enoyl]amino]carbamate (464 g, 91%, combined with product from crystallization). ESI-MS m/z calc. 402.17664, found 303.0 (M+1-Boc)⁺; Retention time: 2.68 minutes. Final purity was determined by reversed phase UPLC using an Acquity UPLC BEH C₁₈ column (50×2.1 mm, 1.7 m particle) made by Waters (pn: 186002350) and a dual gradient run from 1-99% mobile phase B over 4.5 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

### Step 2: (2R)-2-Benzyloxy-2-(trifluoromethyl)hex-5-enehydrazide

To a solution of tert-butyl N-[[(2R)-2-benzyloxy-2-(trifluoromethyl)hex-5-enoyl]amino]carbamate (464 g, 1.153 mol) in DCM (1.25 L) and was added HCl (925 mL of 4 M, 3.700 mol) and the mixture stirred at ambient temperature for 20 h. The mixture was concentrated in vacuo removing most of the DCM. The mixture was diluted with isopropyl acetate (1 L) and basified to pH=6 with NaOH (140 g of 50% w/w, 1.750 mol) in 1 L of ice water. The organic phase was separated and washed with 1 L of brine and the combined aqueous phases were extracted with isopropyl acetate (1 L). The combined organic phases were dried over MgSO₄, filtered and concentrated in vacuo affording a dark yellow oil of (2R)-2-benzyloxy-2-(trifluoromethyl)hex-5-enehydrazide (358 g, quant.). ¹H NMR (400 MHz, CDCl₃) δ 8.02 (s, 1H), 7.44-7.29 (m, 5H), 5.81 (ddt, J=16.8, 10.1, 6.4 Hz, 1H), 5.13-4.93 (m, 2H), 4.75 (dd, J=10.5, 1.5 Hz, 1H), 4.61 (d, J=10.5 Hz, 1H), 3.78 (s, 2H), 2.43 (ddd, J=14.3, 11.0, 5.9 Hz, 1H), 2.26-1.95 (m, 3H) ppm. ESI-MS m/z calc. 302.1242, found 303.0 (M+1)⁺; Retention time: 2.0 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

### Intermediate 6: Preparation of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

### Step 1: tert-Butyl N-[2-[[[(2R)-2-benzyloxy-2-(trifluoromethyl)hex-5-enoyl]amino]carbamoyl]-6-bromo-5-(trifluoromethyl)-3-pyridyl]carbamate

To a mixture of 6-bromo-3-(tert-butoxycarbonylamino)-5-(trifluoromethyl)pyridine-2-carboxylic acid (304 g, 789.3 mmol) and (2R)-2-benzyloxy-2-(trifluoromethyl)hex-5-enehydrazide (270 g, 893.2 mmol) in EtOAc (2.25 L) at ambient temperature was added DIEA (425 mL, 2.440 mol). To the mixture was slowly added T3P (622 g of 50% w/w, 977.4 mmol) using an ice-water bath to keep the temperature <35° C. (temperature rose to 34° C.) and the reaction mixture was stirred at ambient temperature for 18 h. Added additional DIEA (100 mL, 574.1 mmol) and T3P (95 g, 298.6 mmol) and stirred at ambient temperature for 2 days. Starting material was still observed and an additional T3P (252 g, 792 mmol) was added and stirred for 5 days. The reaction was quenched with the slow addition of water (2.5 L) and the mixture stirred for 30 min. The organic phase was separated, and the aqueous phase extracted with EtOAc (2 L). The combined organic phases were washed with brine, dried over MgSO₄, filtered and concentrated in vacuo. The crude product was dissolved in MTBE (300 mL) and diluted with heptane (3 L), the mixture stirred at ambient temperature for 12 h affording a light yellow slurry. The slurry was filtered, and the resultant solid was air dried for 2 h, then in vacuo at 40° C. for 48 h. The filtrate was concentrated in vacuo and purified by silica gel chromatography (0-20% EtOAc/hexanes) and combined with material obtained from crystallization providing tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (433 g, 82%). ¹H NMR (400 MHz, DMSO) δ 11.07 (s, 1H), 10.91 (s, 1H), 10.32 (s, 1H), 9.15 (s, 1H), 7.53-7.45 (m, 2H), 7.45-7.28 (m, 3H), 5.87 (ddt, J=17.0, 10.2, 5.1 Hz, 1H), 5.09 (dq, J=17.1, 1.3 Hz, 1H), 5.02 (dd, J=10.3, 1.9 Hz, 1H), 4.84 (q, J=11.3 Hz, 2H), 2.37-2.13 (m, 4H), 1.49 (s, 9H) ppm. ESI-MS m/z calc. 668.1069, found 669.0 (M+1)⁺; Retention time: 3.55 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

### Step 2: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

To a solution of tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (240 g, 358.5 mmol) in anhydrous acetonitrile (1.5 L) under nitrogen was added DIEA (230 mL, 1.320 mol) and the orange solution heated to 70° C. To the mixture was added p-toluenesulfonyl chloride (80.5 g, 422.2 mmol) in 3 equal portions over 1 h. The mixture was stirred at 70° C. for 9 h then additional p-toluenesulfonyl chloride (6.5 g, 34.09 mmol) was added. The mixture was stirred for a total of 24 h then allowed to cool to ambient temperature. Acetonitrile was removed in vacuo affording a dark orange oil which was diluted with EtOAc (1.5 L) and water (1.5 L). The organic phase was separated and washed with 500 mL of 1M HCl, 500 mL of brine, dried over MgSO₄, filtered and concentrated in vacuo. Purification by silica gel chromatography (0-20% EtOAc/hexanes) provided tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (200 g, 86%). ¹H NMR (400 MHz, DMSO) δ 10.11 (s, 1H), 9.10 (s, 1H), 7.55-7.48 (m, 2H), 7.47-7.28 (m, 3H), 5.87 (ddt, J=16.7, 10.2, 6.4 Hz, 1H), 5.11 (dt, J=17.2, 1.7 Hz, 1H), 5.01 (dt, J=10.2, 1.5 Hz, 1H), 4.74 (d, J=10.6 Hz, 1H), 4.65 (d, J=10.6 Hz, 1H), 2.55-2.42 (m, 2H), 2.30 (qd, J=11.3, 10.3, 6.9 Hz, 2H), 1.52 (s, 9H) ppm. ESI-MS m/z calc. 650.0963, found 650.0 (M+1)⁺; Retention time: 3.78 minutes. Final purity was determined by reversed phase UPLC using an Acquity UPLC BEH C₁₈ column (50×2.1 mm, 1.7 μm particle) made by Waters (pn: 186002350), and a dual gradient run from 1-99% mobile phase B over 4.5 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

### Intermediate 7: Preparation of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate

### Step 1: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate

To a solution of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (222 g, 340.8 mmol) in MTBE (1.333 L) was added DIPEA (65.3 mL, 374.9 mmol) followed DMAP (2.09 g, 17.11 mmol). Added a solution of di-tert-butyl dicarbonate (111.6 g, 511.3 mmol) in MTBE (250 mL) over approx. 8 minutes, and the resulting mixture was stirred for additional 30 min. Added 1 L of water and separated the layers. The organic layer was washed with KHSO₄ (886 mL of 0.5 M, 443.0 mmol), 300 mL brine, dried with MgSO₄ and most (>95%) of the MTBE was evaporated by rotary evaporation at 45° C., leaving a thick oil. Added 1.125 L of heptane, spun in the 45° C. rotovap bath until dissolved, then evaporated out 325 mL of solvent by rotary evaporation. The rotovap bath temp was allowed to drop to room temperature and product started crystallizing out during the evaporation. Then put the flask in a −20° C. freezer overnight. The resultant solid was filtered and washed with cold heptane and dried at room temperature for 3 days to give tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (240.8 g, 94%). ¹H NMR (400 MHz, Chloroform-d) δ 7.95 (s, 1H), 7.52-7.45 (m, 2H), 7.44-7.36 (m, 2H), 7.36-7.29 (m, 1H), 5.83-5.67 (m, 1H), 5.08-5.00 (m, 1H), 5.00-4.94 (m, 1H), 4.79 (d, J=10.4 Hz, 1H), 4.64 (d, J=10.4 Hz, 1H), 2.57-2.26 (m, 3H), 2.26-2.12 (m, 1H), 1.41 (s, 18H) ppm. ESI-MS m/z calc. 750.14874, found 751.1 (M+1)⁺; Retention time: 3.76 minutes. Final purity was determined by reversed phase UPLC using an Acquity UPLC BEH Cis column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B═CH₃CN (0.035% CF₃CO₂H). Flow rate=1.2 mL/min, injection volume=1.5 μL, and column temperature=60° C.

### Intermediate 8: Preparation of tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate

### Step 1: tert-Butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate

tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (280 g, 372.6 mmol) was dissolved in DMSO (1.82 L) (yellow solution) and treated with cesium acetate (215 g, 1.120 mol) under stirring at room temperature. The yellow suspension was heated at 80° C. for 5 h. The reaction mixture was cooled to room temperature and added to a stirred cold emulsion of water (5.5 L) with 1 kg ammonium chloride dissolved in it and a 1:1 mixture of MTBE and heptane (2 L) (in 20 L). The phases were separated and the organic phase washed water (3×3 L) and with brine (1×2.5 L). The organic phase was dried with MgSO₄, filtered and concentrated under reduced pressure. The resultant yellow solution was diluted with heptane (˜1 L) and seeded with tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate and stirred on the rotovap at 100 mbar pressure at room temperature for 1.5 h. The solid mass was stirred mechanically for 2 h at room temperature, resultant thick fine suspension was filtered, washed with dry ice cold heptane and dried under vacuum at 45° C. with a nitrogen bleed for 16 h to give tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (220 g, 85%) as an off white solid. ¹H NMR (400 MHz, DMSO-d₆) δ 13.28 (s, 1H), 8.43 (s, 1H), 7.58-7.26 (m, 5H), 5.85 (ddt, J=16.8, 10.3, 6.5 Hz, 1H), 5.10 (dq, J=17.2, 1.6 Hz, 1H), 5.01 (dq, J=10.2, 1.3 Hz, 1H), 4.76 (d, J=11.0 Hz, 1H), 4.65 (d, J=11.0 Hz, 1H), 2.55 (dd, J=9.6, 5.2 Hz, 2H), 2.23 (td, J=13.2, 10.0, 5.7 Hz, 2H), 1.27 (d, J=3.8 Hz, 18H) ppm. ESI-MS m/z calc. 688.23315, found 689.0 (M+1)⁺; Retention time: 3.32 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B ═CH₃CN (0.035% CF₃CO₂H). Flow rate=1.2 mL/min, injection volume=1.5 μL, and column temperature=60° C.

### C. Preparation of (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol

### Step 1: tert-Butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate

Dissolved tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (159.3 g, 231.3 mmol) and triphenylphosphine (72.9 g, 277.9 mmol) in toluene (1 L), then added (2S)-pent-4-en-2-ol (28.7 mL, 278.9 mmol). Heated this mixture to 45° C., then added DIAD (58.3 mL, 296.1 mmol) (exotherm) slowly over 40 min. For the next approximately 2 h, the mixture was cooled to room temperature. During this cooling period, after the first 10 minutes, triphenylphosphine (6.07 g, 23.14 mmol) was added. After a further 1 h, additional triphenylphosphine (3.04 g, 11.59 mmol) was added. After a further 23 min, DIAD (2.24 mL, 11.57 mmol) was added. After the ˜2 h cooling to room temperature period, the mixture was cooled to 15° C., and seed crystals of DIAD-triphenylphosphine oxide complex were added which caused precipitation to occur, then added 1000 mL heptane. Stored the mixture at −20° C. for 3 days. Filtered out and discarded the precipitate and concentrated the filtrate to give a red residue/oil. Dissolved the residue in 613 mL heptane at 45° C., then cooled to 0° C., seeded with DIAD-triphenylphosphine oxide complex, stirred at 0° C. for 30 min, then filtered the solution. The filtrate was concentrated to a smaller volume, then loaded onto a 1.5 kg silica gel column (column volume=2400 mL, flow rate=600 mL/min). Ran a gradient of 1% to 6% EtOAc in hexanes over 32 minutes (8 column volumes), then held at 6% EtOAc in hexanes until the product finished eluting which gave tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (163.5 g, 93%). ¹H NMR (400 MHz, Chloroform-d) δ 7.82 (s, 1H), 7.43-7.27 (m, 5H), 5.88-5.69 (m, 2H), 5.35 (h, J=6.2 Hz, 1H), 5.16-4.94 (m, 4H), 4.81 (d, J=10.7 Hz, 1H), 4.63 (d, J=10.7 Hz, 1H), 2.58-2.15 (m, 6H), 1.42 (s, 18H), 1.36 (d, J=6.2 Hz, 3H) ppm. ESI-MS m/z calc. 756.2958, found 757.3 (M+1)⁺; Retention time: 4.0 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 2: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture)

The following reaction was run, split equally between two, 12 L reaction flasks run in parallel. Mechanical stirring was employed, and reactions were subjected to a constant nitrogen gas purge using a coarse porosity gas dispersion tube. To each flask was added tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (54 g, 71.36 mmol in each flask) dissolved in DCE (8 L in each flask) and both flasks were strongly purged with nitrogen at room temperature. Both flasks were heated to 62° C. and Grubbs 1^{st} Generation Catalyst (9 g, 10.94 mmol in each flask) was added to each reaction and stirred at 400 rpm while setting an internal temperature control to 75° C. with strong nitrogen purging (both reactions reached ˜75° C. after approximately 20 min). After 5 h 15 min, the internal temperature control was set to 45° C. After approximately 2 h, 2-sulfanylpyridine-3-carboxylic acid (11 g, 70.89 mmol in each flask) was added to each flask followed by triethylamine (10 mL, 71.75 mmol in each flask). On completion of addition, the nitrogen purge was turned off and both reaction flasks were stirred at 45° C. open to air overnight. The reactions were then removed from heat and 130 g of silica gel was added to each reaction and each was stirred at room temperature. After approximately 2 h, the green mixtures were combined and filtered over Celite then concentrated by rotary evaporation at 43° C. The obtained residue was dissolved in dichloromethane/heptane 1:1 (400 mL) and the formed orange solid was removed by filtration. The greenish mother liquor was evaporated to give 115.5 g of a green foam. Dissolved this material in 500 mL of 1:1 dichloromethane/hexanes then loaded onto a 3 kg silica gel column (column volume=4800 mL, flow rate=900 mL/min). Ran a gradient of 2% to 9% EtOAc in hexanes over 43 minutes (8 column volumes), then ran at 9% EtOAc until the product finished eluting giving 77.8 g of impure product. This material was co-evaporated with methanol (˜500 mL) then diluted with methanol (200 mL) to give 234.5 g of a methanolic solution, which was halved and each half was purified by reverse phase chromatography (3.8 kg C₁₈ column, column volume=3300 mL, flow rate=375 mL/min, loaded as solution in methanol). Ran the column at 55% acetonitrile for ˜5 minutes (0.5 column volumes), then at a gradient of 55% to 100% acetonitrile in water over ˜170 minutes (19-20 column volumes), then held at 100% acetonitrile until the product and impurities finished eluting. Clean product fractions from both columns were combined and concentrated by rotary evaporation then transferred with ethanol into 5 L flask, evaporated and carefully dried (becomes a foam) to give as a mixture of olefin isomers, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (55.5 g, 53%). ESI-MS m/z calc. 728.26447, found 729.0 (M+1)⁺; Retention time: 3.82 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 3: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (11.7 g, 16.06 mmol) was dissolved in stirring ethanol (230 mL) and cycled the flask 3 times vacuum/nitrogen and treated with 10% Pd/C (50% water wet, 2.2 g of 5% w/w, 1.034 mmol). The mixture was cycled 3 times between vacuum/nitrogen and 3 times between vacuum/hydrogen. The mixture was then stirred strongly under hydrogen (balloon) for 7.5 h. The catalyst was removed by filtration, replaced with fresh 10% Pd/C (50% water wet, 2.2 g of 5% w/w, 1.034 mmol) and stirred vigorously under hydrogen (balloon) overnight. Then, the catalyst was removed again by filtration, the filtrate evaporated and the residue (11.3 g, 1 g set aside) was dissolved in ethanol (230 mL) charged with fresh 10% Pd/C (50% water wet, 2.2 g of 5% w/w, 1.034 mmol) and stirred vigorously under hydrogen (balloon) for 6 h, recharged again with fresh 10% Pd/C (50% water wet, 2.2 g of 5% w/w, 1.034 mmol) and stirred vigorously under hydrogen (balloon) overnight. The catalyst was removed by filtration and the filtrate was evaporated (10 g of residue obtained). This crude material (10 g+1 g set aside above) was purified by silica gel chromatography (330 g column, liquid load in dichloromethane) with a linear gradient of 0% to 15% ethyl acetate in hexane until the product eluted followed by 15% to 100% ethyl acetate in hexane to giving, as a colorless foam, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (9.1 g, 78%). ESI-MS m/z calc. 730.2801, found 731.0 (M+1)⁺; Retention time: 3.89 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 4: (6R,12R)-17-amino-12-methyl-6,15-bis(trifluoromethyl)-13,19-dioxa-3,4,18-triazatricyclo[12.3.1.12,5] nonadeca-1(18),2,4,14,16-pentaen-6-ol

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (8.6 g, 11.77 mmol) was dissolved in ethanol (172 mL) then the flask was cycled 3 times between vacuum/nitrogen. Treated the mixture with 10% Pd/C (50% water wet, 1.8 g of 5% w/w, 0.8457 mmol) then cycled 3 times between vacuum/nitrogen and 3 times between vacuum/hydrogen and then stirred vigorously under hydrogen (balloon) at room temperature for 18 h. The mixture was cycled 3 times between vacuum/nitrogen, filtered over Celite washing with ethanol and then the filtrate was evaporated to give 7.3 g of tert-butyl N-tert-butoxycarbonyl-N-[(6R,12R)-6-hydroxy-12-methyl-6,15-bis(trifluoromethyl)-13,19-dioxa-3,4,18-triazatricyclo[12.3.1.12,5]nonadeca-1(18),2,4,14,16-pentaen-17-yl]carbamate an off-white solid. 1H NMR and MS confirmed the expected product. CFTR modulatory activity was confirmed using a standard Ussing Chamber Assay for CFTR potentiator activity.

### OTHER EMBODIMENTS

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

1. A compound of Formula I: 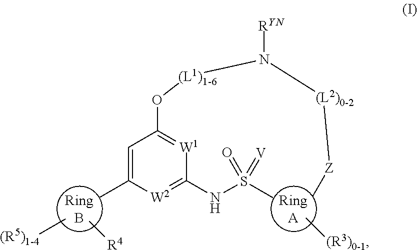
a tautomer thereof, a deuterated derivative of the compound or tautomer, or a pharmaceutically acceptable salt of any of the foregoing, wherein:
Ring A is selected from:
C₆-C₁₀ aryl,
C₃-C₁₀ cycloalkyl,
3- to 10-membered heterocyclyl, and
5- to 10-membered heteroaryl;
Ring B is selected from:
C₆-C₁₀ aryl,
C₃-C₁₀ cycloalkyl,
3- to 10-membered heterocyclyl, and
5- to 10-membered heteroaryl;
V is selected from O and NH;
W¹ is selected from N and CH;
W² is selected from N and CH, provided that at least one of W¹ and W² is N;
Z is selected from O, NR^{ZN}, and C(R^{ZC})₂, provided that when L² is absent, Z is C(R^{ZC})₂;
each L¹ is independently selected from C(R^{L1})₂ and 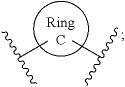
each L² is independently selected from C(R^{L2})₂;
Ring C is selected from C₆-C₁₀ aryl optionally substituted with 1-3 groups independently selected from:
halogen,
C₁-C₆ alkyl, and
N(R^{N})₂;
each R³ is independently selected from:
halogen,
C₁-C₆ alkyl,
C₁-C₆ alkoxy,
C₃-C₁₀ cycloalkyl,
C₆-C₁₀ aryl optionally substituted with 1-3 groups independently selected from C₁-C₆ alkyl, and
3- to 10-membered heterocyclyl;
R⁴ is selected from hydrogen and C₁-C₆ alkyl;
each R⁵ is independently selected from:
hydrogen,
halogen,
hydroxyl,
N(R^{N})₂,
—SO-Me,
CH═C(R^{LC})₂, wherein both R^{LC} are taken together to form a C₃-C₁₀ cycloalkyl,
C₁-C₆ alkyl optionally substituted with 1-3 groups independently selected from:
hydroxyl,
C₁-C₆ alkoxy optionally substituted with 1-3 groups independently selected from C₁-C₆ alkoxy and C₆-C₁₀ aryl,
C₃-C₁₀ cycloalkyl,
—(O)₀₋₁—(C₆-C₁₀ aryl) optionally substituted with 1-3 groups independently selected from C₁-C₆ alkyl and C₁-C₆ alkoxy,
3- to 10-membered heterocyclyl, and
N(R^{N})₂,
C₁-C₆ alkoxy optionally substituted with 1-3 groups independently selected from:
halogen,
C₆-C₁₀ aryl, and
C₃-C₁₀ cycloalkyl optionally substituted with 1-3 groups independently selected from C₁-C₆ fluoroalkyl,
C₁-C₆ fluoroalkyl,
C₃-C₁₀ cycloalkyl,
C₆-C₁₀ aryl, and
3- to 10-membered heterocyclyl;
R^{YN} is selected from:
C₃-C₁₀ cycloalkyl optionally substituted with 1-3 groups independently selected from:
hydroxyl,
oxo,
halogen,
cyano,
N(R^{N})₂,
C₁-C₆ alkyl optionally substituted with 1-3 groups independently selected from:
hydroxyl,
oxo,
N(R^{N})₂,
C₁-C₆ alkoxy, and
C₆-C₁₀ aryl,
C₁-C₆ alkoxy optionally substituted with 1-3 groups independently selected from halogen, oxo, C₆-C₁₀ aryl, and N(R^{N})₂,
halogen,
C₃-C₁₀ cycloalkyl,
3- to 10-memember heterocyclyl optionally substituted with 1-3 groups independently selected from C₁-C₆ alkyl,
5- to 10-membered heteroaryl optionally substituted with 1-3 groups independently selected from:
hydroxyl,
cyano,
oxo,
halogen,
N(R^{N})₂,
C₁-C₆ alkyl optionally substituted with 1-3 groups independently selected from hydroxyl, oxo, C₁-C₆ alkoxy, and N(R^{N})₂,
C₁-C₆ alkoxy optionally substituted with 1-3 groups independently selected from hydroxyl, C₁-C₆ alkoxy, N(R^{N})₂, and C₃-C₁₀ cycloalkyl,
C₁-C₆ fluoroalkyl,
(O)₀₋₁—(C₃-C₁₀ cycloalkyl) optionally substituted with 1-3 groups independently selected from C₁-C₆ alkyl,
C₆-C₁₀ aryl, and
3- to 10-membered heterocyclyl optionally substituted with 1-3 groups independently selected from C₁-C₆ alkyl,
C₆-C₁₀ aryl,
3- to 10-membered heterocyclyl optionally substituted with 1-3 groups independently selected from:
oxo,
C₁-C₆ alkyl optionally substituted with 1-3 groups independently selected from:
oxo,
hydroxyl,
N(R^{N})₂,
C₁-C₆ alkoxy optionally substituted with 1-3 groups independently selected from halogen and C₆-C₁₀ aryl, and
(O)₀₋₁—(C₃-C₁₀ cycloalkyl),
C₁-C₆ fluoroalkyl,
C₃-C₁₀ cycloalkyl optionally substituted with 1-3 groups independently selected from halogen, and
3- to 10-membered heterocyclyl, and
5- to 10-membered heteroaryl optionally substituted with 1-3 groups independently selected from:
halogen,
C₁-C₆ alkyl optionally substituted with 1-3 groups independently selected from oxo, C₁-C₆ alkoxy, and N(R^{N})₂, and
3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl);
R^{ZN} is selected from:
hydrogen,
C₁-C₉ alkyl optionally substituted with 1-3 groups independently selected from:
hydroxyl,
oxo,
cyano,
C₁-C₆ alkoxy optionally substituted with 1-3 groups independently selected from halogen and C₁-C₆ alkoxy,
N(R^{N})₂,
SO₂Me,
C₃-C₁₀ cycloalkyl optionally substituted with 1-3 groups independently selected from:
hydroxyl,
C₁-C₆ alkyl optionally substituted with 1-3 groups independently selected from hydroxyl, oxo, C₁-C₆ alkoxy, C₆-C₁₀ aryl, and N(R^{N})₂,
C₁-C₆ fluoroalkyl,
C₁-C₆ alkoxy,
COOH,
N(R^{N})₂,
C₆-C₁₀ aryl, and
3- to 10-membered heterocyclyl optionally substituted with 1-3 groups independently selected from oxo and C₁-C₆ alkyl,
C₆-C₁₀ aryl optionally substituted with 1-3 groups independently selected from:
halogen,
hydroxyl,
cyano,
SiMe₃,
SO₂Me,
SF₅,
N(R^{N})₂,
P(O)Me₂,
(O)₀₋₁—(C₃-C₁₀ cycloalkyl) optionally substituted with 1-3 groups independently selected from C₁-C₆ fluoroalkyl,
C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
C₁-C₆ alkoxy optionally substituted with 1-3 groups independently selected from hydroxyl, oxo, N(R^{N})₂, and C₆-C₁₀ aryl,
C₁-C₆ fluoroalkyl,
3- to 10-membered heterocyclyl optionally substituted with 1-3 groups independently selected from C₁-C₆ alkyl,
—(O)₀₋₁—(C₆-C₁₀ aryl), and
—(O)₀₋₁—(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
3- to 10-membered heterocyclyl optionally substituted with 1-4 groups independently selected from:
hydroxyl,
oxo,
N(R^{N})₂,
C₁-C₆ alkyl optionally substituted with 1-3 groups independently selected from oxo and C₁-C₆ alkoxy,
C₁-C₆ alkoxy,
C₁-C₆ fluoroalkyl,
C₆-C₁₀ aryl optionally substituted with 1-3 groups independently selected from halogen, and
5- to 10-membered heteroaryl, and
5- to 10-membered heteroaryl optionally substituted with 1-3 groups independently selected from:
hydroxyl,
cyano,
oxo,
halogen,
B(OH)₂,
N(R^{N})₂,
C₁-C₆ alkyl optionally substituted with 1-3 groups independently selected from hydroxyl, oxo, C₁-C₆ alkoxy (optionally substituted with 1-3 —SiMe₃), and N(R^{N})₂,
C₁-C₆ alkoxy optionally substituted with 1-3 groups independently selected from hydroxyl, oxo, C₁-C₆ alkoxy, N(R^{N})₂, and C₃-C₁₀ cycloalkyl,
C₁-C₆ fluoroalkyl,
—(O)₀₋₁—(C₃-C₁₀ cycloalkyl) optionally substituted with 1-3 groups independently selected from C₁-C₆ alkyl,
—(O)₀₋₁—(C₆-C₁₀ aryl),
—(O)₀₋₁—(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, and 3- to 10-membered heterocyclyl (optionally substituted with 1-3 groups independently selected from C₁-C₆ fluoroalkyl), and
5- to 10-membered heteroaryl optionally substituted with 1-4 groups independently selected from C₁-C₆ alkyl and C₃-C₁₀ cycloalkyl,
C₁-C₆ fluoroalkyl,
C₃-C₁₀ cycloalkyl optionally substituted with 1-3 groups independently selected from:
hydroxyl,
oxo,
halogen,
cyano,
N(R^{N})₂,
C₁-C₆ alkyl optionally substituted with 1-3 groups independently selected from:
hydroxyl,
oxo,
N(R^{N})₂,
C₁-C₆ alkoxy, and
C₆-C₁₀ aryl,
C₁-C₆ alkoxy optionally substituted with 1-3 groups independently selected from halogen, oxo, C₆-C₁₀ aryl, and N(R^{N})₂,
halogen,
C₃-C₁₀ cycloalkyl,
3- to 10-memember heterocyclyl optionally substituted with 1-3 groups independently selected from C₁-C₆ alkyl,
5- to 10-membered heteroaryl optionally substituted with 1-3 groups independently selected from:
hydroxyl,
cyano,
oxo,
halogen,
N(R^{N})₂,
C₁-C₆ alkyl optionally substituted with 1-3 groups independently selected from hydroxyl, oxo, C₁-C₆ alkoxy, and N(R^{N})₂,
C₁-C₆ alkoxy optionally substituted with 1-3 groups independently selected from hydroxyl, C₁-C₆ alkoxy, N(R^{N})₂, and C₃-C₁₀ cycloalkyl,
C₁-C₆ fluoroalkyl,
—(O)₀₋₁—(C₃-C₁₀ cycloalkyl) optionally substituted with 1-3 groups independently selected from C₁-C₆ alkyl,
C₆-C₁₀ aryl, and
3- to 10-membered heterocyclyl optionally substituted with 1-3 groups independently selected from C₁-C₆ alkyl,
C₆-C₁₀ aryl,
3- to 10-membered heterocyclyl optionally substituted with 1-3 groups independently selected from:
oxo,
C₁-C₆ alkyl optionally substituted with 1-3 groups independently selected from:
oxo,
hydroxyl,
N(R^{N})₂,
C₁-C₆ alkoxy optionally substituted with 1-3 groups independently selected from halogen and C₆-C₁₀ aryl, and
—(O)₀₋₁—(C₃-C₁₀ cycloalkyl),
C₁-C₆ fluoroalkyl,
C₃-C₁₀ cycloalkyl optionally substituted with 1-3 groups independently selected from halogen, and
3- to 10-membered heterocyclyl,
5- to 10-membered heteroaryl optionally substituted with 1-3 groups independently selected from:
halogen,
C₁-C₆ alkyl optionally substituted with 1-3 groups independently selected from oxo, C₁-C₆ alkoxy, and N(R^{N})₂, and
3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
R^{F};
each R^{ZC} is independently selected from:
hydrogen,
C₁-C₆ alkyl optionally substituted with 1-3 groups independently selected from C₆-C₁₀ aryl (optionally substituted with 1-3 groups independently selected from C₁-C₆ alkyl),
C₆-C₁₀ aryl optionally substituted with 1-3 groups independently selected from C₁-C₆ alkyl, and
R^{F};
or two R^{ZC} are taken together to form an oxo group;
each R^{L1} is independently selected from:
hydrogen,
N(R^{N})₂, provided that two N(R^{N})₂ are not bonded to the same carbon,
C₁-C₉ alkyl optionally substituted with 1-3 groups independently selected from:
halogen,
hydroxyl,
oxo,
N(R^{N})₂,
C₁-C₆ alkoxy optionally substituted with 1-3 groups independently selected from C₆-C₁₀ aryl,
C₃-C₁₀ cycloalkyl optionally substituted with 1-3 groups independently selected from halogen and C₁-C₆ fluoroalkyl,
C₆-C₁₀ aryl optionally substituted with 1-3 groups independently selected from C₁-C₆ alkyl, and
3- to 10-membered heterocyclyl optionally substituted with 1-3 groups independently selected from C₁-C₆ alkyl (optionally substituted with 1-3 groups independently selected from hydroxyl and oxo),
C₃-C₁₀ cycloalkyl,
C₆-C₁₀ aryl optionally substituted with 1-4 groups independently selected from:
halogen,
cyano,
SiMe₃,
POMe₂,
C₁-C₇ alkyl optionally substituted with 1-3 groups independently selected from:
hydroxyl,
oxo,
cyano,
SiMe₃,
N(R^{N})₂, and
C₃-C₁₀ cycloalkyl optionally substituted with 1-3 groups independently selected from C₁-C₆ fluoroalkyl,
C₁-C₆ alkoxy optionally substituted with 1-3 groups independently selected from:
C₃-C₁₀ cycloalkyl optionally substituted with 1-3 groups independently selected from C₁-C₆ fluoroalkyl, and
C₁-C₆ alkoxy,
C₁-C₆ fluoroalkyl,
C₃-C₁₀ cycloalkyl optionally substituted with 1-3 groups independently selected from C₁-C₆ alkyl and C₁-C₆ fluoroalkyl,
C₆-C₁₀ aryl,
3- to 10-membered heterocyclyl optionally substituted with 1-3 groups independently selected from C₁-C₆ alkyl, and
5- to 10-membered heteroaryl,
3- to 10-membered heterocyclyl optionally substituted with 1-3 groups independently selected from:
C₁-C₆ alkyl optionally substituted with 1-3 groups independently selected from:
oxo, and
C₁-C₆ alkoxy,
5- to 10-membered heteroaryl optionally substituted with 1-3 groups independently selected from:
C₁-C₆ alkyl optionally substituted with 1-3 groups independently selected from:
C₃-C₁₀ cycloalkyl optionally substituted with 1-3 groups independently selected from C₁-C₆ fluoroalkyl, and
C₆-C₁₀ aryl optionally substituted with 1-3 groups independently selected from C₁-C₆ alkyl, and
R^{F};
or two R^{L1} on the same carbon atom are taken together to form an oxo group;
each R^{L2} is independently selected from hydrogen and R^{F};
or two R^{L2} on the same carbon atom are taken together to form an oxo group;
each R^{N} is independently selected from:
hydrogen,
C₁-C₈ alkyl optionally substituted with 1-3 groups independently selected from:
oxo,
halogen,
hydroxyl,
NH₂,
NHMe,
NMe₂,
NHCOMe,
C₁-C₆ alkoxy optionally substituted with 1-3 groups independently selected from C₆-C₁₀ aryl,
—(O)₀₋₁—(C₃-C₁₀ cycloalkyl),
C₆-C₁₀ aryl optionally substituted with 1-3 groups independently selected from halogen and C₁-C₆ alkyl,
3- to 14-membered heterocyclyl optionally substituted with 1-4 groups independently selected from oxo and C₁-C₆ alkyl, and
5- to 14-membered heteroaryl optionally substituted with 1-4 groups independently selected from oxo and C₁-C₆ alkyl,
C₃-C₁₀ cycloalkyl optionally substituted with 1-3 groups independently selected from:
hydroxyl,
NH₂,
NHMe, and
C₁-C₆ alkyl optionally substituted with 1-3 groups independently selected from hydroxyl,
C₆-C₁₀ aryl, and
3- to 10-membered heterocyclyl;
or two R^{N} on the same nitrogen atom are taken together with the nitrogen to which they are bonded to form a 3- to 10-membered heterocyclyl optionally substituted with 1-3 groups selected from:
hydroxyl,
oxo,
cyano,
C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
C₁-C₆ alkoxy, and
C₁-C₆ fluoroalkyl;
or one R⁴ and one R^{L1} are taken together to form a C₆-C₈ alkylene;
when R^{F} is present, two R^{F} taken together with the atoms to which they are bonded form a group selected from:
C₃-C₁₀ cycloalkyl optionally substituted with 1-3 groups independently selected from C₁-C₆ alkyl,
C₆-C₁₀ aryl optionally substituted with 1-3 groups independently selected from:
halogen,
C₁-C₆ alkyl,
N(R^{N})₂, and
3- to 10-membered heterocyclyl optionally substituted with 1-3 groups independently selected from hydroxyl,
3- to 11-membered heterocyclyl optionally substituted with 1-3 groups independently selected from:
oxo,
N(R^{N})₂,
C₁-C₉ alkyl optionally substituted with 1-4 groups independently selected from:
oxo,
halogen,
hydroxyl,
N(R^{N})₂,
—SO₂—(C₁-C₆ alkyl),
C₁-C₆ alkoxy optionally substituted with 1-3 groups independently selected from halogen and C₆-C₁₀ aryl,
C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
—(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N},
—O-(5- to 12-membered heteroaryl) optionally substituted with 1-3 groups independently selected from C₆-C₁₀ aryl (optionally substituted with 1-3 groups independently selected from halogen) and C₁-C₆ alkyl, and
5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
C₃-C₁₂ cycloalkyl optionally substituted with 1-4 groups independently selected from halogen, C₁-C₆ alkyl, and C₁-C₆ fluoroalkyl,
C₆-C₁₀ aryl,
3- to 10-membered heterocyclyl, and
5- to 10-membered heteroaryl optionally substituted with 1-3 groups independently selected from C₁-C₆ alkoxy and C₁-C₆ fluoroalkyl, and
5- to 12-membered heteroaryl optionally substituted with 1-3 groups independently selected from C₁-C₆ alkyl and C₁-C₆ fluoroalkyl;
with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.

2. A compound of Formula Ia: 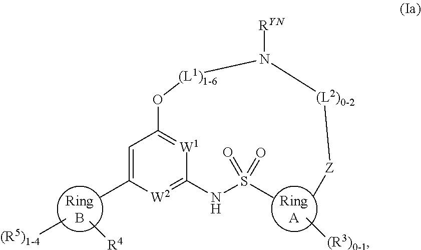
a tautomer thereof, a deuterated derivative of the compound or tautomer, or a pharmaceutically acceptable salt of any of the foregoing, wherein Ring A, Ring B, W¹, W², Z, L¹, L², R³, R⁴, R⁵, and R^{YN} are defined as according to claim 1, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.

3. A compound of Formula IIa: 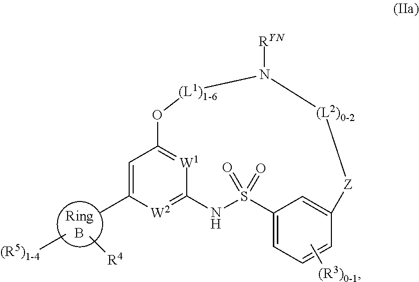
a tautomer thereof, a deuterated derivative of the compound or tautomer, or a pharmaceutically acceptable salt of any of the foregoing, wherein Ring B, W¹, W², Z, L¹, L², R³, R⁴, R⁵, and R^{YN} are defined as according to claim 1, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.

4. A compound of Formula IIb: 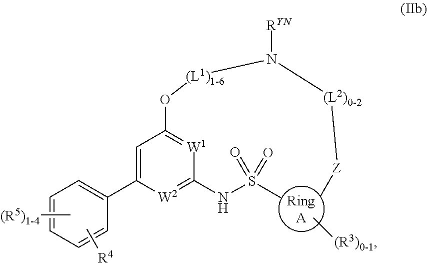
a tautomer thereof, a deuterated derivative of the compound or tautomer, or a pharmaceutically acceptable salt of any of the foregoing, wherein Ring A, W¹, W², Z, L¹, L², R³, R⁴, R⁵, and R^{YN} are defined as according to claim 1, with the proviso that the compound is not selected from:
(11R)-6-(2,6-Dimethylphenyl)-11-(2-methylpropyl)-12-{spiro[2.3]hexan-5-yl}-9-oxa-2 λ6-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2 λ6-thia-3,5,12,19-tetraazatricyclo[12.3.1.14,8]nonadeca-1(17),4(19),5,7,14(18),15-hexaene-2,2,13-tri one,
(11R)-6-(2,6-Dimethylphenyl)-11-isobutyl-2,2-dioxo-12-(4,4,5,6,6-pentadeuteriospiro[2.3]hexan-5-yl)-9-oxa-2 λ6-thia-3,5,12,19-tetrazatricyclo[12.3.1.14,8]nonadeca-1(18),4, 6, 8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2a,6-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2 λ6-thia-3,5,12,19-tetraazatricyclo[12.3.1.14,8]nonadeca-1(17),4(19), 5,7,14(18),15-hexaene-2,2,13-tri one.

5. A compound of Formula III: 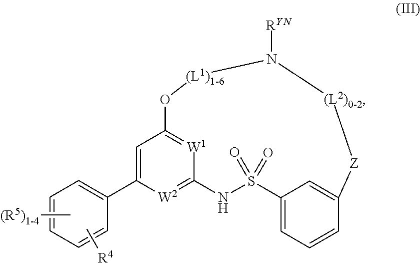
a tautomer thereof, a deuterated derivative of the compound or tautomer, or a pharmaceutically acceptable salt of any of the foregoing, wherein W¹, W², Z, L¹, L², R⁴, R⁵, and R^{YN} are defined as according to claim 1, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.

6. A compound of Formula IV: 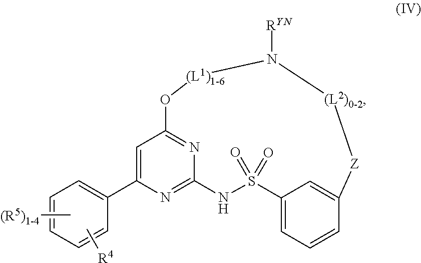
a tautomer thereof, a deuterated derivative of the compound or tautomer, or a pharmaceutically acceptable salt of any of the foregoing, wherein Z, L¹, L², R⁴, R⁵, and R^{YN} are defined as according to claim 1, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.

7. A compound of Formula V: 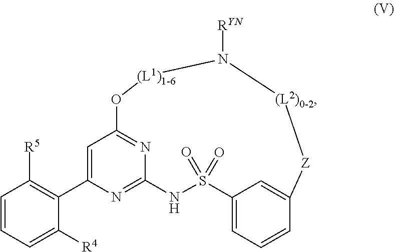
a tautomer thereof, a deuterated derivative of the compound or tautomer, or a pharmaceutically acceptable salt of any of the foregoing, wherein Z, L¹, L², R⁴, R⁵, and R^{YN} are defined as according to claim 1, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.

8. A compound of Formula VI: 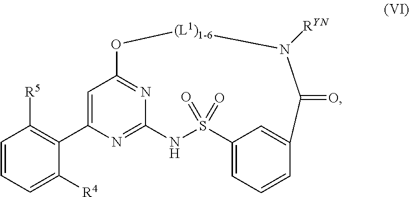
a tautomer thereof, a deuterated derivative of the compound or tautomer, or a pharmaceutically acceptable salt of any of the foregoing, wherein L¹, R⁴, R⁵, and R^{YN} are defined as according to claim 1, with the proviso that the compound is not selected from:
(11R)-6-(2,6-Dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-(2-methylpropyl)-12-[(1,1,2,2-tetradeutero)spiro[2.3]hexan-5-yl]-9-oxa-2λ⁶-thia-3,5,12,19-tetraazatricyclo[12.3.1.14,8]nonadeca-1(17),4(19),5,7,14(18),15-hexaene-2,2,13-trione,
(11R)-6-(2,6-Dimethylphenyl)-11-isobutyl-2,2-dioxo-12-(4,4,5,6,6-pentadeuteriospiro[2.3]hexan-5-yl)-9-oxa-2λ⁶-thia-3,5,12,19-tetrazatricyclo[12.3.1.14,8]nonadeca-1(18),4,6,8(19),14,16-hexaen-13-one,
(11R)-12-(5-Deuteriospiro[2.3]hexan-5-yl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4,6,8(19),14,16-hexaen-13-one, and
(11R)-6-[2,6-di(trideutero)methylphenyl]-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione.

9. The compound, tautomer, deuterated derivative, or pharmaceutically acceptable salt according to any one of claims 1 to 8, selected from compounds of Formulae I, Ia, IIa, IIb, III, IV, V, and VI, deuterated derivatives thereof, and pharmaceutically acceptable salts of any of the foregoing.

10. The compound, tautomer, deuterated derivative, or pharmaceutically acceptable salt according to any one of claims 1 to 8, selected from Compounds 1-474 (Tables 8,9,10,11), Compounds 475-506 (Table 7), Compounds 507 and 508 (Table 12), deuterated derivatives thereof, and pharmaceutically acceptable salts of any of the foregoing.

11. A pharmaceutical composition comprising the compound, tautomer, deuterated derivative, or pharmaceutically acceptable salt according to any one of claims 1 to 10, and a pharmaceutically acceptable carrier.

12. The pharmaceutical composition according to claim 11, further comprising one or more additional therapeutic agents.

13. A method of treating cystic fibrosis comprising administering to a patient in need thereof the compound, tautomer, deuterated derivative, or pharmaceutically acceptable salt according to any one of claims 1 to 10, or a pharmaceutical composition according to claim 11 or claim 12.

14. The method according to claim 13, further comprising administering to the patient one or more additional therapeutic agents prior to, concurrent with, or subsequent to the compound, tautomer, deuterated derivative, or pharmaceutically acceptable salt according to any one of claims 1 to 10, or the pharmaceutical composition according to claim 11 or claim 12.

15. The method according to claim 14, wherein the one or more additional therapeutic agents is a compound selected from tezacaftor, ivacaftor, deutivacaftor, lumacaftor, and pharmaceutically acceptable salts thereof.

16. The compound, tautomer, deuterated derivative, or pharmaceutically acceptable salt according to any one of claims 1 to 10, or the pharmaceutical composition according to claim 11 or claim 12, for use in the treatment of cystic fibrosis.

17. The compound, tautomer, deuterated derivative, or pharmaceutically acceptable salt according to any one of claims 1 to 10, or the pharmaceutical composition according to claim 11 or claim 12, for use in the manufacture of a medicament for the treatment of cystic fibrosis.
